(19) United States
(12) Patent Application Publication
(10) Pub. No.: US 2005/0197492 A1
(43) Pub. Date: Sep. 8, 2005

(54) Crystal structure of VEGFRKD: ligand complexes and methods of use thereof

(75) Inventors: Steven Lee Bender, Oceanside, CA (US); Robert Steven Kania, San Diego, CA (US); Michele Ann McTigue, Encinitas, CA (US); Cynthia Louise Palmer, La Mesa, CA (US); Chris Pinko, San Diego, CA (US); John Wickersham, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/824,982

(22) Filed: Apr. 15, 2004

Related U.S. Application Data
(60) Provisional application No. 60/463,957, filed on Apr. 17, 2003.

(51) Int. Cl. C07K 14/71

(52) U.S. Cl. 530/350

(57) ABSTRACT

Polypeptides containing the kinase domain of a VEGFR are described. Also described are crystal structures of these polypeptides, including the crystal structures of VEGFR2KD:ligand complexes. The atomic coordinates derived from the crystal structures provide a three-dimensional description of the ligand-binding pocket of the kinase domain useful in drug discovery and design for the identification and design of modulators of kinase activity.

This application claims the benefit of U.S. Provisional Application Ser. No. 60/463,957, filed Apr. 17, 2003, the disclosure of which is incorporated herein by reference in its entirety.

### BACKGROUND OF THE INVENTION

Protein kinases are a family of enzymes that catalyze phosphorylation of the hydroxyl group of specific tyrosine, serine, or threonine residues in proteins. Typically, such phosphorylation dramatically perturbs the function of the protein, and thus protein kinases are pivotal in the regulation of a wide variety of cellular processes, including metabolisim, cell proliferation, cell differentiation, and cell survival. Of the many different cellular functions in which the activity of protein kinases is known to be required, some processes represent attractive targets for therapeutic intervention for certain disease states. One example is angiogenesis, the sprouting of new blood vessels from existing vasculature.

Vascular endothelial growth factors (VEGFs) and their receptor tyrosine kinases (VEGFRs) are key components in angiogenesis. Human VEGFRs are known to include, for example, the homologous proteins VEGFR-1 (also referred to as “Flt-1”), VEGFR-2 (also referred to as “KDR”), and VEGFR-3 (also referred to as “Flt-4”). VEGFR-1 and VEGFR-2 are expressed preferentially on vascular endothelial cells and VEGFR-2 has been associated with the proliferation and survival of endothelial cells. The extracellular domain of VEGFR-2 binds the potent angiogenic growth factor VEGF and mediates the subsequent signal transduction through activation of its intracellular kinase activity. VEGFR-3 is expressed preferentially on lymphatic endothelial cells and is involved in lymphangiogenesis. (see PCT international application publication number WO 95/21613; Gerwins et al. (2000) Crit. Rev. Oncology/Hematology 34: 185-194; Ferrara and Davis-Smyth (1997) Endocrine Rev. 18(1): 4-25.

Angiogenesis is essential for embryonic development and other normal physiological processes such as wound healing and formation of the corpus luteum, endometrium and placenta. However, when angiogenesis occurs at an inappropriate time or location, numerous disease states and other undesirable conditions sometimes arise. For example, angiogenesis is involved in other diseases and conditions, including arthritis and atherosclerotic plaques, diabetic retinopathy, neovascular glaucoma, trachoma and corneal graft neovascularization, psoriasis, scleroderma, hemangioma and hypertrophic scarring, vascular adhesions and angiofibroma. Angiogenesis is also essential for solid tumor growth and metastasis. Folkman (1990) J. Nat'l. Cancer Inst. 82: 4-6; Kim et al. (1993) Nature 362: 841-844; Hori et al. (1991) Cancer Res. 51: 6180-6184; Millauer et al. (1994) Nature 367: 576-579; Sim et al. (1997) Cancer Res. 57: 1329-1334. Tumor cells are believed to cause a local disruption of the delicate balance that normally exists between angiogenesis inhibitors and stimulators. According to this model, by producing angiogenesis stimulators, such as VEGF, tumors cause a local increase in the ratio of stimulators to inhibitors, which induce the formation of new blood vessels that carry oxygen and nutrients to the growing tumor. See, e.g., O'Reilly (1997) Regulation of Angiogenesis, Goldberg & Rosen, Eds., Birkhauser Verlag, Basel, pp. 273-294.

Thus, agents which are capable of modulating the kinase activity of VEGFRs, are desired and may be used to treat disorders related to vasculogenesis or angiogenesis. Such disorders include, but are not limited to, diabetes, diabetic retinopathy, hemangioma, glioma, melanoma, Kaposi's sarcoma and ovarian, breast, lung, pancreatic, prostate, colon and epidermoid cancer. One means of modulating a kinase activity of VEGFR is to identify enhancers or inhibitors to the VEGFR. Such identification has heretofore relied on serendipity and/or systematic screening of large numbers of natural and synthetic compounds. A superior method of drug development relies on structure assisted drug design. In this case, the three-dimensional structure of a peptide-inhibitor complex is determined and potential enhancers and/or potential inhibitors are screened and/or designed with the aid of computer modeling [Bugg et al., Scientific American, December: 92-98 (1993); West et al., TIPS, 16: 67-74 (1995); Dunbrack et al., Folding & Design, 2: 27-42 (1997)].

Like other protein kinases, the kinase domain (KD) of VEGFRs is known to contain a large flexible loop, referred to as the kinase activation loop, whose conformation has been postulated to regulate kinase activity. In many kinases, the conformation of the activation loop is controlled by the phosphorylation of specific activation loop residues. The loop can generally be defined as beginning with the conserved residues DFG and ending at the conserved APE sequence. In VEGFR2, this segment has been reported as corresponding to D1046-E1075 and as containing two tyrosines (Y1054 and Y1059). See U.S. Pat. No. 6,316,603. For protein kinases in general, in the activated (usually phosphorylated) form, this loop adopts an “open” conformation that permits the catalytically competent binding of Mg-ATP and substrates. In the non-activated form, generally non-phosphorylated, many different conformations of this loop have been reported for various kinases (reviewed in Johnson, L. N. et al., Cell 85: 149-158 (1996)).

The three-dimensional structure of unliganded, phophorylated VEGFR2 kinase domain (KD) has previously been reported. See U.S. Pat. No. 6,316,306; McTigue et al., Structure, Vol. 7, No. 3, pages 319-330 (1999). However, in this structure of the unliganded, phosphorylated VEGFR2KD a conformation for most of the kinase activation loop (residues 1048-1063) could not be modeled due to a lack of interpretable electron density, most likely caused by dynamic disorder.

The structures of serine/threonine and tyrosine protein kinases have been reported with and without bound ligands. See, e.g., the reported kinase structures found in Pargellis et al., Nat. Struct. Biol. 9(4): 268-272 (2002); Schindler et al., Science 289: 1938-1942 (2000); Knighton, D. R. et al., Science 253: 407-413 (1991); Johnson, L. N. et al., Cell 85: 149-158 (1996); Hubbard, S. R. et al., Nature 372: 746-754 (1994). However, for purposes of drug design, the reported structures differ significantly from that discovered for the VEGFR2KD:ligand complexes described herein.

Heretofore the three-dimensional structure of the unphosphorylated, liganded VEGFR2KD has remained unknown, essentially because no crystals have been produced of sufficient quality to allow the required X-ray crystallographic data to be obtained. Therefore, there is presently a need for obtaining an unphosphorylated VEGFRKD:ligand crystal complex of sufficient quality to allow such crystallographic data to be obtained. Further, there is a need for such crystals. There is also a need for the determination of the three-dimensional structure of such crystals. Finally, there is a need for procedures for related structural based drug design and/or screening based on the crystallographic data.

The citation of any reference herein should not be construed as an admission that such reference is available as “Prior Art” to the instant application.

### BRIEF DESCRIPTION OF THE DRAWINGS

FIG. 1. FIG. 1 is a ribbon representation of VEGFR2KD in complex with Compound 2. The ribbon is generated from the protein backbone coordinates (C, N, Cα, O) listed in Table 2. The approximate backbone positions of some residues are denoted by their residue numbers.

FIG. 2A. FIG. 2A is a stick representation of the ligand binding site of the VEGFR2KD:Compound 2 complex crystalline structure. Sidechain atoms are shown for only those residues that have been defined as forming part of the ligand binding site. This figure was generated using atomic coordinates listed in Table 2.

FIG. 2B. FIG. 2B is a stick representation of the ligand binding site of the VEGFR2KD:Compound 1 complex crystalline structure. Sidechain atoms are shown for only those residues that have been defined as forming part of the ligand binding site. This figure was generated using atomic coordinates listed in Table 1.

### SEQUENCE LISTING

SEQ ID NO: 1 Human VEGFR2 Nucleic Acid Sequence
   (Genbank Accession number 2655412)
SEQ ID NO: 2 Human VEGFR2 Amino Acid Sequence
   (Swiss-Prot Accession Number P35968)
SEQ ID NO: 3 Human VEGFR2KD Construct Amino Acid Sequence
SEQ ID NO: 4 Human VEGFR1 Amino Acid Sequence
   (Swiss-Prot Accession number P17948)
SEQ ID NO: 5 Human VEGFR3 Amino Acid Sequence
   (Swiss-Prot Accession number P35916)
SEQ ID NO: 6 PCR oligonucleotide primer sequence for Vcyt5
SEQ ID NO: 7 PCR oligonucleotide primer sequence for Vcyt3
SEQ ID NO: 8 PCR oligonucleotide primer sequence for Vcat5
SEQ ID NO: 9 PCR oligonucleotide primer sequence for Vcat3
SEQ ID NO: 10 Oligonucleotide primer sequence for construct Vcat
   (Δ G1172-G 1191)
SEQ ID NO: 11 Oligonucleotide for deletion of amino acid residues
   T940-E989 of SEQ ID NO: 1

### SUMMARY OF THE INVENTION

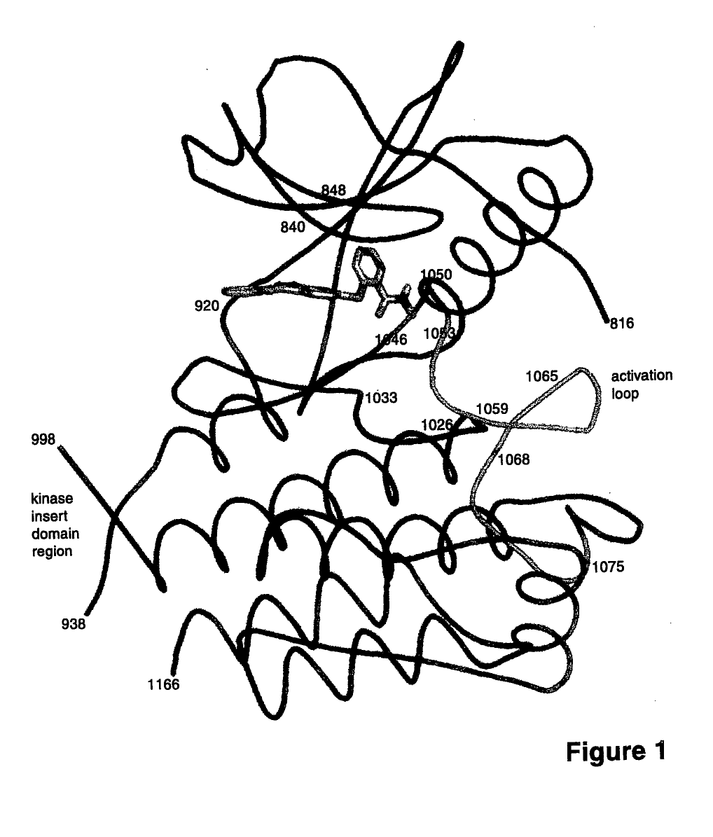

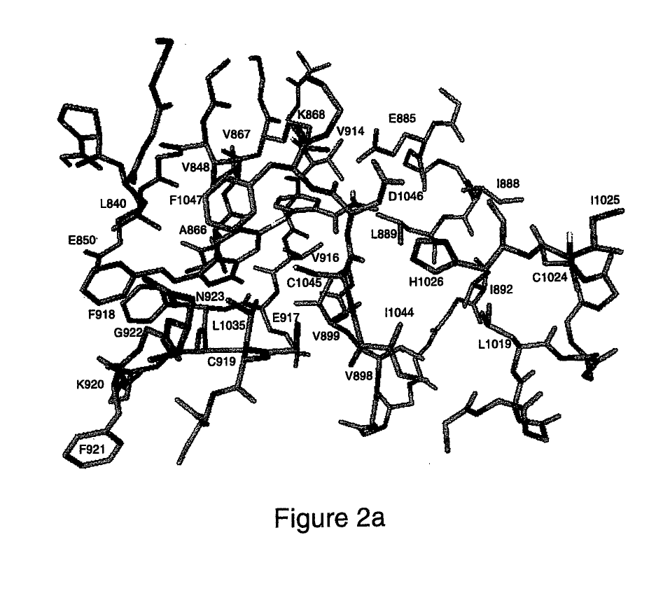

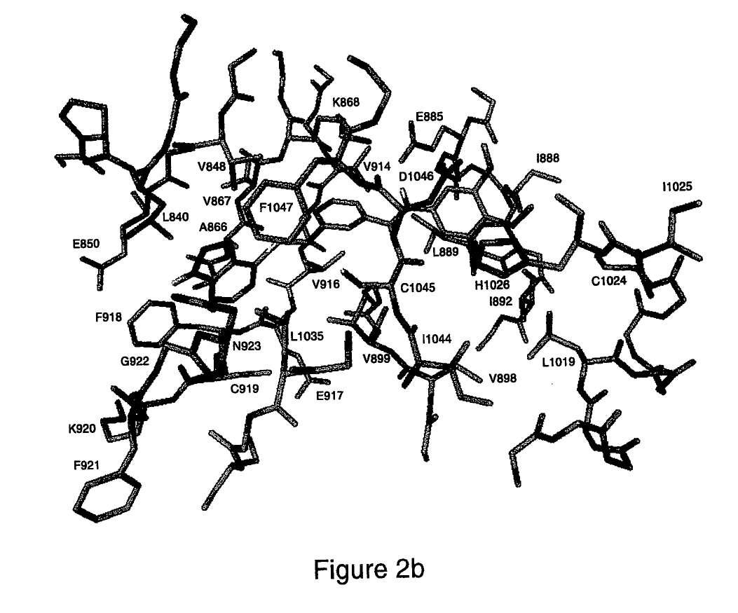

The invention relates to three-dimensional crystalline structures of VEGFRKD, or a structurally related peptide, and VEGFRKD:ligand complexes, particularly VEGFR2KD:ligand complexes. The invention also relates to crystallographic data derived therefrom, as set forth in Tables 1, 2, 3, 4, and/or 5, and use of the data in drug design and development. It has been discovered that the VEGFRKD, or structurally related peptide, comprise a ligand binding pocket that is defined by the atoms found in the structural coordinates set forth in Tables 1, 2, 3, 4 or 5, or in a related set of structural coordinates having a root mean square deviation of not more than about 0.90 Å away from the binding pocket Cα atoms of the ligand binding pocket defined by the atoms found in the structural coordinates set forth in Tables 1, 2, 3, 4 or 5. The ligand binding pocket is of approximate dimensions 12 Å×9 Å×25 Å, and is depicted in FIG. 1, FIG. 2A, or FIG. 2B. The ligand binding pocket is also defined by the structural coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2 or a conservatively substituted variant thereof. The VEGFRKS also comprises an activation loop defined by amino acid residues 1046 to 1075 of SEQ ID NO: 2, or a conservatively substituted variant thereof, as depicted in FIG. 1.

The invention also relates to a method of using the VEGFRKD:ligand crystalline structures of the invention, and atomic coordinates thereof, to identify a potential VEGFR modulator, or a modulator of a peptide structurally related to VEGFR, comprising:
(a) selecting a potential modulator by performing rational drug design using a three-dimensional structure defined by at least a portion of the atomic coordinates set forth in Tables 1, 2, 3, 4 or 5, or a related set of atomic coordinates having a root mean square deviation of not more than about 1.25 Å away from the core Cα atoms of the atomic coordinates set forth in Tables 1, 2, 3, 4 or 5;
(b) contacting the potential modulator with a vascular endothelial growth factor receptor (VEGFR) polypeptide; and
(c) detecting whether the potential modulator binds with the polypeptide.

The selection may be performed in conjunction with computer modeling.

The invention further includes a method for evaluating the potential of a chemical entity to associate with a VEGFRKD ligand binding pocket or VEGFRKD-like ligand binding pocket comprising:
(a) employing computational means to perform a fitting operation between the chemical entity and a ligand binding pocket defined by at least a portion of the atomic coordinates set forth in Tables 1, 2, 3, 4 or 5, or a related set of atomic coordinates having a root mean square deviation of not more than about 0.90 Å away from the core Cα atoms of the atomic coordinates as set forth in Tables 1, 2, 3, 4 or 5; and
(b) analyzing the results of the fitting operation to quantify the association between the chemical entity and the binding pocket.

Additionally, the invention is directed to a method for evaluating the ability of a chemical entity to associate with a molecule or molecular complex comprising a VEGFRKD or VEGFRKD-like ligand binding pocket, comprising
(a) constructing a computer model of a ligand binding pocket defined by at least a portion of the atomic coordinates set forth in Tables 1, 2, 3, 4 or 5, or a related set of atomic coordinates having a root mean square deviation of not more than about 0.90 Å away from the core Cα atoms of the atomic coordinates as set forth in Tables 1, 2, 3, 4 or 5;
(b) selecting a compound to be evaluated by a method selected from the group consisting of: (i) assembling molecular fragments into a compound, (ii) selecting a compound from a small molecule database, (iii) de novo ligand design of a compound, and (iv) modifying a known modulator, or a portion thereof, of a VEGFR;
(c) employing computational means to perform a fitting program operation between a computer model of the compound to be evaluated and the computer model of the ligand binding pocket to provide an energy-minimized configuration of the compound in the binding pocket; and
(d) evaluating the results of the fitting operation to quantify the association between the compound and the binding pocket model.

The invention also includes a method for identifying a modulator of a molecule comprising a VEGFRKD ligand binding pocket or VEGRKD-like ligand binding pocket, comprising:
(a) generating a three-dimensional structure of the VEGFRKD or VEGFRKD-like ligand binding pocket by applying at least a portion of the atomic coordinates set forth in Tables 1, 2, 3, 4 or 5, or a related set of atomic coordinates having a root mean square deviation of not more than about 0.90 Å away from the core Cα atoms of the atomic coordinates as set forth in Tables 1, 2, 3, 4 or 5, to a computer algorithm to generate a three-dimensional structural representation of the binding pocket;
(b) employing the three-dimensional structure to design or select the modulator;
(c) synthesizing or obtaining the modulator; and
(d) contacting the modulator with the molecule to determine the ability of the modulator to interact with the molecule.

Another method for identifying a modulator of a VEGFRKD ligand binding pocket or VEGFR2KD-like ligand binding pocket, comprises:
(a) constructing a computer model of the binding pocket;
(b) selecting a compound to be evaluated as a modulator by a method selected from the group consisting of: (i) assembling molecular fragments into a compound, (ii) selecting a compound from a small molecule database, (iii) de novo ligand design of a compound, and (iv) modifying a known inhibitor, or a portion thereof, of a VEGFR polypeptide;
(c) employing computational means to perform a fitting program operation between computer models of the compound to be evaluated and the binding pocket in order to provide an energy-minimized configuration of the compound in the binding pocket;
(d) evaluating the results of the fitting operation to quantity the association between the compound and the binding pocket model;
(e) synthesizing the compound; and

(f) contacting the compound with the molecule to determine the ability of the compound to modulate the kinase activity of the molecule,

wherein the ligand binding pocket is defined by at least a portion of the atomic coordinates set forth in Tables 1, 2, 3, 4 or 5, or a related set of atomic coordinates having a root mean square deviation of not more than about 0.90 Å away from the core Cα atoms of the atomic coordinates set forth in Tables 1, 2, 3, 4 or 5.

One ligand binding pocket that can be used in each of the foregoing methods of the invention is defined by the atomic coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2 or a conservatively substituted variant thereof.

### DETAILED DESCRIPTION OF THE INVENTION

Definitions

As used herein, the terms “comprising” and “including” are used in an open, non-limiting sense.

As used herein, the acronym “VEGFR” refers to a vascular endothelial growth factor receptor or proteins structurally related thereto, including, but not limited to VEGFR1 (SEQ ID NO: 4), VEGFR2 (SEQ ID NO: 2), and VEGFR3 (SEQ ID NO 5). The preferred receptor is VEGFR2. The VEGFR peptide may be modified as described herein.

As used herein, the acronym “VEGFRKD” refers to a VEGFR kinase domain, preferably the VEGFRKD of SEQ ID NO: 3, and conservatively substituted variants thereof. It is intended that VEGFRKD encompasses any peptide containing the domains of approximately amino acid residues 820 to 930 and 1002 to 1171 of SEQ ID NO: 2, and conservatively substituted variants thereof.

As used herein, Compound 1 refers to:

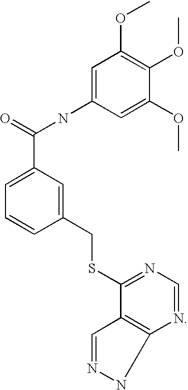

As used herein, Compound 2 refers to:

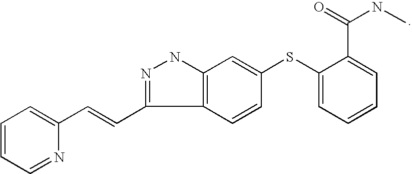

As used herein, Compound 3 refers to:

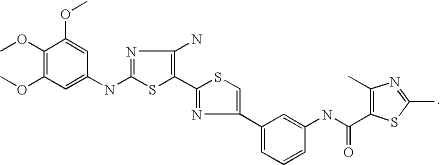

As used herein, Compound 4 refers to:

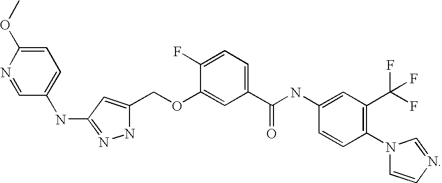

As used herein, Compound 5 refers to:

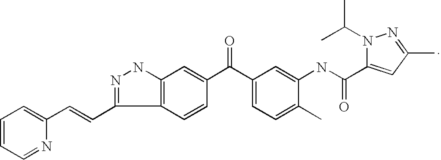

As used herein, the phrase “root mean square deviation” (“RMS”) means the square root of the arithmetic mean of the squares of the deviations from the mean and denotes a measure of the structural relationship between two or more species of proteins. It may be determined by, for example, superimposing one three-dimensional structure onto another, which may be solved by using, for example, X-ray crystallography or nuclear magnetic resonance (NMR), and then, calculating the difference in the RMS of the distance from the Cα and/or backbone (N, C, O, and Cα) trace (or atoms) of one protein to another protein in units of Angstroms (Å). The superimposition of three-dimensional structures may be performed using a molecular modeling program such as, for example, the Superimpose command in Insight II (Accelrys Inc., San Diego, Calif.), CNX (Accelrys Inc., San Diego, Calif.), XtalView™ (Scripps Research Institute, La Jolla, Calif.), SYBYL® (Tripos, Inc., St. Louis, Mo.), or O (Aarhus Univ., Denmark (Jones, T. A. et al., Acta Cryst. A47: 110-119 (1991)), or other related computer modeling programs or scripts, alone or in combination. For example, the Superimpose command in Insight II, performs a minimum RMS alignment of two molecules on selected sets of atoms from each molecule is then outputs the RMS deviation value between the selected atoms of the superimposed molecules. The closer the relationship between the three-dimensional structures, the smaller the RMS deviation value. Therefore, one embodiment of this invention is the three-dimensional structures of the VEGFR2KD complexes of the invention. An additional embodiment is crystals of a “structurally related” peptide and the three-dimensional structures thereof.

As used herein, “structurally related” protein or peptide refers to a protein or peptide that is defined by the atomic coordinates set forth in Table 1, Table 2, Table 3, Table 4, and/or Table 5, or by a related set of atomic coordinates having a root mean square deviation of from not more than about 1.25 Å from the core Cα atoms of the atomic coordinates set forth in Tables 1, 2, 3, 4, or 5. Preferably the root mean square deviation is not more than about 1.25 Å, more preferably not more than about 1.00 Å, and most preferably not more than about 0.75 Å. Examples of proteins structurally related to VEGFR2 include, but are not limited to, VEGFR1 and VEGFR3. Additionally, examples of proteins structurally related to VEGFR include, for example, platelet derived growth factor receptor (PDGFR), such as, PDGFRα, PDGFRβ, colony stimulating factor-1 receptor, and stem cell growth factor receptor.

Similarly, as used herein, “related set of structural coordinates” or “related set of atomic coordinates” refers to a set of structural (e.g. atomic) coordinates having a root mean square deviation of not more than about 1.25 Å from the Cα atoms of the structural coordinates a set forth in Table 1, Table 2, Table 3, Table 4, and/or Table 5. Preferably the root mean square deviation is not more than about 1.25 Å, more preferably not more than about 1.00 Å, and most preferably not more than about 0.75 Å.

As used herein, the phrase “chemical entity” refers to a chemical compound, a complex of at least two chemical compounds, or a fragment of such a compound or complex. Such entities are potential drug candidates and can be evaluated for their ability to modulate the activity of a VEGFR. The ability of an entity to bind to, or associate with, a VEGFRKD ligand-binding pocket, depends on the features of the entity. Assays to determine if a compound binds to the kinase domain are known in the art, such as those exemplified in U.S. Pat. No. 6,316,603.

As used herein, the term “ligand” means a molecule that binds to or associates with an enzyme and can be used to mean a VEGFR activity inhibitor or enhancer.

As used herein, the terms “modulator” and “modulatory,” and variations thereof, are used in an open, non-limiting sense. Preferably, the modulator is an inhibitor or enhancer of a VEGFR activity whereby the activity is either (1) decreased, stopped, prevented, slowed, or retarded, or (2) increased, encouraged, or sped up, respectively. The preferred modulator is an inhibitor. The most preferred modulator is an inhibitor of kinase activity. The kinase domain ligand binding pocket may be used to design molecules which bind at the site and modulate, for example, inhibit or enhance, kinase activity. The modulators, preferably, inhibitors, can then be developed into therapeutics for treating diseases, such as angiogenic or vascular diseases for which VEGFRs are causative factors.

As used herein, the term “inhibitor” or “inhibit” (or variations thereof) refers to a ligand such as a compound or substance that lowers, reduces, decreases, prevents, diminishes, stops or negatively interferes with a VEGFR activity. Often the terms “inhibitor” and “antagonists” can be used interchangeably.

As used herein, Kᵢ refers to a well known standard for “inhibition constant”, where “K” stands for constant and “i” stands for inhibition, and represents the point where 50% of the target (e.g., VEGFR) is inhibited. Kᵢ are determined by measuring enzyme activity in the presence of varying concentrations of test compound in assays such as those described, for example, in Example 6 and U.S. Pat. No. 6,316,306. Data can be analyzed using Enzyme Kinetic and Kaleidagraph software.

As used herein, the term “enhancer” or “enhance” (or variations thereof) refers to a ligand such as a compound or substance that improves, increases, stimulates, raises or positively interferes with a VEGFR activity. Often the terms “enhancer” or “agonists” can be used interchangeably. An enhancer would increase the enzyme's activity.

As used herein, the phrase “binding pocket,” also referred to as “binding site,” “ligand-binding site,” “catalytic domain,” or “ligand-binding pocket,” refers to a region or regions of a molecule or molecular complex, that, as a result of its shape, can associate with another chemical entity or compound. Such regions are useful in fields such as drug discovery. The association of natural ligands or synthetic ligands with binding pockets of their corresponding receptors is the basis of many biological mechanisms of action. Similarly, many drugs exert their biological effects via an interaction with the binding pockets of a receptor. Such interactions may occur with all or part of the binding pocket. An understanding of such interactions can facilitate the design of drugs having more favorable and specific interactions with their target receptor and thus, improved biological effects. Therefore, information related to ligand binding with a VEGFR2KD ligand-binding pocket is valuable in facilitating the design and discovery of modulators of othr VEGFRs and potentially other structurally related peptides. Furthermore, the more specificity in the design of a potential drug, the more likely that the drug will not interact with similar proteins, thus minimizing potential side effects due to unwanted cross interactions.

As used herein, a “VEGFRKD-like” peptide binding pocket refers to a peptide binding pocket defined by the atoms found in the structural coordinates as set forth in Table 1, Table 2, Table 3, Table 4 and/or Table 5, or defined by structural coordinates having a root mean square deviation of not more than about 0.90 Å from the binding pocket Cα atoms of any one of the VEGFR2 binding pockets set forth herein, or a conservatively substituted variant thereof. Preferably the root mean square deviation is not more than about 0.90 Å, more preferably not more than about 0.75 Å, and most preferably not more than about 0.60 Å.

As used herein, the term “activity” refers to all VEGFR activities, e.g. kinase activities, etc., as well as to the enzyme's potency. The terms “activity” and “function” are used interchangeably herein.

As used herein, the terms “model” and “modeling” mean the procedure of evaluating (also referred to as “assessing”) the affinity of the interaction between a VEGFRKD or VEGFRKD-like binding pocket and a chemical entity (also referred to as a “candidate compound”) based on, for example, steric constraints and surface/solvent electrostatic effects.

Nucleic Acids and Polynucleotides

Isolated nucleic acid molecules that encode members of the VEGFR family or proteins structurally related thereto are utilized in the present invention. More preferably, the nucleic acid molecules encode the kinase domain of a VEGFR or structurally related protein. Exemplary nucleic acid molecules encode the kinase domain of VEGFR2 or a modified VEGFR2.

Preferably, the nucleic acid molecules encode a modified, unphosphorylated VEGFR2 polypeptide wherein the kinase domain (KD) thereof has an amino acid sequence comprising SEQ ID NO: 3, as described in U.S. Pat. No. 6,316,603, which is incorporated by reference herein in its entirety. The modified VEGFR2KD contains a deletion of fifty amino acid residues when compared to the wild-type polypeptide kinase domain (see SEQ ID NO: 2). It is unlikely that this deletion significantly affects the conformation of the ligand binding site on the kinase. The kinetic phosphotransfer properties of the modified protein construct are similar to those of a construct containing the entire kinase insert domain, as expected, since the deleted section is a subdomain that is not necessary for catalytic phosphotransfer activity. The modified VEGFR2KD also contains one point mutation (E990V) compared to the wild-type VEGFR2 polypeptide (SEQ ID NO: 2). See U.S. Pat. No. 6,316,306; McTigue et al., Structure, Vol. 7, No. 3, pages 319-330 (1999).

The terms “nucleic acid molecule” and “polynucleotide” are used interchangeably in this application. These terms refer to any polyribonucleotide or polydeoxribonucleotide, which may be unmodified RNA or DNA or modified RNA or DNA. These terms are intended to include DNA molecules (e.g., cDNA) and RNA molecules (e.g., mRNA) and analogs of the DNA or RNA generated using nucleotide analogs. Exemplary polynucleotides include single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, double-stranded, or triple-stranded regions, or a mixture of single- and double-stranded regions. In addition, “polynucleotide” and “nucleic acid molecule” as used herein refer to triple-stranded regions composed of RNA or DNA, or both RNA and DNA. The strands in such regions may be from the same molecule or from different molecules. The regions may include all of one or more of the molecules, but more preferably involve only a region of some of the molecules. One of the molecules of a triple-helical region may be an oligonucleotide.

Exemplary polynucleotides and nucleic acid molecules also include DNAs or RNAs as described above that contain one or more modified bases. Moreover, DNAs or RNAs comprising unusual bases, such as inosine, or modified bases, such as tritylated bases are exemplary polynucleotides. Exemplary polynucleotides and nucleic acid molecules also include chemically, enzymatically or metabolically modified forms of polynucleotides, as well as the chemical forms of DNA and RNA characteristic of viruses and cells, including, for example, simple and complex cells. Exemplary polynucleotides also include short polynucleotides referred to as oligonucleotides.

As used herein, the term “isolated” nucleic acid molecule means that the material is free of proteins and other nucleic acid present in the natural environment in which the material is normally found. In particular, the nucleic acid molecule is free of cellular components. Exemplary isolated nucleic acid molecules include PCR products, mRNA, cDNA, or restriction fragments. In another embodiment, an isolated nucleic acid is preferably excised from the chromosome in which it may be found, and more preferably is no longer joined to non-regulatory, non-coding regions, or to other genes, located upstream or downstream of the gene in its natural environment in the chromosome. In yet another embodiment, the isolated nucleic acid lacks one or more introns. Isolated nucleic acid molecules can be inserted into plasmids, cosmids, artificial chromosomes, and the like. Thus, in a specific embodiment, a recombinant nucleic acid is an isolated nucleic acid. Moreover, an “isolated” nucleic acid molecule, such as a cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, a recombinant DNA molecule contained in a vector is considered isolated. Further examples of isolated DNA molecules include recombinant DNA molecules maintained in heterologous host cells or purified (partially or substantially) DNA molecules in solution. Exemplary isolated RNA molecules include in vivo or in vitro RNA transcripts of the isolated DNA molecules described herein. Exemplary isolated nucleic acid molecules further include such molecules produced synthetically.

Full-length genes or portions thereof may be cloned using any one of a number of suitable methods known in the art. For example, a method that employs XL-PCR (Perkin-Elmer, Foster City, Calif.) to amplify long pieces of DNA may be used.

The isolated nucleic acid molecules can encode functional polypeptides plus additional amino or carboxyl-terminal amino acids, such as those that, e.g., facilitate protein trafficking, prolong or shorten protein half-life, or facilitate manipulation of a protein for assay or production. Once a full-length gene is cloned, portions of the gene can be obtained using known techniques.

Isolated nucleic acid molecules can be in the form of RNA, such as mRNA, or in the form of DNA, including cDNA and genomic DNA, obtained by cloning or produced by known chemical synthetic techniques or by a combination thereof. The nucleic acid, especially DNA, can be double-stranded or single-stranded. Single-stranded nucleic acid can be the coding strand (sense strand) or the non-coding strand (antisense strand).

The invention further utilizes nucleic acid molecules that encode functional fragments or variants of VEGFRKDs. Such nucleic acid molecules may be constructed by known recombinant DNA methods or by chemical synthesis. Such non-naturally occurring variants may be made by mutagenesis techniques, including those applied to nucleic acid molecules, cells, or organisms. Accordingly, the variants can contain nucleotide substitutions, deletions, inversions and insertions. Variation can occur in either or both the coding and non-coding regions. The variations can produce both conservative and non-conservative amino acid substitutions.

The nucleic acid molecules utilized in the present invention are useful for producing peptides for use in crystallization studies, drug discovery, and drug design. The nucleic acid molecules can also be used as primers for PCR to amplify any given region of a nucleic acid molecule and are also useful to synthesize antisense molecules of desired length and sequence

The nucleic acid molecules are also useful for constructing recombinant vectors. Such vectors include expression vectors that express a portion of, or all of, the peptide sequences. Vectors also include insertion vectors, used to integrate into another nucleic acid molecule sequence, such as into the cellular genome, to alter in situ expression of a gene and/or gene product. For example, an endogenous coding sequence can be replaced via homologous recombination with all or part of the coding region containing one or more specifically introduced mutations.

The nucleic acid molecules are also useful for constructing host cells expressing a part, or all, of the nucleic acid molecules and peptides.

Vectors and Host Cells

The invention also utilizes vectors containing the nucleic acid molecules described above and, preferably, the modified VEGFR2KD described above and in U.S. Pat. No. 6,316,603 (incorporated by reference herein in its entirety).

When the vector is a nucleic acid molecule, the nucleic acid molecules are covalently linked to the vector nucleic acid. Exemplary vectors for this embodiment of the invention include plasmids, single- or double-stranded phage, single- or double-stranded RNA or DNA viral vector, or artificial chromosome, such as a BAC, PAC, YAC, or MAC. Various expression vectors can be used to express the polynucleotides of the invention, such as pET and pProEX.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

The vectors can be used for the maintenance (cloning vectors) or expression (expression vectors) of the nucleic acid molecules. The vectors can function in prokaryotic or eukaryotic cells or in both (shuttle vectors).

Expression vectors contain cis-acting regulatory regions that are operably linked in the vector to the nucleic acid molecules such that transcription of the nucleic acid molecules is allowed in a host cell. The nucleic acid molecules can be introduced into the host cell with a separate nucleic acid molecule capable of affecting transcription. Thus, the second nucleic acid molecule may provide a trans-acting factor interacting with the cis-regulatory control region to allow transcription of the nucleic acid molecules from the vector. Alternatively, the host cell may supply a trans-acting factor. Finally, a trans-acting factor can be produced from the vector itself. It is understood, however, that in some embodiments, transcription and/or translation of the nucleic acid molecules can occur in a cell-free system.

Exemplary regulatory sequences to which the nucleic acid molecules used herein can be operably linked include promoters for directing mRNA transcription. These include the left promoter from bacteriophage λ, the lac promoter, TRP, and TAC promoters from E. coli, the early and late promoters from SV40, the CMV immediate early promoter, the adenovirus early and late promoters, and retrovirus long-terminal repeats.

The term “operably linked” as used herein indicates that a gene and a regulatory sequence, such as a promoter, are connected in such a way as to permit gene expression when the appropriate molecules (e.g., transcriptional activator proteins or proteins which include transcriptional activation domains) are bound to the regulatory sequence.

In addition to control regions that promote transcription, exemplary expression vectors also include regions that modulate transcription, such as repressor binding sites and enhancers. Illustrative embodiments include the SV40 enhancer, the cytomegalovirus immediate early enhancer, polyoma enhancer, adenovirus enhancers, and retrovirus LTR enhancers.

In addition to containing sites for transcription initiation and control, exemplary expression vectors can contain sequences necessary for transcription termination. These vectors may also contain signals necessary for translation such as a ribosome-binding site. Other exemplary regulatory control elements for expression include initiation and termination codons as well as polyadenylation signals. Other examples of regulatory sequences are described, for example, in Sambrook et al., 2001, supra.

A variety of expression vectors can be used to express a nucleic acid molecule. Examples of such vectors include chromosomal, episomal, and virus-derived vectors, for example, vectors derived from bacterial plasmids, from bacteriophage, from yeast episomes, from yeast chromosomal elements, including yeast artificial chromosomes, and from viruses such as baculoviruses, papovaviruses such as SV40, vaccinia viruses, adenoviruses, poxviruses, pseudorabies viruses, and retroviruses. Vectors may also be derived from combinations of these sources, such as those derived from plasmid and bacteriophage genetic elements, e.g., cosmids and phagemids. Appropriate cloning and expression vectors for prokaryotic and eukaryotic hosts are described in Sambrook et al., 2001, supra.

The regulatory sequence may provide constitutive expression in one or more host cells (i.e. tissue specific) or may provide for inducible expression in one or more cell types such as by temperature, nutrient additive, or exogenous factor such as a hormone or other ligand. Suitable vectors providing for constitutive and inducible expression in prokaryotic and eukaryotic hosts are known in the art.

The nucleic acid molecules can be inserted into the vector nucleic acid by known methodology. For example, the DNA of interest is joined to a vector by cleaving the DNA sequence and the vector with one or more restriction enzymes and then ligating the fragments together.

The vector containing the appropriate nucleic acid molecule can be introduced into an appropriate host cell for propagation or expression using known techniques. Appropriate bacterial host cells include E. coli, Streptomyces, and Salmonella typhimurium. Appropriate eukaryotic host cells include yeast, insect cells, animal cells such as COS and CHO, and plant cells.

The peptide as described herein can be expressed as a fusion protein. Fusion vectors can increase the expression of a recombinant protein, increase the solubility of the recombinant protein, and/or aid in the purification of the protein by acting, for example, as a ligand for affinity purification. A proteolytic cleavage site may be introduced at the junction of the fusion moiety so that the desired peptide can ultimately be separated from the fusion moiety. Exemplary proteolytic enzymes include factor Xa, thrombin, and enterokinase. Illustrative fusion expression vectors include pGEX (Smith et al., Gene 67:31-40 (1988)), pET28a (Novagen, Madison, Wis.), pMAL (New England Biolabs, Beverly, Mass.), and pRIT5 (Pharmacia, Piscataway, N.J.), which fuse glutathione S-transferase (GST), maltose E binding protein, or protein A, respectively, to the target recombinant protein. Examples of suitable inducible non-fusion E. coli expression vectors include pTrc (Amann et al., Gene 69:301-315 (1988)) and pET 11d (Studier et al., Gene Expression Technology: Methods in Enzymology, 185:60-89 (1990)).

Recombinant protein expression can be maximized in a host bacteria by providing a genetic background wherein the host cell has an impaired capacity to proteolytically cleave the recombinant protein. (Gottesman, Gene Expression Technology: Methods in Enzymology, 185:119-128 (1990)). Alternatively, the sequence of the nucleic acid molecule of interest can be altered to provide preferential codon usage for a specific host cell, for example, E. coli. (Wada et al., Nucleic Acids Res. 20:2111-2118 (1992)).

The nucleic acid molecules can also be expressed by expression vectors that are operative in yeast. Examples of vectors for expression in yeast, e.g. S. cerevisiae, include pYepSec1 (Baldari, et al., EMBO J. 6:229-234 (1987)), pMFa (Kurjan et al., Cell 30:933-943 (1982)), pJRY88 (Schultz et al., Gene 54:113-123 (1987)), and pYES2 (Invitrogen Corporation, San Diego, Calif.).

The nucleic acid molecules can also be expressed in insect cells using, for example, baculovirus expression vectors. Exemplary, baculovirus vectors available for expression of proteins in cultured insect cells (e.g., Sf 9 cells) include the pAc series (Smith et al., Mol. Cell Biol. 3:2156-2165 (1983)) and the pVL series (Lucklow et al., Virology 170:31-39 (1989)).

The nucleic acid molecules described herein can be expressed in mammalian cells using mammalian expression vectors. Examples of mammalian expression vectors include pCDM8 (Seed, Nature 329:840 (1987)) and pMT2PC (Kaufman et al., EMBO J. 6:187-195 (1987)).

Expression vectors include, for example, pET28a (Novagen, Madison, Wis.), pAcSG2 (Pharmingen, San Diego, Calif.), pProEx (Life Technologies, Gaithersburg, Md.) and pFastBac (Life Technologies). Other vectors suitable for maintenance propagation or expression of the nucleic acid molecules described herein are known in the art. For example, suitable vectors and methods for using and propagating vectors are discussed in Sambrook et al., 2001, supra.

Exemplary host cells containing the vectors used herein include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques available in the art. These include calcium phosphate transfection, DEAE-dextran-mediated transfection, cationic lipid-mediated transfection, electroporation, transduction, infection, lipofection. See also, Sambrook et al., 2001, supra.

The recombinant host cells expressing the peptides described herein have a variety of uses. For example, the cells are useful for producing the polypeptides of the invention, which can be used for crystallography studies, biochemical studies, and drug discovery.

Host cells can contain more than one vector. Thus, different nucleotide sequences can be introduced on different vectors of the same cell. Similarly, the nucleic acid molecules can be introduced either alone or with other nucleic acid molecules that are not related to the nucleic acid molecules, such as those providing trans-acting factors for expression vectors. When more than one vector is introduced into a cell, the vectors can be introduced independently, co-introduced, or joined to the VEGFRKD polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Exemplary vectors include selectable markers that enable the selection of the subpopulation of cells that contain the recombinant vector constructs. The marker can be contained in the same vector that contains the nucleic acid molecules described herein or may be on a separate vector. Exemplary markers include tetracycline or ampicillin-resistance genes for prokaryotic host cells, and dihydrofolate reductase or neomycin resistance for eukaryotic host cells. However, any marker that provides selection for a phenotypic trait may be used.

Peptides, Proteins and Antibodies

The crystalline structures of the invention comprise the protein construct as described in U.S. Pat. No. 6,316,603 B1 (incorporated herein in its entirety) and McTigue et al., Structure, Vol. 7, No. 3, 319-330 (1999). The protein construct, referred to herein as VEGFR2KD (SEQ ID NO: 3), contains the core kinase domain of VEGFR2 (SEQ ID NO: 2), with residues 940-989 of the kinase insert domain of SEQ ID NO: 2, a subdomain not necessary for catalytic phosphotransfer activity, deleted. The kinetic phosphotransfer properties of this protein construct are similar to those of a construct containing the entire kinase insert domain (See McTigue et al. Structure, Vol. 7, No. 3, 319-330 (1999); U.S. Pat. No. 6,316,603 B1). The methods used to isolate and purify the VEGFR2KD protein are described in McTigue et al. Structure, Vol. 7, No. 3, 319-330 (1999) and U.S. Pat. No. 6,316,603 B1 (incorporated herein in their entireties by reference).

The following amino acid abbreviations are used herein: a=A=Ala=Alanine; v=V=Val=Valine; I=L=Leu=Leucine; I=I=Ile=Isoleucine; p=P=Pro=Proline; f=F=Phe=Phenylalanine; w=W=Trp=Tryptophan; m=M=Met=Methionine; g=G=Gly=Glycine; s=S=Ser=Serine; t=T=Thr=Threonine; c=C=Cys=Cysteine; y=Y=Tyr=Tyrosine; n=N=Asn=Asparagine; q=Q=Gln=Glutamine; d=D=Asp=Aspartic Acid; e=E=Glu=Glutamic Acid; k=K=Lys=Lysine; r=R=Arg=Arginine; and h=H=His=Histidine.

As used herein, the term “polypeptide” refers to any peptide comprising two or more amino acids joined to each other by peptide bonds or modified peptide bonds (i.e., peptide isosteres). “Polypeptide” refers to both short chains, generally referred to as peptides, oligopeptides, or oligomers, and to long chains, generally referred to as proteins. The terms “peptide,” “polypeptide,” and “protein” are used interchangeably herein.

As used herein, a peptide is said to be “isolated” or “purified” when it is substantially free of cellular material or chemical precursors or other chemicals. The peptides of the present invention can be purified to homogeneity or other degrees of purity. The level of purification will be selected based on the intended use, such that the preparation allows for the desired function of the peptide, even if in the presence of considerable amounts of other components.

The phrase “substantially free of cellular material” means preparations of the peptide having less than about 30% (by dry weight) other proteins (i.e., contaminating protein). In preferred embodiments, the peptide preparation contains less than about 20% other proteins, more preferably, less than about 10% other proteins, or even more preferably, less than about 5% other proteins. When the peptide is recombinantly produced, it can also be substantially free of culture medium (i.e., culture medium represents less than about 20% of the volume of the protein preparation).

The phrase “substantially free of chemical precursors or other chemicals” refers to preparations of the peptide in which it is separated from chemical precursors or other chemicals that are involved in its synthesis. The phrase means preparations of the VEGFR polypeptide or VEGFR-related polypeptide having less than about 30% (by dry weight) chemical precursors or other chemicals. In preferred embodiments, the peptide preparations have less than about 20% chemical precursors or other chemicals, more preferably, less than about 10% chemical precursors or other chemicals, and, even more preferably, less than about 5% chemical precursors or other chemicals.

The isolated VEGFR polypeptides described herein can be purified from cells that have been altered to express the polypeptide (recombination), or synthesized using known protein synthesis techniques. For example, a nucleic acid molecule encoding VEGFR2 may be cloned into an expression vector, the expression vector introduced into host cells, and the protein expressed in the host cells. The protein can then be isolated from the cells by an appropriate purification scheme using standard protein purification techniques.

While the polypeptides of the invention can be produced in bacteria, yeast, mammalian cells, and other cells under the control of the appropriate regulatory sequences, cell-free transcription and translation systems can also be used to produce these proteins using RNA derived from the DNA constructs described herein. Where secretion of the peptide is desired, appropriate secretion signals are incorporated into the vector. The signal sequence can be endogenous or heterologous to the peptides. It is also understood that, depending upon the host cell in recombinant production of the peptides described herein, the peptides can have various glycosylation patterns, depending upon the cell, or non-glycosylated, as when produced in bacteria. In some embodiments, the peptides may contain an initial modified methionine as a result of a host-mediated process.

The present invention also provides the use of variants of the above-described peptides, such as allelic/sequence variants of the peptides, and non-naturally occurring recombinantly derived variants of the peptides. Such variants can be generated using techniques that are known by those skilled in the fields of recombinant nucleic acid technology and protein biochemistry.

Such variants can readily be made or identified using molecular techniques and the sequence information disclosed herein. Further, such variants can readily be distinguished from other peptides based on sequence and/or structural homology to the peptides of the present invention.

To determine the percent identity of two amino acid sequences or two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably 40%, more preferably 50%, even more preferably 60% or more, of the length of the reference sequence. In a preferred embodiment, the length of a reference sequence aligned for comparison purposes is at least 70%, preferably 80%, more preferably 90% or more, of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Lesk, ed., “Computational Molecular Biology” (1988) Oxford University Press, New York; Smith, ed., “Biocomputing: Informatics and Genome Projects” (1993) Academic Press, New York; Griffin et al., eds., “Computer Analysis of Sequence Data, Part 1” (1994) Humana Press, New Jersey; von Heinje, “Sequence Analysis in Molecular Biology” (1987) Academic Press; and Gribskov et al. eds., “Sequence Analysis Primer” (1991) Stockton Press, New York). For example, the percent identity between two amino acid sequences is determined using the Needleman et al. algorithm (J. Mol. Biol. 48:444-453 (1970), which has been incorporated into commercially available computer programs, such as GAP in the GCG software package, using either a Blossom 62 matrix or a PAM250 matrix, and a gap weight of 16, 14, 12, 10, 8, 6, or 4 and a length weight of 1, 2, 3, 4, 5, or 6. The percent identity between two nucleotide sequences can also be determined using the commercially available computer programs including the GAP program in the GCG software package (Devereux et al., Nucleic Acids Res. 12(1):387 (1984)), the NWS gap DNA CMP matrix and a gap weight of 40, 50, 60, 70, or 80 and a length weight of 1, 2, 3, 4, 5, or 6. The percent identity between two amino acid or nucleotide sequences can be determined using the algorithm of Meyers et al. (CABIOS, 4:11-17 (1989)), which has been incorporated into commercially available computer programs, such as ALIGN (version 2.0), using a PAM120 weight residue table, a gap length penalty of 12 and a gap penalty of 4.

The nucleic acid and protein sequences used in the present invention can further be used as a “query sequence” to perform a search against sequence databases to, for example, identify other family members or related sequences. Such searches can be performed using commercially available search engines, such as the NBLAST and XBLAST programs (version 2.0) of Altschul et al. (J. Mol. Biol. 215:403-10 (1990)). Nucleotide searches can be performed with such programs to obtain nucleotide sequences homologous to the nucleic acid molecules of the invention. Protein searches can be performed with such programs to obtain amino acid sequences homologous to the proteins of the invention. To obtain gapped alignments for comparison purposes, Gapped BLAST can be utilized as described in Altschul et al. (Nucleic Acids Res. 25(17):3389-3402 (1997)).

Peptides can be routinely identified as having a high degree (significant) of sequence homology/identity to the peptides of the present invention. As used herein, two proteins (or a region of the proteins) have “significant homology” when the amino acid sequences are typically at least about 70-75% homologous. In preferred embodiments, the homology is 80-85%, and more preferably at least about 90-95%. A significantly homologous amino acid sequence will be encoded by a nucleic acid sequence that will hybridize to a peptide encoding nucleic acid molecule under stringent conditions.

Non-naturally occurring variants of the polypeptides used in the present invention can be generated using recombinant techniques. Such variants include deletions, additions and substitutions in the amino acid sequence in the kinase domain. For example, one class of substitutions are conservative amino acid substitutions. Such substitutions are those that substitute a given amino acid in a peptide by another amino acid of like characteristics. Exemplary conservative substitutions are the replacements, one for another, among the aliphatic amino acids (Ala, Val, Leu, and Ile); interchange of amino acids containing a hydroxyl residue (Ser and Thr); exchange of amino acids containing an acidic residue (Asp and Glu); substitution between amino acids containing an amide residue (Asn and Gln); exchange of amino acids containing a basic residue (Lys and Arg); and replacements among amino acids containing an aromatic residue (Phe, Tyr). Guidance concerning which amino acid changes are likely to be phenotypically silent is found in Bowie et al., Science 247:1306-1310 (1990).

Variant peptides can be fully functional or may have reduced or decreased activity when compared to the wild-type protein. Fully functional variants may contain conservative variation or variation in non-critical residues or in non-critical regions. Functional variants can also contain substitution of similar amino acids, not affecting function that result in no change or an insignificant change in function. Alternatively, such substitutions may positively or negatively affect function to some degree.

Exemplary non-functional variants are those having one or more non-conservative amino acid substitutions, deletions, insertions, inversions, or truncations of the particular polypeptide, or a substitution, insertion, inversion, or deletion in a critical residue or critical region of the polypeptide.

Amino acids that affect function can be identified by methods known in the art, such as site-directed mutagenesis or alanine-scanning mutagenesis (Cunningham et al., 1989, Science 244:1081-1085). The latter procedure introduces single alanine mutations at every residue in the molecule. The resulting mutant molecules are then tested for biological activity, for example, by measuring enzymatic activity. Sites that are critical for binding can also be determined by structural analysis, such as by X-ray crystallography, nuclear magnetic resonance, or photoaffinity labeling (Smith et al., J. Mol. Biol. 224:899-904 (1992); de Vos et al., Science 255:306-312 (1992)). Accordingly, the peptides of the present invention also include derivatives or analogs: in which a substituted amino acid residue is not one encoded by the genetic code; in which a substituent group is included; in which the polypeptide is fused with another compound, such as a compound to increase the half-life of the polypeptide (for example, polyethylene glycol); or in which the additional amino acids are fused to the polypeptide, such as a leader or secretory sequence or a sequence for purification of the polypeptide.

The present invention further provides for use of functional, active fragments of the VEGFR2KD. A “fragment” is a variant polypeptide having an amino acid sequence that is entirely the same as part but not all of any amino acid sequence of any polypeptide used in the invention. Fragments may be free-standing or comprised within a larger polypeptide of which they form a part or region; most preferably they are a single continuous region in a single larger polypeptide. As used herein, a “fragment” comprises at least 8 or more contiguous amino acid residues from the protein binding domain. Such fragments can be chosen based on the ability to retain the biological activity of the binding domain or based on the ability to perform a function, e.g., act as an immunogen. Preferred are fragments that are active and that have improved crystallography properties as compared to the modified VEGFR2KD used herein.

Polypeptides may contain amino acids other than the 20 amino acids commonly referred to as the 20 naturally occurring amino acids. Further, many amino acids, including the terminal amino acids, may be modified by natural processes, such as byprocessing and other post-translational modifications, or by chemical modification techniques known in the art. Known modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, phenylation, racemization, selenoylation, sulfation, transfer-RNA mediated addition of amino acids to proteins such as arginylation, and ubiquitination. Modifications, such as glycosylation, lipid attachment, sulfation, gamma-carboxylation of glutamic acid residues, hydroxylation and ADP-ribosylation, for instance, are described in most basic texts, such as Creighton, “Proteins-Structure and Molecular Properties,” 2nd ed. (1993) W. H. Freeman and Company, New York. Reviews on this subject include Wold, “Posttranslational Covalent Modification of Proteins,” Johnson, ed., Academic Press, New York 1-12 (1983); Seifter et al. (Meth. Enzymol. 182: 626-646 (1990)); and Rattan et al. (Ann. N.Y. Acad. Sci. 663:48-62 (1992)).

In some embodiments, the peptides can be attached to heterologous sequences to form chimeric or fusion proteins. Such chimeric and fusion proteins comprise a peptide operatively linked to a heterologous protein having an amino acid sequence not substantially homologous to the VEGFR peptide. “Operatively linked” indicates that the peptide and the heterologous protein are fused in-frame. The heterologous protein can be fused to the N-terminus or C-terminus of the VEGFR peptide. The two peptides linked in a fusion peptide are preferrably derived from two independent sources, and therefore such a fusion peptide comprises two linked peptides not normally found linked in nature.

In some embodiments, the fusion protein does not affect the activity of the peptide per se. For example, the fusion protein can include, enzymatic fusion proteins or affinity tags, for example, beta-galactosidase fusions, yeast two-hybrid GAL fusions, His-tags, MYC-tags, green fusion protein, and Ig fusions. Such fusion proteins can facilitate the purification of the polypeptides described herein. In certain host cells (e.g., mammalian host cells), expression and/or secretion of a protein can be increased by using a heterologous signal sequence.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques, including automated DNA synthesizers. Alternatively, PCR amplification of gene fragments can be carried out using anchor primers which give rise to complementary overhangs between two consecutive gene fragments, which can subsequently be annealed and re-amplified to generate a chimeric gene sequence (see Ausubel et al., 1992 supra). Moreover, many expression vectors are commercially available that already encode a fusion moiety (e.g., a GST protein, His-tag, or green fluorescent protein). A nucleic acid encoding a VEGFR polypeptide can be cloned into such an expression vector such that the fusion moiety is linked in-frame to the VEGFR polypeptide.

The polypeptides can be used for rapid-screening methods (high-throughput screening) to identify compounds that inhibit or modulate VEGFR activity. The high-throughput screening assay can be fully automated on robotic workstations. The assay may employ radioactivity, fluorescence, or other materials useful for detection.

“High-throughput screening” as used herein refers to an assay that provides for multiple-candidate agents or samples to be screened simultaneously. Preferably the number of agents or samples screened is greater than one, more preferably greater than 100, and even more preferably greater than 300. Such assays may include the use of microtiter plates or other vessel containing apparatus that allows a large number of assays to be carried out simultaneously, using small amounts of reagents and samples.

Crystallization

The present invention provides methods for growing mammalian, e.g., human, VEGFRKD crystals, including, but not limited to, unphosphorylated VEGFR2KD:ligand crystalline complexes. Crystals of the VEGFRKD:ligand can be grown by a number of known techniques, including, but not limited to, batch crystallization, vapor diffusion (either by sitting drop or hanging drop), and microdialysis. See, e.g., McPherson, A., Preparation and Analysis of Protein Crystals, Krieger Press (1989). Seeding of the crystals may be required to obtain X-ray quality crystals. If seeding is required, then standard micro and/or macro seeding of crystals may be employed.

Preferably, crystallization is performed by hanging-drop vapor diffusion wherein, for example, a droplet of VEGFR2KD solution is mixed with a droplet of precipitant solution to obtain a mixed droplet solution. The mixed droplet solution is then suspended over a well of precipitant solution in a sealed container. The mixed droplet solution is preferably placed on a glass slide prior to inclusion in the sealed container. In a preferred embodiment, the VEGFR2KD solution is mixed with the precipitant solution in a ratio ranging from about 1:4 to about 4:1, preferably, ranging from about 1:2 to about 2:1 and, even more preferably, of about 1:1. In one embodiment, the mixed droplet may be suspended over a well containing precipitant solution.

The vapor pressure of the precipitant solution in the well must be lower than the vapor pressure of the mixed droplet solution in order for crystals to form. The crystallization temperature may be between about 4° C. and about 20° C., and, preferably, is about 4° C. The mixed droplet solution is allowed to stand suspended over the well containing the precipitant solution at the crystallization temperature for a period of about 12 to about 24 hours, preferably, about 12 hours. The seal on the crystallization experiment is then opened and the drop is seeded with micro or macro seeds that are not older than about 28 days. The crystallization experiment is resealed and the crystals are allowed to grow for a period of about 1 to about 4 weeks, preferably until the crystals reach a size appropriate for crystallographic data collection, such as about 0.1 mm×about 0.1 mm×about 0.25 mm. Preferably, the ligand comprises a VEGFRKD modulator (e.g. activity inhibitor or enhancer) which binds to the KD ligand binding pocket. In a preferred embodiment, the ligand is selected from the group consisting of: 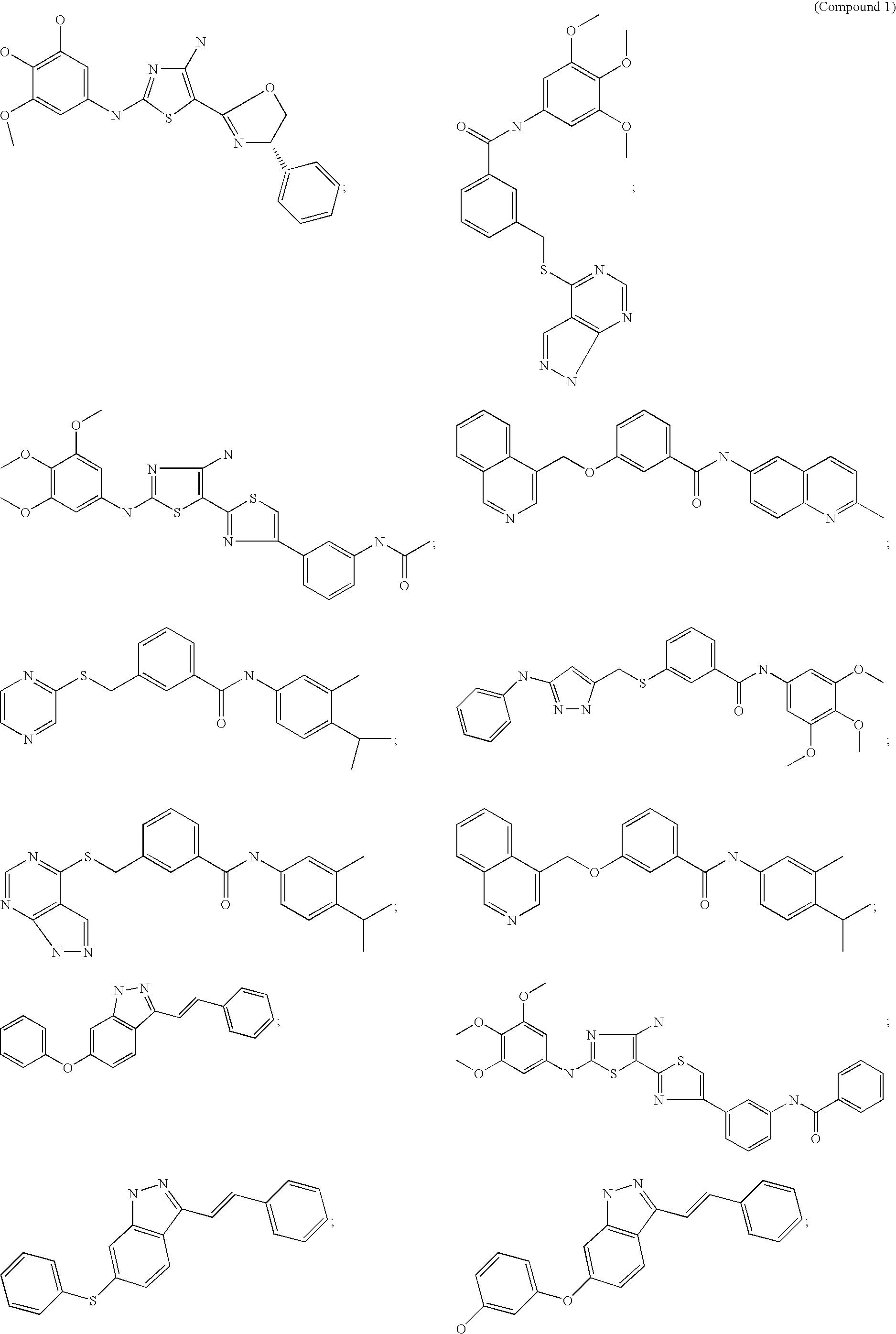 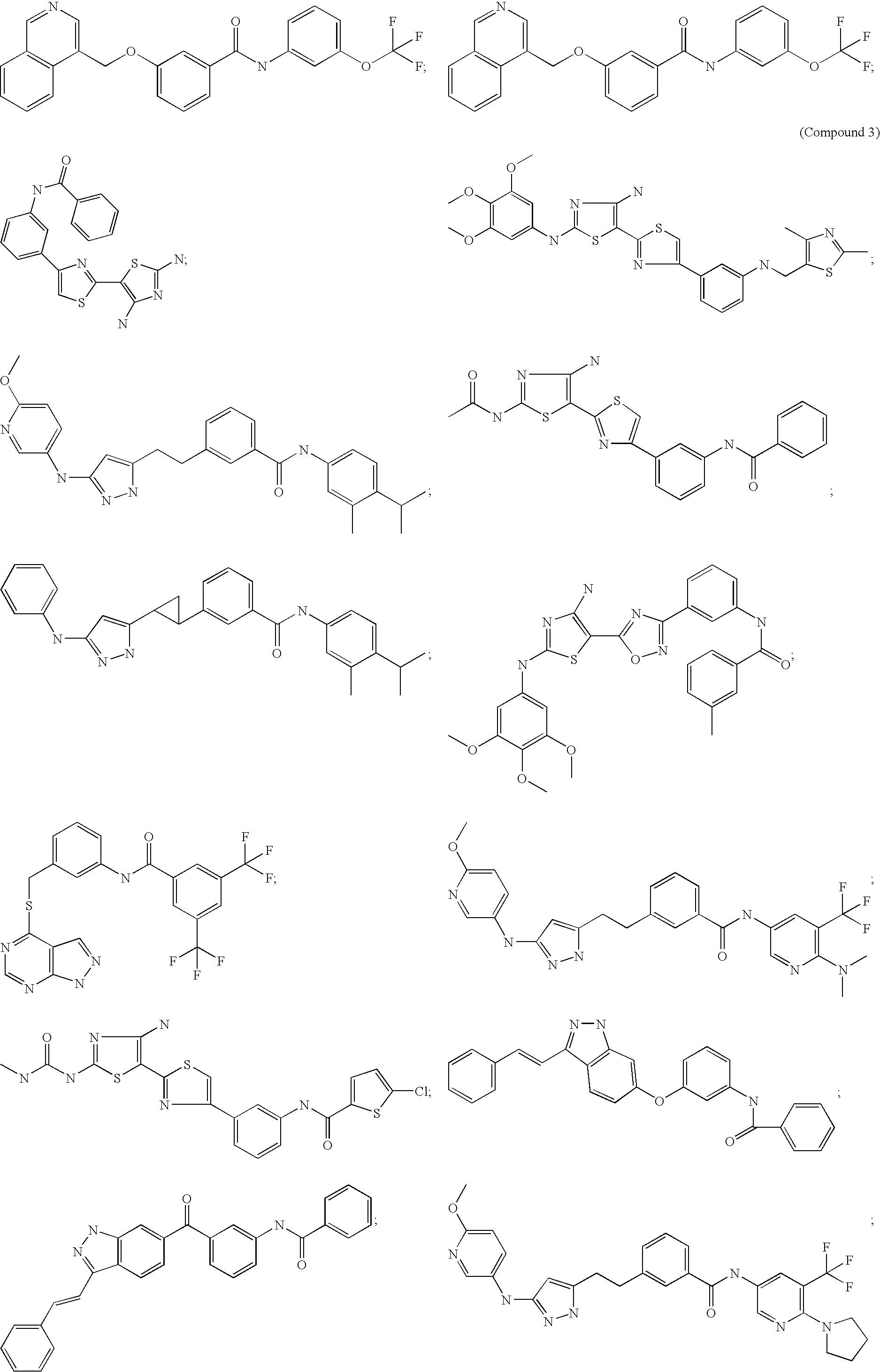 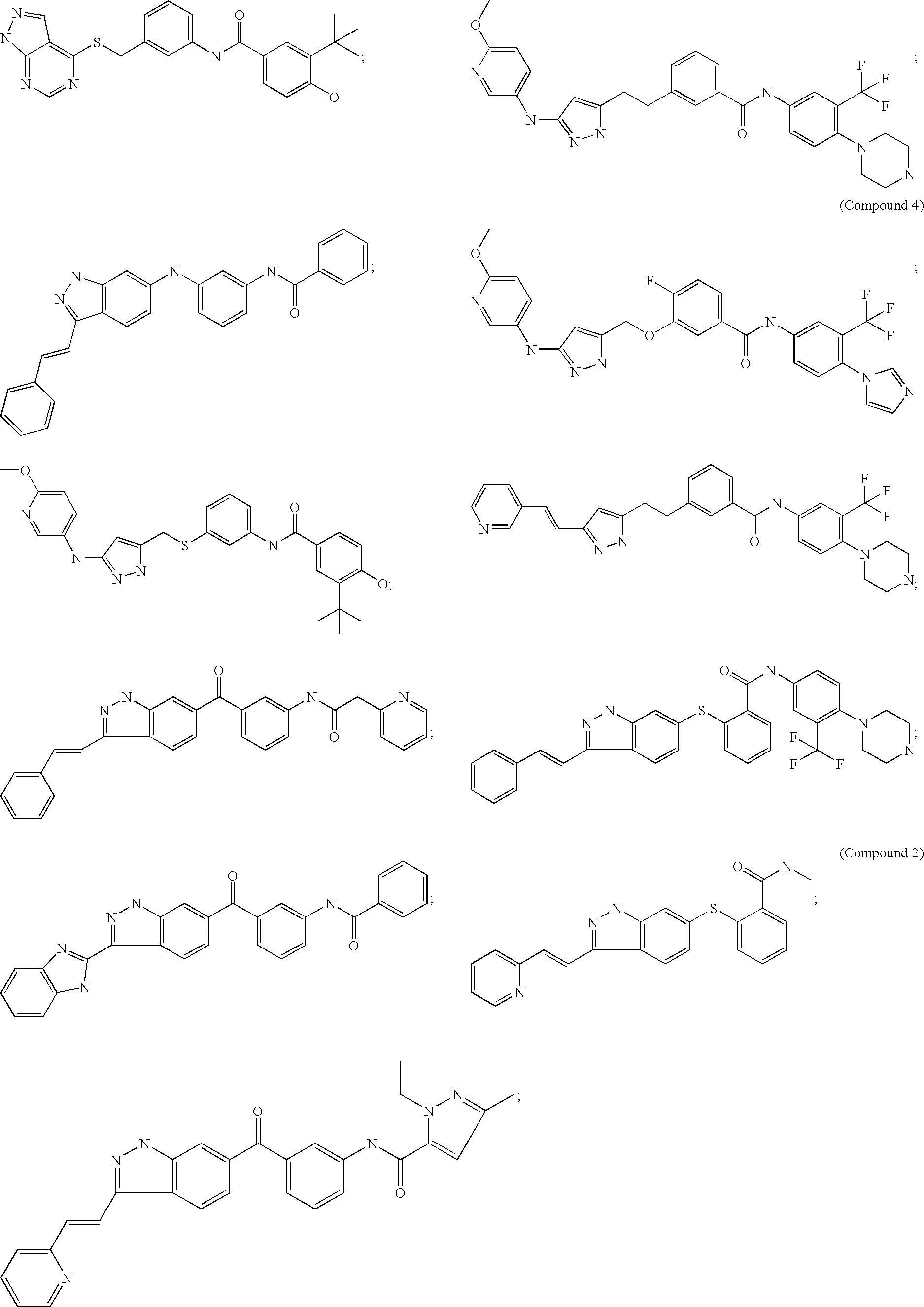 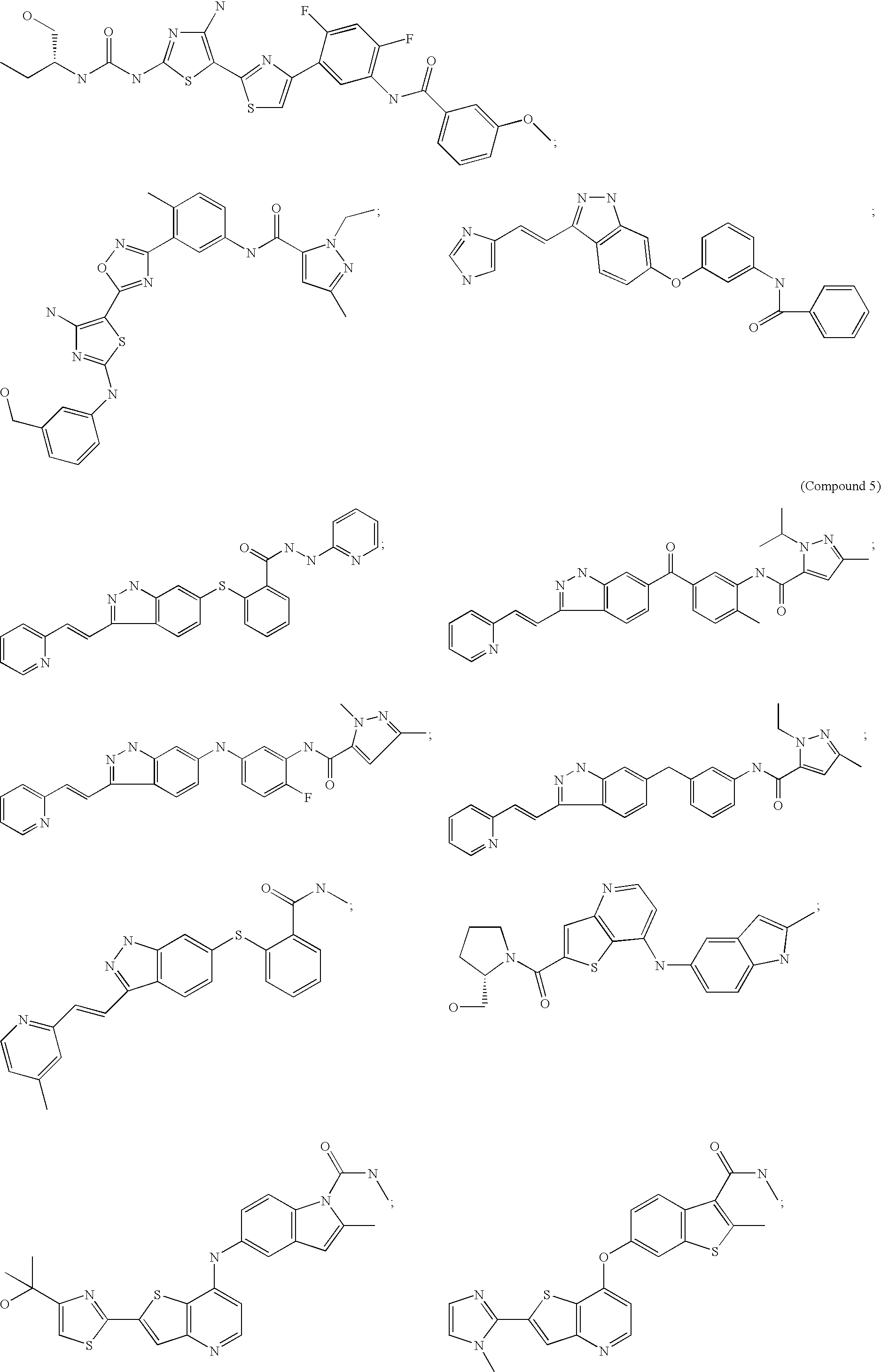 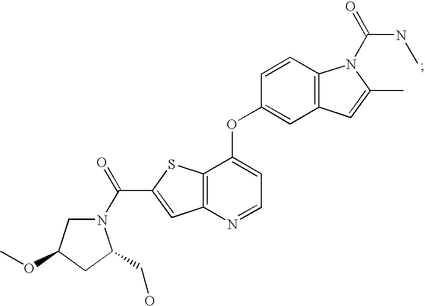
and mixtures thereof, and compounds related thereto. (See, e.g., WO 01/02359 A2, incorporated herein by reference). The crystalline structures of VEGFR2KD in complex with these ligands are defined by the atomic coordinates set forth in Tables 1, Table 2, Table 3, Table 4, and/or Table 5, or by related structural (e.g. atomic) coordinates having a root mean square deviation of not more than about 1.25 Å from the Cα atoms of the structural coordinates a set forth in Table 1, Table 2, Table 3, Table 4, and/or Table 5.

In a more preferred embodiment, the ligand is Compound 1, Compound 2, Compound 3, Compound 4 or Compound 5.

In the most preferred embodiment, the ligand is Compound 2 (Kᵢ=3.8 nM, measured using unphosphorylated VEGFR2KD in the coupled Spectrophotometric Enzyme Assay described in Example 6).

The VEGFRKD:ligand crystals are harvested and dipped in a cryoprotective solution. The cryoprotective solution comprises components designed to stabilize the formation of a vitreous solid containing the VEGFRKD complex as a crystalline solid at a temperature of about 100° K. The solution is then flash-frozen by immersion in a stream of cold nitrogen at, for example, 100° K. Alternatively, the crystals may be dipped directly into liquid nitrogen or liquid propane.

Crystals of the present invention may take a variety of forms, all of which are included in the present invention, such as triclinic, monoclinic, orthorhombic, tetragonal, cubic, trigonal or hexagonal. In a preferred embodiment, the crystals of human VEGFR2KD are monoclinic and possess a space group C2 with unit cell dimensions of approximately: a=136 Å, b=57 Å and c=52 Å, alpha=90°, beta−94° and gamma=900.

X-Ray Diffraction Data and Structural Analysis

Once crystals of the present invention are grown, X-ray diffraction data can be collected. The data collection methods and conditions cited herein are provided to elucidate the approach used for the structural determination of VEGFR2KD peptide complexes. One of ordinary skill in the art would be aware of other methods and conditions that may be suitable for X-ray data collection and structural determination of VEGFR2KD peptide complexes. See, e.g., Glusker, J., Crystal Structure Analysis for Chemists and Biologists, Wiley-VCH Press (1994).

Generally, collecting the X-ray diffraction data for the VEGFR2KD peptide complex crystals comprises mounting the crystals in a cryoloop, bathing the crystals in a cryoprotectant solution and rapidly cooling the crystals to about 100 K, followed by collecting diffraction data in the oscillation mode. The source(s) of X-rays includes, but is not limited to, a standard rotating anode home source, such as a Rigaku™ Ru-H3R or Ru-200B generator (Rigaku Corp., Tokyo, Japan), a sealed tube or a synchrotron source, such as a synchrotron provided by, for example, the Stanford University Synchrotron Radiation Laboratory.

The method of detecting and quantitating the diffraction data (i.e., diffraction pattern produced by the diffracted X-rays), may be performed using, for example, a standard image plate, such as the R-Axis IV⁺⁺ (Rigaku/MSC, Inc., The Woodlands, Tex.), a MAR300 or MAR345i (MAR Research San Diego, Calif.) or a charge-coupled device such as the MAR-CCD X-ray detector. The data are generally corrected for Lorentz and polarization effects and converted to indexed structure factor amplitudes using data processing software such as DENZO™, HKL-2000 or SCALEPACK (HKL Research, Inc., Charlottesville, Va.) (Otwinowski, Z. et al., Methods Enzymol. 276: 307-326 (1997)), d*Trek (Rigaku/MSC, Inc. (Pflugrath, J. W., Acta Cryst. D55: 1718-1725 (1999))), or MOSFILM (Leslie, A. G. W., Joint CCP4+ESF-EAMCB Newsletter on Protein Crystallography, No. 26 (1992)), or other functionally related computer programs, alone or in combination. The preferred processing software is DENZO™ and/or HKL-2000 (HKL Research, Inc.). In addition, X-PLOR (Brunger, “X-PLOR: A System for X-ray Crystallography and NMR,” Yale University Press, New Haven, Conn. (1992)) or Heavy (Terwilliger, Los Alamos National Laboratory) may be utilized for bulk solvent correction and B-factor scaling.

The three-dimensional image generated from the X-ray diffraction data, more specifically, generated from the intensities of the diffracted X-rays, is referred to as an electron density map of the repeating unit of the crystal. However, the electron density map cannot be completely generated until the amplitudes and phases of the diffracted X-rays are known. Amplitudes may be obtained directly from the intensities. Phases may be obtained indirectly by, for example, any one or a combination of the following methods: computational methods, molecular replacement analysis (if a homologous structure is known), heavy atom substitution techniques (e.g., isomorphous replacement), synchrotron radiation at multiple wavelengths, Patterson difference, single-wavelength anomalous scattering, etc. Software that can aid in generating the electron density map includes, but is not limited to, SHARP (Statistical Heavy Atom Refinement and Phasing) (de la Fortelle, E. et al., Meth. Enzymol. 276: 472-494 (1997)) and SOLOMON (Abrahams, J. P. et al., Acta Cryst. D52: 30-42 (1996)), and other related computer program, alone or in combination. The map may then be used, via model building, to build a model of the protein. A molecular model of the amino acid or nucleotide sequence is then fit into the electron density map and the map is refined. Refinement establishes a set of atomic coordinates representing every non-hydrogen molecule of the enzyme or enzyme complex and results in a three-dimensional structure. Atomic coordinates (also referred to as “structure coordinates,” “structural coordinates” and “crystal coordinates”) are Cartesian coordinates derived from mathematical equations related to the patterns obtained on diffraction of a monochromatic beam of X-rays by the atoms of a protein or protein complex in crystal form.

Molecular models can be built into the electron density map and refined using, for example, O (Jones et al., ACTA Crystallogr. A47:110-119 (1991)), XTALVIEW (Scripps Research, La Jolla, Calif.), QUANTA98 (Accelrys, Inc., San Diego, Calif.), GRIN/GRID (Molecular Discovery Ltd., London, England), MolCad (Tripos, Inc.), CHARMm™ (Accelrys Inc.), INSIGHT™-II (Accelrys Inc.), SYBYL (Tripos, Inc.), MacroModel™ (Trustees of Columbia Univ., New York, N.Y.), ICM (MolSoft LLC, San Diego, Calif.), CNX (Accelrys Inc.), CAVEAT (P. A. Bartlett, et al., Royal Chem. Soc. 78: 182-196 (1989), available from the University of California, Berkley, Calif.), GRASP (A. Nicholls, Columbia University), SiteID (Tripos, Inc.), and/or X-PLOR (Accelrys Inc.), using the free R-value to monitor the course of refinement, or other functionally related computer program. Any of the programs may be used individually or in combination.

The computer software may be used alone or combined with a docking computer program such as GOLD (commercially available via Cambridge Crystallographic Data Centre, Cambridge, UK; Jones, G., J. Mol. Biol. 245: 43-53 (1995)), FlexX (Tripos, Inc.), GRAMM (Ilya A. Vakser, Rockefeller Univ.), Flexidock (Tripos, Inc.), Dock (Ewing, T. J. A. et al., J. Comput-Aided Mol. Des. 15: 411-428 (2001)), or AutoDock (Molecular Graphics Laboratory (Scripps Research Inst.); Goodsell, D. S., J. Mol. Recognit. 9: 1-5 (1996)), or other functionally related computer program. These docking computer programs scan known databases of small molecules to find core compounds that roughly fit the binding sites. Any of the programs may be used alone or in combination.

If necessary, the three-dimensional structure may be “cleaned up” by modifying the atom types of the ligand, if present, and any water molecules that are present so that the water molecules find their lowest energy rotamer (i.e., are rotated to provide optimal interactions with the protein). The software may also be used to add hydrogens in standardized geometry (i.e., most favorable protonation state) with optimization of orientations of OH, SH, NH₃⁺, Met methyls, Asn and Gin sidechain amides, and His rings. Suitable software for performing this “clean up” includes, but is not limited to, SYBYL™ (Tripos, Inc.), WHATCHECK (part of CCP4 suite, COLLABORATIVE COMPUTATIONAL PROJECT, No. 4, Acta Cryst. D50: 760-763 (1994)), REDUCE (Word et al., J. Mol. Bio. 285: 1733-45 (1999)), and other functionally related computer programs. Any suitable docking computer program may be used to further validate the refined peptide crystal structure. Any of the programs may be used alone or in combination.

Three-Dimensional Structure of VEGFR2KD

Analogous to previously reported structures of both serine/threonine and tyrosine protein kinases, it has been discovered that the liganded, phosphorylated VEGFR2KD of the invention is folded into two domains with catalysis of phosphotransfer taking place in a cleft between the two lobes (See, e.g., the reported kinase structures found in Knighton, D. R. et al, Science 253: 407-413 (1991); Johnson, L. N. et al, Cell 85: 149-158 (1996); Hubbard, S. R. et al., Nature 372: 746-754 (1994)). The N-terminal lobe (approximately residues 810-920 of SEQ ID NO: 2) folds into a twisted beta sheet with one α elix (αC). The larger C-terminal domain (approximately residues 921-1168 of SEQ ID NO: 2) contains two beta strands, which lie at the top of the C-terminal domain adjacent to the N-terminal beta-sheet, and seven a helices. Like other protein kinases, VEGFR2KD contains functionally important loop regions: the glycine-rich nucleotide binding loop (approximately residues 840-848 of SEQ ID NO: 2, the catalytic loop (approximately residues 1026-1033 of SEQ ID NO: 2), and the activation loop (approximately residues 1046-1075 of SEQ ID NO: 2).

Through analysis of the crystalline structures of VEGFR2KD in complex with structurally diverse Compounds 3, 4, and 5, it has been discovered that the VEGFR2KD:ligand complexes of the invention comprise a ligand binding pocket that differs substantially from the unliganded, phosphorylated VEGFR2KD structure previously reported. Particularly, the structure of the VEGFR2KD:ligand complexes defines a unique ligand binding pocket of approximate dimensions 12 Å×9 Å×25 Å. Depictions of the VEGFR2KD ligand-binding pocket are shown in FIGS. 1, 2A, and 2B. The ligand binding pocket lies in the cleft between the N-terminal and C-terminal domains of the kinase of SEQ ID NO: 2. Using the program Insight II version 98.0 (Molecular Simulations Incorporated, San Diego, Calif.), it has been discovered that VEGFR2KD comprises a ligand binding pocket that is defined by the structural coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2 or a conservatively substituted variant thereof.

A binding pocket defined by the atomic coordinates of the amino acids, as set forth in Table 1, Table 2, Table 3, Table 4, and/or Table 5, or a binding pocket whose root mean square deviation from the atomic coordinates of the backbone atoms of these amino acids that is not more than about 0.90 Å, is a binding pocket of a VEGFRKD or of a protein structurally related thereto. Preferably the root mean square deviation is not more than about 0.75 Å. More preferably, the root mean square deviation is not more than about 0.90 Å.

It has also been discovered that the kinase activation loop of the VEGFR2KD:ligand complexes of the invention is folded in a unique conformation that creates a deep crevice and which makes specific packing and electrostatic interactions with ligands (e.g. inhibitors). As shown in FIG. 1, the loop starts after beta strand 8 in the cleft between the two domains and follows a path that first extends (residues 1046 through 1050 of SEQ ID NO: 2) towards the N-terminal domain, then turns (residues 1050 through 1053 of SEQ ID NO: 2) towards the C-terminal domain, forms another turn (residues 1053 through 1059 of SEQ ID NO: 2), extends in a short B-strand out towards solvent (residues 1059 through 1062 of SEQ ID NO: 2), makes a turn (residues 1062 through 1065 of SEQ ID NO: 2), forms another short B-strand (residues 1065 through 1068 of SEQ ID NO: 2) that goes back in towards the protein C-terminal domain, and forms a short loop (residues 1069 through 1075 of SEQ ID NO: 2) that connects with α helix EF.

For protein kinases in general, in the activated (usually phosphorylated) form, this loop adopts an “open” conformation that permits the catalytically competent binding of Mg-ATP and substrates. In the non-activated form, generally non-phosphorylated, many different conformations of this loop have been reported for various kinases (reviewed in Johnson, L. N. et al., Cell 85: 149-158 (1996)). The conformation observed in the structures of unphosphorylated VEGFR2KD bound to ligands is unique to those already described. In the published structure of the unliganded, phosphorylated VEGFR2KD a conformation for most of the activation loop (residues 1048-1063 SEQ ID NO: 2) could not be modeled due to a lack of interpretable electron density, most likely caused by dynamic disorder. By comparison, in the unphosphorylated VEGFRKD:ligand complexes of the invention, amino acid residues 1046-1060 of SEQ ID NO: 2 are well-ordered and adopt a conformation that is unique to those published for other protein kinases.

In the VEGFR2KD structures, the sidechains of Cys1045 and Phe1047 point in towards the ligand binding cavity and the shape and electronic features of these amino acid residues primarily determine this portion of the binding site. The phenyl ring of Phe1047 makes aromatic and hydrophobic interactions with ligands bound at this site, and a large sulfhydryl of Cys1045 occupies space not occupied in known structures. These differences substantially affect the shape and chemical nature of this portion of the ligand binding site.

It will be readily apparent to those of skill in the art that the numbering of amino acids in structurally related forms of VEGFRKD may be different than that set forth herein. Corresponding amino acids in other forms are readily identified by inspection of the amino acid sequences, for example, through the use of commercially available homology software programs as described above.

The amino acids of the VEGFRKD, and subsets thereof, including the ligand binding pocket, of the invention are described herein in reference to the set of structure/atomic coordinates set forth in Tables 1, 2, 3, 4 and/or 5. The terms “structure coordinates” (or structural coordinates) and “atomic coordinates” refer to Cartesian coordinates derived from mathematical equations related to the patterns obtained on diffraction of a monochromatic beam of X-rays by the atoms (scattering centers) of a protein or protein-ligand complex in crystal form. The diffraction data are used to calculate an electron density map of the repeating unit of the crystal. The electron density maps are then used to establish the positions of the individual atoms of the enzyme or enzyme complex.

The variations in coordinates discussed above may be generated because of mathematical manipulations of the VEGFRKD crystal complex structure coordinates. For example, the structure coordinates set forth in Table 1, Table 2, Table 3, Table 4, or Table 5 may be manipulated by crystallographic permutations of the structure coordinates, fractionalization of the structure coordinates, integer additions, subtractions to sets of the structure coordinates, coordinate transformations, e.g., translation or rotation, or combinations thereof.

Alternatively, modifications in the crystal structure due to mutations, additions, substitutions, and/or deletions of amino acids, or other changes in any of the components that make up the crystal may also account for variations in structure coordinates. If such variations are within an acceptable standard error as compared to the original coordinates, the resulting three-dimensional shape is considered to be the same. Thus, for example, a ligand that binds to a VEGFR2KD ligand binding pocket would also be expected to bind to a binding pocket whose atomic coordinates, when compared to those described herein, have a RMS deviation of not more than about 0.90 Å, preferably not more than about 0.90 Å, more preferably not more than about 0.75 Å, and most preferably not more than about 0.60 Å, from the backbone atoms.

Various computational analyses can be performed to determine whether a polypeptide or the binding pocket portion thereof is sufficiently similar to the VEGFR binding pocket as described herein. Such analyses may be carried out through the use of known software applications, such as the MODELLER module of INSIGHT II (Accelrys, Inc., San Diego, Calif.), ProMod (University of Geneva, Switzerland), SWISS-MODEL (Swiss Institute of Bioinformatics), and the Molecular Similarity application of QUANTA (Accelrys, Inc., San Diego, Calif.).

Programs such as QUANTA (Accelrys, Inc., San Diego, Calif.), INSIGHT II (Accelrys, Inc., San Diego, Calif.), Maestro (Schrödinger, Portland, Oreg.), SYBYL (Tripos, Inc., St. Louis, Mo.), and MacroModel (Schrödinger, Portland, Oreg.) permit comparisons between different structures, different conformations of the same structure, and different parts of the same structure. Comparison of structures using such computer software may involve the following steps: 1) loading the structures to be compared; 2) defining the atom equivalencies in the structures; 3) performing a fitting operation; and 4) analyzing the results.

In comparing structures, each structure is identified by a name. One structure is identified as the target (i.e., the fixed structure); all remaining structures are working structures (i.e., moving structures). Since atom equivalency with QUANTA is defined by user input, as defined herein “equivalent atoms” refers to protein backbone atoms (N, Cα, C, and O) for all conserved residues between the two structures being compared.

When a rigid-fitting method is used, the working structure is translated and rotated to obtain an optimum fit with the target structure. The fitting operation uses an algorithm that computes the optimum translation and rotation to be applied to the moving structure, such that the root-mean-square difference of the fit over the specified pairs of equivalent atoms is an absolute minimum. This number, given in angstroms (Å), is reported by software applications such as QUANTA (Accelrys, Inc., San Diego, Calif.) or other similar programs. Any molecule or molecular complex or binding pocket thereof that has a root-mean-square deviation of conserved residue backbone atoms (N, Cα, C, O) of less than about 0.5 Å when superimposed on the relevant backbone atoms described by structure coordinates listed in any one of Tables 1-5 are considered identical.

The atomic coordinates and thus, three-dimensional structure, may also be used in homology modeling or NMR spectroscopy for drug design, for example, as described below.

Computers and Computer Software

Once the atomic coordinates are known, a computer may be used for producing a three-dimensional representation of the VEGFRKD, a structurally related peptide, or a VEGFRKD or VEGFRKD-like ligand binding pocket. Thus, the invention relates to a method for generating a three-dimensional computer representation of a molecule comprising VEGFRKD, or a peptide that is structurally related thereto, comprising applying the atomic coordinates set forth in Table 1, Table 2, Table 3, Table 4, or Table 5, or a related set of atomic coordinates having a root mean square deviation of not more than about 1.25 Å away from the core Cα atoms of the atomic coordinates as set forth in Table 1, Table 2, Table 3, Table 4, or Table 5, to a computer algorithm to generate a three-dimensional representation of the molecule.

Similarly, the invention includes a method for generating a three-dimensional computer representation of a VEGFRKD or VEGFRKD-like ligand binding pocket comprising applying the atomic coordinates set forth in Table 1, Table 2, Table 3, Table 4, or Table 5, or a related set of atomic coordinates having a root mean square deviation of not more than about 0.90 Å away from the core Cα atoms of the atomic coordinates as set forth in Table 1, Table 2, Table 3, Table 4, or Table 5, to a computer algorithm to generate a three-dimensional representation of the binding pocket. In one embodiment, the binding is defined by the structural coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2 or a conservatively substituted variant thereof.

This is achieved through the use of commercially available software that is capable of generating three-dimensional graphical representations of molecules or portions thereof from a set of atomic coordinates.

Suitable computers are known in the art and typically include a central processing unit (CPU) and a working memory, which can be random-access memory, core memory, mass-storage memory, or a combination thereof. The CPU may encode one or more programs. Computers also typically include display, input and output devices, such as one or more cathode-ray tube display terminals, keyboards, modems, input lines and output lines. Further, computers may be networked to computer servers (the machine on which large calculations can be run in batch) and file servers (the main machine for all the centralized databases).

Machine-readable media containing data, such as the crystal atomic coordinates of the polypeptides, may be inputted using various hardware, including, but not limited to, modems, CD-ROM drives, disk drives, or keyboards.

Machine-readable data medium can be, for example, a floppy diskette, hard disk, or an optically-readable data storage medium, which can be either read only memory, or rewritable, such as a magneto-optical disk.

Output hardware, such as a CRT display terminal, may be used for displaying a graphical representation of the ligand-binding pocket of the VEGFRKD polypeptides described herein. Output hardware may also include a printer and disk drives.

The CPU coordinates the use of the various input and output devices, coordinates data access from storage and access to and from working memory, and determines the sequence of data processing steps. A number of programs may be used to process the machine-readable data. Such programs are discussed herein in reference to the computational methods of drug discovery.

Drug Design and Computer Modeling

Once the three-dimensional structure of a crystal comprising a unphosphorylated VEGFRKD:ligand complex is determined, a chemical entity (e.g. a potential inhibitor or enhancer), may be computationally evaluated for its ability to associate with VEGFRKD, a structurally related peptide, or a VEGFRKD or VEGFRKD-like binding pocket. The chemical entity may be computationally evaluated using a docking program, such as FelxiDock (Tripos, St. Louis, Mo.), GRAM (Medical Univ. of South Carolina), DOCK (Univ. of California at San Francisco), Glide (Schrödinger, Portland, Oreg.), Gold (Cambridge Crystallographic Data Centre, UK), FlexX (BioSolvelT GmbH, Germany), AGDOCK (Gehlhaar et al., Chemistry & Biol. 2:317-324 (1995); Bouzida et al., Pacific Symp. on Biocomputing 99: 426-437 (1999); Bouzida et al., Internat. J of Quantum Chem. 72:73-84 (1999); Gehlhaar et al., Proceedings of the Seventh Ann. Conf on Evolutionary Programming, The MIT Press, Cambridge, Mass. (1998)), Hex (Ritchie et al., Proteins: Struct. Funct. & Genet. 39:178-194 (2000)), or AUTODOCK (Scripps Research Institute, La Jolla, Calif.). The modeling procedure can include computer fitting of potential ligands to the VEGFRKD or VEGFRKD-like ligand-binding pocket to ascertain how well the shape and the chemical structure of the potential ligand will complement or interfere with the ligand-binding pocket (Bugg et al., Scientific American Dec.:92-98 (1993); West et al., TIPS 16:67-74 (1995)).

Compounds that bind to a VEGFRKD or VEGFRKD-like ligand-binding pocket may also be designed. The design process involves the consideration of at least two factors. First, the entity must be capable of physically and structurally associating with some of or the entire ligand-binding pocket. The phrase “associating with” refers to a condition of proximity between a chemical entity and a binding pocket on a protein. The association may be non-covalent, for example, wherein the juxtaposition is energetically favored by hydrogen bonding of van der Waals or electrostatic interactions, or it may be covalent. Non-covalent molecular interactions contributing to this association include, but are not limited to, hydrogen bonding, van der Waals interactions, hydrophobic interactions, and electrostatic interactions.

Second, the entity must be able to assume a conformation that allows it to associate with the VEGFRKD or VEGFRKD-like ligand-binding pocket directly. Although certain portions of the entity will not directly participate in these associations, those portions of the entity may still influence the overall conformation of the molecule. This, in turn, may have a significant impact on potency. Conformational requirements include, but are not limited to, the overall three-dimensional structure and orientation of the chemical entity in relation to all or a portion of the binding pocket, and the spacing between functional groups of an entity comprising several chemical entities that directly interact with the ligand binding pocket.

The potential modulatory effect or binding ability of a chemical entity on a VEGFRKD or VEGFRKD-like ligand-binding pocket may be analyzed prior to its actual synthesis and testing through the use of computer-modeling techniques. If, from the theoretical structure of the given entity, it can be surmised that there is insufficient interaction and association between it and the ligand binding pocket, further testing of the entity may not be prudent. However, if computer modeling indicates a strong interaction, then the molecule can be synthesized and tested for its ability to bind to a VEGFRKD or VEGFRKD-like ligand binding pocket. This may be achieved by testing the ability of the molecule to modulate VEGFRKD activity using the assays described in U.S. Pat. No. 6,316,603. Using this scheme, the fruitless synthesis of compounds with poor binding activities may be avoided.

A potential inhibitor of a VEGFR, or structurally related peptide, may be computationally evaluated by means of a series of steps in which chemical entities are screened and selected for their ability to associate with a VEGFRKD or VEGFRKD-like ligand binding pocket. One skilled in the art may use one of several methods to screen chemical entities or fragments for their ability to associate with a VEGFRKD or VEGFRKD-like ligand-binding pocket. For example, the artisan may visually inspect a VEGFRKD or VEGFRKD-like ligand-binding pocket on a computer screen based on the atomic coordinates reported in Table 1, Table 2, Table 3, Table 4, or Table 5, or a portion thereof, or coordinates that define a similar shape generated from the machine-readable storage medium. Selected chemical entities may then be positioned in a variety of orientations, or docked, within that binding pocket as described herein. Docking may be accomplished using software, such as Quanta (Accelrys, Inc., San Diego, Calif.) or SYBYL (Tripos, Inc., St. Louis, Mo.), followed by energy minimization and molecular dynamics with standard molecular mechanics force fields, such as CHARMM (Department of Chemistry & Chemical Biology, Harvard Univ., Cambridge, Mass.) or AMBER (School of Pharmacy, Department of Pharmaceutical Chemistry, University of California at San Francisco, Calif.).

Specialized computer programs to assist in the process of selecting chemical entities include, but are not limited to, those described in the following references, which are incorporated by reference herein:
1. GRID (Goodford, J. Med. Chem. 28:849-857 (1985)). GRID is available from the Oxford University, Oxford, UK.
2. MCSS (Miranker et al., Proteins: Struct. Funct. and Genet 11:29-34 (1991)). MCSS is available from Accelrys, Inc., San Diego, Calif.
3. AUTODOCK (Goodsell et al., Proteins: Struct Funct. and Genet. 8:195-20 (1990)). AUTODOCK is available from the Scripps Research Institute, La Jolla, Calif.
4. DOCK (Kuntz et al., J. Mol. Biol. 161:269-288 (1982)). DOCK is available from the University of California, San Francisco, Calif.
5. GOLD (Jones et al., J. Mol. Biol 267:727-748 (1997)). GOLD is available from the Cambridge Crystallographic Data Centre, UK.
6. GLIDE (Eldridge et al., J. Comput. Aided Mol. Des. 11:425-445 (1997)). Glide is available from Schrödinger, Portland Oreg.

Once suitable chemical entities have been selected, they can be assembled into a single compound or complex. Assembly may be preceded by visual inspection of the relationship of the fragments to each other on the three-dimensional image displayed on a computer screen in relation to the atomic coordinates set forth in Table 1, Table 2, Table 3, Table 4, or Table 5, or a portion thereof. This can be followed by manual model building using software such as QUANTA or SYBYL. Useful programs to aid one of skill in the art in connecting the individual chemical entities include, but are not limited to, those described in the following references, which are incorporated by reference herein:
1. CAVEAT (Bartlett et al., Molecular Recognition in Chemical and Biological Problems, Special Pub., Royal Chem. Soc., 78, pp. 182-196 (1989); Lauri et al., J. Comput. Aided Mol. Des. 8:51-66 (1994)). CAVEAT is available from the University of California, Berkeley, Calif.
2. ISIS: See Martin, J. Med. Chem. 35:2145-2154 (1992)). ISIS is available from MDL Information Systems, San Leandro, Calif.
3. HOOK (Eisen et al., Proteins: Struct, Funct., Genet. 19:199-221 (1994)). HOOK is available from Accelrys, Inc., San Diego, Calif.

Instead of proceeding to build an inhibitor of a VEGFR, or a structurally related peptide, in a step-wise fashion one chemical entity at a time, as described above, modulatory compounds may be designed as a whole or de novo using either an empty binding site or, optionally, including some portion(s) of a known modulator. There are many known de novo ligand design methods, such as LeapFrog (available from Tripos Associates, St. Louis, Mo.) and those discussed in the following references, which are incorporated by reference herein.
1. LUDI (Bohm, J. Comp. Aid. Molec. Design. 6:61-78 (1992)). LUDI is available from Accelrys Inc., San Diego, Calif.
2. SPROUT (Gillet et al., J. Comput. Aided Mol. Design. 7:127-153 (1993)). SPROUT is available from the University of Leeds, UK.

Other molecular modeling techniques may also be employed, such as those described in Cohen et al, J. Med. Chem. 33:883-894 (1990); Navia et al., Curr. Opin. Struct. Biol. 2:202-210 (1992); Balbes et al., Reviews in Computational Chemistry, Vol. 5, K. Lipkowitz et al., eds., VCH, New York, N.Y., pp. 337-380 (1994); or Guida, Curr. Opin. Struct. Biol. 4:777-781 (1994).

Once a chemical entity has been designed or selected by using such methods, or functionally similar methods, the efficiency with which that entity binds to a VEGFRKD or VEGFRKD-like ligand-binding pocket may be tested and optimized by computational evaluation. For example, an effective VEGFR modulator preferably demonstrates a relatively small difference in energy between its bound and free states (i.e., a small deformation energy of binding). VEGFR modulators may interact with the KD ligand-binding pocket in more than one conformation that is similar in overall binding energy. In those cases, the deformation energy of binding is taken to be the difference between the energy of the free entity and the average energy of the conformations observed when the inhibitor binds to the protein.

An entity designed or selected as binding to a VEGFRKD or VEGFRKD-like ligand-binding pocket may be further computationally optimized so that, in its bound state, it would preferably lack repulsive electrostatic interaction with the target kinase and with the surrounding water molecules. Such non-complementary electrostatic interactions include, but are not limited to, repulsive charge-charge, dipole-dipole and charge-dipole interactions.

Suitable computer software is available to evaluate compound deformation energy and electrostatic interactions. Examples of programs designed for such uses include, but are not limited to, Gaussian (Frisch, Gaussian, Inc., Carnegie, Pa.), AMBER (Kollman, University of California at San Francisco), Jaguar (Schrödinger, Portland, Oreg.); SPARTAN (Wavefunction, Inc., Irvine, Calif.), QUANTA/CHARMM (Accelrys, Inc., San Diego, Calif.), Impact (Schrödinger, Portland, Oreg.), Insight II/Discover (Accelrys, Inc., San Diego, Calif.), MacroModel (Schrödinger, Portland, Oreg.), Maestro (Schrödinger, Portland, Oreg.), DelPhi (Accelrys, Inc., San Diego, Calif.), and AMSOL (Quantum Chemistry Program Exchange, Indiana University). These programs may be implemented, for instance, using workstations produced by companies, such as Silicone Graphics, Hewlet Packard, Sun Microsystems, and International Business Machines.

In another approach, small-molecule databases are computationally screened to determine their potential to bind in whole, or in part, to a VEGFRKD or VEGFRKD-like ligand-binding pocket. Computer programs can be employed to estimate the attraction, repulsion, and steric hindrance of the ligand to the binding pocket. For example, one can screen computationally small molecule databases for chemical entities or compounds that can bind in whole, or in part, to VEGFRKD. In this screening, the quality of fit of such entities or compounds to the binding site may be judged either by shape complementarity or by estimated interaction energy (Meng et al., J. Comp. Chem., 13:505-524 (1992)). Generally, the tighter the fit (e.g., the lower the steric hindrance and/or the greater the attractive force), the more potent the drug is projected to be since these properties are consistent with a tighter-binding constant.

Initially, a potential ligand can be obtained by screening a random chemical library. A ligand selected in this manner could be then be systematically modified by computer-modeling programs until one or more promising potential ligands are identified. Such analysis has been shown to be useful in the design of, for example, HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)). Such computer modeling allows the selection of a finite number of rational chemical modifications, as opposed to the potentially unlimited number of essentially random chemical modifications that could be made, any one of which might lead to a drug. Each chemical modification requires additional chemical steps, which, while being reasonable for the synthesis of a finite number of compounds, quickly becomes overwhelming if all possible modifications needed to be synthesized. Thus, through the use of the atomic coordinates disclosed herein and computer modeling, a large number of these compounds can be rapidly modeled via a computer, and a few promising candidates can be determined without the laborious synthesis of a multitude of compounds.

Once a potential ligand (agonist or antagonist) is identified, it can be either selected from commercial libraries of compounds or, alternatively, the potential ligand may be synthesized de novo. The prospective drug can be tested in inhibitor assays such as those described, for example, in U.S. Pat. No. 6,316,603 and Example 6 to test its ability to bind to the VEGFRKD or VEGFRKD-like ligand-binding pocket, and to modulate VEGFR kinase activity.

When a suitable compound is identified, supplemental crystals may be grown comprising a protein-ligand complex of a VEGFR and a ligand or a VEGFRKD and a ligand. Preferably, the crystals effectively diffract X-rays allowing for the determination of the atomic coordinates of the protein-ligand complex to a resolution of greater than or equal to about 3.0 Å, more preferably, greater than or equal to about 2.0 Å. Molecular Replacement, described below, or a functionally similar technique, can be used to determine the three-dimensional structure of the supplemental crystals using the atomic coordinates set forth in Table 1, Table 2, Table 3, Table 4, and/or Table 5.

Thus, the structure coordinates set forth in Table 1, Table 2, Table 3, Table 4, and/or Table 5 can also be used to obtain structural information about another crystallized molecule or molecular complex. This may be achieved by any suitable known technique, such as molecular replacement. By using molecular replacement, all or part of the structure coordinates of the VEGFR crystal complexes of the invention can be used to determine the structure of a crystallized molecule or molecular complex whose structure is unknown. This process is more efficient than attempting to determine such information ab initio.

Molecular replacement provides an accurate estimation of the phases for an unknown structure. Phases constitute a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure can provide a an estimate of the phases for the unknown structure.

Molecular replacement involves generating a preliminary model of a molecule or molecular complex whose structure coordinates are unknown, by orienting and positioning the relevant portion of the VEGFR crystal complex according to any one of Tables 1-5 within the unit cell of the crystal of the unknown molecule or molecular complex so as best to theoretically account for the observed X-ray diffraction data of the crystal of the molecule or molecular complex whose structure is unknown. Phases can then be calculated from this model and combined with the observed X-ray diffraction data amplitudes to generate an electron density map of the structure whose coordinates are unknown. This, in turn, can be subjected to any known model building and structure refinement techniques to provide a final, accurate structure of the unknown crystallized molecule or molecular complex (See, e.g., Lattman, Meth. Enzymol. 115:55-77 (1985); Rossmann, ed., “The Molecular Replacement Method,” Int. Sci. Rev. Ser., No. 13, Gordon & Breach, New York (1972)). Thus, the structure of any portion of any crystallized molecule or molecular complex that is sufficiently homologous to any portion of the VEGFR complex can be resolved by this method.

Computer programs that can be used for this purpose include, but are not limited to, those described in the following references, which are incorporated by reference herein:
1. X-PLOR (Brunger, “X-PLOR:A System for X-ray Crystallography and NMR,” Yale University Press, New Haven, Conn. (1992)). X-PLOR is commercially available from Accelrys, Inc., San Diego, Calif.;
2. EPMR (Kissinger et al., Acta Cryst. D55:484-491 (1999));
3. AMORE (Navaza, J., Acta Crystallographics ASO, 157-163 (1994)). AMORE is commercially available from Collaborative Computing Project #4 (CCP4), Danesbury Laboratory, Warrington, UK;
4. QUANTA, which is commercially available from Accelrys, Inc., San Diego, Calif.;
5. INSIGHT, which is commercially available from Accelrys, Inc., San Diego, Calif.;
6. ARP/wARP (Perrakis et al., Nature Struc. Biol. 6:458-463 (1999); Lamzin et al., Acta Cryst. D49:129-147 (1993)). ARP/wARP is commercially available from the European Molecular Biology Laboratory, Heidelberg, Germany; and
7. ICM, which is commercially available from MolSoft, La Jolla, Calif.

In another preferred embodiment, a method of molecular replacement is utilized to obtain structural information about a VEGFR other than VEGFR2. The structure coordinates of VEGFR2 crystal complexes as described herein are useful in solving the structure of other isoforms of VEGFR or other VEGFR containing complexes.

Furthermore, the structure coordinates of the VEGFR polypeptides, described herein, are useful in solving the structure of other VEGFR proteins that have amino acid substitutions, additions and/or deletions. These VEGFR mutants may optionally be crystallized in complex with a chemical entity, such as one of the ligand listed above. The crystal structure of such a complex may then be solved by molecular replacement and compared with structure of the VEGFR polypeptides described herein. Potential sites for modification within the various binding sites of the enzyme may thus be identified. This information provides an additional tool for determining the efficient binding interactions, for example, increased hydrophobic interactions, between VEGFR and a chemical entity.

The structure coordinates are also useful to solve the structure of crystals of VEGFR homologues complexed with chemical entities. This approach enables the determination of the important sites for interaction between chemical entities, including potential VEGFR modulators with the VEGFR ligand binding site. For example, high resolution X-ray diffraction data collected from crystals exposed to different types of solvent allows the determination of where each type of solvent molecule resides. Small molecules that bind tightly to those sites can then be designed and synthesized and tested for their ability to modulate VEGFR activity.

All of the complexes referred to above may be studied using known X-ray diffraction techniques and may be refined versus about 1.0 to about 3.0 Å resolution X-ray data to an R value of about 0.20 or less using computer software, such as X-PLOR (Brunger, 1992, supra, distributed by Accelrys, Inc., San Diego, Calif. This information may be used to optimize known VEGFR modulators, and to design new VEGFR modulators.

For all of the drug design strategies described herein, successive iterations of any and/or all of the steps provided by the aforementioned procedures are typically performed to yield one or more ligands with improved properties (e.g., activity).

The following examples are for the purpose of illustrating various embodiments and features of the invention.

### EXAMPLES

### Example 1

### Purification and Biochemical Analysis of VEGFR2KD Polypeptides Cloning a VEGFR2 Protein

The coding sequence (Terman et al., Biochem Biophys. Res. Commun. 187:1579-86 (1992)) for the cytoplasmic domain of the VEGFR2 was amplified by PCR (Mullis et al., Biotechnology 24:17-27 (1992)) from a human aorta cDNA pool (Clontech, Palo Alto, Calif.). Two overlapping sequences were amplified independently: Vcyt (amino acid residues M806-V1356), which represent the entire cytoplasmic domain, and Vcat (amino acid residues C817M-G1191), with boundaries based upon a primary amino acid sequence alignment with the insulin receptor kinase catalytic domain (Wei et al., J. Biol. Chem. 270: 8122-8130 (1995)).

The PCR oligonucleotide primer sequences for Vcyt were:

(SEQ ID NO: 6)

| | |
|---|---|
| Vcyt5 | 5′-CAGCATATGGATCCAGATGAACTCCCATTGG-3′ |
| and | |

(SEQ ID NO: 7)

| | |
|---|---|
| Vcyt3 | 5′-GCGGTCGACTTAAACAGGAGGAGAGCTCAGTGTG-3′. |

The PCR oligonucleotide primer sequences for Vcat were:

| | | |
|---|---|---|
| Vcat5 | 5′-GCACATATGGAACGACTGCCTTATGATGCCAGC-3′ | (SEQ ID NO: 8) |
| and | | |
| Vcat3 | 5′-CCTGTCGACTTATCCAGAATCCTCTTCCATGCTCAAAG-5′. | (SEQ ID NO: 9) |

The amplified DNA was digested with the restriction enzymes NdeI and SalI, ligated into the E. coli plasmid pET24a (Novagen, Madison, Wis.) and sequence verified (SEQ ID NO: 1). When compared to the original VEGFR2 sequence in Genbank (Accession number 346345), two nucleotide differences were noted that resulted in codon changes (Glu848Val and Asn835Lys) in both Vcyt and Vcat. Our sequence (SEQ ID NO: 1) agrees with subsequent VEGFR2 Genbank submissions (Accession numbers 2655412 and 3132833).

Mutations were introduced by oligonucleotide site directed mutagenesis (Kunkel, T. A., Proc. Natl. Acad. Sci. USA, 82: 488-492 (1985)) using the Muta-Gene in vitro Mutagenesis Kit (Bio-Rad, Hercules, Calif.). The Vcat DNA fragment was subcloned from the pET24a vector using an NdeI-XhoI digest into the vector pMGH4 (Schoner et al., Proc. Natl. Acad. Sci. USA, 83: 8506-8510 (1986); Kan et al., J. Protein Chem., 11: 467-73 (1992)) and this vector was used to generate the ssDNA uracil template (minus strand) in E. coli strain CJ236 supplied in the kit. An oligo

(SEQ ID NO: 10)

5′-CTCAGCAGGATTGATAAGACTACATTGTTC-3′

was designed to create a construct (Vcat(Δ G1172-G 1191)) which truncated the C-terminus to amino acid residue D 1171. Another oligo

(SEQ ID NO: 11)

5′-GAATTTGTCCCCTACAAGGAAGCTCCTGAAGATCTG-3′

was designed to delete the central 50 amino acid residues (amino acid residues T940-E989) of the insert kinase domain, based on a sequence alignment with FGFR1 (Mohammadi et al., Cell 86: 577-87 (1996)). Sequence analysis detected an inadvertent Glu990-Val mutation. All DNA modification and restriction enzymes were purchased from New England Biolabs and oligonucleotides were purchased from Genosys Biotechnology.

The VEGFR2KD polypeptide (SEQ ID NO: 3) was made in several steps to combine the necessary mutations into the baculovirus expression vector pAcSG2 (Pharmingen San Diego, Calif.):
Step 1: the coding region for Vcyt was PCR subcloned from the pET24a vector into the NcoI-KpnI sites of vector pAcSG2.
Step 2: a 2358 bp ScaI-BglII DNA fragment from plasmid pMGH4-Vcat (Δ T940-E989, E990V) was ligated to a 1695 bp BglII-ScaI DNA fragment from pMGH4-Vcat (Δ G1172-G1191) creating a pMGH4-Vcat (Δ T940-E989, E990V, Δ G1172-G1191) vector.
Step 3: a 913 bp BstEII-Eagl DNA fragment a pMGH4-Vcat (Δ T940-E989, E990V, Δ G1172-G1191) was ligated to a 3290 bp Eagl-BstEII DNA fragment from pAcSG2-Vcyt creating pAcSG2-Vcyt (Δ T940-E989, E990V, Δ G1172-G1191), also referred to as VEGFR2KD (SEQ ID NO: 3). This final construct was sequence verified through the entire coding region and confirmed to contain only these known mutations from the wild-type sequence.

DNA encoding VEGFR2KD was transfected into Sf9 cells with linearized baculovirus DNA according to the protocol of the manufacturer (Pharmingen, San Diego, Calif.). Single plaques were isolated from this transfection and high titer stocks generated. All stocks were examined by isolation of baculoviral DNA and PCR amplification of the insert using the polyhedron forward and reverse primers (Invitrogen, San Diego, Calif.). Sf21 cells were infected at 1-1.5 million cells/mL at MOI=5 for 72 hours and harvested by centrifugation.

Purification of VEGFR2KD from Sf21 Cells

Cell pellets were lysed by dounce homogenization and sonication in 20 mM Tris (pH 8.0), 20 mM NaCl, 5 mM DTT, and 5% (v/v) glycerol. The lysate was centrifuged for 50 minutes at 35,000 rpm in a Ti45 rotor. The soluble fraction was loaded onto a 40 mL Q-30 anion exchange column (Pharmacia) and eluted with a 20 mM to 600 mM NaCl gradient in 20 mM Tris (pH 8.0), 5 mM DTT, and 5% (v/v) glycerol over 20 column volumes. VEGFR2KD protein was pooled by SDS-PAGE gel analysis and by the presence of kinase activity as measured against gastrin substrate peptide substrate (Boehringer Mannheim). Pooled material was loaded onto a 40 mL hydroxyapatite (Bio-Rad) column and washed extensively with 20 mM Tris (pH 8.0), 50 mM NaCl, 5 mM DTT, and 5% glycerol. Protein was eluted using a 500 mL linear gradient from 0 to 50 mM potassium phosphate (pH 8.0), 50 mM NaCl, 5 mM DTT, and 5% glycerol. VEGFR2KD (SEQ ID NO: 3) protein was pooled by SDS-PAGE gel analysis and by the presence of kinase activity as measured against the gastrin peptide. Material from this column was then diluted 1:1 with 20 mM Tris (pH 8.0), 20 mM NaCl, 5 mM DTT, and 5% glycerol and loaded onto an 8 mL Q-15 anion exchange column (Pharmacia). Protein was eluted using with a 180 mL linear NaCl gradient (20 mM-175 mM) in 20 mM Tris (pH 8.0), 5 mM DTT, and 5% glycerol. VEGFR2KD (SEQ. ID NO: 3) protein was pooled as described above. 4 M (NH₄)₂SO₄ was added to the pool to final concentration of 0.6 M and the pool loaded onto a 10 mL HP-phenyl Sepharose column (Pharmacia). VEGFR2KD (SEQ. ID NO: 3) protein was eluted using a 200 mL linear reverse gradient from 0.6 M to 0 M (NH₄)₂SO₄ in 20 mM Tris and 5 mM DTT.

Purified VEGFR2KD protein (SEQ ID NO: 3) was buffer exchanged into 50 mM HEPES (pH 7.5), 10 mM DTT, 10% glycerol, and 25 mM NaCl over a 500 mL G-25 column (Pharmacia) and concentrated to 1 mg protein/mL through a 10 kD cutoff polysulfone membrane (Amicon). Final material was aliquoted and flash frozen in liquid nitrogen and stored at −70° C.

The purified protein was then buffer exchanged over a Sephadex G-25 column (Pharmacia) into either: (1) 10 mM HEPES (pH 7.5), 50 mM NaCl, 10 mM DTT, and 5% (v/v) glycerol; or (2) 10 mM HEPES (pH 7.5), 10 mM NaCl, 10 mM DTT, and 5% (v/v) DMSO. The final material was concentrated through a 10 kD cutoff polysulfone membrane (Amicon), aliquoted, and flash frozen in liquid nitrogen and stored at −70° C.

### Example 2

### Crystallization and Structural Determination of VEGFR2KD:Compound 1 Complex

The conformation of VEGFR2KD described herein was first determined as a complex with Compound 1. Protein samples used for VEGFR2KD:Compound 1 complex crystallizations were prepared by thawing aliquots (on ice) of VEGFR2KD protein (SEQ. ID NO: 3) stored in 10 mM HEPES (pH 7.5), 50 mM NaCl, 10 mM DTT, and 5% (v/v) glycerol. The protein sample was then buffer exchanged into 10 mM HEPES (pH 7.5), 10 mM NaCl, and 10 mM DTT and concentrated. Initial crystallization conditions were found from a large random screen using the hanging drop vapor diffusion method. Each test crystallization drop was prepared by mixing approximately 2 μl of protein solution (VEGFR2KD at 3 mg/mL, 10 mM HEPES (pH 7.5), 10 mM NaCl, and 10 mM DTT) and an equal volume of the crystallization screening solution on a glass coverslip and suspending this coverslip above a reservoir of the same crystallization screening solution. This screen was conducted at 4° C. Preliminary crystals appeared using the Hampton Screen II #30 crystallization solution (Hampton Research Inc; solution 30 contains 100 mM HEPES (pH 7.5), 10% (w/v) polyethylene glycol (PEG) MW=6000, and 5% (v/v) 2,4-methylpentanediol (MPD)). Further refinement of these conditions, and microseeding with preliminary crystals, produced crystals large enough for X-ray diffraction data collection.

Microseeding was found to work best when the crystals from which the microseeds were gathered were less than 1 month old. The crystal used for data collection was grown at 13° C. by mixing 2 μl of protein solution (VEGFR2KD at 5 mg/mL, 100 mM HEPES (pH 7.5), 10 mM NaCl, and 10 mM DTT) with an equal volume of precipitant solution (100 mM HEPES (pH 7.5), 13% (w/v) PEG MW=6000 (Fluka Chemical Co.), 5% (v/v) MPD, and 200 mM ammonium sulfate) and suspending the drop above reservoir of the precipitant solution. After 12-18 hours the drop was microseeded by touching a rabbit whisker to a preliminary crystal of the VEGFR2KD:Compound 1 complex, then touching the rabbit whisker to the crystallization drop, and resealing the drop above the reservoir.

For X-ray data collection, a crystal of the VEGFR2KD:Compound 1 complex was transferred, using a fiber loop, to a cryoprotectant solution for approximately 3 seconds, flash frozen in liquid nitrogen, and placed in a stream of liquid nitrogen on the X-ray data collection apparatus. The cryoprotectant solution contained 100 mM HEPES (pH 7.5), 200 mM ammonium sulfate, 15% PEG MW=6000 (w/v), 30% (v/v) MPD, 500 μM AG-013121, and approximately 0.5% (v/v) DMSO.

An X-ray diffraction data set was collected using a Rigaku RU-200 rotating anode X-ray generator (CuKα radiation) and a MAR Research image plate detector. Data were integrated and scaled using the programs DENZO and SCALEPACK. Crystals of the VEGFR2KD:Compound 1 complex were found to belong the C2 space group containing one VEGFR2KD:Compound 1 complex per crystallographic asymmetric unit cell dimensions of a=136.25 Å, b=57.24 Å, c=52.10 Å, alpha=90°, beta=94.0°, and gamma=90°. The data set has an Rsym of 0.059 and contains 90.4% of the data in the resolution range of 20 to 2.2 Å.

Initial crystallographic phases for the VEGFR2KD:Compound 1 complex were found using the Molecular Replacement Analysis program EPMR (Kissinger et al., Acta Crystallographica D55: 484-91 (1999)) and the structure of unliganded phosphorylated VEGFR2KD as a starting model (U.S. Pat. No. 6,316,603; McTigue et al., Structure 7(3):319-30 (1999)). The atomic coordinates for the unliganded phosphorylated VEGFR2KD are publicly available through the Research Collaboratory for Structural Bioinformatics (RCSB) Protein Data Bank as entry 1VR2. The search model for molecular replacement contained protein atoms of amino acid residues 827 through 935, 1001 through 1045, and 1066 through 1168 of SEQ ID NO: 2 of the unliganded phosphorylated VEGFR2KD structure. The best molecular replacement solution was the top peak and had a correlation coefficient of 0.404 and an R-factor of 0.487 for data in the range of 15-4.0 Å. After Molecular Replacement Analysis, the model was subjected to rigid body, simulated annealing, and conjugant gradient refinement using the program Xplor (version 3.1) together with multiple rounds of manual fitting to electron density maps. The final model contains VEGFR2KD amino acid residues 822 through 938, 999 through 1060, and 1067 through 1168 of SEQ ID NO: 2, Compound 2, and 170 ordered water molecules. The R-factor for the final model is 0.217 for data with Fo>2σ in the resolution range 10-2.5 Å. The crystal coordinates of the VEGFR2KD:Compound 2 complex are provided in Table 1.

X-ray structures of other VEGFR2KD:ligand complexes, such as, for example, the ligands listed above, that crystallize in the same C2 crystal form with approximate cell dimensions of a=136 Å, b=57 Å, c=52 Å, alpha=90°, beta=94.0°, and gamma=90°, can be determined by difference Fourier and least-squares refinement methods using either the protein atoms of VEGFR2KD:Compound 1 complex structure, or another crystallographically isomorphous complex, as the initial structural model.

### Example 3

### Crystallization and Structural Determination of VEGFR2KD:Compound 2 Complex

Crystals of VEGFR2KD complexed with Compound 2 were grown by the following procedure. VEGFR2KD peptide (SEQ ID NO: 3) at a concentration of between 5 to 6 mg/mL in a storage buffer of 10 mM HEPES (pH 7.5), 10 mM NaCl, 10 mM dithiothreitol, and 5% (v/v) DMSO was thawed on ice from a storage temperature of −70° C. and the crystallization samples were subsequently kept at 4° C. throughout the entire crystallization process. Compound 2, as a 20 mM solution in DMSO, was added to the protein sample such that the final inhibitor concentration was approximately 500 μM. Exposure of the solutions containing Compoudn 2 to light was minimized to prevent photo-induced isomerization of Compound 2. Prior to setting up crystallization experiments, the sample was centrifuged at 13,000 rpm for approximately 6-8 minutes to remove any undissolved particulates.

Crystallization experiments were conducted according to the hanging drop vapor-diffusion method. 2 μl of the protein-inhibitor complex sample was placed on a plastic coverslip and mixed with 2 μl of a reservoir solution (100 mM HEPES (pH 7.5), 200 mM ammonium sulfate, 5% (v/v) MPD, and 15-20% (w/v) polyethylene glycol (MW=6000) (Fluka Chemical Co.)) and the coverslips were inverted and placed in sealed chambers above 0.5-1.0 mL of the reservoir solution. Before sealing the coverslips above the reservoirs, BME was added to the reservoirs to a final concentration of 60 mM, the coverslips were then sealed and placed in the dark to prevent photo-induced isomerization of Compound 2. After 12-18 hours of equilibration, the crystallization drops were microseeded by touching a rabbit whisker to a previously grown crystal of VEGFR2KD bound to Compound 2 and then touching the fiber to the drops containing VEGFR2KD and Compound 2. Small VEGFR2KD:Compound 2 complex crystals appeared in approximately one week. These small crystals were then macroseeded into fresh drops containing VEGFR2KD and Compound, prepared as above, which had equilibrated for 12-18 hours suspended above the crystallization reservoir solutions.

Macroseeding was done by dipping a crystal into the reservoir briefly to wash off any new seeds and then putting the washed crystal into an equilibrated drop. After approximately 1 week new crystals of approximate dimensions 0.1 mm×0.1 mm×0.5 mm appeared alongside the macroseed. The macroseed was never used for data collection as the macroseeds were always observed to have a higher degree of disorder than the crystals which grew alongside the macroseed.

For X-ray data collection, a crystal of the VEGFR2KD:Compound 2 complex was transferred, using a fiber loop, to a cryoprotectant solution for approximately 3 seconds, flash frozen in liquid nitrogen, and placed in a stream of liquid nitrogen on the X-ray data collection apparatus. The cryoprotectant solution contained 100 mM HEPES (pH 7.5), 200 mM ammonium sulfate, 15% PEG (w/v) (MW=6000), 30% (v/v) MPD, 500 μM AG-013736, and approximately, 2% (v/v) DMSO.

An X-ray diffraction data set was collected using a Rigaku RU-200 rotating anode X-ray generator (CuKα radiation) and a MAR300 MAR Research image plate detector. Data were integrated and scaled using the programs DENZO and SCALEPACK. Analysis of the data showed the crystals belong to space group C2 with unit cell dimensions of a=135.66 Å, b=57.22 Å, c=51.83 Å, alpha=90°, beta=93.441°, and gamma=90°. The agreement between multiply observed reflections (Rsym) is 0.051 for data in the range of 20-1.95 Å. Rsym is defined as (SUM (ABS(I−<I>))/SUM (I), where I represents the observed diffraction Intensity. The data set contains 99% of the unique reflections in the range of 20-1.95 Å.

The three-dimensional structure of the VEGFR2KD:Compound 2 complex was determined by standard refinement techniques using the structure of a VEGFR2KD:N-(4-piperazin-1-yl-3-trifluoromethyl-phenyl)-2-[3-((E)-styryl)-1H-indazol-6-ylsulfanyl]-benzamide complex as a starting model, which was derived through iterative cycles of structure solutions from the VEGFR2KD:Compound 1 complex structure (Example 2). Iterative cycles of least-squares refinement using Xplor (version 3.1) with manual fitting of the model to electron density maps resulted in a model with a final R-factor of 0.198 for data with Fo>2σ in the resolution range 8 to 1.95 Å. The final model contains atoms of VEGFR2KD, amino acid residues 816 through 938 and 998 through 1166 of SEQ ID NO: 2, Compound 2, and 281 water molecules. The crystal coordinates of the VEGFR2KD:Compound 2 complex are provided in Table 2.

### Example 4

### Identification of VEGFR2KD Binding Site

Amino acid residues were defined as forming part of the ligand binding site if they contain atoms whose positions are within about 5 Å of the position of a ligand atom. This analysis was calculated with InsightII (98.0) (Accelrys, San Diego, Calif.). This analysis was performed using the structural coordinates of VEGFR2KD in complex with Compounds 3, 4, and 5 (provided in Tables 3, 4, and 5, respectively), chosen for chemical diversity, and the residues defined as forming the ligand binding site are a composite of the residues that are within 5 Å of a ligand atom in these structures (See Table 6).

TABLE 1
Atomic Coordinates for
VEGFR2KD: 3-(1H-Pyrazolo[3,4-d]pyrimidin-4-ylsulfanylmethyl)-N-(3,4,5-
trimethoxy-phenyl)-benzamide (Compound 1)
Complex Crystalline Structure

| | | | | | | | | | |
|---|---|---|---|---|---|---|---|---|---|
| ATOM | 1 | CB | TYR | 822 | 40.630 | 20.424 | 40.608 | 1.00 | 25.50 |
| ATOM | 2 | CG | TYR | 822 | 39.987 | 21.220 | 41.750 | 1.00 | 28.06 |
| ATOM | 3 | CD1 | TYR | 822 | 40.399 | 21.114 | 43.131 | 1.00 | 27.04 |
| ATOM | 4 | CE1 | TYR | 822 | 39.810 | 21.904 | 44.153 | 1.00 | 21.22 |
| ATOM | 5 | CD2 | TYR | 822 | 38.964 | 22.139 | 41.460 | 1.00 | 24.80 |
| ATOM | 6 | CE2 | TYR | 822 | 38.392 | 22.909 | 42.483 | 1.00 | 19.23 |
| ATOM | 7 | CZ | TYR | 822 | 38.815 | 22.784 | 43.776 | 1.00 | 16.01 |
| ATOM | 8 | OH | TYR | 822 | 38.191 | 23.559 | 44.668 | 1.00 | 17.40 |
| ATOM | 9 | C | TYR | 822 | 42.239 | 22.212 | 40.432 | 1.00 | 28.87 |
| ATOM | 10 | O | TYR | 822 | 43.120 | 21.689 | 41.092 | 1.00 | 25.40 |
| ATOM | 11 | N | TYR | 822 | 42.352 | 20.610 | 38.612 | 1.00 | 29.39 |
| ATOM | 12 | CA | TYR | 822 | 41.429 | 21.316 | 39.561 | 1.00 | 28.27 |
| ATOM | 13 | N | ASP | 823 | 41.920 | 23.509 | 40.527 | 1.00 | 31.19 |
| ATOM | 14 | CA | ASP | 823 | 42.736 | 24.411 | 41.380 | 1.00 | 28.96 |
| ATOM | 15 | CB | ASP | 823 | 43.342 | 25.428 | 40.446 | 1.00 | 33.00 |
| ATOM | 16 | CG | ASP | 823 | 44.074 | 26.575 | 41.082 | 1.00 | 36.82 |
| ATOM | 17 | OD1 | ASP | 823 | 44.126 | 26.729 | 42.309 | 1.00 | 38.66 |
| ATOM | 18 | OD2 | ASP | 823 | 44.599 | 27.343 | 40.277 | 1.00 | 41.21 |
| ATOM | 19 | C | ASP | 823 | 42.057 | 25.056 | 42.563 | 1.00 | 26.18 |
| ATOM | 20 | O | ASP | 823 | 41.419 | 26.091 | 42.569 | 1.00 | 23.19 |
| ATOM | 21 | N | ALA | 824 | 42.414 | 24.414 | 43.635 | 1.00 | 26.01 |
| ATOM | 22 | CA | ALA | 824 | 41.991 | 24.697 | 44.985 | 1.00 | 25.75 |
| ATOM | 23 | CB | ALA | 824 | 42.690 | 23.681 | 45.869 | 1.00 | 25.09 |
| ATOM | 24 | C | ALA | 824 | 42.218 | 26.088 | 45.494 | 1.00 | 24.89 |
| ATOM | 25 | O | ALA | 824 | 41.464 | 26.569 | 46.328 | 1.00 | 23.26 |
| ATOM | 26 | N | SER | 825 | 43.282 | 26.737 | 45.092 | 1.00 | 25.76 |
| ATOM | 27 | CA | SER | 825 | 43.443 | 28.125 | 45.586 | 1.00 | 28.32 |
| ATOM | 28 | CB | SER | 825 | 44.774 | 28.851 | 45.162 | 1.00 | 30.00 |
| ATOM | 29 | OG | SER | 825 | 45.622 | 27.956 | 44.439 | 1.00 | 31.61 |
| ATOM | 30 | C | SER | 825 | 42.335 | 28.979 | 44.998 | 1.00 | 27.13 |
| ATOM | 31 | O | SER | 825 | 41.674 | 29.808 | 45.642 | 1.00 | 28.29 |
| ATOM | 32 | N | LYS | 826 | 42.187 | 28.813 | 43.678 | 1.00 | 25.69 |
| ATOM | 33 | CA | LYS | 826 | 41.163 | 29.603 | 42.968 | 1.00 | 23.69 |
| ATOM | 34 | CB | LYS | 826 | 41.420 | 29.434 | 41.453 | 1.00 | 24.28 |
| ATOM | 35 | CG | LYS | 826 | 40.940 | 30.509 | 40.476 | 1.00 | 26.68 |
| ATOM | 36 | CD | LYS | 826 | 41.980 | 30.465 | 39.301 | 1.00 | 38.26 |
| ATOM | 37 | CE | LYS | 826 | 43.457 | 31.099 | 39.500 | 1.00 | 43.74 |
| ATOM | 38 | NZ | LYS | 826 | 44.330 | 31.077 | 38.281 | 1.00 | 41.93 |
| ATOM | 39 | C | LYS | 826 | 39.755 | 29.174 | 43.399 | 1.00 | 21.18 |
| ATOM | 40 | O | LYS | 826 | 38.967 | 30.010 | 43.789 | 1.00 | 21.93 |
| ATOM | 41 | N | TRP | 827 | 39.434 | 27.903 | 43.497 | 1.00 | 17.48 |
| ATOM | 42 | CA | TRP | 827 | 38.067 | 27.488 | 43.845 | 1.00 | 13.34 |
| ATOM | 43 | CB | TRP | 827 | 37.531 | 26.331 | 42.953 | 1.00 | 12.78 |
| ATOM | 44 | CG | TRP | 827 | 37.876 | 26.645 | 41.584 | 1.00 | 8.55 |
| ATOM | 45 | CD2 | TRP | 827 | 37.204 | 27.641 | 40.933 | 1.00 | 5.18 |
| ATOM | 46 | CE2 | TRP | 827 | 37.914 | 27.841 | 39.724 | 1.00 | 6.96 |
| ATOM | 47 | CE3 | TRP | 827 | 36.071 | 28.353 | 41.317 | 1.00 | 3.14 |
| ATOM | 48 | CD1 | TRP | 827 | 38.968 | 26.249 | 40.777 | 1.00 | 9.15 |
| ATOM | 49 | NE1 | TRP | 827 | 39.000 | 26.993 | 39.632 | 1.00 | 11.49 |
| ATOM | 50 | CZ2 | TRP | 827 | 37.430 | 28.805 | 38.848 | 1.00 | 6.02 |
| ATOM | 51 | CZ3 | TRP | 827 | 35.622 | 29.305 | 40.443 | 1.00 | 4.71 |
| ATOM | 52 | CH2 | TRP | 827 | 36.283 | 29.531 | 39.225 | 1.00 | 7.61 |
| ATOM | 53 | C | TRP | 827 | 37.734 | 27.029 | 45.178 | 1.00 | 15.40 |
| ATOM | 54 | O | TRP | 827 | 36.556 | 27.180 | 45.502 | 1.00 | 20.78 |
| ATOM | 55 | N | GLU | 828 | 38.602 | 26.459 | 46.006 | 1.00 | 17.45 |
| ATOM | 56 | CA | GLU | 828 | 38.078 | 25.936 | 47.296 | 1.00 | 16.73 |
| ATOM | 57 | CB | GLU | 828 | 39.273 | 25.203 | 47.951 | 1.00 | 19.98 |
| ATOM | 58 | CG | GLU | 828 | 39.041 | 24.136 | 49.101 | 1.00 | 19.35 |
| ATOM | 59 | CD | GLU | 828 | 38.218 | 22.950 | 48.676 | 1.00 | 17.22 |
| ATOM | 60 | OE1 | GLU | 828 | 37.526 | 22.380 | 49.511 | 1.00 | 16.17 |
| ATOM | 61 | OE2 | GLU | 828 | 38.297 | 22.590 | 47.510 | 1.00 | 23.53 |
| ATOM | 62 | C | GLU | 828 | 37.393 | 26.926 | 48.222 | 1.00 | 17.05 |
| ATOM | 63 | O | GLU | 828 | 37.814 | 28.054 | 48.412 | 1.00 | 18.57 |
| ATOM | 64 | N | PHE | 829 | 36.322 | 26.523 | 48.883 | 1.00 | 16.94 |
| ATOM | 65 | CA | PHE | 829 | 35.587 | 27.445 | 49.755 | 1.00 | 16.74 |
| ATOM | 66 | CB | PHE | 829 | 34.340 | 27.923 | 48.918 | 1.00 | 15.15 |
| ATOM | 67 | CG | PHE | 829 | 33.379 | 28.816 | 49.623 | 1.00 | 12.24 |
| ATOM | 68 | CD1 | PHE | 829 | 33.702 | 30.164 | 49.831 | 1.00 | 8.89 |
| ATOM | 69 | CD2 | PHE | 829 | 32.159 | 28.285 | 50.109 | 1.00 | 7.57 |
| ATOM | 70 | CE1 | PHE | 829 | 32.786 | 30.972 | 50.531 | 1.00 | 8.96 |
| ATOM | 71 | CE2 | PHE | 829 | 31.249 | 29.091 | 50.813 | 1.00 | 11.13 |
| ATOM | 72 | CZ | PHE | 829 | 31.571 | 30.438 | 51.020 | 1.00 | 11.02 |
| ATOM | 73 | C | PHE | 829 | 35.222 | 26.711 | 51.042 | 1.00 | 19.68 |
| ATOM | 74 | O | PHE | 829 | 34.901 | 25.520 | 50.961 | 1.00 | 19.57 |
| ATOM | 75 | N | PRO | 830 | 35.369 | 27.327 | 52.244 | 1.00 | 20.13 |
| ATOM | 76 | CD | PRO | 830 | 36.227 | 28.481 | 52.588 | 1.00 | 21.27 |
| ATOM | 77 | CA | PRO | 830 | 35.202 | 26.539 | 53.420 | 1.00 | 20.37 |
| ATOM | 78 | CB | PRO | 830 | 35.736 | 27.431 | 54.524 | 1.00 | 17.95 |
| ATOM | 79 | CG | PRO | 830 | 35.757 | 28.791 | 53.965 | 1.00 | 16.28 |
| ATOM | 80 | C | PRO | 830 | 33.809 | 26.089 | 53.608 | 1.00 | 22.74 |
| ATOM | 81 | O | PRO | 830 | 32.934 | 26.940 | 53.689 | 1.00 | 26.96 |
| ATOM | 82 | N | ARG | 831 | 33.574 | 24.787 | 53.775 | 1.00 | 25.54 |
| ATOM | 83 | CA | ARG | 831 | 32.211 | 24.224 | 53.995 | 1.00 | 28.48 |
| ATOM | 84 | CB | ARG | 831 | 32.373 | 22.696 | 54.108 | 1.00 | 30.49 |
| ATOM | 85 | CG | ARG | 831 | 31.250 | 22.048 | 53.264 | 1.00 | 33.51 |
| ATOM | 86 | CD | ARG | 831 | 31.324 | 20.518 | 52.950 | 1.00 | 30.49 |
| ATOM | 87 | NE | ARG | 831 | 32.701 | 20.005 | 52.716 | 1.00 | 29.37 |
| ATOM | 88 | CZ | ARG | 831 | 32.933 | 19.091 | 51.762 | 1.00 | 27.25 |
| ATOM | 89 | NH1 | ARG | 831 | 34.148 | 18.595 | 51.550 | 1.00 | 17.91 |
| ATOM | 90 | NH2 | ARG | 831 | 31.943 | 18.727 | 50.944 | 1.00 | 28.92 |
| ATOM | 91 | C | ARG | 831 | 31.450 | 24.784 | 55.211 | 1.00 | 29.67 |
| ATOM | 92 | O | ARG | 831 | 30.330 | 24.451 | 55.566 | 1.00 | 28.93 |
| ATOM | 93 | N | ASP | 832 | 32.156 | 25.602 | 55.967 | 1.00 | 32.80 |
| ATOM | 94 | CA | ASP | 832 | 31.690 | 26.312 | 57.213 | 1.00 | 35.16 |
| ATOM | 95 | CB | ASP | 832 | 32.889 | 26.387 | 58.333 | 1.00 | 40.94 |
| ATOM | 96 | CG | ASP | 832 | 34.371 | 26.004 | 57.886 | 1.00 | 48.02 |
| ATOM | 97 | OD1 | ASP | 832 | 34.589 | 24.914 | 57.305 | 1.00 | 50.93 |
| ATOM | 98 | OD2 | ASP | 832 | 35.319 | 26.782 | 58.136 | 1.00 | 50.92 |
| ATOM | 99 | C | ASP | 832 | 31.188 | 27.736 | 56.854 | 1.00 | 32.04 |
| ATOM | 100 | O | ASP | 832 | 30.508 | 28.425 | 57.602 | 1.00 | 34.33 |
| ATOM | 101 | N | ARG | 833 | 31.649 | 28.290 | 55.747 | 1.00 | 27.46 |
| ATOM | 102 | CA | ARG | 833 | 31.198 | 29.604 | 55.275 | 1.00 | 22.37 |
| ATOM | 103 | CB | ARG | 833 | 32.446 | 30.112 | 54.559 | 1.00 | 22.63 |
| ATOM | 104 | CG | ARG | 833 | 33.361 | 30.691 | 55.583 | 1.00 | 22.10 |
| ATOM | 105 | CD | ARG | 833 | 33.751 | 32.110 | 55.158 | 1.00 | 27.12 |
| ATOM | 106 | NE | ARG | 833 | 35.102 | 32.284 | 54.563 | 1.00 | 31.23 |
| ATOM | 107 | CZ | ARG | 833 | 36.269 | 31.985 | 55.202 | 1.00 | 33.95 |
| ATOM | 108 | NH1 | ARG | 833 | 37.430 | 32.228 | 54.548 | 1.00 | 34.06 |
| ATOM | 109 | NH2 | ARG | 833 | 36.310 | 31.405 | 56.425 | 1.00 | 31.38 |
| ATOM | 110 | C | ARG | 833 | 29.893 | 29.439 | 54.379 | 1.00 | 21.61 |
| ATOM | 111 | O | ARG | 833 | 29.383 | 30.317 | 53.666 | 1.00 | 21.01 |
| ATOM | 112 | N | LEU | 834 | 29.235 | 28.292 | 54.486 | 1.00 | 18.69 |
| ATOM | 113 | CA | LEU | 834 | 28.051 | 27.993 | 53.702 | 1.00 | 17.25 |
| ATOM | 114 | CB | LEU | 834 | 28.448 | 26.955 | 52.604 | 1.00 | 19.28 |
| ATOM | 115 | CG | LEU | 834 | 27.500 | 26.602 | 51.420 | 1.00 | 13.79 |
| ATOM | 116 | CD1 | LEU | 834 | 27.348 | 27.722 | 50.401 | 1.00 | 12.97 |
| ATOM | 117 | CD2 | LEU | 834 | 28.154 | 25.456 | 50.716 | 1.00 | 11.46 |
| ATOM | 118 | C | LEU | 834 | 26.912 | 27.465 | 54.510 | 1.00 | 17.15 |
| ATOM | 119 | O | LEU | 834 | 26.969 | 26.380 | 55.067 | 1.00 | 20.71 |
| ATOM | 120 | N | LYS | 835 | 25.821 | 28.182 | 54.466 | 1.00 | 16.75 |
| ATOM | 121 | CA | LYS | 835 | 24.601 | 27.860 | 55.188 | 1.00 | 19.33 |
| ATOM | 122 | CB | LYS | 835 | 24.181 | 29.170 | 55.910 | 1.00 | 23.81 |
| ATOM | 123 | CG | LYS | 835 | 25.424 | 29.973 | 56.551 | 1.00 | 30.13 |
| ATOM | 124 | CD | LYS | 835 | 25.645 | 31.518 | 56.318 | 1.00 | 24.89 |
| ATOM | 125 | CE | LYS | 835 | 24.416 | 32.290 | 56.931 | 1.00 | 29.97 |
| ATOM | 126 | NZ | LYS | 835 | 24.353 | 33.721 | 56.542 | 1.00 | 24.63 |
| ATOM | 127 | C | LYS | 835 | 23.499 | 27.280 | 54.280 | 1.00 | 22.20 |
| ATOM | 128 | O | LYS | 835 | 22.722 | 27.986 | 53.639 | 1.00 | 22.84 |
| ATOM | 129 | N | LEU | 836 | 23.477 | 25.946 | 54.115 | 1.00 | 22.63 |
| ATOM | 130 | CA | LEU | 836 | 22.484 | 25.225 | 53.300 | 1.00 | 18.76 |
| ATOM | 131 | CB | LEU | 836 | 22.877 | 23.758 | 53.352 | 1.00 | 17.39 |
| ATOM | 132 | CG | LEU | 836 | 23.813 | 23.298 | 52.252 | 1.00 | 17.49 |
| ATOM | 133 | CD1 | LEU | 836 | 24.914 | 24.274 | 51.907 | 1.00 | 18.83 |
| ATOM | 134 | CD2 | LEU | 836 | 24.420 | 22.010 | 52.769 | 1.00 | 17.99 |
| ATOM | 135 | C | LEU | 836 | 21.008 | 25.400 | 53.674 | 1.00 | 19.41 |
| ATOM | 136 | O | LEU | 836 | 20.690 | 25.528 | 54.835 | 1.00 | 19.27 |
| ATOM | 137 | N | GLY | 837 | 20.045 | 25.360 | 52.748 | 1.00 | 19.98 |
| ATOM | 138 | CA | GLY | 837 | 18.617 | 25.515 | 53.098 | 1.00 | 16.05 |
| ATOM | 139 | C | GLY | 837 | 17.731 | 24.615 | 52.268 | 1.00 | 16.52 |
| ATOM | 140 | O | GLY | 837 | 17.964 | 23.417 | 52.076 | 1.00 | 16.42 |
| ATOM | 141 | N | LYS | 838 | 16.648 | 25.185 | 51.784 | 1.00 | 16.25 |
| ATOM | 142 | CA | LYS | 838 | 15.686 | 24.478 | 50.954 | 1.00 | 16.53 |
| ATOM | 143 | CB | LYS | 838 | 14.491 | 25.367 | 50.696 | 1.00 | 20.17 |
| ATOM | 144 | CG | LYS | 838 | 13.631 | 25.327 | 51.954 | 1.00 | 28.06 |
| ATOM | 145 | CD | LYS | 838 | 12.210 | 25.929 | 51.705 | 1.00 | 33.84 |
| ATOM | 146 | CE | LYS | 838 | 11.039 | 24.898 | 51.629 | 1.00 | 38.61 |
| ATOM | 147 | NZ | LYS | 838 | 9.841 | 25.436 | 50.929 | 1.00 | 42.47 |
| ATOM | 148 | C | LYS | 838 | 16.228 | 24.000 | 49.593 | 1.00 | 19.63 |
| ATOM | 149 | O | LYS | 838 | 17.077 | 24.600 | 48.886 | 1.00 | 17.81 |
| ATOM | 150 | N | PRO | 839 | 15.703 | 22.852 | 49.164 | 1.00 | 19.44 |
| ATOM | 151 | CD | PRO | 839 | 14.844 | 21.923 | 49.920 | 1.00 | 18.61 |
| ATOM | 152 | CA | PRO | 839 | 16.105 | 22.336 | 47.864 | 1.00 | 18.05 |
| ATOM | 153 | CB | PRO | 839 | 15.864 | 20.870 | 48.025 | 1.00 | 19.73 |
| ATOM | 154 | CG | PRO | 839 | 14.567 | 20.902 | 48.836 | 1.00 | 21.35 |
| ATOM | 155 | C | PRO | 839 | 15.458 | 22.951 | 46.609 | 1.00 | 17.23 |
| ATOM | 156 | O | PRO | 839 | 14.260 | 23.237 | 46.524 | 1.00 | 14.70 |
| ATOM | 157 | N | LEU | 840 | 16.368 | 23.308 | 45.716 | 1.00 | 14.39 |
| ATOM | 158 | CA | LEU | 840 | 16.042 | 23.850 | 44.446 | 1.00 | 14.10 |
| ATOM | 159 | CB | LEU | 840 | 17.267 | 24.548 | 43.899 | 1.00 | 11.14 |
| ATOM | 160 | CG | LEU | 840 | 17.378 | 25.753 | 44.772 | 1.00 | 9.22 |
| ATOM | 161 | CD1 | LEU | 840 | 18.527 | 26.581 | 44.266 | 1.00 | 6.51 |
| ATOM | 162 | CD2 | LEU | 840 | 16.070 | 26.564 | 44.742 | 1.00 | 6.14 |
| ATOM | 163 | C | LEU | 840 | 15.609 | 22.654 | 43.641 | 1.00 | 17.84 |
| ATOM | 164 | O | LEU | 840 | 14.514 | 22.681 | 43.111 | 1.00 | 23.74 |
| ATOM | 165 | N | GLY | 841 | 16.362 | 21.567 | 43.537 | 1.00 | 19.92 |
| ATOM | 166 | CA | GLY | 841 | 15.891 | 20.373 | 42.811 | 1.00 | 16.74 |
| ATOM | 167 | C | GLY | 841 | 16.411 | 19.051 | 43.410 | 1.00 | 20.37 |
| ATOM | 168 | O | GLY | 841 | 17.443 | 18.976 | 44.097 | 1.00 | 17.04 |
| ATOM | 169 | N | ARG | 842 | 15.680 | 17.963 | 43.192 | 1.00 | 22.51 |
| ATOM | 170 | CA | ARG | 842 | 16.130 | 16.651 | 43.684 | 1.00 | 26.91 |
| ATOM | 171 | CB | ARG | 842 | 15.484 | 16.257 | 44.998 | 1.00 | 30.90 |
| ATOM | 172 | CG | ARG | 842 | 16.100 | 17.108 | 46.066 | 1.00 | 41.70 |
| ATOM | 173 | CD | ARG | 842 | 15.653 | 16.653 | 47.454 | 1.00 | 52.08 |
| ATOM | 174 | NE | ARG | 842 | 16.292 | 15.361 | 47.812 | 1.00 | 60.24 |
| ATOM | 175 | CZ | ARG | 842 | 16.454 | 14.998 | 49.097 | 1.00 | 63.35 |
| ATOM | 176 | NH1 | ARG | 842 | 17.012 | 13.820 | 49.380 | 1.00 | 64.81 |
| ATOM | 177 | NH2 | ARG | 842 | 16.073 | 15.808 | 50.101 | 1.00 | 66.69 |
| ATOM | 178 | C | ARG | 842 | 15.857 | 15.472 | 42.749 | 1.00 | 26.75 |
| ATOM | 179 | O | ARG | 842 | 14.791 | 15.286 | 42.158 | 1.00 | 27.66 |
| ATOM | 180 | N | GLY | 843 | 16.835 | 14.603 | 42.657 | 1.00 | 25.95 |
| ATOM | 181 | CA | GLY | 843 | 16.729 | 13.404 | 41.814 | 1.00 | 23.80 |
| ATOM | 182 | C | GLY | 843 | 16.934 | 12.216 | 42.721 | 1.00 | 23.54 |
| ATOM | 183 | O | GLY | 843 | 16.566 | 12.320 | 43.893 | 1.00 | 22.08 |
| ATOM | 184 | N | ALA | 844 | 17.454 | 11.091 | 42.203 | 1.00 | 23.39 |
| ATOM | 185 | CA | ALA | 844 | 17.727 | 9.848 | 42.999 | 1.00 | 26.79 |
| ATOM | 186 | CB | ALA | 844 | 17.759 | 8.539 | 42.140 | 1.00 | 24.80 |
| ATOM | 187 | C | ALA | 844 | 19.069 | 9.824 | 43.738 | 1.00 | 27.40 |
| ATOM | 188 | O | ALA | 844 | 19.257 | 9.308 | 44.837 | 1.00 | 28.99 |
| ATOM | 189 | N | PHE | 845 | 20.044 | 10.412 | 43.095 | 1.00 | 26.61 |
| ATOM | 190 | CA | PHE | 845 | 21.414 | 10.447 | 43.607 | 1.00 | 25.81 |
| ATOM | 191 | CB | PHE | 845 | 22.307 | 9.578 | 42.705 | 1.00 | 29.43 |
| ATOM | 192 | CG | PHE | 845 | 21.741 | 8.241 | 42.458 | 1.00 | 30.41 |
| ATOM | 193 | CD1 | PHE | 845 | 21.883 | 7.676 | 41.187 | 1.00 | 30.74 |
| ATOM | 194 | CD2 | PHE | 845 | 21.084 | 7.519 | 43.474 | 1.00 | 33.15 |
| ATOM | 195 | CE1 | PHE | 845 | 21.376 | 6.393 | 40.924 | 1.00 | 31.81 |
| ATOM | 196 | CE2 | PHE | 845 | 20.568 | 6.232 | 43.222 | 1.00 | 35.44 |
| ATOM | 197 | CZ | PHE | 845 | 20.723 | 5.675 | 41.935 | 1.00 | 33.07 |
| ATOM | 198 | C | PHE | 845 | 21.983 | 11.835 | 43.649 | 1.00 | 23.22 |
| ATOM | 199 | O | PHE | 845 | 23.167 | 12.011 | 43.937 | 1.00 | 22.82 |
| ATOM | 200 | N | GLY | 846 | 21.176 | 12.838 | 43.338 | 1.00 | 19.50 |
| ATOM | 201 | CA | GLY | 846 | 21.768 | 14.169 | 43.394 | 1.00 | 16.06 |
| ATOM | 202 | C | GLY | 846 | 20.773 | 15.148 | 43.844 | 1.00 | 14.22 |
| ATOM | 203 | O | GLY | 846 | 19.604 | 14.792 | 43.996 | 1.00 | 14.73 |
| ATOM | 204 | N | GLN | 847 | 21.221 | 16.357 | 44.081 | 1.00 | 10.40 |
| ATOM | 205 | CA | GLN | 847 | 20.285 | 17.396 | 44.490 | 1.00 | 12.42 |
| ATOM | 206 | CB | GLN | 847 | 19.865 | 17.225 | 45.952 | 1.00 | 12.40 |
| ATOM | 207 | CG | GLN | 847 | 20.927 | 17.564 | 47.026 | 1.00 | 15.34 |
| ATOM | 208 | CD | GLN | 847 | 20.501 | 17.121 | 48.405 | 1.00 | 14.41 |
| ATOM | 209 | OE1 | GLN | 847 | 19.382 | 17.323 | 48.882 | 1.00 | 20.01 |
| ATOM | 210 | NE2 | GLN | 847 | 21.388 | 16.473 | 49.092 | 1.00 | 13.81 |
| ATOM | 211 | C | GLN | 847 | 20.898 | 18.756 | 44.346 | 1.00 | 12.11 |
| ATOM | 212 | O | GLN | 847 | 22.121 | 18.841 | 44.324 | 1.00 | 16.25 |
| ATOM | 213 | N | VAL | 848 | 20.093 | 19.793 | 44.227 | 1.00 | 11.09 |
| ATOM | 214 | CA | VAL | 848 | 20.509 | 21.199 | 44.142 | 1.00 | 11.33 |
| ATOM | 215 | CB | VAL | 848 | 20.158 | 21.925 | 42.822 | 1.00 | 10.65 |
| ATOM | 216 | CG1 | VAL | 848 | 20.752 | 23.329 | 42.801 | 1.00 | 7.14 |
| ATOM | 217 | CG2 | VAL | 848 | 20.862 | 21.212 | 41.634 | 1.00 | 12.03 |
| ATOM | 218 | C | VAL | 848 | 19.727 | 21.873 | 45.227 | 1.00 | 14.23 |
| ATOM | 219 | O | VAL | 848 | 18.480 | 21.875 | 45.311 | 1.00 | 11.55 |
| ATOM | 220 | N | ILE | 849 | 20.527 | 22.296 | 46.193 | 1.00 | 16.01 |
| ATOM | 221 | CA | ILE | 849 | 19.965 | 22.980 | 47.374 | 1.00 | 17.85 |
| ATOM | 222 | CB | ILE | 849 | 20.283 | 22.101 | 48.685 | 1.00 | 16.31 |
| ATOM | 223 | CG2 | ILE | 849 | 21.670 | 21.499 | 48.756 | 1.00 | 16.01 |
| ATOM | 224 | CG1 | ILE | 849 | 20.158 | 23.019 | 49.869 | 1.00 | 18.43 |
| ATOM | 225 | CD1 | ILE | 849 | 20.434 | 22.290 | 51.164 | 1.00 | 24.30 |
| ATOM | 226 | C | ILE | 849 | 20.445 | 24.432 | 47.492 | 1.00 | 17.95 |
| ATOM | 227 | O | ILE | 849 | 21.555 | 24.818 | 47.127 | 1.00 | 17.26 |
| ATOM | 228 | N | GLU | 850 | 19.524 | 25.309 | 47.854 | 1.00 | 19.59 |
| ATOM | 229 | CA | GLU | 850 | 19.844 | 26.768 | 48.033 | 1.00 | 18.60 |
| ATOM | 230 | CB | GLU | 850 | 18.514 | 27.553 | 48.180 | 1.00 | 18.16 |
| ATOM | 231 | CG | GLU | 850 | 18.626 | 29.007 | 47.739 | 1.00 | 16.89 |
| ATOM | 232 | CD | GLU | 850 | 17.315 | 29.770 | 47.855 | 1.00 | 15.62 |
| ATOM | 233 | OE1 | GLU | 850 | 16.246 | 29.234 | 48.116 | 1.00 | 19.12 |
| ATOM | 234 | OE2 | GLU | 850 | 17.352 | 30.971 | 47.681 | 1.00 | 21.59 |
| ATOM | 235 | C | GLU | 850 | 20.711 | 27.003 | 49.262 | 1.00 | 15.71 |
| ATOM | 236 | O | GLU | 850 | 20.515 | 26.381 | 50.287 | 1.00 | 15.36 |
| ATOM | 237 | N | ALA | 851 | 21.545 | 27.989 | 49.272 | 1.00 | 16.19 |
| ATOM | 238 | CA | ALA | 851 | 22.406 | 28.177 | 50.440 | 1.00 | 16.18 |
| ATOM | 239 | CB | ALA | 851 | 23.674 | 27.320 | 50.327 | 1.00 | 10.56 |
| ATOM | 240 | C | ALA | 851 | 22.854 | 29.613 | 50.574 | 1.00 | 16.96 |
| ATOM | 241 | O | ALA | 851 | 22.647 | 30.395 | 49.652 | 1.00 | 20.19 |
| ATOM | 242 | N | ASP | 852 | 23.422 | 30.009 | 51.700 | 1.00 | 15.39 |
| ATOM | 243 | CA | ASP | 852 | 23.902 | 31.363 | 51.788 | 1.00 | 12.86 |
| ATOM | 244 | CB | ASP | 852 | 23.390 | 32.128 | 53.040 | 1.00 | 16.30 |
| ATOM | 245 | CG | ASP | 852 | 21.912 | 32.518 | 53.153 | 1.00 | 22.97 |
| ATOM | 246 | OD1 | ASP | 852 | 21.353 | 32.570 | 54.266 | 1.00 | 25.60 |
| ATOM | 247 | OD2 | ASP | 852 | 21.310 | 32.771 | 52.117 | 1.00 | 27.08 |
| ATOM | 248 | C | ASP | 852 | 25.370 | 31.274 | 51.872 | 1.00 | 10.50 |
| ATOM | 249 | O | ASP | 852 | 25.883 | 30.714 | 52.809 | 1.00 | 12.87 |
| ATOM | 250 | N | ALA | 853 | 26.119 | 31.825 | 50.983 | 1.00 | 11.10 |
| ATOM | 251 | CA | ALA | 853 | 27.555 | 31.776 | 51.060 | 1.00 | 13.76 |
| ATOM | 252 | CB | ALA | 853 | 28.094 | 31.326 | 49.707 | 1.00 | 14.30 |
| ATOM | 253 | C | ALA | 853 | 28.167 | 33.133 | 51.442 | 1.00 | 18.59 |
| ATOM | 254 | O | ALA | 853 | 27.991 | 34.159 | 50.758 | 1.00 | 19.95 |
| ATOM | 255 | N | PHE | 854 | 28.938 | 33.155 | 52.547 | 1.00 | 21.31 |
| ATOM | 256 | CA | PHE | 854 | 29.584 | 34.396 | 53.065 | 1.00 | 20.99 |
| ATOM | 257 | CB | PHE | 854 | 29.778 | 34.278 | 54.656 | 1.00 | 22.36 |
| ATOM | 258 | CG | PHE | 854 | 30.310 | 35.558 | 55.249 | 1.00 | 19.94 |
| ATOM | 259 | CD1 | PHE | 854 | 31.688 | 35.859 | 55.278 | 1.00 | 20.28 |
| ATOM | 260 | CD2 | PHE | 854 | 29.420 | 36.522 | 55.759 | 1.00 | 17.93 |
| ATOM | 261 | CE1 | PHE | 854 | 32.182 | 37.089 | 55.790 | 1.00 | 19.96 |
| ATOM | 262 | CE2 | PHE | 854 | 29.892 | 37.745 | 56.274 | 1.00 | 19.13 |
| ATOM | 263 | CZ | PHE | 854 | 31.270 | 38.024 | 56.284 | 1.00 | 16.96 |
| ATOM | 264 | C | PHE | 854 | 30.905 | 34.752 | 52.398 | 1.00 | 18.33 |
| ATOM | 265 | O | PHE | 854 | 31.901 | 34.129 | 52.583 | 1.00 | 19.13 |
| ATOM | 266 | N | GLY | 855 | 31.051 | 35.755 | 51.632 | 1.00 | 20.22 |
| ATOM | 267 | CA | GLY | 855 | 32.336 | 36.024 | 51.030 | 1.00 | 22.59 |
| ATOM | 268 | C | GLY | 855 | 32.787 | 35.038 | 49.985 | 1.00 | 26.51 |
| ATOM | 269 | O | GLY | 855 | 34.009 | 34.951 | 49.739 | 1.00 | 27.80 |
| ATOM | 270 | N | ILE | 856 | 31.852 | 34.380 | 49.243 | 1.00 | 28.23 |
| ATOM | 271 | CA | ILE | 856 | 32.311 | 33.434 | 48.172 | 1.00 | 28.36 |
| ATOM | 272 | CB | ILE | 856 | 31.117 | 32.506 | 47.770 | 1.00 | 24.45 |
| ATOM | 273 | CG2 | ILE | 856 | 29.927 | 33.240 | 47.270 | 1.00 | 19.45 |
| ATOM | 274 | CG1 | ILE | 856 | 31.695 | 31.492 | 46.774 | 1.00 | 22.95 |
| ATOM | 275 | CD1 | ILE | 856 | 30.831 | 30.235 | 46.769 | 1.00 | 21.31 |
| ATOM | 276 | C | ILE | 856 | 32.926 | 34.137 | 46.940 | 1.00 | 32.59 |
| ATOM | 277 | O | ILE | 856 | 33.921 | 33.745 | 46.344 | 1.00 | 33.97 |
| ATOM | 278 | N | ASP | 857 | 32.315 | 35.261 | 46.613 | 1.00 | 35.81 |
| ATOM | 279 | CA | ASP | 857 | 32.605 | 36.200 | 45.526 | 1.00 | 38.96 |
| ATOM | 280 | CB | ASP | 857 | 31.199 | 36.814 | 45.133 | 1.00 | 42.25 |
| ATOM | 281 | CG | ASP | 857 | 30.343 | 37.561 | 46.241 | 1.00 | 44.81 |
| ATOM | 282 | OD1 | ASP | 857 | 29.352 | 38.224 | 45.838 | 1.00 | 42.34 |
| ATOM | 283 | OD2 | ASP | 857 | 30.633 | 37.448 | 47.467 | 1.00 | 47.52 |
| ATOM | 284 | C | ASP | 857 | 33.664 | 37.309 | 45.789 | 1.00 | 42.76 |
| ATOM | 285 | O | ASP | 857 | 33.583 | 38.420 | 45.269 | 1.00 | 43.54 |
| ATOM | 286 | N | LYS | 858 | 34.682 | 37.135 | 46.604 | 1.00 | 46.91 |
| ATOM | 287 | CA | LYS | 858 | 35.743 | 38.193 | 46.849 | 1.00 | 53.41 |
| ATOM | 288 | CB | LYS | 858 | 36.546 | 38.367 | 45.471 | 1.00 | 59.69 |
| ATOM | 289 | CG | LYS | 858 | 37.512 | 37.157 | 45.096 | 1.00 | 67.88 |
| ATOM | 290 | CD | LYS | 858 | 38.187 | 36.930 | 43.655 | 1.00 | 70.92 |
| ATOM | 291 | CE | LYS | 858 | 39.646 | 36.254 | 43.750 | 1.00 | 73.51 |
| ATOM | 292 | NZ | LYS | 858 | 40.096 | 35.388 | 42.619 | 1.00 | 70.67 |
| ATOM | 293 | C | LYS | 858 | 35.278 | 39.592 | 47.447 | 1.00 | 53.66 |
| ATOM | 294 | O | LYS | 858 | 35.741 | 40.739 | 47.204 | 1.00 | 53.43 |
| ATOM | 295 | N | THR | 859 | 34.472 | 39.395 | 48.500 | 1.00 | 49.30 |
| ATOM | 296 | CA | THR | 859 | 33.824 | 40.455 | 49.294 | 1.00 | 43.47 |
| ATOM | 297 | CB | THR | 859 | 32.465 | 40.781 | 48.660 | 1.00 | 43.40 |
| ATOM | 298 | OG1 | THR | 859 | 31.692 | 39.572 | 48.813 | 1.00 | 40.83 |
| ATOM | 299 | CG2 | THR | 859 | 32.523 | 41.219 | 47.168 | 1.00 | 38.96 |
| ATOM | 300 | C | THR | 859 | 33.568 | 39.912 | 50.698 | 1.00 | 40.61 |
| ATOM | 301 | O | THR | 859 | 33.697 | 38.709 | 50.863 | 1.00 | 43.68 |
| ATOM | 302 | N | ALA | 860 | 33.361 | 40.629 | 51.793 | 1.00 | 33.76 |
| ATOM | 303 | CA | ALA | 860 | 32.994 | 39.858 | 53.003 | 1.00 | 28.46 |
| ATOM | 304 | CB | ALA | 860 | 33.631 | 40.548 | 54.176 | 1.00 | 26.80 |
| ATOM | 305 | C | ALA | 860 | 31.410 | 39.919 | 53.043 | 1.00 | 27.52 |
| ATOM | 306 | O | ALA | 860 | 30.683 | 40.169 | 54.027 | 1.00 | 25.01 |
| ATOM | 307 | N | THR | 861 | 30.814 | 39.746 | 51.846 | 1.00 | 26.00 |
| ATOM | 308 | CA | THR | 861 | 29.334 | 39.789 | 51.579 | 1.00 | 22.13 |
| ATOM | 309 | CB | THR | 861 | 28.989 | 40.388 | 50.168 | 1.00 | 19.09 |
| ATOM | 310 | OG1 | THR | 861 | 29.847 | 41.493 | 49.978 | 1.00 | 15.19 |
| ATOM | 311 | CG2 | THR | 861 | 27.506 | 40.804 | 49.992 | 1.00 | 14.82 |
| ATOM | 312 | C | THR | 861 | 28.624 | 38.438 | 51.592 | 1.00 | 22.11 |
| ATOM | 313 | O | THR | 861 | 29.162 | 37.474 | 51.043 | 1.00 | 22.99 |
| ATOM | 314 | N | CYS | 862 | 27.411 | 38.371 | 52.179 | 1.00 | 22.40 |
| ATOM | 315 | CA | CYS | 862 | 26.608 | 37.119 | 52.173 | 1.00 | 22.73 |
| ATOM | 316 | CB | CYS | 862 | 25.336 | 37.056 | 53.044 | 1.00 | 18.59 |
| ATOM | 317 | SG | CYS | 862 | 24.815 | 35.338 | 53.494 | 1.00 | 26.93 |
| ATOM | 318 | C | CYS | 862 | 26.086 | 37.121 | 50.794 | 1.00 | 22.95 |
| ATOM | 319 | O | CYS | 862 | 25.811 | 38.203 | 50.260 | 1.00 | 26.48 |
| ATOM | 320 | N | ARG | 863 | 25.985 | 35.971 | 50.174 | 1.00 | 24.36 |
| ATOM | 321 | CA | ARG | 863 | 25.469 | 35.922 | 48.797 | 1.00 | 22.79 |
| ATOM | 322 | CB | ARG | 863 | 26.719 | 36.265 | 48.001 | 1.00 | 22.28 |
| ATOM | 323 | CG | ARG | 863 | 27.217 | 35.371 | 46.914 | 1.00 | 28.94 |
| ATOM | 324 | CD | ARG | 863 | 26.218 | 35.159 | 45.768 | 1.00 | 37.53 |
| ATOM | 325 | NE | ARG | 863 | 26.697 | 35.722 | 44.486 | 1.00 | 42.79 |
| ATOM | 326 | CZ | ARG | 863 | 26.015 | 35.598 | 43.326 | 1.00 | 46.47 |
| ATOM | 327 | NH1 | ARG | 863 | 26.609 | 36.182 | 42.278 | 1.00 | 47.72 |
| ATOM | 328 | NH2 | ARG | 863 | 24.821 | 34.943 | 43.200 | 1.00 | 43.35 |
| ATOM | 329 | C | ARG | 863 | 24.805 | 34.570 | 48.558 | 1.00 | 19.98 |
| ATOM | 330 | O | ARG | 863 | 25.310 | 33.560 | 49.030 | 1.00 | 20.52 |
| ATOM | 331 | N | THR | 864 | 23.575 | 34.558 | 48.067 | 1.00 | 15.49 |
| ATOM | 332 | CA | THR | 864 | 22.802 | 33.352 | 47.764 | 1.00 | 14.56 |
| ATOM | 333 | CB | THR | 864 | 21.364 | 33.670 | 47.435 | 1.00 | 14.48 |
| ATOM | 334 | OG1 | THR | 864 | 20.980 | 34.631 | 48.384 | 1.00 | 17.98 |
| ATOM | 335 | CG2 | THR | 864 | 20.371 | 32.510 | 47.575 | 1.00 | 13.91 |
| ATOM | 336 | C | THR | 864 | 23.357 | 32.623 | 46.529 | 1.00 | 16.34 |
| ATOM | 337 | O | THR | 864 | 23.507 | 33.154 | 45.401 | 1.00 | 19.29 |
| ATOM | 338 | N | VAL | 865 | 23.602 | 31.332 | 46.759 | 1.00 | 13.52 |
| ATOM | 339 | CA | VAL | 865 | 24.106 | 30.447 | 45.747 | 1.00 | 9.03 |
| ATOM | 340 | CB | VAL | 865 | 25.586 | 30.099 | 46.118 | 1.00 | 7.73 |
| ATOM | 341 | CG1 | VAL | 865 | 26.372 | 31.438 | 46.218 | 1.00 | 2.65 |
| ATOM | 342 | CG2 | VAL | 865 | 25.640 | 29.160 | 47.349 | 1.00 | 2.00 |
| ATOM | 343 | C | VAL | 865 | 23.194 | 29.164 | 45.660 | 1.00 | 11.01 |
| ATOM | 344 | O | VAL | 865 | 22.219 | 28.989 | 46.434 | 1.00 | 10.30 |
| ATOM | 345 | N | ALA | 866 | 23.431 | 28.336 | 44.610 | 1.00 | 7.90 |
| ATOM | 346 | CA | ALA | 866 | 22.744 | 27.070 | 44.396 | 1.00 | 6.58 |
| ATOM | 347 | CB | ALA | 866 | 22.156 | 26.870 | 42.997 | 1.00 | 7.22 |
| ATOM | 348 | C | ALA | 866 | 23.880 | 26.119 | 44.465 | 1.00 | 9.38 |
| ATOM | 349 | O | ALA | 866 | 25.006 | 26.341 | 43.989 | 1.00 | 11.30 |
| ATOM | 350 | N | VAL | 867 | 23.690 | 25.025 | 45.133 | 1.00 | 12.23 |
| ATOM | 351 | CA | VAL | 867 | 24.838 | 24.069 | 45.210 | 1.00 | 13.25 |
| ATOM | 352 | CB | VAL | 867 | 25.669 | 24.244 | 46.679 | 1.00 | 9.85 |
| ATOM | 353 | CG1 | VAL | 867 | 24.905 | 25.059 | 47.653 | 1.00 | 2.00 |
| ATOM | 354 | CG2 | VAL | 867 | 26.076 | 22.920 | 47.225 | 1.00 | 2.00 |
| ATOM | 355 | C | VAL | 867 | 24.440 | 22.630 | 44.937 | 1.00 | 11.42 |
| ATOM | 356 | O | VAL | 867 | 23.454 | 22.105 | 45.451 | 1.00 | 12.66 |
| ATOM | 357 | N | LYS | 868 | 25.144 | 22.055 | 43.983 | 1.00 | 9.59 |
| ATOM | 358 | CA | LYS | 868 | 24.897 | 20.657 | 43.598 | 1.00 | 11.89 |
| ATOM | 359 | CB | LYS | 868 | 25.353 | 20.260 | 42.158 | 1.00 | 11.88 |
| ATOM | 360 | CG | LYS | 868 | 24.733 | 18.923 | 41.719 | 1.00 | 11.67 |
| ATOM | 361 | CD | LYS | 868 | 24.953 | 18.573 | 40.245 | 1.00 | 11.83 |
| ATOM | 362 | CE | LYS | 868 | 23.956 | 19.199 | 39.287 | 1.00 | 10.73 |
| ATOM | 363 | NZ | LYS | 868 | 24.677 | 19.077 | 38.032 | 1.00 | 11.06 |
| ATOM | 364 | C | LYS | 868 | 25.637 | 19.724 | 44.427 | 1.00 | 13.15 |
| ATOM | 365 | O | LYS | 868 | 26.850 | 19.822 | 44.548 | 1.00 | 16.62 |
| ATOM | 366 | N | MET | 869 | 24.967 | 18.747 | 44.926 | 1.00 | 17.71 |
| ATOM | 367 | CA | MET | 869 | 25.676 | 17.733 | 45.747 | 1.00 | 19.80 |
| ATOM | 368 | CB | MET | 869 | 25.809 | 18.261 | 47.192 | 1.00 | 17.51 |
| ATOM | 369 | CG | MET | 869 | 24.409 | 18.565 | 47.748 | 1.00 | 28.59 |
| ATOM | 370 | SD | MET | 869 | 24.291 | 19.396 | 49.371 | 1.00 | 35.80 |
| ATOM | 371 | CE | MET | 869 | 24.961 | 17.951 | 50.256 | 1.00 | 34.49 |
| ATOM | 372 | C | MET | 869 | 24.876 | 16.413 | 45.676 | 1.00 | 17.93 |
| ATOM | 373 | O | MET | 869 | 23.795 | 16.357 | 45.082 | 1.00 | 16.44 |
| ATOM | 374 | N | LEU | 870 | 25.399 | 15.332 | 46.240 | 1.00 | 19.18 |
| ATOM | 375 | CA | LEU | 870 | 24.718 | 14.039 | 46.219 | 1.00 | 19.06 |
| ATOM | 376 | CB | LEU | 870 | 25.755 | 12.902 | 46.255 | 1.00 | 12.92 |
| ATOM | 377 | CG | LEU | 870 | 26.693 | 13.038 | 45.022 | 1.00 | 16.50 |
| ATOM | 378 | CD1 | LEU | 870 | 27.850 | 12.147 | 45.278 | 1.00 | 12.10 |
| ATOM | 379 | CD2 | LEU | 870 | 25.989 | 12.733 | 43.631 | 1.00 | 16.41 |
| ATOM | 380 | C | LEU | 870 | 23.703 | 13.833 | 47.302 | 1.00 | 22.25 |
| ATOM | 381 | O | LEU | 870 | 23.469 | 14.629 | 48.217 | 1.00 | 26.50 |
| ATOM | 382 | N | LYS | 871 | 23.065 | 12.704 | 47.147 | 1.00 | 23.18 |
| ATOM | 383 | CA | LYS | 871 | 22.034 | 12.256 | 48.048 | 1.00 | 22.67 |
| ATOM | 384 | CB | LYS | 871 | 20.709 | 12.161 | 47.292 | 1.00 | 23.83 |
| ATOM | 385 | CG | LYS | 871 | 19.765 | 13.307 | 47.137 | 1.00 | 25.73 |
| ATOM | 386 | CD | LYS | 871 | 18.479 | 12.830 | 46.470 | 1.00 | 30.12 |
| ATOM | 387 | CE | LYS | 871 | 17.896 | 11.498 | 47.018 | 1.00 | 34.14 |
| ATOM | 388 | NZ | LYS | 871 | 16.475 | 11.293 | 46.594 | 1.00 | 39.66 |
| ATOM | 389 | C | LYS | 871 | 22.409 | 10.856 | 48.471 | 1.00 | 24.48 |
| ATOM | 390 | O | LYS | 871 | 23.201 | 10.175 | 47.813 | 1.00 | 21.82 |
| ATOM | 391 | N | GLU | 872 | 21.799 | 10.349 | 49.553 | 1.00 | 28.40 |
| ATOM | 392 | CA | GLU | 872 | 22.126 | 8.954 | 49.875 | 1.00 | 29.30 |
| ATOM | 393 | CB | GLU | 872 | 21.425 | 8.507 | 51.198 | 1.00 | 30.78 |
| ATOM | 394 | CG | GLU | 872 | 22.525 | 7.519 | 51.857 | 1.00 | 38.75 |
| ATOM | 395 | CD | GLU | 872 | 24.013 | 8.100 | 51.947 | 1.00 | 45.91 |
| ATOM | 396 | OE1 | GLU | 872 | 24.815 | 8.079 | 50.951 | 1.00 | 45.36 |
| ATOM | 397 | OE2 | GLU | 872 | 24.341 | 8.586 | 53.054 | 1.00 | 47.86 |
| ATOM | 398 | C | GLU | 872 | 21.736 | 8.036 | 48.703 | 1.00 | 25.81 |
| ATOM | 399 | O | GLU | 872 | 20.761 | 8.284 | 48.024 | 1.00 | 28.71 |
| ATOM | 400 | N | GLY | 873 | 22.397 | 6.939 | 48.426 | 1.00 | 24.43 |
| ATOM | 401 | CA | GLY | 873 | 21.997 | 6.148 | 47.254 | 1.00 | 20.71 |
| ATOM | 402 | C | GLY | 873 | 23.114 | 6.367 | 46.289 | 1.00 | 22.81 |
| ATOM | 403 | O | GLY | 873 | 23.565 | 5.483 | 45.546 | 1.00 | 24.53 |
| ATOM | 404 | N | ALA | 874 | 23.619 | 7.598 | 46.313 | 1.00 | 21.14 |
| ATOM | 405 | CA | ALA | 874 | 24.724 | 7.870 | 45.435 | 1.00 | 19.95 |
| ATOM | 406 | CB | ALA | 874 | 25.204 | 9.288 | 45.625 | 1.00 | 25.40 |
| ATOM | 407 | C | ALA | 874 | 25.835 | 6.935 | 45.790 | 1.00 | 18.22 |
| ATOM | 408 | O | ALA | 874 | 25.837 | 6.341 | 46.841 | 1.00 | 15.23 |
| ATOM | 409 | N | THR | 875 | 26.813 | 6.807 | 44.938 | 1.00 | 17.56 |
| ATOM | 410 | CA | THR | 875 | 27.924 | 5.921 | 45.229 | 1.00 | 19.35 |
| ATOM | 411 | CB | THR | 875 | 27.751 | 4.504 | 44.588 | 1.00 | 21.50 |
| ATOM | 412 | OG1 | THR | 875 | 29.043 | 3.849 | 44.849 | 1.00 | 31.77 |
| ATOM | 413 | CG2 | THR | 875 | 27.377 | 4.461 | 43.084 | 1.00 | 19.53 |
| ATOM | 414 | C | THR | 875 | 29.160 | 6.567 | 44.719 | 1.00 | 19.06 |
| ATOM | 415 | O | THR | 875 | 29.065 | 7.590 | 44.060 | 1.00 | 21.38 |
| ATOM | 416 | N | HIS | 876 | 30.360 | 6.077 | 44.951 | 1.00 | 17.65 |
| ATOM | 417 | CA | HIS | 876 | 31.534 | 6.772 | 44.466 | 1.00 | 14.58 |
| ATOM | 418 | CB | HIS | 876 | 32.698 | 5.905 | 44.811 | 1.00 | 11.66 |
| ATOM | 419 | CG | HIS | 876 | 33.883 | 6.697 | 44.549 | 1.00 | 11.47 |
| ATOM | 420 | CD2 | HIS | 876 | 34.243 | 7.966 | 45.014 | 1.00 | 10.21 |
| ATOM | 421 | ND1 | HIS | 876 | 34.888 | 6.273 | 43.737 | 1.00 | 14.14 |
| ATOM | 422 | CE1 | HIS | 876 | 35.847 | 7.289 | 43.708 | 1.00 | 9.58 |
| ATOM | 423 | NE2 | HIS | 876 | 35.461 | 8.336 | 44.492 | 1.00 | 4.26 |
| ATOM | 424 | C | HIS | 876 | 31.403 | 7.086 | 42.981 | 1.00 | 20.51 |
| ATOM | 425 | O | HIS | 876 | 31.767 | 8.170 | 42.550 | 1.00 | 23.99 |
| ATOM | 426 | N | SER | 877 | 30.770 | 6.265 | 42.170 | 1.00 | 21.46 |
| ATOM | 427 | CA | SER | 877 | 30.609 | 6.552 | 40.723 | 1.00 | 20.65 |
| ATOM | 428 | CB | SER | 877 | 29.710 | 5.476 | 40.128 | 1.00 | 22.58 |
| ATOM | 429 | OG | SER | 877 | 29.710 | 4.315 | 40.979 | 1.00 | 26.51 |
| ATOM | 430 | C | SER | 877 | 30.005 | 7.940 | 40.470 | 1.00 | 20.33 |
| ATOM | 431 | O | SER | 877 | 30.464 | 8.702 | 39.631 | 1.00 | 21.60 |
| ATOM | 432 | N | GLU | 878 | 28.952 | 8.320 | 41.180 | 1.00 | 18.48 |
| ATOM | 433 | CA | GLU | 878 | 28.340 | 9.640 | 41.038 | 1.00 | 15.96 |
| ATOM | 434 | CB | GLU | 878 | 27.093 | 9.798 | 41.803 | 1.00 | 16.55 |
| ATOM | 435 | CG | GLU | 878 | 25.807 | 9.296 | 41.176 | 1.00 | 22.66 |
| ATOM | 436 | CD | GLU | 878 | 25.887 | 7.834 | 40.946 | 1.00 | 22.99 |
| ATOM | 437 | OE1 | GLU | 878 | 26.141 | 7.110 | 41.914 | 1.00 | 22.41 |
| ATOM | 438 | OE2 | GLU | 878 | 25.702 | 7.474 | 39.785 | 1.00 | 26.52 |
| ATOM | 439 | C | GLU | 878 | 29.241 | 10.659 | 41.631 | 1.00 | 16.88 |
| ATOM | 440 | O | GLU | 878 | 29.193 | 11.811 | 41.277 | 1.00 | 23.30 |
| ATOM | 441 | N | HIS | 879 | 29.964 | 10.363 | 42.685 | 1.00 | 15.49 |
| ATOM | 442 | CA | HIS | 879 | 30.897 | 11.354 | 43.264 | 1.00 | 14.40 |
| ATOM | 443 | CB | HIS | 879 | 31.460 | 10.816 | 44.591 | 1.00 | 15.34 |
| ATOM | 444 | CG | HIS | 879 | 32.492 | 11.626 | 45.188 | 1.00 | 9.18 |
| ATOM | 445 | CD2 | HIS | 879 | 32.333 | 12.496 | 46.239 | 1.00 | 8.38 |
| ATOM | 446 | ND1 | HIS | 879 | 33.774 | 11.634 | 44.754 | 1.00 | 8.38 |
| ATOM | 447 | CE1 | HIS | 879 | 34.458 | 12.508 | 45.510 | 1.00 | 8.72 |
| ATOM | 448 | NE2 | HIS | 879 | 33.568 | 13.033 | 46.416 | 1.00 | 12.72 |
| ATOM | 449 | C | HIS | 879 | 31.993 | 11.593 | 42.264 | 1.00 | 16.52 |
| ATOM | 450 | O | HIS | 879 | 32.405 | 12.736 | 42.088 | 1.00 | 21.46 |
| ATOM | 451 | N | ARG | 880 | 32.587 | 10.589 | 41.615 | 1.00 | 16.05 |
| ATOM | 452 | CA | ARG | 880 | 33.564 | 10.992 | 40.624 | 1.00 | 17.19 |
| ATOM | 453 | CB | ARG | 880 | 34.367 | 9.812 | 40.178 | 1.00 | 17.18 |
| ATOM | 454 | CG | ARG | 880 | 33.934 | 8.671 | 39.392 | 1.00 | 24.20 |
| ATOM | 455 | CD | ARG | 880 | 35.133 | 7.671 | 39.502 | 1.00 | 28.49 |
| ATOM | 456 | NE | ARG | 880 | 34.463 | 6.345 | 39.604 | 1.00 | 37.60 |
| ATOM | 457 | CZ | ARG | 880 | 34.812 | 5.366 | 40.451 | 1.00 | 35.68 |
| ATOM | 458 | NH1 | ARG | 880 | 34.117 | 4.255 | 40.441 | 1.00 | 30.70 |
| ATOM | 459 | NH2 | ARG | 880 | 35.873 | 5.433 | 41.251 | 1.00 | 40.55 |
| ATOM | 460 | C | ARG | 880 | 32.867 | 11.698 | 39.458 | 1.00 | 19.11 |
| ATOM | 461 | O | ARG | 880 | 33.422 | 12.665 | 38.954 | 1.00 | 22.50 |
| ATOM | 462 | N | ALA | 881 | 31.672 | 11.323 | 38.977 | 1.00 | 20.35 |
| ATOM | 463 | CA | ALA | 881 | 30.971 | 12.098 | 37.899 | 1.00 | 17.73 |
| ATOM | 464 | CB | ALA | 881 | 29.610 | 11.430 | 37.640 | 1.00 | 12.45 |
| ATOM | 465 | C | ALA | 881 | 30.759 | 13.591 | 38.339 | 1.00 | 17.73 |
| ATOM | 466 | O | ALA | 881 | 30.858 | 14.528 | 37.544 | 1.00 | 20.46 |
| ATOM | 467 | N | LEU | 882 | 30.363 | 13.899 | 39.600 | 1.00 | 18.91 |
| ATOM | 468 | CA | LEU | 882 | 30.232 | 15.284 | 40.142 | 1.00 | 16.53 |
| ATOM | 469 | CB | LEU | 882 | 29.564 | 15.084 | 41.472 | 1.00 | 15.08 |
| ATOM | 470 | CG | LEU | 882 | 29.398 | 16.392 | 42.197 | 1.00 | 15.65 |
| ATOM | 471 | CD1 | LEU | 882 | 28.532 | 17.237 | 41.323 | 1.00 | 18.07 |
| ATOM | 472 | CD2 | LEU | 882 | 28.788 | 16.239 | 43.595 | 1.00 | 10.57 |
| ATOM | 473 | C | LEU | 882 | 31.642 | 16.027 | 40.217 | 1.00 | 18.25 |
| ATOM | 474 | O | LEU | 882 | 31.804 | 17.259 | 40.108 | 1.00 | 17.22 |
| ATOM | 475 | N | MET | 883 | 32.713 | 15.283 | 40.505 | 1.00 | 16.46 |
| ATOM | 476 | CA | MET | 883 | 34.072 | 15.867 | 40.495 | 1.00 | 19.48 |
| ATOM | 477 | CB | MET | 883 | 35.126 | 14.805 | 40.961 | 1.00 | 21.02 |
| ATOM | 478 | CG | MET | 883 | 35.787 | 15.371 | 42.191 | 1.00 | 25.11 |
| ATOM | 479 | SD | MET | 883 | 36.774 | 16.786 | 41.735 | 1.00 | 30.13 |
| ATOM | 480 | CE | MET | 883 | 36.324 | 17.720 | 43.174 | 1.00 | 25.58 |
| ATOM | 481 | C | MET | 883 | 34.352 | 16.283 | 39.040 | 1.00 | 17.59 |
| ATOM | 482 | O | MET | 883 | 34.888 | 17.341 | 38.735 | 1.00 | 20.53 |
| ATOM | 483 | N | SER | 884 | 34.032 | 15.446 | 38.060 | 1.00 | 15.79 |
| ATOM | 484 | CA | SER | 884 | 34.221 | 15.794 | 36.644 | 1.00 | 14.37 |
| ATOM | 485 | CB | SER | 884 | 33.899 | 14.603 | 35.753 | 1.00 | 16.81 |
| ATOM | 486 | OG | SER | 884 | 35.123 | 14.022 | 35.291 | 1.00 | 23.05 |
| ATOM | 487 | C | SER | 884 | 33.332 | 16.975 | 36.198 | 1.00 | 15.23 |
| ATOM | 488 | O | SER | 884 | 33.650 | 17.891 | 35.400 | 1.00 | 15.71 |
| ATOM | 489 | N | GLU | 885 | 32.105 | 16.971 | 36.692 | 1.00 | 12.62 |
| ATOM | 490 | CA | GLU | 885 | 31.259 | 18.088 | 36.335 | 1.00 | 9.20 |
| ATOM | 491 | CB | GLU | 885 | 30.009 | 17.717 | 37.051 | 1.00 | 5.37 |
| ATOM | 492 | CG | GLU | 885 | 29.200 | 18.981 | 36.826 | 1.00 | 11.61 |
| ATOM | 493 | CD | GLU | 885 | 27.801 | 18.839 | 37.258 | 1.00 | 13.93 |
| ATOM | 494 | OE1 | GLU | 885 | 27.351 | 17.748 | 37.536 | 1.00 | 16.89 |
| ATOM | 495 | OE2 | GLU | 885 | 27.174 | 19.848 | 37.318 | 1.00 | 15.79 |
| ATOM | 496 | C | GLU | 885 | 31.943 | 19.458 | 36.723 | 1.00 | 9.01 |
| ATOM | 497 | O | GLU | 885 | 31.895 | 20.487 | 36.053 | 1.00 | 10.45 |
| ATOM | 498 | N | LEU | 886 | 32.596 | 19.507 | 37.848 | 1.00 | 10.65 |
| ATOM | 499 | CA | LEU | 886 | 33.315 | 20.696 | 38.416 | 1.00 | 13.37 |
| ATOM | 500 | CB | LEU | 886 | 33.876 | 20.378 | 39.795 | 1.00 | 8.55 |
| ATOM | 501 | CG | LEU | 886 | 34.819 | 21.374 | 40.443 | 1.00 | 10.16 |
| ATOM | 502 | CD1 | LEU | 886 | 34.097 | 22.640 | 40.770 | 1.00 | 8.44 |
| ATOM | 503 | CD2 | LEU | 886 | 35.363 | 20.769 | 41.761 | 1.00 | 9.44 |
| ATOM | 504 | C | LEU | 886 | 34.439 | 21.034 | 37.544 | 1.00 | 14.43 |
| ATOM | 505 | O | LEU | 886 | 34.738 | 22.178 | 37.216 | 1.00 | 18.34 |
| ATOM | 506 | N | LYS | 887 | 35.143 | 20.011 | 37.129 | 1.00 | 17.06 |
| ATOM | 507 | CA | LYS | 887 | 36.277 | 20.289 | 36.224 | 1.00 | 16.87 |
| ATOM | 508 | CB | LYS | 887 | 37.077 | 18.957 | 36.277 | 1.00 | 14.08 |
| ATOM | 509 | C | LYS | 887 | 35.757 | 20.813 | 34.784 | 1.00 | 18.96 |
| ATOM | 510 | O | LYS | 887 | 36.319 | 21.670 | 34.092 | 1.00 | 20.08 |
| ATOM | 511 | N | ILE | 888 | 34.646 | 20.331 | 34.241 | 1.00 | 19.51 |
| ATOM | 512 | CA | ILE | 888 | 34.078 | 20.821 | 32.972 | 1.00 | 17.20 |
| ATOM | 513 | CB | ILE | 888 | 32.727 | 20.103 | 32.750 | 1.00 | 19.97 |
| ATOM | 514 | CG2 | ILE | 888 | 31.842 | 20.751 | 31.735 | 1.00 | 22.90 |
| ATOM | 515 | CG1 | ILE | 888 | 33.012 | 18.712 | 32.273 | 1.00 | 22.97 |
| ATOM | 516 | CD1 | ILE | 888 | 33.756 | 18.760 | 30.921 | 1.00 | 30.86 |
| ATOM | 517 | C | ILE | 888 | 33.865 | 22.302 | 33.155 | 1.00 | 18.85 |
| ATOM | 518 | O | ILE | 888 | 34.316 | 23.118 | 32.362 | 1.00 | 23.14 |
| ATOM | 519 | N | LEU | 889 | 33.168 | 22.689 | 34.234 | 1.00 | 17.36 |
| ATOM | 520 | CA | LEU | 889 | 32.823 | 24.087 | 34.575 | 1.00 | 10.99 |
| ATOM | 521 | CB | LEU | 889 | 32.084 | 24.146 | 35.943 | 1.00 | 10.17 |
| ATOM | 522 | CG | LEU | 889 | 30.595 | 23.557 | 35.843 | 1.00 | 7.22 |
| ATOM | 523 | CD1 | LEU | 889 | 29.986 | 23.126 | 37.170 | 1.00 | 7.93 |
| ATOM | 524 | CD2 | LEU | 889 | 29.649 | 24.656 | 35.478 | 1.00 | 3.71 |
| ATOM | 525 | C | LEU | 889 | 34.024 | 24.899 | 34.620 | 1.00 | 11.47 |
| ATOM | 526 | O | LEU | 889 | 34.065 | 26.020 | 34.154 | 1.00 | 15.72 |
| ATOM | 527 | N | ILE | 890 | 35.066 | 24.405 | 35.186 | 1.00 | 11.96 |
| ATOM | 528 | CA | ILE | 890 | 36.322 | 25.197 | 35.239 | 1.00 | 13.53 |
| ATOM | 529 | CB | ILE | 890 | 37.359 | 24.530 | 36.270 | 1.00 | 12.08 |
| ATOM | 530 | CG2 | ILE | 890 | 38.627 | 25.345 | 36.206 | 1.00 | 7.44 |
| ATOM | 531 | CG1 | ILE | 890 | 36.851 | 24.438 | 37.724 | 1.00 | 11.21 |
| ATOM | 532 | CD1 | ILE | 890 | 37.843 | 23.417 | 38.377 | 1.00 | 5.47 |
| ATOM | 533 | C | ILE | 890 | 36.943 | 25.227 | 33.834 | 1.00 | 16.10 |
| ATOM | 534 | O | ILE | 890 | 37.569 | 26.192 | 33.397 | 1.00 | 23.32 |
| ATOM | 535 | N | HIS | 891 | 36.878 | 24.155 | 33.086 | 1.00 | 15.97 |
| ATOM | 536 | CA | HIS | 891 | 37.475 | 24.099 | 31.748 | 1.00 | 18.49 |
| ATOM | 537 | CB | HIS | 891 | 37.123 | 22.739 | 31.257 | 1.00 | 25.52 |
| ATOM | 538 | CG | HIS | 891 | 37.403 | 22.327 | 29.883 | 1.00 | 39.08 |
| ATOM | 539 | CD2 | HIS | 891 | 37.748 | 20.965 | 29.657 | 1.00 | 47.43 |
| ATOM | 540 | ND1 | HIS | 891 | 37.332 | 22.999 | 28.706 | 1.00 | 36.50 |
| ATOM | 541 | CE1 | HIS | 891 | 37.646 | 22.052 | 27.750 | 1.00 | 45.68 |
| ATOM | 542 | NE2 | HIS | 891 | 37.903 | 20.804 | 28.316 | 1.00 | 52.82 |
| ATOM | 543 | C | HIS | 891 | 36.872 | 25.215 | 30.901 | 1.00 | 18.99 |
| ATOM | 544 | O | HIS | 891 | 37.592 | 25.918 | 30.242 | 1.00 | 19.59 |
| ATOM | 545 | N | ILE | 892 | 35.540 | 25.332 | 30.923 | 1.00 | 16.55 |
| ATOM | 546 | CA | ILE | 892 | 34.670 | 26.271 | 30.200 | 1.00 | 16.57 |
| ATOM | 547 | CB | ILE | 892 | 33.147 | 25.834 | 30.355 | 1.00 | 17.81 |
| ATOM | 548 | CG2 | ILE | 892 | 32.113 | 26.560 | 29.475 | 1.00 | 14.77 |
| ATOM | 549 | CG1 | ILE | 892 | 33.039 | 24.439 | 29.818 | 1.00 | 17.20 |
| ATOM | 550 | CD1 | ILE | 892 | 31.684 | 23.866 | 30.202 | 1.00 | 18.02 |
| ATOM | 551 | C | ILE | 892 | 34.825 | 27.706 | 30.611 | 1.00 | 19.27 |
| ATOM | 552 | O | ILE | 892 | 34.742 | 28.584 | 29.783 | 1.00 | 23.96 |
| ATOM | 553 | N | GLY | 893 | 35.116 | 28.032 | 31.853 | 1.00 | 19.14 |
| ATOM | 554 | CA | GLY | 893 | 35.224 | 29.431 | 32.202 | 1.00 | 17.73 |
| ATOM | 555 | C | GLY | 893 | 33.896 | 30.158 | 32.137 | 1.00 | 18.74 |
| ATOM | 556 | O | GLY | 893 | 32.828 | 29.696 | 31.770 | 1.00 | 21.86 |
| ATOM | 557 | N | HIS | 894 | 33.973 | 31.390 | 32.523 | 1.00 | 19.51 |
| ATOM | 558 | CA | HIS | 894 | 32.842 | 32.271 | 32.585 | 1.00 | 19.67 |
| ATOM | 559 | CB | HIS | 894 | 33.369 | 33.385 | 33.468 | 1.00 | 22.88 |
| ATOM | 560 | CG | HIS | 894 | 32.281 | 34.319 | 33.797 | 1.00 | 31.11 |
| ATOM | 561 | CD2 | HIS | 894 | 31.205 | 34.062 | 34.675 | 1.00 | 34.22 |
| ATOM | 562 | ND1 | HIS | 894 | 32.082 | 35.570 | 33.249 | 1.00 | 33.44 |
| ATOM | 563 | CE1 | HIS | 894 | 30.911 | 36.083 | 33.772 | 1.00 | 34.31 |
| ATOM | 564 | NE2 | HIS | 894 | 30.371 | 35.156 | 34.647 | 1.00 | 37.05 |
| ATOM | 565 | C | HIS | 894 | 32.125 | 32.782 | 31.353 | 1.00 | 18.43 |
| ATOM | 566 | O | HIS | 894 | 32.730 | 33.206 | 30.371 | 1.00 | 18.52 |
| ATOM | 567 | N | HIS | 895 | 30.801 | 32.955 | 31.480 | 1.00 | 14.90 |
| ATOM | 568 | CA | HIS | 895 | 30.050 | 33.531 | 30.380 | 1.00 | 14.12 |
| ATOM | 569 | CB | HIS | 895 | 29.814 | 32.537 | 29.223 | 1.00 | 13.87 |
| ATOM | 570 | CG | HIS | 895 | 29.106 | 33.224 | 28.095 | 1.00 | 8.87 |
| ATOM | 571 | CD2 | HIS | 895 | 29.677 | 33.876 | 27.019 | 1.00 | 8.51 |
| ATOM | 572 | ND1 | HIS | 895 | 27.763 | 33.439 | 28.022 | 1.00 | 11.01 |
| ATOM | 573 | CE1 | HIS | 895 | 27.529 | 34.214 | 26.922 | 1.00 | 9.89 |
| ATOM | 574 | NE2 | HIS | 895 | 28.709 | 34.481 | 26.304 | 1.00 | 6.18 |
| ATOM | 575 | C | HIS | 895 | 28.717 | 34.007 | 30.849 | 1.00 | 16.62 |
| ATOM | 576 | O | HIS | 895 | 28.026 | 33.338 | 31.586 | 1.00 | 20.68 |
| ATOM | 577 | N | LEU | 896 | 28.195 | 35.144 | 30.362 | 1.00 | 18.72 |
| ATOM | 578 | CA | LEU | 896 | 26.885 | 35.591 | 30.884 | 1.00 | 15.99 |
| ATOM | 579 | CB | LEU | 896 | 26.412 | 36.861 | 30.083 | 1.00 | 16.94 |
| ATOM | 580 | CG | LEU | 896 | 25.230 | 37.618 | 30.779 | 1.00 | 18.50 |
| ATOM | 581 | CD1 | LEU | 896 | 25.790 | 38.265 | 32.059 | 1.00 | 19.27 |
| ATOM | 582 | CD2 | LEU | 896 | 24.580 | 38.678 | 29.871 | 1.00 | 19.06 |
| ATOM | 583 | C | LEU | 896 | 25.799 | 34.511 | 30.832 | 1.00 | 15.34 |
| ATOM | 584 | O | LEU | 896 | 25.032 | 34.257 | 31.744 | 1.00 | 14.29 |
| ATOM | 585 | N | ASN | 897 | 25.748 | 33.872 | 29.694 | 1.00 | 15.82 |
| ATOM | 586 | CA | ASN | 897 | 24.666 | 32.901 | 29.496 | 1.00 | 14.54 |
| ATOM | 587 | CB | ASN | 897 | 24.192 | 33.005 | 28.013 | 1.00 | 14.37 |
| ATOM | 588 | CG | ASN | 897 | 23.769 | 34.473 | 27.680 | 1.00 | 14.23 |
| ATOM | 589 | OD1 | ASN | 897 | 24.437 | 35.216 | 26.980 | 1.00 | 14.52 |
| ATOM | 590 | ND2 | ASN | 897 | 22.779 | 35.059 | 28.271 | 1.00 | 12.07 |
| ATOM | 591 | C | ASN | 897 | 25.000 | 31.531 | 29.888 | 1.00 | 15.84 |
| ATOM | 592 | O | ASN | 897 | 24.353 | 30.649 | 29.378 | 1.00 | 17.61 |
| ATOM | 593 | N | VAL | 898 | 26.083 | 31.259 | 30.592 | 1.00 | 16.01 |
| ATOM | 594 | CA | VAL | 898 | 26.202 | 29.827 | 31.067 | 1.00 | 16.40 |
| ATOM | 595 | CB | VAL | 898 | 27.545 | 29.024 | 30.579 | 1.00 | 10.61 |
| ATOM | 596 | CG1 | VAL | 898 | 27.772 | 29.204 | 29.098 | 1.00 | 5.98 |
| ATOM | 597 | CG2 | VAL | 898 | 28.801 | 29.517 | 31.173 | 1.00 | 16.22 |
| ATOM | 598 | C | VAL | 898 | 26.153 | 29.965 | 32.643 | 1.00 | 20.93 |
| ATOM | 599 | O | VAL | 898 | 26.729 | 30.938 | 33.180 | 1.00 | 21.50 |
| ATOM | 600 | N | VAL | 899 | 25.360 | 29.178 | 33.437 | 1.00 | 22.66 |
| ATOM | 601 | CA | VAL | 899 | 25.352 | 29.362 | 34.935 | 1.00 | 17.23 |
| ATOM | 602 | CB | VAL | 899 | 24.317 | 28.411 | 35.625 | 1.00 | 17.12 |
| ATOM | 603 | CG1 | VAL | 899 | 24.739 | 26.943 | 35.593 | 1.00 | 13.70 |
| ATOM | 604 | CG2 | VAL | 899 | 24.162 | 28.846 | 37.058 | 1.00 | 12.86 |
| ATOM | 605 | C | VAL | 899 | 26.733 | 29.112 | 35.522 | 1.00 | 19.01 |
| ATOM | 606 | O | VAL | 899 | 27.276 | 28.022 | 35.618 | 1.00 | 20.49 |
| ATOM | 607 | N | ASN | 900 | 27.362 | 30.188 | 35.878 | 1.00 | 19.93 |
| ATOM | 608 | CA | ASN | 900 | 28.702 | 30.103 | 36.425 | 1.00 | 20.69 |
| ATOM | 609 | CB | ASN | 900 | 29.364 | 31.448 | 36.047 | 1.00 | 28.34 |
| ATOM | 610 | CG | ASN | 900 | 29.953 | 31.091 | 34.658 | 1.00 | 32.05 |
| ATOM | 611 | OD1 | ASN | 900 | 31.026 | 30.542 | 34.456 | 1.00 | 33.74 |
| ATOM | 612 | ND2 | ASN | 900 | 29.265 | 31.355 | 33.601 | 1.00 | 30.58 |
| ATOM | 613 | C | ASN | 900 | 29.048 | 29.715 | 37.833 | 1.00 | 16.09 |
| ATOM | 614 | O | ASN | 900 | 28.393 | 30.014 | 38.825 | 1.00 | 12.14 |
| ATOM | 615 | N | LEU | 901 | 30.201 | 29.048 | 37.823 | 1.00 | 14.82 |
| ATOM | 616 | CA | LEU | 901 | 30.885 | 28.515 | 38.994 | 1.00 | 13.72 |
| ATOM | 617 | CB | LEU | 901 | 31.958 | 27.497 | 38.492 | 1.00 | 6.02 |
| ATOM | 618 | CG | LEU | 901 | 32.649 | 26.658 | 39.598 | 1.00 | 2.00 |
| ATOM | 619 | CD1 | LEU | 901 | 31.640 | 25.731 | 40.189 | 1.00 | 2.00 |
| ATOM | 620 | CD2 | LEU | 901 | 33.843 | 25.912 | 39.047 | 1.00 | 2.08 |
| ATOM | 621 | C | LEU | 901 | 31.508 | 29.569 | 39.923 | 1.00 | 14.47 |
| ATOM | 622 | O | LEU | 901 | 32.429 | 30.300 | 39.519 | 1.00 | 17.14 |
| ATOM | 623 | N | LEU | 902 | 31.027 | 29.556 | 41.192 | 1.00 | 12.25 |
| ATOM | 624 | CA | LEU | 902 | 31.439 | 30.462 | 42.278 | 1.00 | 15.05 |
| ATOM | 625 | CB | LEU | 902 | 30.184 | 30.788 | 43.123 | 1.00 | 11.81 |
| ATOM | 626 | CG | LEU | 902 | 29.215 | 31.794 | 42.443 | 1.00 | 9.38 |
| ATOM | 627 | CD1 | LEU | 902 | 27.875 | 31.868 | 43.184 | 1.00 | 4.41 |
| ATOM | 628 | CD2 | LEU | 902 | 29.964 | 33.130 | 42.342 | 1.00 | 8.98 |
| ATOM | 629 | C | LEU | 902 | 32.566 | 30.026 | 43.208 | 1.00 | 18.79 |
| ATOM | 630 | O | LEU | 902 | 33.431 | 30.791 | 43.663 | 1.00 | 21.69 |
| ATOM | 631 | N | GLY | 903 | 32.501 | 28.746 | 43.547 | 1.00 | 20.37 |
| ATOM | 632 | CA | GLY | 903 | 33.493 | 28.087 | 44.428 | 1.00 | 21.53 |
| ATOM | 633 | C | GLY | 903 | 33.201 | 26.614 | 44.464 | 1.00 | 19.61 |
| ATOM | 634 | O | GLY | 903 | 32.335 | 26.096 | 43.756 | 1.00 | 23.64 |
| ATOM | 635 | N | ALA | 904 | 33.855 | 25.899 | 45.340 | 1.00 | 21.35 |
| ATOM | 636 | CA | ALA | 904 | 33.670 | 24.419 | 45.481 | 1.00 | 18.91 |
| ATOM | 637 | CB | ALA | 904 | 34.474 | 23.798 | 44.382 | 1.00 | 12.92 |
| ATOM | 638 | C | ALA | 904 | 34.090 | 23.778 | 46.832 | 1.00 | 18.78 |
| ATOM | 639 | O | ALA | 904 | 35.148 | 24.053 | 47.384 | 1.00 | 19.77 |
| ATOM | 640 | N | CYS | 905 | 33.275 | 23.014 | 47.503 | 1.00 | 19.99 |
| ATOM | 641 | CA | CYS | 905 | 33.732 | 22.336 | 48.730 | 1.00 | 22.16 |
| ATOM | 642 | CB | CYS | 905 | 32.643 | 22.224 | 49.733 | 1.00 | 23.70 |
| ATOM | 643 | SG | CYS | 905 | 32.175 | 23.897 | 49.985 | 1.00 | 27.92 |
| ATOM | 644 | C | CYS | 905 | 34.193 | 20.906 | 48.366 | 1.00 | 26.00 |
| ATOM | 645 | O | CYS | 905 | 33.404 | 20.010 | 48.071 | 1.00 | 25.09 |
| ATOM | 646 | N | THR | 906 | 35.514 | 20.752 | 48.223 | 1.00 | 29.25 |
| ATOM | 647 | CA | THR | 906 | 36.318 | 19.528 | 47.881 | 1.00 | 32.05 |
| ATOM | 648 | CB | THR | 906 | 37.267 | 19.955 | 46.687 | 1.00 | 32.21 |
| ATOM | 649 | OG1 | THR | 906 | 36.495 | 19.773 | 45.503 | 1.00 | 34.54 |
| ATOM | 650 | CG2 | THR | 906 | 38.634 | 19.263 | 46.598 | 1.00 | 31.99 |
| ATOM | 651 | C | THR | 906 | 37.118 | 18.929 | 49.059 | 1.00 | 34.88 |
| ATOM | 652 | O | THR | 906 | 37.675 | 17.850 | 48.928 | 1.00 | 35.78 |
| ATOM | 653 | N | LYS | 907 | 37.224 | 19.526 | 50.253 | 1.00 | 38.01 |
| ATOM | 654 | CA | LYS | 907 | 38.058 | 18.885 | 51.326 | 1.00 | 41.35 |
| ATOM | 655 | CB | LYS | 907 | 38.201 | 19.814 | 52.622 | 1.00 | 43.73 |
| ATOM | 656 | CG | LYS | 907 | 39.156 | 21.062 | 52.484 | 1.00 | 48.01 |
| ATOM | 657 | CD | LYS | 907 | 39.205 | 22.213 | 53.600 | 1.00 | 56.47 |
| ATOM | 658 | CE | LYS | 907 | 38.325 | 23.584 | 53.660 | 1.00 | 57.48 |
| ATOM | 659 | NZ | LYS | 907 | 36.981 | 23.498 | 54.317 | 1.00 | 53.54 |
| ATOM | 660 | C | LYS | 907 | 37.557 | 17.503 | 51.800 | 1.00 | 40.98 |
| ATOM | 661 | O | LYS | 907 | 36.395 | 17.382 | 52.162 | 1.00 | 41.98 |
| ATOM | 662 | N | PRO | 908 | 38.403 | 16.445 | 51.852 | 1.00 | 41.38 |
| ATOM | 663 | CD | PRO | 908 | 39.750 | 16.379 | 51.252 | 1.00 | 42.56 |
| ATOM | 664 | CA | PRO | 908 | 38.154 | 15.123 | 52.428 | 1.00 | 40.42 |
| ATOM | 665 | CB | PRO | 908 | 39.532 | 14.761 | 52.976 | 1.00 | 41.35 |
| ATOM | 666 | CG | PRO | 908 | 40.325 | 15.044 | 51.728 | 1.00 | 42.20 |
| ATOM | 667 | C | PRO | 908 | 37.038 | 14.851 | 53.434 | 1.00 | 39.77 |
| ATOM | 668 | O | PRO | 908 | 36.174 | 13.975 | 53.214 | 1.00 | 39.42 |
| ATOM | 669 | N | GLY | 909 | 37.099 | 15.548 | 54.562 | 1.00 | 37.56 |
| ATOM | 670 | CA | GLY | 909 | 36.102 | 15.344 | 55.642 | 1.00 | 39.08 |
| ATOM | 671 | C | GLY | 909 | 34.598 | 15.328 | 55.297 | 1.00 | 40.37 |
| ATOM | 672 | O | GLY | 909 | 33.779 | 15.008 | 56.171 | 1.00 | 40.25 |
| ATOM | 673 | N | GLY | 910 | 34.226 | 15.652 | 54.039 | 1.00 | 39.29 |
| ATOM | 674 | CA | GLY | 910 | 32.819 | 15.700 | 53.602 | 1.00 | 35.87 |
| ATOM | 675 | C | GLY | 910 | 32.498 | 15.590 | 52.084 | 1.00 | 35.30 |
| ATOM | 676 | O | GLY | 910 | 33.353 | 15.191 | 51.258 | 1.00 | 35.48 |
| ATOM | 677 | N | PRO | 911 | 31.214 | 15.962 | 51.745 | 1.00 | 33.68 |
| ATOM | 678 | CD | PRO | 911 | 30.266 | 16.483 | 52.764 | 1.00 | 30.51 |
| ATOM | 679 | CA | PRO | 911 | 30.462 | 15.844 | 50.452 | 1.00 | 29.99 |
| ATOM | 680 | CB | PRO | 911 | 29.087 | 16.473 | 50.736 | 1.00 | 29.87 |
| ATOM | 681 | CG | PRO | 911 | 28.902 | 16.158 | 52.179 | 1.00 | 30.21 |
| ATOM | 682 | C | PRO | 911 | 30.860 | 16.290 | 49.024 | 1.00 | 28.55 |
| ATOM | 683 | O | PRO | 911 | 30.343 | 15.710 | 48.092 | 1.00 | 29.45 |
| ATOM | 684 | N | LEU | 912 | 31.775 | 17.164 | 48.682 | 1.00 | 24.28 |
| ATOM | 685 | CA | LEU | 912 | 31.926 | 17.583 | 47.251 | 1.00 | 21.33 |
| ATOM | 686 | CB | LEU | 912 | 32.247 | 16.405 | 46.239 | 1.00 | 18.13 |
| ATOM | 687 | CG | LEU | 912 | 32.249 | 16.888 | 44.761 | 1.00 | 15.15 |
| ATOM | 688 | CD1 | LEU | 912 | 33.297 | 17.944 | 44.590 | 1.00 | 10.60 |
| ATOM | 689 | CD2 | LEU | 912 | 32.616 | 15.782 | 43.784 | 1.00 | 14.65 |
| ATOM | 690 | C | LEU | 912 | 30.592 | 18.300 | 46.835 | 1.00 | 19.91 |
| ATOM | 691 | O | LEU | 912 | 29.446 | 17.827 | 46.688 | 1.00 | 19.47 |
| ATOM | 692 | N | MET | 913 | 30.781 | 19.618 | 46.909 | 1.00 | 17.86 |
| ATOM | 693 | CA | MET | 913 | 29.734 | 20.577 | 46.604 | 1.00 | 12.54 |
| ATOM | 694 | CB | MET | 913 | 29.500 | 21.414 | 47.866 | 1.00 | 13.81 |
| ATOM | 695 | CG | MET | 913 | 28.938 | 20.461 | 48.945 | 1.00 | 15.32 |
| ATOM | 696 | SD | MET | 913 | 28.524 | 21.381 | 50.381 | 1.00 | 20.78 |
| ATOM | 697 | CE | MET | 913 | 26.977 | 20.631 | 50.725 | 1.00 | 7.67 |
| ATOM | 698 | C | MET | 913 | 30.222 | 21.413 | 45.482 | 1.00 | 14.29 |
| ATOM | 699 | O | MET | 913 | 31.399 | 21.806 | 45.509 | 1.00 | 11.82 |
| ATOM | 700 | N | VAL | 914 | 29.404 | 21.562 | 44.409 | 1.00 | 15.66 |
| ATOM | 701 | CA | VAL | 914 | 29.742 | 22.454 | 43.224 | 1.00 | 11.17 |
| ATOM | 702 | CB | VAL | 914 | 29.502 | 21.830 | 41.861 | 1.00 | 10.37 |
| ATOM | 703 | CG1 | VAL | 914 | 29.970 | 22.826 | 40.775 | 1.00 | 4.14 |
| ATOM | 704 | CG2 | VAL | 914 | 30.267 | 20.505 | 41.746 | 1.00 | 6.36 |
| ATOM | 705 | C | VAL | 914 | 28.742 | 23.584 | 43.386 | 1.00 | 11.54 |
| ATOM | 706 | O | VAL | 914 | 27.507 | 23.385 | 43.384 | 1.00 | 8.16 |
| ATOM | 707 | N | ILE | 915 | 29.330 | 24.748 | 43.770 | 1.00 | 12.95 |
| ATOM | 708 | CA | ILE | 915 | 28.569 | 26.020 | 44.085 | 1.00 | 10.65 |
| ATOM | 709 | CB | ILE | 915 | 29.259 | 26.740 | 45.250 | 1.00 | 6.19 |
| ATOM | 710 | CG2 | ILE | 915 | 28.386 | 27.841 | 45.762 | 1.00 | 5.76 |
| ATOM | 711 | CG1 | ILE | 915 | 29.510 | 25.744 | 46.361 | 1.00 | 6.42 |
| ATOM | 712 | CD1 | ILE | 915 | 30.256 | 26.243 | 47.575 | 1.00 | 4.34 |
| ATOM | 713 | C | ILE | 915 | 28.420 | 26.997 | 42.938 | 1.00 | 11.96 |
| ATOM | 714 | O | ILE | 915 | 29.436 | 27.474 | 42.419 | 1.00 | 14.70 |
| ATOM | 715 | N | VAL | 916 | 27.210 | 27.291 | 42.507 | 1.00 | 9.86 |
| ATOM | 716 | CA | VAL | 916 | 27.110 | 28.234 | 41.428 | 1.00 | 8.13 |
| ATOM | 717 | CB | VAL | 916 | 26.659 | 27.517 | 40.138 | 1.00 | 8.09 |
| ATOM | 718 | CG1 | VAL | 916 | 27.584 | 26.355 | 39.687 | 1.00 | 4.58 |
| ATOM | 719 | CG2 | VAL | 916 | 25.272 | 27.040 | 40.398 | 1.00 | 7.77 |
| ATOM | 720 | C | VAL | 916 | 26.115 | 29.334 | 41.828 | 1.00 | 13.91 |
| ATOM | 721 | O | VAL | 916 | 25.484 | 29.315 | 42.903 | 1.00 | 12.37 |
| ATOM | 722 | N | GLU | 917 | 26.011 | 30.346 | 40.950 | 1.00 | 16.12 |
| ATOM | 723 | CA | GLU | 917 | 25.094 | 31.480 | 41.201 | 1.00 | 13.77 |
| ATOM | 724 | CB | GLU | 917 | 25.169 | 32.518 | 40.096 | 1.00 | 15.29 |
| ATOM | 725 | CG | GLU | 917 | 24.622 | 32.071 | 38.758 | 1.00 | 19.03 |
| ATOM | 726 | CD | GLU | 917 | 24.903 | 33.174 | 37.754 | 1.00 | 23.36 |
| ATOM | 727 | OE1 | GLU | 917 | 26.018 | 33.290 | 37.181 | 1.00 | 29.66 |
| ATOM | 728 | OE2 | GLU | 917 | 23.963 | 33.938 | 37.580 | 1.00 | 24.20 |
| ATOM | 729 | C | GLU | 917 | 23.718 | 31.035 | 41.280 | 1.00 | 12.19 |
| ATOM | 730 | O | GLU | 917 | 23.383 | 29.916 | 40.891 | 1.00 | 14.59 |
| ATOM | 731 | N | PHE | 918 | 22.943 | 31.828 | 41.940 | 1.00 | 10.59 |
| ATOM | 732 | CA | PHE | 918 | 21.537 | 31.507 | 42.045 | 1.00 | 10.95 |
| ATOM | 733 | CB | PHE | 918 | 21.136 | 31.874 | 43.389 | 1.00 | 11.29 |
| ATOM | 734 | CG | PHE | 918 | 19.716 | 31.681 | 43.644 | 1.00 | 9.81 |
| ATOM | 735 | CD1 | PHE | 918 | 19.233 | 30.413 | 43.803 | 1.00 | 4.01 |
| ATOM | 736 | CD2 | PHE | 918 | 18.911 | 32.802 | 43.846 | 1.00 | 9.25 |
| ATOM | 737 | CE1 | PHE | 918 | 17.921 | 30.274 | 44.200 | 1.00 | 8.95 |
| ATOM | 738 | CE2 | PHE | 918 | 17.589 | 32.641 | 44.238 | 1.00 | 12.65 |
| ATOM | 739 | CZ | PHE | 918 | 17.092 | 31.369 | 44.421 | 1.00 | 7.31 |
| ATOM | 740 | C | PHE | 918 | 20.859 | 32.337 | 40.988 | 1.00 | 13.15 |
| ATOM | 741 | O | PHE | 918 | 21.228 | 33.472 | 40.742 | 1.00 | 20.08 |
| ATOM | 742 | N | CYS | 919 | 19.781 | 31.858 | 40.408 | 1.00 | 15.35 |
| ATOM | 743 | CA | CYS | 919 | 19.005 | 32.483 | 39.341 | 1.00 | 13.38 |
| ATOM | 744 | CB | CYS | 919 | 19.091 | 31.622 | 38.108 | 1.00 | 11.01 |
| ATOM | 745 | SG | CYS | 919 | 20.815 | 31.595 | 37.566 | 1.00 | 13.57 |
| ATOM | 746 | C | CYS | 919 | 17.605 | 32.561 | 39.846 | 1.00 | 15.20 |
| ATOM | 747 | O | CYS | 919 | 16.855 | 31.588 | 39.841 | 1.00 | 16.30 |
| ATOM | 748 | N | LYS | 920 | 17.236 | 33.790 | 40.221 | 1.00 | 17.10 |
| ATOM | 749 | CA | LYS | 920 | 15.921 | 34.091 | 40.819 | 1.00 | 17.53 |
| ATOM | 750 | CB | LYS | 920 | 15.724 | 35.633 | 40.698 | 1.00 | 24.74 |
| ATOM | 751 | CG | LYS | 920 | 15.006 | 36.486 | 41.828 | 1.00 | 34.84 |
| ATOM | 752 | CD | LYS | 920 | 15.634 | 36.439 | 43.312 | 1.00 | 45.35 |
| ATOM | 753 | CE | LYS | 920 | 17.155 | 36.898 | 43.481 | 1.00 | 50.81 |
| ATOM | 754 | NZ | LYS | 920 | 17.691 | 36.715 | 44.857 | 1.00 | 54.90 |
| ATOM | 755 | C | LYS | 920 | 14.695 | 33.357 | 40.309 | 1.00 | 15.34 |
| ATOM | 756 | O | LYS | 920 | 13.969 | 32.710 | 41.066 | 1.00 | 14.09 |
| ATOM | 757 | N | PHE | 921 | 14.431 | 33.354 | 39.012 | 1.00 | 16.41 |
| ATOM | 758 | CA | PHE | 921 | 13.162 | 32.702 | 38.591 | 1.00 | 16.42 |
| ATOM | 759 | CB | PHE | 921 | 12.623 | 33.474 | 37.407 | 1.00 | 15.56 |
| ATOM | 760 | CG | PHE | 921 | 12.381 | 34.875 | 37.715 | 1.00 | 11.43 |
| ATOM | 761 | CD1 | PHE | 921 | 11.133 | 35.301 | 38.188 | 1.00 | 13.96 |
| ATOM | 762 | CD2 | PHE | 921 | 13.409 | 35.789 | 37.490 | 1.00 | 10.80 |
| ATOM | 763 | CE1 | PHE | 921 | 10.931 | 36.669 | 38.421 | 1.00 | 10.85 |
| ATOM | 764 | CE2 | PHE | 921 | 13.230 | 37.144 | 37.722 | 1.00 | 7.88 |
| ATOM | 765 | CZ | PHE | 921 | 11.982 | 37.556 | 38.182 | 1.00 | 9.57 |
| ATOM | 766 | C | PHE | 921 | 13.140 | 31.223 | 38.266 | 1.00 | 16.32 |
| ATOM | 767 | O | PHE | 921 | 12.165 | 30.741 | 37.674 | 1.00 | 16.67 |
| ATOM | 768 | N | GLY | 922 | 14.168 | 30.477 | 38.651 | 1.00 | 16.52 |
| ATOM | 769 | CA | GLY | 922 | 14.168 | 29.028 | 38.346 | 1.00 | 17.83 |
| ATOM | 770 | C | GLY | 922 | 14.324 | 28.705 | 36.867 | 1.00 | 17.56 |
| ATOM | 771 | O | GLY | 922 | 14.820 | 29.490 | 36.066 | 1.00 | 19.39 |
| ATOM | 772 | N | ASN | 923 | 14.014 | 27.489 | 36.450 | 1.00 | 17.87 |
| ATOM | 773 | CA | ASN | 923 | 14.117 | 27.141 | 35.005 | 1.00 | 17.70 |
| ATOM | 774 | CB | ASN | 923 | 13.931 | 25.592 | 34.812 | 1.00 | 21.51 |
| ATOM | 775 | CG | ASN | 923 | 12.501 | 25.170 | 34.961 | 1.00 | 23.57 |
| ATOM | 776 | OD1 | ASN | 923 | 11.840 | 24.781 | 34.002 | 1.00 | 28.51 |
| ATOM | 777 | ND2 | ASN | 923 | 11.909 | 25.303 | 36.139 | 1.00 | 30.28 |
| ATOM | 778 | C | ASN | 923 | 13.133 | 27.899 | 34.076 | 1.00 | 15.60 |
| ATOM | 779 | O | ASN | 923 | 11.974 | 28.189 | 34.422 | 1.00 | 15.07 |
| ATOM | 780 | N | LEU | 924 | 13.505 | 28.075 | 32.829 | 1.00 | 9.86 |
| ATOM | 781 | CA | LEU | 924 | 12.655 | 28.856 | 31.934 | 1.00 | 14.93 |
| ATOM | 782 | CB | LEU | 924 | 13.603 | 29.259 | 30.756 | 1.00 | 14.02 |
| ATOM | 783 | CG | LEU | 924 | 13.142 | 30.325 | 29.736 | 1.00 | 9.23 |
| ATOM | 784 | CD1 | LEU | 924 | 12.903 | 31.713 | 30.328 | 1.00 | 5.52 |
| ATOM | 785 | CD2 | LEU | 924 | 14.278 | 30.375 | 28.724 | 1.00 | 8.33 |
| ATOM | 786 | C | LEU | 924 | 11.322 | 28.218 | 31.489 | 1.00 | 17.10 |
| ATOM | 787 | O | LEU | 924 | 10.310 | 28.867 | 31.240 | 1.00 | 16.34 |
| ATOM | 788 | N | SER | 925 | 11.314 | 26.904 | 31.421 | 1.00 | 19.35 |
| ATOM | 789 | CA | SER | 925 | 10.137 | 26.081 | 31.011 | 1.00 | 20.31 |
| ATOM | 790 | CB | SER | 925 | 10.588 | 24.598 | 31.241 | 1.00 | 21.42 |
| ATOM | 791 | OG | SER | 925 | 9.633 | 23.628 | 30.799 | 1.00 | 20.73 |
| ATOM | 792 | C | SER | 925 | 8.957 | 26.514 | 31.859 | 1.00 | 17.91 |
| ATOM | 793 | O | SER | 925 | 8.102 | 27.311 | 31.508 | 1.00 | 20.23 |
| ATOM | 794 | N | THR | 926 | 9.065 | 26.135 | 33.101 | 1.00 | 15.92 |
| ATOM | 795 | CA | THR | 926 | 8.120 | 26.431 | 34.180 | 1.00 | 13.45 |
| ATOM | 796 | CB | THR | 926 | 8.743 | 25.960 | 35.573 | 1.00 | 14.56 |
| ATOM | 797 | OG1 | THR | 926 | 9.044 | 24.584 | 35.443 | 1.00 | 16.18 |
| ATOM | 798 | CG2 | THR | 926 | 7.826 | 26.099 | 36.817 | 1.00 | 17.03 |
| ATOM | 799 | C | THR | 926 | 7.886 | 27.921 | 34.210 | 1.00 | 11.53 |
| ATOM | 800 | O | THR | 926 | 6.769 | 28.326 | 34.470 | 1.00 | 14.49 |
| ATOM | 801 | N | TYR | 927 | 8.901 | 28.750 | 33.996 | 1.00 | 12.04 |
| ATOM | 802 | CA | TYR | 927 | 8.706 | 30.220 | 34.051 | 1.00 | 14.59 |
| ATOM | 803 | CB | TYR | 927 | 10.044 | 31.024 | 33.886 | 1.00 | 18.02 |
| ATOM | 804 | CG | TYR | 927 | 9.816 | 32.493 | 33.942 | 1.00 | 17.50 |
| ATOM | 805 | CD1 | TYR | 927 | 9.521 | 33.125 | 35.143 | 1.00 | 19.43 |
| ATOM | 806 | CE1 | TYR | 927 | 9.246 | 34.505 | 35.166 | 1.00 | 16.89 |
| ATOM | 807 | CD2 | TYR | 927 | 9.841 | 33.262 | 32.778 | 1.00 | 18.83 |
| ATOM | 808 | CE2 | TYR | 927 | 9.567 | 34.648 | 32.814 | 1.00 | 19.37 |
| ATOM | 809 | CZ | TYR | 927 | 9.259 | 35.283 | 34.009 | 1.00 | 13.96 |
| ATOM | 810 | OH | TYR | 927 | 8.862 | 36.619 | 34.041 | 1.00 | 15.28 |
| ATOM | 811 | C | TYR | 927 | 7.790 | 30.625 | 32.958 | 1.00 | 12.39 |
| ATOM | 812 | O | TYR | 927 | 6.791 | 31.270 | 33.247 | 1.00 | 14.14 |
| ATOM | 813 | N | LEU | 928 | 8.046 | 30.233 | 31.699 | 1.00 | 10.10 |
| ATOM | 814 | CA | LEU | 928 | 7.110 | 30.634 | 30.609 | 1.00 | 10.25 |
| ATOM | 815 | CB | LEU | 928 | 7.738 | 30.164 | 29.267 | 1.00 | 7.34 |
| ATOM | 816 | CG | LEU | 928 | 9.009 | 31.000 | 29.029 | 1.00 | 6.92 |
| ATOM | 817 | CD1 | LEU | 928 | 9.777 | 30.451 | 27.844 | 1.00 | 2.00 |
| ATOM | 818 | CD2 | LEU | 928 | 8.648 | 32.477 | 28.947 | 1.00 | 2.64 |
| ATOM | 819 | C | LEU | 928 | 5.639 | 30.122 | 30.809 | 1.00 | 12.50 |
| ATOM | 820 | O | LEU | 928 | 4.710 | 30.945 | 30.807 | 1.00 | 12.33 |
| ATOM | 821 | N | ARG | 929 | 5.313 | 28.830 | 31.140 | 1.00 | 14.38 |
| ATOM | 822 | CA | ARG | 929 | 3.827 | 28.493 | 31.282 | 1.00 | 17.18 |
| ATOM | 823 | CB | ARG | 929 | 3.486 | 26.957 | 31.429 | 1.00 | 12.94 |
| ATOM | 824 | CG | ARG | 929 | 4.696 | 26.159 | 31.537 | 1.00 | 19.00 |
| ATOM | 825 | CD | ARG | 929 | 4.358 | 24.714 | 31.794 | 1.00 | 23.47 |
| ATOM | 826 | NE | ARG | 929 | 5.476 | 24.261 | 32.690 | 1.00 | 32.26 |
| ATOM | 827 | CZ | ARG | 929 | 6.548 | 23.547 | 32.254 | 1.00 | 37.30 |
| ATOM | 828 | NH1 | ARG | 929 | 7.508 | 23.210 | 33.140 | 1.00 | 39.58 |
| ATOM | 829 | NH2 | ARG | 929 | 6.634 | 23.103 | 30.968 | 1.00 | 39.60 |
| ATOM | 830 | C | ARG | 929 | 3.085 | 29.189 | 32.413 | 1.00 | 18.46 |
| ATOM | 831 | O | ARG | 929 | 1.859 | 29.103 | 32.633 | 1.00 | 22.68 |
| ATOM | 832 | N | SER | 930 | 3.825 | 29.950 | 33.167 | 1.00 | 19.35 |
| ATOM | 833 | CA | SER | 930 | 3.162 | 30.737 | 34.237 | 1.00 | 20.51 |
| ATOM | 834 | CB | SER | 930 | 4.127 | 30.837 | 35.465 | 1.00 | 21.35 |
| ATOM | 835 | OG | SER | 930 | 5.277 | 31.600 | 35.191 | 1.00 | 19.98 |
| ATOM | 836 | C | SER | 930 | 2.771 | 32.115 | 33.712 | 1.00 | 17.66 |
| ATOM | 837 | O | SER | 930 | 1.796 | 32.718 | 34.086 | 1.00 | 22.73 |
| ATOM | 838 | N | LYS | 931 | 3.469 | 32.639 | 32.747 | 1.00 | 17.58 |
| ATOM | 839 | CA | LYS | 931 | 3.234 | 33.929 | 32.110 | 1.00 | 16.50 |
| ATOM | 840 | CB | LYS | 931 | 4.568 | 34.472 | 31.621 | 1.00 | 14.67 |
| ATOM | 841 | CG | LYS | 931 | 5.523 | 34.590 | 32.809 | 1.00 | 10.11 |
| ATOM | 842 | CD | LYS | 931 | 4.886 | 35.488 | 33.893 | 1.00 | 17.46 |
| ATOM | 843 | CE | LYS | 931 | 4.747 | 36.972 | 33.491 | 1.00 | 14.85 |
| ATOM | 844 | NZ | LYS | 931 | 6.062 | 37.586 | 33.339 | 1.00 | 22.64 |
| ATOM | 845 | C | LYS | 931 | 2.241 | 33.900 | 30.937 | 1.00 | 20.01 |
| ATOM | 846 | O | LYS | 931 | 2.066 | 34.863 | 30.195 | 1.00 | 19.29 |
| ATOM | 847 | N | ARG | 932 | 1.380 | 32.892 | 30.860 | 1.00 | 22.21 |
| ATOM | 848 | CA | ARG | 932 | 0.395 | 32.774 | 29.749 | 1.00 | 20.74 |
| ATOM | 849 | CB | ARG | 932 | −0.341 | 31.406 | 29.889 | 1.00 | 22.59 |
| ATOM | 850 | CG | ARG | 932 | 0.562 | 30.176 | 29.643 | 1.00 | 19.84 |
| ATOM | 851 | CD | ARG | 932 | 0.975 | 30.212 | 28.173 | 1.00 | 15.47 |
| ATOM | 852 | NE | ARG | 932 | 1.384 | 28.899 | 27.704 | 1.00 | 17.30 |
| ATOM | 853 | CZ | ARG | 932 | 0.453 | 27.957 | 27.479 | 1.00 | 23.01 |
| ATOM | 854 | NH1 | ARG | 932 | 0.858 | 26.754 | 27.021 | 1.00 | 19.75 |
| ATOM | 855 | NH2 | ARG | 932 | −0.889 | 28.205 | 27.685 | 1.00 | 27.63 |
| ATOM | 856 | C | ARG | 932 | −0.600 | 33.896 | 29.709 | 1.00 | 19.71 |
| ATOM | 857 | O | ARG | 932 | −1.052 | 34.387 | 28.674 | 1.00 | 19.83 |
| ATOM | 858 | N | ASN | 933 | −1.057 | 34.154 | 30.922 | 1.00 | 20.75 |
| ATOM | 859 | CA | ASN | 933 | −2.029 | 35.213 | 31.231 | 1.00 | 19.44 |
| ATOM | 860 | CB | ASN | 933 | −2.796 | 34.799 | 32.534 | 1.00 | 19.91 |
| ATOM | 861 | CG | ASN | 933 | −3.968 | 35.740 | 32.734 | 1.00 | 25.34 |
| ATOM | 862 | OD1 | ASN | 933 | −5.007 | 35.778 | 32.096 | 1.00 | 32.98 |
| ATOM | 863 | ND2 | ASN | 933 | −3.836 | 36.746 | 33.517 | 1.00 | 32.96 |
| ATOM | 864 | C | ASN | 933 | −1.236 | 36.508 | 31.427 | 1.00 | 18.77 |
| ATOM | 865 | O | ASN | 933 | −1.778 | 37.501 | 31.895 | 1.00 | 20.59 |
| ATOM | 866 | N | GLU | 934 | 0.080 | 36.551 | 31.151 | 1.00 | 16.50 |
| ATOM | 867 | CA | GLU | 934 | 0.903 | 37.760 | 31.369 | 1.00 | 15.48 |
| ATOM | 868 | CB | GLU | 934 | 1.652 | 37.703 | 32.736 | 1.00 | 17.27 |
| ATOM | 869 | CG | GLU | 934 | 0.814 | 37.866 | 34.045 | 1.00 | 26.32 |
| ATOM | 870 | CD | GLU | 934 | 1.313 | 37.213 | 35.370 | 1.00 | 31.89 |
| ATOM | 871 | OE1 | GLU | 934 | 0.475 | 36.766 | 36.190 | 1.00 | 34.84 |
| ATOM | 872 | OE2 | GLU | 934 | 2.526 | 37.169 | 35.583 | 1.00 | 34.70 |
| ATOM | 873 | C | GLU | 934 | 1.913 | 37.854 | 30.257 | 1.00 | 16.50 |
| ATOM | 874 | O | GLU | 934 | 3.113 | 38.119 | 30.450 | 1.00 | 13.93 |
| ATOM | 875 | N | PHE | 935 | 1.290 | 37.859 | 29.071 | 1.00 | 18.94 |
| ATOM | 876 | CA | PHE | 935 | 1.923 | 37.937 | 27.742 | 1.00 | 22.22 |
| ATOM | 877 | CB | PHE | 935 | 2.139 | 36.463 | 27.153 | 1.00 | 23.60 |
| ATOM | 878 | CG | PHE | 935 | 2.757 | 36.540 | 25.821 | 1.00 | 19.64 |
| ATOM | 879 | CD1 | PHE | 935 | 1.969 | 36.483 | 24.694 | 1.00 | 16.40 |
| ATOM | 880 | CD2 | PHE | 935 | 4.131 | 36.726 | 25.725 | 1.00 | 20.23 |
| ATOM | 881 | CE1 | PHE | 935 | 2.589 | 36.615 | 23.457 | 1.00 | 17.74 |
| ATOM | 882 | CE2 | PHE | 935 | 4.747 | 36.857 | 24.480 | 1.00 | 19.05 |
| ATOM | 883 | CZ | PHE | 935 | 3.964 | 36.798 | 23.345 | 1.00 | 15.52 |
| ATOM | 884 | C | PHE | 935 | 1.141 | 38.794 | 26.703 | 1.00 | 24.21 |
| ATOM | 885 | O | PHE | 935 | −0.082 | 38.809 | 26.600 | 1.00 | 24.17 |
| ATOM | 886 | N | VAL | 936 | 1.919 | 39.496 | 25.879 | 1.00 | 24.70 |
| ATOM | 887 | CA | VAL | 936 | 1.445 | 40.418 | 24.849 | 1.00 | 25.86 |
| ATOM | 888 | CB | VAL | 936 | 1.074 | 41.820 | 25.384 | 1.00 | 27.85 |
| ATOM | 889 | CG1 | VAL | 936 | −0.379 | 42.103 | 25.146 | 1.00 | 28.02 |
| ATOM | 890 | CG2 | VAL | 936 | 1.352 | 41.913 | 26.870 | 1.00 | 31.62 |
| ATOM | 891 | C | VAL | 936 | 2.608 | 40.683 | 23.963 | 1.00 | 27.45 |
| ATOM | 892 | O | VAL | 936 | 3.701 | 40.874 | 24.471 | 1.00 | 29.60 |
| ATOM | 893 | N | PRO | 937 | 2.478 | 40.713 | 22.659 | 1.00 | 29.49 |
| ATOM | 894 | CD | PRO | 937 | 1.437 | 40.018 | 21.919 | 1.00 | 26.87 |
| ATOM | 895 | CA | PRO | 937 | 3.586 | 41.274 | 21.797 | 1.00 | 29.05 |
| ATOM | 896 | CB | PRO | 937 | 3.378 | 40.542 | 20.479 | 1.00 | 28.15 |
| ATOM | 897 | CG | PRO | 937 | 2.389 | 39.437 | 20.896 | 1.00 | 30.32 |
| ATOM | 898 | C | PRO | 937 | 3.618 | 42.811 | 21.660 | 1.00 | 33.07 |
| ATOM | 899 | O | PRO | 937 | 4.240 | 43.355 | 20.748 | 1.00 | 36.47 |
| ATOM | 900 | N | TYR | 938 | 2.803 | 43.461 | 22.445 | 1.00 | 33.52 |
| ATOM | 901 | CA | TYR | 938 | 2.633 | 44.908 | 22.552 | 1.00 | 36.34 |
| ATOM | 902 | CB | TYR | 938 | 1.252 | 45.164 | 23.204 | 1.00 | 33.55 |
| ATOM | 903 | C | TYR | 938 | 3.759 | 45.348 | 23.529 | 1.00 | 43.79 |
| ATOM | 904 | O | TYR | 938 | 3.550 | 46.042 | 24.577 | 1.00 | 44.25 |
| ATOM | 906 | CB | PHE | 999 | 4.464 | 40.578 | 32.665 | 1.00 | 40.01 |
| ATOM | 907 | CG | PHE | 999 | 5.316 | 41.409 | 33.484 | 1.00 | 48.27 |
| ATOM | 908 | CD1 | PHE | 999 | 4.775 | 42.518 | 34.176 | 1.00 | 52.42 |
| ATOM | 909 | CD2 | PHE | 999 | 6.695 | 41.083 | 33.570 | 1.00 | 54.30 |
| ATOM | 910 | CE1 | PHE | 999 | 5.634 | 43.298 | 34.972 | 1.00 | 56.83 |
| ATOM | 911 | CE2 | PHE | 999 | 7.572 | 41.861 | 34.368 | 1.00 | 57.48 |
| ATOM | 912 | CZ | PHE | 999 | 7.023 | 42.970 | 35.063 | 1.00 | 59.76 |
| ATOM | 913 | C | PHE | 999 | 5.452 | 40.809 | 30.465 | 1.00 | 31.92 |
| ATOM | 914 | O | PHE | 999 | 6.477 | 41.497 | 30.324 | 1.00 | 31.70 |
| ATOM | 915 | N | PHE | 999 | 2.882 | 41.274 | 30.947 | 1.00 | 34.46 |
| ATOM | 916 | CA | PHE | 999 | 4.321 | 41.318 | 31.396 | 1.00 | 33.30 |
| ATOM | 917 | N | LEU | 1000 | 5.425 | 39.560 | 29.946 | 1.00 | 29.10 |
| ATOM | 918 | CA | LEU | 1000 | 6.469 | 39.155 | 28.936 | 1.00 | 25.79 |
| ATOM | 919 | CB | LEU | 1000 | 6.589 | 37.628 | 28.870 | 1.00 | 26.71 |
| ATOM | 920 | CG | LEU | 1000 | 7.853 | 36.983 | 29.452 | 1.00 | 26.10 |
| ATOM | 921 | CD1 | LEU | 1000 | 8.401 | 37.688 | 30.741 | 1.00 | 28.56 |
| ATOM | 922 | CD2 | LEU | 1000 | 7.450 | 35.514 | 29.656 | 1.00 | 23.97 |
| ATOM | 923 | C | LEU | 1000 | 6.023 | 39.687 | 27.554 | 1.00 | 22.88 |
| ATOM | 924 | O | LEU | 1000 | 4.829 | 39.908 | 27.340 | 1.00 | 20.05 |
| ATOM | 925 | N | THR | 1001 | 6.913 | 39.828 | 26.571 | 1.00 | 23.18 |
| ATOM | 926 | CA | THR | 1001 | 6.528 | 40.398 | 25.227 | 1.00 | 22.89 |
| ATOM | 927 | CB | THR | 1001 | 6.830 | 41.929 | 25.115 | 1.00 | 21.27 |
| ATOM | 928 | OG1 | THR | 1001 | 8.260 | 42.127 | 25.004 | 1.00 | 25.74 |
| ATOM | 929 | CG2 | THR | 1001 | 6.271 | 42.689 | 26.335 | 1.00 | 13.51 |
| ATOM | 930 | C | THR | 1001 | 7.291 | 39.734 | 24.113 | 1.00 | 22.89 |
| ATOM | 931 | O | THR | 1001 | 8.274 | 39.084 | 24.434 | 1.00 | 25.53 |
| ATOM | 932 | N | LEU | 1002 | 6.977 | 39.840 | 22.831 | 1.00 | 24.27 |
| ATOM | 933 | CA | LEU | 1002 | 7.800 | 39.129 | 21.830 | 1.00 | 25.08 |
| ATOM | 934 | CB | LEU | 1002 | 7.331 | 39.450 | 20.393 | 1.00 | 28.82 |
| ATOM | 935 | CG | LEU | 1002 | 6.636 | 38.225 | 19.672 | 1.00 | 30.66 |
| ATOM | 936 | CD1 | LEU | 1002 | 6.073 | 38.723 | 18.321 | 1.00 | 31.00 |
| ATOM | 937 | CD2 | LEU | 1002 | 7.615 | 37.032 | 19.447 | 1.00 | 28.63 |
| ATOM | 938 | C | LEU | 1002 | 9.272 | 39.448 | 21.938 | 1.00 | 24.86 |
| ATOM | 939 | O | LEU | 1002 | 10.146 | 38.604 | 21.732 | 1.00 | 25.70 |
| ATOM | 940 | N | GLU | 1003 | 9.581 | 40.680 | 22.315 | 1.00 | 25.48 |
| ATOM | 941 | CA | GLU | 1003 | 10.976 | 41.088 | 22.494 | 1.00 | 23.93 |
| ATOM | 942 | CB | GLU | 1003 | 10.990 | 42.542 | 22.956 | 1.00 | 24.85 |
| ATOM | 943 | CG | GLU | 1003 | 12.433 | 43.006 | 22.644 | 1.00 | 30.01 |
| ATOM | 944 | CD | GLU | 1003 | 12.816 | 44.454 | 23.007 | 1.00 | 34.08 |
| ATOM | 945 | OE1 | GLU | 1003 | 12.033 | 45.191 | 23.630 | 1.00 | 34.70 |
| ATOM | 946 | OE2 | GLU | 1003 | 13.949 | 44.815 | 22.651 | 1.00 | 34.12 |
| ATOM | 947 | C | GLU | 1003 | 11.727 | 40.201 | 23.503 | 1.00 | 22.21 |
| ATOM | 948 | O | GLU | 1003 | 12.883 | 39.790 | 23.320 | 1.00 | 20.79 |
| ATOM | 949 | N | HIS | 1004 | 11.068 | 39.932 | 24.640 | 1.00 | 21.60 |
| ATOM | 950 | CA | HIS | 1004 | 11.627 | 39.092 | 25.719 | 1.00 | 20.99 |
| ATOM | 951 | CB | HIS | 1004 | 10.628 | 38.938 | 26.870 | 1.00 | 21.66 |
| ATOM | 952 | CG | HIS | 1004 | 10.442 | 40.112 | 27.728 | 1.00 | 21.59 |
| ATOM | 953 | CD2 | HIS | 1004 | 9.354 | 40.949 | 27.818 | 1.00 | 23.86 |
| ATOM | 954 | ND1 | HIS | 1004 | 11.252 | 40.464 | 28.737 | 1.00 | 25.21 |
| ATOM | 955 | CE1 | HIS | 1004 | 10.704 | 41.483 | 29.456 | 1.00 | 22.61 |
| ATOM | 956 | NE2 | HIS | 1004 | 9.533 | 41.774 | 28.887 | 1.00 | 23.93 |
| ATOM | 957 | C | HIS | 1004 | 11.875 | 37.704 | 25.114 | 1.00 | 24.15 |
| ATOM | 958 | O | HIS | 1004 | 12.967 | 37.141 | 25.217 | 1.00 | 27.39 |
| ATOM | 959 | N | LEU | 1005 | 10.892 | 37.103 | 24.409 | 1.00 | 26.20 |
| ATOM | 960 | CA | LEU | 1005 | 11.031 | 35.757 | 23.764 | 1.00 | 21.29 |
| ATOM | 961 | CB | LEU | 1005 | 9.653 | 35.459 | 23.065 | 1.00 | 17.41 |
| ATOM | 962 | CG | LEU | 1005 | 8.592 | 34.601 | 23.885 | 1.00 | 16.62 |
| ATOM | 963 | CD1 | LEU | 1005 | 8.506 | 34.984 | 25.386 | 1.00 | 10.65 |
| ATOM | 964 | CD2 | LEU | 1005 | 7.264 | 34.707 | 23.120 | 1.00 | 9.60 |
| ATOM | 965 | C | LEU | 1005 | 12.239 | 35.671 | 22.822 | 1.00 | 20.75 |
| ATOM | 966 | O | LEU | 1005 | 12.906 | 34.610 | 22.761 | 1.00 | 23.00 |
| ATOM | 967 | N | ILE | 1006 | 12.546 | 36.743 | 22.036 | 1.00 | 19.62 |
| ATOM | 968 | CA | ILE | 1006 | 13.770 | 36.705 | 21.146 | 1.00 | 19.79 |
| ATOM | 969 | CB | ILE | 1006 | 13.649 | 37.684 | 19.920 | 1.00 | 17.52 |
| ATOM | 970 | CG2 | ILE | 1006 | 14.914 | 37.665 | 19.014 | 1.00 | 13.82 |
| ATOM | 971 | CG1 | ILE | 1006 | 12.431 | 37.211 | 19.103 | 1.00 | 20.39 |
| ATOM | 972 | CD1 | ILE | 1006 | 11.376 | 38.343 | 19.001 | 1.00 | 18.62 |
| ATOM | 973 | C | ILE | 1006 | 15.042 | 37.044 | 21.967 | 1.00 | 15.71 |
| ATOM | 974 | O | ILE | 1006 | 16.143 | 36.559 | 21.723 | 1.00 | 15.24 |
| ATOM | 975 | N | CYS | 1007 | 14.991 | 37.926 | 22.940 | 1.00 | 12.90 |
| ATOM | 976 | CA | CYS | 1007 | 16.195 | 38.161 | 23.809 | 1.00 | 14.43 |
| ATOM | 977 | CB | CYS | 1007 | 15.934 | 39.117 | 25.027 | 1.00 | 14.35 |
| ATOM | 978 | SG | CYS | 1007 | 17.469 | 39.814 | 25.432 | 1.00 | 22.10 |
| ATOM | 979 | C | CYS | 1007 | 16.632 | 36.815 | 24.426 | 1.00 | 11.92 |
| ATOM | 980 | O | CYS | 1007 | 17.780 | 36.396 | 24.297 | 1.00 | 14.55 |
| ATOM | 981 | N | TYR | 1008 | 15.759 | 36.083 | 25.113 | 1.00 | 9.43 |
| ATOM | 982 | CA | TYR | 1008 | 16.121 | 34.770 | 25.694 | 1.00 | 10.07 |
| ATOM | 983 | CB | TYR | 1008 | 14.845 | 34.153 | 26.260 | 1.00 | 8.24 |
| ATOM | 984 | CG | TYR | 1008 | 14.196 | 34.891 | 27.358 | 1.00 | 7.95 |
| ATOM | 985 | CD1 | TYR | 1008 | 12.863 | 34.515 | 27.591 | 1.00 | 6.21 |
| ATOM | 986 | CE1 | TYR | 1008 | 12.139 | 35.125 | 28.655 | 1.00 | 8.81 |
| ATOM | 987 | CD2 | TYR | 1008 | 14.809 | 35.879 | 28.193 | 1.00 | 7.32 |
| ATOM | 988 | CE2 | TYR | 1008 | 14.095 | 36.492 | 29.261 | 1.00 | 4.96 |
| ATOM | 989 | CZ | TYR | 1008 | 12.746 | 36.099 | 29.472 | 1.00 | 7.54 |
| ATOM | 990 | OH | TYR | 1008 | 11.929 | 36.709 | 30.410 | 1.00 | 6.11 |
| ATOM | 991 | C | TYR | 1008 | 16.740 | 33.833 | 24.617 | 1.00 | 10.83 |
| ATOM | 992 | O | TYR | 1008 | 17.811 | 33.239 | 24.722 | 1.00 | 11.71 |
| ATOM | 993 | N | SER | 1009 | 16.067 | 33.630 | 23.510 | 1.00 | 9.19 |
| ATOM | 994 | CA | SER | 1009 | 16.620 | 32.791 | 22.422 | 1.00 | 8.22 |
| ATOM | 995 | CB | SER | 1009 | 15.637 | 32.909 | 21.204 | 1.00 | 10.29 |
| ATOM | 996 | OG | SER | 1009 | 14.238 | 32.648 | 21.502 | 1.00 | 9.38 |
| ATOM | 997 | C | SER | 1009 | 18.011 | 33.254 | 22.000 | 1.00 | 7.39 |
| ATOM | 998 | O | SER | 1009 | 18.917 | 32.533 | 21.600 | 1.00 | 7.81 |
| ATOM | 999 | N | PHE | 1010 | 18.200 | 34.555 | 21.896 | 1.00 | 10.34 |
| ATOM | 1000 | CA | PHE | 1010 | 19.516 | 35.149 | 21.446 | 1.00 | 12.36 |
| ATOM | 1001 | CB | PHE | 1010 | 19.281 | 36.714 | 21.330 | 1.00 | 9.03 |
| ATOM | 1002 | CG | PHE | 1010 | 20.549 | 37.438 | 21.150 | 1.00 | 7.58 |
| ATOM | 1003 | CD1 | PHE | 1010 | 20.907 | 38.439 | 22.074 | 1.00 | 9.07 |
| ATOM | 1004 | CD2 | PHE | 1010 | 21.395 | 37.145 | 20.058 | 1.00 | 6.81 |
| ATOM | 1005 | CE1 | PHE | 1010 | 22.125 | 39.135 | 21.869 | 1.00 | 7.66 |
| ATOM | 1006 | CE2 | PHE | 1010 | 22.622 | 37.841 | 19.838 | 1.00 | 6.56 |
| ATOM | 1007 | CZ | PHE | 1010 | 22.969 | 38.840 | 20.758 | 1.00 | 7.83 |
| ATOM | 1008 | C | PHE | 1010 | 20.635 | 34.752 | 22.447 | 1.00 | 13.46 |
| ATOM | 1009 | O | PHE | 1010 | 21.729 | 34.218 | 22.133 | 1.00 | 13.49 |
| ATOM | 1010 | N | GLN | 1011 | 20.316 | 35.045 | 23.712 | 1.00 | 11.51 |
| ATOM | 1011 | CA | GLN | 1011 | 21.184 | 34.700 | 24.795 | 1.00 | 12.47 |
| ATOM | 1012 | CB | GLN | 1011 | 20.514 | 35.076 | 26.070 | 1.00 | 15.52 |
| ATOM | 1013 | CG | GLN | 1011 | 20.419 | 36.611 | 26.342 | 1.00 | 15.85 |
| ATOM | 1014 | CD | GLN | 1011 | 20.072 | 36.846 | 27.827 | 1.00 | 16.04 |
| ATOM | 1015 | OE1 | GLN | 1011 | 20.793 | 36.553 | 28.768 | 1.00 | 12.73 |
| ATOM | 1016 | NE2 | GLN | 1011 | 18.941 | 37.380 | 28.163 | 1.00 | 14.27 |
| ATOM | 1017 | C | GLN | 1011 | 21.452 | 33.230 | 24.773 | 1.00 | 15.13 |
| ATOM | 1018 | O | GLN | 1011 | 22.616 | 32.802 | 24.694 | 1.00 | 19.14 |
| ATOM | 1019 | N | VAL | 1012 | 20.454 | 32.341 | 24.735 | 1.00 | 13.27 |
| ATOM | 1020 | CA | VAL | 1012 | 20.834 | 30.887 | 24.747 | 1.00 | 9.31 |
| ATOM | 1021 | CB | VAL | 1012 | 19.624 | 29.925 | 24.612 | 1.00 | 6.49 |
| ATOM | 1022 | CG1 | VAL | 1012 | 20.154 | 28.502 | 24.585 | 1.00 | 2.73 |
| ATOM | 1023 | CG2 | VAL | 1012 | 18.669 | 30.016 | 25.753 | 1.00 | 2.00 |
| ATOM | 1024 | C | VAL | 1012 | 21.755 | 30.594 | 23.586 | 1.00 | 12.25 |
| ATOM | 1025 | O | VAL | 1012 | 22.578 | 29.683 | 23.659 | 1.00 | 19.11 |
| ATOM | 1026 | N | ALA | 1013 | 21.696 | 31.326 | 22.456 | 1.00 | 13.53 |
| ATOM | 1027 | CA | ALA | 1013 | 22.592 | 31.044 | 21.317 | 1.00 | 6.32 |
| ATOM | 1028 | CB | ALA | 1013 | 21.964 | 31.650 | 20.149 | 1.00 | 8.83 |
| ATOM | 1029 | C | ALA | 1013 | 23.950 | 31.614 | 21.573 | 1.00 | 8.78 |
| ATOM | 1030 | O | ALA | 1013 | 25.010 | 31.077 | 21.229 | 1.00 | 12.21 |
| ATOM | 1031 | N | LYS | 1014 | 24.025 | 32.751 | 22.211 | 1.00 | 12.34 |
| ATOM | 1032 | CA | LYS | 1014 | 25.339 | 33.323 | 22.558 | 1.00 | 8.79 |
| ATOM | 1033 | CB | LYS | 1014 | 25.147 | 34.700 | 23.247 | 1.00 | 11.20 |
| ATOM | 1034 | CG | LYS | 1014 | 25.368 | 36.069 | 22.506 | 1.00 | 10.64 |
| ATOM | 1035 | CD | LYS | 1014 | 26.804 | 36.593 | 22.371 | 1.00 | 13.71 |
| ATOM | 1036 | CE | LYS | 1014 | 27.942 | 35.672 | 21.717 | 1.00 | 26.46 |
| ATOM | 1037 | NZ | LYS | 1014 | 28.610 | 34.454 | 22.391 | 1.00 | 21.27 |
| ATOM | 1038 | C | LYS | 1014 | 25.965 | 32.319 | 23.478 | 1.00 | 8.87 |
| ATOM | 1039 | O | LYS | 1014 | 27.056 | 31.811 | 23.231 | 1.00 | 12.10 |
| ATOM | 1040 | N | GLY | 1015 | 25.277 | 31.920 | 24.546 | 1.00 | 8.80 |
| ATOM | 1041 | CA | GLY | 1015 | 25.804 | 30.912 | 25.519 | 1.00 | 10.47 |
| ATOM | 1042 | C | GLY | 1015 | 26.298 | 29.605 | 24.856 | 1.00 | 12.36 |
| ATOM | 1043 | O | GLY | 1015 | 27.364 | 29.065 | 25.211 | 1.00 | 14.38 |
| ATOM | 1044 | N | MET | 1016 | 25.533 | 29.054 | 23.883 | 1.00 | 12.50 |
| ATOM | 1045 | CA | MET | 1016 | 25.922 | 27.846 | 23.160 | 1.00 | 7.67 |
| ATOM | 1046 | CB | MET | 1016 | 24.721 | 27.421 | 22.479 | 1.00 | 8.23 |
| ATOM | 1047 | CG | MET | 1016 | 23.792 | 26.768 | 23.495 | 1.00 | 6.76 |
| ATOM | 1048 | SD | MET | 1016 | 24.330 | 25.226 | 24.289 | 1.00 | 11.91 |
| ATOM | 1049 | CE | MET | 1016 | 25.166 | 24.010 | 23.256 | 1.00 | 10.16 |
| ATOM | 1050 | C | MET | 1016 | 27.064 | 28.100 | 22.230 | 1.00 | 10.60 |
| ATOM | 1051 | O | MET | 1016 | 27.912 | 27.220 | 22.062 | 1.00 | 10.59 |
| ATOM | 1052 | N | GLU | 1017 | 27.118 | 29.269 | 21.545 | 1.00 | 13.76 |
| ATOM | 1053 | CA | GLU | 1017 | 28.302 | 29.612 | 20.664 | 1.00 | 12.29 |
| ATOM | 1054 | CB | GLU | 1017 | 28.213 | 31.002 | 20.082 | 1.00 | 12.78 |
| ATOM | 1055 | CG | GLU | 1017 | 29.421 | 31.459 | 19.235 | 1.00 | 13.10 |
| ATOM | 1056 | CD | GLU | 1017 | 29.215 | 32.906 | 18.740 | 1.00 | 17.83 |
| ATOM | 1057 | OE1 | GLU | 1017 | 28.630 | 33.731 | 19.444 | 1.00 | 16.75 |
| ATOM | 1058 | OE2 | GLU | 1017 | 29.684 | 33.242 | 17.640 | 1.00 | 18.08 |
| ATOM | 1059 | C | GLU | 1017 | 29.555 | 29.613 | 21.518 | 1.00 | 13.94 |
| ATOM | 1060 | O | GLU | 1017 | 30.652 | 29.214 | 21.167 | 1.00 | 16.64 |
| ATOM | 1061 | N | PHE | 1018 | 29.454 | 30.144 | 22.704 | 1.00 | 15.68 |
| ATOM | 1062 | CA | PHE | 1018 | 30.568 | 30.121 | 23.655 | 1.00 | 14.51 |
| ATOM | 1063 | CB | PHE | 1018 | 30.105 | 30.943 | 24.805 | 1.00 | 13.53 |
| ATOM | 1064 | CG | PHE | 1018 | 31.129 | 30.839 | 25.825 | 1.00 | 18.42 |
| ATOM | 1065 | CD1 | PHE | 1018 | 30.919 | 29.998 | 26.944 | 1.00 | 18.19 |
| ATOM | 1066 | CD2 | PHE | 1018 | 32.332 | 31.550 | 25.660 | 1.00 | 19.24 |
| ATOM | 1067 | CE1 | PHE | 1018 | 31.920 | 29.857 | 27.896 | 1.00 | 15.99 |
| ATOM | 1068 | CE2 | PHE | 1018 | 33.338 | 31.410 | 26.625 | 1.00 | 18.92 |
| ATOM | 1069 | CZ | PHE | 1018 | 33.116 | 30.566 | 27.717 | 1.00 | 16.36 |
| ATOM | 1070 | C | PHE | 1018 | 30.883 | 28.651 | 24.048 | 1.00 | 16.71 |
| ATOM | 1071 | O | PHE | 1018 | 31.974 | 28.125 | 23.800 | 1.00 | 18.86 |
| ATOM | 1072 | N | LEU | 1019 | 29.934 | 27.876 | 24.607 | 1.00 | 17.49 |
| ATOM | 1073 | CA | LEU | 1019 | 30.242 | 26.475 | 24.942 | 1.00 | 15.03 |
| ATOM | 1074 | CB | LEU | 1019 | 29.008 | 25.565 | 25.256 | 1.00 | 17.22 |
| ATOM | 1075 | CG | LEU | 1019 | 28.375 | 25.421 | 26.663 | 1.00 | 20.16 |
| ATOM | 1076 | CD1 | LEU | 1019 | 27.357 | 26.528 | 26.884 | 1.00 | 24.87 |
| ATOM | 1077 | CD2 | LEU | 1019 | 27.542 | 24.122 | 26.767 | 1.00 | 19.99 |
| ATOM | 1078 | C | LEU | 1019 | 30.916 | 25.814 | 23.774 | 1.00 | 15.18 |
| ATOM | 1079 | O | LEU | 1019 | 31.941 | 25.197 | 24.027 | 1.00 | 13.03 |
| ATOM | 1080 | N | ALA | 1020 | 30.457 | 26.017 | 22.498 | 1.00 | 17.41 |
| ATOM | 1081 | CA | ALA | 1020 | 31.064 | 25.345 | 21.301 | 1.00 | 17.29 |
| ATOM | 1082 | CB | ALA | 1020 | 30.333 | 25.705 | 20.020 | 1.00 | 16.66 |
| ATOM | 1083 | C | ALA | 1020 | 32.507 | 25.713 | 21.116 | 1.00 | 17.88 |
| ATOM | 1084 | O | ALA | 1020 | 33.343 | 24.940 | 20.621 | 1.00 | 18.07 |
| ATOM | 1085 | N | SER | 1021 | 32.819 | 26.944 | 21.513 | 1.00 | 19.46 |
| ATOM | 1086 | CA | SER | 1021 | 34.225 | 27.457 | 21.446 | 1.00 | 19.60 |
| ATOM | 1087 | CB | SER | 1021 | 34.357 | 28.964 | 21.651 | 1.00 | 17.68 |
| ATOM | 1088 | OG | SER | 1021 | 34.318 | 29.319 | 23.002 | 1.00 | 13.49 |
| ATOM | 1089 | C | SER | 1021 | 35.152 | 26.867 | 22.485 | 1.00 | 20.90 |
| ATOM | 1090 | O | SER | 1021 | 36.392 | 26.898 | 22.372 | 1.00 | 20.30 |
| ATOM | 1091 | N | ARG | 1022 | 34.577 | 26.399 | 23.592 | 1.00 | 21.73 |
| ATOM | 1092 | CA | ARG | 1022 | 35.462 | 25.737 | 24.626 | 1.00 | 23.29 |
| ATOM | 1093 | CB | ARG | 1022 | 34.811 | 25.955 | 26.002 | 1.00 | 19.06 |
| ATOM | 1094 | CG | ARG | 1022 | 34.436 | 27.421 | 26.140 | 1.00 | 17.26 |
| ATOM | 1095 | CD | ARG | 1022 | 35.450 | 28.194 | 26.855 | 1.00 | 19.37 |
| ATOM | 1096 | NE | ARG | 1022 | 36.787 | 27.968 | 26.300 | 1.00 | 26.51 |
| ATOM | 1097 | CZ | ARG | 1022 | 37.830 | 27.635 | 27.105 | 1.00 | 30.51 |
| ATOM | 1098 | NH1 | ARG | 1022 | 39.058 | 27.449 | 26.648 | 1.00 | 30.76 |
| ATOM | 1099 | NH2 | ARG | 1022 | 37.691 | 27.423 | 28.403 | 1.00 | 34.40 |
| ATOM | 1100 | C | ARG | 1022 | 35.666 | 24.233 | 24.338 | 1.00 | 24.70 |
| ATOM | 1101 | O | ARG | 1022 | 36.190 | 23.419 | 25.129 | 1.00 | 28.75 |
| ATOM | 1102 | N | LYS | 1023 | 35.209 | 23.898 | 23.135 | 1.00 | 24.60 |
| ATOM | 1103 | CA | LYS | 1023 | 35.168 | 22.586 | 22.507 | 1.00 | 25.75 |
| ATOM | 1104 | CB | LYS | 1023 | 36.572 | 21.973 | 22.331 | 1.00 | 28.02 |
| ATOM | 1105 | CG | LYS | 1023 | 37.416 | 22.396 | 21.088 | 1.00 | 32.08 |
| ATOM | 1106 | CD | LYS | 1023 | 38.674 | 23.188 | 21.514 | 1.00 | 39.21 |
| ATOM | 1107 | CE | LYS | 1023 | 39.415 | 23.966 | 20.377 | 1.00 | 43.64 |
| ATOM | 1108 | NZ | LYS | 1023 | 38.492 | 24.745 | 19.504 | 1.00 | 46.60 |
| ATOM | 1109 | C | LYS | 1023 | 34.309 | 21.647 | 23.330 | 1.00 | 27.20 |
| ATOM | 1110 | O | LYS | 1023 | 34.536 | 20.430 | 23.341 | 1.00 | 29.16 |
| ATOM | 1111 | N | CYS | 1024 | 33.216 | 22.170 | 23.943 | 1.00 | 25.20 |
| ATOM | 1112 | CA | CYS | 1024 | 32.290 | 21.350 | 24.752 | 1.00 | 22.97 |
| ATOM | 1113 | CB | CYS | 1024 | 32.024 | 21.919 | 26.204 | 1.00 | 24.60 |
| ATOM | 1114 | SG | CYS | 1024 | 33.496 | 21.834 | 27.256 | 1.00 | 27.46 |
| ATOM | 1115 | C | CYS | 1024 | 30.949 | 21.214 | 24.145 | 1.00 | 22.69 |
| ATOM | 1116 | O | CYS | 1024 | 30.597 | 22.004 | 23.266 | 1.00 | 23.17 |
| ATOM | 1117 | N | ILE | 1025 | 30.172 | 20.167 | 24.530 | 1.00 | 23.95 |
| ATOM | 1118 | CA | ILE | 1025 | 28.772 | 20.045 | 24.021 | 1.00 | 24.01 |
| ATOM | 1119 | CB | ILE | 1025 | 28.483 | 18.938 | 22.926 | 1.00 | 24.45 |
| ATOM | 1120 | CG2 | ILE | 1025 | 29.331 | 19.235 | 21.694 | 1.00 | 22.47 |
| ATOM | 1121 | CG1 | ILE | 1025 | 28.693 | 17.541 | 23.452 | 1.00 | 24.05 |
| ATOM | 1122 | CD1 | ILE | 1025 | 27.946 | 16.741 | 22.399 | 1.00 | 23.93 |
| ATOM | 1123 | C | ILE | 1025 | 27.811 | 19.724 | 25.168 | 1.00 | 24.34 |
| ATOM | 1124 | O | ILE | 1025 | 28.232 | 19.170 | 26.175 | 1.00 | 24.92 |
| ATOM | 1125 | N | HIS | 1026 | 26.528 | 20.119 | 25.082 | 1.00 | 21.91 |
| ATOM | 1126 | CA | HIS | 1026 | 25.531 | 19.880 | 26.130 | 1.00 | 21.71 |
| ATOM | 1127 | CB | HIS | 1026 | 24.482 | 21.060 | 26.080 | 1.00 | 15.99 |
| ATOM | 1128 | CG | HIS | 1026 | 23.711 | 21.212 | 27.344 | 1.00 | 12.17 |
| ATOM | 1129 | CD2 | HIS | 1026 | 23.826 | 22.288 | 28.229 | 1.00 | 11.55 |
| ATOM | 1130 | ND1 | HIS | 1026 | 22.791 | 20.350 | 27.874 | 1.00 | 9.32 |
| ATOM | 1131 | CE1 | HIS | 1026 | 22.344 | 20.900 | 29.055 | 1.00 | 9.74 |
| ATOM | 1132 | NE2 | HIS | 1026 | 22.982 | 22.082 | 29.273 | 1.00 | 9.81 |
| ATOM | 1133 | C | HIS | 1026 | 24.778 | 18.500 | 26.173 | 1.00 | 24.15 |
| ATOM | 1134 | O | HIS | 1026 | 24.281 | 18.101 | 27.235 | 1.00 | 28.76 |
| ATOM | 1135 | N | ARG | 1027 | 24.492 | 17.789 | 25.073 | 1.00 | 23.53 |
| ATOM | 1136 | CA | ARG | 1027 | 23.777 | 16.463 | 25.126 | 1.00 | 21.22 |
| ATOM | 1137 | CB | ARG | 1027 | 24.661 | 15.419 | 25.825 | 1.00 | 25.96 |
| ATOM | 1138 | CG | ARG | 1027 | 26.030 | 15.349 | 25.153 | 1.00 | 32.50 |
| ATOM | 1139 | CD | ARG | 1027 | 27.073 | 14.472 | 25.899 | 1.00 | 36.29 |
| ATOM | 1140 | NE | ARG | 1027 | 28.316 | 14.615 | 25.117 | 1.00 | 39.52 |
| ATOM | 1141 | CZ | ARG | 1027 | 29.229 | 13.651 | 24.926 | 1.00 | 41.51 |
| ATOM | 1142 | NH1 | ARG | 1027 | 30.277 | 13.953 | 24.188 | 1.00 | 44.25 |
| ATOM | 1143 | NH2 | ARG | 1027 | 29.163 | 12.414 | 25.418 | 1.00 | 43.71 |
| ATOM | 1144 | C | ARG | 1027 | 22.407 | 16.453 | 25.806 | 1.00 | 17.92 |
| ATOM | 1145 | O | ARG | 1027 | 21.758 | 15.413 | 25.910 | 1.00 | 19.10 |
| ATOM | 1146 | N | ASP | 1028 | 21.900 | 17.621 | 26.186 | 1.00 | 15.64 |
| ATOM | 1147 | CA | ASP | 1028 | 20.553 | 17.737 | 26.830 | 1.00 | 15.23 |
| ATOM | 1148 | CB | ASP | 1028 | 20.546 | 17.245 | 28.325 | 1.00 | 14.18 |
| ATOM | 1149 | CG | ASP | 1028 | 19.131 | 17.007 | 28.843 | 1.00 | 14.38 |
| ATOM | 1150 | OD1 | ASP | 1028 | 18.931 | 16.472 | 29.925 | 1.00 | 16.10 |
| ATOM | 1151 | OD2 | ASP | 1028 | 18.190 | 17.325 | 28.152 | 1.00 | 16.65 |
| ATOM | 1152 | C | ASP | 1028 | 20.005 | 19.145 | 26.865 | 1.00 | 14.95 |
| ATOM | 1153 | O | ASP | 1028 | 19.554 | 19.655 | 27.896 | 1.00 | 18.74 |
| ATOM | 1154 | N | LEU | 1029 | 20.189 | 19.886 | 25.800 | 1.00 | 13.58 |
| ATOM | 1155 | CA | LEU | 1029 | 19.651 | 21.246 | 25.798 | 1.00 | 10.75 |
| ATOM | 1156 | CB | LEU | 1029 | 20.367 | 22.024 | 24.602 | 1.00 | 9.09 |
| ATOM | 1157 | CG | LEU | 1029 | 20.078 | 23.537 | 24.517 | 1.00 | 8.22 |
| ATOM | 1158 | CD1 | LEU | 1029 | 20.337 | 24.394 | 25.802 | 1.00 | 3.38 |
| ATOM | 1159 | CD2 | LEU | 1029 | 21.001 | 23.939 | 23.403 | 1.00 | 7.48 |
| ATOM | 1160 | C | LEU | 1029 | 18.103 | 21.273 | 25.737 | 1.00 | 8.77 |
| ATOM | 1161 | O | LEU | 1029 | 17.402 | 20.773 | 24.871 | 1.00 | 9.76 |
| ATOM | 1162 | N | ALA | 1030 | 17.523 | 21.958 | 26.654 | 1.00 | 9.29 |
| ATOM | 1163 | CA | ALA | 1030 | 16.107 | 22.109 | 26.746 | 1.00 | 7.15 |
| ATOM | 1164 | CB | ALA | 1030 | 15.526 | 20.914 | 27.417 | 1.00 | 8.65 |
| ATOM | 1165 | C | ALA | 1030 | 15.908 | 23.296 | 27.649 | 1.00 | 8.44 |
| ATOM | 1166 | O | ALA | 1030 | 16.748 | 23.592 | 28.475 | 1.00 | 10.85 |
| ATOM | 1167 | N | ALA | 1031 | 14.753 | 23.923 | 27.625 | 1.00 | 12.49 |
| ATOM | 1168 | CA | ALA | 1031 | 14.365 | 25.097 | 28.445 | 1.00 | 12.73 |
| ATOM | 1169 | CB | ALA | 1031 | 12.948 | 25.663 | 27.982 | 1.00 | 10.17 |
| ATOM | 1170 | C | ALA | 1031 | 14.286 | 24.769 | 29.931 | 1.00 | 12.95 |
| ATOM | 1171 | O | ALA | 1031 | 13.990 | 25.611 | 30.770 | 1.00 | 14.59 |
| ATOM | 1172 | N | ARG | 1032 | 14.366 | 23.466 | 30.208 | 1.00 | 13.85 |
| ATOM | 1173 | CA | ARG | 1032 | 14.326 | 22.995 | 31.575 | 1.00 | 15.78 |
| ATOM | 1174 | CB | ARG | 1032 | 14.183 | 21.467 | 31.850 | 1.00 | 13.57 |
| ATOM | 1175 | CG | ARG | 1032 | 13.618 | 20.390 | 31.039 | 1.00 | 10.77 |
| ATOM | 1176 | CD | ARG | 1032 | 14.523 | 19.160 | 31.032 | 1.00 | 14.09 |
| ATOM | 1177 | NE | ARG | 1032 | 14.204 | 18.738 | 29.673 | 1.00 | 27.96 |
| ATOM | 1178 | CZ | ARG | 1032 | 14.826 | 17.874 | 28.890 | 1.00 | 30.22 |
| ATOM | 1179 | NH1 | ARG | 1032 | 14.324 | 17.709 | 27.704 | 1.00 | 34.01 |
| ATOM | 1180 | NH2 | ARG | 1032 | 15.881 | 17.177 | 29.190 | 1.00 | 31.48 |
| ATOM | 1181 | C | ARG | 1032 | 15.720 | 23.263 | 32.060 | 1.00 | 15.85 |
| ATOM | 1182 | O | ARG | 1032 | 15.945 | 23.521 | 33.230 | 1.00 | 22.30 |
| ATOM | 1183 | N | ASN | 1033 | 16.710 | 23.067 | 31.217 | 1.00 | 13.17 |
| ATOM | 1184 | CA | ASN | 1033 | 18.098 | 23.359 | 31.626 | 1.00 | 12.39 |
| ATOM | 1185 | CB | ASN | 1033 | 18.990 | 22.368 | 30.928 | 1.00 | 12.29 |
| ATOM | 1186 | CG | ASN | 1033 | 18.859 | 21.067 | 31.706 | 1.00 | 10.75 |
| ATOM | 1187 | OD1 | ASN | 1033 | 18.642 | 20.957 | 32.904 | 1.00 | 13.32 |
| ATOM | 1188 | ND2 | ASN | 1033 | 19.015 | 19.989 | 31.019 | 1.00 | 8.36 |
| ATOM | 1189 | C | ASN | 1033 | 18.555 | 24.813 | 31.335 | 1.00 | 10.12 |
| ATOM | 1190 | O | ASN | 1033 | 19.707 | 25.173 | 31.091 | 1.00 | 8.07 |
| ATOM | 1191 | N | ILE | 1034 | 17.631 | 25.716 | 31.549 | 1.00 | 9.65 |
| ATOM | 1192 | CA | ILE | 1034 | 17.878 | 27.152 | 31.280 | 1.00 | 9.59 |
| ATOM | 1193 | CB | ILE | 1034 | 17.166 | 27.705 | 29.881 | 1.00 | 8.72 |
| ATOM | 1194 | CG2 | ILE | 1034 | 17.421 | 29.223 | 29.747 | 1.00 | 4.16 |
| ATOM | 1195 | CG1 | ILE | 1034 | 17.727 | 27.030 | 28.599 | 1.00 | 2.00 |
| ATOM | 1196 | CD1 | ILE | 1034 | 19.183 | 27.284 | 28.284 | 1.00 | 2.00 |
| ATOM | 1197 | C | ILE | 1034 | 17.293 | 27.890 | 32.469 | 1.00 | 7.90 |
| ATOM | 1198 | O | ILE | 1034 | 16.117 | 27.767 | 32.801 | 1.00 | 10.47 |
| ATOM | 1199 | N | LEU | 1035 | 18.179 | 28.459 | 33.221 | 1.00 | 6.72 |
| ATOM | 1200 | CA | LEU | 1035 | 17.726 | 29.211 | 34.376 | 1.00 | 11.07 |
| ATOM | 1201 | CB | LEU | 1035 | 18.862 | 29.030 | 35.408 | 1.00 | 9.95 |
| ATOM | 1202 | CG | LEU | 1035 | 18.992 | 27.664 | 35.962 | 1.00 | 7.14 |
| ATOM | 1203 | CD1 | LEU | 1035 | 20.175 | 27.736 | 36.878 | 1.00 | 10.75 |
| ATOM | 1204 | CD2 | LEU | 1035 | 17.905 | 27.268 | 36.899 | 1.00 | 8.13 |
| ATOM | 1205 | C | LEU | 1035 | 17.407 | 30.706 | 34.031 | 1.00 | 8.32 |
| ATOM | 1206 | O | LEU | 1035 | 18.108 | 31.416 | 33.276 | 1.00 | 7.45 |
| ATOM | 1207 | N | LEU | 1036 | 16.380 | 31.264 | 34.580 | 1.00 | 7.32 |
| ATOM | 1208 | CA | LEU | 1036 | 16.133 | 32.699 | 34.227 | 1.00 | 14.45 |
| ATOM | 1209 | CB | LEU | 1036 | 14.569 | 32.917 | 33.996 | 1.00 | 11.59 |
| ATOM | 1210 | CG | LEU | 1036 | 14.322 | 34.394 | 33.755 | 1.00 | 9.60 |
| ATOM | 1211 | CD1 | LEU | 1036 | 14.950 | 34.839 | 32.424 | 1.00 | 14.16 |
| ATOM | 1212 | CD2 | LEU | 1036 | 12.854 | 34.605 | 33.921 | 1.00 | 12.93 |
| ATOM | 1213 | C | LEU | 1036 | 16.722 | 33.588 | 35.367 | 1.00 | 17.60 |
| ATOM | 1214 | O | LEU | 1036 | 16.429 | 33.362 | 36.563 | 1.00 | 17.87 |
| ATOM | 1215 | N | SER | 1037 | 17.352 | 34.701 | 35.052 | 1.00 | 16.95 |
| ATOM | 1216 | CA | SER | 1037 | 17.976 | 35.482 | 36.108 | 1.00 | 21.60 |
| ATOM | 1217 | CB | SER | 1037 | 19.392 | 35.519 | 35.795 | 1.00 | 21.24 |
| ATOM | 1218 | OG | SER | 1037 | 19.979 | 35.171 | 37.036 | 1.00 | 29.94 |
| ATOM | 1219 | C | SER | 1037 | 17.402 | 36.872 | 36.303 | 1.00 | 25.47 |
| ATOM | 1220 | O | SER | 1037 | 16.330 | 37.149 | 35.796 | 1.00 | 29.11 |
| ATOM | 1221 | N | GLU | 1038 | 17.924 | 37.780 | 37.135 | 1.00 | 27.41 |
| ATOM | 1222 | CA | GLU | 1038 | 17.296 | 39.158 | 37.241 | 1.00 | 26.12 |
| ATOM | 1223 | CB | GLU | 1038 | 17.870 | 39.911 | 38.452 | 1.00 | 27.66 |
| ATOM | 1224 | CG | GLU | 1038 | 18.094 | 39.050 | 39.741 | 1.00 | 33.68 |
| ATOM | 1225 | CD | GLU | 1038 | 19.366 | 38.145 | 39.594 | 1.00 | 37.77 |
| ATOM | 1226 | OE1 | GLU | 1038 | 19.297 | 36.889 | 39.804 | 1.00 | 34.62 |
| ATOM | 1227 | OE2 | GLU | 1038 | 20.442 | 38.730 | 39.295 | 1.00 | 42.16 |
| ATOM | 1228 | C | GLU | 1038 | 17.616 | 39.896 | 35.906 | 1.00 | 27.11 |
| ATOM | 1229 | O | GLU | 1038 | 18.441 | 39.382 | 35.177 | 1.00 | 23.68 |
| ATOM | 1230 | N | LYS | 1039 | 16.991 | 41.065 | 35.525 | 1.00 | 27.53 |
| ATOM | 1231 | CA | LYS | 1039 | 17.366 | 41.710 | 34.209 | 1.00 | 27.41 |
| ATOM | 1232 | CB | LYS | 1039 | 18.853 | 42.086 | 33.906 | 1.00 | 30.99 |
| ATOM | 1233 | CG | LYS | 1039 | 19.751 | 42.337 | 32.569 | 1.00 | 30.73 |
| ATOM | 1234 | CD | LYS | 1039 | 19.416 | 43.069 | 31.176 | 1.00 | 29.53 |
| ATOM | 1235 | CE | LYS | 1039 | 20.291 | 42.721 | 29.849 | 1.00 | 34.26 |
| ATOM | 1236 | NZ | LYS | 1039 | 20.200 | 41.371 | 29.155 | 1.00 | 19.84 |
| ATOM | 1237 | C | LYS | 1039 | 17.000 | 40.852 | 33.054 | 1.00 | 28.40 |
| ATOM | 1238 | O | LYS | 1039 | 17.224 | 41.276 | 31.966 | 1.00 | 31.49 |
| ATOM | 1239 | N | ASN | 1040 | 16.430 | 39.646 | 33.093 | 1.00 | 30.89 |
| ATOM | 1240 | CA | ASN | 1040 | 16.154 | 38.808 | 31.840 | 1.00 | 27.95 |
| ATOM | 1241 | CB | ASN | 1040 | 15.517 | 39.598 | 30.638 | 1.00 | 26.17 |
| ATOM | 1242 | CG | ASN | 1040 | 14.183 | 40.052 | 31.152 | 1.00 | 29.72 |
| ATOM | 1243 | OD1 | ASN | 1040 | 13.195 | 39.336 | 31.369 | 1.00 | 29.29 |
| ATOM | 1244 | ND2 | ASN | 1040 | 14.113 | 41.328 | 31.349 | 1.00 | 30.67 |
| ATOM | 1245 | C | ASN | 1040 | 17.345 | 38.104 | 31.257 | 1.00 | 25.37 |
| ATOM | 1246 | O | ASN | 1040 | 17.330 | 37.744 | 30.080 | 1.00 | 26.26 |
| ATOM | 1247 | N | VAL | 1041 | 18.400 | 38.029 | 32.111 | 1.00 | 20.70 |
| ATOM | 1248 | CA | VAL | 1041 | 19.606 | 37.287 | 31.734 | 1.00 | 17.90 |
| ATOM | 1249 | CB | VAL | 1041 | 20.733 | 37.692 | 32.649 | 1.00 | 17.23 |
| ATOM | 1250 | CG1 | VAL | 1041 | 21.956 | 36.941 | 32.252 | 1.00 | 14.94 |
| ATOM | 1251 | CG2 | VAL | 1041 | 21.042 | 39.147 | 32.506 | 1.00 | 15.55 |
| ATOM | 1252 | C | VAL | 1041 | 19.290 | 35.778 | 31.930 | 1.00 | 20.35 |
| ATOM | 1253 | O | VAL | 1041 | 18.772 | 35.319 | 32.960 | 1.00 | 20.35 |
| ATOM | 1254 | N | VAL | 1042 | 19.425 | 35.010 | 30.862 | 1.00 | 20.41 |
| ATOM | 1255 | CA | VAL | 1042 | 19.149 | 33.578 | 30.889 | 1.00 | 17.47 |
| ATOM | 1256 | CB | VAL | 1042 | 18.381 | 33.387 | 29.566 | 1.00 | 17.98 |
| ATOM | 1257 | CG1 | VAL | 1042 | 18.805 | 32.180 | 28.828 | 1.00 | 20.72 |
| ATOM | 1258 | CG2 | VAL | 1042 | 16.895 | 33.186 | 29.873 | 1.00 | 16.82 |
| ATOM | 1259 | C | VAL | 1042 | 20.461 | 32.849 | 31.065 | 1.00 | 16.59 |
| ATOM | 1260 | O | VAL | 1042 | 21.514 | 33.233 | 30.532 | 1.00 | 21.58 |
| ATOM | 1261 | N | LYS | 1043 | 20.423 | 31.735 | 31.722 | 1.00 | 14.85 |
| ATOM | 1262 | CA | LYS | 1043 | 21.647 | 30.982 | 31.944 | 1.00 | 12.92 |
| ATOM | 1263 | CB | LYS | 1043 | 22.001 | 31.039 | 33.452 | 1.00 | 13.81 |
| ATOM | 1264 | CG | LYS | 1043 | 21.986 | 32.402 | 34.096 | 1.00 | 8.01 |
| ATOM | 1265 | CD | LYS | 1043 | 23.236 | 33.167 | 33.866 | 1.00 | 7.29 |
| ATOM | 1266 | CE | LYS | 1043 | 23.051 | 34.494 | 34.602 | 1.00 | 7.58 |
| ATOM | 1267 | NZ | LYS | 1043 | 24.297 | 35.261 | 34.387 | 1.00 | 9.84 |
| ATOM | 1268 | C | LYS | 1043 | 21.607 | 29.499 | 31.526 | 1.00 | 12.86 |
| ATOM | 1269 | O | LYS | 1043 | 20.828 | 28.742 | 32.069 | 1.00 | 14.73 |
| ATOM | 1270 | N | ILE | 1044 | 22.429 | 28.985 | 30.629 | 1.00 | 14.98 |
| ATOM | 1271 | CA | ILE | 1044 | 22.457 | 27.539 | 30.281 | 1.00 | 17.13 |
| ATOM | 1272 | CB | ILE | 1044 | 23.360 | 27.319 | 29.037 | 1.00 | 16.46 |
| ATOM | 1273 | CG2 | ILE | 1044 | 23.267 | 25.834 | 28.613 | 1.00 | 12.44 |
| ATOM | 1274 | CG1 | ILE | 1044 | 22.890 | 28.203 | 27.877 | 1.00 | 12.12 |
| ATOM | 1275 | CD1 | ILE | 1044 | 23.995 | 28.271 | 26.836 | 1.00 | 13.17 |
| ATOM | 1276 | C | ILE | 1044 | 22.993 | 26.645 | 31.449 | 1.00 | 20.60 |
| ATOM | 1277 | O | ILE | 1044 | 24.139 | 26.785 | 31.926 | 1.00 | 19.81 |
| ATOM | 1278 | N | CYS | 1045 | 22.164 | 25.756 | 32.030 | 1.00 | 19.57 |
| ATOM | 1279 | CA | CYS | 1045 | 22.667 | 24.866 | 33.113 | 1.00 | 14.26 |
| ATOM | 1280 | CB | CYS | 1045 | 21.934 | 25.290 | 34.342 | 1.00 | 8.88 |
| ATOM | 1281 | SG | CYS | 1045 | 20.439 | 24.488 | 34.525 | 1.00 | 11.76 |
| ATOM | 1282 | C | CYS | 1045 | 22.495 | 23.389 | 32.807 | 1.00 | 14.86 |
| ATOM | 1283 | O | CYS | 1045 | 22.250 | 22.960 | 31.659 | 1.00 | 16.38 |
| ATOM | 1284 | N | ASP | 1046 | 22.658 | 22.527 | 33.769 | 1.00 | 14.35 |
| ATOM | 1285 | CA | ASP | 1046 | 22.464 | 21.070 | 33.592 | 1.00 | 11.64 |
| ATOM | 1286 | CB | ASP | 1046 | 23.774 | 20.484 | 33.021 | 1.00 | 11.65 |
| ATOM | 1287 | CG | ASP | 1046 | 23.687 | 18.985 | 32.623 | 1.00 | 14.87 |
| ATOM | 1288 | OD1 | ASP | 1046 | 24.717 | 18.329 | 32.606 | 1.00 | 19.21 |
| ATOM | 1289 | OD2 | ASP | 1046 | 22.633 | 18.421 | 32.331 | 1.00 | 15.98 |
| ATOM | 1290 | C | ASP | 1046 | 22.073 | 20.459 | 34.928 | 1.00 | 11.06 |
| ATOM | 1291 | O | ASP | 1046 | 22.933 | 20.242 | 35.767 | 1.00 | 11.13 |
| ATOM | 1292 | N | PHE | 1047 | 20.803 | 20.297 | 35.246 | 1.00 | 12.22 |
| ATOM | 1293 | CA | PHE | 1047 | 20.420 | 19.705 | 36.538 | 1.00 | 17.30 |
| ATOM | 1294 | CB | PHE | 1047 | 18.861 | 19.694 | 36.534 | 1.00 | 16.14 |
| ATOM | 1295 | CG | PHE | 1047 | 18.244 | 21.031 | 36.763 | 1.00 | 19.11 |
| ATOM | 1296 | CD1 | PHE | 1047 | 18.631 | 22.185 | 36.025 | 1.00 | 24.17 |
| ATOM | 1297 | CD2 | PHE | 1047 | 17.229 | 21.153 | 37.712 | 1.00 | 18.47 |
| ATOM | 1298 | CE1 | PHE | 1047 | 18.032 | 23.445 | 36.206 | 1.00 | 22.86 |
| ATOM | 1299 | CE2 | PHE | 1047 | 16.609 | 22.405 | 37.918 | 1.00 | 21.80 |
| ATOM | 1300 | CZ | PHE | 1047 | 17.009 | 23.535 | 37.170 | 1.00 | 20.84 |
| ATOM | 1301 | C | PHE | 1047 | 21.068 | 18.274 | 36.807 | 1.00 | 21.49 |
| ATOM | 1302 | O | PHE | 1047 | 21.422 | 17.898 | 37.930 | 1.00 | 21.38 |
| ATOM | 1303 | N | GLY | 1048 | 21.370 | 17.486 | 35.744 | 1.00 | 25.00 |
| ATOM | 1304 | CA | GLY | 1048 | 21.911 | 16.111 | 35.811 | 1.00 | 26.41 |
| ATOM | 1305 | C | GLY | 1048 | 21.194 | 15.280 | 36.897 | 1.00 | 31.10 |
| ATOM | 1306 | O | GLY | 1048 | 19.967 | 15.064 | 37.005 | 1.00 | 29.45 |
| ATOM | 1307 | N | LEU | 1049 | 22.069 | 14.848 | 37.795 | 1.00 | 33.84 |
| ATOM | 1308 | CA | LEU | 1049 | 21.749 | 14.045 | 38.992 | 1.00 | 33.89 |
| ATOM | 1309 | CB | LEU | 1049 | 23.117 | 13.856 | 39.729 | 1.00 | 36.60 |
| ATOM | 1310 | CG | LEU | 1049 | 23.908 | 12.508 | 39.502 | 1.00 | 36.06 |
| ATOM | 1311 | CD1 | LEU | 1049 | 23.631 | 11.936 | 38.110 | 1.00 | 31.81 |
| ATOM | 1312 | CD2 | LEU | 1049 | 25.420 | 12.757 | 39.781 | 1.00 | 35.06 |
| ATOM | 1313 | C | LEU | 1049 | 20.651 | 14.707 | 39.841 | 1.00 | 33.57 |
| ATOM | 1314 | O | LEU | 1049 | 19.881 | 14.008 | 40.509 | 1.00 | 32.74 |
| ATOM | 1315 | N | ALA | 1050 | 20.590 | 16.032 | 39.904 | 1.00 | 32.32 |
| ATOM | 1316 | CA | ALA | 1050 | 19.527 | 16.678 | 40.660 | 1.00 | 32.18 |
| ATOM | 1317 | CB | ALA | 1050 | 20.012 | 18.062 | 40.964 | 1.00 | 32.22 |
| ATOM | 1318 | C | ALA | 1050 | 18.154 | 16.693 | 39.950 | 1.00 | 31.65 |
| ATOM | 1319 | O | ALA | 1050 | 17.227 | 17.395 | 40.335 | 1.00 | 30.44 |
| ATOM | 1320 | N | ARG | 1051 | 18.015 | 16.060 | 38.783 | 1.00 | 32.10 |
| ATOM | 1321 | CA | ARG | 1051 | 16.677 | 15.960 | 38.138 | 1.00 | 34.15 |
| ATOM | 1322 | CB | ARG | 1051 | 16.813 | 16.240 | 36.606 | 1.00 | 34.48 |
| ATOM | 1323 | CG | ARG | 1051 | 15.613 | 16.324 | 35.651 | 1.00 | 34.50 |
| ATOM | 1324 | CD | ARG | 1051 | 15.809 | 17.615 | 34.774 | 1.00 | 40.37 |
| ATOM | 1325 | NE | ARG | 1051 | 14.981 | 18.698 | 35.347 | 1.00 | 41.89 |
| ATOM | 1326 | CZ | ARG | 1051 | 15.021 | 19.978 | 34.943 | 1.00 | 46.60 |
| ATOM | 1327 | NH1 | ARG | 1051 | 14.165 | 20.879 | 35.521 | 1.00 | 49.08 |
| ATOM | 1328 | NH2 | ARG | 1051 | 15.963 | 20.380 | 34.066 | 1.00 | 43.64 |
| ATOM | 1329 | C | ARG | 1051 | 16.129 | 14.526 | 38.374 | 1.00 | 34.43 |
| ATOM | 1330 | O | ARG | 1051 | 16.843 | 13.534 | 38.432 | 1.00 | 33.40 |
| ATOM | 1331 | N | ASP | 1052 | 14.821 | 14.392 | 38.579 | 1.00 | 36.26 |
| ATOM | 1332 | CA | ASP | 1052 | 14.119 | 13.093 | 38.789 | 1.00 | 35.81 |
| ATOM | 1333 | CB | ASP | 1052 | 12.668 | 13.377 | 39.117 | 1.00 | 39.58 |
| ATOM | 1334 | C | ASP | 1052 | 14.141 | 12.355 | 37.468 | 1.00 | 36.55 |
| ATOM | 1335 | O | ASP | 1052 | 13.085 | 11.980 | 36.986 | 1.00 | 37.40 |
| ATOM | 1336 | N | ILE | 1053 | 15.277 | 11.914 | 36.966 | 1.00 | 38.54 |
| ATOM | 1337 | CA | ILE | 1053 | 15.345 | 11.307 | 35.616 | 1.00 | 39.37 |
| ATOM | 1338 | CB | ILE | 1053 | 16.729 | 10.609 | 35.616 | 1.00 | 41.22 |
| ATOM | 1339 | CG2 | ILE | 1053 | 16.668 | 9.249 | 36.408 | 1.00 | 45.25 |
| ATOM | 1340 | CG1 | ILE | 1053 | 17.198 | 10.427 | 34.178 | 1.00 | 39.81 |
| ATOM | 1341 | CD1 | ILE | 1053 | 18.206 | 9.263 | 34.004 | 1.00 | 36.48 |
| ATOM | 1342 | C | ILE | 1053 | 14.137 | 10.393 | 35.235 | 1.00 | 40.72 |
| ATOM | 1343 | O | ILE | 1053 | 13.649 | 10.500 | 34.109 | 1.00 | 43.18 |
| ATOM | 1344 | N | TYR | 1054 | 13.625 | 9.508 | 36.159 | 1.00 | 42.36 |
| ATOM | 1345 | CA | TYR | 1054 | 12.462 | 8.582 | 35.923 | 1.00 | 41.24 |
| ATOM | 1346 | CB | TYR | 1054 | 12.535 | 7.341 | 36.862 | 1.00 | 39.36 |
| ATOM | 1347 | C | TYR | 1054 | 11.126 | 9.308 | 36.148 | 1.00 | 40.53 |
| ATOM | 1348 | O | TYR | 1054 | 10.265 | 9.303 | 35.281 | 1.00 | 44.69 |
| ATOM | 1349 | N | LYS | 1055 | 10.875 | 9.969 | 37.275 | 1.00 | 37.36 |
| ATOM | 1350 | CA | LYS | 1055 | 9.591 | 10.663 | 37.361 | 1.00 | 34.68 |
| ATOM | 1351 | CB | LYS | 1055 | 9.426 | 11.280 | 38.718 | 1.00 | 37.20 |
| ATOM | 1352 | C | LYS | 1055 | 9.454 | 11.789 | 36.318 | 1.00 | 33.87 |
| ATOM | 1353 | O | LYS | 1055 | 8.377 | 12.340 | 36.134 | 1.00 | 34.44 |
| ATOM | 1354 | N | ASP | 1056 | 10.499 | 12.216 | 35.633 | 1.00 | 33.29 |
| ATOM | 1355 | CA | ASP | 1056 | 10.371 | 13.315 | 34.667 | 1.00 | 33.11 |
| ATOM | 1356 | CB | ASP | 1056 | 11.728 | 14.080 | 34.567 | 1.00 | 33.89 |
| ATOM | 1357 | CG | ASP | 1056 | 11.704 | 15.503 | 34.021 | 1.00 | 34.80 |
| ATOM | 1358 | OD1 | ASP | 1056 | 12.684 | 16.211 | 34.195 | 1.00 | 35.70 |
| ATOM | 1359 | OD2 | ASP | 1056 | 10.738 | 15.908 | 33.409 | 1.00 | 37.37 |
| ATOM | 1360 | C | ASP | 1056 | 9.930 | 12.851 | 33.298 | 1.00 | 34.02 |
| ATOM | 1361 | O | ASP | 1056 | 10.700 | 12.301 | 32.511 | 1.00 | 33.10 |
| ATOM | 1362 | N | PRO | 1057 | 8.692 | 13.166 | 32.914 | 1.00 | 33.69 |
| ATOM | 1363 | CD | PRO | 1057 | 7.750 | 14.112 | 33.554 | 1.00 | 32.54 |
| ATOM | 1364 | CA | PRO | 1057 | 8.175 | 12.699 | 31.621 | 1.00 | 33.71 |
| ATOM | 1365 | CB | PRO | 1057 | 6.750 | 13.198 | 31.639 | 1.00 | 33.12 |
| ATOM | 1366 | CG | PRO | 1057 | 6.422 | 13.581 | 33.066 | 1.00 | 32.13 |
| ATOM | 1367 | C | PRO | 1057 | 8.989 | 13.161 | 30.384 | 1.00 | 35.16 |
| ATOM | 1368 | O | PRO | 1057 | 8.691 | 12.821 | 29.219 | 1.00 | 35.30 |
| ATOM | 1369 | N | ASP | 1058 | 9.956 | 14.073 | 30.585 | 1.00 | 37.33 |
| ATOM | 1370 | CA | ASP | 1058 | 10.788 | 14.510 | 29.437 | 1.00 | 38.28 |
| ATOM | 1371 | CB | ASP | 1058 | 11.566 | 15.760 | 29.803 | 1.00 | 41.23 |
| ATOM | 1372 | CG | ASP | 1058 | 10.576 | 16.867 | 30.028 | 1.00 | 42.94 |
| ATOM | 1373 | OD1 | ASP | 1058 | 10.320 | 17.229 | 31.146 | 1.00 | 48.53 |
| ATOM | 1374 | OD2 | ASP | 1058 | 10.034 | 17.379 | 29.088 | 1.00 | 43.27 |
| ATOM | 1375 | C | ASP | 1058 | 11.763 | 13.438 | 28.985 | 1.00 | 39.06 |
| ATOM | 1376 | O | ASP | 1058 | 12.518 | 13.572 | 28.018 | 1.00 | 38.16 |
| ATOM | 1377 | N | TYR | 1059 | 11.750 | 12.372 | 29.763 | 1.00 | 38.97 |
| ATOM | 1378 | CA | TYR | 1059 | 12.599 | 11.237 | 29.496 | 1.00 | 40.37 |
| ATOM | 1379 | CB | TYR | 1059 | 13.664 | 11.137 | 30.620 | 1.00 | 39.09 |
| ATOM | 1380 | CG | TYR | 1059 | 14.416 | 12.377 | 30.788 | 1.00 | 34.71 |
| ATOM | 1381 | CD1 | TYR | 1059 | 13.822 | 13.393 | 31.508 | 1.00 | 35.47 |
| ATOM | 1382 | CE1 | TYR | 1059 | 14.467 | 14.609 | 31.716 | 1.00 | 33.07 |
| ATOM | 1383 | CD2 | TYR | 1059 | 15.695 | 12.566 | 30.252 | 1.00 | 33.13 |
| ATOM | 1384 | CE2 | TYR | 1059 | 16.351 | 13.788 | 30.457 | 1.00 | 30.95 |
| ATOM | 1385 | CZ | TYR | 1059 | 15.723 | 14.807 | 31.194 | 1.00 | 32.59 |
| ATOM | 1386 | OH | TYR | 1059 | 16.309 | 16.035 | 31.427 | 1.00 | 30.28 |
| ATOM | 1387 | C | TYR | 1059 | 11.817 | 9.930 | 29.385 | 1.00 | 42.81 |
| ATOM | 1388 | O | TYR | 1059 | 11.001 | 9.538 | 30.232 | 1.00 | 44.61 |
| ATOM | 1389 | N | VAL | 1060 | 12.043 | 9.264 | 28.276 | 1.00 | 44.99 |
| ATOM | 1390 | CA | VAL | 1060 | 11.391 | 7.956 | 28.031 | 1.00 | 46.04 |
| ATOM | 1391 | CB | VAL | 1060 | 10.388 | 8.160 | 26.751 | 1.00 | 46.37 |
| ATOM | 1392 | CG1 | VAL | 1060 | 10.389 | 9.610 | 26.263 | 1.00 | 45.91 |
| ATOM | 1393 | CG2 | VAL | 1060 | 10.788 | 7.335 | 25.536 | 1.00 | 47.67 |
| ATOM | 1394 | C | VAL | 1060 | 12.630 | 6.996 | 27.923 | 1.00 | 45.16 |
| ATOM | 1395 | O | VAL | 1060 | 13.781 | 7.432 | 27.651 | 1.00 | 42.16 |
| ATOM | 1397 | CB | LEU | 1067 | 16.619 | 9.107 | 28.602 | 1.00 | 16.21 |
| ATOM | 1398 | C | LEU | 1067 | 15.638 | 10.753 | 26.994 | 1.00 | 18.14 |
| ATOM | 1399 | O | LEU | 1067 | 14.446 | 10.779 | 27.239 | 1.00 | 21.96 |
| ATOM | 1400 | N | LEU | 1067 | 17.334 | 8.922 | 26.070 | 1.00 | 15.93 |
| ATOM | 1401 | CA | LEU | 1067 | 16.247 | 9.336 | 27.052 | 1.00 | 17.25 |
| ATOM | 1402 | N | PRO | 1068 | 16.173 | 11.962 | 26.708 | 1.00 | 18.19 |
| ATOM | 1403 | CD | PRO | 1068 | 17.492 | 12.493 | 26.998 | 1.00 | 18.82 |
| ATOM | 1404 | CA | PRO | 1068 | 15.342 | 13.168 | 26.405 | 1.00 | 19.76 |
| ATOM | 1405 | CB | PRO | 1068 | 16.205 | 14.379 | 26.607 | 1.00 | 18.69 |
| ATOM | 1406 | CG | PRO | 1068 | 17.527 | 13.804 | 26.181 | 1.00 | 15.97 |
| ATOM | 1407 | C | PRO | 1068 | 14.844 | 13.164 | 24.978 | 1.00 | 22.47 |
| ATOM | 1408 | O | PRO | 1068 | 14.841 | 14.160 | 24.262 | 1.00 | 24.05 |
| ATOM | 1409 | N | LEU | 1069 | 14.308 | 12.051 | 24.559 | 1.00 | 25.80 |
| ATOM | 1410 | CA | LEU | 1069 | 13.836 | 11.872 | 23.152 | 1.00 | 27.31 |
| ATOM | 1411 | CB | LEU | 1069 | 12.868 | 10.655 | 23.128 | 1.00 | 29.14 |
| ATOM | 1412 | CG | LEU | 1069 | 13.527 | 9.272 | 23.165 | 1.00 | 26.96 |
| ATOM | 1413 | CD1 | LEU | 1069 | 14.817 | 9.214 | 22.363 | 1.00 | 28.41 |
| ATOM | 1414 | CD2 | LEU | 1069 | 13.853 | 8.974 | 24.582 | 1.00 | 30.06 |
| ATOM | 1415 | C | LEU | 1069 | 13.187 | 13.028 | 22.346 | 1.00 | 26.28 |
| ATOM | 1416 | O | LEU | 1069 | 13.545 | 13.243 | 21.184 | 1.00 | 25.91 |
| ATOM | 1417 | N | LYS | 1070 | 12.192 | 13.748 | 22.863 | 1.00 | 23.83 |
| ATOM | 1418 | CA | LYS | 1070 | 11.597 | 14.877 | 22.084 | 1.00 | 24.33 |
| ATOM | 1419 | CB | LYS | 1070 | 10.502 | 15.512 | 22.884 | 1.00 | 19.23 |
| ATOM | 1420 | CG | LYS | 1070 | 9.224 | 14.727 | 22.930 | 1.00 | 17.54 |
| ATOM | 1421 | CD | LYS | 1070 | 8.661 | 15.393 | 24.157 | 1.00 | 15.01 |
| ATOM | 1422 | CE | LYS | 1070 | 7.154 | 15.423 | 24.149 | 1.00 | 19.99 |
| ATOM | 1423 | NZ | LYS | 1070 | 6.640 | 16.008 | 25.397 | 1.00 | 22.56 |
| ATOM | 1424 | C | LYS | 1070 | 12.567 | 16.028 | 21.650 | 1.00 | 24.16 |
| ATOM | 1425 | O | LYS | 1070 | 12.229 | 16.901 | 20.847 | 1.00 | 22.41 |
| ATOM | 1426 | N | TRP | 1071 | 13.749 | 16.022 | 22.259 | 1.00 | 22.16 |
| ATOM | 1427 | CA | TRP | 1071 | 14.852 | 16.945 | 22.059 | 1.00 | 19.76 |
| ATOM | 1428 | CB | TRP | 1071 | 15.377 | 17.316 | 23.394 | 1.00 | 19.72 |
| ATOM | 1429 | CG | TRP | 1071 | 14.491 | 18.253 | 24.036 | 1.00 | 20.08 |
| ATOM | 1430 | CD2 | TRP | 1071 | 13.375 | 18.035 | 24.901 | 1.00 | 19.07 |
| ATOM | 1431 | CE2 | TRP | 1071 | 12.853 | 19.314 | 25.203 | 1.00 | 17.22 |
| ATOM | 1432 | CE3 | TRP | 1071 | 12.728 | 16.895 | 25.429 | 1.00 | 18.68 |
| ATOM | 1433 | CD1 | TRP | 1071 | 14.614 | 19.637 | 23.877 | 1.00 | 20.86 |
| ATOM | 1434 | NE1 | TRP | 1071 | 13.619 | 20.248 | 24.592 | 1.00 | 21.10 |
| ATOM | 1435 | CZ2 | TRP | 1071 | 11.737 | 19.449 | 26.032 | 1.00 | 20.35 |
| ATOM | 1436 | CZ3 | TRP | 1071 | 11.602 | 17.041 | 26.251 | 1.00 | 14.98 |
| ATOM | 1437 | CH2 | TRP | 1071 | 11.132 | 18.308 | 26.565 | 1.00 | 17.89 |
| ATOM | 1438 | C | TRP | 1071 | 15.985 | 16.331 | 21.246 | 1.00 | 24.07 |
| ATOM | 1439 | O | TRP | 1071 | 16.851 | 17.041 | 20.706 | 1.00 | 24.77 |
| ATOM | 1440 | N | MET | 1072 | 16.153 | 15.004 | 21.216 | 1.00 | 24.19 |
| ATOM | 1441 | CA | MET | 1072 | 17.263 | 14.515 | 20.361 | 1.00 | 24.85 |
| ATOM | 1442 | CB | MET | 1072 | 17.755 | 13.146 | 20.848 | 1.00 | 26.93 |
| ATOM | 1443 | CG | MET | 1072 | 18.209 | 13.146 | 22.290 | 1.00 | 29.18 |
| ATOM | 1444 | SD | MET | 1072 | 17.226 | 11.925 | 23.121 | 1.00 | 29.81 |
| ATOM | 1445 | CE | MET | 1072 | 18.537 | 10.717 | 22.702 | 1.00 | 29.51 |
| ATOM | 1446 | C | MET | 1072 | 16.998 | 14.397 | 18.847 | 1.00 | 25.38 |
| ATOM | 1447 | O | MET | 1072 | 16.013 | 13.955 | 18.260 | 1.00 | 26.31 |
| ATOM | 1448 | N | ALA | 1073 | 17.995 | 14.769 | 18.145 | 1.00 | 26.67 |
| ATOM | 1449 | CA | ALA | 1073 | 17.990 | 14.724 | 16.695 | 1.00 | 30.94 |
| ATOM | 1450 | CB | ALA | 1073 | 19.269 | 15.412 | 16.288 | 1.00 | 27.93 |
| ATOM | 1451 | C | ALA | 1073 | 17.918 | 13.299 | 16.153 | 1.00 | 33.20 |
| ATOM | 1452 | O | ALA | 1073 | 18.507 | 12.387 | 16.757 | 1.00 | 34.47 |
| ATOM | 1453 | N | PRO | 1074 | 17.393 | 13.054 | 14.938 | 1.00 | 35.07 |
| ATOM | 1454 | CD | PRO | 1074 | 17.203 | 13.984 | 13.798 | 1.00 | 34.57 |
| ATOM | 1455 | CA | PRO | 1074 | 17.243 | 11.642 | 14.524 | 1.00 | 33.65 |
| ATOM | 1456 | CB | PRO | 1074 | 16.528 | 11.782 | 13.174 | 1.00 | 35.65 |
| ATOM | 1457 | CG | PRO | 1074 | 17.127 | 13.060 | 12.562 | 1.00 | 35.25 |
| ATOM | 1458 | C | PRO | 1074 | 18.641 | 11.019 | 14.507 | 1.00 | 31.99 |
| ATOM | 1459 | O | PRO | 1074 | 18.947 | 9.949 | 15.009 | 1.00 | 32.29 |
| ATOM | 1460 | N | GLU | 1075 | 19.598 | 11.785 | 14.082 | 1.00 | 32.33 |
| ATOM | 1461 | CA | GLU | 1075 | 20.971 | 11.280 | 14.014 | 1.00 | 32.52 |
| ATOM | 1462 | CB | GLU | 1075 | 21.871 | 12.408 | 13.501 | 1.00 | 30.10 |
| ATOM | 1463 | CG | GLU | 1075 | 21.332 | 13.820 | 13.617 | 1.00 | 29.26 |
| ATOM | 1464 | CD | GLU | 1075 | 22.350 | 14.669 | 14.297 | 1.00 | 33.78 |
| ATOM | 1465 | OE1 | GLU | 1075 | 21.935 | 15.533 | 15.048 | 1.00 | 34.15 |
| ATOM | 1466 | OE2 | GLU | 1075 | 23.555 | 14.506 | 14.092 | 1.00 | 40.63 |
| ATOM | 1467 | C | GLU | 1075 | 21.507 | 10.711 | 15.315 | 1.00 | 33.53 |
| ATOM | 1468 | O | GLU | 1075 | 22.383 | 9.852 | 15.298 | 1.00 | 32.69 |
| ATOM | 1469 | N | THR | 1076 | 20.987 | 11.158 | 16.456 | 1.00 | 33.04 |
| ATOM | 1470 | CA | THR | 1076 | 21.447 | 10.672 | 17.793 | 1.00 | 35.38 |
| ATOM | 1471 | CB | THR | 1076 | 21.341 | 11.752 | 18.873 | 1.00 | 33.16 |
| ATOM | 1472 | OG1 | THR | 1076 | 22.330 | 12.722 | 18.572 | 1.00 | 34.57 |
| ATOM | 1473 | OG2 | THR | 1076 | 21.512 | 11.227 | 20.287 | 1.00 | 32.98 |
| ATOM | 1474 | C | THR | 1076 | 20.631 | 9.517 | 18.298 | 1.00 | 39.51 |
| ATOM | 1475 | O | THR | 1076 | 21.084 | 8.630 | 19.022 | 1.00 | 42.55 |
| ATOM | 1476 | N | ILE | 1077 | 19.328 | 9.585 | 18.029 | 1.00 | 40.32 |
| ATOM | 1477 | CA | ILE | 1077 | 18.403 | 8.510 | 18.462 | 1.00 | 37.59 |
| ATOM | 1478 | CB | ILE | 1077 | 16.937 | 8.920 | 18.085 | 1.00 | 34.04 |
| ATOM | 1479 | CG2 | ILE | 1077 | 15.962 | 7.939 | 18.726 | 1.00 | 31.86 |
| ATOM | 1480 | CG1 | ILE | 1077 | 16.614 | 10.347 | 18.547 | 1.00 | 29.32 |
| ATOM | 1481 | CD1 | ILE | 1077 | 15.305 | 10.845 | 17.916 | 1.00 | 31.98 |
| ATOM | 1482 | C | ILE | 1077 | 18.874 | 7.279 | 17.679 | 1.00 | 39.48 |
| ATOM | 1483 | O | ILE | 1077 | 19.414 | 6.313 | 18.198 | 1.00 | 40.38 |
| ATOM | 1484 | N | PHE | 1078 | 18.816 | 7.411 | 16.364 | 1.00 | 41.61 |
| ATOM | 1485 | CA | PHE | 1078 | 19.216 | 6.340 | 15.457 | 1.00 | 42.85 |
| ATOM | 1486 | CB | PHE | 1078 | 18.747 | 6.792 | 14.052 | 1.00 | 45.20 |
| ATOM | 1487 | CG | PHE | 1078 | 17.249 | 6.950 | 14.040 | 1.00 | 47.36 |
| ATOM | 1488 | CD1 | PHE | 1078 | 16.648 | 8.220 | 14.165 | 1.00 | 48.67 |
| ATOM | 1489 | CD2 | PHE | 1078 | 16.403 | 5.824 | 13.888 | 1.00 | 48.07 |
| ATOM | 1490 | CE1 | PHE | 1078 | 15.248 | 8.367 | 14.136 | 1.00 | 48.08 |
| ATOM | 1491 | CE2 | PHE | 1078 | 15.003 | 5.962 | 13.856 | 1.00 | 45.18 |
| ATOM | 1492 | CZ | PHE | 1078 | 14.444 | 7.231 | 13.979 | 1.00 | 44.85 |
| ATOM | 1493 | C | PHE | 1078 | 20.715 | 6.021 | 15.554 | 1.00 | 42.63 |
| ATOM | 1494 | O | PHE | 1078 | 21.099 | 4.882 | 15.334 | 1.00 | 46.08 |
| ATOM | 1495 | N | ASP | 1079 | 21.621 | 6.945 | 15.881 | 1.00 | 41.10 |
| ATOM | 1496 | CA | ASP | 1079 | 23.068 | 6.579 | 16.001 | 1.00 | 39.23 |
| ATOM | 1497 | CB | ASP | 1079 | 23.749 | 7.208 | 14.809 | 1.00 | 40.84 |
| ATOM | 1498 | CG | ASP | 1079 | 23.424 | 6.305 | 13.679 | 1.00 | 43.23 |
| ATOM | 1499 | OD1 | ASP | 1079 | 22.291 | 6.317 | 13.202 | 1.00 | 46.96 |
| ATOM | 1500 | OD2 | ASP | 1079 | 24.336 | 5.579 | 13.307 | 1.00 | 48.67 |
| ATOM | 1501 | C | ASP | 1079 | 23.911 | 6.861 | 17.292 | 1.00 | 37.59 |
| ATOM | 1502 | O | ASP | 1079 | 25.147 | 6.821 | 17.305 | 1.00 | 31.47 |
| ATOM | 1503 | N | ARG | 1080 | 23.262 | 7.204 | 18.395 | 1.00 | 35.50 |
| ATOM | 1504 | CA | ARG | 1080 | 23.920 | 7.504 | 19.675 | 1.00 | 37.84 |
| ATOM | 1505 | CB | ARG | 1080 | 24.260 | 6.138 | 20.311 | 1.00 | 40.66 |
| ATOM | 1506 | C | ARG | 1080 | 25.148 | 8.431 | 19.485 | 1.00 | 36.95 |
| ATOM | 1507 | O | ARG | 1080 | 26.071 | 8.530 | 20.299 | 1.00 | 38.21 |
| ATOM | 1508 | N | VAL | 1081 | 25.155 | 9.224 | 18.414 | 1.00 | 38.12 |
| ATOM | 1509 | CA | VAL | 1081 | 26.274 | 10.147 | 18.165 | 1.00 | 37.82 |
| ATOM | 1510 | CB | VAL | 1081 | 26.663 | 10.275 | 16.586 | 1.00 | 39.93 |
| ATOM | 1511 | CG1 | VAL | 1081 | 25.994 | 11.438 | 15.822 | 1.00 | 41.26 |
| ATOM | 1512 | CG2 | VAL | 1081 | 28.226 | 10.511 | 16.504 | 1.00 | 45.80 |
| ATOM | 1513 | C | VAL | 1081 | 25.874 | 11.464 | 18.723 | 1.00 | 34.37 |
| ATOM | 1514 | O | VAL | 1081 | 24.731 | 11.846 | 18.533 | 1.00 | 32.84 |
| ATOM | 1515 | N | TYR | 1082 | 26.734 | 12.077 | 19.537 | 1.00 | 32.85 |
| ATOM | 1516 | CA | TYR | 1082 | 26.411 | 13.395 | 20.096 | 1.00 | 28.88 |
| ATOM | 1517 | CB | TYR | 1082 | 26.408 | 13.263 | 21.618 | 1.00 | 29.78 |
| ATOM | 1518 | CG | TYR | 1082 | 25.267 | 12.501 | 22.151 | 1.00 | 29.56 |
| ATOM | 1519 | CD1 | TYR | 1082 | 25.303 | 11.134 | 22.490 | 1.00 | 31.41 |
| ATOM | 1520 | CE1 | TYR | 1082 | 24.132 | 10.538 | 23.056 | 1.00 | 31.06 |
| ATOM | 1521 | CD2 | TYR | 1082 | 24.109 | 13.217 | 22.379 | 1.00 | 29.37 |
| ATOM | 1522 | CE2 | TYR | 1082 | 22.971 | 12.645 | 22.940 | 1.00 | 29.60 |
| ATOM | 1523 | CZ | TYR | 1082 | 22.958 | 11.305 | 23.285 | 1.00 | 29.64 |
| ATOM | 1524 | OH | TYR | 1082 | 21.787 | 10.865 | 23.894 | 1.00 | 28.12 |
| ATOM | 1525 | C | TYR | 1082 | 27.413 | 14.461 | 19.622 | 1.00 | 29.31 |
| ATOM | 1526 | O | TYR | 1082 | 28.620 | 14.239 | 19.624 | 1.00 | 29.70 |
| ATOM | 1527 | N | THR | 1083 | 26.922 | 15.625 | 19.172 | 1.00 | 28.05 |
| ATOM | 1528 | CA | THR | 1083 | 27.694 | 16.787 | 18.652 | 1.00 | 24.04 |
| ATOM | 1529 | CB | THR | 1083 | 27.890 | 16.817 | 17.113 | 1.00 | 27.53 |
| ATOM | 1530 | OG1 | THR | 1083 | 26.715 | 17.453 | 16.466 | 1.00 | 30.63 |
| ATOM | 1531 | CG2 | THR | 1083 | 28.037 | 15.398 | 16.548 | 1.00 | 27.92 |
| ATOM | 1532 | C | THR | 1083 | 26.948 | 18.065 | 18.915 | 1.00 | 22.06 |
| ATOM | 1533 | O | THR | 1083 | 25.829 | 18.104 | 19.413 | 1.00 | 22.72 |
| ATOM | 1534 | N | ILE | 1084 | 27.522 | 19.159 | 18.483 | 1.00 | 22.51 |
| ATOM | 1535 | CA | ILE | 1084 | 26.960 | 20.516 | 18.620 | 1.00 | 24.42 |
| ATOM | 1536 | CB | ILE | 1084 | 27.987 | 21.629 | 18.336 | 1.00 | 25.77 |
| ATOM | 1537 | CG2 | ILE | 1084 | 27.563 | 22.787 | 19.295 | 1.00 | 21.98 |
| ATOM | 1538 | CG1 | ILE | 1084 | 29.448 | 21.308 | 18.685 | 1.00 | 29.43 |
| ATOM | 1539 | CD1 | ILE | 1084 | 30.288 | 20.059 | 18.210 | 1.00 | 35.16 |
| ATOM | 1540 | C | ILE | 1084 | 25.840 | 20.678 | 17.586 | 1.00 | 26.52 |
| ATOM | 1541 | O | ILE | 1084 | 24.811 | 21.327 | 17.779 | 1.00 | 26.59 |
| ATOM | 1542 | N | GLN | 1085 | 26.026 | 20.064 | 16.407 | 1.00 | 28.92 |
| ATOM | 1543 | CA | GLN | 1085 | 24.978 | 20.170 | 15.374 | 1.00 | 27.89 |
| ATOM | 1544 | CB | GLN | 1085 | 25.444 | 19.597 | 13.980 | 1.00 | 27.47 |
| ATOM | 1545 | CG | GLN | 1085 | 26.253 | 20.699 | 13.198 | 1.00 | 26.63 |
| ATOM | 1546 | CD | GLN | 1085 | 25.750 | 22.170 | 13.425 | 1.00 | 28.58 |
| ATOM | 1547 | OE1 | GLN | 1085 | 26.361 | 23.008 | 14.077 | 1.00 | 24.93 |
| ATOM | 1548 | NE2 | GLN | 1085 | 24.585 | 22.568 | 12.982 | 1.00 | 31.05 |
| ATOM | 1549 | C | GLN | 1085 | 23.820 | 19.456 | 15.938 | 1.00 | 26.72 |
| ATOM | 1550 | O | GLN | 1085 | 22.698 | 19.736 | 15.557 | 1.00 | 28.76 |
| ATOM | 1551 | N | SER | 1086 | 24.067 | 18.513 | 16.842 | 1.00 | 25.66 |
| ATOM | 1552 | CA | SER | 1086 | 22.919 | 17.873 | 17.483 | 1.00 | 25.15 |
| ATOM | 1553 | CB | SER | 1086 | 23.184 | 16.801 | 18.455 | 1.00 | 25.02 |
| ATOM | 1554 | OG | SER | 1086 | 24.047 | 15.845 | 17.919 | 1.00 | 30.26 |
| ATOM | 1555 | C | SER | 1086 | 22.386 | 18.950 | 18.372 | 1.00 | 25.46 |
| ATOM | 1556 | O | SER | 1086 | 21.192 | 19.233 | 18.327 | 1.00 | 27.32 |
| ATOM | 1557 | N | ASP | 1087 | 23.221 | 19.532 | 19.259 | 1.00 | 23.21 |
| ATOM | 1558 | CA | ASP | 1087 | 22.743 | 20.641 | 20.110 | 1.00 | 18.41 |
| ATOM | 1559 | CB | ASP | 1087 | 23.923 | 21.354 | 20.812 | 1.00 | 17.71 |
| ATOM | 1560 | CG | ASP | 1087 | 24.561 | 20.522 | 21.956 | 1.00 | 16.65 |
| ATOM | 1561 | OD1 | ASP | 1087 | 25.742 | 20.671 | 22.274 | 1.00 | 15.69 |
| ATOM | 1562 | OD2 | ASP | 1087 | 23.860 | 19.750 | 22.574 | 1.00 | 14.37 |
| ATOM | 1563 | C | ASP | 1087 | 21.960 | 21.646 | 19.297 | 1.00 | 15.77 |
| ATOM | 1564 | O | ASP | 1087 | 20.945 | 22.038 | 19.851 | 1.00 | 17.51 |
| ATOM | 1565 | N | VAL | 1088 | 22.265 | 22.007 | 18.024 | 1.00 | 14.83 |
| ATOM | 1566 | CA | VAL | 1088 | 21.433 | 22.982 | 17.236 | 1.00 | 13.42 |
| ATOM | 1567 | CB | VAL | 1088 | 22.043 | 23.297 | 15.884 | 1.00 | 12.77 |
| ATOM | 1568 | CG1 | VAL | 1088 | 21.166 | 24.295 | 15.201 | 1.00 | 13.20 |
| ATOM | 1569 | CG2 | VAL | 1088 | 23.460 | 23.904 | 16.019 | 1.00 | 5.18 |
| ATOM | 1570 | C | VAL | 1088 | 20.067 | 22.370 | 17.033 | 1.00 | 14.86 |
| ATOM | 1571 | O | VAL | 1088 | 19.110 | 23.116 | 17.102 | 1.00 | 19.88 |
| ATOM | 1572 | N | TRP | 1089 | 19.856 | 21.055 | 16.787 | 1.00 | 16.34 |
| ATOM | 1573 | CA | TRP | 1089 | 18.487 | 20.407 | 16.734 | 1.00 | 13.67 |
| ATOM | 1574 | CB | TRP | 1089 | 18.643 | 18.837 | 16.667 | 1.00 | 12.79 |
| ATOM | 1575 | CG | TRP | 1089 | 17.347 | 18.112 | 16.532 | 1.00 | 13.47 |
| ATOM | 1576 | CD2 | TRP | 1089 | 16.712 | 17.664 | 15.311 | 1.00 | 12.90 |
| ATOM | 1577 | CE2 | TRP | 1089 | 15.353 | 17.383 | 15.615 | 1.00 | 16.02 |
| ATOM | 1578 | CE3 | TRP | 1089 | 17.167 | 17.482 | 13.982 | 1.00 | 14.82 |
| ATOM | 1579 | CD1 | TRP | 1089 | 16.340 | 18.056 | 17.517 | 1.00 | 15.04 |
| ATOM | 1580 | NE1 | TRP | 1089 | 15.144 | 17.627 | 16.975 | 1.00 | 18.31 |
| ATOM | 1581 | CZ2 | TRP | 1089 | 14.488 | 16.940 | 14.599 | 1.00 | 13.72 |
| ATOM | 1582 | CZ3 | TRP | 1089 | 16.290 | 17.037 | 12.992 | 1.00 | 14.99 |
| ATOM | 1583 | CH2 | TRP | 1089 | 14.956 | 16.774 | 13.297 | 1.00 | 16.22 |
| ATOM | 1584 | C | TRP | 1089 | 17.717 | 20.771 | 18.047 | 1.00 | 16.06 |
| ATOM | 1585 | O | TRP | 1089 | 16.695 | 21.460 | 18.047 | 1.00 | 17.98 |
| ATOM | 1586 | N | SER | 1090 | 18.262 | 20.397 | 19.247 | 1.00 | 17.49 |
| ATOM | 1587 | CA | SER | 1090 | 17.691 | 20.642 | 20.632 | 1.00 | 15.54 |
| ATOM | 1588 | CB | SER | 1090 | 18.676 | 20.245 | 21.732 | 1.00 | 15.35 |
| ATOM | 1589 | OG | SER | 1090 | 19.095 | 18.912 | 21.523 | 1.00 | 17.99 |
| ATOM | 1590 | C | SER | 1090 | 17.360 | 22.089 | 20.872 | 1.00 | 13.35 |
| ATOM | 1591 | O | SER | 1090 | 16.468 | 22.455 | 21.634 | 1.00 | 14.62 |
| ATOM | 1592 | N | PHE | 1091 | 18.177 | 22.960 | 20.311 | 1.00 | 15.03 |
| ATOM | 1593 | CA | PHE | 1091 | 18.030 | 24.445 | 20.362 | 1.00 | 15.75 |
| ATOM | 1594 | CB | PHE | 1091 | 19.332 | 25.084 | 19.829 | 1.00 | 17.20 |
| ATOM | 1595 | CG | PHE | 1091 | 19.146 | 26.547 | 19.897 | 1.00 | 21.06 |
| ATOM | 1596 | CD1 | PHE | 1091 | 19.273 | 27.223 | 21.112 | 1.00 | 18.67 |
| ATOM | 1597 | CD2 | PHE | 1091 | 18.770 | 27.274 | 18.729 | 1.00 | 19.43 |
| ATOM | 1598 | CE1 | PHE | 1091 | 19.020 | 28.603 | 21.151 | 1.00 | 16.31 |
| ATOM | 1599 | CE2 | PHE | 1091 | 18.523 | 28.643 | 18.791 | 1.00 | 13.37 |
| ATOM | 1600 | CZ | PHE | 1091 | 18.651 | 29.305 | 20.009 | 1.00 | 13.81 |
| ATOM | 1601 | C | PHE | 1091 | 16.800 | 24.745 | 19.488 | 1.00 | 16.90 |
| ATOM | 1602 | O | PHE | 1091 | 15.918 | 25.491 | 19.918 | 1.00 | 19.37 |
| ATOM | 1603 | N | GLY | 1092 | 16.643 | 24.120 | 18.304 | 1.00 | 14.11 |
| ATOM | 1604 | CA | GLY | 1092 | 15.426 | 24.345 | 17.509 | 1.00 | 10.42 |
| ATOM | 1605 | C | GLY | 1092 | 14.265 | 23.911 | 18.372 | 1.00 | 9.12 |
| ATOM | 1606 | O | GLY | 1092 | 13.244 | 24.573 | 18.457 | 1.00 | 12.56 |
| ATOM | 1607 | N | VAL | 1093 | 14.314 | 22.786 | 19.050 | 1.00 | 7.97 |
| ATOM | 1608 | CA | VAL | 1093 | 13.151 | 22.452 | 19.920 | 1.00 | 7.51 |
| ATOM | 1609 | CB | VAL | 1093 | 13.240 | 21.064 | 20.587 | 1.00 | 7.73 |
| ATOM | 1610 | CG1 | VAL | 1093 | 11.841 | 20.814 | 21.149 | 1.00 | 6.52 |
| ATOM | 1611 | CG2 | VAL | 1093 | 13.609 | 19.922 | 19.653 | 1.00 | 8.95 |
| ATOM | 1612 | C | VAL | 1093 | 13.035 | 23.471 | 21.075 | 1.00 | 10.99 |
| ATOM | 1613 | O | VAL | 1093 | 11.929 | 23.859 | 21.488 | 1.00 | 14.40 |
| ATOM | 1614 | N | LEU | 1094 | 14.167 | 23.888 | 21.682 | 1.00 | 9.45 |
| ATOM | 1615 | CA | LEU | 1094 | 14.154 | 24.882 | 22.781 | 1.00 | 10.47 |
| ATOM | 1616 | CB | LEU | 1094 | 15.596 | 25.163 | 23.165 | 1.00 | 11.81 |
| ATOM | 1617 | CG | LEU | 1094 | 15.969 | 25.530 | 24.616 | 1.00 | 14.94 |
| ATOM | 1618 | CD1 | LEU | 1094 | 17.407 | 25.917 | 24.477 | 1.00 | 16.24 |
| ATOM | 1619 | CD2 | LEU | 1094 | 15.293 | 26.716 | 25.263 | 1.00 | 12.17 |
| ATOM | 1620 | C | LEU | 1094 | 13.425 | 26.171 | 22.329 | 1.00 | 13.22 |
| ATOM | 1621 | O | LEU | 1094 | 12.698 | 26.791 | 23.121 | 1.00 | 15.76 |
| ATOM | 1622 | N | LEU | 1095 | 13.586 | 26.615 | 21.048 | 1.00 | 12.60 |
| ATOM | 1623 | CA | LEU | 1095 | 12.854 | 27.794 | 20.445 | 1.00 | 8.78 |
| ATOM | 1624 | CB | LEU | 1095 | 13.347 | 28.109 | 19.071 | 1.00 | 8.20 |
| ATOM | 1625 | CG | LEU | 1095 | 14.697 | 28.744 | 19.140 | 1.00 | 4.85 |
| ATOM | 1626 | CD1 | LEU | 1095 | 15.163 | 29.054 | 17.754 | 1.00 | 5.92 |
| ATOM | 1627 | CD2 | LEU | 1095 | 14.602 | 30.082 | 19.804 | 1.00 | 3.71 |
| ATOM | 1628 | C | LEU | 1095 | 11.375 | 27.465 | 20.337 | 1.00 | 9.96 |
| ATOM | 1629 | O | LEU | 1095 | 10.511 | 28.320 | 20.573 | 1.00 | 14.20 |
| ATOM | 1630 | N | TRP | 1096 | 10.991 | 26.237 | 19.993 | 1.00 | 9.08 |
| ATOM | 1631 | CA | TRP | 1096 | 9.547 | 25.871 | 19.964 | 1.00 | 8.83 |
| ATOM | 1632 | CB | TRP | 1096 | 9.389 | 24.382 | 19.657 | 1.00 | 8.41 |
| ATOM | 1633 | CG | TRP | 1096 | 7.969 | 23.992 | 19.466 | 1.00 | 11.29 |
| ATOM | 1634 | CD2 | TRP | 1096 | 6.949 | 23.579 | 20.389 | 1.00 | 9.06 |
| ATOM | 1635 | CE2 | TRP | 1096 | 5.748 | 23.430 | 19.662 | 1.00 | 6.19 |
| ATOM | 1636 | CE3 | TRP | 1096 | 6.931 | 23.346 | 21.758 | 1.00 | 9.71 |
| ATOM | 1637 | CD1 | TRP | 1096 | 7.300 | 24.051 | 18.224 | 1.00 | 13.67 |
| ATOM | 1638 | NE1 | TRP | 1096 | 5.978 | 23.714 | 18.358 | 1.00 | 12.75 |
| ATOM | 1639 | CZ2 | TRP | 1096 | 4.589 | 23.045 | 20.293 | 1.00 | 8.09 |
| ATOM | 1640 | CZ3 | TRP | 1096 | 5.731 | 22.961 | 22.380 | 1.00 | 8.82 |
| ATOM | 1641 | CH2 | TRP | 1096 | 4.564 | 22.814 | 21.662 | 1.00 | 8.55 |
| ATOM | 1642 | C | TRP | 1096 | 8.963 | 26.147 | 21.347 | 1.00 | 12.22 |
| ATOM | 1643 | O | TRP | 1096 | 7.952 | 26.827 | 21.515 | 1.00 | 17.97 |
| ATOM | 1644 | N | GLU | 1097 | 9.564 | 25.548 | 22.399 | 1.00 | 17.09 |
| ATOM | 1645 | CA | GLU | 1097 | 9.205 | 25.686 | 23.874 | 1.00 | 15.70 |
| ATOM | 1646 | CB | GLU | 1097 | 10.313 | 25.004 | 24.757 | 1.00 | 15.25 |
| ATOM | 1647 | CG | GLU | 1097 | 10.468 | 23.459 | 24.828 | 1.00 | 16.11 |
| ATOM | 1648 | CD | GLU | 1097 | 11.425 | 23.035 | 25.947 | 1.00 | 18.88 |
| ATOM | 1649 | OE1 | GLU | 1097 | 10.960 | 22.633 | 27.023 | 1.00 | 25.86 |
| ATOM | 1650 | OE2 | GLU | 1097 | 12.633 | 23.102 | 25.751 | 1.00 | 15.17 |
| ATOM | 1651 | C | GLU | 1097 | 9.071 | 27.187 | 24.340 | 1.00 | 14.75 |
| ATOM | 1652 | O | GLU | 1097 | 8.165 | 27.624 | 25.062 | 1.00 | 13.02 |
| ATOM | 1653 | N | ILE | 1098 | 10.081 | 28.007 | 24.028 | 1.00 | 12.83 |
| ATOM | 1654 | CA | ILE | 1098 | 10.004 | 29.435 | 24.326 | 1.00 | 9.33 |
| ATOM | 1655 | CB | ILE | 1098 | 11.278 | 30.082 | 23.837 | 1.00 | 11.58 |
| ATOM | 1656 | CG2 | ILE | 1098 | 11.074 | 31.590 | 23.919 | 1.00 | 17.93 |
| ATOM | 1657 | CG1 | ILE | 1098 | 12.482 | 29.702 | 24.672 | 1.00 | 14.18 |
| ATOM | 1658 | CD1 | ILE | 1098 | 13.757 | 30.435 | 24.263 | 1.00 | 14.72 |
| ATOM | 1659 | C | ILE | 1098 | 8.799 | 30.042 | 23.595 | 1.00 | 10.94 |
| ATOM | 1660 | O | ILE | 1098 | 7.866 | 30.485 | 24.256 | 1.00 | 12.04 |
| ATOM | 1661 | N | PHE | 1099 | 8.693 | 30.041 | 22.232 | 1.00 | 12.92 |
| ATOM | 1662 | CA | PHE | 1099 | 7.507 | 30.674 | 21.532 | 1.00 | 12.03 |
| ATOM | 1663 | CB | PHE | 1099 | 7.859 | 30.932 | 20.040 | 1.00 | 12.44 |
| ATOM | 1664 | CG | PHE | 1099 | 9.014 | 31.834 | 19.955 | 1.00 | 8.48 |
| ATOM | 1665 | CD1 | PHE | 1099 | 10.305 | 31.350 | 20.089 | 1.00 | 3.92 |
| ATOM | 1666 | CD2 | PHE | 1099 | 8.766 | 33.167 | 19.850 | 1.00 | 6.57 |
| ATOM | 1667 | CE1 | PHE | 1099 | 11.375 | 32.252 | 20.136 | 1.00 | 4.60 |
| ATOM | 1668 | CE2 | PHE | 1099 | 9.820 | 34.074 | 19.884 | 1.00 | 10.18 |
| ATOM | 1669 | CZ | PHE | 1099 | 11.137 | 33.619 | 20.032 | 1.00 | 9.99 |
| ATOM | 1670 | C | PHE | 1099 | 6.112 | 30.002 | 21.602 | 1.00 | 13.90 |
| ATOM | 1671 | O | PHE | 1099 | 5.092 | 30.342 | 20.976 | 1.00 | 11.94 |
| ATOM | 1672 | N | SER | 1100 | 6.062 | 28.919 | 22.325 | 1.00 | 16.29 |
| ATOM | 1673 | CA | SER | 1100 | 4.808 | 28.194 | 22.609 | 1.00 | 16.71 |
| ATOM | 1674 | CB | SER | 1100 | 5.007 | 26.672 | 22.424 | 1.00 | 16.33 |
| ATOM | 1675 | OG | SER | 1100 | 5.824 | 26.242 | 23.478 | 1.00 | 19.32 |
| ATOM | 1676 | C | SER | 1100 | 4.504 | 28.515 | 24.081 | 1.00 | 15.92 |
| ATOM | 1677 | O | SER | 1100 | 3.437 | 28.212 | 24.611 | 1.00 | 17.01 |
| ATOM | 1678 | N | LEU | 1101 | 5.429 | 29.276 | 24.697 | 1.00 | 15.51 |
| ATOM | 1679 | CA | LEU | 1101 | 5.465 | 29.716 | 26.089 | 1.00 | 12.92 |
| ATOM | 1680 | CB | LEU | 1101 | 4.355 | 30.699 | 26.458 | 1.00 | 8.06 |
| ATOM | 1681 | CG | LEU | 1101 | 4.533 | 32.159 | 25.975 | 1.00 | 5.55 |
| ATOM | 1682 | CD1 | LEU | 1101 | 3.458 | 33.061 | 26.605 | 1.00 | 3.18 |
| ATOM | 1683 | CD2 | LEU | 1101 | 5.882 | 32.654 | 26.345 | 1.00 | 3.84 |
| ATOM | 1684 | C | LEU | 1101 | 5.369 | 28.576 | 27.107 | 1.00 | 15.40 |
| ATOM | 1685 | O | LEU | 1101 | 4.431 | 28.526 | 27.890 | 1.00 | 14.78 |
| ATOM | 1686 | N | GLY | 1102 | 6.357 | 27.669 | 27.144 | 1.00 | 15.65 |
| ATOM | 1687 | CA | GLY | 1102 | 6.380 | 26.564 | 28.124 | 1.00 | 14.84 |
| ATOM | 1688 | C | GLY | 1102 | 5.626 | 25.311 | 27.790 | 1.00 | 12.52 |
| ATOM | 1689 | O | GLY | 1102 | 5.396 | 24.451 | 28.619 | 1.00 | 15.72 |
| ATOM | 1690 | N | ALA | 1103 | 5.166 | 25.148 | 26.598 | 1.00 | 14.40 |
| ATOM | 1691 | CA | ALA | 1103 | 4.457 | 23.902 | 26.277 | 1.00 | 15.17 |
| ATOM | 1692 | CB | ALA | 1103 | 3.600 | 24.015 | 24.985 | 1.00 | 16.74 |
| ATOM | 1693 | C | ALA | 1103 | 5.399 | 22.731 | 26.071 | 1.00 | 12.39 |
| ATOM | 1694 | O | ALA | 1103 | 6.584 | 22.884 | 25.896 | 1.00 | 12.20 |
| ATOM | 1695 | N | SER | 1104 | 4.923 | 21.519 | 26.193 | 1.00 | 13.14 |
| ATOM | 1696 | CA | SER | 1104 | 5.749 | 20.332 | 25.943 | 1.00 | 14.28 |
| ATOM | 1697 | CB | SER | 1104 | 5.055 | 19.140 | 26.646 | 1.00 | 15.28 |
| ATOM | 1698 | OG | SER | 1104 | 5.976 | 18.069 | 26.897 | 1.00 | 20.88 |
| ATOM | 1699 | C | SER | 1104 | 5.852 | 20.118 | 24.396 | 1.00 | 17.06 |
| ATOM | 1700 | O | SER | 1104 | 4.875 | 20.261 | 23.611 | 1.00 | 13.19 |
| ATOM | 1701 | N | PRO | 1105 | 7.057 | 19.854 | 23.864 | 1.00 | 16.34 |
| ATOM | 1702 | CD | PRO | 1105 | 8.387 | 20.171 | 24.385 | 1.00 | 15.63 |
| ATOM | 1703 | CA | PRO | 1105 | 7.137 | 19.468 | 22.465 | 1.00 | 17.11 |
| ATOM | 1704 | CB | PRO | 1105 | 8.604 | 19.072 | 22.326 | 1.00 | 13.02 |
| ATOM | 1705 | CG | PRO | 1105 | 9.209 | 20.205 | 23.126 | 1.00 | 10.15 |
| ATOM | 1706 | C | PRO | 1105 | 6.121 | 18.398 | 22.053 | 1.00 | 19.27 |
| ATOM | 1707 | O | PRO | 1105 | 5.475 | 17.766 | 22.869 | 1.00 | 23.30 |
| ATOM | 1708 | N | TYR | 1106 | 5.986 | 18.187 | 20.742 | 1.00 | 22.83 |
| ATOM | 1709 | CA | TYR | 1106 | 5.142 | 17.203 | 19.986 | 1.00 | 19.50 |
| ATOM | 1710 | CB | TYR | 1106 | 5.963 | 15.959 | 19.732 | 1.00 | 12.64 |
| ATOM | 1711 | CG | TYR | 1106 | 7.278 | 16.227 | 19.109 | 1.00 | 11.95 |
| ATOM | 1712 | CD1 | TYR | 1106 | 8.460 | 16.208 | 19.872 | 1.00 | 13.48 |
| ATOM | 1713 | CE1 | TYR | 1106 | 9.680 | 16.537 | 19.253 | 1.00 | 16.37 |
| ATOM | 1714 | CD2 | TYR | 1106 | 7.318 | 16.550 | 17.749 | 1.00 | 11.65 |
| ATOM | 1715 | CE2 | TYR | 1106 | 8.516 | 16.867 | 17.127 | 1.00 | 14.83 |
| ATOM | 1716 | CZ | TYR | 1106 | 9.727 | 16.880 | 17.871 | 1.00 | 19.04 |
| ATOM | 1717 | OH | TYR | 1106 | 10.908 | 17.348 | 17.228 | 1.00 | 17.89 |
| ATOM | 1718 | C | TYR | 1106 | 3.808 | 16.843 | 20.585 | 1.00 | 21.40 |
| ATOM | 1719 | O | TYR | 1106 | 3.495 | 15.700 | 20.868 | 1.00 | 23.90 |
| ATOM | 1720 | N | PRO | 1107 | 2.964 | 17.863 | 20.721 | 1.00 | 22.07 |
| ATOM | 1721 | CD | PRO | 1107 | 3.242 | 19.275 | 20.285 | 1.00 | 24.51 |
| ATOM | 1722 | CA | PRO | 1107 | 1.603 | 17.694 | 21.248 | 1.00 | 24.21 |
| ATOM | 1723 | CB | PRO | 1107 | 0.984 | 19.108 | 21.059 | 1.00 | 24.40 |
| ATOM | 1724 | CG | PRO | 1107 | 1.855 | 19.903 | 20.054 | 1.00 | 22.46 |
| ATOM | 1725 | C | PRO | 1107 | 0.812 | 16.573 | 20.588 | 1.00 | 26.77 |
| ATOM | 1726 | O | PRO | 1107 | 0.706 | 16.481 | 19.357 | 1.00 | 26.38 |
| ATOM | 1727 | N | GLY | 1108 | 0.140 | 15.749 | 21.361 | 1.00 | 31.02 |
| ATOM | 1728 | CA | GLY | 1108 | −0.633 | 14.613 | 20.766 | 1.00 | 38.57 |
| ATOM | 1729 | C | GLY | 1108 | 0.235 | 13.348 | 20.559 | 1.00 | 42.74 |
| ATOM | 1730 | O | GLY | 1108 | 0.002 | 12.319 | 21.204 | 1.00 | 47.64 |
| ATOM | 1731 | N | VAL | 1109 | 1.253 | 13.450 | 19.687 | 1.00 | 41.15 |
| ATOM | 1732 | CA | VAL | 1109 | 2.242 | 12.400 | 19.349 | 1.00 | 39.04 |
| ATOM | 1733 | CB | VAL | 1109 | 3.423 | 13.072 | 18.576 | 1.00 | 40.45 |
| ATOM | 1734 | CG1 | VAL | 1109 | 4.593 | 12.079 | 18.411 | 1.00 | 42.61 |
| ATOM | 1735 | CG2 | VAL | 1109 | 2.957 | 13.568 | 17.195 | 1.00 | 42.46 |
| ATOM | 1736 | C | VAL | 1109 | 2.818 | 11.630 | 20.552 | 1.00 | 37.34 |
| ATOM | 1737 | O | VAL | 1109 | 3.228 | 12.188 | 21.562 | 1.00 | 38.28 |
| ATOM | 1738 | N | LYS | 1110 | 2.785 | 10.296 | 20.456 | 1.00 | 36.51 |
| ATOM | 1739 | CA | LYS | 1110 | 3.321 | 9.368 | 21.464 | 1.00 | 33.65 |
| ATOM | 1740 | CB | LYS | 1110 | 2.593 | 8.000 | 21.469 | 1.00 | 35.67 |
| ATOM | 1741 | C | LYS | 1110 | 4.743 | 9.135 | 21.020 | 1.00 | 32.90 |
| ATOM | 1742 | O | LYS | 1110 | 5.045 | 8.941 | 19.837 | 1.00 | 28.20 |
| ATOM | 1743 | N | ILE | 1111 | 5.654 | 9.163 | 21.963 | 1.00 | 34.79 |
| ATOM | 1744 | CA | ILE | 1111 | 7.072 | 9.002 | 21.627 | 1.00 | 34.96 |
| ATOM | 1745 | CB | ILE | 1111 | 7.894 | 9.828 | 22.686 | 1.00 | 29.93 |
| ATOM | 1746 | CG2 | ILE | 1111 | 9.374 | 9.943 | 22.282 | 1.00 | 27.52 |
| ATOM | 1747 | CG1 | ILE | 1111 | 7.280 | 11.235 | 22.791 | 1.00 | 24.64 |
| ATOM | 1748 | CD1 | ILE | 1111 | 7.249 | 12.051 | 21.493 | 1.00 | 22.47 |
| ATOM | 1749 | C | ILE | 1111 | 7.356 | 7.511 | 21.579 | 1.00 | 38.19 |
| ATOM | 1750 | O | ILE | 1111 | 7.722 | 6.844 | 22.544 | 1.00 | 39.52 |
| ATOM | 1751 | N | ASP | 1112 | 6.979 | 7.005 | 20.398 | 1.00 | 42.08 |
| ATOM | 1752 | CA | ASP | 1112 | 7.060 | 5.581 | 19.946 | 1.00 | 44.34 |
| ATOM | 1753 | CB | ASP | 1112 | 5.561 | 5.060 | 19.620 | 1.00 | 45.09 |
| ATOM | 1754 | CG | ASP | 1112 | 4.738 | 5.594 | 18.411 | 1.00 | 45.93 |
| ATOM | 1755 | OD1 | ASP | 1112 | 3.574 | 5.240 | 18.295 | 1.00 | 46.58 |
| ATOM | 1756 | OD2 | ASP | 1112 | 5.215 | 6.342 | 17.577 | 1.00 | 47.21 |
| ATOM | 1757 | C | ASP | 1112 | 7.994 | 5.318 | 18.752 | 1.00 | 45.81 |
| ATOM | 1758 | O | ASP | 1112 | 8.817 | 6.170 | 18.385 | 1.00 | 44.87 |
| ATOM | 1759 | N | GLU | 1113 | 7.929 | 4.102 | 18.157 | 1.00 | 49.66 |
| ATOM | 1760 | CA | GLU | 1113 | 8.742 | 3.729 | 16.942 | 1.00 | 51.81 |
| ATOM | 1761 | CB | GLU | 1113 | 8.531 | 2.217 | 16.554 | 1.00 | 54.69 |
| ATOM | 1762 | C | GLU | 1113 | 8.321 | 4.610 | 15.723 | 1.00 | 51.21 |
| ATOM | 1763 | O | GLU | 1113 | 9.107 | 5.002 | 14.851 | 1.00 | 48.46 |
| ATOM | 1764 | N | GLU | 1114 | 7.022 | 4.979 | 15.685 | 1.00 | 50.44 |
| ATOM | 1765 | CA | GLU | 1114 | 6.555 | 5.845 | 14.600 | 1.00 | 49.42 |
| ATOM | 1766 | CB | GLU | 1114 | 4.983 | 5.796 | 14.502 | 1.00 | 54.53 |
| ATOM | 1767 | CG | GLU | 1114 | 4.382 | 4.376 | 14.366 | 1.00 | 62.16 |
| ATOM | 1768 | CD | GLU | 1114 | 5.036 | 3.349 | 13.387 | 1.00 | 68.41 |
| ATOM | 1769 | OE1 | GLU | 1114 | 6.196 | 3.407 | 12.951 | 1.00 | 69.59 |
| ATOM | 1770 | OE2 | GLU | 1114 | 4.347 | 2.375 | 13.096 | 1.00 | 72.80 |
| ATOM | 1771 | C | GLU | 1114 | 7.083 | 7.254 | 14.781 | 1.00 | 45.44 |
| ATOM | 1772 | O | GLU | 1114 | 7.685 | 7.786 | 13.875 | 1.00 | 43.12 |
| ATOM | 1773 | N | PHE | 1115 | 7.033 | 7.857 | 15.948 | 1.00 | 42.27 |
| ATOM | 1774 | CA | PHE | 1115 | 7.557 | 9.230 | 16.128 | 1.00 | 40.17 |
| ATOM | 1775 | CB | PHE | 1115 | 7.552 | 9.544 | 17.657 | 1.00 | 37.46 |
| ATOM | 1776 | CG | PHE | 1115 | 8.535 | 10.577 | 18.048 | 1.00 | 34.89 |
| ATOM | 1777 | CD1 | PHE | 1115 | 9.855 | 10.208 | 18.342 | 1.00 | 32.95 |
| ATOM | 1778 | CD2 | PHE | 1115 | 8.168 | 11.926 | 18.076 | 1.00 | 34.45 |
| ATOM | 1779 | CE1 | PHE | 1115 | 10.824 | 11.162 | 18.650 | 1.00 | 35.34 |
| ATOM | 1780 | CE2 | PHE | 1115 | 9.142 | 12.883 | 18.391 | 1.00 | 34.78 |
| ATOM | 1781 | CZ | PHE | 1115 | 10.461 | 12.512 | 18.673 | 1.00 | 31.65 |
| ATOM | 1782 | C | PHE | 1115 | 8.937 | 9.278 | 15.538 | 1.00 | 38.95 |
| ATOM | 1783 | O | PHE | 1115 | 9.281 | 10.110 | 14.707 | 1.00 | 40.23 |
| ATOM | 1784 | N | CYS | 1116 | 9.748 | 8.322 | 15.960 | 1.00 | 40.57 |
| ATOM | 1785 | CA | CYS | 1116 | 11.122 | 8.213 | 15.463 | 1.00 | 40.53 |
| ATOM | 1786 | CB | CYS | 1116 | 11.865 | 7.124 | 16.246 | 1.00 | 40.94 |
| ATOM | 1787 | SG | CYS | 1116 | 12.179 | 7.314 | 18.030 | 1.00 | 42.44 |
| ATOM | 1788 | C | CYS | 1116 | 11.095 | 7.882 | 13.960 | 1.00 | 42.50 |
| ATOM | 1789 | O | CYS | 1116 | 11.926 | 8.409 | 13.227 | 1.00 | 42.47 |
| ATOM | 1790 | N | ARG | 1117 | 10.206 | 7.009 | 13.441 | 1.00 | 43.56 |
| ATOM | 1791 | CA | ARG | 1117 | 10.125 | 6.721 | 11.966 | 1.00 | 46.24 |
| ATOM | 1792 | CB | ARG | 1117 | 8.991 | 5.697 | 11.683 | 1.00 | 48.27 |
| ATOM | 1793 | CG | ARG | 1117 | 8.629 | 5.179 | 10.263 | 1.00 | 49.86 |
| ATOM | 1794 | CD | ARG | 1117 | 7.140 | 4.727 | 10.237 | 1.00 | 47.87 |
| ATOM | 1795 | NE | ARG | 1117 | 6.293 | 5.810 | 9.688 | 1.00 | 50.64 |
| ATOM | 1796 | CZ | ARG | 1117 | 4.994 | 6.039 | 9.990 | 1.00 | 52.53 |
| ATOM | 1797 | NH1 | ARG | 1117 | 4.388 | 7.053 | 9.383 | 1.00 | 55.06 |
| ATOM | 1798 | NH2 | ARG | 1117 | 4.261 | 5.357 | 10.874 | 1.00 | 50.82 |
| ATOM | 1799 | C | ARG | 1117 | 9.809 | 8.051 | 11.251 | 1.00 | 46.33 |
| ATOM | 1800 | O | ARG | 1117 | 10.540 | 8.540 | 10.392 | 1.00 | 50.20 |
| ATOM | 1801 | N | ARG | 1118 | 8.722 | 8.721 | 11.586 | 1.00 | 45.44 |
| ATOM | 1802 | CA | ARG | 1118 | 8.392 | 10.025 | 10.990 | 1.00 | 43.71 |
| ATOM | 1803 | CB | ARG | 1118 | 7.121 | 10.542 | 11.664 | 1.00 | 47.21 |
| ATOM | 1804 | CG | ARG | 1118 | 5.903 | 9.620 | 11.471 | 1.00 | 50.07 |
| ATOM | 1805 | CD | ARG | 1118 | 5.270 | 9.192 | 12.819 | 1.00 | 54.40 |
| ATOM | 1806 | NE | ARG | 1118 | 4.341 | 10.205 | 13.352 | 1.00 | 59.63 |
| ATOM | 1807 | CZ | ARG | 1118 | 3.629 | 10.091 | 14.510 | 1.00 | 61.49 |
| ATOM | 1808 | NH1 | ARG | 1118 | 2.817 | 11.115 | 14.815 | 1.00 | 63.73 |
| ATOM | 1809 | NH2 | ARG | 1118 | 3.709 | 9.067 | 15.387 | 1.00 | 59.39 |
| ATOM | 1810 | C | ARG | 1118 | 9.517 | 11.093 | 11.075 | 1.00 | 42.51 |
| ATOM | 1811 | O | ARG | 1118 | 9.574 | 11.938 | 10.197 | 1.00 | 43.92 |
| ATOM | 1812 | N | LEU | 1119 | 10.409 | 11.232 | 12.065 | 1.00 | 40.29 |
| ATOM | 1813 | CA | LEU | 1119 | 11.454 | 12.298 | 11.945 | 1.00 | 39.16 |
| ATOM | 1814 | CB | LEU | 1119 | 12.254 | 12.491 | 13.208 | 1.00 | 38.62 |
| ATOM | 1815 | CG | LEU | 1119 | 11.584 | 12.776 | 14.538 | 1.00 | 37.73 |
| ATOM | 1816 | CD1 | LEU | 1119 | 12.813 | 12.596 | 15.471 | 1.00 | 40.39 |
| ATOM | 1817 | CD2 | LEU | 1119 | 10.804 | 14.131 | 14.675 | 1.00 | 35.94 |
| ATOM | 1818 | C | LEU | 1119 | 12.528 | 12.087 | 10.848 | 1.00 | 41.21 |
| ATOM | 1819 | O | LEU | 1119 | 13.243 | 13.022 | 10.441 | 1.00 | 39.66 |
| ATOM | 1820 | N | LYS | 1120 | 12.790 | 10.841 | 10.399 | 1.00 | 43.68 |
| ATOM | 1821 | CA | LYS | 1120 | 13.802 | 10.652 | 9.314 | 1.00 | 45.61 |
| ATOM | 1822 | CB | LYS | 1120 | 14.172 | 9.161 | 9.104 | 1.00 | 48.57 |
| ATOM | 1823 | CG | LYS | 1120 | 14.831 | 8.479 | 10.332 | 1.00 | 52.47 |
| ATOM | 1824 | CD | LYS | 1120 | 16.081 | 7.537 | 10.152 | 1.00 | 54.03 |
| ATOM | 1825 | CE | LYS | 1120 | 17.375 | 8.346 | 9.854 | 1.00 | 56.83 |
| ATOM | 1826 | NZ | LYS | 1120 | 18.541 | 7.890 | 10.645 | 1.00 | 56.71 |
| ATOM | 1827 | C | LYS | 1120 | 13.190 | 11.200 | 8.021 | 1.00 | 46.83 |
| ATOM | 1828 | O | LYS | 1120 | 13.882 | 11.754 | 7.158 | 1.00 | 46.28 |
| ATOM | 1829 | N | GLU | 1121 | 11.845 | 11.010 | 7.943 | 1.00 | 47.44 |
| ATOM | 1830 | CA | GLU | 1121 | 10.969 | 11.484 | 6.845 | 1.00 | 48.97 |
| ATOM | 1831 | CB | GLU | 1121 | 9.437 | 11.409 | 7.112 | 1.00 | 51.08 |
| ATOM | 1832 | CG | GLU | 1121 | 8.429 | 10.296 | 6.808 | 1.00 | 53.50 |
| ATOM | 1833 | CD | GLU | 1121 | 8.746 | 9.028 | 7.557 | 1.00 | 54.89 |
| ATOM | 1834 | OE1 | GLU | 1121 | 7.785 | 8.384 | 7.985 | 1.00 | 58.40 |
| ATOM | 1835 | OE2 | GLU | 1121 | 9.924 | 8.671 | 7.672 | 1.00 | 54.55 |
| ATOM | 1836 | C | GLU | 1121 | 11.127 | 12.975 | 6.621 | 1.00 | 49.03 |
| ATOM | 1837 | O | GLU | 1121 | 11.263 | 13.491 | 5.509 | 1.00 | 52.96 |
| ATOM | 1838 | N | GLY | 1122 | 10.963 | 13.674 | 7.742 | 1.00 | 46.68 |
| ATOM | 1839 | CA | GLY | 1122 | 10.979 | 15.139 | 7.843 | 1.00 | 43.47 |
| ATOM | 1840 | C | GLY | 1122 | 9.681 | 15.592 | 8.549 | 1.00 | 41.28 |
| ATOM | 1841 | O | GLY | 1122 | 9.111 | 16.624 | 8.220 | 1.00 | 41.39 |
| ATOM | 1842 | N | THR | 1123 | 9.119 | 14.810 | 9.483 | 1.00 | 39.99 |
| ATOM | 1843 | CA | THR | 1123 | 7.910 | 15.242 | 10.226 | 1.00 | 38.69 |
| ATOM | 1844 | CB | THR | 1123 | 7.230 | 14.057 | 11.003 | 1.00 | 38.73 |
| ATOM | 1845 | OG1 | THR | 1123 | 6.338 | 13.450 | 10.097 | 1.00 | 42.71 |
| ATOM | 1846 | CG2 | THR | 1123 | 6.338 | 14.413 | 12.197 | 1.00 | 40.39 |
| ATOM | 1847 | C | THR | 1123 | 8.353 | 16.289 | 11.232 | 1.00 | 37.06 |
| ATOM | 1848 | O | THR | 1123 | 9.180 | 16.007 | 12.086 | 1.00 | 37.08 |
| ATOM | 1849 | N | ARG | 1124 | 8.013 | 17.546 | 11.023 | 1.00 | 34.87 |
| ATOM | 1850 | CA | ARG | 1124 | 8.332 | 18.615 | 11.991 | 1.00 | 32.41 |
| ATOM | 1851 | CB | ARG | 1124 | 8.863 | 19.944 | 11.288 | 1.00 | 29.99 |
| ATOM | 1852 | CG | ARG | 1124 | 10.260 | 19.884 | 10.593 | 1.00 | 26.99 |
| ATOM | 1853 | CD | ARG | 1124 | 11.085 | 18.819 | 11.342 | 1.00 | 29.64 |
| ATOM | 1854 | NE | ARG | 1124 | 12.353 | 18.342 | 10.758 | 1.00 | 29.16 |
| ATOM | 1855 | CZ | ARG | 1124 | 12.584 | 17.022 | 10.579 | 1.00 | 28.45 |
| ATOM | 1856 | NH1 | ARG | 1124 | 11.682 | 16.113 | 10.900 | 1.00 | 29.53 |
| ATOM | 1857 | NH2 | ARG | 1124 | 13.743 | 16.579 | 10.115 | 1.00 | 27.84 |
| ATOM | 1858 | C | ARG | 1124 | 7.111 | 19.006 | 12.851 | 1.00 | 33.35 |
| ATOM | 1859 | O | ARG | 1124 | 5.948 | 18.609 | 12.626 | 1.00 | 34.01 |
| ATOM | 1860 | N | MET | 1125 | 7.400 | 19.761 | 13.911 | 1.00 | 31.12 |
| ATOM | 1861 | CA | MET | 1125 | 6.388 | 20.289 | 14.799 | 1.00 | 27.94 |
| ATOM | 1862 | CB | MET | 1125 | 7.067 | 20.846 | 16.095 | 1.00 | 24.62 |
| ATOM | 1863 | CG | MET | 1125 | 6.752 | 19.927 | 17.255 | 1.00 | 14.78 |
| ATOM | 1864 | SD | MET | 1125 | 7.309 | 20.490 | 18.818 | 1.00 | 10.40 |
| ATOM | 1865 | CE | MET | 1125 | 9.082 | 20.497 | 18.646 | 1.00 | 2.00 |
| ATOM | 1866 | C | MET | 1125 | 5.639 | 21.413 | 14.077 | 1.00 | 29.63 |
| ATOM | 1867 | O | MET | 1125 | 6.179 | 22.087 | 13.190 | 1.00 | 29.58 |
| ATOM | 1868 | N | ARG | 1126 | 4.354 | 21.549 | 14.379 | 1.00 | 32.36 |
| ATOM | 1869 | CA | ARG | 1126 | 3.485 | 22.619 | 13.827 | 1.00 | 35.30 |
| ATOM | 1870 | CB | ARG | 1126 | 2.046 | 22.347 | 14.318 | 1.00 | 41.81 |
| ATOM | 1871 | CG | ARG | 1126 | 1.998 | 22.048 | 15.900 | 1.00 | 48.40 |
| ATOM | 1872 | CD | ARG | 1126 | 1.564 | 23.240 | 16.800 | 1.00 | 49.82 |
| ATOM | 1873 | NE | ARG | 1126 | 1.605 | 22.899 | 18.230 | 1.00 | 52.07 |
| ATOM | 1874 | CZ | ARG | 1126 | 1.268 | 23.819 | 19.166 | 1.00 | 55.60 |
| ATOM | 1875 | NH1 | ARG | 1126 | 1.298 | 23.537 | 20.491 | 1.00 | 56.12 |
| ATOM | 1876 | NH2 | ARG | 1126 | 0.897 | 25.051 | 18.803 | 1.00 | 56.08 |
| ATOM | 1877 | C | ARG | 1126 | 4.011 | 23.950 | 14.372 | 1.00 | 33.43 |
| ATOM | 1878 | O | ARG | 1126 | 4.807 | 23.907 | 15.310 | 1.00 | 35.75 |
| ATOM | 1879 | N | ALA | 1127 | 3.647 | 25.144 | 13.914 | 1.00 | 30.99 |
| ATOM | 1880 | CA | ALA | 1127 | 4.219 | 26.377 | 14.527 | 1.00 | 27.32 |
| ATOM | 1881 | CB | ALA | 1127 | 3.951 | 27.619 | 13.636 | 1.00 | 26.53 |
| ATOM | 1882 | C | ALA | 1127 | 3.628 | 26.646 | 15.930 | 1.00 | 26.08 |
| ATOM | 1883 | O | ALA | 1127 | 2.459 | 26.318 | 16.205 | 1.00 | 28.40 |
| ATOM | 1884 | N | PRO | 1128 | 4.415 | 27.135 | 16.907 | 1.00 | 20.84 |
| ATOM | 1885 | CD | PRO | 1128 | 5.860 | 27.223 | 16.901 | 1.00 | 18.41 |
| ATOM | 1886 | CA | PRO | 1128 | 3.810 | 27.388 | 18.206 | 1.00 | 17.74 |
| ATOM | 1887 | CB | PRO | 1128 | 4.997 | 27.513 | 19.107 | 1.00 | 18.57 |
| ATOM | 1888 | CG | PRO | 1128 | 6.120 | 27.943 | 18.190 | 1.00 | 19.66 |
| ATOM | 1889 | C | PRO | 1128 | 2.949 | 28.614 | 18.104 | 1.00 | 18.60 |
| ATOM | 1890 | O | PRO | 1128 | 3.110 | 29.422 | 17.208 | 1.00 | 16.70 |
| ATOM | 1891 | N | ASP | 1129 | 2.081 | 28.840 | 19.057 | 1.00 | 19.15 |
| ATOM | 1892 | CA | ASP | 1129 | 1.151 | 29.999 | 19.100 | 1.00 | 18.53 |
| ATOM | 1893 | CB | ASP | 1129 | 0.120 | 30.025 | 20.289 | 1.00 | 18.06 |
| ATOM | 1894 | CG | ASP | 1129 | −0.877 | 28.886 | 20.309 | 1.00 | 21.66 |
| ATOM | 1895 | OD1 | ASP | 1129 | −1.631 | 28.755 | 21.293 | 1.00 | 22.21 |
| ATOM | 1896 | OD2 | ASP | 1129 | −0.880 | 28.123 | 19.346 | 1.00 | 24.19 |
| ATOM | 1897 | C | ASP | 1129 | 1.694 | 31.375 | 19.226 | 1.00 | 19.44 |
| ATOM | 1898 | O | ASP | 1129 | 0.891 | 32.287 | 19.045 | 1.00 | 24.77 |
| ATOM | 1899 | N | TYR | 1130 | 2.939 | 31.600 | 19.653 | 1.00 | 18.48 |
| ATOM | 1900 | CA | TYR | 1130 | 3.418 | 32.996 | 19.857 | 1.00 | 15.24 |
| ATOM | 1901 | CB | TYR | 1130 | 3.990 | 33.140 | 21.314 | 1.00 | 12.08 |
| ATOM | 1902 | CG | TYR | 1130 | 2.952 | 32.666 | 22.221 | 1.00 | 11.37 |
| ATOM | 1903 | CD1 | TYR | 1130 | 2.957 | 31.365 | 22.729 | 1.00 | 11.13 |
| ATOM | 1904 | CE1 | TYR | 1130 | 1.920 | 30.946 | 23.566 | 1.00 | 9.76 |
| ATOM | 1905 | CD2 | TYR | 1130 | 1.926 | 33.551 | 22.535 | 1.00 | 8.41 |
| ATOM | 1906 | CE2 | TYR | 1130 | 0.911 | 33.121 | 23.369 | 1.00 | 10.40 |
| ATOM | 1907 | CZ | TYR | 1130 | 0.920 | 31.834 | 23.876 | 1.00 | 7.94 |
| ATOM | 1908 | OH | TYR | 1130 | −0.083 | 31.477 | 24.744 | 1.00 | 16.20 |
| ATOM | 1909 | C | TYR | 1130 | 4.425 | 33.478 | 18.896 | 1.00 | 15.77 |
| ATOM | 1910 | O | TYR | 1130 | 4.800 | 34.648 | 18.850 | 1.00 | 18.09 |
| ATOM | 1911 | N | THR | 1131 | 4.887 | 32.500 | 18.179 | 1.00 | 16.94 |
| ATOM | 1912 | CA | THR | 1131 | 5.941 | 32.648 | 17.202 | 1.00 | 18.99 |
| ATOM | 1913 | CB | THR | 1131 | 6.253 | 31.291 | 16.603 | 1.00 | 16.51 |
| ATOM | 1914 | OG1 | THR | 1131 | 7.560 | 31.420 | 16.151 | 1.00 | 16.94 |
| ATOM | 1915 | CG2 | THR | 1131 | 5.409 | 30.863 | 15.361 | 1.00 | 19.81 |
| ATOM | 1916 | C | THR | 1131 | 5.781 | 33.573 | 16.061 | 1.00 | 20.79 |
| ATOM | 1917 | O | THR | 1131 | 4.669 | 33.993 | 15.726 | 1.00 | 24.01 |
| ATOM | 1918 | N | THR | 1132 | 6.914 | 33.872 | 15.442 | 1.00 | 21.24 |
| ATOM | 1919 | CA | THR | 1132 | 6.896 | 34.695 | 14.251 | 1.00 | 25.34 |
| ATOM | 1920 | CB | THR | 1132 | 7.980 | 35.734 | 14.326 | 1.00 | 26.52 |
| ATOM | 1921 | OG1 | THR | 1132 | 9.238 | 35.089 | 14.400 | 1.00 | 26.14 |
| ATOM | 1922 | CG2 | THR | 1132 | 7.709 | 36.687 | 15.486 | 1.00 | 28.70 |
| ATOM | 1923 | C | THR | 1132 | 7.097 | 33.826 | 12.993 | 1.00 | 29.71 |
| ATOM | 1924 | O | THR | 1132 | 7.497 | 32.651 | 13.033 | 1.00 | 32.05 |
| ATOM | 1925 | N | PRO | 1133 | 6.801 | 34.330 | 11.784 | 1.00 | 33.58 |
| ATOM | 1926 | CD | PRO | 1133 | 6.261 | 35.675 | 11.398 | 1.00 | 31.50 |
| ATOM | 1927 | CA | PRO | 1133 | 7.047 | 33.475 | 10.603 | 1.00 | 31.44 |
| ATOM | 1928 | CB | PRO | 1133 | 6.611 | 34.397 | 9.431 | 1.00 | 32.59 |
| ATOM | 1929 | CG | PRO | 1133 | 5.640 | 35.409 | 10.046 | 1.00 | 30.34 |
| ATOM | 1930 | C | PRO | 1133 | 8.523 | 33.061 | 10.572 | 1.00 | 30.59 |
| ATOM | 1931 | O | PRO | 1133 | 8.897 | 31.910 | 10.544 | 1.00 | 30.02 |
| ATOM | 1932 | N | GLU | 1134 | 9.417 | 34.037 | 10.621 | 1.00 | 30.18 |
| ATOM | 1933 | CA | GLU | 1134 | 10.850 | 33.752 | 10.545 | 1.00 | 31.71 |
| ATOM | 1934 | CB | GLU | 1134 | 11.578 | 35.125 | 10.322 | 1.00 | 30.12 |
| ATOM | 1935 | CG | GLU | 1134 | 11.280 | 36.416 | 11.087 | 1.00 | 30.33 |
| ATOM | 1936 | CD | GLU | 1134 | 10.099 | 37.199 | 10.559 | 1.00 | 29.05 |
| ATOM | 1937 | OE1 | GLU | 1134 | 9.010 | 37.030 | 11.083 | 1.00 | 30.75 |
| ATOM | 1938 | OE2 | GLU | 1134 | 10.251 | 37.988 | 9.631 | 1.00 | 30.95 |
| ATOM | 1939 | C | GLU | 1134 | 11.430 | 32.940 | 11.725 | 1.00 | 35.77 |
| ATOM | 1940 | O | GLU | 1134 | 12.386 | 32.133 | 11.577 | 1.00 | 38.27 |
| ATOM | 1941 | N | MET | 1135 | 10.862 | 33.165 | 12.943 | 1.00 | 34.45 |
| ATOM | 1942 | CA | MET | 1135 | 11.312 | 32.380 | 14.100 | 1.00 | 31.53 |
| ATOM | 1943 | CB | MET | 1135 | 10.595 | 32.816 | 15.384 | 1.00 | 29.77 |
| ATOM | 1944 | CG | MET | 1135 | 11.405 | 32.529 | 16.631 | 1.00 | 25.99 |
| ATOM | 1945 | SD | MET | 1135 | 12.845 | 33.570 | 16.615 | 1.00 | 20.76 |
| ATOM | 1946 | CE | MET | 1135 | 14.038 | 32.260 | 16.714 | 1.00 | 16.34 |
| ATOM | 1947 | C | MET | 1135 | 10.946 | 30.922 | 13.774 | 1.00 | 30.41 |
| ATOM | 1948 | O | MET | 1135 | 11.790 | 30.035 | 13.873 | 1.00 | 33.85 |
| ATOM | 1949 | N | TYR | 1136 | 9.725 | 30.613 | 13.337 | 1.00 | 27.63 |
| ATOM | 1950 | CA | TYR | 1136 | 9.406 | 29.211 | 12.994 | 1.00 | 27.32 |
| ATOM | 1951 | CB | TYR | 1136 | 7.897 | 29.098 | 12.665 | 1.00 | 29.58 |
| ATOM | 1952 | CG | TYR | 1136 | 7.520 | 27.693 | 12.389 | 1.00 | 30.11 |
| ATOM | 1953 | CD1 | TYR | 1136 | 7.797 | 26.583 | 13.227 | 1.00 | 26.27 |
| ATOM | 1954 | CE1 | TYR | 1136 | 7.369 | 25.297 | 12.825 | 1.00 | 25.39 |
| ATOM | 1955 | CD2 | TYR | 1136 | 6.827 | 27.497 | 11.190 | 1.00 | 31.39 |
| ATOM | 1956 | CE2 | TYR | 1136 | 6.411 | 26.217 | 10.807 | 1.00 | 26.57 |
| ATOM | 1957 | CZ | TYR | 1136 | 6.682 | 25.140 | 11.609 | 1.00 | 24.14 |
| ATOM | 1958 | OH | TYR | 1136 | 6.293 | 23.942 | 11.096 | 1.00 | 24.08 |
| ATOM | 1959 | C | TYR | 1136 | 10.271 | 28.653 | 11.843 | 1.00 | 26.50 |
| ATOM | 1960 | O | TYR | 1136 | 10.583 | 27.445 | 11.796 | 1.00 | 28.76 |
| ATOM | 1961 | N | GLN | 1137 | 10.662 | 29.501 | 10.889 | 1.00 | 24.07 |
| ATOM | 1962 | CA | GLN | 1137 | 11.528 | 29.025 | 9.824 | 1.00 | 21.80 |
| ATOM | 1963 | CB | GLN | 1137 | 11.717 | 30.110 | 8.725 | 1.00 | 28.73 |
| ATOM | 1964 | CG | GLN | 1137 | 12.676 | 29.642 | 7.559 | 1.00 | 29.75 |
| ATOM | 1965 | CD | GLN | 1137 | 12.168 | 28.310 | 7.061 | 1.00 | 31.06 |
| ATOM | 1966 | OE1 | GLN | 1137 | 10.979 | 28.188 | 6.777 | 1.00 | 34.41 |
| ATOM | 1967 | NE2 | GLN | 1137 | 12.968 | 27.236 | 6.976 | 1.00 | 28.18 |
| ATOM | 1968 | C | GLN | 1137 | 12.852 | 28.661 | 10.442 | 1.00 | 20.21 |
| ATOM | 1969 | O | GLN | 1137 | 13.540 | 27.736 | 10.010 | 1.00 | 16.42 |
| ATOM | 1970 | N | THR | 1138 | 13.300 | 29.415 | 11.452 | 1.00 | 19.75 |
| ATOM | 1971 | CA | THR | 1138 | 14.572 | 29.043 | 12.134 | 1.00 | 18.98 |
| ATOM | 1972 | CB | THR | 1138 | 14.950 | 30.125 | 13.160 | 1.00 | 16.69 |
| ATOM | 1973 | OG1 | THR | 1138 | 15.003 | 31.352 | 12.492 | 1.00 | 15.14 |
| ATOM | 1974 | CG2 | THR | 1138 | 16.352 | 29.949 | 13.742 | 1.00 | 17.82 |
| ATOM | 1975 | C | THR | 1138 | 14.412 | 27.654 | 12.843 | 1.00 | 20.29 |
| ATOM | 1976 | O | THR | 1138 | 15.276 | 26.785 | 12.718 | 1.00 | 19.06 |
| ATOM | 1977 | N | MET | 1139 | 13.312 | 27.364 | 13.559 | 1.00 | 19.20 |
| ATOM | 1978 | CA | MET | 1139 | 13.140 | 26.073 | 14.213 | 1.00 | 19.36 |
| ATOM | 1979 | CB | MET | 1139 | 11.728 | 26.079 | 14.805 | 1.00 | 14.66 |
| ATOM | 1980 | CG | MET | 1139 | 11.650 | 27.007 | 15.980 | 1.00 | 10.11 |
| ATOM | 1981 | SD | MET | 1139 | 9.902 | 27.182 | 16.388 | 1.00 | 9.11 |
| ATOM | 1982 | CE | MET | 1139 | 9.681 | 28.895 | 16.877 | 1.00 | 10.25 |
| ATOM | 1983 | C | MET | 1139 | 13.353 | 24.999 | 13.120 | 1.00 | 23.19 |
| ATOM | 1984 | O | MET | 1139 | 14.265 | 24.147 | 13.215 | 1.00 | 24.78 |
| ATOM | 1985 | N | LEU | 1140 | 12.590 | 25.119 | 11.991 | 1.00 | 25.23 |
| ATOM | 1986 | CA | LEU | 1140 | 12.680 | 24.213 | 10.775 | 1.00 | 23.17 |
| ATOM | 1987 | CB | LEU | 1140 | 11.919 | 24.787 | 9.621 | 1.00 | 20.44 |
| ATOM | 1988 | CG | LEU | 1140 | 10.426 | 24.839 | 9.857 | 1.00 | 20.18 |
| ATOM | 1989 | CD1 | LEU | 1140 | 9.800 | 25.529 | 8.605 | 1.00 | 20.32 |
| ATOM | 1990 | CD2 | LEU | 1140 | 9.847 | 23.440 | 10.032 | 1.00 | 19.94 |
| ATOM | 1991 | C | LEU | 1140 | 14.089 | 23.974 | 10.261 | 1.00 | 22.12 |
| ATOM | 1992 | O | LEU | 1140 | 14.513 | 22.880 | 9.888 | 1.00 | 23.91 |
| ATOM | 1993 | N | ASP | 1141 | 14.873 | 25.035 | 10.155 | 1.00 | 21.46 |
| ATOM | 1994 | CA | ASP | 1141 | 16.269 | 24.896 | 9.707 | 1.00 | 21.14 |
| ATOM | 1995 | CB | ASP | 1141 | 17.149 | 26.146 | 9.725 | 1.00 | 19.85 |
| ATOM | 1996 | CG | ASP | 1141 | 16.766 | 27.391 | 9.012 | 1.00 | 21.57 |
| ATOM | 1997 | OD1 | ASP | 1141 | 15.638 | 27.581 | 8.613 | 1.00 | 24.58 |
| ATOM | 1998 | OD2 | ASP | 1141 | 17.641 | 28.231 | 8.906 | 1.00 | 24.23 |
| ATOM | 1999 | C | ASP | 1141 | 17.085 | 24.043 | 10.667 | 1.00 | 23.75 |
| ATOM | 2000 | O | ASP | 1141 | 18.013 | 23.356 | 10.233 | 1.00 | 25.64 |
| ATOM | 2001 | N | CYS | 1142 | 16.867 | 24.299 | 12.001 | 1.00 | 23.77 |
| ATOM | 2002 | CA | CYS | 1142 | 17.580 | 23.686 | 13.182 | 1.00 | 21.74 |
| ATOM | 2003 | CB | CYS | 1142 | 17.096 | 24.320 | 14.535 | 1.00 | 20.04 |
| ATOM | 2004 | SG | CYS | 1142 | 17.680 | 26.043 | 14.896 | 1.00 | 17.71 |
| ATOM | 2005 | C | CYS | 1142 | 17.405 | 22.234 | 13.229 | 1.00 | 22.12 |
| ATOM | 2006 | O | CYS | 1142 | 18.239 | 21.416 | 13.592 | 1.00 | 22.29 |
| ATOM | 2007 | N | TRP | 1143 | 16.211 | 21.985 | 12.833 | 1.00 | 22.98 |
| ATOM | 2008 | CA | TRP | 1143 | 15.698 | 20.654 | 12.659 | 1.00 | 26.78 |
| ATOM | 2009 | CB | TRP | 1143 | 14.180 | 20.766 | 12.883 | 1.00 | 26.48 |
| ATOM | 2010 | CG | TRP | 1143 | 13.756 | 21.260 | 14.208 | 1.00 | 22.40 |
| ATOM | 2011 | CD2 | TRP | 1143 | 12.462 | 21.752 | 14.490 | 1.00 | 22.01 |
| ATOM | 2012 | CE2 | TRP | 1143 | 12.371 | 21.846 | 15.903 | 1.00 | 21.77 |
| ATOM | 2013 | CE3 | TRP | 1143 | 11.377 | 22.124 | 13.678 | 1.00 | 18.40 |
| ATOM | 2014 | CD1 | TRP | 1143 | 14.401 | 21.078 | 15.433 | 1.00 | 21.05 |
| ATOM | 2015 | NE1 | TRP | 1143 | 13.561 | 21.428 | 16.447 | 1.00 | 25.36 |
| ATOM | 2016 | CZ2 | TRP | 1143 | 11.193 | 22.313 | 16.467 | 1.00 | 15.80 |
| ATOM | 2017 | CZ3 | TRP | 1143 | 10.196 | 22.593 | 14.248 | 1.00 | 17.09 |
| ATOM | 2018 | CH2 | TRP | 1143 | 10.110 | 22.685 | 15.631 | 1.00 | 18.53 |
| ATOM | 2019 | C | TRP | 1143 | 16.043 | 20.051 | 11.247 | 1.00 | 28.20 |
| ATOM | 2020 | O | TRP | 1143 | 15.517 | 19.024 | 10.838 | 1.00 | 32.11 |
| ATOM | 2021 | N | HIS | 1144 | 16.860 | 20.647 | 10.417 | 1.00 | 28.22 |
| ATOM | 2022 | CA | HIS | 1144 | 17.203 | 20.116 | 9.096 | 1.00 | 30.82 |
| ATOM | 2023 | CB | HIS | 1144 | 18.347 | 21.003 | 8.495 | 1.00 | 32.30 |
| ATOM | 2024 | CG | HIS | 1144 | 18.727 | 20.542 | 7.186 | 1.00 | 33.69 |
| ATOM | 2025 | CD2 | HIS | 1144 | 19.625 | 19.538 | 6.889 | 1.00 | 35.14 |
| ATOM | 2026 | ND1 | HIS | 1144 | 18.095 | 20.892 | 6.053 | 1.00 | 35.37 |
| ATOM | 2027 | CE1 | HIS | 1144 | 18.585 | 20.102 | 5.055 | 1.00 | 34.51 |
| ATOM | 2028 | NE2 | HIS | 1144 | 19.522 | 19.267 | 5.564 | 1.00 | 35.38 |
| ATOM | 2029 | C | HIS | 1144 | 17.663 | 18.662 | 9.132 | 1.00 | 34.49 |
| ATOM | 2030 | O | HIS | 1144 | 18.711 | 18.404 | 9.693 | 1.00 | 38.32 |
| ATOM | 2031 | N | GLY | 1145 | 17.042 | 17.683 | 8.442 | 1.00 | 37.28 |
| ATOM | 2032 | CA | GLY | 1145 | 17.485 | 16.264 | 8.444 | 1.00 | 34.83 |
| ATOM | 2033 | C | GLY | 1145 | 19.008 | 16.085 | 8.490 | 1.00 | 37.11 |
| ATOM | 2034 | O | GLY | 1145 | 19.517 | 15.304 | 9.291 | 1.00 | 38.67 |
| ATOM | 2035 | N | GLU | 1146 | 19.791 | 16.769 | 7.634 | 1.00 | 36.69 |
| ATOM | 2036 | CA | GLU | 1146 | 21.291 | 16.640 | 7.637 | 1.00 | 38.08 |
| ATOM | 2037 | CB | GLU | 1146 | 21.915 | 16.850 | 6.196 | 1.00 | 40.29 |
| ATOM | 2038 | CG | GLU | 1146 | 21.765 | 15.651 | 5.269 | 1.00 | 49.47 |
| ATOM | 2039 | CD | GLU | 1146 | 20.376 | 15.491 | 4.595 | 1.00 | 56.82 |
| ATOM | 2040 | OE1 | GLU | 1146 | 19.319 | 15.427 | 5.252 | 1.00 | 60.62 |
| ATOM | 2041 | OE2 | GLU | 1146 | 20.372 | 15.389 | 3.359 | 1.00 | 62.18 |
| ATOM | 2042 | C | GLU | 1146 | 22.076 | 17.576 | 8.585 | 1.00 | 37.28 |
| ATOM | 2043 | O | GLU | 1146 | 22.125 | 18.783 | 8.327 | 1.00 | 36.45 |
| ATOM | 2044 | N | PRO | 1147 | 22.777 | 17.063 | 9.622 | 1.00 | 35.77 |
| ATOM | 2045 | CD | PRO | 1147 | 22.677 | 15.741 | 10.240 | 1.00 | 33.95 |
| ATOM | 2046 | CA | PRO | 1147 | 23.542 | 17.872 | 10.551 | 1.00 | 36.83 |
| ATOM | 2047 | CB | PRO | 1147 | 24.490 | 16.898 | 11.289 | 1.00 | 35.01 |
| ATOM | 2048 | CG | PRO | 1147 | 24.103 | 15.552 | 10.757 | 1.00 | 31.35 |
| ATOM | 2049 | C | PRO | 1147 | 24.277 | 19.031 | 9.944 | 1.00 | 38.83 |
| ATOM | 2050 | O | PRO | 1147 | 24.013 | 20.177 | 10.318 | 1.00 | 41.46 |
| ATOM | 2051 | N | SER | 1148 | 25.147 | 18.815 | 8.974 | 1.00 | 40.29 |
| ATOM | 2052 | CA | SER | 1148 | 25.841 | 19.994 | 8.446 | 1.00 | 40.87 |
| ATOM | 2053 | CB | SER | 1148 | 27.003 | 19.604 | 7.563 | 1.00 | 45.40 |
| ATOM | 2054 | OG | SER | 1148 | 28.175 | 20.127 | 8.216 | 1.00 | 52.17 |
| ATOM | 2055 | C | SER | 1148 | 25.003 | 21.011 | 7.697 | 1.00 | 39.76 |
| ATOM | 2056 | O | SER | 1148 | 25.526 | 22.081 | 7.437 | 1.00 | 39.54 |
| ATOM | 2057 | N | GLN | 1149 | 23.784 | 20.735 | 7.209 | 1.00 | 38.94 |
| ATOM | 2058 | CA | GLN | 1149 | 23.014 | 21.849 | 6.603 | 1.00 | 38.99 |
| ATOM | 2059 | CB | GLN | 1149 | 22.075 | 21.350 | 5.471 | 1.00 | 44.36 |
| ATOM | 2060 | CG | GLN | 1149 | 22.647 | 21.342 | 4.000 | 1.00 | 49.23 |
| ATOM | 2061 | CD | GLN | 1149 | 22.080 | 20.064 | 3.318 | 1.00 | 54.91 |
| ATOM | 2062 | OE1 | GLN | 1149 | 22.609 | 18.951 | 3.403 | 1.00 | 56.67 |
| ATOM | 2063 | NE2 | GLN | 1149 | 20.915 | 20.085 | 2.704 | 1.00 | 55.36 |
| ATOM | 2064 | C | GLN | 1149 | 22.171 | 22.600 | 7.692 | 1.00 | 36.18 |
| ATOM | 2065 | O | GLN | 1149 | 21.268 | 23.405 | 7.408 | 1.00 | 38.27 |
| ATOM | 2066 | N | ARG | 1150 | 22.310 | 22.231 | 8.986 | 1.00 | 31.34 |
| ATOM | 2067 | CA | ARG | 1150 | 21.628 | 22.948 | 10.097 | 1.00 | 22.73 |
| ATOM | 2068 | CB | ARG | 1150 | 21.703 | 22.203 | 11.331 | 1.00 | 19.96 |
| ATOM | 2069 | CG | ARG | 1150 | 20.616 | 21.235 | 11.301 | 1.00 | 17.26 |
| ATOM | 2070 | CD | ARG | 1150 | 20.801 | 20.399 | 12.535 | 1.00 | 16.38 |
| ATOM | 2071 | NE | ARG | 1150 | 20.150 | 19.160 | 12.180 | 1.00 | 16.05 |
| ATOM | 2072 | CZ | ARG | 1150 | 20.383 | 17.993 | 12.762 | 1.00 | 16.49 |
| ATOM | 2073 | NH1 | ARG | 1150 | 21.248 | 17.872 | 13.759 | 1.00 | 11.29 |
| ATOM | 2074 | NH2 | ARG | 1150 | 19.758 | 16.919 | 12.272 | 1.00 | 17.12 |
| ATOM | 2075 | C | ARG | 1150 | 22.456 | 24.184 | 10.318 | 1.00 | 20.39 |
| ATOM | 2076 | O | ARG | 1150 | 23.681 | 24.118 | 10.091 | 1.00 | 24.40 |
| ATOM | 2077 | N | PRO | 1151 | 21.933 | 25.330 | 10.750 | 1.00 | 16.34 |
| ATOM | 2078 | CD | PRO | 1151 | 20.564 | 25.614 | 11.187 | 1.00 | 16.79 |
| ATOM | 2079 | CA | PRO | 1151 | 22.826 | 26.481 | 10.963 | 1.00 | 14.97 |
| ATOM | 2080 | CB | PRO | 1151 | 21.852 | 27.632 | 11.173 | 1.00 | 11.25 |
| ATOM | 2081 | CG | PRO | 1151 | 20.620 | 27.025 | 11.804 | 1.00 | 12.29 |
| ATOM | 2082 | C | PRO | 1151 | 23.827 | 26.275 | 12.121 | 1.00 | 14.50 |
| ATOM | 2083 | O | PRO | 1151 | 23.783 | 25.282 | 12.865 | 1.00 | 14.95 |
| ATOM | 2084 | N | THR | 1152 | 24.834 | 27.118 | 12.225 | 1.00 | 11.46 |
| ATOM | 2085 | CA | THR | 1152 | 25.712 | 26.924 | 13.373 | 1.00 | 13.41 |
| ATOM | 2086 | CB | THR | 1152 | 27.160 | 27.324 | 13.101 | 1.00 | 10.17 |
| ATOM | 2087 | OG1 | THR | 1152 | 27.066 | 28.629 | 12.651 | 1.00 | 17.68 |
| ATOM | 2088 | CG2 | THR | 1152 | 27.908 | 26.539 | 12.077 | 1.00 | 9.09 |
| ATOM | 2089 | C | THR | 1152 | 25.233 | 27.825 | 14.528 | 1.00 | 15.56 |
| ATOM | 2090 | O | THR | 1152 | 24.272 | 28.614 | 14.401 | 1.00 | 20.97 |
| ATOM | 2091 | N | PHE | 1153 | 25.814 | 27.751 | 15.726 | 1.00 | 14.35 |
| ATOM | 2092 | CA | PHE | 1153 | 25.378 | 28.686 | 16.762 | 1.00 | 14.06 |
| ATOM | 2093 | CB | PHE | 1153 | 25.931 | 28.239 | 18.191 | 1.00 | 18.95 |
| ATOM | 2094 | CG | PHE | 1153 | 25.039 | 27.192 | 18.794 | 1.00 | 18.55 |
| ATOM | 2095 | CD1 | PHE | 1153 | 23.744 | 27.535 | 19.253 | 1.00 | 14.58 |
| ATOM | 2096 | CD2 | PHE | 1153 | 25.465 | 25.842 | 18.869 | 1.00 | 15.25 |
| ATOM | 2097 | CE1 | PHE | 1153 | 22.875 | 26.554 | 19.779 | 1.00 | 11.41 |
| ATOM | 2098 | CE2 | PHE | 1153 | 24.609 | 24.846 | 19.377 | 1.00 | 12.08 |
| ATOM | 2099 | CZ | PHE | 1153 | 23.323 | 25.210 | 19.827 | 1.00 | 16.51 |
| ATOM | 2100 | C | PHE | 1153 | 25.889 | 30.072 | 16.359 | 1.00 | 15.24 |
| ATOM | 2101 | O | PHE | 1153 | 25.239 | 31.060 | 16.665 | 1.00 | 12.33 |
| ATOM | 2102 | N | SER | 1154 | 27.055 | 30.269 | 15.701 | 1.00 | 18.36 |
| ATOM | 2103 | CA | SER | 1154 | 27.479 | 31.667 | 15.305 | 1.00 | 21.07 |
| ATOM | 2104 | CB | SER | 1154 | 28.947 | 31.643 | 14.859 | 1.00 | 20.62 |
| ATOM | 2105 | OG | SER | 1154 | 29.772 | 31.146 | 15.936 | 1.00 | 17.53 |
| ATOM | 2106 | C | SER | 1154 | 26.584 | 32.324 | 14.228 | 1.00 | 20.86 |
| ATOM | 2107 | O | SER | 1154 | 26.354 | 33.522 | 14.112 | 1.00 | 24.10 |
| ATOM | 2108 | N | GLU | 1155 | 26.039 | 31.503 | 13.384 | 1.00 | 20.15 |
| ATOM | 2109 | CA | GLU | 1155 | 25.067 | 31.953 | 12.378 | 1.00 | 18.73 |
| ATOM | 2110 | CB | GLU | 1155 | 24.475 | 30.881 | 11.465 | 1.00 | 20.78 |
| ATOM | 2111 | CG | GLU | 1155 | 24.936 | 30.667 | 10.019 | 1.00 | 21.99 |
| ATOM | 2112 | CD | GLU | 1155 | 26.096 | 29.731 | 9.914 | 1.00 | 24.10 |
| ATOM | 2113 | OE1 | GLU | 1155 | 27.202 | 30.271 | 10.032 | 1.00 | 26.13 |
| ATOM | 2114 | OE2 | GLU | 1155 | 25.876 | 28.514 | 9.727 | 1.00 | 19.08 |
| ATOM | 2115 | C | GLU | 1155 | 23.883 | 32.284 | 13.195 | 1.00 | 15.11 |
| ATOM | 2116 | O | GLU | 1155 | 23.354 | 33.361 | 13.116 | 1.00 | 16.58 |
| ATOM | 2117 | N | LEU | 1156 | 23.412 | 31.350 | 13.993 | 1.00 | 13.09 |
| ATOM | 2118 | CA | LEU | 1156 | 22.214 | 31.641 | 14.803 | 1.00 | 15.40 |
| ATOM | 2119 | CB | LEU | 1156 | 21.853 | 30.434 | 15.738 | 1.00 | 14.56 |
| ATOM | 2120 | CG | LEU | 1156 | 21.182 | 29.203 | 15.074 | 1.00 | 12.64 |
| ATOM | 2121 | CD1 | LEU | 1156 | 21.345 | 28.025 | 16.028 | 1.00 | 9.29 |
| ATOM | 2122 | CD2 | LEU | 1156 | 19.690 | 29.473 | 14.765 | 1.00 | 8.32 |
| ATOM | 2123 | C | LEU | 1156 | 22.310 | 32.864 | 15.647 | 1.00 | 18.03 |
| ATOM | 2124 | O | LEU | 1156 | 21.293 | 33.512 | 15.880 | 1.00 | 21.26 |
| ATOM | 2125 | N | VAL | 1157 | 23.487 | 33.171 | 16.189 | 1.00 | 18.46 |
| ATOM | 2126 | CA | VAL | 1157 | 23.685 | 34.352 | 17.018 | 1.00 | 16.85 |
| ATOM | 2127 | CB | VAL | 1157 | 25.075 | 34.385 | 17.520 | 1.00 | 16.06 |
| ATOM | 2128 | CG1 | VAL | 1157 | 25.401 | 35.743 | 18.144 | 1.00 | 20.87 |
| ATOM | 2129 | CG2 | VAL | 1157 | 25.195 | 33.374 | 18.650 | 1.00 | 14.98 |
| ATOM | 2130 | C | VAL | 1157 | 23.449 | 35.576 | 16.203 | 1.00 | 21.66 |
| ATOM | 2131 | O | VAL | 1157 | 22.811 | 36.525 | 16.678 | 1.00 | 24.36 |
| ATOM | 2132 | N | GLU | 1158 | 24.043 | 35.618 | 14.991 | 1.00 | 25.20 |
| ATOM | 2133 | CA | GLU | 1158 | 23.877 | 36.748 | 13.978 | 1.00 | 24.35 |
| ATOM | 2134 | CB | GLU | 1158 | 24.666 | 36.401 | 12.676 | 1.00 | 27.45 |
| ATOM | 2135 | CG | GLU | 1158 | 25.739 | 37.333 | 12.078 | 1.00 | 33.37 |
| ATOM | 2136 | CD | GLU | 1158 | 25.917 | 37.054 | 10.554 | 1.00 | 37.65 |
| ATOM | 2137 | OE1 | GLU | 1158 | 26.149 | 35.917 | 10.107 | 1.00 | 37.59 |
| ATOM | 2138 | OE2 | GLU | 1158 | 25.838 | 38.031 | 9.805 | 1.00 | 41.18 |
| ATOM | 2139 | C | GLU | 1158 | 22.389 | 36.935 | 13.598 | 1.00 | 21.38 |
| ATOM | 2140 | O | GLU | 1158 | 21.715 | 37.947 | 13.777 | 1.00 | 20.86 |
| ATOM | 2141 | N | HIS | 1159 | 21.767 | 35.826 | 13.222 | 1.00 | 19.39 |
| ATOM | 2142 | CA | HIS | 1159 | 20.359 | 35.803 | 12.806 | 1.00 | 19.70 |
| ATOM | 2143 | CB | HIS | 1159 | 19.971 | 34.367 | 12.332 | 1.00 | 26.96 |
| ATOM | 2144 | CG | HIS | 1159 | 18.590 | 34.300 | 11.804 | 1.00 | 30.60 |
| ATOM | 2145 | CD2 | HIS | 1159 | 18.101 | 34.913 | 10.655 | 1.00 | 29.08 |
| ATOM | 2146 | ND1 | HIS | 1159 | 17.536 | 33.663 | 12.423 | 1.00 | 36.03 |
| ATOM | 2147 | CE1 | HIS | 1159 | 16.401 | 33.872 | 11.676 | 1.00 | 34.96 |
| ATOM | 2148 | NE2 | HIS | 1159 | 16.769 | 34.639 | 10.599 | 1.00 | 33.53 |
| ATOM | 2149 | C | HIS | 1159 | 19.498 | 36.223 | 13.901 | 1.00 | 17.99 |
| ATOM | 2150 | O | HIS | 1159 | 18.671 | 37.131 | 13.797 | 1.00 | 21.79 |
| ATOM | 2151 | N | LEU | 1160 | 19.646 | 35.577 | 14.992 | 1.00 | 17.03 |
| ATOM | 2152 | CA | LEU | 1160 | 18.778 | 36.019 | 16.067 | 1.00 | 18.85 |
| ATOM | 2153 | CB | LEU | 1160 | 19.053 | 35.100 | 17.283 | 1.00 | 19.27 |
| ATOM | 2154 | CG | LEU | 1160 | 18.270 | 33.788 | 17.137 | 1.00 | 15.79 |
| ATOM | 2155 | CD1 | LEU | 1160 | 18.853 | 32.807 | 18.102 | 1.00 | 17.35 |
| ATOM | 2156 | CD2 | LEU | 1160 | 16.774 | 34.016 | 17.327 | 1.00 | 13.16 |
| ATOM | 2157 | C | LEU | 1160 | 18.965 | 37.493 | 16.368 | 1.00 | 16.37 |
| ATOM | 2158 | O | LEU | 1160 | 17.989 | 38.169 | 16.668 | 1.00 | 19.14 |
| ATOM | 2159 | N | GLY | 1161 | 20.158 | 38.024 | 16.181 | 1.00 | 15.23 |
| ATOM | 2160 | CA | GLY | 1161 | 20.449 | 39.439 | 16.453 | 1.00 | 16.78 |
| ATOM | 2161 | C | GLY | 1161 | 19.769 | 40.317 | 15.448 | 1.00 | 18.30 |
| ATOM | 2162 | O | GLY | 1161 | 19.345 | 41.441 | 15.732 | 1.00 | 16.10 |
| ATOM | 2163 | N | ASN | 1162 | 19.741 | 39.827 | 14.218 | 1.00 | 20.13 |
| ATOM | 2164 | CA | ASN | 1162 | 19.049 | 40.581 | 13.156 | 1.00 | 21.79 |
| ATOM | 2165 | CB | ASN | 1162 | 19.076 | 39.869 | 11.799 | 1.00 | 18.49 |
| ATOM | 2166 | CG | ASN | 1162 | 20.460 | 40.037 | 11.217 | 1.00 | 22.76 |
| ATOM | 2167 | OD1 | ASN | 1162 | 21.196 | 40.979 | 11.494 | 1.00 | 26.08 |
| ATOM | 2168 | ND2 | ASN | 1162 | 20.941 | 39.166 | 10.344 | 1.00 | 25.98 |
| ATOM | 2169 | C | ASN | 1162 | 17.600 | 40.694 | 13.578 | 1.00 | 25.01 |
| ATOM | 2170 | O | ASN | 1162 | 17.039 | 41.784 | 13.675 | 1.00 | 27.11 |
| ATOM | 2171 | N | LEU | 1163 | 17.000 | 39.541 | 13.908 | 1.00 | 26.81 |
| ATOM | 2172 | CA | LEU | 1163 | 15.598 | 39.475 | 14.352 | 1.00 | 26.06 |
| ATOM | 2173 | CB | LEU | 1163 | 15.252 | 38.068 | 14.779 | 1.00 | 26.82 |
| ATOM | 2174 | CG | LEU | 1163 | 14.497 | 37.181 | 13.769 | 1.00 | 29.87 |
| ATOM | 2175 | CD1 | LEU | 1163 | 15.220 | 37.208 | 12.437 | 1.00 | 27.89 |
| ATOM | 2176 | CD2 | LEU | 1163 | 14.381 | 35.737 | 14.282 | 1.00 | 25.85 |
| ATOM | 2177 | C | LEU | 1163 | 15.396 | 40.364 | 15.545 | 1.00 | 27.45 |
| ATOM | 2178 | O | LEU | 1163 | 14.393 | 41.057 | 15.680 | 1.00 | 28.61 |
| ATOM | 2179 | N | LEU | 1164 | 16.325 | 40.378 | 16.499 | 1.00 | 29.01 |
| ATOM | 2180 | CA | LEU | 1164 | 16.137 | 41.225 | 17.698 | 1.00 | 30.58 |
| ATOM | 2181 | CB | LEU | 1164 | 17.220 | 40.841 | 18.643 | 1.00 | 26.27 |
| ATOM | 2182 | CG | LEU | 1164 | 16.980 | 41.333 | 20.085 | 1.00 | 28.78 |
| ATOM | 2183 | CD1 | LEU | 1164 | 15.524 | 41.221 | 20.659 | 1.00 | 29.20 |
| ATOM | 2184 | CD2 | LEU | 1164 | 17.951 | 40.464 | 20.892 | 1.00 | 28.57 |
| ATOM | 2185 | C | LEU | 1164 | 16.127 | 42.722 | 17.388 | 1.00 | 34.54 |
| ATOM | 2186 | O | LEU | 1164 | 15.365 | 43.545 | 17.915 | 1.00 | 34.40 |
| ATOM | 2187 | N | GLN | 1165 | 17.011 | 43.130 | 16.497 | 1.00 | 40.87 |
| ATOM | 2188 | CA | GLN | 1165 | 16.989 | 44.534 | 16.126 | 1.00 | 45.70 |
| ATOM | 2189 | CB | GLN | 1165 | 18.272 | 44.869 | 15.347 | 1.00 | 48.67 |
| ATOM | 2190 | CG | GLN | 1165 | 19.035 | 45.938 | 16.193 | 1.00 | 54.44 |
| ATOM | 2191 | CD | GLN | 1165 | 18.634 | 47.452 | 16.021 | 1.00 | 56.74 |
| ATOM | 2192 | OE1 | GLN | 1165 | 19.339 | 48.238 | 15.327 | 1.00 | 58.30 |
| ATOM | 2193 | NE2 | GLN | 1165 | 17.573 | 47.912 | 16.709 | 1.00 | 56.65 |
| ATOM | 2194 | C | GLN | 1165 | 15.719 | 44.807 | 15.309 | 1.00 | 47.29 |
| ATOM | 2195 | O | GLN | 1165 | 15.421 | 45.926 | 14.911 | 1.00 | 48.97 |
| ATOM | 2196 | N | ALA | 1166 | 14.892 | 43.815 | 15.015 | 1.00 | 48.93 |
| ATOM | 2197 | CA | ALA | 1166 | 13.662 | 44.137 | 14.282 | 1.00 | 50.37 |
| ATOM | 2198 | CB | ALA | 1166 | 13.528 | 43.140 | 13.143 | 1.00 | 52.25 |
| ATOM | 2199 | C | ALA | 1166 | 12.442 | 44.130 | 15.218 | 1.00 | 52.50 |
| ATOM | 2200 | O | ALA | 1166 | 11.476 | 43.374 | 15.175 | 1.00 | 52.45 |
| ATOM | 2201 | N | ASN | 1167 | 12.568 | 45.034 | 16.155 | 1.00 | 53.58 |
| ATOM | 2202 | CA | ASN | 1167 | 11.633 | 45.382 | 17.240 | 1.00 | 55.62 |
| ATOM | 2203 | CB | ASN | 1167 | 11.646 | 44.512 | 18.537 | 1.00 | 57.30 |
| ATOM | 2204 | CG | ASN | 1167 | 11.596 | 43.000 | 18.459 | 1.00 | 55.48 |
| ATOM | 2205 | OD1 | ASN | 1167 | 10.653 | 42.386 | 18.961 | 1.00 | 51.86 |
| ATOM | 2206 | ND2 | ASN | 1167 | 12.629 | 42.362 | 17.943 | 1.00 | 54.74 |
| ATOM | 2207 | C | ASN | 1167 | 12.304 | 46.684 | 17.691 | 1.00 | 57.91 |
| ATOM | 2208 | O | ASN | 1167 | 13.175 | 46.667 | 18.573 | 1.00 | 60.31 |
| ATOM | 2209 | N | ALA | 1168 | 12.027 | 47.730 | 16.895 | 1.00 | 60.59 |
| ATOM | 2210 | CA | ALA | 1168 | 12.471 | 49.169 | 16.950 | 1.00 | 61.01 |
| ATOM | 2211 | CB | ALA | 1168 | 13.017 | 49.509 | 18.407 | 1.00 | 60.83 |
| ATOM | 2212 | C | ALA | 1168 | 13.521 | 49.498 | 15.830 | 1.00 | 61.36 |
| ATOM | 2213 | O | ALA | 1168 | 14.451 | 50.309 | 15.985 | 1.00 | 60.69 |
| ATOM | 2214 | C1 | INH | 1 | 22.111 | 24.647 | 38.269 | 1.00 | 18.89 |
| ATOM | 2215 | C01 | INH | 1 | 19.219 | 26.322 | 40.216 | 1.00 | 14.62 |
| ATOM | 2216 | C02 | INH | 1 | 20.566 | 26.567 | 40.034 | 1.00 | 18.71 |
| ATOM | 2217 | C03 | INH | 1 | 18.387 | 27.383 | 40.549 | 1.00 | 8.83 |
| ATOM | 2218 | C04 | INH | 1 | 18.483 | 25.131 | 40.105 | 1.00 | 12.67 |
| ATOM | 2219 | N05 | INH | 1 | 21.002 | 27.838 | 40.213 | 1.00 | 15.06 |
| ATOM | 2220 | S06 | INH | 1 | 21.746 | 25.183 | 39.807 | 1.00 | 18.54 |
| ATOM | 2221 | N07 | INH | 1 | 17.174 | 26.923 | 40.700 | 1.00 | 8.70 |
| ATOM | 2222 | N08 | INH | 1 | 18.822 | 28.585 | 40.747 | 1.00 | 9.78 |
| ATOM | 2223 | N09 | INH | 1 | 17.205 | 25.527 | 40.413 | 1.00 | 13.78 |
| ATOM | 2224 | C10 | INH | 1 | 20.126 | 28.766 | 40.566 | 1.00 | 14.45 |
| ATOM | 2225 | C17 | INH | 1 | 25.260 | 23.030 | 39.747 | 1.00 | 9.14 |
| ATOM | 2226 | C18 | INH | 1 | 25.937 | 22.740 | 38.529 | 1.00 | 7.86 |
| ATOM | 2227 | C19 | INH | 1 | 25.239 | 23.038 | 37.318 | 1.00 | 10.46 |
| ATOM | 2228 | C20 | INH | 1 | 23.977 | 23.571 | 37.312 | 1.00 | 10.45 |
| ATOM | 2229 | C21 | INH | 1 | 23.344 | 23.875 | 38.470 | 1.00 | 12.31 |
| ATOM | 2230 | C22 | INH | 1 | 23.966 | 23.603 | 39.704 | 1.00 | 9.66 |
| ATOM | 2231 | C27 | INH | 1 | 25.689 | 22.928 | 35.954 | 1.00 | 9.35 |
| ATOM | 2232 | O28 | INH | 1 | 25.156 | 23.553 | 35.039 | 1.00 | 12.19 |
| ATOM | 2233 | N29 | INH | 1 | 26.420 | 21.863 | 35.640 | 1.00 | 14.38 |
| ATOM | 2234 | C31 | INH | 1 | 27.579 | 22.240 | 32.219 | 1.00 | 25.16 |
| ATOM | 2235 | C32 | INH | 1 | 27.855 | 20.969 | 31.780 | 1.00 | 22.38 |
| ATOM | 2236 | C33 | INH | 1 | 27.668 | 19.958 | 32.789 | 1.00 | 18.99 |
| ATOM | 2237 | C34 | INH | 1 | 27.207 | 20.260 | 34.102 | 1.00 | 13.62 |
| ATOM | 2238 | C35 | INH | 1 | 26.927 | 21.562 | 34.444 | 1.00 | 13.42 |
| ATOM | 2239 | C36 | INH | 1 | 27.126 | 22.522 | 33.494 | 1.00 | 19.32 |
| ATOM | 2240 | O39 | INH | 1 | 28.016 | 18.561 | 32.500 | 1.00 | 15.72 |
| ATOM | 2241 | C40 | INH | 1 | 28.533 | 17.687 | 33.512 | 1.00 | 18.86 |
| ATOM | 2242 | O44 | INH | 1 | 28.010 | 20.890 | 30.293 | 1.00 | 19.40 |
| ATOM | 2243 | C45 | INH | 1 | 27.893 | 19.628 | 29.662 | 1.00 | 17.17 |
| ATOM | 2244 | O49 | INH | 1 | 27.883 | 23.434 | 31.440 | 1.00 | 32.15 |
| ATOM | 2245 | C50 | INH | 1 | 29.093 | 24.166 | 32.026 | 1.00 | 35.35 |
| ATOM | 2246 | OH2 | WAT | 1 | 34.548 | 13.225 | 49.909 | 1.00 | 17.82 |
| ATOM | 2247 | OH2 | WAT | 2 | 37.315 | 12.852 | 45.457 | 1.00 | 49.58 |
| ATOM | 2248 | OH2 | WAT | 3 | 34.057 | 9.649 | 47.170 | 1.00 | 27.80 |
| ATOM | 2249 | OH2 | WAT | 4 | 36.618 | 32.171 | 43.575 | 1.00 | 34.60 |
| ATOM | 2250 | OH2 | WAT | 5 | 24.902 | 12.420 | 52.559 | 1.00 | 67.99 |
| ATOM | 2251 | OH2 | WAT | 6 | 17.961 | 20.786 | 52.109 | 1.00 | 14.76 |
| ATOM | 2252 | OH2 | WAT | 7 | 36.970 | 31.379 | 59.957 | 1.00 | 91.33 |
| ATOM | 2253 | OH2 | WAT | 8 | 24.995 | 39.600 | 55.161 | 1.00 | 81.74 |
| ATOM | 2254 | OH2 | WAT | 9 | 38.269 | 40.102 | 50.357 | 1.00 | 75.66 |
| ATOM | 2255 | OH2 | WAT | 10 | 30.093 | 13.334 | 47.716 | 1.00 | 11.54 |
| ATOM | 2256 | OH2 | WAT | 11 | 33.665 | 1.127 | 40.846 | 1.00 | 20.17 |
| ATOM | 2257 | OH2 | WAT | 12 | 27.986 | 1.215 | 41.907 | 1.00 | 33.44 |
| ATOM | 2258 | OH2 | WAT | 13 | 21.163 | 2.173 | 43.000 | 1.00 | 89.30 |
| ATOM | 2259 | OH2 | WAT | 14 | 41.561 | 27.639 | 37.269 | 1.00 | 35.73 |
| ATOM | 2260 | OH2 | WAT | 15 | 35.416 | 29.290 | 58.752 | 1.00 | 36.64 |
| ATOM | 2261 | OH2 | WAT | 16 | 26.476 | 33.936 | 54.738 | 1.00 | 18.37 |
| ATOM | 2262 | OH2 | WAT | 17 | 14.139 | 12.458 | 47.487 | 1.00 | 40.73 |
| ATOM | 2263 | OH2 | WAT | 18 | 22.649 | 35.507 | 39.101 | 1.00 | 22.24 |
| ATOM | 2264 | OH2 | WAT | 19 | 23.475 | 37.872 | 35.424 | 1.00 | 41.98 |
| ATOM | 2265 | OH2 | WAT | 20 | 22.800 | 40.683 | 35.743 | 1.00 | 61.82 |
| ATOM | 2266 | OH2 | WAT | 21 | 25.344 | 16.235 | 37.727 | 1.00 | 22.92 |
| ATOM | 2267 | OH2 | WAT | 22 | 26.923 | 14.538 | 35.426 | 1.00 | 13.72 |
| ATOM | 2268 | OH2 | WAT | 23 | 35.129 | 15.141 | 48.316 | 1.00 | 12.43 |
| ATOM | 2269 | OH2 | WAT | 24 | 9.675 | 31.738 | 38.547 | 1.00 | 38.07 |
| ATOM | 2270 | OH2 | WAT | 25 | 10.834 | 28.120 | 37.401 | 1.00 | 17.84 |
| ATOM | 2271 | OH2 | WAT | 26 | 1.245 | 22.482 | 27.779 | 1.00 | 23.20 |
| ATOM | 2272 | OH2 | WAT | 27 | 1.041 | 27.306 | 23.572 | 1.00 | 12.48 |
| ATOM | 2273 | OH2 | WAT | 28 | −0.208 | 23.913 | 25.123 | 1.00 | 48.47 |
| ATOM | 2274 | OH2 | WAT | 29 | 2.971 | 18.535 | 16.655 | 1.00 | 13.50 |
| ATOM | 2275 | OH2 | WAT | 30 | 9.948 | 19.475 | 14.797 | 1.00 | 17.51 |
| ATOM | 2276 | OH2 | WAT | 31 | 12.909 | 16.411 | 18.059 | 1.00 | 19.02 |
| ATOM | 2277 | OH2 | WAT | 32 | 21.326 | 19.212 | 23.334 | 1.00 | 20.45 |
| ATOM | 2278 | OH2 | WAT | 33 | 23.051 | 17.305 | 21.845 | 1.00 | 12.88 |
| ATOM | 2279 | OH2 | WAT | 34 | 19.124 | 16.054 | 24.066 | 1.00 | 5.97 |
| ATOM | 2280 | OH2 | WAT | 35 | 14.459 | 41.552 | 37.771 | 1.00 | 35.34 |
| ATOM | 2281 | OH2 | WAT | 36 | 9.755 | 21.262 | 31.584 | 1.00 | 43.82 |
| ATOM | 2282 | OH2 | WAT | 37 | 4.242 | 27.040 | 35.563 | 1.00 | 12.38 |
| ATOM | 2283 | OH2 | WAT | 38 | −1.550 | 37.527 | 28.220 | 1.00 | 29.59 |
| ATOM | 2284 | OH2 | WAT | 39 | 8.387 | 42.795 | 21.015 | 1.00 | 35.54 |
| ATOM | 2285 | OH2 | WAT | 40 | 32.166 | 21.338 | 20.282 | 1.00 | 17.46 |
| ATOM | 2286 | OH2 | WAT | 41 | 30.747 | 19.974 | 13.933 | 1.00 | 39.46 |
| ATOM | 2287 | OH2 | WAT | 42 | 28.825 | 22.320 | 10.764 | 1.00 | 32.63 |
| ATOM | 2288 | OH2 | WAT | 43 | 11.271 | 3.702 | 14.460 | 1.00 | 23.99 |
| ATOM | 2289 | OH2 | WAT | 44 | 12.866 | 20.860 | 8.939 | 1.00 | 29.40 |
| ATOM | 2290 | OH2 | WAT | 45 | 15.875 | 22.841 | 6.803 | 1.00 | 32.59 |
| ATOM | 2291 | OH2 | WAT | 46 | 7.195 | 30.535 | 8.890 | 1.00 | 23.27 |
| ATOM | 2292 | OH2 | WAT | 47 | 40.104 | 31.256 | 37.242 | 1.00 | 49.59 |
| ATOM | 2293 | OH2 | WAT | 48 | 10.447 | 26.480 | 39.494 | 1.00 | 54.13 |
| ATOM | 2294 | OH2 | WAT | 49 | 19.617 | 12.685 | 51.158 | 1.00 | 23.88 |
| ATOM | 2295 | OH2 | WAT | 50 | 29.496 | 14.639 | 35.050 | 1.00 | 30.50 |
| ATOM | 2296 | OH2 | WAT | 51 | 29.485 | 10.804 | 33.888 | 1.00 | 27.93 |
| ATOM | 2297 | OH2 | WAT | 52 | 35.485 | 6.629 | 36.473 | 1.00 | 24.71 |
| ATOM | 2298 | OH2 | WAT | 53 | 22.972 | 37.804 | 41.733 | 1.00 | 60.44 |
| ATOM | 2299 | OH2 | WAT | 54 | 27.912 | 15.258 | 47.568 | 1.00 | 16.89 |
| ATOM | 2300 | OH2 | WAT | 55 | 9.573 | 40.099 | 32.795 | 1.00 | 36.41 |
| ATOM | 2301 | OH2 | WAT | 56 | 31.829 | 29.420 | 18.915 | 1.00 | 27.06 |
| ATOM | 2302 | OH2 | WAT | 57 | 20.875 | 15.991 | 21.305 | 1.00 | 22.41 |
| ATOM | 2303 | OH2 | WAT | 58 | 33.908 | 21.274 | 45.151 | 1.00 | 46.19 |
| ATOM | 2304 | OH2 | WAT | 59 | 27.786 | 39.554 | 54.673 | 1.00 | 34.72 |
| ATOM | 2305 | OH2 | WAT | 60 | 12.415 | 19.544 | 45.380 | 1.00 | 33.19 |
| ATOM | 2306 | OH2 | WAT | 61 | 12.897 | 26.434 | 47.503 | 1.00 | 43.43 |
| ATOM | 2307 | OH2 | WAT | 62 | 7.303 | 29.961 | 38.627 | 1.00 | 32.94 |
| ATOM | 2308 | OH2 | WAT | 63 | 4.634 | 32.397 | 39.731 | 1.00 | 63.37 |
| ATOM | 2309 | OH2 | WAT | 64 | 30.766 | 41.023 | 43.053 | 1.00 | 38.25 |
| ATOM | 2310 | OH2 | WAT | 65 | 27.994 | 5.877 | 37.833 | 1.00 | 37.03 |
| ATOM | 2311 | OH2 | WAT | 66 | 19.894 | 11.274 | 40.490 | 1.00 | 28.73 |
| ATOM | 2312 | OH2 | WAT | 67 | 35.801 | 34.433 | 30.190 | 1.00 | 30.64 |
| ATOM | 2313 | OH2 | WAT | 68 | 33.161 | 34.563 | 27.654 | 1.00 | 15.69 |
| ATOM | 2314 | OH2 | WAT | 69 | 32.519 | 33.673 | 23.243 | 1.00 | 34.65 |
| ATOM | 2315 | OH2 | WAT | 70 | 31.291 | 27.869 | 33.852 | 1.00 | 42.56 |
| ATOM | 2316 | OH2 | WAT | 71 | 37.384 | 32.602 | 40.463 | 1.00 | 50.04 |
| ATOM | 2317 | OH2 | WAT | 72 | 33.223 | 35.278 | 40.861 | 1.00 | 38.82 |
| ATOM | 2318 | OH2 | WAT | 73 | 36.367 | 31.425 | 35.298 | 1.00 | 40.16 |
| ATOM | 2319 | OH2 | WAT | 74 | 37.149 | 32.714 | 32.019 | 1.00 | 34.69 |
| ATOM | 2320 | OH2 | WAT | 75 | 35.255 | 35.550 | 33.148 | 1.00 | 46.40 |
| ATOM | 2321 | OH2 | WAT | 76 | 36.715 | 31.014 | 28.864 | 1.00 | 23.59 |
| ATOM | 2322 | OH2 | WAT | 77 | 27.885 | 22.984 | 54.467 | 1.00 | 36.95 |
| ATOM | 2323 | OH2 | WAT | 78 | 29.082 | 16.425 | 56.776 | 1.00 | 74.08 |
| ATOM | 2324 | OH2 | WAT | 79 | 33.729 | 22.016 | 58.060 | 1.00 | 23.98 |
| ATOM | 2325 | OH2 | WAT | 80 | 34.288 | 19.816 | 56.188 | 1.00 | 51.00 |
| ATOM | 2326 | OH2 | WAT | 81 | 13.950 | 29.489 | 44.073 | 1.00 | 34.10 |
| ATOM | 2327 | OH2 | WAT | 82 | 22.270 | 37.019 | 46.062 | 1.00 | 47.86 |
| ATOM | 2328 | OH2 | WAT | 83 | 21.137 | 35.937 | 43.530 | 1.00 | 44.52 |
| ATOM | 2329 | OH2 | WAT | 84 | 22.700 | 38.577 | 50.997 | 1.00 | 50.63 |
| ATOM | 2330 | OH2 | WAT | 85 | 29.919 | 43.174 | 46.637 | 1.00 | 42.26 |
| ATOM | 2331 | OH2 | WAT | 86 | 30.803 | 7.962 | 36.842 | 1.00 | 43.83 |
| ATOM | 2332 | OH2 | WAT | 87 | 32.675 | 10.034 | 35.163 | 1.00 | 23.79 |
| ATOM | 2333 | OH2 | WAT | 88 | 31.328 | 15.413 | 33.442 | 1.00 | 28.01 |
| ATOM | 2334 | OH2 | WAT | 89 | 36.351 | 18.583 | 26.226 | 1.00 | 38.07 |
| ATOM | 2335 | OH2 | WAT | 90 | 34.675 | 15.932 | 28.920 | 1.00 | 29.96 |
| ATOM | 2336 | OH2 | WAT | 91 | 28.339 | 37.115 | 34.563 | 1.00 | 52.20 |
| ATOM | 2337 | OH2 | WAT | 92 | 13.909 | 38.705 | 34.715 | 1.00 | 33.12 |
| ATOM | 2338 | OH2 | WAT | 93 | 13.125 | 41.678 | 27.218 | 1.00 | 26.05 |
| ATOM | 2339 | OH2 | WAT | 94 | 17.016 | 42.631 | 25.980 | 1.00 | 25.19 |
| ATOM | 2340 | OH2 | WAT | 95 | 23.692 | 39.647 | 13.190 | 1.00 | 46.62 |
| ATOM | 2341 | OH2 | WAT | 96 | 22.410 | 35.350 | 9.379 | 1.00 | 15.22 |
| ATOM | 2342 | OH2 | WAT | 97 | 27.878 | 28.033 | 8.163 | 1.00 | 37.79 |
| ATOM | 2343 | OH2 | WAT | 98 | 29.630 | 26.067 | 16.072 | 1.00 | 44.86 |
| ATOM | 2344 | OH2 | WAT | 99 | 33.466 | 27.410 | 17.797 | 1.00 | 34.77 |
| ATOM | 2345 | OH2 | WAT | 100 | 8.200 | 23.543 | 28.215 | 1.00 | 44.46 |
| ATOM | 2346 | OH2 | WAT | 101 | 1.950 | 20.748 | 23.869 | 1.00 | 27.19 |
| ATOM | 2347 | OH2 | WAT | 102 | 2.290 | 26.581 | 21.091 | 1.00 | 8.02 |
| ATOM | 2348 | OH2 | WAT | 103 | 3.082 | 21.878 | 30.651 | 1.00 | 19.42 |
| ATOM | 2349 | OH2 | WAT | 104 | 5.051 | 25.075 | 39.296 | 1.00 | 50.74 |
| ATOM | 2350 | OH2 | WAT | 105 | 33.167 | 18.487 | 20.184 | 1.00 | 59.39 |
| ATOM | 2351 | OH2 | WAT | 106 | 9.441 | 12.543 | 26.459 | 1.00 | 29.76 |
| ATOM | 2352 | OH2 | WAT | 107 | 4.522 | 9.248 | 24.885 | 1.00 | 55.98 |
| ATOM | 2353 | OH2 | WAT | 108 | 1.759 | 4.482 | 16.285 | 1.00 | 63.56 |
| ATOM | 2354 | OH2 | WAT | 109 | 6.598 | 1.331 | 18.743 | 1.00 | 42.47 |
| ATOM | 2355 | OH2 | WAT | 110 | 11.663 | 1.991 | 17.719 | 1.00 | 58.51 |
| ATOM | 2356 | OH2 | WAT | 111 | 14.362 | 3.963 | 16.972 | 1.00 | 45.93 |
| ATOM | 2357 | OH2 | WAT | 112 | 0.105 | 18.678 | 17.030 | 1.00 | 48.45 |
| ATOM | 2358 | OH2 | WAT | 113 | 3.017 | 25.140 | 10.851 | 1.00 | 23.53 |
| ATOM | 2359 | OH2 | WAT | 114 | 5.477 | 27.691 | 8.000 | 1.00 | 47.79 |
| ATOM | 2360 | OH2 | WAT | 115 | 10.303 | 25.269 | 5.084 | 1.00 | 60.12 |
| ATOM | 2361 | OH2 | WAT | 116 | 5.786 | 22.265 | 9.161 | 1.00 | 51.44 |
| ATOM | 2362 | OH2 | WAT | 117 | 26.816 | 23.874 | 9.577 | 1.00 | 27.87 |
| ATOM | 2363 | OH2 | WAT | 118 | 31.291 | 18.845 | 10.672 | 1.00 | 40.15 |
| ATOM | 2364 | OH2 | WAT | 119 | 15.676 | 29.566 | 41.272 | 1.00 | 28.95 |
| ATOM | 2365 | OH2 | WAT | 120 | 14.466 | 26.135 | 40.842 | 1.00 | 35.20 |
| ATOM | 2366 | OH2 | WAT | 121 | 36.037 | 23.319 | 51.451 | 1.00 | 19.77 |
| ATOM | 2367 | OH2 | WAT | 122 | 39.581 | 26.288 | 51.952 | 1.00 | 23.51 |
| ATOM | 2368 | OH2 | WAT | 123 | 42.528 | 27.670 | 50.228 | 1.00 | 24.63 |
| ATOM | 2369 | OH2 | WAT | 124 | 44.546 | 23.926 | 49.052 | 1.00 | 57.61 |
| ATOM | 2370 | OH2 | WAT | 125 | 40.023 | 28.576 | 47.131 | 1.00 | 38.26 |
| ATOM | 2371 | OH2 | WAT | 126 | 30.006 | 22.980 | 58.432 | 1.00 | 43.07 |
| ATOM | 2372 | OH2 | WAT | 127 | 26.128 | 9.390 | 12.264 | 1.00 | 19.21 |
| ATOM | 2373 | OH2 | WAT | 128 | 27.172 | 6.564 | 12.815 | 1.00 | 35.46 |
| ATOM | 2374 | OH2 | WAT | 129 | 26.963 | 14.011 | 12.742 | 1.00 | 36.13 |
| ATOM | 2375 | OH2 | WAT | 130 | 28.303 | 18.295 | 14.404 | 1.00 | 28.96 |
| ATOM | 2376 | OH2 | WAT | 131 | 24.031 | 15.269 | 20.692 | 1.00 | 48.88 |
| ATOM | 2377 | OH2 | WAT | 132 | 29.264 | 9.703 | 19.566 | 1.00 | 53.12 |
| ATOM | 2378 | OH2 | WAT | 133 | 17.989 | 30.518 | 10.979 | 1.00 | 25.47 |
| ATOM | 2379 | OH2 | WAT | 134 | 11.990 | 39.778 | 11.046 | 1.00 | 38.98 |
| ATOM | 2380 | OH2 | WAT | 135 | 11.135 | 34.002 | 7.269 | 1.00 | 31.35 |
| ATOM | 2381 | OH2 | WAT | 136 | 3.675 | 32.379 | 11.688 | 1.00 | 42.88 |
| ATOM | 2382 | OH2 | WAT | 137 | 19.194 | 18.329 | 1.416 | 1.00 | 54.93 |
| ATOM | 2383 | OH2 | WAT | 138 | 29.343 | 36.491 | 19.061 | 1.00 | 57.53 |
| ATOM | 2384 | OH2 | WAT | 139 | 26.421 | 39.693 | 19.687 | 1.00 | 28.22 |
| ATOM | 2385 | OH2 | WAT | 140 | 23.298 | 41.897 | 17.546 | 1.00 | 27.34 |
| ATOM | 2386 | OH2 | WAT | 141 | 21.627 | 44.076 | 18.974 | 1.00 | 49.29 |
| ATOM | 2387 | OH2 | WAT | 142 | 19.030 | 44.490 | 22.407 | 1.00 | 30.22 |
| ATOM | 2388 | OH2 | WAT | 143 | 20.677 | 19.612 | 52.476 | 1.00 | 47.14 |
| ATOM | 2389 | OH2 | WAT | 144 | 19.488 | 16.859 | 51.913 | 1.00 | 48.24 |
| ATOM | 2390 | OH2 | WAT | 145 | 17.301 | 11.237 | 51.195 | 1.00 | 50.01 |
| ATOM | 2391 | OH2 | WAT | 146 | 13.220 | 10.587 | 40.098 | 1.00 | 43.73 |
| ATOM | 2392 | OH2 | WAT | 147 | 19.137 | 9.886 | 38.306 | 1.00 | 48.36 |
| ATOM | 2393 | OH2 | WAT | 148 | 8.558 | 15.164 | 38.060 | 1.00 | 39.18 |
| ATOM | 2394 | OH2 | WAT | 149 | 24.261 | 11.022 | 33.256 | 1.00 | 37.59 |
| ATOM | 2395 | OH2 | WAT | 150 | 25.989 | 17.330 | 35.066 | 1.00 | 44.49 |
| ATOM | 2396 | OH2 | WAT | 151 | 25.997 | 14.655 | 30.354 | 1.00 | 45.30 |
| ATOM | 2397 | OH2 | WAT | 152 | 21.169 | 15.266 | 31.358 | 1.00 | 31.99 |
| ATOM | 2398 | OH2 | WAT | 153 | 29.183 | 17.439 | 28.415 | 1.00 | 46.53 |
| ATOM | 2399 | OH2 | WAT | 154 | 36.461 | 15.340 | 27.070 | 1.00 | 40.62 |
| ATOM | 2400 | OH2 | WAT | 155 | 35.657 | 16.078 | 31.940 | 1.00 | 31.65 |
| ATOM | 2401 | OH2 | WAT | 156 | 24.883 | 42.087 | 33.016 | 1.00 | 48.49 |
| ATOM | 2402 | OH2 | WAT | 157 | 6.799 | 37.475 | 36.469 | 1.00 | 59.19 |
| ATOM | 2403 | OH2 | WAT | 158 | 10.345 | 41.204 | 37.883 | 1.00 | 49.50 |
| ATOM | 2404 | OH2 | WAT | 159 | 7.754 | 18.515 | 28.466 | 1.00 | 33.89 |
| ATOM | 2405 | OH2 | WAT | 160 | 12.133 | 12.815 | 25.496 | 1.00 | 20.58 |
| ATOM | 2406 | OH2 | WAT | 161 | 17.629 | 18.048 | 25.474 | 1.00 | 20.91 |
| ATOM | 2407 | OH2 | WAT | 162 | 32.150 | 24.842 | 17.417 | 1.00 | 45.27 |
| ATOM | 2408 | OH2 | WAT | 163 | 29.679 | 28.436 | 17.333 | 1.00 | 27.94 |
| ATOM | 2409 | OH2 | WAT | 164 | 29.482 | 31.430 | 8.188 | 1.00 | 37.43 |
| ATOM | 2410 | OH2 | WAT | 165 | 26.321 | 25.192 | 5.401 | 1.00 | 36.87 |
| ATOM | 2411 | OH2 | WAT | 166 | 20.427 | 31.093 | 10.497 | 1.00 | 28.10 |
| ATOM | 2412 | OH2 | WAT | 167 | 26.472 | 37.561 | 7.307 | 1.00 | 37.66 |
| ATOM | 2413 | OH2 | WAT | 168 | 19.311 | 43.549 | 36.760 | 1.00 | 47.90 |
| ATOM | 2414 | OH2 | WAT | 169 | 34.046 | 18.968 | 27.646 | 1.00 | 40.67 |
| ATOM | 2415 | OH2 | WAT | 170 | 32.372 | 16.850 | 27.752 | 1.00 | 40.96 |

TABLE 2
Atomic Coordinates for
VEGFR2KD: N-Methyl-2-[3-(2-pyridin-2-yl-vinyl)-1H-indazol-6-ylsulfanyl]-
benzamide (Compound 2)
Complex Crystalline Structure

| | | | | | | | | | |
|---|---|---|---|---|---|---|---|---|---|
| ATOM | 1 | C | HIS | 816 | 38.644 | 13.619 | 29.967 | 1.00 | 39.12 |
| ATOM | 2 | O | HIS | 816 | 39.770 | 14.108 | 30.067 | 1.00 | 38.61 |
| ATOM | 3 | CB | HIS | 816 | 39.606 | 12.376 | 27.867 | 1.00 | 40.54 |
| ATOM | 4 | CG | HIS | 816 | 39.466 | 11.129 | 27.057 | 1.00 | 43.56 |
| ATOM | 5 | ND1 | HIS | 816 | 38.313 | 10.803 | 26.354 | 1.00 | 46.11 |
| ATOM | 6 | CE1 | HIS | 816 | 38.509 | 9.543 | 25.792 | 1.00 | 44.26 |
| ATOM | 7 | CD2 | HIS | 816 | 40.376 | 10.060 | 26.912 | 1.00 | 43.38 |
| ATOM | 8 | NE2 | HIS | 816 | 39.772 | 9.107 | 26.142 | 1.00 | 42.62 |
| ATOM | 9 | HE2 | HIS | 816 | 40.155 | 8.253 | 25.844 | 1.00 | 0.00 |
| ATOM | 10 | HT1 | HIS | 816 | 37.097 | 13.765 | 27.602 | 1.00 | 0.00 |
| ATOM | 11 | HT2 | HIS | 816 | 36.297 | 12.523 | 28.477 | 1.00 | 0.00 |
| ATOM | 12 | N | HIS | 816 | 37.152 | 12.784 | 27.947 | 1.00 | 38.68 |
| ATOM | 13 | HT3 | HIS | 816 | 37.251 | 12.129 | 27.136 | 1.00 | 0.00 |
| ATOM | 14 | CA | HIS | 816 | 38.350 | 12.560 | 28.828 | 1.00 | 39.93 |
| ATOM | 15 | N | CYS | 817 | 37.654 | 13.991 | 30.856 | 1.00 | 38.18 |
| ATOM | 16 | H | CYS | 817 | 36.795 | 13.526 | 30.806 | 1.00 | 0.00 |
| ATOM | 17 | CA | CYS | 817 | 37.782 | 15.022 | 31.986 | 1.00 | 36.83 |
| ATOM | 18 | CB | CYS | 817 | 36.503 | 15.436 | 32.718 | 1.00 | 36.44 |
| ATOM | 19 | SG | CYS | 817 | 35.238 | 16.001 | 31.620 | 1.00 | 42.71 |
| ATOM | 20 | C | CYS | 817 | 38.617 | 14.641 | 33.186 | 1.00 | 35.03 |
| ATOM | 21 | O | CYS | 817 | 38.905 | 15.495 | 34.005 | 1.00 | 33.39 |
| ATOM | 22 | N | GLU | 818 | 38.819 | 13.326 | 33.384 | 1.00 | 32.41 |
| ATOM | 23 | H | GLU | 818 | 38.351 | 12.713 | 32.797 | 1.00 | 0.00 |
| ATOM | 24 | CA | GLU | 818 | 39.634 | 12.806 | 34.489 | 1.00 | 31.13 |
| ATOM | 25 | CB | GLU | 818 | 39.745 | 11.280 | 34.413 | 1.00 | 30.37 |
| ATOM | 26 | CG | GLU | 818 | 38.445 | 10.511 | 34.548 | 1.00 | 29.13 |
| ATOM | 27 | CD | GLU | 818 | 37.624 | 10.483 | 33.259 | 1.00 | 30.28 |
| ATOM | 28 | OE1 | GLU | 818 | 36.488 | 10.042 | 33.387 | 1.00 | 30.12 |
| ATOM | 29 | OE2 | GLU | 818 | 38.074 | 10.833 | 32.164 | 1.00 | 26.18 |
| ATOM | 30 | C | GLU | 818 | 41.046 | 13.383 | 34.440 | 1.00 | 31.56 |
| ATOM | 31 | O | GLU | 818 | 41.694 | 13.458 | 35.456 | 1.00 | 32.34 |
| ATOM | 32 | N | ARG | 819 | 41.592 | 13.683 | 33.265 | 1.00 | 32.84 |
| ATOM | 33 | H | ARG | 819 | 41.075 | 13.442 | 32.480 | 1.00 | 0.00 |
| ATOM | 34 | CA | ARG | 819 | 42.970 | 14.250 | 33.073 | 1.00 | 36.73 |
| ATOM | 35 | CB | ARG | 819 | 43.345 | 14.030 | 31.601 | 1.00 | 40.05 |
| ATOM | 36 | CG | ARG | 819 | 42.411 | 14.979 | 30.787 | 1.00 | 48.69 |
| ATOM | 37 | CD | ARG | 819 | 42.680 | 15.203 | 29.313 | 1.00 | 56.73 |
| ATOM | 38 | NE | ARG | 819 | 44.117 | 15.511 | 29.302 | 1.00 | 63.94 |
| ATOM | 39 | HE | ARG | 819 | 44.533 | 15.903 | 30.096 | 1.00 | 0.00 |
| ATOM | 40 | CZ | ARG | 819 | 44.918 | 15.183 | 28.286 | 1.00 | 68.24 |
| ATOM | 41 | NH1 | ARG | 819 | 44.444 | 14.542 | 27.196 | 1.00 | 70.53 |
| ATOM | 42 | HH11 | ARG | 819 | 43.481 | 14.290 | 27.119 | 1.00 | 0.00 |
| ATOM | 43 | HH12 | ARG | 819 | 45.067 | 14.352 | 26.436 | 1.00 | 0.00 |
| ATOM | 44 | NH2 | ARG | 819 | 46.236 | 15.449 | 28.404 | 1.00 | 69.91 |
| ATOM | 45 | HH21 | ARG | 819 | 46.601 | 15.855 | 29.241 | 1.00 | 0.00 |
| ATOM | 46 | HH22 | ARG | 819 | 46.853 | 15.254 | 27.641 | 1.00 | 0.00 |
| ATOM | 47 | C | ARG | 819 | 43.127 | 15.757 | 33.419 | 1.00 | 35.88 |
| ATOM | 48 | O | ARG | 819 | 44.225 | 16.293 | 33.596 | 1.00 | 35.29 |
| ATOM | 49 | N | LEU | 820 | 41.961 | 16.442 | 33.372 | 1.00 | 34.79 |
| ATOM | 50 | H | LEU | 820 | 41.176 | 15.881 | 33.255 | 1.00 | 0.00 |
| ATOM | 51 | CA | LEU | 820 | 41.729 | 17.890 | 33.600 | 1.00 | 32.61 |
| ATOM | 52 | CB | LEU | 820 | 40.197 | 18.133 | 33.352 | 1.00 | 32.03 |
| ATOM | 53 | CG | LEU | 820 | 39.756 | 19.085 | 32.197 | 1.00 | 32.13 |
| ATOM | 54 | CD1 | LEU | 820 | 40.822 | 19.079 | 31.067 | 1.00 | 32.44 |
| ATOM | 55 | CD2 | LEU | 820 | 38.311 | 18.713 | 31.772 | 1.00 | 28.77 |
| ATOM | 56 | C | LEU | 820 | 42.175 | 18.414 | 34.971 | 1.00 | 31.74 |
| ATOM | 57 | O | LEU | 820 | 42.013 | 17.736 | 35.995 | 1.00 | 30.49 |
| ATOM | 58 | N | PRO | 821 | 42.698 | 19.671 | 35.074 | 1.00 | 31.55 |
| ATOM | 59 | CD | PRO | 821 | 42.949 | 20.672 | 34.001 | 1.00 | 29.67 |
| ATOM | 60 | CA | PRO | 821 | 43.223 | 20.181 | 36.392 | 1.00 | 29.43 |
| ATOM | 61 | CB | PRO | 821 | 44.241 | 21.277 | 36.007 | 1.00 | 29.90 |
| ATOM | 62 | CG | PRO | 821 | 43.529 | 21.890 | 34.796 | 1.00 | 29.80 |
| ATOM | 63 | C | PRO | 821 | 42.175 | 20.715 | 37.378 | 1.00 | 27.63 |
| ATOM | 64 | O | PRO | 821 | 41.106 | 21.223 | 36.980 | 1.00 | 23.27 |
| ATOM | 65 | N | TYR | 822 | 42.529 | 20.609 | 38.664 | 1.00 | 24.48 |
| ATOM | 66 | H | TYR | 822 | 43.340 | 20.126 | 38.912 | 1.00 | 0.00 |
| ATOM | 67 | CA | TYR | 822 | 41.665 | 21.148 | 39.711 | 1.00 | 24.19 |
| ATOM | 68 | CB | TYR | 822 | 40.883 | 20.120 | 40.662 | 1.00 | 21.36 |
| ATOM | 69 | CG | TYR | 822 | 40.151 | 20.922 | 41.712 | 1.00 | 19.70 |
| ATOM | 70 | CD1 | TYR | 822 | 40.524 | 20.839 | 43.054 | 1.00 | 19.18 |
| ATOM | 71 | CE1 | TYR | 822 | 39.913 | 21.661 | 43.998 | 1.00 | 18.58 |
| ATOM | 72 | CD2 | TYR | 822 | 39.141 | 21.836 | 41.352 | 1.00 | 18.93 |
| ATOM | 73 | CE2 | TYR | 822 | 38.519 | 22.662 | 42.296 | 1.00 | 16.68 |
| ATOM | 74 | CZ | TYR | 822 | 38.912 | 22.569 | 43.618 | 1.00 | 17.30 |
| ATOM | 75 | OH | TYR | 822 | 38.263 | 23.303 | 44.570 | 1.00 | 16.64 |
| ATOM | 76 | HH | TYR | 822 | 38.640 | 23.157 | 45.440 | 1.00 | 0.00 |
| ATOM | 77 | C | TYR | 822 | 42.562 | 21.967 | 40.626 | 1.00 | 25.87 |
| ATOM | 78 | O | TYR | 822 | 43.266 | 21.477 | 41.505 | 1.00 | 26.77 |
| ATOM | 79 | N | ASP | 823 | 42.571 | 23.275 | 40.365 | 1.00 | 25.83 |
| ATOM | 80 | H | ASP | 823 | 42.032 | 23.554 | 39.594 | 1.00 | 0.00 |
| ATOM | 81 | CA | ASP | 823 | 43.337 | 24.279 | 41.147 | 1.00 | 24.29 |
| ATOM | 82 | CB | ASP | 823 | 43.543 | 25.511 | 40.253 | 1.00 | 23.76 |
| ATOM | 83 | CG | ASP | 823 | 44.450 | 26.525 | 40.913 | 1.00 | 26.80 |
| ATOM | 84 | OD1 | ASP | 823 | 44.772 | 26.387 | 42.082 | 1.00 | 26.12 |
| ATOM | 85 | OD2 | ASP | 823 | 44.853 | 27.476 | 40.264 | 1.00 | 27.77 |
| ATOM | 86 | C | ASP | 823 | 42.658 | 24.685 | 42.484 | 1.00 | 22.63 |
| ATOM | 87 | O | ASP | 823 | 41.982 | 25.714 | 42.611 | 1.00 | 21.21 |
| ATOM | 88 | N | ALA | 824 | 42.875 | 23.897 | 43.523 | 1.00 | 20.20 |
| ATOM | 89 | H | ALA | 824 | 43.376 | 23.061 | 43.373 | 1.00 | 0.00 |
| ATOM | 90 | CA | ALA | 824 | 42.270 | 24.195 | 44.820 | 1.00 | 20.96 |
| ATOM | 91 | CB | ALA | 824 | 42.717 | 23.188 | 45.879 | 1.00 | 21.34 |
| ATOM | 92 | C | ALA | 824 | 42.593 | 25.582 | 45.351 | 1.00 | 22.07 |
| ATOM | 93 | O | ALA | 824 | 41.750 | 26.208 | 45.998 | 1.00 | 21.64 |
| ATOM | 94 | N | SER | 825 | 43.796 | 26.128 | 45.119 | 1.00 | 23.32 |
| ATOM | 95 | H | SER | 825 | 44.491 | 25.667 | 44.610 | 1.00 | 0.00 |
| ATOM | 96 | CA | SER | 825 | 44.037 | 27.486 | 45.666 | 1.00 | 25.20 |
| ATOM | 97 | CB | SER | 825 | 45.481 | 28.013 | 45.487 | 1.00 | 26.75 |
| ATOM | 98 | OG | SER | 825 | 46.083 | 27.514 | 44.319 | 1.00 | 31.48 |
| ATOM | 99 | HG | SER | 825 | 47.001 | 27.805 | 44.289 | 1.00 | 0.00 |
| ATOM | 100 | C | SER | 825 | 43.111 | 28.487 | 45.014 | 1.00 | 25.68 |
| ATOM | 101 | O | SER | 825 | 42.763 | 29.491 | 45.661 | 1.00 | 26.86 |
| ATOM | 102 | N | LYS | 826 | 42.762 | 28.256 | 43.727 | 1.00 | 22.89 |
| ATOM | 103 | H | LYS | 826 | 43.088 | 27.455 | 43.271 | 1.00 | 0.00 |
| ATOM | 104 | CA | LYS | 826 | 41.801 | 29.182 | 43.099 | 1.00 | 20.36 |
| ATOM | 105 | CB | LYS | 826 | 41.937 | 29.153 | 41.553 | 1.00 | 17.34 |
| ATOM | 106 | CG | LYS | 826 | 40.966 | 30.128 | 40.888 | 1.00 | 15.86 |
| ATOM | 107 | CD | LYS | 826 | 41.348 | 30.362 | 39.448 | 1.00 | 19.71 |
| ATOM | 108 | CE | LYS | 826 | 40.198 | 31.175 | 38.751 | 1.00 | 22.52 |
| ATOM | 109 | NZ | LYS | 826 | 40.534 | 31.237 | 37.314 | 1.00 | 22.04 |
| ATOM | 110 | HZ1 | LYS | 826 | 40.855 | 30.298 | 37.002 | 1.00 | 0.00 |
| ATOM | 111 | HZ2 | LYS | 826 | 41.308 | 31.918 | 37.181 | 1.00 | 0.00 |
| ATOM | 112 | HZ3 | LYS | 826 | 39.708 | 31.517 | 36.753 | 1.00 | 0.00 |
| ATOM | 113 | C | LYS | 826 | 40.316 | 28.866 | 43.463 | 1.00 | 19.40 |
| ATOM | 114 | O | LYS | 826 | 39.517 | 29.739 | 43.791 | 1.00 | 20.66 |
| ATOM | 115 | N | TRP | 827 | 39.928 | 27.595 | 43.426 | 1.00 | 18.15 |
| ATOM | 116 | H | TRP | 827 | 40.604 | 26.912 | 43.257 | 1.00 | 0.00 |
| ATOM | 117 | CA | TRP | 827 | 38.516 | 27.235 | 43.615 | 1.00 | 15.11 |
| ATOM | 118 | CB | TRP | 827 | 38.158 | 26.134 | 42.628 | 1.00 | 12.70 |
| ATOM | 119 | CG | TRP | 827 | 38.404 | 26.654 | 41.302 | 1.00 | 11.62 |
| ATOM | 120 | CD2 | TRP | 827 | 37.578 | 27.598 | 40.649 | 1.00 | 13.45 |
| ATOM | 121 | CE2 | TRP | 827 | 38.180 | 27.822 | 39.388 | 1.00 | 14.06 |
| ATOM | 122 | CE3 | TRP | 827 | 36.409 | 28.273 | 41.028 | 1.00 | 11.48 |
| ATOM | 123 | CD1 | TRP | 827 | 39.487 | 26.354 | 40.437 | 1.00 | 11.34 |
| ATOM | 124 | NE1 | TRP | 827 | 39.329 | 27.044 | 39.277 | 1.00 | 12.70 |
| ATOM | 125 | HE1 | TRP | 827 | 39.837 | 26.953 | 38.448 | 1.00 | 0.00 |
| ATOM | 126 | CZ2 | TRP | 827 | 37.566 | 28.724 | 38.500 | 1.00 | 16.15 |
| ATOM | 127 | CZ3 | TRP | 827 | 35.815 | 29.152 | 40.157 | 1.00 | 13.29 |
| ATOM | 128 | CH2 | TRP | 827 | 36.395 | 29.381 | 38.891 | 1.00 | 15.01 |
| ATOM | 129 | C | TRP | 827 | 37.967 | 26.810 | 44.920 | 1.00 | 17.27 |
| ATOM | 130 | O | TRP | 827 | 36.755 | 26.918 | 45.168 | 1.00 | 13.03 |
| ATOM | 131 | N | GLU | 828 | 38.884 | 26.260 | 45.747 | 1.00 | 17.50 |
| ATOM | 132 | H | GLU | 828 | 39.843 | 26.330 | 45.571 | 1.00 | 0.00 |
| ATOM | 133 | CA | GLU | 828 | 38.371 | 25.708 | 46.993 | 1.00 | 18.24 |
| ATOM | 134 | CB | GLU | 828 | 39.570 | 24.935 | 47.609 | 1.00 | 17.73 |
| ATOM | 135 | CG | GLU | 828 | 39.131 | 24.056 | 48.815 | 1.00 | 17.12 |
| ATOM | 136 | CD | GLU | 828 | 38.501 | 22.730 | 48.444 | 1.00 | 20.98 |
| ATOM | 137 | OE1 | GLU | 828 | 37.834 | 22.140 | 49.311 | 1.00 | 16.67 |
| ATOM | 138 | OE2 | GLU | 828 | 38.705 | 22.294 | 47.313 | 1.00 | 20.29 |
| ATOM | 139 | C | GLU | 828 | 37.688 | 26.700 | 47.954 | 1.00 | 17.28 |
| ATOM | 140 | O | GLU | 828 | 38.158 | 27.784 | 48.259 | 1.00 | 19.57 |
| ATOM | 141 | N | PHE | 829 | 36.542 | 26.334 | 48.502 | 1.00 | 19.72 |
| ATOM | 142 | H | PHE | 829 | 36.192 | 25.459 | 48.243 | 1.00 | 0.00 |
| ATOM | 143 | CA | PHE | 829 | 35.771 | 27.191 | 49.424 | 1.00 | 18.22 |
| ATOM | 144 | CB | PHE | 829 | 34.411 | 27.604 | 48.733 | 1.00 | 17.89 |
| ATOM | 145 | CG | PHE | 829 | 33.672 | 28.638 | 49.511 | 1.00 | 16.83 |
| ATOM | 146 | CD1 | PHE | 829 | 34.186 | 29.937 | 49.585 | 1.00 | 15.75 |
| ATOM | 147 | CD2 | PHE | 829 | 32.436 | 28.316 | 50.127 | 1.00 | 17.29 |
| ATOM | 148 | CE1 | PHE | 829 | 33.443 | 30.925 | 50.270 | 1.00 | 17.58 |
| ATOM | 149 | CE2 | PHE | 829 | 31.704 | 29.314 | 50.802 | 1.00 | 19.65 |
| ATOM | 150 | CZ | PHE | 829 | 32.208 | 30.632 | 50.867 | 1.00 | 15.89 |
| ATOM | 151 | C | PHE | 829 | 35.498 | 26.463 | 50.727 | 1.00 | 19.05 |
| ATOM | 152 | O | PHE | 829 | 35.048 | 25.307 | 50.727 | 1.00 | 19.84 |
| ATOM | 153 | N | PRO | 830 | 35.687 | 27.106 | 51.908 | 1.00 | 22.25 |
| ATOM | 154 | CD | PRO | 830 | 36.115 | 28.538 | 52.128 | 1.00 | 20.70 |
| ATOM | 155 | CA | PRO | 830 | 35.415 | 26.394 | 53.193 | 1.00 | 21.70 |
| ATOM | 156 | CB | PRO | 830 | 35.849 | 27.416 | 54.269 | 1.00 | 18.92 |
| ATOM | 157 | CG | PRO | 830 | 36.752 | 28.379 | 53.507 | 1.00 | 20.16 |
| ATOM | 158 | C | PRO | 830 | 33.956 | 25.917 | 53.349 | 1.00 | 22.68 |
| ATOM | 159 | O | PRO | 830 | 33.009 | 26.707 | 53.284 | 1.00 | 22.31 |
| ATOM | 160 | N | ARG | 831 | 33.700 | 24.627 | 53.605 | 1.00 | 23.52 |
| ATOM | 161 | H | ARG | 831 | 34.417 | 23.983 | 53.617 | 1.00 | 0.00 |
| ATOM | 162 | CA | ARG | 831 | 32.269 | 24.189 | 53.759 | 1.00 | 27.42 |
| ATOM | 163 | CB | ARG | 831 | 32.089 | 22.583 | 53.987 | 1.00 | 28.25 |
| ATOM | 164 | CG | ARG | 831 | 31.701 | 21.656 | 52.722 | 1.00 | 34.10 |
| ATOM | 165 | CD | ARG | 831 | 31.739 | 20.035 | 52.494 | 1.00 | 31.31 |
| ATOM | 166 | NE | ARG | 831 | 33.057 | 19.515 | 52.881 | 1.00 | 34.55 |
| ATOM | 167 | HE | ARG | 831 | 33.277 | 19.699 | 53.821 | 1.00 | 0.00 |
| ATOM | 168 | CZ | ARG | 831 | 34.047 | 18.867 | 52.223 | 1.00 | 31.23 |
| ATOM | 169 | NH1 | ARG | 831 | 35.039 | 18.635 | 53.057 | 1.00 | 33.41 |
| ATOM | 170 | HH11 | ARG | 831 | 34.969 | 18.947 | 54.006 | 1.00 | 0.00 |
| ATOM | 171 | HH12 | ARG | 831 | 35.862 | 18.162 | 52.752 | 1.00 | 0.00 |
| ATOM | 172 | NH2 | ARG | 831 | 34.186 | 18.459 | 50.958 | 1.00 | 24.05 |
| ATOM | 173 | HH21 | ARG | 831 | 33.469 | 18.621 | 50.284 | 1.00 | 0.00 |
| ATOM | 174 | HH22 | ARG | 831 | 35.017 | 17.972 | 50.700 | 1.00 | 0.00 |
| ATOM | 175 | C | ARG | 831 | 31.630 | 24.922 | 54.937 | 1.00 | 25.82 |
| ATOM | 176 | O | ARG | 831 | 30.416 | 25.001 | 55.030 | 1.00 | 23.91 |
| ATOM | 177 | N | ASP | 832 | 32.425 | 25.411 | 55.885 | 1.00 | 26.79 |
| ATOM | 178 | H | ASP | 832 | 33.393 | 25.279 | 55.846 | 1.00 | 0.00 |
| ATOM | 179 | CA | ASP | 832 | 31.837 | 26.121 | 57.045 | 1.00 | 30.19 |
| ATOM | 180 | CB | ASP | 832 | 32.841 | 26.122 | 58.273 | 1.00 | 33.50 |
| ATOM | 181 | CG | ASP | 832 | 34.001 | 27.098 | 58.117 | 1.00 | 40.15 |
| ATOM | 182 | OD1 | ASP | 832 | 34.735 | 27.012 | 57.136 | 1.00 | 43.95 |
| ATOM | 183 | OD2 | ASP | 832 | 34.168 | 27.968 | 58.986 | 1.00 | 48.04 |
| ATOM | 184 | C | ASP | 832 | 31.403 | 27.552 | 56.699 | 1.00 | 28.37 |
| ATOM | 185 | O | ASP | 832 | 30.710 | 28.204 | 57.473 | 1.00 | 30.95 |
| ATOM | 186 | N | ARG | 833 | 31.909 | 28.132 | 55.603 | 1.00 | 26.68 |
| ATOM | 187 | H | ARG | 833 | 32.560 | 27.646 | 55.065 | 1.00 | 0.00 |
| ATOM | 188 | CA | ARG | 833 | 31.478 | 29.474 | 55.204 | 1.00 | 24.42 |
| ATOM | 189 | CB | ARG | 833 | 32.671 | 30.162 | 54.536 | 1.00 | 26.29 |
| ATOM | 190 | CG | ARG | 833 | 33.874 | 30.280 | 55.460 | 1.00 | 27.45 |
| ATOM | 191 | CD | ARG | 833 | 34.349 | 31.692 | 55.303 | 1.00 | 31.67 |
| ATOM | 192 | NE | ARG | 833 | 35.639 | 31.825 | 54.607 | 1.00 | 37.43 |
| ATOM | 193 | HE | ARG | 833 | 35.638 | 32.044 | 53.655 | 1.00 | 0.00 |
| ATOM | 194 | CZ | ARG | 833 | 36.848 | 31.584 | 55.157 | 1.00 | 37.26 |
| ATOM | 195 | NH1 | ARG | 833 | 37.997 | 31.764 | 54.464 | 1.00 | 38.46 |
| ATOM | 196 | HH11 | ARG | 833 | 37.957 | 32.084 | 53.517 | 1.00 | 0.00 |
| ATOM | 197 | HH12 | ARG | 833 | 38.887 | 31.592 | 54.884 | 1.00 | 0.00 |
| ATOM | 198 | NH2 | ARG | 833 | 36.947 | 31.065 | 56.365 | 1.00 | 38.42 |
| ATOM | 199 | HH21 | ARG | 833 | 36.133 | 30.840 | 56.896 | 1.00 | 0.00 |
| ATOM | 200 | HH22 | ARG | 833 | 37.859 | 30.898 | 56.741 | 1.00 | 0.00 |
| ATOM | 201 | C | ARG | 833 | 30.255 | 29.335 | 54.261 | 1.00 | 24.41 |
| ATOM | 202 | O | ARG | 833 | 29.783 | 30.294 | 53.654 | 1.00 | 23.74 |
| ATOM | 203 | N | LEU | 834 | 29.689 | 28.122 | 54.162 | 1.00 | 23.65 |
| ATOM | 204 | H | LEU | 834 | 30.062 | 27.388 | 54.691 | 1.00 | 0.00 |
| ATOM | 205 | CA | LEU | 834 | 28.534 | 27.802 | 53.327 | 1.00 | 23.26 |
| ATOM | 206 | CB | LEU | 834 | 28.984 | 26.739 | 52.322 | 1.00 | 22.96 |
| ATOM | 207 | CG | LEU | 834 | 28.110 | 26.348 | 51.123 | 1.00 | 23.99 |
| ATOM | 208 | CD1 | LEU | 834 | 27.990 | 27.467 | 50.036 | 1.00 | 20.59 |
| ATOM | 209 | CD2 | LEU | 834 | 28.817 | 25.129 | 50.477 | 1.00 | 23.64 |
| ATOM | 210 | C | LEU | 834 | 27.371 | 27.330 | 54.170 | 1.00 | 23.32 |
| ATOM | 211 | O | LEU | 834 | 27.447 | 26.447 | 55.016 | 1.00 | 24.87 |
| ATOM | 212 | N | LYS | 835 | 26.263 | 28.040 | 54.052 | 1.00 | 23.37 |
| ATOM | 213 | H | LYS | 835 | 26.314 | 28.854 | 53.518 | 1.00 | 0.00 |
| ATOM | 214 | CA | LYS | 835 | 25.047 | 27.686 | 54.787 | 1.00 | 22.24 |
| ATOM | 215 | CB | LYS | 835 | 24.381 | 28.891 | 55.552 | 1.00 | 21.82 |
| ATOM | 216 | C | LYS | 835 | 23.994 | 27.144 | 53.838 | 1.00 | 21.76 |
| ATOM | 217 | O | LYS | 835 | 23.333 | 27.869 | 53.102 | 1.00 | 20.81 |
| ATOM | 218 | N | LEU | 836 | 23.839 | 25.852 | 53.871 | 1.00 | 21.30 |
| ATOM | 219 | H | LEU | 836 | 24.445 | 25.329 | 54.424 | 1.00 | 0.00 |
| ATOM | 220 | CA | LEU | 836 | 22.859 | 25.163 | 53.059 | 1.00 | 23.25 |
| ATOM | 221 | CB | LEU | 836 | 23.169 | 23.700 | 53.045 | 1.00 | 23.47 |
| ATOM | 222 | CG | LEU | 836 | 24.388 | 23.235 | 52.215 | 1.00 | 24.57 |
| ATOM | 223 | CD1 | LEU | 836 | 25.676 | 23.971 | 52.512 | 1.00 | 26.18 |
| ATOM | 224 | CD2 | LEU | 836 | 24.582 | 21.764 | 52.594 | 1.00 | 25.31 |
| ATOM | 225 | C | LEU | 836 | 21.418 | 25.340 | 53.484 | 1.00 | 24.66 |
| ATOM | 226 | O | LEU | 836 | 21.053 | 25.185 | 54.657 | 1.00 | 26.55 |
| ATOM | 227 | N | GLY | 837 | 20.570 | 25.597 | 52.496 | 1.00 | 22.79 |
| ATOM | 228 | H | GLY | 837 | 20.952 | 25.699 | 51.600 | 1.00 | 0.00 |
| ATOM | 229 | CA | GLY | 837 | 19.119 | 25.787 | 52.696 | 1.00 | 21.51 |
| ATOM | 230 | C | GLY | 837 | 18.229 | 24.768 | 51.987 | 1.00 | 22.24 |
| ATOM | 231 | O | GLY | 837 | 18.444 | 23.553 | 51.988 | 1.00 | 22.82 |
| ATOM | 232 | N | LYS | 838 | 17.183 | 25.230 | 51.337 | 1.00 | 22.89 |
| ATOM | 233 | H | LYS | 838 | 17.144 | 26.203 | 51.322 | 1.00 | 0.00 |
| ATOM | 234 | CA | LYS | 838 | 16.196 | 24.416 | 50.611 | 1.00 | 23.66 |
| ATOM | 235 | CB | LYS | 838 | 15.012 | 25.355 | 50.412 | 1.00 | 30.94 |
| ATOM | 236 | CG | LYS | 838 | 13.664 | 24.735 | 50.776 | 1.00 | 41.94 |
| ATOM | 237 | CD | LYS | 838 | 12.797 | 24.558 | 49.463 | 1.00 | 48.64 |
| ATOM | 238 | CE | LYS | 838 | 11.473 | 23.720 | 49.617 | 1.00 | 52.11 |
| ATOM | 239 | NZ | LYS | 838 | 10.661 | 24.186 | 50.778 | 1.00 | 56.35 |
| ATOM | 240 | HZ1 | LYS | 838 | 10.610 | 25.227 | 50.796 | 1.00 | 0.00 |
| ATOM | 241 | HZ2 | LYS | 838 | 11.107 | 23.862 | 51.661 | 1.00 | 0.00 |
| ATOM | 242 | HZ3 | LYS | 838 | 9.697 | 23.802 | 50.714 | 1.00 | 0.00 |
| ATOM | 243 | C | LYS | 838 | 16.682 | 23.792 | 49.282 | 1.00 | 20.29 |
| ATOM | 244 | O | LYS | 838 | 17.493 | 24.382 | 48.575 | 1.00 | 18.42 |
| ATOM | 245 | N | PRO | 839 | 16.197 | 22.609 | 48.904 | 1.00 | 19.06 |
| ATOM | 246 | CD | PRO | 839 | 15.193 | 21.760 | 49.609 | 1.00 | 19.35 |
| ATOM | 247 | CA | PRO | 839 | 16.544 | 22.016 | 47.615 | 1.00 | 19.09 |
| ATOM | 248 | CB | PRO | 839 | 16.075 | 20.562 | 47.664 | 1.00 | 18.42 |
| ATOM | 249 | CG | PRO | 839 | 14.853 | 20.657 | 48.580 | 1.00 | 19.22 |
| ATOM | 250 | C | PRO | 839 | 15.874 | 22.769 | 46.458 | 1.00 | 20.31 |
| ATOM | 251 | O | PRO | 839 | 14.768 | 23.310 | 46.548 | 1.00 | 18.73 |
| ATOM | 252 | N | LEU | 840 | 16.661 | 22.957 | 45.421 | 1.00 | 18.44 |
| ATOM | 253 | H | LEU | 840 | 17.598 | 22.737 | 45.551 | 1.00 | 0.00 |
| ATOM | 254 | CA | LEU | 840 | 16.240 | 23.574 | 44.180 | 1.00 | 16.46 |
| ATOM | 255 | CB | LEU | 840 | 17.349 | 24.524 | 43.658 | 1.00 | 13.03 |
| ATOM | 256 | CG | LEU | 840 | 17.650 | 25.685 | 44.587 | 1.00 | 11.28 |
| ATOM | 257 | CD1 | LEU | 840 | 18.906 | 26.381 | 44.022 | 1.00 | 11.73 |
| ATOM | 258 | CD2 | LEU | 840 | 16.363 | 26.550 | 44.809 | 1.00 | 11.17 |
| ATOM | 259 | C | LEU | 840 | 15.975 | 22.417 | 43.199 | 1.00 | 17.90 |
| ATOM | 260 | O | LEU | 840 | 15.300 | 22.593 | 42.210 | 1.00 | 21.07 |
| ATOM | 261 | N | GLY | 841 | 16.481 | 21.212 | 43.359 | 1.00 | 16.05 |
| ATOM | 262 | H | GLY | 841 | 17.115 | 21.081 | 44.091 | 1.00 | 0.00 |
| ATOM | 263 | CA | GLY | 841 | 16.186 | 20.098 | 42.429 | 1.00 | 15.21 |
| ATOM | 264 | C | GLY | 841 | 16.768 | 18.804 | 42.976 | 1.00 | 17.35 |
| ATOM | 265 | O | GLY | 841 | 17.837 | 18.834 | 43.597 | 1.00 | 17.96 |
| ATOM | 266 | N | ARG | 842 | 16.067 | 17.695 | 42.958 | 1.00 | 17.95 |
| ATOM | 267 | H | ARG | 842 | 15.119 | 17.665 | 42.734 | 1.00 | 0.00 |
| ATOM | 268 | CA | ARG | 842 | 16.772 | 16.480 | 43.388 | 1.00 | 22.83 |
| ATOM | 269 | CB | ARG | 842 | 16.507 | 15.942 | 44.786 | 1.00 | 27.83 |
| ATOM | 270 | CG | ARG | 842 | 15.499 | 16.623 | 45.611 | 1.00 | 34.92 |
| ATOM | 271 | CD | ARG | 842 | 16.165 | 16.890 | 46.971 | 1.00 | 42.06 |
| ATOM | 272 | NE | ARG | 842 | 16.612 | 15.704 | 47.741 | 1.00 | 45.14 |
| ATOM | 273 | HE | ARG | 842 | 17.023 | 14.947 | 47.281 | 1.00 | 0.00 |
| ATOM | 274 | CZ | ARG | 842 | 16.406 | 15.611 | 49.075 | 1.00 | 49.30 |
| ATOM | 275 | NH1 | ARG | 842 | 15.741 | 16.567 | 49.798 | 1.00 | 48.29 |
| ATOM | 276 | HH11 | ARG | 842 | 15.352 | 17.369 | 49.345 | 1.00 | 0.00 |
| ATOM | 277 | HH12 | ARG | 842 | 15.635 | 16.456 | 50.789 | 1.00 | 0.00 |
| ATOM | 278 | NH2 | ARG | 842 | 16.961 | 14.562 | 49.720 | 1.00 | 49.71 |
| ATOM | 279 | HH21 | ARG | 842 | 17.496 | 13.883 | 49.222 | 1.00 | 0.00 |
| ATOM | 280 | HH22 | ARG | 842 | 16.846 | 14.467 | 50.711 | 1.00 | 0.00 |
| ATOM | 281 | C | ARG | 842 | 16.411 | 15.314 | 42.530 | 1.00 | 21.56 |
| ATOM | 282 | O | ARG | 842 | 15.302 | 15.255 | 42.013 | 1.00 | 23.71 |
| ATOM | 283 | N | GLY | 843 | 17.406 | 14.497 | 42.231 | 1.00 | 18.14 |
| ATOM | 284 | H | GLY | 843 | 18.277 | 14.674 | 42.644 | 1.00 | 0.00 |
| ATOM | 285 | CA | GLY | 843 | 17.215 | 13.298 | 41.430 | 1.00 | 18.57 |
| ATOM | 286 | C | GLY | 843 | 17.382 | 12.120 | 42.368 | 1.00 | 21.06 |
| ATOM | 287 | O | GLY | 843 | 17.094 | 12.230 | 43.576 | 1.00 | 20.61 |
| ATOM | 288 | N | ALA | 844 | 17.798 | 10.982 | 41.817 | 1.00 | 21.20 |
| ATOM | 289 | H | ALA | 844 | 17.998 | 10.955 | 40.858 | 1.00 | 0.00 |
| ATOM | 290 | CA | ALA | 844 | 17.994 | 9.777 | 42.688 | 1.00 | 24.78 |
| ATOM | 291 | CB | ALA | 844 | 17.997 | 8.438 | 41.847 | 1.00 | 23.33 |
| ATOM | 292 | C | ALA | 844 | 19.325 | 9.775 | 43.525 | 1.00 | 23.87 |
| ATOM | 293 | O | ALA | 844 | 19.442 | 9.311 | 44.671 | 1.00 | 25.72 |
| ATOM | 294 | N | PHE | 845 | 20.349 | 10.332 | 42.926 | 1.00 | 20.84 |
| ATOM | 295 | H | PHE | 845 | 20.083 | 10.740 | 42.087 | 1.00 | 0.00 |
| ATOM | 296 | CA | PHE | 845 | 21.683 | 10.386 | 43.509 | 1.00 | 19.75 |
| ATOM | 297 | CB | PHE | 845 | 22.646 | 9.685 | 42.555 | 1.00 | 21.12 |
| ATOM | 298 | CG | PHE | 845 | 22.231 | 8.322 | 42.237 | 1.00 | 23.20 |
| ATOM | 299 | CD1 | PHE | 845 | 22.348 | 7.911 | 40.917 | 1.00 | 24.90 |
| ATOM | 300 | CD2 | PHE | 845 | 21.757 | 7.457 | 43.232 | 1.00 | 27.51 |
| ATOM | 301 | CE1 | PHE | 845 | 21.984 | 6.604 | 40.589 | 1.00 | 29.51 |
| ATOM | 302 | CE2 | PHE | 845 | 21.386 | 6.135 | 42.914 | 1.00 | 30.76 |
| ATOM | 303 | CZ | PHE | 845 | 21.506 | 5.720 | 41.587 | 1.00 | 29.91 |
| ATOM | 304 | C | PHE | 845 | 22.207 | 11.777 | 43.801 | 1.00 | 19.67 |
| ATOM | 305 | O | PHE | 845 | 23.260 | 11.917 | 44.454 | 1.00 | 17.26 |
| ATOM | 306 | N | GLY | 846 | 21.583 | 12.805 | 43.221 | 1.00 | 15.44 |
| ATOM | 307 | H | GLY | 846 | 20.767 | 12.683 | 42.703 | 1.00 | 0.00 |
| ATOM | 308 | CA | GLY | 846 | 22.123 | 14.121 | 43.479 | 1.00 | 13.95 |
| ATOM | 309 | C | GLY | 846 | 21.099 | 15.146 | 43.783 | 1.00 | 13.84 |
| ATOM | 310 | O | GLY | 846 | 19.898 | 14.929 | 43.702 | 1.00 | 14.30 |
| ATOM | 311 | N | GLN | 847 | 21.611 | 16.312 | 44.057 | 1.00 | 12.69 |
| ATOM | 312 | H | GLN | 847 | 22.580 | 16.439 | 44.138 | 1.00 | 0.00 |
| ATOM | 313 | CA | GLN | 847 | 20.695 | 17.421 | 44.351 | 1.00 | 15.16 |
| ATOM | 314 | CB | GLN | 847 | 20.104 | 17.286 | 45.774 | 1.00 | 16.03 |
| ATOM | 315 | CG | GLN | 847 | 21.220 | 17.405 | 46.817 | 1.00 | 16.77 |
| ATOM | 316 | CD | GLN | 847 | 20.675 | 16.905 | 48.121 | 1.00 | 20.39 |
| ATOM | 317 | OE1 | GLN | 847 | 19.551 | 17.200 | 48.518 | 1.00 | 21.15 |
| ATOM | 318 | NE2 | GLN | 847 | 21.461 | 16.088 | 48.806 | 1.00 | 19.87 |
| ATOM | 319 | HE21 | GLN | 847 | 22.349 | 15.819 | 48.489 | 1.00 | 0.00 |
| ATOM | 320 | HE22 | GLN | 847 | 21.104 | 15.772 | 49.657 | 1.00 | 0.00 |
| ATOM | 321 | C | GLN | 847 | 21.365 | 18.773 | 44.249 | 1.00 | 12.74 |
| ATOM | 322 | O | GLN | 847 | 22.594 | 18.912 | 44.324 | 1.00 | 13.88 |
| ATOM | 323 | N | VAL | 848 | 20.561 | 19.756 | 43.925 | 1.00 | 12.69 |
| ATOM | 324 | H | VAL | 848 | 19.623 | 19.537 | 43.728 | 1.00 | 0.00 |
| ATOM | 325 | CA | VAL | 848 | 21.062 | 21.137 | 43.889 | 1.00 | 12.74 |
| ATOM | 326 | CB | VAL | 848 | 20.751 | 21.793 | 42.522 | 1.00 | 11.64 |
| ATOM | 327 | CG1 | VAL | 848 | 21.065 | 23.302 | 42.550 | 1.00 | 11.60 |
| ATOM | 328 | CG2 | VAL | 848 | 21.671 | 21.212 | 41.458 | 1.00 | 8.77 |
| ATOM | 329 | C | VAL | 848 | 20.305 | 21.832 | 45.061 | 1.00 | 14.00 |
| ATOM | 330 | O | VAL | 848 | 19.078 | 21.702 | 45.265 | 1.00 | 13.12 |
| ATOM | 331 | N | ILE | 849 | 21.033 | 22.512 | 45.928 | 1.00 | 15.33 |
| ATOM | 332 | H | ILE | 849 | 22.012 | 22.515 | 45.857 | 1.00 | 0.00 |
| ATOM | 333 | CA | ILE | 849 | 20.339 | 23.162 | 47.042 | 1.00 | 18.14 |
| ATOM | 334 | CB | ILE | 849 | 20.568 | 22.365 | 48.424 | 1.00 | 19.49 |
| ATOM | 335 | CG2 | ILE | 849 | 20.808 | 20.861 | 48.261 | 1.00 | 20.19 |
| ATOM | 336 | CG1 | ILE | 849 | 21.747 | 22.896 | 49.108 | 1.00 | 21.73 |
| ATOM | 337 | CD1 | ILE | 849 | 20.851 | 23.348 | 50.238 | 1.00 | 21.14 |
| ATOM | 338 | C | ILE | 849 | 20.774 | 24.584 | 47.158 | 1.00 | 16.43 |
| ATOM | 339 | O | ILE | 849 | 21.895 | 24.963 | 46.789 | 1.00 | 14.77 |
| ATOM | 340 | N | GLU | 850 | 19.809 | 25.415 | 47.482 | 1.00 | 16.82 |
| ATOM | 341 | H | GLU | 850 | 18.903 | 25.077 | 47.593 | 1.00 | 0.00 |
| ATOM | 342 | CA | GLU | 850 | 20.107 | 26.848 | 47.685 | 1.00 | 16.50 |
| ATOM | 343 | CB | GLU | 850 | 18.815 | 27.591 | 47.913 | 1.00 | 20.38 |
| ATOM | 344 | CG | GLU | 850 | 19.048 | 29.095 | 47.653 | 1.00 | 24.70 |
| ATOM | 345 | CD | GLU | 850 | 17.733 | 29.889 | 47.721 | 1.00 | 30.17 |
| ATOM | 346 | OE1 | GLU | 850 | 16.625 | 29.358 | 47.533 | 1.00 | 28.78 |
| ATOM | 347 | OE2 | GLU | 850 | 17.855 | 31.094 | 47.959 | 1.00 | 35.61 |
| ATOM | 348 | C | GLU | 850 | 21.015 | 27.090 | 48.878 | 1.00 | 16.75 |
| ATOM | 349 | O | GLU | 850 | 20.880 | 26.413 | 49.879 | 1.00 | 16.61 |
| ATOM | 350 | N | ALA | 851 | 21.924 | 28.019 | 48.837 | 1.00 | 16.42 |
| ATOM | 351 | H | ALA | 851 | 22.044 | 28.576 | 48.038 | 1.00 | 0.00 |
| ATOM | 352 | CA | ALA | 851 | 22.754 | 28.194 | 50.013 | 1.00 | 17.59 |
| ATOM | 353 | CB | ALA | 851 | 23.953 | 27.218 | 49.994 | 1.00 | 14.13 |
| ATOM | 354 | C | ALA | 851 | 23.294 | 29.579 | 50.069 | 1.00 | 17.78 |
| ATOM | 355 | O | ALA | 851 | 23.222 | 30.245 | 49.035 | 1.00 | 17.89 |
| ATOM | 356 | N | ASP | 852 | 23.728 | 30.075 | 51.243 | 1.00 | 18.59 |
| ATOM | 357 | H | ASP | 852 | 23.578 | 29.525 | 52.035 | 1.00 | 0.00 |
| ATOM | 358 | CA | ASP | 852 | 24.352 | 31.415 | 51.388 | 1.00 | 18.16 |
| ATOM | 359 | CB | ASP | 852 | 23.736 | 32.281 | 52.491 | 1.00 | 22.39 |
| ATOM | 360 | CG | ASP | 852 | 22.279 | 32.532 | 52.106 | 1.00 | 32.11 |
| ATOM | 361 | OD1 | ASP | 852 | 21.446 | 32.409 | 53.002 | 1.00 | 36.21 |
| ATOM | 362 | OD2 | ASP | 852 | 21.960 | 32.836 | 50.935 | 1.00 | 33.73 |
| ATOM | 363 | C | ASP | 852 | 25.777 | 31.226 | 51.730 | 1.00 | 17.74 |
| ATOM | 364 | O | ASP | 852 | 26.113 | 30.457 | 52.645 | 1.00 | 17.84 |
| ATOM | 365 | N | ALA | 853 | 26.605 | 31.794 | 50.885 | 1.00 | 17.04 |
| ATOM | 366 | H | ALA | 853 | 26.230 | 32.310 | 50.143 | 1.00 | 0.00 |
| ATOM | 367 | CA | ALA | 853 | 28.052 | 31.693 | 50.983 | 1.00 | 19.07 |
| ATOM | 368 | CB | ALA | 853 | 28.606 | 31.208 | 49.632 | 1.00 | 18.44 |
| ATOM | 369 | C | ALA | 853 | 28.722 | 33.012 | 51.369 | 1.00 | 20.45 |
| ATOM | 370 | O | ALA | 853 | 28.546 | 34.049 | 50.712 | 1.00 | 20.58 |
| ATOM | 371 | N | PHE | 854 | 29.504 | 33.003 | 52.458 | 1.00 | 21.45 |
| ATOM | 372 | H | PHE | 854 | 29.639 | 32.140 | 52.909 | 1.00 | 0.00 |
| ATOM | 373 | CA | PHE | 854 | 30.191 | 34.229 | 52.941 | 1.00 | 19.79 |
| ATOM | 374 | CB | PHE | 854 | 30.454 | 34.114 | 54.487 | 1.00 | 18.52 |
| ATOM | 375 | CG | PHE | 854 | 30.901 | 35.415 | 55.055 | 1.00 | 17.17 |
| ATOM | 376 | CD1 | PHE | 854 | 32.273 | 35.691 | 55.282 | 1.00 | 18.69 |
| ATOM | 377 | CD2 | PHE | 854 | 29.935 | 36.370 | 55.387 | 1.00 | 15.05 |
| ATOM | 378 | CE1 | PHE | 854 | 32.693 | 36.920 | 55.852 | 1.00 | 15.00 |
| ATOM | 379 | CE2 | PHE | 854 | 30.345 | 37.575 | 55.953 | 1.00 | 15.41 |
| ATOM | 380 | CZ | PHE | 854 | 31.697 | 37.841 | 56.183 | 1.00 | 13.10 |
| ATOM | 381 | C | PHE | 854 | 31.464 | 34.562 | 52.227 | 1.00 | 18.99 |
| ATOM | 382 | O | PHE | 854 | 32.466 | 33.880 | 52.388 | 1.00 | 21.10 |
| ATOM | 383 | N | GLY | 855 | 31.447 | 35.555 | 51.393 | 1.00 | 17.85 |
| ATOM | 384 | H | GLY | 855 | 30.599 | 36.010 | 51.217 | 1.00 | 0.00 |
| ATOM | 385 | CA | GLY | 855 | 32.628 | 35.987 | 50.671 | 1.00 | 18.86 |
| ATOM | 386 | C | GLY | 855 | 33.078 | 35.062 | 49.561 | 1.00 | 21.52 |
| ATOM | 387 | O | GLY | 855 | 34.246 | 35.116 | 49.199 | 1.00 | 23.50 |
| ATOM | 388 | N | ILE | 856 | 32.219 | 34.235 | 48.968 | 1.00 | 19.73 |
| ATOM | 389 | H | ILE | 856 | 31.292 | 34.211 | 49.274 | 1.00 | 0.00 |
| ATOM | 390 | CA | ILE | 856 | 32.757 | 33.376 | 47.897 | 1.00 | 19.84 |
| ATOM | 391 | CB | ILE | 856 | 31.626 | 32.339 | 47.482 | 1.00 | 18.67 |
| ATOM | 392 | CG2 | ILE | 856 | 30.428 | 33.051 | 46.830 | 1.00 | 14.03 |
| ATOM | 393 | CG1 | ILE | 856 | 32.290 | 31.254 | 46.592 | 1.00 | 16.31 |
| ATOM | 394 | CD1 | ILE | 856 | 31.413 | 30.021 | 46.571 | 1.00 | 15.88 |
| ATOM | 395 | C | ILE | 856 | 33.278 | 34.191 | 46.683 | 1.00 | 20.90 |
| ATOM | 396 | O | ILE | 856 | 34.277 | 33.821 | 46.094 | 1.00 | 21.85 |
| ATOM | 397 | N | ASP | 857 | 32.704 | 35.319 | 46.318 | 1.00 | 21.16 |
| ATOM | 398 | H | ASP | 857 | 31.943 | 35.625 | 46.850 | 1.00 | 0.00 |
| ATOM | 399 | CA | ASP | 857 | 33.196 | 36.055 | 45.157 | 1.00 | 26.29 |
| ATOM | 400 | CB | ASP | 857 | 32.161 | 35.926 | 43.956 | 1.00 | 28.40 |
| ATOM | 401 | CG | ASP | 857 | 30.683 | 36.381 | 44.188 | 1.00 | 33.97 |
| ATOM | 402 | OD1 | ASP | 857 | 30.394 | 37.101 | 45.174 | 1.00 | 33.50 |
| ATOM | 403 | OD2 | ASP | 857 | 29.829 | 36.033 | 43.339 | 1.00 | 30.42 |
| ATOM | 404 | C | ASP | 857 | 33.474 | 37.515 | 45.473 | 1.00 | 29.12 |
| ATOM | 405 | O | ASP | 857 | 34.114 | 38.259 | 44.721 | 1.00 | 31.62 |
| ATOM | 406 | N | LYS | 858 | 33.004 | 37.996 | 46.613 | 1.00 | 31.16 |
| ATOM | 407 | H | LYS | 858 | 32.504 | 37.398 | 47.206 | 1.00 | 0.00 |
| ATOM | 408 | CA | LYS | 858 | 33.275 | 39.410 | 46.974 | 1.00 | 32.72 |
| ATOM | 409 | CB | LYS | 858 | 32.055 | 40.296 | 46.735 | 1.00 | 35.69 |
| ATOM | 410 | CG | LYS | 858 | 32.417 | 41.779 | 46.544 | 1.00 | 35.38 |
| ATOM | 411 | CD | LYS | 858 | 31.123 | 42.623 | 46.356 | 1.00 | 34.63 |
| ATOM | 412 | CE | LYS | 858 | 31.428 | 44.103 | 46.652 | 1.00 | 35.00 |
| ATOM | 413 | NZ | LYS | 858 | 30.169 | 44.882 | 46.616 | 1.00 | 39.84 |
| ATOM | 414 | HZ1 | LYS | 858 | 29.428 | 44.390 | 47.151 | 1.00 | 0.00 |
| ATOM | 415 | HZ2 | LYS | 858 | 29.874 | 45.012 | 45.629 | 1.00 | 0.00 |
| ATOM | 416 | HZ3 | LYS | 858 | 30.349 | 45.814 | 47.042 | 1.00 | 0.00 |
| ATOM | 417 | C | LYS | 858 | 33.613 | 39.420 | 48.434 | 1.00 | 31.55 |
| ATOM | 418 | O | LYS | 858 | 32.873 | 38.955 | 49.308 | 1.00 | 30.29 |
| ATOM | 419 | N | THR | 859 | 34.772 | 39.936 | 48.689 | 1.00 | 29.77 |
| ATOM | 420 | H | THR | 859 | 35.305 | 40.299 | 47.964 | 1.00 | 0.00 |
| ATOM | 421 | CA | THR | 859 | 35.283 | 39.977 | 50.067 | 1.00 | 31.19 |
| ATOM | 422 | CB | THR | 859 | 36.556 | 40.909 | 50.214 | 1.00 | 31.81 |
| ATOM | 423 | OG1 | THR | 859 | 37.603 | 40.263 | 49.472 | 1.00 | 34.51 |
| ATOM | 424 | HG1 | THR | 859 | 38.431 | 40.731 | 49.604 | 1.00 | 0.00 |
| ATOM | 425 | CG2 | THR | 859 | 37.045 | 41.169 | 51.688 | 1.00 | 31.36 |
| ATOM | 426 | C | THR | 859 | 34.256 | 40.413 | 51.096 | 1.00 | 27.55 |
| ATOM | 427 | O | THR | 859 | 33.614 | 41.449 | 50.988 | 1.00 | 27.92 |
| ATOM | 428 | N | ALA | 860 | 34.052 | 39.485 | 52.000 | 1.00 | 24.95 |
| ATOM | 429 | H | ALA | 860 | 34.374 | 38.613 | 51.707 | 1.00 | 0.00 |
| ATOM | 430 | CA | ALA | 860 | 33.192 | 39.588 | 53.148 | 1.00 | 23.38 |
| ATOM | 431 | CB | ALA | 860 | 33.821 | 40.653 | 54.084 | 1.00 | 18.54 |
| ATOM | 432 | C | ALA | 860 | 31.723 | 39.911 | 52.819 | 1.00 | 22.00 |
| ATOM | 433 | O | ALA | 860 | 31.034 | 40.494 | 53.660 | 1.00 | 20.49 |
| ATOM | 434 | N | THR | 861 | 31.197 | 39.527 | 51.636 | 1.00 | 22.19 |
| ATOM | 435 | H | THR | 861 | 31.779 | 39.138 | 50.948 | 1.00 | 0.00 |
| ATOM | 436 | CA | THR | 861 | 29.756 | 39.789 | 51.375 | 1.00 | 22.93 |
| ATOM | 437 | CB | THR | 861 | 29.509 | 40.916 | 50.204 | 1.00 | 22.98 |
| ATOM | 438 | OG1 | THR | 861 | 28.738 | 40.433 | 49.102 | 1.00 | 25.58 |
| ATOM | 439 | HG1 | THR | 861 | 28.558 | 41.176 | 48.510 | 1.00 | 0.00 |
| ATOM | 440 | CG2 | THR | 861 | 30.839 | 41.510 | 49.784 | 1.00 | 16.62 |
| ATOM | 441 | C | THR | 861 | 29.008 | 38.491 | 51.101 | 1.00 | 23.22 |
| ATOM | 442 | O | THR | 861 | 29.525 | 37.569 | 50.448 | 1.00 | 22.80 |
| ATOM | 443 | N | CYS | 862 | 27.997 | 38.312 | 51.955 | 1.00 | 22.26 |
| ATOM | 444 | H | CYS | 862 | 27.808 | 39.055 | 52.577 | 1.00 | 0.00 |
| ATOM | 445 | CA | CYS | 862 | 27.090 | 37.138 | 51.951 | 1.00 | 26.89 |
| ATOM | 446 | CB | CYS | 862 | 26.068 | 37.272 | 53.173 | 1.00 | 30.00 |
| ATOM | 447 | SG | CYS | 862 | 26.704 | 37.950 | 54.931 | 1.00 | 50.72 |
| ATOM | 448 | C | CYS | 862 | 26.414 | 37.082 | 50.536 | 1.00 | 27.13 |
| ATOM | 449 | O | CYS | 862 | 25.940 | 38.058 | 49.965 | 1.00 | 24.57 |
| ATOM | 450 | N | ARG | 863 | 26.497 | 35.933 | 49.842 | 1.00 | 25.74 |
| ATOM | 451 | H | ARG | 863 | 27.023 | 35.206 | 50.233 | 1.00 | 0.00 |
| ATOM | 452 | CA | ARG | 863 | 25.955 | 35.767 | 48.466 | 1.00 | 23.72 |
| ATOM | 453 | CB | ARG | 863 | 27.245 | 35.894 | 47.619 | 1.00 | 26.40 |
| ATOM | 454 | CG | ARG | 863 | 27.500 | 35.188 | 46.323 | 1.00 | 31.17 |
| ATOM | 455 | CD | ARG | 863 | 26.524 | 35.672 | 45.312 | 1.00 | 32.88 |
| ATOM | 456 | NE | ARG | 863 | 27.135 | 35.612 | 43.963 | 1.00 | 39.60 |
| ATOM | 457 | HE | ARG | 863 | 27.927 | 36.156 | 43.816 | 1.00 | 0.00 |
| ATOM | 458 | CZ | ARG | 863 | 26.614 | 34.982 | 42.872 | 1.00 | 39.23 |
| ATOM | 459 | NH1 | ARG | 863 | 27.351 | 35.103 | 41.734 | 1.00 | 33.37 |
| ATOM | 460 | HH11 | ARG | 863 | 28.205 | 35.627 | 41.731 | 1.00 | 0.00 |
| ATOM | 461 | HH12 | ARG | 863 | 27.041 | 34.659 | 40.891 | 1.00 | 0.00 |
| ATOM | 462 | NH2 | ARG | 863 | 25.431 | 34.230 | 42.916 | 1.00 | 38.68 |
| ATOM | 463 | HH21 | ARG | 863 | 24.927 | 34.135 | 43.772 | 1.00 | 0.00 |
| ATOM | 464 | HH22 | ARG | 863 | 25.105 | 33.782 | 42.085 | 1.00 | 0.00 |
| ATOM | 465 | C | ARG | 863 | 25.164 | 34.484 | 48.284 | 1.00 | 21.34 |
| ATOM | 466 | O | ARG | 863 | 25.633 | 33.414 | 48.669 | 1.00 | 21.78 |
| ATOM | 467 | N | THR | 864 | 23.890 | 34.527 | 47.859 | 1.00 | 20.17 |
| ATOM | 468 | H | THR | 864 | 23.437 | 35.393 | 47.759 | 1.00 | 0.00 |
| ATOM | 469 | CA | THR | 864 | 23.159 | 33.267 | 47.620 | 1.00 | 17.79 |
| ATOM | 470 | CB | THR | 864 | 21.687 | 33.493 | 47.383 | 1.00 | 19.29 |
| ATOM | 471 | OG1 | THR | 864 | 21.195 | 34.166 | 48.528 | 1.00 | 21.36 |
| ATOM | 472 | HG1 | THR | 864 | 20.338 | 34.563 | 48.374 | 1.00 | 0.00 |
| ATOM | 473 | CG2 | THR | 864 | 20.915 | 32.185 | 47.259 | 1.00 | 16.58 |
| ATOM | 474 | C | THR | 864 | 23.713 | 32.599 | 46.390 | 1.00 | 15.94 |
| ATOM | 475 | O | THR | 864 | 23.905 | 33.177 | 45.316 | 1.00 | 15.24 |
| ATOM | 476 | N | VAL | 865 | 23.911 | 31.316 | 46.521 | 1.00 | 14.99 |
| ATOM | 477 | H | VAL | 865 | 23.602 | 30.903 | 47.352 | 1.00 | 0.00 |
| ATOM | 478 | CA | VAL | 865 | 24.486 | 30.426 | 45.481 | 1.00 | 13.50 |
| ATOM | 479 | CB | VAL | 865 | 26.000 | 30.130 | 45.838 | 1.00 | 10.39 |
| ATOM | 480 | CG1 | VAL | 865 | 26.788 | 31.429 | 45.847 | 1.00 | 11.04 |
| ATOM | 481 | CG2 | VAL | 865 | 26.100 | 29.344 | 47.188 | 1.00 | 7.95 |
| ATOM | 482 | C | VAL | 865 | 23.669 | 29.091 | 45.379 | 1.00 | 11.74 |
| ATOM | 483 | O | VAL | 865 | 22.748 | 28.812 | 46.183 | 1.00 | 12.93 |
| ATOM | 484 | N | ALA | 866 | 23.923 | 28.336 | 44.264 | 1.00 | 11.20 |
| ATOM | 485 | H | ALA | 866 | 24.534 | 28.705 | 43.584 | 1.00 | 0.00 |
| ATOM | 486 | CA | ALA | 866 | 23.304 | 27.003 | 44.043 | 1.00 | 10.46 |
| ATOM | 487 | CB | ALA | 866 | 22.635 | 26.855 | 42.636 | 1.00 | 9.79 |
| ATOM | 488 | C | ALA | 866 | 24.423 | 25.956 | 44.118 | 1.00 | 10.05 |
| ATOM | 489 | O | ALA | 866 | 25.478 | 26.079 | 43.494 | 1.00 | 11.28 |
| ATOM | 490 | N | VAL | 867 | 24.295 | 24.955 | 44.978 | 1.00 | 11.68 |
| ATOM | 491 | H | VAL | 867 | 23.505 | 24.941 | 45.558 | 1.00 | 0.00 |
| ATOM | 492 | CA | VAL | 867 | 25.335 | 23.906 | 45.093 | 1.00 | 13.68 |
| ATOM | 493 | CB | VAL | 867 | 26.187 | 23.941 | 46.529 | 1.00 | 14.58 |
| ATOM | 494 | CG1 | VAL | 867 | 25.677 | 25.078 | 47.410 | 1.00 | 12.82 |
| ATOM | 495 | CG2 | VAL | 867 | 26.268 | 22.606 | 47.214 | 1.00 | 11.71 |
| ATOM | 496 | C | VAL | 867 | 24.818 | 22.523 | 44.836 | 1.00 | 10.77 |
| ATOM | 497 | O | VAL | 867 | 23.735 | 22.091 | 45.238 | 1.00 | 11.68 |
| ATOM | 498 | N | LYS | 868 | 25.552 | 21.899 | 43.957 | 1.00 | 10.46 |
| ATOM | 499 | H | LYS | 868 | 26.304 | 22.372 | 43.555 | 1.00 | 0.00 |
| ATOM | 500 | CA | LYS | 868 | 25.233 | 20.530 | 43.580 | 1.00 | 9.73 |
| ATOM | 501 | CB | LYS | 868 | 25.506 | 20.278 | 42.083 | 1.00 | 9.74 |
| ATOM | 502 | CG | LYS | 868 | 25.013 | 18.911 | 41.565 | 1.00 | 9.56 |
| ATOM | 503 | CD | LYS | 868 | 25.214 | 18.690 | 40.079 | 1.00 | 8.12 |
| ATOM | 504 | CE | LYS | 868 | 24.352 | 19.657 | 39.220 | 1.00 | 8.25 |
| ATOM | 505 | NZ | LYS | 868 | 24.647 | 19.271 | 37.835 | 1.00 | 7.26 |
| ATOM | 506 | HZ1 | LYS | 868 | 24.638 | 18.234 | 37.749 | 1.00 | 0.00 |
| ATOM | 507 | HZ2 | LYS | 868 | 25.575 | 19.642 | 37.558 | 1.00 | 0.00 |
| ATOM | 508 | HZ3 | LYS | 868 | 23.918 | 19.673 | 37.218 | 1.00 | 0.00 |
| ATOM | 509 | C | LYS | 868 | 26.130 | 19.626 | 44.417 | 1.00 | 11.25 |
| ATOM | 510 | O | LYS | 868 | 27.317 | 19.845 | 44.606 | 1.00 | 9.19 |
| ATOM | 511 | N | MET | 869 | 25.533 | 18.565 | 44.911 | 1.00 | 12.26 |
| ATOM | 512 | H | MET | 869 | 24.563 | 18.486 | 44.765 | 1.00 | 0.00 |
| ATOM | 513 | CA | MET | 869 | 26.231 | 17.547 | 45.745 | 1.00 | 13.48 |
| ATOM | 514 | CB | MET | 869 | 26.340 | 18.062 | 47.246 | 1.00 | 11.40 |
| ATOM | 515 | CG | MET | 869 | 24.933 | 18.232 | 47.850 | 1.00 | 18.20 |
| ATOM | 516 | SD | MET | 869 | 24.842 | 19.218 | 49.379 | 1.00 | 25.16 |
| ATOM | 517 | CE | MET | 869 | 25.599 | 17.880 | 50.338 | 1.00 | 28.52 |
| ATOM | 518 | C | MET | 869 | 25.433 | 16.265 | 45.666 | 1.00 | 11.36 |
| ATOM | 519 | O | MET | 869 | 24.323 | 16.213 | 45.138 | 1.00 | 10.82 |
| ATOM | 520 | N | LEU | 870 | 26.003 | 15.185 | 46.140 | 1.00 | 14.25 |
| ATOM | 521 | H | LEU | 870 | 26.927 | 15.252 | 46.463 | 1.00 | 0.00 |
| ATOM | 522 | CA | LEU | 870 | 25.343 | 13.884 | 46.151 | 1.00 | 15.03 |
| ATOM | 523 | CB | LEU | 870 | 26.432 | 12.769 | 46.150 | 1.00 | 12.93 |
| ATOM | 524 | CG | LEU | 870 | 27.410 | 12.843 | 44.905 | 1.00 | 14.77 |
| ATOM | 525 | CD1 | LEU | 870 | 28.334 | 11.598 | 44.943 | 1.00 | 14.88 |
| ATOM | 526 | CD2 | LEU | 870 | 26.661 | 12.776 | 43.531 | 1.00 | 14.60 |
| ATOM | 527 | C | LEU | 870 | 24.350 | 13.695 | 47.283 | 1.00 | 16.21 |
| ATOM | 528 | O | LEU | 870 | 24.255 | 14.434 | 48.239 | 1.00 | 17.66 |
| ATOM | 529 | N | LYS | 871 | 23.606 | 12.652 | 47.199 | 1.00 | 16.93 |
| ATOM | 530 | H | LYS | 871 | 23.758 | 12.082 | 46.413 | 1.00 | 0.00 |
| ATOM | 531 | CA | LYS | 871 | 22.544 | 12.268 | 48.111 | 1.00 | 20.39 |
| ATOM | 532 | CB | LYS | 871 | 21.196 | 12.248 | 47.362 | 1.00 | 21.86 |
| ATOM | 533 | CG | LYS | 871 | 20.402 | 13.470 | 47.378 | 1.00 | 24.33 |
| ATOM | 534 | CD | LYS | 871 | 19.311 | 13.174 | 46.359 | 1.00 | 28.33 |
| ATOM | 535 | CE | LYS | 871 | 18.025 | 12.602 | 46.953 | 1.00 | 31.37 |
| ATOM | 536 | NZ | LYS | 871 | 18.088 | 11.137 | 47.091 | 1.00 | 38.80 |
| ATOM | 537 | HZ1 | LYS | 871 | 18.943 | 10.843 | 47.601 | 1.00 | 0.00 |
| ATOM | 538 | HZ2 | LYS | 871 | 18.053 | 10.683 | 46.153 | 1.00 | 0.00 |
| ATOM | 539 | HZ3 | LYS | 871 | 17.246 | 10.825 | 47.622 | 1.00 | 0.00 |
| ATOM | 540 | C | LYS | 871 | 22.759 | 10.838 | 48.575 | 1.00 | 19.14 |
| ATOM | 541 | O | LYS | 871 | 23.414 | 10.036 | 47.904 | 1.00 | 19.15 |
| ATOM | 542 | N | GLU | 872 | 22.130 | 10.457 | 49.652 | 1.00 | 20.40 |
| ATOM | 543 | H | GLU | 872 | 21.701 | 11.111 | 50.216 | 1.00 | 0.00 |
| ATOM | 544 | CA | GLU | 872 | 22.197 | 9.071 | 50.122 | 1.00 | 22.02 |
| ATOM | 545 | CB | GLU | 872 | 21.308 | 8.922 | 51.374 | 1.00 | 26.62 |
| ATOM | 546 | CG | GLU | 872 | 21.094 | 7.468 | 51.885 | 1.00 | 36.24 |
| ATOM | 547 | CD | GLU | 872 | 22.398 | 6.636 | 52.153 | 1.00 | 41.04 |
| ATOM | 548 | OE1 | GLU | 872 | 22.305 | 5.403 | 51.961 | 1.00 | 43.51 |
| ATOM | 549 | OE2 | GLU | 872 | 23.460 | 7.199 | 52.535 | 1.00 | 43.05 |
| ATOM | 550 | C | GLU | 872 | 21.739 | 8.205 | 48.973 | 1.00 | 20.27 |
| ATOM | 551 | O | GLU | 872 | 20.729 | 8.484 | 48.322 | 1.00 | 23.79 |
| ATOM | 552 | N | GLY | 873 | 22.466 | 7.165 | 48.677 | 1.00 | 18.69 |
| ATOM | 553 | H | GLY | 873 | 23.244 | 6.974 | 49.230 | 1.00 | 0.00 |
| ATOM | 554 | CA | GLY | 873 | 22.101 | 6.313 | 47.565 | 1.00 | 18.51 |
| ATOM | 555 | C | GLY | 873 | 23.144 | 6.357 | 46.488 | 1.00 | 18.79 |
| ATOM | 556 | O | GLY | 873 | 23.354 | 5.409 | 45.727 | 1.00 | 20.59 |
| ATOM | 557 | N | ALA | 874 | 23.791 | 7.499 | 46.343 | 1.00 | 17.64 |
| ATOM | 558 | H | ALA | 874 | 23.532 | 8.272 | 46.896 | 1.00 | 0.00 |
| ATOM | 559 | CA | ALA | 874 | 24.849 | 7.606 | 45.324 | 1.00 | 16.20 |
| ATOM | 560 | CB | ALA | 874 | 25.326 | 9.061 | 45.225 | 1.00 | 15.41 |
| ATOM | 561 | C | ALA | 874 | 26.050 | 6.715 | 45.764 | 1.00 | 17.34 |
| ATOM | 562 | O | ALA | 874 | 26.282 | 6.355 | 46.929 | 1.00 | 16.59 |
| ATOM | 563 | N | THR | 875 | 26.952 | 6.477 | 44.834 | 1.00 | 17.68 |
| ATOM | 564 | H | THR | 875 | 26.812 | 6.898 | 43.971 | 1.00 | 0.00 |
| ATOM | 565 | CA | THR | 875 | 28.133 | 5.647 | 45.031 | 1.00 | 15.86 |
| ATOM | 566 | CB | THR | 875 | 28.015 | 4.342 | 44.156 | 1.00 | 18.80 |
| ATOM | 567 | OG1 | THR | 875 | 28.144 | 4.721 | 42.783 | 1.00 | 16.27 |
| ATOM | 568 | HG1 | THR | 875 | 27.829 | 4.041 | 42.177 | 1.00 | 0.00 |
| ATOM | 569 | CG2 | THR | 875 | 26.695 | 3.590 | 44.364 | 1.00 | 17.75 |
| ATOM | 570 | C | THR | 875 | 29.324 | 6.455 | 44.587 | 1.00 | 15.37 |
| ATOM | 571 | O | THR | 875 | 29.233 | 7.548 | 44.027 | 1.00 | 14.44 |
| ATOM | 572 | N | HIS | 876 | 30.505 | 5.918 | 44.736 | 1.00 | 15.83 |
| ATOM | 573 | H | HIS | 876 | 30.605 | 5.070 | 45.195 | 1.00 | 0.00 |
| ATOM | 574 | CA | HIS | 876 | 31.661 | 6.667 | 44.306 | 1.00 | 16.35 |
| ATOM | 575 | C | HIS | 876 | 31.681 | 6.958 | 42.802 | 1.00 | 16.30 |
| ATOM | 576 | O | HIS | 876 | 32.330 | 7.897 | 42.338 | 1.00 | 17.06 |
| ATOM | 577 | CB | HIS | 876 | 32.881 | 5.904 | 44.704 | 1.00 | 17.01 |
| ATOM | 578 | CG | HIS | 876 | 34.046 | 6.698 | 44.285 | 1.00 | 19.06 |
| ATOM | 579 | ND1 | HIS | 876 | 34.396 | 7.909 | 44.805 | 1.00 | 15.71 |
| ATOM | 580 | CE1 | HIS | 876 | 35.537 | 8.294 | 44.188 | 1.00 | 14.98 |
| ATOM | 581 | CD2 | HIS | 876 | 34.976 | 6.311 | 43.316 | 1.00 | 19.82 |
| ATOM | 582 | NE2 | HIS | 876 | 35.888 | 7.314 | 43.285 | 1.00 | 22.06 |
| ATOM | 583 | HE2 | HIS | 876 | 36.689 | 7.303 | 42.742 | 1.00 | 0.00 |
| ATOM | 584 | N | SER | 877 | 30.994 | 6.206 | 41.955 | 1.00 | 15.61 |
| ATOM | 585 | H | SER | 877 | 30.523 | 5.410 | 42.282 | 1.00 | 0.00 |
| ATOM | 586 | CA | SER | 877 | 31.015 | 6.501 | 40.507 | 1.00 | 15.13 |
| ATOM | 587 | CB | SER | 877 | 30.430 | 5.284 | 39.718 | 1.00 | 18.01 |
| ATOM | 588 | OG | SER | 877 | 29.026 | 5.340 | 39.627 | 1.00 | 27.09 |
| ATOM | 589 | HG | SER | 877 | 28.734 | 4.429 | 39.479 | 1.00 | 0.00 |
| ATOM | 590 | C | SER | 877 | 30.263 | 7.798 | 40.276 | 1.00 | 13.38 |
| ATOM | 591 | O | SER | 877 | 30.693 | 8.601 | 39.469 | 1.00 | 12.03 |
| ATOM | 592 | N | GLU | 878 | 29.199 | 8.116 | 41.017 | 1.00 | 13.81 |
| ATOM | 593 | H | GLU | 878 | 28.893 | 7.434 | 41.651 | 1.00 | 0.00 |
| ATOM | 594 | CA | GLU | 878 | 28.482 | 9.422 | 40.898 | 1.00 | 13.30 |
| ATOM | 595 | CB | GLU | 878 | 27.198 | 9.460 | 41.694 | 1.00 | 11.59 |
| ATOM | 596 | CG | GLU | 878 | 26.034 | 8.820 | 40.886 | 1.00 | 16.46 |
| ATOM | 597 | CD | GLU | 878 | 26.107 | 7.294 | 40.943 | 1.00 | 21.54 |
| ATOM | 598 | OE1 | GLU | 878 | 26.374 | 6.726 | 42.002 | 1.00 | 22.59 |
| ATOM | 599 | OE2 | GLU | 878 | 25.896 | 6.638 | 39.940 | 1.00 | 21.48 |
| ATOM | 600 | C | GLU | 878 | 29.360 | 10.539 | 41.409 | 1.00 | 14.61 |
| ATOM | 601 | O | GLU | 878 | 29.359 | 11.646 | 40.871 | 1.00 | 17.19 |
| ATOM | 602 | N | HIS | 879 | 30.174 | 10.245 | 42.429 | 1.00 | 14.53 |
| ATOM | 603 | H | HIS | 879 | 30.064 | 9.366 | 42.848 | 1.00 | 0.00 |
| ATOM | 604 | CA | HIS | 879 | 31.164 | 11.179 | 42.972 | 1.00 | 13.18 |
| ATOM | 605 | C | HIS | 879 | 32.196 | 11.499 | 41.855 | 1.00 | 14.44 |
| ATOM | 606 | O | HIS | 879 | 32.599 | 12.641 | 41.618 | 1.00 | 13.48 |
| ATOM | 607 | CB | HIS | 879 | 31.871 | 10.499 | 44.140 | 1.00 | 13.05 |
| ATOM | 608 | CG | HIS | 879 | 32.877 | 11.371 | 44.777 | 1.00 | 12.16 |
| ATOM | 609 | ND1 | HIS | 879 | 34.128 | 11.656 | 44.313 | 1.00 | 15.87 |
| ATOM | 610 | HD1 | HIS | 879 | 34.558 | 11.291 | 43.520 | 1.00 | 0.00 |
| ATOM | 611 | CD2 | HIS | 879 | 32.682 | 12.073 | 45.963 | 1.00 | 10.17 |
| ATOM | 612 | NE2 | HIS | 879 | 33.773 | 12.817 | 46.219 | 1.00 | 12.32 |
| ATOM | 613 | CE1 | HIS | 879 | 34.693 | 12.549 | 45.202 | 1.00 | 13.14 |
| ATOM | 614 | N | ARG | 880 | 32.671 | 10.477 | 41.138 | 1.00 | 13.49 |
| ATOM | 615 | H | ARG | 880 | 32.463 | 9.563 | 41.432 | 1.00 | 0.00 |
| ATOM | 616 | CA | ARG | 880 | 33.617 | 10.730 | 40.059 | 1.00 | 14.62 |
| ATOM | 617 | CB | ARG | 880 | 34.102 | 9.412 | 39.401 | 1.00 | 15.06 |
| ATOM | 618 | CG | ARG | 880 | 34.787 | 8.445 | 40.317 | 1.00 | 16.69 |
| ATOM | 619 | CD | ARG | 880 | 35.519 | 7.328 | 39.467 | 1.00 | 23.68 |
| ATOM | 620 | NE | ARG | 880 | 34.736 | 6.086 | 39.571 | 1.00 | 29.10 |
| ATOM | 621 | HE | ARG | 880 | 33.876 | 6.073 | 39.105 | 1.00 | 0.00 |
| ATOM | 622 | CZ | ARG | 880 | 35.042 | 4.952 | 40.234 | 1.00 | 27.39 |
| ATOM | 623 | NH1 | ARG | 880 | 34.126 | 4.012 | 40.168 | 1.00 | 30.27 |
| ATOM | 624 | HH11 | ARG | 880 | 33.296 | 4.168 | 39.639 | 1.00 | 0.00 |
| ATOM | 625 | HH12 | ARG | 880 | 34.254 | 3.141 | 40.637 | 1.00 | 0.00 |
| ATOM | 626 | NH2 | ARG | 880 | 36.151 | 4.685 | 40.914 | 1.00 | 27.03 |
| ATOM | 627 | HH21 | ARG | 880 | 36.888 | 5.361 | 40.974 | 1.00 | 0.00 |
| ATOM | 628 | HH22 | ARG | 880 | 36.237 | 3.797 | 41.366 | 1.00 | 0.00 |
| ATOM | 629 | C | ARG | 880 | 32.932 | 11.616 | 38.967 | 1.00 | 15.19 |
| ATOM | 630 | O | ARG | 880 | 33.520 | 12.562 | 38.392 | 1.00 | 13.81 |
| ATOM | 631 | N | ALA | 881 | 31.662 | 11.290 | 38.640 | 1.00 | 13.75 |
| ATOM | 632 | H | ALA | 881 | 31.242 | 10.548 | 39.121 | 1.00 | 0.00 |
| ATOM | 633 | CA | ALA | 881 | 30.948 | 12.061 | 37.611 | 1.00 | 13.97 |
| ATOM | 634 | CB | ALA | 881 | 29.605 | 11.450 | 37.421 | 1.00 | 10.50 |
| ATOM | 635 | C | ALA | 881 | 30.808 | 13.544 | 38.033 | 1.00 | 15.00 |
| ATOM | 636 | O | ALA | 881 | 30.962 | 14.464 | 37.223 | 1.00 | 14.61 |
| ATOM | 637 | N | LEU | 882 | 30.582 | 13.803 | 39.329 | 1.00 | 12.05 |
| ATOM | 638 | H | LEU | 882 | 30.406 | 13.042 | 39.924 | 1.00 | 0.00 |
| ATOM | 639 | CA | LEU | 882 | 30.486 | 15.188 | 39.878 | 1.00 | 12.56 |
| ATOM | 640 | CB | LEU | 882 | 30.012 | 15.207 | 41.358 | 1.00 | 10.34 |
| ATOM | 641 | CG | LEU | 882 | 29.649 | 16.587 | 41.955 | 1.00 | 8.74 |
| ATOM | 642 | CD1 | LEU | 882 | 28.687 | 17.382 | 41.035 | 1.00 | 6.47 |
| ATOM | 643 | CD2 | LEU | 882 | 28.922 | 16.335 | 43.283 | 1.00 | 7.41 |
| ATOM | 644 | C | LEU | 882 | 31.810 | 15.906 | 39.830 | 1.00 | 12.49 |
| ATOM | 645 | O | LEU | 882 | 31.893 | 17.089 | 39.517 | 1.00 | 15.28 |
| ATOM | 646 | N | MET | 883 | 32.868 | 15.214 | 40.200 | 1.00 | 13.01 |
| ATOM | 647 | H | MET | 883 | 32.749 | 14.299 | 40.528 | 1.00 | 0.00 |
| ATOM | 648 | CA | MET | 883 | 34.212 | 15.806 | 40.153 | 1.00 | 15.06 |
| ATOM | 649 | CB | MET | 883 | 35.256 | 14.813 | 40.749 | 1.00 | 17.54 |
| ATOM | 650 | CG | MET | 883 | 36.684 | 15.413 | 40.741 | 1.00 | 21.25 |
| ATOM | 651 | SD | MET | 883 | 36.907 | 16.883 | 41.840 | 1.00 | 22.22 |
| ATOM | 652 | CE | MET | 883 | 36.743 | 16.154 | 43.456 | 1.00 | 23.63 |
| ATOM | 653 | C | MET | 883 | 34.557 | 16.103 | 38.688 | 1.00 | 15.95 |
| ATOM | 654 | O | MET | 883 | 35.149 | 17.153 | 38.372 | 1.00 | 16.92 |
| ATOM | 655 | N | SER | 884 | 34.258 | 15.214 | 37.708 | 1.00 | 14.57 |
| ATOM | 656 | H | SER | 884 | 33.789 | 14.372 | 37.897 | 1.00 | 0.00 |
| ATOM | 657 | CA | SER | 884 | 34.640 | 15.650 | 36.317 | 1.00 | 14.10 |
| ATOM | 658 | CB | SER | 884 | 34.667 | 14.334 | 35.444 | 1.00 | 13.31 |
| ATOM | 659 | OG | SER | 884 | 33.337 | 14.011 | 35.258 | 1.00 | 24.99 |
| ATOM | 660 | HG | SER | 884 | 33.237 | 13.209 | 34.729 | 1.00 | 0.00 |
| ATOM | 661 | C | SER | 884 | 33.725 | 16.821 | 35.802 | 1.00 | 13.45 |
| ATOM | 662 | O | SER | 884 | 34.103 | 17.616 | 34.931 | 1.00 | 14.24 |
| ATOM | 663 | N | GLU | 885 | 32.520 | 16.987 | 36.378 | 1.00 | 12.27 |
| ATOM | 664 | H | GLU | 885 | 32.188 | 16.272 | 36.959 | 1.00 | 0.00 |
| ATOM | 665 | CA | GLU | 885 | 31.607 | 18.101 | 36.072 | 1.00 | 10.72 |
| ATOM | 666 | CB | GLU | 885 | 30.325 | 17.926 | 36.817 | 1.00 | 9.82 |
| ATOM | 667 | CG | GLU | 885 | 29.469 | 19.182 | 36.639 | 1.00 | 14.09 |
| ATOM | 668 | CD | GLU | 885 | 28.037 | 18.923 | 37.098 | 1.00 | 19.03 |
| ATOM | 669 | OE1 | GLU | 885 | 27.542 | 17.782 | 36.980 | 1.00 | 26.53 |
| ATOM | 670 | OE2 | GLU | 885 | 27.420 | 19.880 | 37.566 | 1.00 | 16.61 |
| ATOM | 671 | C | GLU | 885 | 32.267 | 19.409 | 36.510 | 1.00 | 12.62 |
| ATOM | 672 | O | GLU | 885 | 32.274 | 20.430 | 35.807 | 1.00 | 12.33 |
| ATOM | 673 | N | LEU | 886 | 32.838 | 19.356 | 37.716 | 1.00 | 13.13 |
| ATOM | 674 | H | LEU | 886 | 32.720 | 18.533 | 38.241 | 1.00 | 0.00 |
| ATOM | 675 | CA | LEU | 886 | 33.579 | 20.497 | 38.298 | 1.00 | 13.02 |
| ATOM | 676 | CB | LEU | 886 | 34.043 | 20.122 | 39.742 | 1.00 | 12.35 |
| ATOM | 677 | CG | LEU | 886 | 35.068 | 21.088 | 40.446 | 1.00 | 14.26 |
| ATOM | 678 | CD1 | LEU | 886 | 34.462 | 22.506 | 40.605 | 1.00 | 9.80 |
| ATOM | 679 | CD2 | LEU | 886 | 35.454 | 20.484 | 41.837 | 1.00 | 11.87 |
| ATOM | 680 | C | LEU | 886 | 34.788 | 20.826 | 37.411 | 1.00 | 14.11 |
| ATOM | 681 | O | LEU | 886 | 35.055 | 21.983 | 37.085 | 1.00 | 11.75 |
| ATOM | 682 | N | LYS | 887 | 35.538 | 19.811 | 36.953 | 1.00 | 14.43 |
| ATOM | 683 | H | LYS | 887 | 35.326 | 18.905 | 37.265 | 1.00 | 0.00 |
| ATOM | 684 | CA | LYS | 887 | 36.705 | 20.036 | 36.077 | 1.00 | 14.57 |
| ATOM | 685 | CB | LYS | 887 | 37.468 | 18.694 | 35.864 | 1.00 | 11.81 |
| ATOM | 686 | CG | LYS | 887 | 38.322 | 18.439 | 37.116 | 1.00 | 15.19 |
| ATOM | 687 | CD | LYS | 887 | 38.871 | 17.038 | 36.949 | 1.00 | 17.96 |
| ATOM | 688 | CE | LYS | 887 | 39.720 | 16.584 | 38.141 | 1.00 | 20.67 |
| ATOM | 689 | NZ | LYS | 887 | 40.037 | 15.137 | 37.973 | 1.00 | 24.28 |
| ATOM | 690 | HZ1 | LYS | 887 | 40.332 | 14.939 | 36.995 | 1.00 | 0.00 |
| ATOM | 691 | HZ2 | LYS | 887 | 39.204 | 14.562 | 38.215 | 1.00 | 0.00 |
| ATOM | 692 | HZ3 | LYS | 887 | 40.817 | 14.895 | 38.618 | 1.00 | 0.00 |
| ATOM | 693 | C | LYS | 887 | 36.290 | 20.662 | 34.717 | 1.00 | 14.49 |
| ATOM | 694 | O | LYS | 887 | 36.953 | 21.562 | 34.184 | 1.00 | 13.71 |
| ATOM | 695 | N | ILE | 888 | 35.198 | 20.192 | 34.117 | 1.00 | 16.24 |
| ATOM | 696 | H | ILE | 888 | 34.741 | 19.431 | 34.528 | 1.00 | 0.00 |
| ATOM | 697 | CA | ILE | 888 | 34.729 | 20.749 | 32.834 | 1.00 | 17.03 |
| ATOM | 698 | CB | ILE | 888 | 33.638 | 19.771 | 32.225 | 1.00 | 19.65 |
| ATOM | 699 | CG2 | ILE | 888 | 32.193 | 20.016 | 32.654 | 1.00 | 21.23 |
| ATOM | 700 | CG1 | ILE | 888 | 33.611 | 20.074 | 30.716 | 1.00 | 24.76 |
| ATOM | 701 | CD1 | ILE | 888 | 34.928 | 19.712 | 29.981 | 1.00 | 31.10 |
| ATOM | 702 | C | ILE | 888 | 34.241 | 22.179 | 33.033 | 1.00 | 14.57 |
| ATOM | 703 | O | ILE | 888 | 34.505 | 23.021 | 32.215 | 1.00 | 16.65 |
| ATOM | 704 | N | LEU | 889 | 33.551 | 22.546 | 34.080 | 1.00 | 16.14 |
| ATOM | 705 | H | LEU | 889 | 33.300 | 21.840 | 34.706 | 1.00 | 0.00 |
| ATOM | 706 | CA | LEU | 889 | 33.121 | 23.940 | 34.344 | 1.00 | 14.74 |
| ATOM | 707 | CB | LEU | 889 | 32.275 | 24.025 | 35.652 | 1.00 | 16.30 |
| ATOM | 708 | CG | LEU | 889 | 30.869 | 23.367 | 35.535 | 1.00 | 14.14 |
| ATOM | 709 | CD1 | LEU | 889 | 30.131 | 23.327 | 36.891 | 1.00 | 9.58 |
| ATOM | 710 | CD2 | LEU | 889 | 30.040 | 24.238 | 34.530 | 1.00 | 15.80 |
| ATOM | 711 | C | LEU | 889 | 34.330 | 24.842 | 34.499 | 1.00 | 14.87 |
| ATOM | 712 | O | LEU | 889 | 34.306 | 25.994 | 34.106 | 1.00 | 13.66 |
| ATOM | 713 | N | ILE | 890 | 35.392 | 24.378 | 35.148 | 1.00 | 14.96 |
| ATOM | 714 | H | ILE | 890 | 35.317 | 23.503 | 35.599 | 1.00 | 0.00 |
| ATOM | 715 | CA | ILE | 890 | 36.652 | 25.146 | 35.235 | 1.00 | 15.19 |
| ATOM | 716 | CB | ILE | 890 | 37.727 | 24.414 | 36.053 | 1.00 | 13.74 |
| ATOM | 717 | CG2 | ILE | 890 | 39.091 | 25.195 | 35.951 | 1.00 | 10.82 |
| ATOM | 718 | CG1 | ILE | 890 | 37.172 | 24.230 | 37.526 | 1.00 | 13.53 |
| ATOM | 719 | CD1 | ILE | 890 | 38.110 | 23.521 | 38.496 | 1.00 | 12.02 |
| ATOM | 720 | C | ILE | 890 | 37.180 | 25.332 | 33.814 | 1.00 | 15.50 |
| ATOM | 721 | O | ILE | 890 | 37.554 | 26.436 | 33.439 | 1.00 | 19.10 |
| ATOM | 722 | N | HIS | 891 | 37.253 | 24.250 | 33.031 | 1.00 | 14.36 |
| ATOM | 723 | H | HIS | 891 | 37.060 | 23.376 | 33.435 | 1.00 | 0.00 |
| ATOM | 724 | CA | HIS | 891 | 37.728 | 24.302 | 31.630 | 1.00 | 15.07 |
| ATOM | 725 | C | HIS | 891 | 36.893 | 25.282 | 30.783 | 1.00 | 15.70 |
| ATOM | 726 | O | HIS | 891 | 37.398 | 26.138 | 30.080 | 1.00 | 15.56 |
| ATOM | 727 | CB | HIS | 891 | 37.656 | 22.895 | 31.039 | 1.00 | 15.22 |
| ATOM | 728 | CG | HIS | 891 | 37.849 | 23.014 | 29.588 | 1.00 | 20.02 |
| ATOM | 729 | ND1 | HIS | 891 | 39.069 | 23.260 | 29.028 | 1.00 | 20.56 |
| ATOM | 730 | CE1 | HIS | 891 | 38.817 | 23.458 | 27.705 | 1.00 | 21.29 |
| ATOM | 731 | CD2 | HIS | 891 | 36.845 | 23.055 | 28.607 | 1.00 | 21.08 |
| ATOM | 732 | NE2 | HIS | 891 | 37.478 | 23.336 | 27.447 | 1.00 | 22.69 |
| ATOM | 733 | HE2 | HIS | 891 | 37.083 | 23.386 | 26.551 | 1.00 | 0.00 |
| ATOM | 734 | N | ILE | 892 | 35.586 | 25.165 | 30.814 | 1.00 | 13.74 |
| ATOM | 735 | H | ILE | 892 | 35.218 | 24.471 | 31.379 | 1.00 | 0.00 |
| ATOM | 736 | CA | ILE | 892 | 34.743 | 26.081 | 30.085 | 1.00 | 13.75 |
| ATOM | 737 | CB | ILE | 892 | 33.297 | 25.646 | 30.313 | 1.00 | 13.50 |
| ATOM | 738 | CG2 | ILE | 892 | 32.304 | 26.769 | 29.893 | 1.00 | 16.56 |
| ATOM | 739 | CG1 | ILE | 892 | 33.081 | 24.353 | 29.520 | 1.00 | 11.27 |
| ATOM | 740 | CD1 | ILE | 892 | 31.893 | 23.508 | 30.042 | 1.00 | 11.55 |
| ATOM | 741 | C | ILE | 892 | 34.991 | 27.531 | 30.540 | 1.00 | 14.09 |
| ATOM | 742 | O | ILE | 892 | 35.157 | 28.430 | 29.704 | 1.00 | 15.44 |
| ATOM | 743 | N | GLY | 893 | 35.084 | 27.835 | 31.813 | 1.00 | 13.03 |
| ATOM | 744 | H | GLY | 893 | 35.037 | 27.138 | 32.496 | 1.00 | 0.00 |
| ATOM | 745 | CA | GLY | 893 | 35.281 | 29.234 | 32.184 | 1.00 | 14.16 |
| ATOM | 746 | C | GLY | 893 | 33.985 | 30.127 | 32.052 | 1.00 | 14.83 |
| ATOM | 747 | O | GLY | 893 | 32.858 | 29.689 | 31.777 | 1.00 | 14.34 |
| ATOM | 748 | N | HIS | 894 | 34.081 | 31.440 | 32.266 | 1.00 | 13.94 |
| ATOM | 749 | H | HIS | 894 | 34.993 | 31.842 | 32.276 | 1.00 | 0.00 |
| ATOM | 750 | CA | HIS | 894 | 32.904 | 32.296 | 32.304 | 1.00 | 15.87 |
| ATOM | 751 | C | HIS | 894 | 32.339 | 32.942 | 31.046 | 1.00 | 14.94 |
| ATOM | 752 | O | HIS | 894 | 33.011 | 33.367 | 30.112 | 1.00 | 16.18 |
| ATOM | 753 | CB | HIS | 894 | 33.097 | 33.422 | 33.352 | 1.00 | 20.86 |
| ATOM | 754 | CG | HIS | 894 | 34.439 | 34.126 | 33.356 | 1.00 | 27.17 |
| ATOM | 755 | ND1 | HIS | 894 | 34.777 | 35.301 | 32.726 | 1.00 | 32.87 |
| ATOM | 756 | CE1 | HIS | 894 | 36.100 | 35.610 | 32.964 | 1.00 | 28.32 |
| ATOM | 757 | CD2 | HIS | 894 | 35.627 | 33.728 | 33.994 | 1.00 | 31.71 |
| ATOM | 758 | NE2 | HIS | 894 | 36.627 | 34.625 | 33.748 | 1.00 | 30.23 |
| ATOM | 759 | HE2 | HIS | 894 | 37.568 | 34.406 | 33.819 | 1.00 | 0.00 |
| ATOM | 760 | N | HIS | 895 | 31.009 | 33.020 | 31.066 | 1.00 | 12.95 |
| ATOM | 761 | H | HIS | 895 | 30.546 | 32.555 | 31.817 | 1.00 | 0.00 |
| ATOM | 762 | CA | HIS | 895 | 30.250 | 33.713 | 30.044 | 1.00 | 12.37 |
| ATOM | 763 | C | HIS | 895 | 28.971 | 34.050 | 30.773 | 1.00 | 11.82 |
| ATOM | 764 | O | HIS | 895 | 28.505 | 33.406 | 31.696 | 1.00 | 10.91 |
| ATOM | 765 | CB | HIS | 895 | 30.030 | 32.788 | 28.835 | 1.00 | 13.20 |
| ATOM | 766 | CG | HIS | 895 | 29.361 | 33.530 | 27.722 | 1.00 | 13.22 |
| ATOM | 767 | ND1 | HIS | 895 | 28.033 | 33.772 | 27.689 | 1.00 | 12.12 |
| ATOM | 768 | CE1 | HIS | 895 | 27.741 | 34.524 | 26.606 | 1.00 | 11.30 |
| ATOM | 769 | CD2 | HIS | 895 | 29.917 | 34.193 | 26.622 | 1.00 | 13.24 |
| ATOM | 770 | NE2 | HIS | 895 | 28.906 | 34.789 | 25.965 | 1.00 | 13.54 |
| ATOM | 771 | HE2 | HIS | 895 | 28.934 | 35.181 | 25.063 | 1.00 | 0.00 |
| ATOM | 772 | N | LEU | 896 | 28.440 | 35.180 | 30.412 | 1.00 | 12.35 |
| ATOM | 773 | H | LEU | 896 | 28.825 | 35.631 | 29.651 | 1.00 | 0.00 |
| ATOM | 774 | CA | LEU | 896 | 27.199 | 35.696 | 30.982 | 1.00 | 11.11 |
| ATOM | 775 | CB | LEU | 896 | 26.761 | 36.979 | 30.196 | 1.00 | 10.73 |
| ATOM | 776 | CG | LEU | 896 | 25.393 | 37.521 | 30.586 | 1.00 | 13.11 |
| ATOM | 777 | CD1 | LEU | 896 | 25.635 | 38.262 | 31.875 | 1.00 | 16.15 |
| ATOM | 778 | CD2 | LEU | 896 | 24.745 | 38.412 | 29.533 | 1.00 | 16.56 |
| ATOM | 779 | C | LEU | 896 | 26.102 | 34.652 | 30.849 | 1.00 | 12.16 |
| ATOM | 780 | O | LEU | 896 | 25.194 | 34.470 | 31.689 | 1.00 | 13.78 |
| ATOM | 781 | N | ASN | 897 | 26.151 | 33.933 | 29.717 | 1.00 | 14.10 |
| ATOM | 782 | H | ASN | 897 | 26.877 | 34.045 | 29.091 | 1.00 | 0.00 |
| ATOM | 783 | CA | ASN | 897 | 25.033 | 32.992 | 29.493 | 1.00 | 14.23 |
| ATOM | 784 | CB | ASN | 897 | 24.548 | 33.216 | 28.011 | 1.00 | 10.60 |
| ATOM | 785 | CG | ASN | 897 | 24.000 | 34.632 | 27.850 | 1.00 | 8.89 |
| ATOM | 786 | OD1 | ASN | 897 | 24.469 | 35.340 | 26.965 | 1.00 | 12.74 |
| ATOM | 787 | ND2 | ASN | 897 | 23.084 | 35.180 | 28.595 | 1.00 | 7.34 |
| ATOM | 788 | HD21 | ASN | 897 | 22.681 | 34.686 | 29.336 | 1.00 | 0.00 |
| ATOM | 789 | HD22 | ASN | 897 | 22.794 | 36.094 | 28.418 | 1.00 | 0.00 |
| ATOM | 790 | C | ASN | 897 | 25.198 | 31.532 | 29.864 | 1.00 | 14.34 |
| ATOM | 791 | O | ASN | 897 | 24.442 | 30.698 | 29.312 | 1.00 | 12.83 |
| ATOM | 792 | N | VAL | 898 | 26.181 | 31.206 | 30.689 | 1.00 | 13.20 |
| ATOM | 793 | H | VAL | 898 | 26.762 | 31.931 | 31.055 | 1.00 | 0.00 |
| ATOM | 794 | CA | VAL | 898 | 26.334 | 29.828 | 31.189 | 1.00 | 13.73 |
| ATOM | 795 | CB | VAL | 898 | 27.633 | 29.120 | 30.748 | 1.00 | 14.00 |
| ATOM | 796 | CG1 | VAL | 898 | 27.734 | 29.036 | 29.182 | 1.00 | 12.91 |
| ATOM | 797 | CG2 | VAL | 898 | 28.844 | 29.918 | 31.243 | 1.00 | 18.54 |
| ATOM | 798 | C | VAL | 898 | 26.367 | 30.029 | 32.702 | 1.00 | 16.15 |
| ATOM | 799 | O | VAL | 898 | 26.883 | 31.065 | 33.194 | 1.00 | 16.41 |
| ATOM | 800 | N | VAL | 899 | 25.710 | 29.203 | 33.524 | 1.00 | 16.66 |
| ATOM | 801 | H | VAL | 899 | 25.209 | 28.487 | 33.098 | 1.00 | 0.00 |
| ATOM | 802 | CA | VAL | 899 | 25.733 | 29.335 | 35.011 | 1.00 | 13.41 |
| ATOM | 803 | CB | VAL | 899 | 24.875 | 28.190 | 35.586 | 1.00 | 12.36 |
| ATOM | 804 | CG1 | VAL | 899 | 25.663 | 26.851 | 35.795 | 1.00 | 12.29 |
| ATOM | 805 | CG2 | VAL | 899 | 24.258 | 28.716 | 36.811 | 1.00 | 14.14 |
| ATOM | 806 | C | VAL | 899 | 27.257 | 29.266 | 35.325 | 1.00 | 14.63 |
| ATOM | 807 | O | VAL | 899 | 28.008 | 28.414 | 34.837 | 1.00 | 14.08 |
| ATOM | 808 | N | ASN | 900 | 27.753 | 30.257 | 36.077 | 1.00 | 12.74 |
| ATOM | 809 | H | ASN | 900 | 27.130 | 30.928 | 36.414 | 1.00 | 0.00 |
| ATOM | 810 | CA | ASN | 900 | 29.216 | 30.265 | 36.346 | 1.00 | 15.48 |
| ATOM | 811 | CB | ASN | 900 | 29.840 | 31.747 | 36.228 | 1.00 | 16.65 |
| ATOM | 812 | CG | ASN | 900 | 29.719 | 32.202 | 34.726 | 1.00 | 19.76 |
| ATOM | 813 | OD1 | ASN | 900 | 30.173 | 31.579 | 33.746 | 1.00 | 17.13 |
| ATOM | 814 | ND2 | ASN | 900 | 29.048 | 33.332 | 34.513 | 1.00 | 21.53 |
| ATOM | 815 | HD21 | ASN | 900 | 28.661 | 33.860 | 35.237 | 1.00 | 0.00 |
| ATOM | 816 | HD22 | ASN | 900 | 28.947 | 33.645 | 33.591 | 1.00 | 0.00 |
| ATOM | 817 | C | ASN | 900 | 29.601 | 29.664 | 37.654 | 1.00 | 12.21 |
| ATOM | 818 | O | ASN | 900 | 28.936 | 29.816 | 38.663 | 1.00 | 11.48 |
| ATOM | 819 | N | LEU | 901 | 30.678 | 28.933 | 37.585 | 1.00 | 14.90 |
| ATOM | 820 | H | LEU | 901 | 31.008 | 28.734 | 36.684 | 1.00 | 0.00 |
| ATOM | 821 | CA | LEU | 901 | 31.268 | 28.263 | 38.772 | 1.00 | 13.82 |
| ATOM | 822 | CB | LEU | 901 | 32.419 | 27.360 | 38.327 | 1.00 | 13.42 |
| ATOM | 823 | CG | LEU | 901 | 33.208 | 26.599 | 39.393 | 1.00 | 12.13 |
| ATOM | 824 | CD1 | LEU | 901 | 32.270 | 25.608 | 40.082 | 1.00 | 13.07 |
| ATOM | 825 | CD2 | LEU | 901 | 34.401 | 25.926 | 38.729 | 1.00 | 10.01 |
| ATOM | 826 | C | LEU | 901 | 31.814 | 29.320 | 39.716 | 1.00 | 13.75 |
| ATOM | 827 | O | LEU | 901 | 32.565 | 30.202 | 39.284 | 1.00 | 13.14 |
| ATOM | 828 | N | LEU | 902 | 31.453 | 29.247 | 40.954 | 1.00 | 13.47 |
| ATOM | 829 | H | LEU | 902 | 30.770 | 28.591 | 41.166 | 1.00 | 0.00 |
| ATOM | 830 | CA | LEU | 902 | 31.945 | 30.199 | 41.988 | 1.00 | 13.27 |
| ATOM | 831 | CB | LEU | 902 | 30.783 | 30.743 | 42.834 | 1.00 | 10.80 |
| ATOM | 832 | CG | LEU | 902 | 29.758 | 31.494 | 41.993 | 1.00 | 8.78 |
| ATOM | 833 | CD1 | LEU | 902 | 28.658 | 31.840 | 42.931 | 1.00 | 8.64 |
| ATOM | 834 | CD2 | LEU | 902 | 30.381 | 32.702 | 41.275 | 1.00 | 11.25 |
| ATOM | 835 | C | LEU | 902 | 32.946 | 29.525 | 42.913 | 1.00 | 14.73 |
| ATOM | 836 | O | LEU | 902 | 33.807 | 30.186 | 43.484 | 1.00 | 14.04 |
| ATOM | 837 | N | GLY | 903 | 32.890 | 28.215 | 43.061 | 1.00 | 14.83 |
| ATOM | 838 | H | GLY | 903 | 32.183 | 27.712 | 42.610 | 1.00 | 0.00 |
| ATOM | 839 | CA | GLY | 903 | 33.829 | 27.521 | 43.941 | 1.00 | 14.84 |
| ATOM | 840 | C | GLY | 903 | 33.430 | 26.067 | 44.142 | 1.00 | 14.86 |
| ATOM | 841 | O | GLY | 903 | 32.466 | 25.549 | 43.527 | 1.00 | 15.21 |
| ATOM | 842 | N | ALA | 904 | 34.181 | 25.423 | 45.051 | 1.00 | 16.22 |
| ATOM | 843 | H | ALA | 904 | 34.944 | 25.898 | 45.458 | 1.00 | 0.00 |
| ATOM | 844 | CA | ALA | 904 | 33.943 | 24.000 | 45.387 | 1.00 | 14.64 |
| ATOM | 845 | CB | ALA | 904 | 34.593 | 23.023 | 44.357 | 1.00 | 14.27 |
| ATOM | 846 | C | ALA | 904 | 34.498 | 23.569 | 46.715 | 1.00 | 13.72 |
| ATOM | 847 | O | ALA | 904 | 35.480 | 24.102 | 47.201 | 1.00 | 13.85 |
| ATOM | 848 | N | CYS | 905 | 33.915 | 22.549 | 47.279 | 1.00 | 13.63 |
| ATOM | 849 | H | CYS | 905 | 33.141 | 22.159 | 46.812 | 1.00 | 0.00 |
| ATOM | 850 | CA | CYS | 905 | 34.385 | 21.978 | 48.550 | 1.00 | 13.62 |
| ATOM | 851 | CB | CYS | 905 | 33.332 | 21.887 | 49.585 | 1.00 | 10.95 |
| ATOM | 852 | SG | CYS | 905 | 32.487 | 23.476 | 49.837 | 1.00 | 17.33 |
| ATOM | 853 | C | CYS | 905 | 34.726 | 20.562 | 48.182 | 1.00 | 12.34 |
| ATOM | 854 | O | CYS | 905 | 33.866 | 19.776 | 47.836 | 1.00 | 14.23 |
| ATOM | 855 | N | THR | 906 | 36.005 | 20.281 | 48.010 | 1.00 | 14.19 |
| ATOM | 856 | H | THR | 906 | 36.635 | 21.021 | 48.047 | 1.00 | 0.00 |
| ATOM | 857 | CA | THR | 906 | 36.522 | 18.915 | 47.653 | 1.00 | 16.23 |
| ATOM | 858 | CB | THR | 906 | 37.374 | 19.009 | 46.365 | 1.00 | 14.52 |
| ATOM | 859 | OG1 | THR | 906 | 38.530 | 19.728 | 46.736 | 1.00 | 14.01 |
| ATOM | 860 | HG1 | THR | 906 | 39.238 | 19.626 | 46.092 | 1.00 | 0.00 |
| ATOM | 861 | CG2 | THR | 906 | 36.668 | 19.719 | 45.170 | 1.00 | 13.52 |
| ATOM | 862 | C | THR | 906 | 37.382 | 18.235 | 48.787 | 1.00 | 19.87 |
| ATOM | 863 | O | THR | 906 | 37.774 | 17.063 | 48.724 | 1.00 | 19.63 |
| ATOM | 864 | N | LYS | 907 | 37.669 | 18.962 | 49.900 | 1.00 | 22.16 |
| ATOM | 865 | H | LYS | 907 | 37.280 | 19.853 | 49.972 | 1.00 | 0.00 |
| ATOM | 866 | CA | LYS | 907 | 38.484 | 18.434 | 50.996 | 1.00 | 22.66 |
| ATOM | 867 | CB | LYS | 907 | 38.564 | 19.475 | 52.174 | 1.00 | 20.32 |
| ATOM | 868 | C | LYS | 907 | 37.885 | 17.120 | 51.504 | 1.00 | 23.58 |
| ATOM | 869 | O | LYS | 907 | 36.677 | 16.976 | 51.611 | 1.00 | 19.77 |
| ATOM | 870 | N | PRO | 908 | 38.743 | 16.123 | 51.785 | 1.00 | 25.80 |
| ATOM | 871 | CD | PRO | 908 | 40.095 | 15.964 | 51.288 | 1.00 | 24.87 |
| ATOM | 872 | CA | PRO | 908 | 38.321 | 14.836 | 52.355 | 1.00 | 26.87 |
| ATOM | 873 | CB | PRO | 908 | 39.629 | 14.064 | 52.639 | 1.00 | 26.07 |
| ATOM | 874 | CG | PRO | 908 | 40.658 | 15.137 | 52.441 | 1.00 | 27.75 |
| ATOM | 875 | C | PRO | 908 | 37.403 | 14.854 | 53.540 | 1.00 | 26.34 |
| ATOM | 876 | O | PRO | 908 | 36.615 | 13.926 | 53.704 | 1.00 | 28.47 |
| ATOM | 877 | N | GLY | 909 | 37.377 | 15.825 | 54.434 | 1.00 | 26.65 |
| ATOM | 878 | H | GLY | 909 | 37.969 | 16.599 | 54.372 | 1.00 | 0.00 |
| ATOM | 879 | CA | GLY | 909 | 36.349 | 15.640 | 55.559 | 1.00 | 30.09 |
| ATOM | 880 | C | GLY | 909 | 34.796 | 15.370 | 55.324 | 1.00 | 28.00 |
| ATOM | 881 | O | GLY | 909 | 34.047 | 14.826 | 56.160 | 1.00 | 25.68 |
| ATOM | 882 | N | GLY | 910 | 34.295 | 15.855 | 54.169 | 1.00 | 25.82 |
| ATOM | 883 | H | GLY | 910 | 34.930 | 16.204 | 53.506 | 1.00 | 0.00 |
| ATOM | 884 | CA | GLY | 910 | 32.877 | 15.790 | 53.822 | 1.00 | 21.05 |
| ATOM | 885 | C | GLY | 910 | 32.627 | 15.660 | 52.320 | 1.00 | 18.76 |
| ATOM | 886 | O | GLY | 910 | 33.501 | 15.226 | 51.546 | 1.00 | 16.41 |
| ATOM | 887 | N | PRO | 911 | 31.359 | 15.944 | 51.951 | 1.00 | 18.47 |
| ATOM | 888 | CD | PRO | 911 | 30.308 | 16.529 | 52.802 | 1.00 | 18.27 |
| ATOM | 889 | CA | PRO | 911 | 30.860 | 15.663 | 50.584 | 1.00 | 17.24 |
| ATOM | 890 | CB | PRO | 911 | 29.375 | 15.665 | 50.719 | 1.00 | 17.52 |
| ATOM | 891 | CG | PRO | 911 | 29.112 | 16.652 | 51.836 | 1.00 | 16.70 |
| ATOM | 892 | C | PRO | 911 | 31.319 | 16.618 | 49.494 | 1.00 | 16.65 |
| ATOM | 893 | O | PRO | 911 | 31.622 | 17.781 | 49.771 | 1.00 | 13.36 |
| ATOM | 894 | N | LEU | 912 | 31.525 | 16.111 | 48.264 | 1.00 | 15.21 |
| ATOM | 895 | H | LEU | 912 | 31.408 | 15.149 | 48.124 | 1.00 | 0.00 |
| ATOM | 896 | CA | LEU | 912 | 31.944 | 17.021 | 47.158 | 1.00 | 15.31 |
| ATOM | 897 | CB | LEU | 912 | 32.296 | 16.164 | 45.930 | 1.00 | 14.16 |
| ATOM | 898 | CG | LEU | 912 | 32.612 | 16.895 | 44.600 | 1.00 | 15.67 |
| ATOM | 899 | CD1 | LEU | 912 | 33.761 | 17.914 | 44.703 | 1.00 | 15.72 |
| ATOM | 900 | CD2 | LEU | 912 | 32.946 | 15.762 | 43.574 | 1.00 | 13.95 |
| ATOM | 901 | C | LEU | 912 | 30.775 | 18.005 | 46.868 | 1.00 | 13.39 |
| ATOM | 902 | O | LEU | 912 | 29.591 | 17.626 | 46.803 | 1.00 | 12.84 |
| ATOM | 903 | N | MET | 913 | 31.096 | 19.273 | 46.724 | 1.00 | 12.45 |
| ATOM | 904 | H | MET | 913 | 32.039 | 19.519 | 46.851 | 1.00 | 0.00 |
| ATOM | 905 | CA | MET | 913 | 30.084 | 20.290 | 46.470 | 1.00 | 13.51 |
| ATOM | 906 | CB | MET | 913 | 29.846 | 21.164 | 47.714 | 1.00 | 13.67 |
| ATOM | 907 | CG | MET | 913 | 29.403 | 20.259 | 48.869 | 1.00 | 20.74 |
| ATOM | 908 | SD | MET | 913 | 28.999 | 21.300 | 50.278 | 1.00 | 27.33 |
| ATOM | 909 | CE | MET | 913 | 27.525 | 20.473 | 50.704 | 1.00 | 22.61 |
| ATOM | 910 | C | MET | 913 | 30.614 | 21.181 | 45.376 | 1.00 | 12.84 |
| ATOM | 911 | O | MET | 913 | 31.780 | 21.597 | 45.435 | 1.00 | 12.13 |
| ATOM | 912 | N | VAL | 914 | 29.739 | 21.466 | 44.387 | 1.00 | 12.96 |
| ATOM | 913 | H | VAL | 914 | 28.860 | 21.030 | 44.437 | 1.00 | 0.00 |
| ATOM | 914 | CA | VAL | 914 | 30.070 | 22.320 | 43.229 | 1.00 | 9.11 |
| ATOM | 915 | CB | VAL | 914 | 29.902 | 21.508 | 41.889 | 1.00 | 10.67 |
| ATOM | 916 | CG1 | VAL | 914 | 30.305 | 22.396 | 40.671 | 1.00 | 8.37 |
| ATOM | 917 | CG2 | VAL | 914 | 30.806 | 20.216 | 41.964 | 1.00 | 5.03 |
| ATOM | 918 | C | VAL | 914 | 29.092 | 23.469 | 43.326 | 1.00 | 9.81 |
| ATOM | 919 | O | VAL | 914 | 27.879 | 23.286 | 43.263 | 1.00 | 9.77 |
| ATOM | 920 | N | ILE | 915 | 29.688 | 24.648 | 43.542 | 1.00 | 10.77 |
| ATOM | 921 | H | ILE | 915 | 30.669 | 24.660 | 43.541 | 1.00 | 0.00 |
| ATOM | 922 | CA | ILE | 915 | 28.998 | 25.918 | 43.755 | 1.00 | 10.86 |
| ATOM | 923 | CB | ILE | 915 | 29.661 | 26.682 | 44.962 | 1.00 | 10.11 |
| ATOM | 924 | CG2 | ILE | 915 | 28.769 | 27.895 | 45.383 | 1.00 | 8.97 |
| ATOM | 925 | CG1 | ILE | 915 | 29.840 | 25.698 | 46.156 | 1.00 | 11.93 |
| ATOM | 926 | CD1 | ILE | 915 | 30.902 | 26.189 | 47.135 | 1.00 | 13.57 |
| ATOM | 927 | C | ILE | 915 | 28.961 | 26.836 | 42.538 | 1.00 | 11.04 |
| ATOM | 928 | O | ILE | 915 | 29.994 | 27.216 | 42.015 | 1.00 | 11.73 |
| ATOM | 929 | N | VAL | 916 | 27.769 | 27.170 | 42.080 | 1.00 | 11.04 |
| ATOM | 930 | H | VAL | 916 | 26.979 | 26.803 | 42.534 | 1.00 | 0.00 |
| ATOM | 931 | CA | VAL | 916 | 27.595 | 28.079 | 40.919 | 1.00 | 11.96 |
| ATOM | 932 | CB | VAL | 916 | 27.044 | 27.322 | 39.611 | 1.00 | 10.66 |
| ATOM | 933 | CG1 | VAL | 916 | 28.025 | 26.246 | 39.105 | 1.00 | 9.22 |
| ATOM | 934 | CG2 | VAL | 916 | 25.647 | 26.715 | 39.948 | 1.00 | 7.76 |
| ATOM | 935 | C | VAL | 916 | 26.591 | 29.203 | 41.266 | 1.00 | 12.00 |
| ATOM | 936 | O | VAL | 916 | 25.937 | 29.255 | 42.334 | 1.00 | 12.56 |
| ATOM | 937 | N | GLU | 917 | 26.488 | 30.182 | 40.363 | 1.00 | 13.08 |
| ATOM | 938 | H | GLU | 917 | 27.070 | 30.116 | 39.576 | 1.00 | 0.00 |
| ATOM | 939 | CA | GLU | 917 | 25.529 | 31.308 | 40.542 | 1.00 | 13.66 |
| ATOM | 940 | CB | GLU | 917 | 25.358 | 32.166 | 39.236 | 1.00 | 16.76 |
| ATOM | 941 | CG | GLU | 917 | 26.769 | 32.642 | 38.993 | 1.00 | 20.20 |
| ATOM | 942 | CD | GLU | 917 | 26.873 | 33.418 | 37.686 | 1.00 | 27.74 |
| ATOM | 943 | OE1 | GLU | 917 | 27.211 | 34.610 | 37.787 | 1.00 | 31.60 |
| ATOM | 944 | OE2 | GLU | 917 | 26.620 | 32.837 | 36.615 | 1.00 | 23.87 |
| ATOM | 945 | C | GLU | 917 | 24.113 | 30.837 | 40.839 | 1.00 | 14.51 |
| ATOM | 946 | O | GLU | 917 | 23.673 | 29.851 | 40.223 | 1.00 | 15.96 |
| ATOM | 947 | N | PHE | 918 | 23.372 | 31.568 | 41.685 | 1.00 | 13.12 |
| ATOM | 948 | H | PHE | 918 | 23.809 | 32.342 | 42.093 | 1.00 | 0.00 |
| ATOM | 949 | CA | PHE | 918 | 21.968 | 31.300 | 41.966 | 1.00 | 11.78 |
| ATOM | 950 | CB | PHE | 918 | 21.605 | 31.783 | 43.351 | 1.00 | 13.55 |
| ATOM | 951 | CG | PHE | 918 | 20.174 | 31.619 | 43.632 | 1.00 | 14.06 |
| ATOM | 952 | CD1 | PHE | 918 | 19.593 | 30.360 | 43.688 | 1.00 | 15.10 |
| ATOM | 953 | CD2 | PHE | 918 | 19.393 | 32.744 | 43.856 | 1.00 | 15.48 |
| ATOM | 954 | CE1 | PHE | 918 | 18.229 | 30.236 | 43.982 | 1.00 | 19.00 |
| ATOM | 955 | CE2 | PHE | 918 | 18.027 | 32.597 | 44.154 | 1.00 | 17.39 |
| ATOM | 956 | CZ | PHE | 918 | 17.413 | 31.347 | 44.225 | 1.00 | 17.31 |
| ATOM | 957 | C | PHE | 918 | 21.208 | 32.172 | 40.910 | 1.00 | 13.10 |
| ATOM | 958 | O | PHE | 918 | 21.465 | 33.365 | 40.703 | 1.00 | 13.32 |
| ATOM | 959 | N | CYS | 919 | 20.208 | 31.611 | 40.266 | 1.00 | 13.28 |
| ATOM | 960 | H | CYS | 919 | 20.046 | 30.682 | 40.504 | 1.00 | 0.00 |
| ATOM | 961 | CA | CYS | 919 | 19.336 | 32.256 | 39.219 | 1.00 | 13.58 |
| ATOM | 962 | CB | CYS | 919 | 19.345 | 31.418 | 37.918 | 1.00 | 9.79 |
| ATOM | 963 | SG | CYS | 919 | 20.995 | 31.366 | 37.196 | 1.00 | 14.47 |
| ATOM | 964 | C | CYS | 919 | 17.914 | 32.297 | 39.788 | 1.00 | 12.95 |
| ATOM | 965 | O | CYS | 919 | 17.188 | 31.309 | 39.799 | 1.00 | 13.90 |
| ATOM | 966 | N | LYS | 920 | 17.561 | 33.468 | 40.282 | 1.00 | 14.69 |
| ATOM | 967 | H | LYS | 920 | 18.250 | 34.146 | 40.212 | 1.00 | 0.00 |
| ATOM | 968 | CA | LYS | 920 | 16.290 | 33.877 | 40.908 | 1.00 | 17.94 |
| ATOM | 969 | CB | LYS | 920 | 16.060 | 35.426 | 40.610 | 1.00 | 20.97 |
| ATOM | 970 | CG | LYS | 920 | 15.875 | 36.389 | 41.751 | 1.00 | 27.53 |
| ATOM | 971 | CD | LYS | 920 | 17.087 | 35.968 | 42.656 | 1.00 | 37.07 |
| ATOM | 972 | CE | LYS | 920 | 17.986 | 37.040 | 43.399 | 1.00 | 38.58 |
| ATOM | 973 | NZ | LYS | 920 | 19.299 | 36.462 | 43.736 | 1.00 | 36.98 |
| ATOM | 974 | HZ1 | LYS | 920 | 19.735 | 36.119 | 42.860 | 1.00 | 0.00 |
| ATOM | 975 | HZ2 | LYS | 920 | 19.175 | 35.684 | 44.414 | 1.00 | 0.00 |
| ATOM | 976 | HZ3 | LYS | 920 | 19.882 | 37.208 | 44.166 | 1.00 | 0.00 |
| ATOM | 977 | C | LYS | 920 | 15.006 | 33.132 | 40.479 | 1.00 | 17.07 |
| ATOM | 978 | O | LYS | 920 | 14.234 | 32.608 | 41.274 | 1.00 | 15.03 |
| ATOM | 979 | N | PHE | 921 | 14.740 | 33.124 | 39.192 | 1.00 | 16.05 |
| ATOM | 980 | H | PHE | 921 | 15.389 | 33.491 | 38.565 | 1.00 | 0.00 |
| ATOM | 981 | CA | PHE | 921 | 13.517 | 32.565 | 38.649 | 1.00 | 14.85 |
| ATOM | 982 | CB | PHE | 921 | 13.202 | 33.470 | 37.430 | 1.00 | 16.68 |
| ATOM | 983 | CG | PHE | 921 | 12.916 | 34.845 | 37.872 | 1.00 | 16.40 |
| ATOM | 984 | CD1 | PHE | 921 | 11.672 | 35.158 | 38.477 | 1.00 | 23.57 |
| ATOM | 985 | CD2 | PHE | 921 | 13.873 | 35.844 | 37.680 | 1.00 | 16.60 |
| ATOM | 986 | CE1 | PHE | 921 | 11.399 | 36.487 | 38.882 | 1.00 | 23.79 |
| ATOM | 987 | CE2 | PHE | 921 | 13.636 | 37.169 | 38.070 | 1.00 | 21.22 |
| ATOM | 988 | CZ | PHE | 921 | 12.397 | 37.480 | 38.671 | 1.00 | 25.48 |
| ATOM | 989 | C | PHE | 921 | 13.443 | 31.123 | 38.319 | 1.00 | 13.49 |
| ATOM | 990 | O | PHE | 921 | 12.400 | 30.616 | 37.910 | 1.00 | 15.83 |
| ATOM | 991 | N | GLY | 922 | 14.554 | 30.440 | 38.421 | 1.00 | 12.47 |
| ATOM | 992 | H | GLY | 922 | 15.375 | 30.895 | 38.701 | 1.00 | 0.00 |
| ATOM | 993 | CA | GLY | 922 | 14.523 | 29.006 | 38.091 | 1.00 | 13.53 |
| ATOM | 994 | C | GLY | 922 | 14.573 | 28.677 | 36.589 | 1.00 | 13.20 |
| ATOM | 995 | O | GLY | 922 | 14.912 | 29.531 | 35.744 | 1.00 | 14.40 |
| ATOM | 996 | N | ASN | 923 | 14.200 | 27.438 | 36.253 | 1.00 | 12.52 |
| ATOM | 997 | H | ASN | 923 | 13.898 | 26.838 | 36.970 | 1.00 | 0.00 |
| ATOM | 998 | CA | ASN | 923 | 14.235 | 27.033 | 34.863 | 1.00 | 12.68 |
| ATOM | 999 | CB | ASN | 923 | 14.150 | 25.500 | 34.849 | 1.00 | 14.05 |
| ATOM | 1000 | CG | ASN | 923 | 12.744 | 24.948 | 34.771 | 1.00 | 17.84 |
| ATOM | 1001 | OD1 | ASN | 923 | 12.136 | 24.813 | 33.704 | 1.00 | 19.20 |
| ATOM | 1002 | ND2 | ASN | 923 | 12.106 | 24.628 | 35.881 | 1.00 | 19.07 |
| ATOM | 1003 | HD21 | ASN | 923 | 12.526 | 24.769 | 36.762 | 1.00 | 0.00 |
| ATOM | 1004 | HD22 | ASN | 923 | 11.206 | 24.266 | 35.784 | 1.00 | 0.00 |
| ATOM | 1005 | C | ASN | 923 | 13.173 | 27.736 | 34.007 | 1.00 | 12.41 |
| ATOM | 1006 | O | ASN | 923 | 12.018 | 28.007 | 34.382 | 1.00 | 13.67 |
| ATOM | 1007 | N | LEU | 924 | 13.594 | 27.890 | 32.765 | 1.00 | 13.02 |
| ATOM | 1008 | H | LEU | 924 | 14.445 | 27.484 | 32.517 | 1.00 | 0.00 |
| ATOM | 1009 | CA | LEU | 924 | 12.839 | 28.607 | 31.772 | 1.00 | 12.69 |
| ATOM | 1010 | CB | LEU | 924 | 13.769 | 28.864 | 30.556 | 1.00 | 12.38 |
| ATOM | 1011 | CG | LEU | 924 | 13.226 | 29.914 | 29.560 | 1.00 | 11.67 |
| ATOM | 1012 | CD1 | LEU | 924 | 13.101 | 31.297 | 30.252 | 1.00 | 12.30 |
| ATOM | 1013 | CD2 | LEU | 924 | 14.141 | 29.906 | 28.328 | 1.00 | 12.81 |
| ATOM | 1014 | C | LEU | 924 | 11.584 | 27.926 | 31.359 | 1.00 | 13.25 |
| ATOM | 1015 | O | LEU | 924 | 10.596 | 28.623 | 31.115 | 1.00 | 12.81 |
| ATOM | 1016 | N | SER | 925 | 11.568 | 26.601 | 31.309 | 1.00 | 12.12 |
| ATOM | 1017 | H | SER | 925 | 12.389 | 26.119 | 31.534 | 1.00 | 0.00 |
| ATOM | 1018 | CA | SER | 925 | 10.331 | 25.905 | 30.901 | 1.00 | 15.80 |
| ATOM | 1019 | CB | SER | 925 | 10.671 | 24.414 | 30.833 | 1.00 | 13.89 |
| ATOM | 1020 | OG | SER | 925 | 9.435 | 23.742 | 30.576 | 1.00 | 21.06 |
| ATOM | 1021 | HG | SER | 925 | 9.522 | 22.790 | 30.468 | 1.00 | 0.00 |
| ATOM | 1022 | C | SER | 925 | 9.115 | 26.192 | 31.842 | 1.00 | 15.93 |
| ATOM | 1023 | O | SER | 925 | 8.029 | 26.668 | 31.489 | 1.00 | 14.03 |
| ATOM | 1024 | N | THR | 926 | 9.344 | 25.973 | 33.130 | 1.00 | 13.18 |
| ATOM | 1025 | H | THR | 926 | 10.222 | 25.589 | 33.335 | 1.00 | 0.00 |
| ATOM | 1026 | CA | THR | 926 | 8.371 | 26.216 | 34.195 | 1.00 | 13.87 |
| ATOM | 1027 | CB | THR | 926 | 9.009 | 25.768 | 35.525 | 1.00 | 14.33 |
| ATOM | 1028 | OG1 | THR | 926 | 9.249 | 24.349 | 35.481 | 1.00 | 14.49 |
| ATOM | 1029 | HG1 | THR | 926 | 9.473 | 24.050 | 36.373 | 1.00 | 0.00 |
| ATOM | 1030 | CG2 | THR | 926 | 8.104 | 26.110 | 36.730 | 1.00 | 17.44 |
| ATOM | 1031 | C | THR | 926 | 8.020 | 27.691 | 34.215 | 1.00 | 13.66 |
| ATOM | 1032 | O | THR | 926 | 6.892 | 28.148 | 34.365 | 1.00 | 13.96 |
| ATOM | 1033 | N | TYR | 927 | 9.038 | 28.540 | 34.127 | 1.00 | 14.23 |
| ATOM | 1034 | H | TYR | 927 | 9.959 | 28.217 | 34.008 | 1.00 | 0.00 |
| ATOM | 1035 | CA | TYR | 927 | 8.751 | 29.990 | 34.124 | 1.00 | 14.25 |
| ATOM | 1036 | CB | TYR | 927 | 10.109 | 30.802 | 34.112 | 1.00 | 12.67 |
| ATOM | 1037 | CG | TYR | 927 | 9.830 | 32.246 | 34.031 | 1.00 | 13.31 |
| ATOM | 1038 | CD1 | TYR | 927 | 9.425 | 32.931 | 35.185 | 1.00 | 16.73 |
| ATOM | 1039 | CE1 | TYR | 927 | 9.125 | 34.300 | 35.128 | 1.00 | 14.82 |
| ATOM | 1040 | CD2 | TYR | 927 | 9.940 | 32.935 | 32.793 | 1.00 | 17.12 |
| ATOM | 1041 | CE2 | TYR | 927 | 9.642 | 34.315 | 32.712 | 1.00 | 15.81 |
| ATOM | 1042 | CZ | TYR | 927 | 9.242 | 34.972 | 33.904 | 1.00 | 19.00 |
| ATOM | 1043 | OH | TYR | 927 | 9.021 | 36.335 | 33.937 | 1.00 | 21.47 |
| ATOM | 1044 | HH | TYR | 927 | 9.336 | 36.762 | 33.132 | 1.00 | 0.00 |
| ATOM | 1045 | C | TYR | 927 | 7.852 | 30.428 | 32.941 | 1.00 | 12.88 |
| ATOM | 1046 | O | TYR | 927 | 6.815 | 31.008 | 33.159 | 1.00 | 12.26 |
| ATOM | 1047 | N | LEU | 928 | 8.192 | 30.227 | 31.700 | 1.00 | 13.69 |
| ATOM | 1048 | H | LEU | 928 | 9.021 | 29.735 | 31.529 | 1.00 | 0.00 |
| ATOM | 1049 | CA | LEU | 928 | 7.345 | 30.634 | 30.556 | 1.00 | 13.88 |
| ATOM | 1050 | CB | LEU | 928 | 7.982 | 30.117 | 29.235 | 1.00 | 13.65 |
| ATOM | 1051 | CG | LEU | 928 | 9.317 | 30.796 | 28.927 | 1.00 | 11.45 |
| ATOM | 1052 | CD1 | LEU | 928 | 9.861 | 30.129 | 27.650 | 1.00 | 13.54 |
| ATOM | 1053 | CD2 | LEU | 928 | 9.172 | 32.366 | 28.831 | 1.00 | 15.16 |
| ATOM | 1054 | C | LEU | 928 | 5.927 | 30.091 | 30.675 | 1.00 | 15.55 |
| ATOM | 1055 | O | LEU | 928 | 4.961 | 30.767 | 30.331 | 1.00 | 16.93 |
| ATOM | 1056 | N | ARG | 929 | 5.783 | 28.856 | 31.172 | 1.00 | 16.97 |
| ATOM | 1057 | H | ARG | 929 | 6.594 | 28.337 | 31.364 | 1.00 | 0.00 |
| ATOM | 1058 | CA | ARG | 929 | 4.473 | 28.218 | 31.322 | 1.00 | 17.56 |
| ATOM | 1059 | CB | ARG | 929 | 4.752 | 26.808 | 31.693 | 1.00 | 19.04 |
| ATOM | 1060 | CG | ARG | 929 | 3.675 | 25.908 | 31.157 | 1.00 | 25.06 |
| ATOM | 1061 | CD | ARG | 929 | 4.304 | 24.529 | 31.219 | 1.00 | 35.73 |
| ATOM | 1062 | NE | ARG | 929 | 4.892 | 24.241 | 32.567 | 1.00 | 43.55 |
| ATOM | 1063 | HE | ARG | 929 | 4.432 | 24.582 | 33.363 | 1.00 | 0.00 |
| ATOM | 1064 | CZ | ARG | 929 | 6.042 | 23.567 | 32.757 | 1.00 | 43.64 |
| ATOM | 1065 | NH1 | ARG | 929 | 6.407 | 23.370 | 34.023 | 1.00 | 47.95 |
| ATOM | 1066 | HH11 | ARG | 929 | 5.855 | 23.713 | 34.783 | 1.00 | 0.00 |
| ATOM | 1067 | HH12 | ARG | 929 | 7.258 | 22.876 | 34.217 | 1.00 | 0.00 |
| ATOM | 1068 | NH2 | ARG | 929 | 6.845 | 23.153 | 31.753 | 1.00 | 42.44 |
| ATOM | 1069 | HH21 | ARG | 929 | 6.597 | 23.349 | 30.804 | 1.00 | 0.00 |
| ATOM | 1070 | HH22 | ARG | 929 | 7.688 | 22.652 | 31.955 | 1.00 | 0.00 |
| ATOM | 1071 | C | ARG | 929 | 3.617 | 28.948 | 32.343 | 1.00 | 19.75 |
| ATOM | 1072 | O | ARG | 929 | 2.399 | 28.845 | 32.352 | 1.00 | 22.13 |
| ATOM | 1073 | N | SER | 930 | 4.233 | 29.643 | 33.302 | 1.00 | 19.18 |
| ATOM | 1074 | H | SER | 930 | 5.210 | 29.597 | 33.337 | 1.00 | 0.00 |
| ATOM | 1075 | CA | SER | 930 | 3.516 | 30.450 | 34.297 | 1.00 | 17.14 |
| ATOM | 1076 | CB | SER | 930 | 4.368 | 30.611 | 35.573 | 1.00 | 17.28 |
| ATOM | 1077 | OG | SER | 930 | 5.396 | 31.558 | 35.356 | 1.00 | 15.44 |
| ATOM | 1078 | HG | SER | 930 | 5.998 | 31.536 | 36.103 | 1.00 | 0.00 |
| ATOM | 1079 | C | SER | 930 | 3.180 | 31.828 | 33.760 | 1.00 | 16.63 |
| ATOM | 1080 | O | SER | 930 | 2.441 | 32.546 | 34.424 | 1.00 | 16.64 |
| ATOM | 1081 | N | LYS | 931 | 3.678 | 32.262 | 32.579 | 1.00 | 16.58 |
| ATOM | 1082 | H | LYS | 931 | 4.294 | 31.661 | 32.120 | 1.00 | 0.00 |
| ATOM | 1083 | CA | LYS | 931 | 3.399 | 33.597 | 31.969 | 1.00 | 17.18 |
| ATOM | 1084 | CB | LYS | 931 | 4.715 | 34.194 | 31.452 | 1.00 | 16.65 |
| ATOM | 1085 | CG | LYS | 931 | 5.755 | 34.459 | 32.559 | 1.00 | 18.37 |
| ATOM | 1086 | CD | LYS | 931 | 5.072 | 34.948 | 33.845 | 1.00 | 20.53 |
| ATOM | 1087 | CE | LYS | 931 | 5.533 | 36.283 | 34.406 | 1.00 | 27.38 |
| ATOM | 1088 | NZ | LYS | 931 | 5.326 | 37.459 | 33.569 | 1.00 | 21.37 |
| ATOM | 1089 | HZ1 | LYS | 931 | 4.341 | 37.453 | 33.233 | 1.00 | 0.00 |
| ATOM | 1090 | HZ2 | LYS | 931 | 5.973 | 37.438 | 32.756 | 1.00 | 0.00 |
| ATOM | 1091 | HZ3 | LYS | 931 | 5.487 | 38.318 | 34.135 | 1.00 | 0.00 |
| ATOM | 1092 | C | LYS | 931 | 2.342 | 33.679 | 30.832 | 1.00 | 18.17 |
| ATOM | 1093 | O | LYS | 931 | 2.078 | 34.713 | 30.219 | 1.00 | 17.81 |
| ATOM | 1094 | N | ARG | 932 | 1.598 | 32.613 | 30.519 | 1.00 | 19.80 |
| ATOM | 1095 | H | ARG | 932 | 1.725 | 31.788 | 31.036 | 1.00 | 0.00 |
| ATOM | 1096 | CA | ARG | 932 | 0.530 | 32.649 | 29.447 | 1.00 | 19.95 |
| ATOM | 1097 | CB | ARG | 932 | −0.223 | 31.255 | 29.441 | 1.00 | 17.89 |
| ATOM | 1098 | CG | ARG | 932 | 0.708 | 30.061 | 29.192 | 1.00 | 16.96 |
| ATOM | 1099 | CD | ARG | 932 | 0.935 | 29.804 | 27.715 | 1.00 | 13.47 |
| ATOM | 1100 | NE | ARG | 932 | 1.815 | 28.653 | 27.598 | 1.00 | 12.87 |
| ATOM | 1101 | HE | ARG | 932 | 2.779 | 28.794 | 27.680 | 1.00 | 0.00 |
| ATOM | 1102 | CZ | ARG | 932 | 1.350 | 27.409 | 27.382 | 1.00 | 17.01 |
| ATOM | 1103 | NH1 | ARG | 932 | 2.191 | 26.390 | 27.258 | 1.00 | 13.67 |
| ATOM | 1104 | HH11 | ARG | 932 | 3.175 | 26.549 | 27.332 | 1.00 | 0.00 |
| ATOM | 1105 | HH12 | ARG | 932 | 1.843 | 25.469 | 27.085 | 1.00 | 0.00 |
| ATOM | 1106 | NH2 | ARG | 932 | 0.047 | 27.112 | 27.245 | 1.00 | 19.64 |
| ATOM | 1107 | HH21 | ARG | 932 | −0.634 | 27.843 | 27.299 | 1.00 | 0.00 |
| ATOM | 1108 | HH22 | ARG | 932 | −0.243 | 26.172 | 27.075 | 1.00 | 0.00 |
| ATOM | 1109 | C | ARG | 932 | −0.508 | 33.817 | 29.651 | 1.00 | 18.00 |
| ATOM | 1110 | O | ARG | 932 | −0.992 | 34.449 | 28.736 | 1.00 | 16.73 |
| ATOM | 1111 | N | ASN | 933 | −0.932 | 34.093 | 30.875 | 1.00 | 18.81 |
| ATOM | 1112 | H | ASN | 933 | −0.606 | 33.495 | 31.592 | 1.00 | 0.00 |
| ATOM | 1113 | CA | ASN | 933 | −1.885 | 35.185 | 31.143 | 1.00 | 20.57 |
| ATOM | 1114 | CB | ASN | 933 | −2.764 | 34.760 | 32.338 | 1.00 | 21.86 |
| ATOM | 1115 | CG | ASN | 933 | −4.041 | 35.597 | 32.460 | 1.00 | 25.01 |
| ATOM | 1116 | OD1 | ASN | 933 | −4.730 | 35.909 | 31.498 | 1.00 | 24.61 |
| ATOM | 1117 | ND2 | ASN | 933 | −4.508 | 35.912 | 33.640 | 1.00 | 26.58 |
| ATOM | 1118 | HD21 | ASN | 933 | −4.115 | 35.691 | 34.499 | 1.00 | 0.00 |
| ATOM | 1119 | HD22 | ASN | 933 | −5.320 | 36.438 | 33.627 | 1.00 | 0.00 |
| ATOM | 1120 | C | ASN | 933 | −1.145 | 36.500 | 31.427 | 1.00 | 21.84 |
| ATOM | 1121 | O | ASN | 933 | −1.734 | 37.454 | 31.956 | 1.00 | 20.90 |
| ATOM | 1122 | N | GLU | 934 | 0.165 | 36.542 | 31.083 | 1.00 | 20.33 |
| ATOM | 1123 | H | GLU | 934 | 0.531 | 35.747 | 30.642 | 1.00 | 0.00 |
| ATOM | 1124 | CA | GLU | 934 | 1.026 | 37.715 | 31.281 | 1.00 | 18.56 |
| ATOM | 1125 | CB | GLU | 934 | 1.798 | 37.567 | 32.524 | 1.00 | 19.93 |
| ATOM | 1126 | CG | GLU | 934 | 0.912 | 37.627 | 33.705 | 1.00 | 23.62 |
| ATOM | 1127 | CD | GLU | 934 | 1.633 | 37.136 | 34.950 | 1.00 | 29.00 |
| ATOM | 1128 | OE1 | GLU | 934 | 0.960 | 36.521 | 35.759 | 1.00 | 35.09 |
| ATOM | 1129 | OE2 | GLU | 934 | 2.816 | 37.348 | 35.163 | 1.00 | 29.49 |
| ATOM | 1130 | C | GLU | 934 | 2.014 | 37.875 | 30.161 | 1.00 | 18.60 |
| ATOM | 1131 | O | GLU | 934 | 3.208 | 38.127 | 30.366 | 1.00 | 18.66 |
| ATOM | 1132 | N | PHE | 935 | 1.476 | 37.767 | 28.958 | 1.00 | 15.95 |
| ATOM | 1133 | H | PHE | 935 | 0.510 | 37.621 | 28.898 | 1.00 | 0.00 |
| ATOM | 1134 | CA | PHE | 935 | 2.231 | 37.906 | 27.720 | 1.00 | 17.34 |
| ATOM | 1135 | CB | PHE | 935 | 2.562 | 36.511 | 27.103 | 1.00 | 18.67 |
| ATOM | 1136 | CG | PHE | 935 | 3.208 | 36.571 | 25.748 | 1.00 | 17.20 |
| ATOM | 1137 | CD1 | PHE | 935 | 2.430 | 36.619 | 24.577 | 1.00 | 17.74 |
| ATOM | 1138 | CD2 | PHE | 935 | 4.595 | 36.619 | 25.655 | 1.00 | 15.38 |
| ATOM | 1139 | CE1 | PHE | 935 | 3.072 | 36.708 | 23.340 | 1.00 | 17.84 |
| ATOM | 1140 | CE2 | PHE | 935 | 5.250 | 36.704 | 24.442 | 1.00 | 13.41 |
| ATOM | 1141 | CZ | PHE | 935 | 4.495 | 36.751 | 23.275 | 1.00 | 17.93 |
| ATOM | 1142 | C | PHE | 935 | 1.466 | 38.715 | 26.695 | 1.00 | 18.59 |
| ATOM | 1143 | O | PHE | 935 | 0.260 | 38.622 | 26.606 | 1.00 | 21.65 |
| ATOM | 1144 | N | VAL | 936 | 2.171 | 39.492 | 25.915 | 1.00 | 21.37 |
| ATOM | 1145 | H | VAL | 936 | 3.135 | 39.546 | 26.112 | 1.00 | 0.00 |
| ATOM | 1146 | CA | VAL | 936 | 1.642 | 40.343 | 24.839 | 1.00 | 24.92 |
| ATOM | 1147 | CB | VAL | 936 | 1.411 | 41.836 | 25.218 | 1.00 | 28.41 |
| ATOM | 1148 | CG1 | VAL | 936 | 0.048 | 41.900 | 25.771 | 1.00 | 30.85 |
| ATOM | 1149 | CG2 | VAL | 936 | 2.379 | 42.420 | 26.284 | 1.00 | 30.52 |
| ATOM | 1150 | C | VAL | 936 | 2.727 | 40.391 | 23.789 | 1.00 | 24.98 |
| ATOM | 1151 | O | VAL | 936 | 3.902 | 40.488 | 24.141 | 1.00 | 23.60 |
| ATOM | 1152 | N | PRO | 937 | 2.391 | 40.305 | 22.499 | 1.00 | 27.34 |
| ATOM | 1153 | CD | PRO | 937 | 1.068 | 40.308 | 21.903 | 1.00 | 27.39 |
| ATOM | 1154 | CA | PRO | 937 | 3.436 | 40.202 | 21.464 | 1.00 | 29.07 |
| ATOM | 1155 | CB | PRO | 937 | 2.751 | 40.029 | 20.120 | 1.00 | 27.78 |
| ATOM | 1156 | CG | PRO | 937 | 1.395 | 39.532 | 20.598 | 1.00 | 29.75 |
| ATOM | 1157 | C | PRO | 937 | 4.289 | 41.441 | 21.477 | 1.00 | 31.82 |
| ATOM | 1158 | O | PRO | 937 | 5.513 | 41.363 | 21.369 | 1.00 | 33.35 |
| ATOM | 1159 | N | TYR | 938 | 3.624 | 42.572 | 21.611 | 1.00 | 33.92 |
| ATOM | 1160 | H | TYR | 938 | 2.664 | 42.574 | 21.765 | 1.00 | 0.00 |
| ATOM | 1161 | CA | TYR | 938 | 4.254 | 43.902 | 21.670 | 1.00 | 38.16 |
| ATOM | 1162 | CB | TYR | 938 | 3.940 | 44.812 | 20.454 | 1.00 | 38.30 |
| ATOM | 1163 | C | TYR | 938 | 3.598 | 44.565 | 22.849 | 1.00 | 42.36 |
| ATOM | 1164 | O | TYR | 938 | 2.358 | 44.421 | 23.019 | 1.00 | 42.50 |
| ATOM | 1166 | CB | ASP | 998 | 6.690 | 42.019 | 35.547 | 1.00 | 39.13 |
| ATOM | 1167 | C | ASP | 998 | 5.334 | 41.514 | 33.465 | 1.00 | 35.64 |
| ATOM | 1168 | O | ASP | 998 | 5.385 | 40.318 | 33.719 | 1.00 | 35.92 |
| ATOM | 1169 | HT1 | ASP | 998 | 3.964 | 41.725 | 35.584 | 1.00 | 0.00 |
| ATOM | 1170 | HT2 | ASP | 998 | 3.576 | 43.213 | 34.861 | 1.00 | 0.00 |
| ATOM | 1171 | N | ASP | 998 | 4.311 | 42.689 | 35.376 | 1.00 | 39.83 |
| ATOM | 1172 | HT3 | ASP | 998 | 4.509 | 43.156 | 36.286 | 1.00 | 0.00 |
| ATOM | 1173 | CA | ASP | 998 | 5.571 | 42.536 | 34.595 | 1.00 | 36.70 |
| ATOM | 1174 | N | PHE | 999 | 4.818 | 41.967 | 32.321 | 1.00 | 32.22 |
| ATOM | 1175 | H | PHE | 999 | 4.713 | 42.926 | 32.164 | 1.00 | 0.00 |
| ATOM | 1176 | CA | PHE | 999 | 4.516 | 41.089 | 31.177 | 1.00 | 28.20 |
| ATOM | 1177 | CB | PHE | 999 | 3.520 | 41.824 | 30.290 | 1.00 | 28.55 |
| ATOM | 1178 | CG | PHE | 999 | 2.131 | 41.527 | 30.619 | 1.00 | 30.84 |
| ATOM | 1179 | CD1 | PHE | 999 | 1.267 | 41.319 | 29.530 | 1.00 | 32.04 |
| ATOM | 1180 | CD2 | PHE | 999 | 1.665 | 41.464 | 31.945 | 1.00 | 30.83 |
| ATOM | 1181 | CE1 | PHE | 999 | −0.092 | 41.032 | 29.756 | 1.00 | 32.75 |
| ATOM | 1182 | CE2 | PHE | 999 | 0.319 | 41.181 | 32.209 | 1.00 | 32.88 |
| ATOM | 1183 | CZ | PHE | 999 | −0.549 | 40.980 | 31.096 | 1.00 | 35.12 |
| ATOM | 1184 | C | PHE | 999 | 5.665 | 40.616 | 30.292 | 1.00 | 26.86 |
| ATOM | 1185 | O | PHE | 999 | 6.642 | 41.342 | 30.101 | 1.00 | 27.99 |
| ATOM | 1186 | N | LEU | 1000 | 5.593 | 39.419 | 29.754 | 1.00 | 21.61 |
| ATOM | 1187 | H | LEU | 1000 | 4.849 | 38.831 | 29.983 | 1.00 | 0.00 |
| ATOM | 1188 | CA | LEU | 1000 | 6.599 | 38.982 | 28.812 | 1.00 | 18.46 |
| ATOM | 1189 | CB | LEU | 1000 | 6.674 | 37.443 | 28.675 | 1.00 | 19.96 |
| ATOM | 1190 | CG | LEU | 1000 | 7.453 | 36.675 | 29.673 | 1.00 | 19.93 |
| ATOM | 1191 | CD1 | LEU | 1000 | 7.219 | 35.218 | 29.387 | 1.00 | 19.55 |
| ATOM | 1192 | CD2 | LEU | 1000 | 8.948 | 37.049 | 29.604 | 1.00 | 20.60 |
| ATOM | 1193 | C | LEU | 1000 | 6.112 | 39.509 | 27.482 | 1.00 | 16.99 |
| ATOM | 1194 | O | LEU | 1000 | 4.905 | 39.629 | 27.243 | 1.00 | 13.80 |
| ATOM | 1195 | N | THR | 1001 | 7.008 | 39.681 | 26.556 | 1.00 | 16.35 |
| ATOM | 1196 | H | THR | 1001 | 7.937 | 39.473 | 26.751 | 1.00 | 0.00 |
| ATOM | 1197 | CA | THR | 1001 | 6.626 | 40.141 | 25.225 | 1.00 | 18.34 |
| ATOM | 1198 | CB | THR | 1001 | 6.887 | 41.659 | 25.020 | 1.00 | 17.93 |
| ATOM | 1199 | OG1 | THR | 1001 | 8.284 | 41.853 | 25.098 | 1.00 | 19.57 |
| ATOM | 1200 | HG1 | THR | 1001 | 8.285 | 42.805 | 25.279 | 1.00 | 0.00 |
| ATOM | 1201 | CG2 | THR | 1001 | 6.238 | 42.556 | 26.046 | 1.00 | 15.57 |
| ATOM | 1202 | C | THR | 1001 | 7.451 | 39.383 | 24.206 | 1.00 | 18.91 |
| ATOM | 1203 | O | THR | 1001 | 8.369 | 38.610 | 24.559 | 1.00 | 20.39 |
| ATOM | 1204 | N | LEU | 1002 | 7.192 | 39.522 | 22.926 | 1.00 | 18.00 |
| ATOM | 1205 | H | LEU | 1002 | 6.461 | 40.121 | 22.641 | 1.00 | 0.00 |
| ATOM | 1206 | CA | LEU | 1002 | 8.001 | 38.828 | 21.909 | 1.00 | 20.19 |
| ATOM | 1207 | CB | LEU | 1002 | 7.425 | 39.207 | 20.586 | 1.00 | 21.63 |
| ATOM | 1208 | CG | LEU | 1002 | 7.765 | 38.418 | 19.406 | 1.00 | 21.84 |
| ATOM | 1209 | CD1 | LEU | 1002 | 7.796 | 36.870 | 19.558 | 1.00 | 20.92 |
| ATOM | 1210 | CD2 | LEU | 1002 | 6.632 | 38.894 | 18.488 | 1.00 | 23.46 |
| ATOM | 1211 | C | LEU | 1002 | 9.493 | 39.230 | 21.992 | 1.00 | 20.92 |
| ATOM | 1212 | O | LEU | 1002 | 10.433 | 38.445 | 21.770 | 1.00 | 21.44 |
| ATOM | 1213 | N | GLU | 1003 | 9.749 | 40.501 | 22.309 | 1.00 | 18.64 |
| ATOM | 1214 | H | GLU | 1003 | 8.975 | 41.111 | 22.335 | 1.00 | 0.00 |
| ATOM | 1215 | CA | GLU | 1003 | 11.134 | 40.936 | 22.454 | 1.00 | 17.74 |
| ATOM | 1216 | CB | GLU | 1003 | 11.116 | 42.306 | 22.962 | 1.00 | 19.96 |
| ATOM | 1217 | CG | GLU | 1003 | 12.490 | 42.843 | 22.601 | 1.00 | 23.83 |
| ATOM | 1218 | CD | GLU | 1003 | 12.703 | 44.253 | 23.128 | 1.00 | 25.93 |
| ATOM | 1219 | OE1 | GLU | 1003 | 11.772 | 44.928 | 23.590 | 1.00 | 25.96 |
| ATOM | 1220 | OE2 | GLU | 1003 | 13.854 | 44.671 | 23.056 | 1.00 | 31.50 |
| ATOM | 1221 | C | GLU | 1003 | 11.869 | 40.036 | 23.476 | 1.00 | 17.42 |
| ATOM | 1222 | O | GLU | 1003 | 12.975 | 39.573 | 23.242 | 1.00 | 17.96 |
| ATOM | 1223 | N | HIS | 1004 | 11.317 | 39.838 | 24.672 | 1.00 | 16.27 |
| ATOM | 1224 | H | HIS | 1004 | 10.439 | 40.256 | 24.840 | 1.00 | 0.00 |
| ATOM | 1225 | CA | HIS | 1004 | 11.913 | 38.946 | 25.719 | 1.00 | 16.06 |
| ATOM | 1226 | C | HIS | 1004 | 12.223 | 37.575 | 25.149 | 1.00 | 14.81 |
| ATOM | 1227 | O | HIS | 1004 | 13.287 | 37.036 | 25.390 | 1.00 | 14.88 |
| ATOM | 1228 | CB | HIS | 1004 | 10.949 | 38.674 | 26.839 | 1.00 | 16.28 |
| ATOM | 1229 | CG | HIS | 1004 | 10.641 | 39.870 | 27.605 | 1.00 | 17.20 |
| ATOM | 1230 | ND1 | HIS | 1004 | 11.229 | 40.191 | 28.774 | 1.00 | 20.53 |
| ATOM | 1231 | HD1 | HIS | 1004 | 11.826 | 39.626 | 29.313 | 1.00 | 0.00 |
| ATOM | 1232 | CD2 | HIS | 1004 | 9.707 | 40.834 | 27.316 | 1.00 | 18.39 |
| ATOM | 1233 | NE2 | HIS | 1004 | 9.704 | 41.720 | 28.329 | 1.00 | 20.89 |
| ATOM | 1234 | CE1 | HIS | 1004 | 10.645 | 41.341 | 29.237 | 1.00 | 17.00 |
| ATOM | 1235 | N | LEU | 1005 | 11.235 | 36.987 | 24.443 | 1.00 | 14.93 |
| ATOM | 1236 | H | LEU | 1005 | 10.384 | 37.470 | 24.377 | 1.00 | 0.00 |
| ATOM | 1237 | CA | LEU | 1005 | 11.336 | 35.651 | 23.802 | 1.00 | 14.44 |
| ATOM | 1238 | CB | LEU | 1005 | 9.988 | 35.363 | 23.107 | 1.00 | 13.30 |
| ATOM | 1239 | CG | LEU | 1005 | 8.897 | 34.601 | 23.906 | 1.00 | 16.10 |
| ATOM | 1240 | CD1 | LEU | 1005 | 8.900 | 34.972 | 25.369 | 1.00 | 16.97 |
| ATOM | 1241 | CD2 | LEU | 1005 | 7.612 | 34.699 | 23.098 | 1.00 | 13.85 |
| ATOM | 1242 | C | LEU | 1005 | 12.485 | 35.583 | 22.829 | 1.00 | 15.62 |
| ATOM | 1243 | O | LEU | 1005 | 13.300 | 34.635 | 22.865 | 1.00 | 14.77 |
| ATOM | 1244 | N | ILE | 1006 | 12.580 | 36.561 | 21.915 | 1.00 | 13.68 |
| ATOM | 1245 | H | ILE | 1006 | 11.866 | 37.230 | 21.875 | 1.00 | 0.00 |
| ATOM | 1246 | CA | ILE | 1006 | 13.728 | 36.602 | 20.969 | 1.00 | 14.26 |
| ATOM | 1247 | CB | ILE | 1006 | 13.583 | 37.714 | 19.895 | 1.00 | 14.47 |
| ATOM | 1248 | CG2 | ILE | 1006 | 14.833 | 37.712 | 18.933 | 1.00 | 12.35 |
| ATOM | 1249 | CG1 | ILE | 1006 | 12.209 | 37.500 | 19.216 | 1.00 | 18.77 |
| ATOM | 1250 | CD1 | ILE | 1006 | 11.753 | 38.601 | 18.210 | 1.00 | 19.53 |
| ATOM | 1251 | C | ILE | 1006 | 15.044 | 36.880 | 21.750 | 1.00 | 13.38 |
| ATOM | 1252 | O | ILE | 1006 | 16.106 | 36.341 | 21.454 | 1.00 | 14.54 |
| ATOM | 1253 | N | CYS | 1007 | 14.999 | 37.753 | 22.744 | 1.00 | 11.90 |
| ATOM | 1254 | H | CYS | 1007 | 14.134 | 38.172 | 22.918 | 1.00 | 0.00 |
| ATOM | 1255 | CA | CYS | 1007 | 16.173 | 38.057 | 23.549 | 1.00 | 12.20 |
| ATOM | 1256 | CB | CYS | 1007 | 15.806 | 39.138 | 24.592 | 1.00 | 13.44 |
| ATOM | 1257 | SG | CYS | 1007 | 17.313 | 39.600 | 25.503 | 1.00 | 19.83 |
| ATOM | 1258 | C | CYS | 1007 | 16.728 | 36.769 | 24.261 | 1.00 | 14.29 |
| ATOM | 1259 | O | CYS | 1007 | 17.941 | 36.438 | 24.211 | 1.00 | 12.19 |
| ATOM | 1260 | N | TYR | 1008 | 15.843 | 36.003 | 24.940 | 1.00 | 11.40 |
| ATOM | 1261 | H | TYR | 1008 | 14.903 | 36.256 | 24.969 | 1.00 | 0.00 |
| ATOM | 1262 | CA | TYR | 1008 | 16.303 | 34.775 | 25.615 | 1.00 | 12.66 |
| ATOM | 1263 | CB | TYR | 1008 | 15.126 | 34.121 | 26.302 | 1.00 | 11.34 |
| ATOM | 1264 | CG | TYR | 1008 | 14.416 | 34.903 | 27.341 | 1.00 | 13.82 |
| ATOM | 1265 | CD1 | TYR | 1008 | 13.060 | 34.568 | 27.574 | 1.00 | 12.39 |
| ATOM | 1266 | CE1 | TYR | 1008 | 12.327 | 35.232 | 28.582 | 1.00 | 12.54 |
| ATOM | 1267 | CD2 | TYR | 1008 | 15.039 | 35.910 | 28.139 | 1.00 | 15.02 |
| ATOM | 1268 | CE2 | TYR | 1008 | 14.315 | 36.583 | 29.149 | 1.00 | 14.94 |
| ATOM | 1269 | CZ | TYR | 1008 | 12.971 | 36.222 | 29.352 | 1.00 | 15.49 |
| ATOM | 1270 | OH | TYR | 1008 | 12.251 | 36.850 | 30.320 | 1.00 | 16.38 |
| ATOM | 1271 | HH | TYR | 1008 | 11.368 | 36.485 | 30.351 | 1.00 | 0.00 |
| ATOM | 1272 | C | TYR | 1008 | 16.929 | 33.778 | 24.595 | 1.00 | 12.04 |
| ATOM | 1273 | O | TYR | 1008 | 17.971 | 33.130 | 24.812 | 1.00 | 11.06 |
| ATOM | 1274 | N | SER | 1009 | 16.267 | 33.697 | 23.414 | 1.00 | 11.13 |
| ATOM | 1275 | H | SER | 1009 | 15.406 | 34.160 | 23.306 | 1.00 | 0.00 |
| ATOM | 1276 | CA | SER | 1009 | 16.731 | 32.832 | 22.340 | 1.00 | 12.56 |
| ATOM | 1277 | CB | SER | 1009 | 15.805 | 32.956 | 21.091 | 1.00 | 13.07 |
| ATOM | 1278 | OG | SER | 1009 | 14.457 | 32.579 | 21.394 | 1.00 | 11.97 |
| ATOM | 1279 | HG | SER | 1009 | 13.922 | 32.692 | 20.606 | 1.00 | 0.00 |
| ATOM | 1280 | C | SER | 1009 | 18.136 | 33.205 | 21.917 | 1.00 | 11.19 |
| ATOM | 1281 | O | SER | 1009 | 19.011 | 32.370 | 21.732 | 1.00 | 12.15 |
| ATOM | 1282 | N | PHE | 1010 | 18.380 | 34.474 | 21.626 | 1.00 | 12.64 |
| ATOM | 1283 | H | PHE | 1010 | 17.629 | 35.105 | 21.669 | 1.00 | 0.00 |
| ATOM | 1284 | CA | PHE | 1010 | 19.735 | 34.989 | 21.224 | 1.00 | 13.28 |
| ATOM | 1285 | CB | PHE | 1010 | 19.579 | 36.546 | 20.882 | 1.00 | 15.81 |
| ATOM | 1286 | CG | PHE | 1010 | 20.859 | 37.347 | 20.823 | 1.00 | 13.63 |
| ATOM | 1287 | CD1 | PHE | 1010 | 21.234 | 38.189 | 21.894 | 1.00 | 14.37 |
| ATOM | 1288 | CD2 | PHE | 1010 | 21.699 | 37.246 | 19.706 | 1.00 | 13.09 |
| ATOM | 1289 | CE1 | PHE | 1010 | 22.439 | 38.918 | 21.850 | 1.00 | 13.98 |
| ATOM | 1290 | CE2 | PHE | 1010 | 22.911 | 37.979 | 19.654 | 1.00 | 13.81 |
| ATOM | 1291 | CZ | PHE | 1010 | 23.267 | 38.812 | 20.734 | 1.00 | 13.96 |
| ATOM | 1292 | C | PHE | 1010 | 20.794 | 34.720 | 22.380 | 1.00 | 12.60 |
| ATOM | 1293 | O | PHE | 1010 | 21.936 | 34.298 | 22.152 | 1.00 | 12.76 |
| ATOM | 1294 | N | GLN | 1011 | 20.436 | 35.009 | 23.647 | 1.00 | 12.89 |
| ATOM | 1295 | H | GLN | 1011 | 19.561 | 35.418 | 23.783 | 1.00 | 0.00 |
| ATOM | 1296 | CA | GLN | 1011 | 21.324 | 34.734 | 24.784 | 1.00 | 11.62 |
| ATOM | 1297 | CB | GLN | 1011 | 20.656 | 35.163 | 26.041 | 1.00 | 10.75 |
| ATOM | 1298 | CG | GLN | 1011 | 20.592 | 36.674 | 26.214 | 1.00 | 11.35 |
| ATOM | 1299 | CD | GLN | 1011 | 20.036 | 36.897 | 27.629 | 1.00 | 15.30 |
| ATOM | 1300 | OE1 | GLN | 1011 | 20.711 | 36.668 | 28.641 | 1.00 | 11.72 |
| ATOM | 1301 | NE2 | GLN | 1011 | 18.762 | 37.324 | 27.747 | 1.00 | 13.24 |
| ATOM | 1302 | HE21 | GLN | 1011 | 18.204 | 37.489 | 26.968 | 1.00 | 0.00 |
| ATOM | 1303 | HE22 | GLN | 1011 | 18.407 | 37.473 | 28.644 | 1.00 | 0.00 |
| ATOM | 1304 | C | GLN | 1011 | 21.663 | 33.243 | 24.874 | 1.00 | 12.50 |
| ATOM | 1305 | O | GLN | 1011 | 22.828 | 32.883 | 25.099 | 1.00 | 14.54 |
| ATOM | 1306 | N | VAL | 1012 | 20.712 | 32.309 | 24.694 | 1.00 | 11.33 |
| ATOM | 1307 | H | VAL | 1012 | 19.785 | 32.575 | 24.556 | 1.00 | 0.00 |
| ATOM | 1308 | CA | VAL | 1012 | 21.112 | 30.903 | 24.777 | 1.00 | 11.64 |
| ATOM | 1309 | CB | VAL | 1012 | 19.871 | 29.985 | 24.807 | 1.00 | 11.09 |
| ATOM | 1310 | CG1 | VAL | 1012 | 20.383 | 28.530 | 24.771 | 1.00 | 12.97 |
| ATOM | 1311 | CG2 | VAL | 1012 | 19.011 | 30.254 | 26.055 | 1.00 | 7.98 |
| ATOM | 1312 | C | VAL | 1012 | 22.015 | 30.585 | 23.590 | 1.00 | 11.05 |
| ATOM | 1313 | O | VAL | 1012 | 23.011 | 29.865 | 23.722 | 1.00 | 11.67 |
| ATOM | 1314 | N | ALA | 1013 | 21.769 | 31.167 | 22.409 | 1.00 | 11.56 |
| ATOM | 1315 | H | ALA | 1013 | 20.977 | 31.745 | 22.321 | 1.00 | 0.00 |
| ATOM | 1316 | CA | ALA | 1013 | 22.648 | 30.926 | 21.233 | 1.00 | 11.59 |
| ATOM | 1317 | CB | ALA | 1013 | 22.072 | 31.584 | 19.978 | 1.00 | 11.37 |
| ATOM | 1318 | C | ALA | 1013 | 24.042 | 31.499 | 21.516 | 1.00 | 11.25 |
| ATOM | 1319 | O | ALA | 1013 | 25.073 | 30.917 | 21.153 | 1.00 | 12.88 |
| ATOM | 1320 | N | LYS | 1014 | 24.131 | 32.637 | 22.203 | 1.00 | 13.06 |
| ATOM | 1321 | H | LYS | 1014 | 23.307 | 33.090 | 22.468 | 1.00 | 0.00 |
| ATOM | 1322 | CA | LYS | 1014 | 25.459 | 33.218 | 22.594 | 1.00 | 12.82 |
| ATOM | 1323 | CB | LYS | 1014 | 25.280 | 34.594 | 23.289 | 1.00 | 13.37 |
| ATOM | 1324 | CG | LYS | 1014 | 24.908 | 35.665 | 22.309 | 1.00 | 14.80 |
| ATOM | 1325 | CD | LYS | 1014 | 26.140 | 36.101 | 21.537 | 1.00 | 18.01 |
| ATOM | 1326 | CE | LYS | 1014 | 26.723 | 37.350 | 22.217 | 1.00 | 21.41 |
| ATOM | 1327 | NZ | LYS | 1014 | 27.893 | 37.817 | 21.449 | 1.00 | 18.85 |
| ATOM | 1328 | HZ1 | LYS | 1014 | 27.645 | 37.866 | 20.440 | 1.00 | 0.00 |
| ATOM | 1329 | HZ2 | LYS | 1014 | 28.680 | 37.149 | 21.570 | 1.00 | 0.00 |
| ATOM | 1330 | HZ3 | LYS | 1014 | 28.175 | 38.758 | 21.787 | 1.00 | 0.00 |
| ATOM | 1331 | C | LYS | 1014 | 26.191 | 32.263 | 23.583 | 1.00 | 12.28 |
| ATOM | 1332 | O | LYS | 1014 | 27.381 | 31.949 | 23.449 | 1.00 | 11.19 |
| ATOM | 1333 | N | GLY | 1015 | 25.477 | 31.754 | 24.597 | 1.00 | 13.00 |
| ATOM | 1334 | H | GLY | 1015 | 24.551 | 32.056 | 24.713 | 1.00 | 0.00 |
| ATOM | 1335 | CA | GLY | 1015 | 26.102 | 30.801 | 25.582 | 1.00 | 11.46 |
| ATOM | 1336 | C | GLY | 1015 | 26.558 | 29.517 | 24.882 | 1.00 | 11.28 |
| ATOM | 1337 | O | GLY | 1015 | 27.597 | 28.977 | 25.184 | 1.00 | 8.45 |
| ATOM | 1338 | N | MET | 1016 | 25.765 | 28.965 | 23.951 | 1.00 | 10.94 |
| ATOM | 1339 | H | MET | 1016 | 24.883 | 29.370 | 23.824 | 1.00 | 0.00 |
| ATOM | 1340 | CA | MET | 1016 | 26.139 | 27.742 | 23.224 | 1.00 | 10.73 |
| ATOM | 1341 | CB | MET | 1016 | 24.874 | 27.219 | 22.543 | 1.00 | 10.81 |
| ATOM | 1342 | CG | MET | 1016 | 23.763 | 26.627 | 23.416 | 1.00 | 7.81 |
| ATOM | 1343 | SD | MET | 1016 | 24.457 | 25.258 | 24.365 | 1.00 | 14.59 |
| ATOM | 1344 | CE | MET | 1016 | 25.290 | 24.149 | 23.229 | 1.00 | 14.27 |
| ATOM | 1345 | C | MET | 1016 | 27.301 | 27.993 | 22.215 | 1.00 | 12.12 |
| ATOM | 1346 | O | MET | 1016 | 28.153 | 27.136 | 21.955 | 1.00 | 11.52 |
| ATOM | 1347 | N | GLU | 1017 | 27.353 | 29.172 | 21.574 | 1.00 | 12.11 |
| ATOM | 1348 | H | GLU | 1017 | 26.589 | 29.779 | 21.658 | 1.00 | 0.00 |
| ATOM | 1349 | CA | GLU | 1017 | 28.479 | 29.517 | 20.721 | 1.00 | 12.02 |
| ATOM | 1350 | CB | GLU | 1017 | 28.165 | 30.883 | 20.122 | 1.00 | 12.90 |
| ATOM | 1351 | CG | GLU | 1017 | 29.413 | 31.367 | 19.410 | 1.00 | 14.75 |
| ATOM | 1352 | CD | GLU | 1017 | 29.238 | 32.772 | 18.847 | 1.00 | 19.04 |
| ATOM | 1353 | OE1 | GLU | 1017 | 28.894 | 33.684 | 19.643 | 1.00 | 17.11 |
| ATOM | 1354 | OE2 | GLU | 1017 | 29.516 | 32.940 | 17.650 | 1.00 | 17.41 |
| ATOM | 1355 | C | GLU | 1017 | 29.718 | 29.521 | 21.630 | 1.00 | 12.67 |
| ATOM | 1356 | O | GLU | 1017 | 30.797 | 29.096 | 21.236 | 1.00 | 14.11 |
| ATOM | 1357 | N | PHE | 1018 | 29.619 | 30.040 | 22.866 | 1.00 | 13.88 |
| ATOM | 1358 | H | PHE | 1018 | 28.769 | 30.457 | 23.123 | 1.00 | 0.00 |
| ATOM | 1359 | CA | PHE | 1018 | 30.719 | 30.047 | 23.812 | 1.00 | 12.32 |
| ATOM | 1360 | CB | PHE | 1018 | 30.303 | 30.798 | 25.041 | 1.00 | 11.54 |
| ATOM | 1361 | CG | PHE | 1018 | 31.356 | 30.699 | 26.113 | 1.00 | 14.88 |
| ATOM | 1362 | CD1 | PHE | 1018 | 31.155 | 29.894 | 27.258 | 1.00 | 15.65 |
| ATOM | 1363 | CD2 | PHE | 1018 | 32.548 | 31.448 | 26.018 | 1.00 | 18.92 |
| ATOM | 1364 | CE1 | PHE | 1018 | 32.103 | 29.838 | 28.283 | 1.00 | 16.49 |
| ATOM | 1365 | CE2 | PHE | 1018 | 33.509 | 31.390 | 27.060 | 1.00 | 18.86 |
| ATOM | 1366 | CZ | PHE | 1018 | 33.278 | 30.589 | 28.180 | 1.00 | 16.01 |
| ATOM | 1367 | C | PHE | 1018 | 31.058 | 28.610 | 24.164 | 1.00 | 15.23 |
| ATOM | 1368 | O | PHE | 1018 | 32.223 | 28.179 | 24.094 | 1.00 | 16.64 |
| ATOM | 1369 | N | LEU | 1019 | 30.077 | 27.763 | 24.511 | 1.00 | 15.97 |
| ATOM | 1370 | H | LEU | 1019 | 29.157 | 28.089 | 24.600 | 1.00 | 0.00 |
| ATOM | 1371 | CA | LEU | 1019 | 30.403 | 26.366 | 24.832 | 1.00 | 14.65 |
| ATOM | 1372 | CB | LEU | 1019 | 29.141 | 25.512 | 25.011 | 1.00 | 18.20 |
| ATOM | 1373 | CG | LEU | 1019 | 28.647 | 25.390 | 26.419 | 1.00 | 26.16 |
| ATOM | 1374 | CD1 | LEU | 1019 | 27.419 | 24.450 | 26.480 | 1.00 | 26.09 |
| ATOM | 1375 | CD2 | LEU | 1019 | 29.799 | 24.819 | 27.256 | 1.00 | 26.37 |
| ATOM | 1376 | C | LEU | 1019 | 31.211 | 25.720 | 23.720 | 1.00 | 14.84 |
| ATOM | 1377 | O | LEU | 1019 | 32.226 | 25.066 | 23.953 | 1.00 | 14.62 |
| ATOM | 1378 | N | ALA | 1020 | 30.739 | 25.870 | 22.479 | 1.00 | 13.50 |
| ATOM | 1379 | H | ALA | 1020 | 29.910 | 26.382 | 22.364 | 1.00 | 0.00 |
| ATOM | 1380 | CA | ALA | 1020 | 31.386 | 25.272 | 21.295 | 1.00 | 15.91 |
| ATOM | 1381 | CB | ALA | 1020 | 30.519 | 25.509 | 19.974 | 1.00 | 11.47 |
| ATOM | 1382 | C | ALA | 1020 | 32.810 | 25.827 | 21.113 | 1.00 | 15.57 |
| ATOM | 1383 | O | ALA | 1020 | 33.708 | 25.079 | 20.658 | 1.00 | 16.40 |
| ATOM | 1384 | N | SER | 1021 | 33.022 | 27.097 | 21.478 | 1.00 | 14.16 |
| ATOM | 1385 | H | SER | 1021 | 32.285 | 27.641 | 21.832 | 1.00 | 0.00 |
| ATOM | 1386 | CA | SER | 1021 | 34.396 | 27.669 | 21.378 | 1.00 | 15.64 |
| ATOM | 1387 | CB | SER | 1021 | 34.382 | 29.185 | 21.609 | 1.00 | 12.50 |
| ATOM | 1388 | OG | SER | 1021 | 34.231 | 29.564 | 22.960 | 1.00 | 15.14 |
| ATOM | 1389 | HG | SER | 1021 | 34.432 | 30.497 | 23.042 | 1.00 | 0.00 |
| ATOM | 1390 | C | SER | 1021 | 35.376 | 26.999 | 22.377 | 1.00 | 18.10 |
| ATOM | 1391 | O | SER | 1021 | 36.601 | 27.028 | 22.206 | 1.00 | 18.25 |
| ATOM | 1392 | N | ARG | 1022 | 34.853 | 26.403 | 23.471 | 1.00 | 17.77 |
| ATOM | 1393 | H | ARG | 1022 | 33.894 | 26.560 | 23.634 | 1.00 | 0.00 |
| ATOM | 1394 | CA | ARG | 1022 | 35.667 | 25.662 | 24.493 | 1.00 | 18.79 |
| ATOM | 1395 | CB | ARG | 1022 | 35.040 | 25.793 | 25.893 | 1.00 | 16.78 |
| ATOM | 1396 | CG | ARG | 1022 | 34.776 | 27.271 | 26.151 | 1.00 | 15.45 |
| ATOM | 1397 | CD | ARG | 1022 | 35.793 | 27.876 | 27.037 | 1.00 | 22.99 |
| ATOM | 1398 | NE | ARG | 1022 | 36.998 | 28.059 | 26.353 | 1.00 | 23.94 |
| ATOM | 1399 | HE | ARG | 1022 | 36.946 | 28.482 | 25.471 | 1.00 | 0.00 |
| ATOM | 1400 | CZ | ARG | 1022 | 38.212 | 27.712 | 26.762 | 1.00 | 27.81 |
| ATOM | 1401 | NH1 | ARG | 1022 | 39.183 | 27.999 | 25.923 | 1.00 | 31.74 |
| ATOM | 1402 | HH11 | ARG | 1022 | 38.974 | 28.439 | 25.049 | 1.00 | 0.00 |
| ATOM | 1403 | HH12 | ARG | 1022 | 40.131 | 27.770 | 26.149 | 1.00 | 0.00 |
| ATOM | 1404 | NH2 | ARG | 1022 | 38.604 | 27.067 | 27.850 | 1.00 | 27.41 |
| ATOM | 1405 | HH21 | ARG | 1022 | 37.927 | 26.766 | 28.513 | 1.00 | 0.00 |
| ATOM | 1406 | HH22 | ARG | 1022 | 39.578 | 26.896 | 28.010 | 1.00 | 0.00 |
| ATOM | 1407 | C | ARG | 1022 | 35.753 | 24.182 | 24.147 | 1.00 | 19.64 |
| ATOM | 1408 | O | ARG | 1022 | 36.071 | 23.336 | 24.978 | 1.00 | 19.39 |
| ATOM | 1409 | N | LYS | 1023 | 35.275 | 23.865 | 22.937 | 1.00 | 22.30 |
| ATOM | 1410 | H | LYS | 1023 | 35.017 | 24.626 | 22.381 | 1.00 | 0.00 |
| ATOM | 1411 | CA | LYS | 1023 | 35.171 | 22.556 | 22.307 | 1.00 | 23.56 |
| ATOM | 1412 | CB | LYS | 1023 | 36.579 | 21.970 | 22.060 | 1.00 | 29.00 |
| ATOM | 1413 | CG | LYS | 1023 | 37.388 | 22.898 | 21.206 | 1.00 | 33.55 |
| ATOM | 1414 | CD | LYS | 1023 | 38.796 | 22.334 | 21.049 | 1.00 | 40.54 |
| ATOM | 1415 | CE | LYS | 1023 | 39.841 | 23.432 | 20.610 | 1.00 | 43.78 |
| ATOM | 1416 | NZ | LYS | 1023 | 39.356 | 24.159 | 19.409 | 1.00 | 47.46 |
| ATOM | 1417 | HZ1 | LYS | 1023 | 38.356 | 24.403 | 19.557 | 1.00 | 0.00 |
| ATOM | 1418 | HZ2 | LYS | 1023 | 39.426 | 23.520 | 18.591 | 1.00 | 0.00 |
| ATOM | 1419 | HZ3 | LYS | 1023 | 39.902 | 25.027 | 19.237 | 1.00 | 0.00 |
| ATOM | 1420 | C | LYS | 1023 | 34.365 | 21.598 | 23.187 | 1.00 | 24.84 |
| ATOM | 1421 | O | LYS | 1023 | 34.690 | 20.413 | 23.376 | 1.00 | 23.68 |
| ATOM | 1422 | N | CYS | 1024 | 33.264 | 22.101 | 23.752 | 1.00 | 21.28 |
| ATOM | 1423 | H | CYS | 1024 | 33.082 | 23.059 | 23.647 | 1.00 | 0.00 |
| ATOM | 1424 | CA | CYS | 1024 | 32.420 | 21.254 | 24.582 | 1.00 | 18.79 |
| ATOM | 1425 | CB | CYS | 1024 | 32.309 | 21.888 | 26.003 | 1.00 | 19.38 |
| ATOM | 1426 | SG | CYS | 1024 | 33.850 | 21.728 | 26.983 | 1.00 | 21.65 |
| ATOM | 1427 | C | CYS | 1024 | 31.041 | 21.078 | 23.961 | 1.00 | 19.89 |
| ATOM | 1428 | O | CYS | 1024 | 30.653 | 21.890 | 23.123 | 1.00 | 21.64 |
| ATOM | 1429 | N | ILE | 1025 | 30.282 | 20.015 | 24.245 | 1.00 | 18.76 |
| ATOM | 1430 | H | ILE | 1025 | 30.637 | 19.263 | 24.754 | 1.00 | 0.00 |
| ATOM | 1431 | CA | ILE | 1025 | 28.911 | 19.952 | 23.709 | 1.00 | 18.28 |
| ATOM | 1432 | CB | ILE | 1025 | 28.739 | 18.845 | 22.634 | 1.00 | 19.24 |
| ATOM | 1433 | CG2 | ILE | 1025 | 29.973 | 18.821 | 21.672 | 1.00 | 22.05 |
| ATOM | 1434 | CG1 | ILE | 1025 | 28.577 | 17.535 | 23.240 | 1.00 | 21.22 |
| ATOM | 1435 | CD1 | ILE | 1025 | 28.430 | 16.570 | 22.033 | 1.00 | 26.22 |
| ATOM | 1436 | C | ILE | 1025 | 28.011 | 19.703 | 24.943 | 1.00 | 18.37 |
| ATOM | 1437 | O | ILE | 1025 | 28.441 | 19.172 | 25.982 | 1.00 | 17.58 |
| ATOM | 1438 | N | HIS | 1026 | 26.796 | 20.177 | 24.926 | 1.00 | 14.11 |
| ATOM | 1439 | H | HIS | 1026 | 26.609 | 20.866 | 24.273 | 1.00 | 0.00 |
| ATOM | 1440 | CA | HIS | 1026 | 25.905 | 20.034 | 26.082 | 1.00 | 13.85 |
| ATOM | 1441 | C | HIS | 1026 | 25.315 | 18.666 | 26.284 | 1.00 | 15.23 |
| ATOM | 1442 | O | HIS | 1026 | 25.410 | 18.129 | 27.377 | 1.00 | 14.64 |
| ATOM | 1443 | CB | HIS | 1026 | 24.753 | 21.144 | 26.004 | 1.00 | 11.30 |
| ATOM | 1444 | CG | HIS | 1026 | 23.972 | 21.255 | 27.286 | 1.00 | 11.07 |
| ATOM | 1445 | ND1 | HIS | 1026 | 23.172 | 20.284 | 27.791 | 1.00 | 10.39 |
| ATOM | 1446 | CE1 | HIS | 1026 | 22.659 | 20.691 | 29.005 | 1.00 | 7.98 |
| ATOM | 1447 | CD2 | HIS | 1026 | 23.952 | 22.291 | 28.226 | 1.00 | 9.75 |
| ATOM | 1448 | NE2 | HIS | 1026 | 23.147 | 21.918 | 29.253 | 1.00 | 9.73 |
| ATOM | 1449 | HE2 | HIS | 1026 | 22.898 | 22.465 | 30.034 | 1.00 | 0.00 |
| ATOM | 1450 | N | ARG | 1027 | 24.775 | 18.066 | 25.217 | 1.00 | 14.54 |
| ATOM | 1451 | H | ARG | 1027 | 24.925 | 18.520 | 24.374 | 1.00 | 0.00 |
| ATOM | 1452 | CA | ARG | 1027 | 24.089 | 16.755 | 25.212 | 1.00 | 14.85 |
| ATOM | 1453 | CB | ARG | 1027 | 24.927 | 15.621 | 25.851 | 1.00 | 15.44 |
| ATOM | 1454 | CG | ARG | 1027 | 26.249 | 15.529 | 25.042 | 1.00 | 17.78 |
| ATOM | 1455 | CD | ARG | 1027 | 27.029 | 14.319 | 25.570 | 1.00 | 20.21 |
| ATOM | 1456 | NE | ARG | 1027 | 28.310 | 14.219 | 24.858 | 1.00 | 22.17 |
| ATOM | 1457 | HE | ARG | 1027 | 28.402 | 13.583 | 24.116 | 1.00 | 0.00 |
| ATOM | 1458 | CZ | ARG | 1027 | 29.366 | 14.980 | 25.186 | 1.00 | 24.11 |
| ATOM | 1459 | NH1 | ARG | 1027 | 30.455 | 14.797 | 24.470 | 1.00 | 24.72 |
| ATOM | 1460 | HH11 | ARG | 1027 | 30.486 | 14.131 | 23.725 | 1.00 | 0.00 |
| ATOM | 1461 | HH12 | ARG | 1027 | 31.261 | 15.349 | 24.678 | 1.00 | 0.00 |
| ATOM | 1462 | NH2 | ARG | 1027 | 29.386 | 15.945 | 26.124 | 1.00 | 19.73 |
| ATOM | 1463 | HH21 | ARG | 1027 | 28.546 | 16.136 | 26.625 | 1.00 | 0.00 |
| ATOM | 1464 | HH22 | ARG | 1027 | 30.218 | 16.464 | 26.317 | 1.00 | 0.00 |
| ATOM | 1465 | C | ARG | 1027 | 22.726 | 16.770 | 25.898 | 1.00 | 15.15 |
| ATOM | 1466 | O | ARG | 1027 | 22.031 | 15.783 | 25.796 | 1.00 | 17.29 |
| ATOM | 1467 | N | ASP | 1028 | 22.247 | 17.819 | 26.563 | 1.00 | 12.98 |
| ATOM | 1468 | H | ASP | 1028 | 22.827 | 18.590 | 26.700 | 1.00 | 0.00 |
| ATOM | 1469 | CA | ASP | 1028 | 20.867 | 17.808 | 27.114 | 1.00 | 13.81 |
| ATOM | 1470 | CB | ASP | 1028 | 20.884 | 17.239 | 28.554 | 1.00 | 10.92 |
| ATOM | 1471 | CG | ASP | 1028 | 19.477 | 16.877 | 29.059 | 1.00 | 13.90 |
| ATOM | 1472 | OD1 | ASP | 1028 | 19.368 | 16.564 | 30.237 | 1.00 | 14.72 |
| ATOM | 1473 | OD2 | ASP | 1028 | 18.467 | 16.903 | 28.340 | 1.00 | 14.75 |
| ATOM | 1474 | C | ASP | 1028 | 20.277 | 19.236 | 27.109 | 1.00 | 12.93 |
| ATOM | 1475 | O | ASP | 1028 | 19.652 | 19.740 | 28.076 | 1.00 | 12.95 |
| ATOM | 1476 | N | LEU | 1029 | 20.471 | 19.899 | 25.962 | 1.00 | 10.36 |
| ATOM | 1477 | H | LEU | 1029 | 20.866 | 19.426 | 25.211 | 1.00 | 0.00 |
| ATOM | 1478 | CA | LEU | 1029 | 19.978 | 21.275 | 25.846 | 1.00 | 10.75 |
| ATOM | 1479 | CB | LEU | 1029 | 20.691 | 21.949 | 24.622 | 1.00 | 9.18 |
| ATOM | 1480 | CG | LEU | 1029 | 20.249 | 23.394 | 24.452 | 1.00 | 8.50 |
| ATOM | 1481 | CD1 | LEU | 1029 | 20.725 | 24.349 | 25.604 | 1.00 | 10.27 |
| ATOM | 1482 | CD2 | LEU | 1029 | 20.921 | 23.888 | 23.172 | 1.00 | 8.31 |
| ATOM | 1483 | C | LEU | 1029 | 18.469 | 21.209 | 25.721 | 1.00 | 10.44 |
| ATOM | 1484 | O | LEU | 1029 | 17.932 | 20.471 | 24.901 | 1.00 | 10.44 |
| ATOM | 1485 | N | ALA | 1030 | 17.781 | 21.945 | 26.570 | 1.00 | 11.50 |
| ATOM | 1486 | H | ALA | 1030 | 18.328 | 22.482 | 27.188 | 1.00 | 0.00 |
| ATOM | 1487 | CA | ALA | 1030 | 16.300 | 22.007 | 26.690 | 1.00 | 11.94 |
| ATOM | 1488 | CB | ALA | 1030 | 15.721 | 20.737 | 27.361 | 1.00 | 7.80 |
| ATOM | 1489 | C | ALA | 1030 | 15.893 | 23.186 | 27.573 | 1.00 | 12.77 |
| ATOM | 1490 | O | ALA | 1030 | 16.722 | 23.650 | 28.353 | 1.00 | 13.94 |
| ATOM | 1491 | N | ALA | 1031 | 14.647 | 23.695 | 27.536 | 1.00 | 13.10 |
| ATOM | 1492 | H | ALA | 1031 | 13.984 | 23.240 | 26.991 | 1.00 | 0.00 |
| ATOM | 1493 | CA | ALA | 1031 | 14.255 | 24.832 | 28.394 | 1.00 | 11.27 |
| ATOM | 1494 | CB | ALA | 1031 | 12.788 | 25.302 | 28.123 | 1.00 | 10.90 |
| ATOM | 1495 | C | ALA | 1031 | 14.371 | 24.442 | 29.884 | 1.00 | 11.50 |
| ATOM | 1496 | O | ALA | 1031 | 14.616 | 25.324 | 30.700 | 1.00 | 11.66 |
| ATOM | 1497 | N | ARG | 1032 | 14.184 | 23.164 | 30.279 | 1.00 | 10.32 |
| ATOM | 1498 | H | ARG | 1032 | 13.911 | 22.541 | 29.578 | 1.00 | 0.00 |
| ATOM | 1499 | CA | ARG | 1032 | 14.329 | 22.714 | 31.699 | 1.00 | 10.89 |
| ATOM | 1500 | CB | ARG | 1032 | 13.946 | 21.212 | 31.778 | 1.00 | 12.39 |
| ATOM | 1501 | CG | ARG | 1032 | 14.944 | 20.267 | 31.071 | 1.00 | 10.52 |
| ATOM | 1502 | CD | ARG | 1032 | 14.915 | 18.691 | 31.274 | 1.00 | 15.19 |
| ATOM | 1503 | NE | ARG | 1032 | 14.336 | 18.407 | 30.000 | 1.00 | 21.87 |
| ATOM | 1504 | HE | ARG | 1032 | 13.410 | 18.732 | 29.967 | 1.00 | 0.00 |
| ATOM | 1505 | CZ | ARG | 1032 | 14.695 | 17.844 | 28.869 | 1.00 | 16.89 |
| ATOM | 1506 | NH1 | ARG | 1032 | 13.676 | 18.001 | 28.094 | 1.00 | 19.18 |
| ATOM | 1507 | HH11 | ARG | 1032 | 12.853 | 18.466 | 28.423 | 1.00 | 0.00 |
| ATOM | 1508 | HH12 | ARG | 1032 | 13.736 | 17.667 | 27.157 | 1.00 | 0.00 |
| ATOM | 1509 | NH2 | ARG | 1032 | 15.755 | 17.217 | 28.419 | 1.00 | 13.89 |
| ATOM | 1510 | HH21 | ARG | 1032 | 16.539 | 17.086 | 29.028 | 1.00 | 0.00 |
| ATOM | 1511 | HH22 | ARG | 1032 | 15.784 | 16.885 | 27.477 | 1.00 | 0.00 |
| ATOM | 1512 | C | ARG | 1032 | 15.782 | 22.956 | 32.172 | 1.00 | 11.51 |
| ATOM | 1513 | O | ARG | 1032 | 16.069 | 23.058 | 33.365 | 1.00 | 11.18 |
| ATOM | 1514 | N | ASN | 1033 | 16.744 | 22.998 | 31.211 | 1.00 | 10.80 |
| ATOM | 1515 | H | ASN | 1033 | 16.463 | 22.885 | 30.284 | 1.00 | 0.00 |
| ATOM | 1516 | CA | ASN | 1033 | 18.174 | 23.261 | 31.484 | 1.00 | 10.46 |
| ATOM | 1517 | CB | ASN | 1033 | 19.109 | 22.252 | 30.756 | 1.00 | 8.96 |
| ATOM | 1518 | CG | ASN | 1033 | 19.150 | 20.947 | 31.549 | 1.00 | 10.56 |
| ATOM | 1519 | OD1 | ASN | 1033 | 19.015 | 20.893 | 32.786 | 1.00 | 12.17 |
| ATOM | 1520 | ND2 | ASN | 1033 | 19.322 | 19.830 | 30.901 | 1.00 | 7.00 |
| ATOM | 1521 | HD21 | ASN | 1033 | 19.419 | 19.804 | 29.931 | 1.00 | 0.00 |
| ATOM | 1522 | HD22 | ASN | 1033 | 19.341 | 19.042 | 31.471 | 1.00 | 0.00 |
| ATOM | 1523 | C | ASN | 1033 | 18.669 | 24.670 | 31.160 | 1.00 | 9.24 |
| ATOM | 1524 | O | ASN | 1033 | 19.850 | 24.956 | 30.984 | 1.00 | 8.86 |
| ATOM | 1525 | N | ILE | 1034 | 17.695 | 25.585 | 31.140 | 1.00 | 10.69 |
| ATOM | 1526 | H | ILE | 1034 | 16.779 | 25.300 | 31.325 | 1.00 | 0.00 |
| ATOM | 1527 | CA | ILE | 1034 | 17.961 | 27.004 | 30.982 | 1.00 | 10.85 |
| ATOM | 1528 | CB | ILE | 1034 | 17.295 | 27.638 | 29.712 | 1.00 | 8.94 |
| ATOM | 1529 | CG2 | ILE | 1034 | 17.653 | 29.209 | 29.750 | 1.00 | 10.33 |
| ATOM | 1530 | CG1 | ILE | 1034 | 17.777 | 26.914 | 28.410 | 1.00 | 5.96 |
| ATOM | 1531 | CD1 | ILE | 1034 | 19.290 | 26.918 | 28.169 | 1.00 | 4.62 |
| ATOM | 1532 | C | ILE | 1034 | 17.414 | 27.726 | 32.226 | 1.00 | 9.32 |
| ATOM | 1533 | O | ILE | 1034 | 16.297 | 27.512 | 32.652 | 1.00 | 10.60 |
| ATOM | 1534 | N | LEU | 1035 | 18.201 | 28.563 | 32.847 | 1.00 | 8.65 |
| ATOM | 1535 | H | LEU | 1035 | 19.102 | 28.667 | 32.504 | 1.00 | 0.00 |
| ATOM | 1536 | CA | LEU | 1035 | 17.785 | 29.339 | 34.024 | 1.00 | 11.52 |
| ATOM | 1537 | CB | LEU | 1035 | 18.889 | 29.174 | 35.128 | 1.00 | 12.44 |
| ATOM | 1538 | CG | LEU | 1035 | 19.182 | 27.706 | 35.559 | 1.00 | 12.24 |
| ATOM | 1539 | CD1 | LEU | 1035 | 20.354 | 27.693 | 36.471 | 1.00 | 10.80 |
| ATOM | 1540 | CD2 | LEU | 1035 | 18.022 | 27.085 | 36.334 | 1.00 | 13.64 |
| ATOM | 1541 | C | LEU | 1035 | 17.534 | 30.831 | 33.770 | 1.00 | 10.83 |
| ATOM | 1542 | O | LEU | 1035 | 18.279 | 31.517 | 33.061 | 1.00 | 11.36 |
| ATOM | 1543 | N | LEU | 1036 | 16.541 | 31.395 | 34.410 | 1.00 | 9.61 |
| ATOM | 1544 | H | LEU | 1036 | 15.952 | 30.840 | 34.958 | 1.00 | 0.00 |
| ATOM | 1545 | CA | LEU | 1036 | 16.285 | 32.805 | 34.274 | 1.00 | 10.17 |
| ATOM | 1546 | CB | LEU | 1036 | 14.766 | 33.089 | 34.021 | 1.00 | 11.69 |
| ATOM | 1547 | CG | LEU | 1036 | 14.380 | 34.614 | 33.929 | 1.00 | 13.21 |
| ATOM | 1548 | CD1 | LEU | 1036 | 15.085 | 35.316 | 32.733 | 1.00 | 9.68 |
| ATOM | 1549 | CD2 | LEU | 1036 | 12.847 | 34.715 | 33.885 | 1.00 | 11.77 |
| ATOM | 1550 | C | LEU | 1036 | 16.717 | 33.497 | 35.579 | 1.00 | 12.75 |
| ATOM | 1551 | O | LEU | 1036 | 16.341 | 33.186 | 36.731 | 1.00 | 12.74 |
| ATOM | 1552 | N | SER | 1037 | 17.633 | 34.413 | 35.384 | 1.00 | 16.37 |
| ATOM | 1553 | H | SER | 1037 | 17.934 | 34.513 | 34.457 | 1.00 | 0.00 |
| ATOM | 1554 | CA | SER | 1037 | 18.219 | 35.266 | 36.442 | 1.00 | 20.46 |
| ATOM | 1555 | CB | SER | 1037 | 19.702 | 35.327 | 36.139 | 1.00 | 19.66 |
| ATOM | 1556 | OG | SER | 1037 | 20.325 | 35.431 | 37.377 | 1.00 | 26.65 |
| ATOM | 1557 | HG | SER | 1037 | 21.255 | 35.600 | 37.186 | 1.00 | 0.00 |
| ATOM | 1558 | C | SER | 1037 | 17.578 | 36.667 | 36.456 | 1.00 | 21.61 |
| ATOM | 1559 | O | SER | 1037 | 16.579 | 36.921 | 35.754 | 1.00 | 21.40 |
| ATOM | 1560 | N | GLU | 1038 | 18.079 | 37.644 | 37.232 | 1.00 | 23.42 |
| ATOM | 1561 | H | GLU | 1038 | 18.829 | 37.434 | 37.822 | 1.00 | 0.00 |
| ATOM | 1562 | CA | GLU | 1038 | 17.460 | 39.000 | 37.149 | 1.00 | 24.39 |
| ATOM | 1563 | CB | GLU | 1038 | 17.854 | 40.040 | 38.233 | 1.00 | 31.89 |
| ATOM | 1564 | CG | GLU | 1038 | 18.306 | 39.579 | 39.622 | 1.00 | 43.42 |
| ATOM | 1565 | CD | GLU | 1038 | 19.764 | 39.123 | 39.364 | 1.00 | 52.98 |
| ATOM | 1566 | OE1 | GLU | 1038 | 20.045 | 37.939 | 39.559 | 1.00 | 56.28 |
| ATOM | 1567 | OE2 | GLU | 1038 | 20.622 | 39.922 | 38.932 | 1.00 | 56.68 |
| ATOM | 1568 | C | GLU | 1038 | 17.880 | 39.706 | 35.869 | 1.00 | 22.37 |
| ATOM | 1569 | O | GLU | 1038 | 18.707 | 39.261 | 35.100 | 1.00 | 20.62 |
| ATOM | 1570 | N | LYS | 1039 | 17.267 | 40.804 | 35.558 | 1.00 | 22.33 |
| ATOM | 1571 | H | LYS | 1039 | 16.570 | 41.075 | 36.171 | 1.00 | 0.00 |
| ATOM | 1572 | CA | LYS | 1039 | 17.553 | 41.632 | 34.383 | 1.00 | 23.32 |
| ATOM | 1573 | CB | LYS | 1039 | 18.946 | 42.312 | 34.567 | 1.00 | 26.92 |
| ATOM | 1574 | CG | LYS | 1039 | 18.992 | 43.228 | 35.836 | 1.00 | 33.27 |
| ATOM | 1575 | CD | LYS | 1039 | 18.257 | 44.620 | 35.807 | 1.00 | 42.10 |
| ATOM | 1576 | CE | LYS | 1039 | 16.876 | 44.879 | 35.048 | 1.00 | 48.53 |
| ATOM | 1577 | NZ | LYS | 1039 | 17.021 | 45.062 | 33.562 | 1.00 | 52.83 |
| ATOM | 1578 | HZ1 | LYS | 1039 | 17.633 | 44.307 | 33.185 | 1.00 | 0.00 |
| ATOM | 1579 | HZ2 | LYS | 1039 | 17.447 | 45.983 | 33.332 | 1.00 | 0.00 |
| ATOM | 1580 | HZ3 | LYS | 1039 | 16.093 | 44.982 | 33.096 | 1.00 | 0.00 |
| ATOM | 1581 | C | LYS | 1039 | 17.486 | 40.912 | 33.092 | 1.00 | 20.89 |
| ATOM | 1582 | O | LYS | 1039 | 18.137 | 41.252 | 32.118 | 1.00 | 20.20 |
| ATOM | 1583 | N | ASN | 1040 | 16.573 | 39.967 | 33.045 | 1.00 | 21.23 |
| ATOM | 1584 | H | ASN | 1040 | 16.029 | 39.858 | 33.848 | 1.00 | 0.00 |
| ATOM | 1585 | CA | ASN | 1040 | 16.285 | 39.082 | 31.864 | 1.00 | 19.61 |
| ATOM | 1586 | CB | ASN | 1040 | 15.583 | 39.883 | 30.712 | 1.00 | 20.47 |
| ATOM | 1587 | CG | ASN | 1040 | 14.183 | 40.081 | 31.274 | 1.00 | 23.86 |
| ATOM | 1588 | OD1 | ASN | 1040 | 13.425 | 39.168 | 31.673 | 1.00 | 23.80 |
| ATOM | 1589 | ND2 | ASN | 1040 | 13.846 | 41.340 | 31.439 | 1.00 | 23.92 |
| ATOM | 1590 | HD21 | ASN | 1040 | 14.446 | 42.082 | 31.232 | 1.00 | 0.00 |
| ATOM | 1591 | HD22 | ASN | 1040 | 12.951 | 41.511 | 31.801 | 1.00 | 0.00 |
| ATOM | 1592 | C | ASN | 1040 | 17.439 | 38.325 | 31.270 | 1.00 | 17.19 |
| ATOM | 1593 | O | ASN | 1040 | 17.446 | 37.919 | 30.111 | 1.00 | 18.56 |
| ATOM | 1594 | N | VAL | 1041 | 18.402 | 38.045 | 32.138 | 1.00 | 14.89 |
| ATOM | 1595 | H | VAL | 1041 | 18.318 | 38.403 | 33.045 | 1.00 | 0.00 |
| ATOM | 1596 | CA | VAL | 1041 | 19.589 | 37.273 | 31.769 | 1.00 | 12.79 |
| ATOM | 1597 | CB | VAL | 1041 | 20.846 | 37.608 | 32.657 | 1.00 | 11.91 |
| ATOM | 1598 | CG1 | VAL | 1041 | 22.008 | 36.704 | 32.293 | 1.00 | 9.35 |
| ATOM | 1599 | CG2 | VAL | 1041 | 21.287 | 39.067 | 32.429 | 1.00 | 11.97 |
| ATOM | 1600 | C | VAL | 1041 | 19.236 | 35.814 | 31.975 | 1.00 | 13.01 |
| ATOM | 1601 | O | VAL | 1041 | 18.782 | 35.342 | 33.023 | 1.00 | 13.67 |
| ATOM | 1602 | N | VAL | 1042 | 19.463 | 35.080 | 30.910 | 1.00 | 13.97 |
| ATOM | 1603 | H | VAL | 1042 | 19.831 | 35.546 | 30.127 | 1.00 | 0.00 |
| ATOM | 1604 | CA | VAL | 1042 | 19.250 | 33.625 | 30.851 | 1.00 | 15.76 |
| ATOM | 1605 | CB | VAL | 1042 | 18.271 | 33.533 | 29.579 | 1.00 | 18.17 |
| ATOM | 1606 | CG1 | VAL | 1042 | 18.859 | 32.744 | 28.426 | 1.00 | 20.29 |
| ATOM | 1607 | CG2 | VAL | 1042 | 16.881 | 33.178 | 30.165 | 1.00 | 15.10 |
| ATOM | 1608 | C | VAL | 1042 | 20.642 | 32.904 | 30.842 | 1.00 | 14.66 |
| ATOM | 1609 | O | VAL | 1042 | 21.630 | 33.341 | 30.230 | 1.00 | 14.32 |
| ATOM | 1610 | N | LYS | 1043 | 20.734 | 31.807 | 31.588 | 1.00 | 12.86 |
| ATOM | 1611 | H | LYS | 1043 | 19.941 | 31.564 | 32.108 | 1.00 | 0.00 |
| ATOM | 1612 | CA | LYS | 1043 | 21.950 | 30.995 | 31.700 | 1.00 | 12.57 |
| ATOM | 1613 | CB | LYS | 1043 | 22.529 | 31.124 | 33.096 | 1.00 | 14.23 |
| ATOM | 1614 | CG | LYS | 1043 | 23.228 | 32.499 | 33.187 | 1.00 | 16.81 |
| ATOM | 1615 | CD | LYS | 1043 | 23.225 | 33.044 | 34.625 | 1.00 | 14.38 |
| ATOM | 1616 | CE | LYS | 1043 | 23.878 | 34.402 | 34.766 | 1.00 | 15.38 |
| ATOM | 1617 | NZ | LYS | 1043 | 25.305 | 34.219 | 34.469 | 1.00 | 17.26 |
| ATOM | 1618 | HZ1 | LYS | 1043 | 25.425 | 33.680 | 33.585 | 1.00 | 0.00 |
| ATOM | 1619 | HZ2 | LYS | 1043 | 25.761 | 33.703 | 35.241 | 1.00 | 0.00 |
| ATOM | 1620 | HZ3 | LYS | 1043 | 25.758 | 35.149 | 34.365 | 1.00 | 0.00 |
| ATOM | 1621 | C | LYS | 1043 | 21.751 | 29.500 | 31.420 | 1.00 | 10.92 |
| ATOM | 1622 | O | LYS | 1043 | 20.846 | 28.832 | 31.921 | 1.00 | 9.86 |
| ATOM | 1623 | N | ILE | 1044 | 22.647 | 28.931 | 30.618 | 1.00 | 11.73 |
| ATOM | 1624 | H | ILE | 1044 | 23.316 | 29.494 | 30.179 | 1.00 | 0.00 |
| ATOM | 1625 | CA | ILE | 1044 | 22.604 | 27.490 | 30.325 | 1.00 | 10.24 |
| ATOM | 1626 | CB | ILE | 1044 | 23.460 | 27.172 | 29.057 | 1.00 | 11.24 |
| ATOM | 1627 | CG2 | ILE | 1044 | 23.409 | 25.619 | 28.922 | 1.00 | 8.07 |
| ATOM | 1628 | CG1 | ILE | 1044 | 22.976 | 27.906 | 27.731 | 1.00 | 10.71 |
| ATOM | 1629 | CD1 | ILE | 1044 | 24.068 | 27.938 | 26.666 | 1.00 | 7.01 |
| ATOM | 1630 | C | ILE | 1044 | 23.201 | 26.817 | 31.585 | 1.00 | 11.59 |
| ATOM | 1631 | O | ILE | 1044 | 24.131 | 27.326 | 32.222 | 1.00 | 10.78 |
| ATOM | 1632 | N | CYS | 1045 | 22.666 | 25.648 | 31.933 | 1.00 | 11.82 |
| ATOM | 1633 | H | CYS | 1045 | 21.892 | 25.336 | 31.409 | 1.00 | 0.00 |
| ATOM | 1634 | CA | CYS | 1045 | 23.079 | 24.844 | 33.079 | 1.00 | 10.85 |
| ATOM | 1635 | CB | CYS | 1045 | 22.265 | 25.219 | 34.362 | 1.00 | 8.76 |
| ATOM | 1636 | SG | CYS | 1045 | 20.516 | 24.571 | 34.443 | 1.00 | 10.02 |
| ATOM | 1637 | C | CYS | 1045 | 22.736 | 23.394 | 32.761 | 1.00 | 11.51 |
| ATOM | 1638 | O | CYS | 1045 | 22.274 | 23.031 | 31.673 | 1.00 | 10.04 |
| ATOM | 1639 | N | ASP | 1046 | 23.050 | 22.495 | 33.652 | 1.00 | 8.84 |
| ATOM | 1640 | H | ASP | 1046 | 23.619 | 22.742 | 34.414 | 1.00 | 0.00 |
| ATOM | 1641 | CA | ASP | 1046 | 22.627 | 21.150 | 33.440 | 1.00 | 9.33 |
| ATOM | 1642 | CB | ASP | 1046 | 23.730 | 20.289 | 32.728 | 1.00 | 13.32 |
| ATOM | 1643 | CG | ASP | 1046 | 23.164 | 18.934 | 32.352 | 1.00 | 16.27 |
| ATOM | 1644 | OD1 | ASP | 1046 | 22.930 | 18.187 | 33.223 | 1.00 | 27.25 |
| ATOM | 1645 | OD2 | ASP | 1046 | 22.986 | 18.526 | 31.233 | 1.00 | 23.89 |
| ATOM | 1646 | C | ASP | 1046 | 22.355 | 20.599 | 34.832 | 1.00 | 11.27 |
| ATOM | 1647 | O | ASP | 1046 | 23.315 | 20.375 | 35.579 | 1.00 | 12.92 |
| ATOM | 1648 | N | PHE | 1047 | 21.088 | 20.398 | 35.219 | 1.00 | 9.88 |
| ATOM | 1649 | H | PHE | 1047 | 20.376 | 20.679 | 34.599 | 1.00 | 0.00 |
| ATOM | 1650 | CA | PHE | 1047 | 20.757 | 19.802 | 36.542 | 1.00 | 11.80 |
| ATOM | 1651 | CB | PHE | 1047 | 19.222 | 19.633 | 36.743 | 1.00 | 11.89 |
| ATOM | 1652 | CG | PHE | 1047 | 18.527 | 20.878 | 37.166 | 1.00 | 12.30 |
| ATOM | 1653 | CD1 | PHE | 1047 | 18.328 | 21.960 | 36.262 | 1.00 | 12.56 |
| ATOM | 1654 | CD2 | PHE | 1047 | 18.013 | 20.982 | 38.460 | 1.00 | 7.78 |
| ATOM | 1655 | CE1 | PHE | 1047 | 17.600 | 23.102 | 36.684 | 1.00 | 13.16 |
| ATOM | 1656 | CE2 | PHE | 1047 | 17.280 | 22.116 | 38.878 | 1.00 | 9.42 |
| ATOM | 1657 | CZ | PHE | 1047 | 17.059 | 23.173 | 38.000 | 1.00 | 12.87 |
| ATOM | 1658 | C | PHE | 1047 | 21.419 | 18.406 | 36.735 | 1.00 | 11.87 |
| ATOM | 1659 | O | PHE | 1047 | 21.755 | 17.978 | 37.843 | 1.00 | 11.69 |
| ATOM | 1660 | N | GLY | 1048 | 21.617 | 17.635 | 35.666 | 1.00 | 10.68 |
| ATOM | 1661 | H | GLY | 1048 | 21.269 | 17.975 | 34.826 | 1.00 | 0.00 |
| ATOM | 1662 | CA | GLY | 1048 | 22.252 | 16.299 | 35.701 | 1.00 | 11.46 |
| ATOM | 1663 | C | GLY | 1048 | 21.573 | 15.343 | 36.690 | 1.00 | 11.52 |
| ATOM | 1664 | O | GLY | 1048 | 20.351 | 15.069 | 36.667 | 1.00 | 14.19 |
| ATOM | 1665 | N | LEU | 1049 | 22.428 | 14.888 | 37.602 | 1.00 | 11.31 |
| ATOM | 1666 | H | LEU | 1049 | 23.358 | 15.079 | 37.378 | 1.00 | 0.00 |
| ATOM | 1667 | CA | LEU | 1049 | 22.141 | 13.978 | 38.726 | 1.00 | 12.93 |
| ATOM | 1668 | CB | LEU | 1049 | 23.433 | 13.667 | 39.477 | 1.00 | 14.88 |
| ATOM | 1669 | CG | LEU | 1049 | 24.181 | 12.324 | 39.297 | 1.00 | 19.55 |
| ATOM | 1670 | CD1 | LEU | 1049 | 23.773 | 11.555 | 38.036 | 1.00 | 21.51 |
| ATOM | 1671 | CD2 | LEU | 1049 | 25.645 | 12.649 | 39.412 | 1.00 | 21.23 |
| ATOM | 1672 | C | LEU | 1049 | 21.137 | 14.574 | 39.658 | 1.00 | 13.93 |
| ATOM | 1673 | O | LEU | 1049 | 20.499 | 13.838 | 40.406 | 1.00 | 13.22 |
| ATOM | 1674 | N | ALA | 1050 | 20.960 | 15.892 | 39.651 | 1.00 | 13.55 |
| ATOM | 1675 | H | ALA | 1050 | 21.497 | 16.446 | 39.052 | 1.00 | 0.00 |
| ATOM | 1676 | CA | ALA | 1050 | 19.931 | 16.551 | 40.504 | 1.00 | 14.90 |
| ATOM | 1677 | CB | ALA | 1050 | 20.288 | 18.016 | 40.872 | 1.00 | 13.55 |
| ATOM | 1678 | C | ALA | 1050 | 18.556 | 16.611 | 39.805 | 1.00 | 15.22 |
| ATOM | 1679 | O | ALA | 1050 | 17.702 | 17.466 | 40.064 | 1.00 | 16.00 |
| ATOM | 1680 | N | ARG | 1051 | 18.417 | 15.844 | 38.738 | 1.00 | 14.64 |
| ATOM | 1681 | H | ARG | 1051 | 19.172 | 15.324 | 38.390 | 1.00 | 0.00 |
| ATOM | 1682 | CA | ARG | 1051 | 17.127 | 15.759 | 38.073 | 1.00 | 17.53 |
| ATOM | 1683 | CB | ARG | 1051 | 17.212 | 16.077 | 36.572 | 1.00 | 16.36 |
| ATOM | 1684 | CG | ARG | 1051 | 15.778 | 15.911 | 36.037 | 1.00 | 17.81 |
| ATOM | 1685 | CD | ARG | 1051 | 15.712 | 16.421 | 34.631 | 1.00 | 16.00 |
| ATOM | 1686 | NE | ARG | 1051 | 15.637 | 17.874 | 34.682 | 1.00 | 16.07 |
| ATOM | 1687 | HE | ARG | 1051 | 14.851 | 18.299 | 35.084 | 1.00 | 0.00 |
| ATOM | 1688 | CZ | ARG | 1051 | 16.591 | 18.661 | 34.189 | 1.00 | 15.99 |
| ATOM | 1689 | NH1 | ARG | 1051 | 16.408 | 19.983 | 34.257 | 1.00 | 15.96 |
| ATOM | 1690 | HH11 | ARG | 1051 | 15.567 | 20.347 | 34.664 | 1.00 | 0.00 |
| ATOM | 1691 | HH12 | ARG | 1051 | 17.099 | 20.609 | 33.892 | 1.00 | 0.00 |
| ATOM | 1692 | NH2 | ARG | 1051 | 17.726 | 18.175 | 33.682 | 1.00 | 18.87 |
| ATOM | 1693 | HH21 | ARG | 1051 | 17.899 | 17.190 | 33.670 | 1.00 | 0.00 |
| ATOM | 1694 | HH22 | ARG | 1051 | 18.414 | 18.806 | 33.324 | 1.00 | 0.00 |
| ATOM | 1695 | C | ARG | 1051 | 16.715 | 14.307 | 38.200 | 1.00 | 17.98 |
| ATOM | 1696 | O | ARG | 1051 | 17.551 | 13.422 | 37.952 | 1.00 | 17.06 |
| ATOM | 1697 | N | ASP | 1052 | 15.502 | 14.036 | 38.614 | 1.00 | 18.96 |
| ATOM | 1698 | H | ASP | 1052 | 15.017 | 14.760 | 39.063 | 1.00 | 0.00 |
| ATOM | 1699 | CA | ASP | 1052 | 15.042 | 12.640 | 38.685 | 1.00 | 24.01 |
| ATOM | 1700 | CB | ASP | 1052 | 13.845 | 12.556 | 39.619 | 1.00 | 27.34 |
| ATOM | 1701 | CG | ASP | 1052 | 13.606 | 11.136 | 40.165 | 1.00 | 33.51 |
| ATOM | 1702 | OD1 | ASP | 1052 | 14.099 | 10.111 | 39.611 | 1.00 | 31.37 |
| ATOM | 1703 | OD2 | ASP | 1052 | 12.904 | 11.126 | 41.200 | 1.00 | 36.84 |
| ATOM | 1704 | C | ASP | 1052 | 14.653 | 12.118 | 37.288 | 1.00 | 25.02 |
| ATOM | 1705 | O | ASP | 1052 | 13.460 | 12.069 | 36.924 | 1.00 | 25.50 |
| ATOM | 1706 | N | ILE | 1053 | 15.621 | 11.683 | 36.492 | 1.00 | 24.60 |
| ATOM | 1707 | H | ILE | 1053 | 16.557 | 11.778 | 36.781 | 1.00 | 0.00 |
| ATOM | 1708 | CA | ILE | 1053 | 15.265 | 11.241 | 35.137 | 1.00 | 26.72 |
| ATOM | 1709 | CB | ILE | 1053 | 16.518 | 11.008 | 34.289 | 1.00 | 28.03 |
| ATOM | 1710 | CG2 | ILE | 1053 | 17.247 | 12.364 | 34.233 | 1.00 | 25.09 |
| ATOM | 1711 | CG1 | ILE | 1053 | 17.368 | 9.837 | 34.804 | 1.00 | 29.32 |
| ATOM | 1712 | CD1 | ILE | 1053 | 18.564 | 9.617 | 33.850 | 1.00 | 29.13 |
| ATOM | 1713 | C | ILE | 1053 | 14.388 | 10.001 | 35.060 | 1.00 | 29.05 |
| ATOM | 1714 | O | ILE | 1053 | 13.915 | 9.623 | 33.990 | 1.00 | 28.34 |
| ATOM | 1715 | N | TYR | 1054 | 14.247 | 9.302 | 36.182 | 1.00 | 29.71 |
| ATOM | 1716 | H | TYR | 1054 | 14.809 | 9.538 | 36.946 | 1.00 | 0.00 |
| ATOM | 1717 | CA | TYR | 1054 | 13.366 | 8.132 | 36.245 | 1.00 | 31.48 |
| ATOM | 1718 | CB | TYR | 1054 | 13.834 | 7.214 | 37.368 | 1.00 | 34.91 |
| ATOM | 1719 | C | TYR | 1054 | 11.917 | 8.582 | 36.495 | 1.00 | 31.67 |
| ATOM | 1720 | O | TYR | 1054 | 10.990 | 7.855 | 36.219 | 1.00 | 34.58 |
| ATOM | 1721 | N | LYS | 1055 | 11.654 | 9.700 | 37.150 | 1.00 | 32.28 |
| ATOM | 1722 | H | LYS | 1055 | 12.389 | 10.214 | 37.534 | 1.00 | 0.00 |
| ATOM | 1723 | CA | LYS | 1055 | 10.264 | 10.148 | 37.347 | 1.00 | 31.89 |
| ATOM | 1724 | CB | LYS | 1055 | 10.086 | 10.783 | 38.731 | 1.00 | 30.29 |
| ATOM | 1725 | C | LYS | 1055 | 9.886 | 11.208 | 36.278 | 1.00 | 32.88 |
| ATOM | 1726 | O | LYS | 1055 | 8.717 | 11.485 | 35.967 | 1.00 | 33.91 |
| ATOM | 1727 | N | ASP | 1056 | 10.893 | 11.892 | 35.718 | 1.00 | 31.58 |
| ATOM | 1728 | H | ASP | 1056 | 11.801 | 11.682 | 35.999 | 1.00 | 0.00 |
| ATOM | 1729 | CA | ASP | 1056 | 10.669 | 12.952 | 34.707 | 1.00 | 26.78 |
| ATOM | 1730 | CB | ASP | 1056 | 11.963 | 13.732 | 34.532 | 1.00 | 24.66 |
| ATOM | 1731 | CG | ASP | 1056 | 11.770 | 15.041 | 33.834 | 1.00 | 25.19 |
| ATOM | 1732 | OD1 | ASP | 1056 | 12.527 | 15.936 | 34.100 | 1.00 | 29.52 |
| ATOM | 1733 | OD2 | ASP | 1056 | 10.896 | 15.229 | 33.012 | 1.00 | 27.75 |
| ATOM | 1734 | C | ASP | 1056 | 10.172 | 12.498 | 33.344 | 1.00 | 25.34 |
| ATOM | 1735 | O | ASP | 1056 | 10.882 | 11.842 | 32.582 | 1.00 | 26.66 |
| ATOM | 1736 | N | PRO | 1057 | 8.992 | 12.967 | 32.928 | 1.00 | 24.66 |
| ATOM | 1737 | CD | PRO | 1057 | 8.067 | 13.795 | 33.695 | 1.00 | 23.67 |
| ATOM | 1738 | CA | PRO | 1057 | 8.422 | 12.584 | 31.638 | 1.00 | 24.43 |
| ATOM | 1739 | CB | PRO | 1057 | 7.051 | 13.275 | 31.668 | 1.00 | 24.45 |
| ATOM | 1740 | CG | PRO | 1057 | 7.298 | 14.449 | 32.594 | 1.00 | 24.63 |
| ATOM | 1741 | C | PRO | 1057 | 9.279 | 12.895 | 30.400 | 1.00 | 23.15 |
| ATOM | 1742 | O | PRO | 1057 | 9.163 | 12.233 | 29.370 | 1.00 | 23.87 |
| ATOM | 1743 | N | ASP | 1058 | 10.177 | 13.871 | 30.463 | 1.00 | 19.84 |
| ATOM | 1744 | H | ASP | 1058 | 10.273 | 14.330 | 31.316 | 1.00 | 0.00 |
| ATOM | 1745 | CA | ASP | 1058 | 11.052 | 14.243 | 29.304 | 1.00 | 19.06 |
| ATOM | 1746 | CB | ASP | 1058 | 11.714 | 15.592 | 29.636 | 1.00 | 17.76 |
| ATOM | 1747 | CG | ASP | 1058 | 10.716 | 16.731 | 29.862 | 1.00 | 20.26 |
| ATOM | 1748 | OD1 | ASP | 1058 | 11.147 | 17.797 | 30.304 | 1.00 | 19.57 |
| ATOM | 1749 | OD2 | ASP | 1058 | 9.524 | 16.568 | 29.547 | 1.00 | 20.95 |
| ATOM | 1750 | C | ASP | 1058 | 12.108 | 13.194 | 28.910 | 1.00 | 19.02 |
| ATOM | 1751 | O | ASP | 1058 | 12.747 | 13.255 | 27.835 | 1.00 | 20.42 |
| ATOM | 1752 | N | TYR | 1059 | 12.356 | 12.287 | 29.856 | 1.00 | 18.19 |
| ATOM | 1753 | H | TYR | 1059 | 11.824 | 12.282 | 30.675 | 1.00 | 0.00 |
| ATOM | 1754 | CA | TYR | 1059 | 13.320 | 11.224 | 29.657 | 1.00 | 19.80 |
| ATOM | 1755 | CB | TYR | 1059 | 14.244 | 11.095 | 30.919 | 1.00 | 17.13 |
| ATOM | 1756 | CG | TYR | 1059 | 14.920 | 12.382 | 31.069 | 1.00 | 14.44 |
| ATOM | 1757 | CD1 | TYR | 1059 | 14.288 | 13.433 | 31.734 | 1.00 | 14.82 |
| ATOM | 1758 | CE1 | TYR | 1059 | 14.963 | 14.653 | 31.802 | 1.00 | 15.36 |
| ATOM | 1759 | CD2 | TYR | 1059 | 16.187 | 12.545 | 30.487 | 1.00 | 16.12 |
| ATOM | 1760 | CE2 | TYR | 1059 | 16.870 | 13.765 | 30.556 | 1.00 | 13.72 |
| ATOM | 1761 | CZ | TYR | 1059 | 16.244 | 14.832 | 31.221 | 1.00 | 16.26 |
| ATOM | 1762 | OH | TYR | 1059 | 16.893 | 16.071 | 31.247 | 1.00 | 15.37 |
| ATOM | 1763 | HH | TYR | 1059 | 17.684 | 15.928 | 30.752 | 1.00 | 0.00 |
| ATOM | 1764 | C | TYR | 1059 | 12.537 | 9.909 | 29.363 | 1.00 | 23.58 |
| ATOM | 1765 | O | TYR | 1059 | 11.647 | 9.487 | 30.100 | 1.00 | 25.74 |
| ATOM | 1766 | N | VAL | 1060 | 12.840 | 9.268 | 28.240 | 1.00 | 23.40 |
| ATOM | 1767 | H | VAL | 1060 | 13.522 | 9.672 | 27.686 | 1.00 | 0.00 |
| ATOM | 1768 | CA | VAL | 1060 | 12.219 | 8.029 | 27.767 | 1.00 | 24.62 |
| ATOM | 1769 | CB | VAL | 1060 | 11.729 | 8.329 | 26.346 | 1.00 | 23.22 |
| ATOM | 1770 | CG1 | VAL | 1060 | 11.150 | 7.115 | 25.676 | 1.00 | 25.04 |
| ATOM | 1771 | CG2 | VAL | 1060 | 10.614 | 9.371 | 26.433 | 1.00 | 22.91 |
| ATOM | 1772 | C | VAL | 1060 | 13.202 | 6.884 | 27.822 | 1.00 | 24.55 |
| ATOM | 1773 | O | VAL | 1060 | 14.386 | 7.050 | 27.528 | 1.00 | 23.79 |
| ATOM | 1774 | N | ARG | 1061 | 12.771 | 5.718 | 28.329 | 1.00 | 28.17 |
| ATOM | 1775 | H | ARG | 1061 | 11.844 | 5.599 | 28.616 | 1.00 | 0.00 |
| ATOM | 1776 | CA | ARG | 1061 | 13.689 | 4.555 | 28.383 | 1.00 | 29.35 |
| ATOM | 1777 | CB | ARG | 1061 | 13.093 | 3.350 | 29.176 | 1.00 | 28.85 |
| ATOM | 1778 | C | ARG | 1061 | 14.029 | 4.046 | 26.966 | 1.00 | 31.49 |
| ATOM | 1779 | O | ARG | 1061 | 13.164 | 3.769 | 26.135 | 1.00 | 29.75 |
| ATOM | 1780 | N | LYS | 1062 | 15.307 | 4.080 | 26.641 | 1.00 | 33.46 |
| ATOM | 1781 | H | LYS | 1062 | 15.940 | 4.435 | 27.299 | 1.00 | 0.00 |
| ATOM | 1782 | CA | LYS | 1062 | 15.784 | 3.577 | 25.372 | 1.00 | 36.68 |
| ATOM | 1783 | CB | LYS | 1062 | 16.271 | 4.671 | 24.387 | 1.00 | 35.70 |
| ATOM | 1784 | C | LYS | 1062 | 16.992 | 2.767 | 25.847 | 1.00 | 39.49 |
| ATOM | 1785 | O | LYS | 1062 | 17.898 | 3.295 | 26.503 | 1.00 | 40.19 |
| ATOM | 1786 | N | GLY | 1063 | 16.976 | 1.445 | 25.611 | 1.00 | 42.19 |
| ATOM | 1787 | H | GLY | 1063 | 16.176 | 1.070 | 25.194 | 1.00 | 0.00 |
| ATOM | 1788 | CA | GLY | 1063 | 18.066 | 0.544 | 26.028 | 1.00 | 44.40 |
| ATOM | 1789 | C | GLY | 1063 | 18.212 | 0.641 | 27.539 | 1.00 | 45.83 |
| ATOM | 1790 | O | GLY | 1063 | 17.194 | 0.649 | 28.247 | 1.00 | 46.41 |
| ATOM | 1791 | N | ASP | 1064 | 19.452 | 0.682 | 28.059 | 1.00 | 47.75 |
| ATOM | 1792 | H | ASP | 1064 | 20.232 | 0.618 | 27.467 | 1.00 | 0.00 |
| ATOM | 1793 | CA | ASP | 1064 | 19.615 | 0.822 | 29.542 | 1.00 | 49.27 |
| ATOM | 1794 | CB | ASP | 1064 | 20.915 | 0.041 | 30.046 | 1.00 | 53.99 |
| ATOM | 1795 | CG | ASP | 1064 | 20.684 | −1.488 | 30.178 | 1.00 | 58.07 |
| ATOM | 1796 | OD1 | ASP | 1064 | 19.665 | −1.910 | 30.758 | 1.00 | 60.86 |
| ATOM | 1797 | OD2 | ASP | 1064 | 21.539 | −2.249 | 29.702 | 1.00 | 59.76 |
| ATOM | 1798 | C | ASP | 1064 | 19.667 | 2.315 | 30.011 | 1.00 | 46.74 |
| ATOM | 1799 | O | ASP | 1064 | 19.975 | 2.656 | 31.160 | 1.00 | 46.23 |
| ATOM | 1800 | N | ALA | 1065 | 19.359 | 3.242 | 29.096 | 1.00 | 42.79 |
| ATOM | 1801 | H | ALA | 1065 | 19.073 | 2.944 | 28.210 | 1.00 | 0.00 |
| ATOM | 1802 | CA | ALA | 1065 | 19.345 | 4.679 | 29.389 | 1.00 | 37.95 |
| ATOM | 1803 | CB | ALA | 1065 | 20.216 | 5.358 | 28.359 | 1.00 | 36.02 |
| ATOM | 1804 | C | ALA | 1065 | 17.947 | 5.316 | 29.353 | 1.00 | 35.92 |
| ATOM | 1805 | O | ALA | 1065 | 17.027 | 4.820 | 28.716 | 1.00 | 36.36 |
| ATOM | 1806 | N | ARG | 1066 | 17.726 | 6.381 | 30.106 | 1.00 | 31.64 |
| ATOM | 1807 | H | ARG | 1066 | 18.449 | 6.680 | 30.689 | 1.00 | 0.00 |
| ATOM | 1808 | CA | ARG | 1066 | 16.453 | 7.134 | 30.055 | 1.00 | 29.92 |
| ATOM | 1809 | CB | ARG | 1066 | 15.848 | 7.293 | 31.485 | 1.00 | 27.88 |
| ATOM | 1810 | C | ARG | 1066 | 16.915 | 8.492 | 29.417 | 1.00 | 27.53 |
| ATOM | 1811 | O | ARG | 1066 | 17.651 | 9.311 | 29.989 | 1.00 | 24.43 |
| ATOM | 1812 | N | LEU | 1067 | 16.657 | 8.582 | 28.105 | 1.00 | 25.33 |
| ATOM | 1813 | H | LEU | 1067 | 16.135 | 7.847 | 27.705 | 1.00 | 0.00 |
| ATOM | 1814 | CA | LEU | 1067 | 17.072 | 9.712 | 27.261 | 1.00 | 22.13 |
| ATOM | 1815 | CB | LEU | 1067 | 17.524 | 9.085 | 25.942 | 1.00 | 22.52 |
| ATOM | 1816 | CG | LEU | 1067 | 18.637 | 8.044 | 26.100 | 1.00 | 24.34 |
| ATOM | 1817 | CD1 | LEU | 1067 | 18.648 | 7.194 | 24.846 | 1.00 | 25.35 |
| ATOM | 1818 | CD2 | LEU | 1067 | 20.015 | 8.682 | 26.273 | 1.00 | 25.54 |
| ATOM | 1819 | C | LEU | 1067 | 16.085 | 10.834 | 27.022 | 1.00 | 19.74 |
| ATOM | 1820 | O | LEU | 1067 | 14.886 | 10.576 | 26.953 | 1.00 | 20.57 |
| ATOM | 1821 | N | PRO | 1068 | 16.514 | 12.095 | 26.829 | 1.00 | 16.83 |
| ATOM | 1822 | CD | PRO | 1068 | 17.894 | 12.594 | 26.980 | 1.00 | 13.07 |
| ATOM | 1823 | CA | PRO | 1068 | 15.584 | 13.207 | 26.436 | 1.00 | 17.55 |
| ATOM | 1824 | CB | PRO | 1068 | 16.340 | 14.473 | 26.812 | 1.00 | 12.43 |
| ATOM | 1825 | CG | PRO | 1068 | 17.746 | 14.015 | 26.417 | 1.00 | 12.72 |
| ATOM | 1826 | C | PRO | 1068 | 15.246 | 13.118 | 24.908 | 1.00 | 17.39 |
| ATOM | 1827 | O | PRO | 1068 | 15.586 | 13.981 | 24.070 | 1.00 | 15.68 |
| ATOM | 1828 | N | LEU | 1069 | 14.511 | 12.065 | 24.547 | 1.00 | 17.62 |
| ATOM | 1829 | H | LEU | 1069 | 14.205 | 11.453 | 25.251 | 1.00 | 0.00 |
| ATOM | 1830 | CA | LEU | 1069 | 14.171 | 11.784 | 23.144 | 1.00 | 18.32 |
| ATOM | 1831 | CB | LEU | 1069 | 13.290 | 10.489 | 23.138 | 1.00 | 22.67 |
| ATOM | 1832 | CG | LEU | 1069 | 13.919 | 9.025 | 22.959 | 1.00 | 26.88 |
| ATOM | 1833 | CD1 | LEU | 1069 | 15.251 | 9.024 | 22.133 | 1.00 | 26.83 |
| ATOM | 1834 | CD2 | LEU | 1069 | 14.369 | 8.474 | 24.275 | 1.00 | 30.33 |
| ATOM | 1835 | C | LEU | 1069 | 13.510 | 12.906 | 22.305 | 1.00 | 17.35 |
| ATOM | 1836 | O | LEU | 1069 | 13.855 | 13.119 | 21.143 | 1.00 | 16.55 |
| ATOM | 1837 | N | LYS | 1070 | 12.559 | 13.682 | 22.805 | 1.00 | 16.29 |
| ATOM | 1838 | H | LYS | 1070 | 12.141 | 13.442 | 23.652 | 1.00 | 0.00 |
| ATOM | 1839 | CA | LYS | 1070 | 11.948 | 14.773 | 22.023 | 1.00 | 15.31 |
| ATOM | 1840 | CB | LYS | 1070 | 10.772 | 15.341 | 22.831 | 1.00 | 12.67 |
| ATOM | 1841 | CG | LYS | 1070 | 9.649 | 14.336 | 22.945 | 1.00 | 15.20 |
| ATOM | 1842 | CD | LYS | 1070 | 8.310 | 14.945 | 23.352 | 1.00 | 13.69 |
| ATOM | 1843 | CE | LYS | 1070 | 8.386 | 15.451 | 24.748 | 1.00 | 18.32 |
| ATOM | 1844 | NZ | LYS | 1070 | 7.015 | 15.810 | 25.146 | 1.00 | 18.86 |
| ATOM | 1845 | HZ1 | LYS | 1070 | 6.402 | 15.017 | 24.878 | 1.00 | 0.00 |
| ATOM | 1846 | HZ2 | LYS | 1070 | 6.702 | 16.660 | 24.637 | 1.00 | 0.00 |
| ATOM | 1847 | HZ3 | LYS | 1070 | 6.945 | 15.973 | 26.170 | 1.00 | 0.00 |
| ATOM | 1848 | C | LYS | 1070 | 12.911 | 15.921 | 21.616 | 1.00 | 15.10 |
| ATOM | 1849 | O | LYS | 1070 | 12.562 | 16.791 | 20.816 | 1.00 | 14.34 |
| ATOM | 1850 | N | TRP | 1071 | 14.095 | 15.974 | 22.269 | 1.00 | 14.23 |
| ATOM | 1851 | H | TRP | 1071 | 14.275 | 15.289 | 22.942 | 1.00 | 0.00 |
| ATOM | 1852 | CA | TRP | 1071 | 15.126 | 17.001 | 22.040 | 1.00 | 13.76 |
| ATOM | 1853 | CB | TRP | 1071 | 15.644 | 17.517 | 23.387 | 1.00 | 9.42 |
| ATOM | 1854 | CG | TRP | 1071 | 14.717 | 18.384 | 24.135 | 1.00 | 10.36 |
| ATOM | 1855 | CD2 | TRP | 1071 | 13.589 | 18.024 | 24.923 | 1.00 | 11.17 |
| ATOM | 1856 | CE2 | TRP | 1071 | 12.953 | 19.242 | 25.294 | 1.00 | 12.63 |
| ATOM | 1857 | CE3 | TRP | 1071 | 13.015 | 16.784 | 25.324 | 1.00 | 12.10 |
| ATOM | 1858 | CD1 | TRP | 1071 | 14.739 | 19.778 | 24.109 | 1.00 | 9.84 |
| ATOM | 1859 | NE1 | TRP | 1071 | 13.667 | 20.276 | 24.764 | 1.00 | 10.51 |
| ATOM | 1860 | HE1 | TRP | 1071 | 13.439 | 21.211 | 24.776 | 1.00 | 0.00 |
| ATOM | 1861 | CZ2 | TRP | 1071 | 11.754 | 19.192 | 26.047 | 1.00 | 12.27 |
| ATOM | 1862 | CZ3 | TRP | 1071 | 11.839 | 16.763 | 26.098 | 1.00 | 13.68 |
| ATOM | 1863 | CH2 | TRP | 1071 | 11.203 | 17.963 | 26.451 | 1.00 | 10.07 |
| ATOM | 1864 | C | TRP | 1071 | 16.309 | 16.470 | 21.240 | 1.00 | 14.54 |
| ATOM | 1865 | O | TRP | 1071 | 17.233 | 17.195 | 20.863 | 1.00 | 14.21 |
| ATOM | 1866 | N | MET | 1072 | 16.309 | 15.162 | 21.006 | 1.00 | 14.72 |
| ATOM | 1867 | H | MET | 1072 | 15.539 | 14.625 | 21.288 | 1.00 | 0.00 |
| ATOM | 1868 | CA | MET | 1072 | 17.416 | 14.549 | 20.302 | 1.00 | 15.92 |
| ATOM | 1869 | CB | MET | 1072 | 17.586 | 13.154 | 20.845 | 1.00 | 17.46 |
| ATOM | 1870 | CG | MET | 1072 | 18.170 | 13.146 | 22.268 | 1.00 | 18.40 |
| ATOM | 1871 | SD | MET | 1072 | 17.908 | 11.533 | 23.094 | 1.00 | 20.58 |
| ATOM | 1872 | CE | MET | 1072 | 19.156 | 10.567 | 22.210 | 1.00 | 16.27 |
| ATOM | 1873 | C | MET | 1072 | 17.401 | 14.497 | 18.796 | 1.00 | 16.71 |
| ATOM | 1874 | O | MET | 1072 | 16.414 | 14.062 | 18.188 | 1.00 | 19.44 |
| ATOM | 1875 | N | ALA | 1073 | 18.520 | 14.832 | 18.173 | 1.00 | 16.04 |
| ATOM | 1876 | H | ALA | 1073 | 19.202 | 15.228 | 18.746 | 1.00 | 0.00 |
| ATOM | 1877 | CA | ALA | 1073 | 18.623 | 14.752 | 16.697 | 1.00 | 18.51 |
| ATOM | 1878 | CB | ALA | 1073 | 19.985 | 15.311 | 16.273 | 1.00 | 16.05 |
| ATOM | 1879 | C | ALA | 1073 | 18.466 | 13.292 | 16.167 | 1.00 | 20.79 |
| ATOM | 1880 | O | ALA | 1073 | 18.859 | 12.328 | 16.867 | 1.00 | 19.42 |
| ATOM | 1881 | N | PRO | 1074 | 18.026 | 13.046 | 14.902 | 1.00 | 23.07 |
| ATOM | 1882 | CD | PRO | 1074 | 17.685 | 14.021 | 13.842 | 1.00 | 22.37 |
| ATOM | 1883 | CA | PRO | 1074 | 17.947 | 11.624 | 14.365 | 1.00 | 23.46 |
| ATOM | 1884 | CB | PRO | 1074 | 17.511 | 11.760 | 12.880 | 1.00 | 23.59 |
| ATOM | 1885 | CG | PRO | 1074 | 16.869 | 13.158 | 12.825 | 1.00 | 24.69 |
| ATOM | 1886 | C | PRO | 1074 | 19.282 | 10.843 | 14.506 | 1.00 | 23.31 |
| ATOM | 1887 | O | PRO | 1074 | 19.316 | 9.717 | 15.002 | 1.00 | 22.30 |
| ATOM | 1888 | N | GLU | 1075 | 20.446 | 11.432 | 14.182 | 1.00 | 22.55 |
| ATOM | 1889 | H | GLU | 1075 | 20.392 | 12.353 | 13.866 | 1.00 | 0.00 |
| ATOM | 1890 | CA | GLU | 1075 | 21.718 | 10.673 | 14.327 | 1.00 | 22.16 |
| ATOM | 1891 | CB | GLU | 1075 | 22.919 | 11.438 | 13.753 | 1.00 | 20.97 |
| ATOM | 1892 | CG | GLU | 1075 | 23.418 | 12.787 | 14.357 | 1.00 | 21.73 |
| ATOM | 1893 | CD | GLU | 1075 | 22.552 | 14.019 | 14.108 | 1.00 | 22.70 |
| ATOM | 1894 | OE1 | GLU | 1075 | 21.472 | 13.916 | 13.485 | 1.00 | 18.63 |
| ATOM | 1895 | OE2 | GLU | 1075 | 22.993 | 15.087 | 14.565 | 1.00 | 20.61 |
| ATOM | 1896 | C | GLU | 1075 | 22.031 | 10.308 | 15.755 | 1.00 | 22.72 |
| ATOM | 1897 | O | GLU | 1075 | 22.732 | 9.340 | 15.995 | 1.00 | 23.22 |
| ATOM | 1898 | N | THR | 1076 | 21.485 | 11.012 | 16.758 | 1.00 | 21.66 |
| ATOM | 1899 | H | THR | 1076 | 20.894 | 11.762 | 16.542 | 1.00 | 0.00 |
| ATOM | 1900 | CA | THR | 1076 | 21.753 | 10.674 | 18.189 | 1.00 | 22.13 |
| ATOM | 1901 | CB | THR | 1076 | 21.512 | 11.855 | 19.195 | 1.00 | 20.77 |
| ATOM | 1902 | OG1 | THR | 1076 | 22.162 | 13.069 | 18.759 | 1.00 | 21.70 |
| ATOM | 1903 | HG1 | THR | 1076 | 22.350 | 13.610 | 19.537 | 1.00 | 0.00 |
| ATOM | 1904 | CG2 | THR | 1076 | 22.048 | 11.458 | 20.554 | 1.00 | 19.94 |
| ATOM | 1905 | C | THR | 1076 | 20.840 | 9.546 | 18.614 | 1.00 | 21.24 |
| ATOM | 1906 | O | THR | 1076 | 21.273 | 8.640 | 19.307 | 1.00 | 22.21 |
| ATOM | 1907 | N | ILE | 1077 | 19.588 | 9.588 | 18.216 | 1.00 | 20.24 |
| ATOM | 1908 | H | ILE | 1077 | 19.305 | 10.359 | 17.679 | 1.00 | 0.00 |
| ATOM | 1909 | CA | ILE | 1077 | 18.617 | 8.554 | 18.527 | 1.00 | 22.51 |
| ATOM | 1910 | CB | ILE | 1077 | 17.235 | 8.994 | 17.942 | 1.00 | 23.18 |
| ATOM | 1911 | CG2 | ILE | 1077 | 16.210 | 7.818 | 17.948 | 1.00 | 21.76 |
| ATOM | 1912 | CG1 | ILE | 1077 | 16.686 | 10.198 | 18.759 | 1.00 | 20.69 |
| ATOM | 1913 | CD1 | ILE | 1077 | 15.355 | 10.645 | 18.089 | 1.00 | 20.25 |
| ATOM | 1914 | C | ILE | 1077 | 19.114 | 7.246 | 17.865 | 1.00 | 23.82 |
| ATOM | 1915 | O | ILE | 1077 | 19.303 | 6.215 | 18.488 | 1.00 | 24.80 |
| ATOM | 1916 | N | PHE | 1078 | 19.393 | 7.303 | 16.593 | 1.00 | 24.53 |
| ATOM | 1917 | H | PHE | 1078 | 19.278 | 8.154 | 16.131 | 1.00 | 0.00 |
| ATOM | 1918 | CA | PHE | 1078 | 19.809 | 6.121 | 15.835 | 1.00 | 25.71 |
| ATOM | 1919 | CB | PHE | 1078 | 19.401 | 6.362 | 14.355 | 1.00 | 28.19 |
| ATOM | 1920 | CG | PHE | 1078 | 17.924 | 6.517 | 14.225 | 1.00 | 28.83 |
| ATOM | 1921 | CD1 | PHE | 1078 | 17.387 | 7.763 | 13.838 | 1.00 | 30.54 |
| ATOM | 1922 | CD2 | PHE | 1078 | 17.065 | 5.433 | 14.517 | 1.00 | 30.28 |
| ATOM | 1923 | CE1 | PHE | 1078 | 16.005 | 7.945 | 13.749 | 1.00 | 27.83 |
| ATOM | 1924 | CE2 | PHE | 1078 | 15.683 | 5.618 | 14.423 | 1.00 | 29.63 |
| ATOM | 1925 | CZ | PHE | 1078 | 15.183 | 6.873 | 14.047 | 1.00 | 28.29 |
| ATOM | 1926 | C | PHE | 1078 | 21.257 | 5.697 | 15.918 | 1.00 | 26.20 |
| ATOM | 1927 | O | PHE | 1078 | 21.558 | 4.512 | 15.999 | 1.00 | 28.34 |
| ATOM | 1928 | N | ASP | 1079 | 22.193 | 6.613 | 15.834 | 1.00 | 25.31 |
| ATOM | 1929 | H | ASP | 1079 | 21.927 | 7.541 | 15.678 | 1.00 | 0.00 |
| ATOM | 1930 | CA | ASP | 1079 | 23.634 | 6.243 | 15.922 | 1.00 | 27.48 |
| ATOM | 1931 | CB | ASP | 1079 | 24.369 | 6.868 | 14.720 | 1.00 | 29.18 |
| ATOM | 1932 | CG | ASP | 1079 | 23.606 | 6.525 | 13.441 | 1.00 | 35.25 |
| ATOM | 1933 | OD1 | ASP | 1079 | 23.009 | 5.443 | 13.356 | 1.00 | 35.56 |
| ATOM | 1934 | OD2 | ASP | 1079 | 23.587 | 7.361 | 12.527 | 1.00 | 39.44 |
| ATOM | 1935 | C | ASP | 1079 | 24.374 | 6.617 | 17.211 | 1.00 | 26.16 |
| ATOM | 1936 | O | ASP | 1079 | 25.579 | 6.445 | 17.319 | 1.00 | 24.78 |
| ATOM | 1937 | N | ARG | 1080 | 23.681 | 7.136 | 18.227 | 1.00 | 26.25 |
| ATOM | 1938 | H | ARG | 1080 | 22.711 | 7.214 | 18.128 | 1.00 | 0.00 |
| ATOM | 1939 | CA | ARG | 1080 | 24.296 | 7.556 | 19.507 | 1.00 | 27.00 |
| ATOM | 1940 | CB | ARG | 1080 | 24.801 | 6.296 | 20.282 | 1.00 | 29.85 |
| ATOM | 1941 | CG | ARG | 1080 | 23.568 | 5.447 | 20.655 | 1.00 | 36.91 |
| ATOM | 1942 | CD | ARG | 1080 | 23.870 | 4.155 | 21.419 | 1.00 | 43.98 |
| ATOM | 1943 | NE | ARG | 1080 | 24.830 | 3.325 | 20.656 | 1.00 | 52.39 |
| ATOM | 1944 | HE | ARG | 1080 | 25.355 | 3.730 | 19.934 | 1.00 | 0.00 |
| ATOM | 1945 | CZ | ARG | 1080 | 25.051 | 2.041 | 20.997 | 1.00 | 55.08 |
| ATOM | 1946 | NH1 | ARG | 1080 | 25.926 | 1.327 | 20.288 | 1.00 | 55.49 |
| ATOM | 1947 | HH11 | ARG | 1080 | 26.402 | 1.739 | 19.516 | 1.00 | 0.00 |
| ATOM | 1948 | HH12 | ARG | 1080 | 26.112 | 0.372 | 20.522 | 1.00 | 0.00 |
| ATOM | 1949 | NH2 | ARG | 1080 | 24.379 | 1.447 | 22.007 | 1.00 | 57.11 |
| ATOM | 1950 | HH21 | ARG | 1080 | 23.699 | 1.948 | 22.539 | 1.00 | 0.00 |
| ATOM | 1951 | HH22 | ARG | 1080 | 24.580 | 0.491 | 22.224 | 1.00 | 0.00 |
| ATOM | 1952 | C | ARG | 1080 | 25.421 | 8.531 | 19.259 | 1.00 | 24.84 |
| ATOM | 1953 | O | ARG | 1080 | 26.414 | 8.637 | 19.969 | 1.00 | 25.51 |
| ATOM | 1954 | N | VAL | 1081 | 25.287 | 9.300 | 18.209 | 1.00 | 23.54 |
| ATOM | 1955 | H | VAL | 1081 | 24.502 | 9.194 | 17.640 | 1.00 | 0.00 |
| ATOM | 1956 | CA | VAL | 1081 | 26.308 | 10.281 | 17.873 | 1.00 | 21.42 |
| ATOM | 1957 | CB | VAL | 1081 | 26.372 | 10.418 | 16.339 | 1.00 | 22.00 |
| ATOM | 1958 | CG1 | VAL | 1081 | 27.223 | 11.609 | 15.924 | 1.00 | 21.89 |
| ATOM | 1959 | CG2 | VAL | 1081 | 27.089 | 9.219 | 15.768 | 1.00 | 21.70 |
| ATOM | 1960 | C | VAL | 1081 | 25.967 | 11.602 | 18.511 | 1.00 | 20.77 |
| ATOM | 1961 | O | VAL | 1081 | 24.853 | 12.083 | 18.287 | 1.00 | 21.11 |
| ATOM | 1962 | N | TYR | 1082 | 26.836 | 12.184 | 19.347 | 1.00 | 18.96 |
| ATOM | 1963 | H | TYR | 1082 | 27.640 | 11.680 | 19.574 | 1.00 | 0.00 |
| ATOM | 1964 | CA | TYR | 1082 | 26.591 | 13.525 | 19.944 | 1.00 | 18.10 |
| ATOM | 1965 | CB | TYR | 1082 | 26.728 | 13.547 | 21.489 | 1.00 | 18.98 |
| ATOM | 1966 | CG | TYR | 1082 | 25.718 | 12.780 | 22.211 | 1.00 | 20.13 |
| ATOM | 1967 | CD1 | TYR | 1082 | 25.860 | 11.387 | 22.356 | 1.00 | 22.82 |
| ATOM | 1968 | CE1 | TYR | 1082 | 24.837 | 10.636 | 22.998 | 1.00 | 24.04 |
| ATOM | 1969 | CD2 | TYR | 1082 | 24.582 | 13.437 | 22.695 | 1.00 | 19.88 |
| ATOM | 1970 | CE2 | TYR | 1082 | 23.583 | 12.684 | 23.330 | 1.00 | 21.27 |
| ATOM | 1971 | CZ | TYR | 1082 | 23.691 | 11.297 | 23.472 | 1.00 | 21.80 |
| ATOM | 1972 | OH | TYR | 1082 | 22.633 | 10.570 | 23.980 | 1.00 | 23.40 |
| ATOM | 1973 | HH | TYR | 1082 | 22.893 | 9.645 | 24.028 | 1.00 | 0.00 |
| ATOM | 1974 | C | TYR | 1082 | 27.614 | 14.552 | 19.399 | 1.00 | 19.84 |
| ATOM | 1975 | O | TYR | 1082 | 28.824 | 14.323 | 19.418 | 1.00 | 19.93 |
| ATOM | 1976 | N | THR | 1083 | 27.193 | 15.675 | 18.797 | 1.00 | 18.33 |
| ATOM | 1977 | H | THR | 1083 | 26.228 | 15.832 | 18.750 | 1.00 | 0.00 |
| ATOM | 1978 | CA | THR | 1083 | 28.121 | 16.711 | 18.270 | 1.00 | 17.28 |
| ATOM | 1979 | CB | THR | 1083 | 28.367 | 16.524 | 16.730 | 1.00 | 17.78 |
| ATOM | 1980 | OG1 | THR | 1083 | 27.208 | 16.974 | 16.034 | 1.00 | 19.19 |
| ATOM | 1981 | HG1 | THR | 1083 | 27.367 | 16.780 | 15.090 | 1.00 | 0.00 |
| ATOM | 1982 | CG2 | THR | 1083 | 28.725 | 15.051 | 16.370 | 1.00 | 17.63 |
| ATOM | 1983 | C | THR | 1083 | 27.531 | 18.139 | 18.543 | 1.00 | 15.31 |
| ATOM | 1984 | O | THR | 1083 | 26.390 | 18.158 | 18.975 | 1.00 | 14.90 |
| ATOM | 1985 | N | ILE | 1084 | 28.177 | 19.349 | 18.425 | 1.00 | 15.29 |
| ATOM | 1986 | H | ILE | 1084 | 29.151 | 19.367 | 18.275 | 1.00 | 0.00 |
| ATOM | 1987 | CA | ILE | 1084 | 27.393 | 20.605 | 18.745 | 1.00 | 15.51 |
| ATOM | 1988 | CB | ILE | 1084 | 28.074 | 22.072 | 18.689 | 1.00 | 18.91 |
| ATOM | 1989 | CG2 | ILE | 1084 | 28.055 | 22.834 | 20.102 | 1.00 | 17.08 |
| ATOM | 1990 | CG1 | ILE | 1084 | 29.375 | 21.868 | 17.966 | 1.00 | 22.06 |
| ATOM | 1991 | CD1 | ILE | 1084 | 29.295 | 21.705 | 16.431 | 1.00 | 16.92 |
| ATOM | 1992 | C | ILE | 1084 | 26.321 | 20.681 | 17.703 | 1.00 | 13.12 |
| ATOM | 1993 | O | ILE | 1084 | 25.359 | 21.370 | 17.917 | 1.00 | 11.48 |
| ATOM | 1994 | N | GLN | 1085 | 26.459 | 20.001 | 16.548 | 1.00 | 15.29 |
| ATOM | 1995 | H | GLN | 1085 | 27.305 | 19.538 | 16.380 | 1.00 | 0.00 |
| ATOM | 1996 | CA | GLN | 1085 | 25.365 | 20.044 | 15.534 | 1.00 | 17.41 |
| ATOM | 1997 | CB | GLN | 1085 | 25.770 | 19.496 | 14.092 | 1.00 | 17.64 |
| ATOM | 1998 | CG | GLN | 1085 | 26.671 | 20.592 | 13.376 | 1.00 | 18.83 |
| ATOM | 1999 | CD | GLN | 1085 | 26.236 | 22.094 | 13.497 | 1.00 | 21.22 |
| ATOM | 2000 | OE1 | GLN | 1085 | 26.756 | 22.924 | 14.264 | 1.00 | 22.33 |
| ATOM | 2001 | NE2 | GLN | 1085 | 25.277 | 22.540 | 12.722 | 1.00 | 20.51 |
| ATOM | 2002 | HE21 | GLN | 1085 | 24.818 | 21.961 | 12.082 | 1.00 | 0.00 |
| ATOM | 2003 | HE22 | GLN | 1085 | 25.012 | 23.478 | 12.805 | 1.00 | 0.00 |
| ATOM | 2004 | C | GLN | 1085 | 24.167 | 19.289 | 16.037 | 1.00 | 15.85 |
| ATOM | 2005 | O | GLN | 1085 | 23.039 | 19.574 | 15.653 | 1.00 | 15.45 |
| ATOM | 2006 | N | SER | 1086 | 24.329 | 18.264 | 16.847 | 1.00 | 15.04 |
| ATOM | 2007 | H | SER | 1086 | 25.216 | 17.946 | 17.114 | 1.00 | 0.00 |
| ATOM | 2008 | CA | SER | 1086 | 23.115 | 17.645 | 17.394 | 1.00 | 14.79 |
| ATOM | 2009 | CB | SER | 1086 | 23.388 | 16.209 | 17.891 | 1.00 | 14.65 |
| ATOM | 2010 | OG | SER | 1086 | 24.299 | 16.110 | 18.923 | 1.00 | 13.69 |
| ATOM | 2011 | HG | SER | 1086 | 24.297 | 15.198 | 19.239 | 1.00 | 0.00 |
| ATOM | 2012 | C | SER | 1086 | 22.619 | 18.595 | 18.520 | 1.00 | 15.96 |
| ATOM | 2013 | O | SER | 1086 | 21.415 | 18.684 | 18.810 | 1.00 | 19.76 |
| ATOM | 2014 | N | ASP | 1087 | 23.496 | 19.393 | 19.171 | 1.00 | 16.08 |
| ATOM | 2015 | H | ASP | 1087 | 24.455 | 19.270 | 19.010 | 1.00 | 0.00 |
| ATOM | 2016 | CA | ASP | 1087 | 23.000 | 20.409 | 20.170 | 1.00 | 15.55 |
| ATOM | 2017 | CB | ASP | 1087 | 24.137 | 21.178 | 20.942 | 1.00 | 14.72 |
| ATOM | 2018 | CG | ASP | 1087 | 24.792 | 20.342 | 22.047 | 1.00 | 14.26 |
| ATOM | 2019 | OD1 | ASP | 1087 | 25.847 | 20.761 | 22.502 | 1.00 | 14.92 |
| ATOM | 2020 | OD2 | ASP | 1087 | 24.271 | 19.330 | 22.517 | 1.00 | 15.51 |
| ATOM | 2021 | C | ASP | 1087 | 22.198 | 21.500 | 19.395 | 1.00 | 16.25 |
| ATOM | 2022 | O | ASP | 1087 | 21.302 | 22.101 | 19.990 | 1.00 | 15.61 |
| ATOM | 2023 | N | VAL | 1088 | 22.542 | 21.832 | 18.094 | 1.00 | 14.64 |
| ATOM | 2024 | H | VAL | 1088 | 23.390 | 21.501 | 17.749 | 1.00 | 0.00 |
| ATOM | 2025 | CA | VAL | 1088 | 21.761 | 22.810 | 17.307 | 1.00 | 12.31 |
| ATOM | 2026 | CB | VAL | 1088 | 22.395 | 23.087 | 15.919 | 1.00 | 13.18 |
| ATOM | 2027 | CG1 | VAL | 1088 | 21.397 | 23.836 | 14.986 | 1.00 | 9.31 |
| ATOM | 2028 | CG2 | VAL | 1088 | 23.604 | 24.036 | 16.122 | 1.00 | 10.69 |
| ATOM | 2029 | C | VAL | 1088 | 20.378 | 22.231 | 17.125 | 1.00 | 12.72 |
| ATOM | 2030 | O | VAL | 1088 | 19.407 | 22.968 | 17.333 | 1.00 | 14.83 |
| ATOM | 2031 | N | TRP | 1089 | 20.235 | 20.923 | 16.825 | 1.00 | 11.68 |
| ATOM | 2032 | H | TRP | 1089 | 21.041 | 20.398 | 16.619 | 1.00 | 0.00 |
| ATOM | 2033 | CA | TRP | 1089 | 18.906 | 20.301 | 16.741 | 1.00 | 11.09 |
| ATOM | 2034 | CB | TRP | 1089 | 19.017 | 18.774 | 16.540 | 1.00 | 13.31 |
| ATOM | 2035 | CG | TRP | 1089 | 17.662 | 18.121 | 16.470 | 1.00 | 14.82 |
| ATOM | 2036 | CD2 | TRP | 1089 | 16.974 | 17.650 | 15.299 | 1.00 | 14.04 |
| ATOM | 2037 | CE2 | TRP | 1089 | 15.705 | 17.187 | 15.717 | 1.00 | 14.01 |
| ATOM | 2038 | CE3 | TRP | 1089 | 17.328 | 17.569 | 13.931 | 1.00 | 15.02 |
| ATOM | 2039 | CD1 | TRP | 1089 | 16.767 | 17.932 | 17.540 | 1.00 | 13.39 |
| ATOM | 2040 | NE1 | TRP | 1089 | 15.601 | 17.362 | 17.086 | 1.00 | 13.52 |
| ATOM | 2041 | HE1 | TRP | 1089 | 14.821 | 17.083 | 17.632 | 1.00 | 0.00 |
| ATOM | 2042 | CZ2 | TRP | 1089 | 14.803 | 16.670 | 14.756 | 1.00 | 14.67 |
| ATOM | 2043 | CZ3 | TRP | 1089 | 16.429 | 17.037 | 12.982 | 1.00 | 14.57 |
| ATOM | 2044 | CH2 | TRP | 1089 | 15.171 | 16.589 | 13.401 | 1.00 | 16.41 |
| ATOM | 2045 | C | TRP | 1089 | 18.110 | 20.564 | 18.050 | 1.00 | 12.77 |
| ATOM | 2046 | O | TRP | 1089 | 16.977 | 21.069 | 18.027 | 1.00 | 12.13 |
| ATOM | 2047 | N | SER | 1090 | 18.709 | 20.225 | 19.230 | 1.00 | 14.14 |
| ATOM | 2048 | H | SER | 1090 | 19.549 | 19.716 | 19.212 | 1.00 | 0.00 |
| ATOM | 2049 | CA | SER | 1090 | 18.054 | 20.470 | 20.565 | 1.00 | 13.41 |
| ATOM | 2050 | CB | SER | 1090 | 18.923 | 20.060 | 21.786 | 1.00 | 12.03 |
| ATOM | 2051 | OG | SER | 1090 | 19.302 | 18.707 | 21.576 | 1.00 | 12.15 |
| ATOM | 2052 | HG | SER | 1090 | 19.503 | 18.265 | 22.409 | 1.00 | 0.00 |
| ATOM | 2053 | C | SER | 1090 | 17.773 | 21.949 | 20.718 | 1.00 | 12.77 |
| ATOM | 2054 | O | SER | 1090 | 16.745 | 22.313 | 21.292 | 1.00 | 12.00 |
| ATOM | 2055 | N | PHE | 1091 | 18.659 | 22.854 | 20.223 | 1.00 | 12.28 |
| ATOM | 2056 | H | PHE | 1091 | 19.465 | 22.510 | 19.783 | 1.00 | 0.00 |
| ATOM | 2057 | CA | PHE | 1091 | 18.398 | 24.325 | 20.302 | 1.00 | 11.24 |
| ATOM | 2058 | CB | PHE | 1091 | 19.593 | 25.102 | 19.746 | 1.00 | 10.19 |
| ATOM | 2059 | CG | PHE | 1091 | 19.366 | 26.553 | 19.860 | 1.00 | 10.96 |
| ATOM | 2060 | CD1 | PHE | 1091 | 19.565 | 27.209 | 21.095 | 1.00 | 10.49 |
| ATOM | 2061 | CD2 | PHE | 1091 | 18.996 | 27.292 | 18.716 | 1.00 | 11.67 |
| ATOM | 2062 | CE1 | PHE | 1091 | 19.410 | 28.604 | 21.211 | 1.00 | 5.78 |
| ATOM | 2063 | CE2 | PHE | 1091 | 18.836 | 28.684 | 18.834 | 1.00 | 11.29 |
| ATOM | 2064 | CZ | PHE | 1091 | 19.050 | 29.328 | 20.081 | 1.00 | 10.28 |
| ATOM | 2065 | C | PHE | 1091 | 17.147 | 24.628 | 19.480 | 1.00 | 12.65 |
| ATOM | 2066 | O | PHE | 1091 | 16.337 | 25.431 | 19.927 | 1.00 | 13.66 |
| ATOM | 2067 | N | GLY | 1092 | 16.907 | 24.018 | 18.289 | 1.00 | 12.25 |
| ATOM | 2068 | H | GLY | 1092 | 17.599 | 23.430 | 17.919 | 1.00 | 0.00 |
| ATOM | 2069 | CA | GLY | 1092 | 15.647 | 24.277 | 17.532 | 1.00 | 8.94 |
| ATOM | 2070 | C | GLY | 1092 | 14.447 | 23.836 | 18.396 | 1.00 | 9.76 |
| ATOM | 2071 | O | GLY | 1092 | 13.402 | 24.460 | 18.425 | 1.00 | 9.81 |
| ATOM | 2072 | N | VAL | 1093 | 14.515 | 22.728 | 19.142 | 1.00 | 8.43 |
| ATOM | 2073 | H | VAL | 1093 | 15.338 | 22.211 | 19.080 | 1.00 | 0.00 |
| ATOM | 2074 | CA | VAL | 1093 | 13.413 | 22.278 | 20.019 | 1.00 | 9.72 |
| ATOM | 2075 | CB | VAL | 1093 | 13.687 | 20.831 | 20.600 | 1.00 | 11.43 |
| ATOM | 2076 | CG1 | VAL | 1093 | 12.479 | 20.419 | 21.511 | 1.00 | 11.16 |
| ATOM | 2077 | CG2 | VAL | 1093 | 13.891 | 19.813 | 19.454 | 1.00 | 9.81 |
| ATOM | 2078 | C | VAL | 1093 | 13.264 | 23.285 | 21.163 | 1.00 | 9.28 |
| ATOM | 2079 | O | VAL | 1093 | 12.153 | 23.612 | 21.551 | 1.00 | 9.97 |
| ATOM | 2080 | N | LEU | 1094 | 14.350 | 23.820 | 21.756 | 1.00 | 10.99 |
| ATOM | 2081 | H | LEU | 1094 | 15.216 | 23.494 | 21.438 | 1.00 | 0.00 |
| ATOM | 2082 | CA | LEU | 1094 | 14.302 | 24.850 | 22.808 | 1.00 | 11.18 |
| ATOM | 2083 | CB | LEU | 1094 | 15.718 | 25.270 | 23.170 | 1.00 | 10.45 |
| ATOM | 2084 | CG | LEU | 1094 | 16.043 | 25.960 | 24.520 | 1.00 | 17.69 |
| ATOM | 2085 | CD1 | LEU | 1094 | 16.927 | 27.067 | 24.060 | 1.00 | 14.34 |
| ATOM | 2086 | CD2 | LEU | 1094 | 14.901 | 26.542 | 25.385 | 1.00 | 16.32 |
| ATOM | 2087 | C | LEU | 1094 | 13.542 | 26.092 | 22.261 | 1.00 | 10.70 |
| ATOM | 2088 | O | LEU | 1094 | 12.690 | 26.653 | 22.961 | 1.00 | 10.69 |
| ATOM | 2089 | N | LEU | 1095 | 13.844 | 26.550 | 21.024 | 1.00 | 11.07 |
| ATOM | 2090 | H | LEU | 1095 | 14.612 | 26.142 | 20.568 | 1.00 | 0.00 |
| ATOM | 2091 | CA | LEU | 1095 | 13.139 | 27.667 | 20.390 | 1.00 | 12.09 |
| ATOM | 2092 | CB | LEU | 1095 | 13.555 | 27.898 | 18.937 | 1.00 | 10.35 |
| ATOM | 2093 | CG | LEU | 1095 | 14.976 | 28.409 | 18.782 | 1.00 | 14.19 |
| ATOM | 2094 | CD1 | LEU | 1095 | 15.276 | 28.524 | 17.284 | 1.00 | 14.00 |
| ATOM | 2095 | CD2 | LEU | 1095 | 15.170 | 29.752 | 19.501 | 1.00 | 11.15 |
| ATOM | 2096 | C | LEU | 1095 | 11.649 | 27.368 | 20.359 | 1.00 | 11.89 |
| ATOM | 2097 | O | LEU | 1095 | 10.822 | 28.228 | 20.675 | 1.00 | 12.46 |
| ATOM | 2098 | N | TRP | 1096 | 11.249 | 26.136 | 20.004 | 1.00 | 12.06 |
| ATOM | 2099 | H | TRP | 1096 | 11.922 | 25.475 | 19.735 | 1.00 | 0.00 |
| ATOM | 2100 | CA | TRP | 1096 | 9.811 | 25.781 | 19.983 | 1.00 | 11.39 |
| ATOM | 2101 | CB | TRP | 1096 | 9.644 | 24.334 | 19.413 | 1.00 | 10.42 |
| ATOM | 2102 | CG | TRP | 1096 | 8.206 | 23.958 | 19.183 | 1.00 | 11.06 |
| ATOM | 2103 | CD2 | TRP | 1096 | 7.293 | 23.494 | 20.165 | 1.00 | 11.17 |
| ATOM | 2104 | CE2 | TRP | 1096 | 6.014 | 23.350 | 19.497 | 1.00 | 12.85 |
| ATOM | 2105 | CE3 | TRP | 1096 | 7.446 | 23.166 | 21.547 | 1.00 | 10.95 |
| ATOM | 2106 | CD1 | TRP | 1096 | 7.451 | 24.053 | 17.960 | 1.00 | 10.79 |
| ATOM | 2107 | NE1 | TRP | 1096 | 6.145 | 23.697 | 18.152 | 1.00 | 10.37 |
| ATOM | 2108 | HE1 | TRP | 1096 | 5.475 | 23.694 | 17.430 | 1.00 | 0.00 |
| ATOM | 2109 | CZ2 | TRP | 1096 | 4.930 | 22.872 | 20.276 | 1.00 | 14.26 |
| ATOM | 2110 | CZ3 | TRP | 1096 | 6.351 | 22.740 | 22.282 | 1.00 | 10.09 |
| ATOM | 2111 | CH2 | TRP | 1096 | 5.093 | 22.596 | 21.659 | 1.00 | 12.72 |
| ATOM | 2112 | C | TRP | 1096 | 9.259 | 25.893 | 21.412 | 1.00 | 12.97 |
| ATOM | 2113 | O | TRP | 1096 | 8.157 | 26.420 | 21.626 | 1.00 | 12.73 |
| ATOM | 2114 | N | GLU | 1097 | 10.026 | 25.428 | 22.428 | 1.00 | 11.49 |
| ATOM | 2115 | H | GLU | 1097 | 10.872 | 24.984 | 22.217 | 1.00 | 0.00 |
| ATOM | 2116 | CA | GLU | 1097 | 9.559 | 25.567 | 23.818 | 1.00 | 10.93 |
| ATOM | 2117 | CB | GLU | 1097 | 10.526 | 24.928 | 24.875 | 1.00 | 11.19 |
| ATOM | 2118 | CG | GLU | 1097 | 10.633 | 23.443 | 24.681 | 1.00 | 11.79 |
| ATOM | 2119 | CD | GLU | 1097 | 11.621 | 22.931 | 25.735 | 1.00 | 14.75 |
| ATOM | 2120 | OE1 | GLU | 1097 | 11.163 | 22.552 | 26.821 | 1.00 | 14.16 |
| ATOM | 2121 | OE2 | GLU | 1097 | 12.825 | 22.920 | 25.463 | 1.00 | 11.18 |
| ATOM | 2122 | C | GLU | 1097 | 9.435 | 27.038 | 24.197 | 1.00 | 10.76 |
| ATOM | 2123 | O | GLU | 1097 | 8.506 | 27.410 | 24.902 | 1.00 | 7.86 |
| ATOM | 2124 | N | ILE | 1098 | 10.353 | 27.907 | 23.750 | 1.00 | 9.23 |
| ATOM | 2125 | H | ILE | 1098 | 11.097 | 27.577 | 23.213 | 1.00 | 0.00 |
| ATOM | 2126 | CA | ILE | 1098 | 10.252 | 29.308 | 24.094 | 1.00 | 9.97 |
| ATOM | 2127 | CB | ILE | 1098 | 11.534 | 30.070 | 23.707 | 1.00 | 9.65 |
| ATOM | 2128 | CG2 | ILE | 1098 | 11.296 | 31.598 | 23.822 | 1.00 | 10.81 |
| ATOM | 2129 | CG1 | ILE | 1098 | 12.686 | 29.665 | 24.650 | 1.00 | 9.86 |
| ATOM | 2130 | CD1 | ILE | 1098 | 14.037 | 30.272 | 24.152 | 1.00 | 8.28 |
| ATOM | 2131 | C | ILE | 1098 | 9.070 | 29.934 | 23.418 | 1.00 | 12.80 |
| ATOM | 2132 | O | ILE | 1098 | 8.259 | 30.590 | 24.095 | 1.00 | 11.90 |
| ATOM | 2133 | N | PHE | 1099 | 8.920 | 29.742 | 22.104 | 1.00 | 12.73 |
| ATOM | 2134 | H | PHE | 1099 | 9.598 | 29.209 | 21.639 | 1.00 | 0.00 |
| ATOM | 2135 | CA | PHE | 1099 | 7.809 | 30.354 | 21.394 | 1.00 | 12.90 |
| ATOM | 2136 | CB | PHE | 1099 | 8.174 | 30.545 | 19.934 | 1.00 | 13.94 |
| ATOM | 2137 | CG | PHE | 1099 | 9.233 | 31.575 | 19.939 | 1.00 | 14.10 |
| ATOM | 2138 | CD1 | PHE | 1099 | 10.606 | 31.219 | 20.094 | 1.00 | 12.81 |
| ATOM | 2139 | CD2 | PHE | 1099 | 8.866 | 32.932 | 19.781 | 1.00 | 16.55 |
| ATOM | 2140 | CE1 | PHE | 1099 | 11.603 | 32.210 | 20.124 | 1.00 | 13.19 |
| ATOM | 2141 | CE2 | PHE | 1099 | 9.872 | 33.926 | 19.801 | 1.00 | 14.99 |
| ATOM | 2142 | CZ | PHE | 1099 | 11.246 | 33.567 | 19.974 | 1.00 | 15.28 |
| ATOM | 2143 | C | PHE | 1099 | 6.467 | 29.664 | 21.538 | 1.00 | 13.75 |
| ATOM | 2144 | O | PHE | 1099 | 5.487 | 30.025 | 20.885 | 1.00 | 12.75 |
| ATOM | 2145 | N | SER | 1100 | 6.391 | 28.645 | 22.373 | 1.00 | 12.62 |
| ATOM | 2146 | H | SER | 1100 | 7.227 | 28.264 | 22.736 | 1.00 | 0.00 |
| ATOM | 2147 | CA | SER | 1100 | 5.115 | 27.971 | 22.742 | 1.00 | 13.99 |
| ATOM | 2148 | CB | SER | 1100 | 5.176 | 26.428 | 22.720 | 1.00 | 15.12 |
| ATOM | 2149 | OG | SER | 1100 | 6.075 | 25.929 | 23.740 | 1.00 | 14.11 |
| ATOM | 2150 | HG | SER | 1100 | 6.151 | 24.975 | 23.640 | 1.00 | 0.00 |
| ATOM | 2151 | C | SER | 1100 | 4.825 | 28.342 | 24.208 | 1.00 | 14.39 |
| ATOM | 2152 | O | SER | 1100 | 3.809 | 27.961 | 24.793 | 1.00 | 13.50 |
| ATOM | 2153 | N | LEU | 1101 | 5.676 | 29.213 | 24.807 | 1.00 | 14.61 |
| ATOM | 2154 | H | LEU | 1101 | 6.342 | 29.645 | 24.242 | 1.00 | 0.00 |
| ATOM | 2155 | CA | LEU | 1101 | 5.661 | 29.619 | 26.232 | 1.00 | 12.80 |
| ATOM | 2156 | CB | LEU | 1101 | 4.493 | 30.566 | 26.581 | 1.00 | 11.12 |
| ATOM | 2157 | CG | LEU | 1101 | 4.693 | 31.977 | 26.024 | 1.00 | 12.01 |
| ATOM | 2158 | CD1 | LEU | 1101 | 3.442 | 32.786 | 26.490 | 1.00 | 13.55 |
| ATOM | 2159 | CD2 | LEU | 1101 | 6.045 | 32.630 | 26.498 | 1.00 | 10.11 |
| ATOM | 2160 | C | LEU | 1101 | 5.603 | 28.423 | 27.197 | 1.00 | 12.17 |
| ATOM | 2161 | O | LEU | 1101 | 4.754 | 28.178 | 28.030 | 1.00 | 11.67 |
| ATOM | 2162 | N | GLY | 1102 | 6.602 | 27.592 | 27.054 | 1.00 | 13.34 |
| ATOM | 2163 | H | GLY | 1102 | 7.228 | 27.755 | 26.324 | 1.00 | 0.00 |
| ATOM | 2164 | CA | GLY | 1102 | 6.820 | 26.428 | 27.917 | 1.00 | 14.92 |
| ATOM | 2165 | C | GLY | 1102 | 6.013 | 25.202 | 27.657 | 1.00 | 14.24 |
| ATOM | 2166 | O | GLY | 1102 | 5.810 | 24.373 | 28.546 | 1.00 | 11.85 |
| ATOM | 2167 | N | ALA | 1103 | 5.506 | 25.067 | 26.456 | 1.00 | 14.14 |
| ATOM | 2168 | H | ALA | 1103 | 5.652 | 25.769 | 25.788 | 1.00 | 0.00 |
| ATOM | 2169 | CA | ALA | 1103 | 4.726 | 23.846 | 26.176 | 1.00 | 14.65 |
| ATOM | 2170 | CB | ALA | 1103 | 3.836 | 23.980 | 24.885 | 1.00 | 12.07 |
| ATOM | 2171 | C | ALA | 1103 | 5.635 | 22.609 | 25.995 | 1.00 | 15.57 |
| ATOM | 2172 | O | ALA | 1103 | 6.839 | 22.679 | 25.749 | 1.00 | 14.22 |
| ATOM | 2173 | N | SER | 1104 | 5.054 | 21.408 | 26.118 | 1.00 | 15.92 |
| ATOM | 2174 | H | SER | 1104 | 4.091 | 21.410 | 26.273 | 1.00 | 0.00 |
| ATOM | 2175 | CA | SER | 1104 | 5.821 | 20.151 | 25.928 | 1.00 | 16.84 |
| ATOM | 2176 | CB | SER | 1104 | 5.097 | 19.000 | 26.670 | 1.00 | 16.32 |
| ATOM | 2177 | OG | SER | 1104 | 5.789 | 17.779 | 26.487 | 1.00 | 19.58 |
| ATOM | 2178 | HG | SER | 1104 | 5.335 | 17.084 | 26.978 | 1.00 | 0.00 |
| ATOM | 2179 | C | SER | 1104 | 5.931 | 19.837 | 24.423 | 1.00 | 14.24 |
| ATOM | 2180 | O | SER | 1104 | 4.902 | 19.805 | 23.732 | 1.00 | 13.86 |
| ATOM | 2181 | N | PRO | 1105 | 7.135 | 19.605 | 23.895 | 1.00 | 13.01 |
| ATOM | 2182 | CD | PRO | 1105 | 8.447 | 19.673 | 24.575 | 1.00 | 10.32 |
| ATOM | 2183 | CA | PRO | 1105 | 7.279 | 19.286 | 22.489 | 1.00 | 13.27 |
| ATOM | 2184 | CB | PRO | 1105 | 8.791 | 19.030 | 22.362 | 1.00 | 10.09 |
| ATOM | 2185 | CG | PRO | 1105 | 9.354 | 19.988 | 23.400 | 1.00 | 9.47 |
| ATOM | 2186 | C | PRO | 1105 | 6.376 | 18.112 | 22.010 | 1.00 | 16.99 |
| ATOM | 2187 | O | PRO | 1105 | 5.923 | 17.238 | 22.763 | 1.00 | 17.21 |
| ATOM | 2188 | N | TYR | 1106 | 6.183 | 18.037 | 20.691 | 1.00 | 18.28 |
| ATOM | 2189 | H | TYR | 1106 | 6.572 | 18.758 | 20.161 | 1.00 | 0.00 |
| ATOM | 2190 | CA | TYR | 1106 | 5.360 | 16.997 | 19.992 | 1.00 | 18.30 |
| ATOM | 2191 | CB | TYR | 1106 | 6.177 | 15.703 | 19.924 | 1.00 | 15.36 |
| ATOM | 2192 | CG | TYR | 1106 | 7.395 | 15.970 | 19.146 | 1.00 | 15.64 |
| ATOM | 2193 | CD1 | TYR | 1106 | 8.616 | 16.261 | 19.787 | 1.00 | 13.80 |
| ATOM | 2194 | CE1 | TYR | 1106 | 9.748 | 16.540 | 19.014 | 1.00 | 16.07 |
| ATOM | 2195 | CD2 | TYR | 1106 | 7.338 | 15.903 | 17.745 | 1.00 | 15.68 |
| ATOM | 2196 | CE2 | TYR | 1106 | 8.484 | 16.195 | 16.979 | 1.00 | 15.32 |
| ATOM | 2197 | CZ | TYR | 1106 | 9.700 | 16.489 | 17.599 | 1.00 | 14.65 |
| ATOM | 2198 | OH | TYR | 1106 | 10.822 | 16.769 | 16.820 | 1.00 | 14.04 |
| ATOM | 2199 | HH | TYR | 1106 | 10.572 | 16.749 | 15.887 | 1.00 | 0.00 |
| ATOM | 2200 | C | TYR | 1106 | 4.023 | 16.776 | 20.693 | 1.00 | 19.15 |
| ATOM | 2201 | O | TYR | 1106 | 3.778 | 15.691 | 21.220 | 1.00 | 19.05 |
| ATOM | 2202 | N | PRO | 1107 | 3.170 | 17.842 | 20.806 | 1.00 | 19.33 |
| ATOM | 2203 | CD | PRO | 1107 | 3.342 | 19.260 | 20.347 | 1.00 | 18.88 |
| ATOM | 2204 | CA | PRO | 1107 | 1.877 | 17.700 | 21.469 | 1.00 | 20.66 |
| ATOM | 2205 | CB | PRO | 1107 | 1.158 | 19.013 | 21.199 | 1.00 | 19.22 |
| ATOM | 2206 | CG | PRO | 1107 | 1.924 | 19.723 | 20.088 | 1.00 | 18.99 |
| ATOM | 2207 | C | PRO | 1107 | 1.075 | 16.498 | 20.968 | 1.00 | 24.32 |
| ATOM | 2208 | O | PRO | 1107 | 0.818 | 16.349 | 19.769 | 1.00 | 22.35 |
| ATOM | 2209 | N | GLY | 1108 | 0.765 | 15.607 | 21.914 | 1.00 | 28.77 |
| ATOM | 2210 | H | GLY | 1108 | 1.129 | 15.799 | 22.805 | 1.00 | 0.00 |
| ATOM | 2211 | CA | GLY | 1108 | −0.024 | 14.360 | 21.693 | 1.00 | 33.25 |
| ATOM | 2212 | C | GLY | 1108 | 0.672 | 13.097 | 21.104 | 1.00 | 36.31 |
| ATOM | 2213 | O | GLY | 1108 | 0.130 | 11.986 | 21.158 | 1.00 | 39.74 |
| ATOM | 2214 | N | VAL | 1109 | 1.851 | 13.239 | 20.474 | 1.00 | 36.02 |
| ATOM | 2215 | H | VAL | 1109 | 2.260 | 14.132 | 20.519 | 1.00 | 0.00 |
| ATOM | 2216 | CA | VAL | 1109 | 2.588 | 12.123 | 19.880 | 1.00 | 32.35 |
| ATOM | 2217 | CB | VAL | 1109 | 3.693 | 12.732 | 18.985 | 1.00 | 32.87 |
| ATOM | 2218 | CG1 | VAL | 1109 | 4.233 | 11.666 | 18.065 | 1.00 | 35.32 |
| ATOM | 2219 | CG2 | VAL | 1109 | 3.160 | 13.828 | 18.042 | 1.00 | 33.33 |
| ATOM | 2220 | C | VAL | 1109 | 3.175 | 11.205 | 20.968 | 1.00 | 32.29 |
| ATOM | 2221 | O | VAL | 1109 | 3.744 | 11.635 | 21.974 | 1.00 | 32.53 |
| ATOM | 2222 | N | LYS | 1110 | 2.950 | 9.898 | 20.894 | 1.00 | 31.68 |
| ATOM | 2223 | H | LYS | 1110 | 2.341 | 9.563 | 20.205 | 1.00 | 0.00 |
| ATOM | 2224 | CA | LYS | 1110 | 3.591 | 8.997 | 21.877 | 1.00 | 30.41 |
| ATOM | 2225 | CB | LYS | 1110 | 2.917 | 7.576 | 21.918 | 1.00 | 31.54 |
| ATOM | 2226 | C | LYS | 1110 | 5.041 | 8.869 | 21.380 | 1.00 | 28.64 |
| ATOM | 2227 | O | LYS | 1110 | 5.356 | 8.802 | 20.192 | 1.00 | 28.75 |
| ATOM | 2228 | N | ILE | 1111 | 5.996 | 8.921 | 22.254 | 1.00 | 28.70 |
| ATOM | 2229 | H | ILE | 1111 | 5.768 | 9.129 | 23.174 | 1.00 | 0.00 |
| ATOM | 2230 | CA | ILE | 1111 | 7.395 | 8.828 | 21.846 | 1.00 | 29.16 |
| ATOM | 2231 | CB | ILE | 1111 | 8.250 | 9.717 | 22.838 | 1.00 | 24.49 |
| ATOM | 2232 | CG2 | ILE | 1111 | 9.775 | 9.686 | 22.534 | 1.00 | 21.75 |
| ATOM | 2233 | CG1 | ILE | 1111 | 7.646 | 11.155 | 22.753 | 1.00 | 20.94 |
| ATOM | 2234 | CD1 | ILE | 1111 | 7.380 | 11.824 | 21.387 | 1.00 | 17.72 |
| ATOM | 2235 | C | ILE | 1111 | 7.760 | 7.363 | 21.805 | 1.00 | 31.83 |
| ATOM | 2236 | O | ILE | 1111 | 7.996 | 6.677 | 22.813 | 1.00 | 34.10 |
| ATOM | 2237 | N | ASP | 1112 | 7.706 | 6.894 | 20.576 | 1.00 | 31.82 |
| ATOM | 2238 | H | ASP | 1112 | 7.451 | 7.520 | 19.872 | 1.00 | 0.00 |
| ATOM | 2239 | CA | ASP | 1112 | 7.996 | 5.494 | 20.285 | 1.00 | 33.27 |
| ATOM | 2240 | CB | ASP | 1112 | 6.652 | 4.710 | 20.243 | 1.00 | 33.97 |
| ATOM | 2241 | CG | ASP | 1112 | 5.739 | 5.076 | 19.078 | 1.00 | 34.59 |
| ATOM | 2242 | OD1 | ASP | 1112 | 4.574 | 4.705 | 19.153 | 1.00 | 38.39 |
| ATOM | 2243 | OD2 | ASP | 1112 | 6.151 | 5.700 | 18.110 | 1.00 | 32.54 |
| ATOM | 2244 | C | ASP | 1112 | 8.758 | 5.277 | 18.994 | 1.00 | 34.44 |
| ATOM | 2245 | O | ASP | 1112 | 9.287 | 6.214 | 18.406 | 1.00 | 34.30 |
| ATOM | 2246 | N | GLU | 1113 | 8.749 | 4.035 | 18.482 | 1.00 | 35.64 |
| ATOM | 2247 | H | GLU | 1113 | 8.339 | 3.325 | 19.012 | 1.00 | 0.00 |
| ATOM | 2248 | CA | GLU | 1113 | 9.443 | 3.674 | 17.244 | 1.00 | 34.77 |
| ATOM | 2249 | CB | GLU | 1113 | 9.370 | 2.120 | 17.041 | 1.00 | 38.24 |
| ATOM | 2250 | C | GLU | 1113 | 8.829 | 4.414 | 16.068 | 1.00 | 35.32 |
| ATOM | 2251 | O | GLU | 1113 | 9.524 | 4.924 | 15.184 | 1.00 | 36.50 |
| ATOM | 2252 | N | GLU | 1114 | 7.514 | 4.520 | 16.020 | 1.00 | 34.70 |
| ATOM | 2253 | H | GLU | 1114 | 6.971 | 4.058 | 16.683 | 1.00 | 0.00 |
| ATOM | 2254 | CA | GLU | 1114 | 6.899 | 5.293 | 14.908 | 1.00 | 34.92 |
| ATOM | 2255 | CB | GLU | 1114 | 5.368 | 5.169 | 15.052 | 1.00 | 39.25 |
| ATOM | 2256 | CG | GLU | 1114 | 5.108 | 3.694 | 14.636 | 1.00 | 46.99 |
| ATOM | 2257 | CD | GLU | 1114 | 5.688 | 3.448 | 13.212 | 1.00 | 52.05 |
| ATOM | 2258 | OE1 | GLU | 1114 | 6.786 | 2.852 | 13.089 | 1.00 | 55.29 |
| ATOM | 2259 | OE2 | GLU | 1114 | 5.029 | 3.881 | 12.245 | 1.00 | 53.93 |
| ATOM | 2260 | C | GLU | 1114 | 7.354 | 6.728 | 14.900 | 1.00 | 31.33 |
| ATOM | 2261 | O | GLU | 1114 | 7.778 | 7.265 | 13.883 | 1.00 | 30.77 |
| ATOM | 2262 | N | PHE | 1115 | 7.355 | 7.357 | 16.068 | 1.00 | 30.17 |
| ATOM | 2263 | H | PHE | 1115 | 6.973 | 6.910 | 16.832 | 1.00 | 0.00 |
| ATOM | 2264 | CA | PHE | 1115 | 7.813 | 8.742 | 16.205 | 1.00 | 26.65 |
| ATOM | 2265 | CB | PHE | 1115 | 7.640 | 9.143 | 17.687 | 1.00 | 27.82 |
| ATOM | 2266 | CG | PHE | 1115 | 8.561 | 10.254 | 18.034 | 1.00 | 28.06 |
| ATOM | 2267 | CD1 | PHE | 1115 | 9.789 | 9.964 | 18.635 | 1.00 | 26.19 |
| ATOM | 2268 | CD2 | PHE | 1115 | 8.236 | 11.599 | 17.726 | 1.00 | 28.22 |
| ATOM | 2269 | CE1 | PHE | 1115 | 10.695 | 11.015 | 18.905 | 1.00 | 28.74 |
| ATOM | 2270 | CE2 | PHE | 1115 | 9.158 | 12.641 | 18.002 | 1.00 | 25.90 |
| ATOM | 2271 | CZ | PHE | 1115 | 10.391 | 12.348 | 18.588 | 1.00 | 25.10 |
| ATOM | 2272 | C | PHE | 1115 | 9.249 | 8.831 | 15.723 | 1.00 | 25.32 |
| ATOM | 2273 | O | PHE | 1115 | 9.550 | 9.638 | 14.845 | 1.00 | 23.89 |
| ATOM | 2274 | N | CYS | 1116 | 10.149 | 7.978 | 16.204 | 1.00 | 25.83 |
| ATOM | 2275 | H | CYS | 1116 | 9.879 | 7.344 | 16.900 | 1.00 | 0.00 |
| ATOM | 2276 | CA | CYS | 1116 | 11.556 | 8.041 | 15.751 | 1.00 | 28.80 |
| ATOM | 2277 | CB | CYS | 1116 | 12.394 | 7.010 | 16.520 | 1.00 | 28.75 |
| ATOM | 2278 | SG | CYS | 1116 | 12.645 | 7.395 | 18.267 | 1.00 | 30.36 |
| ATOM | 2279 | C | CYS | 1116 | 11.666 | 7.799 | 14.230 | 1.00 | 30.80 |
| ATOM | 2280 | O | CYS | 1116 | 12.449 | 8.417 | 13.474 | 1.00 | 28.85 |
| ATOM | 2281 | N | ARG | 1117 | 10.783 | 6.924 | 13.730 | 1.00 | 33.67 |
| ATOM | 2282 | H | ARG | 1117 | 10.146 | 6.493 | 14.330 | 1.00 | 0.00 |
| ATOM | 2283 | CA | ARG | 1117 | 10.771 | 6.621 | 12.295 | 1.00 | 35.49 |
| ATOM | 2284 | CB | ARG | 1117 | 9.774 | 5.503 | 12.045 | 1.00 | 39.95 |
| ATOM | 2285 | CG | ARG | 1117 | 9.836 | 4.992 | 10.599 | 1.00 | 46.19 |
| ATOM | 2286 | CD | ARG | 1117 | 8.574 | 4.140 | 10.206 | 1.00 | 47.85 |
| ATOM | 2287 | NE | ARG | 1117 | 7.558 | 4.875 | 9.393 | 1.00 | 48.96 |
| ATOM | 2288 | HE | ARG | 1117 | 7.559 | 4.756 | 8.421 | 1.00 | 0.00 |
| ATOM | 2289 | CZ | ARG | 1117 | 6.615 | 5.654 | 9.934 | 1.00 | 50.65 |
| ATOM | 2290 | NH1 | ARG | 1117 | 5.708 | 6.285 | 9.165 | 1.00 | 50.95 |
| ATOM | 2291 | HH11 | ARG | 1117 | 5.720 | 6.166 | 8.172 | 1.00 | 0.00 |
| ATOM | 2292 | HH12 | ARG | 1117 | 5.010 | 6.859 | 9.596 | 1.00 | 0.00 |
| ATOM | 2293 | NH2 | ARG | 1117 | 6.588 | 5.816 | 11.266 | 1.00 | 54.46 |
| ATOM | 2294 | HH21 | ARG | 1117 | 7.270 | 5.367 | 11.844 | 1.00 | 0.00 |
| ATOM | 2295 | HH22 | ARG | 1117 | 5.885 | 6.384 | 11.694 | 1.00 | 0.00 |
| ATOM | 2296 | C | ARG | 1117 | 10.377 | 7.860 | 11.512 | 1.00 | 34.51 |
| ATOM | 2297 | O | ARG | 1117 | 11.119 | 8.316 | 10.641 | 1.00 | 35.21 |
| ATOM | 2298 | N | ARG | 1118 | 9.236 | 8.470 | 11.845 | 1.00 | 33.52 |
| ATOM | 2299 | H | ARG | 1118 | 8.718 | 8.075 | 12.569 | 1.00 | 0.00 |
| ATOM | 2300 | CA | ARG | 1118 | 8.730 | 9.686 | 11.168 | 1.00 | 33.81 |
| ATOM | 2301 | CB | ARG | 1118 | 7.454 | 10.049 | 11.863 | 1.00 | 35.55 |
| ATOM | 2302 | CG | ARG | 1118 | 6.330 | 9.252 | 11.181 | 1.00 | 42.27 |
| ATOM | 2303 | CD | ARG | 1118 | 5.222 | 8.813 | 12.162 | 1.00 | 47.82 |
| ATOM | 2304 | NE | ARG | 1118 | 4.968 | 9.973 | 13.022 | 1.00 | 54.00 |
| ATOM | 2305 | HE | ARG | 1118 | 4.938 | 10.845 | 12.584 | 1.00 | 0.00 |
| ATOM | 2306 | CZ | ARG | 1118 | 4.733 | 9.947 | 14.345 | 1.00 | 53.99 |
| ATOM | 2307 | NH1 | ARG | 1118 | 4.561 | 11.142 | 14.906 | 1.00 | 56.57 |
| ATOM | 2308 | HH11 | ARG | 1118 | 4.615 | 11.962 | 14.339 | 1.00 | 0.00 |
| ATOM | 2309 | HH12 | ARG | 1118 | 4.383 | 11.201 | 15.884 | 1.00 | 0.00 |
| ATOM | 2310 | NH2 | ARG | 1118 | 4.609 | 8.840 | 15.083 | 1.00 | 53.66 |
| ATOM | 2311 | HH21 | ARG | 1118 | 4.681 | 7.932 | 14.666 | 1.00 | 0.00 |
| ATOM | 2312 | HH22 | ARG | 1118 | 4.443 | 8.923 | 16.062 | 1.00 | 0.00 |
| ATOM | 2313 | C | ARG | 1118 | 9.713 | 10.851 | 11.147 | 1.00 | 32.88 |
| ATOM | 2314 | O | ARG | 1118 | 9.930 | 11.566 | 10.166 | 1.00 | 30.09 |
| ATOM | 2315 | N | LEU | 1119 | 10.345 | 11.039 | 12.290 | 1.00 | 32.67 |
| ATOM | 2316 | H | LEU | 1119 | 10.127 | 10.447 | 13.039 | 1.00 | 0.00 |
| ATOM | 2317 | CA | LEU | 1119 | 11.364 | 12.090 | 12.425 | 1.00 | 32.13 |
| ATOM | 2318 | CB | LEU | 1119 | 11.988 | 12.013 | 13.776 | 1.00 | 31.96 |
| ATOM | 2319 | CG | LEU | 1119 | 12.412 | 13.362 | 14.257 | 1.00 | 33.97 |
| ATOM | 2320 | CD1 | LEU | 1119 | 11.121 | 14.143 | 14.699 | 1.00 | 34.08 |
| ATOM | 2321 | CD2 | LEU | 1119 | 13.520 | 13.155 | 15.338 | 1.00 | 35.42 |
| ATOM | 2322 | C | LEU | 1119 | 12.464 | 11.857 | 11.398 | 1.00 | 31.07 |
| ATOM | 2323 | O | LEU | 1119 | 12.924 | 12.769 | 10.718 | 1.00 | 29.19 |
| ATOM | 2324 | N | LYS | 1120 | 12.970 | 10.617 | 11.323 | 1.00 | 33.38 |
| ATOM | 2325 | H | LYS | 1120 | 12.638 | 9.907 | 11.921 | 1.00 | 0.00 |
| ATOM | 2326 | CA | LYS | 1120 | 14.028 | 10.339 | 10.319 | 1.00 | 35.07 |
| ATOM | 2327 | CB | LYS | 1120 | 14.531 | 8.874 | 10.434 | 1.00 | 36.62 |
| ATOM | 2328 | CG | LYS | 1120 | 15.946 | 8.709 | 9.846 | 1.00 | 39.11 |
| ATOM | 2329 | CD | LYS | 1120 | 16.657 | 7.343 | 10.127 | 1.00 | 43.55 |
| ATOM | 2330 | CE | LYS | 1120 | 18.212 | 7.379 | 9.780 | 1.00 | 47.43 |
| ATOM | 2331 | NZ | LYS | 1120 | 19.097 | 6.574 | 10.677 | 1.00 | 43.59 |
| ATOM | 2332 | HZ1 | LYS | 1120 | 18.669 | 5.633 | 10.801 | 1.00 | 0.00 |
| ATOM | 2333 | HZ2 | LYS | 1120 | 19.154 | 7.043 | 11.603 | 1.00 | 0.00 |
| ATOM | 2334 | HZ3 | LYS | 1120 | 20.045 | 6.477 | 10.265 | 1.00 | 0.00 |
| ATOM | 2335 | C | LYS | 1120 | 13.426 | 10.583 | 8.937 | 1.00 | 34.22 |
| ATOM | 2336 | O | LYS | 1120 | 14.123 | 11.042 | 8.040 | 1.00 | 33.35 |
| ATOM | 2337 | N | GLU | 1121 | 12.132 | 10.311 | 8.718 | 1.00 | 35.16 |
| ATOM | 2338 | H | GLU | 1121 | 11.586 | 9.945 | 9.442 | 1.00 | 0.00 |
| ATOM | 2339 | CA | GLU | 1121 | 11.603 | 10.578 | 7.356 | 1.00 | 36.89 |
| ATOM | 2340 | CB | GLU | 1121 | 10.280 | 9.806 | 7.042 | 1.00 | 38.47 |
| ATOM | 2341 | CG | GLU | 1121 | 9.744 | 8.757 | 8.011 | 1.00 | 46.53 |
| ATOM | 2342 | CD | GLU | 1121 | 8.303 | 8.282 | 7.670 | 1.00 | 51.63 |
| ATOM | 2343 | OE1 | GLU | 1121 | 7.387 | 9.126 | 7.551 | 1.00 | 51.95 |
| ATOM | 2344 | OE2 | GLU | 1121 | 8.120 | 7.052 | 7.541 | 1.00 | 53.65 |
| ATOM | 2345 | C | GLU | 1121 | 11.337 | 12.059 | 7.007 | 1.00 | 35.52 |
| ATOM | 2346 | O | GLU | 1121 | 10.960 | 12.380 | 5.865 | 1.00 | 37.05 |
| ATOM | 2347 | N | GLY | 1122 | 11.451 | 12.975 | 7.985 | 1.00 | 32.29 |
| ATOM | 2348 | H | GLY | 1122 | 11.743 | 12.674 | 8.873 | 1.00 | 0.00 |
| ATOM | 2349 | CA | GLY | 1122 | 11.215 | 14.420 | 7.717 | 1.00 | 28.66 |
| ATOM | 2350 | C | GLY | 1122 | 10.109 | 15.044 | 8.578 | 1.00 | 26.39 |
| ATOM | 2351 | O | GLY | 1122 | 9.845 | 16.226 | 8.516 | 1.00 | 27.28 |
| ATOM | 2352 | N | THR | 1123 | 9.360 | 14.317 | 9.392 | 1.00 | 26.01 |
| ATOM | 2353 | H | THR | 1123 | 9.452 | 13.351 | 9.359 | 1.00 | 0.00 |
| ATOM | 2354 | CA | THR | 1123 | 8.291 | 14.903 | 10.252 | 1.00 | 25.55 |
| ATOM | 2355 | CB | THR | 1123 | 7.518 | 13.840 | 11.029 | 1.00 | 24.70 |
| ATOM | 2356 | OG1 | THR | 1123 | 7.052 | 12.891 | 10.098 | 1.00 | 30.21 |
| ATOM | 2357 | HG1 | THR | 1123 | 6.582 | 12.213 | 10.583 | 1.00 | 0.00 |
| ATOM | 2358 | CG2 | THR | 1123 | 6.319 | 14.391 | 11.802 | 1.00 | 24.78 |
| ATOM | 2359 | C | THR | 1123 | 8.913 | 15.842 | 11.304 | 1.00 | 24.92 |
| ATOM | 2360 | O | THR | 1123 | 9.829 | 15.489 | 12.061 | 1.00 | 22.27 |
| ATOM | 2361 | N | ARG | 1124 | 8.308 | 17.024 | 11.392 | 1.00 | 23.65 |
| ATOM | 2362 | H | ARG | 1124 | 7.508 | 17.096 | 10.852 | 1.00 | 0.00 |
| ATOM | 2363 | CA | ARG | 1124 | 8.708 | 18.125 | 12.278 | 1.00 | 21.21 |
| ATOM | 2364 | CB | ARG | 1124 | 9.364 | 19.225 | 11.460 | 1.00 | 19.08 |
| ATOM | 2365 | CG | ARG | 1124 | 10.623 | 18.885 | 10.671 | 1.00 | 17.71 |
| ATOM | 2366 | CD | ARG | 1124 | 11.682 | 18.142 | 11.493 | 1.00 | 17.97 |
| ATOM | 2367 | NE | ARG | 1124 | 12.912 | 17.835 | 10.736 | 1.00 | 18.87 |
| ATOM | 2368 | HE | ARG | 1124 | 13.484 | 18.583 | 10.470 | 1.00 | 0.00 |
| ATOM | 2369 | CZ | ARG | 1124 | 13.269 | 16.596 | 10.400 | 1.00 | 15.76 |
| ATOM | 2370 | NH1 | ARG | 1124 | 12.529 | 15.550 | 10.693 | 1.00 | 16.69 |
| ATOM | 2371 | HH11 | ARG | 1124 | 11.679 | 15.667 | 11.201 | 1.00 | 0.00 |
| ATOM | 2372 | HH12 | ARG | 1124 | 12.831 | 14.640 | 10.419 | 1.00 | 0.00 |
| ATOM | 2373 | NH2 | ARG | 1124 | 14.419 | 16.456 | 9.773 | 1.00 | 14.99 |
| ATOM | 2374 | HH21 | ARG | 1124 | 14.975 | 17.260 | 9.567 | 1.00 | 0.00 |
| ATOM | 2375 | HH22 | ARG | 1124 | 14.733 | 15.566 | 9.468 | 1.00 | 0.00 |
| ATOM | 2376 | C | ARG | 1124 | 7.519 | 18.752 | 12.987 | 1.00 | 22.80 |
| ATOM | 2377 | O | ARG | 1124 | 6.386 | 18.662 | 12.471 | 1.00 | 24.71 |
| ATOM | 2378 | N | MET | 1125 | 7.695 | 19.442 | 14.133 | 1.00 | 19.50 |
| ATOM | 2379 | H | MET | 1125 | 8.563 | 19.532 | 14.548 | 1.00 | 0.00 |
| ATOM | 2380 | CA | MET | 1125 | 6.565 | 20.124 | 14.787 | 1.00 | 17.04 |
| ATOM | 2381 | CB | MET | 1125 | 6.951 | 20.707 | 16.147 | 1.00 | 15.53 |
| ATOM | 2382 | CG | MET | 1125 | 7.161 | 19.629 | 17.197 | 1.00 | 16.08 |
| ATOM | 2383 | SD | MET | 1125 | 7.525 | 20.324 | 18.808 | 1.00 | 16.93 |
| ATOM | 2384 | CE | MET | 1125 | 9.326 | 20.396 | 18.598 | 1.00 | 10.08 |
| ATOM | 2385 | C | MET | 1125 | 6.074 | 21.293 | 13.957 | 1.00 | 16.72 |
| ATOM | 2386 | O | MET | 1125 | 6.774 | 21.845 | 13.102 | 1.00 | 15.05 |
| ATOM | 2387 | N | ARG | 1126 | 4.814 | 21.602 | 14.214 | 1.00 | 20.23 |
| ATOM | 2388 | H | ARG | 1126 | 4.280 | 20.958 | 14.711 | 1.00 | 0.00 |
| ATOM | 2389 | CA | ARG | 1126 | 4.098 | 22.766 | 13.615 | 1.00 | 24.80 |
| ATOM | 2390 | CB | ARG | 1126 | 2.513 | 22.717 | 13.801 | 1.00 | 27.74 |
| ATOM | 2391 | CG | ARG | 1126 | 1.796 | 22.643 | 15.270 | 1.00 | 40.41 |
| ATOM | 2392 | CD | ARG | 1126 | 1.330 | 23.799 | 16.352 | 1.00 | 44.42 |
| ATOM | 2393 | NE | ARG | 1126 | 1.840 | 23.720 | 17.788 | 1.00 | 44.59 |
| ATOM | 2394 | HE | ARG | 1126 | 2.710 | 23.301 | 17.949 | 1.00 | 0.00 |
| ATOM | 2395 | CZ | ARG | 1126 | 1.173 | 24.261 | 18.888 | 1.00 | 46.74 |
| ATOM | 2396 | NH1 | ARG | 1126 | 1.706 | 24.153 | 20.125 | 1.00 | 41.03 |
| ATOM | 2397 | HH11 | ARG | 1126 | 2.587 | 23.698 | 20.247 | 1.00 | 0.00 |
| ATOM | 2398 | HH12 | ARG | 1126 | 1.224 | 24.531 | 20.917 | 1.00 | 0.00 |
| ATOM | 2399 | NH2 | ARG | 1126 | −0.043 | 24.888 | 18.825 | 1.00 | 43.10 |
| ATOM | 2400 | HH21 | ARG | 1126 | −0.511 | 24.980 | 17.946 | 1.00 | 0.00 |
| ATOM | 2401 | HH22 | ARG | 1126 | −0.460 | 25.252 | 19.658 | 1.00 | 0.00 |
| ATOM | 2402 | C | ARG | 1126 | 4.573 | 24.028 | 14.353 | 1.00 | 22.66 |
| ATOM | 2403 | O | ARG | 1126 | 5.033 | 23.957 | 15.502 | 1.00 | 22.72 |
| ATOM | 2404 | N | ALA | 1127 | 4.330 | 25.184 | 13.782 | 1.00 | 19.86 |
| ATOM | 2405 | H | ALA | 1127 | 3.969 | 25.160 | 12.873 | 1.00 | 0.00 |
| ATOM | 2406 | CA | ALA | 1127 | 4.692 | 26.452 | 14.414 | 1.00 | 18.73 |
| ATOM | 2407 | CB | ALA | 1127 | 4.156 | 27.642 | 13.599 | 1.00 | 18.79 |
| ATOM | 2408 | C | ALA | 1127 | 4.085 | 26.546 | 15.788 | 1.00 | 17.90 |
| ATOM | 2409 | O | ALA | 1127 | 2.890 | 26.307 | 15.940 | 1.00 | 18.45 |
| ATOM | 2410 | N | PRO | 1128 | 4.829 | 26.952 | 16.813 | 1.00 | 17.07 |
| ATOM | 2411 | CD | PRO | 1128 | 6.275 | 27.183 | 16.900 | 1.00 | 14.67 |
| ATOM | 2412 | CA | PRO | 1128 | 4.176 | 27.199 | 18.109 | 1.00 | 16.71 |
| ATOM | 2413 | CB | PRO | 1128 | 5.396 | 27.204 | 19.076 | 1.00 | 15.26 |
| ATOM | 2414 | CG | PRO | 1128 | 6.446 | 27.901 | 18.272 | 1.00 | 11.48 |
| ATOM | 2415 | C | PRO | 1128 | 3.287 | 28.487 | 18.063 | 1.00 | 14.59 |
| ATOM | 2416 | O | PRO | 1128 | 3.460 | 29.386 | 17.248 | 1.00 | 12.19 |
| ATOM | 2417 | N | ASP | 1129 | 2.383 | 28.642 | 19.001 | 1.00 | 15.17 |
| ATOM | 2418 | H | ASP | 1129 | 2.389 | 27.997 | 19.732 | 1.00 | 0.00 |
| ATOM | 2419 | CA | ASP | 1129 | 1.436 | 29.785 | 19.066 | 1.00 | 16.31 |
| ATOM | 2420 | CB | ASP | 1129 | 0.523 | 29.688 | 20.294 | 1.00 | 14.33 |
| ATOM | 2421 | CG | ASP | 1129 | −0.453 | 28.512 | 20.137 | 1.00 | 15.22 |
| ATOM | 2422 | OD1 | ASP | 1129 | −1.038 | 28.111 | 21.128 | 1.00 | 16.41 |
| ATOM | 2423 | OD2 | ASP | 1129 | −0.629 | 27.952 | 19.055 | 1.00 | 20.01 |
| ATOM | 2424 | C | ASP | 1129 | 1.935 | 31.183 | 19.032 | 1.00 | 16.10 |
| ATOM | 2425 | O | ASP | 1129 | 1.279 | 32.051 | 18.486 | 1.00 | 14.67 |
| ATOM | 2426 | N | TYR | 1130 | 3.096 | 31.402 | 19.642 | 1.00 | 14.64 |
| ATOM | 2427 | H | TYR | 1130 | 3.599 | 30.639 | 19.992 | 1.00 | 0.00 |
| ATOM | 2428 | CA | TYR | 1130 | 3.697 | 32.749 | 19.790 | 1.00 | 14.41 |
| ATOM | 2429 | CB | TYR | 1130 | 4.082 | 32.905 | 21.266 | 1.00 | 12.13 |
| ATOM | 2430 | CG | TYR | 1130 | 3.012 | 32.420 | 22.146 | 1.00 | 11.66 |
| ATOM | 2431 | CD1 | TYR | 1130 | 3.055 | 31.130 | 22.724 | 1.00 | 13.43 |
| ATOM | 2432 | CE1 | TYR | 1130 | 2.039 | 30.713 | 23.605 | 1.00 | 13.95 |
| ATOM | 2433 | CD2 | TYR | 1130 | 1.969 | 33.284 | 22.435 | 1.00 | 12.02 |
| ATOM | 2434 | CE2 | TYR | 1130 | 0.965 | 32.865 | 23.306 | 1.00 | 13.87 |
| ATOM | 2435 | CZ | TYR | 1130 | 0.994 | 31.602 | 23.890 | 1.00 | 12.14 |
| ATOM | 2436 | OH | TYR | 1130 | −0.061 | 31.232 | 24.710 | 1.00 | 14.72 |
| ATOM | 2437 | HH | TYR | 1130 | −0.719 | 31.929 | 24.769 | 1.00 | 0.00 |
| ATOM | 2438 | C | TYR | 1130 | 4.896 | 33.066 | 18.902 | 1.00 | 15.45 |
| ATOM | 2439 | O | TYR | 1130 | 5.588 | 34.085 | 19.037 | 1.00 | 16.56 |
| ATOM | 2440 | N | THR | 1131 | 5.179 | 32.139 | 17.997 | 1.00 | 15.67 |
| ATOM | 2441 | H | THR | 1131 | 4.550 | 31.400 | 17.878 | 1.00 | 0.00 |
| ATOM | 2442 | CA | THR | 1131 | 6.310 | 32.322 | 17.081 | 1.00 | 16.24 |
| ATOM | 2443 | CB | THR | 1131 | 6.586 | 31.032 | 16.257 | 1.00 | 18.38 |
| ATOM | 2444 | OG1 | THR | 1131 | 7.873 | 31.186 | 15.656 | 1.00 | 19.66 |
| ATOM | 2445 | HG1 | THR | 1131 | 8.031 | 30.429 | 15.084 | 1.00 | 0.00 |
| ATOM | 2446 | CG2 | THR | 1131 | 5.555 | 30.777 | 15.087 | 1.00 | 17.32 |
| ATOM | 2447 | C | THR | 1131 | 6.118 | 33.435 | 16.049 | 1.00 | 19.05 |
| ATOM | 2448 | O | THR | 1131 | 5.037 | 33.987 | 15.832 | 1.00 | 17.09 |
| ATOM | 2449 | N | THR | 1132 | 7.243 | 33.856 | 15.486 | 1.00 | 19.47 |
| ATOM | 2450 | H | THR | 1132 | 8.082 | 33.449 | 15.785 | 1.00 | 0.00 |
| ATOM | 2451 | CA | THR | 1132 | 7.218 | 34.782 | 14.355 | 1.00 | 20.07 |
| ATOM | 2452 | CB | THR | 1132 | 8.299 | 35.793 | 14.356 | 1.00 | 18.50 |
| ATOM | 2453 | OG1 | THR | 1132 | 9.539 | 35.079 | 14.342 | 1.00 | 20.30 |
| ATOM | 2454 | HG1 | THR | 1132 | 10.260 | 35.692 | 14.572 | 1.00 | 0.00 |
| ATOM | 2455 | CG2 | THR | 1132 | 8.125 | 36.760 | 15.523 | 1.00 | 18.43 |
| ATOM | 2456 | C | THR | 1132 | 7.549 | 33.846 | 13.177 | 1.00 | 20.63 |
| ATOM | 2457 | O | THR | 1132 | 8.180 | 32.789 | 13.320 | 1.00 | 21.49 |
| ATOM | 2458 | N | PRO | 1133 | 7.215 | 34.186 | 11.948 | 1.00 | 22.80 |
| ATOM | 2459 | CD | PRO | 1133 | 6.541 | 35.444 | 11.481 | 1.00 | 24.91 |
| ATOM | 2460 | CA | PRO | 1133 | 7.522 | 33.272 | 10.803 | 1.00 | 21.51 |
| ATOM | 2461 | CB | PRO | 1133 | 7.049 | 34.040 | 9.548 | 1.00 | 23.91 |
| ATOM | 2462 | CG | PRO | 1133 | 6.002 | 35.022 | 10.105 | 1.00 | 25.43 |
| ATOM | 2463 | C | PRO | 1133 | 8.961 | 32.922 | 10.709 | 1.00 | 16.88 |
| ATOM | 2464 | O | PRO | 1133 | 9.371 | 31.816 | 10.397 | 1.00 | 14.90 |
| ATOM | 2465 | N | GLU | 1134 | 9.796 | 33.927 | 10.925 | 1.00 | 19.41 |
| ATOM | 2466 | H | GLU | 1134 | 9.442 | 34.817 | 11.118 | 1.00 | 0.00 |
| ATOM | 2467 | CA | GLU | 1134 | 11.251 | 33.653 | 10.792 | 1.00 | 22.25 |
| ATOM | 2468 | CB | GLU | 1134 | 12.042 | 34.978 | 10.629 | 1.00 | 25.85 |
| ATOM | 2469 | CG | GLU | 1134 | 11.568 | 36.278 | 11.293 | 1.00 | 36.01 |
| ATOM | 2470 | CD | GLU | 1134 | 10.318 | 36.945 | 10.696 | 1.00 | 38.07 |
| ATOM | 2471 | OE1 | GLU | 1134 | 9.423 | 37.361 | 11.432 | 1.00 | 40.95 |
| ATOM | 2472 | OE2 | GLU | 1134 | 10.251 | 37.087 | 9.487 | 1.00 | 42.46 |
| ATOM | 2473 | C | GLU | 1134 | 11.784 | 32.825 | 11.931 | 1.00 | 20.02 |
| ATOM | 2474 | O | GLU | 1134 | 12.721 | 32.040 | 11.784 | 1.00 | 19.02 |
| ATOM | 2475 | N | MET | 1135 | 11.158 | 32.970 | 13.101 | 1.00 | 19.15 |
| ATOM | 2476 | H | MET | 1135 | 10.395 | 33.575 | 13.223 | 1.00 | 0.00 |
| ATOM | 2477 | CA | MET | 1135 | 11.603 | 32.100 | 14.195 | 1.00 | 20.10 |
| ATOM | 2478 | CB | MET | 1135 | 10.963 | 32.486 | 15.554 | 1.00 | 20.02 |
| ATOM | 2479 | CG | MET | 1135 | 11.669 | 33.690 | 16.279 | 1.00 | 24.47 |
| ATOM | 2480 | SD | MET | 1135 | 13.468 | 33.509 | 16.579 | 1.00 | 25.23 |
| ATOM | 2481 | CE | MET | 1135 | 13.458 | 31.840 | 16.968 | 1.00 | 12.17 |
| ATOM | 2482 | C | MET | 1135 | 11.213 | 30.649 | 13.829 | 1.00 | 18.06 |
| ATOM | 2483 | O | MET | 1135 | 12.023 | 29.738 | 14.036 | 1.00 | 16.98 |
| ATOM | 2484 | N | TYR | 1136 | 9.985 | 30.388 | 13.298 | 1.00 | 16.13 |
| ATOM | 2485 | H | TYR | 1136 | 9.352 | 31.139 | 13.207 | 1.00 | 0.00 |
| ATOM | 2486 | CA | TYR | 1136 | 9.638 | 29.019 | 12.897 | 1.00 | 15.52 |
| ATOM | 2487 | CB | TYR | 1136 | 8.165 | 29.002 | 12.444 | 1.00 | 16.13 |
| ATOM | 2488 | CG | TYR | 1136 | 7.848 | 27.570 | 12.212 | 1.00 | 17.93 |
| ATOM | 2489 | CD1 | TYR | 1136 | 8.015 | 26.574 | 13.193 | 1.00 | 18.11 |
| ATOM | 2490 | CE1 | TYR | 1136 | 7.662 | 25.229 | 12.931 | 1.00 | 16.48 |
| ATOM | 2491 | CD2 | TYR | 1136 | 7.326 | 27.198 | 10.955 | 1.00 | 19.38 |
| ATOM | 2492 | CE2 | TYR | 1136 | 6.971 | 25.860 | 10.705 | 1.00 | 17.31 |
| ATOM | 2493 | CZ | TYR | 1136 | 7.139 | 24.877 | 11.678 | 1.00 | 18.42 |
| ATOM | 2494 | OH | TYR | 1136 | 6.751 | 23.597 | 11.363 | 1.00 | 18.68 |
| ATOM | 2495 | HH | TYR | 1136 | 6.419 | 23.638 | 10.458 | 1.00 | 0.00 |
| ATOM | 2496 | C | TYR | 1136 | 10.591 | 28.453 | 11.799 | 1.00 | 16.25 |
| ATOM | 2497 | O | TYR | 1136 | 11.008 | 27.298 | 11.772 | 1.00 | 15.52 |
| ATOM | 2498 | N | GLN | 1137 | 10.995 | 29.244 | 10.840 | 1.00 | 16.78 |
| ATOM | 2499 | H | GLN | 1137 | 10.617 | 30.149 | 10.786 | 1.00 | 0.00 |
| ATOM | 2500 | CA | GLN | 1137 | 11.929 | 28.734 | 9.830 | 1.00 | 18.33 |
| ATOM | 2501 | CB | GLN | 1137 | 12.050 | 29.768 | 8.669 | 1.00 | 20.85 |
| ATOM | 2502 | CG | GLN | 1137 | 13.014 | 29.300 | 7.521 | 1.00 | 22.56 |
| ATOM | 2503 | CD | GLN | 1137 | 12.508 | 27.975 | 6.970 | 1.00 | 23.45 |
| ATOM | 2504 | OE1 | GLN | 1137 | 11.323 | 27.895 | 6.611 | 1.00 | 22.38 |
| ATOM | 2505 | NE2 | GLN | 1137 | 13.318 | 26.908 | 6.954 | 1.00 | 18.71 |
| ATOM | 2506 | HE21 | GLN | 1137 | 14.218 | 26.994 | 7.296 | 1.00 | 0.00 |
| ATOM | 2507 | HE22 | GLN | 1137 | 12.983 | 26.056 | 6.592 | 1.00 | 0.00 |
| ATOM | 2508 | C | GLN | 1137 | 13.287 | 28.453 | 10.469 | 1.00 | 15.66 |
| ATOM | 2509 | O | GLN | 1137 | 14.026 | 27.602 | 10.011 | 1.00 | 14.23 |
| ATOM | 2510 | N | THR | 1138 | 13.675 | 29.222 | 11.472 | 1.00 | 14.73 |
| ATOM | 2511 | H | THR | 1138 | 13.136 | 30.003 | 11.705 | 1.00 | 0.00 |
| ATOM | 2512 | CA | THR | 1138 | 14.944 | 28.975 | 12.151 | 1.00 | 15.71 |
| ATOM | 2513 | CB | THR | 1138 | 15.224 | 30.173 | 13.131 | 1.00 | 15.63 |
| ATOM | 2514 | OG1 | THR | 1138 | 15.435 | 31.348 | 12.319 | 1.00 | 17.48 |
| ATOM | 2515 | HG1 | THR | 1138 | 15.547 | 32.100 | 12.913 | 1.00 | 0.00 |
| ATOM | 2516 | CG2 | THR | 1138 | 16.428 | 29.906 | 14.072 | 1.00 | 13.26 |
| ATOM | 2517 | C | THR | 1138 | 14.819 | 27.625 | 12.867 | 1.00 | 15.46 |
| ATOM | 2518 | O | THR | 1138 | 15.753 | 26.808 | 12.815 | 1.00 | 18.78 |
| ATOM | 2519 | N | MET | 1139 | 13.684 | 27.320 | 13.492 | 1.00 | 14.16 |
| ATOM | 2520 | H | MET | 1139 | 12.993 | 28.015 | 13.563 | 1.00 | 0.00 |
| ATOM | 2521 | CA | MET | 1139 | 13.466 | 26.026 | 14.119 | 1.00 | 12.32 |
| ATOM | 2522 | CB | MET | 1139 | 12.083 | 25.928 | 14.674 | 1.00 | 10.25 |
| ATOM | 2523 | CG | MET | 1139 | 11.885 | 26.841 | 15.835 | 1.00 | 10.46 |
| ATOM | 2524 | SD | MET | 1139 | 10.152 | 26.772 | 16.313 | 1.00 | 13.10 |
| ATOM | 2525 | CE | MET | 1139 | 9.943 | 28.438 | 16.985 | 1.00 | 8.95 |
| ATOM | 2526 | C | MET | 1139 | 13.616 | 24.956 | 13.031 | 1.00 | 14.59 |
| ATOM | 2527 | O | MET | 1139 | 14.348 | 23.984 | 13.151 | 1.00 | 15.84 |
| ATOM | 2528 | N | LEU | 1140 | 12.976 | 25.099 | 11.864 | 1.00 | 16.16 |
| ATOM | 2529 | H | LEU | 1140 | 12.378 | 25.867 | 11.760 | 1.00 | 0.00 |
| ATOM | 2530 | CA | LEU | 1140 | 13.102 | 24.081 | 10.763 | 1.00 | 16.47 |
| ATOM | 2531 | CB | LEU | 1140 | 12.145 | 24.485 | 9.595 | 1.00 | 15.66 |
| ATOM | 2532 | CG | LEU | 1140 | 10.628 | 24.493 | 9.904 | 1.00 | 15.64 |
| ATOM | 2533 | CD1 | LEU | 1140 | 9.912 | 24.934 | 8.609 | 1.00 | 18.67 |
| ATOM | 2534 | CD2 | LEU | 1140 | 10.084 | 23.111 | 10.285 | 1.00 | 14.82 |
| ATOM | 2535 | C | LEU | 1140 | 14.565 | 23.940 | 10.267 | 1.00 | 15.15 |
| ATOM | 2536 | O | LEU | 1140 | 15.060 | 22.860 | 9.965 | 1.00 | 15.22 |
| ATOM | 2537 | N | ASP | 1141 | 15.331 | 25.031 | 10.184 | 1.00 | 15.81 |
| ATOM | 2538 | H | ASP | 1141 | 14.910 | 25.883 | 10.415 | 1.00 | 0.00 |
| ATOM | 2539 | CA | ASP | 1141 | 16.725 | 24.983 | 9.775 | 1.00 | 16.12 |
| ATOM | 2540 | CB | ASP | 1141 | 17.321 | 26.397 | 9.851 | 1.00 | 17.34 |
| ATOM | 2541 | CG | ASP | 1141 | 16.749 | 27.354 | 8.768 | 1.00 | 20.41 |
| ATOM | 2542 | OD1 | ASP | 1141 | 16.948 | 28.579 | 8.933 | 1.00 | 20.82 |
| ATOM | 2543 | OD2 | ASP | 1141 | 16.127 | 26.945 | 7.808 | 1.00 | 18.82 |
| ATOM | 2544 | C | ASP | 1141 | 17.504 | 24.075 | 10.719 | 1.00 | 18.57 |
| ATOM | 2545 | O | ASP | 1141 | 18.304 | 23.218 | 10.313 | 1.00 | 17.72 |
| ATOM | 2546 | N | CYS | 1142 | 17.301 | 24.343 | 12.035 | 1.00 | 17.65 |
| ATOM | 2547 | H | CYS | 1142 | 16.710 | 25.097 | 12.274 | 1.00 | 0.00 |
| ATOM | 2548 | CA | CYS | 1142 | 17.942 | 23.591 | 13.141 | 1.00 | 16.33 |
| ATOM | 2549 | CB | CYS | 1142 | 17.513 | 24.167 | 14.528 | 1.00 | 16.51 |
| ATOM | 2550 | SG | CYS | 1142 | 18.105 | 25.841 | 14.909 | 1.00 | 16.45 |
| ATOM | 2551 | C | CYS | 1142 | 17.554 | 22.150 | 13.075 | 1.00 | 16.22 |
| ATOM | 2552 | O | CYS | 1142 | 18.244 | 21.244 | 13.527 | 1.00 | 15.60 |
| ATOM | 2553 | N | TRP | 1143 | 16.377 | 21.885 | 12.548 | 1.00 | 16.70 |
| ATOM | 2554 | H | TRP | 1143 | 15.773 | 22.612 | 12.309 | 1.00 | 0.00 |
| ATOM | 2555 | CA | TRP | 1143 | 15.970 | 20.489 | 12.424 | 1.00 | 16.43 |
| ATOM | 2556 | CB | TRP | 1143 | 14.448 | 20.343 | 12.711 | 1.00 | 17.60 |
| ATOM | 2557 | CG | TRP | 1143 | 14.079 | 20.811 | 14.078 | 1.00 | 18.53 |
| ATOM | 2558 | CD2 | TRP | 1143 | 12.832 | 21.378 | 14.459 | 1.00 | 17.80 |
| ATOM | 2559 | CE2 | TRP | 1143 | 12.898 | 21.625 | 15.872 | 1.00 | 17.11 |
| ATOM | 2560 | CE3 | TRP | 1143 | 11.667 | 21.697 | 13.738 | 1.00 | 16.79 |
| ATOM | 2561 | CD1 | TRP | 1143 | 14.838 | 20.746 | 15.267 | 1.00 | 15.72 |
| ATOM | 2562 | NE1 | TRP | 1143 | 14.130 | 21.224 | 16.326 | 1.00 | 17.15 |
| ATOM | 2563 | HE1 | TRP | 1143 | 14.455 | 21.220 | 17.252 | 1.00 | 0.00 |
| ATOM | 2564 | CZ2 | TRP | 1143 | 11.790 | 22.194 | 16.517 | 1.00 | 14.44 |
| ATOM | 2565 | CZ3 | TRP | 1143 | 10.573 | 22.264 | 14.413 | 1.00 | 15.84 |
| ATOM | 2566 | CH2 | TRP | 1143 | 10.631 | 22.513 | 15.797 | 1.00 | 14.32 |
| ATOM | 2567 | C | TRP | 1143 | 16.299 | 19.868 | 11.038 | 1.00 | 17.99 |
| ATOM | 2568 | O | TRP | 1143 | 15.707 | 18.903 | 10.563 | 1.00 | 18.02 |
| ATOM | 2569 | N | HIS | 1144 | 17.235 | 20.433 | 10.335 | 1.00 | 16.73 |
| ATOM | 2570 | H | HIS | 1144 | 17.700 | 21.205 | 10.709 | 1.00 | 0.00 |
| ATOM | 2571 | CA | HIS | 1144 | 17.646 | 19.876 | 9.072 | 1.00 | 19.64 |
| ATOM | 2572 | C | HIS | 1144 | 18.089 | 18.401 | 9.270 | 1.00 | 21.67 |
| ATOM | 2573 | O | HIS | 1144 | 18.879 | 18.085 | 10.173 | 1.00 | 21.94 |
| ATOM | 2574 | CB | HIS | 1144 | 18.793 | 20.716 | 8.592 | 1.00 | 20.08 |
| ATOM | 2575 | CG | HIS | 1144 | 19.041 | 20.320 | 7.232 | 1.00 | 23.50 |
| ATOM | 2576 | ND1 | HIS | 1144 | 18.642 | 20.999 | 6.138 | 1.00 | 26.90 |
| ATOM | 2577 | HD1 | HIS | 1144 | 18.356 | 21.928 | 6.119 | 1.00 | 0.00 |
| ATOM | 2578 | CD2 | HIS | 1144 | 19.592 | 19.141 | 6.817 | 1.00 | 24.64 |
| ATOM | 2579 | NE2 | HIS | 1144 | 19.513 | 19.097 | 5.498 | 1.00 | 27.90 |
| ATOM | 2580 | CE1 | HIS | 1144 | 18.930 | 20.249 | 5.041 | 1.00 | 26.03 |
| ATOM | 2581 | N | GLY | 1145 | 17.632 | 17.435 | 8.439 | 1.00 | 22.33 |
| ATOM | 2582 | H | GLY | 1145 | 16.976 | 17.694 | 7.777 | 1.00 | 0.00 |
| ATOM | 2583 | CA | GLY | 1145 | 18.047 | 16.013 | 8.541 | 1.00 | 22.05 |
| ATOM | 2584 | C | GLY | 1145 | 19.601 | 15.890 | 8.596 | 1.00 | 22.81 |
| ATOM | 2585 | O | GLY | 1145 | 20.186 | 15.117 | 9.341 | 1.00 | 24.78 |
| ATOM | 2586 | N | GLU | 1146 | 20.303 | 16.602 | 7.743 | 1.00 | 24.29 |
| ATOM | 2587 | H | GLU | 1146 | 19.803 | 16.942 | 6.983 | 1.00 | 0.00 |
| ATOM | 2588 | CA | GLU | 1146 | 21.755 | 16.603 | 7.691 | 1.00 | 26.20 |
| ATOM | 2589 | CB | GLU | 1146 | 22.118 | 16.962 | 6.239 | 1.00 | 27.45 |
| ATOM | 2590 | CG | GLU | 1146 | 23.589 | 16.705 | 5.923 | 1.00 | 36.69 |
| ATOM | 2591 | CD | GLU | 1146 | 23.870 | 15.200 | 5.892 | 1.00 | 41.69 |
| ATOM | 2592 | OE1 | GLU | 1146 | 23.161 | 14.509 | 5.144 | 1.00 | 44.54 |
| ATOM | 2593 | OE2 | GLU | 1146 | 24.779 | 14.735 | 6.605 | 1.00 | 42.96 |
| ATOM | 2594 | C | GLU | 1146 | 22.449 | 17.527 | 8.721 | 1.00 | 23.48 |
| ATOM | 2595 | O | GLU | 1146 | 22.446 | 18.739 | 8.594 | 1.00 | 22.33 |
| ATOM | 2596 | N | PRO | 1147 | 23.204 | 16.987 | 9.688 | 1.00 | 22.51 |
| ATOM | 2597 | CD | PRO | 1147 | 23.494 | 15.556 | 9.888 | 1.00 | 21.48 |
| ATOM | 2598 | CA | PRO | 1147 | 23.864 | 17.778 | 10.738 | 1.00 | 21.57 |
| ATOM | 2599 | CB | PRO | 1147 | 24.769 | 16.795 | 11.495 | 1.00 | 18.99 |
| ATOM | 2600 | CG | PRO | 1147 | 24.110 | 15.461 | 11.276 | 1.00 | 20.48 |
| ATOM | 2601 | C | PRO | 1147 | 24.648 | 18.935 | 10.184 | 1.00 | 22.89 |
| ATOM | 2602 | O | PRO | 1147 | 24.603 | 20.073 | 10.696 | 1.00 | 21.52 |
| ATOM | 2603 | N | SER | 1148 | 25.434 | 18.626 | 9.113 | 1.00 | 24.00 |
| ATOM | 2604 | H | SER | 1148 | 25.449 | 17.700 | 8.795 | 1.00 | 0.00 |
| ATOM | 2605 | CA | SER | 1148 | 26.283 | 19.659 | 8.474 | 1.00 | 24.04 |
| ATOM | 2606 | CB | SER | 1148 | 27.250 | 19.075 | 7.406 | 1.00 | 24.88 |
| ATOM | 2607 | OG | SER | 1148 | 26.505 | 18.533 | 6.320 | 1.00 | 30.18 |
| ATOM | 2608 | HG | SER | 1148 | 27.111 | 18.140 | 5.672 | 1.00 | 0.00 |
| ATOM | 2609 | C | SER | 1148 | 25.460 | 20.732 | 7.830 | 1.00 | 22.01 |
| ATOM | 2610 | O | SER | 1148 | 25.941 | 21.845 | 7.695 | 1.00 | 23.03 |
| ATOM | 2611 | N | GLN | 1149 | 24.226 | 20.460 | 7.505 | 1.00 | 21.14 |
| ATOM | 2612 | H | GLN | 1149 | 23.917 | 19.546 | 7.638 | 1.00 | 0.00 |
| ATOM | 2613 | CA | GLN | 1149 | 23.337 | 21.467 | 6.891 | 1.00 | 23.98 |
| ATOM | 2614 | CB | GLN | 1149 | 22.449 | 20.673 | 5.947 | 1.00 | 27.89 |
| ATOM | 2615 | CG | GLN | 1149 | 23.244 | 20.163 | 4.691 | 1.00 | 36.15 |
| ATOM | 2616 | CD | GLN | 1149 | 23.193 | 21.306 | 3.715 | 1.00 | 40.22 |
| ATOM | 2617 | OE1 | GLN | 1149 | 22.430 | 21.298 | 2.760 | 1.00 | 40.87 |
| ATOM | 2618 | NE2 | GLN | 1149 | 23.878 | 22.403 | 3.947 | 1.00 | 43.28 |
| ATOM | 2619 | HE21 | GLN | 1149 | 24.438 | 22.533 | 4.728 | 1.00 | 0.00 |
| ATOM | 2620 | HE22 | GLN | 1149 | 23.800 | 23.112 | 3.279 | 1.00 | 0.00 |
| ATOM | 2621 | C | GLN | 1149 | 22.538 | 22.380 | 7.859 | 1.00 | 23.96 |
| ATOM | 2622 | O | GLN | 1149 | 21.833 | 23.336 | 7.474 | 1.00 | 23.55 |
| ATOM | 2623 | N | ARG | 1150 | 22.538 | 21.997 | 9.151 | 1.00 | 20.75 |
| ATOM | 2624 | H | ARG | 1150 | 22.983 | 21.157 | 9.404 | 1.00 | 0.00 |
| ATOM | 2625 | CA | ARG | 1150 | 21.906 | 22.836 | 10.198 | 1.00 | 18.29 |
| ATOM | 2626 | CB | ARG | 1150 | 21.818 | 22.098 | 11.546 | 1.00 | 15.51 |
| ATOM | 2627 | CG | ARG | 1150 | 20.988 | 20.826 | 11.426 | 1.00 | 15.22 |
| ATOM | 2628 | CD | ARG | 1150 | 21.030 | 19.939 | 12.665 | 1.00 | 16.45 |
| ATOM | 2629 | NE | ARG | 1150 | 20.388 | 18.685 | 12.305 | 1.00 | 15.94 |
| ATOM | 2630 | HE | ARG | 1150 | 19.665 | 18.733 | 11.658 | 1.00 | 0.00 |
| ATOM | 2631 | CZ | ARG | 1150 | 20.743 | 17.542 | 12.893 | 1.00 | 18.83 |
| ATOM | 2632 | NH1 | ARG | 1150 | 21.682 | 17.501 | 13.850 | 1.00 | 15.15 |
| ATOM | 2633 | HH11 | ARG | 1150 | 22.153 | 18.332 | 14.134 | 1.00 | 0.00 |
| ATOM | 2634 | HH12 | ARG | 1150 | 21.911 | 16.637 | 14.273 | 1.00 | 0.00 |
| ATOM | 2635 | NH2 | ARG | 1150 | 20.250 | 16.415 | 12.411 | 1.00 | 16.84 |
| ATOM | 2636 | HH21 | ARG | 1150 | 19.627 | 16.434 | 11.629 | 1.00 | 0.00 |
| ATOM | 2637 | HH22 | ARG | 1150 | 20.494 | 15.553 | 12.843 | 1.00 | 0.00 |
| ATOM | 2638 | C | ARG | 1150 | 22.815 | 24.069 | 10.395 | 1.00 | 17.42 |
| ATOM | 2639 | O | ARG | 1150 | 24.038 | 24.039 | 10.145 | 1.00 | 17.54 |
| ATOM | 2640 | N | PRO | 1151 | 22.255 | 25.215 | 10.846 | 1.00 | 17.40 |
| ATOM | 2641 | CD | PRO | 1151 | 20.830 | 25.499 | 11.100 | 1.00 | 15.10 |
| ATOM | 2642 | CA | PRO | 1151 | 23.133 | 26.362 | 11.130 | 1.00 | 16.16 |
| ATOM | 2643 | CB | PRO | 1151 | 22.175 | 27.520 | 11.465 | 1.00 | 14.65 |
| ATOM | 2644 | CG | PRO | 1151 | 20.870 | 26.830 | 11.842 | 1.00 | 13.77 |
| ATOM | 2645 | C | PRO | 1151 | 24.135 | 26.107 | 12.251 | 1.00 | 15.32 |
| ATOM | 2646 | O | PRO | 1151 | 23.919 | 25.249 | 13.109 | 1.00 | 15.46 |
| ATOM | 2647 | N | THR | 1152 | 25.264 | 26.775 | 12.258 | 1.00 | 14.28 |
| ATOM | 2648 | H | THR | 1152 | 25.479 | 27.400 | 11.541 | 1.00 | 0.00 |
| ATOM | 2649 | CA | THR | 1152 | 26.132 | 26.568 | 13.406 | 1.00 | 14.45 |
| ATOM | 2650 | CB | THR | 1152 | 27.577 | 26.930 | 13.142 | 1.00 | 12.68 |
| ATOM | 2651 | OG1 | THR | 1152 | 27.565 | 28.328 | 12.844 | 1.00 | 13.67 |
| ATOM | 2652 | HG1 | THR | 1152 | 28.429 | 28.571 | 12.502 | 1.00 | 0.00 |
| ATOM | 2653 | CG2 | THR | 1152 | 28.240 | 26.117 | 12.041 | 1.00 | 12.63 |
| ATOM | 2654 | C | THR | 1152 | 25.624 | 27.568 | 14.481 | 1.00 | 15.37 |
| ATOM | 2655 | O | THR | 1152 | 24.851 | 28.489 | 14.181 | 1.00 | 17.11 |
| ATOM | 2656 | N | PHE | 1153 | 26.040 | 27.491 | 15.747 | 1.00 | 15.05 |
| ATOM | 2657 | H | PHE | 1153 | 26.501 | 26.674 | 16.036 | 1.00 | 0.00 |
| ATOM | 2658 | CA | PHE | 1153 | 25.631 | 28.477 | 16.787 | 1.00 | 15.09 |
| ATOM | 2659 | CB | PHE | 1153 | 26.163 | 28.086 | 18.185 | 1.00 | 12.47 |
| ATOM | 2660 | CG | PHE | 1153 | 25.296 | 26.997 | 18.736 | 1.00 | 12.30 |
| ATOM | 2661 | CD1 | PHE | 1153 | 23.948 | 27.306 | 19.069 | 1.00 | 11.21 |
| ATOM | 2662 | CD2 | PHE | 1153 | 25.810 | 25.665 | 18.901 | 1.00 | 11.66 |
| ATOM | 2663 | CE1 | PHE | 1153 | 23.093 | 26.300 | 19.551 | 1.00 | 11.06 |
| ATOM | 2664 | CE2 | PHE | 1153 | 24.951 | 24.656 | 19.396 | 1.00 | 11.19 |
| ATOM | 2665 | CZ | PHE | 1153 | 23.607 | 24.983 | 19.720 | 1.00 | 12.62 |
| ATOM | 2666 | C | PHE | 1153 | 26.117 | 29.876 | 16.445 | 1.00 | 14.34 |
| ATOM | 2667 | O | PHE | 1153 | 25.472 | 30.840 | 16.777 | 1.00 | 14.23 |
| ATOM | 2668 | N | SER | 1154 | 27.269 | 30.071 | 15.823 | 1.00 | 13.34 |
| ATOM | 2669 | H | SER | 1154 | 27.863 | 29.307 | 15.768 | 1.00 | 0.00 |
| ATOM | 2670 | CA | SER | 1154 | 27.712 | 31.434 | 15.408 | 1.00 | 15.09 |
| ATOM | 2671 | CB | SER | 1154 | 29.096 | 31.378 | 14.785 | 1.00 | 14.24 |
| ATOM | 2672 | OG | SER | 1154 | 30.011 | 31.011 | 15.806 | 1.00 | 16.33 |
| ATOM | 2673 | HG | SER | 1154 | 30.901 | 31.092 | 15.456 | 1.00 | 0.00 |
| ATOM | 2674 | C | SER | 1154 | 26.764 | 32.104 | 14.388 | 1.00 | 13.75 |
| ATOM | 2675 | O | SER | 1154 | 26.556 | 33.316 | 14.388 | 1.00 | 13.78 |
| ATOM | 2676 | N | GLU | 1155 | 26.280 | 31.284 | 13.436 | 1.00 | 15.81 |
| ATOM | 2677 | H | GLU | 1155 | 26.604 | 30.364 | 13.443 | 1.00 | 0.00 |
| ATOM | 2678 | CA | GLU | 1155 | 25.281 | 31.682 | 12.392 | 1.00 | 16.66 |
| ATOM | 2679 | CB | GLU | 1155 | 24.987 | 30.542 | 11.377 | 1.00 | 15.31 |
| ATOM | 2680 | CG | GLU | 1155 | 26.111 | 30.385 | 10.369 | 1.00 | 22.67 |
| ATOM | 2681 | CD | GLU | 1155 | 26.092 | 29.058 | 9.587 | 1.00 | 28.28 |
| ATOM | 2682 | OE1 | GLU | 1155 | 27.089 | 28.667 | 8.954 | 1.00 | 37.04 |
| ATOM | 2683 | OE2 | GLU | 1155 | 25.098 | 28.370 | 9.612 | 1.00 | 32.99 |
| ATOM | 2684 | C | GLU | 1155 | 23.969 | 31.992 | 13.144 | 1.00 | 15.43 |
| ATOM | 2685 | O | GLU | 1155 | 23.342 | 32.994 | 12.851 | 1.00 | 14.63 |
| ATOM | 2686 | N | LEU | 1156 | 23.521 | 31.146 | 14.122 | 1.00 | 13.77 |
| ATOM | 2687 | H | LEU | 1156 | 24.025 | 30.315 | 14.261 | 1.00 | 0.00 |
| ATOM | 2688 | CA | LEU | 1156 | 22.317 | 31.399 | 14.926 | 1.00 | 11.74 |
| ATOM | 2689 | CB | LEU | 1156 | 22.098 | 30.193 | 15.887 | 1.00 | 10.53 |
| ATOM | 2690 | CG | LEU | 1156 | 21.614 | 28.925 | 15.173 | 1.00 | 7.76 |
| ATOM | 2691 | CD1 | LEU | 1156 | 21.574 | 27.666 | 16.025 | 1.00 | 8.88 |
| ATOM | 2692 | CD2 | LEU | 1156 | 20.200 | 29.205 | 14.781 | 1.00 | 11.76 |
| ATOM | 2693 | C | LEU | 1156 | 22.470 | 32.730 | 15.676 | 1.00 | 12.55 |
| ATOM | 2694 | O | LEU | 1156 | 21.562 | 33.552 | 15.687 | 1.00 | 14.12 |
| ATOM | 2695 | N | VAL | 1157 | 23.600 | 33.032 | 16.313 | 1.00 | 12.27 |
| ATOM | 2696 | H | VAL | 1157 | 24.292 | 32.340 | 16.324 | 1.00 | 0.00 |
| ATOM | 2697 | CA | VAL | 1157 | 23.871 | 34.305 | 17.042 | 1.00 | 13.32 |
| ATOM | 2698 | CB | VAL | 1157 | 25.301 | 34.219 | 17.619 | 1.00 | 11.28 |
| ATOM | 2699 | CG1 | VAL | 1157 | 25.776 | 35.623 | 18.014 | 1.00 | 11.96 |
| ATOM | 2700 | CG2 | VAL | 1157 | 25.305 | 33.194 | 18.784 | 1.00 | 9.86 |
| ATOM | 2701 | C | VAL | 1157 | 23.724 | 35.493 | 16.090 | 1.00 | 13.67 |
| ATOM | 2702 | O | VAL | 1157 | 23.065 | 36.494 | 16.345 | 1.00 | 12.94 |
| ATOM | 2703 | N | GLU | 1158 | 24.328 | 35.392 | 14.899 | 1.00 | 17.01 |
| ATOM | 2704 | H | GLU | 1158 | 24.916 | 34.624 | 14.732 | 1.00 | 0.00 |
| ATOM | 2705 | CA | GLU | 1158 | 24.192 | 36.485 | 13.889 | 1.00 | 16.82 |
| ATOM | 2706 | CB | GLU | 1158 | 25.073 | 36.156 | 12.678 | 1.00 | 17.46 |
| ATOM | 2707 | CG | GLU | 1158 | 24.934 | 37.192 | 11.506 | 1.00 | 21.95 |
| ATOM | 2708 | CD | GLU | 1158 | 25.904 | 36.914 | 10.330 | 1.00 | 22.22 |
| ATOM | 2709 | OE1 | GLU | 1158 | 26.538 | 35.851 | 10.233 | 1.00 | 23.80 |
| ATOM | 2710 | OE2 | GLU | 1158 | 25.998 | 37.784 | 9.473 | 1.00 | 25.63 |
| ATOM | 2711 | C | GLU | 1158 | 22.727 | 36.668 | 13.453 | 1.00 | 13.72 |
| ATOM | 2712 | O | GLU | 1158 | 22.156 | 37.753 | 13.527 | 1.00 | 15.36 |
| ATOM | 2713 | N | HIS | 1159 | 22.083 | 35.576 | 13.053 | 1.00 | 14.65 |
| ATOM | 2714 | H | HIS | 1159 | 22.549 | 34.714 | 13.076 | 1.00 | 0.00 |
| ATOM | 2715 | CA | HIS | 1159 | 20.679 | 35.665 | 12.619 | 1.00 | 15.40 |
| ATOM | 2716 | C | HIS | 1159 | 19.753 | 36.144 | 13.743 | 1.00 | 16.31 |
| ATOM | 2717 | O | HIS | 1159 | 18.875 | 37.008 | 13.594 | 1.00 | 15.14 |
| ATOM | 2718 | CB | HIS | 1159 | 20.239 | 34.292 | 12.118 | 1.00 | 15.70 |
| ATOM | 2719 | CG | HIS | 1159 | 18.870 | 34.362 | 11.578 | 1.00 | 21.38 |
| ATOM | 2720 | ND1 | HIS | 1159 | 17.855 | 33.518 | 11.914 | 1.00 | 24.49 |
| ATOM | 2721 | HD1 | HIS | 1159 | 17.891 | 32.742 | 12.508 | 1.00 | 0.00 |
| ATOM | 2722 | CD2 | HIS | 1159 | 18.358 | 35.214 | 10.586 | 1.00 | 21.73 |
| ATOM | 2723 | NE2 | HIS | 1159 | 17.056 | 34.887 | 10.327 | 1.00 | 20.76 |
| ATOM | 2724 | CE1 | HIS | 1159 | 16.750 | 33.847 | 11.143 | 1.00 | 23.05 |
| ATOM | 2725 | N | LEU | 1160 | 19.908 | 35.576 | 14.919 | 1.00 | 15.42 |
| ATOM | 2726 | H | LEU | 1160 | 20.546 | 34.859 | 15.015 | 1.00 | 0.00 |
| ATOM | 2727 | CA | LEU | 1160 | 19.072 | 36.005 | 16.033 | 1.00 | 17.12 |
| ATOM | 2728 | CB | LEU | 1160 | 19.254 | 35.012 | 17.291 | 1.00 | 16.70 |
| ATOM | 2729 | CG | LEU | 1160 | 18.563 | 33.622 | 17.099 | 1.00 | 14.66 |
| ATOM | 2730 | CD1 | LEU | 1160 | 18.967 | 32.662 | 18.209 | 1.00 | 14.91 |
| ATOM | 2731 | CD2 | LEU | 1160 | 17.047 | 33.835 | 17.006 | 1.00 | 16.49 |
| ATOM | 2732 | C | LEU | 1160 | 19.352 | 37.473 | 16.421 | 1.00 | 16.50 |
| ATOM | 2733 | O | LEU | 1160 | 18.438 | 38.188 | 16.861 | 1.00 | 15.92 |
| ATOM | 2734 | N | GLY | 1161 | 20.601 | 37.934 | 16.294 | 1.00 | 14.98 |
| ATOM | 2735 | H | GLY | 1161 | 21.319 | 37.313 | 16.059 | 1.00 | 0.00 |
| ATOM | 2736 | CA | GLY | 1161 | 20.912 | 39.309 | 16.605 | 1.00 | 17.59 |
| ATOM | 2737 | C | GLY | 1161 | 20.166 | 40.210 | 15.617 | 1.00 | 16.95 |
| ATOM | 2738 | O | GLY | 1161 | 19.605 | 41.234 | 15.975 | 1.00 | 17.86 |
| ATOM | 2739 | N | ASN | 1162 | 20.111 | 39.815 | 14.365 | 1.00 | 18.14 |
| ATOM | 2740 | H | ASN | 1162 | 20.623 | 39.014 | 14.135 | 1.00 | 0.00 |
| ATOM | 2741 | CA | ASN | 1162 | 19.365 | 40.550 | 13.287 | 1.00 | 17.75 |
| ATOM | 2742 | CB | ASN | 1162 | 19.473 | 39.886 | 11.932 | 1.00 | 16.47 |
| ATOM | 2743 | CG | ASN | 1162 | 20.898 | 40.081 | 11.459 | 1.00 | 17.75 |
| ATOM | 2744 | OD1 | ASN | 1162 | 21.627 | 41.031 | 11.827 | 1.00 | 18.87 |
| ATOM | 2745 | ND2 | ASN | 1162 | 21.448 | 39.230 | 10.597 | 1.00 | 20.67 |
| ATOM | 2746 | HD21 | ASN | 1162 | 21.039 | 38.434 | 10.212 | 1.00 | 0.00 |
| ATOM | 2747 | HD22 | ASN | 1162 | 22.384 | 39.458 | 10.358 | 1.00 | 0.00 |
| ATOM | 2748 | C | ASN | 1162 | 17.876 | 40.583 | 13.582 | 1.00 | 19.00 |
| ATOM | 2749 | O | ASN | 1162 | 17.164 | 41.581 | 13.444 | 1.00 | 20.58 |
| ATOM | 2750 | N | LEU | 1163 | 17.331 | 39.419 | 13.909 | 1.00 | 18.57 |
| ATOM | 2751 | H | LEU | 1163 | 17.898 | 38.623 | 13.934 | 1.00 | 0.00 |
| ATOM | 2752 | CA | LEU | 1163 | 15.908 | 39.324 | 14.236 | 1.00 | 19.25 |
| ATOM | 2753 | CB | LEU | 1163 | 15.590 | 37.852 | 14.505 | 1.00 | 22.44 |
| ATOM | 2754 | CG | LEU | 1163 | 14.868 | 36.976 | 13.481 | 1.00 | 21.63 |
| ATOM | 2755 | CD1 | LEU | 1163 | 15.377 | 37.272 | 12.094 | 1.00 | 23.84 |
| ATOM | 2756 | CD2 | LEU | 1163 | 15.025 | 35.508 | 13.888 | 1.00 | 24.24 |
| ATOM | 2757 | C | LEU | 1163 | 15.613 | 40.191 | 15.453 | 1.00 | 22.69 |
| ATOM | 2758 | O | LEU | 1163 | 14.567 | 40.800 | 15.580 | 1.00 | 24.43 |
| ATOM | 2759 | N | LEU | 1164 | 16.475 | 40.248 | 16.452 | 1.00 | 25.66 |
| ATOM | 2760 | H | LEU | 1164 | 17.276 | 39.689 | 16.404 | 1.00 | 0.00 |
| ATOM | 2761 | CA | LEU | 1164 | 16.248 | 41.105 | 17.646 | 1.00 | 26.60 |
| ATOM | 2762 | CB | LEU | 1164 | 17.349 | 40.797 | 18.641 | 1.00 | 27.36 |
| ATOM | 2763 | CG | LEU | 1164 | 17.224 | 41.257 | 20.072 | 1.00 | 28.08 |
| ATOM | 2764 | CD1 | LEU | 1164 | 15.843 | 40.984 | 20.728 | 1.00 | 25.82 |
| ATOM | 2765 | CD2 | LEU | 1164 | 18.360 | 40.470 | 20.784 | 1.00 | 30.50 |
| ATOM | 2766 | C | LEU | 1164 | 16.246 | 42.579 | 17.264 | 1.00 | 29.32 |
| ATOM | 2767 | O | LEU | 1164 | 15.390 | 43.362 | 17.677 | 1.00 | 30.29 |
| ATOM | 2768 | N | GLN | 1165 | 17.222 | 42.940 | 16.445 | 1.00 | 30.62 |
| ATOM | 2769 | H | GLN | 1165 | 17.874 | 42.269 | 16.170 | 1.00 | 0.00 |
| ATOM | 2770 | CA | GLN | 1165 | 17.346 | 44.298 | 15.964 | 1.00 | 33.29 |
| ATOM | 2771 | CB | GLN | 1165 | 18.531 | 44.311 | 14.989 | 1.00 | 34.21 |
| ATOM | 2772 | CG | GLN | 1165 | 19.188 | 45.698 | 14.910 | 1.00 | 41.96 |
| ATOM | 2773 | CD | GLN | 1165 | 18.623 | 46.807 | 15.844 | 1.00 | 46.67 |
| ATOM | 2774 | OE1 | GLN | 1165 | 18.720 | 46.843 | 17.076 | 1.00 | 48.88 |
| ATOM | 2775 | NE2 | GLN | 1165 | 17.981 | 47.828 | 15.297 | 1.00 | 47.75 |
| ATOM | 2776 | HE21 | GLN | 1165 | 17.852 | 47.959 | 14.344 | 1.00 | 0.00 |
| ATOM | 2777 | HE22 | GLN | 1165 | 17.656 | 48.469 | 15.960 | 1.00 | 0.00 |
| ATOM | 2778 | C | GLN | 1165 | 16.021 | 44.716 | 15.328 | 1.00 | 34.48 |
| ATOM | 2779 | O | GLN | 1165 | 15.549 | 45.835 | 15.561 | 1.00 | 35.45 |
| ATOM | 2780 | N | ALA | 1166 | 15.466 | 43.856 | 14.462 | 1.00 | 35.26 |
| ATOM | 2781 | H | ALA | 1166 | 15.890 | 42.995 | 14.297 | 1.00 | 0.00 |
| ATOM | 2782 | CA | ALA | 1166 | 14.140 | 44.113 | 13.871 | 1.00 | 35.85 |
| ATOM | 2783 | CB | ALA | 1166 | 13.887 | 43.060 | 12.755 | 1.00 | 33.48 |
| ATOM | 2784 | C | ALA | 1166 | 12.803 | 44.224 | 14.696 | 1.00 | 38.95 |
| ATOM | 2785 | O | ALA | 1166 | 12.510 | 43.591 | 15.762 | 1.00 | 37.54 |
| ATOM | 2786 | C01 | INH | 1 | 15.467 | 29.112 | 41.672 | 1.00 | 16.51 |
| ATOM | 2787 | C02 | INH | 1 | 15.685 | 27.766 | 41.350 | 1.00 | 15.34 |
| ATOM | 2788 | N03 | INH | 1 | 14.655 | 26.875 | 41.373 | 1.00 | 18.13 |
| ATOM | 2789 | C04 | INH | 1 | 13.401 | 27.213 | 41.702 | 1.00 | 19.07 |
| ATOM | 2790 | C05 | INH | 1 | 13.114 | 28.553 | 42.038 | 1.00 | 18.86 |
| ATOM | 2791 | C06 | INH | 1 | 14.162 | 29.502 | 42.019 | 1.00 | 17.99 |
| ATOM | 2792 | C07 | INH | 1 | 21.520 | 24.536 | 38.589 | 1.00 | 12.62 |
| ATOM | 2793 | C08 | INH | 1 | 20.160 | 24.260 | 38.834 | 1.00 | 11.94 |
| ATOM | 2794 | C09 | INH | 1 | 19.325 | 25.237 | 39.427 | 1.00 | 15.33 |
| ATOM | 2795 | C10 | INH | 1 | 19.855 | 26.515 | 39.732 | 1.00 | 15.23 |
| ATOM | 2796 | C11 | INH | 1 | 21.243 | 26.769 | 39.493 | 1.00 | 12.06 |
| ATOM | 2797 | C12 | INH | 1 | 22.068 | 25.798 | 38.917 | 1.00 | 13.31 |
| ATOM | 2798 | C13 | INH | 1 | 19.352 | 27.721 | 40.311 | 1.00 | 13.65 |
| ATOM | 2799 | N14 | INH | 1 | 20.344 | 28.621 | 40.400 | 1.00 | 11.82 |
| ATOM | 2800 | N15 | INH | 1 | 21.499 | 28.029 | 39.877 | 1.00 | 11.69 |
| ATOM | 2801 | C16 | INH | 1 | 17.995 | 28.165 | 40.726 | 1.00 | 11.01 |
| ATOM | 2802 | C17 | INH | 1 | 16.993 | 27.309 | 40.792 | 1.00 | 12.96 |
| ATOM | 2803 | C18 | INH | 1 | 24.002 | 23.389 | 38.671 | 1.00 | 9.96 |
| ATOM | 2804 | C19 | INH | 1 | 23.960 | 23.670 | 40.050 | 1.00 | 12.52 |
| ATOM | 2805 | C20 | INH | 1 | 25.061 | 23.588 | 40.873 | 1.00 | 9.20 |
| ATOM | 2806 | C21 | INH | 1 | 26.277 | 23.249 | 40.288 | 1.00 | 8.09 |
| ATOM | 2807 | C22 | INH | 1 | 26.358 | 22.982 | 38.922 | 1.00 | 12.01 |
| ATOM | 2808 | C23 | INH | 1 | 25.231 | 23.078 | 38.083 | 1.00 | 9.75 |
| ATOM | 2809 | S24 | INH | 1 | 22.477 | 23.349 | 37.768 | 1.00 | 14.45 |
| ATOM | 2810 | H25 | INH | 1 | 22.370 | 28.468 | 39.825 | 1.00 | 0.00 |
| ATOM | 2813 | C26 | INH | 1 | 25.459 | 22.914 | 36.652 | 1.00 | 10.95 |
| ATOM | 2814 | O27 | INH | 1 | 24.820 | 23.486 | 35.793 | 1.00 | 9.95 |
| ATOM | 2815 | N28 | INH | 1 | 26.375 | 22.067 | 36.237 | 1.00 | 8.18 |
| ATOM | 2816 | C29 | INH | 1 | 26.728 | 21.827 | 34.836 | 1.00 | 11.21 |
| ATOM | 2817 | H30 | INH | 1 | 26.878 | 21.621 | 36.929 | 1.00 | 0.00 |
| ATOM | 2818 | OH2 | WAT | 2 | 21.558 | 15.543 | 31.347 | 1.00 | 34.57 |
| ATOM | 2821 | OH2 | WAT | 3 | 19.748 | 10.969 | 30.910 | 1.00 | 34.58 |
| ATOM | 2824 | OH2 | WAT | 4 | 21.365 | 12.094 | 34.955 | 1.00 | 21.09 |
| ATOM | 2827 | OH2 | WAT | 5 | 19.451 | 12.246 | 36.658 | 1.00 | 33.66 |
| ATOM | 2830 | OH2 | WAT | 6 | 20.409 | 11.131 | 40.399 | 1.00 | 19.44 |
| ATOM | 2833 | OH2 | WAT | 7 | 36.609 | 23.110 | 51.197 | 1.00 | 16.49 |
| ATOM | 2836 | OH2 | WAT | 8 | 41.839 | 23.999 | 37.833 | 1.00 | 21.62 |
| ATOM | 2839 | OH2 | WAT | 9 | 28.639 | 15.176 | 47.260 | 1.00 | 17.72 |
| ATOM | 2842 | OH2 | WAT | 10 | 30.568 | 3.384 | 45.911 | 1.00 | 16.97 |
| ATOM | 2845 | OH2 | WAT | 11 | 35.989 | 6.449 | 36.040 | 1.00 | 23.89 |
| ATOM | 2848 | OH2 | WAT | 12 | 37.523 | 13.529 | 37.453 | 1.00 | 33.78 |
| ATOM | 2851 | OH2 | WAT | 13 | 12.478 | 21.488 | 28.850 | 1.00 | 13.54 |
| ATOM | 2854 | OH2 | WAT | 14 | 8.838 | 23.808 | 27.621 | 1.00 | 15.58 |
| ATOM | 2857 | OH2 | WAT | 15 | 7.642 | 15.080 | 27.836 | 1.00 | 28.94 |
| ATOM | 2860 | OH2 | WAT | 16 | 11.342 | 8.718 | 32.654 | 1.00 | 33.92 |
| ATOM | 2863 | OH2 | WAT | 17 | 9.649 | 5.659 | 29.783 | 1.00 | 44.22 |
| ATOM | 2866 | OH2 | WAT | 18 | −2.485 | 28.559 | 27.818 | 1.00 | 18.39 |
| ATOM | 2869 | OH2 | WAT | 19 | −1.777 | 37.362 | 28.183 | 1.00 | 28.44 |
| ATOM | 2872 | OH2 | WAT | 20 | 14.649 | 38.739 | 34.763 | 1.00 | 16.67 |
| ATOM | 2875 | OH2 | WAT | 21 | 8.109 | 42.802 | 21.661 | 1.00 | 27.19 |
| ATOM | 2878 | OH2 | WAT | 22 | 3.134 | 25.196 | 11.045 | 1.00 | 21.51 |
| ATOM | 2881 | OH2 | WAT | 23 | 9.971 | 21.846 | 32.653 | 1.00 | 34.42 |
| ATOM | 2884 | OH2 | WAT | 24 | 14.433 | 32.599 | 9.687 | 1.00 | 17.36 |
| ATOM | 2887 | OH2 | WAT | 25 | 11.398 | 37.195 | 14.059 | 1.00 | 26.11 |
| ATOM | 2890 | OH2 | WAT | 26 | 29.781 | 37.244 | 28.518 | 1.00 | 21.43 |
| ATOM | 2893 | OH2 | WAT | 27 | 35.849 | 33.325 | 30.099 | 1.00 | 20.27 |
| ATOM | 2896 | OH2 | WAT | 28 | 27.464 | 25.058 | 16.086 | 1.00 | 20.82 |
| ATOM | 2899 | OH2 | WAT | 29 | 29.392 | 28.058 | 16.329 | 1.00 | 19.43 |
| ATOM | 2902 | OH2 | WAT | 30 | 23.910 | 16.638 | 21.546 | 1.00 | 21.47 |
| ATOM | 2905 | OH2 | WAT | 31 | 19.224 | 16.063 | 23.953 | 1.00 | 13.66 |
| ATOM | 2908 | OH2 | WAT | 32 | 20.976 | 7.537 | 21.855 | 1.00 | 36.76 |
| ATOM | 2911 | OH2 | WAT | 33 | 25.809 | 9.009 | 12.082 | 1.00 | 48.71 |
| ATOM | 2914 | OH2 | WAT | 34 | 15.566 | 22.277 | 7.342 | 1.00 | 24.82 |
| ATOM | 2917 | OH2 | WAT | 35 | 30.692 | 13.466 | 47.582 | 1.00 | 10.58 |
| ATOM | 2920 | OH2 | WAT | 36 | 36.346 | 29.066 | 57.836 | 1.00 | 32.66 |
| ATOM | 2923 | OH2 | WAT | 37 | 38.598 | 27.305 | 56.608 | 1.00 | 31.42 |
| ATOM | 2926 | OH2 | WAT | 38 | 13.255 | 20.852 | 9.351 | 1.00 | 19.38 |
| ATOM | 2929 | OH2 | WAT | 39 | 9.204 | 44.310 | 24.552 | 1.00 | 51.64 |
| ATOM | 2932 | OH2 | WAT | 40 | 10.488 | 20.416 | 30.249 | 1.00 | 20.12 |
| ATOM | 2935 | OH2 | WAT | 41 | 1.444 | 22.307 | 27.486 | 1.00 | 20.16 |
| ATOM | 2938 | OH2 | WAT | 42 | 13.644 | 13.819 | 18.526 | 1.00 | 18.27 |
| ATOM | 2941 | OH2 | WAT | 43 | 20.807 | 15.124 | 19.891 | 1.00 | 27.83 |
| ATOM | 2944 | OH2 | WAT | 44 | 28.744 | 18.128 | 13.980 | 1.00 | 42.03 |
| ATOM | 2947 | OH2 | WAT | 45 | 14.812 | 13.877 | 8.917 | 1.00 | 42.90 |
| ATOM | 2950 | OH2 | WAT | 46 | 3.552 | 17.624 | 17.177 | 1.00 | 24.34 |
| ATOM | 2953 | OH2 | WAT | 47 | 2.345 | 26.652 | 20.940 | 1.00 | 12.63 |
| ATOM | 2956 | OH2 | WAT | 48 | 32.232 | 29.072 | 34.806 | 1.00 | 34.28 |
| ATOM | 2959 | OH2 | WAT | 49 | 33.494 | 30.874 | 36.450 | 1.00 | 35.88 |
| ATOM | 2962 | OH2 | WAT | 50 | 37.104 | 30.711 | 23.619 | 1.00 | 42.33 |
| ATOM | 2965 | OH2 | WAT | 51 | 42.763 | 27.358 | 26.526 | 1.00 | 27.63 |
| ATOM | 2968 | OH2 | WAT | 52 | 39.763 | 21.647 | 34.543 | 1.00 | 15.62 |
| ATOM | 2971 | OH2 | WAT | 53 | 20.360 | 12.990 | 32.658 | 1.00 | 23.90 |
| ATOM | 2974 | OH2 | WAT | 54 | 21.438 | 9.014 | 31.865 | 1.00 | 61.41 |
| ATOM | 2977 | OH2 | WAT | 55 | 15.153 | 18.427 | 39.325 | 1.00 | 25.59 |
| ATOM | 2980 | OH2 | WAT | 56 | 19.887 | 37.242 | 9.240 | 1.00 | 26.73 |
| ATOM | 2983 | OH2 | WAT | 57 | 11.742 | 12.959 | 25.389 | 1.00 | 19.57 |
| ATOM | 2986 | OH2 | WAT | 58 | 37.896 | 28.978 | 34.608 | 1.00 | 20.00 |
| ATOM | 2989 | OH2 | WAT | 59 | 38.225 | 32.550 | 36.300 | 1.00 | 24.86 |
| ATOM | 2992 | OH2 | WAT | 60 | 40.920 | 28.730 | 47.779 | 1.00 | 40.96 |
| ATOM | 2995 | OH2 | WAT | 61 | 41.266 | 28.151 | 33.491 | 1.00 | 51.77 |
| ATOM | 2998 | OH2 | WAT | 62 | 22.799 | 9.655 | 34.661 | 1.00 | 36.76 |
| ATOM | 3001 | OH2 | WAT | 63 | 21.634 | 13.436 | 26.930 | 1.00 | 31.52 |
| ATOM | 3004 | OH2 | WAT | 64 | 13.507 | 15.777 | 38.782 | 1.00 | 31.16 |
| ATOM | 3007 | OH2 | WAT | 65 | 11.587 | 21.132 | 34.646 | 1.00 | 21.10 |
| ATOM | 3010 | OH2 | WAT | 66 | 20.242 | 12.417 | 51.381 | 1.00 | 31.88 |
| ATOM | 3013 | OH2 | WAT | 67 | 24.611 | 4.098 | 40.864 | 1.00 | 29.27 |
| ATOM | 3016 | OH2 | WAT | 68 | 22.783 | 37.150 | 46.811 | 1.00 | 20.27 |
| ATOM | 3019 | OH2 | WAT | 69 | 29.339 | 33.603 | 22.286 | 1.00 | 19.25 |
| ATOM | 3022 | OH2 | WAT | 70 | 31.445 | 28.598 | 18.482 | 1.00 | 16.25 |
| ATOM | 3025 | OH2 | WAT | 71 | 30.105 | 25.576 | 16.144 | 1.00 | 30.46 |
| ATOM | 3028 | OH2 | WAT | 72 | 4.652 | 8.996 | 25.021 | 1.00 | 48.28 |
| ATOM | 3031 | OH2 | WAT | 73 | 8.228 | 20.711 | 28.704 | 1.00 | 30.27 |
| ATOM | 3034 | OH2 | WAT | 74 | 4.732 | 27.221 | 37.992 | 1.00 | 39.68 |
| ATOM | 3037 | OH2 | WAT | 75 | 4.619 | 26.584 | 35.351 | 1.00 | 26.52 |
| ATOM | 3040 | OH2 | WAT | 76 | −4.638 | 27.064 | 27.962 | 1.00 | 11.09 |
| ATOM | 3043 | OH2 | WAT | 77 | 14.851 | 41.435 | 37.319 | 1.00 | 33.55 |
| ATOM | 3046 | OH2 | WAT | 78 | 15.763 | 30.849 | 8.093 | 1.00 | 20.24 |
| ATOM | 3049 | OH2 | WAT | 79 | 16.483 | 28.248 | 5.360 | 1.00 | 33.46 |
| ATOM | 3052 | OH2 | WAT | 80 | 20.689 | 27.490 | 7.873 | 1.00 | 28.53 |
| ATOM | 3055 | OH2 | WAT | 81 | 19.269 | 32.602 | 7.780 | 1.00 | 39.83 |
| ATOM | 3058 | OH2 | WAT | 82 | 36.878 | 30.725 | 29.104 | 1.00 | 25.35 |
| ATOM | 3061 | OH2 | WAT | 83 | 21.344 | 16.605 | 21.979 | 1.00 | 32.91 |
| ATOM | 3064 | OH2 | WAT | 84 | 17.778 | 17.607 | 25.626 | 1.00 | 10.15 |
| ATOM | 3067 | OH2 | WAT | 85 | 21.615 | 19.013 | 23.652 | 1.00 | 16.81 |
| ATOM | 3070 | OH2 | WAT | 86 | 27.083 | 13.777 | 13.076 | 1.00 | 23.62 |
| ATOM | 3073 | OH2 | WAT | 87 | 25.475 | 11.817 | 11.606 | 1.00 | 45.34 |
| ATOM | 3076 | OH2 | WAT | 88 | 6.667 | 22.239 | 8.919 | 1.00 | 47.37 |
| ATOM | 3079 | OH2 | WAT | 89 | 2.330 | 33.344 | 16.188 | 1.00 | 43.32 |
| ATOM | 3082 | OH2 | WAT | 90 | 29.021 | 23.106 | 10.650 | 1.00 | 49.29 |
| ATOM | 3085 | OH2 | WAT | 91 | 30.893 | 18.696 | 17.687 | 1.00 | 32.91 |
| ATOM | 3088 | OH2 | WAT | 92 | 32.465 | 16.031 | 18.003 | 1.00 | 39.83 |
| ATOM | 3091 | OH2 | WAT | 93 | 23.477 | 33.842 | 9.983 | 1.00 | 22.25 |
| ATOM | 3094 | OH2 | WAT | 94 | −7.628 | 36.661 | 32.110 | 1.00 | 27.53 |
| ATOM | 3097 | OH2 | WAT | 95 | −6.707 | 37.614 | 34.593 | 1.00 | 35.91 |
| ATOM | 3100 | OH2 | WAT | 96 | 1.245 | 22.504 | 22.273 | 1.00 | 23.01 |
| ATOM | 3103 | OH2 | WAT | 97 | 5.312 | 13.742 | 25.671 | 1.00 | 42.58 |
| ATOM | 3106 | OH2 | WAT | 98 | 8.971 | 12.439 | 26.644 | 1.00 | 18.65 |
| ATOM | 3109 | OH2 | WAT | 99 | 20.411 | 3.568 | 25.535 | 1.00 | 35.05 |
| ATOM | 3112 | OH2 | WAT | 100 | 20.415 | 12.730 | 11.212 | 1.00 | 30.79 |
| ATOM | 3115 | OH2 | WAT | 101 | 9.622 | 40.764 | 32.530 | 1.00 | 43.44 |
| ATOM | 3118 | OH2 | WAT | 102 | 14.210 | 21.833 | 35.197 | 1.00 | 29.17 |
| ATOM | 3121 | OH2 | WAT | 103 | 11.160 | 18.116 | 33.043 | 1.00 | 40.76 |
| ATOM | 3124 | OH2 | WAT | 104 | 7.861 | 18.112 | 28.063 | 1.00 | 23.43 |
| ATOM | 3127 | OH2 | WAT | 105 | 7.046 | 22.298 | 36.473 | 1.00 | 41.52 |
| ATOM | 3130 | OH2 | WAT | 106 | 12.514 | 17.198 | 36.511 | 1.00 | 35.64 |
| ATOM | 3133 | OH2 | WAT | 107 | 17.009 | 28.028 | 51.290 | 1.00 | 23.77 |
| ATOM | 3136 | OH2 | WAT | 108 | 20.765 | 29.092 | 53.372 | 1.00 | 32.30 |
| ATOM | 3139 | OH2 | WAT | 109 | 24.557 | 24.300 | 56.556 | 1.00 | 33.15 |
| ATOM | 3142 | OH2 | WAT | 110 | 35.271 | 33.661 | 52.591 | 1.00 | 24.90 |
| ATOM | 3145 | OH2 | WAT | 111 | 30.643 | 36.640 | 47.962 | 1.00 | 27.93 |
| ATOM | 3148 | OH2 | WAT | 112 | 35.933 | 41.719 | 46.758 | 1.00 | 38.16 |
| ATOM | 3151 | OH2 | WAT | 113 | 28.319 | 39.907 | 54.314 | 1.00 | 28.23 |
| ATOM | 3154 | OH2 | WAT | 114 | 24.690 | 41.190 | 53.704 | 1.00 | 35.42 |
| ATOM | 3157 | OH2 | WAT | 115 | 25.486 | 40.639 | 51.219 | 1.00 | 31.03 |
| ATOM | 3160 | OH2 | WAT | 116 | 23.100 | 34.896 | 38.725 | 1.00 | 42.37 |
| ATOM | 3163 | OH2 | WAT | 117 | 10.823 | 38.831 | 33.837 | 1.00 | 49.21 |
| ATOM | 3166 | OH2 | WAT | 118 | 7.690 | 37.458 | 36.305 | 1.00 | 47.42 |
| ATOM | 3169 | OH2 | WAT | 119 | 5.799 | 33.794 | 37.318 | 1.00 | 28.21 |
| ATOM | 3172 | OH2 | WAT | 120 | 2.368 | 20.697 | 24.107 | 1.00 | 19.92 |
| ATOM | 3175 | OH2 | WAT | 121 | 0.378 | 24.277 | 24.154 | 1.00 | 25.89 |
| ATOM | 3178 | OH2 | WAT | 122 | 7.722 | 30.497 | 8.738 | 1.00 | 25.68 |
| ATOM | 3181 | OH2 | WAT | 123 | 13.365 | 24.198 | 6.019 | 1.00 | 35.59 |
| ATOM | 3184 | OH2 | WAT | 124 | 17.075 | 24.349 | 6.476 | 1.00 | 39.47 |
| ATOM | 3187 | OH2 | WAT | 125 | 15.779 | 17.507 | 6.275 | 1.00 | 36.83 |
| ATOM | 3190 | OH2 | WAT | 126 | 27.470 | 15.077 | 8.844 | 1.00 | 42.62 |
| ATOM | 3193 | OH2 | WAT | 127 | 29.691 | 22.883 | 13.565 | 1.00 | 42.23 |
| ATOM | 3196 | OH2 | WAT | 128 | 32.124 | 32.887 | 22.257 | 1.00 | 28.43 |
| ATOM | 3199 | OH2 | WAT | 129 | 29.375 | 36.312 | 23.573 | 1.00 | 27.73 |
| ATOM | 3202 | OH2 | WAT | 130 | 41.067 | 23.537 | 32.910 | 1.00 | 23.39 |
| ATOM | 3205 | OH2 | WAT | 131 | 43.927 | 28.077 | 37.972 | 1.00 | 31.50 |
| ATOM | 3208 | OH2 | WAT | 132 | 44.964 | 30.901 | 38.738 | 1.00 | 37.57 |
| ATOM | 3211 | OH2 | WAT | 133 | 35.799 | 22.625 | 53.733 | 1.00 | 39.11 |
| ATOM | 3214 | OH2 | WAT | 134 | 19.839 | 9.456 | 38.121 | 1.00 | 37.70 |
| ATOM | 3217 | OH2 | WAT | 135 | 20.489 | 5.751 | 36.729 | 1.00 | 47.67 |
| ATOM | 3220 | OH2 | WAT | 136 | 24.240 | 7.520 | 37.753 | 1.00 | 37.13 |
| ATOM | 3223 | OH2 | WAT | 137 | 24.010 | 13.717 | 34.742 | 1.00 | 40.20 |
| ATOM | 3226 | OH2 | WAT | 138 | 30.098 | 14.460 | 34.736 | 1.00 | 24.45 |
| ATOM | 3229 | OH2 | WAT | 139 | 31.427 | 7.867 | 36.965 | 1.00 | 21.00 |
| ATOM | 3232 | OH2 | WAT | 140 | 32.907 | 9.795 | 35.276 | 1.00 | 51.73 |
| ATOM | 3235 | OH2 | WAT | 141 | 35.344 | 7.757 | 32.156 | 1.00 | 32.83 |
| ATOM | 3238 | OH2 | WAT | 142 | 31.384 | 11.635 | 33.682 | 1.00 | 38.01 |
| ATOM | 3241 | OH2 | WAT | 143 | 23.343 | 3.443 | 43.671 | 1.00 | 35.01 |
| ATOM | 3244 | OH2 | WAT | 144 | 18.659 | 6.471 | 45.608 | 1.00 | 31.79 |
| ATOM | 3247 | OH2 | WAT | 145 | 19.341 | 15.404 | 33.982 | 1.00 | 18.01 |
| ATOM | 3250 | OH2 | WAT | 146 | 27.139 | 8.710 | 37.331 | 1.00 | 29.64 |
| ATOM | 3253 | OH2 | WAT | 147 | 31.256 | 3.321 | 42.556 | 1.00 | 31.65 |
| ATOM | 3256 | OH2 | WAT | 148 | 28.359 | 2.450 | 41.120 | 1.00 | 32.66 |
| ATOM | 3259 | OH2 | WAT | 149 | 31.314 | 27.256 | 32.926 | 1.00 | 40.37 |
| ATOM | 3262 | OH2 | WAT | 150 | 36.263 | 30.965 | 35.112 | 1.00 | 24.03 |
| ATOM | 3265 | OH2 | WAT | 151 | 37.239 | 35.994 | 29.752 | 1.00 | 38.16 |
| ATOM | 3268 | OH2 | WAT | 152 | 39.250 | 35.575 | 27.849 | 1.00 | 42.33 |
| ATOM | 3271 | OH2 | WAT | 153 | 36.640 | 32.308 | 26.467 | 1.00 | 52.84 |
| ATOM | 3274 | OH2 | WAT | 154 | 37.255 | 9.810 | 37.726 | 1.00 | 43.76 |
| ATOM | 3277 | OH2 | WAT | 155 | 37.286 | 11.517 | 40.837 | 1.00 | 42.03 |
| ATOM | 3280 | OH2 | WAT | 156 | 43.304 | 18.445 | 42.509 | 1.00 | 40.18 |
| ATOM | 3283 | OH2 | WAT | 157 | 34.361 | 32.941 | 43.024 | 1.00 | 30.66 |
| ATOM | 3286 | OH2 | WAT | 158 | 35.844 | 29.928 | 45.687 | 1.00 | 25.74 |
| ATOM | 3289 | OH2 | WAT | 159 | 1.268 | 29.676 | 15.117 | 1.00 | 47.31 |
| ATOM | 3292 | OH2 | WAT | 160 | 34.609 | 13.989 | 49.448 | 1.00 | 20.17 |
| ATOM | 3295 | OH2 | WAT | 161 | 35.958 | 15.397 | 47.533 | 1.00 | 30.96 |
| ATOM | 3298 | OH2 | WAT | 162 | 40.889 | 17.103 | 44.440 | 1.00 | 48.86 |
| ATOM | 3301 | OH2 | WAT | 163 | 35.013 | 24.475 | 56.536 | 1.00 | 26.72 |
| ATOM | 3304 | OH2 | WAT | 164 | 28.024 | 23.574 | 54.776 | 1.00 | 39.22 |
| ATOM | 3307 | OH2 | WAT | 165 | 25.222 | 7.144 | 49.428 | 1.00 | 24.76 |
| ATOM | 3310 | OH2 | WAT | 166 | 25.601 | 9.166 | 51.153 | 1.00 | 34.45 |
| ATOM | 3313 | OH2 | WAT | 167 | 18.489 | 35.617 | 47.784 | 1.00 | 44.87 |
| ATOM | 3316 | OH2 | WAT | 168 | 22.476 | 37.926 | 51.045 | 1.00 | 60.03 |
| ATOM | 3319 | OH2 | WAT | 169 | 22.322 | 35.747 | 44.073 | 1.00 | 46.42 |
| ATOM | 3322 | OH2 | WAT | 170 | 23.612 | 36.162 | 41.598 | 1.00 | 42.59 |
| ATOM | 3325 | OH2 | WAT | 171 | 14.381 | 20.419 | 37.568 | 1.00 | 46.86 |
| ATOM | 3328 | OH2 | WAT | 172 | 22.761 | 3.445 | 38.697 | 1.00 | 49.49 |
| ATOM | 3331 | OH2 | WAT | 173 | 33.241 | 5.858 | 37.182 | 1.00 | 28.92 |
| ATOM | 3334 | OH2 | WAT | 174 | 34.965 | 2.244 | 43.799 | 1.00 | 44.57 |
| ATOM | 3337 | OH2 | WAT | 175 | 34.333 | 43.886 | 49.298 | 1.00 | 46.97 |
| ATOM | 3340 | OH2 | WAT | 176 | 25.565 | 0.658 | 41.945 | 1.00 | 47.19 |
| ATOM | 3343 | OH2 | WAT | 177 | 26.313 | 11.651 | 35.719 | 1.00 | 31.94 |
| ATOM | 3346 | OH2 | WAT | 178 | 32.504 | 24.467 | 16.970 | 1.00 | 50.42 |
| ATOM | 3349 | OH2 | WAT | 179 | 34.156 | 27.484 | 17.903 | 1.00 | 48.54 |
| ATOM | 3352 | OH2 | WAT | 180 | 8.001 | 18.060 | 32.756 | 1.00 | 51.39 |
| ATOM | 3355 | OH2 | WAT | 181 | 0.047 | 33.978 | 33.580 | 1.00 | 21.19 |
| ATOM | 3358 | OH2 | WAT | 182 | 32.279 | 36.464 | 20.504 | 1.00 | 54.71 |
| ATOM | 3361 | OH2 | WAT | 183 | 42.067 | 30.423 | 24.561 | 1.00 | 43.67 |
| ATOM | 3364 | OH2 | WAT | 184 | 20.740 | 13.907 | 24.216 | 1.00 | 50.83 |
| ATOM | 3367 | OH2 | WAT | 185 | 19.436 | 35.382 | 40.597 | 1.00 | 24.45 |
| ATOM | 3370 | OH2 | WAT | 186 | 20.417 | 13.148 | 29.352 | 1.00 | 53.87 |
| ATOM | 3373 | OH2 | WAT | 187 | 13.036 | 16.554 | 18.143 | 1.00 | 16.99 |
| ATOM | 3376 | OH2 | WAT | 188 | 10.226 | 18.910 | 15.071 | 1.00 | 15.64 |
| ATOM | 3379 | OH2 | WAT | 189 | 7.822 | 12.284 | 14.165 | 1.00 | 32.90 |
| ATOM | 3382 | OH2 | WAT | 190 | 3.971 | 14.915 | 8.760 | 1.00 | 56.19 |
| ATOM | 3385 | OH2 | WAT | 191 | 5.965 | 17.594 | 9.360 | 1.00 | 40.85 |
| ATOM | 3388 | OH2 | WAT | 192 | 11.199 | 20.596 | 7.314 | 1.00 | 37.09 |
| ATOM | 3391 | OH2 | WAT | 193 | 3.315 | 16.674 | 24.185 | 1.00 | 37.23 |
| ATOM | 3394 | OH2 | WAT | 194 | 4.561 | 13.987 | 23.164 | 1.00 | 30.44 |
| ATOM | 3397 | OH2 | WAT | 195 | 4.453 | 7.658 | 17.424 | 1.00 | 40.88 |
| ATOM | 3400 | OH2 | WAT | 196 | 25.666 | 15.105 | 15.045 | 1.00 | 29.64 |
| ATOM | 3403 | OH2 | WAT | 197 | 23.330 | 12.169 | 9.822 | 1.00 | 41.58 |
| ATOM | 3406 | OH2 | WAT | 198 | 20.744 | 4.272 | 11.486 | 1.00 | 38.45 |
| ATOM | 3409 | OH2 | WAT | 199 | 29.873 | 12.613 | 14.097 | 1.00 | 49.09 |
| ATOM | 3412 | OH2 | WAT | 200 | 19.695 | 24.896 | 7.766 | 1.00 | 29.94 |
| ATOM | 3415 | OH2 | WAT | 201 | 22.211 | 35.676 | 8.496 | 1.00 | 38.59 |
| ATOM | 3418 | OH2 | WAT | 202 | 23.806 | 39.490 | 8.814 | 1.00 | 30.32 |
| ATOM | 3421 | OH2 | WAT | 203 | 8.784 | 38.483 | 40.768 | 1.00 | 56.25 |
| ATOM | 3424 | OH2 | WAT | 204 | 0.692 | 26.651 | 30.993 | 1.00 | 22.58 |
| ATOM | 3427 | OH2 | WAT | 205 | −0.855 | 24.992 | 29.691 | 1.00 | 34.48 |
| ATOM | 3430 | OH2 | WAT | 206 | 32.745 | 30.270 | 14.656 | 1.00 | 59.01 |
| ATOM | 3433 | OH2 | WAT | 207 | 39.955 | 23.802 | 24.361 | 1.00 | 48.87 |
| ATOM | 3436 | OH2 | WAT | 208 | 39.092 | 25.709 | 22.524 | 1.00 | 41.28 |
| ATOM | 3439 | OH2 | WAT | 209 | 38.120 | 27.793 | 20.132 | 1.00 | 37.51 |
| ATOM | 3442 | OH2 | WAT | 210 | 35.318 | 12.365 | 31.086 | 1.00 | 46.78 |
| ATOM | 3445 | OH2 | WAT | 211 | 27.309 | 36.193 | 35.134 | 1.00 | 26.64 |
| ATOM | 3448 | OH2 | WAT | 212 | 3.494 | 20.818 | 16.711 | 1.00 | 31.85 |
| ATOM | 3451 | OH2 | WAT | 213 | 28.303 | 39.213 | 45.416 | 1.00 | 37.98 |
| ATOM | 3454 | OH2 | WAT | 214 | 26.692 | 20.777 | 4.305 | 1.00 | 44.85 |
| ATOM | 3457 | OH2 | WAT | 215 | 16.690 | 6.182 | 35.698 | 1.00 | 44.81 |
| ATOM | 3460 | OH2 | WAT | 216 | 6.380 | 29.513 | 38.647 | 1.00 | 41.88 |
| ATOM | 3463 | OH2 | WAT | 217 | 14.971 | 24.510 | 40.178 | 1.00 | 26.95 |
| ATOM | 3466 | OH2 | WAT | 218 | 13.404 | 25.557 | 38.397 | 1.00 | 19.80 |
| ATOM | 3469 | OH2 | WAT | 219 | 10.817 | 26.371 | 38.798 | 1.00 | 26.12 |
| ATOM | 3472 | OH2 | WAT | 220 | 11.017 | 28.411 | 36.958 | 1.00 | 12.69 |
| ATOM | 3475 | OH2 | WAT | 221 | 8.705 | 29.831 | 37.451 | 1.00 | 23.46 |
| ATOM | 3478 | OH2 | WAT | 222 | 9.900 | 31.945 | 38.743 | 1.00 | 33.81 |
| ATOM | 3481 | OH2 | WAT | 223 | 13.883 | 32.444 | 44.288 | 1.00 | 40.76 |
| ATOM | 3484 | OH2 | WAT | 224 | 15.429 | 34.735 | 45.252 | 1.00 | 40.47 |
| ATOM | 3487 | OH2 | WAT | 225 | 21.224 | 38.749 | 36.092 | 1.00 | 33.98 |
| ATOM | 3490 | OH2 | WAT | 226 | 23.748 | 37.659 | 35.671 | 1.00 | 41.83 |
| ATOM | 3493 | OH2 | WAT | 227 | 24.333 | 39.452 | 47.629 | 1.00 | 59.51 |
| ATOM | 3496 | OH2 | WAT | 228 | 25.490 | 16.140 | 37.703 | 1.00 | 10.90 |
| ATOM | 3499 | OH2 | WAT | 229 | 26.549 | 14.796 | 35.710 | 1.00 | 49.49 |
| ATOM | 3502 | OH2 | WAT | 230 | 20.276 | 17.895 | 32.704 | 1.00 | 29.37 |
| ATOM | 3505 | OH2 | WAT | 231 | 24.010 | 15.412 | 32.805 | 1.00 | 38.09 |
| ATOM | 3508 | OH2 | WAT | 232 | 26.924 | 18.321 | 33.816 | 1.00 | 48.60 |
| ATOM | 3511 | OH2 | WAT | 233 | 29.291 | 17.381 | 32.846 | 1.00 | 33.59 |
| ATOM | 3514 | OH2 | WAT | 234 | 23.956 | 16.887 | 29.501 | 1.00 | 35.34 |
| ATOM | 3517 | OH2 | WAT | 235 | 25.804 | 14.472 | 29.865 | 1.00 | 48.14 |
| ATOM | 3520 | OH2 | WAT | 236 | 24.351 | 12.836 | 27.896 | 1.00 | 46.78 |
| ATOM | 3523 | OH2 | WAT | 237 | 25.578 | 10.706 | 26.607 | 1.00 | 52.41 |
| ATOM | 3526 | OH2 | WAT | 238 | 22.822 | 10.097 | 28.216 | 1.00 | 49.43 |
| ATOM | 3529 | OH2 | WAT | 239 | 27.474 | 16.168 | 28.090 | 1.00 | 33.81 |
| ATOM | 3532 | OH2 | WAT | 240 | 29.944 | 17.516 | 29.574 | 1.00 | 37.49 |
| ATOM | 3535 | OH2 | WAT | 241 | 30.827 | 19.605 | 27.933 | 1.00 | 32.55 |
| ATOM | 3538 | OH2 | WAT | 242 | 31.958 | 17.619 | 25.853 | 1.00 | 25.83 |
| ATOM | 3541 | OH2 | WAT | 243 | 40.784 | 11.161 | 37.660 | 1.00 | 29.72 |
| ATOM | 3544 | OH2 | WAT | 244 | 45.930 | 16.850 | 31.352 | 1.00 | 42.66 |
| ATOM | 3547 | OH2 | WAT | 245 | 20.056 | 3.884 | 22.189 | 1.00 | 49.65 |
| ATOM | 3550 | OH2 | WAT | 246 | 32.015 | 21.888 | 20.533 | 1.00 | 36.62 |
| ATOM | 3553 | OH2 | WAT | 247 | 33.308 | 19.476 | 19.839 | 1.00 | 47.65 |
| ATOM | 3556 | OH2 | WAT | 248 | 33.236 | 34.782 | 27.926 | 1.00 | 34.42 |
| ATOM | 3559 | OH2 | WAT | 249 | 37.129 | 32.062 | 32.167 | 1.00 | 43.94 |
| ATOM | 3562 | OH2 | WAT | 250 | 41.065 | 19.435 | 46.912 | 1.00 | 50.08 |
| ATOM | 3565 | OH2 | WAT | 251 | 42.232 | 17.933 | 49.143 | 1.00 | 50.59 |
| ATOM | 3568 | OH2 | WAT | 252 | 26.618 | 15.219 | 49.281 | 1.00 | 44.02 |
| ATOM | 3571 | OH2 | WAT | 253 | 30.637 | 29.550 | 11.171 | 1.00 | 12.73 |
| ATOM | 3574 | OH2 | WAT | 254 | 18.254 | 20.907 | 51.627 | 1.00 | 27.92 |
| ATOM | 3577 | OH2 | WAT | 255 | 9.554 | 27.406 | 41.176 | 1.00 | 35.17 |
| ATOM | 3580 | OH2 | WAT | 256 | 10.116 | 30.813 | 41.301 | 1.00 | 33.99 |
| ATOM | 3583 | OH2 | WAT | 257 | 0.327 | 30.797 | 32.519 | 1.00 | 32.89 |
| ATOM | 3586 | OH2 | WAT | 258 | −1.664 | 31.695 | 33.967 | 1.00 | 39.29 |
| ATOM | 3589 | OH2 | WAT | 259 | −2.387 | 39.151 | 33.967 | 1.00 | 31.35 |
| ATOM | 3592 | OH2 | WAT | 260 | −1.363 | 21.384 | 21.678 | 1.00 | 28.57 |
| ATOM | 3595 | OH2 | WAT | 261 | 4.238 | 17.555 | 13.578 | 1.00 | 38.32 |
| ATOM | 3598 | OH2 | WAT | 262 | 11.553 | 17.529 | 6.969 | 1.00 | 33.57 |
| ATOM | 3601 | OH2 | WAT | 263 | 17.099 | 12.298 | 9.712 | 1.00 | 36.46 |
| ATOM | 3604 | OH2 | WAT | 264 | 41.674 | 26.840 | 37.188 | 1.00 | 28.68 |
| ATOM | 3607 | OH2 | WAT | 265 | 40.334 | 28.815 | 35.964 | 1.00 | 26.36 |
| ATOM | 3610 | OH2 | WAT | 266 | 43.133 | 15.151 | 37.148 | 1.00 | 39.90 |
| ATOM | 3613 | OH2 | WAT | 267 | 23.457 | 27.633 | 6.838 | 1.00 | 35.51 |
| ATOM | 3616 | OH2 | WAT | 268 | 21.005 | 30.748 | 9.831 | 1.00 | 32.41 |
| ATOM | 3619 | OH2 | WAT | 269 | 18.728 | 30.160 | 10.740 | 1.00 | 33.43 |
| ATOM | 3622 | OH2 | WAT | 270 | 8.844 | 28.387 | 7.562 | 1.00 | 30.86 |
| ATOM | 3625 | OH2 | WAT | 271 | 25.931 | 18.874 | 30.011 | 1.00 | 38.08 |
| ATOM | 3628 | OH2 | WAT | 272 | 32.095 | 36.203 | 31.710 | 1.00 | 43.93 |

TABLE 3
Atomic Coordinates for
VEGFR2KD: 2,4-Dimethyl-thiazole-5-carboxylic acid {3-[4′-amino-2′-(3,4,5-trimethoxy
phenylamino)-[2,5′]bithiazolyl-4-yl]-phenyl}-amide (Compound 3)
Complex Crystalline Structure

| | | | | | | | | | |
|---|---|---|---|---|---|---|---|---|---|
| ATOM | 1 | CB | GLU | 815 | 33.991 | 16.609 | 27.663 | 1.00 | 62.40 |
| ATOM | 2 | CG | GLU | 815 | 32.918 | 17.496 | 26.725 | 1.00 | 61.57 |
| ATOM | 3 | CD | GLU | 815 | 31.200 | 17.387 | 26.650 | 1.00 | 62.06 |
| ATOM | 4 | OE1 | GLU | 815 | 30.746 | 18.118 | 27.512 | 1.00 | 58.75 |
| ATOM | 5 | OE2 | GLU | 815 | 30.352 | 16.971 | 25.758 | 1.00 | 61.31 |
| ATOM | 6 | C | GLU | 815 | 35.846 | 14.533 | 28.063 | 1.00 | 62.13 |
| ATOM | 7 | O | GLU | 815 | 36.360 | 15.399 | 28.763 | 1.00 | 63.18 |
| ATOM | 8 | HT1 | GLU | 815 | 33.556 | 14.316 | 29.229 | 1.00 | 25.00 |
| ATOM | 9 | HT2 | GLU | 815 | 33.331 | 13.255 | 27.881 | 1.00 | 25.00 |
| ATOM | 10 | N | GLU | 815 | 33.396 | 14.245 | 28.197 | 1.00 | 63.02 |
| ATOM | 11 | HT3 | GLU | 815 | 32.475 | 14.700 | 28.007 | 1.00 | 25.00 |
| ATOM | 12 | CA | GLU | 815 | 34.453 | 15.000 | 27.454 | 1.00 | 62.64 |
| ATOM | 13 | N | HIS | 816 | 36.495 | 13.298 | 27.899 | 1.00 | 61.58 |
| ATOM | 14 | H | HIS | 816 | 36.092 | 12.624 | 27.312 | 1.00 | 25.00 |
| ATOM | 15 | CA | HIS | 816 | 37.833 | 12.835 | 28.641 | 1.00 | 61.49 |
| ATOM | 16 | C | HIS | 816 | 38.444 | 13.847 | 29.737 | 1.00 | 60.13 |
| ATOM | 17 | O | HIS | 816 | 39.630 | 14.161 | 29.912 | 1.00 | 57.99 |
| ATOM | 18 | CB | HIS | 816 | 39.163 | 12.410 | 27.741 | 1.00 | 62.22 |
| ATOM | 19 | CG | HIS | 816 | 39.331 | 10.989 | 27.169 | 1.00 | 64.71 |
| ATOM | 20 | ND1 | HIS | 816 | 40.544 | 10.400 | 26.746 | 1.00 | 64.36 |
| ATOM | 21 | CE1 | HIS | 816 | 40.266 | 9.192 | 26.083 | 1.00 | 62.25 |
| ATOM | 22 | CD2 | HIS | 816 | 38.317 | 10.082 | 26.736 | 1.00 | 63.35 |
| ATOM | 23 | NE2 | HIS | 816 | 38.891 | 9.004 | 26.081 | 1.00 | 61.71 |
| ATOM | 24 | HE2 | HIS | 816 | 38.430 | 8.289 | 25.595 | 1.00 | 25.00 |
| ATOM | 25 | N | CYS | 817 | 37.451 | 14.158 | 30.625 | 1.00 | 59.44 |
| ATOM | 26 | H | CYS | 817 | 36.618 | 13.724 | 30.391 | 1.00 | 25.00 |
| ATOM | 27 | CA | CYS | 817 | 37.437 | 15.026 | 31.811 | 1.00 | 57.56 |
| ATOM | 28 | CB | CYS | 817 | 36.149 | 15.275 | 32.567 | 1.00 | 56.75 |
| ATOM | 29 | SG | CYS | 817 | 34.838 | 14.559 | 31.739 | 1.00 | 60.46 |
| ATOM | 30 | C | CYS | 817 | 38.218 | 14.505 | 32.911 | 1.00 | 56.92 |
| ATOM | 31 | O | CYS | 817 | 38.692 | 15.345 | 33.659 | 1.00 | 59.91 |
| ATOM | 32 | N | GLU | 818 | 38.307 | 13.190 | 33.124 | 1.00 | 54.69 |
| ATOM | 33 | H | GLU | 818 | 37.792 | 12.600 | 32.539 | 1.00 | 25.00 |
| ATOM | 34 | CA | GLU | 818 | 39.131 | 12.682 | 34.261 | 1.00 | 50.49 |
| ATOM | 35 | CB | GLU | 818 | 39.209 | 11.163 | 34.295 | 1.00 | 49.99 |
| ATOM | 36 | CG | GLU | 818 | 37.896 | 10.403 | 34.069 | 1.00 | 47.59 |
| ATOM | 37 | CD | GLU | 818 | 37.671 | 10.322 | 32.570 | 1.00 | 48.88 |
| ATOM | 38 | OE1 | GLU | 818 | 36.495 | 10.237 | 32.269 | 1.00 | 50.22 |
| ATOM | 39 | OE2 | GLU | 818 | 38.599 | 10.327 | 31.732 | 1.00 | 47.75 |
| ATOM | 40 | C | GLU | 818 | 40.564 | 13.231 | 34.306 | 1.00 | 49.35 |
| ATOM | 41 | O | GLU | 818 | 41.118 | 13.156 | 35.392 | 1.00 | 49.37 |
| ATOM | 42 | N | ARG | 819 | 41.173 | 13.774 | 33.208 | 1.00 | 45.97 |
| ATOM | 43 | H | ARG | 819 | 40.685 | 13.748 | 32.348 | 1.00 | 25.00 |
| ATOM | 44 | CA | ARG | 819 | 42.506 | 14.407 | 33.310 | 1.00 | 41.49 |
| ATOM | 45 | CB | ARG | 819 | 43.417 | 14.241 | 32.088 | 1.00 | 39.40 |
| ATOM | 46 | C | ARG | 819 | 42.323 | 15.933 | 33.385 | 1.00 | 41.37 |
| ATOM | 47 | O | ARG | 819 | 43.274 | 16.661 | 33.258 | 1.00 | 40.56 |
| ATOM | 48 | N | LEU | 820 | 41.113 | 16.510 | 33.415 | 1.00 | 41.55 |
| ATOM | 49 | H | LEU | 820 | 40.386 | 15.926 | 33.400 | 1.00 | 25.00 |
| ATOM | 50 | CA | LEU | 820 | 40.900 | 17.981 | 33.525 | 1.00 | 42.43 |
| ATOM | 51 | CB | LEU | 820 | 39.397 | 18.253 | 33.280 | 1.00 | 40.63 |
| ATOM | 52 | CG | LEU | 820 | 38.820 | 18.643 | 31.856 | 1.00 | 40.31 |
| ATOM | 53 | CD1 | LEU | 820 | 39.014 | 17.613 | 30.751 | 1.00 | 41.40 |
| ATOM | 54 | CD2 | LEU | 820 | 37.329 | 18.534 | 31.876 | 1.00 | 34.06 |
| ATOM | 55 | C | LEU | 820 | 41.324 | 18.413 | 34.968 | 1.00 | 44.32 |
| ATOM | 56 | O | LEU | 820 | 41.210 | 17.584 | 35.873 | 1.00 | 45.34 |
| ATOM | 57 | N | PRO | 821 | 41.870 | 19.604 | 35.339 | 1.00 | 44.72 |
| ATOM | 58 | CD | PRO | 821 | 42.686 | 20.513 | 34.543 | 1.00 | 44.44 |
| ATOM | 59 | CA | PRO | 821 | 42.085 | 19.977 | 36.770 | 1.00 | 43.39 |
| ATOM | 60 | CB | PRO | 821 | 43.379 | 20.842 | 36.791 | 1.00 | 44.61 |
| ATOM | 61 | CG | PRO | 821 | 43.982 | 20.592 | 35.408 | 1.00 | 44.10 |
| ATOM | 62 | C | PRO | 821 | 41.021 | 20.640 | 37.674 | 1.00 | 41.38 |
| ATOM | 63 | O | PRO | 821 | 39.891 | 21.095 | 37.372 | 1.00 | 38.84 |
| ATOM | 64 | N | TYR | 822 | 41.508 | 20.634 | 38.917 | 1.00 | 38.51 |
| ATOM | 65 | H | TYR | 822 | 42.406 | 20.272 | 39.073 | 1.00 | 25.00 |
| ATOM | 66 | CA | TYR | 822 | 40.715 | 21.139 | 40.001 | 1.00 | 36.09 |
| ATOM | 67 | CB | TYR | 822 | 39.989 | 20.064 | 40.741 | 1.00 | 30.28 |
| ATOM | 68 | CG | TYR | 822 | 39.070 | 20.793 | 41.712 | 1.00 | 27.05 |
| ATOM | 69 | CD1 | TYR | 822 | 39.305 | 20.602 | 43.040 | 1.00 | 23.15 |
| ATOM | 70 | CE1 | TYR | 822 | 38.530 | 21.226 | 43.986 | 1.00 | 18.26 |
| ATOM | 71 | CD2 | TYR | 822 | 37.986 | 21.680 | 41.367 | 1.00 | 25.83 |
| ATOM | 72 | CE2 | TYR | 822 | 37.203 | 22.318 | 42.312 | 1.00 | 19.73 |
| ATOM | 73 | CZ | TYR | 822 | 37.534 | 22.054 | 43.626 | 1.00 | 18.93 |
| ATOM | 74 | OH | TYR | 822 | 36.852 | 22.553 | 44.703 | 1.00 | 21.59 |
| ATOM | 75 | HH | TYR | 822 | 36.139 | 23.117 | 44.412 | 1.00 | 25.00 |
| ATOM | 76 | C | TYR | 822 | 41.490 | 21.874 | 41.051 | 1.00 | 36.67 |
| ATOM | 77 | O | TYR | 822 | 42.053 | 21.270 | 41.972 | 1.00 | 36.77 |
| ATOM | 78 | N | ASP | 823 | 41.358 | 23.219 | 40.898 | 1.00 | 36.35 |
| ATOM | 79 | H | ASP | 823 | 40.764 | 23.503 | 40.184 | 1.00 | 25.00 |
| ATOM | 80 | CA | ASP | 823 | 41.918 | 24.287 | 41.674 | 1.00 | 32.06 |
| ATOM | 81 | CB | ASP | 823 | 41.837 | 25.547 | 40.805 | 1.00 | 34.96 |
| ATOM | 82 | CG | ASP | 823 | 43.062 | 26.404 | 41.103 | 1.00 | 38.03 |
| ATOM | 83 | OD1 | ASP | 823 | 43.505 | 26.361 | 42.253 | 1.00 | 42.10 |
| ATOM | 84 | OD2 | ASP | 823 | 43.589 | 27.040 | 40.168 | 1.00 | 38.16 |
| ATOM | 85 | C | ASP | 823 | 41.243 | 24.509 | 42.946 | 1.00 | 30.61 |
| ATOM | 86 | O | ASP | 823 | 40.474 | 25.421 | 43.007 | 1.00 | 32.34 |
| ATOM | 87 | N | ALA | 824 | 41.423 | 23.817 | 44.012 | 1.00 | 31.62 |
| ATOM | 88 | H | ALA | 824 | 42.098 | 23.098 | 44.046 | 1.00 | 25.00 |
| ATOM | 89 | CA | ALA | 824 | 40.653 | 24.161 | 45.247 | 1.00 | 32.98 |
| ATOM | 90 | CB | ALA | 824 | 41.072 | 23.218 | 46.431 | 1.00 | 34.30 |
| ATOM | 91 | C | ALA | 824 | 40.838 | 25.617 | 45.726 | 1.00 | 33.91 |
| ATOM | 92 | O | ALA | 824 | 39.947 | 26.181 | 46.373 | 1.00 | 34.58 |
| ATOM | 93 | N | SER | 825 | 42.025 | 26.197 | 45.506 | 1.00 | 32.18 |
| ATOM | 94 | H | SER | 825 | 42.738 | 25.696 | 45.067 | 1.00 | 25.00 |
| ATOM | 95 | CA | SER | 825 | 42.224 | 27.583 | 45.929 | 1.00 | 32.44 |
| ATOM | 96 | CB | SER | 825 | 43.572 | 28.125 | 45.583 | 1.00 | 31.80 |
| ATOM | 97 | OG | SER | 825 | 43.804 | 27.616 | 44.343 | 1.00 | 29.72 |
| ATOM | 98 | HG | SER | 825 | 44.623 | 27.963 | 43.983 | 1.00 | 25.00 |
| ATOM | 99 | C | SER | 825 | 41.224 | 28.477 | 45.291 | 1.00 | 33.75 |
| ATOM | 100 | O | SER | 825 | 40.634 | 29.400 | 45.893 | 1.00 | 35.61 |
| ATOM | 101 | N | LYS | 826 | 41.045 | 28.154 | 44.019 | 1.00 | 31.92 |
| ATOM | 102 | H | LYS | 826 | 41.581 | 27.482 | 43.566 | 1.00 | 25.00 |
| ATOM | 103 | CA | LYS | 826 | 39.993 | 28.952 | 43.396 | 1.00 | 29.44 |
| ATOM | 104 | CB | LYS | 826 | 40.110 | 29.054 | 41.889 | 1.00 | 26.38 |
| ATOM | 105 | CG | LYS | 826 | 38.901 | 29.979 | 41.482 | 1.00 | 24.13 |
| ATOM | 106 | CD | LYS | 826 | 39.302 | 30.257 | 39.995 | 1.00 | 23.39 |
| ATOM | 107 | CE | LYS | 826 | 38.310 | 30.944 | 39.071 | 1.00 | 19.13 |
| ATOM | 108 | NZ | LYS | 826 | 39.087 | 30.967 | 37.801 | 1.00 | 22.11 |
| ATOM | 109 | HZ1 | LYS | 826 | 39.597 | 30.084 | 37.610 | 1.00 | 25.00 |
| ATOM | 110 | HZ2 | LYS | 826 | 39.780 | 31.739 | 37.855 | 1.00 | 25.00 |
| ATOM | 111 | HZ3 | LYS | 826 | 38.469 | 31.182 | 36.998 | 1.00 | 25.00 |
| ATOM | 112 | C | LYS | 826 | 38.658 | 28.413 | 43.718 | 1.00 | 27.43 |
| ATOM | 113 | O | LYS | 826 | 37.972 | 29.049 | 44.473 | 1.00 | 31.68 |
| ATOM | 114 | N | TRP | 827 | 38.211 | 27.223 | 43.444 | 1.00 | 24.25 |
| ATOM | 115 | H | TRP | 827 | 38.823 | 26.546 | 43.106 | 1.00 | 25.00 |
| ATOM | 116 | CA | TRP | 827 | 36.790 | 26.927 | 43.772 | 1.00 | 19.81 |
| ATOM | 117 | CB | TRP | 827 | 36.301 | 25.942 | 42.781 | 1.00 | 15.43 |
| ATOM | 118 | CG | TRP | 827 | 36.722 | 26.467 | 41.484 | 1.00 | 8.12 |
| ATOM | 119 | CD2 | TRP | 827 | 36.040 | 27.365 | 40.657 | 1.00 | 4.68 |
| ATOM | 120 | CE2 | TRP | 827 | 36.767 | 27.536 | 39.525 | 1.00 | 6.68 |
| ATOM | 121 | CE3 | TRP | 827 | 34.855 | 28.030 | 40.812 | 1.00 | 10.17 |
| ATOM | 122 | CD1 | TRP | 827 | 37.823 | 26.151 | 40.797 | 1.00 | 5.86 |
| ATOM | 123 | NE1 | TRP | 827 | 37.861 | 26.777 | 39.570 | 1.00 | 10.84 |
| ATOM | 124 | HE1 | TRP | 827 | 38.626 | 26.865 | 38.956 | 1.00 | 25.00 |
| ATOM | 125 | CZ2 | TRP | 827 | 36.233 | 28.323 | 38.540 | 1.00 | 11.79 |
| ATOM | 126 | CZ3 | TRP | 827 | 34.288 | 28.855 | 39.866 | 1.00 | 13.12 |
| ATOM | 127 | CH2 | TRP | 827 | 35.028 | 29.020 | 38.725 | 1.00 | 11.92 |
| ATOM | 128 | C | TRP | 827 | 36.306 | 26.436 | 45.069 | 1.00 | 21.07 |
| ATOM | 129 | O | TRP | 827 | 35.097 | 26.308 | 45.301 | 1.00 | 21.47 |
| ATOM | 130 | N | GLU | 828 | 37.137 | 26.057 | 45.963 | 1.00 | 21.26 |
| ATOM | 131 | H | GLU | 828 | 38.075 | 26.310 | 45.968 | 1.00 | 25.00 |
| ATOM | 132 | CA | GLU | 828 | 36.474 | 25.368 | 47.072 | 1.00 | 21.86 |
| ATOM | 133 | CB | GLU | 828 | 37.639 | 24.613 | 47.756 | 1.00 | 23.30 |
| ATOM | 134 | CG | GLU | 828 | 37.345 | 23.560 | 48.888 | 1.00 | 19.85 |
| ATOM | 135 | CD | GLU | 828 | 36.525 | 22.381 | 48.429 | 1.00 | 21.41 |
| ATOM | 136 | OE1 | GLU | 828 | 35.646 | 21.922 | 49.157 | 1.00 | 18.71 |
| ATOM | 137 | OE2 | GLU | 828 | 36.779 | 21.978 | 47.297 | 1.00 | 23.69 |
| ATOM | 138 | C | GLU | 828 | 35.658 | 26.180 | 47.969 | 1.00 | 22.93 |
| ATOM | 139 | O | GLU | 828 | 36.083 | 27.252 | 48.290 | 1.00 | 25.82 |
| ATOM | 140 | N | PHE | 829 | 34.593 | 25.752 | 48.551 | 1.00 | 23.26 |
| ATOM | 141 | H | PHE | 829 | 34.253 | 24.868 | 48.312 | 1.00 | 25.00 |
| ATOM | 142 | CA | PHE | 829 | 33.916 | 26.635 | 49.418 | 1.00 | 24.85 |
| ATOM | 143 | CB | PHE | 829 | 32.690 | 27.259 | 48.638 | 1.00 | 23.46 |
| ATOM | 144 | CG | PHE | 829 | 31.877 | 28.154 | 49.557 | 1.00 | 23.16 |
| ATOM | 145 | CD1 | PHE | 829 | 32.265 | 29.471 | 49.862 | 1.00 | 20.57 |
| ATOM | 146 | CD2 | PHE | 829 | 30.673 | 27.712 | 50.142 | 1.00 | 24.45 |
| ATOM | 147 | CE1 | PHE | 829 | 31.442 | 30.249 | 50.709 | 1.00 | 20.21 |
| ATOM | 148 | CE2 | PHE | 829 | 29.816 | 28.460 | 50.999 | 1.00 | 19.49 |
| ATOM | 149 | CZ | PHE | 829 | 30.241 | 29.756 | 51.265 | 1.00 | 21.45 |
| ATOM | 150 | C | PHE | 829 | 33.520 | 25.875 | 50.669 | 1.00 | 28.35 |
| ATOM | 151 | O | PHE | 829 | 32.975 | 24.774 | 50.699 | 1.00 | 31.85 |
| ATOM | 152 | N | PRO | 830 | 33.783 | 26.439 | 51.812 | 1.00 | 30.78 |
| ATOM | 153 | CD | PRO | 830 | 34.647 | 27.626 | 51.940 | 1.00 | 30.70 |
| ATOM | 154 | CA | PRO | 830 | 33.480 | 25.854 | 53.090 | 1.00 | 28.75 |
| ATOM | 155 | CB | PRO | 830 | 33.803 | 26.897 | 54.062 | 1.00 | 29.72 |
| ATOM | 156 | CG | PRO | 830 | 34.970 | 27.594 | 53.373 | 1.00 | 28.14 |
| ATOM | 157 | C | PRO | 830 | 32.144 | 25.380 | 53.300 | 1.00 | 30.22 |
| ATOM | 158 | O | PRO | 830 | 31.248 | 26.159 | 53.571 | 1.00 | 31.28 |
| ATOM | 159 | N | ARG | 831 | 31.978 | 24.125 | 53.472 | 1.00 | 30.84 |
| ATOM | 160 | H | ARG | 831 | 32.708 | 23.484 | 53.476 | 1.00 | 25.00 |
| ATOM | 161 | CA | ARG | 831 | 30.583 | 23.694 | 53.718 | 1.00 | 32.53 |
| ATOM | 162 | CB | ARG | 831 | 30.658 | 22.057 | 53.725 | 1.00 | 36.60 |
| ATOM | 163 | CG | ARG | 831 | 29.577 | 20.867 | 53.694 | 1.00 | 35.93 |
| ATOM | 164 | CD | ARG | 831 | 29.889 | 19.666 | 52.626 | 1.00 | 35.41 |
| ATOM | 165 | NE | ARG | 831 | 31.195 | 19.024 | 52.784 | 1.00 | 32.49 |
| ATOM | 166 | HE | ARG | 831 | 31.616 | 19.009 | 53.662 | 1.00 | 25.00 |
| ATOM | 167 | CZ | ARG | 831 | 31.798 | 18.365 | 51.828 | 1.00 | 30.04 |
| ATOM | 168 | NH1 | ARG | 831 | 32.957 | 17.856 | 52.059 | 1.00 | 27.73 |
| ATOM | 169 | HH11 | ARG | 831 | 33.373 | 17.940 | 52.961 | 1.00 | 25.00 |
| ATOM | 170 | HH12 | ARG | 831 | 33.427 | 17.392 | 51.311 | 1.00 | 25.00 |
| ATOM | 171 | NH2 | ARG | 831 | 31.387 | 18.294 | 50.601 | 1.00 | 27.45 |
| ATOM | 172 | HH21 | ARG | 831 | 30.536 | 18.736 | 50.322 | 1.00 | 25.00 |
| ATOM | 173 | HH22 | ARG | 831 | 31.937 | 17.791 | 49.929 | 1.00 | 25.00 |
| ATOM | 174 | C | ARG | 831 | 30.045 | 24.386 | 54.994 | 1.00 | 32.07 |
| ATOM | 175 | O | ARG | 831 | 28.853 | 24.549 | 55.202 | 1.00 | 33.33 |
| ATOM | 176 | N | ASP | 832 | 30.923 | 24.880 | 55.880 | 1.00 | 34.96 |
| ATOM | 177 | H | ASP | 832 | 31.870 | 24.841 | 55.637 | 1.00 | 25.00 |
| ATOM | 178 | CA | ASP | 832 | 30.455 | 25.551 | 57.151 | 1.00 | 37.09 |
| ATOM | 179 | CB | ASP | 832 | 31.670 | 25.876 | 58.215 | 1.00 | 39.85 |
| ATOM | 180 | CG | ASP | 832 | 33.039 | 26.526 | 57.826 | 1.00 | 44.17 |
| ATOM | 181 | OD1 | ASP | 832 | 33.745 | 25.944 | 57.019 | 1.00 | 47.79 |
| ATOM | 182 | OD2 | ASP | 832 | 33.484 | 27.565 | 58.363 | 1.00 | 46.89 |
| ATOM | 183 | C | ASP | 832 | 29.687 | 26.845 | 56.857 | 1.00 | 35.94 |
| ATOM | 184 | O | ASP | 832 | 28.762 | 27.147 | 57.628 | 1.00 | 33.47 |
| ATOM | 185 | N | ARG | 833 | 30.167 | 27.545 | 55.770 | 1.00 | 33.36 |
| ATOM | 186 | H | ARG | 833 | 30.896 | 27.130 | 55.268 | 1.00 | 25.00 |
| ATOM | 187 | CA | ARG | 833 | 29.697 | 28.800 | 55.204 | 1.00 | 32.72 |
| ATOM | 188 | CB | ARG | 833 | 30.858 | 29.505 | 54.474 | 1.00 | 26.77 |
| ATOM | 189 | CG | ARG | 833 | 32.079 | 29.630 | 55.361 | 1.00 | 28.24 |
| ATOM | 190 | CD | ARG | 833 | 32.584 | 31.038 | 55.552 | 1.00 | 33.81 |
| ATOM | 191 | NE | ARG | 833 | 33.862 | 31.187 | 54.873 | 1.00 | 39.69 |
| ATOM | 192 | HE | ARG | 833 | 33.852 | 31.464 | 53.937 | 1.00 | 25.00 |
| ATOM | 193 | CZ | ARG | 833 | 35.044 | 30.789 | 55.436 | 1.00 | 43.00 |
| ATOM | 194 | NH1 | ARG | 833 | 36.213 | 30.938 | 54.746 | 1.00 | 42.18 |
| ATOM | 195 | HH11 | ARG | 833 | 36.199 | 31.316 | 53.811 | 1.00 | 25.00 |
| ATOM | 196 | HH12 | ARG | 833 | 37.095 | 30.697 | 55.151 | 1.00 | 25.00 |
| ATOM | 197 | NH2 | ARG | 833 | 35.156 | 30.262 | 56.674 | 1.00 | 41.37 |
| ATOM | 198 | HH21 | ARG | 833 | 34.366 | 30.097 | 57.255 | 1.00 | 25.00 |
| ATOM | 199 | HH22 | ARG | 833 | 36.087 | 30.057 | 56.990 | 1.00 | 25.00 |
| ATOM | 200 | C | ARG | 833 | 28.539 | 28.539 | 54.207 | 1.00 | 37.23 |
| ATOM | 201 | O | ARG | 833 | 28.259 | 29.365 | 53.334 | 1.00 | 42.04 |
| ATOM | 202 | N | LEU | 834 | 27.668 | 27.543 | 54.324 | 1.00 | 37.24 |
| ATOM | 203 | H | LEU | 834 | 27.755 | 26.931 | 55.095 | 1.00 | 25.00 |
| ATOM | 204 | CA | LEU | 834 | 26.641 | 27.334 | 53.262 | 1.00 | 35.52 |
| ATOM | 205 | CB | LEU | 834 | 27.209 | 26.351 | 52.217 | 1.00 | 33.79 |
| ATOM | 206 | CG | LEU | 834 | 26.375 | 25.903 | 51.016 | 1.00 | 36.17 |
| ATOM | 207 | CD1 | LEU | 834 | 26.337 | 27.107 | 49.992 | 1.00 | 36.58 |
| ATOM | 208 | CD2 | LEU | 834 | 27.046 | 24.759 | 50.225 | 1.00 | 35.51 |
| ATOM | 209 | C | LEU | 834 | 25.567 | 26.715 | 54.013 | 1.00 | 34.92 |
| ATOM | 210 | O | LEU | 834 | 25.868 | 25.681 | 54.595 | 1.00 | 38.86 |
| ATOM | 211 | N | LYS | 835 | 24.463 | 27.391 | 54.152 | 1.00 | 32.53 |
| ATOM | 212 | H | LYS | 835 | 24.420 | 28.265 | 53.700 | 1.00 | 25.00 |
| ATOM | 213 | CA | LYS | 835 | 23.325 | 26.873 | 54.853 | 1.00 | 33.81 |
| ATOM | 214 | CB | LYS | 835 | 22.666 | 27.942 | 55.742 | 1.00 | 32.61 |
| ATOM | 215 | C | LYS | 835 | 22.248 | 26.344 | 53.865 | 1.00 | 35.25 |
| ATOM | 216 | O | LYS | 835 | 21.419 | 27.064 | 53.326 | 1.00 | 37.84 |
| ATOM | 217 | N | LEU | 836 | 22.240 | 25.070 | 53.517 | 1.00 | 35.72 |
| ATOM | 218 | H | LEU | 836 | 22.868 | 24.492 | 54.003 | 1.00 | 25.00 |
| ATOM | 219 | CA | LEU | 836 | 21.266 | 24.426 | 52.570 | 1.00 | 35.00 |
| ATOM | 220 | CB | LEU | 836 | 21.570 | 22.914 | 52.569 | 1.00 | 36.82 |
| ATOM | 221 | CG | LEU | 836 | 22.677 | 22.463 | 51.630 | 1.00 | 35.64 |
| ATOM | 222 | CD1 | LEU | 836 | 23.897 | 23.377 | 51.794 | 1.00 | 34.58 |
| ATOM | 223 | CD2 | LEU | 836 | 22.940 | 20.955 | 51.892 | 1.00 | 35.53 |
| ATOM | 224 | C | LEU | 836 | 19.823 | 24.666 | 52.972 | 1.00 | 33.72 |
| ATOM | 225 | O | LEU | 836 | 19.616 | 24.865 | 54.129 | 1.00 | 35.10 |
| ATOM | 226 | N | GLY | 837 | 18.830 | 24.590 | 52.138 | 1.00 | 32.77 |
| ATOM | 227 | H | GLY | 837 | 19.030 | 24.205 | 51.279 | 1.00 | 25.00 |
| ATOM | 228 | CA | GLY | 837 | 17.439 | 24.822 | 52.449 | 1.00 | 34.34 |
| ATOM | 229 | C | GLY | 837 | 16.574 | 23.893 | 51.560 | 1.00 | 39.07 |
| ATOM | 230 | O | GLY | 837 | 16.804 | 22.678 | 51.380 | 1.00 | 41.75 |
| ATOM | 231 | N | LYS | 838 | 15.553 | 24.531 | 50.999 | 1.00 | 39.26 |
| ATOM | 232 | H | LYS | 838 | 15.403 | 25.409 | 51.405 | 1.00 | 25.00 |
| ATOM | 233 | CA | LYS | 838 | 14.536 | 23.925 | 50.113 | 1.00 | 38.74 |
| ATOM | 234 | CB | LYS | 838 | 13.706 | 24.963 | 49.451 | 1.00 | 44.66 |
| ATOM | 235 | CG | LYS | 838 | 12.279 | 24.684 | 49.759 | 1.00 | 54.74 |
| ATOM | 236 | CD | LYS | 838 | 11.395 | 25.284 | 48.580 | 1.00 | 61.61 |
| ATOM | 237 | CE | LYS | 838 | 9.843 | 24.934 | 48.756 | 1.00 | 64.92 |
| ATOM | 238 | NZ | LYS | 838 | 9.359 | 25.203 | 50.162 | 1.00 | 66.58 |
| ATOM | 239 | HZ1 | LYS | 838 | 9.732 | 26.121 | 50.480 | 1.00 | 25.00 |
| ATOM | 240 | HZ2 | LYS | 838 | 9.725 | 24.460 | 50.793 | 1.00 | 25.00 |
| ATOM | 241 | HZ3 | LYS | 838 | 8.320 | 25.216 | 50.177 | 1.00 | 25.00 |
| ATOM | 242 | C | LYS | 838 | 15.161 | 23.221 | 49.002 | 1.00 | 34.77 |
| ATOM | 243 | O | LYS | 838 | 16.242 | 23.591 | 48.563 | 1.00 | 33.66 |
| ATOM | 244 | N | PRO | 839 | 14.493 | 22.291 | 48.438 | 1.00 | 33.56 |
| ATOM | 245 | CD | PRO | 839 | 13.842 | 21.263 | 49.202 | 1.00 | 35.92 |
| ATOM | 246 | CA | PRO | 839 | 14.861 | 21.776 | 47.092 | 1.00 | 33.20 |
| ATOM | 247 | CB | PRO | 839 | 14.661 | 20.228 | 47.079 | 1.00 | 32.57 |
| ATOM | 248 | CG | PRO | 839 | 13.617 | 20.106 | 48.169 | 1.00 | 34.28 |
| ATOM | 249 | C | PRO | 839 | 14.161 | 22.406 | 45.916 | 1.00 | 31.60 |
| ATOM | 250 | O | PRO | 839 | 13.009 | 22.783 | 45.886 | 1.00 | 31.46 |
| ATOM | 251 | N | LEU | 840 | 14.949 | 22.630 | 44.920 | 1.00 | 31.36 |
| ATOM | 252 | H | LEU | 840 | 15.870 | 22.314 | 44.999 | 1.00 | 25.00 |
| ATOM | 253 | CA | LEU | 840 | 14.485 | 23.190 | 43.691 | 1.00 | 29.73 |
| ATOM | 254 | CB | LEU | 840 | 15.604 | 24.190 | 43.393 | 1.00 | 27.12 |
| ATOM | 255 | CG | LEU | 840 | 15.968 | 25.242 | 44.459 | 1.00 | 22.77 |
| ATOM | 256 | CD1 | LEU | 840 | 16.838 | 26.293 | 43.841 | 1.00 | 16.75 |
| ATOM | 257 | CD2 | LEU | 840 | 14.723 | 25.958 | 44.950 | 1.00 | 23.28 |
| ATOM | 258 | C | LEU | 840 | 14.239 | 22.012 | 42.645 | 1.00 | 33.13 |
| ATOM | 259 | O | LEU | 840 | 13.297 | 21.964 | 41.847 | 1.00 | 36.34 |
| ATOM | 260 | N | GLY | 841 | 15.034 | 20.958 | 42.602 | 1.00 | 33.70 |
| ATOM | 261 | H | GLY | 841 | 15.841 | 20.923 | 43.154 | 1.00 | 25.00 |
| ATOM | 262 | CA | GLY | 841 | 14.802 | 19.902 | 41.641 | 1.00 | 34.72 |
| ATOM | 263 | C | GLY | 841 | 15.043 | 18.643 | 42.359 | 1.00 | 38.25 |
| ATOM | 264 | O | GLY | 841 | 15.948 | 18.633 | 43.181 | 1.00 | 39.10 |
| ATOM | 265 | N | ARG | 842 | 14.333 | 17.561 | 42.109 | 1.00 | 39.98 |
| ATOM | 266 | H | ARG | 842 | 13.615 | 17.548 | 41.443 | 1.00 | 25.00 |
| ATOM | 267 | CA | ARG | 842 | 14.678 | 16.382 | 42.877 | 1.00 | 41.50 |
| ATOM | 268 | CB | ARG | 842 | 13.760 | 16.376 | 44.098 | 1.00 | 45.18 |
| ATOM | 269 | CG | ARG | 842 | 14.733 | 15.755 | 45.117 | 1.00 | 49.99 |
| ATOM | 270 | CD | ARG | 842 | 14.310 | 15.988 | 46.549 | 1.00 | 56.75 |
| ATOM | 271 | NE | ARG | 842 | 15.020 | 14.978 | 47.333 | 1.00 | 66.10 |
| ATOM | 272 | HE | ARG | 842 | 15.559 | 14.319 | 46.855 | 1.00 | 25.00 |
| ATOM | 273 | CZ | ARG | 842 | 14.951 | 14.905 | 48.680 | 1.00 | 71.98 |
| ATOM | 274 | NH1 | ARG | 842 | 15.631 | 13.930 | 49.312 | 1.00 | 73.67 |
| ATOM | 275 | HH11 | ARG | 842 | 16.170 | 13.275 | 48.780 | 1.00 | 25.00 |
| ATOM | 276 | HH12 | ARG | 842 | 15.622 | 13.873 | 50.312 | 1.00 | 25.00 |
| ATOM | 277 | NH2 | ARG | 842 | 14.299 | 15.830 | 49.419 | 1.00 | 74.04 |
| ATOM | 278 | HH21 | ARG | 842 | 13.846 | 16.599 | 48.974 | 1.00 | 25.00 |
| ATOM | 279 | HH22 | ARG | 842 | 14.301 | 15.751 | 50.413 | 1.00 | 25.00 |
| ATOM | 280 | C | ARG | 842 | 14.640 | 15.061 | 42.143 | 1.00 | 39.98 |
| ATOM | 281 | O | ARG | 842 | 13.616 | 14.655 | 41.623 | 1.00 | 39.02 |
| ATOM | 282 | N | GLY | 843 | 15.758 | 14.407 | 41.980 | 1.00 | 40.68 |
| ATOM | 283 | H | GLY | 843 | 16.624 | 14.815 | 42.189 | 1.00 | 25.00 |
| ATOM | 284 | CA | GLY | 843 | 15.667 | 13.111 | 41.289 | 1.00 | 43.15 |
| ATOM | 285 | C | GLY | 843 | 15.691 | 11.943 | 42.264 | 1.00 | 41.90 |
| ATOM | 286 | O | GLY | 843 | 15.033 | 11.993 | 43.304 | 1.00 | 37.57 |
| ATOM | 287 | N | ALA | 844 | 16.292 | 10.835 | 41.839 | 1.00 | 41.30 |
| ATOM | 288 | H | ALA | 844 | 16.623 | 10.755 | 40.930 | 1.00 | 25.00 |
| ATOM | 289 | CA | ALA | 844 | 16.380 | 9.738 | 42.792 | 1.00 | 42.36 |
| ATOM | 290 | CB | ALA | 844 | 15.880 | 8.463 | 42.015 | 1.00 | 44.08 |
| ATOM | 291 | C | ALA | 844 | 17.841 | 9.660 | 43.357 | 1.00 | 42.17 |
| ATOM | 292 | O | ALA | 844 | 18.146 | 9.350 | 44.504 | 1.00 | 45.45 |
| ATOM | 293 | N | PHE | 845 | 18.857 | 9.943 | 42.590 | 1.00 | 41.90 |
| ATOM | 294 | H | PHE | 845 | 18.710 | 10.195 | 41.651 | 1.00 | 25.00 |
| ATOM | 295 | CA | PHE | 845 | 20.231 | 9.911 | 43.157 | 1.00 | 41.54 |
| ATOM | 296 | CB | PHE | 845 | 21.176 | 9.008 | 42.327 | 1.00 | 46.51 |
| ATOM | 297 | CG | PHE | 845 | 20.444 | 7.768 | 42.064 | 1.00 | 49.94 |
| ATOM | 298 | CD1 | PHE | 845 | 20.508 | 7.308 | 40.755 | 1.00 | 52.03 |
| ATOM | 299 | CD2 | PHE | 845 | 19.622 | 7.161 | 43.031 | 1.00 | 49.62 |
| ATOM | 300 | CE1 | PHE | 845 | 19.683 | 6.256 | 40.391 | 1.00 | 56.03 |
| ATOM | 301 | CE2 | PHE | 845 | 18.797 | 6.114 | 42.676 | 1.00 | 51.34 |
| ATOM | 302 | CZ | PHE | 845 | 18.845 | 5.667 | 41.358 | 1.00 | 55.11 |
| ATOM | 303 | C | PHE | 845 | 20.814 | 11.263 | 43.156 | 1.00 | 38.11 |
| ATOM | 304 | O | PHE | 845 | 22.029 | 11.399 | 43.249 | 1.00 | 38.23 |
| ATOM | 305 | N | GLY | 846 | 19.996 | 12.280 | 42.942 | 1.00 | 35.62 |
| ATOM | 306 | H | GLY | 846 | 19.038 | 12.131 | 42.769 | 1.00 | 25.00 |
| ATOM | 307 | CA | GLY | 846 | 20.546 | 13.647 | 42.910 | 1.00 | 31.14 |
| ATOM | 308 | C | GLY | 846 | 19.491 | 14.685 | 43.217 | 1.00 | 28.47 |
| ATOM | 309 | O | GLY | 846 | 18.296 | 14.354 | 43.276 | 1.00 | 26.84 |
| ATOM | 310 | N | GLN | 847 | 19.925 | 15.907 | 43.521 | 1.00 | 24.28 |
| ATOM | 311 | H | GLN | 847 | 20.883 | 16.070 | 43.619 | 1.00 | 25.00 |
| ATOM | 312 | CA | GLN | 847 | 18.963 | 16.980 | 43.735 | 1.00 | 23.43 |
| ATOM | 313 | CB | GLN | 847 | 18.288 | 16.892 | 45.121 | 1.00 | 23.43 |
| ATOM | 314 | CG | GLN | 847 | 19.313 | 17.088 | 46.128 | 1.00 | 27.59 |
| ATOM | 315 | CD | GLN | 847 | 18.832 | 16.636 | 47.437 | 1.00 | 31.89 |
| ATOM | 316 | OE1 | GLN | 847 | 17.690 | 16.858 | 47.810 | 1.00 | 35.85 |
| ATOM | 317 | NE2 | GLN | 847 | 19.725 | 15.973 | 48.174 | 1.00 | 34.52 |
| ATOM | 318 | HE21 | GLN | 847 | 20.615 | 15.820 | 47.817 | 1.00 | 25.00 |
| ATOM | 319 | HE22 | GLN | 847 | 19.514 | 15.621 | 49.060 | 1.00 | 25.00 |
| ATOM | 320 | C | GLN | 847 | 19.592 | 18.288 | 43.591 | 1.00 | 20.88 |
| ATOM | 321 | O | GLN | 847 | 20.773 | 18.343 | 43.299 | 1.00 | 20.69 |
| ATOM | 322 | N | VAL | 848 | 18.777 | 19.350 | 43.491 | 1.00 | 22.04 |
| ATOM | 323 | H | VAL | 848 | 17.796 | 19.226 | 43.447 | 1.00 | 25.00 |
| ATOM | 324 | CA | VAL | 848 | 19.318 | 20.738 | 43.427 | 1.00 | 19.20 |
| ATOM | 325 | CB | VAL | 848 | 19.182 | 21.514 | 42.137 | 1.00 | 16.52 |
| ATOM | 326 | CG1 | VAL | 848 | 19.678 | 22.991 | 42.202 | 1.00 | 12.88 |
| ATOM | 327 | CG2 | VAL | 848 | 20.216 | 20.834 | 41.246 | 1.00 | 13.88 |
| ATOM | 328 | C | VAL | 848 | 18.597 | 21.473 | 44.454 | 1.00 | 18.10 |
| ATOM | 329 | O | VAL | 848 | 17.392 | 21.403 | 44.614 | 1.00 | 15.79 |
| ATOM | 330 | N | ILE | 849 | 19.379 | 21.924 | 45.373 | 1.00 | 20.84 |
| ATOM | 331 | H | ILE | 849 | 20.354 | 21.890 | 45.335 | 1.00 | 25.00 |
| ATOM | 332 | CA | ILE | 849 | 18.646 | 22.621 | 46.425 | 1.00 | 26.63 |
| ATOM | 333 | CB | ILE | 849 | 18.738 | 21.823 | 47.792 | 1.00 | 25.44 |
| ATOM | 334 | CG2 | ILE | 849 | 18.189 | 20.394 | 47.610 | 1.00 | 29.87 |
| ATOM | 335 | CG1 | ILE | 849 | 20.113 | 21.690 | 48.292 | 1.00 | 21.18 |
| ATOM | 336 | CD1 | ILE | 849 | 19.398 | 22.131 | 49.599 | 1.00 | 26.55 |
| ATOM | 337 | C | ILE | 849 | 19.110 | 24.039 | 46.627 | 1.00 | 28.80 |
| ATOM | 338 | O | ILE | 849 | 20.175 | 24.451 | 46.118 | 1.00 | 27.88 |
| ATOM | 339 | N | GLU | 850 | 18.203 | 24.797 | 47.218 | 1.00 | 31.58 |
| ATOM | 340 | H | GLU | 850 | 17.292 | 24.443 | 47.338 | 1.00 | 25.00 |
| ATOM | 341 | CA | GLU | 850 | 18.494 | 26.202 | 47.530 | 1.00 | 33.54 |
| ATOM | 342 | CB | GLU | 850 | 17.191 | 26.964 | 47.674 | 1.00 | 36.15 |
| ATOM | 343 | CG | GLU | 850 | 17.349 | 28.483 | 47.785 | 1.00 | 39.13 |
| ATOM | 344 | CD | GLU | 850 | 15.999 | 29.174 | 47.711 | 1.00 | 41.63 |
| ATOM | 345 | OE1 | GLU | 850 | 14.924 | 28.536 | 47.704 | 1.00 | 45.62 |
| ATOM | 346 | OE2 | GLU | 850 | 16.011 | 30.393 | 47.729 | 1.00 | 42.76 |
| ATOM | 347 | C | GLU | 850 | 19.283 | 26.331 | 48.785 | 1.00 | 32.25 |
| ATOM | 348 | O | GLU | 850 | 19.074 | 25.608 | 49.741 | 1.00 | 33.72 |
| ATOM | 349 | N | ALA | 851 | 20.174 | 27.289 | 48.823 | 1.00 | 30.65 |
| ATOM | 350 | H | ALA | 851 | 20.342 | 27.842 | 48.046 | 1.00 | 25.00 |
| ATOM | 351 | CA | ALA | 851 | 21.015 | 27.522 | 49.972 | 1.00 | 26.57 |
| ATOM | 352 | CB | ALA | 851 | 22.280 | 26.663 | 49.814 | 1.00 | 22.69 |
| ATOM | 353 | C | ALA | 851 | 21.407 | 28.993 | 49.925 | 1.00 | 26.28 |
| ATOM | 354 | O | ALA | 851 | 21.364 | 29.694 | 48.925 | 1.00 | 26.60 |
| ATOM | 355 | N | ASP | 852 | 21.908 | 29.430 | 51.046 | 1.00 | 26.10 |
| ATOM | 356 | H | ASP | 852 | 21.912 | 28.776 | 51.773 | 1.00 | 25.00 |
| ATOM | 357 | CA | ASP | 852 | 22.471 | 30.759 | 51.388 | 1.00 | 24.66 |
| ATOM | 358 | CB | ASP | 852 | 21.654 | 31.275 | 52.582 | 1.00 | 24.59 |
| ATOM | 359 | CG | ASP | 852 | 20.253 | 31.620 | 52.132 | 1.00 | 22.42 |
| ATOM | 360 | OD1 | ASP | 852 | 19.380 | 31.811 | 52.944 | 1.00 | 28.50 |
| ATOM | 361 | OD2 | ASP | 852 | 19.991 | 31.716 | 50.968 | 1.00 | 25.96 |
| ATOM | 362 | C | ASP | 852 | 23.923 | 30.606 | 51.709 | 1.00 | 20.58 |
| ATOM | 363 | O | ASP | 852 | 24.170 | 29.804 | 52.563 | 1.00 | 21.49 |
| ATOM | 364 | N | ALA | 853 | 24.805 | 31.185 | 50.940 | 1.00 | 19.60 |
| ATOM | 365 | H | ALA | 853 | 24.412 | 31.674 | 50.194 | 1.00 | 25.00 |
| ATOM | 366 | CA | ALA | 853 | 26.219 | 31.172 | 51.056 | 1.00 | 23.85 |
| ATOM | 367 | CB | ALA | 853 | 26.791 | 30.769 | 49.729 | 1.00 | 20.85 |
| ATOM | 368 | C | ALA | 853 | 26.933 | 32.490 | 51.488 | 1.00 | 27.76 |
| ATOM | 369 | O | ALA | 853 | 27.118 | 33.472 | 50.757 | 1.00 | 28.81 |
| ATOM | 370 | N | PHE | 854 | 27.500 | 32.540 | 52.678 | 1.00 | 31.30 |
| ATOM | 371 | H | PHE | 854 | 27.465 | 31.730 | 53.217 | 1.00 | 25.00 |
| ATOM | 372 | CA | PHE | 854 | 28.258 | 33.735 | 53.145 | 1.00 | 31.68 |
| ATOM | 373 | CB | PHE | 854 | 28.598 | 33.625 | 54.609 | 1.00 | 32.24 |
| ATOM | 374 | CG | PHE | 854 | 28.898 | 34.959 | 55.213 | 1.00 | 32.72 |
| ATOM | 375 | CD1 | PHE | 854 | 30.212 | 35.380 | 55.514 | 1.00 | 33.25 |
| ATOM | 376 | CD2 | PHE | 854 | 27.818 | 35.781 | 55.604 | 1.00 | 32.60 |
| ATOM | 377 | CE1 | PHE | 854 | 30.418 | 36.609 | 56.208 | 1.00 | 33.17 |
| ATOM | 378 | CE2 | PHE | 854 | 28.030 | 36.975 | 56.283 | 1.00 | 28.50 |
| ATOM | 379 | CZ | PHE | 854 | 29.323 | 37.378 | 56.587 | 1.00 | 29.27 |
| ATOM | 380 | C | PHE | 854 | 29.536 | 34.099 | 52.483 | 1.00 | 31.44 |
| ATOM | 381 | O | PHE | 854 | 30.518 | 33.444 | 52.722 | 1.00 | 32.77 |
| ATOM | 382 | N | GLY | 855 | 29.612 | 35.044 | 51.593 | 1.00 | 31.32 |
| ATOM | 383 | H | GLY | 855 | 28.825 | 35.415 | 51.161 | 1.00 | 25.00 |
| ATOM | 384 | CA | GLY | 855 | 30.930 | 35.350 | 51.103 | 1.00 | 30.96 |
| ATOM | 385 | C | GLY | 855 | 31.449 | 34.385 | 50.060 | 1.00 | 34.61 |
| ATOM | 386 | O | GLY | 855 | 32.661 | 34.195 | 49.957 | 1.00 | 38.21 |
| ATOM | 387 | N | ILE | 856 | 30.588 | 33.778 | 49.221 | 1.00 | 37.06 |
| ATOM | 388 | H | ILE | 856 | 29.630 | 33.876 | 49.402 | 1.00 | 25.00 |
| ATOM | 389 | CA | ILE | 856 | 31.088 | 32.870 | 48.163 | 1.00 | 36.96 |
| ATOM | 390 | CB | ILE | 856 | 29.926 | 31.874 | 47.786 | 1.00 | 30.88 |
| ATOM | 391 | CG2 | ILE | 856 | 28.647 | 32.548 | 47.291 | 1.00 | 28.31 |
| ATOM | 392 | CG1 | ILE | 856 | 30.564 | 30.901 | 46.812 | 1.00 | 26.79 |
| ATOM | 393 | CD1 | ILE | 856 | 29.816 | 29.571 | 46.746 | 1.00 | 21.97 |
| ATOM | 394 | C | ILE | 856 | 31.560 | 33.803 | 47.042 | 1.00 | 43.13 |
| ATOM | 395 | O | ILE | 856 | 32.668 | 33.696 | 46.514 | 1.00 | 43.68 |
| ATOM | 396 | N | ASP | 857 | 30.723 | 34.802 | 46.731 | 1.00 | 48.61 |
| ATOM | 397 | H | ASP | 857 | 29.884 | 34.769 | 47.218 | 1.00 | 25.00 |
| ATOM | 398 | CA | ASP | 857 | 30.873 | 35.867 | 45.727 | 1.00 | 52.93 |
| ATOM | 399 | CB | ASP | 857 | 29.474 | 36.453 | 45.534 | 1.00 | 53.63 |
| ATOM | 1 | CG | ASP | 857 | 28.790 | 36.985 | 46.864 | 1.00 | 55.45 |
| ATOM | 401 | OD1 | ASP | 857 | 27.862 | 37.810 | 46.702 | 1.00 | 57.26 |
| ATOM | 402 | OD2 | ASP | 857 | 29.123 | 36.571 | 48.009 | 1.00 | 49.77 |
| ATOM | 403 | C | ASP | 857 | 31.847 | 36.899 | 46.256 | 1.00 | 56.43 |
| ATOM | 404 | O | ASP | 857 | 31.368 | 37.883 | 46.783 | 1.00 | 56.51 |
| ATOM | 405 | N | LYS | 858 | 33.181 | 36.706 | 46.227 | 1.00 | 60.58 |
| ATOM | 406 | H | LYS | 858 | 33.458 | 35.858 | 45.843 | 1.00 | 25.00 |
| ATOM | 407 | CA | LYS | 858 | 34.292 | 37.601 | 46.749 | 1.00 | 63.09 |
| ATOM | 408 | CB | LYS | 858 | 35.310 | 37.824 | 45.615 | 1.00 | 67.42 |
| ATOM | 409 | CG | LYS | 858 | 34.777 | 37.995 | 44.130 | 1.00 | 73.34 |
| ATOM | 410 | CD | LYS | 858 | 35.559 | 37.025 | 43.167 | 1.00 | 76.93 |
| ATOM | 411 | CE | LYS | 858 | 37.144 | 37.179 | 43.208 | 1.00 | 80.28 |
| ATOM | 412 | NZ | LYS | 858 | 37.903 | 35.968 | 42.827 | 1.00 | 80.91 |
| ATOM | 413 | HZ1 | LYS | 858 | 37.482 | 35.573 | 41.964 | 1.00 | 25.00 |
| ATOM | 414 | HZ2 | LYS | 858 | 37.836 | 35.297 | 43.618 | 1.00 | 25.00 |
| ATOM | 415 | HZ3 | LYS | 858 | 38.902 | 36.226 | 42.683 | 1.00 | 25.00 |
| ATOM | 416 | C | LYS | 858 | 33.899 | 38.981 | 47.387 | 1.00 | 63.18 |
| ATOM | 417 | O | LYS | 858 | 34.070 | 40.147 | 46.933 | 1.00 | 65.03 |
| ATOM | 418 | N | THR | 859 | 33.428 | 38.754 | 48.618 | 1.00 | 58.52 |
| ATOM | 419 | H | THR | 859 | 33.436 | 37.823 | 48.929 | 1.00 | 25.00 |
| ATOM | 420 | CA | THR | 859 | 32.883 | 39.755 | 49.538 | 1.00 | 51.08 |
| ATOM | 421 | CB | THR | 859 | 31.356 | 40.030 | 49.112 | 1.00 | 54.10 |
| ATOM | 422 | OG1 | THR | 859 | 30.790 | 38.749 | 49.380 | 1.00 | 52.30 |
| ATOM | 423 | HG1 | THR | 859 | 29.888 | 38.719 | 49.071 | 1.00 | 25.00 |
| ATOM | 424 | CG2 | THR | 859 | 30.894 | 40.426 | 47.636 | 1.00 | 51.17 |
| ATOM | 425 | C | THR | 859 | 32.944 | 39.011 | 50.904 | 1.00 | 45.06 |
| ATOM | 426 | O | THR | 859 | 33.831 | 38.223 | 51.117 | 1.00 | 42.70 |
| ATOM | 427 | N | ALA | 860 | 32.015 | 39.304 | 51.808 | 1.00 | 40.95 |
| ATOM | 428 | H | ALA | 860 | 31.490 | 40.068 | 51.492 | 1.00 | 25.00 |
| ATOM | 429 | CA | ALA | 860 | 31.648 | 38.770 | 53.153 | 1.00 | 37.04 |
| ATOM | 430 | CB | ALA | 860 | 32.351 | 39.303 | 54.393 | 1.00 | 37.95 |
| ATOM | 431 | C | ALA | 860 | 30.211 | 39.321 | 53.285 | 1.00 | 36.01 |
| ATOM | 432 | O | ALA | 860 | 29.795 | 40.003 | 54.224 | 1.00 | 32.58 |
| ATOM | 433 | N | THR | 861 | 29.506 | 39.012 | 52.123 | 1.00 | 35.15 |
| ATOM | 434 | H | THR | 861 | 30.142 | 38.620 | 51.482 | 1.00 | 25.00 |
| ATOM | 435 | CA | THR | 861 | 28.108 | 39.192 | 51.588 | 1.00 | 32.22 |
| ATOM | 436 | CB | THR | 861 | 28.089 | 39.716 | 50.124 | 1.00 | 32.71 |
| ATOM | 437 | OG1 | THR | 861 | 28.792 | 40.923 | 49.950 | 1.00 | 31.34 |
| ATOM | 438 | HG1 | THR | 861 | 28.805 | 41.192 | 49.023 | 1.00 | 25.00 |
| ATOM | 439 | CG2 | THR | 861 | 26.700 | 40.006 | 49.731 | 1.00 | 34.91 |
| ATOM | 440 | C | THR | 861 | 27.309 | 37.847 | 51.478 | 1.00 | 33.09 |
| ATOM | 441 | O | THR | 861 | 27.712 | 36.950 | 50.703 | 1.00 | 33.85 |
| ATOM | 442 | N | CYS | 862 | 26.237 | 37.598 | 52.214 | 1.00 | 31.97 |
| ATOM | 443 | H | CYS | 862 | 26.033 | 38.215 | 52.926 | 1.00 | 25.00 |
| ATOM | 444 | CA | CYS | 862 | 25.456 | 36.382 | 52.007 | 1.00 | 32.00 |
| ATOM | 445 | CB | CYS | 862 | 24.300 | 36.268 | 52.912 | 1.00 | 32.00 |
| ATOM | 446 | SG | CYS | 862 | 23.731 | 34.580 | 53.379 | 1.00 | 41.25 |
| ATOM | 447 | C | CYS | 862 | 24.867 | 36.473 | 50.612 | 1.00 | 33.60 |
| ATOM | 448 | O | CYS | 862 | 24.528 | 37.518 | 50.111 | 1.00 | 35.06 |
| ATOM | 449 | N | ARG | 863 | 24.740 | 35.396 | 49.898 | 1.00 | 35.34 |
| ATOM | 450 | H | ARG | 863 | 25.028 | 34.589 | 50.347 | 1.00 | 25.00 |
| ATOM | 451 | CA | ARG | 863 | 24.228 | 35.271 | 48.525 | 1.00 | 33.68 |
| ATOM | 452 | CB | ARG | 863 | 25.561 | 35.485 | 47.866 | 1.00 | 30.81 |
| ATOM | 453 | CG | ARG | 863 | 25.874 | 34.493 | 46.850 | 1.00 | 33.30 |
| ATOM | 454 | CD | ARG | 863 | 25.126 | 34.891 | 45.637 | 1.00 | 34.11 |
| ATOM | 455 | NE | ARG | 863 | 26.110 | 34.939 | 44.532 | 1.00 | 36.71 |
| ATOM | 456 | HE | ARG | 863 | 27.042 | 35.203 | 44.716 | 1.00 | 25.00 |
| ATOM | 457 | CZ | ARG | 863 | 25.753 | 34.752 | 43.253 | 1.00 | 38.78 |
| ATOM | 458 | NH1 | ARG | 863 | 26.716 | 34.875 | 42.317 | 1.00 | 38.58 |
| ATOM | 459 | HH11 | ARG | 863 | 27.635 | 35.131 | 42.615 | 1.00 | 25.00 |
| ATOM | 460 | HH12 | ARG | 863 | 26.521 | 34.735 | 41.344 | 1.00 | 25.00 |
| ATOM | 461 | NH2 | ARG | 863 | 24.504 | 34.313 | 42.947 | 1.00 | 42.39 |
| ATOM | 462 | HH21 | ARG | 863 | 23.884 | 34.117 | 43.707 | 1.00 | 25.00 |
| ATOM | 463 | HH22 | ARG | 863 | 24.250 | 34.134 | 42.004 | 1.00 | 25.00 |
| ATOM | 464 | C | ARG | 863 | 23.418 | 33.889 | 48.319 | 1.00 | 35.03 |
| ATOM | 465 | O | ARG | 863 | 23.820 | 32.820 | 48.796 | 1.00 | 37.13 |
| ATOM | 466 | N | THR | 864 | 22.183 | 33.804 | 47.777 | 1.00 | 33.98 |
| ATOM | 467 | H | THR | 864 | 21.750 | 34.639 | 47.484 | 1.00 | 25.00 |
| ATOM | 468 | CA | THR | 864 | 21.467 | 32.529 | 47.531 | 1.00 | 28.77 |
| ATOM | 469 | CB | THR | 864 | 20.066 | 32.799 | 47.261 | 1.00 | 28.00 |
| ATOM | 470 | OG1 | THR | 864 | 19.713 | 33.584 | 48.410 | 1.00 | 32.63 |
| ATOM | 471 | HG1 | THR | 864 | 18.809 | 33.884 | 48.305 | 1.00 | 25.00 |
| ATOM | 472 | CG2 | THR | 864 | 19.054 | 31.712 | 47.480 | 1.00 | 24.83 |
| ATOM | 473 | C | THR | 864 | 22.086 | 31.900 | 46.344 | 1.00 | 29.70 |
| ATOM | 474 | O | THR | 864 | 22.479 | 32.481 | 45.320 | 1.00 | 31.09 |
| ATOM | 475 | N | VAL | 865 | 22.462 | 30.682 | 46.575 | 1.00 | 28.93 |
| ATOM | 476 | H | VAL | 865 | 22.344 | 30.297 | 47.462 | 1.00 | 25.00 |
| ATOM | 477 | CA | VAL | 865 | 23.035 | 29.918 | 45.507 | 1.00 | 25.47 |
| ATOM | 478 | CB | VAL | 865 | 24.483 | 29.602 | 45.878 | 1.00 | 26.31 |
| ATOM | 479 | CG1 | VAL | 865 | 25.288 | 30.889 | 46.170 | 1.00 | 22.62 |
| ATOM | 480 | CG2 | VAL | 865 | 24.470 | 28.690 | 47.091 | 1.00 | 23.92 |
| ATOM | 481 | C | VAL | 865 | 22.174 | 28.645 | 45.381 | 1.00 | 24.99 |
| ATOM | 482 | O | VAL | 865 | 21.361 | 28.342 | 46.272 | 1.00 | 22.02 |
| ATOM | 483 | N | ALA | 866 | 22.285 | 27.915 | 44.254 | 1.00 | 22.82 |
| ATOM | 484 | H | ALA | 866 | 22.917 | 28.218 | 43.571 | 1.00 | 25.00 |
| ATOM | 485 | CA | ALA | 866 | 21.575 | 26.646 | 44.118 | 1.00 | 22.19 |
| ATOM | 486 | CB | ALA | 866 | 20.921 | 26.610 | 42.816 | 1.00 | 21.17 |
| ATOM | 487 | C | ALA | 866 | 22.645 | 25.521 | 44.160 | 1.00 | 23.74 |
| ATOM | 488 | O | ALA | 866 | 23.675 | 25.534 | 43.456 | 1.00 | 22.51 |
| ATOM | 489 | N | VAL | 867 | 22.555 | 24.537 | 45.028 | 1.00 | 23.89 |
| ATOM | 490 | H | VAL | 867 | 21.790 | 24.478 | 45.588 | 1.00 | 25.00 |
| ATOM | 491 | CA | VAL | 867 | 23.577 | 23.501 | 45.034 | 1.00 | 22.65 |
| ATOM | 492 | CB | VAL | 867 | 24.065 | 23.547 | 46.509 | 1.00 | 22.46 |
| ATOM | 493 | CG1 | VAL | 867 | 22.924 | 23.454 | 47.468 | 1.00 | 22.54 |
| ATOM | 494 | CG2 | VAL | 867 | 25.154 | 22.494 | 46.717 | 1.00 | 18.49 |
| ATOM | 495 | C | VAL | 867 | 23.161 | 22.129 | 44.468 | 1.00 | 24.35 |
| ATOM | 496 | O | VAL | 867 | 22.080 | 21.587 | 44.746 | 1.00 | 27.36 |
| ATOM | 497 | N | LYS | 868 | 23.896 | 21.608 | 43.471 | 1.00 | 23.54 |
| ATOM | 498 | H | LYS | 868 | 24.640 | 22.103 | 43.076 | 1.00 | 25.00 |
| ATOM | 499 | CA | LYS | 868 | 23.547 | 20.269 | 42.988 | 1.00 | 22.47 |
| ATOM | 500 | CB | LYS | 868 | 23.865 | 20.024 | 41.456 | 1.00 | 22.29 |
| ATOM | 501 | CG | LYS | 868 | 23.318 | 18.648 | 40.906 | 1.00 | 22.16 |
| ATOM | 502 | CD | LYS | 868 | 23.922 | 18.296 | 39.582 | 1.00 | 18.73 |
| ATOM | 503 | CE | LYS | 868 | 23.063 | 18.886 | 38.497 | 1.00 | 19.86 |
| ATOM | 504 | NZ | LYS | 868 | 23.692 | 18.694 | 37.181 | 1.00 | 21.99 |
| ATOM | 505 | HZ1 | LYS | 868 | 23.976 | 17.703 | 37.078 | 1.00 | 25.00 |
| ATOM | 506 | HZ2 | LYS | 868 | 24.529 | 19.290 | 37.125 | 1.00 | 25.00 |
| ATOM | 507 | HZ3 | LYS | 868 | 23.037 | 18.939 | 36.407 | 1.00 | 25.00 |
| ATOM | 508 | C | LYS | 868 | 24.432 | 19.263 | 43.794 | 1.00 | 22.77 |
| ATOM | 509 | O | LYS | 868 | 25.626 | 19.378 | 43.956 | 1.00 | 21.59 |
| ATOM | 510 | N | MET | 869 | 23.871 | 18.216 | 44.296 | 1.00 | 23.96 |
| ATOM | 511 | H | MET | 869 | 22.890 | 18.165 | 44.181 | 1.00 | 25.00 |
| ATOM | 512 | CA | MET | 869 | 24.612 | 17.140 | 44.973 | 1.00 | 25.30 |
| ATOM | 513 | CB | MET | 869 | 24.771 | 17.520 | 46.366 | 1.00 | 25.88 |
| ATOM | 514 | CG | MET | 869 | 23.410 | 17.744 | 46.843 | 1.00 | 29.28 |
| ATOM | 515 | SD | MET | 869 | 23.467 | 18.114 | 48.516 | 1.00 | 37.88 |
| ATOM | 516 | CE | MET | 869 | 21.901 | 18.879 | 48.477 | 1.00 | 36.24 |
| ATOM | 517 | C | MET | 869 | 23.797 | 15.814 | 44.854 | 1.00 | 24.97 |
| ATOM | 518 | O | MET | 869 | 22.658 | 15.763 | 44.390 | 1.00 | 25.57 |
| ATOM | 519 | N | LEU | 870 | 24.340 | 14.684 | 45.258 | 1.00 | 24.82 |
| ATOM | 520 | H | LEU | 870 | 25.241 | 14.743 | 45.621 | 1.00 | 25.00 |
| ATOM | 521 | CA | LEU | 870 | 23.664 | 13.438 | 45.241 | 1.00 | 22.92 |
| ATOM | 522 | CB | LEU | 870 | 24.662 | 12.302 | 45.185 | 1.00 | 23.08 |
| ATOM | 523 | CG | LEU | 870 | 25.551 | 12.307 | 43.996 | 1.00 | 17.43 |
| ATOM | 524 | CD1 | LEU | 870 | 26.509 | 11.168 | 44.437 | 1.00 | 15.50 |
| ATOM | 525 | CD2 | LEU | 870 | 24.768 | 12.436 | 42.624 | 1.00 | 14.61 |
| ATOM | 526 | C | LEU | 870 | 22.773 | 13.190 | 46.393 | 1.00 | 23.17 |
| ATOM | 527 | O | LEU | 870 | 22.782 | 13.671 | 47.477 | 1.00 | 23.91 |
| ATOM | 528 | N | LYS | 871 | 21.825 | 12.409 | 46.090 | 1.00 | 29.24 |
| ATOM | 529 | H | LYS | 871 | 21.737 | 12.174 | 45.143 | 1.00 | 25.00 |
| ATOM | 530 | CA | LYS | 871 | 20.786 | 11.947 | 47.024 | 1.00 | 32.95 |
| ATOM | 531 | CB | LYS | 871 | 19.473 | 12.110 | 46.185 | 1.00 | 30.68 |
| ATOM | 532 | CG | LYS | 871 | 18.507 | 12.796 | 47.073 | 1.00 | 27.85 |
| ATOM | 533 | CD | LYS | 871 | 17.356 | 12.847 | 46.200 | 1.00 | 29.55 |
| ATOM | 534 | CE | LYS | 871 | 16.716 | 11.487 | 46.090 | 1.00 | 31.75 |
| ATOM | 535 | NZ | LYS | 871 | 15.333 | 11.750 | 46.467 | 1.00 | 36.68 |
| ATOM | 536 | HZ1 | LYS | 871 | 15.013 | 12.621 | 45.995 | 1.00 | 25.00 |
| ATOM | 537 | HZ2 | LYS | 871 | 15.291 | 11.918 | 47.494 | 1.00 | 25.00 |
| ATOM | 538 | HZ3 | LYS | 871 | 14.718 | 10.961 | 46.179 | 1.00 | 25.00 |
| ATOM | 539 | C | LYS | 871 | 21.112 | 10.478 | 47.481 | 1.00 | 34.52 |
| ATOM | 540 | O | LYS | 871 | 21.991 | 9.876 | 46.852 | 1.00 | 32.40 |
| ATOM | 541 | N | GLU | 872 | 20.532 | 9.835 | 48.536 | 1.00 | 39.70 |
| ATOM | 542 | H | GLU | 872 | 19.912 | 10.304 | 49.131 | 1.00 | 25.00 |
| ATOM | 543 | CA | GLU | 872 | 21.001 | 8.440 | 48.739 | 1.00 | 45.47 |
| ATOM | 544 | CB | GLU | 872 | 20.716 | 7.894 | 50.181 | 1.00 | 47.67 |
| ATOM | 545 | CG | GLU | 872 | 22.232 | 7.310 | 50.703 | 1.00 | 53.03 |
| ATOM | 546 | CD | GLU | 872 | 23.645 | 7.043 | 49.842 | 1.00 | 53.05 |
| ATOM | 547 | OE1 | GLU | 872 | 23.740 | 6.324 | 48.816 | 1.00 | 51.88 |
| ATOM | 548 | OE2 | GLU | 872 | 24.726 | 7.497 | 50.277 | 1.00 | 51.56 |
| ATOM | 549 | C | GLU | 872 | 20.504 | 7.434 | 47.692 | 1.00 | 45.40 |
| ATOM | 550 | O | GLU | 872 | 19.643 | 7.702 | 46.865 | 1.00 | 48.81 |
| ATOM | 551 | N | GLY | 873 | 21.056 | 6.244 | 47.640 | 1.00 | 44.45 |
| ATOM | 552 | H | GLY | 873 | 21.717 | 5.968 | 48.311 | 1.00 | 25.00 |
| ATOM | 553 | CA | GLY | 873 | 20.649 | 5.315 | 46.564 | 1.00 | 43.91 |
| ATOM | 554 | C | GLY | 873 | 21.574 | 5.553 | 45.378 | 1.00 | 42.39 |
| ATOM | 555 | O | GLY | 873 | 21.625 | 4.851 | 44.356 | 1.00 | 41.91 |
| ATOM | 556 | N | ALA | 874 | 22.275 | 6.668 | 45.496 | 1.00 | 41.27 |
| ATOM | 557 | H | ALA | 874 | 22.120 | 7.240 | 46.262 | 1.00 | 25.00 |
| ATOM | 558 | CA | ALA | 874 | 23.263 | 7.029 | 44.513 | 1.00 | 38.10 |
| ATOM | 559 | CB | ALA | 874 | 23.509 | 8.533 | 44.557 | 1.00 | 36.92 |
| ATOM | 560 | C | ALA | 874 | 24.541 | 6.321 | 44.902 | 1.00 | 36.19 |
| ATOM | 561 | O | ALA | 874 | 24.782 | 5.983 | 46.065 | 1.00 | 35.35 |
| ATOM | 562 | N | THR | 875 | 25.463 | 6.254 | 43.961 | 1.00 | 35.33 |
| ATOM | 563 | H | THR | 875 | 25.280 | 6.598 | 43.066 | 1.00 | 25.00 |
| ATOM | 564 | CA | THR | 875 | 26.729 | 5.655 | 44.268 | 1.00 | 36.04 |
| ATOM | 565 | CB | THR | 875 | 26.854 | 4.336 | 43.620 | 1.00 | 39.40 |
| ATOM | 566 | OG1 | THR | 875 | 26.486 | 4.491 | 42.250 | 1.00 | 39.70 |
| ATOM | 567 | HG1 | THR | 875 | 26.659 | 3.654 | 41.805 | 1.00 | 25.00 |
| ATOM | 568 | CG2 | THR | 875 | 26.058 | 3.305 | 44.452 | 1.00 | 43.09 |
| ATOM | 569 | C | THR | 875 | 28.015 | 6.371 | 43.931 | 1.00 | 34.20 |
| ATOM | 570 | O | THR | 875 | 28.112 | 7.330 | 43.190 | 1.00 | 34.84 |
| ATOM | 571 | N | HIS | 876 | 29.135 | 5.824 | 44.352 | 1.00 | 29.85 |
| ATOM | 572 | H | HIS | 876 | 29.092 | 4.987 | 44.850 | 1.00 | 25.00 |
| ATOM | 573 | CA | HIS | 876 | 30.367 | 6.475 | 44.094 | 1.00 | 26.33 |
| ATOM | 574 | C | HIS | 876 | 30.549 | 6.750 | 42.694 | 1.00 | 25.95 |
| ATOM | 575 | O | HIS | 876 | 31.408 | 7.511 | 42.306 | 1.00 | 30.21 |
| ATOM | 576 | CB | HIS | 876 | 31.439 | 5.607 | 44.570 | 1.00 | 21.93 |
| ATOM | 577 | CC | HIS | 876 | 32.679 | 6.263 | 44.250 | 1.00 | 19.78 |
| ATOM | 578 | ND1 | HIS | 876 | 33.461 | 5.877 | 43.217 | 1.00 | 21.43 |
| ATOM | 579 | HD1 | HIS | 876 | 33.241 | 5.291 | 42.456 | 1.00 | 25.00 |
| ATOM | 580 | CD2 | HIS | 876 | 33.197 | 7.327 | 44.961 | 1.00 | 18.21 |
| ATOM | 581 | NE2 | HIS | 876 | 34.344 | 7.600 | 44.345 | 1.00 | 17.17 |
| ATOM | 582 | CE1 | HIS | 876 | 34.511 | 6.715 | 43.285 | 1.00 | 20.91 |
| ATOM | 583 | N | SER | 877 | 29.886 | 6.074 | 41.851 | 1.00 | 24.60 |
| ATOM | 584 | H | SER | 877 | 29.229 | 5.413 | 42.101 | 1.00 | 25.00 |
| ATOM | 585 | CA | SER | 877 | 30.156 | 6.350 | 40.427 | 1.00 | 29.07 |
| ATOM | 586 | CB | SER | 877 | 29.974 | 4.993 | 39.610 | 1.00 | 29.02 |
| ATOM | 587 | OG | SER | 877 | 29.051 | 4.182 | 40.332 | 1.00 | 39.53 |
| ATOM | 588 | HG | SER | 877 | 28.865 | 3.373 | 39.842 | 1.00 | 25.00 |
| ATOM | 589 | C | SER | 877 | 29.338 | 7.538 | 39.905 | 1.00 | 26.02 |
| ATOM | 590 | O | SER | 877 | 29.696 | 8.168 | 38.950 | 1.00 | 24.46 |
| ATOM | 591 | N | GLU | 878 | 28.215 | 7.820 | 40.522 | 1.00 | 25.87 |
| ATOM | 592 | H | GLU | 878 | 27.938 | 7.236 | 41.251 | 1.00 | 25.00 |
| ATOM | 593 | CA | GLU | 878 | 27.387 | 8.951 | 40.251 | 1.00 | 27.80 |
| ATOM | 594 | CB | GLU | 878 | 26.163 | 8.948 | 41.055 | 1.00 | 31.22 |
| ATOM | 595 | CG | GLU | 878 | 24.879 | 8.758 | 40.289 | 1.00 | 34.27 |
| ATOM | 596 | CD | GLU | 878 | 24.385 | 7.381 | 40.498 | 1.00 | 36.68 |
| ATOM | 597 | OE1 | GLU | 878 | 23.681 | 7.185 | 41.497 | 1.00 | 38.24 |
| ATOM | 598 | OE2 | GLU | 878 | 24.689 | 6.563 | 39.627 | 1.00 | 37.44 |
| ATOM | 599 | C | GLU | 878 | 28.283 | 9.955 | 40.858 | 1.00 | 29.91 |
| ATOM | 600 | O | GLU | 878 | 28.784 | 10.872 | 40.255 | 1.00 | 34.08 |
| ATOM | 601 | N | HIS | 879 | 28.666 | 9.748 | 42.083 | 1.00 | 30.08 |
| ATOM | 602 | H | HIS | 879 | 28.362 | 8.940 | 42.505 | 1.00 | 25.00 |
| ATOM | 603 | CA | HIS | 879 | 29.599 | 10.617 | 42.768 | 1.00 | 27.58 |
| ATOM | 604 | C | HIS | 879 | 30.721 | 11.030 | 41.815 | 1.00 | 27.41 |
| ATOM | 605 | O | HIS | 879 | 30.935 | 12.204 | 41.516 | 1.00 | 28.47 |
| ATOM | 606 | CB | HIS | 879 | 30.056 | 9.787 | 43.969 | 1.00 | 23.32 |
| ATOM | 607 | CG | HIS | 879 | 31.139 | 10.596 | 44.526 | 1.00 | 16.88 |
| ATOM | 608 | ND1 | HIS | 879 | 32.380 | 10.799 | 44.108 | 1.00 | 14.29 |
| ATOM | 609 | HD1 | HIS | 879 | 32.735 | 10.299 | 43.387 | 1.00 | 25.00 |
| ATOM | 610 | CD2 | HIS | 879 | 31.008 | 11.421 | 45.624 | 1.00 | 14.24 |
| ATOM | 611 | NE2 | HIS | 879 | 32.112 | 12.101 | 45.869 | 1.00 | 10.81 |
| ATOM | 612 | CE1 | HIS | 879 | 33.005 | 11.737 | 44.923 | 1.00 | 9.68 |
| ATOM | 613 | N | ARG | 880 | 31.331 | 10.032 | 41.200 | 1.00 | 29.59 |
| ATOM | 614 | H | ARG | 880 | 31.053 | 9.128 | 41.442 | 1.00 | 25.00 |
| ATOM | 615 | CA | ARG | 880 | 32.458 | 10.231 | 40.257 | 1.00 | 28.14 |
| ATOM | 616 | CB | ARG | 880 | 32.848 | 8.923 | 39.675 | 1.00 | 28.01 |
| ATOM | 617 | CG | ARG | 880 | 34.115 | 8.610 | 40.410 | 1.00 | 36.37 |
| ATOM | 618 | CD | ARG | 880 | 35.078 | 7.481 | 39.839 | 1.00 | 43.98 |
| ATOM | 619 | NE | ARG | 880 | 34.359 | 6.192 | 39.917 | 1.00 | 48.21 |
| ATOM | 620 | HE | ARG | 880 | 33.454 | 6.180 | 39.533 | 1.00 | 25.00 |
| ATOM | 621 | CZ | ARG | 880 | 34.786 | 4.990 | 40.398 | 1.00 | 49.87 |
| ATOM | 622 | NH1 | ARG | 880 | 33.860 | 3.976 | 40.354 | 1.00 | 49.23 |
| ATOM | 623 | HH11 | ARG | 880 | 32.959 | 4.167 | 39.954 | 1.00 | 25.00 |
| ATOM | 624 | HH12 | ARG | 880 | 34.070 | 3.048 | 40.676 | 1.00 | 25.00 |
| ATOM | 625 | NH2 | ARG | 880 | 36.034 | 4.771 | 40.843 | 1.00 | 45.50 |
| ATOM | 626 | HH21 | ARG | 880 | 36.674 | 5.532 | 40.847 | 1.00 | 25.00 |
| ATOM | 627 | HH22 | ARG | 880 | 36.285 | 3.868 | 41.204 | 1.00 | 25.00 |
| ATOM | 628 | C | ARG | 880 | 32.176 | 11.202 | 39.112 | 1.00 | 27.17 |
| ATOM | 629 | O | ARG | 880 | 32.968 | 12.092 | 38.781 | 1.00 | 25.36 |
| ATOM | 630 | N | ALA | 881 | 30.964 | 10.997 | 38.569 | 1.00 | 23.27 |
| ATOM | 631 | H | ALA | 881 | 30.434 | 10.245 | 38.878 | 1.00 | 25.00 |
| ATOM | 632 | CA | ALA | 881 | 30.321 | 11.710 | 37.466 | 1.00 | 21.44 |
| ATOM | 633 | CB | ALA | 881 | 28.965 | 11.005 | 37.161 | 1.00 | 12.68 |
| ATOM | 634 | C | ALA | 881 | 30.121 | 13.177 | 37.801 | 1.00 | 22.73 |
| ATOM | 635 | O | ALA | 881 | 30.354 | 13.976 | 36.912 | 1.00 | 24.07 |
| ATOM | 636 | N | LEU | 882 | 29.608 | 13.546 | 39.039 | 1.00 | 25.18 |
| ATOM | 637 | H | LEU | 882 | 29.433 | 12.780 | 39.650 | 1.00 | 25.00 |
| ATOM | 638 | CA | LEU | 882 | 29.387 | 14.918 | 39.645 | 1.00 | 22.45 |
| ATOM | 639 | CB | LEU | 882 | 28.796 | 14.859 | 41.027 | 1.00 | 18.14 |
| ATOM | 640 | CG | LEU | 882 | 28.386 | 16.205 | 41.680 | 1.00 | 15.70 |
| ATOM | 641 | CD1 | LEU | 882 | 27.464 | 16.962 | 40.722 | 1.00 | 14.80 |
| ATOM | 642 | CD2 | LEU | 882 | 27.654 | 15.989 | 43.008 | 1.00 | 5.10 |
| ATOM | 643 | C | LEU | 882 | 30.789 | 15.445 | 39.791 | 1.00 | 27.06 |
| ATOM | 644 | O | LEU | 882 | 31.044 | 16.547 | 39.301 | 1.00 | 30.55 |
| ATOM | 645 | N | MET | 883 | 31.800 | 14.722 | 40.378 | 1.00 | 28.55 |
| ATOM | 646 | H | MET | 883 | 31.696 | 13.803 | 40.687 | 1.00 | 25.00 |
| ATOM | 647 | CA | MET | 883 | 33.072 | 15.407 | 40.357 | 1.00 | 29.16 |
| ATOM | 648 | CB | MET | 883 | 34.076 | 14.555 | 40.990 | 1.00 | 28.74 |
| ATOM | 649 | CG | MET | 883 | 35.488 | 15.108 | 40.818 | 1.00 | 29.21 |
| ATOM | 650 | SD | MET | 883 | 35.932 | 16.692 | 41.586 | 1.00 | 33.06 |
| ATOM | 651 | CE | MET | 883 | 35.199 | 16.308 | 43.117 | 1.00 | 29.34 |
| ATOM | 652 | C | MET | 883 | 33.507 | 15.775 | 38.944 | 1.00 | 30.91 |
| ATOM | 653 | O | MET | 883 | 34.174 | 16.797 | 38.826 | 1.00 | 34.96 |
| ATOM | 654 | N | SER | 884 | 33.299 | 15.059 | 37.825 | 1.00 | 31.24 |
| ATOM | 655 | H | SER | 884 | 32.818 | 14.194 | 37.815 | 1.00 | 25.00 |
| ATOM | 656 | CA | SER | 884 | 33.800 | 15.727 | 36.616 | 1.00 | 31.09 |
| ATOM | 657 | CB | SER | 884 | 34.483 | 14.802 | 35.545 | 1.00 | 33.86 |
| ATOM | 658 | OG | SER | 884 | 34.071 | 13.492 | 35.491 | 1.00 | 35.66 |
| ATOM | 659 | HG | SER | 884 | 34.577 | 13.020 | 34.834 | 1.00 | 25.00 |
| ATOM | 660 | C | SER | 884 | 32.728 | 16.605 | 35.919 | 1.00 | 29.32 |
| ATOM | 661 | O | SER | 884 | 33.058 | 17.306 | 34.960 | 1.00 | 28.44 |
| ATOM | 662 | N | GLU | 885 | 31.444 | 16.674 | 36.334 | 1.00 | 28.74 |
| ATOM | 663 | H | GLU | 885 | 31.151 | 16.061 | 37.047 | 1.00 | 25.00 |
| ATOM | 664 | CA | GLU | 885 | 30.508 | 17.650 | 35.735 | 1.00 | 28.12 |
| ATOM | 665 | CB | GLU | 885 | 29.153 | 17.392 | 36.314 | 1.00 | 26.99 |
| ATOM | 666 | CG | GLU | 885 | 28.141 | 18.077 | 35.494 | 1.00 | 29.08 |
| ATOM | 667 | CD | GLU | 885 | 26.893 | 18.125 | 36.295 | 1.00 | 34.90 |
| ATOM | 668 | OE1 | GLU | 885 | 26.067 | 17.199 | 36.297 | 1.00 | 40.41 |
| ATOM | 669 | OE2 | GLU | 885 | 26.714 | 19.135 | 36.947 | 1.00 | 35.33 |
| ATOM | 670 | C | GLU | 885 | 31.142 | 19.043 | 36.250 | 1.00 | 29.53 |
| ATOM | 671 | O | GLU | 885 | 31.262 | 20.029 | 35.524 | 1.00 | 30.47 |
| ATOM | 672 | N | LEU | 886 | 31.627 | 19.132 | 37.491 | 1.00 | 26.35 |
| ATOM | 673 | H | LEU | 886 | 31.592 | 18.286 | 37.985 | 1.00 | 25.00 |
| ATOM | 674 | CA | LEU | 886 | 32.250 | 20.296 | 38.075 | 1.00 | 24.63 |
| ATOM | 675 | CB | LEU | 886 | 32.659 | 19.925 | 39.402 | 1.00 | 21.61 |
| ATOM | 676 | CG | LEU | 886 | 33.609 | 20.869 | 40.050 | 1.00 | 22.79 |
| ATOM | 677 | CD1 | LEU | 886 | 32.983 | 22.256 | 39.971 | 1.00 | 20.11 |
| ATOM | 678 | CD2 | LEU | 886 | 33.980 | 20.346 | 41.472 | 1.00 | 21.12 |
| ATOM | 679 | C | LEU | 886 | 33.440 | 20.699 | 37.317 | 1.00 | 27.59 |
| ATOM | 680 | O | LEU | 886 | 33.787 | 21.878 | 37.201 | 1.00 | 29.02 |
| ATOM | 681 | N | LYS | 887 | 34.190 | 19.697 | 36.836 | 1.00 | 30.52 |
| ATOM | 682 | H | LYS | 887 | 33.860 | 18.780 | 36.902 | 1.00 | 25.00 |
| ATOM | 683 | CA | LYS | 887 | 35.472 | 19.962 | 36.107 | 1.00 | 31.15 |
| ATOM | 684 | CB | LYS | 887 | 36.459 | 18.716 | 36.137 | 1.00 | 32.61 |
| ATOM | 685 | CG | LYS | 887 | 37.441 | 18.290 | 37.268 | 1.00 | 31.87 |
| ATOM | 686 | CD | LYS | 887 | 38.106 | 16.927 | 36.721 | 1.00 | 38.00 |
| ATOM | 687 | CE | LYS | 887 | 39.178 | 16.157 | 37.666 | 1.00 | 39.62 |
| ATOM | 688 | NZ | LYS | 887 | 40.154 | 15.314 | 36.914 | 1.00 | 37.20 |
| ATOM | 689 | HZ1 | LYS | 887 | 40.410 | 15.764 | 36.011 | 1.00 | 25.00 |
| ATOM | 690 | HZ2 | LYS | 887 | 39.693 | 14.405 | 36.691 | 1.00 | 25.00 |
| ATOM | 691 | HZ3 | LYS | 887 | 41.025 | 15.160 | 37.461 | 1.00 | 25.00 |
| ATOM | 692 | C | LYS | 887 | 35.250 | 20.351 | 34.657 | 1.00 | 30.98 |
| ATOM | 693 | O | LYS | 887 | 36.094 | 20.990 | 34.013 | 1.00 | 29.50 |
| ATOM | 694 | N | ILE | 888 | 34.199 | 19.763 | 34.090 | 1.00 | 31.99 |
| ATOM | 695 | H | ILE | 888 | 33.581 | 19.238 | 34.639 | 1.00 | 25.00 |
| ATOM | 696 | CA | ILE | 888 | 33.880 | 20.127 | 32.709 | 1.00 | 33.48 |
| ATOM | 697 | CB | ILE | 888 | 32.823 | 19.075 | 32.095 | 1.00 | 35.28 |
| ATOM | 698 | CG2 | ILE | 888 | 31.529 | 19.037 | 32.794 | 1.00 | 36.80 |
| ATOM | 699 | CG1 | ILE | 888 | 32.379 | 19.570 | 30.687 | 1.00 | 36.48 |
| ATOM | 700 | CD1 | ILE | 888 | 33.552 | 19.257 | 29.802 | 1.00 | 42.46 |
| ATOM | 701 | C | ILE | 888 | 33.345 | 21.613 | 32.844 | 1.00 | 31.59 |
| ATOM | 702 | O | ILE | 888 | 33.893 | 22.551 | 32.310 | 1.00 | 30.89 |
| ATOM | 703 | N | LEU | 889 | 32.453 | 21.878 | 33.777 | 1.00 | 29.99 |
| ATOM | 704 | H | LEU | 889 | 32.129 | 21.144 | 34.341 | 1.00 | 25.00 |
| ATOM | 705 | CA | LEU | 889 | 31.906 | 23.202 | 34.029 | 1.00 | 26.04 |
| ATOM | 706 | CB | LEU | 889 | 30.977 | 23.019 | 35.238 | 1.00 | 18.85 |
| ATOM | 707 | CG | LEU | 889 | 29.600 | 22.787 | 35.175 | 1.00 | 13.38 |
| ATOM | 708 | CD1 | LEU | 889 | 29.128 | 22.810 | 36.542 | 1.00 | 10.99 |
| ATOM | 709 | CD2 | LEU | 889 | 28.884 | 23.933 | 34.474 | 1.00 | 20.29 |
| ATOM | 710 | C | LEU | 889 | 33.087 | 24.179 | 34.287 | 1.00 | 29.06 |
| ATOM | 711 | O | LEU | 889 | 33.065 | 25.334 | 33.888 | 1.00 | 30.19 |
| ATOM | 712 | N | ILE | 890 | 34.174 | 23.766 | 34.943 | 1.00 | 31.75 |
| ATOM | 713 | H | ILE | 890 | 34.162 | 22.861 | 35.279 | 1.00 | 25.00 |
| ATOM | 714 | CA | ILE | 890 | 35.285 | 24.702 | 35.222 | 1.00 | 30.73 |
| ATOM | 715 | CB | ILE | 890 | 36.333 | 24.131 | 36.294 | 1.00 | 30.59 |
| ATOM | 716 | CG2 | ILE | 890 | 37.483 | 25.185 | 36.501 | 1.00 | 27.96 |
| ATOM | 717 | CG1 | ILE | 890 | 35.733 | 23.929 | 37.737 | 1.00 | 26.44 |
| ATOM | 718 | CD1 | ILE | 890 | 36.944 | 23.291 | 38.468 | 1.00 | 25.74 |
| ATOM | 719 | C | ILE | 890 | 35.969 | 24.940 | 33.927 | 1.00 | 32.11 |
| ATOM | 720 | O | ILE | 890 | 36.615 | 25.963 | 33.691 | 1.00 | 32.66 |
| ATOM | 721 | N | HIS | 891 | 35.959 | 23.937 | 33.061 | 1.00 | 32.83 |
| ATOM | 722 | H | HIS | 891 | 35.628 | 23.049 | 33.296 | 1.00 | 25.00 |
| ATOM | 723 | CA | HIS | 891 | 36.605 | 24.093 | 31.744 | 1.00 | 33.25 |
| ATOM | 724 | C | HIS | 891 | 35.830 | 24.908 | 30.639 | 1.00 | 30.46 |
| ATOM | 725 | O | HIS | 891 | 36.431 | 25.625 | 29.840 | 1.00 | 29.67 |
| ATOM | 726 | CB | HIS | 891 | 36.964 | 22.673 | 31.223 | 1.00 | 35.75 |
| ATOM | 727 | CG | HIS | 891 | 37.293 | 22.757 | 29.769 | 1.00 | 41.04 |
| ATOM | 728 | ND1 | HIS | 891 | 38.560 | 23.076 | 29.323 | 1.00 | 39.93 |
| ATOM | 729 | CE1 | HIS | 891 | 38.385 | 22.922 | 27.948 | 1.00 | 44.65 |
| ATOM | 730 | CD2 | HIS | 891 | 36.364 | 22.436 | 28.686 | 1.00 | 41.99 |
| ATOM | 731 | NE2 | HIS | 891 | 37.077 | 22.546 | 27.562 | 1.00 | 43.11 |
| ATOM | 732 | HE2 | HIS | 891 | 36.784 | 22.465 | 26.653 | 1.00 | 25.00 |
| ATOM | 733 | N | ILE | 892 | 34.554 | 24.627 | 30.460 | 1.00 | 27.41 |
| ATOM | 734 | H | ILE | 892 | 34.156 | 24.000 | 31.108 | 1.00 | 25.00 |
| ATOM | 735 | CA | ILE | 892 | 33.710 | 25.358 | 29.498 | 1.00 | 28.16 |
| ATOM | 736 | CB | ILE | 892 | 32.299 | 24.999 | 29.814 | 1.00 | 23.20 |
| ATOM | 737 | CG2 | ILE | 892 | 31.384 | 25.790 | 28.885 | 1.00 | 21.75 |
| ATOM | 738 | CG1 | ILE | 892 | 32.130 | 23.426 | 29.695 | 1.00 | 21.79 |
| ATOM | 739 | CD1 | ILE | 892 | 30.668 | 22.852 | 30.000 | 1.00 | 12.96 |
| ATOM | 740 | C | ILE | 892 | 34.000 | 26.897 | 29.771 | 1.00 | 29.86 |
| ATOM | 741 | O | ILE | 892 | 34.478 | 27.672 | 28.944 | 1.00 | 31.34 |
| ATOM | 742 | N | GLY | 893 | 33.751 | 27.399 | 30.987 | 1.00 | 30.73 |
| ATOM | 743 | H | GLY | 893 | 33.440 | 26.774 | 31.678 | 1.00 | 25.00 |
| ATOM | 744 | CA | GLY | 893 | 34.056 | 28.808 | 31.384 | 1.00 | 28.58 |
| ATOM | 745 | C | GLY | 893 | 32.863 | 29.706 | 31.647 | 1.00 | 30.62 |
| ATOM | 746 | O | GLY | 893 | 31.707 | 29.271 | 31.780 | 1.00 | 30.86 |
| ATOM | 747 | N | HIS | 894 | 33.123 | 31.002 | 31.830 | 1.00 | 28.79 |
| ATOM | 748 | H | HIS | 894 | 34.028 | 31.359 | 31.785 | 1.00 | 25.00 |
| ATOM | 749 | CA | HIS | 894 | 31.975 | 31.898 | 32.070 | 1.00 | 27.99 |
| ATOM | 750 | C | HIS | 894 | 31.192 | 32.336 | 30.859 | 1.00 | 25.65 |
| ATOM | 751 | O | HIS | 894 | 31.760 | 32.564 | 29.792 | 1.00 | 27.42 |
| ATOM | 752 | CB | HIS | 894 | 32.454 | 33.161 | 32.809 | 1.00 | 27.91 |
| ATOM | 753 | CG | HIS | 894 | 31.325 | 34.076 | 33.138 | 1.00 | 28.10 |
| ATOM | 754 | ND1 | HIS | 894 | 30.953 | 35.225 | 32.431 | 1.00 | 24.54 |
| ATOM | 755 | CE1 | HIS | 894 | 29.865 | 35.720 | 33.132 | 1.00 | 24.46 |
| ATOM | 756 | CD2 | HIS | 894 | 30.471 | 33.894 | 34.237 | 1.00 | 27.92 |
| ATOM | 757 | NE2 | HIS | 894 | 29.565 | 34.912 | 34.229 | 1.00 | 28.64 |
| ATOM | 758 | HE2 | HIS | 894 | 28.835 | 35.049 | 34.858 | 1.00 | 25.00 |
| ATOM | 759 | N | HIS | 895 | 29.887 | 32.421 | 31.021 | 1.00 | 21.32 |
| ATOM | 760 | H | HIS | 895 | 29.484 | 32.005 | 31.833 | 1.00 | 25.00 |
| ATOM | 761 | CA | HIS | 895 | 29.059 | 32.932 | 29.966 | 1.00 | 16.61 |
| ATOM | 762 | C | HIS | 895 | 27.842 | 33.254 | 30.674 | 1.00 | 18.03 |
| ATOM | 763 | O | HIS | 895 | 27.481 | 32.675 | 31.697 | 1.00 | 18.61 |
| ATOM | 764 | CB | HIS | 895 | 28.761 | 31.988 | 28.797 | 1.00 | 14.85 |
| ATOM | 765 | CG | HIS | 895 | 28.106 | 32.699 | 27.627 | 1.00 | 12.22 |
| ATOM | 766 | ND1 | HIS | 895 | 26.849 | 33.249 | 27.590 | 1.00 | 12.84 |
| ATOM | 767 | CE1 | HIS | 895 | 26.622 | 33.926 | 26.412 | 1.00 | 15.46 |
| ATOM | 768 | CD2 | HIS | 895 | 28.639 | 33.085 | 26.415 | 1.00 | 10.38 |
| ATOM | 769 | NE2 | HIS | 895 | 27.756 | 33.816 | 25.686 | 1.00 | 11.92 |
| ATOM | 770 | HE2 | HIS | 895 | 27.889 | 34.159 | 24.786 | 1.00 | 25.00 |
| ATOM | 771 | N | LEU | 896 | 27.406 | 34.461 | 30.296 | 1.00 | 18.90 |
| ATOM | 772 | H | LEU | 896 | 27.882 | 34.965 | 29.608 | 1.00 | 25.00 |
| ATOM | 773 | CA | LEU | 896 | 26.200 | 34.991 | 30.883 | 1.00 | 19.38 |
| ATOM | 774 | CB | LEU | 896 | 25.802 | 36.265 | 30.179 | 1.00 | 20.38 |
| ATOM | 775 | CG | LEU | 896 | 24.400 | 36.931 | 30.544 | 1.00 | 23.00 |
| ATOM | 776 | CD1 | LEU | 896 | 24.567 | 37.741 | 31.769 | 1.00 | 22.60 |
| ATOM | 777 | CD2 | LEU | 896 | 23.927 | 38.058 | 29.612 | 1.00 | 23.89 |
| ATOM | 778 | C | LEU | 896 | 25.053 | 34.036 | 30.765 | 1.00 | 19.79 |
| ATOM | 779 | O | LEU | 896 | 24.084 | 34.021 | 31.548 | 1.00 | 22.07 |
| ATOM | 780 | N | ASN | 897 | 25.090 | 33.304 | 29.652 | 1.00 | 19.51 |
| ATOM | 781 | H | ASN | 897 | 25.796 | 33.467 | 29.026 | 1.00 | 25.00 |
| ATOM | 782 | CA | ASN | 897 | 23.999 | 32.362 | 29.311 | 1.00 | 20.37 |
| ATOM | 783 | CB | ASN | 897 | 23.671 | 32.733 | 27.856 | 1.00 | 23.52 |
| ATOM | 784 | CG | ASN | 897 | 22.986 | 34.179 | 27.826 | 1.00 | 19.30 |
| ATOM | 785 | OD1 | ASN | 897 | 23.408 | 35.072 | 27.086 | 1.00 | 15.81 |
| ATOM | 786 | ND2 | ASN | 897 | 21.955 | 34.425 | 28.600 | 1.00 | 9.84 |
| ATOM | 787 | HD21 | ASN | 897 | 21.607 | 33.723 | 29.174 | 1.00 | 25.00 |
| ATOM | 788 | HD22 | ASN | 897 | 21.522 | 35.302 | 28.585 | 1.00 | 25.00 |
| ATOM | 789 | C | ASN | 897 | 24.046 | 30.904 | 29.570 | 1.00 | 17.49 |
| ATOM | 790 | O | ASN | 897 | 23.126 | 30.178 | 29.305 | 1.00 | 15.04 |
| ATOM | 791 | N | VAL | 898 | 25.089 | 30.500 | 30.208 | 1.00 | 19.45 |
| ATOM | 792 | H | VAL | 898 | 25.824 | 31.123 | 30.152 | 1.00 | 25.00 |
| ATOM | 793 | CA | VAL | 898 | 25.230 | 29.139 | 30.762 | 1.00 | 20.42 |
| ATOM | 794 | CB | VAL | 898 | 26.497 | 28.448 | 30.295 | 1.00 | 17.94 |
| ATOM | 795 | CG1 | VAL | 898 | 26.406 | 28.346 | 28.813 | 1.00 | 17.47 |
| ATOM | 796 | CG2 | VAL | 898 | 27.686 | 29.255 | 30.258 | 1.00 | 27.90 |
| ATOM | 797 | C | VAL | 898 | 25.223 | 29.319 | 32.299 | 1.00 | 17.73 |
| ATOM | 798 | O | VAL | 898 | 26.093 | 30.052 | 32.698 | 1.00 | 18.12 |
| ATOM | 799 | N | VAL | 899 | 24.299 | 28.767 | 33.115 | 1.00 | 16.89 |
| ATOM | 800 | H | VAL | 899 | 23.667 | 28.142 | 32.694 | 1.00 | 25.00 |
| ATOM | 801 | CA | VAL | 899 | 24.207 | 28.938 | 34.570 | 1.00 | 19.83 |
| ATOM | 802 | CB | VAL | 899 | 23.310 | 27.826 | 35.243 | 1.00 | 18.01 |
| ATOM | 803 | CG1 | VAL | 899 | 23.912 | 26.398 | 34.998 | 1.00 | 18.61 |
| ATOM | 804 | CG2 | VAL | 899 | 23.143 | 28.200 | 36.771 | 1.00 | 14.95 |
| ATOM | 805 | C | VAL | 899 | 25.596 | 28.923 | 35.112 | 1.00 | 25.05 |
| ATOM | 806 | O | VAL | 899 | 26.373 | 27.996 | 34.865 | 1.00 | 30.47 |
| ATOM | 807 | N | ASN | 900 | 25.979 | 29.921 | 35.920 | 1.00 | 26.67 |
| ATOM | 808 | H | ASN | 900 | 25.322 | 30.564 | 36.239 | 1.00 | 25.00 |
| ATOM | 809 | CA | ASN | 900 | 27.385 | 30.058 | 36.379 | 1.00 | 24.86 |
| ATOM | 810 | CB | ASN | 900 | 27.735 | 31.705 | 36.533 | 1.00 | 35.53 |
| ATOM | 811 | CG | ASN | 900 | 27.575 | 32.456 | 35.171 | 1.00 | 43.56 |
| ATOM | 812 | OD1 | ASN | 900 | 28.421 | 32.219 | 34.324 | 1.00 | 51.26 |
| ATOM | 813 | ND2 | ASN | 900 | 26.707 | 33.394 | 34.846 | 1.00 | 43.70 |
| ATOM | 814 | HD21 | ASN | 900 | 26.001 | 33.817 | 35.411 | 1.00 | 25.00 |
| ATOM | 815 | HD22 | ASN | 900 | 26.846 | 33.605 | 33.902 | 1.00 | 25.00 |
| ATOM | 816 | C | ASN | 900 | 27.765 | 29.330 | 37.584 | 1.00 | 20.50 |
| ATOM | 817 | O | ASN | 900 | 26.985 | 29.373 | 38.532 | 1.00 | 19.39 |
| ATOM | 818 | N | LEU | 901 | 28.995 | 28.834 | 37.568 | 1.00 | 18.94 |
| ATOM | 819 | H | LEU | 901 | 29.498 | 28.938 | 36.726 | 1.00 | 25.00 |
| ATOM | 820 | CA | LEU | 901 | 29.674 | 28.113 | 38.705 | 1.00 | 19.97 |
| ATOM | 821 | CB | LEU | 901 | 30.765 | 27.149 | 38.078 | 1.00 | 15.79 |
| ATOM | 822 | CG | LEU | 901 | 31.901 | 26.471 | 38.869 | 1.00 | 15.10 |
| ATOM | 823 | CD1 | LEU | 901 | 31.290 | 25.510 | 39.870 | 1.00 | 16.96 |
| ATOM | 824 | CD2 | LEU | 901 | 32.874 | 25.770 | 37.933 | 1.00 | 13.24 |
| ATOM | 825 | C | LEU | 901 | 30.307 | 29.133 | 39.694 | 1.00 | 20.43 |
| ATOM | 826 | O | LEU | 901 | 31.171 | 29.937 | 39.325 | 1.00 | 23.64 |
| ATOM | 827 | N | LEU | 902 | 29.790 | 29.141 | 40.902 | 1.00 | 19.61 |
| ATOM | 828 | H | LEU | 902 | 29.062 | 28.499 | 41.004 | 1.00 | 25.00 |
| ATOM | 829 | CA | LEU | 902 | 30.167 | 29.906 | 42.085 | 1.00 | 19.28 |
| ATOM | 830 | CB | LEU | 902 | 29.039 | 30.039 | 42.963 | 1.00 | 16.95 |
| ATOM | 831 | CG | LEU | 902 | 28.058 | 31.002 | 42.303 | 1.00 | 15.93 |
| ATOM | 832 | CD1 | LEU | 902 | 27.179 | 31.595 | 43.352 | 1.00 | 19.17 |
| ATOM | 833 | CD2 | LEU | 902 | 28.768 | 32.241 | 41.744 | 1.00 | 19.91 |
| ATOM | 834 | C | LEU | 902 | 31.232 | 29.174 | 42.864 | 1.00 | 25.70 |
| ATOM | 835 | O | LEU | 902 | 32.292 | 29.690 | 43.252 | 1.00 | 27.86 |
| ATOM | 836 | N | GLY | 903 | 31.040 | 27.915 | 43.221 | 1.00 | 28.05 |
| ATOM | 837 | H | GLY | 903 | 30.231 | 27.444 | 43.019 | 1.00 | 25.00 |
| ATOM | 838 | CA | GLY | 903 | 32.162 | 27.187 | 43.913 | 1.00 | 28.51 |
| ATOM | 839 | C | GLY | 903 | 31.839 | 25.744 | 44.202 | 1.00 | 29.91 |
| ATOM | 840 | O | GLY | 903 | 30.900 | 25.223 | 43.589 | 1.00 | 32.39 |
| ATOM | 841 | N | ALA | 904 | 32.598 | 25.001 | 45.028 | 1.00 | 31.75 |
| ATOM | 842 | H | ALA | 904 | 33.451 | 25.355 | 45.342 | 1.00 | 25.00 |
| ATOM | 843 | CA | ALA | 904 | 32.202 | 23.592 | 45.416 | 1.00 | 30.05 |
| ATOM | 844 | CB | ALA | 904 | 32.726 | 22.482 | 44.514 | 1.00 | 29.37 |
| ATOM | 845 | C | ALA | 904 | 32.717 | 23.126 | 46.723 | 1.00 | 28.90 |
| ATOM | 846 | O | ALA | 904 | 33.824 | 23.462 | 47.145 | 1.00 | 30.19 |
| ATOM | 847 | N | CYS | 905 | 31.917 | 22.300 | 47.349 | 1.00 | 29.43 |
| ATOM | 848 | H | CYS | 905 | 30.979 | 22.187 | 47.049 | 1.00 | 25.00 |
| ATOM | 849 | CA | CYS | 905 | 32.312 | 21.627 | 48.585 | 1.00 | 27.11 |
| ATOM | 850 | CB | CYS | 905 | 31.154 | 21.501 | 49.480 | 1.00 | 27.19 |
| ATOM | 851 | SG | CYS | 905 | 30.577 | 23.110 | 49.892 | 1.00 | 26.51 |
| ATOM | 852 | C | CYS | 905 | 32.808 | 20.218 | 48.249 | 1.00 | 28.76 |
| ATOM | 853 | O | CYS | 905 | 32.045 | 19.272 | 48.150 | 1.00 | 28.03 |
| ATOM | 854 | N | THR | 906 | 34.053 | 20.129 | 47.855 | 1.00 | 31.77 |
| ATOM | 855 | H | THR | 906 | 34.584 | 20.963 | 47.768 | 1.00 | 25.00 |
| ATOM | 856 | CA | THR | 906 | 34.735 | 18.817 | 47.510 | 1.00 | 35.53 |
| ATOM | 857 | CB | THR | 906 | 35.731 | 19.177 | 46.403 | 1.00 | 33.82 |
| ATOM | 858 | OG1 | THR | 906 | 34.920 | 19.644 | 45.356 | 1.00 | 38.29 |
| ATOM | 859 | HG1 | THR | 906 | 35.484 | 19.904 | 44.625 | 1.00 | 25.00 |
| ATOM | 860 | CG2 | THR | 906 | 36.589 | 18.088 | 45.877 | 1.00 | 37.36 |
| ATOM | 861 | C | THR | 906 | 35.496 | 17.991 | 48.636 | 1.00 | 38.55 |
| ATOM | 862 | O | THR | 906 | 35.969 | 16.839 | 48.451 | 1.00 | 36.84 |
| ATOM | 863 | N | LYS | 907 | 35.674 | 18.632 | 49.832 | 1.00 | 40.74 |
| ATOM | 864 | H | LYS | 907 | 35.214 | 19.492 | 49.989 | 1.00 | 25.00 |
| ATOM | 865 | CA | LYS | 907 | 36.553 | 18.042 | 50.870 | 1.00 | 40.42 |
| ATOM | 866 | CB | LYS | 907 | 36.912 | 19.135 | 51.960 | 1.00 | 39.21 |
| ATOM | 867 | C | LYS | 907 | 36.052 | 16.791 | 51.515 | 1.00 | 42.10 |
| ATOM | 868 | O | LYS | 907 | 34.860 | 16.539 | 51.551 | 1.00 | 39.53 |
| ATOM | 869 | N | PRO | 908 | 37.002 | 15.956 | 51.993 | 1.00 | 41.69 |
| ATOM | 870 | CD | PRO | 908 | 38.467 | 16.086 | 51.820 | 1.00 | 39.65 |
| ATOM | 871 | CA | PRO | 908 | 36.646 | 14.589 | 52.282 | 1.00 | 38.81 |
| ATOM | 872 | CB | PRO | 908 | 38.055 | 14.084 | 52.511 | 1.00 | 40.86 |
| ATOM | 873 | CG | PRO | 908 | 38.778 | 14.719 | 51.350 | 1.00 | 38.24 |
| ATOM | 874 | C | PRO | 908 | 35.563 | 14.129 | 53.258 | 1.00 | 38.21 |
| ATOM | 875 | O | PRO | 908 | 34.777 | 13.203 | 52.944 | 1.00 | 38.86 |
| ATOM | 876 | N | GLY | 909 | 35.397 | 14.733 | 54.407 | 1.00 | 36.78 |
| ATOM | 877 | H | GLY | 909 | 35.852 | 15.553 | 54.682 | 1.00 | 25.00 |
| ATOM | 878 | CA | GLY | 909 | 34.322 | 14.113 | 55.242 | 1.00 | 36.67 |
| ATOM | 879 | C | GLY | 909 | 32.885 | 14.446 | 54.818 | 1.00 | 35.83 |
| ATOM | 880 | O | GLY | 909 | 31.939 | 14.694 | 55.599 | 1.00 | 36.42 |
| ATOM | 881 | N | GLY | 910 | 32.633 | 14.443 | 53.521 | 1.00 | 35.31 |
| ATOM | 882 | H | GLY | 910 | 33.210 | 14.048 | 52.829 | 1.00 | 25.00 |
| ATOM | 883 | CA | GLY | 910 | 31.266 | 14.797 | 53.125 | 1.00 | 30.42 |
| ATOM | 884 | C | GLY | 910 | 31.023 | 14.813 | 51.658 | 1.00 | 27.60 |
| ATOM | 885 | O | GLY | 910 | 31.889 | 14.568 | 50.792 | 1.00 | 28.33 |
| ATOM | 886 | N | PRO | 911 | 29.806 | 15.203 | 51.436 | 1.00 | 25.68 |
| ATOM | 887 | CD | PRO | 911 | 29.008 | 15.990 | 52.364 | 1.00 | 25.39 |
| ATOM | 888 | CA | PRO | 911 | 29.150 | 15.027 | 50.145 | 1.00 | 25.05 |
| ATOM | 889 | CB | PRO | 911 | 27.691 | 15.157 | 50.487 | 1.00 | 25.02 |
| ATOM | 890 | CG | PRO | 911 | 27.651 | 15.461 | 51.999 | 1.00 | 26.91 |
| ATOM | 891 | C | PRO | 911 | 29.561 | 15.994 | 49.051 | 1.00 | 24.74 |
| ATOM | 892 | O | PRO | 911 | 29.504 | 17.153 | 49.332 | 1.00 | 30.61 |
| ATOM | 893 | N | LEU | 912 | 29.949 | 15.732 | 47.804 | 1.00 | 23.23 |
| ATOM | 894 | H | LEU | 912 | 29.914 | 14.792 | 47.562 | 1.00 | 25.00 |
| ATOM | 895 | CA | LEU | 912 | 30.252 | 16.776 | 46.810 | 1.00 | 20.49 |
| ATOM | 896 | CB | LEU | 912 | 30.709 | 16.107 | 45.648 | 1.00 | 19.77 |
| ATOM | 897 | CG | LEU | 912 | 31.110 | 16.788 | 44.412 | 1.00 | 19.97 |
| ATOM | 898 | CD1 | LEU | 912 | 32.297 | 17.605 | 44.579 | 1.00 | 13.85 |
| ATOM | 899 | CD2 | LEU | 912 | 31.501 | 15.607 | 43.408 | 1.00 | 20.09 |
| ATOM | 900 | C | LEU | 912 | 29.001 | 17.568 | 46.473 | 1.00 | 22.67 |
| ATOM | 901 | O | LEU | 912 | 27.867 | 17.157 | 46.234 | 1.00 | 23.53 |
| ATOM | 902 | N | MET | 913 | 29.271 | 18.856 | 46.470 | 1.00 | 22.84 |
| ATOM | 903 | H | MET | 913 | 30.140 | 19.069 | 46.865 | 1.00 | 25.00 |
| ATOM | 904 | CA | MET | 913 | 28.352 | 19.950 | 46.237 | 1.00 | 17.94 |
| ATOM | 905 | CB | MET | 913 | 28.232 | 20.520 | 47.585 | 1.00 | 14.19 |
| ATOM | 906 | CG | MET | 913 | 27.284 | 19.601 | 48.299 | 1.00 | 12.86 |
| ATOM | 907 | SD | MET | 913 | 27.325 | 20.499 | 49.776 | 1.00 | 23.04 |
| ATOM | 908 | CE | MET | 913 | 25.729 | 20.269 | 50.362 | 1.00 | 16.35 |
| ATOM | 909 | C | MET | 913 | 28.934 | 20.881 | 45.168 | 1.00 | 20.06 |
| ATOM | 910 | O | MET | 913 | 30.044 | 21.385 | 45.276 | 1.00 | 20.36 |
| ATOM | 911 | N | VAL | 914 | 28.211 | 21.170 | 44.070 | 1.00 | 21.14 |
| ATOM | 912 | H | VAL | 914 | 27.320 | 20.794 | 44.055 | 1.00 | 25.00 |
| ATOM | 913 | CA | VAL | 914 | 28.604 | 22.059 | 42.876 | 1.00 | 18.64 |
| ATOM | 914 | CB | VAL | 914 | 28.380 | 21.229 | 41.524 | 1.00 | 20.20 |
| ATOM | 915 | CG1 | VAL | 914 | 28.894 | 21.897 | 40.270 | 1.00 | 19.79 |
| ATOM | 916 | CG2 | VAL | 914 | 29.172 | 19.920 | 41.674 | 1.00 | 16.97 |
| ATOM | 917 | C | VAL | 914 | 27.614 | 23.235 | 43.016 | 1.00 | 16.15 |
| ATOM | 918 | O | VAL | 914 | 26.384 | 23.100 | 43.013 | 1.00 | 9.92 |
| ATOM | 919 | N | ILE | 915 | 28.193 | 24.341 | 43.535 | 1.00 | 18.42 |
| ATOM | 920 | H | ILE | 915 | 29.182 | 24.367 | 43.576 | 1.00 | 25.00 |
| ATOM | 921 | CA | ILE | 915 | 27.392 | 25.573 | 43.832 | 1.00 | 18.11 |
| ATOM | 922 | CB | ILE | 915 | 27.980 | 26.206 | 45.174 | 1.00 | 16.93 |
| ATOM | 923 | CG2 | ILE | 915 | 27.077 | 27.396 | 45.585 | 1.00 | 14.95 |
| ATOM | 924 | CG1 | ILE | 915 | 27.851 | 25.252 | 46.430 | 1.00 | 14.66 |
| ATOM | 925 | CD1 | ILE | 915 | 29.176 | 25.426 | 47.165 | 1.00 | 9.89 |
| ATOM | 926 | C | ILE | 915 | 27.345 | 26.563 | 42.635 | 1.00 | 18.08 |
| ATOM | 927 | O | ILE | 915 | 28.380 | 27.000 | 42.111 | 1.00 | 18.40 |
| ATOM | 928 | N | VAL | 916 | 26.126 | 26.738 | 42.073 | 1.00 | 16.69 |
| ATOM | 929 | H | VAL | 916 | 25.344 | 26.304 | 42.468 | 1.00 | 25.00 |
| ATOM | 930 | CA | VAL | 916 | 25.968 | 27.611 | 40.947 | 1.00 | 14.59 |
| ATOM | 931 | CB | VAL | 916 | 25.360 | 26.831 | 39.639 | 1.00 | 16.42 |
| ATOM | 932 | CG1 | VAL | 916 | 26.429 | 25.915 | 38.858 | 1.00 | 14.26 |
| ATOM | 933 | CG2 | VAL | 916 | 24.083 | 26.163 | 40.112 | 1.00 | 14.36 |
| ATOM | 934 | C | VAL | 916 | 25.050 | 28.735 | 41.353 | 1.00 | 12.51 |
| ATOM | 935 | O | VAL | 916 | 24.337 | 28.622 | 42.379 | 1.00 | 9.55 |
| ATOM | 936 | N | GLU | 917 | 25.170 | 29.867 | 40.622 | 1.00 | 13.92 |
| ATOM | 937 | H | GLU | 917 | 25.810 | 29.911 | 39.863 | 1.00 | 25.00 |
| ATOM | 938 | CA | GLU | 917 | 24.194 | 31.002 | 40.932 | 1.00 | 17.96 |
| ATOM | 939 | CB | GLU | 917 | 24.346 | 32.008 | 39.831 | 1.00 | 20.40 |
| ATOM | 940 | CG | GLU | 917 | 23.729 | 31.442 | 38.546 | 1.00 | 20.95 |
| ATOM | 941 | CD | GLU | 917 | 23.745 | 32.607 | 37.651 | 1.00 | 23.60 |
| ATOM | 942 | OE1 | GLU | 917 | 24.612 | 32.734 | 36.805 | 1.00 | 25.18 |
| ATOM | 943 | OE2 | GLU | 917 | 22.937 | 33.475 | 37.829 | 1.00 | 23.69 |
| ATOM | 944 | C | GLU | 917 | 22.673 | 30.619 | 41.008 | 1.00 | 19.23 |
| ATOM | 945 | O | GLU | 917 | 22.231 | 29.704 | 40.330 | 1.00 | 22.71 |
| ATOM | 946 | N | PHE | 918 | 21.812 | 31.217 | 41.814 | 1.00 | 19.61 |
| ATOM | 947 | H | PHE | 918 | 22.174 | 31.906 | 42.412 | 1.00 | 25.00 |
| ATOM | 948 | CA | PHE | 918 | 20.427 | 30.826 | 41.891 | 1.00 | 15.46 |
| ATOM | 949 | CB | PHE | 918 | 19.968 | 31.005 | 43.307 | 1.00 | 15.60 |
| ATOM | 950 | CG | PHE | 918 | 18.544 | 30.809 | 43.621 | 1.00 | 16.12 |
| ATOM | 951 | CD1 | PHE | 918 | 18.007 | 29.588 | 43.754 | 1.00 | 15.46 |
| ATOM | 952 | CD2 | PHE | 918 | 17.716 | 31.887 | 43.892 | 1.00 | 18.48 |
| ATOM | 953 | CE1 | PHE | 918 | 16.667 | 29.471 | 44.153 | 1.00 | 15.56 |
| ATOM | 954 | CE2 | PHE | 918 | 16.388 | 31.810 | 44.294 | 1.00 | 13.68 |
| ATOM | 955 | CZ | PHE | 918 | 15.862 | 30.552 | 44.423 | 1.00 | 15.51 |
| ATOM | 956 | C | PHE | 918 | 19.584 | 31.674 | 40.958 | 1.00 | 20.84 |
| ATOM | 957 | O | PHE | 918 | 19.574 | 32.887 | 41.015 | 1.00 | 22.86 |
| ATOM | 958 | N | CYS | 919 | 18.701 | 31.002 | 40.146 | 1.00 | 24.67 |
| ATOM | 959 | H | CYS | 919 | 18.773 | 30.057 | 40.350 | 1.00 | 25.00 |
| ATOM | 960 | CA | CYS | 919 | 17.704 | 31.395 | 39.104 | 1.00 | 18.54 |
| ATOM | 961 | CB | CYS | 919 | 17.856 | 30.408 | 37.965 | 1.00 | 8.24 |
| ATOM | 962 | SG | CYS | 919 | 19.415 | 30.597 | 37.184 | 1.00 | 17.14 |
| ATOM | 963 | C | CYS | 919 | 16.288 | 31.397 | 39.713 | 1.00 | 20.79 |
| ATOM | 964 | O | CYS | 919 | 15.504 | 30.427 | 39.723 | 1.00 | 19.09 |
| ATOM | 965 | N | LYS | 920 | 15.982 | 32.659 | 40.157 | 1.00 | 24.06 |
| ATOM | 966 | H | LYS | 920 | 16.664 | 33.360 | 40.093 | 1.00 | 25.00 |
| ATOM | 967 | CA | LYS | 920 | 14.714 | 32.965 | 40.877 | 1.00 | 28.12 |
| ATOM | 968 | CB | LYS | 920 | 14.376 | 34.469 | 41.038 | 1.00 | 31.72 |
| ATOM | 969 | CG | LYS | 920 | 14.673 | 35.092 | 42.365 | 1.00 | 41.66 |
| ATOM | 970 | CD | LYS | 920 | 16.275 | 35.120 | 42.604 | 1.00 | 50.37 |
| ATOM | 971 | CE | LYS | 920 | 16.993 | 35.837 | 43.845 | 1.00 | 52.49 |
| ATOM | 972 | NZ | LYS | 920 | 18.462 | 35.494 | 43.887 | 1.00 | 57.05 |
| ATOM | 973 | HZ1 | LYS | 920 | 18.878 | 35.618 | 42.939 | 1.00 | 25.00 |
| ATOM | 974 | HZ2 | LYS | 920 | 18.630 | 34.513 | 44.191 | 1.00 | 25.00 |
| ATOM | 975 | HZ3 | LYS | 920 | 18.958 | 36.121 | 44.546 | 1.00 | 25.00 |
| ATOM | 976 | C | LYS | 920 | 13.472 | 32.494 | 40.217 | 1.00 | 29.65 |
| ATOM | 977 | O | LYS | 920 | 12.534 | 32.181 | 40.963 | 1.00 | 29.39 |
| ATOM | 978 | N | PHE | 921 | 13.371 | 32.678 | 38.844 | 1.00 | 28.19 |
| ATOM | 979 | H | PHE | 921 | 14.135 | 32.923 | 38.268 | 1.00 | 25.00 |
| ATOM | 980 | CA | PHE | 921 | 12.099 | 32.285 | 38.148 | 1.00 | 30.76 |
| ATOM | 981 | CB | PHE | 921 | 11.881 | 33.227 | 36.847 | 1.00 | 32.02 |
| ATOM | 982 | CG | PHE | 921 | 11.873 | 34.553 | 37.452 | 1.00 | 35.37 |
| ATOM | 983 | CD1 | PHE | 921 | 10.854 | 34.950 | 38.383 | 1.00 | 37.21 |
| ATOM | 984 | CD2 | PHE | 921 | 12.937 | 35.449 | 37.136 | 1.00 | 36.75 |
| ATOM | 985 | CE1 | PHE | 921 | 11.017 | 36.183 | 39.098 | 1.00 | 37.21 |
| ATOM | 986 | CE2 | PHE | 921 | 13.100 | 36.669 | 37.834 | 1.00 | 35.67 |
| ATOM | 987 | CZ | PHE | 921 | 12.109 | 37.046 | 38.774 | 1.00 | 33.19 |
| ATOM | 988 | C | PHE | 921 | 11.855 | 30.798 | 37.730 | 1.00 | 29.64 |
| ATOM | 989 | O | PHE | 921 | 10.858 | 30.436 | 37.099 | 1.00 | 30.71 |
| ATOM | 990 | N | GLY | 922 | 12.755 | 29.940 | 38.132 | 1.00 | 26.21 |
| ATOM | 991 | H | GLY | 922 | 13.501 | 30.300 | 38.675 | 1.00 | 25.00 |
| ATOM | 992 | CA | GLY | 922 | 12.606 | 28.561 | 37.785 | 1.00 | 23.78 |
| ATOM | 993 | C | GLY | 922 | 12.996 | 28.369 | 36.365 | 1.00 | 23.63 |
| ATOM | 994 | O | GLY | 922 | 13.779 | 29.148 | 35.854 | 1.00 | 22.46 |
| ATOM | 995 | N | ASN | 923 | 12.636 | 27.228 | 35.763 | 1.00 | 26.68 |
| ATOM | 996 | H | ASN | 923 | 12.158 | 26.631 | 36.357 | 1.00 | 25.00 |
| ATOM | 997 | CA | ASN | 923 | 12.912 | 26.876 | 34.352 | 1.00 | 25.76 |
| ATOM | 998 | CB | ASN | 923 | 12.975 | 25.307 | 34.262 | 1.00 | 25.38 |
| ATOM | 999 | CG | ASN | 923 | 11.750 | 24.614 | 34.814 | 1.00 | 26.84 |
| ATOM | 1000 | OD1 | ASN | 923 | 10.631 | 24.644 | 34.364 | 1.00 | 33.94 |
| ATOM | 1001 | ND2 | ASN | 923 | 11.616 | 24.155 | 35.987 | 1.00 | 32.02 |
| ATOM | 1002 | HD21 | ASN | 923 | 12.284 | 24.222 | 36.693 | 1.00 | 25.00 |
| ATOM | 1003 | HD22 | ASN | 923 | 10.771 | 23.732 | 36.191 | 1.00 | 25.00 |
| ATOM | 1004 | C | ASN | 923 | 11.845 | 27.513 | 33.447 | 1.00 | 26.34 |
| ATOM | 1005 | O | ASN | 923 | 10.695 | 27.804 | 33.776 | 1.00 | 21.54 |
| ATOM | 1006 | N | LEU | 924 | 12.220 | 27.779 | 32.261 | 1.00 | 26.55 |
| ATOM | 1007 | H | LEU | 924 | 13.101 | 27.554 | 31.951 | 1.00 | 25.00 |
| ATOM | 1008 | CA | LEU | 924 | 11.363 | 28.424 | 31.344 | 1.00 | 29.33 |
| ATOM | 1009 | CB | LEU | 924 | 12.153 | 28.587 | 30.063 | 1.00 | 29.04 |
| ATOM | 1010 | CG | LEU | 924 | 12.280 | 29.909 | 29.333 | 1.00 | 27.24 |
| ATOM | 1011 | CD1 | LEU | 924 | 12.564 | 31.066 | 30.231 | 1.00 | 25.66 |
| ATOM | 1012 | CD2 | LEU | 924 | 13.355 | 29.692 | 28.333 | 1.00 | 26.87 |
| ATOM | 1013 | C | LEU | 924 | 10.096 | 27.646 | 31.126 | 1.00 | 32.23 |
| ATOM | 1014 | O | LEU | 924 | 9.022 | 28.200 | 31.298 | 1.00 | 37.86 |
| ATOM | 1015 | N | SER | 925 | 10.071 | 26.408 | 30.743 | 1.00 | 32.25 |
| ATOM | 1016 | H | SER | 925 | 10.959 | 25.987 | 30.639 | 1.00 | 25.00 |
| ATOM | 1017 | CA | SER | 925 | 8.816 | 25.667 | 30.515 | 1.00 | 28.04 |
| ATOM | 1018 | CB | SER | 925 | 9.143 | 24.200 | 30.416 | 1.00 | 31.83 |
| ATOM | 1019 | OG | SER | 925 | 9.887 | 23.664 | 31.559 | 1.00 | 27.82 |
| ATOM | 1020 | HG | SER | 925 | 9.933 | 22.717 | 31.575 | 1.00 | 25.00 |
| ATOM | 1021 | C | SER | 925 | 7.772 | 25.863 | 31.571 | 1.00 | 29.30 |
| ATOM | 1022 | O | SER | 925 | 6.622 | 26.006 | 31.174 | 1.00 | 31.02 |
| ATOM | 1023 | N | THR | 926 | 8.043 | 25.860 | 32.907 | 1.00 | 28.53 |
| ATOM | 1024 | H | THR | 926 | 8.957 | 25.681 | 33.252 | 1.00 | 25.00 |
| ATOM | 1025 | CA | THR | 926 | 6.966 | 26.129 | 33.922 | 1.00 | 27.45 |
| ATOM | 1026 | CB | THR | 926 | 7.461 | 25.780 | 35.355 | 1.00 | 28.37 |
| ATOM | 1027 | OG1 | THR | 926 | 7.998 | 24.502 | 35.282 | 1.00 | 30.93 |
| ATOM | 1028 | HG1 | THR | 926 | 8.301 | 24.282 | 36.167 | 1.00 | 25.00 |
| ATOM | 1029 | CG2 | THR | 926 | 6.383 | 25.482 | 36.401 | 1.00 | 27.02 |
| ATOM | 1030 | C | THR | 926 | 6.525 | 27.582 | 33.900 | 1.00 | 26.26 |
| ATOM | 1031 | O | THR | 926 | 5.374 | 27.950 | 33.992 | 1.00 | 28.17 |
| ATOM | 1032 | N | TYR | 927 | 7.488 | 28.442 | 33.756 | 1.00 | 27.50 |
| ATOM | 1033 | H | TYR | 927 | 8.384 | 28.038 | 33.660 | 1.00 | 25.00 |
| ATOM | 1034 | CA | TYR | 927 | 7.345 | 29.919 | 33.694 | 1.00 | 27.56 |
| ATOM | 1035 | CB | TYR | 927 | 8.748 | 30.667 | 33.565 | 1.00 | 30.80 |
| ATOM | 1036 | CG | TYR | 927 | 8.499 | 32.103 | 33.572 | 1.00 | 31.81 |
| ATOM | 1037 | CD1 | TYR | 927 | 7.844 | 32.562 | 34.702 | 1.00 | 33.71 |
| ATOM | 1038 | CE1 | TYR | 927 | 7.601 | 33.934 | 34.881 | 1.00 | 35.75 |
| ATOM | 1039 | CD2 | TYR | 927 | 8.914 | 33.002 | 32.567 | 1.00 | 32.25 |
| ATOM | 1040 | CE2 | TYR | 927 | 8.674 | 34.388 | 32.735 | 1.00 | 35.54 |
| ATOM | 1041 | CZ | TYR | 927 | 8.012 | 34.853 | 33.909 | 1.00 | 36.98 |
| ATOM | 1042 | OH | TYR | 927 | 7.811 | 36.188 | 34.219 | 1.00 | 36.57 |
| ATOM | 1043 | HH | TYR | 927 | 8.165 | 36.735 | 33.511 | 1.00 | 25.00 |
| ATOM | 1044 | C | TYR | 927 | 6.523 | 30.316 | 32.519 | 1.00 | 25.01 |
| ATOM | 1045 | O | TYR | 927 | 5.503 | 30.923 | 32.781 | 1.00 | 25.33 |
| ATOM | 1046 | N | LEU | 928 | 6.944 | 30.086 | 31.289 | 1.00 | 21.20 |
| ATOM | 1047 | H | LEU | 928 | 7.876 | 29.814 | 31.144 | 1.00 | 25.00 |
| ATOM | 1048 | CA | LEU | 928 | 6.077 | 30.462 | 30.211 | 1.00 | 22.72 |
| ATOM | 1049 | CB | LEU | 928 | 6.661 | 30.032 | 28.936 | 1.00 | 22.10 |
| ATOM | 1050 | CG | LEU | 928 | 7.869 | 30.885 | 28.543 | 1.00 | 23.43 |
| ATOM | 1051 | CD1 | LEU | 928 | 8.640 | 30.200 | 27.427 | 1.00 | 24.59 |
| ATOM | 1052 | CD2 | LEU | 928 | 7.455 | 32.161 | 27.825 | 1.00 | 26.33 |
| ATOM | 1053 | C | LEU | 928 | 4.719 | 29.863 | 30.350 | 1.00 | 26.13 |
| ATOM | 1054 | O | LEU | 928 | 3.690 | 30.485 | 30.081 | 1.00 | 29.10 |
| ATOM | 1055 | N | ARG | 929 | 4.547 | 28.685 | 30.917 | 1.00 | 31.09 |
| ATOM | 1056 | H | ARG | 929 | 5.262 | 28.171 | 31.330 | 1.00 | 25.00 |
| ATOM | 1057 | CA | ARG | 929 | 3.133 | 28.140 | 30.993 | 1.00 | 33.63 |
| ATOM | 1058 | CB | ARG | 929 | 3.159 | 26.586 | 31.275 | 1.00 | 35.07 |
| ATOM | 1059 | CG | ARG | 929 | 2.167 | 25.451 | 30.789 | 1.00 | 39.72 |
| ATOM | 1060 | CD | ARG | 929 | 3.104 | 24.080 | 30.814 | 1.00 | 47.07 |
| ATOM | 1061 | NE | ARG | 929 | 3.829 | 23.605 | 32.092 | 1.00 | 49.01 |
| ATOM | 1062 | HE | ARG | 929 | 3.301 | 23.682 | 32.918 | 1.00 | 25.00 |
| ATOM | 1063 | CZ | ARG | 929 | 5.118 | 23.145 | 32.187 | 1.00 | 45.25 |
| ATOM | 1064 | NH1 | ARG | 929 | 5.652 | 22.746 | 33.373 | 1.00 | 40.92 |
| ATOM | 1065 | HH11 | ARG | 929 | 5.077 | 22.813 | 34.191 | 1.00 | 25.00 |
| ATOM | 1066 | HH12 | ARG | 929 | 6.604 | 22.465 | 33.473 | 1.00 | 25.00 |
| ATOM | 1067 | NH2 | ARG | 929 | 5.845 | 23.191 | 31.090 | 1.00 | 45.30 |
| ATOM | 1068 | HH21 | ARG | 929 | 5.442 | 23.523 | 30.238 | 1.00 | 25.00 |
| ATOM | 1069 | HH22 | ARG | 929 | 6.798 | 22.898 | 31.129 | 1.00 | 25.00 |
| ATOM | 1070 | C | ARG | 929 | 2.287 | 28.846 | 32.027 | 1.00 | 33.85 |
| ATOM | 1071 | O | ARG | 929 | 1.041 | 28.736 | 32.149 | 1.00 | 35.17 |
| ATOM | 1072 | N | SER | 930 | 2.966 | 29.578 | 32.898 | 1.00 | 35.91 |
| ATOM | 1073 | H | SER | 930 | 3.937 | 29.716 | 32.773 | 1.00 | 25.00 |
| ATOM | 1074 | CA | SER | 930 | 2.199 | 30.348 | 33.990 | 1.00 | 37.28 |
| ATOM | 1075 | CB | SER | 930 | 2.961 | 30.469 | 35.328 | 1.00 | 34.96 |
| ATOM | 1076 | OG | SER | 930 | 4.214 | 31.161 | 35.065 | 1.00 | 32.16 |
| ATOM | 1077 | HG | SER | 930 | 4.670 | 31.323 | 35.896 | 1.00 | 25.00 |
| ATOM | 1078 | C | SER | 930 | 1.842 | 31.818 | 33.596 | 1.00 | 38.36 |
| ATOM | 1079 | O | SER | 930 | 0.960 | 32.459 | 34.150 | 1.00 | 38.66 |
| ATOM | 1080 | N | LYS | 931 | 2.509 | 32.370 | 32.570 | 1.00 | 38.87 |
| ATOM | 1081 | H | LYS | 931 | 3.168 | 31.787 | 32.128 | 1.00 | 25.00 |
| ATOM | 1082 | CA | LYS | 931 | 2.346 | 33.714 | 32.021 | 1.00 | 39.53 |
| ATOM | 1083 | CB | LYS | 931 | 3.727 | 34.094 | 31.591 | 1.00 | 40.92 |
| ATOM | 1084 | CG | LYS | 931 | 4.694 | 34.221 | 32.752 | 1.00 | 39.55 |
| ATOM | 1085 | CD | LYS | 931 | 4.075 | 35.224 | 33.780 | 1.00 | 41.00 |
| ATOM | 1086 | CE | LYS | 931 | 4.310 | 36.709 | 33.330 | 1.00 | 39.36 |
| ATOM | 1087 | NZ | LYS | 931 | 4.808 | 36.765 | 31.936 | 1.00 | 39.26 |
| ATOM | 1088 | HZ1 | LYS | 931 | 4.181 | 36.207 | 31.333 | 1.00 | 25.00 |
| ATOM | 1089 | HZ2 | LYS | 931 | 5.790 | 36.426 | 31.866 | 1.00 | 25.00 |
| ATOM | 1090 | HZ3 | LYS | 931 | 4.759 | 37.749 | 31.596 | 1.00 | 25.00 |
| ATOM | 1091 | C | LYS | 931 | 1.361 | 33.774 | 30.858 | 1.00 | 41.94 |
| ATOM | 1092 | O | LYS | 931 | 1.283 | 34.720 | 30.063 | 1.00 | 43.49 |
| ATOM | 1093 | N | ARG | 932 | 0.562 | 32.727 | 30.784 | 1.00 | 42.67 |
| ATOM | 1094 | H | ARG | 932 | 0.651 | 32.065 | 31.505 | 1.00 | 25.00 |
| ATOM | 1095 | CA | ARG | 932 | −0.423 | 32.548 | 29.734 | 1.00 | 40.67 |
| ATOM | 1096 | CB | ARG | 932 | −1.157 | 31.214 | 29.991 | 1.00 | 35.92 |
| ATOM | 1097 | CG | ARG | 932 | −0.396 | 30.026 | 29.360 | 1.00 | 32.12 |
| ATOM | 1098 | CD | ARG | 932 | −0.351 | 29.896 | 27.871 | 1.00 | 27.19 |
| ATOM | 1099 | NE | ARG | 932 | 0.502 | 28.740 | 27.438 | 1.00 | 26.92 |
| ATOM | 1100 | HE | ARG | 932 | 1.455 | 28.907 | 27.274 | 1.00 | 25.00 |
| ATOM | 1101 | CZ | ARG | 932 | 0.047 | 27.524 | 27.168 | 1.00 | 24.63 |
| ATOM | 1102 | NH1 | ARG | 932 | 0.833 | 26.584 | 26.733 | 1.00 | 22.28 |
| ATOM | 1103 | HH11 | ARG | 932 | 1.808 | 26.759 | 26.571 | 1.00 | 25.00 |
| ATOM | 1104 | HH12 | ARG | 932 | 0.447 | 25.675 | 26.560 | 1.00 | 25.00 |
| ATOM | 1105 | NH2 | ARG | 932 | −1.212 | 27.237 | 27.298 | 1.00 | 24.07 |
| ATOM | 1106 | HH21 | ARG | 932 | −1.830 | 27.954 | 27.613 | 1.00 | 25.00 |
| ATOM | 1107 | HH22 | ARG | 932 | −1.556 | 26.317 | 27.126 | 1.00 | 25.00 |
| ATOM | 1108 | C | ARG | 932 | −1.346 | 33.725 | 29.795 | 1.00 | 43.62 |
| ATOM | 1109 | O | ARG | 932 | −1.593 | 34.483 | 28.850 | 1.00 | 45.89 |
| ATOM | 1110 | N | ASN | 933 | −1.835 | 33.906 | 30.993 | 1.00 | 45.70 |
| ATOM | 1111 | H | ASN | 933 | −1.602 | 33.269 | 31.704 | 1.00 | 25.00 |
| ATOM | 1112 | CA | ASN | 933 | −2.791 | 35.004 | 31.242 | 1.00 | 45.46 |
| ATOM | 1113 | CB | ASN | 933 | −3.332 | 34.684 | 32.587 | 1.00 | 46.72 |
| ATOM | 1114 | CG | ASN | 933 | −4.604 | 35.381 | 32.756 | 1.00 | 46.41 |
| ATOM | 1115 | OD1 | ASN | 933 | −5.226 | 35.819 | 31.804 | 1.00 | 49.99 |
| ATOM | 1116 | ND2 | ASN | 933 | −5.067 | 35.450 | 33.957 | 1.00 | 47.69 |
| ATOM | 1117 | HD21 | ASN | 933 | −4.589 | 35.046 | 34.700 | 1.00 | 25.00 |
| ATOM | 1118 | HD22 | ASN | 933 | −5.920 | 35.907 | 34.086 | 1.00 | 25.00 |
| ATOM | 1119 | C | ASN | 933 | −2.154 | 36.379 | 31.224 | 1.00 | 45.37 |
| ATOM | 1120 | O | ASN | 933 | −2.813 | 37.390 | 31.423 | 1.00 | 48.59 |
| ATOM | 1121 | N | GLU | 934 | −0.820 | 36.422 | 31.082 | 1.00 | 42.02 |
| ATOM | 1122 | H | GLU | 934 | −0.347 | 35.617 | 30.766 | 1.00 | 25.00 |
| ATOM | 1123 | CA | GLU | 934 | −0.069 | 37.656 | 31.150 | 1.00 | 37.06 |
| ATOM | 1124 | CB | GLU | 934 | 0.633 | 37.690 | 32.587 | 1.00 | 36.65 |
| ATOM | 1125 | CG | GLU | 934 | −0.178 | 37.046 | 33.774 | 1.00 | 38.96 |
| ATOM | 1126 | CD | GLU | 934 | 0.444 | 37.051 | 35.209 | 1.00 | 41.32 |
| ATOM | 1127 | OE1 | GLU | 934 | −0.352 | 37.252 | 36.139 | 1.00 | 42.69 |
| ATOM | 1128 | OE2 | GLU | 934 | 1.660 | 36.846 | 35.413 | 1.00 | 43.89 |
| ATOM | 1129 | C | GLU | 934 | 0.924 | 37.784 | 30.023 | 1.00 | 34.23 |
| ATOM | 1130 | O | GLU | 934 | 2.108 | 38.032 | 30.263 | 1.00 | 37.60 |
| ATOM | 1131 | N | PHE | 935 | 0.440 | 37.796 | 28.831 | 1.00 | 28.95 |
| ATOM | 1132 | H | PHE | 935 | −0.526 | 37.811 | 28.758 | 1.00 | 25.00 |
| ATOM | 1133 | CA | PHE | 935 | 1.211 | 37.935 | 27.588 | 1.00 | 27.28 |
| ATOM | 1134 | CB | PHE | 935 | 1.433 | 36.639 | 26.806 | 1.00 | 23.84 |
| ATOM | 1135 | CG | PHE | 935 | 2.154 | 36.719 | 25.515 | 1.00 | 15.84 |
| ATOM | 1136 | CD1 | PHE | 935 | 1.522 | 36.704 | 24.334 | 1.00 | 17.66 |
| ATOM | 1137 | CD2 | PHE | 935 | 3.498 | 36.701 | 25.517 | 1.00 | 16.28 |
| ATOM | 1138 | CE1 | PHE | 935 | 2.238 | 36.653 | 23.116 | 1.00 | 19.26 |
| ATOM | 1139 | CE2 | PHE | 935 | 4.237 | 36.650 | 24.350 | 1.00 | 19.65 |
| ATOM | 1140 | CZ | PHE | 935 | 3.610 | 36.620 | 23.128 | 1.00 | 18.42 |
| ATOM | 1141 | C | PHE | 935 | 0.436 | 38.781 | 26.579 | 1.00 | 27.65 |
| ATOM | 1142 | O | PHE | 935 | −0.797 | 38.766 | 26.503 | 1.00 | 26.46 |
| ATOM | 1143 | N | VAL | 936 | 1.175 | 39.561 | 25.825 | 1.00 | 28.19 |
| ATOM | 1144 | H | VAL | 936 | 2.151 | 39.584 | 25.953 | 1.00 | 25.00 |
| ATOM | 1145 | CA | VAL | 936 | 0.599 | 40.382 | 24.778 | 1.00 | 30.16 |
| ATOM | 1146 | CB | VAL | 936 | 0.222 | 41.716 | 25.271 | 1.00 | 32.30 |
| ATOM | 1147 | CG1 | VAL | 936 | −1.227 | 41.494 | 25.799 | 1.00 | 31.05 |
| ATOM | 1148 | CG2 | VAL | 936 | 1.398 | 42.380 | 26.171 | 1.00 | 32.81 |
| ATOM | 1149 | C | VAL | 936 | 1.708 | 40.575 | 23.815 | 1.00 | 29.97 |
| ATOM | 1150 | O | VAL | 936 | 2.887 | 40.592 | 24.199 | 1.00 | 32.17 |
| ATOM | 1151 | N | PRO | 937 | 1.400 | 40.567 | 22.562 | 1.00 | 30.90 |
| ATOM | 1152 | CD | PRO | 937 | 0.132 | 40.158 | 21.940 | 1.00 | 29.99 |
| ATOM | 1153 | CA | PRO | 937 | 2.528 | 40.669 | 21.673 | 1.00 | 33.41 |
| ATOM | 1154 | CB | PRO | 937 | 1.971 | 40.228 | 20.304 | 1.00 | 34.43 |
| ATOM | 1155 | CG | PRO | 937 | 0.690 | 39.520 | 20.648 | 1.00 | 29.52 |
| ATOM | 1156 | C | PRO | 937 | 3.152 | 42.034 | 21.670 | 1.00 | 37.27 |
| ATOM | 1157 | O | PRO | 937 | 4.282 | 42.164 | 21.224 | 1.00 | 37.70 |
| ATOM | 1158 | N | TYR | 938 | 2.295 | 43.001 | 21.970 | 1.00 | 40.37 |
| ATOM | 1159 | H | TYR | 938 | 1.390 | 42.730 | 22.215 | 1.00 | 25.00 |
| ATOM | 1160 | CA | TYR | 938 | 2.568 | 44.436 | 22.015 | 1.00 | 50.77 |
| ATOM | 1161 | CB | TYR | 938 | 1.184 | 45.112 | 22.009 | 1.00 | 46.09 |
| ATOM | 1162 | C | TYR | 938 | 3.439 | 44.824 | 23.212 | 1.00 | 62.66 |
| ATOM | 1163 | O | TYR | 938 | 2.914 | 45.214 | 24.272 | 1.00 | 65.20 |
| ATOM | 1165 | CB | PHE | 999 | 3.911 | 40.787 | 32.454 | 1.00 | 50.06 |
| ATOM | 1166 | CG | PHE | 999 | 4.046 | 41.729 | 33.563 | 1.00 | 56.91 |
| ATOM | 1167 | CD1 | PHE | 999 | 3.582 | 41.330 | 34.811 | 1.00 | 59.28 |
| ATOM | 1168 | CD2 | PHE | 999 | 4.485 | 43.046 | 33.391 | 1.00 | 59.39 |
| ATOM | 1169 | CE1 | PHE | 999 | 3.534 | 42.222 | 35.878 | 1.00 | 59.75 |
| ATOM | 1170 | CE2 | PHE | 999 | 4.434 | 43.942 | 34.450 | 1.00 | 61.40 |
| ATOM | 1171 | CZ | PHE | 999 | 3.950 | 43.522 | 35.681 | 1.00 | 59.81 |
| ATOM | 1172 | C | PHE | 999 | 4.617 | 41.020 | 30.226 | 1.00 | 44.80 |
| ATOM | 1173 | O | PHE | 999 | 5.624 | 41.688 | 29.880 | 1.00 | 46.35 |
| ATOM | 1174 | HT1 | PHE | 999 | 1.816 | 40.773 | 30.310 | 1.00 | 25.00 |
| ATOM | 1175 | HT2 | PHE | 999 | 1.997 | 42.448 | 30.098 | 1.00 | 25.00 |
| ATOM | 1176 | N | PHE | 999 | 2.118 | 41.655 | 30.772 | 1.00 | 46.13 |
| ATOM | 1177 | HT3 | PHE | 999 | 1.537 | 41.873 | 31.612 | 1.00 | 25.00 |
| ATOM | 1178 | CA | PHE | 999 | 3.582 | 41.587 | 31.169 | 1.00 | 46.04 |
| ATOM | 1179 | N | LEU | 1000 | 4.401 | 39.728 | 29.883 | 1.00 | 40.30 |
| ATOM | 1180 | H | LEU | 1000 | 3.573 | 39.258 | 30.089 | 1.00 | 25.00 |
| ATOM | 1181 | CA | LEU | 1000 | 5.337 | 39.093 | 28.955 | 1.00 | 33.94 |
| ATOM | 1182 | CB | LEU | 1000 | 5.165 | 37.627 | 29.102 | 1.00 | 38.42 |
| ATOM | 1183 | CG | LEU | 1000 | 6.350 | 36.647 | 28.887 | 1.00 | 40.19 |
| ATOM | 1184 | CD1 | LEU | 1000 | 7.575 | 37.081 | 29.693 | 1.00 | 37.92 |
| ATOM | 1185 | CD2 | LEU | 1000 | 5.909 | 35.244 | 29.326 | 1.00 | 39.38 |
| ATOM | 1186 | C | LEU | 1000 | 4.983 | 39.560 | 27.625 | 1.00 | 28.82 |
| ATOM | 1187 | O | LEU | 1000 | 3.863 | 39.971 | 27.350 | 1.00 | 26.90 |
| ATOM | 1188 | N | THR | 1001 | 5.850 | 39.495 | 26.732 | 1.00 | 26.58 |
| ATOM | 1189 | H | THR | 1001 | 6.705 | 39.079 | 26.899 | 1.00 | 25.00 |
| ATOM | 1190 | CA | THR | 1001 | 5.508 | 39.985 | 25.378 | 1.00 | 28.24 |
| ATOM | 1191 | CB | THR | 1001 | 5.746 | 41.592 | 25.140 | 1.00 | 25.50 |
| ATOM | 1192 | OG1 | THR | 1001 | 7.175 | 41.778 | 25.114 | 1.00 | 26.31 |
| ATOM | 1193 | HG1 | THR | 1001 | 7.307 | 42.724 | 25.007 | 1.00 | 25.00 |
| ATOM | 1194 | CG2 | THR | 1001 | 5.136 | 42.535 | 26.155 | 1.00 | 20.62 |
| ATOM | 1195 | C | THR | 1001 | 6.428 | 39.270 | 24.429 | 1.00 | 28.34 |
| ATOM | 1196 | O | THR | 1001 | 7.408 | 38.651 | 24.825 | 1.00 | 28.92 |
| ATOM | 1197 | N | LEU | 1002 | 6.204 | 39.335 | 23.157 | 1.00 | 31.10 |
| ATOM | 1198 | H | LEU | 1002 | 5.393 | 39.816 | 22.929 | 1.00 | 25.00 |
| ATOM | 1199 | CA | LEU | 1002 | 6.996 | 38.707 | 22.062 | 1.00 | 31.54 |
| ATOM | 1200 | CB | LEU | 1002 | 6.135 | 39.045 | 20.924 | 1.00 | 34.22 |
| ATOM | 1201 | CG | LEU | 1002 | 6.407 | 38.956 | 19.470 | 1.00 | 38.11 |
| ATOM | 1202 | CD1 | LEU | 1002 | 6.842 | 37.562 | 18.989 | 1.00 | 41.61 |
| ATOM | 1203 | CD2 | LEU | 1002 | 5.038 | 39.381 | 18.860 | 1.00 | 40.25 |
| ATOM | 1204 | C | LEU | 1002 | 8.372 | 39.216 | 22.028 | 1.00 | 34.73 |
| ATOM | 1205 | O | LEU | 1002 | 9.280 | 38.704 | 21.372 | 1.00 | 35.66 |
| ATOM | 1206 | N | GLU | 1003 | 8.611 | 40.278 | 22.800 | 1.00 | 38.18 |
| ATOM | 1207 | H | GLU | 1003 | 7.871 | 40.668 | 23.312 | 1.00 | 25.00 |
| ATOM | 1208 | CA | GLU | 1003 | 9.935 | 40.924 | 22.842 | 1.00 | 36.59 |
| ATOM | 1209 | CB | GLU | 1003 | 9.652 | 42.204 | 23.578 | 1.00 | 40.28 |
| ATOM | 1210 | CG | GLU | 1003 | 10.772 | 43.185 | 23.158 | 1.00 | 49.51 |
| ATOM | 1211 | CD | GLU | 1003 | 10.943 | 44.527 | 23.915 | 1.00 | 52.50 |
| ATOM | 1212 | OE1 | GLU | 1003 | 9.991 | 45.033 | 24.583 | 1.00 | 52.80 |
| ATOM | 1213 | OE2 | GLU | 1003 | 12.080 | 45.009 | 23.756 | 1.00 | 54.01 |
| ATOM | 1214 | C | GLU | 1003 | 10.891 | 39.995 | 23.551 | 1.00 | 34.20 |
| ATOM | 1215 | O | GLU | 1003 | 11.925 | 39.596 | 23.025 | 1.00 | 34.55 |
| ATOM | 1216 | N | HIS | 1004 | 10.427 | 39.600 | 24.780 | 1.00 | 31.99 |
| ATOM | 1217 | H | HIS | 1004 | 9.597 | 40.048 | 25.054 | 1.00 | 25.00 |
| ATOM | 1218 | CA | HIS | 1004 | 11.045 | 38.700 | 25.828 | 1.00 | 26.95 |
| ATOM | 1219 | C | HIS | 1004 | 11.403 | 37.484 | 25.159 | 1.00 | 21.98 |
| ATOM | 1220 | O | HIS | 1004 | 12.537 | 37.226 | 24.882 | 1.00 | 19.89 |
| ATOM | 1221 | CB | HIS | 1004 | 10.007 | 38.479 | 26.863 | 1.00 | 30.00 |
| ATOM | 1222 | CG | HIS | 1004 | 9.784 | 39.667 | 27.635 | 1.00 | 34.94 |
| ATOM | 1223 | ND1 | HIS | 1004 | 10.677 | 40.250 | 28.432 | 1.00 | 36.29 |
| ATOM | 1224 | HD1 | HIS | 1004 | 11.638 | 40.162 | 28.512 | 1.00 | 25.00 |
| ATOM | 1225 | CD2 | HIS | 1004 | 8.510 | 40.160 | 27.940 | 1.00 | 40.01 |
| ATOM | 1226 | NE2 | HIS | 1004 | 8.589 | 41.040 | 28.963 | 1.00 | 39.46 |
| ATOM | 1227 | CE1 | HIS | 1004 | 9.932 | 41.073 | 29.236 | 1.00 | 39.90 |
| ATOM | 1228 | N | LEU | 1005 | 10.358 | 36.908 | 24.607 | 1.00 | 18.57 |
| ATOM | 1229 | H | LEU | 1005 | 9.488 | 37.354 | 24.690 | 1.00 | 25.00 |
| ATOM | 1230 | CA | LEU | 1005 | 10.420 | 35.675 | 23.787 | 1.00 | 16.30 |
| ATOM | 1231 | CB | LEU | 1005 | 9.015 | 35.385 | 23.169 | 1.00 | 15.28 |
| ATOM | 1232 | CG | LEU | 1005 | 8.078 | 34.541 | 24.044 | 1.00 | 14.59 |
| ATOM | 1233 | CD1 | LEU | 1005 | 7.852 | 35.030 | 25.413 | 1.00 | 15.50 |
| ATOM | 1234 | CD2 | LEU | 1005 | 6.729 | 34.655 | 23.521 | 1.00 | 13.43 |
| ATOM | 1235 | C | LEU | 1005 | 11.466 | 35.772 | 22.756 | 1.00 | 17.98 |
| ATOM | 1236 | O | LEU | 1005 | 12.279 | 34.866 | 22.581 | 1.00 | 23.32 |
| ATOM | 1237 | N | ILE | 1006 | 11.641 | 36.790 | 21.988 | 1.00 | 20.25 |
| ATOM | 1238 | H | ILE | 1006 | 11.134 | 37.613 | 22.123 | 1.00 | 25.00 |
| ATOM | 1239 | CA | ILE | 1006 | 12.803 | 36.716 | 21.041 | 1.00 | 20.94 |
| ATOM | 1240 | CB | ILE | 1006 | 12.751 | 37.826 | 19.994 | 1.00 | 23.73 |
| ATOM | 1241 | CG2 | ILE | 1006 | 13.727 | 37.376 | 18.895 | 1.00 | 22.08 |
| ATOM | 1242 | CG1 | ILE | 1006 | 11.305 | 38.082 | 19.466 | 1.00 | 25.59 |
| ATOM | 1243 | CD1 | ILE | 1006 | 11.069 | 39.126 | 18.311 | 1.00 | 25.20 |
| ATOM | 1244 | C | ILE | 1006 | 14.133 | 36.920 | 21.791 | 1.00 | 21.52 |
| ATOM | 1245 | O | ILE | 1006 | 15.282 | 36.604 | 21.320 | 1.00 | 22.23 |
| ATOM | 1246 | N | CYS | 1007 | 13.995 | 37.609 | 22.966 | 1.00 | 20.01 |
| ATOM | 1247 | H | CYS | 1007 | 13.093 | 37.915 | 23.199 | 1.00 | 25.00 |
| ATOM | 1248 | CA | CYS | 1007 | 15.168 | 37.930 | 23.854 | 1.00 | 20.59 |
| ATOM | 1249 | CB | CYS | 1007 | 14.882 | 38.903 | 25.214 | 1.00 | 16.76 |
| ATOM | 1250 | SG | CYS | 1007 | 16.553 | 39.639 | 25.409 | 1.00 | 22.27 |
| ATOM | 1251 | C | CYS | 1007 | 15.617 | 36.546 | 24.308 | 1.00 | 18.11 |
| ATOM | 1252 | O | CYS | 1007 | 16.646 | 36.144 | 23.785 | 1.00 | 21.75 |
| ATOM | 1253 | N | TYR | 1008 | 14.850 | 35.715 | 25.104 | 1.00 | 15.16 |
| ATOM | 1254 | H | TYR | 1008 | 13.978 | 36.074 | 25.344 | 1.00 | 25.00 |
| ATOM | 1255 | CA | TYR | 1008 | 15.190 | 34.299 | 25.600 | 1.00 | 9.03 |
| ATOM | 1256 | CB | TYR | 1008 | 13.882 | 33.827 | 26.069 | 1.00 | 6.13 |
| ATOM | 1257 | CG | TYR | 1008 | 13.329 | 34.597 | 27.193 | 1.00 | 2.61 |
| ATOM | 1258 | CD1 | TYR | 1008 | 11.989 | 34.525 | 27.380 | 1.00 | 3.77 |
| ATOM | 1259 | CE1 | TYR | 1008 | 11.355 | 35.152 | 28.439 | 1.00 | 7.46 |
| ATOM | 1260 | CD2 | TYR | 1008 | 14.034 | 35.364 | 28.093 | 1.00 | 4.41 |
| ATOM | 1261 | CE2 | TYR | 1008 | 13.411 | 36.003 | 29.191 | 1.00 | 5.86 |
| ATOM | 1262 | CZ | TYR | 1008 | 12.056 | 35.894 | 29.343 | 1.00 | 8.77 |
| ATOM | 1263 | OH | TYR | 1008 | 11.314 | 36.513 | 30.373 | 1.00 | 13.26 |
| ATOM | 1264 | HH | TYR | 1008 | 10.370 | 36.325 | 30.382 | 1.00 | 25.00 |
| ATOM | 1265 | C | TYR | 1008 | 15.799 | 33.469 | 24.470 | 1.00 | 11.40 |
| ATOM | 1266 | O | TYR | 1008 | 16.969 | 33.135 | 24.437 | 1.00 | 12.48 |
| ATOM | 1267 | N | SER | 1009 | 15.194 | 33.422 | 23.301 | 1.00 | 13.91 |
| ATOM | 1268 | H | SER | 1009 | 14.336 | 33.876 | 23.236 | 1.00 | 25.00 |
| ATOM | 1269 | CA | SER | 1009 | 15.641 | 32.696 | 22.077 | 1.00 | 15.24 |
| ATOM | 1270 | CB | SER | 1009 | 14.620 | 32.946 | 20.984 | 1.00 | 15.99 |
| ATOM | 1271 | OG | SER | 1009 | 13.285 | 32.786 | 21.486 | 1.00 | 17.04 |
| ATOM | 1272 | HG | SER | 1009 | 12.646 | 32.969 | 20.804 | 1.00 | 25.00 |
| ATOM | 1273 | C | SER | 1009 | 16.989 | 33.162 | 21.692 | 1.00 | 19.41 |
| ATOM | 1274 | O | SER | 1009 | 17.944 | 32.359 | 21.555 | 1.00 | 19.76 |
| ATOM | 1275 | N | PHE | 1010 | 17.116 | 34.502 | 21.433 | 1.00 | 21.84 |
| ATOM | 1276 | H | PHE | 1010 | 16.338 | 35.072 | 21.532 | 1.00 | 25.00 |
| ATOM | 1277 | CA | PHE | 1010 | 18.480 | 35.132 | 21.155 | 1.00 | 22.03 |
| ATOM | 1278 | CB | PHE | 1010 | 18.369 | 36.683 | 21.356 | 1.00 | 21.52 |
| ATOM | 1279 | CG | PHE | 1010 | 19.724 | 37.263 | 21.107 | 1.00 | 24.69 |
| ATOM | 1280 | CD1 | PHE | 1010 | 20.343 | 38.024 | 22.130 | 1.00 | 23.08 |
| ATOM | 1281 | CD2 | PHE | 1010 | 20.337 | 37.167 | 19.810 | 1.00 | 25.60 |
| ATOM | 1282 | CE1 | PHE | 1010 | 21.571 | 38.679 | 21.793 | 1.00 | 24.48 |
| ATOM | 1283 | CE2 | PHE | 1010 | 21.562 | 37.837 | 19.495 | 1.00 | 27.05 |
| ATOM | 1284 | CZ | PHE | 1010 | 22.177 | 38.608 | 20.497 | 1.00 | 25.56 |
| ATOM | 1285 | C | PHE | 1010 | 19.516 | 34.584 | 22.260 | 1.00 | 21.86 |
| ATOM | 1286 | O | PHE | 1010 | 20.531 | 33.903 | 22.013 | 1.00 | 18.72 |
| ATOM | 1287 | N | GLN | 1011 | 19.187 | 34.812 | 23.582 | 1.00 | 20.28 |
| ATOM | 1288 | H | GLN | 1011 | 18.318 | 35.220 | 23.779 | 1.00 | 25.00 |
| ATOM | 1289 | CA | GLN | 1011 | 20.052 | 34.419 | 24.699 | 1.00 | 21.13 |
| ATOM | 1290 | CB | GLN | 1011 | 19.287 | 34.876 | 25.978 | 1.00 | 21.09 |
| ATOM | 1291 | CG | GLN | 1011 | 19.216 | 36.430 | 26.135 | 1.00 | 20.85 |
| ATOM | 1292 | CD | GLN | 1011 | 18.649 | 36.623 | 27.497 | 1.00 | 19.91 |
| ATOM | 1293 | OE1 | GLN | 1011 | 19.190 | 36.291 | 28.536 | 1.00 | 27.17 |
| ATOM | 1294 | NE2 | GLN | 1011 | 17.412 | 36.978 | 27.616 | 1.00 | 22.53 |
| ATOM | 1295 | HE21 | GLN | 1011 | 16.817 | 37.128 | 26.859 | 1.00 | 25.00 |
| ATOM | 1296 | HE22 | GLN | 1011 | 17.061 | 37.098 | 28.517 | 1.00 | 25.00 |
| ATOM | 1297 | C | GLN | 1011 | 20.430 | 32.909 | 24.658 | 1.00 | 21.79 |
| ATOM | 1298 | O | GLN | 1011 | 21.623 | 32.554 | 24.681 | 1.00 | 22.26 |
| ATOM | 1299 | N | VAL | 1012 | 19.466 | 31.978 | 24.578 | 1.00 | 20.36 |
| ATOM | 1300 | H | VAL | 1012 | 18.545 | 32.264 | 24.511 | 1.00 | 25.00 |
| ATOM | 1301 | CA | VAL | 1012 | 19.847 | 30.561 | 24.487 | 1.00 | 20.58 |
| ATOM | 1302 | CB | VAL | 1012 | 18.596 | 29.730 | 24.367 | 1.00 | 21.56 |
| ATOM | 1303 | CG1 | VAL | 1012 | 19.075 | 28.295 | 24.528 | 1.00 | 23.59 |
| ATOM | 1304 | CG2 | VAL | 1012 | 17.645 | 29.906 | 25.544 | 1.00 | 21.17 |
| ATOM | 1305 | C | VAL | 1012 | 20.769 | 30.374 | 23.304 | 1.00 | 19.42 |
| ATOM | 1306 | O | VAL | 1012 | 21.835 | 29.802 | 23.405 | 1.00 | 21.92 |
| ATOM | 1307 | N | ALA | 1013 | 20.558 | 31.088 | 22.217 | 1.00 | 21.02 |
| ATOM | 1308 | H | ALA | 1013 | 19.751 | 31.667 | 22.202 | 1.00 | 25.00 |
| ATOM | 1309 | CA | ALA | 1013 | 21.436 | 30.957 | 21.028 | 1.00 | 19.71 |
| ATOM | 1310 | CB | ALA | 1013 | 20.833 | 31.754 | 19.942 | 1.00 | 17.77 |
| ATOM | 1311 | C | ALA | 1013 | 22.786 | 31.414 | 21.294 | 1.00 | 21.10 |
| ATOM | 1312 | O | ALA | 1013 | 23.794 | 30.849 | 20.884 | 1.00 | 21.39 |
| ATOM | 1313 | N | LYS | 1014 | 22.916 | 32.466 | 22.031 | 1.00 | 22.32 |
| ATOM | 1314 | H | LYS | 1014 | 22.111 | 32.864 | 22.407 | 1.00 | 25.00 |
| ATOM | 1315 | CA | LYS | 1014 | 24.279 | 32.952 | 22.322 | 1.00 | 22.07 |
| ATOM | 1316 | CB | LYS | 1014 | 23.995 | 34.430 | 22.867 | 1.00 | 25.93 |
| ATOM | 1317 | CG | LYS | 1014 | 24.245 | 35.510 | 21.744 | 1.00 | 26.62 |
| ATOM | 1318 | CD | LYS | 1014 | 25.242 | 36.634 | 22.112 | 1.00 | 29.40 |
| ATOM | 1319 | CE | LYS | 1014 | 26.802 | 36.344 | 22.120 | 1.00 | 33.74 |
| ATOM | 1320 | NZ | LYS | 1014 | 27.609 | 35.741 | 23.261 | 1.00 | 30.83 |
| ATOM | 1321 | HZ1 | LYS | 1014 | 27.138 | 34.860 | 23.545 | 1.00 | 25.00 |
| ATOM | 1322 | HZ2 | LYS | 1014 | 27.686 | 36.394 | 24.066 | 1.00 | 25.00 |
| ATOM | 1323 | HZ3 | LYS | 1014 | 28.545 | 35.535 | 22.874 | 1.00 | 25.00 |
| ATOM | 1324 | C | LYS | 1014 | 25.010 | 31.921 | 23.242 | 1.00 | 21.66 |
| ATOM | 1325 | O | LYS | 1014 | 26.190 | 31.540 | 23.028 | 1.00 | 16.43 |
| ATOM | 1326 | N | GLY | 1015 | 24.253 | 31.460 | 24.305 | 1.00 | 21.50 |
| ATOM | 1327 | H | GLY | 1015 | 23.330 | 31.783 | 24.322 | 1.00 | 25.00 |
| ATOM | 1328 | CA | GLY | 1015 | 24.718 | 30.390 | 25.269 | 1.00 | 21.58 |
| ATOM | 1329 | C | GLY | 1015 | 25.228 | 29.070 | 24.500 | 1.00 | 23.60 |
| ATOM | 1330 | O | GLY | 1015 | 26.390 | 28.607 | 24.615 | 1.00 | 21.95 |
| ATOM | 1331 | N | MET | 1016 | 24.339 | 28.453 | 23.629 | 1.00 | 21.75 |
| ATOM | 1332 | H | MET | 1016 | 23.404 | 28.738 | 23.631 | 1.00 | 25.00 |
| ATOM | 1333 | CA | MET | 1016 | 24.686 | 27.281 | 22.819 | 1.00 | 20.82 |
| ATOM | 1334 | CB | MET | 1016 | 23.591 | 26.867 | 22.014 | 1.00 | 25.04 |
| ATOM | 1335 | CG | MET | 1016 | 22.374 | 26.344 | 22.836 | 1.00 | 26.53 |
| ATOM | 1336 | SD | MET | 1016 | 23.009 | 25.128 | 23.975 | 1.00 | 28.20 |
| ATOM | 1337 | CE | MET | 1016 | 23.911 | 23.845 | 22.990 | 1.00 | 33.62 |
| ATOM | 1338 | C | MET | 1016 | 25.776 | 27.584 | 21.912 | 1.00 | 24.61 |
| ATOM | 1339 | O | MET | 1016 | 26.670 | 26.822 | 21.633 | 1.00 | 29.24 |
| ATOM | 1340 | N | GLU | 1017 | 25.782 | 28.712 | 21.283 | 1.00 | 28.84 |
| ATOM | 1341 | H | GLU | 1017 | 25.020 | 29.327 | 21.394 | 1.00 | 25.00 |
| ATOM | 1342 | CA | GLU | 1017 | 26.945 | 29.115 | 20.428 | 1.00 | 26.22 |
| ATOM | 1343 | CB | GLU | 1017 | 26.642 | 30.547 | 19.895 | 1.00 | 27.35 |
| ATOM | 1344 | CG | GLU | 1017 | 27.949 | 31.164 | 19.351 | 1.00 | 30.88 |
| ATOM | 1345 | CD | GLU | 1017 | 27.932 | 32.642 | 18.985 | 1.00 | 34.09 |
| ATOM | 1346 | OE1 | GLU | 1017 | 27.755 | 33.552 | 19.838 | 1.00 | 32.99 |
| ATOM | 1347 | OE2 | GLU | 1017 | 28.191 | 32.835 | 17.798 | 1.00 | 34.33 |
| ATOM | 1348 | C | GLU | 1017 | 28.270 | 29.096 | 21.287 | 1.00 | 25.43 |
| ATOM | 1349 | O | GLU | 1017 | 29.341 | 28.571 | 20.937 | 1.00 | 24.24 |
| ATOM | 1350 | N | PHE | 1018 | 28.235 | 29.758 | 22.488 | 1.00 | 25.50 |
| ATOM | 1351 | H | PHE | 1018 | 27.409 | 30.216 | 22.734 | 1.00 | 25.00 |
| ATOM | 1352 | CA | PHE | 1018 | 29.391 | 29.763 | 23.438 | 1.00 | 25.56 |
| ATOM | 1353 | CB | PHE | 1018 | 29.035 | 30.318 | 24.817 | 1.00 | 22.17 |
| ATOM | 1354 | CG | PHE | 1018 | 30.127 | 30.232 | 25.793 | 1.00 | 21.51 |
| ATOM | 1355 | CD1 | PHE | 1018 | 29.871 | 29.597 | 26.995 | 1.00 | 21.46 |
| ATOM | 1356 | CD2 | PHE | 1018 | 31.390 | 30.714 | 25.531 | 1.00 | 17.70 |
| ATOM | 1357 | CE1 | PHE | 1018 | 30.883 | 29.443 | 27.907 | 1.00 | 15.64 |
| ATOM | 1358 | CE2 | PHE | 1018 | 32.421 | 30.560 | 26.464 | 1.00 | 16.81 |
| ATOM | 1359 | CZ | PHE | 1018 | 32.168 | 29.912 | 27.655 | 1.00 | 16.54 |
| ATOM | 1360 | C | PHE | 1018 | 29.717 | 28.283 | 23.693 | 1.00 | 27.54 |
| ATOM | 1361 | O | PHE | 1018 | 30.871 | 27.942 | 23.506 | 1.00 | 28.16 |
| ATOM | 1362 | N | LEU | 1019 | 28.751 | 27.429 | 24.123 | 1.00 | 25.20 |
| ATOM | 1363 | H | LEU | 1019 | 27.879 | 27.816 | 24.252 | 1.00 | 25.00 |
| ATOM | 1364 | CA | LEU | 1019 | 28.977 | 26.001 | 24.425 | 1.00 | 25.82 |
| ATOM | 1365 | CB | LEU | 1019 | 27.567 | 25.364 | 24.784 | 1.00 | 22.92 |
| ATOM | 1366 | CG | LEU | 1019 | 27.244 | 25.710 | 26.267 | 1.00 | 18.39 |
| ATOM | 1367 | CD1 | LEU | 1019 | 26.032 | 25.048 | 26.855 | 1.00 | 14.71 |
| ATOM | 1368 | CD2 | LEU | 1019 | 28.369 | 25.064 | 27.067 | 1.00 | 21.21 |
| ATOM | 1369 | C | LEU | 1019 | 29.681 | 25.347 | 23.290 | 1.00 | 27.06 |
| ATOM | 1370 | O | LEU | 1019 | 30.832 | 24.933 | 23.303 | 1.00 | 26.86 |
| ATOM | 1371 | N | ALA | 1020 | 29.056 | 25.399 | 22.167 | 1.00 | 29.66 |
| ATOM | 1372 | H | ALA | 1020 | 28.189 | 25.858 | 22.126 | 1.00 | 25.00 |
| ATOM | 1373 | CA | ALA | 1020 | 29.668 | 24.835 | 20.884 | 1.00 | 33.24 |
| ATOM | 1374 | CB | ALA | 1020 | 28.941 | 25.375 | 19.625 | 1.00 | 32.45 |
| ATOM | 1375 | C | ALA | 1020 | 31.148 | 25.151 | 20.645 | 1.00 | 35.18 |
| ATOM | 1376 | O | ALA | 1020 | 31.980 | 24.365 | 20.202 | 1.00 | 37.67 |
| ATOM | 1377 | N | SER | 1021 | 31.523 | 26.386 | 20.884 | 1.00 | 38.55 |
| ATOM | 1378 | H | SER | 1021 | 30.883 | 27.001 | 21.308 | 1.00 | 25.00 |
| ATOM | 1379 | CA | SER | 1021 | 32.921 | 26.843 | 20.673 | 1.00 | 38.05 |
| ATOM | 1380 | CB | SER | 1021 | 32.927 | 28.437 | 20.874 | 1.00 | 37.42 |
| ATOM | 1381 | OG | SER | 1021 | 32.998 | 28.967 | 22.230 | 1.00 | 33.65 |
| ATOM | 1382 | HG | SER | 1021 | 33.049 | 29.926 | 22.190 | 1.00 | 25.00 |
| ATOM | 1383 | C | SER | 1021 | 33.925 | 26.145 | 21.600 | 1.00 | 38.01 |
| ATOM | 1384 | O | SER | 1021 | 35.116 | 25.942 | 21.292 | 1.00 | 39.52 |
| ATOM | 1385 | N | ARG | 1022 | 33.389 | 25.850 | 22.789 | 1.00 | 35.96 |
| ATOM | 1386 | H | ARG | 1022 | 32.454 | 26.050 | 22.970 | 1.00 | 25.00 |
| ATOM | 1387 | CA | ARG | 1022 | 34.161 | 25.235 | 23.838 | 1.00 | 36.56 |
| ATOM | 1388 | CB | ARG | 1022 | 33.409 | 25.397 | 25.134 | 1.00 | 34.23 |
| ATOM | 1389 | CG | ARG | 1022 | 33.223 | 26.909 | 25.400 | 1.00 | 32.65 |
| ATOM | 1390 | CD | ARG | 1022 | 34.274 | 27.724 | 26.151 | 1.00 | 29.05 |
| ATOM | 1391 | NE | ARG | 1022 | 35.448 | 27.553 | 25.398 | 1.00 | 27.20 |
| ATOM | 1392 | HE | AEG | 1022 | 35.417 | 27.783 | 24.448 | 1.00 | 25.00 |
| ATOM | 1393 | CZ | ARG | 1022 | 36.520 | 27.056 | 25.909 | 1.00 | 28.23 |
| ATOM | 1394 | NH1 | ARG | 1022 | 37.513 | 26.902 | 25.051 | 1.00 | 31.11 |
| ATOM | 1395 | HH11 | ARG | 1022 | 37.370 | 27.128 | 24.090 | 1.00 | 25.00 |
| ATOM | 1396 | HH12 | ARG | 1022 | 38.403 | 26.524 | 25.312 | 1.00 | 25.00 |
| ATOM | 1397 | NH2 | ARG | 1022 | 36.655 | 26.761 | 27.166 | 1.00 | 25.35 |
| ATOM | 1398 | HH21 | ARG | 1022 | 35.903 | 26.948 | 27.783 | 1.00 | 25.00 |
| ATOM | 1399 | HH22 | ARG | 1022 | 37.527 | 26.404 | 27.482 | 1.00 | 25.00 |
| ATOM | 1400 | C | ARG | 1022 | 34.474 | 23.736 | 23.585 | 1.00 | 38.64 |
| ATOM | 1401 | O | ARG | 1022 | 35.091 | 23.128 | 24.360 | 1.00 | 39.29 |
| ATOM | 1402 | N | LYS | 1023 | 33.909 | 23.297 | 22.412 | 1.00 | 39.78 |
| ATOM | 1403 | H | LYS | 1023 | 33.445 | 24.057 | 22.082 | 1.00 | 25.00 |
| ATOM | 1404 | CA | LYS | 1023 | 33.739 | 22.048 | 21.633 | 1.00 | 39.06 |
| ATOM | 1405 | CB | LYS | 1023 | 35.056 | 21.243 | 21.472 | 1.00 | 38.70 |
| ATOM | 1406 | CG | LYS | 1023 | 36.022 | 21.938 | 20.636 | 1.00 | 43.18 |
| ATOM | 1407 | CD | LYS | 1023 | 37.048 | 22.666 | 21.540 | 1.00 | 50.71 |
| ATOM | 1408 | CE | LYS | 1023 | 38.410 | 23.052 | 20.827 | 1.00 | 55.05 |
| ATOM | 1409 | NZ | LYS | 1023 | 38.072 | 23.610 | 19.464 | 1.00 | 62.01 |
| ATOM | 1410 | HZ1 | LYS | 1023 | 37.262 | 24.265 | 19.526 | 1.00 | 25.00 |
| ATOM | 1411 | HZ2 | LYS | 1023 | 37.842 | 22.835 | 18.809 | 1.00 | 25.00 |
| ATOM | 1412 | HZ3 | LYS | 1023 | 38.886 | 24.121 | 19.069 | 1.00 | 25.00 |
| ATOM | 1413 | C | LYS | 1023 | 32.745 | 21.234 | 22.475 | 1.00 | 38.01 |
| ATOM | 1414 | O | LYS | 1023 | 32.973 | 20.071 | 22.776 | 1.00 | 41.13 |
| ATOM | 1415 | N | CYS | 1024 | 31.496 | 21.645 | 22.695 | 1.00 | 33.05 |
| ATOM | 1416 | H | CYS | 1024 | 31.119 | 22.436 | 22.224 | 1.00 | 25.00 |
| ATOM | 1417 | CA | CYS | 1024 | 30.737 | 20.825 | 23.637 | 1.00 | 31.74 |
| ATOM | 1418 | CB | CYS | 1024 | 30.559 | 21.423 | 25.040 | 1.00 | 31.61 |
| ATOM | 1419 | SG | CYS | 1024 | 32.069 | 21.406 | 26.098 | 1.00 | 44.63 |
| ATOM | 1420 | C | CYS | 1024 | 29.447 | 20.611 | 23.212 | 1.00 | 32.42 |
| ATOM | 1421 | O | CYS | 1024 | 28.951 | 21.387 | 22.453 | 1.00 | 34.99 |
| ATOM | 1422 | N | ILE | 1025 | 28.801 | 19.626 | 23.741 | 1.00 | 33.27 |
| ATOM | 1423 | H | ILE | 1025 | 29.186 | 19.089 | 24.460 | 1.00 | 25.00 |
| ATOM | 1424 | CA | ILE | 1025 | 27.479 | 19.367 | 23.292 | 1.00 | 33.30 |
| ATOM | 1425 | CB | ILE | 1025 | 27.711 | 18.420 | 21.999 | 1.00 | 37.18 |
| ATOM | 1426 | CG2 | ILE | 1025 | 29.134 | 18.231 | 21.495 | 1.00 | 38.21 |
| ATOM | 1427 | CG1 | ILE | 1025 | 27.301 | 17.037 | 22.316 | 1.00 | 42.22 |
| ATOM | 1428 | CD1 | ILE | 1025 | 25.990 | 17.068 | 21.406 | 1.00 | 45.78 |
| ATOM | 1429 | C | ILE | 1025 | 26.651 | 18.887 | 24.456 | 1.00 | 30.64 |
| ATOM | 1430 | O | ILE | 1025 | 27.079 | 17.964 | 25.133 | 1.00 | 34.55 |
| ATOM | 1431 | N | HIS | 1026 | 25.507 | 19.588 | 24.620 | 1.00 | 26.01 |
| ATOM | 1432 | H | HIS | 1026 | 25.350 | 20.207 | 23.874 | 1.00 | 25.00 |
| ATOM | 1433 | CA | HIS | 1026 | 24.495 | 19.471 | 25.665 | 1.00 | 26.50 |
| ATOM | 1434 | C | HIS | 1026 | 23.663 | 18.160 | 25.726 | 1.00 | 29.77 |
| ATOM | 1435 | O | HIS | 1026 | 23.490 | 17.556 | 26.805 | 1.00 | 34.32 |
| ATOM | 1436 | CB | HIS | 1026 | 23.542 | 20.681 | 25.568 | 1.00 | 20.89 |
| ATOM | 1437 | CG | HIS | 1026 | 22.694 | 20.759 | 26.819 | 1.00 | 20.25 |
| ATOM | 1438 | ND1 | HIS | 1026 | 21.545 | 20.070 | 27.125 | 1.00 | 23.32 |
| ATOM | 1439 | CE1 | HIS | 1026 | 20.917 | 20.526 | 28.297 | 1.00 | 19.96 |
| ATOM | 1440 | CD2 | HIS | 1026 | 22.766 | 21.671 | 27.870 | 1.00 | 21.28 |
| ATOM | 1441 | NE2 | HIS | 1026 | 21.706 | 21.533 | 28.728 | 1.00 | 22.34 |
| ATOM | 1442 | HE2 | HIS | 1026 | 21.651 | 21.956 | 29.624 | 1.00 | 25.00 |
| ATOM | 1443 | N | ARG | 1027 | 23.075 | 17.700 | 24.586 | 1.00 | 29.80 |
| ATOM | 1444 | H | ARG | 1027 | 23.206 | 18.200 | 23.749 | 1.00 | 25.00 |
| ATOM | 1445 | CA | ARG | 1027 | 22.284 | 16.434 | 24.464 | 1.00 | 28.23 |
| ATOM | 1446 | CB | ARG | 1027 | 23.218 | 15.228 | 24.949 | 1.00 | 32.11 |
| ATOM | 1447 | CG | ARG | 1027 | 24.468 | 14.937 | 24.056 | 1.00 | 34.57 |
| ATOM | 1448 | CD | ARG | 1027 | 25.467 | 13.966 | 24.669 | 1.00 | 40.62 |
| ATOM | 1449 | NE | ARG | 1027 | 26.816 | 14.534 | 24.457 | 1.00 | 48.49 |
| ATOM | 1450 | HE | ARG | 1027 | 27.007 | 15.475 | 24.651 | 1.00 | 25.00 |
| ATOM | 1451 | CZ | ARG | 1027 | 27.867 | 13.790 | 24.040 | 1.00 | 52.05 |
| ATOM | 1452 | NH1 | ARG | 1027 | 29.092 | 14.379 | 23.869 | 1.00 | 54.51 |
| ATOM | 1453 | HH11 | ARG | 1027 | 29.221 | 15.350 | 24.084 | 1.00 | 25.00 |
| ATOM | 1454 | HH12 | ARG | 1027 | 29.873 | 13.823 | 23.550 | 1.00 | 25.00 |
| ATOM | 1455 | NH2 | ARG | 1027 | 27.726 | 12.474 | 23.753 | 1.00 | 53.39 |
| ATOM | 1456 | HH21 | ARG | 1027 | 26.841 | 12.008 | 23.822 | 1.00 | 25.00 |
| ATOM | 1457 | HH22 | ARG | 1027 | 28.525 | 11.959 | 23.418 | 1.00 | 25.00 |
| ATOM | 1458 | C | ARG | 1027 | 20.946 | 16.442 | 25.108 | 1.00 | 25.61 |
| ATOM | 1459 | O | ARG | 1027 | 20.075 | 15.599 | 24.834 | 1.00 | 26.41 |
| ATOM | 1460 | N | ASP | 1028 | 20.712 | 17.370 | 25.995 | 1.00 | 22.28 |
| ATOM | 1461 | H | ASP | 1028 | 21.422 | 17.928 | 26.357 | 1.00 | 25.00 |
| ATOM | 1462 | CA | ASP | 1028 | 19.309 | 17.416 | 26.470 | 1.00 | 24.88 |
| ATOM | 1463 | CB | ASP | 1028 | 19.088 | 16.831 | 27.822 | 1.00 | 21.12 |
| ATOM | 1464 | CG | ASP | 1028 | 17.590 | 16.694 | 28.223 | 1.00 | 25.13 |
| ATOM | 1465 | OD1 | ASP | 1028 | 17.424 | 16.372 | 29.386 | 1.00 | 27.20 |
| ATOM | 1466 | OD2 | ASP | 1028 | 16.585 | 16.825 | 27.478 | 1.00 | 21.85 |
| ATOM | 1467 | C | ASP | 1028 | 18.774 | 18.862 | 26.557 | 1.00 | 26.41 |
| ATOM | 1468 | O | ASP | 1028 | 18.101 | 19.262 | 27.525 | 1.00 | 29.04 |
| ATOM | 1469 | N | LEU | 1029 | 19.048 | 19.665 | 25.504 | 1.00 | 26.14 |
| ATOM | 1470 | H | LEU | 1029 | 19.573 | 19.261 | 24.775 | 1.00 | 25.00 |
| ATOM | 1471 | CA | LEU | 1029 | 18.664 | 21.071 | 25.412 | 1.00 | 25.63 |
| ATOM | 1472 | CB | LEU | 1029 | 19.410 | 21.607 | 24.247 | 1.00 | 23.25 |
| ATOM | 1473 | CG | LEU | 1029 | 19.097 | 23.084 | 24.001 | 1.00 | 23.98 |
| ATOM | 1474 | CD1 | LEU | 1029 | 19.059 | 24.033 | 25.202 | 1.00 | 19.72 |
| ATOM | 1475 | CD2 | LEU | 1029 | 20.188 | 23.351 | 22.985 | 1.00 | 21.04 |
| ATOM | 1476 | C | LEU | 1029 | 17.172 | 21.184 | 25.268 | 1.00 | 28.53 |
| ATOM | 1477 | O | LEU | 1029 | 16.536 | 20.573 | 24.416 | 1.00 | 29.06 |
| ATOM | 1478 | N | ALA | 1030 | 16.530 | 21.910 | 26.154 | 1.00 | 27.58 |
| ATOM | 1479 | H | ALA | 1030 | 17.045 | 22.255 | 26.919 | 1.00 | 25.00 |
| ATOM | 1480 | CA | ALA | 1030 | 15.073 | 21.993 | 26.122 | 1.00 | 24.12 |
| ATOM | 1481 | CB | ALA | 1030 | 14.473 | 20.711 | 26.755 | 1.00 | 22.40 |
| ATOM | 1482 | C | ALA | 1030 | 14.800 | 23.142 | 27.003 | 1.00 | 24.69 |
| ATOM | 1483 | O | ALA | 1030 | 15.715 | 23.648 | 27.634 | 1.00 | 25.53 |
| ATOM | 1484 | N | ALA | 1031 | 13.580 | 23.576 | 27.130 | 1.00 | 24.68 |
| ATOM | 1485 | H | ALA | 1031 | 12.911 | 23.163 | 26.571 | 1.00 | 25.00 |
| ATOM | 1486 | CA | ALA | 1031 | 13.214 | 24.700 | 27.999 | 1.00 | 23.50 |
| ATOM | 1487 | CB | ALA | 1031 | 11.929 | 25.306 | 27.480 | 1.00 | 25.13 |
| ATOM | 1488 | C | ALA | 1031 | 12.981 | 24.198 | 29.418 | 1.00 | 24.64 |
| ATOM | 1489 | O | ALA | 1031 | 12.525 | 24.960 | 30.234 | 1.00 | 26.49 |
| ATOM | 1490 | N | ARG | 1032 | 13.012 | 22.892 | 29.757 | 1.00 | 22.29 |
| ATOM | 1491 | H | ARG | 1032 | 13.031 | 22.264 | 29.013 | 1.00 | 25.00 |
| ATOM | 1492 | CA | ARG | 1032 | 12.864 | 22.442 | 31.138 | 1.00 | 17.04 |
| ATOM | 1493 | CB | ARG | 1032 | 12.510 | 20.954 | 31.245 | 1.00 | 13.25 |
| ATOM | 1494 | CG | ARG | 1032 | 13.458 | 19.993 | 30.548 | 1.00 | 13.62 |
| ATOM | 1495 | CD | ARG | 1032 | 13.422 | 18.468 | 30.582 | 1.00 | 12.87 |
| ATOM | 1496 | NE | ARG | 1032 | 12.559 | 18.295 | 29.444 | 1.00 | 15.68 |
| ATOM | 1497 | HE | ARG | 1032 | 11.611 | 18.499 | 29.491 | 1.00 | 25.00 |
| ATOM | 1498 | CZ | ARG | 1032 | 12.978 | 17.942 | 28.274 | 1.00 | 12.03 |
| ATOM | 1499 | NH1 | ARG | 1032 | 11.988 | 17.950 | 27.440 | 1.00 | 14.06 |
| ATOM | 1500 | HH11 | ARG | 1032 | 11.050 | 18.213 | 27.686 | 1.00 | 25.00 |
| ATOM | 1501 | HH12 | ARG | 1032 | 12.184 | 17.673 | 26.499 | 1.00 | 25.00 |
| ATOM | 1502 | NH2 | ARG | 1032 | 14.219 | 17.563 | 27.923 | 1.00 | 9.68 |
| ATOM | 1503 | HH21 | ARG | 1032 | 15.015 | 17.538 | 28.546 | 1.00 | 25.00 |
| ATOM | 1504 | HH22 | ARG | 1032 | 14.350 | 17.297 | 26.968 | 1.00 | 25.00 |
| ATOM | 1505 | C | ARG | 1032 | 14.287 | 22.704 | 31.577 | 1.00 | 19.80 |
| ATOM | 1506 | O | ARG | 1032 | 14.572 | 22.938 | 32.736 | 1.00 | 22.83 |
| ATOM | 1507 | N | ASN | 1033 | 15.279 | 22.528 | 30.715 | 1.00 | 15.85 |
| ATOM | 1508 | H | ASN | 1033 | 15.064 | 22.299 | 29.796 | 1.00 | 25.00 |
| ATOM | 1509 | CA | ASN | 1033 | 16.625 | 22.897 | 31.136 | 1.00 | 15.74 |
| ATOM | 1510 | CB | ASN | 1033 | 17.652 | 21.947 | 30.355 | 1.00 | 15.87 |
| ATOM | 1511 | CG | ASN | 1033 | 17.572 | 20.632 | 30.988 | 1.00 | 15.81 |
| ATOM | 1512 | OD1 | ASN | 1033 | 17.101 | 20.466 | 32.101 | 1.00 | 17.66 |
| ATOM | 1513 | ND2 | ASN | 1033 | 17.917 | 19.587 | 30.318 | 1.00 | 16.68 |
| ATOM | 1514 | HD21 | ASN | 1033 | 18.218 | 19.632 | 29.398 | 1.00 | 25.00 |
| ATOM | 1515 | HD22 | ASN | 1033 | 17.851 | 18.745 | 30.808 | 1.00 | 25.00 |
| ATOM | 1516 | C | ASN | 1033 | 17.042 | 24.488 | 30.950 | 1.00 | 16.79 |
| ATOM | 1517 | O | ASN | 1033 | 18.217 | 24.928 | 30.812 | 1.00 | 13.15 |
| ATOM | 1518 | N | ILE | 1034 | 16.077 | 25.424 | 30.913 | 1.00 | 16.41 |
| ATOM | 1519 | H | ILE | 1034 | 15.124 | 25.204 | 31.092 | 1.00 | 25.00 |
| ATOM | 1520 | CA | ILE | 1034 | 16.542 | 26.783 | 30.705 | 1.00 | 15.12 |
| ATOM | 1521 | CB | ILE | 1034 | 16.024 | 27.418 | 29.429 | 1.00 | 19.12 |
| ATOM | 1522 | CG2 | ILE | 1034 | 16.748 | 28.760 | 29.327 | 1.00 | 22.07 |
| ATOM | 1523 | CG1 | ILE | 1034 | 16.410 | 26.742 | 28.098 | 1.00 | 11.09 |
| ATOM | 1524 | CD1 | ILE | 1034 | 17.829 | 26.722 | 27.654 | 1.00 | 8.91 |
| ATOM | 1525 | C | ILE | 1034 | 15.960 | 27.466 | 31.869 | 1.00 | 16.80 |
| ATOM | 1526 | O | ILE | 1034 | 14.817 | 27.221 | 32.206 | 1.00 | 11.75 |
| ATOM | 1527 | N | LEU | 1035 | 16.880 | 28.073 | 32.691 | 1.00 | 20.93 |
| ATOM | 1528 | H | LEU | 1035 | 17.825 | 27.915 | 32.554 | 1.00 | 25.00 |
| ATOM | 1529 | CA | LEU | 1035 | 16.382 | 28.737 | 33.906 | 1.00 | 22.74 |
| ATOM | 1530 | CB | LEU | 1035 | 17.293 | 28.330 | 35.238 | 1.00 | 21.73 |
| ATOM | 1531 | CG | LEU | 1035 | 17.609 | 26.806 | 35.514 | 1.00 | 23.45 |
| ATOM | 1532 | CD1 | LEU | 1035 | 18.895 | 26.746 | 36.360 | 1.00 | 24.02 |
| ATOM | 1533 | CD2 | LEU | 1035 | 16.428 | 26.064 | 36.092 | 1.00 | 19.48 |
| ATOM | 1534 | C | LEU | 1035 | 16.320 | 30.292 | 33.687 | 1.00 | 21.01 |
| ATOM | 1535 | O | LEU | 1035 | 17.125 | 30.917 | 33.007 | 1.00 | 21.00 |
| ATOM | 1536 | N | LEU | 1036 | 15.332 | 30.929 | 34.239 | 1.00 | 19.94 |
| ATOM | 1537 | H | LEU | 1036 | 14.716 | 30.402 | 34.785 | 1.00 | 25.00 |
| ATOM | 1538 | CA | LEU | 1036 | 15.143 | 32.353 | 34.169 | 1.00 | 19.83 |
| ATOM | 1539 | CB | LEU | 1036 | 13.631 | 32.687 | 33.778 | 1.00 | 19.83 |
| ATOM | 1540 | CG | LEU | 1036 | 13.383 | 34.214 | 33.415 | 1.00 | 20.62 |
| ATOM | 1541 | CD1 | LEU | 1036 | 14.359 | 34.761 | 32.371 | 1.00 | 16.36 |
| ATOM | 1542 | CD2 | LEU | 1036 | 11.993 | 34.307 | 32.861 | 1.00 | 19.55 |
| ATOM | 1543 | C | LEU | 1036 | 15.507 | 33.020 | 35.513 | 1.00 | 20.70 |
| ATOM | 1544 | O | LEU | 1036 | 14.890 | 32.815 | 36.574 | 1.00 | 20.34 |
| ATOM | 1545 | N | SER | 1037 | 16.673 | 33.731 | 35.468 | 1.00 | 23.76 |
| ATOM | 1546 | H | SER | 1037 | 17.137 | 33.710 | 34.588 | 1.00 | 25.00 |
| ATOM | 1547 | CA | SER | 1037 | 17.321 | 34.595 | 36.523 | 1.00 | 26.43 |
| ATOM | 1548 | CB | SER | 1037 | 18.754 | 34.670 | 36.202 | 1.00 | 23.76 |
| ATOM | 1549 | OG | SER | 1037 | 19.260 | 35.461 | 37.264 | 1.00 | 31.21 |
| ATOM | 1550 | HG | SER | 1037 | 20.222 | 35.444 | 37.408 | 1.00 | 25.00 |
| ATOM | 1551 | C | SER | 1037 | 16.730 | 36.021 | 36.543 | 1.00 | 28.96 |
| ATOM | 1552 | O | SER | 1037 | 15.690 | 36.320 | 35.950 | 1.00 | 29.18 |
| ATOM | 1553 | N | GLU | 1038 | 17.267 | 36.986 | 37.206 | 1.00 | 33.44 |
| ATOM | 1554 | H | GLU | 1038 | 18.169 | 36.858 | 37.524 | 1.00 | 25.00 |
| ATOM | 1555 | CA | GLU | 1038 | 16.656 | 38.358 | 37.227 | 1.00 | 37.79 |
| ATOM | 1556 | CB | GLU | 1038 | 17.140 | 39.328 | 38.294 | 1.00 | 44.42 |
| ATOM | 1557 | CG | GLU | 1038 | 16.325 | 39.145 | 39.602 | 1.00 | 55.87 |
| ATOM | 1558 | CD | GLU | 1038 | 17.280 | 38.380 | 40.483 | 1.00 | 63.29 |
| ATOM | 1559 | OE1 | GLU | 1038 | 17.241 | 37.143 | 40.403 | 1.00 | 70.83 |
| ATOM | 1560 | OE2 | GLU | 1038 | 18.086 | 38.996 | 41.206 | 1.00 | 68.35 |
| ATOM | 1561 | C | GLU | 1038 | 16.871 | 39.181 | 36.037 | 1.00 | 34.13 |
| ATOM | 1562 | O | GLU | 1038 | 17.763 | 38.845 | 35.267 | 1.00 | 33.56 |
| ATOM | 1563 | N | LYS | 1039 | 16.020 | 40.193 | 35.848 | 1.00 | 30.85 |
| ATOM | 1564 | H | LYS | 1039 | 15.326 | 40.431 | 36.446 | 1.00 | 25.00 |
| ATOM | 1565 | CA | LYS | 1039 | 16.222 | 41.071 | 34.731 | 1.00 | 29.12 |
| ATOM | 1566 | CB | LYS | 1039 | 17.510 | 41.929 | 35.206 | 1.00 | 36.93 |
| ATOM | 1567 | CG | LYS | 1039 | 17.268 | 43.070 | 36.296 | 1.00 | 45.01 |
| ATOM | 1568 | CD | LYS | 1039 | 16.546 | 44.423 | 35.700 | 1.00 | 50.88 |
| ATOM | 1569 | CE | LYS | 1039 | 15.240 | 44.364 | 34.734 | 1.00 | 53.62 |
| ATOM | 1570 | NZ | LYS | 1039 | 15.483 | 44.307 | 33.247 | 1.00 | 56.74 |
| ATOM | 1571 | HZ1 | LYS | 1039 | 16.214 | 43.601 | 33.053 | 1.00 | 25.00 |
| ATOM | 1572 | HZ2 | LYS | 1039 | 15.859 | 45.211 | 32.899 | 1.00 | 25.00 |
| ATOM | 1573 | HZ3 | LYS | 1039 | 14.618 | 44.077 | 32.726 | 1.00 | 25.00 |
| ATOM | 1574 | C | LYS | 1039 | 16.306 | 40.331 | 33.396 | 1.00 | 23.17 |
| ATOM | 1575 | O | LYS | 1039 | 17.159 | 40.558 | 32.603 | 1.00 | 17.05 |
| ATOM | 1576 | N | ASN | 1040 | 15.358 | 39.465 | 33.088 | 1.00 | 23.92 |
| ATOM | 1577 | H | ASN | 1040 | 14.689 | 39.321 | 33.772 | 1.00 | 25.00 |
| ATOM | 1578 | CA | ASN | 1040 | 15.285 | 38.648 | 31.873 | 1.00 | 25.79 |
| ATOM | 1579 | CB | ASN | 1040 | 14.854 | 39.455 | 30.701 | 1.00 | 30.40 |
| ATOM | 1580 | CG | ASN | 1040 | 13.451 | 39.907 | 30.880 | 1.00 | 33.72 |
| ATOM | 1581 | OD1 | ASN | 1040 | 12.487 | 39.183 | 31.088 | 1.00 | 38.65 |
| ATOM | 1582 | ND2 | ASN | 1040 | 13.274 | 41.183 | 30.869 | 1.00 | 36.83 |
| ATOM | 1583 | HD21 | ASN | 1040 | 14.042 | 41.769 | 30.760 | 1.00 | 25.00 |
| ATOM | 1584 | HD22 | ASN | 1040 | 12.375 | 41.535 | 30.981 | 1.00 | 25.00 |
| ATOM | 1585 | C | ASN | 1040 | 16.487 | 37.907 | 31.446 | 1.00 | 25.85 |
| ATOM | 1586 | O | ASN | 1040 | 16.738 | 37.773 | 30.266 | 1.00 | 28.01 |
| ATOM | 1587 | N | VAL | 1041 | 17.360 | 37.405 | 32.295 | 1.00 | 29.22 |
| ATOM | 1588 | H | VAL | 1041 | 17.251 | 37.599 | 33.238 | 1.00 | 25.00 |
| ATOM | 1589 | CA | VAL | 1041 | 18.537 | 36.587 | 31.804 | 1.00 | 28.01 |
| ATOM | 1590 | CB | VAL | 1041 | 19.856 | 36.834 | 32.579 | 1.00 | 27.95 |
| ATOM | 1591 | CG1 | VAL | 1041 | 20.943 | 36.217 | 31.691 | 1.00 | 24.89 |
| ATOM | 1592 | CG2 | VAL | 1041 | 20.134 | 38.380 | 32.870 | 1.00 | 27.12 |
| ATOM | 1593 | C | VAL | 1041 | 18.080 | 35.120 | 32.077 | 1.00 | 27.95 |
| ATOM | 1594 | O | VAL | 1041 | 17.368 | 34.774 | 33.046 | 1.00 | 26.90 |
| ATOM | 1595 | N | VAL | 1042 | 18.184 | 34.378 | 31.003 | 1.00 | 25.16 |
| ATOM | 1596 | H | VAL | 1042 | 18.583 | 34.821 | 30.241 | 1.00 | 25.00 |
| ATOM | 1597 | CA | VAL | 1042 | 17.820 | 32.970 | 30.907 | 1.00 | 21.12 |
| ATOM | 1598 | CB | VAL | 1042 | 16.921 | 33.129 | 29.674 | 1.00 | 17.71 |
| ATOM | 1599 | CG1 | VAL | 1042 | 17.675 | 32.585 | 28.445 | 1.00 | 16.43 |
| ATOM | 1600 | CG2 | VAL | 1042 | 15.532 | 32.707 | 30.064 | 1.00 | 12.78 |
| ATOM | 1601 | C | VAL | 1042 | 19.229 | 32.337 | 30.876 | 1.00 | 21.51 |
| ATOM | 1602 | O | VAL | 1042 | 20.217 | 32.817 | 30.303 | 1.00 | 20.59 |
| ATOM | 1603 | N | LYS | 1043 | 19.423 | 31.301 | 31.705 | 1.00 | 23.20 |
| ATOM | 1604 | H | LYS | 1043 | 18.686 | 31.131 | 32.306 | 1.00 | 25.00 |
| ATOM | 1605 | CA | LYS | 1043 | 20.673 | 30.427 | 31.929 | 1.00 | 20.32 |
| ATOM | 1606 | CB | LYS | 1043 | 20.948 | 30.490 | 33.499 | 1.00 | 19.77 |
| ATOM | 1607 | CG | LYS | 1043 | 22.078 | 31.497 | 33.528 | 1.00 | 15.44 |
| ATOM | 1608 | CD | LYS | 1043 | 21.344 | 32.576 | 34.187 | 1.00 | 17.89 |
| ATOM | 1609 | CE | LYS | 1043 | 22.194 | 33.816 | 33.848 | 1.00 | 15.84 |
| ATOM | 1610 | NZ | LYS | 1043 | 23.563 | 33.942 | 34.330 | 1.00 | 16.95 |
| ATOM | 1611 | HZ1 | LYS | 1043 | 24.081 | 33.043 | 34.207 | 1.00 | 25.00 |
| ATOM | 1612 | HZ2 | LYS | 1043 | 23.557 | 34.180 | 35.344 | 1.00 | 25.00 |
| ATOM | 1613 | HZ3 | LYS | 1043 | 24.074 | 34.685 | 33.790 | 1.00 | 25.00 |
| ATOM | 1614 | C | LYS | 1043 | 20.532 | 28.944 | 31.417 | 1.00 | 18.97 |
| ATOM | 1615 | O | LYS | 1043 | 19.559 | 28.233 | 31.702 | 1.00 | 16.79 |
| ATOM | 1616 | N | ILE | 1044 | 21.305 | 28.516 | 30.503 | 1.00 | 14.75 |
| ATOM | 1617 | H | ILE | 1044 | 21.954 | 29.093 | 30.078 | 1.00 | 25.00 |
| ATOM | 1618 | CA | ILE | 1044 | 21.199 | 27.157 | 30.150 | 1.00 | 16.47 |
| ATOM | 1619 | CB | ILE | 1044 | 22.088 | 26.830 | 28.968 | 1.00 | 17.35 |
| ATOM | 1620 | CG2 | ILE | 1044 | 22.221 | 25.250 | 28.847 | 1.00 | 13.49 |
| ATOM | 1621 | CG1 | ILE | 1044 | 21.541 | 27.507 | 27.715 | 1.00 | 17.70 |
| ATOM | 1622 | CD1 | ILE | 1044 | 22.667 | 27.664 | 26.673 | 1.00 | 9.28 |
| ATOM | 1623 | C | ILE | 1044 | 21.665 | 26.259 | 31.265 | 1.00 | 17.50 |
| ATOM | 1624 | O | ILE | 1044 | 22.852 | 26.326 | 31.590 | 1.00 | 15.15 |
| ATOM | 1625 | N | CYS | 1045 | 20.875 | 25.446 | 31.954 | 1.00 | 20.73 |
| ATOM | 1626 | H | CYS | 1045 | 19.898 | 25.558 | 31.907 | 1.00 | 25.00 |
| ATOM | 1627 | CA | CYS | 1045 | 21.549 | 24.487 | 32.946 | 1.00 | 21.86 |
| ATOM | 1628 | CB | CYS | 1045 | 20.969 | 24.655 | 34.269 | 1.00 | 15.99 |
| ATOM | 1629 | SG | CYS | 1045 | 19.374 | 24.021 | 34.191 | 1.00 | 21.11 |
| ATOM | 1630 | C | CYS | 1045 | 21.479 | 22.970 | 32.533 | 1.00 | 23.82 |
| ATOM | 1631 | O | CYS | 1045 | 21.554 | 22.682 | 31.335 | 1.00 | 28.59 |
| ATOM | 1632 | N | ASP | 1046 | 21.634 | 21.961 | 33.372 | 1.00 | 23.21 |
| ATOM | 1633 | H | ASP | 1046 | 21.790 | 22.193 | 34.307 | 1.00 | 25.00 |
| ATOM | 1634 | CA | ASP | 1046 | 21.504 | 20.556 | 33.023 | 1.00 | 18.69 |
| ATOM | 1635 | CB | ASP | 1046 | 22.718 | 19.839 | 32.531 | 1.00 | 20.96 |
| ATOM | 1636 | CG | ASP | 1046 | 22.237 | 18.581 | 31.674 | 1.00 | 29.52 |
| ATOM | 1637 | OD1 | ASP | 1046 | 22.831 | 18.392 | 30.626 | 1.00 | 34.52 |
| ATOM | 1638 | OD2 | ASP | 1046 | 21.280 | 17.800 | 31.891 | 1.00 | 31.53 |
| ATOM | 1639 | C | ASP | 1046 | 21.134 | 19.962 | 34.287 | 1.00 | 17.34 |
| ATOM | 1640 | O | ASP | 1046 | 21.990 | 19.984 | 35.137 | 1.00 | 17.07 |
| ATOM | 1641 | N | PHE | 1047 | 19.862 | 19.675 | 34.586 | 1.00 | 17.24 |
| ATOM | 1642 | H | PHE | 1047 | 19.129 | 19.933 | 33.986 | 1.00 | 25.00 |
| ATOM | 1643 | CA | PHE | 1047 | 19.574 | 19.020 | 35.891 | 1.00 | 20.86 |
| ATOM | 1644 | CB | PHE | 1047 | 18.117 | 18.716 | 36.126 | 1.00 | 17.07 |
| ATOM | 1645 | CG | PHE | 1047 | 17.362 | 19.934 | 36.358 | 1.00 | 18.68 |
| ATOM | 1646 | CD1 | PHE | 1047 | 17.199 | 20.865 | 35.355 | 1.00 | 20.17 |
| ATOM | 1647 | CD2 | PHE | 1047 | 16.737 | 20.127 | 37.548 | 1.00 | 16.12 |
| ATOM | 1648 | CE1 | PHE | 1047 | 16.345 | 21.964 | 35.581 | 1.00 | 21.18 |
| ATOM | 1649 | CE2 | PHE | 1047 | 15.887 | 21.200 | 37.784 | 1.00 | 20.05 |
| ATOM | 1650 | CZ | PHE | 1047 | 15.673 | 22.128 | 36.776 | 1.00 | 16.46 |
| ATOM | 1651 | C | PHE | 1047 | 20.280 | 17.670 | 36.229 | 1.00 | 24.49 |
| ATOM | 1652 | O | PHE | 1047 | 20.429 | 17.309 | 37.416 | 1.00 | 26.72 |
| ATOM | 1653 | N | GLY | 1048 | 20.811 | 16.926 | 35.241 | 1.00 | 27.85 |
| ATOM | 1654 | H | GLY | 1048 | 20.795 | 17.304 | 34.344 | 1.00 | 25.00 |
| ATOM | 1655 | CA | GLY | 1048 | 21.461 | 15.634 | 35.518 | 1.00 | 29.94 |
| ATOM | 1656 | C | GLY | 1048 | 20.513 | 14.773 | 36.365 | 1.00 | 32.44 |
| ATOM | 1657 | O | GLY | 1048 | 19.305 | 14.639 | 36.047 | 1.00 | 35.02 |
| ATOM | 1658 | N | LEU | 1049 | 21.008 | 14.241 | 37.494 | 1.00 | 30.16 |
| ATOM | 1659 | H | LEU | 1049 | 21.886 | 14.542 | 37.783 | 1.00 | 25.00 |
| ATOM | 1660 | CA | LEU | 1049 | 20.277 | 13.392 | 38.463 | 1.00 | 29.25 |
| ATOM | 1661 | CB | LEU | 1049 | 21.403 | 12.758 | 39.377 | 1.00 | 32.57 |
| ATOM | 1662 | CG | LEU | 1049 | 22.608 | 12.321 | 38.467 | 1.00 | 36.55 |
| ATOM | 1663 | CD1 | LEU | 1049 | 23.899 | 13.228 | 38.710 | 1.00 | 40.05 |
| ATOM | 1664 | CD2 | LEU | 1049 | 22.814 | 10.823 | 38.652 | 1.00 | 38.04 |
| ATOM | 1665 | C | LEU | 1049 | 19.248 | 14.226 | 39.232 | 1.00 | 29.13 |
| ATOM | 1666 | O | LEU | 1049 | 18.481 | 13.662 | 40.010 | 1.00 | 29.68 |
| ATOM | 1667 | N | ALA | 1050 | 19.267 | 15.581 | 39.111 | 1.00 | 28.60 |
| ATOM | 1668 | H | ALA | 1050 | 20.000 | 15.981 | 38.643 | 1.00 | 25.00 |
| ATOM | 1669 | CA | ALA | 1050 | 18.335 | 16.432 | 39.907 | 1.00 | 27.22 |
| ATOM | 1670 | CB | ALA | 1050 | 18.690 | 17.902 | 39.853 | 1.00 | 23.24 |
| ATOM | 1671 | C | ALA | 1050 | 16.981 | 16.267 | 39.432 | 1.00 | 27.12 |
| ATOM | 1672 | O | ALA | 1050 | 16.073 | 16.803 | 40.054 | 1.00 | 26.89 |
| ATOM | 1673 | N | ARG | 1051 | 16.875 | 15.544 | 38.326 | 1.00 | 27.86 |
| ATOM | 1674 | H | ARG | 1051 | 17.711 | 15.284 | 37.862 | 1.00 | 25.00 |
| ATOM | 1675 | CA | ARG | 1051 | 15.609 | 15.218 | 37.717 | 1.00 | 30.49 |
| ATOM | 1676 | CB | ARG | 1051 | 15.531 | 15.818 | 36.400 | 1.00 | 28.81 |
| ATOM | 1677 | CG | ARG | 1051 | 14.180 | 15.643 | 35.667 | 1.00 | 30.25 |
| ATOM | 1678 | CD | ARG | 1051 | 14.106 | 16.034 | 34.200 | 1.00 | 31.56 |
| ATOM | 1679 | NE | ARG | 1051 | 14.251 | 17.492 | 34.145 | 1.00 | 33.65 |
| ATOM | 1680 | HE | ARG | 1051 | 13.642 | 18.102 | 34.609 | 1.00 | 25.00 |
| ATOM | 1681 | CZ | ARG | 1051 | 15.221 | 18.122 | 33.520 | 1.00 | 35.98 |
| ATOM | 1682 | NH1 | ARG | 1051 | 15.181 | 19.442 | 33.563 | 1.00 | 37.03 |
| ATOM | 1683 | HH11 | ARG | 1051 | 14.444 | 19.896 | 34.053 | 1.00 | 25.00 |
| ATOM | 1684 | HH12 | ARG | 1051 | 15.859 | 19.998 | 33.107 | 1.00 | 25.00 |
| ATOM | 1685 | NH2 | ARG | 1051 | 16.158 | 17.497 | 32.834 | 1.00 | 37.76 |
| ATOM | 1686 | HH21 | ARG | 1051 | 16.176 | 16.505 | 32.764 | 1.00 | 25.00 |
| ATOM | 1687 | HH22 | ARG | 1051 | 16.838 | 18.036 | 32.368 | 1.00 | 25.00 |
| ATOM | 1688 | C | ARG | 1051 | 15.401 | 13.690 | 37.556 | 1.00 | 32.17 |
| ATOM | 1689 | O | ARG | 1051 | 16.235 | 12.967 | 37.015 | 1.00 | 33.05 |
| ATOM | 1690 | N | ASP | 1052 | 14.346 | 13.215 | 38.139 | 1.00 | 33.62 |
| ATOM | 1691 | H | ASP | 1052 | 13.774 | 13.879 | 38.555 | 1.00 | 25.00 |
| ATOM | 1692 | CA | ASP | 1052 | 13.867 | 11.855 | 38.150 | 1.00 | 35.25 |
| ATOM | 1693 | CB | ASP | 1052 | 12.651 | 11.929 | 39.172 | 1.00 | 37.90 |
| ATOM | 1694 | CG | ASP | 1052 | 12.047 | 10.718 | 39.913 | 1.00 | 40.68 |
| ATOM | 1695 | OD1 | ASP | 1052 | 12.404 | 9.596 | 39.596 | 1.00 | 42.47 |
| ATOM | 1696 | OD2 | ASP | 1052 | 11.231 | 10.886 | 40.840 | 1.00 | 38.36 |
| ATOM | 1697 | C | ASP | 1052 | 13.505 | 11.575 | 36.689 | 1.00 | 36.60 |
| ATOM | 1698 | O | ASP | 1052 | 12.330 | 11.710 | 36.313 | 1.00 | 35.79 |
| ATOM | 1699 | N | ILE | 1053 | 14.408 | 11.143 | 35.803 | 1.00 | 38.07 |
| ATOM | 1700 | H | ILE | 1053 | 15.343 | 11.086 | 36.066 | 1.00 | 25.00 |
| ATOM | 1701 | CA | ILE | 1053 | 13.935 | 10.942 | 34.413 | 1.00 | 40.02 |
| ATOM | 1702 | CB | ILE | 1053 | 15.045 | 10.831 | 33.317 | 1.00 | 39.91 |
| ATOM | 1703 | CG2 | ILE | 1053 | 15.741 | 12.188 | 33.301 | 1.00 | 37.74 |
| ATOM | 1704 | CG1 | ILE | 1053 | 15.990 | 9.642 | 33.510 | 1.00 | 39.65 |
| ATOM | 1705 | CD1 | ILE | 1053 | 16.985 | 9.560 | 32.309 | 1.00 | 39.92 |
| ATOM | 1706 | C | ILE | 1053 | 13.059 | 9.724 | 34.222 | 1.00 | 42.46 |
| ATOM | 1707 | O | ILE | 1053 | 12.700 | 9.411 | 33.070 | 1.00 | 41.74 |
| ATOM | 1708 | N | TYR | 1054 | 12.810 | 8.993 | 35.348 | 1.00 | 42.87 |
| ATOM | 1709 | H | TYR | 1054 | 13.210 | 9.205 | 36.201 | 1.00 | 25.00 |
| ATOM | 1710 | CA | TYR | 1054 | 11.890 | 7.886 | 35.169 | 1.00 | 43.17 |
| ATOM | 1711 | CB | TYR | 1054 | 12.344 | 6.532 | 35.787 | 1.00 | 41.56 |
| ATOM | 1712 | CG | TYR | 1054 | 13.619 | 6.142 | 35.147 | 1.00 | 38.81 |
| ATOM | 1713 | CD1 | TYR | 1054 | 14.778 | 6.650 | 35.693 | 1.00 | 40.46 |
| ATOM | 1714 | CE1 | TYR | 1054 | 16.027 | 6.427 | 35.120 | 1.00 | 38.20 |
| ATOM | 1715 | CD2 | TYR | 1054 | 13.754 | 5.385 | 33.985 | 1.00 | 38.65 |
| ATOM | 1716 | CE2 | TYR | 1054 | 15.013 | 5.137 | 33.365 | 1.00 | 34.93 |
| ATOM | 1717 | CZ | TYR | 1054 | 16.128 | 5.678 | 33.966 | 1.00 | 35.95 |
| ATOM | 1718 | OH | TYR | 1054 | 17.371 | 5.536 | 33.428 | 1.00 | 36.73 |
| ATOM | 1719 | HH | TYR | 1054 | 17.311 | 5.066 | 32.602 | 1.00 | 25.00 |
| ATOM | 1720 | C | TYR | 1054 | 10.584 | 8.306 | 35.778 | 1.00 | 44.90 |
| ATOM | 1721 | O | TYR | 1054 | 9.685 | 7.470 | 35.816 | 1.00 | 49.01 |
| ATOM | 1722 | N | LYS | 1055 | 10.393 | 9.506 | 36.363 | 1.00 | 44.39 |
| ATOM | 1723 | H | LYS | 1055 | 11.140 | 10.114 | 36.535 | 1.00 | 25.00 |
| ATOM | 1724 | CA | LYS | 1055 | 8.984 | 9.848 | 36.806 | 1.00 | 44.54 |
| ATOM | 1725 | CB | LYS | 1055 | 8.811 | 10.100 | 38.370 | 1.00 | 45.81 |
| ATOM | 1726 | C | LYS | 1055 | 8.502 | 11.125 | 36.064 | 1.00 | 43.48 |
| ATOM | 1727 | O | LYS | 1055 | 7.501 | 11.763 | 36.420 | 1.00 | 44.33 |
| ATOM | 1728 | N | ASP | 1056 | 9.307 | 11.490 | 35.021 | 1.00 | 41.61 |
| ATOM | 1729 | H | ASP | 1056 | 10.106 | 10.951 | 34.870 | 1.00 | 25.00 |
| ATOM | 1730 | CA | ASP | 1056 | 9.106 | 12.638 | 34.113 | 1.00 | 38.18 |
| ATOM | 1731 | CB | ASP | 1056 | 10.462 | 13.366 | 33.770 | 1.00 | 36.39 |
| ATOM | 1732 | CG | ASP | 1056 | 10.299 | 14.737 | 33.091 | 1.00 | 36.17 |
| ATOM | 1733 | OD1 | ASP | 1056 | 11.232 | 15.296 | 32.625 | 1.00 | 37.70 |
| ATOM | 1734 | OD2 | ASP | 1056 | 9.256 | 15.350 | 33.022 | 1.00 | 34.32 |
| ATOM | 1735 | C | ASP | 1056 | 8.440 | 12.252 | 32.754 | 1.00 | 34.40 |
| ATOM | 1736 | O | ASP | 1056 | 8.929 | 11.447 | 31.955 | 1.00 | 33.38 |
| ATOM | 1737 | N | PRO | 1057 | 7.343 | 12.892 | 32.376 | 1.00 | 30.92 |
| ATOM | 1738 | CD | PRO | 1057 | 6.254 | 13.320 | 33.254 | 1.00 | 27.69 |
| ATOM | 1739 | CA | PRO | 1057 | 6.849 | 12.746 | 31.044 | 1.00 | 27.53 |
| ATOM | 1740 | CB | PRO | 1057 | 5.553 | 13.424 | 30.992 | 1.00 | 25.15 |
| ATOM | 1741 | CG | PRO | 1057 | 5.476 | 14.142 | 32.284 | 1.00 | 26.50 |
| ATOM | 1742 | C | PRO | 1057 | 7.659 | 13.217 | 29.913 | 1.00 | 27.75 |
| ATOM | 1743 | O | PRO | 1057 | 7.126 | 13.155 | 28.821 | 1.00 | 29.79 |
| ATOM | 1744 | N | ASP | 1058 | 8.852 | 13.736 | 29.956 | 1.00 | 27.50 |
| ATOM | 1745 | H | ASP | 1058 | 9.289 | 13.889 | 30.807 | 1.00 | 25.00 |
| ATOM | 1746 | CA | ASP | 1058 | 9.470 | 14.164 | 28.677 | 1.00 | 27.66 |
| ATOM | 1747 | CB | ASP | 1058 | 10.082 | 15.567 | 28.837 | 1.00 | 27.35 |
| ATOM | 1748 | CG | ASP | 1058 | 9.187 | 16.736 | 29.242 | 1.00 | 29.01 |
| ATOM | 1749 | OD1 | ASP | 1058 | 9.778 | 17.741 | 29.638 | 1.00 | 32.75 |
| ATOM | 1750 | OD2 | ASP | 1058 | 7.963 | 16.702 | 29.117 | 1.00 | 25.14 |
| ATOM | 1751 | C | ASP | 1058 | 10.515 | 13.210 | 28.264 | 1.00 | 28.69 |
| ATOM | 1752 | O | ASP | 1058 | 11.298 | 13.361 | 27.300 | 1.00 | 28.44 |
| ATOM | 1753 | N | TYR | 1059 | 10.562 | 12.157 | 29.061 | 1.00 | 31.38 |
| ATOM | 1754 | H | TYR | 1059 | 10.008 | 12.072 | 29.866 | 1.00 | 25.00 |
| ATOM | 1755 | CA | TYR | 1059 | 11.605 | 11.144 | 28.767 | 1.00 | 34.22 |
| ATOM | 1756 | CB | TYR | 1059 | 12.582 | 11.041 | 29.932 | 1.00 | 29.34 |
| ATOM | 1757 | CG | TYR | 1059 | 13.293 | 12.335 | 30.017 | 1.00 | 29.32 |
| ATOM | 1758 | CD1 | TYR | 1059 | 12.773 | 13.393 | 30.820 | 1.00 | 29.65 |
| ATOM | 1759 | CE1 | TYR | 1059 | 13.502 | 14.572 | 31.011 | 1.00 | 19.89 |
| ATOM | 1760 | CD2 | TYR | 1059 | 14.531 | 12.478 | 29.398 | 1.00 | 25.72 |
| ATOM | 1761 | CE2 | TYR | 1059 | 15.234 | 13.655 | 29.614 | 1.00 | 22.55 |
| ATOM | 1762 | CZ | TYR | 1059 | 14.704 | 14.665 | 30.419 | 1.00 | 20.03 |
| ATOM | 1763 | OH | TYR | 1059 | 15.471 | 15.721 | 30.811 | 1.00 | 20.71 |
| ATOM | 1764 | HH | TYR | 1059 | 16.287 | 15.434 | 30.442 | 1.00 | 25.00 |
| ATOM | 1765 | C | TYR | 1059 | 10.838 | 9.892 | 28.609 | 1.00 | 38.30 |
| ATOM | 1766 | O | TYR | 1059 | 10.056 | 9.537 | 29.464 | 1.00 | 38.52 |
| ATOM | 1767 | N | VAL | 1060 | 11.002 | 9.265 | 27.479 | 1.00 | 41.58 |
| ATOM | 1768 | H | VAL | 1060 | 11.610 | 9.681 | 26.842 | 1.00 | 25.00 |
| ATOM | 1769 | CA | VAL | 1060 | 10.318 | 8.044 | 27.089 | 1.00 | 43.40 |
| ATOM | 1770 | CB | VAL | 1060 | 9.623 | 8.577 | 25.832 | 1.00 | 42.99 |
| ATOM | 1771 | CG1 | VAL | 1060 | 9.822 | 7.775 | 24.546 | 1.00 | 44.89 |
| ATOM | 1772 | CG2 | VAL | 1060 | 8.230 | 8.710 | 26.252 | 1.00 | 41.88 |
| ATOM | 1773 | C | VAL | 1060 | 11.276 | 6.810 | 26.950 | 1.00 | 47.40 |
| ATOM | 1774 | O | VAL | 1060 | 12.379 | 6.843 | 26.381 | 1.00 | 45.52 |
| ATOM | 1775 | N | ARG | 1061 | 10.749 | 5.634 | 27.339 | 1.00 | 52.50 |
| ATOM | 1776 | H | ARG | 1061 | 9.802 | 5.632 | 27.586 | 1.00 | 25.00 |
| ATOM | 1777 | CA | ARG | 1061 | 11.486 | 4.356 | 27.363 | 1.00 | 57.10 |
| ATOM | 1778 | CB | ARG | 1061 | 10.738 | 3.309 | 28.224 | 1.00 | 56.78 |
| ATOM | 1779 | C | ARG | 1061 | 11.895 | 3.617 | 26.132 | 1.00 | 60.89 |
| ATOM | 1780 | O | ARG | 1061 | 11.110 | 2.824 | 25.626 | 1.00 | 63.82 |
| ATOM | 1781 | N | LYS | 1062 | 12.956 | 4.010 | 25.463 | 1.00 | 63.22 |
| ATOM | 1782 | H | LYS | 1062 | 13.444 | 4.775 | 25.802 | 1.00 | 25.00 |
| ATOM | 1783 | CA | LYS | 1062 | 13.362 | 3.118 | 24.360 | 1.00 | 67.03 |
| ATOM | 1784 | CB | LYS | 1062 | 14.346 | 3.919 | 23.510 | 1.00 | 65.87 |
| ATOM | 1785 | C | LYS | 1062 | 14.001 | 1.862 | 25.115 | 1.00 | 70.07 |
| ATOM | 1786 | O | LYS | 1062 | 13.400 | 1.379 | 26.094 | 1.00 | 71.13 |
| ATOM | 1787 | N | GLY | 1063 | 15.212 | 1.303 | 24.756 | 1.00 | 73.40 |
| ATOM | 1788 | H | GLY | 1063 | 15.646 | 1.670 | 23.964 | 1.00 | 25.00 |
| ATOM | 1789 | CA | GLY | 1063 | 15.894 | 0.119 | 25.434 | 1.00 | 72.86 |
| ATOM | 1790 | C | GLY | 1063 | 16.242 | 0.366 | 26.911 | 1.00 | 73.67 |
| ATOM | 1791 | O | GLY | 1063 | 15.346 | 0.605 | 27.736 | 1.00 | 74.06 |
| ATOM | 1792 | N | ASP | 1064 | 17.518 | 0.229 | 27.336 | 1.00 | 73.71 |
| ATOM | 1793 | H | ASP | 1064 | 18.219 | −0.126 | 26.769 | 1.00 | 25.00 |
| ATOM | 1794 | CA | ASP | 1064 | 17.802 | 0.547 | 28.763 | 1.00 | 72.28 |
| ATOM | 1795 | CB | ASP | 1064 | 18.899 | −0.416 | 29.317 | 1.00 | 72.63 |
| ATOM | 1796 | C | ASP | 1064 | 18.255 | 2.013 | 28.772 | 1.00 | 70.24 |
| ATOM | 1797 | O | ASP | 1064 | 19.364 | 2.365 | 29.217 | 1.00 | 70.53 |
| ATOM | 1798 | N | ALA | 1065 | 17.344 | 2.836 | 28.147 | 1.00 | 66.71 |
| ATOM | 1799 | H | ALA | 1065 | 16.510 | 2.422 | 27.839 | 1.00 | 25.00 |
| ATOM | 1800 | CA | ALA | 1065 | 17.452 | 4.320 | 27.956 | 1.00 | 62.57 |
| ATOM | 1801 | CB | ALA | 1065 | 18.042 | 4.711 | 26.552 | 1.00 | 61.62 |
| ATOM | 1802 | C | ALA | 1065 | 16.087 | 5.016 | 28.043 | 1.00 | 59.11 |
| ATOM | 1803 | O | ALA | 1065 | 15.176 | 4.509 | 27.388 | 1.00 | 59.09 |
| ATOM | 1804 | N | ARG | 1066 | 15.841 | 5.973 | 28.985 | 1.00 | 54.58 |
| ATOM | 1805 | H | ARG | 1066 | 16.533 | 6.161 | 29.644 | 1.00 | 25.00 |
| ATOM | 1806 | CA | ARG | 1066 | 14.565 | 6.756 | 29.019 | 1.00 | 48.24 |
| ATOM | 1807 | CB | ARG | 1066 | 13.994 | 7.088 | 30.446 | 1.00 | 45.40 |
| ATOM | 1808 | C | ARG | 1066 | 15.023 | 8.085 | 28.365 | 1.00 | 45.68 |
| ATOM | 1809 | O | ARG | 1066 | 15.715 | 8.908 | 28.958 | 1.00 | 45.21 |
| ATOM | 1810 | N | LEU | 1067 | 14.851 | 8.192 | 27.051 | 1.00 | 42.30 |
| ATOM | 1811 | H | LEU | 1067 | 14.419 | 7.435 | 26.603 | 1.00 | 25.00 |
| ATOM | 1812 | CA | LEU | 1067 | 15.198 | 9.345 | 26.231 | 1.00 | 37.69 |
| ATOM | 1813 | CB | LEU | 1067 | 15.531 | 8.838 | 24.899 | 1.00 | 36.61 |
| ATOM | 1814 | CG | LEU | 1067 | 16.690 | 7.827 | 24.982 | 1.00 | 34.60 |
| ATOM | 1815 | CD1 | LEU | 1067 | 17.019 | 7.123 | 23.699 | 1.00 | 33.02 |
| ATOM | 1816 | CD2 | LEU | 1067 | 17.919 | 8.551 | 25.007 | 1.00 | 34.41 |
| ATOM | 1817 | C | LEU | 1067 | 14.222 | 10.500 | 26.047 | 1.00 | 37.46 |
| ATOM | 1818 | O | LEU | 1067 | 13.042 | 10.305 | 25.809 | 1.00 | 33.35 |
| ATOM | 1819 | N | PRO | 1068 | 14.730 | 11.771 | 25.971 | 1.00 | 40.30 |
| ATOM | 1820 | CD | PRO | 1068 | 16.149 | 12.168 | 26.330 | 1.00 | 39.73 |
| ATOM | 1821 | CA | PRO | 1068 | 13.885 | 13.014 | 25.586 | 1.00 | 38.91 |
| ATOM | 1822 | CB | PRO | 1068 | 14.846 | 14.211 | 25.936 | 1.00 | 39.54 |
| ATOM | 1823 | CG | PRO | 1068 | 16.273 | 13.592 | 25.775 | 1.00 | 39.14 |
| ATOM | 1824 | C | PRO | 1068 | 13.408 | 12.992 | 24.114 | 1.00 | 36.97 |
| ATOM | 1825 | O | PRO | 1068 | 13.701 | 13.817 | 23.228 | 1.00 | 32.57 |
| ATOM | 1826 | N | LEU | 1069 | 12.612 | 11.969 | 23.782 | 1.00 | 36.65 |
| ATOM | 1827 | H | LEU | 1069 | 12.330 | 11.363 | 24.480 | 1.00 | 25.00 |
| ATOM | 1828 | CA | LEU | 1069 | 12.221 | 11.789 | 22.344 | 1.00 | 34.21 |
| ATOM | 1829 | CB | LEU | 1069 | 11.251 | 10.560 | 22.261 | 1.00 | 34.68 |
| ATOM | 1830 | CG | LEU | 1069 | 11.920 | 9.187 | 21.857 | 1.00 | 35.25 |
| ATOM | 1831 | CD1 | LEU | 1069 | 13.094 | 9.514 | 20.844 | 1.00 | 34.09 |
| ATOM | 1832 | CD2 | LEU | 1069 | 12.622 | 8.441 | 23.030 | 1.00 | 36.06 |
| ATOM | 1833 | C | LEU | 1069 | 11.660 | 12.996 | 21.621 | 1.00 | 27.83 |
| ATOM | 1834 | O | LEU | 1069 | 12.220 | 13.404 | 20.649 | 1.00 | 24.23 |
| ATOM | 1835 | N | LYS | 1070 | 10.586 | 13.605 | 22.014 | 1.00 | 24.00 |
| ATOM | 1836 | H | LYS | 1070 | 10.036 | 13.333 | 22.730 | 1.00 | 25.00 |
| ATOM | 1837 | CA | LYS | 1070 | 9.967 | 14.754 | 21.451 | 1.00 | 22.99 |
| ATOM | 1838 | CB | LYS | 1070 | 9.049 | 15.201 | 22.506 | 1.00 | 20.18 |
| ATOM | 1839 | CG | LYS | 1070 | 7.725 | 14.545 | 22.406 | 1.00 | 16.93 |
| ATOM | 1840 | CD | LYS | 1070 | 6.602 | 15.201 | 23.201 | 1.00 | 19.52 |
| ATOM | 1841 | CE | LYS | 1070 | 6.712 | 15.321 | 24.701 | 1.00 | 22.27 |
| ATOM | 1842 | NZ | LYS | 1070 | 5.318 | 15.611 | 25.105 | 1.00 | 23.89 |
| ATOM | 1843 | HZ1 | LYS | 1070 | 4.715 | 14.927 | 24.606 | 1.00 | 25.00 |
| ATOM | 1844 | HZ2 | LYS | 1070 | 5.086 | 16.585 | 24.825 | 1.00 | 25.00 |
| ATOM | 1845 | HZ3 | LYS | 1070 | 5.235 | 15.514 | 26.143 | 1.00 | 25.00 |
| ATOM | 1846 | C | LYS | 1070 | 11.001 | 15.784 | 21.078 | 1.00 | 27.06 |
| ATOM | 1847 | O | LYS | 1070 | 10.942 | 16.561 | 20.101 | 1.00 | 29.34 |
| ATOM | 1848 | N | TRP | 1071 | 12.081 | 15.771 | 21.805 | 1.00 | 28.16 |
| ATOM | 1849 | H | TRP | 1071 | 12.226 | 15.082 | 22.472 | 1.00 | 25.00 |
| ATOM | 1850 | CA | TRP | 1071 | 13.144 | 16.742 | 21.493 | 1.00 | 29.14 |
| ATOM | 1851 | CB | TRP | 1071 | 13.828 | 17.238 | 22.761 | 1.00 | 24.34 |
| ATOM | 1852 | CG | TRP | 1071 | 12.988 | 18.047 | 23.581 | 1.00 | 21.47 |
| ATOM | 1853 | CD2 | TRP | 1071 | 11.839 | 17.658 | 24.311 | 1.00 | 20.60 |
| ATOM | 1854 | CE2 | TRP | 1071 | 11.261 | 18.844 | 24.807 | 1.00 | 18.15 |
| ATOM | 1855 | CE3 | TRP | 1071 | 11.235 | 16.432 | 24.586 | 1.00 | 21.35 |
| ATOM | 1856 | CD1 | TRP | 1071 | 13.097 | 19.440 | 23.663 | 1.00 | 21.95 |
| ATOM | 1857 | NE1 | TRP | 1071 | 12.047 | 19.888 | 24.396 | 1.00 | 21.56 |
| ATOM | 1858 | HE1 | TRP | 1071 | 11.851 | 20.814 | 24.639 | 1.00 | 25.00 |
| ATOM | 1859 | CZ2 | TRP | 1071 | 10.100 | 18.794 | 25.565 | 1.00 | 15.70 |
| ATOM | 1860 | CZ3 | TRP | 1071 | 10.042 | 16.373 | 25.332 | 1.00 | 21.07 |
| ATOM | 1861 | CH2 | TRP | 1071 | 9.503 | 17.563 | 25.830 | 1.00 | 21.22 |
| ATOM | 1862 | C | TRP | 1071 | 14.295 | 16.314 | 20.599 | 1.00 | 30.80 |
| ATOM | 1863 | O | TRP | 1071 | 15.079 | 17.247 | 20.253 | 1.00 | 31.15 |
| ATOM | 1864 | N | MET | 1072 | 14.457 | 15.001 | 20.219 | 1.00 | 31.49 |
| ATOM | 1865 | H | MET | 1072 | 13.767 | 14.348 | 20.421 | 1.00 | 25.00 |
| ATOM | 1866 | CA | MET | 1072 | 15.723 | 14.596 | 19.467 | 1.00 | 32.07 |
| ATOM | 1867 | CB | MET | 1072 | 16.272 | 13.142 | 20.012 | 1.00 | 34.06 |
| ATOM | 1868 | CG | MET | 1072 | 15.745 | 12.668 | 21.398 | 1.00 | 36.30 |
| ATOM | 1869 | SD | MET | 1072 | 16.353 | 11.109 | 22.089 | 1.00 | 36.15 |
| ATOM | 1870 | CE | MET | 1072 | 17.895 | 11.697 | 22.788 | 1.00 | 40.32 |
| ATOM | 1871 | C | MET | 1072 | 15.759 | 14.596 | 17.968 | 1.00 | 31.33 |
| ATOM | 1872 | O | MET | 1072 | 14.705 | 14.400 | 17.363 | 1.00 | 29.91 |
| ATOM | 1873 | N | ALA | 1073 | 16.939 | 14.772 | 17.362 | 1.00 | 30.05 |
| ATOM | 1874 | H | ALA | 1073 | 17.765 | 14.776 | 17.893 | 1.00 | 25.00 |
| ATOM | 1875 | CA | ALA | 1073 | 16.879 | 14.778 | 15.909 | 1.00 | 33.91 |
| ATOM | 1876 | CB | ALA | 1073 | 18.155 | 15.325 | 15.376 | 1.00 | 32.06 |
| ATOM | 1877 | C | ALA | 1073 | 16.675 | 13.357 | 15.318 | 1.00 | 39.04 |
| ATOM | 1878 | O | ALA | 1073 | 17.347 | 12.372 | 15.714 | 1.00 | 40.39 |
| ATOM | 1879 | N | PRO | 1074 | 15.818 | 13.167 | 14.325 | 1.00 | 40.79 |
| ATOM | 1880 | CD | PRO | 1074 | 14.403 | 13.372 | 14.228 | 1.00 | 41.14 |
| ATOM | 1881 | CA | PRO | 1074 | 16.002 | 12.117 | 13.375 | 1.00 | 43.11 |
| ATOM | 1882 | CB | PRO | 1074 | 15.642 | 12.879 | 12.142 | 1.00 | 41.14 |
| ATOM | 1883 | CG | PRO | 1074 | 14.268 | 12.831 | 12.804 | 1.00 | 42.64 |
| ATOM | 1884 | C | PRO | 1074 | 17.293 | 11.334 | 13.387 | 1.00 | 45.76 |
| ATOM | 1885 | O | PRO | 1074 | 17.140 | 10.195 | 13.761 | 1.00 | 47.78 |
| ATOM | 1886 | N | GLU | 1075 | 18.548 | 11.750 | 13.155 | 1.00 | 46.56 |
| ATOM | 1887 | H | GLU | 1075 | 18.675 | 12.692 | 13.021 | 1.00 | 25.00 |
| ATOM | 1888 | CA | GLU | 1075 | 19.636 | 10.744 | 13.200 | 1.00 | 45.95 |
| ATOM | 1889 | CB | GLU | 1075 | 20.810 | 11.312 | 12.402 | 1.00 | 44.97 |
| ATOM | 1890 | CG | GLU | 1075 | 21.733 | 12.301 | 13.044 | 1.00 | 44.30 |
| ATOM | 1891 | CD | GLU | 1075 | 21.159 | 13.596 | 13.527 | 1.00 | 40.87 |
| ATOM | 1892 | OE1 | GLU | 1075 | 20.068 | 13.949 | 13.066 | 1.00 | 32.82 |
| ATOM | 1893 | OE2 | GLU | 1075 | 21.882 | 14.238 | 14.325 | 1.00 | 41.53 |
| ATOM | 1894 | C | GLU | 1075 | 20.061 | 10.240 | 14.598 | 1.00 | 48.08 |
| ATOM | 1895 | O | GLU | 1075 | 20.640 | 9.160 | 14.770 | 1.00 | 49.35 |
| ATOM | 1896 | N | THR | 1076 | 19.865 | 11.016 | 15.669 | 1.00 | 49.29 |
| ATOM | 1897 | H | THR | 1076 | 19.524 | 11.922 | 15.554 | 1.00 | 25.00 |
| ATOM | 1898 | CA | THR | 1076 | 20.185 | 10.516 | 17.025 | 1.00 | 48.50 |
| ATOM | 1899 | CB | THR | 1076 | 19.962 | 11.565 | 18.093 | 1.00 | 47.69 |
| ATOM | 1900 | OG1 | THR | 1076 | 20.569 | 12.790 | 17.681 | 1.00 | 45.05 |
| ATOM | 1901 | HG1 | THR | 1076 | 20.446 | 13.406 | 18.403 | 1.00 | 25.00 |
| ATOM | 1902 | CG2 | THR | 1076 | 20.477 | 11.019 | 19.433 | 1.00 | 45.23 |
| ATOM | 1903 | C | THR | 1076 | 19.157 | 9.411 | 17.252 | 1.00 | 47.90 |
| ATOM | 1904 | O | THR | 1076 | 19.575 | 8.274 | 17.334 | 1.00 | 49.34 |
| ATOM | 1905 | N | ILE | 1077 | 17.827 | 9.672 | 17.313 | 1.00 | 48.47 |
| ATOM | 1906 | H | ILE | 1077 | 17.502 | 10.597 | 17.256 | 1.00 | 25.00 |
| ATOM | 1907 | CA | ILE | 1077 | 16.792 | 8.590 | 17.487 | 1.00 | 49.42 |
| ATOM | 1908 | CB | ILE | 1077 | 15.425 | 9.178 | 16.842 | 1.00 | 44.15 |
| ATOM | 1909 | CG2 | ILE | 1077 | 14.319 | 8.109 | 16.703 | 1.00 | 44.75 |
| ATOM | 1910 | CG1 | ILE | 1077 | 14.756 | 10.190 | 17.788 | 1.00 | 37.92 |
| ATOM | 1911 | CD1 | ILE | 1077 | 13.272 | 10.626 | 17.422 | 1.00 | 27.84 |
| ATOM | 1912 | C | ILE | 1077 | 17.243 | 7.175 | 16.861 | 1.00 | 53.36 |
| ATOM | 1913 | O | ILE | 1077 | 17.291 | 6.072 | 17.461 | 1.00 | 54.57 |
| ATOM | 1914 | N | PHE | 1078 | 17.701 | 7.255 | 15.614 | 1.00 | 55.06 |
| ATOM | 1915 | H | PHE | 1078 | 17.740 | 8.118 | 15.170 | 1.00 | 25.00 |
| ATOM | 1916 | CA | PHE | 1078 | 18.167 | 6.084 | 14.914 | 1.00 | 56.25 |
| ATOM | 1917 | CB | PHE | 1078 | 17.885 | 6.390 | 13.423 | 1.00 | 56.31 |
| ATOM | 1918 | CG | PHE | 1078 | 16.371 | 6.525 | 13.301 | 1.00 | 58.21 |
| ATOM | 1919 | CD1 | PHE | 1078 | 15.751 | 7.753 | 13.589 | 1.00 | 57.13 |
| ATOM | 1920 | CD2 | PHE | 1078 | 15.524 | 5.456 | 12.917 | 1.00 | 58.52 |
| ATOM | 1921 | CE1 | PHE | 1078 | 14.374 | 7.914 | 13.501 | 1.00 | 55.45 |
| ATOM | 1922 | CE2 | PHE | 1078 | 14.128 | 5.599 | 12.821 | 1.00 | 56.84 |
| ATOM | 1923 | CZ | PHE | 1078 | 13.571 | 6.837 | 13.119 | 1.00 | 57.27 |
| ATOM | 1924 | C | PHE | 1078 | 19.610 | 5.696 | 15.228 | 1.00 | 57.28 |
| ATOM | 1925 | O | PHE | 1078 | 19.902 | 4.786 | 16.028 | 1.00 | 58.40 |
| ATOM | 1926 | N | ASP | 1079 | 20.584 | 6.376 | 14.678 | 1.00 | 57.15 |
| ATOM | 1927 | H | ASP | 1079 | 20.483 | 7.203 | 14.172 | 1.00 | 25.00 |
| ATOM | 1928 | CA | ASP | 1079 | 21.895 | 5.888 | 15.009 | 1.00 | 58.15 |
| ATOM | 1929 | CB | ASP | 1079 | 22.609 | 5.621 | 13.634 | 1.00 | 59.05 |
| ATOM | 1930 | CG | ASP | 1079 | 22.069 | 6.328 | 12.354 | 1.00 | 61.20 |
| ATOM | 1931 | OD1 | ASP | 1079 | 20.858 | 6.288 | 12.027 | 1.00 | 58.47 |
| ATOM | 1932 | OD2 | ASP | 1079 | 22.932 | 6.900 | 11.669 | 1.00 | 62.91 |
| ATOM | 1933 | C | ASP | 1079 | 22.666 | 6.733 | 16.013 | 1.00 | 59.81 |
| ATOM | 1934 | O | ASP | 1079 | 23.784 | 7.236 | 15.792 | 1.00 | 61.48 |
| ATOM | 1935 | N | ARG | 1080 | 22.033 | 6.843 | 17.207 | 1.00 | 59.87 |
| ATOM | 1936 | H | ARG | 1080 | 21.133 | 6.436 | 17.251 | 1.00 | 25.00 |
| ATOM | 1937 | CA | ARG | 1080 | 22.560 | 7.566 | 18.422 | 1.00 | 59.31 |
| ATOM | 1938 | CB | ARG | 1080 | 22.684 | 6.469 | 19.508 | 1.00 | 65.88 |
| ATOM | 1939 | CG | ARG | 1080 | 21.458 | 5.516 | 19.732 | 1.00 | 72.03 |
| ATOM | 1940 | CD | ARG | 1080 | 22.045 | 4.310 | 20.564 | 1.00 | 79.90 |
| ATOM | 1941 | NE | ARG | 1080 | 22.920 | 3.385 | 19.754 | 1.00 | 87.63 |
| ATOM | 1942 | HE | ARG | 1080 | 22.968 | 3.578 | 18.793 | 1.00 | 25.00 |
| ATOM | 1943 | CZ | ARG | 1080 | 23.672 | 2.343 | 20.253 | 1.00 | 90.99 |
| ATOM | 1944 | NH1 | ARG | 1080 | 24.402 | 1.565 | 19.417 | 1.00 | 92.40 |
| ATOM | 1945 | HH11 | ARG | 1080 | 24.402 | 1.756 | 18.435 | 1.00 | 25.00 |
| ATOM | 1946 | HH12 | ARG | 1080 | 24.916 | 0.797 | 19.786 | 1.00 | 25.00 |
| ATOM | 1947 | NH2 | ARG | 1080 | 23.686 | 2.054 | 21.571 | 1.00 | 91.90 |
| ATOM | 1948 | HH21 | ARG | 1080 | 23.138 | 2.606 | 22.195 | 1.00 | 25.00 |
| ATOM | 1949 | HH22 | ARG | 1080 | 24.207 | 1.275 | 21.903 | 1.00 | 25.00 |
| ATOM | 1950 | C | ARG | 1080 | 23.866 | 8.459 | 18.385 | 1.00 | 55.82 |
| ATOM | 1951 | O | ARG | 1080 | 24.807 | 8.324 | 19.178 | 1.00 | 54.55 |
| ATOM | 1952 | N | VAL | 1081 | 23.865 | 9.360 | 17.393 | 1.00 | 52.40 |
| ATOM | 1953 | H | VAL | 1081 | 23.074 | 9.231 | 16.833 | 1.00 | 25.00 |
| ATOM | 1954 | CA | VAL | 1081 | 24.812 | 10.458 | 16.972 | 1.00 | 48.84 |
| ATOM | 1955 | CB | VAL | 1081 | 24.778 | 10.791 | 15.356 | 1.00 | 49.94 |
| ATOM | 1956 | CG1 | VAL | 1081 | 24.407 | 12.238 | 14.969 | 1.00 | 49.32 |
| ATOM | 1957 | CG2 | VAL | 1081 | 26.151 | 10.924 | 14.772 | 1.00 | 48.88 |
| ATOM | 1958 | C | VAL | 1081 | 24.233 | 11.708 | 17.675 | 1.00 | 46.62 |
| ATOM | 1959 | O | VAL | 1081 | 23.049 | 12.091 | 17.516 | 1.00 | 46.67 |
| ATOM | 1960 | N | TYR | 1082 | 25.063 | 12.319 | 18.473 | 1.00 | 42.76 |
| ATOM | 1961 | H | TYR | 1082 | 25.938 | 11.901 | 18.573 | 1.00 | 25.00 |
| ATOM | 1962 | CA | TYR | 1082 | 24.740 | 13.562 | 19.170 | 1.00 | 39.04 |
| ATOM | 1963 | CB | TYR | 1082 | 24.932 | 13.403 | 20.674 | 1.00 | 38.16 |
| ATOM | 1964 | CG | TYR | 1082 | 23.827 | 12.900 | 21.446 | 1.00 | 34.58 |
| ATOM | 1965 | CD1 | TYR | 1082 | 24.056 | 11.769 | 22.211 | 1.00 | 38.58 |
| ATOM | 1966 | CE1 | TYR | 1082 | 22.986 | 11.240 | 22.964 | 1.00 | 37.53 |
| ATOM | 1967 | CD2 | TYR | 1082 | 22.615 | 13.512 | 21.416 | 1.00 | 33.99 |
| ATOM | 1968 | CE2 | TYR | 1082 | 21.543 | 12.997 | 22.158 | 1.00 | 36.25 |
| ATOM | 1969 | CZ | TYR | 1082 | 21.714 | 11.845 | 22.934 | 1.00 | 39.08 |
| ATOM | 1970 | OH | TYR | 1082 | 20.620 | 11.198 | 23.518 | 1.00 | 41.35 |
| ATOM | 1971 | HH | TYR | 1082 | 20.981 | 10.409 | 23.945 | 1.00 | 25.00 |
| ATOM | 1972 | C | TYR | 1082 | 25.804 | 14.518 | 18.605 | 1.00 | 38.97 |
| ATOM | 1973 | O | TYR | 1082 | 27.003 | 14.214 | 18.715 | 1.00 | 40.63 |
| ATOM | 1974 | N | THR | 1083 | 25.415 | 15.547 | 17.865 | 1.00 | 35.41 |
| ATOM | 1975 | H | THR | 1083 | 24.467 | 15.654 | 17.662 | 1.00 | 25.00 |
| ATOM | 1976 | CA | THR | 1083 | 26.367 | 16.508 | 17.300 | 1.00 | 35.10 |
| ATOM | 1977 | CB | THR | 1083 | 26.416 | 16.542 | 15.819 | 1.00 | 32.77 |
| ATOM | 1978 | OG1 | THR | 1083 | 25.109 | 16.731 | 15.306 | 1.00 | 32.46 |
| ATOM | 1979 | HG1 | THR | 1083 | 25.105 | 16.789 | 14.345 | 1.00 | 25.00 |
| ATOM | 1980 | CG2 | THR | 1083 | 26.996 | 15.278 | 15.338 | 1.00 | 33.29 |
| ATOM | 1981 | C | THR | 1083 | 25.927 | 17.902 | 17.642 | 1.00 | 38.20 |
| ATOM | 1982 | O | THR | 1083 | 24.767 | 17.983 | 18.081 | 1.00 | 38.44 |
| ATOM | 1983 | N | ILE | 1084 | 26.693 | 19.054 | 17.528 | 1.00 | 38.84 |
| ATOM | 1984 | H | ILE | 1084 | 27.652 | 19.050 | 17.358 | 1.00 | 25.00 |
| ATOM | 1985 | CA | ILE | 1084 | 25.899 | 20.273 | 17.913 | 1.00 | 39.23 |
| ATOM | 1986 | CB | ILE | 1084 | 26.593 | 21.712 | 17.946 | 1.00 | 42.16 |
| ATOM | 1987 | CG2 | ILE | 1084 | 27.085 | 21.957 | 19.405 | 1.00 | 44.04 |
| ATOM | 1988 | CG1 | ILE | 1084 | 27.745 | 21.882 | 16.950 | 1.00 | 44.94 |
| ATOM | 1989 | CD1 | ILE | 1084 | 27.347 | 22.047 | 15.465 | 1.00 | 47.97 |
| ATOM | 1990 | C | ILE | 1084 | 24.752 | 20.429 | 16.928 | 1.00 | 37.38 |
| ATOM | 1991 | O | ILE | 1084 | 23.780 | 21.127 | 17.149 | 1.00 | 37.85 |
| ATOM | 1992 | N | GLN | 1085 | 24.741 | 19.787 | 15.783 | 1.00 | 36.36 |
| ATOM | 1993 | H | GLN | 1085 | 25.511 | 19.263 | 15.506 | 1.00 | 25.00 |
| ATOM | 1994 | CA | GLN | 1085 | 23.591 | 19.988 | 14.905 | 1.00 | 32.80 |
| ATOM | 1995 | CB | GLN | 1085 | 23.952 | 19.645 | 13.512 | 1.00 | 38.87 |
| ATOM | 1996 | CG | GLN | 1085 | 25.018 | 20.710 | 12.997 | 1.00 | 48.38 |
| ATOM | 1997 | CD | GLN | 1085 | 24.633 | 22.276 | 13.040 | 1.00 | 51.52 |
| ATOM | 1998 | OE1 | GLN | 1085 | 25.315 | 23.141 | 13.617 | 1.00 | 55.17 |
| ATOM | 1999 | NE2 | GLN | 1085 | 23.575 | 22.749 | 12.400 | 1.00 | 49.91 |
| ATOM | 2000 | HE21 | GLN | 1085 | 22.997 | 22.158 | 11.887 | 1.00 | 25.00 |
| ATOM | 2001 | HE22 | GLN | 1085 | 23.399 | 23.705 | 12.459 | 1.00 | 25.00 |
| ATOM | 2002 | C | GLN | 1085 | 22.460 | 19.252 | 15.371 | 1.00 | 29.29 |
| ATOM | 2003 | O | GLN | 1085 | 21.365 | 19.542 | 14.958 | 1.00 | 29.41 |
| ATOM | 2004 | N | SER | 1086 | 22.648 | 18.247 | 16.203 | 1.00 | 27.89 |
| ATOM | 2005 | H | SER | 1086 | 23.528 | 17.831 | 16.357 | 1.00 | 25.00 |
| ATOM | 2006 | CA | SER | 1086 | 21.420 | 17.595 | 16.735 | 1.00 | 27.39 |
| ATOM | 2007 | CB | SER | 1086 | 21.688 | 16.215 | 17.368 | 1.00 | 26.64 |
| ATOM | 2008 | OG | SER | 1086 | 22.800 | 16.196 | 18.229 | 1.00 | 32.26 |
| ATOM | 2009 | HG | SER | 1086 | 22.880 | 15.316 | 18.608 | 1.00 | 25.00 |
| ATOM | 2010 | C | SER | 1086 | 20.906 | 18.552 | 17.796 | 1.00 | 28.52 |
| ATOM | 2011 | O | SER | 1086 | 19.689 | 18.681 | 18.089 | 1.00 | 28.65 |
| ATOM | 2012 | N | ASP | 1087 | 21.891 | 19.284 | 18.414 | 1.00 | 28.74 |
| ATOM | 2013 | H | ASP | 1087 | 22.823 | 19.181 | 18.119 | 1.00 | 25.00 |
| ATOM | 2014 | CA | ASP | 1087 | 21.539 | 20.382 | 19.427 | 1.00 | 28.48 |
| ATOM | 2015 | CB | ASP | 1087 | 22.810 | 20.997 | 20.094 | 1.00 | 28.41 |
| ATOM | 2016 | CG | ASP | 1087 | 23.416 | 20.294 | 21.317 | 1.00 | 25.14 |
| ATOM | 2017 | OD1 | ASP | 1087 | 24.561 | 20.580 | 21.642 | 1.00 | 21.22 |
| ATOM | 2018 | OD2 | ASP | 1087 | 22.713 | 19.538 | 21.992 | 1.00 | 23.35 |
| ATOM | 2019 | C | ASP | 1087 | 20.726 | 21.545 | 18.743 | 1.00 | 26.87 |
| ATOM | 2020 | O | ASP | 1087 | 19.712 | 22.040 | 19.291 | 1.00 | 26.46 |
| ATOM | 2021 | N | VAL | 1088 | 21.063 | 21.872 | 17.455 | 1.00 | 26.89 |
| ATOM | 2022 | H | VAL | 1088 | 21.834 | 21.448 | 17.002 | 1.00 | 25.00 |
| ATOM | 2023 | CA | VAL | 1088 | 20.275 | 22.889 | 16.739 | 1.00 | 24.93 |
| ATOM | 2024 | CB | VAL | 1088 | 20.842 | 23.077 | 15.389 | 1.00 | 23.65 |
| ATOM | 2025 | CG1 | VAL | 1088 | 19.903 | 24.065 | 14.729 | 1.00 | 29.19 |
| ATOM | 2026 | CG2 | VAL | 1088 | 22.301 | 23.476 | 15.401 | 1.00 | 21.70 |
| ATOM | 2027 | C | VAL | 1088 | 18.863 | 22.326 | 16.670 | 1.00 | 26.76 |
| ATOM | 2028 | O | VAL | 1088 | 17.944 | 22.953 | 17.146 | 1.00 | 28.15 |
| ATOM | 2029 | N | TRP | 1089 | 18.607 | 21.051 | 16.252 | 1.00 | 29.94 |
| ATOM | 2030 | H | TRP | 1089 | 19.349 | 20.557 | 15.855 | 1.00 | 25.00 |
| ATOM | 2031 | CA | TRP | 1089 | 17.221 | 20.447 | 16.142 | 1.00 | 26.99 |
| ATOM | 2032 | CB | TRP | 1089 | 17.308 | 18.833 | 15.816 | 1.00 | 23.39 |
| ATOM | 2033 | CG | TRP | 1089 | 15.971 | 18.154 | 15.749 | 1.00 | 20.14 |
| ATOM | 2034 | CD2 | TRP | 1089 | 15.248 | 17.719 | 14.606 | 1.00 | 19.39 |
| ATOM | 2035 | CE2 | TRP | 1089 | 13.948 | 17.436 | 14.988 | 1.00 | 21.49 |
| ATOM | 2036 | CE3 | TRP | 1089 | 15.570 | 17.556 | 13.259 | 1.00 | 22.51 |
| ATOM | 2037 | CD1 | TRP | 1089 | 15.026 | 18.102 | 16.799 | 1.00 | 20.76 |
| ATOM | 2038 | NE1 | TRP | 1089 | 13.796 | 17.671 | 16.377 | 1.00 | 22.04 |
| ATOM | 2039 | HE1 | TRP | 1089 | 12.988 | 17.520 | 16.926 | 1.00 | 25.00 |
| ATOM | 2040 | CZ2 | TRP | 1089 | 13.043 | 17.012 | 13.997 | 1.00 | 19.47 |
| ATOM | 2041 | CZ3 | TRP | 1089 | 14.699 | 17.133 | 12.279 | 1.00 | 19.86 |
| ATOM | 2042 | CH2 | TRP | 1089 | 13.414 | 16.865 | 12.672 | 1.00 | 23.72 |
| ATOM | 2043 | C | TRP | 1089 | 16.454 | 20.678 | 17.423 | 1.00 | 26.12 |
| ATOM | 2044 | O | TRP | 1089 | 15.295 | 21.133 | 17.448 | 1.00 | 28.13 |
| ATOM | 2045 | N | SER | 1090 | 17.040 | 20.297 | 18.553 | 1.00 | 25.26 |
| ATOM | 2046 | H | SER | 1090 | 17.937 | 19.919 | 18.539 | 1.00 | 25.00 |
| ATOM | 2047 | CA | SER | 1090 | 16.239 | 20.524 | 19.757 | 1.00 | 23.03 |
| ATOM | 2048 | CB | SER | 1090 | 16.712 | 19.652 | 20.987 | 1.00 | 20.72 |
| ATOM | 2049 | OG | SER | 1090 | 18.066 | 19.147 | 20.915 | 1.00 | 19.68 |
| ATOM | 2050 | HG | SER | 1090 | 18.072 | 18.373 | 21.471 | 1.00 | 25.00 |
| ATOM | 2051 | C | SER | 1090 | 16.199 | 22.034 | 20.090 | 1.00 | 22.43 |
| ATOM | 2052 | O | SER | 1090 | 15.228 | 22.381 | 20.782 | 1.00 | 21.00 |
| ATOM | 2053 | N | PHE | 1091 | 17.201 | 22.904 | 19.726 | 1.00 | 21.18 |
| ATOM | 2054 | H | PHE | 1091 | 17.980 | 22.546 | 19.235 | 1.00 | 25.00 |
| ATOM | 2055 | CA | PHE | 1091 | 17.050 | 24.381 | 19.885 | 1.00 | 22.59 |
| ATOM | 2056 | CB | PHE | 1091 | 18.195 | 25.104 | 19.230 | 1.00 | 28.95 |
| ATOM | 2057 | CG | PHE | 1091 | 18.027 | 26.570 | 19.483 | 1.00 | 36.08 |
| ATOM | 2058 | CD1 | PHE | 1091 | 18.027 | 27.065 | 20.826 | 1.00 | 36.59 |
| ATOM | 2059 | CD2 | PHE | 1091 | 17.797 | 27.474 | 18.397 | 1.00 | 36.98 |
| ATOM | 2060 | CE1 | PHE | 1091 | 17.792 | 28.427 | 21.082 | 1.00 | 34.62 |
| ATOM | 2061 | CE2 | PHE | 1091 | 17.564 | 28.838 | 18.654 | 1.00 | 36.85 |
| ATOM | 2062 | CZ | PHE | 1091 | 17.557 | 29.279 | 20.000 | 1.00 | 36.80 |
| ATOM | 2063 | C | PHE | 1091 | 15.773 | 24.706 | 19.112 | 1.00 | 20.11 |
| ATOM | 2064 | O | PHE | 1091 | 14.804 | 25.357 | 19.549 | 1.00 | 20.48 |
| ATOM | 2065 | N | GLY | 1092 | 15.569 | 24.187 | 17.936 | 1.00 | 20.58 |
| ATOM | 2066 | H | GLY | 1092 | 16.257 | 23.584 | 17.562 | 1.00 | 25.00 |
| ATOM | 2067 | CA | GLY | 1092 | 14.244 | 24.379 | 17.158 | 1.00 | 20.51 |
| ATOM | 2068 | C | GLY | 1092 | 13.042 | 23.954 | 17.976 | 1.00 | 21.35 |
| ATOM | 2069 | O | GLY | 1092 | 12.025 | 24.637 | 18.077 | 1.00 | 25.27 |
| ATOM | 2070 | N | VAL | 1093 | 13.115 | 22.852 | 18.740 | 1.00 | 22.05 |
| ATOM | 2071 | H | VAL | 1093 | 13.958 | 22.330 | 18.789 | 1.00 | 25.00 |
| ATOM | 2072 | CA | VAL | 1093 | 11.925 | 22.484 | 19.543 | 1.00 | 21.47 |
| ATOM | 2073 | CB | VAL | 1093 | 12.168 | 21.022 | 20.058 | 1.00 | 19.28 |
| ATOM | 2074 | CG1 | VAL | 1093 | 10.977 | 20.627 | 20.845 | 1.00 | 17.99 |
| ATOM | 2075 | CG2 | VAL | 1093 | 12.433 | 20.009 | 18.948 | 1.00 | 17.10 |
| ATOM | 2076 | C | VAL | 1093 | 11.761 | 23.541 | 20.694 | 1.00 | 23.39 |
| ATOM | 2077 | O | VAL | 1093 | 10.667 | 23.822 | 21.227 | 1.00 | 26.02 |
| ATOM | 2078 | N | LEU | 1094 | 12.876 | 24.030 | 21.303 | 1.00 | 24.64 |
| ATOM | 2079 | H | LEU | 1094 | 13.734 | 23.682 | 20.979 | 1.00 | 25.00 |
| ATOM | 2080 | CA | LEU | 1094 | 12.930 | 25.109 | 22.384 | 1.00 | 21.12 |
| ATOM | 2081 | CB | LEU | 1094 | 14.350 | 25.628 | 22.624 | 1.00 | 21.26 |
| ATOM | 2082 | CG | LEU | 1094 | 14.855 | 25.817 | 24.033 | 1.00 | 19.99 |
| ATOM | 2083 | CD1 | LEU | 1094 | 15.842 | 26.870 | 23.939 | 1.00 | 16.60 |
| ATOM | 2084 | CD2 | LEU | 1094 | 13.846 | 26.320 | 24.994 | 1.00 | 19.25 |
| ATOM | 2085 | C | LEU | 1094 | 12.189 | 26.302 | 21.875 | 1.00 | 20.18 |
| ATOM | 2086 | O | LEU | 1094 | 11.355 | 26.783 | 22.635 | 1.00 | 20.92 |
| ATOM | 2087 | N | LEU | 1095 | 12.546 | 26.782 | 20.608 | 1.00 | 20.96 |
| ATOM | 2088 | H | LEU | 1095 | 13.286 | 26.321 | 20.157 | 1.00 | 25.00 |
| ATOM | 2089 | CA | LEU | 1095 | 11.877 | 27.954 | 19.932 | 1.00 | 19.31 |
| ATOM | 2090 | CB | LEU | 1095 | 12.352 | 28.101 | 18.469 | 1.00 | 19.46 |
| ATOM | 2091 | CG | LEU | 1095 | 13.869 | 28.484 | 18.319 | 1.00 | 23.37 |
| ATOM | 2092 | CD1 | LEU | 1095 | 14.318 | 28.888 | 16.867 | 1.00 | 22.59 |
| ATOM | 2093 | CD2 | LEU | 1095 | 14.110 | 29.641 | 19.243 | 1.00 | 22.26 |
| ATOM | 2094 | C | LEU | 1095 | 10.439 | 27.615 | 20.046 | 1.00 | 19.27 |
| ATOM | 2095 | O | LEU | 1095 | 9.735 | 28.332 | 20.730 | 1.00 | 18.43 |
| ATOM | 2096 | N | TRP | 1096 | 9.954 | 26.434 | 19.669 | 1.00 | 24.15 |
| ATOM | 2097 | H | TRP | 1096 | 10.592 | 25.786 | 19.311 | 1.00 | 25.00 |
| ATOM | 2098 | CA | TRP | 1096 | 8.478 | 25.995 | 19.800 | 1.00 | 22.47 |
| ATOM | 2099 | CB | TRP | 1096 | 8.190 | 24.546 | 19.128 | 1.00 | 22.13 |
| ATOM | 2100 | CG | TRP | 1096 | 6.767 | 24.130 | 18.872 | 1.00 | 16.90 |
| ATOM | 2101 | CD2 | TRP | 1096 | 5.795 | 23.644 | 19.815 | 1.00 | 17.98 |
| ATOM | 2102 | CE2 | TRP | 1096 | 4.595 | 23.495 | 19.094 | 1.00 | 14.01 |
| ATOM | 2103 | CE3 | TRP | 1096 | 5.815 | 23.316 | 21.203 | 1.00 | 16.81 |
| ATOM | 2104 | CD1 | TRP | 1096 | 6.102 | 24.234 | 17.657 | 1.00 | 13.34 |
| ATOM | 2105 | NE1 | TRP | 1096 | 4.802 | 23.836 | 17.845 | 1.00 | 17.85 |
| ATOM | 2106 | HE1 | TRP | 1096 | 4.149 | 23.735 | 17.116 | 1.00 | 25.00 |
| ATOM | 2107 | CZ2 | TRP | 1096 | 3.502 | 23.071 | 19.741 | 1.00 | 14.46 |
| ATOM | 2108 | CZ3 | TRP | 1096 | 4.647 | 22.865 | 21.841 | 1.00 | 12.70 |
| ATOM | 2109 | CH2 | TRP | 1096 | 3.492 | 22.756 | 21.113 | 1.00 | 14.17 |
| ATOM | 2110 | C | TRP | 1096 | 8.007 | 25.948 | 21.218 | 1.00 | 24.62 |
| ATOM | 2111 | O | TRP | 1096 | 6.845 | 26.294 | 21.409 | 1.00 | 27.52 |
| ATOM | 2112 | N | GLU | 1097 | 8.697 | 25.425 | 22.234 | 1.00 | 25.75 |
| ATOM | 2113 | H | GLU | 1097 | 9.498 | 24.921 | 22.052 | 1.00 | 25.00 |
| ATOM | 2114 | CA | GLU | 1097 | 8.098 | 25.540 | 23.632 | 1.00 | 25.34 |
| ATOM | 2115 | CB | GLU | 1097 | 8.847 | 24.897 | 24.751 | 1.00 | 22.63 |
| ATOM | 2116 | CG | GLU | 1097 | 9.257 | 23.549 | 24.137 | 1.00 | 20.77 |
| ATOM | 2117 | CD | GLU | 1097 | 10.209 | 22.892 | 25.143 | 1.00 | 15.97 |
| ATOM | 2118 | OE1 | GLU | 1097 | 9.737 | 22.634 | 26.180 | 1.00 | 13.60 |
| ATOM | 2119 | OE2 | GLU | 1097 | 11.439 | 22.780 | 24.852 | 1.00 | 14.82 |
| ATOM | 2120 | C | GLU | 1097 | 8.156 | 27.022 | 24.070 | 1.00 | 25.88 |
| ATOM | 2121 | O | GLU | 1097 | 7.359 | 27.357 | 24.958 | 1.00 | 26.93 |
| ATOM | 2122 | N | ILE | 1098 | 9.108 | 27.885 | 23.529 | 1.00 | 21.48 |
| ATOM | 2123 | H | ILE | 1098 | 9.801 | 27.479 | 22.996 | 1.00 | 25.00 |
| ATOM | 2124 | CA | ILE | 1098 | 9.135 | 29.264 | 23.877 | 1.00 | 18.13 |
| ATOM | 2125 | CB | ILE | 1098 | 10.458 | 29.856 | 23.351 | 1.00 | 17.86 |
| ATOM | 2126 | CG2 | ILE | 1098 | 10.348 | 31.364 | 23.570 | 1.00 | 17.72 |
| ATOM | 2127 | CG1 | ILE | 1098 | 11.726 | 29.488 | 24.257 | 1.00 | 16.28 |
| ATOM | 2128 | CD1 | ILE | 1098 | 12.903 | 30.374 | 23.817 | 1.00 | 12.05 |
| ATOM | 2129 | C | ILE | 1098 | 7.896 | 29.882 | 23.284 | 1.00 | 20.51 |
| ATOM | 2130 | O | ILE | 1098 | 6.866 | 30.200 | 23.937 | 1.00 | 20.69 |
| ATOM | 2131 | N | PHE | 1099 | 7.771 | 29.892 | 21.971 | 1.00 | 19.96 |
| ATOM | 2132 | H | PHE | 1099 | 8.450 | 29.434 | 21.448 | 1.00 | 25.00 |
| ATOM | 2133 | CA | PHE | 1099 | 6.533 | 30.505 | 21.349 | 1.00 | 21.69 |
| ATOM | 2134 | CB | PHE | 1099 | 6.911 | 30.788 | 19.896 | 1.00 | 23.88 |
| ATOM | 2135 | CG | PHE | 1099 | 8.028 | 31.751 | 19.887 | 1.00 | 25.83 |
| ATOM | 2136 | CD1 | PHE | 1099 | 9.322 | 31.328 | 19.659 | 1.00 | 25.49 |
| ATOM | 2137 | CD2 | PHE | 1099 | 7.749 | 33.130 | 20.049 | 1.00 | 29.46 |
| ATOM | 2138 | CE1 | PHE | 1099 | 10.368 | 32.272 | 19.589 | 1.00 | 29.46 |
| ATOM | 2139 | CE2 | PHE | 1099 | 8.776 | 34.090 | 19.973 | 1.00 | 28.26 |
| ATOM | 2140 | CZ | PHE | 1099 | 10.079 | 33.637 | 19.738 | 1.00 | 30.92 |
| ATOM | 2141 | C | PHE | 1099 | 5.104 | 29.835 | 21.438 | 1.00 | 23.83 |
| ATOM | 2142 | O | PHE | 1099 | 4.099 | 30.203 | 20.791 | 1.00 | 25.21 |
| ATOM | 2143 | N | SER | 1100 | 4.932 | 28.836 | 22.278 | 1.00 | 21.97 |
| ATOM | 2144 | H | SER | 1100 | 5.736 | 28.546 | 22.733 | 1.00 | 25.00 |
| ATOM | 2145 | CA | SER | 1100 | 3.669 | 28.146 | 22.611 | 1.00 | 21.78 |
| ATOM | 2146 | CB | SER | 1100 | 3.799 | 26.617 | 22.651 | 1.00 | 22.03 |
| ATOM | 2147 | OG | SER | 1100 | 4.910 | 25.998 | 23.400 | 1.00 | 28.71 |
| ATOM | 2148 | HG | SER | 1100 | 4.951 | 25.027 | 23.339 | 1.00 | 25.00 |
| ATOM | 2149 | C | SER | 1100 | 3.389 | 28.573 | 24.024 | 1.00 | 21.63 |
| ATOM | 2150 | O | SER | 1100 | 2.392 | 28.305 | 24.662 | 1.00 | 25.56 |
| ATOM | 2151 | N | LEU | 1101 | 4.381 | 29.158 | 24.672 | 1.00 | 20.91 |
| ATOM | 2152 | H | LEU | 1101 | 5.213 | 29.266 | 24.198 | 1.00 | 25.00 |
| ATOM | 2153 | CA | LEU | 1101 | 4.378 | 29.568 | 26.064 | 1.00 | 17.74 |
| ATOM | 2154 | CB | LEU | 1101 | 3.226 | 30.645 | 26.354 | 1.00 | 17.05 |
| ATOM | 2155 | CG | LEU | 1101 | 3.407 | 32.207 | 26.077 | 1.00 | 13.62 |
| ATOM | 2156 | CD1 | LEU | 1101 | 2.196 | 32.961 | 26.589 | 1.00 | 11.85 |
| ATOM | 2157 | CD2 | LEU | 1101 | 4.467 | 32.819 | 26.970 | 1.00 | 14.55 |
| ATOM | 2158 | C | LEU | 1101 | 4.296 | 28.324 | 26.983 | 1.00 | 16.90 |
| ATOM | 2159 | O | LEU | 1101 | 3.418 | 27.999 | 27.746 | 1.00 | 14.35 |
| ATOM | 2160 | N | GLY | 1102 | 5.303 | 27.514 | 26.807 | 1.00 | 17.34 |
| ATOM | 2161 | H | GLY | 1102 | 5.988 | 27.791 | 26.205 | 1.00 | 25.00 |
| ATOM | 2162 | CA | GLY | 1102 | 5.439 | 26.330 | 27.603 | 1.00 | 21.58 |
| ATOM | 2163 | C | GLY | 1102 | 4.478 | 25.222 | 27.346 | 1.00 | 23.22 |
| ATOM | 2164 | O | GLY | 1102 | 3.844 | 24.709 | 28.237 | 1.00 | 20.48 |
| ATOM | 2165 | N | ALA | 1103 | 4.329 | 24.779 | 26.127 | 1.00 | 26.61 |
| ATOM | 2166 | H | ALA | 1103 | 4.799 | 25.241 | 25.421 | 1.00 | 25.00 |
| ATOM | 2167 | CA | ALA | 1103 | 3.428 | 23.655 | 25.891 | 1.00 | 28.03 |
| ATOM | 2168 | CB | ALA | 1103 | 2.396 | 23.921 | 24.885 | 1.00 | 30.34 |
| ATOM | 2169 | C | ALA | 1103 | 4.163 | 22.505 | 25.382 | 1.00 | 27.29 |
| ATOM | 2170 | O | ALA | 1103 | 5.153 | 22.645 | 24.715 | 1.00 | 26.48 |
| ATOM | 2171 | N | SER | 1104 | 3.630 | 21.316 | 25.581 | 1.00 | 30.20 |
| ATOM | 2172 | H | SER | 1104 | 2.767 | 21.221 | 26.022 | 1.00 | 25.00 |
| ATOM | 2173 | CA | SER | 1104 | 4.431 | 20.159 | 25.160 | 1.00 | 28.46 |
| ATOM | 2174 | CB | SER | 1104 | 3.849 | 18.907 | 25.769 | 1.00 | 28.90 |
| ATOM | 2175 | OG | SER | 1104 | 4.860 | 18.448 | 26.700 | 1.00 | 35.21 |
| ATOM | 2176 | HG | SER | 1104 | 4.455 | 17.668 | 27.103 | 1.00 | 25.00 |
| ATOM | 2177 | C | SER | 1104 | 4.539 | 19.993 | 23.731 | 1.00 | 26.81 |
| ATOM | 2178 | O | SER | 1104 | 3.537 | 20.211 | 23.031 | 1.00 | 25.42 |
| ATOM | 2179 | N | PRO | 1105 | 5.723 | 19.663 | 23.221 | 1.00 | 26.41 |
| ATOM | 2180 | CD | PRO | 1105 | 7.019 | 19.946 | 23.805 | 1.00 | 26.89 |
| ATOM | 2181 | CA | PRO | 1105 | 5.807 | 19.153 | 21.796 | 1.00 | 28.86 |
| ATOM | 2182 | CB | PRO | 1105 | 7.316 | 18.708 | 21.670 | 1.00 | 28.48 |
| ATOM | 2183 | CG | PRO | 1105 | 8.021 | 19.744 | 22.599 | 1.00 | 25.77 |
| ATOM | 2184 | C | PRO | 1105 | 4.765 | 18.078 | 21.309 | 1.00 | 29.00 |
| ATOM | 2185 | O | PRO | 1105 | 3.939 | 17.526 | 22.045 | 1.00 | 27.42 |
| ATOM | 2186 | N | TYR | 1106 | 4.742 | 17.910 | 19.976 | 1.00 | 32.39 |
| ATOM | 2187 | H | TYR | 1106 | 5.403 | 18.431 | 19.461 | 1.00 | 25.00 |
| ATOM | 2188 | CA | TYR | 1106 | 3.884 | 16.976 | 19.212 | 1.00 | 32.09 |
| ATOM | 2189 | CB | TYR | 1106 | 4.746 | 15.733 | 19.042 | 1.00 | 28.37 |
| ATOM | 2190 | CG | TYR | 1106 | 5.967 | 16.150 | 18.342 | 1.00 | 22.33 |
| ATOM | 2191 | CD1 | TYR | 1106 | 7.252 | 16.190 | 18.885 | 1.00 | 18.74 |
| ATOM | 2192 | CE1 | TYR | 1106 | 8.345 | 16.610 | 18.056 | 1.00 | 19.73 |
| ATOM | 2193 | CD2 | TYR | 1106 | 5.735 | 16.506 | 17.023 | 1.00 | 20.46 |
| ATOM | 2194 | CE2 | TYR | 1106 | 6.822 | 16.890 | 16.240 | 1.00 | 19.47 |
| ATOM | 2195 | CZ | TYR | 1106 | 8.126 | 16.957 | 16.705 | 1.00 | 17.32 |
| ATOM | 2196 | OH | TYR | 1106 | 9.103 | 17.438 | 15.816 | 1.00 | 20.96 |
| ATOM | 2197 | HH | TYR | 1106 | 8.688 | 17.666 | 14.988 | 1.00 | 25.00 |
| ATOM | 2198 | C | TYR | 1106 | 2.605 | 16.792 | 19.916 | 1.00 | 37.70 |
| ATOM | 2199 | O | TYR | 1106 | 2.273 | 15.790 | 20.517 | 1.00 | 38.11 |
| ATOM | 2200 | N | PRO | 1107 | 1.783 | 17.783 | 19.848 | 1.00 | 42.80 |
| ATOM | 2201 | CD | PRO | 1107 | 1.611 | 18.612 | 18.644 | 1.00 | 44.75 |
| ATOM | 2202 | CA | PRO | 1107 | 0.598 | 17.844 | 20.767 | 1.00 | 46.86 |
| ATOM | 2203 | CB | PRO | 1107 | 0.116 | 19.270 | 20.559 | 1.00 | 45.44 |
| ATOM | 2204 | CG | PRO | 1107 | 0.906 | 19.812 | 19.332 | 1.00 | 44.85 |
| ATOM | 2205 | C | PRO | 1107 | −0.369 | 16.708 | 20.274 | 1.00 | 50.98 |
| ATOM | 2206 | O | PRO | 1107 | −0.617 | 16.646 | 19.049 | 1.00 | 52.62 |
| ATOM | 2207 | N | GLY | 1108 | −1.047 | 15.854 | 21.115 | 1.00 | 54.30 |
| ATOM | 2208 | H | GLY | 1108 | −1.024 | 16.010 | 22.086 | 1.00 | 25.00 |
| ATOM | 2209 | CA | GLY | 1108 | −1.848 | 14.673 | 20.564 | 1.00 | 56.17 |
| ATOM | 2210 | C | GLY | 1108 | −0.930 | 13.406 | 20.065 | 1.00 | 55.16 |
| ATOM | 2211 | O | GLY | 1108 | −1.254 | 12.251 | 20.353 | 1.00 | 57.87 |
| ATOM | 2212 | N | VAL | 1109 | 0.213 | 13.569 | 19.372 | 1.00 | 51.01 |
| ATOM | 2213 | H | VAL | 1109 | 0.480 | 14.505 | 19.217 | 1.00 | 25.00 |
| ATOM | 2214 | CA | VAL | 1109 | 1.089 | 12.470 | 18.906 | 1.00 | 46.70 |
| ATOM | 2215 | CB | VAL | 1109 | 2.181 | 13.177 | 18.055 | 1.00 | 44.68 |
| ATOM | 2216 | CG1 | VAL | 1109 | 3.200 | 12.293 | 17.360 | 1.00 | 40.01 |
| ATOM | 2217 | CG2 | VAL | 1109 | 1.350 | 13.971 | 17.051 | 1.00 | 44.02 |
| ATOM | 2218 | C | VAL | 1109 | 1.685 | 11.560 | 20.029 | 1.00 | 46.69 |
| ATOM | 2219 | O | VAL | 1109 | 2.390 | 11.991 | 20.940 | 1.00 | 46.19 |
| ATOM | 2220 | N | LYS | 1110 | 1.288 | 10.259 | 20.028 | 1.00 | 47.10 |
| ATOM | 2221 | H | LYS | 1110 | 0.568 | 10.029 | 19.394 | 1.00 | 25.00 |
| ATOM | 2222 | CA | LYS | 1110 | 1.812 | 9.213 | 20.979 | 1.00 | 47.38 |
| ATOM | 2223 | CB | LYS | 1110 | 1.111 | 7.822 | 20.919 | 1.00 | 47.34 |
| ATOM | 2224 | C | LYS | 1110 | 3.251 | 8.936 | 20.539 | 1.00 | 45.75 |
| ATOM | 2225 | O | LYS | 1110 | 3.490 | 8.574 | 19.392 | 1.00 | 44.86 |
| ATOM | 2226 | N | ILE | 1111 | 4.228 | 9.019 | 21.432 | 1.00 | 44.76 |
| ATOM | 2227 | H | ILE | 1111 | 4.028 | 9.340 | 22.326 | 1.00 | 25.00 |
| ATOM | 2228 | CA | ILE | 1111 | 5.590 | 8.881 | 20.958 | 1.00 | 45.21 |
| ATOM | 2229 | CB | ILE | 1111 | 6.517 | 9.854 | 21.851 | 1.00 | 40.71 |
| ATOM | 2230 | CG2 | ILE | 1111 | 7.931 | 9.971 | 21.353 | 1.00 | 36.51 |
| ATOM | 2231 | CG1 | ILE | 1111 | 5.951 | 11.270 | 21.767 | 1.00 | 39.93 |
| ATOM | 2232 | CD1 | ILE | 1111 | 6.119 | 11.730 | 20.297 | 1.00 | 37.57 |
| ATOM | 2233 | C | ILE | 1111 | 5.971 | 7.445 | 20.951 | 1.00 | 47.50 |
| ATOM | 2234 | O | ILE | 1111 | 6.297 | 6.783 | 21.942 | 1.00 | 48.98 |
| ATOM | 2235 | N | ASP | 1112 | 5.991 | 7.021 | 19.698 | 1.00 | 48.84 |
| ATOM | 2236 | H | ASP | 1112 | 5.875 | 7.709 | 19.016 | 1.00 | 25.00 |
| ATOM | 2237 | CA | ASP | 1112 | 6.316 | 5.643 | 19.341 | 1.00 | 48.91 |
| ATOM | 2238 | CB | ASP | 1112 | 5.025 | 4.930 | 19.312 | 1.00 | 45.81 |
| ATOM | 2239 | CG | ASP | 1112 | 4.151 | 5.510 | 18.248 | 1.00 | 43.96 |
| ATOM | 2240 | OD1 | ASP | 1112 | 2.993 | 5.638 | 18.545 | 1.00 | 43.60 |
| ATOM | 2241 | OD2 | ASP | 1112 | 4.564 | 5.772 | 17.128 | 1.00 | 42.43 |
| ATOM | 2242 | C | ASP | 1112 | 7.023 | 5.463 | 18.024 | 1.00 | 49.98 |
| ATOM | 2243 | O | ASP | 1112 | 7.145 | 6.440 | 17.292 | 1.00 | 52.43 |
| ATOM | 2244 | N | GLU | 1113 | 7.203 | 4.195 | 17.591 | 1.00 | 50.32 |
| ATOM | 2245 | H | GLU | 1113 | 6.832 | 3.534 | 18.197 | 1.00 | 25.00 |
| ATOM | 2246 | CA | GLU | 1113 | 7.854 | 3.742 | 16.301 | 1.00 | 50.11 |
| ATOM | 2247 | CB | GLU | 1113 | 7.488 | 2.281 | 16.041 | 1.00 | 51.85 |
| ATOM | 2248 | C | GLU | 1113 | 7.409 | 4.558 | 15.079 | 1.00 | 48.46 |
| ATOM | 2249 | O | GLU | 1113 | 8.165 | 4.958 | 14.211 | 1.00 | 45.03 |
| ATOM | 2250 | N | GLU | 1114 | 6.137 | 4.872 | 15.040 | 1.00 | 49.72 |
| ATOM | 2251 | H | GLU | 1114 | 5.538 | 4.518 | 15.719 | 1.00 | 25.00 |
| ATOM | 2252 | CA | GLU | 1114 | 5.564 | 5.656 | 13.945 | 1.00 | 53.60 |
| ATOM | 2253 | CB | GLU | 1114 | 4.070 | 5.333 | 13.944 | 1.00 | 58.05 |
| ATOM | 2254 | CG | GLU | 1114 | 3.999 | 3.759 | 13.746 | 1.00 | 62.96 |
| ATOM | 2255 | CD | GLU | 1114 | 4.532 | 3.320 | 12.385 | 1.00 | 64.21 |
| ATOM | 2256 | OE1 | GLU | 1114 | 5.726 | 3.081 | 12.206 | 1.00 | 63.41 |
| ATOM | 2257 | OE2 | GLU | 1114 | 3.721 | 3.215 | 11.473 | 1.00 | 65.13 |
| ATOM | 2258 | C | GLU | 1114 | 5.879 | 7.127 | 14.037 | 1.00 | 54.15 |
| ATOM | 2259 | O | GLU | 1114 | 6.219 | 7.747 | 13.026 | 1.00 | 54.04 |
| ATOM | 2260 | N | PHE | 1115 | 5.811 | 7.684 | 15.287 | 1.00 | 53.49 |
| ATOM | 2261 | H | PHE | 1115 | 5.510 | 7.147 | 16.040 | 1.00 | 25.00 |
| ATOM | 2262 | CA | PHE | 1115 | 6.170 | 9.084 | 15.568 | 1.00 | 49.34 |
| ATOM | 2263 | CB | PHE | 1115 | 6.093 | 9.294 | 17.128 | 1.00 | 48.87 |
| ATOM | 2264 | CG | PHE | 1115 | 6.926 | 10.453 | 17.470 | 1.00 | 50.12 |
| ATOM | 2265 | CD1 | PHE | 1115 | 8.270 | 10.258 | 17.874 | 1.00 | 48.17 |
| ATOM | 2266 | CD2 | PHE | 1115 | 6.419 | 11.752 | 17.184 | 1.00 | 50.39 |
| ATOM | 2267 | CE1 | PHE | 1115 | 9.120 | 11.366 | 17.968 | 1.00 | 46.40 |
| ATOM | 2268 | CE2 | PHE | 1115 | 7.281 | 12.846 | 17.289 | 1.00 | 47.92 |
| ATOM | 2269 | CZ | PHE | 1115 | 8.618 | 12.637 | 17.675 | 1.00 | 45.53 |
| ATOM | 2270 | C | PHE | 1115 | 7.559 | 9.228 | 14.968 | 1.00 | 48.44 |
| ATOM | 2271 | O | PHE | 1115 | 7.691 | 10.060 | 14.073 | 1.00 | 47.06 |
| ATOM | 2272 | N | CYS | 1116 | 8.533 | 8.343 | 15.324 | 1.00 | 46.82 |
| ATOM | 2273 | H | CYS | 1116 | 8.265 | 7.578 | 15.881 | 1.00 | 25.00 |
| ATOM | 2274 | CA | CYS | 1116 | 9.910 | 8.400 | 14.756 | 1.00 | 48.49 |
| ATOM | 2275 | CB | CYS | 1116 | 10.789 | 7.303 | 15.313 | 1.00 | 48.42 |
| ATOM | 2276 | SG | CYS | 1116 | 10.678 | 7.110 | 17.073 | 1.00 | 54.37 |
| ATOM | 2277 | C | CYS | 1116 | 10.056 | 8.264 | 13.214 | 1.00 | 49.91 |
| ATOM | 2278 | O | CYS | 1116 | 10.852 | 8.896 | 12.499 | 1.00 | 48.30 |
| ATOM | 2279 | N | ARG | 1117 | 9.346 | 7.318 | 12.636 | 1.00 | 52.31 |
| ATOM | 2280 | H | ARG | 1117 | 8.754 | 6.782 | 13.178 | 1.00 | 25.00 |
| ATOM | 2281 | CA | ARG | 1117 | 9.440 | 7.132 | 11.176 | 1.00 | 53.98 |
| ATOM | 2282 | CB | ARG | 1117 | 8.965 | 5.653 | 10.963 | 1.00 | 55.45 |
| ATOM | 2283 | CG | ARG | 1117 | 8.515 | 5.202 | 9.520 | 1.00 | 55.43 |
| ATOM | 2284 | CD | ARG | 1117 | 6.981 | 4.827 | 9.517 | 1.00 | 52.70 |
| ATOM | 2285 | NE | ARG | 1117 | 6.223 | 5.754 | 8.693 | 1.00 | 50.68 |
| ATOM | 2286 | HE | ARG | 1117 | 6.694 | 6.265 | 8.013 | 1.00 | 25.00 |
| ATOM | 2287 | CZ | ARG | 1117 | 4.926 | 5.932 | 8.910 | 1.00 | 53.22 |
| ATOM | 2288 | NH1 | ARG | 1117 | 4.208 | 6.767 | 8.163 | 1.00 | 55.02 |
| ATOM | 2289 | HH11 | ARG | 1117 | 4.654 | 7.285 | 7.429 | 1.00 | 25.00 |
| ATOM | 2290 | HH12 | ARG | 1117 | 3.232 | 6.893 | 8.351 | 1.00 | 25.00 |
| ATOM | 2291 | NH2 | ARG | 1117 | 4.289 | 5.274 | 9.873 | 1.00 | 55.66 |
| ATOM | 2292 | HH21 | ARG | 1117 | 4.770 | 4.610 | 10.448 | 1.00 | 25.00 |
| ATOM | 2293 | HH22 | ARG | 1117 | 3.308 | 5.425 | 10.007 | 1.00 | 25.00 |
| ATOM | 2294 | C | ARG | 1117 | 8.664 | 8.249 | 10.416 | 1.00 | 53.83 |
| ATOM | 2295 | O | ARG | 1117 | 9.137 | 8.765 | 9.394 | 1.00 | 53.03 |
| ATOM | 2296 | N | ARG | 1118 | 7.459 | 8.631 | 10.852 | 1.00 | 52.45 |
| ATOM | 2297 | H | ARG | 1118 | 6.996 | 8.150 | 11.543 | 1.00 | 25.00 |
| ATOM | 2298 | CA | ARG | 1118 | 6.767 | 9.699 | 10.163 | 1.00 | 54.40 |
| ATOM | 2299 | CB | ARG | 1118 | 5.423 | 9.740 | 10.893 | 1.00 | 53.94 |
| ATOM | 2300 | CG | ARG | 1118 | 4.019 | 9.785 | 10.222 | 1.00 | 55.32 |
| ATOM | 2301 | CD | ARG | 1118 | 2.844 | 9.729 | 11.280 | 1.00 | 58.84 |
| ATOM | 2302 | NE | ARG | 1118 | 3.322 | 10.646 | 12.342 | 1.00 | 62.73 |
| ATOM | 2303 | HE | ARG | 1118 | 3.366 | 11.594 | 12.107 | 1.00 | 25.00 |
| ATOM | 2304 | CZ | ARG | 1118 | 3.682 | 10.363 | 13.618 | 1.00 | 64.33 |
| ATOM | 2305 | NH1 | ARG | 1118 | 4.168 | 11.379 | 14.404 | 1.00 | 64.89 |
| ATOM | 2306 | HH11 | ARG | 1118 | 4.238 | 12.308 | 14.046 | 1.00 | 25.00 |
| ATOM | 2307 | HH12 | ARG | 1118 | 4.447 | 11.172 | 15.343 | 1.00 | 25.00 |
| ATOM | 2308 | NH2 | ARG | 1118 | 3.494 | 9.128 | 14.134 | 1.00 | 64.62 |
| ATOM | 2309 | HH21 | ARG | 1118 | 3.092 | 8.393 | 13.586 | 1.00 | 25.00 |
| ATOM | 2310 | HH22 | ARG | 1118 | 3.780 | 8.928 | 15.070 | 1.00 | 25.00 |
| ATOM | 2311 | C | ARG | 1118 | 7.786 | 10.879 | 10.336 | 1.00 | 56.01 |
| ATOM | 2312 | O | ARG | 1118 | 8.217 | 11.495 | 9.366 | 1.00 | 57.22 |
| ATOM | 2313 | N | LEU | 1119 | 8.387 | 11.123 | 11.519 | 1.00 | 57.58 |
| ATOM | 2314 | H | LEU | 1119 | 8.114 | 10.639 | 12.319 | 1.00 | 25.00 |
| ATOM | 2315 | CA | LEU | 1119 | 9.390 | 12.229 | 11.599 | 1.00 | 58.82 |
| ATOM | 2316 | CB | LEU | 1119 | 10.047 | 12.484 | 13.045 | 1.00 | 59.62 |
| ATOM | 2317 | CG | LEU | 1119 | 9.475 | 13.548 | 14.069 | 1.00 | 58.45 |
| ATOM | 2318 | CD1 | LEU | 1119 | 10.565 | 14.576 | 14.242 | 1.00 | 59.12 |
| ATOM | 2319 | CD2 | LEU | 1119 | 8.163 | 14.230 | 13.637 | 1.00 | 59.25 |
| ATOM | 2320 | C | LEU | 1119 | 10.566 | 11.971 | 10.659 | 1.00 | 57.85 |
| ATOM | 2321 | O | LEU | 1119 | 10.864 | 12.839 | 9.855 | 1.00 | 58.04 |
| ATOM | 2322 | N | LYS | 1120 | 11.257 | 10.840 | 10.708 | 1.00 | 57.73 |
| ATOM | 2323 | H | LYS | 1120 | 10.948 | 10.171 | 11.329 | 1.00 | 25.00 |
| ATOM | 2324 | CA | LYS | 1120 | 12.391 | 10.588 | 9.799 | 1.00 | 57.69 |
| ATOM | 2325 | CB | LYS | 1120 | 12.834 | 9.108 | 9.957 | 1.00 | 57.51 |
| ATOM | 2326 | CG | LYS | 1120 | 14.353 | 9.105 | 10.188 | 1.00 | 57.64 |
| ATOM | 2327 | CD | LYS | 1120 | 15.329 | 7.824 | 10.084 | 1.00 | 57.73 |
| ATOM | 2328 | CE | LYS | 1120 | 16.887 | 8.231 | 10.297 | 1.00 | 52.79 |
| ATOM | 2329 | NZ | LYS | 1120 | 17.795 | 7.131 | 10.048 | 1.00 | 49.63 |
| ATOM | 2330 | HZ1 | LYS | 1120 | 17.491 | 6.657 | 9.176 | 1.00 | 25.00 |
| ATOM | 2331 | HZ2 | LYS | 1120 | 17.718 | 6.453 | 10.831 | 1.00 | 25.00 |
| ATOM | 2332 | HZ3 | LYS | 1120 | 18.749 | 7.517 | 9.975 | 1.00 | 25.00 |
| ATOM | 2333 | C | LYS | 1120 | 12.030 | 10.888 | 8.289 | 1.00 | 58.03 |
| ATOM | 2334 | O | LYS | 1120 | 12.889 | 11.283 | 7.464 | 1.00 | 59.30 |
| ATOM | 2335 | N | GLU | 1121 | 10.725 | 10.662 | 7.910 | 1.00 | 56.98 |
| ATOM | 2336 | H | GLU | 1121 | 10.095 | 10.342 | 8.600 | 1.00 | 25.00 |
| ATOM | 2337 | CA | GLU | 1121 | 10.234 | 10.888 | 6.541 | 1.00 | 53.98 |
| ATOM | 2338 | CB | GLU | 1121 | 9.251 | 9.700 | 6.315 | 1.00 | 53.54 |
| ATOM | 2339 | CG | GLU | 1121 | 7.767 | 10.037 | 6.359 | 1.00 | 56.71 |
| ATOM | 2340 | CD | GLU | 1121 | 6.735 | 8.874 | 6.380 | 1.00 | 58.55 |
| ATOM | 2341 | OE1 | GLU | 1121 | 5.524 | 9.153 | 6.229 | 1.00 | 58.21 |
| ATOM | 2342 | OE2 | GLU | 1121 | 7.112 | 7.706 | 6.589 | 1.00 | 59.37 |
| ATOM | 2343 | C | GLU | 1121 | 9.678 | 12.311 | 6.340 | 1.00 | 52.78 |
| ATOM | 2344 | O | GLU | 1121 | 8.972 | 12.639 | 5.390 | 1.00 | 53.41 |
| ATOM | 2345 | N | GLY | 1122 | 10.041 | 13.269 | 7.182 | 1.00 | 50.57 |
| ATOM | 2346 | H | GLY | 1122 | 10.582 | 13.037 | 7.961 | 1.00 | 25.00 |
| ATOM | 2347 | CA | GLY | 1122 | 9.574 | 14.643 | 7.016 | 1.00 | 47.68 |
| ATOM | 2348 | C | GLY | 1122 | 8.455 | 15.216 | 7.894 | 1.00 | 47.58 |
| ATOM | 2349 | O | GLY | 1122 | 8.120 | 16.361 | 7.663 | 1.00 | 47.91 |
| ATOM | 2350 | N | THR | 1123 | 7.679 | 14.579 | 8.755 | 1.00 | 46.51 |
| ATOM | 2351 | H | THR | 1123 | 7.716 | 13.608 | 8.795 | 1.00 | 25.00 |
| ATOM | 2352 | CA | THR | 1123 | 6.655 | 15.342 | 9.574 | 1.00 | 46.74 |
| ATOM | 2353 | CB | THR | 1123 | 5.801 | 14.371 | 10.368 | 1.00 | 48.37 |
| ATOM | 2354 | OG1 | THR | 1123 | 5.993 | 13.028 | 9.819 | 1.00 | 49.06 |
| ATOM | 2355 | HG1 | THR | 1123 | 5.496 | 12.413 | 10.364 | 1.00 | 25.00 |
| ATOM | 2356 | CG2 | THR | 1123 | 4.287 | 14.815 | 10.266 | 1.00 | 48.12 |
| ATOM | 2357 | C | THR | 1123 | 7.349 | 16.354 | 10.570 | 1.00 | 44.91 |
| ATOM | 2358 | O | THR | 1123 | 8.478 | 16.139 | 11.037 | 1.00 | 42.37 |
| ATOM | 2359 | N | ARG | 1124 | 6.716 | 17.479 | 10.940 | 1.00 | 41.75 |
| ATOM | 2360 | H | ARG | 1124 | 5.763 | 17.557 | 10.755 | 1.00 | 25.00 |
| ATOM | 2361 | CA | ARG | 1124 | 7.355 | 18.585 | 11.765 | 1.00 | 39.45 |
| ATOM | 2362 | CB | ARG | 1124 | 7.997 | 19.751 | 10.971 | 1.00 | 33.99 |
| ATOM | 2363 | CG | ARG | 1124 | 9.368 | 19.573 | 10.240 | 1.00 | 27.79 |
| ATOM | 2364 | CD | ARG | 1124 | 10.252 | 18.632 | 10.886 | 1.00 | 26.97 |
| ATOM | 2365 | NE | ARG | 1124 | 11.218 | 18.330 | 9.855 | 1.00 | 30.02 |
| ATOM | 2366 | HE | ARG | 1124 | 11.712 | 19.052 | 9.422 | 1.00 | 25.00 |
| ATOM | 2367 | CZ | ARG | 1124 | 11.532 | 17.039 | 9.626 | 1.00 | 33.89 |
| ATOM | 2368 | NH1 | ARG | 1124 | 10.969 | 16.046 | 10.333 | 1.00 | 34.33 |
| ATOM | 2369 | HH11 | ARG | 1124 | 10.302 | 16.277 | 11.031 | 1.00 | 25.00 |
| ATOM | 2370 | HH12 | ARG | 1124 | 11.226 | 15.105 | 10.151 | 1.00 | 25.00 |
| ATOM | 2371 | NH2 | ARG | 1124 | 12.439 | 16.717 | 8.681 | 1.00 | 33.78 |
| ATOM | 2372 | HH21 | ARG | 1124 | 12.890 | 17.424 | 8.135 | 1.00 | 25.00 |
| ATOM | 2373 | HH22 | ARG | 1124 | 12.695 | 15.764 | 8.523 | 1.00 | 25.00 |
| ATOM | 2374 | C | ARG | 1124 | 6.328 | 19.286 | 12.599 | 1.00 | 39.04 |
| ATOM | 2375 | O | ARG | 1124 | 5.181 | 19.286 | 12.161 | 1.00 | 41.44 |
| ATOM | 2376 | N | MET | 1125 | 6.640 | 19.952 | 13.717 | 1.00 | 36.82 |
| ATOM | 2377 | H | MET | 1125 | 7.564 | 20.216 | 13.943 | 1.00 | 25.00 |
| ATOM | 2378 | CA | MET | 1125 | 5.416 | 20.427 | 14.393 | 1.00 | 32.81 |
| ATOM | 2379 | CB | MET | 1125 | 5.996 | 20.742 | 15.748 | 1.00 | 32.01 |
| ATOM | 2380 | CG | MET | 1125 | 5.207 | 20.096 | 16.820 | 1.00 | 26.26 |
| ATOM | 2381 | SD | MET | 1125 | 5.867 | 20.612 | 18.376 | 1.00 | 25.75 |
| ATOM | 2382 | CE | MET | 1125 | 7.621 | 20.308 | 18.211 | 1.00 | 10.01 |
| ATOM | 2383 | C | MET | 1125 | 4.508 | 21.534 | 13.727 | 1.00 | 34.63 |
| ATOM | 2384 | O | MET | 1125 | 4.753 | 22.165 | 12.687 | 1.00 | 31.81 |
| ATOM | 2385 | N | ARG | 1126 | 3.344 | 21.719 | 14.348 | 1.00 | 35.77 |
| ATOM | 2386 | H | ARG | 1126 | 3.123 | 21.159 | 15.125 | 1.00 | 25.00 |
| ATOM | 2387 | CA | ARG | 1126 | 2.407 | 22.758 | 13.823 | 1.00 | 40.57 |
| ATOM | 2388 | CB | ARG | 1126 | 0.964 | 22.625 | 14.388 | 1.00 | 46.37 |
| ATOM | 2389 | CG | ARG | 1126 | 1.007 | 22.494 | 15.995 | 1.00 | 54.46 |
| ATOM | 2390 | CD | ARG | 1126 | 0.273 | 23.651 | 16.777 | 1.00 | 54.56 |
| ATOM | 2391 | NE | ARG | 1126 | 0.846 | 24.015 | 18.119 | 1.00 | 56.78 |
| ATOM | 2392 | HE | ARG | 1126 | 1.771 | 23.736 | 18.314 | 1.00 | 25.00 |
| ATOM | 2393 | CZ | ARG | 1126 | 0.186 | 24.741 | 19.039 | 1.00 | 56.64 |
| ATOM | 2394 | NH1 | ARG | 1126 | 0.726 | 25.073 | 20.244 | 1.00 | 54.98 |
| ATOM | 2395 | HH11 | ARG | 1126 | 1.653 | 24.774 | 20.464 | 1.00 | 25.00 |
| ATOM | 2396 | HH12 | ARG | 1126 | 0.175 | 25.566 | 20.915 | 1.00 | 25.00 |
| ATOM | 2397 | NH2 | ARG | 1126 | −1.059 | 25.060 | 18.683 | 1.00 | 57.97 |
| ATOM | 2398 | HH21 | ARG | 1126 | −1.392 | 24.772 | 17.789 | 1.00 | 25.00 |
| ATOM | 2399 | HH22 | ARG | 1126 | −1.659 | 25.560 | 19.300 | 1.00 | 25.00 |
| ATOM | 2400 | C | ARG | 1126 | 2.973 | 24.094 | 14.258 | 1.00 | 39.81 |
| ATOM | 2401 | O | ARG | 1126 | 4.009 | 24.098 | 14.942 | 1.00 | 39.78 |
| ATOM | 2402 | N | ALA | 1127 | 2.357 | 25.228 | 13.876 | 1.00 | 37.48 |
| ATOM | 2403 | H | ALA | 1127 | 1.494 | 25.203 | 13.407 | 1.00 | 25.00 |
| ATOM | 2404 | CA | ALA | 1127 | 2.980 | 26.502 | 14.295 | 1.00 | 34.48 |
| ATOM | 2405 | CB | ALA | 1127 | 2.882 | 27.601 | 13.198 | 1.00 | 35.65 |
| ATOM | 2406 | C | ALA | 1127 | 2.342 | 27.003 | 15.548 | 1.00 | 29.84 |
| ATOM | 2407 | O | ALA | 1127 | 1.108 | 26.876 | 15.694 | 1.00 | 28.74 |
| ATOM | 2408 | N | PRO | 1128 | 3.182 | 27.404 | 16.512 | 1.00 | 24.48 |
| ATOM | 2409 | CD | PRO | 1128 | 4.609 | 27.302 | 16.540 | 1.00 | 24.29 |
| ATOM | 2410 | CA | PRO | 1128 | 2.546 | 27.519 | 17.794 | 1.00 | 25.82 |
| ATOM | 2411 | CB | PRO | 1128 | 3.616 | 27.198 | 18.784 | 1.00 | 24.12 |
| ATOM | 2412 | CG | PRO | 1128 | 4.836 | 27.627 | 18.027 | 1.00 | 23.44 |
| ATOM | 2413 | C | PRO | 1128 | 1.802 | 28.839 | 18.026 | 1.00 | 27.63 |
| ATOM | 2414 | O | PRO | 1128 | 2.011 | 29.850 | 17.388 | 1.00 | 26.07 |
| ATOM | 2415 | N | ASP | 1129 | 0.841 | 28.826 | 18.915 | 1.00 | 29.07 |
| ATOM | 2416 | H | ASP | 1129 | 0.744 | 27.989 | 19.400 | 1.00 | 25.00 |
| ATOM | 2417 | CA | ASP | 1129 | 0.018 | 29.943 | 19.222 | 1.00 | 31.21 |
| ATOM | 2418 | CB | ASP | 1129 | −0.490 | 29.702 | 20.586 | 1.00 | 31.94 |
| ATOM | 2419 | CG | ASP | 1129 | −1.565 | 28.689 | 20.656 | 1.00 | 32.62 |
| ATOM | 2420 | OD1 | ASP | 1129 | −2.032 | 28.365 | 21.769 | 1.00 | 33.21 |
| ATOM | 2421 | OD2 | ASP | 1129 | −1.938 | 28.275 | 19.569 | 1.00 | 34.16 |
| ATOM | 2422 | C | ASP | 1129 | 0.498 | 31.391 | 19.161 | 1.00 | 33.97 |
| ATOM | 2423 | O | ASP | 1129 | −0.372 | 32.196 | 18.746 | 1.00 | 36.72 |
| ATOM | 2424 | N | TYR | 1130 | 1.772 | 31.717 | 19.700 | 1.00 | 31.76 |
| ATOM | 2425 | H | TYR | 1130 | 2.328 | 30.970 | 20.038 | 1.00 | 25.00 |
| ATOM | 2426 | CA | TYR | 1130 | 2.345 | 33.115 | 19.776 | 1.00 | 24.26 |
| ATOM | 2427 | CB | TYR | 1130 | 2.623 | 33.290 | 21.268 | 1.00 | 20.42 |
| ATOM | 2428 | CG | TYR | 1130 | 1.553 | 32.749 | 22.122 | 1.00 | 16.76 |
| ATOM | 2429 | CD1 | TYR | 1130 | 1.697 | 31.521 | 22.667 | 1.00 | 17.35 |
| ATOM | 2430 | CE1 | TYR | 1130 | 0.687 | 30.995 | 23.525 | 1.00 | 18.22 |
| ATOM | 2431 | CD2 | TYR | 1130 | 0.408 | 33.466 | 22.414 | 1.00 | 16.69 |
| ATOM | 2432 | CE2 | TYR | 1130 | −0.625 | 32.952 | 23.269 | 1.00 | 18.49 |
| ATOM | 2433 | CZ | TYR | 1130 | −0.469 | 31.674 | 23.841 | 1.00 | 19.13 |
| ATOM | 2434 | OH | TYR | 1130 | −1.405 | 31.084 | 24.714 | 1.00 | 21.04 |
| ATOM | 2435 | HH | TYR | 1130 | −2.124 | 31.690 | 24.895 | 1.00 | 25.00 |
| ATOM | 2436 | C | TYR | 1130 | 3.538 | 33.400 | 18.894 | 1.00 | 20.99 |
| ATOM | 2437 | O | TYR | 1130 | 4.234 | 34.390 | 19.060 | 1.00 | 22.77 |
| ATOM | 2438 | N | THR | 1131 | 3.896 | 32.519 | 18.001 | 1.00 | 18.89 |
| ATOM | 2439 | H | THR | 1131 | 3.419 | 31.668 | 17.971 | 1.00 | 25.00 |
| ATOM | 2440 | CA | THR | 1131 | 5.026 | 32.830 | 17.116 | 1.00 | 21.33 |
| ATOM | 2441 | CB | THR | 1131 | 5.363 | 31.520 | 16.306 | 1.00 | 21.78 |
| ATOM | 2442 | OG1 | THR | 1131 | 6.443 | 31.565 | 15.340 | 1.00 | 20.81 |
| ATOM | 2443 | HG1 | THR | 1131 | 6.457 | 30.727 | 14.873 | 1.00 | 25.00 |
| ATOM | 2444 | CG2 | THR | 1131 | 4.169 | 31.255 | 15.507 | 1.00 | 17.15 |
| ATOM | 2445 | C | THR | 1131 | 4.747 | 34.013 | 16.157 | 1.00 | 27.77 |
| ATOM | 2446 | O | THR | 1131 | 3.655 | 34.627 | 16.077 | 1.00 | 28.78 |
| ATOM | 2447 | N | THR | 1132 | 5.822 | 34.382 | 15.420 | 1.00 | 30.96 |
| ATOM | 2448 | H | THR | 1132 | 6.644 | 33.871 | 15.563 | 1.00 | 25.00 |
| ATOM | 2449 | CA | THR | 1132 | 5.913 | 35.431 | 14.368 | 1.00 | 32.58 |
| ATOM | 2450 | CB | THR | 1132 | 7.118 | 36.476 | 14.467 | 1.00 | 32.35 |
| ATOM | 2451 | OG1 | THR | 1132 | 8.327 | 35.737 | 14.466 | 1.00 | 33.27 |
| ATOM | 2452 | HG1 | THR | 1132 | 9.052 | 36.342 | 14.664 | 1.00 | 25.00 |
| ATOM | 2453 | CG2 | THR | 1132 | 7.052 | 37.390 | 15.647 | 1.00 | 30.38 |
| ATOM | 2454 | C | THR | 1132 | 6.240 | 34.614 | 13.084 | 1.00 | 36.70 |
| ATOM | 2455 | O | THR | 1132 | 6.854 | 33.526 | 13.096 | 1.00 | 36.52 |
| ATOM | 2456 | N | PRO | 1133 | 5.776 | 35.067 | 11.930 | 1.00 | 37.80 |
| ATOM | 2457 | CD | PRO | 1133 | 4.486 | 35.800 | 11.751 | 1.00 | 38.91 |
| ATOM | 2458 | CA | PRO | 1133 | 6.132 | 34.309 | 10.736 | 1.00 | 36.78 |
| ATOM | 2459 | CB | PRO | 1133 | 5.573 | 35.315 | 9.691 | 1.00 | 37.46 |
| ATOM | 2460 | CG | PRO | 1133 | 4.156 | 35.434 | 10.307 | 1.00 | 37.99 |
| ATOM | 2461 | C | PRO | 1133 | 7.565 | 33.800 | 10.631 | 1.00 | 35.99 |
| ATOM | 2462 | O | PRO | 1133 | 7.873 | 32.618 | 10.531 | 1.00 | 35.88 |
| ATOM | 2463 | N | GLU | 1134 | 8.519 | 34.673 | 10.701 | 1.00 | 37.31 |
| ATOM | 2464 | H | GLU | 1134 | 8.268 | 35.604 | 10.823 | 1.00 | 25.00 |
| ATOM | 2465 | CA | GLU | 1134 | 9.910 | 34.260 | 10.608 | 1.00 | 36.30 |
| ATOM | 2466 | CB | GLU | 1134 | 10.753 | 35.601 | 10.381 | 1.00 | 38.28 |
| ATOM | 2467 | CG | GLU | 1134 | 10.423 | 36.936 | 11.138 | 1.00 | 42.08 |
| ATOM | 2468 | CD | GLU | 1134 | 9.129 | 37.649 | 10.650 | 1.00 | 44.27 |
| ATOM | 2469 | OE1 | GLU | 1134 | 8.128 | 37.802 | 11.384 | 1.00 | 45.26 |
| ATOM | 2470 | OE2 | GLU | 1134 | 9.105 | 38.040 | 9.481 | 1.00 | 51.15 |
| ATOM | 2471 | C | GLU | 1134 | 10.270 | 33.412 | 11.838 | 1.00 | 36.73 |
| ATOM | 2472 | O | GLU | 1134 | 11.175 | 32.622 | 11.644 | 1.00 | 35.14 |
| ATOM | 2473 | N | MET | 1135 | 9.663 | 33.522 | 13.085 | 1.00 | 37.14 |
| ATOM | 2474 | H | MET | 1135 | 8.915 | 34.157 | 13.153 | 1.00 | 25.00 |
| ATOM | 2475 | CA | MET | 1135 | 10.041 | 32.634 | 14.225 | 1.00 | 36.24 |
| ATOM | 2476 | CB | MET | 1135 | 9.456 | 33.084 | 15.572 | 1.00 | 35.92 |
| ATOM | 2477 | CG | MET | 1135 | 10.532 | 33.895 | 16.436 | 1.00 | 39.05 |
| ATOM | 2478 | SD | MET | 1135 | 12.400 | 33.487 | 16.546 | 1.00 | 42.26 |
| ATOM | 2479 | CE | MET | 1135 | 12.171 | 31.734 | 16.711 | 1.00 | 41.30 |
| ATOM | 2480 | C | MET | 1135 | 9.605 | 31.196 | 13.896 | 1.00 | 38.58 |
| ATOM | 2481 | O | MET | 1135 | 10.459 | 30.296 | 13.999 | 1.00 | 40.87 |
| ATOM | 2482 | N | TYR | 1136 | 8.365 | 30.837 | 13.460 | 1.00 | 38.46 |
| ATOM | 2483 | H | TYR | 1136 | 7.663 | 31.532 | 13.428 | 1.00 | 25.00 |
| ATOM | 2484 | CA | TYR | 1136 | 8.123 | 29.399 | 12.980 | 1.00 | 36.26 |
| ATOM | 2485 | CB | TYR | 1136 | 6.693 | 29.301 | 12.441 | 1.00 | 41.32 |
| ATOM | 2486 | CG | TYR | 1136 | 6.375 | 27.929 | 12.062 | 1.00 | 45.56 |
| ATOM | 2487 | CD1 | TYR | 1136 | 6.319 | 26.943 | 13.063 | 1.00 | 46.60 |
| ATOM | 2488 | CE1 | TYR | 1136 | 6.095 | 25.598 | 12.686 | 1.00 | 49.46 |
| ATOM | 2489 | CD2 | TYR | 1136 | 6.209 | 27.625 | 10.691 | 1.00 | 47.00 |
| ATOM | 2490 | CE2 | TYR | 1136 | 5.989 | 26.279 | 10.322 | 1.00 | 48.28 |
| ATOM | 2491 | CZ | TYR | 1136 | 5.924 | 25.245 | 11.314 | 1.00 | 47.90 |
| ATOM | 2492 | OH | TYR | 1136 | 5.549 | 23.952 | 10.960 | 1.00 | 44.91 |
| ATOM | 2493 | HH | TYR | 1136 | 5.342 | 23.945 | 10.026 | 1.00 | 25.00 |
| ATOM | 2494 | C | TYR | 1136 | 9.094 | 28.899 | 11.845 | 1.00 | 35.03 |
| ATOM | 2495 | O | TYR | 1136 | 9.546 | 27.754 | 11.776 | 1.00 | 33.28 |
| ATOM | 2496 | N | GLN | 1137 | 9.426 | 29.633 | 10.815 | 1.00 | 34.97 |
| ATOM | 2497 | H | GLN | 1137 | 9.132 | 30.561 | 10.766 | 1.00 | 25.00 |
| ATOM | 2498 | CA | GLN | 1137 | 10.359 | 29.082 | 9.797 | 1.00 | 34.42 |
| ATOM | 2499 | CB | GLN | 1137 | 10.324 | 29.940 | 8.516 | 1.00 | 39.53 |
| ATOM | 2500 | CG | GLN | 1137 | 11.277 | 29.408 | 7.328 | 1.00 | 40.32 |
| ATOM | 2501 | CD | GLN | 1137 | 10.705 | 28.264 | 6.490 | 1.00 | 39.24 |
| ATOM | 2502 | OE1 | GLN | 1137 | 9.524 | 28.224 | 6.140 | 1.00 | 39.43 |
| ATOM | 2503 | NE2 | GLN | 1137 | 11.590 | 27.364 | 6.099 | 1.00 | 38.49 |
| ATOM | 2504 | HE21 | GLN | 1137 | 12.532 | 27.447 | 6.328 | 1.00 | 25.00 |
| ATOM | 2505 | HE22 | GLN | 1137 | 11.274 | 26.615 | 5.565 | 1.00 | 25.00 |
| ATOM | 2506 | C | GLN | 1137 | 11.764 | 28.971 | 10.309 | 1.00 | 34.66 |
| ATOM | 2507 | O | GLN | 1137 | 12.688 | 28.425 | 9.637 | 1.00 | 34.71 |
| ATOM | 2508 | N | THR | 1138 | 12.054 | 29.610 | 11.502 | 1.00 | 34.40 |
| ATOM | 2509 | H | THR | 1138 | 11.464 | 30.277 | 11.929 | 1.00 | 25.00 |
| ATOM | 2510 | CA | THR | 1138 | 13.459 | 29.357 | 11.986 | 1.00 | 31.73 |
| ATOM | 2511 | CB | THR | 1138 | 14.092 | 30.288 | 13.080 | 1.00 | 35.22 |
| ATOM | 2512 | OG1 | THR | 1138 | 14.053 | 31.734 | 12.891 | 1.00 | 36.89 |
| ATOM | 2513 | HG1 | THR | 1138 | 14.472 | 32.218 | 13.601 | 1.00 | 25.00 |
| ATOM | 2514 | CG2 | THR | 1138 | 15.577 | 30.011 | 12.776 | 1.00 | 35.02 |
| ATOM | 2515 | C | THR | 1138 | 13.355 | 27.983 | 12.561 | 1.00 | 27.81 |
| ATOM | 2516 | O | THR | 1138 | 14.156 | 27.153 | 12.128 | 1.00 | 25.13 |
| ATOM | 2517 | N | MET | 1139 | 12.261 | 27.681 | 13.295 | 1.00 | 23.68 |
| ATOM | 2518 | H | MET | 1139 | 11.590 | 28.369 | 13.439 | 1.00 | 25.00 |
| ATOM | 2519 | CA | MET | 1139 | 12.063 | 26.334 | 13.845 | 1.00 | 26.59 |
| ATOM | 2520 | CB | MET | 1139 | 10.700 | 26.122 | 14.515 | 1.00 | 25.38 |
| ATOM | 2521 | CG | MET | 1139 | 10.436 | 27.149 | 15.563 | 1.00 | 22.42 |
| ATOM | 2522 | SD | MET | 1139 | 8.753 | 27.056 | 16.115 | 1.00 | 26.30 |
| ATOM | 2523 | CE | MET | 1139 | 8.276 | 28.680 | 16.812 | 1.00 | 22.38 |
| ATOM | 2524 | C | MET | 1139 | 12.133 | 25.354 | 12.688 | 1.00 | 28.91 |
| ATOM | 2525 | O | MET | 1139 | 13.013 | 24.504 | 12.626 | 1.00 | 30.62 |
| ATOM | 2526 | N | LEU | 1140 | 11.310 | 25.467 | 11.640 | 1.00 | 32.59 |
| ATOM | 2527 | H | LEU | 1140 | 10.605 | 26.161 | 11.632 | 1.00 | 25.00 |
| ATOM | 2528 | CA | LEU | 1140 | 11.486 | 24.528 | 10.512 | 1.00 | 31.84 |
| ATOM | 2529 | CB | LEU | 1140 | 10.697 | 24.925 | 9.319 | 1.00 | 31.87 |
| ATOM | 2530 | CG | LEU | 1140 | 9.246 | 25.171 | 9.506 | 1.00 | 32.21 |
| ATOM | 2531 | CD1 | LEU | 1140 | 8.848 | 25.622 | 8.116 | 1.00 | 30.84 |
| ATOM | 2532 | CD2 | LEU | 1140 | 8.481 | 24.018 | 10.154 | 1.00 | 29.66 |
| ATOM | 2533 | C | LEU | 1140 | 12.928 | 24.488 | 10.011 | 1.00 | 32.79 |
| ATOM | 2534 | O | LEU | 1140 | 13.492 | 23.455 | 9.688 | 1.00 | 36.00 |
| ATOM | 2535 | N | ASP | 1141 | 13.641 | 25.568 | 9.905 | 1.00 | 34.15 |
| ATOM | 2536 | H | ASP | 1141 | 13.287 | 26.450 | 10.179 | 1.00 | 25.00 |
| ATOM | 2537 | CA | ASP | 1141 | 15.022 | 25.439 | 9.421 | 1.00 | 36.61 |
| ATOM | 2538 | CB | ASP | 1141 | 15.520 | 26.896 | 9.376 | 1.00 | 45.00 |
| ATOM | 2539 | CG | ASP | 1141 | 15.111 | 27.695 | 8.122 | 1.00 | 49.02 |
| ATOM | 2540 | OD1 | ASP | 1141 | 15.696 | 28.750 | 7.947 | 1.00 | 51.91 |
| ATOM | 2541 | OD2 | ASP | 1141 | 14.252 | 27.310 | 7.314 | 1.00 | 55.03 |
| ATOM | 2542 | C | ASP | 1141 | 15.899 | 24.520 | 10.277 | 1.00 | 35.98 |
| ATOM | 2543 | O | ASP | 1141 | 16.905 | 23.885 | 9.944 | 1.00 | 33.37 |
| ATOM | 2544 | N | CYS | 1142 | 15.548 | 24.621 | 11.544 | 1.00 | 36.95 |
| ATOM | 2545 | H | CYS | 1142 | 14.835 | 25.250 | 11.789 | 1.00 | 25.00 |
| ATOM | 2546 | CA | CYS | 1142 | 16.255 | 23.863 | 12.610 | 1.00 | 35.40 |
| ATOM | 2547 | CB | CYS | 1142 | 15.850 | 24.524 | 14.043 | 1.00 | 34.46 |
| ATOM | 2548 | SG | CYS | 1142 | 17.060 | 25.798 | 14.569 | 1.00 | 32.93 |
| ATOM | 2549 | C | CYS | 1142 | 15.987 | 22.395 | 12.501 | 1.00 | 33.96 |
| ATOM | 2550 | O | CYS | 1142 | 16.864 | 21.614 | 12.854 | 1.00 | 33.23 |
| ATOM | 2551 | N | TRP | 1143 | 14.779 | 22.083 | 11.990 | 1.00 | 33.68 |
| ATOM | 2552 | H | TRP | 1143 | 14.177 | 22.839 | 11.837 | 1.00 | 25.00 |
| ATOM | 2553 | CA | TRP | 1143 | 14.263 | 20.738 | 11.766 | 1.00 | 33.12 |
| ATOM | 2554 | CB | TRP | 1143 | 12.777 | 20.849 | 11.936 | 1.00 | 25.16 |
| ATOM | 2555 | CG | TRP | 1143 | 12.426 | 21.295 | 13.283 | 1.00 | 18.67 |
| ATOM | 2556 | CD2 | TRP | 1143 | 11.177 | 21.803 | 13.747 | 1.00 | 19.35 |
| ATOM | 2557 | CE2 | TRP | 1143 | 11.301 | 21.921 | 15.157 | 1.00 | 16.94 |
| ATOM | 2558 | CE3 | TRP | 1143 | 9.927 | 22.188 | 13.110 | 1.00 | 20.55 |
| ATOM | 2559 | CD1 | TRP | 1143 | 13.247 | 21.132 | 14.415 | 1.00 | 19.30 |
| ATOM | 2560 | NE1 | TRP | 1143 | 12.569 | 21.505 | 15.550 | 1.00 | 19.06 |
| ATOM | 2561 | HE1 | TRP | 1143 | 12.912 | 21.451 | 16.464 | 1.00 | 25.00 |
| ATOM | 2562 | CZ2 | TRP | 1143 | 10.182 | 22.401 | 15.871 | 1.00 | 18.02 |
| ATOM | 2563 | CZ3 | TRP | 1143 | 8.818 | 22.663 | 13.857 | 1.00 | 18.22 |
| ATOM | 2564 | CH2 | TRP | 1143 | 8.957 | 22.778 | 15.234 | 1.00 | 17.34 |
| ATOM | 2565 | C | TRP | 1143 | 14.660 | 20.102 | 10.446 | 1.00 | 38.77 |
| ATOM | 2566 | O | TRP | 1143 | 14.227 | 19.026 | 10.021 | 1.00 | 42.28 |
| ATOM | 2567 | N | HIS | 1144 | 15.604 | 20.709 | 9.752 | 1.00 | 42.60 |
| ATOM | 2568 | H | HIS | 1144 | 15.960 | 21.574 | 10.046 | 1.00 | 25.00 |
| ATOM | 2569 | CA | HIS | 1144 | 16.035 | 20.099 | 8.499 | 1.00 | 43.58 |
| ATOM | 2570 | C | HIS | 1144 | 16.803 | 18.736 | 8.616 | 1.00 | 44.75 |
| ATOM | 2571 | O | HIS | 1144 | 17.979 | 18.583 | 8.977 | 1.00 | 43.39 |
| ATOM | 2572 | CB | HIS | 1144 | 16.817 | 21.246 | 7.785 | 1.00 | 44.46 |
| ATOM | 2573 | CG | HIS | 1144 | 17.428 | 20.768 | 6.505 | 1.00 | 47.28 |
| ATOM | 2574 | ND1 | HIS | 1144 | 17.082 | 21.248 | 5.279 | 1.00 | 45.53 |
| ATOM | 2575 | HD1 | HIS | 1144 | 16.456 | 21.972 | 5.089 | 1.00 | 25.00 |
| ATOM | 2576 | CD2 | HIS | 1144 | 18.305 | 19.683 | 6.312 | 1.00 | 46.14 |
| ATOM | 2577 | NE2 | HIS | 1144 | 18.484 | 19.484 | 4.972 | 1.00 | 47.91 |
| ATOM | 2578 | CE1 | HIS | 1144 | 17.730 | 20.452 | 4.354 | 1.00 | 47.95 |
| ATOM | 2579 | N | GLY | 1145 | 16.123 | 17.761 | 8.018 | 1.00 | 45.34 |
| ATOM | 2580 | H | GLY | 1145 | 15.278 | 18.044 | 7.647 | 1.00 | 25.00 |
| ATOM | 2581 | CA | GLY | 1145 | 16.544 | 16.374 | 7.953 | 1.00 | 45.62 |
| ATOM | 2582 | C | GLY | 1145 | 18.010 | 16.114 | 7.924 | 1.00 | 48.92 |
| ATOM | 2583 | O | GLY | 1145 | 18.498 | 15.144 | 8.537 | 1.00 | 50.46 |
| ATOM | 2584 | N | GLU | 1146 | 18.669 | 16.791 | 7.026 | 1.00 | 50.27 |
| ATOM | 2585 | H | GLU | 1146 | 18.222 | 17.373 | 6.396 | 1.00 | 25.00 |
| ATOM | 2586 | CA | GLU | 1146 | 20.080 | 16.567 | 7.029 | 1.00 | 54.60 |
| ATOM | 2587 | CB | GLU | 1146 | 20.835 | 16.836 | 5.694 | 1.00 | 58.82 |
| ATOM | 2588 | CG | GLU | 1146 | 20.927 | 15.586 | 4.779 | 1.00 | 65.58 |
| ATOM | 2589 | CD | GLU | 1146 | 19.614 | 15.431 | 3.977 | 1.00 | 69.68 |
| ATOM | 2590 | OE1 | GLU | 1146 | 18.652 | 14.802 | 4.433 | 1.00 | 73.42 |
| ATOM | 2591 | OE2 | GLU | 1146 | 19.537 | 15.949 | 2.869 | 1.00 | 69.92 |
| ATOM | 2592 | C | GLU | 1146 | 20.623 | 17.521 | 8.028 | 1.00 | 53.76 |
| ATOM | 2593 | O | GLU | 1146 | 20.344 | 18.726 | 8.005 | 1.00 | 54.34 |
| ATOM | 2594 | N | PRO | 1147 | 21.433 | 16.948 | 8.901 | 1.00 | 53.84 |
| ATOM | 2595 | CD | PRO | 1147 | 21.586 | 15.498 | 9.134 | 1.00 | 50.10 |
| ATOM | 2596 | CA | PRO | 1147 | 22.290 | 17.742 | 9.802 | 1.00 | 54.29 |
| ATOM | 2597 | CB | PRO | 1147 | 23.322 | 16.612 | 10.149 | 1.00 | 53.82 |
| ATOM | 2598 | CG | PRO | 1147 | 23.082 | 15.480 | 9.117 | 1.00 | 51.48 |
| ATOM | 2599 | C | PRO | 1147 | 22.828 | 19.103 | 9.224 | 1.00 | 53.73 |
| ATOM | 2600 | O | PRO | 1147 | 22.417 | 20.181 | 9.587 | 1.00 | 53.88 |
| ATOM | 2601 | N | SER | 1148 | 23.746 | 19.090 | 8.264 | 1.00 | 54.72 |
| ATOM | 2602 | H | SER | 1148 | 24.082 | 18.204 | 8.070 | 1.00 | 25.00 |
| ATOM | 2603 | CA | SER | 1148 | 24.461 | 20.203 | 7.515 | 1.00 | 53.18 |
| ATOM | 2604 | CB | SER | 1148 | 25.416 | 19.660 | 6.447 | 1.00 | 56.83 |
| ATOM | 2605 | OG | SER | 1148 | 24.654 | 19.181 | 5.306 | 1.00 | 59.90 |
| ATOM | 2606 | HG | SER | 1148 | 25.289 | 18.927 | 4.627 | 1.00 | 25.00 |
| ATOM | 2607 | C | SER | 1148 | 23.716 | 21.248 | 6.742 | 1.00 | 50.57 |
| ATOM | 2608 | O | SER | 1148 | 24.209 | 21.990 | 5.886 | 1.00 | 49.52 |
| ATOM | 2609 | N | GLN | 1149 | 22.469 | 21.163 | 6.900 | 1.00 | 48.40 |
| ATOM | 2610 | H | GLN | 1149 | 22.070 | 20.518 | 7.505 | 1.00 | 25.00 |
| ATOM | 2611 | CA | GLN | 1149 | 21.651 | 22.101 | 6.227 | 1.00 | 47.20 |
| ATOM | 2612 | CB | GLN | 1149 | 20.910 | 21.165 | 5.319 | 1.00 | 49.94 |
| ATOM | 2613 | CG | GLN | 1149 | 21.700 | 20.823 | 4.047 | 1.00 | 51.72 |
| ATOM | 2614 | CD | GLN | 1149 | 21.708 | 22.109 | 3.247 | 1.00 | 52.73 |
| ATOM | 2615 | OE1 | GLN | 1149 | 20.705 | 22.603 | 2.740 | 1.00 | 57.15 |
| ATOM | 2616 | NE2 | GLN | 1149 | 22.803 | 22.778 | 3.165 | 1.00 | 52.69 |
| ATOM | 2617 | HE21 | GLN | 1149 | 23.631 | 22.497 | 3.607 | 1.00 | 25.00 |
| ATOM | 2618 | HE22 | GLN | 1149 | 22.742 | 23.591 | 2.645 | 1.00 | 25.00 |
| ATOM | 2619 | C | GLN | 1149 | 20.872 | 22.905 | 7.280 | 1.00 | 46.54 |
| ATOM | 2620 | O | GLN | 1149 | 19.980 | 23.754 | 7.058 | 1.00 | 46.14 |
| ATOM | 2621 | N | ARG | 1150 | 21.077 | 22.497 | 8.540 | 1.00 | 44.41 |
| ATOM | 2622 | H | ARG | 1150 | 21.646 | 21.732 | 8.759 | 1.00 | 25.00 |
| ATOM | 2623 | CA | ARG | 1150 | 20.438 | 23.240 | 9.634 | 1.00 | 42.55 |
| ATOM | 2624 | CB | ARG | 1150 | 20.484 | 22.391 | 10.924 | 1.00 | 40.43 |
| ATOM | 2625 | CG | ARG | 1150 | 19.663 | 21.150 | 10.796 | 1.00 | 37.13 |
| ATOM | 2626 | CD | ARG | 1150 | 19.553 | 20.522 | 12.146 | 1.00 | 37.38 |
| ATOM | 2627 | NE | ARG | 1150 | 18.772 | 19.291 | 11.874 | 1.00 | 38.63 |
| ATOM | 2628 | HE | ARG | 1150 | 17.871 | 19.390 | 11.468 | 1.00 | 25.00 |
| ATOM | 2629 | CZ | ARG | 1150 | 19.189 | 18.013 | 12.157 | 1.00 | 36.07 |
| ATOM | 2630 | NH1 | ARG | 1150 | 20.367 | 17.753 | 12.717 | 1.00 | 32.01 |
| ATOM | 2631 | HH11 | ARG | 1150 | 20.981 | 18.507 | 12.938 | 1.00 | 25.00 |
| ATOM | 2632 | HH12 | ARG | 1150 | 20.620 | 16.793 | 12.874 | 1.00 | 25.00 |
| ATOM | 2633 | NH2 | ARG | 1150 | 18.416 | 16.975 | 11.789 | 1.00 | 34.56 |
| ATOM | 2634 | HH21 | ARG | 1150 | 17.563 | 17.161 | 11.301 | 1.00 | 25.00 |
| ATOM | 2635 | HH22 | ARG | 1150 | 18.689 | 16.025 | 11.945 | 1.00 | 25.00 |
| ATOM | 2636 | C | ARG | 1150 | 21.231 | 24.592 | 9.823 | 1.00 | 43.83 |
| ATOM | 2637 | O | ARG | 1150 | 22.437 | 24.732 | 9.462 | 1.00 | 43.72 |
| ATOM | 2638 | N | PRO | 1151 | 20.569 | 25.629 | 10.428 | 1.00 | 42.67 |
| ATOM | 2639 | CD | PRO | 1151 | 19.114 | 25.757 | 10.735 | 1.00 | 41.47 |
| ATOM | 2640 | CA | PRO | 1151 | 21.347 | 26.779 | 10.954 | 1.00 | 41.79 |
| ATOM | 2641 | CB | PRO | 1151 | 20.305 | 27.587 | 11.663 | 1.00 | 42.68 |
| ATOM | 2642 | CG | PRO | 1151 | 18.964 | 27.254 | 11.013 | 1.00 | 40.68 |
| ATOM | 2643 | C | PRO | 1151 | 22.510 | 26.445 | 11.895 | 1.00 | 42.33 |
| ATOM | 2644 | O | PRO | 1151 | 22.465 | 25.437 | 12.594 | 1.00 | 42.51 |
| ATOM | 2645 | N | THR | 1152 | 23.602 | 27.171 | 11.916 | 1.00 | 40.10 |
| ATOM | 2646 | H | THR | 1152 | 23.748 | 27.920 | 11.316 | 1.00 | 25.00 |
| ATOM | 2647 | CA | THR | 1152 | 24.584 | 26.860 | 12.949 | 1.00 | 39.68 |
| ATOM | 2648 | CB | THR | 1152 | 25.983 | 27.286 | 12.613 | 1.00 | 42.74 |
| ATOM | 2649 | OG1 | THR | 1152 | 25.813 | 28.650 | 12.227 | 1.00 | 47.80 |
| ATOM | 2650 | HG1 | THR | 1152 | 26.716 | 28.981 | 12.086 | 1.00 | 25.00 |
| ATOM | 2651 | CG2 | THR | 1152 | 26.750 | 26.456 | 11.545 | 1.00 | 41.87 |
| ATOM | 2652 | C | THR | 1152 | 24.176 | 27.747 | 14.158 | 1.00 | 37.98 |
| ATOM | 2653 | O | THR | 1152 | 23.346 | 28.675 | 14.044 | 1.00 | 36.59 |
| ATOM | 2654 | N | PHE | 1153 | 24.801 | 27.572 | 15.337 | 1.00 | 36.72 |
| ATOM | 2655 | H | PHE | 1153 | 25.480 | 26.872 | 15.440 | 1.00 | 25.00 |
| ATOM | 2656 | CA | PHE | 1153 | 24.370 | 28.444 | 16.440 | 1.00 | 33.91 |
| ATOM | 2657 | CB | PHE | 1153 | 24.915 | 27.765 | 17.749 | 1.00 | 28.96 |
| ATOM | 2658 | CG | PHE | 1153 | 23.897 | 26.845 | 18.376 | 1.00 | 26.29 |
| ATOM | 2659 | CD1 | PHE | 1153 | 22.628 | 27.245 | 18.795 | 1.00 | 23.02 |
| ATOM | 2660 | CD2 | PHE | 1153 | 24.180 | 25.480 | 18.490 | 1.00 | 27.42 |
| ATOM | 2661 | CE1 | PHE | 1153 | 21.653 | 26.380 | 19.289 | 1.00 | 21.58 |
| ATOM | 2662 | CE2 | PHE | 1153 | 23.204 | 24.612 | 18.989 | 1.00 | 22.10 |
| ATOM | 2663 | CZ | PHE | 1153 | 21.965 | 25.051 | 19.381 | 1.00 | 17.48 |
| ATOM | 2664 | C | PHE | 1153 | 24.889 | 29.879 | 16.103 | 1.00 | 34.27 |
| ATOM | 2665 | O | PHE | 1153 | 24.243 | 30.871 | 16.383 | 1.00 | 29.84 |
| ATOM | 2666 | N | SER | 1154 | 26.014 | 30.041 | 15.399 | 1.00 | 35.98 |
| ATOM | 2667 | H | SER | 1154 | 26.507 | 29.232 | 15.167 | 1.00 | 25.00 |
| ATOM | 2668 | CA | SER | 1154 | 26.517 | 31.432 | 15.028 | 1.00 | 38.48 |
| ATOM | 2669 | CB | SER | 1154 | 27.893 | 31.254 | 14.380 | 1.00 | 39.87 |
| ATOM | 2670 | OG | SER | 1154 | 28.816 | 30.455 | 15.219 | 1.00 | 38.56 |
| ATOM | 2671 | HG | SER | 1154 | 29.614 | 30.251 | 14.714 | 1.00 | 25.00 |
| ATOM | 2672 | C | SER | 1154 | 25.520 | 32.211 | 14.127 | 1.00 | 38.31 |
| ATOM | 2673 | O | SER | 1154 | 25.121 | 33.343 | 14.431 | 1.00 | 40.78 |
| ATOM | 2674 | N | GLU | 1155 | 24.996 | 31.610 | 13.093 | 1.00 | 35.94 |
| ATOM | 2675 | H | GLU | 1155 | 25.280 | 30.690 | 12.923 | 1.00 | 25.00 |
| ATOM | 2676 | CA | GLU | 1155 | 23.988 | 32.274 | 12.309 | 1.00 | 32.96 |
| ATOM | 2677 | CB | GLU | 1155 | 23.626 | 31.511 | 11.177 | 1.00 | 37.45 |
| ATOM | 2678 | CG | GLU | 1155 | 24.850 | 31.023 | 10.437 | 1.00 | 43.51 |
| ATOM | 2679 | CD | GLU | 1155 | 24.399 | 29.902 | 9.504 | 1.00 | 46.96 |
| ATOM | 2680 | OE1 | GLU | 1155 | 25.310 | 29.165 | 9.071 | 1.00 | 50.41 |
| ATOM | 2681 | OE2 | GLU | 1155 | 23.174 | 29.774 | 9.233 | 1.00 | 45.70 |
| ATOM | 2682 | C | GLU | 1155 | 22.749 | 32.393 | 13.103 | 1.00 | 31.69 |
| ATOM | 2683 | O | GLU | 1155 | 21.990 | 33.275 | 12.792 | 1.00 | 31.93 |
| ATOM | 2684 | N | LEU | 1156 | 22.326 | 31.477 | 13.946 | 1.00 | 30.61 |
| ATOM | 2685 | H | LEU | 1156 | 22.751 | 30.604 | 14.002 | 1.00 | 25.00 |
| ATOM | 2686 | CA | LEU | 1156 | 21.132 | 31.740 | 14.761 | 1.00 | 29.56 |
| ATOM | 2687 | CB | LEU | 1156 | 20.789 | 30.550 | 15.503 | 1.00 | 27.75 |
| ATOM | 2688 | CG | LEU | 1156 | 20.180 | 29.409 | 14.650 | 1.00 | 26.38 |
| ATOM | 2689 | CD1 | LEU | 1156 | 20.209 | 28.026 | 15.442 | 1.00 | 23.83 |
| ATOM | 2690 | CD2 | LEU | 1156 | 18.707 | 29.666 | 14.410 | 1.00 | 19.77 |
| ATOM | 2691 | C | LEU | 1156 | 21.300 | 32.894 | 15.783 | 1.00 | 30.48 |
| ATOM | 2692 | O | LEU | 1156 | 20.353 | 33.434 | 16.353 | 1.00 | 34.15 |
| ATOM | 2693 | N | VAL | 1157 | 22.501 | 33.269 | 16.207 | 1.00 | 32.36 |
| ATOM | 2694 | H | VAL | 1157 | 23.281 | 32.770 | 15.909 | 1.00 | 25.00 |
| ATOM | 2695 | CA | VAL | 1157 | 22.702 | 34.447 | 17.096 | 1.00 | 31.69 |
| ATOM | 2696 | CB | VAL | 1157 | 24.188 | 34.541 | 17.625 | 1.00 | 31.12 |
| ATOM | 2697 | CG1 | VAL | 1157 | 24.461 | 35.828 | 18.434 | 1.00 | 29.72 |
| ATOM | 2698 | CG2 | VAL | 1157 | 24.414 | 33.474 | 18.695 | 1.00 | 26.93 |
| ATOM | 2699 | C | VAL | 1157 | 22.427 | 35.689 | 16.161 | 1.00 | 32.74 |
| ATOM | 2700 | O | VAL | 1157 | 21.539 | 36.563 | 16.409 | 1.00 | 31.94 |
| ATOM | 2701 | N | GLU | 1158 | 23.190 | 35.628 | 15.010 | 1.00 | 33.47 |
| ATOM | 2702 | H | GLU | 1158 | 23.818 | 34.854 | 14.912 | 1.00 | 25.00 |
| ATOM | 2703 | CA | GLU | 1158 | 23.128 | 36.698 | 13.942 | 1.00 | 36.69 |
| ATOM | 2704 | CB | GLU | 1158 | 23.968 | 36.147 | 12.752 | 1.00 | 39.75 |
| ATOM | 2705 | CG | GLU | 1158 | 23.927 | 36.616 | 11.273 | 1.00 | 49.25 |
| ATOM | 2706 | CD | GLU | 1158 | 25.128 | 36.142 | 10.367 | 1.00 | 52.53 |
| ATOM | 2707 | OE1 | GLU | 1158 | 25.653 | 35.026 | 10.525 | 1.00 | 56.14 |
| ATOM | 2708 | OE2 | GLU | 1158 | 25.526 | 36.909 | 9.474 | 1.00 | 51.73 |
| ATOM | 2709 | C | GLU | 1158 | 21.688 | 36.989 | 13.620 | 1.00 | 35.87 |
| ATOM | 2710 | O | GLU | 1158 | 21.085 | 38.021 | 13.992 | 1.00 | 37.92 |
| ATOM | 2711 | N | HIS | 1159 | 21.031 | 35.955 | 13.143 | 1.00 | 34.16 |
| ATOM | 2712 | H | HIS | 1159 | 21.505 | 35.115 | 13.022 | 1.00 | 25.00 |
| ATOM | 2713 | CA | HIS | 1159 | 19.648 | 36.091 | 12.765 | 1.00 | 33.52 |
| ATOM | 2714 | C | HIS | 1159 | 18.679 | 36.455 | 13.833 | 1.00 | 34.58 |
| ATOM | 2715 | O | HIS | 1159 | 17.888 | 37.407 | 13.671 | 1.00 | 36.50 |
| ATOM | 2716 | CB | HIS | 1159 | 19.270 | 34.813 | 12.068 | 1.00 | 35.94 |
| ATOM | 2717 | CG | HIS | 1159 | 17.850 | 34.751 | 11.646 | 1.00 | 38.95 |
| ATOM | 2718 | ND1 | HIS | 1159 | 16.839 | 34.072 | 12.310 | 1.00 | 41.78 |
| ATOM | 2719 | HD1 | HIS | 1159 | 16.900 | 33.563 | 13.134 | 1.00 | 25.00 |
| ATOM | 2720 | CD2 | HIS | 1159 | 17.320 | 35.405 | 10.568 | 1.00 | 37.44 |
| ATOM | 2721 | NE2 | HIS | 1159 | 16.003 | 35.077 | 10.649 | 1.00 | 40.99 |
| ATOM | 2722 | CE1 | HIS | 1159 | 15.667 | 34.274 | 11.688 | 1.00 | 39.22 |
| ATOM | 2723 | N | LEU | 1160 | 18.600 | 35.674 | 14.902 | 1.00 | 33.23 |
| ATOM | 2724 | H | LEU | 1160 | 19.255 | 34.967 | 15.063 | 1.00 | 25.00 |
| ATOM | 2725 | CA | LEU | 1160 | 17.656 | 36.064 | 15.961 | 1.00 | 31.25 |
| ATOM | 2726 | CB | LEU | 1160 | 17.789 | 35.009 | 17.043 | 1.00 | 28.95 |
| ATOM | 2727 | CG | LEU | 1160 | 16.906 | 33.877 | 16.776 | 1.00 | 27.75 |
| ATOM | 2728 | CD1 | LEU | 1160 | 17.513 | 32.761 | 17.628 | 1.00 | 28.12 |
| ATOM | 2729 | CD2 | LEU | 1160 | 15.422 | 34.234 | 17.016 | 1.00 | 25.31 |
| ATOM | 2730 | C | LEU | 1160 | 17.964 | 37.509 | 16.455 | 1.00 | 31.35 |
| ATOM | 2731 | O | LEU | 1160 | 17.013 | 38.221 | 16.766 | 1.00 | 31.81 |
| ATOM | 2732 | N | GLY | 1161 | 19.236 | 37.983 | 16.504 | 1.00 | 31.88 |
| ATOM | 2733 | H | GLY | 1161 | 19.936 | 37.353 | 16.289 | 1.00 | 25.00 |
| ATOM | 2734 | CA | GLY | 1161 | 19.654 | 39.404 | 16.858 | 1.00 | 33.53 |
| ATOM | 2735 | C | GLY | 1161 | 19.000 | 40.399 | 15.843 | 1.00 | 32.51 |
| ATOM | 2736 | O | GLY | 1161 | 18.265 | 41.318 | 16.239 | 1.00 | 32.68 |
| ATOM | 2737 | N | ASN | 1162 | 19.215 | 40.234 | 14.512 | 1.00 | 32.51 |
| ATOM | 2738 | H | ASN | 1162 | 19.789 | 39.475 | 14.234 | 1.00 | 25.00 |
| ATOM | 2739 | CA | ASN | 1162 | 18.500 | 41.110 | 13.507 | 1.00 | 33.28 |
| ATOM | 2740 | CB | ASN | 1162 | 18.514 | 40.653 | 12.073 | 1.00 | 35.11 |
| ATOM | 2741 | CG | ASN | 1162 | 19.831 | 40.177 | 11.438 | 1.00 | 39.94 |
| ATOM | 2742 | OD1 | ASN | 1162 | 20.996 | 40.519 | 11.709 | 1.00 | 40.26 |
| ATOM | 2743 | ND2 | ASN | 1162 | 19.651 | 39.315 | 10.436 | 1.00 | 44.47 |
| ATOM | 2744 | HD21 | ASN | 1162 | 18.764 | 39.022 | 10.157 | 1.00 | 25.00 |
| ATOM | 2745 | HD22 | ASN | 1162 | 20.430 | 38.960 | 9.965 | 1.00 | 25.00 |
| ATOM | 2746 | C | ASN | 1162 | 17.006 | 41.043 | 13.810 | 1.00 | 34.66 |
| ATOM | 2747 | O | ASN | 1162 | 16.248 | 42.013 | 13.740 | 1.00 | 35.08 |
| ATOM | 2748 | N | LEU | 1163 | 16.495 | 39.864 | 14.142 | 1.00 | 36.92 |
| ATOM | 2749 | H | LEU | 1163 | 17.054 | 39.067 | 14.082 | 1.00 | 25.00 |
| ATOM | 2750 | CA | LEU | 1163 | 15.082 | 39.752 | 14.484 | 1.00 | 38.43 |
| ATOM | 2751 | CB | LEU | 1163 | 14.770 | 38.292 | 14.623 | 1.00 | 39.60 |
| ATOM | 2752 | CG | LEU | 1163 | 13.909 | 37.970 | 13.369 | 1.00 | 43.41 |
| ATOM | 2753 | CD1 | LEU | 1163 | 14.877 | 37.594 | 12.257 | 1.00 | 42.19 |
| ATOM | 2754 | CD2 | LEU | 1163 | 12.839 | 36.884 | 13.652 | 1.00 | 45.22 |
| ATOM | 2755 | C | LEU | 1163 | 14.631 | 40.516 | 15.718 | 1.00 | 39.37 |
| ATOM | 2756 | O | LEU | 1163 | 13.487 | 40.926 | 15.978 | 1.00 | 37.59 |
| ATOM | 2757 | N | LEU | 1164 | 15.572 | 40.740 | 16.597 | 1.00 | 41.87 |
| ATOM | 2758 | H | LEU | 1164 | 16.468 | 40.369 | 16.447 | 1.00 | 25.00 |
| ATOM | 2759 | CA | LEU | 1164 | 15.249 | 41.440 | 17.885 | 1.00 | 42.02 |
| ATOM | 2760 | CB | LEU | 1164 | 16.307 | 40.978 | 18.916 | 1.00 | 37.06 |
| ATOM | 2761 | CG | LEU | 1164 | 16.026 | 41.240 | 20.374 | 1.00 | 31.15 |
| ATOM | 2762 | CD1 | LEU | 1164 | 14.644 | 40.956 | 20.887 | 1.00 | 25.26 |
| ATOM | 2763 | CD2 | LEU | 1164 | 17.124 | 40.412 | 20.997 | 1.00 | 31.26 |
| ATOM | 2764 | C | LEU | 1164 | 15.202 | 42.947 | 17.635 | 1.00 | 44.08 |
| ATOM | 2765 | O | LEU | 1164 | 14.453 | 43.695 | 18.290 | 1.00 | 43.41 |
| ATOM | 2766 | N | GLN | 1165 | 16.009 | 43.341 | 16.621 | 1.00 | 44.38 |
| ATOM | 2767 | H | GLN | 1165 | 16.606 | 42.676 | 16.249 | 1.00 | 25.00 |
| ATOM | 2768 | CA | GLN | 1165 | 16.077 | 44.750 | 16.120 | 1.00 | 47.22 |
| ATOM | 2769 | CB | GLN | 1165 | 17.151 | 45.038 | 15.096 | 1.00 | 43.66 |
| ATOM | 2770 | CG | GLN | 1165 | 18.500 | 45.156 | 15.672 | 1.00 | 45.54 |
| ATOM | 2771 | CD | GLN | 1165 | 19.384 | 44.642 | 14.573 | 1.00 | 47.69 |
| ATOM | 2772 | OE1 | GLN | 1165 | 19.271 | 45.094 | 13.476 | 1.00 | 51.63 |
| ATOM | 2773 | NE2 | GLN | 1165 | 20.351 | 43.767 | 14.681 | 1.00 | 48.73 |
| ATOM | 2774 | HE21 | GLN | 1165 | 20.571 | 43.355 | 15.527 | 1.00 | 25.00 |
| ATOM | 2775 | HE22 | GLN | 1165 | 20.848 | 43.516 | 13.881 | 1.00 | 25.00 |
| ATOM | 2776 | C | GLN | 1165 | 14.800 | 44.975 | 15.336 | 1.00 | 49.42 |
| ATOM | 2777 | O | GLN | 1165 | 14.068 | 45.927 | 15.539 | 1.00 | 48.42 |
| ATOM | 2778 | N | ALA | 1166 | 14.505 | 43.989 | 14.467 | 1.00 | 52.59 |
| ATOM | 2779 | H | ALA | 1166 | 15.155 | 43.285 | 14.330 | 1.00 | 25.00 |
| ATOM | 2780 | CA | ALA | 1166 | 13.303 | 44.083 | 13.686 | 1.00 | 54.52 |
| ATOM | 2781 | CB | ALA | 1166 | 13.114 | 42.736 | 12.967 | 1.00 | 55.73 |
| ATOM | 2782 | C | ALA | 1166 | 12.171 | 44.474 | 14.673 | 1.00 | 56.17 |
| ATOM | 2783 | O | ALA | 1166 | 11.315 | 45.168 | 14.139 | 1.00 | 57.69 |
| ATOM | 2784 | N | ASN | 1167 | 11.897 | 44.077 | 15.970 | 1.00 | 57.55 |
| ATOM | 2785 | H | ASN | 1167 | 12.336 | 43.316 | 16.381 | 1.00 | 25.00 |
| ATOM | 2786 | CA | ASN | 1167 | 10.747 | 44.894 | 16.586 | 1.00 | 61.20 |
| ATOM | 2787 | CB | ASN | 1167 | 10.286 | 44.841 | 18.052 | 1.00 | 56.92 |
| ATOM | 2788 | CG | ASN | 1167 | 10.376 | 43.523 | 18.647 | 1.00 | 55.45 |
| ATOM | 2789 | OD1 | ASN | 1167 | 9.421 | 43.081 | 19.264 | 1.00 | 52.04 |
| ATOM | 2790 | ND2 | ASN | 1167 | 11.526 | 42.906 | 18.533 | 1.00 | 55.32 |
| ATOM | 2791 | HD21 | ASN | 1167 | 12.286 | 43.330 | 18.079 | 1.00 | 25.00 |
| ATOM | 2792 | HD22 | ASN | 1167 | 11.636 | 42.015 | 18.923 | 1.00 | 25.00 |
| ATOM | 2793 | C | ASN | 1167 | 11.441 | 46.257 | 16.693 | 1.00 | 65.53 |
| ATOM | 2794 | O | ASN | 1167 | 12.217 | 46.464 | 17.631 | 1.00 | 65.97 |
| ATOM | 2795 | N | ALA | 1168 | 11.123 | 47.131 | 15.719 | 1.00 | 70.77 |
| ATOM | 2796 | H | ALA | 1168 | 10.503 | 46.801 | 15.041 | 1.00 | 25.00 |
| ATOM | 2797 | CA | ALA | 1168 | 11.667 | 48.504 | 15.457 | 1.00 | 75.85 |
| ATOM | 2798 | CB | ALA | 1168 | 11.801 | 49.193 | 16.909 | 1.00 | 77.04 |
| ATOM | 2799 | C | ALA | 1168 | 12.986 | 48.694 | 14.533 | 1.00 | 79.11 |
| ATOM | 2800 | O | ALA | 1168 | 14.015 | 49.296 | 14.919 | 1.00 | 79.05 |
| ATOM | 2802 | C01 | INH | 1 | 17.151 | 26.972 | 40.312 | 1.00 | 22.71 |
| ATOM | 2803 | C02 | INH | 1 | 19.307 | 26.773 | 39.701 | 1.00 | 21.43 |
| ATOM | 2804 | N03 | INH | 1 | 20.747 | 27.150 | 39.703 | 1.00 | 19.88 |
| ATOM | 2805 | C04 | INH | 1 | 18.795 | 25.474 | 39.437 | 1.00 | 21.90 |
| ATOM | 2806 | S05 | INH | 1 | 17.144 | 25.377 | 39.846 | 1.00 | 25.57 |
| ATOM | 2807 | N06 | INH | 1 | 18.337 | 27.565 | 40.189 | 1.00 | 20.58 |
| ATOM | 2808 | N07 | INH | 1 | 15.973 | 27.739 | 40.426 | 1.00 | 23.47 |
| ATOM | 2809 | H08 | INH | 1 | 16.095 | 28.713 | 40.518 | 1.00 | 20.00 |
| ATOM | 2810 | C09 | INH | 1 | 12.245 | 26.442 | 40.837 | 1.00 | 25.76 |
| ATOM | 2811 | C10 | INH | 1 | 12.695 | 27.588 | 41.522 | 1.00 | 29.62 |
| ATOM | 2812 | C11 | INH | 1 | 13.067 | 25.692 | 40.045 | 1.00 | 23.52 |
| ATOM | 2813 | C12 | INH | 1 | 14.405 | 26.130 | 39.845 | 1.00 | 19.77 |
| ATOM | 2814 | C13 | INH | 1 | 14.783 | 27.313 | 40.490 | 1.00 | 21.73 |
| ATOM | 2815 | C14 | INH | 1 | 13.994 | 28.067 | 41.341 | 1.00 | 21.36 |
| ATOM | 2816 | O15 | INH | 1 | 11.796 | 28.191 | 42.482 | 1.00 | 33.04 |
| ATOM | 2817 | C16 | INH | 1 | 12.396 | 28.964 | 43.538 | 1.00 | 36.57 |
| ATOM | 2818 | O17 | INH | 1 | 10.927 | 25.968 | 41.005 | 1.00 | 31.21 |
| ATOM | 2819 | C18 | INH | 1 | 10.595 | 25.102 | 42.094 | 1.00 | 33.21 |
| ATOM | 2820 | O19 | INH | 1 | 12.510 | 24.556 | 39.346 | 1.00 | 20.43 |
| ATOM | 2821 | C20 | INH | 1 | 13.536 | 23.806 | 38.628 | 1.00 | 21.64 |
| ATOM | 2822 | S21 | INH | 1 | 18.921 | 23.101 | 37.905 | 1.00 | 22.21 |
| ATOM | 2823 | C22 | INH | 1 | 20.445 | 22.474 | 37.533 | 1.00 | 14.62 |
| ATOM | 2824 | C23 | INH | 1 | 21.461 | 23.244 | 38.101 | 1.00 | 9.96 |
| ATOM | 2825 | N24 | INH | 1 | 21.023 | 24.312 | 38.749 | 1.00 | 16.26 |
| ATOM | 2826 | C25 | INH | 1 | 19.631 | 24.400 | 38.738 | 1.00 | 22.61 |
| ATOM | 2827 | H26 | INH | 1 | 21.428 | 26.468 | 39.493 | 1.00 | 20.00 |
| ATOM | 2828 | H27 | INH | 1 | 21.059 | 28.062 | 39.918 | 1.00 | 20.00 |
| ATOM | 2829 | C28 | INH | 1 | 22.854 | 22.770 | 38.188 | 1.00 | 6.00 |
| ATOM | 2830 | C29 | INH | 1 | 23.554 | 22.960 | 39.319 | 1.00 | 7.53 |
| ATOM | 2831 | C30 | INH | 1 | 24.960 | 22.693 | 39.322 | 1.00 | 9.59 |
| ATOM | 2832 | C31 | INH | 1 | 25.620 | 22.233 | 38.165 | 1.00 | 9.41 |
| ATOM | 2833 | C32 | INH | 1 | 24.809 | 22.066 | 37.016 | 1.00 | 10.26 |
| ATOM | 2834 | C33 | INH | 1 | 23.460 | 22.293 | 37.060 | 1.00 | 6.27 |
| ATOM | 2835 | N34 | INH | 1 | 25.356 | 21.687 | 35.834 | 1.00 | 16.25 |
| ATOM | 2836 | C35 | INH | 1 | 25.020 | 22.180 | 34.628 | 1.00 | 18.70 |
| ATOM | 2837 | C36 | INH | 1 | 25.703 | 21.672 | 33.526 | 1.00 | 22.75 |
| ATOM | 2838 | O37 | INH | 1 | 24.122 | 22.990 | 34.473 | 1.00 | 21.96 |
| ATOM | 2839 | S38 | INH | 1 | 26.621 | 20.256 | 33.532 | 1.00 | 24.41 |
| ATOM | 2840 | C39 | INH | 1 | 27.266 | 20.562 | 32.021 | 1.00 | 18.53 |
| ATOM | 2841 | N40 | INH | 1 | 26.820 | 21.677 | 31.428 | 1.00 | 23.87 |
| ATOM | 2842 | C41 | INH | 1 | 25.934 | 22.383 | 32.227 | 1.00 | 27.04 |
| ATOM | 2843 | C43 | INH | 1 | 28.223 | 19.731 | 31.543 | 1.00 | 15.38 |
| ATOM | 2844 | C45 | INH | 1 | 25.317 | 23.634 | 31.575 | 1.00 | 23.30 |
| ATOM | 2845 | H42 | INH | 1 | 26.124 | 21.069 | 35.894 | 1.00 | 20.00 |
| ATOM | 2846 | H44 | INH | 1 | 27.102 | 22.016 | 30.556 | 1.00 | 20.00 |
| ATOM | 2847 | OH2 | WAT | 2 | 27.966 | 38.271 | 26.224 | 1.00 | 50.18 |
| ATOM | 2850 | OH2 | WAT | 3 | 28.856 | 36.562 | 28.753 | 1.00 | 36.62 |
| ATOM | 2853 | OH2 | WAT | 4 | 29.078 | 38.519 | 31.104 | 1.00 | 37.39 |
| ATOM | 2856 | OH2 | WAT | 5 | 23.273 | 37.357 | 34.794 | 1.00 | 33.83 |
| ATOM | 2859 | OH2 | WAT | 6 | 24.406 | 36.211 | 37.654 | 1.00 | 30.57 |
| ATOM | 2862 | OH2 | WAT | 7 | 21.978 | 35.767 | 41.203 | 1.00 | 37.54 |
| ATOM | 2865 | OH2 | WAT | 8 | 23.964 | 38.874 | 47.741 | 1.00 | 35.11 |
| ATOM | 2868 | OH2 | WAT | 9 | 22.923 | 39.472 | 51.359 | 1.00 | 36.64 |
| ATOM | 2871 | OH2 | WAT | 10 | 20.306 | 36.349 | 50.634 | 1.00 | 35.84 |
| ATOM | 2874 | OH2 | WAT | 11 | 18.982 | 28.226 | 53.618 | 1.00 | 37.76 |
| ATOM | 2877 | OH2 | WAT | 12 | 17.377 | 28.974 | 51.631 | 1.00 | 37.48 |
| ATOM | 2880 | OH2 | WAT | 13 | 14.942 | 27.238 | 51.083 | 1.00 | 41.19 |
| ATOM | 2883 | OH2 | WAT | 14 | 17.577 | 26.045 | 55.498 | 1.00 | 34.29 |
| ATOM | 2886 | OH2 | WAT | 15 | 36.108 | 27.236 | 57.172 | 1.00 | 53.58 |
| ATOM | 2889 | OH2 | WAT | 16 | 38.382 | 27.243 | 55.334 | 1.00 | 52.34 |
| ATOM | 2892 | OH2 | WAT | 17 | 34.636 | 22.251 | 51.437 | 1.00 | 29.32 |
| ATOM | 2895 | OH2 | WAT | 18 | 34.038 | 14.843 | 47.808 | 1.00 | 34.70 |
| ATOM | 2898 | OH2 | WAT | 19 | 26.991 | 14.911 | 46.063 | 1.00 | 27.30 |
| ATOM | 2901 | OH2 | WAT | 20 | 18.754 | 17.171 | 32.123 | 1.00 | 36.20 |
| ATOM | 2904 | OH2 | WAT | 21 | 19.251 | 14.538 | 31.049 | 1.00 | 45.30 |
| ATOM | 2907 | OH2 | WAT | 22 | 14.024 | 17.517 | 38.625 | 1.00 | 41.78 |
| ATOM | 2910 | OH2 | WAT | 23 | 9.849 | 21.389 | 35.429 | 1.00 | 48.32 |
| ATOM | 2913 | OH2 | WAT | 24 | 9.860 | 25.939 | 38.285 | 1.00 | 29.88 |
| ATOM | 2916 | OH2 | WAT | 25 | 3.634 | 26.658 | 35.234 | 1.00 | 33.33 |
| ATOM | 2919 | OH2 | WAT | 26 | 8.689 | 20.356 | 29.885 | 1.00 | 29.44 |
| ATOM | 2922 | OH2 | WAT | 27 | 6.500 | 21.783 | 29.005 | 1.00 | 32.35 |
| ATOM | 2925 | OH2 | WAT | 28 | 10.864 | 21.594 | 28.632 | 1.00 | 34.00 |
| ATOM | 2928 | OH2 | WAT | 29 | 0.881 | 20.324 | 23.776 | 1.00 | 44.70 |
| ATOM | 2931 | OH2 | WAT | 30 | 0.260 | 22.146 | 27.399 | 1.00 | 31.57 |
| ATOM | 2934 | OH2 | WAT | 31 | −0.963 | 24.683 | 24.000 | 1.00 | 36.16 |
| ATOM | 2937 | OH2 | WAT | 32 | 0.231 | 27.057 | 22.905 | 1.00 | 39.97 |
| ATOM | 2940 | OH2 | WAT | 33 | 13.321 | 38.277 | 35.165 | 1.00 | 36.99 |
| ATOM | 2943 | OH2 | WAT | 34 | 13.831 | 40.752 | 37.922 | 1.00 | 38.20 |
| ATOM | 2946 | OH2 | WAT | 35 | 13.008 | 42.215 | 27.076 | 1.00 | 27.46 |
| ATOM | 2949 | OH2 | WAT | 36 | 30.995 | 32.551 | 21.401 | 1.00 | 30.39 |
| ATOM | 2952 | OH2 | WAT | 37 | 27.563 | 34.735 | 14.899 | 1.00 | 38.01 |
| ATOM | 2955 | OH2 | WAT | 38 | 26.409 | 38.349 | 14.046 | 1.00 | 32.17 |
| ATOM | 2958 | OH2 | WAT | 39 | 23.172 | 40.263 | 13.330 | 1.00 | 32.58 |
| ATOM | 2961 | OH2 | WAT | 40 | 27.296 | 37.643 | 7.531 | 1.00 | 42.86 |
| ATOM | 2964 | OH2 | WAT | 41 | 10.515 | 40.627 | 15.078 | 1.00 | 54.13 |
| ATOM | 2967 | OH2 | WAT | 42 | 6.935 | 42.617 | 21.482 | 1.00 | 39.20 |
| ATOM | 2970 | OH2 | WAT | 43 | 2.704 | 19.829 | 10.061 | 1.00 | 42.70 |
| ATOM | 2973 | OH2 | WAT | 44 | 2.910 | 17.439 | 14.108 | 1.00 | 50.97 |
| ATOM | 2976 | OH2 | WAT | 45 | 12.227 | 13.957 | 17.403 | 1.00 | 42.17 |
| ATOM | 2979 | OH2 | WAT | 46 | 19.976 | 19.003 | 22.470 | 1.00 | 32.21 |
| ATOM | 2982 | OH2 | WAT | 47 | 18.425 | 16.374 | 22.408 | 1.00 | 44.27 |
| ATOM | 2985 | OH2 | WAT | 48 | 16.351 | 16.989 | 24.174 | 1.00 | 32.08 |
| ATOM | 2988 | OH2 | WAT | 49 | 29.280 | 28.865 | 11.379 | 1.00 | 31.84 |
| ATOM | 2991 | OH2 | WAT | 50 | 3.845 | 8.241 | 24.677 | 1.00 | 47.02 |
| ATOM | 2994 | OH2 | WAT | 51 | 2.725 | 7.689 | 16.533 | 1.00 | 44.34 |
| ATOM | 2997 | OH2 | WAT | 52 | 31.693 | 30.318 | 36.660 | 1.00 | 40.72 |
| ATOM | 3000 | OH2 | WAT | 53 | 29.539 | 28.211 | 34.664 | 1.00 | 39.56 |
| ATOM | 3003 | OH2 | WAT | 54 | 28.825 | 30.408 | 32.733 | 1.00 | 32.41 |
| ATOM | 3006 | OH2 | WAT | 55 | 35.936 | 32.734 | 32.431 | 1.00 | 58.04 |
| ATOM | 3009 | OH2 | WAT | 56 | 14.384 | 8.387 | 38.239 | 1.00 | 43.35 |
| ATOM | 3012 | OH2 | WAT | 57 | 39.167 | 22.075 | 34.568 | 1.00 | 32.92 |
| ATOM | 3015 | OH2 | WAT | 58 | 34.260 | 29.516 | 45.225 | 1.00 | 37.65 |
| ATOM | 3018 | OH2 | WAT | 59 | 35.548 | 31.839 | 43.164 | 1.00 | 44.19 |
| ATOM | 3021 | OH2 | WAT | 60 | 18.751 | 14.924 | 19.508 | 1.00 | 49.70 |
| ATOM | 3024 | OH2 | WAT | 61 | 17.328 | 15.609 | 34.116 | 1.00 | 32.21 |
| ATOM | 3027 | OH2 | WAT | 62 | 22.450 | 15.060 | 28.894 | 1.00 | 36.17 |
| ATOM | 3030 | OH2 | WAT | 63 | 26.253 | 15.965 | 27.429 | 1.00 | 38.78 |
| ATOM | 3033 | OH2 | WAT | 64 | 26.509 | 21.379 | 28.735 | 1.00 | 32.46 |
| ATOM | 3036 | OH2 | WAT | 65 | 28.092 | 19.813 | 27.451 | 1.00 | 34.48 |
| ATOM | 3039 | OH2 | WAT | 66 | 29.164 | 17.735 | 29.558 | 1.00 | 36.91 |
| ATOM | 3042 | OH2 | WAT | 67 | 25.664 | 16.483 | 33.243 | 1.00 | 42.67 |
| ATOM | 3045 | OH2 | WAT | 68 | 24.941 | 18.926 | 29.116 | 1.00 | 28.51 |
| ATOM | 3048 | OH2 | WAT | 69 | 34.994 | 20.636 | 26.303 | 1.00 | 43.54 |
| ATOM | 3051 | OH2 | WAT | 70 | 40.602 | 24.336 | 33.677 | 1.00 | 42.86 |
| ATOM | 3054 | OH2 | WAT | 71 | 40.597 | 27.093 | 36.147 | 1.00 | 43.89 |
| ATOM | 3057 | OH2 | WAT | 72 | 32.779 | 32.341 | 42.719 | 1.00 | 38.08 |
| ATOM | 3060 | OH2 | WAT | 73 | 27.925 | 38.396 | 34.138 | 1.00 | 46.14 |
| ATOM | 3063 | OH2 | WAT | 74 | 21.817 | 37.396 | 37.399 | 1.00 | 41.12 |
| ATOM | 3066 | OH2 | WAT | 75 | 20.919 | 33.985 | 39.300 | 1.00 | 35.31 |
| ATOM | 3069 | OH2 | WAT | 76 | 17.914 | 34.673 | 39.324 | 1.00 | 41.00 |
| ATOM | 3072 | OH2 | WAT | 77 | 8.919 | 39.949 | 32.093 | 1.00 | 47.82 |
| ATOM | 3075 | OH2 | WAT | 78 | 20.998 | 6.902 | 7.966 | 1.00 | 38.15 |
| ATOM | 3078 | OH2 | WAT | 79 | 20.826 | 2.603 | 9.908 | 1.00 | 33.85 |
| ATOM | 3081 | OH2 | WAT | 80 | 10.488 | 3.590 | 13.531 | 1.00 | 49.48 |
| ATOM | 3084 | OH2 | WAT | 81 | 7.992 | 2.065 | 11.817 | 1.00 | 42.13 |
| ATOM | 3087 | OH2 | WAT | 82 | 1.638 | 3.175 | 16.932 | 1.00 | 42.93 |
| ATOM | 3090 | OH2 | WAT | 83 | 10.071 | 12.935 | 24.586 | 1.00 | 36.64 |
| ATOM | 3093 | OH2 | WAT | 84 | 7.441 | 22.879 | 26.929 | 1.00 | 32.29 |
| ATOM | 3096 | OH2 | WAT | 85 | 8.144 | 21.854 | 32.161 | 1.00 | 36.88 |
| ATOM | 3099 | OH2 | WAT | 86 | 6.824 | 46.218 | 22.293 | 1.00 | 39.60 |
| ATOM | 3102 | OH2 | WAT | 87 | 26.700 | 13.448 | 34.816 | 1.00 | 40.37 |
| ATOM | 3105 | OH2 | WAT | 88 | 37.159 | 13.710 | 35.996 | 1.00 | 43.43 |
| ATOM | 3108 | OH2 | WAT | 89 | 35.932 | 12.237 | 38.828 | 1.00 | 39.31 |
| ATOM | 3111 | OH2 | WAT | 90 | 33.865 | 9.983 | 35.467 | 1.00 | 39.72 |
| ATOM | 3114 | OH2 | WAT | 91 | 45.889 | 17.101 | 32.564 | 1.00 | 43.03 |
| ATOM | 3117 | OH2 | WAT | 92 | 36.436 | 30.624 | 29.203 | 1.00 | 42.87 |
| ATOM | 3120 | OH2 | WAT | 93 | 32.251 | 34.336 | 23.718 | 1.00 | 46.02 |
| ATOM | 3123 | OH2 | WAT | 94 | 12.385 | 30.105 | 46.682 | 1.00 | 40.25 |
| ATOM | 3126 | OH2 | WAT | 95 | 29.296 | 12.883 | 47.074 | 1.00 | 38.02 |
| ATOM | 3129 | OH2 | WAT | 96 | 32.476 | 10.443 | 48.316 | 1.00 | 36.84 |
| ATOM | 3132 | OH2 | WAT | 97 | 20.768 | 35.478 | 8.527 | 1.00 | 40.94 |
| ATOM | 3135 | OH2 | WAT | 98 | 37.510 | 22.922 | 24.948 | 1.00 | 38.30 |

TABLE 4
Atomic Coordinates for
VEGFR2KD: 4-Fluoro-3-[5-(2-methoxy-pyridin-4-ylamino)-
2H-pyrazol-3-ylmethoxy]-N-(3-N-Imidazole-3-trifluoromethyl-
phenyl)-benzamide (Compound 4)
Complex Crystalline Structure

| | | | | | | | | | |
|---|---|---|---|---|---|---|---|---|---|
| ATOM | 1 | CB | GLU | 815 | 34.800 | 16.822 | 26.764 | 1.00 | 46.59 |
| ATOM | 2 | CG | GLU | 815 | 33.498 | 17.663 | 27.328 | 1.00 | 46.62 |
| ATOM | 3 | CD | GLU | 815 | 32.035 | 17.698 | 26.660 | 1.00 | 46.58 |
| ATOM | 4 | OE1 | GLU | 815 | 31.068 | 18.129 | 27.336 | 1.00 | 46.09 |
| ATOM | 5 | OE2 | GLU | 815 | 31.865 | 17.349 | 25.486 | 1.00 | 40.45 |
| ATOM | 6 | C | GLU | 815 | 36.568 | 14.917 | 27.768 | 1.00 | 47.33 |
| ATOM | 7 | O | GLU | 815 | 37.377 | 15.815 | 28.061 | 1.00 | 47.26 |
| ATOM | 8 | HT1 | GLU | 815 | 34.080 | 15.224 | 28.891 | 1.00 | 0.00 |
| ATOM | 9 | HT2 | GLU | 815 | 33.837 | 13.833 | 27.917 | 1.00 | 0.00 |
| ATOM | 10 | N | GLU | 815 | 33.958 | 14.862 | 27.921 | 1.00 | 42.60 |
| ATOM | 11 | HT3 | GLU | 815 | 33.114 | 15.308 | 27.499 | 1.00 | 0.00 |
| ATOM | 12 | CA | GLU | 815 | 35.121 | 15.255 | 27.099 | 1.00 | 45.92 |
| ATOM | 13 | N | HIS | 816 | 36.891 | 13.585 | 28.026 | 1.00 | 46.72 |
| ATOM | 14 | H | HIS | 816 | 36.360 | 12.867 | 27.630 | 1.00 | 0.00 |
| ATOM | 15 | CA | HIS | 816 | 38.151 | 13.047 | 28.690 | 1.00 | 45.51 |
| ATOM | 16 | C | HIS | 816 | 38.665 | 13.928 | 29.882 | 1.00 | 43.64 |
| ATOM | 17 | O | HIS | 816 | 39.870 | 14.161 | 30.161 | 1.00 | 40.60 |
| ATOM | 18 | CB | HIS | 816 | 39.317 | 12.826 | 27.625 | 1.00 | 49.50 |
| ATOM | 19 | CG | HIS | 816 | 39.360 | 11.490 | 26.916 | 1.00 | 52.54 |
| ATOM | 20 | ND1 | HIS | 816 | 38.377 | 11.005 | 26.066 | 1.00 | 55.10 |
| ATOM | 21 | CE1 | HIS | 816 | 38.752 | 9.740 | 25.628 | 1.00 | 53.61 |
| ATOM | 22 | CD2 | HIS | 816 | 40.369 | 10.483 | 26.989 | 1.00 | 53.73 |
| ATOM | 23 | NE2 | HIS | 816 | 39.969 | 9.426 | 26.195 | 1.00 | 53.47 |
| ATOM | 24 | HE2 | HIS | 816 | 40.434 | 8.590 | 25.970 | 1.00 | 0.00 |
| ATOM | 25 | N | CYS | 817 | 37.608 | 14.241 | 30.723 | 1.00 | 41.15 |
| ATOM | 26 | H | CYS | 817 | 36.765 | 13.790 | 30.514 | 1.00 | 0.00 |
| ATOM | 27 | CA | CYS | 817 | 37.714 | 15.132 | 31.953 | 1.00 | 38.77 |
| ATOM | 28 | CB | CYS | 817 | 36.370 | 15.449 | 32.643 | 1.00 | 36.85 |
| ATOM | 29 | SG | CYS | 817 | 35.510 | 16.168 | 31.344 | 1.00 | 38.08 |
| ATOM | 30 | C | CYS | 817 | 38.578 | 14.761 | 33.124 | 1.00 | 36.17 |
| ATOM | 31 | O | CYS | 817 | 38.978 | 15.650 | 33.848 | 1.00 | 34.59 |
| ATOM | 32 | N | GLU | 818 | 38.851 | 13.483 | 33.379 | 1.00 | 34.64 |
| ATOM | 33 | H | GLU | 818 | 38.426 | 12.860 | 32.771 | 1.00 | 0.00 |
| ATOM | 34 | CA | GLU | 818 | 39.697 | 13.055 | 34.548 | 1.00 | 32.96 |
| ATOM | 35 | CB | GLU | 818 | 39.809 | 11.541 | 34.587 | 1.00 | 30.96 |
| ATOM | 36 | CG | GLU | 818 | 38.455 | 10.849 | 34.439 | 1.00 | 30.36 |
| ATOM | 37 | CD | GLU | 818 | 37.881 | 10.805 | 33.001 | 1.00 | 31.72 |
| ATOM | 38 | OE1 | GLU | 818 | 36.687 | 10.496 | 32.914 | 1.00 | 35.26 |
| ATOM | 39 | OE2 | GLU | 818 | 38.559 | 11.041 | 31.989 | 1.00 | 33.10 |
| ATOM | 40 | C | GLU | 818 | 41.085 | 13.646 | 34.521 | 1.00 | 33.17 |
| ATOM | 41 | O | GLU | 818 | 41.724 | 13.842 | 35.556 | 1.00 | 34.36 |
| ATOM | 42 | N | ARG | 819 | 41.565 | 13.911 | 33.287 | 1.00 | 32.94 |
| ATOM | 43 | H | ARG | 819 | 41.006 | 13.667 | 32.526 | 1.00 | 0.00 |
| ATOM | 44 | CA | ARG | 819 | 42.927 | 14.510 | 33.036 | 1.00 | 34.53 |
| ATOM | 45 | CB | ARG | 819 | 43.275 | 14.253 | 31.548 | 1.00 | 35.22 |
| ATOM | 46 | C | ARG | 819 | 43.065 | 16.061 | 33.346 | 1.00 | 35.31 |
| ATOM | 47 | O | ARG | 819 | 44.142 | 16.685 | 33.496 | 1.00 | 32.77 |
| ATOM | 48 | N | LEU | 820 | 41.856 | 16.687 | 33.318 | 1.00 | 34.63 |
| ATOM | 49 | H | LEU | 820 | 41.083 | 16.103 | 33.303 | 1.00 | 0.00 |
| ATOM | 50 | CA | LEU | 820 | 41.640 | 18.138 | 33.530 | 1.00 | 33.39 |
| ATOM | 51 | CB | LEU | 820 | 40.092 | 18.401 | 33.217 | 1.00 | 30.45 |
| ATOM | 52 | CG | LEU | 820 | 39.572 | 19.295 | 31.983 | 1.00 | 28.57 |
| ATOM | 53 | CD1 | LEU | 820 | 40.613 | 19.194 | 30.895 | 1.00 | 26.55 |
| ATOM | 54 | CD2 | LEU | 820 | 38.181 | 18.889 | 31.450 | 1.00 | 24.01 |
| ATOM | 55 | C | LEU | 820 | 42.102 | 18.553 | 34.948 | 1.00 | 32.30 |
| ATOM | 56 | O | LEU | 820 | 42.044 | 17.787 | 35.911 | 1.00 | 31.15 |
| ATOM | 57 | N | PRO | 821 | 42.638 | 19.778 | 35.096 | 1.00 | 33.12 |
| ATOM | 58 | CD | PRO | 821 | 43.020 | 20.776 | 34.023 | 1.00 | 33.16 |
| ATOM | 59 | CA | PRO | 821 | 43.140 | 20.189 | 36.417 | 1.00 | 32.03 |
| ATOM | 60 | CB | PRO | 821 | 44.282 | 21.214 | 36.048 | 1.00 | 32.33 |
| ATOM | 61 | CG | PRO | 821 | 43.720 | 21.936 | 34.814 | 1.00 | 29.90 |
| ATOM | 62 | C | PRO | 821 | 42.086 | 20.767 | 37.412 | 1.00 | 30.65 |
| ATOM | 63 | O | PRO | 821 | 41.023 | 21.322 | 37.053 | 1.00 | 29.94 |
| ATOM | 64 | N | TYR | 822 | 42.483 | 20.685 | 38.687 | 1.00 | 27.33 |
| ATOM | 65 | H | TYR | 822 | 43.292 | 20.177 | 38.904 | 1.00 | 0.00 |
| ATOM | 66 | CA | TYR | 822 | 41.721 | 21.240 | 39.785 | 1.00 | 24.81 |
| ATOM | 67 | CB | TYR | 822 | 41.081 | 20.226 | 40.793 | 1.00 | 25.41 |
| ATOM | 68 | CG | TYR | 822 | 40.384 | 21.021 | 41.869 | 1.00 | 22.51 |
| ATOM | 69 | CD1 | TYR | 822 | 40.679 | 20.883 | 43.227 | 1.00 | 18.07 |
| ATOM | 70 | CE1 | TYR | 822 | 39.959 | 21.650 | 44.161 | 1.00 | 19.24 |
| ATOM | 71 | CD2 | TYR | 822 | 39.379 | 21.932 | 41.478 | 1.00 | 23.06 |
| ATOM | 72 | CE2 | TYR | 822 | 38.646 | 22.696 | 42.388 | 1.00 | 18.76 |
| ATOM | 73 | CZ | TYR | 822 | 38.938 | 22.549 | 43.726 | 1.00 | 20.36 |
| ATOM | 74 | OH | TYR | 822 | 38.119 | 23.236 | 44.593 | 1.00 | 19.65 |
| ATOM | 75 | HH | TYR | 822 | 38.414 | 23.127 | 45.491 | 1.00 | 0.00 |
| ATOM | 76 | C | TYR | 822 | 42.658 | 22.105 | 40.622 | 1.00 | 25.10 |
| ATOM | 77 | O | TYR | 822 | 43.446 | 21.653 | 41.459 | 1.00 | 24.32 |
| ATOM | 78 | N | ASP | 823 | 42.575 | 23.426 | 40.392 | 1.00 | 24.75 |
| ATOM | 79 | H | ASP | 823 | 41.882 | 23.779 | 39.805 | 1.00 | 0.00 |
| ATOM | 80 | CA | ASP | 823 | 43.378 | 24.409 | 41.161 | 1.00 | 22.17 |
| ATOM | 81 | CB | ASP | 823 | 43.642 | 25.657 | 40.305 | 1.00 | 22.37 |
| ATOM | 82 | CG | ASP | 823 | 44.491 | 26.633 | 41.053 | 1.00 | 22.47 |
| ATOM | 83 | OD1 | ASP | 823 | 44.944 | 26.338 | 42.155 | 1.00 | 24.53 |
| ATOM | 84 | OD2 | ASP | 823 | 44.730 | 27.694 | 40.503 | 1.00 | 25.99 |
| ATOM | 85 | C | ASP | 823 | 42.633 | 24.828 | 42.431 | 1.00 | 20.89 |
| ATOM | 86 | O | ASP | 823 | 41.880 | 25.821 | 42.492 | 1.00 | 19.33 |
| ATOM | 87 | N | ALA | 824 | 42.869 | 24.048 | 43.466 | 1.00 | 20.04 |
| ATOM | 88 | H | ALA | 824 | 43.422 | 23.254 | 43.287 | 1.00 | 0.00 |
| ATOM | 89 | CA | ALA | 824 | 42.232 | 24.285 | 44.774 | 1.00 | 21.83 |
| ATOM | 90 | CB | ALA | 824 | 42.715 | 23.253 | 45.773 | 1.00 | 20.57 |
| ATOM | 91 | C | ALA | 824 | 42.463 | 25.658 | 45.377 | 1.00 | 21.82 |
| ATOM | 92 | O | ALA | 824 | 41.643 | 26.236 | 46.090 | 1.00 | 20.41 |
| ATOM | 93 | N | SER | 825 | 43.647 | 26.194 | 45.079 | 1.00 | 26.56 |
| ATOM | 94 | H | SER | 825 | 44.301 | 25.702 | 44.556 | 1.00 | 0.00 |
| ATOM | 95 | CA | SER | 825 | 43.958 | 27.545 | 45.618 | 1.00 | 28.39 |
| ATOM | 96 | CB | SER | 825 | 45.487 | 27.985 | 45.374 | 1.00 | 28.50 |
| ATOM | 97 | OG | SER | 825 | 45.995 | 28.035 | 44.039 | 1.00 | 33.76 |
| ATOM | 98 | HG | SER | 825 | 46.932 | 28.276 | 44.069 | 1.00 | 0.00 |
| ATOM | 99 | C | SER | 825 | 43.005 | 28.541 | 45.010 | 1.00 | 25.80 |
| ATOM | 100 | O | SER | 825 | 42.664 | 29.562 | 45.605 | 1.00 | 27.55 |
| ATOM | 101 | N | LYS | 826 | 42.539 | 28.199 | 43.815 | 1.00 | 23.23 |
| ATOM | 102 | H | LYS | 826 | 42.822 | 27.377 | 43.372 | 1.00 | 0.00 |
| ATOM | 103 | CA | LYS | 826 | 41.623 | 29.098 | 43.217 | 1.00 | 19.59 |
| ATOM | 104 | CB | LYS | 826 | 41.735 | 29.092 | 41.697 | 1.00 | 17.73 |
| ATOM | 105 | CG | LYS | 826 | 40.851 | 30.177 | 41.097 | 1.00 | 13.54 |
| ATOM | 106 | CD | LYS | 826 | 41.285 | 30.407 | 39.654 | 1.00 | 16.36 |
| ATOM | 107 | CE | LYS | 826 | 40.198 | 31.223 | 38.885 | 1.00 | 19.53 |
| ATOM | 108 | NZ | LYS | 826 | 40.624 | 31.236 | 37.497 | 1.00 | 21.10 |
| ATOM | 109 | HZ1 | LYS | 826 | 40.930 | 30.294 | 37.179 | 1.00 | 0.00 |
| ATOM | 110 | HZ2 | LYS | 826 | 41.404 | 31.918 | 37.398 | 1.00 | 0.00 |
| ATOM | 111 | HZ3 | LYS | 826 | 39.820 | 31.544 | 36.916 | 1.00 | 0.00 |
| ATOM | 112 | C | LYS | 826 | 40.242 | 28.760 | 43.573 | 1.00 | 18.66 |
| ATOM | 113 | O | LYS | 826 | 39.488 | 29.619 | 43.979 | 1.00 | 20.62 |
| ATOM | 114 | N | TRP | 827 | 39.875 | 27.517 | 43.479 | 1.00 | 17.57 |
| ATOM | 115 | H | TRP | 827 | 40.525 | 26.828 | 43.265 | 1.00 | 0.00 |
| ATOM | 116 | CA | TRP | 827 | 38.437 | 27.235 | 43.699 | 1.00 | 16.27 |
| ATOM | 117 | CB | TRP | 827 | 37.972 | 26.192 | 42.678 | 1.00 | 13.24 |
| ATOM | 118 | CG | TRP | 827 | 38.279 | 26.740 | 41.386 | 1.00 | 12.06 |
| ATOM | 119 | CD2 | TRP | 827 | 37.448 | 27.671 | 40.719 | 1.00 | 13.59 |
| ATOM | 120 | CE2 | TRP | 827 | 38.015 | 27.872 | 39.433 | 1.00 | 13.90 |
| ATOM | 121 | CE3 | TRP | 827 | 36.293 | 28.338 | 41.067 | 1.00 | 11.06 |
| ATOM | 122 | CD1 | TRP | 827 | 39.360 | 26.414 | 40.509 | 1.00 | 14.46 |
| ATOM | 123 | NE1 | TRP | 827 | 39.194 | 27.118 | 39.327 | 1.00 | 12.29 |
| ATOM | 124 | HE1 | TRP | 827 | 39.804 | 27.329 | 38.580 | 1.00 | 0.00 |
| ATOM | 125 | CZ2 | TRP | 827 | 37.391 | 28.745 | 38.507 | 1.00 | 12.54 |
| ATOM | 126 | CZ3 | TRP | 827 | 35.717 | 29.194 | 40.140 | 1.00 | 13.63 |
| ATOM | 127 | CH2 | TRP | 827 | 36.238 | 29.421 | 38.866 | 1.00 | 14.18 |
| ATOM | 128 | C | TRP | 827 | 37.868 | 26.809 | 45.016 | 1.00 | 18.55 |
| ATOM | 129 | O | TRP | 827 | 36.646 | 26.943 | 45.243 | 1.00 | 16.68 |
| ATOM | 130 | N | GLU | 828 | 38.739 | 26.219 | 45.860 | 1.00 | 17.02 |
| ATOM | 131 | H | GLU | 828 | 39.703 | 26.300 | 45.707 | 1.00 | 0.00 |
| ATOM | 132 | CA | GLU | 828 | 38.194 | 25.677 | 47.112 | 1.00 | 17.90 |
| ATOM | 133 | CB | GLU | 828 | 39.388 | 24.955 | 47.746 | 1.00 | 18.43 |
| ATOM | 134 | CG | GLU | 828 | 39.039 | 24.020 | 48.920 | 1.00 | 17.69 |
| ATOM | 135 | CD | GLU | 828 | 38.368 | 22.754 | 48.466 | 1.00 | 21.44 |
| ATOM | 136 | OE1 | GLU | 828 | 37.665 | 22.214 | 49.296 | 1.00 | 22.61 |
| ATOM | 137 | OE2 | GLU | 828 | 38.558 | 22.294 | 47.342 | 1.00 | 22.13 |
| ATOM | 138 | C | GLU | 828 | 37.496 | 26.645 | 48.079 | 1.00 | 18.19 |
| ATOM | 139 | O | GLU | 828 | 37.993 | 27.676 | 48.464 | 1.00 | 17.66 |
| ATOM | 140 | N | PHE | 829 | 36.364 | 26.274 | 48.610 | 1.00 | 19.18 |
| ATOM | 141 | H | PHE | 829 | 35.972 | 25.448 | 48.288 | 1.00 | 0.00 |
| ATOM | 142 | CA | PHE | 829 | 35.615 | 27.111 | 49.551 | 1.00 | 20.77 |
| ATOM | 143 | CB | PHE | 829 | 34.283 | 27.549 | 48.830 | 1.00 | 18.59 |
| ATOM | 144 | CG | PHE | 829 | 33.580 | 28.581 | 49.584 | 1.00 | 17.13 |
| ATOM | 145 | CD1 | PHE | 829 | 34.111 | 29.864 | 49.573 | 1.00 | 16.92 |
| ATOM | 146 | CD2 | PHE | 829 | 32.383 | 28.284 | 50.276 | 1.00 | 15.90 |
| ATOM | 147 | CE1 | PHE | 829 | 33.438 | 30.897 | 50.256 | 1.00 | 16.79 |
| ATOM | 148 | CE2 | PHE | 829 | 31.729 | 29.305 | 50.949 | 1.00 | 17.61 |
| ATOM | 149 | CZ | PHE | 829 | 32.250 | 30.619 | 50.941 | 1.00 | 15.35 |
| ATOM | 150 | C | PHE | 829 | 35.328 | 26.377 | 50.887 | 1.00 | 21.99 |
| ATOM | 151 | O | PHE | 829 | 34.838 | 25.240 | 50.891 | 1.00 | 22.38 |
| ATOM | 152 | N | PRO | 830 | 35.578 | 27.014 | 52.044 | 1.00 | 23.77 |
| ATOM | 153 | CD | PRO | 830 | 35.858 | 28.459 | 52.242 | 1.00 | 23.83 |
| ATOM | 154 | CA | PRO | 830 | 35.365 | 26.345 | 53.331 | 1.00 | 24.88 |
| ATOM | 155 | CB | PRO | 830 | 35.780 | 27.352 | 54.416 | 1.00 | 23.20 |
| ATOM | 156 | CG | PRO | 830 | 36.515 | 28.424 | 53.626 | 1.00 | 23.46 |
| ATOM | 157 | C | PRO | 830 | 33.894 | 25.949 | 53.426 | 1.00 | 24.94 |
| ATOM | 158 | O | PRO | 830 | 32.973 | 26.719 | 53.200 | 1.00 | 23.66 |
| ATOM | 159 | N | ARG | 831 | 33.679 | 24.682 | 53.715 | 1.00 | 26.51 |
| ATOM | 160 | H | ARG | 831 | 34.424 | 24.071 | 53.545 | 1.00 | 0.00 |
| ATOM | 161 | CA | ARG | 831 | 32.299 | 24.146 | 53.816 | 1.00 | 27.84 |
| ATOM | 162 | CB | ARG | 831 | 32.552 | 22.539 | 53.866 | 1.00 | 28.49 |
| ATOM | 163 | CG | ARG | 831 | 31.535 | 21.408 | 53.361 | 1.00 | 32.00 |
| ATOM | 164 | CD | ARG | 831 | 31.843 | 20.222 | 52.306 | 1.00 | 28.95 |
| ATOM | 165 | NE | ARG | 831 | 33.099 | 19.650 | 52.672 | 1.00 | 28.62 |
| ATOM | 166 | HE | ARG | 831 | 33.355 | 19.801 | 53.606 | 1.00 | 0.00 |
| ATOM | 167 | CZ | ARG | 831 | 33.964 | 18.935 | 51.986 | 1.00 | 24.66 |
| ATOM | 168 | NH1 | ARG | 831 | 34.930 | 18.708 | 52.811 | 1.00 | 25.83 |
| ATOM | 169 | HH11 | ARG | 831 | 34.867 | 19.067 | 53.745 | 1.00 | 0.00 |
| ATOM | 170 | HH12 | ARG | 831 | 35.741 | 18.206 | 52.533 | 1.00 | 0.00 |
| ATOM | 171 | NH2 | ARG | 831 | 33.977 | 18.454 | 50.760 | 1.00 | 17.93 |
| ATOM | 172 | HH21 | ARG | 831 | 33.215 | 18.641 | 50.138 | 1.00 | 0.00 |
| ATOM | 173 | HH22 | ARG | 831 | 34.752 | 17.905 | 50.446 | 1.00 | 0.00 |
| ATOM | 174 | C | ARG | 831 | 31.601 | 24.832 | 54.993 | 1.00 | 26.14 |
| ATOM | 175 | O | ARG | 831 | 30.374 | 25.022 | 55.102 | 1.00 | 26.63 |
| ATOM | 176 | N | ASP | 832 | 32.422 | 25.310 | 55.897 | 1.00 | 25.97 |
| ATOM | 177 | H | ASP | 832 | 33.383 | 25.321 | 55.771 | 1.00 | 0.00 |
| ATOM | 178 | CA | ASP | 832 | 31.836 | 25.978 | 57.076 | 1.00 | 30.05 |
| ATOM | 179 | CB | ASP | 832 | 32.838 | 26.067 | 58.296 | 1.00 | 33.31 |
| ATOM | 180 | CG | ASP | 832 | 33.539 | 27.401 | 58.296 | 1.00 | 41.72 |
| ATOM | 181 | OD1 | ASP | 832 | 34.365 | 27.577 | 57.401 | 1.00 | 44.84 |
| ATOM | 182 | OD2 | ASP | 832 | 33.228 | 28.255 | 59.157 | 1.00 | 50.09 |
| ATOM | 183 | C | ASP | 832 | 31.355 | 27.380 | 56.735 | 1.00 | 28.36 |
| ATOM | 184 | O | ASP | 832 | 30.582 | 27.949 | 57.492 | 1.00 | 29.78 |
| ATOM | 185 | N | ARG | 833 | 31.871 | 27.986 | 55.670 | 1.00 | 25.87 |
| ATOM | 186 | H | ARG | 833 | 32.489 | 27.506 | 55.085 | 1.00 | 0.00 |
| ATOM | 187 | CA | ARG | 833 | 31.422 | 29.321 | 55.275 | 1.00 | 24.83 |
| ATOM | 188 | CB | ARG | 833 | 32.540 | 29.978 | 54.528 | 1.00 | 26.51 |
| ATOM | 189 | CG | ARG | 833 | 33.754 | 30.005 | 55.397 | 1.00 | 26.46 |
| ATOM | 190 | CD | ARG | 833 | 33.986 | 31.455 | 55.519 | 1.00 | 32.56 |
| ATOM | 191 | NE | ARG | 833 | 35.212 | 31.855 | 54.813 | 1.00 | 37.21 |
| ATOM | 192 | HE | ARG | 833 | 35.144 | 32.232 | 53.911 | 1.00 | 0.00 |
| ATOM | 193 | CZ | ARG | 833 | 36.449 | 31.714 | 55.351 | 1.00 | 39.61 |
| ATOM | 194 | NH1 | ARG | 833 | 37.507 | 32.149 | 54.636 | 1.00 | 41.87 |
| ATOM | 195 | HH11 | ARG | 833 | 37.363 | 32.552 | 53.734 | 1.00 | 0.00 |
| ATOM | 196 | HH12 | ARG | 833 | 38.429 | 32.059 | 55.012 | 1.00 | 0.00 |
| ATOM | 197 | NH2 | ARG | 833 | 36.686 | 31.116 | 56.539 | 1.00 | 40.59 |
| ATOM | 198 | HH21 | ARG | 833 | 35.937 | 30.743 | 57.090 | 1.00 | 0.00 |
| ATOM | 199 | HH22 | ARG | 833 | 37.621 | 31.039 | 56.884 | 1.00 | 0.00 |
| ATOM | 200 | C | ARG | 833 | 30.159 | 29.235 | 54.385 | 1.00 | 25.42 |
| ATOM | 201 | O | ARG | 833 | 29.630 | 30.241 | 53.872 | 1.00 | 24.74 |
| ATOM | 202 | N | LEU | 834 | 29.653 | 28.010 | 54.220 | 1.00 | 23.10 |
| ATOM | 203 | H | LEU | 834 | 29.980 | 27.279 | 54.778 | 1.00 | 0.00 |
| ATOM | 204 | CA | LEU | 834 | 28.503 | 27.748 | 53.375 | 1.00 | 24.99 |
| ATOM | 205 | CB | LEU | 834 | 28.924 | 26.645 | 52.324 | 1.00 | 24.70 |
| ATOM | 206 | CG | LEU | 834 | 27.933 | 26.307 | 51.208 | 1.00 | 23.02 |
| ATOM | 207 | CD1 | LEU | 834 | 27.749 | 27.560 | 50.358 | 1.00 | 22.95 |
| ATOM | 208 | CD2 | LEU | 834 | 28.441 | 25.151 | 50.352 | 1.00 | 22.08 |
| ATOM | 209 | C | LEU | 834 | 27.292 | 27.320 | 54.170 | 1.00 | 26.12 |
| ATOM | 210 | O | LEU | 834 | 27.356 | 26.345 | 54.899 | 1.00 | 30.06 |
| ATOM | 211 | N | LYS | 835 | 26.200 | 28.068 | 54.123 | 1.00 | 24.99 |
| ATOM | 212 | H | LYS | 835 | 26.280 | 28.952 | 53.711 | 1.00 | 0.00 |
| ATOM | 213 | CA | LYS | 835 | 24.969 | 27.699 | 54.820 | 1.00 | 23.53 |
| ATOM | 214 | CB | LYS | 835 | 24.285 | 28.946 | 55.482 | 1.00 | 26.78 |
| ATOM | 215 | C | LYS | 835 | 23.942 | 27.064 | 53.879 | 1.00 | 22.51 |
| ATOM | 216 | O | LYS | 835 | 23.306 | 27.727 | 53.047 | 1.00 | 21.37 |
| ATOM | 217 | N | LEU | 836 | 23.735 | 25.777 | 53.966 | 1.00 | 21.74 |
| ATOM | 218 | H | LEU | 836 | 24.232 | 25.272 | 54.647 | 1.00 | 0.00 |
| ATOM | 219 | CA | LEU | 836 | 22.744 | 25.116 | 53.094 | 1.00 | 24.15 |
| ATOM | 220 | CB | LEU | 836 | 23.011 | 23.599 | 53.124 | 1.00 | 26.57 |
| ATOM | 221 | CG | LEU | 836 | 23.945 | 22.988 | 52.011 | 1.00 | 27.55 |
| ATOM | 222 | CD1 | LEU | 836 | 25.142 | 23.796 | 51.575 | 1.00 | 23.41 |
| ATOM | 223 | CD2 | LEU | 836 | 24.456 | 21.735 | 52.633 | 1.00 | 25.98 |
| ATOM | 224 | C | LEU | 836 | 21.281 | 25.393 | 53.390 | 1.00 | 25.33 |
| ATOM | 225 | O | LEU | 836 | 20.877 | 25.502 | 54.529 | 1.00 | 27.67 |
| ATOM | 226 | N | GLY | 837 | 20.429 | 25.502 | 52.400 | 1.00 | 25.95 |
| ATOM | 227 | H | GLY | 837 | 20.786 | 25.405 | 51.495 | 1.00 | 0.00 |
| ATOM | 228 | CA | GLY | 837 | 19.025 | 25.763 | 52.649 | 1.00 | 26.93 |
| ATOM | 229 | C | GLY | 837 | 18.145 | 24.764 | 51.925 | 1.00 | 30.38 |
| ATOM | 230 | O | GLY | 837 | 18.432 | 23.567 | 51.831 | 1.00 | 31.75 |
| ATOM | 231 | N | LYS | 838 | 17.009 | 25.257 | 51.434 | 1.00 | 31.50 |
| ATOM | 232 | H | LYS | 838 | 16.818 | 26.198 | 51.616 | 1.00 | 0.00 |
| ATOM | 233 | CA | LYS | 838 | 16.001 | 24.501 | 50.686 | 1.00 | 32.46 |
| ATOM | 234 | CB | LYS | 838 | 14.841 | 25.452 | 50.513 | 1.00 | 36.07 |
| ATOM | 235 | CG | LYS | 838 | 13.587 | 24.613 | 50.442 | 1.00 | 44.63 |
| ATOM | 236 | CD | LYS | 838 | 12.738 | 24.785 | 49.141 | 1.00 | 50.25 |
| ATOM | 237 | CE | LYS | 838 | 11.497 | 23.808 | 49.086 | 1.00 | 52.36 |
| ATOM | 238 | NZ | LYS | 838 | 10.650 | 23.970 | 50.291 | 1.00 | 56.10 |
| ATOM | 239 | HZ1 | LYS | 838 | 10.491 | 24.982 | 50.474 | 1.00 | 0.00 |
| ATOM | 240 | HZ2 | LYS | 838 | 11.158 | 23.561 | 51.103 | 1.00 | 0.00 |
| ATOM | 241 | HZ3 | LYS | 838 | 9.739 | 23.483 | 50.174 | 1.00 | 0.00 |
| ATOM | 242 | C | LYS | 838 | 16.534 | 23.973 | 49.315 | 1.00 | 32.28 |
| ATOM | 243 | O | LYS | 838 | 17.271 | 24.643 | 48.567 | 1.00 | 29.60 |
| ATOM | 244 | N | PRO | 839 | 16.166 | 22.744 | 48.921 | 1.00 | 31.20 |
| ATOM | 245 | CD | PRO | 839 | 15.392 | 21.738 | 49.660 | 1.00 | 32.44 |
| ATOM | 246 | CA | PRO | 839 | 16.533 | 22.239 | 47.595 | 1.00 | 30.83 |
| ATOM | 247 | CB | PRO | 839 | 16.274 | 20.708 | 47.658 | 1.00 | 30.26 |
| ATOM | 248 | CG | PRO | 839 | 15.109 | 20.643 | 48.610 | 1.00 | 32.64 |
| ATOM | 249 | C | PRO | 839 | 15.786 | 22.930 | 46.456 | 1.00 | 27.62 |
| ATOM | 250 | O | PRO | 839 | 14.684 | 23.467 | 46.526 | 1.00 | 26.87 |
| ATOM | 251 | N | LEU | 840 | 16.578 | 23.096 | 45.439 | 1.00 | 26.69 |
| ATOM | 252 | H | LEU | 840 | 17.496 | 22.833 | 45.591 | 1.00 | 0.00 |
| ATOM | 253 | CA | LEU | 840 | 16.192 | 23.691 | 44.162 | 1.00 | 22.97 |
| ATOM | 254 | CB | LEU | 840 | 17.416 | 24.507 | 43.665 | 1.00 | 18.74 |
| ATOM | 255 | CG | LEU | 840 | 17.703 | 25.712 | 44.565 | 1.00 | 14.60 |
| ATOM | 256 | CD1 | LEU | 840 | 18.732 | 26.608 | 43.873 | 1.00 | 9.35 |
| ATOM | 257 | CD2 | LEU | 840 | 16.329 | 26.402 | 44.901 | 1.00 | 14.95 |
| ATOM | 258 | C | LEU | 840 | 15.744 | 22.560 | 43.210 | 1.00 | 24.05 |
| ATOM | 259 | O | LEU | 840 | 14.895 | 22.748 | 42.362 | 1.00 | 27.38 |
| ATOM | 260 | N | GLY | 841 | 16.318 | 21.378 | 43.249 | 1.00 | 21.91 |
| ATOM | 261 | H | GLY | 841 | 17.080 | 21.233 | 43.844 | 1.00 | 0.00 |
| ATOM | 262 | CA | GLY | 841 | 15.857 | 20.290 | 42.392 | 1.00 | 21.34 |
| ATOM | 263 | C | GLY | 841 | 16.505 | 19.000 | 42.865 | 1.00 | 25.48 |
| ATOM | 264 | O | GLY | 841 | 17.687 | 19.007 | 43.323 | 1.00 | 24.74 |
| ATOM | 265 | N | ARG | 842 | 15.736 | 17.922 | 42.986 | 1.00 | 25.17 |
| ATOM | 266 | H | ARG | 842 | 14.760 | 17.980 | 42.895 | 1.00 | 0.00 |
| ATOM | 267 | CA | ARG | 842 | 16.511 | 16.739 | 43.356 | 1.00 | 29.39 |
| ATOM | 268 | CB | ARG | 842 | 16.487 | 16.581 | 44.893 | 1.00 | 34.40 |
| ATOM | 269 | CG | ARG | 842 | 15.243 | 16.103 | 45.567 | 1.00 | 41.49 |
| ATOM | 270 | CD | ARG | 842 | 15.405 | 16.457 | 47.033 | 1.00 | 46.68 |
| ATOM | 271 | NE | ARG | 842 | 16.571 | 15.812 | 47.651 | 1.00 | 53.78 |
| ATOM | 272 | HE | ARG | 842 | 17.332 | 15.525 | 47.109 | 1.00 | 0.00 |
| ATOM | 273 | CZ | ARG | 842 | 16.654 | 15.687 | 48.997 | 1.00 | 59.00 |
| ATOM | 274 | NH1 | ARG | 842 | 15.665 | 16.157 | 49.818 | 1.00 | 60.15 |
| ATOM | 275 | HH11 | ARG | 842 | 14.858 | 16.599 | 49.429 | 1.00 | 0.00 |
| ATOM | 276 | HH12 | ARG | 842 | 15.752 | 16.051 | 50.808 | 1.00 | 0.00 |
| ATOM | 277 | NH2 | ARG | 842 | 17.743 | 15.067 | 49.529 | 1.00 | 61.37 |
| ATOM | 278 | HH21 | ARG | 842 | 18.468 | 14.720 | 48.934 | 1.00 | 0.00 |
| ATOM | 279 | HH22 | ARG | 842 | 17.823 | 14.963 | 50.520 | 1.00 | 0.00 |
| ATOM | 280 | C | ARG | 842 | 16.125 | 15.465 | 42.615 | 1.00 | 28.74 |
| ATOM | 281 | O | ARG | 842 | 15.010 | 15.266 | 42.113 | 1.00 | 27.27 |
| ATOM | 282 | N | GLY | 843 | 17.226 | 14.716 | 42.333 | 1.00 | 28.12 |
| ATOM | 283 | H | GLY | 843 | 18.061 | 14.960 | 42.788 | 1.00 | 0.00 |
| ATOM | 284 | CA | GLY | 843 | 17.186 | 13.470 | 41.578 | 1.00 | 25.47 |
| ATOM | 285 | C | GLY | 843 | 17.360 | 12.307 | 42.454 | 1.00 | 27.01 |
| ATOM | 286 | O | GLY | 843 | 17.223 | 12.388 | 43.660 | 1.00 | 26.48 |
| ATOM | 287 | N | ALA | 844 | 17.702 | 11.189 | 41.867 | 1.00 | 27.72 |
| ATOM | 288 | H | ALA | 844 | 17.926 | 11.235 | 40.914 | 1.00 | 0.00 |
| ATOM | 289 | CA | ALA | 844 | 17.866 | 9.949 | 42.657 | 1.00 | 28.07 |
| ATOM | 290 | CB | ALA | 844 | 17.908 | 8.698 | 41.674 | 1.00 | 28.68 |
| ATOM | 291 | C | ALA | 844 | 19.114 | 9.933 | 43.525 | 1.00 | 25.24 |
| ATOM | 292 | O | ALA | 844 | 19.129 | 9.465 | 44.654 | 1.00 | 24.49 |
| ATOM | 293 | N | PHE | 845 | 20.213 | 10.361 | 42.901 | 1.00 | 23.00 |
| ATOM | 294 | H | PHE | 845 | 19.977 | 10.695 | 42.034 | 1.00 | 0.00 |
| ATOM | 295 | CA | PHE | 845 | 21.549 | 10.395 | 43.502 | 1.00 | 21.41 |
| ATOM | 296 | CB | PHE | 845 | 22.553 | 9.751 | 42.608 | 1.00 | 23.99 |
| ATOM | 297 | CG | PHE | 845 | 22.144 | 8.405 | 42.259 | 1.00 | 28.00 |
| ATOM | 298 | CD1 | PHE | 845 | 22.225 | 8.052 | 40.904 | 1.00 | 31.51 |
| ATOM | 299 | CD2 | PHE | 845 | 21.674 | 7.496 | 43.225 | 1.00 | 32.46 |
| ATOM | 300 | CE1 | PHE | 845 | 21.831 | 6.774 | 40.496 | 1.00 | 33.90 |
| ATOM | 301 | CE2 | PHE | 845 | 21.270 | 6.211 | 42.828 | 1.00 | 35.93 |
| ATOM | 302 | CZ | PHE | 845 | 21.358 | 5.865 | 41.460 | 1.00 | 35.86 |
| ATOM | 303 | C | PHE | 845 | 22.050 | 11.771 | 43.765 | 1.00 | 22.16 |
| ATOM | 304 | O | PHE | 845 | 22.978 | 11.953 | 44.551 | 1.00 | 21.25 |
| ATOM | 305 | N | GLY | 846 | 21.536 | 12.769 | 43.078 | 1.00 | 20.31 |
| ATOM | 306 | H | GLY | 846 | 20.741 | 12.647 | 42.526 | 1.00 | 0.00 |
| ATOM | 307 | CA | GLY | 846 | 22.054 | 14.132 | 43.349 | 1.00 | 19.91 |
| ATOM | 308 | C | GLY | 846 | 21.023 | 15.171 | 43.680 | 1.00 | 19.36 |
| ATOM | 309 | O | GLY | 846 | 19.800 | 14.939 | 43.643 | 1.00 | 16.85 |
| ATOM | 310 | N | GLN | 847 | 21.515 | 16.365 | 43.966 | 1.00 | 17.97 |
| ATOM | 311 | H | GLN | 847 | 22.481 | 16.515 | 43.999 | 1.00 | 0.00 |
| ATOM | 312 | CA | GLN | 847 | 20.546 | 17.450 | 44.269 | 1.00 | 20.57 |
| ATOM | 313 | CB | GLN | 847 | 19.851 | 17.361 | 45.710 | 1.00 | 18.83 |
| ATOM | 314 | CG | GLN | 847 | 20.946 | 17.506 | 46.745 | 1.00 | 21.69 |
| ATOM | 315 | CD | GLN | 847 | 20.521 | 16.947 | 48.046 | 1.00 | 24.41 |
| ATOM | 316 | OE1 | GLN | 847 | 19.431 | 17.166 | 48.568 | 1.00 | 26.58 |
| ATOM | 317 | NE2 | GLN | 847 | 21.395 | 16.194 | 48.652 | 1.00 | 23.60 |
| ATOM | 318 | HE21 | GLN | 847 | 22.277 | 16.006 | 48.282 | 1.00 | 0.00 |
| ATOM | 319 | HE22 | GLN | 847 | 21.089 | 15.844 | 49.507 | 1.00 | 0.00 |
| ATOM | 320 | C | GLN | 847 | 21.245 | 18.794 | 44.210 | 1.00 | 20.00 |
| ATOM | 321 | O | GLN | 847 | 22.477 | 18.894 | 44.303 | 1.00 | 17.70 |
| ATOM | 322 | N | VAL | 848 | 20.412 | 19.785 | 43.885 | 1.00 | 19.47 |
| ATOM | 323 | H | VAL | 848 | 19.501 | 19.539 | 43.620 | 1.00 | 0.00 |
| ATOM | 324 | CA | VAL | 848 | 20.879 | 21.172 | 43.850 | 1.00 | 18.33 |
| ATOM | 325 | CB | VAL | 848 | 20.626 | 21.903 | 42.454 | 1.00 | 19.29 |
| ATOM | 326 | CG1 | VAL | 848 | 21.007 | 23.442 | 42.572 | 1.00 | 20.20 |
| ATOM | 327 | CG2 | VAL | 848 | 21.544 | 21.325 | 41.350 | 1.00 | 15.24 |
| ATOM | 328 | C | VAL | 848 | 20.082 | 21.869 | 44.968 | 1.00 | 17.07 |
| ATOM | 329 | O | VAL | 848 | 18.861 | 21.713 | 45.186 | 1.00 | 15.78 |
| ATOM | 330 | N | ILE | 849 | 20.837 | 22.490 | 45.850 | 1.00 | 18.39 |
| ATOM | 331 | H | ILE | 849 | 21.816 | 22.439 | 45.791 | 1.00 | 0.00 |
| ATOM | 332 | CA | ILE | 849 | 20.152 | 23.202 | 46.928 | 1.00 | 18.87 |
| ATOM | 333 | CB | ILE | 849 | 20.264 | 22.393 | 48.292 | 1.00 | 20.66 |
| ATOM | 334 | CG2 | ILE | 849 | 20.223 | 20.860 | 48.095 | 1.00 | 21.68 |
| ATOM | 335 | CG1 | ILE | 849 | 21.522 | 22.618 | 48.964 | 1.00 | 20.73 |
| ATOM | 336 | CD1 | ILE | 849 | 20.653 | 22.978 | 50.170 | 1.00 | 24.32 |
| ATOM | 337 | C | ILE | 849 | 20.635 | 24.598 | 47.093 | 1.00 | 18.34 |
| ATOM | 338 | O | ILE | 849 | 21.805 | 24.921 | 46.803 | 1.00 | 17.01 |
| ATOM | 339 | N | GLU | 850 | 19.680 | 25.472 | 47.379 | 1.00 | 19.23 |
| ATOM | 340 | H | GLU | 850 | 18.767 | 25.130 | 47.459 | 1.00 | 0.00 |
| ATOM | 341 | CA | GLU | 850 | 19.958 | 26.923 | 47.654 | 1.00 | 21.58 |
| ATOM | 342 | CB | GLU | 850 | 18.687 | 27.635 | 47.878 | 1.00 | 25.30 |
| ATOM | 343 | CG | GLU | 850 | 18.771 | 29.138 | 47.671 | 1.00 | 29.23 |
| ATOM | 344 | CD | GLU | 850 | 17.316 | 29.742 | 47.723 | 1.00 | 32.86 |
| ATOM | 345 | OE1 | GLU | 850 | 16.262 | 29.084 | 47.870 | 1.00 | 32.48 |
| ATOM | 346 | OE2 | GLU | 850 | 17.262 | 30.959 | 47.612 | 1.00 | 38.35 |
| ATOM | 347 | C | GLU | 850 | 20.796 | 27.084 | 48.940 | 1.00 | 21.55 |
| ATOM | 348 | O | GLU | 850 | 20.578 | 26.394 | 49.940 | 1.00 | 21.67 |
| ATOM | 349 | N | ALA | 851 | 21.737 | 28.026 | 48.944 | 1.00 | 20.99 |
| ATOM | 350 | H | ALA | 851 | 21.874 | 28.560 | 48.128 | 1.00 | 0.00 |
| ATOM | 351 | CA | ALA | 851 | 22.652 | 28.229 | 50.079 | 1.00 | 18.44 |
| ATOM | 352 | CB | ALA | 851 | 23.841 | 27.291 | 49.971 | 1.00 | 12.74 |
| ATOM | 353 | C | ALA | 851 | 23.208 | 29.593 | 50.138 | 1.00 | 16.98 |
| ATOM | 354 | O | ALA | 851 | 23.186 | 30.289 | 49.147 | 1.00 | 17.28 |
| ATOM | 355 | N | ASP | 852 | 23.718 | 29.998 | 51.274 | 1.00 | 16.74 |
| ATOM | 356 | H | ASP | 852 | 23.685 | 29.383 | 52.032 | 1.00 | 0.00 |
| ATOM | 357 | CA | ASP | 852 | 24.335 | 31.318 | 51.419 | 1.00 | 18.62 |
| ATOM | 358 | CB | ASP | 852 | 23.737 | 32.101 | 52.552 | 1.00 | 23.28 |
| ATOM | 359 | CG | ASP | 852 | 22.366 | 32.664 | 52.137 | 1.00 | 29.37 |
| ATOM | 360 | OD1 | ASP | 852 | 21.605 | 32.930 | 53.069 | 1.00 | 32.54 |
| ATOM | 361 | OD2 | ASP | 852 | 22.054 | 32.865 | 50.947 | 1.00 | 30.95 |
| ATOM | 362 | C | ASP | 852 | 25.771 | 31.203 | 51.688 | 1.00 | 16.79 |
| ATOM | 363 | O | ASP | 852 | 26.164 | 30.522 | 52.619 | 1.00 | 18.69 |
| ATOM | 364 | N | ALA | 853 | 26.568 | 31.799 | 50.846 | 1.00 | 18.01 |
| ATOM | 365 | H | ALA | 853 | 26.141 | 32.254 | 50.095 | 1.00 | 0.00 |
| ATOM | 366 | CA | ALA | 853 | 28.020 | 31.719 | 50.925 | 1.00 | 17.93 |
| ATOM | 367 | CB | ALA | 853 | 28.586 | 31.268 | 49.558 | 1.00 | 15.00 |
| ATOM | 368 | C | ALA | 853 | 28.583 | 33.073 | 51.296 | 1.00 | 18.87 |
| ATOM | 369 | O | ALA | 853 | 28.260 | 34.086 | 50.666 | 1.00 | 21.14 |
| ATOM | 370 | N | PHE | 854 | 29.347 | 33.097 | 52.366 | 1.00 | 20.21 |
| ATOM | 371 | H | PHE | 854 | 29.505 | 32.247 | 52.839 | 1.00 | 0.00 |
| ATOM | 372 | CA | PHE | 854 | 29.964 | 34.333 | 52.857 | 1.00 | 23.26 |
| ATOM | 373 | CB | PHE | 854 | 30.157 | 34.244 | 54.432 | 1.00 | 23.23 |
| ATOM | 374 | CG | PHE | 854 | 30.752 | 35.487 | 55.069 | 1.00 | 22.08 |
| ATOM | 375 | CD1 | PHE | 854 | 32.145 | 35.632 | 55.302 | 1.00 | 22.82 |
| ATOM | 376 | CD2 | PHE | 854 | 29.882 | 36.516 | 55.467 | 1.00 | 21.58 |
| ATOM | 377 | CE1 | PHE | 854 | 32.666 | 36.781 | 55.929 | 1.00 | 21.72 |
| ATOM | 378 | CE2 | PHE | 854 | 30.399 | 37.656 | 56.093 | 1.00 | 21.70 |
| ATOM | 379 | CZ | PHE | 854 | 31.769 | 37.790 | 56.317 | 1.00 | 19.75 |
| ATOM | 380 | C | PHE | 854 | 31.307 | 34.624 | 52.156 | 1.00 | 21.77 |
| ATOM | 381 | O | PHE | 854 | 32.301 | 33.942 | 52.381 | 1.00 | 25.59 |
| ATOM | 382 | N | GLY | 855 | 31.341 | 35.619 | 51.294 | 1.00 | 19.83 |
| ATOM | 383 | H | GLY | 855 | 30.488 | 36.051 | 51.160 | 1.00 | 0.00 |
| ATOM | 384 | CA | GLY | 855 | 32.547 | 36.005 | 50.616 | 1.00 | 19.31 |
| ATOM | 385 | C | GLY | 855 | 32.936 | 35.027 | 49.576 | 1.00 | 20.01 |
| ATOM | 386 | O | GLY | 855 | 34.124 | 34.846 | 49.379 | 1.00 | 23.08 |
| ATOM | 387 | N | ILE | 856 | 32.031 | 34.347 | 48.894 | 1.00 | 19.76 |
| ATOM | 388 | H | ILE | 856 | 31.085 | 34.403 | 49.111 | 1.00 | 0.00 |
| ATOM | 389 | CA | ILE | 856 | 32.583 | 33.413 | 47.879 | 1.00 | 20.71 |
| ATOM | 390 | CB | ILE | 856 | 31.454 | 32.339 | 47.436 | 1.00 | 18.43 |
| ATOM | 391 | CG2 | ILE | 856 | 30.343 | 32.921 | 46.571 | 1.00 | 17.16 |
| ATOM | 392 | CG1 | ILE | 856 | 32.202 | 31.214 | 46.705 | 1.00 | 17.68 |
| ATOM | 393 | CD1 | ILE | 856 | 31.518 | 29.879 | 46.877 | 1.00 | 18.55 |
| ATOM | 394 | C | ILE | 856 | 33.144 | 34.215 | 46.680 | 1.00 | 23.23 |
| ATOM | 395 | O | ILE | 856 | 34.127 | 33.845 | 46.038 | 1.00 | 23.54 |
| ATOM | 396 | N | ASP | 857 | 32.542 | 35.332 | 46.329 | 1.00 | 24.71 |
| ATOM | 397 | H | ASP | 857 | 31.753 | 35.596 | 46.837 | 1.00 | 0.00 |
| ATOM | 398 | CA | ASP | 857 | 33.108 | 36.070 | 45.218 | 1.00 | 30.35 |
| ATOM | 399 | CB | ASP | 857 | 32.132 | 36.039 | 44.039 | 1.00 | 33.66 |
| ATOM | 1 | CG | ASP | 857 | 30.709 | 36.547 | 44.390 | 1.00 | 40.65 |
| ATOM | 401 | OD1 | ASP | 857 | 30.485 | 37.237 | 45.398 | 1.00 | 40.52 |
| ATOM | 402 | OD2 | ASP | 857 | 29.806 | 36.245 | 43.595 | 1.00 | 45.26 |
| ATOM | 403 | C | ASP | 857 | 33.429 | 37.505 | 45.667 | 1.00 | 34.14 |
| ATOM | 404 | O | ASP | 857 | 34.470 | 38.003 | 45.201 | 1.00 | 37.20 |
| ATOM | 405 | N | LYS | 858 | 32.640 | 38.231 | 46.529 | 1.00 | 32.74 |
| ATOM | 406 | H | LYS | 858 | 31.927 | 37.770 | 47.016 | 1.00 | 0.00 |
| ATOM | 407 | CA | LYS | 858 | 33.156 | 39.601 | 46.916 | 1.00 | 33.37 |
| ATOM | 408 | CB | LYS | 858 | 32.174 | 40.846 | 46.788 | 1.00 | 32.21 |
| ATOM | 409 | C | LYS | 858 | 33.532 | 39.475 | 48.361 | 1.00 | 31.38 |
| ATOM | 410 | O | LYS | 858 | 32.805 | 38.902 | 49.188 | 1.00 | 31.73 |
| ATOM | 411 | N | THR | 859 | 34.684 | 40.005 | 48.686 | 1.00 | 27.89 |
| ATOM | 412 | H | THR | 859 | 35.160 | 40.586 | 48.066 | 1.00 | 0.00 |
| ATOM | 413 | CA | THR | 859 | 35.114 | 39.768 | 50.058 | 1.00 | 28.65 |
| ATOM | 414 | CB | THR | 859 | 36.504 | 40.447 | 50.339 | 1.00 | 30.47 |
| ATOM | 415 | OG1 | TER | 859 | 37.407 | 40.084 | 49.302 | 1.00 | 32.16 |
| ATOM | 416 | HG1 | THR | 859 | 38.260 | 40.492 | 49.474 | 1.00 | 0.00 |
| ATOM | 417 | CG2 | THR | 859 | 37.120 | 40.016 | 51.722 | 1.00 | 28.53 |
| ATOM | 418 | C | THR | 859 | 34.126 | 40.188 | 51.139 | 1.00 | 25.75 |
| ATOM | 419 | O | THR | 859 | 33.443 | 41.199 | 51.042 | 1.00 | 25.26 |
| ATOM | 420 | N | ALA | 860 | 34.034 | 39.309 | 52.141 | 1.00 | 24.60 |
| ATOM | 421 | H | ALA | 860 | 34.440 | 38.446 | 51.913 | 1.00 | 0.00 |
| ATOM | 422 | CA | ALA | 860 | 33.179 | 39.427 | 53.322 | 1.00 | 21.80 |
| ATOM | 423 | CB | ALA | 860 | 33.810 | 40.491 | 54.254 | 1.00 | 19.69 |
| ATOM | 424 | C | ALA | 860 | 31.708 | 39.759 | 52.973 | 1.00 | 21.64 |
| ATOM | 425 | O | ALA | 860 | 30.981 | 40.387 | 53.717 | 1.00 | 21.05 |
| ATOM | 426 | N | THR | 861 | 31.174 | 39.374 | 51.816 | 1.00 | 22.92 |
| ATOM | 427 | H | THR | 861 | 31.753 | 39.004 | 51.117 | 1.00 | 0.00 |
| ATOM | 428 | CA | THR | 861 | 29.725 | 39.645 | 51.543 | 1.00 | 23.38 |
| ATOM | 429 | CB | THR | 861 | 29.475 | 40.690 | 50.363 | 1.00 | 25.50 |
| ATOM | 430 | OG1 | THR | 861 | 28.458 | 40.282 | 49.431 | 1.00 | 28.59 |
| ATOM | 431 | HG1 | THR | 861 | 28.170 | 41.038 | 48.910 | 1.00 | 0.00 |
| ATOM | 432 | CG2 | THR | 861 | 30.771 | 40.931 | 49.657 | 1.00 | 24.79 |
| ATOM | 433 | C | THR | 861 | 29.008 | 38.360 | 51.259 | 1.00 | 21.82 |
| ATOM | 434 | O | THR | 861 | 29.540 | 37.465 | 50.606 | 1.00 | 21.00 |
| ATOM | 435 | N | CYS | 862 | 27.946 | 38.148 | 52.010 | 1.00 | 21.09 |
| ATOM | 436 | H | CYS | 862 | 27.715 | 38.898 | 52.604 | 1.00 | 0.00 |
| ATOM | 437 | CA | CYS | 862 | 27.131 | 36.922 | 51.848 | 1.00 | 25.52 |
| ATOM | 438 | CB | CYS | 862 | 25.840 | 36.775 | 52.758 | 1.00 | 24.20 |
| ATOM | 439 | SG | CYS | 862 | 25.596 | 35.108 | 53.423 | 1.00 | 33.71 |
| ATOM | 440 | C | CYS | 862 | 26.593 | 36.981 | 50.424 | 1.00 | 26.68 |
| ATOM | 441 | O | CYS | 862 | 26.207 | 38.068 | 49.954 | 1.00 | 26.42 |
| ATOM | 442 | N | ARG | 863 | 26.462 | 35.814 | 49.779 | 1.00 | 25.04 |
| ATOM | 443 | H | ARG | 863 | 26.680 | 34.993 | 50.257 | 1.00 | 0.00 |
| ATOM | 444 | CA | ARG | 863 | 25.989 | 35.714 | 48.383 | 1.00 | 22.97 |
| ATOM | 445 | CB | ARG | 863 | 27.325 | 35.814 | 47.618 | 1.00 | 27.95 |
| ATOM | 446 | CG | ARG | 863 | 27.670 | 35.112 | 46.303 | 1.00 | 33.24 |
| ATOM | 447 | CD | ARG | 863 | 26.567 | 35.291 | 45.289 | 1.00 | 33.37 |
| ATOM | 448 | NE | ARG | 863 | 27.198 | 35.372 | 43.947 | 1.00 | 37.98 |
| ATOM | 449 | HE | ARG | 863 | 28.014 | 35.871 | 43.906 | 1.00 | 0.00 |
| ATOM | 450 | CZ | ARG | 863 | 26.636 | 34.923 | 42.798 | 1.00 | 39.52 |
| ATOM | 451 | NH1 | ARG | 863 | 27.399 | 35.132 | 41.719 | 1.00 | 37.87 |
| ATOM | 452 | HH11 | ARG | 863 | 28.290 | 35.580 | 41.816 | 1.00 | 0.00 |
| ATOM | 453 | HH12 | ARG | 863 | 27.068 | 34.851 | 40.821 | 1.00 | 0.00 |
| ATOM | 454 | NH2 | ARG | 863 | 25.426 | 34.252 | 42.733 | 1.00 | 35.98 |
| ATOM | 455 | HH21 | ARG | 863 | 24.911 | 34.065 | 43.562 | 1.00 | 0.00 |
| ATOM | 456 | HH22 | ARG | 863 | 25.066 | 33.951 | 41.856 | 1.00 | 0.00 |
| ATOM | 457 | C | ARG | 863 | 25.196 | 34.445 | 48.257 | 1.00 | 21.11 |
| ATOM | 458 | O | ARG | 863 | 25.636 | 33.407 | 48.740 | 1.00 | 19.89 |
| ATOM | 459 | N | THR | 864 | 23.929 | 34.538 | 47.877 | 1.00 | 19.22 |
| ATOM | 460 | H | THR | 864 | 23.577 | 35.440 | 47.791 | 1.00 | 0.00 |
| ATOM | 461 | CA | THR | 864 | 23.104 | 33.319 | 47.749 | 1.00 | 18.46 |
| ATOM | 462 | CB | THR | 864 | 21.624 | 33.559 | 47.417 | 1.00 | 18.89 |
| ATOM | 463 | OG1 | THR | 864 | 21.248 | 34.467 | 48.415 | 1.00 | 22.68 |
| ATOM | 464 | HG1 | THR | 864 | 20.286 | 34.565 | 48.392 | 1.00 | 0.00 |
| ATOM | 465 | CG2 | THR | 864 | 20.641 | 32.375 | 47.503 | 1.00 | 13.50 |
| ATOM | 466 | C | THR | 864 | 23.636 | 32.592 | 46.616 | 1.00 | 16.84 |
| ATOM | 467 | O | THR | 864 | 23.822 | 33.203 | 45.583 | 1.00 | 18.24 |
| ATOM | 468 | N | VAL | 865 | 23.881 | 31.335 | 46.752 | 1.00 | 16.27 |
| ATOM | 469 | H | VAL | 865 | 23.764 | 30.931 | 47.614 | 1.00 | 0.00 |
| ATOM | 470 | CA | VAL | 865 | 24.412 | 30.496 | 45.656 | 1.00 | 14.82 |
| ATOM | 471 | CB | VAL | 865 | 25.877 | 30.113 | 45.888 | 1.00 | 12.89 |
| ATOM | 472 | CG1 | VAL | 865 | 26.704 | 31.365 | 45.771 | 1.00 | 10.92 |
| ATOM | 473 | CG2 | VAL | 865 | 25.998 | 29.257 | 47.164 | 1.00 | 13.94 |
| ATOM | 474 | C | VAL | 865 | 23.626 | 29.179 | 45.527 | 1.00 | 14.86 |
| ATOM | 475 | O | VAL | 865 | 22.733 | 28.940 | 46.371 | 1.00 | 14.29 |
| ATOM | 476 | N | ALA | 866 | 23.822 | 28.435 | 44.404 | 1.00 | 12.75 |
| ATOM | 477 | H | ALA | 866 | 24.470 | 28.763 | 43.735 | 1.00 | 0.00 |
| ATOM | 478 | CA | ALA | 866 | 23.179 | 27.100 | 44.203 | 1.00 | 12.65 |
| ATOM | 479 | CB | ALA | 866 | 22.515 | 26.865 | 42.839 | 1.00 | 12.24 |
| ATOM | 480 | C | ALA | 866 | 24.318 | 26.105 | 44.244 | 1.00 | 12.26 |
| ATOM | 481 | O | ALA | 866 | 25.412 | 26.342 | 43.728 | 1.00 | 12.83 |
| ATOM | 482 | N | VAL | 867 | 24.141 | 25.032 | 45.016 | 1.00 | 16.67 |
| ATOM | 483 | H | VAL | 867 | 23.338 | 25.016 | 45.573 | 1.00 | 0.00 |
| ATOM | 484 | CA | VAL | 867 | 25.162 | 23.946 | 45.164 | 1.00 | 16.29 |
| ATOM | 485 | CB | VAL | 867 | 25.922 | 23.996 | 46.629 | 1.00 | 17.94 |
| ATOM | 486 | CG1 | VAL | 867 | 25.377 | 25.177 | 47.440 | 1.00 | 16.71 |
| ATOM | 487 | CG2 | VAL | 867 | 25.966 | 22.627 | 47.306 | 1.00 | 12.67 |
| ATOM | 488 | C | VAL | 867 | 24.671 | 22.531 | 44.866 | 1.00 | 15.37 |
| ATOM | 489 | O | VAL | 867 | 23.623 | 22.048 | 45.295 | 1.00 | 13.02 |
| ATOM | 490 | N | LYS | 868 | 25.393 | 21.963 | 43.884 | 1.00 | 14.85 |
| ATOM | 491 | H | LYS | 868 | 26.099 | 22.495 | 43.472 | 1.00 | 0.00 |
| ATOM | 492 | CA | LYS | 868 | 25.109 | 20.599 | 43.458 | 1.00 | 13.90 |
| ATOM | 493 | CB | LYS | 868 | 25.436 | 20.417 | 41.941 | 1.00 | 13.63 |
| ATOM | 494 | CG | LYS | 868 | 24.976 | 19.030 | 41.484 | 1.00 | 12.67 |
| ATOM | 495 | CD | LYS | 868 | 25.184 | 18.782 | 39.992 | 1.00 | 16.68 |
| ATOM | 496 | CE | LYS | 868 | 24.168 | 19.457 | 39.059 | 1.00 | 15.81 |
| ATOM | 497 | NZ | LYS | 868 | 24.490 | 19.225 | 37.661 | 1.00 | 13.96 |
| ATOM | 498 | HZ1 | LYS | 868 | 24.862 | 18.263 | 37.533 | 1.00 | 0.00 |
| ATOM | 499 | HZ2 | LYS | 868 | 25.231 | 19.903 | 37.387 | 1.00 | 0.00 |
| ATOM | 500 | HZ3 | LYS | 868 | 23.639 | 19.364 | 37.080 | 1.00 | 0.00 |
| ATOM | 501 | C | LYS | 868 | 25.960 | 19.633 | 44.335 | 1.00 | 13.89 |
| ATOM | 502 | O | LYS | 868 | 27.167 | 19.743 | 44.574 | 1.00 | 13.37 |
| ATOM | 503 | N | MET | 869 | 25.298 | 18.624 | 44.863 | 1.00 | 14.18 |
| ATOM | 504 | H | MET | 869 | 24.333 | 18.572 | 44.704 | 1.00 | 0.00 |
| ATOM | 505 | CA | MET | 869 | 25.967 | 17.638 | 45.683 | 1.00 | 12.99 |
| ATOM | 506 | CB | MET | 869 | 26.042 | 18.150 | 47.128 | 1.00 | 15.09 |
| ATOM | 507 | CG | MET | 869 | 24.635 | 18.450 | 47.700 | 1.00 | 21.92 |
| ATOM | 508 | SD | MET | 869 | 24.680 | 19.484 | 49.204 | 1.00 | 28.23 |
| ATOM | 509 | CE | MET | 869 | 25.553 | 18.169 | 50.135 | 1.00 | 30.94 |
| ATOM | 510 | C | MET | 869 | 25.188 | 16.362 | 45.606 | 1.00 | 12.87 |
| ATOM | 511 | O | MET | 869 | 24.090 | 16.305 | 45.058 | 1.00 | 9.95 |
| ATOM | 512 | N | LEU | 870 | 25.846 | 15.295 | 46.052 | 1.00 | 14.34 |
| ATOM | 513 | H | LEU | 870 | 26.802 | 15.455 | 46.196 | 1.00 | 0.00 |
| ATOM | 514 | CA | LEU | 870 | 25.210 | 13.972 | 46.054 | 1.00 | 15.43 |
| ATOM | 515 | CB | LEU | 870 | 26.306 | 12.826 | 46.023 | 1.00 | 12.70 |
| ATOM | 516 | CG | LEU | 870 | 27.237 | 12.881 | 44.725 | 1.00 | 12.04 |
| ATOM | 517 | CD1 | LEU | 870 | 28.140 | 11.685 | 44.629 | 1.00 | 11.74 |
| ATOM | 518 | CD2 | LEU | 870 | 26.355 | 12.896 | 43.462 | 1.00 | 14.31 |
| ATOM | 519 | C | LEU | 870 | 24.233 | 13.743 | 47.195 | 1.00 | 17.48 |
| ATOM | 520 | O | LEU | 870 | 24.181 | 14.445 | 48.206 | 1.00 | 18.40 |
| ATOM | 521 | N | LYS | 871 | 23.439 | 12.738 | 47.008 | 1.00 | 19.50 |
| ATOM | 522 | H | LYS | 871 | 23.622 | 12.169 | 46.240 | 1.00 | 0.00 |
| ATOM | 523 | CA | LYS | 871 | 22.405 | 12.293 | 47.935 | 1.00 | 25.00 |
| ATOM | 524 | CB | LYS | 871 | 21.027 | 12.294 | 47.208 | 1.00 | 25.95 |
| ATOM | 525 | CG | LYS | 871 | 20.377 | 13.652 | 47.241 | 1.00 | 30.09 |
| ATOM | 526 | CD | LYS | 871 | 19.213 | 13.683 | 46.252 | 1.00 | 34.75 |
| ATOM | 527 | CE | LYS | 871 | 17.907 | 13.068 | 46.806 | 1.00 | 37.77 |
| ATOM | 528 | NZ | LYS | 871 | 17.956 | 11.612 | 46.687 | 1.00 | 40.86 |
| ATOM | 529 | HZ1 | LYS | 871 | 18.875 | 11.275 | 47.041 | 1.00 | 0.00 |
| ATOM | 530 | HZ2 | LYS | 871 | 17.875 | 11.357 | 45.680 | 1.00 | 0.00 |
| ATOM | 531 | HZ3 | LYS | 871 | 17.192 | 11.171 | 47.236 | 1.00 | 0.00 |
| ATOM | 532 | C | LYS | 871 | 22.656 | 10.887 | 48.441 | 1.00 | 23.28 |
| ATOM | 533 | O | LYS | 871 | 23.443 | 10.143 | 47.870 | 1.00 | 23.98 |
| ATOM | 534 | N | GLU | 872 | 21.956 | 10.481 | 49.493 | 1.00 | 26.99 |
| ATOM | 535 | H | GLU | 872 | 21.274 | 11.091 | 49.854 | 1.00 | 0.00 |
| ATOM | 536 | CA | GLU | 872 | 22.021 | 9.095 | 50.057 | 1.00 | 28.92 |
| ATOM | 537 | CB | GLU | 872 | 20.988 | 8.945 | 51.229 | 1.00 | 38.58 |
| ATOM | 538 | CG | GLU | 872 | 19.993 | 7.713 | 51.323 | 1.00 | 51.19 |
| ATOM | 539 | CD | GLU | 872 | 20.612 | 6.378 | 51.895 | 1.00 | 60.10 |
| ATOM | 540 | OE1 | GLU | 872 | 19.872 | 5.361 | 51.821 | 1.00 | 61.87 |
| ATOM | 541 | OE2 | GLU | 872 | 21.785 | 6.366 | 52.399 | 1.00 | 62.32 |
| ATOM | 542 | C | GLU | 872 | 21.640 | 8.170 | 48.915 | 1.00 | 25.21 |
| ATOM | 543 | O | GLU | 872 | 20.628 | 8.378 | 48.235 | 1.00 | 24.50 |
| ATOM | 544 | N | GLY | 873 | 22.464 | 7.179 | 48.644 | 1.00 | 23.05 |
| ATOM | 545 | H | GLY | 873 | 23.247 | 6.967 | 49.198 | 1.00 | 0.00 |
| ATOM | 546 | CA | GLY | 873 | 22.113 | 6.317 | 47.529 | 1.00 | 21.78 |
| ATOM | 547 | C | GLY | 873 | 23.072 | 6.433 | 46.392 | 1.00 | 22.04 |
| ATOM | 548 | O | GLY | 873 | 23.187 | 5.544 | 45.556 | 1.00 | 19.52 |
| ATOM | 549 | N | ALA | 874 | 23.729 | 7.589 | 46.298 | 1.00 | 21.91 |
| ATOM | 550 | H | ALA | 874 | 23.505 | 8.338 | 46.890 | 1.00 | 0.00 |
| ATOM | 551 | CA | ALA | 874 | 24.690 | 7.725 | 45.204 | 1.00 | 20.44 |
| ATOM | 552 | CB | ALA | 874 | 25.234 | 9.133 | 45.041 | 1.00 | 21.31 |
| ATOM | 553 | C | ALA | 874 | 25.857 | 6.884 | 45.592 | 1.00 | 18.54 |
| ATOM | 554 | O | ALA | 874 | 26.098 | 6.600 | 46.763 | 1.00 | 17.52 |
| ATOM | 555 | N | THR | 875 | 26.696 | 6.604 | 44.635 | 1.00 | 19.07 |
| ATOM | 556 | H | THR | 875 | 26.521 | 7.009 | 43.769 | 1.00 | 0.00 |
| ATOM | 557 | CA | THR | 875 | 27.884 | 5.778 | 44.878 | 1.00 | 17.43 |
| ATOM | 558 | CB | THR | 875 | 27.842 | 4.440 | 44.037 | 1.00 | 19.39 |
| ATOM | 559 | OG1 | THR | 875 | 27.927 | 4.797 | 42.666 | 1.00 | 16.99 |
| ATOM | 560 | HG1 | THR | 875 | 27.856 | 3.965 | 42.165 | 1.00 | 0.00 |
| ATOM | 561 | CG2 | THR | 875 | 26.558 | 3.609 | 44.137 | 1.00 | 19.24 |
| ATOM | 562 | C | THR | 875 | 29.135 | 6.589 | 44.429 | 1.00 | 18.29 |
| ATOM | 563 | O | THR | 875 | 29.053 | 7.664 | 43.876 | 1.00 | 17.02 |
| ATOM | 564 | N | HIS | 876 | 30.373 | 6.096 | 44.622 | 1.00 | 20.06 |
| ATOM | 565 | H | HIS | 876 | 30.528 | 5.427 | 45.333 | 1.00 | 0.00 |
| ATOM | 566 | CA | HIS | 876 | 31.548 | 6.794 | 44.177 | 1.00 | 19.20 |
| ATOM | 567 | C | HIS | 876 | 31.533 | 7.069 | 42.681 | 1.00 | 18.07 |
| ATOM | 568 | O | HIS | 876 | 32.203 | 8.003 | 42.234 | 1.00 | 23.03 |
| ATOM | 569 | CB | HIS | 876 | 32.750 | 5.963 | 44.499 | 1.00 | 14.17 |
| ATOM | 570 | CG | HIS | 876 | 33.890 | 6.762 | 44.164 | 1.00 | 14.57 |
| ATOM | 571 | ND1 | HIS | 876 | 34.290 | 7.844 | 44.866 | 1.00 | 14.41 |
| ATOM | 572 | HD1 | HIS | 876 | 33.888 | 8.205 | 45.686 | 1.00 | 0.00 |
| ATOM | 573 | CD2 | HIS | 876 | 34.872 | 6.446 | 43.226 | 1.00 | 18.31 |
| ATOM | 574 | NE2 | HIS | 876 | 35.878 | 7.361 | 43.401 | 1.00 | 19.64 |
| ATOM | 575 | CE1 | HIS | 876 | 35.521 | 8.225 | 44.406 | 1.00 | 13.24 |
| ATOM | 576 | N | SER | 877 | 30.830 | 6.319 | 41.851 | 1.00 | 15.60 |
| ATOM | 577 | H | SER | 877 | 30.278 | 5.622 | 42.245 | 1.00 | 0.00 |
| ATOM | 578 | CA | SER | 877 | 30.807 | 6.618 | 40.435 | 1.00 | 14.53 |
| ATOM | 579 | CB | SER | 877 | 30.242 | 5.398 | 39.651 | 1.00 | 19.06 |
| ATOM | 580 | OG | SER | 877 | 28.905 | 5.029 | 39.882 | 1.00 | 30.21 |
| ATOM | 581 | HG | SER | 877 | 28.556 | 4.817 | 39.014 | 1.00 | 0.00 |
| ATOM | 582 | C | SER | 877 | 30.078 | 7.910 | 40.131 | 1.00 | 12.84 |
| ATOM | 583 | O | SER | 877 | 30.475 | 8.664 | 39.255 | 1.00 | 12.98 |
| ATOM | 584 | N | GLU | 878 | 29.063 | 8.256 | 40.916 | 1.00 | 13.37 |
| ATOM | 585 | H | GLU | 878 | 28.758 | 7.578 | 41.555 | 1.00 | 0.00 |
| ATOM | 586 | CA | GLU | 878 | 28.340 | 9.536 | 40.832 | 1.00 | 14.05 |
| ATOM | 587 | CB | GLU | 878 | 27.146 | 9.513 | 41.684 | 1.00 | 15.63 |
| ATOM | 588 | CG | GLU | 878 | 25.968 | 8.911 | 40.918 | 1.00 | 19.49 |
| ATOM | 589 | CD | GLU | 878 | 25.981 | 7.382 | 40.893 | 1.00 | 23.08 |
| ATOM | 590 | OE1 | GLU | 878 | 26.364 | 6.726 | 41.834 | 1.00 | 25.19 |
| ATOM | 591 | OE2 | GLU | 878 | 25.518 | 6.808 | 39.940 | 1.00 | 29.90 |
| ATOM | 592 | C | GLU | 878 | 29.278 | 10.612 | 41.344 | 1.00 | 14.66 |
| ATOM | 593 | O | GLU | 878 | 29.333 | 11.733 | 40.855 | 1.00 | 17.36 |
| ATOM | 594 | N | HIS | 879 | 30.091 | 10.280 | 42.296 | 1.00 | 14.55 |
| ATOM | 595 | H | HIS | 879 | 29.973 | 9.380 | 42.670 | 1.00 | 0.00 |
| ATOM | 596 | CA | HIS | 879 | 31.115 | 11.165 | 42.843 | 1.00 | 13.21 |
| ATOM | 597 | C | HIS | 879 | 32.147 | 11.490 | 41.781 | 1.00 | 15.27 |
| ATOM | 598 | O | HIS | 879 | 32.606 | 12.637 | 41.630 | 1.00 | 12.98 |
| ATOM | 599 | CB | HIS | 879 | 31.746 | 10.449 | 44.042 | 1.00 | 10.76 |
| ATOM | 600 | CG | HIS | 879 | 32.610 | 11.366 | 44.726 | 1.00 | 7.90 |
| ATOM | 601 | ND1 | HIS | 879 | 33.898 | 11.530 | 44.410 | 1.00 | 12.19 |
| ATOM | 602 | HD1 | HIS | 879 | 34.367 | 11.123 | 43.650 | 1.00 | 0.00 |
| ATOM | 603 | CD2 | HIS | 879 | 32.296 | 12.166 | 45.813 | 1.00 | 9.79 |
| ATOM | 604 | NE2 | HIS | 879 | 33.447 | 12.800 | 46.113 | 1.00 | 10.19 |
| ATOM | 605 | CE1 | HIS | 879 | 34.451 | 12.412 | 45.260 | 1.00 | 7.48 |
| ATOM | 606 | N | ARG | 880 | 32.587 | 10.482 | 41.027 | 1.00 | 17.24 |
| ATOM | 607 | H | ARG | 880 | 32.304 | 9.576 | 41.231 | 1.00 | 0.00 |
| ATOM | 608 | CA | ARG | 880 | 33.552 | 10.730 | 39.938 | 1.00 | 16.14 |
| ATOM | 609 | CB | ARG | 880 | 33.991 | 9.412 | 39.255 | 1.00 | 17.35 |
| ATOM | 610 | CG | ARG | 880 | 34.894 | 8.625 | 40.159 | 1.00 | 18.14 |
| ATOM | 611 | CD | ARG | 880 | 35.507 | 7.363 | 39.474 | 1.00 | 22.20 |
| ATOM | 612 | NE | ARG | 880 | 34.675 | 6.156 | 39.535 | 1.00 | 28.41 |
| ATOM | 613 | HE | ARG | 880 | 33.769 | 6.190 | 39.161 | 1.00 | 0.00 |
| ATOM | 614 | CZ | ARG | 880 | 35.051 | 4.967 | 40.064 | 1.00 | 28.73 |
| ATOM | 615 | NH1 | ARG | 880 | 34.169 | 3.994 | 39.990 | 1.00 | 32.49 |
| ATOM | 616 | HH11 | ARG | 880 | 33.274 | 4.126 | 39.565 | 1.00 | 0.00 |
| ATOM | 617 | HH12 | ARG | 880 | 34.420 | 3.104 | 40.371 | 1.00 | 0.00 |
| ATOM | 618 | NH2 | ARG | 880 | 36.180 | 4.685 | 40.721 | 1.00 | 28.04 |
| ATOM | 619 | HH21 | ARG | 880 | 36.857 | 5.406 | 40.861 | 1.00 | 0.00 |
| ATOM | 620 | HH22 | ARG | 880 | 36.354 | 3.767 | 41.075 | 1.00 | 0.00 |
| ATOM | 621 | C | ARG | 880 | 32.874 | 11.647 | 38.889 | 1.00 | 17.11 |
| ATOM | 622 | O | ARG | 880 | 33.459 | 12.631 | 38.400 | 1.00 | 14.72 |
| ATOM | 623 | N | ALA | 881 | 31.609 | 11.376 | 38.531 | 1.00 | 13.92 |
| ATOM | 624 | H | ALA | 881 | 31.183 | 10.585 | 38.930 | 1.00 | 0.00 |
| ATOM | 625 | CA | ALA | 881 | 30.927 | 12.232 | 37.589 | 1.00 | 11.19 |
| ATOM | 626 | CB | ALA | 881 | 29.621 | 11.591 | 37.413 | 1.00 | 10.97 |
| ATOM | 627 | C | ALA | 881 | 30.796 | 13.678 | 38.093 | 1.00 | 13.33 |
| ATOM | 628 | O | ALA | 881 | 30.876 | 14.624 | 37.323 | 1.00 | 13.90 |
| ATOM | 629 | N | LEU | 882 | 30.529 | 13.964 | 39.362 | 1.00 | 11.35 |
| ATOM | 630 | H | LEU | 882 | 30.313 | 13.222 | 39.962 | 1.00 | 0.00 |
| ATOM | 631 | CA | LEU | 882 | 30.415 | 15.326 | 39.851 | 1.00 | 12.11 |
| ATOM | 632 | CB | LEU | 882 | 29.901 | 15.295 | 41.329 | 1.00 | 9.97 |
| ATOM | 633 | CG | LEU | 882 | 29.510 | 16.687 | 41.961 | 1.00 | 9.36 |
| ATOM | 634 | CD1 | LEU | 882 | 28.591 | 17.504 | 41.031 | 1.00 | 8.20 |
| ATOM | 635 | CD2 | LEU | 882 | 28.799 | 16.459 | 43.273 | 1.00 | 11.07 |
| ATOM | 636 | C | LEU | 882 | 31.751 | 16.072 | 39.762 | 1.00 | 16.41 |
| ATOM | 637 | O | LEU | 882 | 31.887 | 17.271 | 39.448 | 1.00 | 18.07 |
| ATOM | 638 | N | MET | 883 | 32.794 | 15.349 | 40.147 | 1.00 | 18.28 |
| ATOM | 639 | H | MET | 883 | 32.621 | 14.475 | 40.551 | 1.00 | 0.00 |
| ATOM | 640 | CA | MET | 883 | 34.178 | 15.858 | 40.115 | 1.00 | 18.40 |
| ATOM | 641 | CB | MET | 883 | 35.187 | 14.751 | 40.713 | 1.00 | 19.62 |
| ATOM | 642 | CG | MET | 883 | 36.591 | 15.198 | 41.010 | 1.00 | 19.11 |
| ATOM | 643 | SD | MET | 883 | 36.840 | 16.792 | 41.953 | 1.00 | 24.05 |
| ATOM | 644 | CE | MET | 883 | 36.551 | 16.061 | 43.566 | 1.00 | 19.82 |
| ATOM | 645 | C | MET | 883 | 34.527 | 16.176 | 38.659 | 1.00 | 18.16 |
| ATOM | 646 | O | MET | 883 | 35.172 | 17.188 | 38.409 | 1.00 | 19.68 |
| ATOM | 647 | N | SER | 884 | 34.153 | 15.358 | 37.653 | 1.00 | 17.17 |
| ATOM | 648 | H | SER | 884 | 33.665 | 14.540 | 37.867 | 1.00 | 0.00 |
| ATOM | 649 | CA | SER | 884 | 34.508 | 15.717 | 36.260 | 1.00 | 16.94 |
| ATOM | 650 | CB | SER | 884 | 34.488 | 14.379 | 35.447 | 1.00 | 17.12 |
| ATOM | 651 | OG | SER | 884 | 33.315 | 14.277 | 34.678 | 1.00 | 27.57 |
| ATOM | 652 | HG | SER | 884 | 33.297 | 13.390 | 34.273 | 1.00 | 0.00 |
| ATOM | 653 | C | SER | 884 | 33.546 | 16.893 | 35.797 | 1.00 | 17.09 |
| ATOM | 654 | O | SER | 884 | 33.918 | 17.784 | 35.020 | 1.00 | 16.43 |
| ATOM | 655 | N | GLU | 885 | 32.331 | 17.020 | 36.322 | 1.00 | 14.31 |
| ATOM | 656 | H | GLU | 885 | 31.952 | 16.289 | 36.862 | 1.00 | 0.00 |
| ATOM | 657 | CA | GLU | 885 | 31.523 | 18.210 | 36.022 | 1.00 | 12.43 |
| ATOM | 658 | CB | GLU | 885 | 30.232 | 17.947 | 36.691 | 1.00 | 12.09 |
| ATOM | 659 | CG | GLU | 885 | 29.550 | 19.249 | 37.182 | 1.00 | 16.28 |
| ATOM | 660 | CD | GLU | 885 | 28.057 | 19.168 | 37.371 | 1.00 | 13.48 |
| ATOM | 661 | OE1 | GLU | 885 | 27.514 | 18.076 | 37.528 | 1.00 | 19.58 |
| ATOM | 662 | OE2 | GLU | 885 | 27.449 | 20.225 | 37.397 | 1.00 | 13.64 |
| ATOM | 663 | C | GLU | 885 | 32.257 | 19.510 | 36.542 | 1.00 | 12.74 |
| ATOM | 664 | O | GLU | 885 | 32.245 | 20.583 | 35.932 | 1.00 | 12.81 |
| ATOM | 665 | N | LEU | 886 | 32.871 | 19.478 | 37.730 | 1.00 | 11.80 |
| ATOM | 666 | H | LEU | 886 | 32.748 | 18.668 | 38.271 | 1.00 | 0.00 |
| ATOM | 667 | CA | LEU | 886 | 33.636 | 20.627 | 38.303 | 1.00 | 10.76 |
| ATOM | 668 | CB | LEU | 886 | 34.110 | 20.251 | 39.730 | 1.00 | 9.62 |
| ATOM | 669 | CG | LEU | 886 | 35.160 | 21.085 | 40.483 | 1.00 | 10.05 |
| ATOM | 670 | CD1 | LEU | 886 | 34.612 | 22.520 | 40.715 | 1.00 | 10.91 |
| ATOM | 671 | CD2 | LEU | 886 | 35.494 | 20.454 | 41.841 | 1.00 | 5.66 |
| ATOM | 672 | C | LEU | 886 | 34.808 | 20.928 | 37.410 | 1.00 | 13.25 |
| ATOM | 673 | O | LEU | 886 | 35.092 | 22.065 | 37.057 | 1.00 | 16.79 |
| ATOM | 674 | N | LYS | 887 | 35.576 | 19.924 | 36.993 | 1.00 | 16.94 |
| ATOM | 675 | H | LYS | 887 | 35.372 | 19.033 | 37.346 | 1.00 | 0.00 |
| ATOM | 676 | CA | LYS | 887 | 36.716 | 20.114 | 36.065 | 1.00 | 17.29 |
| ATOM | 677 | CB | LYS | 887 | 37.357 | 18.763 | 35.866 | 1.00 | 13.73 |
| ATOM | 678 | CG | LYS | 887 | 38.421 | 18.653 | 37.002 | 1.00 | 18.10 |
| ATOM | 679 | CD | LYS | 887 | 38.786 | 17.181 | 36.948 | 1.00 | 19.11 |
| ATOM | 680 | CE | LYS | 887 | 39.817 | 16.685 | 37.967 | 1.00 | 22.23 |
| ATOM | 681 | NZ | LYS | 887 | 39.716 | 15.197 | 38.052 | 1.00 | 23.53 |
| ATOM | 682 | HZ1 | LYS | 887 | 39.789 | 14.820 | 37.086 | 1.00 | 0.00 |
| ATOM | 683 | HZ2 | LYS | 887 | 38.797 | 14.926 | 38.458 | 1.00 | 0.00 |
| ATOM | 684 | HZ3 | LYS | 887 | 40.483 | 14.813 | 38.641 | 1.00 | 0.00 |
| ATOM | 685 | C | LYS | 887 | 36.254 | 20.758 | 34.714 | 1.00 | 17.55 |
| ATOM | 686 | O | LYS | 887 | 36.866 | 21.700 | 34.214 | 1.00 | 17.48 |
| ATOM | 687 | N | ILE | 888 | 35.163 | 20.282 | 34.114 | 1.00 | 19.97 |
| ATOM | 688 | H | ILE | 888 | 34.720 | 19.509 | 34.521 | 1.00 | 0.00 |
| ATOM | 689 | CA | ILE | 888 | 34.605 | 20.838 | 32.855 | 1.00 | 19.61 |
| ATOM | 690 | CB | ILE | 888 | 33.380 | 19.936 | 32.296 | 1.00 | 20.86 |
| ATOM | 691 | CG2 | ILE | 888 | 31.961 | 20.371 | 32.608 | 1.00 | 18.37 |
| ATOM | 692 | CG1 | ILE | 888 | 33.390 | 20.146 | 30.780 | 1.00 | 24.39 |
| ATOM | 693 | CD1 | ILE | 888 | 34.803 | 19.908 | 30.152 | 1.00 | 29.55 |
| ATOM | 694 | C | ILE | 888 | 34.170 | 22.289 | 33.108 | 1.00 | 17.75 |
| ATOM | 695 | O | ILE | 888 | 34.492 | 23.190 | 32.353 | 1.00 | 18.97 |
| ATOM | 696 | N | LEU | 889 | 33.454 | 22.603 | 34.159 | 1.00 | 17.53 |
| ATOM | 697 | H | LEU | 889 | 33.129 | 21.873 | 34.728 | 1.00 | 0.00 |
| ATOM | 698 | CA | LEU | 889 | 33.103 | 24.013 | 34.448 | 1.00 | 16.94 |
| ATOM | 699 | CB | LEU | 889 | 32.214 | 24.097 | 35.730 | 1.00 | 18.05 |
| ATOM | 700 | CG | LEU | 889 | 30.823 | 23.454 | 35.543 | 1.00 | 15.04 |
| ATOM | 701 | CD1 | LEU | 889 | 30.034 | 23.587 | 36.878 | 1.00 | 11.53 |
| ATOM | 702 | CD2 | LEU | 889 | 30.075 | 24.158 | 34.392 | 1.00 | 14.80 |
| ATOM | 703 | C | LEU | 889 | 34.369 | 24.899 | 34.619 | 1.00 | 16.52 |
| ATOM | 704 | O | LEU | 889 | 34.359 | 26.072 | 34.282 | 1.00 | 16.48 |
| ATOM | 705 | N | ILE | 890 | 35.463 | 24.404 | 35.215 | 1.00 | 15.84 |
| ATOM | 706 | H | ILE | 890 | 35.413 | 23.513 | 35.616 | 1.00 | 0.00 |
| ATOM | 707 | CA | ILE | 890 | 36.683 | 25.198 | 35.344 | 1.00 | 14.19 |
| ATOM | 708 | CB | ILE | 890 | 37.671 | 24.447 | 36.180 | 1.00 | 15.23 |
| ATOM | 709 | CG2 | ILE | 890 | 39.053 | 25.168 | 36.045 | 1.00 | 12.95 |
| ATOM | 710 | CG1 | ILE | 890 | 37.155 | 24.348 | 37.634 | 1.00 | 10.26 |
| ATOM | 711 | CD1 | ILE | 890 | 38.026 | 23.458 | 38.549 | 1.00 | 9.98 |
| ATOM | 712 | C | ILE | 890 | 37.209 | 25.385 | 33.931 | 1.00 | 17.35 |
| ATOM | 713 | O | ILE | 890 | 37.602 | 26.472 | 33.516 | 1.00 | 20.68 |
| ATOM | 714 | N | HIS | 891 | 37.164 | 24.341 | 33.106 | 1.00 | 15.37 |
| ATOM | 715 | H | HIS | 891 | 36.842 | 23.484 | 33.448 | 1.00 | 0.00 |
| ATOM | 716 | CA | HIS | 891 | 37.659 | 24.441 | 31.732 | 1.00 | 16.46 |
| ATOM | 717 | C | HIS | 891 | 36.859 | 25.425 | 30.863 | 1.00 | 16.66 |
| ATOM | 718 | O | HIS | 891 | 37.410 | 26.195 | 30.123 | 1.00 | 19.13 |
| ATOM | 719 | CB | HIS | 891 | 37.676 | 22.979 | 31.146 | 1.00 | 15.04 |
| ATOM | 720 | CG | HIS | 891 | 37.818 | 22.967 | 29.697 | 1.00 | 20.20 |
| ATOM | 721 | ND1 | HIS | 891 | 38.941 | 23.354 | 29.041 | 1.00 | 22.20 |
| ATOM | 722 | CE1 | HIS | 891 | 38.561 | 23.506 | 27.733 | 1.00 | 23.52 |
| ATOM | 723 | CD2 | HIS | 891 | 36.752 | 22.870 | 28.782 | 1.00 | 22.96 |
| ATOM | 724 | NE2 | HIS | 891 | 37.247 | 23.233 | 27.587 | 1.00 | 25.26 |
| ATOM | 725 | HE2 | HIS | 891 | 36.777 | 23.226 | 26.723 | 1.00 | 0.00 |
| ATOM | 726 | N | ILE | 892 | 35.552 | 25.352 | 30.846 | 1.00 | 15.69 |
| ATOM | 727 | H | ILE | 892 | 35.186 | 24.637 | 31.403 | 1.00 | 0.00 |
| ATOM | 728 | CA | ILE | 892 | 34.661 | 26.223 | 30.115 | 1.00 | 14.88 |
| ATOM | 729 | CB | ILE | 892 | 33.193 | 25.776 | 30.490 | 1.00 | 12.11 |
| ATOM | 730 | CG2 | ILE | 892 | 32.100 | 26.723 | 30.022 | 1.00 | 12.05 |
| ATOM | 731 | CG1 | ILE | 892 | 32.948 | 24.439 | 29.819 | 1.00 | 10.61 |
| ATOM | 732 | CD1 | ILE | 892 | 31.714 | 23.797 | 30.406 | 1.00 | 7.85 |
| ATOM | 733 | C | ILE | 892 | 34.978 | 27.670 | 30.521 | 1.00 | 15.56 |
| ATOM | 734 | O | ILE | 892 | 35.290 | 28.481 | 29.664 | 1.00 | 15.24 |
| ATOM | 735 | N | GLY | 893 | 35.088 | 28.014 | 31.805 | 1.00 | 16.08 |
| ATOM | 736 | H | GLY | 893 | 35.047 | 27.305 | 32.480 | 1.00 | 0.00 |
| ATOM | 737 | CA | GLY | 893 | 35.307 | 29.417 | 32.221 | 1.00 | 16.21 |
| ATOM | 738 | C | GLY | 893 | 33.989 | 30.264 | 32.137 | 1.00 | 19.13 |
| ATOM | 739 | O | GLY | 893 | 32.873 | 29.830 | 31.835 | 1.00 | 17.08 |
| ATOM | 740 | N | HIS | 894 | 34.030 | 31.557 | 32.430 | 1.00 | 19.53 |
| ATOM | 741 | H | HIS | 894 | 34.887 | 31.900 | 32.761 | 1.00 | 0.00 |
| ATOM | 742 | CA | HIS | 894 | 32.838 | 32.375 | 32.410 | 1.00 | 18.19 |
| ATOM | 743 | C | HIS | 894 | 32.296 | 32.955 | 31.148 | 1.00 | 17.54 |
| ATOM | 744 | O | HIS | 894 | 32.988 | 33.435 | 30.247 | 1.00 | 18.08 |
| ATOM | 745 | CB | HIS | 894 | 33.031 | 33.528 | 33.418 | 1.00 | 23.60 |
| ATOM | 746 | CG | HIS | 894 | 34.368 | 34.226 | 33.342 | 1.00 | 30.85 |
| ATOM | 747 | ND1 | HIS | 894 | 35.442 | 33.961 | 34.160 | 1.00 | 33.58 |
| ATOM | 748 | CE1 | HIS | 894 | 36.449 | 34.865 | 33.915 | 1.00 | 32.32 |
| ATOM | 749 | CD2 | HIS | 894 | 34.777 | 35.341 | 32.560 | 1.00 | 34.72 |
| ATOM | 750 | NE2 | HIS | 894 | 36.040 | 35.728 | 32.903 | 1.00 | 35.30 |
| ATOM | 751 | HE2 | HIS | 894 | 36.665 | 36.286 | 32.380 | 1.00 | 0.00 |
| ATOM | 752 | N | HIS | 895 | 30.995 | 33.079 | 31.155 | 1.00 | 15.30 |
| ATOM | 753 | H | HIS | 895 | 30.499 | 32.572 | 31.845 | 1.00 | 0.00 |
| ATOM | 754 | CA | HIS | 895 | 30.286 | 33.719 | 30.067 | 1.00 | 11.37 |
| ATOM | 755 | C | HIS | 895 | 29.001 | 34.185 | 30.644 | 1.00 | 11.32 |
| ATOM | 756 | O | HIS | 895 | 28.423 | 33.515 | 31.484 | 1.00 | 12.30 |
| ATOM | 757 | CB | HIS | 895 | 30.088 | 32.739 | 28.945 | 1.00 | 13.01 |
| ATOM | 758 | CG | HIS | 895 | 29.383 | 33.418 | 27.817 | 1.00 | 14.49 |
| ATOM | 759 | ND1 | HIS | 895 | 28.037 | 33.549 | 27.707 | 1.00 | 15.06 |
| ATOM | 760 | CE1 | HIS | 895 | 27.751 | 34.327 | 26.638 | 1.00 | 11.35 |
| ATOM | 761 | CD2 | HIS | 895 | 29.933 | 34.146 | 26.764 | 1.00 | 11.75 |
| ATOM | 762 | NE2 | HIS | 895 | 28.915 | 34.678 | 26.079 | 1.00 | 13.10 |
| ATOM | 763 | HE2 | HIS | 895 | 28.964 | 34.964 | 25.135 | 1.00 | 0.00 |
| ATOM | 764 | N | LEU | 896 | 28.459 | 35.299 | 30.252 | 1.00 | 11.00 |
| ATOM | 765 | H | LEU | 896 | 28.839 | 35.763 | 29.469 | 1.00 | 0.00 |
| ATOM | 766 | CA | LEU | 896 | 27.218 | 35.753 | 30.854 | 1.00 | 12.29 |
| ATOM | 767 | CB | LEU | 896 | 26.678 | 36.990 | 30.129 | 1.00 | 13.43 |
| ATOM | 768 | CG | LEU | 896 | 25.398 | 37.605 | 30.652 | 1.00 | 15.65 |
| ATOM | 769 | CD1 | LEU | 896 | 25.798 | 38.282 | 31.953 | 1.00 | 14.94 |
| ATOM | 770 | CD2 | LEU | 896 | 24.678 | 38.452 | 29.564 | 1.00 | 14.75 |
| ATOM | 771 | C | LEU | 896 | 26.142 | 34.702 | 30.758 | 1.00 | 14.99 |
| ATOM | 772 | O | LEU | 896 | 25.265 | 34.526 | 31.619 | 1.00 | 14.38 |
| ATOM | 773 | N | ASN | 897 | 26.134 | 34.038 | 29.620 | 1.00 | 14.77 |
| ATOM | 774 | H | ASN | 897 | 26.809 | 34.230 | 28.944 | 1.00 | 0.00 |
| ATOM | 775 | CA | ASN | 897 | 25.057 | 33.103 | 29.407 | 1.00 | 14.83 |
| ATOM | 776 | CB | ASN | 897 | 24.531 | 33.372 | 27.975 | 1.00 | 13.75 |
| ATOM | 777 | CG | ASN | 897 | 23.935 | 34.750 | 27.799 | 1.00 | 11.29 |
| ATOM | 778 | OD1 | ASN | 897 | 24.419 | 35.465 | 26.959 | 1.00 | 10.51 |
| ATOM | 779 | ND2 | ASN | 897 | 22.921 | 35.217 | 28.499 | 1.00 | 8.71 |
| ATOM | 780 | HD21 | ASN | 897 | 22.484 | 34.674 | 29.188 | 1.00 | 0.00 |
| ATOM | 781 | HD22 | ASN | 897 | 22.590 | 36.115 | 28.319 | 1.00 | 0.00 |
| ATOM | 782 | C | ASN | 897 | 25.262 | 31.620 | 29.665 | 1.00 | 15.28 |
| ATOM | 783 | O | ASN | 897 | 24.543 | 30.744 | 29.159 | 1.00 | 16.01 |
| ATOM | 784 | N | VAL | 898 | 26.171 | 31.327 | 30.552 | 1.00 | 16.22 |
| ATOM | 785 | H | VAL | 898 | 26.682 | 32.049 | 30.980 | 1.00 | 0.00 |
| ATOM | 786 | CA | VAL | 898 | 26.430 | 29.942 | 30.993 | 1.00 | 18.26 |
| ATOM | 787 | CB | VAL | 898 | 27.849 | 29.460 | 30.529 | 1.00 | 19.85 |
| ATOM | 788 | CG1 | VAL | 898 | 28.088 | 28.057 | 31.067 | 1.00 | 24.00 |
| ATOM | 789 | CG2 | VAL | 898 | 27.924 | 29.169 | 29.003 | 1.00 | 17.42 |
| ATOM | 790 | C | VAL | 898 | 26.387 | 30.090 | 32.510 | 1.00 | 18.62 |
| ATOM | 791 | O | VAL | 898 | 26.827 | 31.128 | 33.053 | 1.00 | 18.37 |
| ATOM | 792 | N | VAL | 899 | 25.771 | 29.165 | 33.240 | 1.00 | 17.38 |
| ATOM | 793 | H | VAL | 899 | 25.417 | 28.375 | 32.784 | 1.00 | 0.00 |
| ATOM | 794 | CA | VAL | 899 | 25.757 | 29.312 | 34.718 | 1.00 | 15.88 |
| ATOM | 795 | CB | VAL | 899 | 24.865 | 28.189 | 35.347 | 1.00 | 15.15 |
| ATOM | 796 | CG1 | VAL | 899 | 25.589 | 26.876 | 35.413 | 1.00 | 12.87 |
| ATOM | 797 | CG2 | VAL | 899 | 24.431 | 28.642 | 36.721 | 1.00 | 10.37 |
| ATOM | 798 | C | VAL | 899 | 27.236 | 29.270 | 35.152 | 1.00 | 18.14 |
| ATOM | 799 | O | VAL | 899 | 28.048 | 28.379 | 34.827 | 1.00 | 15.91 |
| ATOM | 800 | N | ASN | 900 | 27.644 | 30.277 | 35.935 | 1.00 | 18.23 |
| ATOM | 801 | H | ASN | 900 | 26.960 | 30.943 | 36.174 | 1.00 | 0.00 |
| ATOM | 802 | CA | ASN | 900 | 29.091 | 30.303 | 36.287 | 1.00 | 18.43 |
| ATOM | 803 | CB | ASN | 900 | 29.685 | 31.828 | 36.189 | 1.00 | 21.36 |
| ATOM | 804 | CG | ASN | 900 | 29.605 | 32.286 | 34.654 | 1.00 | 25.39 |
| ATOM | 805 | OD1 | ASN | 900 | 30.084 | 31.676 | 33.674 | 1.00 | 23.69 |
| ATOM | 806 | ND2 | ASN | 900 | 28.904 | 33.366 | 34.313 | 1.00 | 25.51 |
| ATOM | 807 | HD21 | ASN | 900 | 28.408 | 33.957 | 34.907 | 1.00 | 0.00 |
| ATOM | 808 | HD22 | ASN | 900 | 28.907 | 33.557 | 33.365 | 1.00 | 0.00 |
| ATOM | 809 | C | ASN | 900 | 29.453 | 29.684 | 37.601 | 1.00 | 15.96 |
| ATOM | 810 | O | ASN | 900 | 28.698 | 29.696 | 38.592 | 1.00 | 16.51 |
| ATOM | 811 | N | LEU | 901 | 30.598 | 29.001 | 37.512 | 1.00 | 12.88 |
| ATOM | 812 | H | LEU | 901 | 30.951 | 28.858 | 36.600 | 1.00 | 0.00 |
| ATOM | 813 | CA | LEU | 901 | 31.201 | 28.323 | 38.673 | 1.00 | 14.79 |
| ATOM | 814 | CB | LEU | 901 | 32.287 | 27.329 | 38.234 | 1.00 | 15.66 |
| ATOM | 815 | CG | LEU | 901 | 33.188 | 26.660 | 39.289 | 1.00 | 14.83 |
| ATOM | 816 | CD1 | LEU | 901 | 32.401 | 25.667 | 40.143 | 1.00 | 15.33 |
| ATOM | 817 | CD2 | LEU | 901 | 34.287 | 25.950 | 38.533 | 1.00 | 9.33 |
| ATOM | 818 | C | LEU | 901 | 31.832 | 29.351 | 39.621 | 1.00 | 14.21 |
| ATOM | 819 | O | LEU | 901 | 32.620 | 30.205 | 39.222 | 1.00 | 16.97 |
| ATOM | 820 | N | LEU | 902 | 31.478 | 29.256 | 40.892 | 1.00 | 15.38 |
| ATOM | 821 | H | LEU | 902 | 30.808 | 28.566 | 41.120 | 1.00 | 0.00 |
| ATOM | 822 | CA | LEU | 902 | 31.946 | 30.167 | 41.939 | 1.00 | 15.70 |
| ATOM | 823 | CB | LEU | 902 | 30.715 | 30.723 | 42.726 | 1.00 | 11.96 |
| ATOM | 824 | CG | LEU | 902 | 29.646 | 31.580 | 41.968 | 1.00 | 11.30 |
| ATOM | 825 | CD1 | LEU | 902 | 28.552 | 31.921 | 42.941 | 1.00 | 7.04 |
| ATOM | 826 | CD2 | LEU | 902 | 30.243 | 32.904 | 41.379 | 1.00 | 11.61 |
| ATOM | 827 | C | LEU | 902 | 32.902 | 29.512 | 42.875 | 1.00 | 18.18 |
| ATOM | 828 | O | LEU | 902 | 33.743 | 30.158 | 43.489 | 1.00 | 22.72 |
| ATOM | 829 | N | GLY | 903 | 32.802 | 28.210 | 43.077 | 1.00 | 20.38 |
| ATOM | 830 | H | GLY | 903 | 32.115 | 27.698 | 42.601 | 1.00 | 0.00 |
| ATOM | 831 | CA | GLY | 903 | 33.738 | 27.523 | 44.012 | 1.00 | 17.34 |
| ATOM | 832 | C | GLY | 903 | 33.390 | 26.045 | 44.275 | 1.00 | 18.77 |
| ATOM | 833 | O | GLY | 903 | 32.457 | 25.493 | 43.666 | 1.00 | 17.49 |
| ATOM | 834 | N | ALA | 904 | 34.118 | 25.370 | 45.178 | 1.00 | 15.56 |
| ATOM | 835 | H | ALA | 904 | 34.892 | 25.823 | 45.573 | 1.00 | 0.00 |
| ATOM | 836 | CA | ALA | 904 | 33.798 | 23.975 | 45.469 | 1.00 | 15.00 |
| ATOM | 837 | CB | ALA | 904 | 34.312 | 22.984 | 44.436 | 1.00 | 15.39 |
| ATOM | 838 | C | ALA | 904 | 34.425 | 23.521 | 46.737 | 1.00 | 14.65 |
| ATOM | 839 | O | ALA | 904 | 35.416 | 24.015 | 47.237 | 1.00 | 14.33 |
| ATOM | 840 | N | CYS | 905 | 33.790 | 22.544 | 47.319 | 1.00 | 15.85 |
| ATOM | 841 | H | CYS | 905 | 32.951 | 22.273 | 46.886 | 1.00 | 0.00 |
| ATOM | 842 | CA | CYS | 905 | 34.215 | 21.884 | 48.556 | 1.00 | 16.43 |
| ATOM | 843 | CB | CYS | 905 | 33.128 | 21.898 | 49.591 | 1.00 | 15.29 |
| ATOM | 844 | SG | CYS | 905 | 32.400 | 23.527 | 49.852 | 1.00 | 16.86 |
| ATOM | 845 | C | CYS | 905 | 34.499 | 20.436 | 48.206 | 1.00 | 16.29 |
| ATOM | 846 | O | CYS | 905 | 33.634 | 19.643 | 47.814 | 1.00 | 17.57 |
| ATOM | 847 | N | THR | 906 | 35.788 | 20.288 | 48.040 | 1.00 | 16.30 |
| ATOM | 848 | H | THR | 906 | 36.348 | 21.086 | 48.025 | 1.00 | 0.00 |
| ATOM | 849 | CA | THR | 906 | 36.358 | 19.007 | 47.685 | 1.00 | 17.47 |
| ATOM | 850 | CB | THR | 906 | 37.266 | 19.146 | 46.396 | 1.00 | 15.78 |
| ATOM | 851 | OG1 | THR | 906 | 38.438 | 19.803 | 46.847 | 1.00 | 13.61 |
| ATOM | 852 | HG1 | THR | 906 | 39.097 | 19.744 | 46.143 | 1.00 | 0.00 |
| ATOM | 853 | CG2 | THR | 906 | 36.613 | 19.827 | 45.204 | 1.00 | 9.56 |
| ATOM | 854 | C | THR | 906 | 37.203 | 18.377 | 48.813 | 1.00 | 20.28 |
| ATOM | 855 | O | THR | 906 | 37.547 | 17.206 | 48.744 | 1.00 | 21.94 |
| ATOM | 856 | N | LYS | 907 | 37.596 | 19.121 | 49.871 | 1.00 | 20.91 |
| ATOM | 857 | H | LYS | 907 | 37.302 | 20.047 | 49.884 | 1.00 | 0.00 |
| ATOM | 858 | CA | LYS | 907 | 38.429 | 18.539 | 50.955 | 1.00 | 21.22 |
| ATOM | 859 | CB | LYS | 907 | 38.555 | 19.528 | 52.193 | 1.00 | 21.48 |
| ATOM | 860 | C | LYS | 907 | 37.890 | 17.204 | 51.463 | 1.00 | 23.34 |
| ATOM | 861 | O | LYS | 907 | 36.715 | 17.037 | 51.714 | 1.00 | 24.42 |
| ATOM | 862 | N | PRO | 908 | 38.687 | 16.171 | 51.585 | 1.00 | 24.24 |
| ATOM | 863 | CD | PRO | 908 | 39.839 | 15.888 | 50.752 | 1.00 | 24.78 |
| ATOM | 864 | CA | PRO | 908 | 38.373 | 14.884 | 52.209 | 1.00 | 26.09 |
| ATOM | 865 | CB | PRO | 908 | 39.741 | 14.474 | 52.741 | 1.00 | 26.25 |
| ATOM | 866 | CG | PRO | 908 | 40.706 | 15.315 | 51.895 | 1.00 | 25.56 |
| ATOM | 867 | C | PRO | 908 | 37.278 | 14.548 | 53.204 | 1.00 | 26.87 |
| ATOM | 868 | O | PRO | 908 | 36.467 | 13.596 | 52.986 | 1.00 | 32.72 |
| ATOM | 869 | N | GLY | 909 | 37.177 | 15.283 | 54.296 | 1.00 | 24.01 |
| ATOM | 870 | H | GLY | 909 | 37.685 | 16.106 | 54.434 | 1.00 | 0.00 |
| ATOM | 871 | CA | GLY | 909 | 36.124 | 14.791 | 55.260 | 1.00 | 23.31 |
| ATOM | 872 | C | GLY | 909 | 34.644 | 15.077 | 55.083 | 1.00 | 24.23 |
| ATOM | 873 | O | GLY | 909 | 33.824 | 14.796 | 55.975 | 1.00 | 22.95 |
| ATOM | 874 | N | GLY | 910 | 34.246 | 15.595 | 53.905 | 1.00 | 22.01 |
| ATOM | 875 | H | GLY | 910 | 34.855 | 15.570 | 53.129 | 1.00 | 0.00 |
| ATOM | 876 | CA | GLY | 910 | 32.843 | 15.973 | 53.697 | 1.00 | 17.66 |
| ATOM | 877 | C | GLY | 910 | 32.505 | 15.733 | 52.255 | 1.00 | 18.08 |
| ATOM | 878 | O | GLY | 910 | 33.393 | 15.325 | 51.497 | 1.00 | 18.61 |
| ATOM | 879 | N | PRO | 911 | 31.237 | 15.935 | 51.864 | 1.00 | 18.03 |
| ATOM | 880 | CD | PRO | 911 | 30.076 | 16.397 | 52.683 | 1.00 | 19.10 |
| ATOM | 881 | CA | PRO | 911 | 30.850 | 15.656 | 50.459 | 1.00 | 17.89 |
| ATOM | 882 | CB | PRO | 911 | 29.318 | 15.598 | 50.524 | 1.00 | 17.67 |
| ATOM | 883 | CG | PRO | 911 | 28.970 | 16.599 | 51.627 | 1.00 | 18.65 |
| ATOM | 884 | C | PRO | 911 | 31.367 | 16.645 | 49.386 | 1.00 | 16.98 |
| ATOM | 885 | O | PRO | 911 | 31.665 | 17.829 | 49.624 | 1.00 | 14.66 |
| ATOM | 886 | N | LEU | 912 | 31.598 | 16.130 | 48.185 | 1.00 | 15.56 |
| ATOM | 887 | H | LEU | 912 | 31.661 | 15.161 | 48.143 | 1.00 | 0.00 |
| ATOM | 888 | CA | LEU | 912 | 32.020 | 17.056 | 47.085 | 1.00 | 16.35 |
| ATOM | 889 | CB | LEU | 912 | 32.334 | 16.203 | 45.808 | 1.00 | 15.57 |
| ATOM | 890 | CG | LEU | 912 | 32.528 | 16.952 | 44.485 | 1.00 | 13.48 |
| ATOM | 891 | CD1 | LEU | 912 | 33.643 | 17.931 | 44.700 | 1.00 | 17.88 |
| ATOM | 892 | CD2 | LEU | 912 | 32.882 | 16.001 | 43.346 | 1.00 | 10.85 |
| ATOM | 893 | C | LEU | 912 | 30.822 | 18.078 | 46.801 | 1.00 | 17.94 |
| ATOM | 894 | O | LEU | 912 | 29.604 | 17.732 | 46.739 | 1.00 | 16.04 |
| ATOM | 895 | N | MET | 913 | 31.128 | 19.369 | 46.679 | 1.00 | 16.05 |
| ATOM | 896 | H | MET | 913 | 32.040 | 19.669 | 46.849 | 1.00 | 0.00 |
| ATOM | 897 | CA | MET | 913 | 30.022 | 20.300 | 46.368 | 1.00 | 16.47 |
| ATOM | 898 | CB | MET | 913 | 29.571 | 21.109 | 47.603 | 1.00 | 17.98 |
| ATOM | 899 | CG | MET | 913 | 28.915 | 20.210 | 48.676 | 1.00 | 21.10 |
| ATOM | 900 | SD | MET | 913 | 28.905 | 21.084 | 50.266 | 1.00 | 28.79 |
| ATOM | 901 | CE | MET | 913 | 27.177 | 21.093 | 50.512 | 1.00 | 25.44 |
| ATOM | 902 | C | MET | 913 | 30.509 | 21.275 | 45.311 | 1.00 | 14.82 |
| ATOM | 903 | O | MET | 913 | 31.681 | 21.716 | 45.348 | 1.00 | 13.28 |
| ATOM | 904 | N | VAL | 914 | 29.616 | 21.452 | 44.296 | 1.00 | 14.04 |
| ATOM | 905 | H | VAL | 914 | 28.784 | 20.929 | 44.328 | 1.00 | 0.00 |
| ATOM | 906 | CA | VAL | 914 | 29.887 | 22.367 | 43.157 | 1.00 | 13.28 |
| ATOM | 907 | CB | VAL | 914 | 29.733 | 21.675 | 41.739 | 1.00 | 13.64 |
| ATOM | 908 | CG1 | VAL | 914 | 30.131 | 22.640 | 40.571 | 1.00 | 10.08 |
| ATOM | 909 | CG2 | VAL | 914 | 30.701 | 20.433 | 41.692 | 1.00 | 10.38 |
| ATOM | 910 | C | VAL | 914 | 28.934 | 23.514 | 43.270 | 1.00 | 12.17 |
| ATOM | 911 | O | VAL | 914 | 27.745 | 23.354 | 43.244 | 1.00 | 10.77 |
| ATOM | 912 | N | ILE | 915 | 29.554 | 24.639 | 43.616 | 1.00 | 14.51 |
| ATOM | 913 | H | ILE | 915 | 30.536 | 24.605 | 43.681 | 1.00 | 0.00 |
| ATOM | 914 | CA | ILE | 915 | 28.886 | 25.897 | 43.859 | 1.00 | 12.90 |
| ATOM | 915 | CB | ILE | 915 | 29.615 | 26.593 | 45.065 | 1.00 | 8.58 |
| ATOM | 916 | CG2 | ILE | 915 | 28.789 | 27.846 | 45.400 | 1.00 | 7.68 |
| ATOM | 917 | CG1 | ILE | 915 | 29.668 | 25.707 | 46.353 | 1.00 | 10.26 |
| ATOM | 918 | CD1 | ILE | 915 | 30.926 | 25.985 | 47.182 | 1.00 | 11.87 |
| ATOM | 919 | C | ILE | 915 | 28.835 | 26.771 | 42.651 | 1.00 | 13.14 |
| ATOM | 920 | O | ILE | 915 | 29.872 | 27.060 | 42.062 | 1.00 | 15.44 |
| ATOM | 921 | N | VAL | 916 | 27.663 | 27.213 | 42.264 | 1.00 | 14.36 |
| ATOM | 922 | H | VAL | 916 | 26.872 | 27.005 | 42.803 | 1.00 | 0.00 |
| ATOM | 923 | CA | VAL | 916 | 27.496 | 28.094 | 41.107 | 1.00 | 13.90 |
| ATOM | 924 | CB | VAL | 916 | 26.929 | 27.369 | 39.816 | 1.00 | 12.78 |
| ATOM | 925 | CG1 | VAL | 916 | 27.866 | 26.284 | 39.331 | 1.00 | 12.94 |
| ATOM | 926 | CG2 | VAL | 916 | 25.577 | 26.762 | 40.130 | 1.00 | 7.89 |
| ATOM | 927 | C | VAL | 916 | 26.525 | 29.179 | 41.424 | 1.00 | 15.82 |
| ATOM | 928 | O | VAL | 916 | 25.868 | 29.239 | 42.487 | 1.00 | 14.32 |
| ATOM | 929 | N | GLU | 917 | 26.423 | 30.091 | 40.455 | 1.00 | 19.15 |
| ATOM | 930 | H | GLU | 917 | 27.019 | 30.027 | 39.679 | 1.00 | 0.00 |
| ATOM | 931 | CA | GLU | 917 | 25.453 | 31.220 | 40.582 | 1.00 | 19.33 |
| ATOM | 932 | CB | GLU | 917 | 25.320 | 32.104 | 39.317 | 1.00 | 21.41 |
| ATOM | 933 | CG | GLU | 917 | 26.542 | 33.021 | 39.192 | 1.00 | 28.75 |
| ATOM | 934 | CD | GLU | 917 | 26.676 | 33.567 | 37.740 | 1.00 | 33.02 |
| ATOM | 935 | OE1 | GLU | 917 | 27.186 | 34.695 | 37.606 | 1.00 | 34.78 |
| ATOM | 936 | OE2 | GLU | 917 | 26.292 | 32.873 | 36.779 | 1.00 | 31.14 |
| ATOM | 937 | C | GLU | 917 | 24.034 | 30.797 | 40.805 | 1.00 | 17.15 |
| ATOM | 938 | O | GLU | 917 | 23.517 | 29.899 | 40.150 | 1.00 | 16.18 |
| ATOM | 939 | N | PHE | 918 | 23.403 | 31.551 | 41.689 | 1.00 | 16.63 |
| ATOM | 940 | H | PHE | 918 | 23.923 | 32.260 | 42.113 | 1.00 | 0.00 |
| ATOM | 941 | CA | PHE | 918 | 22.004 | 31.405 | 42.002 | 1.00 | 15.18 |
| ATOM | 942 | CB | PHE | 918 | 21.683 | 31.855 | 43.437 | 1.00 | 13.87 |
| ATOM | 943 | CG | PHE | 918 | 20.240 | 31.621 | 43.701 | 1.00 | 12.92 |
| ATOM | 944 | CD1 | PHE | 918 | 19.718 | 30.329 | 43.699 | 1.00 | 12.03 |
| ATOM | 945 | CD2 | PHE | 918 | 19.391 | 32.708 | 43.925 | 1.00 | 15.12 |
| ATOM | 946 | CE1 | PHE | 918 | 18.375 | 30.093 | 43.918 | 1.00 | 13.89 |
| ATOM | 947 | CE2 | PHE | 918 | 18.029 | 32.482 | 44.149 | 1.00 | 13.17 |
| ATOM | 948 | CZ | PHE | 918 | 17.525 | 31.178 | 44.148 | 1.00 | 13.95 |
| ATOM | 949 | C | PHE | 918 | 21.237 | 32.355 | 41.000 | 1.00 | 17.24 |
| ATOM | 950 | O | PHE | 918 | 21.486 | 33.561 | 40.836 | 1.00 | 19.95 |
| ATOM | 951 | N | CYS | 919 | 20.316 | 31.743 | 40.246 | 1.00 | 17.94 |
| ATOM | 952 | H | CYS | 919 | 20.312 | 30.776 | 40.354 | 1.00 | 0.00 |
| ATOM | 953 | CA | CYS | 919 | 19.386 | 32.329 | 39.260 | 1.00 | 16.08 |
| ATOM | 954 | CB | CYS | 919 | 19.389 | 31.522 | 37.969 | 1.00 | 13.01 |
| ATOM | 955 | SG | CYS | 919 | 20.987 | 31.689 | 37.148 | 1.00 | 19.26 |
| ATOM | 956 | C | CYS | 919 | 17.995 | 32.279 | 39.888 | 1.00 | 17.32 |
| ATOM | 957 | O | CYS | 919 | 17.406 | 31.198 | 40.029 | 1.00 | 15.73 |
| ATOM | 958 | N | LYS | 920 | 17.543 | 33.479 | 40.302 | 1.00 | 19.02 |
| ATOM | 959 | H | LYS | 920 | 18.199 | 34.193 | 40.216 | 1.00 | 0.00 |
| ATOM | 960 | CA | LYS | 920 | 16.236 | 33.797 | 40.955 | 1.00 | 21.12 |
| ATOM | 961 | CB | LYS | 920 | 15.847 | 35.362 | 40.867 | 1.00 | 25.00 |
| ATOM | 962 | CG | LYS | 920 | 15.455 | 36.167 | 42.167 | 1.00 | 26.84 |
| ATOM | 963 | CD | LYS | 920 | 16.382 | 35.868 | 43.455 | 1.00 | 33.07 |
| ATOM | 964 | CE | LYS | 920 | 17.693 | 36.718 | 43.835 | 1.00 | 32.36 |
| ATOM | 965 | NZ | LYS | 920 | 18.880 | 35.949 | 44.285 | 1.00 | 31.79 |
| ATOM | 966 | HZ1 | LYS | 920 | 19.053 | 35.227 | 43.552 | 1.00 | 0.00 |
| ATOM | 967 | HZ2 | LYS | 920 | 18.745 | 35.479 | 45.202 | 1.00 | 0.00 |
| ATOM | 968 | HZ3 | LYS | 920 | 19.698 | 36.593 | 44.321 | 1.00 | 0.00 |
| ATOM | 969 | C | LYS | 920 | 15.018 | 33.079 | 40.454 | 1.00 | 18.84 |
| ATOM | 970 | O | LYS | 920 | 14.351 | 32.385 | 41.157 | 1.00 | 17.15 |
| ATOM | 971 | N | PHE | 921 | 14.712 | 33.210 | 39.225 | 1.00 | 21.82 |
| ATOM | 972 | H | PHE | 921 | 15.348 | 33.648 | 38.627 | 1.00 | 0.00 |
| ATOM | 973 | CA | PHE | 921 | 13.496 | 32.608 | 38.654 | 1.00 | 23.70 |
| ATOM | 974 | CB | PHE | 921 | 13.156 | 33.383 | 37.345 | 1.00 | 23.83 |
| ATOM | 975 | CG | PHE | 921 | 13.024 | 34.774 | 37.752 | 1.00 | 26.04 |
| ATOM | 976 | CD1 | PHE | 921 | 11.934 | 35.191 | 38.528 | 1.00 | 32.38 |
| ATOM | 977 | CD2 | PHE | 921 | 14.007 | 35.681 | 37.401 | 1.00 | 25.14 |
| ATOM | 978 | CE1 | PHE | 921 | 11.858 | 36.544 | 38.944 | 1.00 | 34.87 |
| ATOM | 979 | CE2 | PHE | 921 | 13.927 | 37.007 | 37.808 | 1.00 | 28.94 |
| ATOM | 980 | CZ | PHE | 921 | 12.858 | 37.464 | 38.585 | 1.00 | 30.67 |
| ATOM | 981 | C | PHE | 921 | 13.468 | 31.127 | 38.378 | 1.00 | 23.36 |
| ATOM | 982 | O | PHE | 921 | 12.423 | 30.580 | 38.006 | 1.00 | 22.90 |
| ATOM | 983 | N | GLY | 922 | 14.616 | 30.452 | 38.454 | 1.00 | 22.12 |
| ATOM | 984 | H | GLY | 922 | 15.442 | 30.885 | 38.759 | 1.00 | 0.00 |
| ATOM | 985 | CA | GLY | 922 | 14.536 | 29.012 | 38.146 | 1.00 | 19.67 |
| ATOM | 986 | C | GLY | 922 | 14.527 | 28.615 | 36.669 | 1.00 | 20.65 |
| ATOM | 987 | O | GLY | 922 | 15.021 | 29.348 | 35.795 | 1.00 | 19.51 |
| ATOM | 988 | N | ASN | 923 | 14.017 | 27.421 | 36.333 | 1.00 | 19.03 |
| ATOM | 989 | H | ASN | 923 | 13.581 | 26.894 | 37.027 | 1.00 | 0.00 |
| ATOM | 990 | CA | ASN | 923 | 14.110 | 27.057 | 34.918 | 1.00 | 19.88 |
| ATOM | 991 | CB | ASN | 923 | 14.063 | 25.516 | 34.823 | 1.00 | 23.12 |
| ATOM | 992 | CG | ASN | 923 | 12.692 | 24.989 | 34.694 | 1.00 | 22.71 |
| ATOM | 993 | OD1 | ASN | 923 | 12.097 | 24.850 | 33.620 | 1.00 | 23.53 |
| ATOM | 994 | ND2 | ASN | 923 | 12.120 | 24.657 | 35.813 | 1.00 | 27.07 |
| ATOM | 995 | HD21 | ASN | 923 | 12.516 | 24.723 | 36.702 | 1.00 | 0.00 |
| ATOM | 996 | HD22 | ASN | 923 | 11.218 | 24.316 | 35.697 | 1.00 | 0.00 |
| ATOM | 997 | C | ASN | 923 | 13.134 | 27.752 | 33.981 | 1.00 | 18.78 |
| ATOM | 998 | O | ASN | 923 | 12.011 | 28.092 | 34.331 | 1.00 | 18.41 |
| ATOM | 999 | N | LEU | 924 | 13.559 | 27.899 | 32.740 | 1.00 | 17.52 |
| ATOM | 1000 | H | LEU | 924 | 14.446 | 27.545 | 32.515 | 1.00 | 0.00 |
| ATOM | 1001 | CA | LEU | 924 | 12.807 | 28.630 | 31.763 | 1.00 | 16.30 |
| ATOM | 1002 | CB | LEU | 924 | 13.783 | 28.965 | 30.586 | 1.00 | 14.60 |
| ATOM | 1003 | CG | LEU | 924 | 13.207 | 30.056 | 29.657 | 1.00 | 14.85 |
| ATOM | 1004 | CD1 | LEU | 924 | 13.049 | 31.376 | 30.455 | 1.00 | 12.73 |
| ATOM | 1005 | CD2 | LEU | 924 | 14.117 | 30.265 | 28.479 | 1.00 | 11.02 |
| ATOM | 1006 | C | LEU | 924 | 11.572 | 27.916 | 31.334 | 1.00 | 19.62 |
| ATOM | 1007 | O | LEU | 924 | 10.557 | 28.565 | 31.076 | 1.00 | 16.08 |
| ATOM | 1008 | N | SER | 925 | 11.563 | 26.581 | 31.271 | 1.00 | 21.95 |
| ATOM | 1009 | H | SER | 925 | 12.374 | 26.090 | 31.509 | 1.00 | 0.00 |
| ATOM | 1010 | CA | SER | 925 | 10.263 | 25.946 | 30.858 | 1.00 | 24.99 |
| ATOM | 1011 | CB | SER | 925 | 10.409 | 24.400 | 30.852 | 1.00 | 28.40 |
| ATOM | 1012 | OG | SER | 925 | 9.228 | 23.839 | 30.219 | 1.00 | 32.84 |
| ATOM | 1013 | HG | SER | 925 | 9.324 | 22.881 | 30.168 | 1.00 | 0.00 |
| ATOM | 1014 | C | SER | 925 | 9.046 | 26.335 | 31.770 | 1.00 | 21.30 |
| ATOM | 1015 | O | SER | 925 | 7.976 | 26.837 | 31.424 | 1.00 | 17.59 |
| ATOM | 1016 | N | THR | 926 | 9.274 | 26.091 | 33.030 | 1.00 | 19.92 |
| ATOM | 1017 | H | THR | 926 | 10.113 | 25.626 | 33.236 | 1.00 | 0.00 |
| ATOM | 1018 | CA | THR | 926 | 8.296 | 26.367 | 34.083 | 1.00 | 20.24 |
| ATOM | 1019 | CB | THR | 926 | 8.949 | 25.925 | 35.444 | 1.00 | 19.49 |
| ATOM | 1020 | OG1 | THR | 926 | 9.311 | 24.535 | 35.369 | 1.00 | 19.99 |
| ATOM | 1021 | HG1 | THR | 926 | 9.256 | 24.187 | 36.272 | 1.00 | 0.00 |
| ATOM | 1022 | CG2 | THR | 926 | 7.990 | 26.103 | 36.636 | 1.00 | 19.65 |
| ATOM | 1023 | C | THR | 926 | 7.961 | 27.878 | 34.066 | 1.00 | 20.20 |
| ATOM | 1024 | O | THR | 926 | 6.832 | 28.355 | 34.196 | 1.00 | 21.25 |
| ATOM | 1025 | N | TYR | 927 | 9.001 | 28.678 | 33.967 | 1.00 | 18.43 |
| ATOM | 1026 | H | TYR | 927 | 9.918 | 28.322 | 33.940 | 1.00 | 0.00 |
| ATOM | 1027 | CA | TYR | 927 | 8.769 | 30.099 | 33.957 | 1.00 | 18.77 |
| ATOM | 1028 | CB | TYR | 927 | 10.166 | 30.842 | 33.916 | 1.00 | 18.01 |
| ATOM | 1029 | CG | TYR | 927 | 9.884 | 32.267 | 33.932 | 1.00 | 16.90 |
| ATOM | 1030 | CD1 | TYR | 927 | 9.518 | 32.852 | 35.137 | 1.00 | 18.40 |
| ATOM | 1031 | CE1 | TYR | 927 | 9.202 | 34.198 | 35.163 | 1.00 | 17.87 |
| ATOM | 1032 | CD2 | TYR | 927 | 9.944 | 33.017 | 32.733 | 1.00 | 21.04 |
| ATOM | 1033 | CE2 | TYR | 927 | 9.628 | 34.386 | 32.751 | 1.00 | 19.22 |
| ATOM | 1034 | CZ | TYR | 927 | 9.261 | 34.946 | 33.990 | 1.00 | 21.81 |
| ATOM | 1035 | OH | TYR | 927 | 8.985 | 36.284 | 34.122 | 1.00 | 23.96 |
| ATOM | 1036 | HH | TYR | 927 | 9.073 | 36.753 | 33.289 | 1.00 | 0.00 |
| ATOM | 1037 | C | TYR | 927 | 7.854 | 30.518 | 32.813 | 1.00 | 16.35 |
| ATOM | 1038 | O | TYR | 927 | 6.771 | 30.983 | 33.040 | 1.00 | 16.66 |
| ATOM | 1039 | N | LEU | 928 | 8.200 | 30.378 | 31.582 | 1.00 | 15.19 |
| ATOM | 1040 | H | LEU | 928 | 9.028 | 29.888 | 31.391 | 1.00 | 0.00 |
| ATOM | 1041 | CA | LEU | 928 | 7.295 | 30.836 | 30.475 | 1.00 | 17.09 |
| ATOM | 1042 | CB | LEU | 928 | 7.838 | 30.421 | 29.086 | 1.00 | 13.39 |
| ATOM | 1043 | CG | LEU | 928 | 9.242 | 31.003 | 28.971 | 1.00 | 15.85 |
| ATOM | 1044 | CD1 | LEU | 928 | 9.881 | 30.554 | 27.664 | 1.00 | 19.03 |
| ATOM | 1045 | CD2 | LEU | 928 | 9.180 | 32.559 | 28.982 | 1.00 | 18.38 |
| ATOM | 1046 | C | LEU | 928 | 5.927 | 30.248 | 30.628 | 1.00 | 19.32 |
| ATOM | 1047 | O | LEU | 928 | 4.932 | 30.929 | 30.384 | 1.00 | 18.91 |
| ATOM | 1048 | N | ARG | 929 | 5.823 | 28.973 | 31.085 | 1.00 | 20.90 |
| ATOM | 1049 | H | ARG | 929 | 6.621 | 28.430 | 31.261 | 1.00 | 0.00 |
| ATOM | 1050 | CA | ARG | 929 | 4.463 | 28.374 | 31.216 | 1.00 | 21.61 |
| ATOM | 1051 | CB | ARG | 929 | 4.651 | 26.974 | 31.604 | 1.00 | 24.29 |
| ATOM | 1052 | CG | ARG | 929 | 3.471 | 26.155 | 31.159 | 1.00 | 33.26 |
| ATOM | 1053 | CD | ARG | 929 | 3.901 | 24.683 | 31.081 | 1.00 | 42.70 |
| ATOM | 1054 | NE | ARG | 929 | 4.689 | 24.345 | 32.307 | 1.00 | 51.65 |
| ATOM | 1055 | HE | ARG | 929 | 4.259 | 24.469 | 33.180 | 1.00 | 0.00 |
| ATOM | 1056 | CZ | ARG | 929 | 5.971 | 23.917 | 32.294 | 1.00 | 54.12 |
| ATOM | 1057 | NH1 | ARG | 929 | 6.553 | 23.655 | 33.452 | 1.00 | 54.36 |
| ATOM | 1058 | HH11 | ARG | 929 | 6.062 | 23.780 | 34.311 | 1.00 | 0.00 |
| ATOM | 1059 | HH12 | ARG | 929 | 7.497 | 23.323 | 33.451 | 1.00 | 0.00 |
| ATOM | 1060 | NH2 | ARG | 929 | 6.669 | 23.708 | 31.162 | 1.00 | 58.18 |
| ATOM | 1061 | HH21 | ARG | 929 | 6.248 | 23.872 | 30.271 | 1.00 | 0.00 |
| ATOM | 1062 | HH22 | ARG | 929 | 7.609 | 23.371 | 31.207 | 1.00 | 0.00 |
| ATOM | 1063 | C | ARG | 929 | 3.577 | 29.095 | 32.211 | 1.00 | 22.15 |
| ATOM | 1064 | O | ARG | 929 | 2.364 | 28.988 | 32.264 | 1.00 | 21.68 |
| ATOM | 1065 | N | SER | 930 | 4.203 | 29.805 | 33.129 | 1.00 | 23.87 |
| ATOM | 1066 | H | SER | 930 | 5.183 | 29.797 | 33.155 | 1.00 | 0.00 |
| ATOM | 1067 | CA | SER | 930 | 3.499 | 30.610 | 34.143 | 1.00 | 21.76 |
| ATOM | 1068 | CB | SER | 930 | 4.353 | 30.718 | 35.434 | 1.00 | 19.48 |
| ATOM | 1069 | OG | SER | 930 | 5.470 | 31.537 | 35.145 | 1.00 | 16.46 |
| ATOM | 1070 | HG | SER | 930 | 5.878 | 31.823 | 35.963 | 1.00 | 0.00 |
| ATOM | 1071 | C | SER | 930 | 3.188 | 32.009 | 33.659 | 1.00 | 21.10 |
| ATOM | 1072 | O | SER | 930 | 2.594 | 32.759 | 34.430 | 1.00 | 24.46 |
| ATOM | 1073 | N | LYS | 931 | 3.591 | 32.428 | 32.449 | 1.00 | 19.98 |
| ATOM | 1074 | H | LYS | 931 | 4.177 | 31.828 | 31.947 | 1.00 | 0.00 |
| ATOM | 1075 | CA | LYS | 931 | 3.327 | 33.795 | 31.950 | 1.00 | 19.54 |
| ATOM | 1076 | CB | LYS | 931 | 4.589 | 34.445 | 31.443 | 1.00 | 19.40 |
| ATOM | 1077 | CG | LYS | 931 | 5.710 | 34.572 | 32.526 | 1.00 | 23.38 |
| ATOM | 1078 | CD | LYS | 931 | 5.169 | 34.966 | 33.895 | 1.00 | 20.73 |
| ATOM | 1079 | CE | LYS | 931 | 5.505 | 36.389 | 34.320 | 1.00 | 27.24 |
| ATOM | 1080 | NZ | LYS | 931 | 5.299 | 37.318 | 33.219 | 1.00 | 21.85 |
| ATOM | 1081 | HZ1 | LYS | 931 | 4.396 | 37.090 | 32.763 | 1.00 | 0.00 |
| ATOM | 1082 | HZ2 | LYS | 931 | 6.060 | 37.202 | 32.519 | 1.00 | 0.00 |
| ATOM | 1083 | HZ3 | LYS | 931 | 5.284 | 38.299 | 33.565 | 1.00 | 0.00 |
| ATOM | 1084 | C | LYS | 931 | 2.332 | 33.879 | 30.819 | 1.00 | 20.69 |
| ATOM | 1085 | O | LYS | 931 | 2.184 | 34.889 | 30.126 | 1.00 | 21.58 |
| ATOM | 1086 | N | ARG | 932 | 1.574 | 32.817 | 30.603 | 1.00 | 21.15 |
| ATOM | 1087 | H | ARG | 932 | 1.775 | 32.037 | 31.154 | 1.00 | 0.00 |
| ATOM | 1088 | CA | ARG | 932 | 0.564 | 32.800 | 29.504 | 1.00 | 21.41 |
| ATOM | 1089 | CB | ARG | 932 | −0.096 | 31.405 | 29.460 | 1.00 | 21.89 |
| ATOM | 1090 | CG | ARG | 932 | 0.951 | 30.308 | 29.246 | 1.00 | 19.66 |
| ATOM | 1091 | CD | ARG | 932 | 0.977 | 29.867 | 27.811 | 1.00 | 20.21 |
| ATOM | 1092 | NE | ARG | 932 | 1.889 | 28.731 | 27.703 | 1.00 | 18.67 |
| ATOM | 1093 | HE | ARG | 932 | 2.837 | 28.855 | 27.898 | 1.00 | 0.00 |
| ATOM | 1094 | CZ | ARG | 932 | 1.426 | 27.500 | 27.409 | 1.00 | 21.51 |
| ATOM | 1095 | NH1 | ARG | 932 | 2.317 | 26.500 | 27.292 | 1.00 | 15.25 |
| ATOM | 1096 | HH11 | ARG | 932 | 3.297 | 26.665 | 27.423 | 1.00 | 0.00 |
| ATOM | 1097 | HH12 | ARG | 932 | 1.996 | 25.582 | 27.068 | 1.00 | 0.00 |
| ATOM | 1098 | NH2 | ARG | 932 | 0.084 | 27.230 | 27.281 | 1.00 | 20.54 |
| ATOM | 1099 | HH21 | ARG | 932 | −0.598 | 27.946 | 27.423 | 1.00 | 0.00 |
| ATOM | 1100 | HH22 | ARG | 932 | −0.210 | 26.301 | 27.061 | 1.00 | 0.00 |
| ATOM | 1101 | C | ARG | 932 | −0.491 | 33.898 | 29.669 | 1.00 | 21.72 |
| ATOM | 1102 | O | ARG | 932 | −0.985 | 34.414 | 28.663 | 1.00 | 20.22 |
| ATOM | 1103 | N | ASN | 933 | −0.950 | 34.165 | 30.916 | 1.00 | 22.13 |
| ATOM | 1104 | H | ASN | 933 | −0.559 | 33.644 | 31.652 | 1.00 | 0.00 |
| ATOM | 1105 | CA | ASN | 933 | −1.920 | 35.269 | 31.139 | 1.00 | 23.71 |
| ATOM | 1106 | CB | ASN | 933 | −2.892 | 34.869 | 32.292 | 1.00 | 25.75 |
| ATOM | 1107 | CG | ASN | 933 | −4.243 | 35.633 | 32.354 | 1.00 | 29.48 |
| ATOM | 1108 | OD1 | ASN | 933 | −4.910 | 35.873 | 31.356 | 1.00 | 30.03 |
| ATOM | 1109 | ND2 | ASN | 933 | −4.831 | 35.934 | 33.499 | 1.00 | 29.53 |
| ATOM | 1110 | HD21 | ASN | 933 | −4.507 | 35.730 | 34.394 | 1.00 | 0.00 |
| ATOM | 1111 | HD22 | ASN | 933 | −5.672 | 36.419 | 33.415 | 1.00 | 0.00 |
| ATOM | 1112 | C | ASN | 933 | −1.163 | 36.617 | 31.429 | 1.00 | 24.80 |
| ATOM | 1113 | O | ASN | 933 | −1.736 | 37.649 | 31.816 | 1.00 | 23.64 |
| ATOM | 1114 | N | GLU | 934 | 0.145 | 36.654 | 31.078 | 1.00 | 24.28 |
| ATOM | 1115 | H | GLU | 934 | 0.517 | 35.816 | 30.745 | 1.00 | 0.00 |
| ATOM | 1116 | CA | GLU | 934 | 1.066 | 37.815 | 31.231 | 1.00 | 23.94 |
| ATOM | 1117 | CB | GLU | 934 | 1.852 | 37.665 | 32.457 | 1.00 | 25.15 |
| ATOM | 1118 | CG | GLU | 934 | 0.991 | 37.875 | 33.672 | 1.00 | 31.71 |
| ATOM | 1119 | CD | GLU | 934 | 1.782 | 37.302 | 34.833 | 1.00 | 35.16 |
| ATOM | 1120 | OE1 | GLU | 934 | 1.683 | 36.109 | 35.099 | 1.00 | 40.39 |
| ATOM | 1121 | OE2 | GLU | 934 | 2.560 | 38.037 | 35.425 | 1.00 | 38.33 |
| ATOM | 1122 | C | GLU | 934 | 2.060 | 37.987 | 30.095 | 1.00 | 24.50 |
| ATOM | 1123 | O | GLU | 934 | 3.228 | 38.328 | 30.286 | 1.00 | 25.85 |
| ATOM | 1124 | N | PHE | 935 | 1.570 | 37.802 | 28.888 | 1.00 | 22.27 |
| ATOM | 1125 | H | PHE | 935 | 0.613 | 37.603 | 28.833 | 1.00 | 0.00 |
| ATOM | 1126 | CA | PHE | 935 | 2.339 | 37.961 | 27.661 | 1.00 | 22.45 |
| ATOM | 1127 | CB | PHE | 935 | 2.680 | 36.566 | 27.007 | 1.00 | 23.10 |
| ATOM | 1128 | CG | PHE | 935 | 3.270 | 36.648 | 25.610 | 1.00 | 18.95 |
| ATOM | 1129 | CD1 | PHE | 935 | 2.449 | 36.671 | 24.491 | 1.00 | 15.94 |
| ATOM | 1130 | CD2 | PHE | 935 | 4.663 | 36.743 | 25.444 | 1.00 | 17.90 |
| ATOM | 1131 | CE1 | PHE | 935 | 3.016 | 36.788 | 23.226 | 1.00 | 19.40 |
| ATOM | 1132 | CE2 | PHE | 935 | 5.255 | 36.859 | 24.178 | 1.00 | 18.94 |
| ATOM | 1133 | CZ | PHE | 935 | 4.415 | 36.880 | 23.072 | 1.00 | 21.33 |
| ATOM | 1134 | C | PHE | 935 | 1.535 | 38.813 | 26.647 | 1.00 | 25.01 |
| ATOM | 1135 | O | PHE | 935 | 0.306 | 38.791 | 26.632 | 1.00 | 24.55 |
| ATOM | 1136 | N | VAL | 936 | 2.217 | 39.616 | 25.828 | 1.00 | 26.79 |
| ATOM | 1137 | H | VAL | 936 | 3.182 | 39.671 | 26.011 | 1.00 | 0.00 |
| ATOM | 1138 | CA | VAL | 936 | 1.686 | 40.502 | 24.769 | 1.00 | 28.80 |
| ATOM | 1139 | CB | VAL | 936 | 1.480 | 41.996 | 25.145 | 1.00 | 33.06 |
| ATOM | 1140 | CG1 | VAL | 936 | 0.094 | 42.038 | 25.685 | 1.00 | 32.64 |
| ATOM | 1141 | CG2 | VAL | 936 | 2.515 | 42.596 | 26.178 | 1.00 | 32.57 |
| ATOM | 1142 | C | VAL | 936 | 2.785 | 40.559 | 23.767 | 1.00 | 29.12 |
| ATOM | 1143 | O | VAL | 936 | 3.925 | 40.723 | 24.188 | 1.00 | 25.43 |
| ATOM | 1144 | N | PRO | 937 | 2.517 | 40.385 | 22.470 | 1.00 | 32.27 |
| ATOM | 1145 | CD | PRO | 937 | 1.205 | 40.192 | 21.884 | 1.00 | 33.79 |
| ATOM | 1146 | CA | PRO | 937 | 3.555 | 40.277 | 21.414 | 1.00 | 34.65 |
| ATOM | 1147 | CB | PRO | 937 | 2.845 | 39.960 | 20.128 | 1.00 | 31.59 |
| ATOM | 1148 | CG | PRO | 937 | 1.653 | 39.292 | 20.727 | 1.00 | 34.38 |
| ATOM | 1149 | C | PRO | 937 | 4.372 | 41.552 | 21.257 | 1.00 | 38.78 |
| ATOM | 1150 | O | PRO | 937 | 5.600 | 41.547 | 21.176 | 1.00 | 39.37 |
| ATOM | 1151 | N | TYR | 938 | 3.690 | 42.666 | 21.168 | 1.00 | 43.58 |
| ATOM | 1152 | H | TYR | 938 | 2.738 | 42.685 | 21.389 | 1.00 | 0.00 |
| ATOM | 1153 | CA | TYR | 938 | 4.322 | 44.003 | 21.023 | 1.00 | 50.41 |
| ATOM | 1154 | CB | TYR | 938 | 3.788 | 44.608 | 19.666 | 1.00 | 49.04 |
| ATOM | 1155 | C | TYR | 938 | 3.735 | 44.566 | 22.358 | 1.00 | 55.69 |
| ATOM | 1156 | O | TYR | 938 | 2.498 | 44.549 | 22.549 | 1.00 | 56.83 |
| ATOM | 1158 | CB | ASP | 998 | 6.508 | 42.476 | 35.275 | 1.00 | 28.93 |
| ATOM | 1159 | C | ASP | 998 | 5.213 | 41.987 | 33.192 | 1.00 | 29.56 |
| ATOM | 1160 | O | ASP | 998 | 5.538 | 40.841 | 33.371 | 1.00 | 30.30 |
| ATOM | 1161 | HT1 | ASP | 998 | 3.720 | 42.227 | 35.234 | 1.00 | 0.00 |
| ATOM | 1162 | HT2 | ASP | 998 | 3.345 | 43.678 | 34.450 | 1.00 | 0.00 |
| ATOM | 1163 | N | ASP | 998 | 4.068 | 43.184 | 35.007 | 1.00 | 31.56 |
| ATOM | 1164 | HT3 | ASP | 998 | 4.211 | 43.693 | 35.903 | 1.00 | 0.00 |
| ATOM | 1165 | CA | ASP | 998 | 5.377 | 43.016 | 34.308 | 1.00 | 30.69 |
| ATOM | 1166 | N | PHE | 999 | 4.518 | 42.282 | 32.122 | 1.00 | 28.82 |
| ATOM | 1167 | H | PHE | 999 | 4.230 | 43.191 | 31.918 | 1.00 | 0.00 |
| ATOM | 1168 | CA | PHE | 999 | 4.350 | 41.306 | 31.044 | 1.00 | 26.91 |
| ATOM | 1169 | CB | PHE | 999 | 3.347 | 41.914 | 30.059 | 1.00 | 28.34 |
| ATOM | 1170 | CG | PHE | 999 | 1.915 | 41.744 | 30.372 | 1.00 | 34.01 |
| ATOM | 1171 | CD1 | PHE | 999 | 1.059 | 41.324 | 29.301 | 1.00 | 36.82 |
| ATOM | 1172 | CD2 | PHE | 999 | 1.355 | 41.990 | 31.641 | 1.00 | 33.75 |
| ATOM | 1173 | CE1 | PHE | 999 | −0.342 | 41.118 | 29.478 | 1.00 | 35.71 |
| ATOM | 1174 | CE2 | PHE | 999 | −0.042 | 41.795 | 31.834 | 1.00 | 35.51 |
| ATOM | 1175 | CZ | PHE | 999 | −0.882 | 41.370 | 30.755 | 1.00 | 36.42 |
| ATOM | 1176 | C | PHE | 999 | 5.613 | 40.852 | 30.242 | 1.00 | 27.29 |
| ATOM | 1177 | O | PHE | 999 | 6.618 | 41.578 | 30.096 | 1.00 | 25.99 |
| ATOM | 1178 | N | LEU | 1000 | 5.615 | 39.605 | 29.754 | 1.00 | 24.56 |
| ATOM | 1179 | H | LEU | 1000 | 4.908 | 38.993 | 30.029 | 1.00 | 0.00 |
| ATOM | 1180 | CA | LEU | 1000 | 6.674 | 39.157 | 28.855 | 1.00 | 21.27 |
| ATOM | 1181 | CB | LEU | 1000 | 6.830 | 37.663 | 28.689 | 1.00 | 21.02 |
| ATOM | 1182 | CG | LEU | 1000 | 7.483 | 36.797 | 29.724 | 1.00 | 20.29 |
| ATOM | 1183 | CD1 | LEU | 1000 | 7.090 | 35.427 | 29.279 | 1.00 | 19.43 |
| ATOM | 1184 | CD2 | LEU | 1000 | 9.018 | 36.957 | 29.858 | 1.00 | 17.94 |
| ATOM | 1185 | C | LEU | 1000 | 6.145 | 39.607 | 27.520 | 1.00 | 21.17 |
| ATOM | 1186 | O | LEU | 1000 | 4.938 | 39.655 | 27.267 | 1.00 | 21.34 |
| ATOM | 1187 | N | THR | 1001 | 7.028 | 39.804 | 26.587 | 1.00 | 21.60 |
| ATOM | 1188 | H | THR | 1001 | 7.951 | 39.585 | 26.783 | 1.00 | 0.00 |
| ATOM | 1189 | CA | THR | 1001 | 6.660 | 40.281 | 25.244 | 1.00 | 21.24 |
| ATOM | 1190 | CB | THR | 1001 | 6.984 | 41.813 | 25.030 | 1.00 | 20.92 |
| ATOM | 1191 | OG1 | THR | 1001 | 8.380 | 41.964 | 25.197 | 1.00 | 23.92 |
| ATOM | 1192 | HG1 | THR | 1001 | 8.544 | 42.915 | 25.154 | 1.00 | 0.00 |
| ATOM | 1193 | CG2 | THR | 1001 | 6.377 | 42.768 | 26.028 | 1.00 | 17.87 |
| ATOM | 1194 | C | THR | 1001 | 7.469 | 39.499 | 24.236 | 1.00 | 20.92 |
| ATOM | 1195 | O | THR | 1001 | 8.404 | 38.767 | 24.578 | 1.00 | 21.09 |
| ATOM | 1196 | N | LEU | 1002 | 7.193 | 39.626 | 22.949 | 1.00 | 21.67 |
| ATOM | 1197 | H | LEU | 1002 | 6.481 | 40.244 | 22.671 | 1.00 | 0.00 |
| ATOM | 1198 | CA | LEU | 1002 | 7.993 | 38.899 | 21.940 | 1.00 | 22.89 |
| ATOM | 1199 | CB | LEU | 1002 | 7.272 | 39.248 | 20.665 | 1.00 | 22.48 |
| ATOM | 1200 | CG | LEU | 1002 | 7.660 | 38.493 | 19.446 | 1.00 | 24.55 |
| ATOM | 1201 | CD1 | LEU | 1002 | 7.643 | 36.963 | 19.691 | 1.00 | 25.92 |
| ATOM | 1202 | CD2 | LEU | 1002 | 6.709 | 39.009 | 18.338 | 1.00 | 26.20 |
| ATOM | 1203 | C | LEU | 1002 | 9.508 | 39.296 | 21.986 | 1.00 | 23.74 |
| ATOM | 1204 | O | LEU | 1002 | 10.487 | 38.551 | 21.727 | 1.00 | 24.39 |
| ATOM | 1205 | N | GLU | 1003 | 9.775 | 40.575 | 22.341 | 1.00 | 24.14 |
| ATOM | 1206 | H | GLU | 1003 | 9.015 | 41.192 | 22.461 | 1.00 | 0.00 |
| ATOM | 1207 | CA | GLU | 1003 | 11.164 | 41.026 | 22.446 | 1.00 | 20.97 |
| ATOM | 1208 | CB | GLU | 1003 | 11.172 | 42.419 | 22.965 | 1.00 | 23.17 |
| ATOM | 1209 | CG | GLU | 1003 | 12.572 | 42.973 | 22.557 | 1.00 | 29.21 |
| ATOM | 1210 | CD | GLU | 1003 | 12.841 | 44.378 | 23.071 | 1.00 | 32.91 |
| ATOM | 1211 | OE1 | GLU | 1003 | 11.881 | 45.027 | 23.520 | 1.00 | 34.92 |
| ATOM | 1212 | OE2 | GLU | 1003 | 14.019 | 44.784 | 23.050 | 1.00 | 35.11 |
| ATOM | 1213 | C | GLU | 1003 | 11.899 | 40.117 | 23.451 | 1.00 | 20.17 |
| ATOM | 1214 | O | GLU | 1003 | 12.997 | 39.631 | 23.255 | 1.00 | 20.77 |
| ATOM | 1215 | N | HIS | 1004 | 11.297 | 39.896 | 24.608 | 1.00 | 18.66 |
| ATOM | 1216 | H | HIS | 1004 | 10.423 | 40.318 | 24.747 | 1.00 | 0.00 |
| ATOM | 1217 | CA | HIS | 1004 | 11.859 | 39.018 | 25.650 | 1.00 | 16.11 |
| ATOM | 1218 | C | HIS | 1004 | 12.115 | 37.636 | 25.104 | 1.00 | 14.31 |
| ATOM | 1219 | O | HIS | 1004 | 13.127 | 37.087 | 25.410 | 1.00 | 16.01 |
| ATOM | 1220 | CB | HIS | 1004 | 10.909 | 38.800 | 26.795 | 1.00 | 15.39 |
| ATOM | 1221 | CG | HIS | 1004 | 10.633 | 39.992 | 27.549 | 1.00 | 14.24 |
| ATOM | 1222 | ND1 | HIS | 1004 | 11.258 | 40.313 | 28.691 | 1.00 | 17.83 |
| ATOM | 1223 | HD1 | HIS | 1004 | 11.884 | 39.748 | 29.195 | 1.00 | 0.00 |
| ATOM | 1224 | CD2 | HIS | 1004 | 9.706 | 40.957 | 27.280 | 1.00 | 13.74 |
| ATOM | 1225 | NE2 | HIS | 1004 | 9.784 | 41.854 | 28.262 | 1.00 | 17.11 |
| ATOM | 1226 | CE1 | HIS | 1004 | 10.731 | 41.479 | 29.157 | 1.00 | 14.94 |
| ATOM | 1227 | N | LEU | 1005 | 11.221 | 37.050 | 24.322 | 1.00 | 13.63 |
| ATOM | 1228 | H | LEU | 1005 | 10.409 | 37.573 | 24.143 | 1.00 | 0.00 |
| ATOM | 1229 | CA | LEU | 1005 | 11.342 | 35.681 | 23.748 | 1.00 | 14.44 |
| ATOM | 1230 | CB | LEU | 1005 | 10.024 | 35.326 | 23.049 | 1.00 | 12.32 |
| ATOM | 1231 | CG | LEU | 1005 | 8.866 | 34.721 | 23.879 | 1.00 | 16.30 |
| ATOM | 1232 | CD1 | LEU | 1005 | 8.813 | 35.267 | 25.291 | 1.00 | 17.07 |
| ATOM | 1233 | CD2 | LEU | 1005 | 7.657 | 34.768 | 22.969 | 1.00 | 12.24 |
| ATOM | 1234 | C | LEU | 1005 | 12.502 | 35.539 | 22.774 | 1.00 | 15.79 |
| ATOM | 1235 | O | LEU | 1005 | 13.263 | 34.555 | 22.849 | 1.00 | 16.62 |
| ATOM | 1236 | N | ILE | 1006 | 12.668 | 36.540 | 21.861 | 1.00 | 15.94 |
| ATOM | 1237 | H | ILE | 1006 | 11.983 | 37.238 | 21.840 | 1.00 | 0.00 |
| ATOM | 1238 | CA | ILE | 1006 | 13.817 | 36.556 | 20.872 | 1.00 | 14.92 |
| ATOM | 1239 | CB | ILE | 1006 | 13.694 | 37.689 | 19.774 | 1.00 | 16.71 |
| ATOM | 1240 | CG2 | ILE | 1006 | 14.855 | 37.555 | 18.700 | 1.00 | 13.62 |
| ATOM | 1241 | CG1 | ILE | 1006 | 12.255 | 37.629 | 19.200 | 1.00 | 17.08 |
| ATOM | 1242 | CD1 | ILE | 1006 | 11.852 | 38.969 | 18.499 | 1.00 | 20.74 |
| ATOM | 1243 | C | ILE | 1006 | 15.130 | 36.860 | 21.680 | 1.00 | 14.76 |
| ATOM | 1244 | O | ILE | 1006 | 16.207 | 36.399 | 21.349 | 1.00 | 13.80 |
| ATOM | 1245 | N | CYS | 1007 | 15.041 | 37.795 | 22.622 | 1.00 | 12.39 |
| ATOM | 1246 | H | CYS | 1007 | 14.192 | 38.275 | 22.680 | 1.00 | 0.00 |
| ATOM | 1247 | CA | CYS | 1007 | 16.130 | 38.129 | 23.460 | 1.00 | 14.00 |
| ATOM | 1248 | CB | CYS | 1007 | 15.675 | 39.211 | 24.449 | 1.00 | 15.16 |
| ATOM | 1249 | SG | CYS | 1007 | 16.958 | 39.608 | 25.668 | 1.00 | 20.35 |
| ATOM | 1250 | C | CYS | 1007 | 16.609 | 36.884 | 24.210 | 1.00 | 13.20 |
| ATOM | 1251 | O | CYS | 1007 | 17.807 | 36.574 | 24.256 | 1.00 | 14.56 |
| ATOM | 1252 | N | TYR | 1008 | 15.745 | 36.128 | 24.835 | 1.00 | 13.12 |
| ATOM | 1253 | H | TYR | 1008 | 14.802 | 36.387 | 24.869 | 1.00 | 0.00 |
| ATOM | 1254 | CA | TYR | 1008 | 16.201 | 34.941 | 25.541 | 1.00 | 12.69 |
| ATOM | 1255 | CB | TYR | 1008 | 15.030 | 34.283 | 26.242 | 1.00 | 14.59 |
| ATOM | 1256 | CG | TYR | 1008 | 14.383 | 35.068 | 27.314 | 1.00 | 16.09 |
| ATOM | 1257 | CD1 | TYR | 1008 | 13.052 | 34.764 | 27.578 | 1.00 | 16.62 |
| ATOM | 1258 | CE1 | TYR | 1008 | 12.331 | 35.418 | 28.592 | 1.00 | 15.46 |
| ATOM | 1259 | CD2 | TYR | 1008 | 15.032 | 36.059 | 28.066 | 1.00 | 19.40 |
| ATOM | 1260 | CE2 | TYR | 1008 | 14.312 | 36.727 | 29.089 | 1.00 | 18.40 |
| ATOM | 1261 | CZ | TYR | 1008 | 12.979 | 36.382 | 29.335 | 1.00 | 17.27 |
| ATOM | 1262 | OH | TYR | 1008 | 12.326 | 36.864 | 30.438 | 1.00 | 21.02 |
| ATOM | 1263 | HH | TYR | 1008 | 11.463 | 36.464 | 30.538 | 1.00 | 0.00 |
| ATOM | 1264 | C | TYR | 1008 | 16.820 | 33.942 | 24.546 | 1.00 | 14.66 |
| ATOM | 1265 | O | TYR | 1008 | 17.866 | 33.324 | 24.832 | 1.00 | 13.22 |
| ATOM | 1266 | N | SER | 1009 | 16.216 | 33.757 | 23.360 | 1.00 | 13.65 |
| ATOM | 1267 | H | SER | 1009 | 15.311 | 34.104 | 23.199 | 1.00 | 0.00 |
| ATOM | 1268 | CA | SER | 1009 | 16.810 | 32.870 | 22.360 | 1.00 | 14.29 |
| ATOM | 1269 | CB | SER | 1009 | 16.000 | 32.942 | 21.101 | 1.00 | 15.10 |
| ATOM | 1270 | OG | SER | 1009 | 14.683 | 32.554 | 21.382 | 1.00 | 17.75 |
| ATOM | 1271 | HG | SER | 1009 | 14.159 | 32.636 | 20.579 | 1.00 | 0.00 |
| ATOM | 1272 | C | SER | 1009 | 18.256 | 33.180 | 21.943 | 1.00 | 11.87 |
| ATOM | 1273 | O | SER | 1009 | 19.136 | 32.350 | 21.682 | 1.00 | 11.61 |
| ATOM | 1274 | N | PHE | 1010 | 18.457 | 34.464 | 21.627 | 1.00 | 14.47 |
| ATOM | 1275 | H | PHE | 1010 | 17.676 | 35.062 | 21.655 | 1.00 | 0.00 |
| ATOM | 1276 | CA | PHE | 1010 | 19.757 | 35.033 | 21.217 | 1.00 | 11.35 |
| ATOM | 1277 | CB | PHE | 1010 | 19.608 | 36.608 | 20.969 | 1.00 | 15.78 |
| ATOM | 1278 | CG | PHE | 1010 | 20.879 | 37.386 | 20.803 | 1.00 | 16.59 |
| ATOM | 1279 | CD1 | PHE | 1010 | 21.311 | 38.232 | 21.856 | 1.00 | 15.51 |
| ATOM | 1280 | CD2 | PHE | 1010 | 21.708 | 37.220 | 19.631 | 1.00 | 12.86 |
| ATOM | 1281 | CE1 | PHE | 1010 | 22.582 | 38.869 | 21.704 | 1.00 | 16.04 |
| ATOM | 1282 | CE2 | PHE | 1010 | 22.953 | 37.865 | 19.495 | 1.00 | 13.44 |
| ATOM | 1283 | CZ | PHE | 1010 | 23.385 | 38.692 | 20.547 | 1.00 | 13.22 |
| ATOM | 1284 | C | PHE | 1010 | 20.699 | 34.773 | 22.341 | 1.00 | 10.56 |
| ATOM | 1285 | O | PHE | 1010 | 21.817 | 34.307 | 22.140 | 1.00 | 13.25 |
| ATOM | 1286 | N | GLN | 1011 | 20.329 | 35.116 | 23.576 | 1.00 | 11.15 |
| ATOM | 1287 | H | GLN | 1011 | 19.474 | 35.569 | 23.706 | 1.00 | 0.00 |
| ATOM | 1288 | CA | GLN | 1011 | 21.226 | 34.816 | 24.708 | 1.00 | 10.82 |
| ATOM | 1289 | CB | GLN | 1011 | 20.596 | 35.242 | 26.008 | 1.00 | 11.24 |
| ATOM | 1290 | CG | GLN | 1011 | 20.531 | 36.746 | 26.131 | 1.00 | 11.40 |
| ATOM | 1291 | CD | GLN | 1011 | 19.957 | 36.941 | 27.517 | 1.00 | 16.67 |
| ATOM | 1292 | OE1 | GLN | 1011 | 20.661 | 36.722 | 28.531 | 1.00 | 16.41 |
| ATOM | 1293 | NE2 | GLN | 1011 | 18.669 | 37.339 | 27.637 | 1.00 | 15.31 |
| ATOM | 1294 | HE21 | GLN | 1011 | 18.092 | 37.508 | 26.874 | 1.00 | 0.00 |
| ATOM | 1295 | HE22 | GLN | 1011 | 18.327 | 37.462 | 28.539 | 1.00 | 0.00 |
| ATOM | 1296 | C | GLN | 1011 | 21.578 | 33.309 | 24.809 | 1.00 | 13.27 |
| ATOM | 1297 | O | GLN | 1011 | 22.752 | 32.994 | 25.055 | 1.00 | 17.22 |
| ATOM | 1298 | N | VAL | 1012 | 20.631 | 32.354 | 24.685 | 1.00 | 12.07 |
| ATOM | 1299 | H | VAL | 1012 | 19.693 | 32.625 | 24.636 | 1.00 | 0.00 |
| ATOM | 1300 | CA | VAL | 1012 | 21.035 | 30.940 | 24.725 | 1.00 | 13.58 |
| ATOM | 1301 | CB | VAL | 1012 | 19.847 | 29.993 | 24.671 | 1.00 | 15.17 |
| ATOM | 1302 | CG1 | VAL | 1012 | 20.337 | 28.529 | 24.615 | 1.00 | 9.53 |
| ATOM | 1303 | CG2 | VAL | 1012 | 18.954 | 30.252 | 25.922 | 1.00 | 10.35 |
| ATOM | 1304 | C | VAL | 1012 | 21.912 | 30.693 | 23.493 | 1.00 | 14.71 |
| ATOM | 1305 | O | VAL | 1012 | 22.939 | 30.016 | 23.597 | 1.00 | 18.45 |
| ATOM | 1306 | N | ALA | 1013 | 21.622 | 31.180 | 22.281 | 1.00 | 15.02 |
| ATOM | 1307 | H | ALA | 1013 | 20.796 | 31.669 | 22.121 | 1.00 | 0.00 |
| ATOM | 1308 | CA | ALA | 1013 | 22.630 | 30.916 | 21.175 | 1.00 | 15.09 |
| ATOM | 1309 | CB | ALA | 1013 | 22.144 | 31.479 | 19.809 | 1.00 | 10.33 |
| ATOM | 1310 | C | ALA | 1013 | 24.043 | 31.515 | 21.488 | 1.00 | 13.18 |
| ATOM | 1311 | O | ALA | 1013 | 25.075 | 30.997 | 21.077 | 1.00 | 13.32 |
| ATOM | 1312 | N | LYS | 1014 | 24.184 | 32.619 | 22.216 | 1.00 | 13.23 |
| ATOM | 1313 | H | LYS | 1014 | 23.370 | 33.118 | 22.432 | 1.00 | 0.00 |
| ATOM | 1314 | CA | LYS | 1014 | 25.517 | 33.162 | 22.618 | 1.00 | 13.09 |
| ATOM | 1315 | CB | LYS | 1014 | 25.363 | 34.565 | 23.328 | 1.00 | 15.20 |
| ATOM | 1316 | CG | LYS | 1014 | 24.906 | 35.746 | 22.453 | 1.00 | 15.92 |
| ATOM | 1317 | CD | LYS | 1014 | 26.038 | 36.236 | 21.582 | 1.00 | 20.55 |
| ATOM | 1318 | CE | LYS | 1014 | 26.690 | 37.427 | 22.302 | 1.00 | 25.41 |
| ATOM | 1319 | NZ | LYS | 1014 | 27.734 | 38.046 | 21.449 | 1.00 | 20.55 |
| ATOM | 1320 | HZ1 | LYS | 1014 | 27.329 | 38.252 | 20.519 | 1.00 | 0.00 |
| ATOM | 1321 | HZ2 | LYS | 1014 | 28.534 | 37.387 | 21.352 | 1.00 | 0.00 |
| ATOM | 1322 | HZ3 | LYS | 1014 | 28.062 | 38.918 | 21.903 | 1.00 | 0.00 |
| ATOM | 1323 | C | LYS | 1014 | 26.269 | 32.213 | 23.593 | 1.00 | 12.73 |
| ATOM | 1324 | O | LYS | 1014 | 27.493 | 32.026 | 23.597 | 1.00 | 13.18 |
| ATOM | 1325 | N | GLY | 1015 | 25.516 | 31.657 | 24.540 | 1.00 | 13.83 |
| ATOM | 1326 | H | GLY | 1015 | 24.595 | 31.996 | 24.613 | 1.00 | 0.00 |
| ATOM | 1327 | CA | GLY | 1015 | 26.026 | 30.702 | 25.541 | 1.00 | 11.61 |
| ATOM | 1328 | C | GLY | 1015 | 26.475 | 29.424 | 24.836 | 1.00 | 12.05 |
| ATOM | 1329 | O | GLY | 1015 | 27.496 | 28.908 | 25.196 | 1.00 | 11.48 |
| ATOM | 1330 | N | MET | 1016 | 25.719 | 28.893 | 23.876 | 1.00 | 10.52 |
| ATOM | 1331 | H | MET | 1016 | 24.855 | 29.338 | 23.741 | 1.00 | 0.00 |
| ATOM | 1332 | CA | MET | 1016 | 26.034 | 27.689 | 23.089 | 1.00 | 12.01 |
| ATOM | 1333 | CB | MET | 1016 | 24.672 | 27.363 | 22.400 | 1.00 | 9.99 |
| ATOM | 1334 | CG | MET | 1016 | 23.661 | 26.614 | 23.305 | 1.00 | 10.09 |
| ATOM | 1335 | SD | MET | 1016 | 24.394 | 25.205 | 24.304 | 1.00 | 13.10 |
| ATOM | 1336 | CE | MET | 1016 | 25.503 | 24.227 | 23.277 | 1.00 | 10.89 |
| ATOM | 1337 | C | MET | 1016 | 27.273 | 27.909 | 22.142 | 1.00 | 15.02 |
| ATOM | 1338 | O | MET | 1016 | 28.168 | 27.076 | 21.932 | 1.00 | 13.91 |
| ATOM | 1339 | N | GLU | 1017 | 27.369 | 29.096 | 21.553 | 1.00 | 13.53 |
| ATOM | 1340 | H | GLU | 1017 | 26.602 | 29.699 | 21.620 | 1.00 | 0.00 |
| ATOM | 1341 | CA | GLU | 1017 | 28.499 | 29.493 | 20.729 | 1.00 | 10.68 |
| ATOM | 1342 | CB | GLU | 1017 | 28.217 | 30.909 | 20.136 | 1.00 | 10.74 |
| ATOM | 1343 | CG | GLU | 1017 | 29.468 | 31.336 | 19.402 | 1.00 | 10.82 |
| ATOM | 1344 | CD | GLU | 1017 | 29.247 | 32.722 | 18.829 | 1.00 | 17.03 |
| ATOM | 1345 | OE1 | GLU | 1017 | 28.787 | 33.597 | 19.583 | 1.00 | 19.17 |
| ATOM | 1346 | OE2 | GLU | 1017 | 29.574 | 32.924 | 17.655 | 1.00 | 17.37 |
| ATOM | 1347 | C | GLU | 1017 | 29.705 | 29.529 | 21.613 | 1.00 | 11.76 |
| ATOM | 1348 | O | GLU | 1017 | 30.788 | 29.181 | 21.213 | 1.00 | 15.59 |
| ATOM | 1349 | N | PHE | 1018 | 29.572 | 30.068 | 22.821 | 1.00 | 14.03 |
| ATOM | 1350 | H | PHE | 1018 | 28.724 | 30.503 | 23.046 | 1.00 | 0.00 |
| ATOM | 1351 | CA | PHE | 1018 | 30.653 | 30.112 | 23.784 | 1.00 | 10.71 |
| ATOM | 1352 | CB | PHE | 1018 | 30.179 | 30.801 | 25.013 | 1.00 | 10.03 |
| ATOM | 1353 | CG | PHE | 1018 | 31.278 | 30.803 | 26.015 | 1.00 | 12.80 |
| ATOM | 1354 | CD1 | PHE | 1018 | 31.169 | 30.041 | 27.206 | 1.00 | 13.32 |
| ATOM | 1355 | CD2 | PHE | 1018 | 32.427 | 31.572 | 25.783 | 1.00 | 15.03 |
| ATOM | 1356 | CE1 | PHE | 1018 | 32.233 | 30.063 | 28.166 | 1.00 | 11.64 |
| ATOM | 1357 | CE2 | PHE | 1018 | 33.483 | 31.602 | 26.729 | 1.00 | 15.59 |
| ATOM | 1358 | CZ | PHE | 1018 | 33.363 | 30.833 | 27.924 | 1.00 | 11.76 |
| ATOM | 1359 | C | PHE | 1018 | 31.018 | 28.681 | 24.120 | 1.00 | 14.07 |
| ATOM | 1360 | O | PHE | 1018 | 32.166 | 28.273 | 24.026 | 1.00 | 16.87 |
| ATOM | 1361 | N | LEU | 1019 | 30.067 | 27.816 | 24.522 | 1.00 | 15.90 |
| ATOM | 1362 | H | LEU | 1019 | 29.163 | 28.152 | 24.666 | 1.00 | 0.00 |
| ATOM | 1363 | CA | LEU | 1019 | 30.397 | 26.399 | 24.828 | 1.00 | 15.29 |
| ATOM | 1364 | CB | LEU | 1019 | 29.155 | 25.509 | 25.189 | 1.00 | 16.00 |
| ATOM | 1365 | CG | LEU | 1019 | 28.512 | 25.662 | 26.614 | 1.00 | 18.20 |
| ATOM | 1366 | CD1 | LEU | 1019 | 27.336 | 24.673 | 26.887 | 1.00 | 16.75 |
| ATOM | 1367 | CD2 | LEU | 1019 | 29.623 | 25.428 | 27.598 | 1.00 | 16.35 |
| ATOM | 1368 | C | LEU | 1019 | 31.074 | 25.714 | 23.655 | 1.00 | 15.79 |
| ATOM | 1369 | O | LEU | 1019 | 32.009 | 24.930 | 23.832 | 1.00 | 16.96 |
| ATOM | 1370 | N | ALA | 1020 | 30.592 | 25.947 | 22.448 | 1.00 | 18.47 |
| ATOM | 1371 | H | ALA | 1020 | 29.841 | 26.562 | 22.364 | 1.00 | 0.00 |
| ATOM | 1372 | CA | ALA | 1020 | 31.198 | 25.330 | 21.218 | 1.00 | 19.77 |
| ATOM | 1373 | CB | ALA | 1020 | 30.509 | 25.729 | 19.904 | 1.00 | 19.49 |
| ATOM | 1374 | C | ALA | 1020 | 32.611 | 25.816 | 21.131 | 1.00 | 19.68 |
| ATOM | 1375 | O | ALA | 1020 | 33.511 | 25.024 | 20.854 | 1.00 | 23.80 |
| ATOM | 1376 | N | SER | 1021 | 32.865 | 27.079 | 21.447 | 1.00 | 16.59 |
| ATOM | 1377 | H | SER | 1021 | 32.128 | 27.700 | 21.642 | 1.00 | 0.00 |
| ATOM | 1378 | CA | SER | 1021 | 34.258 | 27.532 | 21.413 | 1.00 | 15.27 |
| ATOM | 1379 | CB | SER | 1021 | 34.296 | 29.069 | 21.570 | 1.00 | 13.71 |
| ATOM | 1380 | OG | SER | 1021 | 34.358 | 29.536 | 22.889 | 1.00 | 13.38 |
| ATOM | 1381 | HG | SER | 1021 | 34.119 | 30.457 | 22.820 | 1.00 | 0.00 |
| ATOM | 1382 | C | SER | 1021 | 35.178 | 26.857 | 22.476 | 1.00 | 17.17 |
| ATOM | 1383 | O | SER | 1021 | 36.414 | 26.835 | 22.313 | 1.00 | 17.74 |
| ATOM | 1384 | N | ARG | 1022 | 34.658 | 26.366 | 23.620 | 1.00 | 13.31 |
| ATOM | 1385 | H | ARG | 1022 | 33.719 | 26.572 | 23.799 | 1.00 | 0.00 |
| ATOM | 1386 | CA | ARG | 1022 | 35.512 | 25.687 | 24.607 | 1.00 | 14.25 |
| ATOM | 1387 | CB | ARG | 1022 | 34.920 | 25.772 | 26.034 | 1.00 | 17.34 |
| ATOM | 1388 | CG | ARG | 1022 | 34.323 | 27.182 | 26.272 | 1.00 | 22.04 |
| ATOM | 1389 | CD | ARG | 1022 | 35.186 | 28.349 | 26.781 | 1.00 | 28.72 |
| ATOM | 1390 | NE | ARG | 1022 | 36.468 | 28.434 | 26.136 | 1.00 | 30.93 |
| ATOM | 1391 | HE | ARG | 1022 | 36.549 | 28.865 | 25.259 | 1.00 | 0.00 |
| ATOM | 1392 | CZ | ARG | 1022 | 37.573 | 27.962 | 26.727 | 1.00 | 35.06 |
| ATOM | 1393 | NH1 | ARG | 1022 | 38.711 | 28.036 | 26.079 | 1.00 | 35.42 |
| ATOM | 1394 | HH11 | ARG | 1022 | 38.728 | 28.452 | 25.170 | 1.00 | 0.00 |
| ATOM | 1395 | HH12 | ARG | 1022 | 39.555 | 27.681 | 26.480 | 1.00 | 0.00 |
| ATOM | 1396 | NH2 | ARG | 1022 | 37.675 | 27.432 | 27.928 | 1.00 | 34.97 |
| ATOM | 1397 | HH21 | ARG | 1022 | 36.879 | 27.331 | 28.522 | 1.00 | 0.00 |
| ATOM | 1398 | HH22 | ARG | 1022 | 38.573 | 27.115 | 28.242 | 1.00 | 0.00 |
| ATOM | 1399 | C | ARG | 1022 | 35.627 | 24.233 | 24.241 | 1.00 | 15.05 |
| ATOM | 1400 | O | ARG | 1022 | 36.083 | 23.383 | 24.991 | 1.00 | 13.70 |
| ATOM | 1401 | N | LYS | 1023 | 35.069 | 23.867 | 23.094 | 1.00 | 17.35 |
| ATOM | 1402 | H | LYS | 1023 | 34.662 | 24.571 | 22.547 | 1.00 | 0.00 |
| ATOM | 1403 | CA | LYS | 1023 | 35.065 | 22.511 | 22.565 | 1.00 | 20.35 |
| ATOM | 1404 | CB | LYS | 1023 | 36.570 | 22.004 | 22.440 | 1.00 | 24.77 |
| ATOM | 1405 | CG | LYS | 1023 | 37.278 | 22.760 | 21.289 | 1.00 | 29.95 |
| ATOM | 1406 | CD | LYS | 1023 | 38.799 | 22.874 | 21.316 | 1.00 | 37.28 |
| ATOM | 1407 | CE | LYS | 1023 | 39.339 | 24.132 | 20.512 | 1.00 | 41.78 |
| ATOM | 1408 | NZ | LYS | 1023 | 39.036 | 24.181 | 19.055 | 1.00 | 41.59 |
| ATOM | 1409 | HZ1 | LYS | 1023 | 38.021 | 24.018 | 18.889 | 1.00 | 0.00 |
| ATOM | 1410 | HZ2 | LYS | 1023 | 39.589 | 23.447 | 18.563 | 1.00 | 0.00 |
| ATOM | 1411 | HZ3 | LYS | 1023 | 39.306 | 25.116 | 18.690 | 1.00 | 0.00 |
| ATOM | 1412 | C | LYS | 1023 | 34.217 | 21.544 | 23.392 | 1.00 | 21.78 |
| ATOM | 1413 | O | LYS | 1023 | 34.426 | 20.341 | 23.462 | 1.00 | 23.61 |
| ATOM | 1414 | N | CYS | 1024 | 33.139 | 22.037 | 23.959 | 1.00 | 20.62 |
| ATOM | 1415 | H | CYS | 1024 | 32.947 | 22.983 | 23.791 | 1.00 | 0.00 |
| ATOM | 1416 | CA | CYS | 1024 | 32.212 | 21.225 | 24.733 | 1.00 | 18.86 |
| ATOM | 1417 | CB | CYS | 1024 | 31.968 | 21.964 | 26.120 | 1.00 | 18.55 |
| ATOM | 1418 | SG | CYS | 1024 | 33.459 | 21.932 | 27.141 | 1.00 | 22.86 |
| ATOM | 1419 | C | CYS | 1024 | 30.890 | 20.994 | 23.953 | 1.00 | 19.40 |
| ATOM | 1420 | O | CYS | 1024 | 30.577 | 21.748 | 23.017 | 1.00 | 18.72 |
| ATOM | 1421 | N | ILE | 1025 | 30.155 | 19.870 | 24.136 | 1.00 | 17.97 |
| ATOM | 1422 | H | ILE | 1025 | 30.490 | 19.168 | 24.730 | 1.00 | 0.00 |
| ATOM | 1423 | CA | ILE | 1025 | 28.837 | 19.762 | 23.485 | 1.00 | 18.59 |
| ATOM | 1424 | CB | ILE | 1025 | 28.686 | 18.597 | 22.365 | 1.00 | 21.68 |
| ATOM | 1425 | CG2 | ILE | 1025 | 29.934 | 18.300 | 21.494 | 1.00 | 20.32 |
| ATOM | 1426 | CG1 | ILE | 1025 | 28.314 | 17.360 | 23.039 | 1.00 | 22.82 |
| ATOM | 1427 | CD1 | ILE | 1025 | 27.012 | 17.051 | 22.294 | 1.00 | 26.88 |
| ATOM | 1428 | C | ILE | 1025 | 27.927 | 19.525 | 24.732 | 1.00 | 18.97 |
| ATOM | 1429 | O | ILE | 1025 | 28.293 | 18.919 | 25.754 | 1.00 | 18.79 |
| ATOM | 1430 | N | HIS | 1026 | 26.801 | 20.226 | 24.817 | 1.00 | 17.43 |
| ATOM | 1431 | H | HIS | 1026 | 26.751 | 21.057 | 24.313 | 1.00 | 0.00 |
| ATOM | 1432 | CA | HIS | 1026 | 25.943 | 20.061 | 26.018 | 1.00 | 16.57 |
| ATOM | 1433 | C | HIS | 1026 | 25.350 | 18.643 | 26.249 | 1.00 | 17.77 |
| ATOM | 1434 | O | HIS | 1026 | 25.476 | 18.075 | 27.354 | 1.00 | 18.77 |
| ATOM | 1435 | CB | HIS | 1026 | 24.817 | 21.191 | 25.915 | 1.00 | 11.94 |
| ATOM | 1436 | CG | HIS | 1026 | 24.100 | 21.347 | 27.220 | 1.00 | 13.66 |
| ATOM | 1437 | ND1 | HIS | 1026 | 23.234 | 20.443 | 27.741 | 1.00 | 11.48 |
| ATOM | 1438 | CE1 | HIS | 1026 | 22.688 | 20.963 | 28.889 | 1.00 | 9.73 |
| ATOM | 1439 | CD2 | HIS | 1026 | 24.085 | 22.445 | 28.090 | 1.00 | 13.13 |
| ATOM | 1440 | NE2 | HIS | 1026 | 23.205 | 22.180 | 29.104 | 1.00 | 14.95 |
| ATOM | 1441 | HE2 | HIS | 1026 | 22.964 | 22.754 | 29.859 | 1.00 | 0.00 |
| ATOM | 1442 | N | ARG | 1027 | 24.772 | 18.064 | 25.150 | 1.00 | 15.57 |
| ATOM | 1443 | H | ARG | 1027 | 24.950 | 18.529 | 24.315 | 1.00 | 0.00 |
| ATOM | 1444 | CA | ARG | 1027 | 24.062 | 16.743 | 25.147 | 1.00 | 18.90 |
| ATOM | 1445 | CB | ARG | 1027 | 24.864 | 15.578 | 25.769 | 1.00 | 19.24 |
| ATOM | 1446 | CG | ARG | 1027 | 26.051 | 15.222 | 24.856 | 1.00 | 23.09 |
| ATOM | 1447 | CD | ARG | 1027 | 26.960 | 14.196 | 25.511 | 1.00 | 20.79 |
| ATOM | 1448 | NE | ARG | 1027 | 28.178 | 14.020 | 24.702 | 1.00 | 24.03 |
| ATOM | 1449 | HE | ARG | 1027 | 28.192 | 13.388 | 23.950 | 1.00 | 0.00 |
| ATOM | 1450 | CZ | ARG | 1027 | 29.260 | 14.742 | 25.014 | 1.00 | 24.19 |
| ATOM | 1451 | NH1 | ARG | 1027 | 30.287 | 14.485 | 24.244 | 1.00 | 25.21 |
| ATOM | 1452 | HH11 | ARG | 1027 | 30.198 | 13.810 | 23.514 | 1.00 | 0.00 |
| ATOM | 1453 | HH12 | ARG | 1027 | 31.150 | 14.970 | 24.381 | 1.00 | 0.00 |
| ATOM | 1454 | NH2 | ARG | 1027 | 29.351 | 15.727 | 25.980 | 1.00 | 20.33 |
| ATOM | 1455 | HH21 | ARG | 1027 | 28.554 | 15.971 | 26.534 | 1.00 | 0.00 |
| ATOM | 1456 | HH22 | ARG | 1027 | 30.215 | 16.210 | 26.125 | 1.00 | 0.00 |
| ATOM | 1457 | C | ARG | 1027 | 22.702 | 16.732 | 25.886 | 1.00 | 20.40 |
| ATOM | 1458 | O | ARG | 1027 | 22.064 | 15.697 | 26.033 | 1.00 | 26.16 |
| ATOM | 1459 | N | ASP | 1028 | 22.275 | 17.820 | 26.527 | 1.00 | 18.68 |
| ATOM | 1460 | H | ASP | 1028 | 22.916 | 18.499 | 26.836 | 1.00 | 0.00 |
| ATOM | 1461 | CA | ASP | 1028 | 20.919 | 17.795 | 27.088 | 1.00 | 18.80 |
| ATOM | 1462 | CB | ASP | 1028 | 20.928 | 17.298 | 28.557 | 1.00 | 19.67 |
| ATOM | 1463 | CG | ASP | 1028 | 19.470 | 16.931 | 29.081 | 1.00 | 16.18 |
| ATOM | 1464 | OD1 | ASP | 1028 | 19.354 | 16.528 | 30.229 | 1.00 | 19.57 |
| ATOM | 1465 | OD2 | ASP | 1028 | 18.455 | 17.030 | 28.408 | 1.00 | 19.03 |
| ATOM | 1466 | C | ASP | 1028 | 20.325 | 19.230 | 27.009 | 1.00 | 17.74 |
| ATOM | 1467 | O | ASP | 1028 | 19.687 | 19.770 | 27.943 | 1.00 | 15.48 |
| ATOM | 1468 | N | LEU | 1029 | 20.526 | 19.899 | 25.856 | 1.00 | 14.51 |
| ATOM | 1469 | H | LEU | 1029 | 20.873 | 19.395 | 25.092 | 1.00 | 0.00 |
| ATOM | 1470 | CA | LEU | 1029 | 19.989 | 21.289 | 25.785 | 1.00 | 13.59 |
| ATOM | 1471 | CB | LEU | 1029 | 20.640 | 21.999 | 24.556 | 1.00 | 12.09 |
| ATOM | 1472 | CG | LEU | 1029 | 20.221 | 23.447 | 24.425 | 1.00 | 10.45 |
| ATOM | 1473 | CD1 | LEU | 1029 | 20.750 | 24.273 | 25.603 | 1.00 | 13.18 |
| ATOM | 1474 | CD2 | LEU | 1029 | 20.777 | 23.972 | 23.154 | 1.00 | 6.48 |
| ATOM | 1475 | C | LEU | 1029 | 18.444 | 21.273 | 25.722 | 1.00 | 12.51 |
| ATOM | 1476 | O | LEU | 1029 | 17.827 | 20.535 | 24.945 | 1.00 | 16.17 |
| ATOM | 1477 | N | ALA | 1030 | 17.776 | 21.982 | 26.597 | 1.00 | 12.00 |
| ATOM | 1478 | H | ALA | 1030 | 18.307 | 22.465 | 27.269 | 1.00 | 0.00 |
| ATOM | 1479 | CA | ALA | 1030 | 16.326 | 22.084 | 26.619 | 1.00 | 10.30 |
| ATOM | 1480 | CB | ALA | 1030 | 15.709 | 20.863 | 27.231 | 1.00 | 8.96 |
| ATOM | 1481 | C | ALA | 1030 | 15.938 | 23.265 | 27.522 | 1.00 | 12.32 |
| ATOM | 1482 | O | ALA | 1030 | 16.714 | 23.679 | 28.371 | 1.00 | 12.27 |
| ATOM | 1483 | N | ALA | 1031 | 14.744 | 23.832 | 27.422 | 1.00 | 13.33 |
| ATOM | 1484 | H | ALA | 1031 | 14.174 | 23.519 | 26.697 | 1.00 | 0.00 |
| ATOM | 1485 | CA | ALA | 1031 | 14.299 | 24.932 | 28.294 | 1.00 | 14.25 |
| ATOM | 1486 | CB | ALA | 1031 | 12.823 | 25.319 | 28.026 | 1.00 | 12.02 |
| ATOM | 1487 | C | ALA | 1031 | 14.432 | 24.585 | 29.793 | 1.00 | 15.72 |
| ATOM | 1488 | O | ALA | 1031 | 14.701 | 25.468 | 30.587 | 1.00 | 16.52 |
| ATOM | 1489 | N | ARG | 1032 | 14.191 | 23.318 | 30.237 | 1.00 | 15.44 |
| ATOM | 1490 | H | ARG | 1032 | 13.834 | 22.721 | 29.556 | 1.00 | 0.00 |
| ATOM | 1491 | CA | ARG | 1032 | 14.328 | 22.906 | 31.651 | 1.00 | 13.01 |
| ATOM | 1492 | CB | ARG | 1032 | 13.880 | 21.454 | 31.815 | 1.00 | 15.20 |
| ATOM | 1493 | CG | ARG | 1032 | 14.814 | 20.471 | 31.101 | 1.00 | 11.06 |
| ATOM | 1494 | CD | ARG | 1032 | 14.708 | 18.945 | 31.355 | 1.00 | 12.37 |
| ATOM | 1495 | NE | ARG | 1032 | 14.354 | 18.612 | 29.991 | 1.00 | 24.51 |
| ATOM | 1496 | HE | ARG | 1032 | 13.451 | 18.952 | 29.802 | 1.00 | 0.00 |
| ATOM | 1497 | CZ | ARG | 1032 | 14.832 | 17.961 | 28.929 | 1.00 | 16.38 |
| ATOM | 1498 | NH1 | ARG | 1032 | 13.952 | 18.110 | 27.993 | 1.00 | 21.10 |
| ATOM | 1499 | HH11 | ARG | 1032 | 13.117 | 18.620 | 28.192 | 1.00 | 0.00 |
| ATOM | 1500 | HH12 | ARG | 1032 | 14.098 | 17.722 | 27.083 | 1.00 | 0.00 |
| ATOM | 1501 | NH2 | ARG | 1032 | 15.872 | 17.231 | 28.651 | 1.00 | 17.09 |
| ATOM | 1502 | HH21 | ARG | 1032 | 16.552 | 17.050 | 29.360 | 1.00 | 0.00 |
| ATOM | 1503 | HH22 | ARG | 1032 | 15.987 | 16.855 | 27.728 | 1.00 | 0.00 |
| ATOM | 1504 | C | ARG | 1032 | 15.782 | 23.053 | 32.099 | 1.00 | 14.65 |
| ATOM | 1505 | O | ARG | 1032 | 16.089 | 23.075 | 33.285 | 1.00 | 14.07 |
| ATOM | 1506 | N | ASN | 1033 | 16.736 | 23.123 | 31.151 | 1.00 | 13.70 |
| ATOM | 1507 | H | ASN | 1033 | 16.471 | 23.088 | 30.215 | 1.00 | 0.00 |
| ATOM | 1508 | CA | ASN | 1033 | 18.145 | 23.294 | 31.508 | 1.00 | 13.03 |
| ATOM | 1509 | CB | ASN | 1033 | 18.997 | 22.279 | 30.752 | 1.00 | 14.47 |
| ATOM | 1510 | CG | ASN | 1033 | 19.042 | 20.980 | 31.542 | 1.00 | 17.02 |
| ATOM | 1511 | OD1 | ASN | 1033 | 18.979 | 20.944 | 32.780 | 1.00 | 13.85 |
| ATOM | 1512 | ND2 | ASN | 1033 | 19.216 | 19.881 | 30.854 | 1.00 | 11.05 |
| ATOM | 1513 | HD21 | ASN | 1033 | 19.317 | 19.876 | 29.884 | 1.00 | 0.00 |
| ATOM | 1514 | HD22 | ASN | 1033 | 19.235 | 19.055 | 31.387 | 1.00 | 0.00 |
| ATOM | 1515 | C | ASN | 1033 | 18.622 | 24.701 | 31.199 | 1.00 | 14.07 |
| ATOM | 1516 | O | ASN | 1033 | 19.785 | 24.958 | 30.847 | 1.00 | 15.20 |
| ATOM | 1517 | N | ILE | 1034 | 17.729 | 25.651 | 31.378 | 1.00 | 13.60 |
| ATOM | 1518 | H | ILE | 1034 | 16.838 | 25.415 | 31.703 | 1.00 | 0.00 |
| ATOM | 1519 | CA | ILE | 1034 | 17.997 | 27.070 | 31.132 | 1.00 | 12.34 |
| ATOM | 1520 | CB | ILE | 1034 | 17.264 | 27.758 | 29.854 | 1.00 | 10.21 |
| ATOM | 1521 | CG2 | ILE | 1034 | 17.838 | 29.252 | 29.745 | 1.00 | 6.13 |
| ATOM | 1522 | CG1 | ILE | 1034 | 17.521 | 27.057 | 28.510 | 1.00 | 5.50 |
| ATOM | 1523 | CD1 | ILE | 1034 | 18.962 | 27.030 | 28.069 | 1.00 | 7.16 |
| ATOM | 1524 | C | ILE | 1034 | 17.431 | 27.770 | 32.332 | 1.00 | 11.73 |
| ATOM | 1525 | O | ILE | 1034 | 16.304 | 27.522 | 32.751 | 1.00 | 14.33 |
| ATOM | 1526 | N | LEU | 1035 | 18.260 | 28.568 | 32.939 | 1.00 | 11.59 |
| ATOM | 1527 | H | LEU | 1035 | 19.178 | 28.575 | 32.616 | 1.00 | 0.00 |
| ATOM | 1528 | CA | LEU | 1035 | 17.874 | 29.379 | 34.105 | 1.00 | 9.84 |
| ATOM | 1529 | CB | LEU | 1035 | 18.972 | 29.275 | 35.195 | 1.00 | 12.34 |
| ATOM | 1530 | CG | LEU | 1035 | 19.183 | 27.762 | 35.640 | 1.00 | 10.77 |
| ATOM | 1531 | CD1 | LEU | 1035 | 20.510 | 27.639 | 36.291 | 1.00 | 8.47 |
| ATOM | 1532 | CD2 | LEU | 1035 | 18.004 | 27.270 | 36.518 | 1.00 | 13.80 |
| ATOM | 1533 | C | LEU | 1035 | 17.603 | 30.852 | 33.813 | 1.00 | 12.07 |
| ATOM | 1534 | O | LEU | 1035 | 18.276 | 31.525 | 33.049 | 1.00 | 10.12 |
| ATOM | 1535 | N | LEU | 1036 | 16.568 | 31.396 | 34.425 | 1.00 | 12.97 |
| ATOM | 1536 | H | LEU | 1036 | 16.015 | 30.823 | 34.983 | 1.00 | 0.00 |
| ATOM | 1537 | CA | LEU | 1036 | 16.271 | 32.809 | 34.282 | 1.00 | 14.34 |
| ATOM | 1538 | CB | LEU | 1036 | 14.721 | 33.069 | 33.980 | 1.00 | 16.55 |
| ATOM | 1539 | CG | LEU | 1036 | 14.380 | 34.573 | 33.889 | 1.00 | 14.26 |
| ATOM | 1540 | CD1 | LEU | 1036 | 15.114 | 35.196 | 32.701 | 1.00 | 10.48 |
| ATOM | 1541 | CD2 | LEU | 1036 | 12.890 | 34.731 | 33.852 | 1.00 | 11.52 |
| ATOM | 1542 | C | LEU | 1036 | 16.682 | 33.512 | 35.591 | 1.00 | 15.87 |
| ATOM | 1543 | O | LEU | 1036 | 16.414 | 33.155 | 36.748 | 1.00 | 11.54 |
| ATOM | 1544 | N | SER | 1037 | 17.546 | 34.456 | 35.344 | 1.00 | 15.63 |
| ATOM | 1545 | H | SER | 1037 | 17.808 | 34.616 | 34.417 | 1.00 | 0.00 |
| ATOM | 1546 | CA | SER | 1037 | 18.039 | 35.288 | 36.411 | 1.00 | 20.83 |
| ATOM | 1547 | CB | SER | 1037 | 19.601 | 35.328 | 36.285 | 1.00 | 20.55 |
| ATOM | 1548 | OG | SER | 1037 | 20.154 | 35.992 | 37.403 | 1.00 | 25.42 |
| ATOM | 1549 | HG | SER | 1037 | 21.123 | 35.913 | 37.333 | 1.00 | 0.00 |
| ATOM | 1550 | C | SER | 1037 | 17.395 | 36.678 | 36.334 | 1.00 | 21.58 |
| ATOM | 1551 | O | SER | 1037 | 16.378 | 36.962 | 35.705 | 1.00 | 19.58 |
| ATOM | 1552 | N | GLU | 1038 | 17.919 | 37.614 | 37.079 | 1.00 | 27.95 |
| ATOM | 1553 | H | GLU | 1038 | 18.679 | 37.369 | 37.649 | 1.00 | 0.00 |
| ATOM | 1554 | CA | GLU | 1038 | 17.390 | 39.000 | 37.052 | 1.00 | 32.87 |
| ATOM | 1555 | CB | GLU | 1038 | 17.911 | 39.855 | 38.195 | 1.00 | 36.53 |
| ATOM | 1556 | CG | GLU | 1038 | 17.118 | 39.444 | 39.435 | 1.00 | 46.24 |
| ATOM | 1557 | CD | GLU | 1038 | 18.121 | 39.462 | 40.562 | 1.00 | 51.35 |
| ATOM | 1558 | OE1 | GLU | 1038 | 19.147 | 38.766 | 40.459 | 1.00 | 55.47 |
| ATOM | 1559 | OE2 | GLU | 1038 | 17.860 | 40.178 | 41.531 | 1.00 | 55.38 |
| ATOM | 1560 | C | GLU | 1038 | 17.816 | 39.723 | 35.796 | 1.00 | 33.53 |
| ATOM | 1561 | O | GLU | 1038 | 18.759 | 39.299 | 35.110 | 1.00 | 35.28 |
| ATOM | 1562 | N | LYS | 1039 | 17.156 | 40.853 | 35.521 | 1.00 | 31.56 |
| ATOM | 1563 | H | LYS | 1039 | 16.510 | 41.245 | 36.115 | 1.00 | 0.00 |
| ATOM | 1564 | CA | LYS | 1039 | 17.466 | 41.650 | 34.375 | 1.00 | 29.59 |
| ATOM | 1565 | CB | LYS | 1039 | 18.827 | 42.349 | 34.702 | 1.00 | 35.25 |
| ATOM | 1566 | CG | LYS | 1039 | 18.760 | 43.381 | 35.882 | 1.00 | 40.79 |
| ATOM | 1567 | CD | LYS | 1039 | 17.831 | 44.649 | 35.672 | 1.00 | 49.90 |
| ATOM | 1568 | CE | LYS | 1039 | 16.237 | 44.548 | 35.460 | 1.00 | 53.40 |
| ATOM | 1569 | NZ | LYS | 1039 | 15.722 | 44.663 | 34.050 | 1.00 | 58.09 |
| ATOM | 1570 | HZ1 | LYS | 1039 | 16.295 | 44.072 | 33.416 | 1.00 | 0.00 |
| ATOM | 1571 | HZ2 | LYS | 1039 | 15.779 | 45.651 | 33.729 | 1.00 | 0.00 |
| ATOM | 1572 | HZ3 | LYS | 1039 | 14.731 | 44.353 | 33.991 | 1.00 | 0.00 |
| ATOM | 1573 | C | LYS | 1039 | 17.449 | 40.881 | 33.091 | 1.00 | 25.93 |
| ATOM | 1574 | O | LYS | 1039 | 18.202 | 41.218 | 32.187 | 1.00 | 28.05 |
| ATOM | 1575 | N | ASN | 1040 | 16.470 | 39.985 | 32.946 | 1.00 | 22.83 |
| ATOM | 1576 | H | ASN | 1040 | 15.799 | 39.899 | 33.658 | 1.00 | 0.00 |
| ATOM | 1577 | CA | ASN | 1040 | 16.247 | 39.110 | 31.781 | 1.00 | 21.34 |
| ATOM | 1578 | CB | ASN | 1040 | 15.637 | 39.920 | 30.584 | 1.00 | 23.65 |
| ATOM | 1579 | CG | ASN | 1040 | 14.261 | 40.168 | 31.110 | 1.00 | 26.52 |
| ATOM | 1580 | OD1 | ASN | 1040 | 13.487 | 39.283 | 31.479 | 1.00 | 27.31 |
| ATOM | 1581 | ND2 | ASN | 1040 | 13.917 | 41.414 | 31.298 | 1.00 | 28.34 |
| ATOM | 1582 | HD21 | ASN | 1040 | 14.520 | 42.154 | 31.109 | 1.00 | 0.00 |
| ATOM | 1583 | HD22 | ASN | 1040 | 13.012 | 41.543 | 31.640 | 1.00 | 0.00 |
| ATOM | 1584 | C | ASN | 1040 | 17.460 | 38.348 | 31.283 | 1.00 | 19.37 |
| ATOM | 1585 | O | ASN | 1040 | 17.549 | 37.980 | 30.085 | 1.00 | 20.36 |
| ATOM | 1586 | N | VAL | 1041 | 18.389 | 38.062 | 32.230 | 1.00 | 17.37 |
| ATOM | 1587 | H | VAL | 1041 | 18.271 | 38.373 | 33.147 | 1.00 | 0.00 |
| ATOM | 1588 | CA | VAL | 1041 | 19.598 | 37.322 | 31.840 | 1.00 | 15.83 |
| ATOM | 1589 | CB | VAL | 1041 | 20.895 | 37.689 | 32.646 | 1.00 | 15.19 |
| ATOM | 1590 | CG1 | VAL | 1041 | 22.045 | 36.725 | 32.224 | 1.00 | 12.64 |
| ATOM | 1591 | CG2 | VAL | 1041 | 21.284 | 39.187 | 32.374 | 1.00 | 13.81 |
| ATOM | 1592 | C | VAL | 1041 | 19.339 | 35.865 | 32.014 | 1.00 | 14.38 |
| ATOM | 1593 | O | VAL | 1041 | 18.931 | 35.413 | 33.064 | 1.00 | 17.24 |
| ATOM | 1594 | N | VAL | 1042 | 19.424 | 35.146 | 30.905 | 1.00 | 15.52 |
| ATOM | 1595 | H | VAL | 1042 | 19.682 | 35.648 | 30.102 | 1.00 | 0.00 |
| ATOM | 1596 | CA | VAL | 1042 | 19.224 | 33.696 | 30.823 | 1.00 | 14.44 |
| ATOM | 1597 | CB | VAL | 1042 | 18.342 | 33.627 | 29.513 | 1.00 | 17.91 |
| ATOM | 1598 | CG1 | VAL | 1042 | 18.805 | 32.663 | 28.452 | 1.00 | 15.23 |
| ATOM | 1599 | CG2 | VAL | 1042 | 16.913 | 33.391 | 30.068 | 1.00 | 16.59 |
| ATOM | 1600 | C | VAL | 1042 | 20.542 | 32.948 | 30.885 | 1.00 | 14.30 |
| ATOM | 1601 | O | VAL | 1042 | 21.510 | 33.257 | 30.198 | 1.00 | 13.71 |
| ATOM | 1602 | N | LYS | 1043 | 20.571 | 31.869 | 31.637 | 1.00 | 11.64 |
| ATOM | 1603 | H | LYS | 1043 | 19.743 | 31.576 | 32.050 | 1.00 | 0.00 |
| ATOM | 1604 | CA | LYS | 1043 | 21.781 | 31.072 | 31.790 | 1.00 | 12.52 |
| ATOM | 1605 | CB | LYS | 1043 | 22.235 | 31.120 | 33.250 | 1.00 | 11.38 |
| ATOM | 1606 | CG | LYS | 1043 | 23.037 | 32.458 | 33.307 | 1.00 | 16.33 |
| ATOM | 1607 | CD | LYS | 1043 | 23.200 | 32.934 | 34.707 | 1.00 | 17.95 |
| ATOM | 1608 | CE | LYS | 1043 | 23.640 | 34.369 | 34.729 | 1.00 | 21.36 |
| ATOM | 1609 | NZ | LYS | 1043 | 25.032 | 34.373 | 34.269 | 1.00 | 21.25 |
| ATOM | 1610 | HZ1 | LYS | 1043 | 25.118 | 33.834 | 33.386 | 1.00 | 0.00 |
| ATOM | 1611 | HZ2 | LYS | 1043 | 25.605 | 33.908 | 35.003 | 1.00 | 0.00 |
| ATOM | 1612 | HZ3 | LYS | 1043 | 25.341 | 35.354 | 34.118 | 1.00 | 0.00 |
| ATOM | 1613 | C | LYS | 1043 | 21.713 | 29.618 | 31.391 | 1.00 | 11.57 |
| ATOM | 1614 | O | LYS | 1043 | 20.862 | 28.959 | 31.951 | 1.00 | 12.48 |
| ATOM | 1615 | N | ILE | 1044 | 22.585 | 29.063 | 30.519 | 1.00 | 13.34 |
| ATOM | 1616 | H | ILE | 1044 | 23.224 | 29.653 | 30.064 | 1.00 | 0.00 |
| ATOM | 1617 | CA | ILE | 1044 | 22.524 | 27.609 | 30.190 | 1.00 | 12.85 |
| ATOM | 1618 | CB | ILE | 1044 | 23.457 | 27.232 | 28.944 | 1.00 | 12.71 |
| ATOM | 1619 | CG2 | ILE | 1044 | 23.379 | 25.724 | 28.640 | 1.00 | 10.07 |
| ATOM | 1620 | CG1 | ILE | 1044 | 22.924 | 27.946 | 27.656 | 1.00 | 11.89 |
| ATOM | 1621 | CD1 | ILE | 1044 | 24.006 | 27.997 | 26.640 | 1.00 | 10.44 |
| ATOM | 1622 | C | ILE | 1044 | 23.047 | 26.879 | 31.444 | 1.00 | 17.82 |
| ATOM | 1623 | O | ILE | 1044 | 23.978 | 27.364 | 32.127 | 1.00 | 17.24 |
| ATOM | 1624 | N | CYS | 1045 | 22.459 | 25.712 | 31.769 | 1.00 | 18.26 |
| ATOM | 1625 | H | CYS | 1045 | 21.695 | 25.404 | 31.230 | 1.00 | 0.00 |
| ATOM | 1626 | CA | CYS | 1045 | 22.888 | 24.905 | 32.917 | 1.00 | 15.34 |
| ATOM | 1627 | CB | CYS | 1045 | 22.120 | 25.322 | 34.175 | 1.00 | 14.89 |
| ATOM | 1628 | SG | CYS | 1045 | 20.447 | 24.713 | 34.292 | 1.00 | 14.01 |
| ATOM | 1629 | C | CYS | 1045 | 22.613 | 23.427 | 32.649 | 1.00 | 14.90 |
| ATOM | 1630 | O | CYS | 1045 | 22.159 | 23.021 | 31.576 | 1.00 | 15.25 |
| ATOM | 1631 | N | ASP | 1046 | 22.994 | 22.546 | 33.517 | 1.00 | 13.28 |
| ATOM | 1632 | H | ASP | 1046 | 23.441 | 22.858 | 34.319 | 1.00 | 0.00 |
| ATOM | 1633 | CA | ASP | 1046 | 22.676 | 21.144 | 33.333 | 1.00 | 13.21 |
| ATOM | 1634 | CB | ASP | 1046 | 23.881 | 20.456 | 32.606 | 1.00 | 13.57 |
| ATOM | 1635 | CG | ASP | 1046 | 23.500 | 19.000 | 32.353 | 1.00 | 12.65 |
| ATOM | 1636 | OD1 | ASP | 1046 | 24.178 | 18.208 | 32.939 | 1.00 | 21.25 |
| ATOM | 1637 | OD2 | ASP | 1046 | 22.589 | 18.621 | 31.651 | 1.00 | 14.14 |
| ATOM | 1638 | C | ASP | 1046 | 22.352 | 20.547 | 34.722 | 1.00 | 14.27 |
| ATOM | 1639 | O | ASP | 1046 | 23.232 | 20.415 | 35.568 | 1.00 | 14.82 |
| ATOM | 1640 | N | PHE | 1047 | 21.068 | 20.338 | 35.078 | 1.00 | 14.62 |
| ATOM | 1641 | H | PHE | 1047 | 20.347 | 20.639 | 34.483 | 1.00 | 0.00 |
| ATOM | 1642 | CA | PHE | 1047 | 20.770 | 19.762 | 36.387 | 1.00 | 15.32 |
| ATOM | 1643 | CB | PHE | 1047 | 19.270 | 19.757 | 36.672 | 1.00 | 15.41 |
| ATOM | 1644 | CG | PHE | 1047 | 18.779 | 21.141 | 37.080 | 1.00 | 22.06 |
| ATOM | 1645 | CD1 | PHE | 1047 | 18.474 | 22.147 | 36.124 | 1.00 | 20.80 |
| ATOM | 1646 | CD2 | PHE | 1047 | 18.564 | 21.455 | 38.440 | 1.00 | 20.67 |
| ATOM | 1647 | CE1 | PHE | 1047 | 17.969 | 23.410 | 36.512 | 1.00 | 19.44 |
| ATOM | 1648 | CE2 | PHE | 1047 | 18.060 | 22.707 | 38.831 | 1.00 | 20.50 |
| ATOM | 1649 | CZ | PHE | 1047 | 17.759 | 23.691 | 37.875 | 1.00 | 20.35 |
| ATOM | 1650 | C | PHE | 1047 | 21.317 | 18.351 | 36.504 | 1.00 | 14.42 |
| ATOM | 1651 | O | PHE | 1047 | 21.740 | 17.912 | 37.551 | 1.00 | 16.83 |
| ATOM | 1652 | N | GLY | 1048 | 21.374 | 17.569 | 35.439 | 1.00 | 17.98 |
| ATOM | 1653 | H | GLY | 1048 | 20.857 | 17.855 | 34.664 | 1.00 | 0.00 |
| ATOM | 1654 | CA | GLY | 1048 | 21.922 | 16.183 | 35.464 | 1.00 | 17.50 |
| ATOM | 1655 | C | GLY | 1048 | 21.366 | 15.248 | 36.584 | 1.00 | 19.10 |
| ATOM | 1656 | O | GLY | 1048 | 20.152 | 14.991 | 36.649 | 1.00 | 18.83 |
| ATOM | 1657 | N | LEU | 1049 | 22.305 | 14.797 | 37.473 | 1.00 | 17.99 |
| ATOM | 1658 | H | LEU | 1049 | 23.175 | 15.193 | 37.283 | 1.00 | 0.00 |
| ATOM | 1659 | CA | LEU | 1049 | 22.090 | 13.941 | 38.649 | 1.00 | 18.83 |
| ATOM | 1660 | CB | LEU | 1049 | 23.365 | 13.708 | 39.478 | 1.00 | 21.93 |
| ATOM | 1661 | CG | LEU | 1049 | 24.249 | 12.460 | 39.231 | 1.00 | 25.95 |
| ATOM | 1662 | CD1 | LEU | 1049 | 23.930 | 11.765 | 37.868 | 1.00 | 25.79 |
| ATOM | 1663 | CD2 | LEU | 1049 | 25.710 | 12.931 | 39.438 | 1.00 | 26.28 |
| ATOM | 1664 | C | LEU | 1049 | 21.084 | 14.580 | 39.590 | 1.00 | 20.48 |
| ATOM | 1665 | O | LEU | 1049 | 20.380 | 13.867 | 40.306 | 1.00 | 20.14 |
| ATOM | 1666 | N | ALA | 1050 | 20.999 | 15.935 | 39.633 | 1.00 | 19.90 |
| ATOM | 1667 | H | ALA | 1050 | 21.630 | 16.454 | 39.090 | 1.00 | 0.00 |
| ATOM | 1668 | CA | ALA | 1050 | 19.977 | 16.669 | 40.476 | 1.00 | 18.14 |
| ATOM | 1669 | CB | ALA | 1050 | 20.285 | 18.141 | 40.749 | 1.00 | 15.37 |
| ATOM | 1670 | C | ALA | 1050 | 18.613 | 16.743 | 39.831 | 1.00 | 17.52 |
| ATOM | 1671 | O | ALA | 1050 | 17.853 | 17.684 | 40.067 | 1.00 | 17.19 |
| ATOM | 1672 | N | ARG | 1051 | 18.383 | 15.917 | 38.804 | 1.00 | 16.27 |
| ATOM | 1673 | H | ARG | 1051 | 19.103 | 15.344 | 38.468 | 1.00 | 0.00 |
| ATOM | 1674 | CA | ARG | 1051 | 17.045 | 15.853 | 38.195 | 1.00 | 17.29 |
| ATOM | 1675 | CB | ARG | 1051 | 17.003 | 16.236 | 36.751 | 1.00 | 15.99 |
| ATOM | 1676 | CG | ARG | 1051 | 15.566 | 16.173 | 36.313 | 1.00 | 14.92 |
| ATOM | 1677 | CD | ARG | 1051 | 15.530 | 16.522 | 34.843 | 1.00 | 16.02 |
| ATOM | 1678 | NE | ARG | 1051 | 15.686 | 17.982 | 34.688 | 1.00 | 18.74 |
| ATOM | 1679 | HE | ARG | 1051 | 14.962 | 18.527 | 35.064 | 1.00 | 0.00 |
| ATOM | 1680 | CZ | ARG | 1051 | 16.703 | 18.673 | 34.115 | 1.00 | 19.42 |
| ATOM | 1681 | NH1 | ARG | 1051 | 16.609 | 19.987 | 34.079 | 1.00 | 19.30 |
| ATOM | 1682 | HH11 | ARG | 1051 | 15.776 | 20.359 | 34.462 | 1.00 | 0.00 |
| ATOM | 1683 | HH12 | ARG | 1051 | 17.307 | 20.607 | 33.702 | 1.00 | 0.00 |
| ATOM | 1684 | NH2 | ARG | 1051 | 17.830 | 18.189 | 33.634 | 1.00 | 20.36 |
| ATOM | 1685 | HH21 | ARG | 1051 | 17.984 | 17.209 | 33.660 | 1.00 | 0.00 |
| ATOM | 1686 | HH22 | ARG | 1051 | 18.505 | 18.814 | 33.237 | 1.00 | 0.00 |
| ATOM | 1687 | C | ARG | 1051 | 16.591 | 14.388 | 38.236 | 1.00 | 18.81 |
| ATOM | 1688 | O | ARG | 1051 | 17.357 | 13.460 | 37.915 | 1.00 | 17.50 |
| ATOM | 1689 | N | ASP | 1052 | 15.387 | 14.162 | 38.759 | 1.00 | 19.47 |
| ATOM | 1690 | H | ASP | 1052 | 14.967 | 14.890 | 39.268 | 1.00 | 0.00 |
| ATOM | 1691 | CA | ASP | 1052 | 14.872 | 12.784 | 38.785 | 1.00 | 23.91 |
| ATOM | 1692 | CB | ASP | 1052 | 13.684 | 12.741 | 39.740 | 1.00 | 27.71 |
| ATOM | 1693 | CG | ASP | 1052 | 13.482 | 11.321 | 40.262 | 1.00 | 34.58 |
| ATOM | 1694 | OD1 | ASP | 1052 | 13.693 | 10.348 | 39.506 | 1.00 | 36.41 |
| ATOM | 1695 | OD2 | ASP | 1052 | 13.118 | 11.202 | 41.445 | 1.00 | 38.78 |
| ATOM | 1696 | C | ASP | 1052 | 14.457 | 12.293 | 37.351 | 1.00 | 24.05 |
| ATOM | 1697 | O | ASP | 1052 | 13.262 | 12.194 | 36.995 | 1.00 | 21.30 |
| ATOM | 1698 | N | ILE | 1053 | 15.465 | 11.901 | 36.549 | 1.00 | 22.81 |
| ATOM | 1699 | H | ILE | 1053 | 16.394 | 11.999 | 36.852 | 1.00 | 0.00 |
| ATOM | 1700 | CA | ILE | 1053 | 15.093 | 11.522 | 35.179 | 1.00 | 25.08 |
| ATOM | 1701 | CB | ILE | 1053 | 16.343 | 11.306 | 34.335 | 1.00 | 24.23 |
| ATOM | 1702 | CG2 | ILE | 1053 | 16.995 | 12.732 | 34.151 | 1.00 | 17.05 |
| ATOM | 1703 | CG1 | ILE | 1053 | 17.171 | 10.120 | 34.896 | 1.00 | 22.09 |
| ATOM | 1704 | CD1 | ILE | 1053 | 18.107 | 9.522 | 33.799 | 1.00 | 16.85 |
| ATOM | 1705 | C | ILE | 1053 | 14.147 | 10.328 | 34.974 | 1.00 | 28.98 |
| ATOM | 1706 | O | ILE | 1053 | 13.601 | 10.168 | 33.879 | 1.00 | 29.80 |
| ATOM | 1707 | N | TYR | 1054 | 13.993 | 9.519 | 36.046 | 1.00 | 29.02 |
| ATOM | 1708 | H | TYR | 1054 | 14.595 | 9.739 | 36.779 | 1.00 | 0.00 |
| ATOM | 1709 | CA | TYR | 1054 | 13.088 | 8.325 | 36.144 | 1.00 | 29.15 |
| ATOM | 1710 | CB | TYR | 1054 | 13.394 | 7.444 | 37.353 | 1.00 | 32.32 |
| ATOM | 1711 | C | TYR | 1054 | 11.651 | 8.707 | 36.313 | 1.00 | 26.81 |
| ATOM | 1712 | O | TYR | 1054 | 10.780 | 8.032 | 35.839 | 1.00 | 29.22 |
| ATOM | 1713 | N | LYS | 1055 | 11.377 | 9.704 | 37.125 | 1.00 | 28.26 |
| ATOM | 1714 | H | LYS | 1055 | 12.148 | 10.165 | 37.503 | 1.00 | 0.00 |
| ATOM | 1715 | CA | LYS | 1055 | 10.007 | 10.218 | 37.333 | 1.00 | 29.35 |
| ATOM | 1716 | CB | LYS | 1055 | 9.924 | 10.859 | 38.749 | 1.00 | 26.02 |
| ATOM | 1717 | C | LYS | 1055 | 9.666 | 11.296 | 36.207 | 1.00 | 30.26 |
| ATOM | 1718 | O | LYS | 1055 | 8.532 | 11.567 | 35.793 | 1.00 | 29.48 |
| ATOM | 1719 | N | ASP | 1056 | 10.707 | 11.994 | 35.702 | 1.00 | 30.51 |
| ATOM | 1720 | H | ASP | 1056 | 11.605 | 11.744 | 35.989 | 1.00 | 0.00 |
| ATOM | 1721 | CA | ASP | 1056 | 10.546 | 13.025 | 34.645 | 1.00 | 26.47 |
| ATOM | 1722 | CB | ASP | 1056 | 11.895 | 13.743 | 34.460 | 1.00 | 25.58 |
| ATOM | 1723 | CG | ASP | 1056 | 11.776 | 15.075 | 33.775 | 1.00 | 24.81 |
| ATOM | 1724 | OD1 | ASP | 1056 | 12.624 | 15.897 | 33.990 | 1.00 | 30.77 |
| ATOM | 1725 | OD2 | ASP | 1056 | 10.888 | 15.317 | 32.993 | 1.00 | 25.77 |
| ATOM | 1726 | C | ASP | 1056 | 10.035 | 12.488 | 33.297 | 1.00 | 25.44 |
| ATOM | 1727 | O | ASP | 1056 | 10.686 | 11.740 | 32.572 | 1.00 | 27.30 |
| ATOM | 1728 | N | PRO | 1057 | 8.882 | 13.000 | 32.838 | 1.00 | 23.99 |
| ATOM | 1729 | CD | PRO | 1057 | 8.106 | 14.128 | 33.408 | 1.00 | 20.38 |
| ATOM | 1730 | CA | PRO | 1057 | 8.298 | 12.468 | 31.631 | 1.00 | 22.12 |
| ATOM | 1731 | CB | PRO | 1057 | 6.933 | 13.051 | 31.638 | 1.00 | 21.61 |
| ATOM | 1732 | CG | PRO | 1057 | 7.056 | 14.387 | 32.377 | 1.00 | 21.32 |
| ATOM | 1733 | C | PRO | 1057 | 9.119 | 12.789 | 30.396 | 1.00 | 24.82 |
| ATOM | 1734 | O | PRO | 1057 | 8.954 | 12.145 | 29.357 | 1.00 | 24.42 |
| ATOM | 1735 | N | ASP | 1058 | 9.986 | 13.853 | 30.473 | 1.00 | 23.82 |
| ATOM | 1736 | H | ASP | 1058 | 9.978 | 14.344 | 31.313 | 1.00 | 0.00 |
| ATOM | 1737 | CA | ASP | 1058 | 10.853 | 14.282 | 29.314 | 1.00 | 21.28 |
| ATOM | 1738 | CB | ASP | 1058 | 11.528 | 15.642 | 29.522 | 1.00 | 19.75 |
| ATOM | 1739 | CG | ASP | 1058 | 10.583 | 16.855 | 29.722 | 1.00 | 22.55 |
| ATOM | 1740 | OD1 | ASP | 1058 | 11.072 | 17.951 | 30.045 | 1.00 | 22.56 |
| ATOM | 1741 | OD2 | ASP | 1058 | 9.341 | 16.734 | 29.548 | 1.00 | 23.31 |
| ATOM | 1742 | C | ASP | 1058 | 11.963 | 13.326 | 28.980 | 1.00 | 22.67 |
| ATOM | 1743 | O | ASP | 1058 | 12.569 | 13.505 | 27.933 | 1.00 | 24.16 |
| ATOM | 1744 | N | TYR | 1059 | 12.300 | 12.363 | 29.860 | 1.00 | 21.34 |
| ATOM | 1745 | H | TYR | 1059 | 11.751 | 12.217 | 30.665 | 1.00 | 0.00 |
| ATOM | 1746 | CA | TYR | 1059 | 13.356 | 11.371 | 29.601 | 1.00 | 20.81 |
| ATOM | 1747 | CB | TYR | 1059 | 14.232 | 11.265 | 30.827 | 1.00 | 19.11 |
| ATOM | 1748 | CG | TYR | 1059 | 14.879 | 12.553 | 30.987 | 1.00 | 15.89 |
| ATOM | 1749 | CD1 | TYR | 1059 | 14.279 | 13.617 | 31.655 | 1.00 | 19.28 |
| ATOM | 1750 | CE1 | TYR | 1059 | 14.964 | 14.850 | 31.725 | 1.00 | 19.64 |
| ATOM | 1751 | CD2 | TYR | 1059 | 16.143 | 12.720 | 30.422 | 1.00 | 19.35 |
| ATOM | 1752 | CE2 | TYR | 1059 | 16.836 | 13.930 | 30.483 | 1.00 | 18.27 |
| ATOM | 1753 | CZ | TYR | 1059 | 16.232 | 15.007 | 31.147 | 1.00 | 20.60 |
| ATOM | 1754 | OH | TYR | 1059 | 16.840 | 16.256 | 31.140 | 1.00 | 22.95 |
| ATOM | 1755 | HH | TYR | 1059 | 16.355 | 16.843 | 31.728 | 1.00 | 0.00 |
| ATOM | 1756 | C | TYR | 1059 | 12.610 | 10.049 | 29.336 | 1.00 | 24.44 |
| ATOM | 1757 | O | TYR | 1059 | 11.732 | 9.643 | 30.099 | 1.00 | 26.74 |
| ATOM | 1758 | N | VAL | 1060 | 12.890 | 9.339 | 28.258 | 1.00 | 23.88 |
| ATOM | 1759 | H | VAL | 1060 | 13.691 | 9.571 | 27.791 | 1.00 | 0.00 |
| ATOM | 1760 | CA | VAL | 1060 | 12.194 | 8.100 | 27.834 | 1.00 | 24.97 |
| ATOM | 1761 | CB | VAL | 1060 | 11.683 | 8.274 | 26.392 | 1.00 | 21.42 |
| ATOM | 1762 | CG1 | VAL | 1060 | 10.984 | 7.050 | 25.851 | 1.00 | 25.63 |
| ATOM | 1763 | CG2 | VAL | 1060 | 10.622 | 9.355 | 26.389 | 1.00 | 17.94 |
| ATOM | 1764 | C | VAL | 1060 | 13.112 | 6.939 | 27.887 | 1.00 | 25.85 |
| ATOM | 1765 | O | VAL | 1060 | 14.279 | 7.059 | 27.539 | 1.00 | 24.87 |
| ATOM | 1766 | N | ARG | 1061 | 12.618 | 5.812 | 28.359 | 1.00 | 28.58 |
| ATOM | 1767 | H | ARG | 1061 | 11.680 | 5.790 | 28.635 | 1.00 | 0.00 |
| ATOM | 1768 | CA | ARG | 1061 | 13.496 | 4.629 | 28.428 | 1.00 | 31.87 |
| ATOM | 1769 | CB | ARG | 1061 | 12.762 | 3.462 | 29.198 | 1.00 | 33.33 |
| ATOM | 1770 | C | ARG | 1061 | 13.919 | 4.138 | 27.006 | 1.00 | 33.41 |
| ATOM | 1771 | O | ARG | 1061 | 13.093 | 4.015 | 26.089 | 1.00 | 34.46 |
| ATOM | 1772 | N | LYS | 1062 | 15.196 | 3.822 | 26.822 | 1.00 | 33.74 |
| ATOM | 1773 | H | LYS | 1062 | 15.787 | 3.955 | 27.592 | 1.00 | 0.00 |
| ATOM | 1774 | CA | LYS | 1062 | 15.769 | 3.343 | 25.566 | 1.00 | 32.88 |
| ATOM | 1775 | CB | LYS | 1062 | 16.147 | 4.477 | 24.610 | 1.00 | 29.93 |
| ATOM | 1776 | C | LYS | 1062 | 17.046 | 2.617 | 25.969 | 1.00 | 36.22 |
| ATOM | 1777 | O | LYS | 1062 | 18.018 | 3.157 | 26.529 | 1.00 | 38.22 |
| ATOM | 1778 | N | GLY | 1063 | 17.057 | 1.303 | 25.740 | 1.00 | 37.33 |
| ATOM | 1779 | H | GLY | 1063 | 16.238 | 0.889 | 25.421 | 1.00 | 0.00 |
| ATOM | 1780 | CA | GLY | 1063 | 18.226 | 0.543 | 26.134 | 1.00 | 36.63 |
| ATOM | 1781 | C | GLY | 1063 | 18.219 | 0.688 | 27.616 | 1.00 | 36.78 |
| ATOM | 1782 | O | GLY | 1063 | 17.162 | 0.794 | 28.226 | 1.00 | 35.79 |
| ATOM | 1783 | N | ASP | 1064 | 19.358 | 0.667 | 28.244 | 1.00 | 39.92 |
| ATOM | 1784 | H | ASP | 1064 | 20.191 | 0.703 | 27.743 | 1.00 | 0.00 |
| ATOM | 1785 | CA | ASP | 1064 | 19.336 | 0.828 | 29.707 | 1.00 | 43.73 |
| ATOM | 1786 | CB | ASP | 1064 | 20.439 | −0.084 | 30.383 | 1.00 | 49.93 |
| ATOM | 1787 | CG | ASP | 1064 | 20.021 | −1.579 | 30.435 | 1.00 | 56.62 |
| ATOM | 1788 | OD1 | ASP | 1064 | 18.810 | −1.894 | 30.520 | 1.00 | 58.79 |
| ATOM | 1789 | OD2 | ASP | 1064 | 20.938 | −2.422 | 30.424 | 1.00 | 62.24 |
| ATOM | 1790 | C | ASP | 1064 | 19.543 | 2.290 | 30.074 | 1.00 | 41.33 |
| ATOM | 1791 | O | ASP | 1064 | 20.230 | 2.623 | 31.032 | 1.00 | 42.96 |
| ATOM | 1792 | N | ALA | 1065 | 18.931 | 3.184 | 29.326 | 1.00 | 37.38 |
| ATOM | 1793 | H | ALA | 1065 | 18.385 | 2.892 | 28.559 | 1.00 | 0.00 |
| ATOM | 1794 | CA | ALA | 1065 | 19.062 | 4.614 | 29.573 | 1.00 | 34.41 |
| ATOM | 1795 | CB | ALA | 1065 | 20.003 | 5.176 | 28.533 | 1.00 | 34.21 |
| ATOM | 1796 | C | ALA | 1065 | 17.739 | 5.372 | 29.481 | 1.00 | 34.30 |
| ATOM | 1797 | O | ALA | 1065 | 16.788 | 4.920 | 28.842 | 1.00 | 34.83 |
| ATOM | 1798 | N | ARG | 1066 | 17.600 | 6.456 | 30.234 | 1.00 | 32.03 |
| ATOM | 1799 | H | ARG | 1066 | 18.342 | 6.663 | 30.843 | 1.00 | 0.00 |
| ATOM | 1800 | CA | ARG | 1066 | 16.406 | 7.336 | 30.160 | 1.00 | 32.47 |
| ATOM | 1801 | CB | ARG | 1066 | 15.903 | 7.681 | 31.547 | 1.00 | 36.10 |
| ATOM | 1802 | CG | ARG | 1066 | 14.694 | 6.784 | 31.643 | 1.00 | 41.06 |
| ATOM | 1803 | CD | ARG | 1066 | 14.197 | 6.571 | 33.080 | 1.00 | 46.90 |
| ATOM | 1804 | NE | ARG | 1066 | 12.826 | 6.154 | 32.855 | 1.00 | 51.11 |
| ATOM | 1805 | HE | ARG | 1066 | 12.600 | 5.200 | 32.900 | 1.00 | 0.00 |
| ATOM | 1806 | CZ | ARG | 1066 | 11.869 | 7.056 | 32.555 | 1.00 | 54.52 |
| ATOM | 1807 | NH1 | ARG | 1066 | 12.017 | 8.381 | 32.470 | 1.00 | 53.44 |
| ATOM | 1808 | HH11 | ARG | 1066 | 12.906 | 8.810 | 32.629 | 1.00 | 0.00 |
| ATOM | 1809 | HH12 | ARG | 1066 | 11.219 | 8.938 | 32.242 | 1.00 | 0.00 |
| ATOM | 1810 | NH2 | ARG | 1066 | 10.712 | 6.577 | 32.116 | 1.00 | 59.55 |
| ATOM | 1811 | HH21 | ARG | 1066 | 10.603 | 5.587 | 32.035 | 1.00 | 0.00 |
| ATOM | 1812 | HH22 | ARG | 1066 | 9.957 | 7.185 | 31.877 | 1.00 | 0.00 |
| ATOM | 1813 | C | ARG | 1066 | 16.858 | 8.608 | 29.404 | 1.00 | 28.88 |
| ATOM | 1814 | O | ARG | 1066 | 17.658 | 9.417 | 29.894 | 1.00 | 24.93 |
| ATOM | 1815 | N | LEU | 1067 | 16.417 | 8.712 | 28.158 | 1.00 | 25.36 |
| ATOM | 1816 | H | LEU | 1067 | 15.873 | 7.971 | 27.817 | 1.00 | 0.00 |
| ATOM | 1817 | CA | LEU | 1067 | 16.835 | 9.800 | 27.288 | 1.00 | 21.52 |
| ATOM | 1818 | CB | LEU | 1067 | 17.323 | 9.121 | 25.979 | 1.00 | 21.29 |
| ATOM | 1819 | CG | LEU | 1067 | 18.435 | 8.033 | 26.086 | 1.00 | 22.20 |
| ATOM | 1820 | CD1 | LEU | 1067 | 18.746 | 7.480 | 24.675 | 1.00 | 25.57 |
| ATOM | 1821 | CD2 | LEU | 1067 | 19.718 | 8.576 | 26.702 | 1.00 | 22.27 |
| ATOM | 1822 | C | LEU | 1067 | 15.861 | 10.936 | 26.981 | 1.00 | 19.98 |
| ATOM | 1823 | O | LEU | 1067 | 14.663 | 10.744 | 26.937 | 1.00 | 19.57 |
| ATOM | 1824 | N | PRO | 1068 | 16.332 | 12.173 | 26.707 | 1.00 | 18.72 |
| ATOM | 1825 | CD | PRO | 1068 | 17.736 | 12.634 | 26.876 | 1.00 | 13.37 |
| ATOM | 1826 | CA | PRO | 1068 | 15.440 | 13.315 | 26.325 | 1.00 | 18.49 |
| ATOM | 1827 | CB | PRO | 1068 | 16.237 | 14.582 | 26.690 | 1.00 | 14.24 |
| ATOM | 1828 | CG | PRO | 1068 | 17.641 | 14.091 | 26.399 | 1.00 | 17.34 |
| ATOM | 1829 | C | PRO | 1068 | 15.140 | 13.174 | 24.845 | 1.00 | 18.62 |
| ATOM | 1830 | O | PRO | 1068 | 15.586 | 13.947 | 23.982 | 1.00 | 18.39 |
| ATOM | 1831 | N | LEU | 1069 | 14.364 | 12.169 | 24.546 | 1.00 | 19.97 |
| ATOM | 1832 | H | LEU | 1069 | 13.958 | 11.653 | 25.277 | 1.00 | 0.00 |
| ATOM | 1833 | CA | LEU | 1069 | 14.056 | 11.845 | 23.131 | 1.00 | 21.38 |
| ATOM | 1834 | CB | LEU | 1069 | 13.172 | 10.559 | 23.058 | 1.00 | 25.64 |
| ATOM | 1835 | CG | LEU | 1069 | 13.887 | 9.156 | 22.738 | 1.00 | 30.42 |
| ATOM | 1836 | CD1 | LEU | 1069 | 15.384 | 9.295 | 22.362 | 1.00 | 32.29 |
| ATOM | 1837 | CD2 | LEU | 1069 | 14.092 | 8.329 | 23.992 | 1.00 | 34.04 |
| ATOM | 1838 | C | LEU | 1069 | 13.407 | 12.940 | 22.331 | 1.00 | 19.16 |
| ATOM | 1839 | O | LEU | 1069 | 13.762 | 13.132 | 21.180 | 1.00 | 18.27 |
| ATOM | 1840 | N | LYS | 1070 | 12.503 | 13.743 | 22.874 | 1.00 | 16.68 |
| ATOM | 1841 | H | LYS | 1070 | 12.243 | 13.548 | 23.791 | 1.00 | 0.00 |
| ATOM | 1842 | CA | LYS | 1070 | 11.899 | 14.837 | 22.084 | 1.00 | 13.98 |
| ATOM | 1843 | CB | LYS | 1070 | 10.787 | 15.458 | 22.884 | 1.00 | 12.88 |
| ATOM | 1844 | CG | LYS | 1070 | 9.681 | 14.478 | 23.006 | 1.00 | 13.74 |
| ATOM | 1845 | CD | LYS | 1070 | 8.381 | 15.112 | 23.434 | 1.00 | 14.34 |
| ATOM | 1846 | CE | LYS | 1070 | 8.463 | 15.402 | 24.900 | 1.00 | 17.96 |
| ATOM | 1847 | NZ | LYS | 1070 | 7.099 | 15.808 | 25.175 | 1.00 | 22.51 |
| ATOM | 1848 | HZ1 | LYS | 1070 | 6.455 | 15.075 | 24.817 | 1.00 | 0.00 |
| ATOM | 1849 | HZ2 | LYS | 1070 | 6.900 | 16.703 | 24.684 | 1.00 | 0.00 |
| ATOM | 1850 | HZ3 | LYS | 1070 | 6.952 | 15.939 | 26.194 | 1.00 | 0.00 |
| ATOM | 1851 | C | LYS | 1070 | 12.865 | 15.966 | 21.639 | 1.00 | 14.66 |
| ATOM | 1852 | O | LYS | 1070 | 12.552 | 16.846 | 20.813 | 1.00 | 13.66 |
| ATOM | 1853 | N | TRP | 1071 | 14.008 | 16.024 | 22.353 | 1.00 | 15.80 |
| ATOM | 1854 | H | TRP | 1071 | 14.182 | 15.338 | 23.024 | 1.00 | 0.00 |
| ATOM | 1855 | CA | TRP | 1071 | 15.049 | 17.029 | 22.078 | 1.00 | 15.62 |
| ATOM | 1856 | CB | TRP | 1071 | 15.577 | 17.594 | 23.435 | 1.00 | 11.52 |
| ATOM | 1857 | CG | TRP | 1071 | 14.591 | 18.392 | 24.204 | 1.00 | 11.83 |
| ATOM | 1858 | CD2 | TRP | 1071 | 13.434 | 18.010 | 24.968 | 1.00 | 11.84 |
| ATOM | 1859 | CE2 | TRP | 1071 | 12.742 | 19.186 | 25.317 | 1.00 | 14.93 |
| ATOM | 1860 | CE3 | TRP | 1071 | 12.904 | 16.821 | 25.403 | 1.00 | 10.67 |
| ATOM | 1861 | CD1 | TRP | 1071 | 14.526 | 19.783 | 24.153 | 1.00 | 14.72 |
| ATOM | 1862 | NE1 | TRP | 1071 | 13.415 | 20.256 | 24.815 | 1.00 | 14.26 |
| ATOM | 1863 | HE1 | TRP | 1071 | 13.025 | 21.141 | 24.721 | 1.00 | 0.00 |
| ATOM | 1864 | CZ2 | TRP | 1071 | 11.577 | 19.115 | 26.065 | 1.00 | 12.02 |
| ATOM | 1865 | CZ3 | TRP | 1071 | 11.749 | 16.742 | 26.133 | 1.00 | 10.87 |
| ATOM | 1866 | CH2 | TRP | 1071 | 11.091 | 17.887 | 26.454 | 1.00 | 11.25 |
| ATOM | 1867 | C | TRP | 1071 | 16.195 | 16.451 | 21.247 | 1.00 | 17.67 |
| ATOM | 1868 | O | TRP | 1071 | 17.083 | 17.183 | 20.795 | 1.00 | 20.11 |
| ATOM | 1869 | N | MET | 1072 | 16.168 | 15.140 | 21.004 | 1.00 | 15.36 |
| ATOM | 1870 | H | MET | 1072 | 15.360 | 14.629 | 21.219 | 1.00 | 0.00 |
| ATOM | 1871 | CA | MET | 1072 | 17.211 | 14.535 | 20.244 | 1.00 | 16.94 |
| ATOM | 1872 | CB | MET | 1072 | 17.416 | 13.125 | 20.798 | 1.00 | 17.87 |
| ATOM | 1873 | CG | MET | 1072 | 18.138 | 13.046 | 22.138 | 1.00 | 18.88 |
| ATOM | 1874 | SD | MET | 1072 | 17.770 | 11.425 | 22.918 | 1.00 | 22.37 |
| ATOM | 1875 | CE | MET | 1072 | 19.300 | 10.549 | 22.374 | 1.00 | 11.18 |
| ATOM | 1876 | C | MET | 1072 | 17.126 | 14.489 | 18.735 | 1.00 | 16.60 |
| ATOM | 1877 | O | MET | 1072 | 16.131 | 14.234 | 18.061 | 1.00 | 20.21 |
| ATOM | 1878 | N | ALA | 1073 | 18.290 | 14.793 | 18.181 | 1.00 | 17.70 |
| ATOM | 1879 | H | ALA | 1073 | 18.936 | 15.239 | 18.768 | 1.00 | 0.00 |
| ATOM | 1880 | CA | ALA | 1073 | 18.508 | 14.752 | 16.725 | 1.00 | 19.07 |
| ATOM | 1881 | CB | ALA | 1073 | 19.918 | 15.368 | 16.423 | 1.00 | 14.82 |
| ATOM | 1882 | C | ALA | 1073 | 18.403 | 13.335 | 16.181 | 1.00 | 18.68 |
| ATOM | 1883 | O | ALA | 1073 | 18.850 | 12.373 | 16.811 | 1.00 | 18.85 |
| ATOM | 1884 | N | PRO | 1074 | 17.958 | 13.118 | 14.972 | 1.00 | 21.84 |
| ATOM | 1885 | CD | PRO | 1074 | 17.565 | 14.076 | 13.978 | 1.00 | 20.42 |
| ATOM | 1886 | CA | PRO | 1074 | 17.863 | 11.709 | 14.455 | 1.00 | 23.55 |
| ATOM | 1887 | CB | PRO | 1074 | 17.364 | 11.836 | 13.038 | 1.00 | 22.58 |
| ATOM | 1888 | CG | PRO | 1074 | 16.638 | 13.184 | 13.148 | 1.00 | 23.99 |
| ATOM | 1889 | C | PRO | 1074 | 19.169 | 10.895 | 14.531 | 1.00 | 21.85 |
| ATOM | 1890 | O | PRO | 1074 | 19.159 | 9.740 | 14.954 | 1.00 | 21.99 |
| ATOM | 1891 | N | GLU | 1075 | 20.334 | 11.456 | 14.162 | 1.00 | 23.60 |
| ATOM | 1892 | H | GLU | 1075 | 20.305 | 12.319 | 13.710 | 1.00 | 0.00 |
| ATOM | 1893 | CA | GLU | 1075 | 21.624 | 10.693 | 14.253 | 1.00 | 22.19 |
| ATOM | 1894 | CB | GLU | 1075 | 22.803 | 11.479 | 13.760 | 1.00 | 20.32 |
| ATOM | 1895 | CG | GLU | 1075 | 23.127 | 12.769 | 14.544 | 1.00 | 21.36 |
| ATOM | 1896 | CD | GLU | 1075 | 22.431 | 14.040 | 14.069 | 1.00 | 21.75 |
| ATOM | 1897 | OE1 | GLU | 1075 | 21.488 | 14.002 | 13.279 | 1.00 | 19.07 |
| ATOM | 1898 | OE2 | GLU | 1075 | 22.832 | 15.098 | 14.568 | 1.00 | 24.22 |
| ATOM | 1899 | C | GLU | 1075 | 21.936 | 10.311 | 15.675 | 1.00 | 22.70 |
| ATOM | 1900 | O | GLU | 1075 | 22.677 | 9.366 | 15.939 | 1.00 | 23.48 |
| ATOM | 1901 | N | THR | 1076 | 21.375 | 11.040 | 16.656 | 1.00 | 23.21 |
| ATOM | 1902 | H | THR | 1076 | 20.855 | 11.834 | 16.429 | 1.00 | 0.00 |
| ATOM | 1903 | CA | THR | 1076 | 21.632 | 10.699 | 18.075 | 1.00 | 23.57 |
| ATOM | 1904 | CB | THR | 1076 | 21.383 | 11.786 | 19.166 | 1.00 | 19.45 |
| ATOM | 1905 | OG1 | THR | 1076 | 21.992 | 13.031 | 18.845 | 1.00 | 19.42 |
| ATOM | 1906 | HG1 | THR | 1076 | 22.124 | 13.521 | 19.663 | 1.00 | 0.00 |
| ATOM | 1907 | CG2 | THR | 1076 | 22.104 | 11.338 | 20.435 | 1.00 | 15.83 |
| ATOM | 1908 | C | THR | 1076 | 20.718 | 9.613 | 18.464 | 1.00 | 25.46 |
| ATOM | 1909 | O | THR | 1076 | 21.115 | 8.707 | 19.183 | 1.00 | 28.41 |
| ATOM | 1910 | N | ILE | 1077 | 19.491 | 9.729 | 18.036 | 1.00 | 24.96 |
| ATOM | 1911 | H | ILE | 1077 | 19.213 | 10.492 | 17.489 | 1.00 | 0.00 |
| ATOM | 1912 | CA | ILE | 1077 | 18.528 | 8.692 | 18.379 | 1.00 | 27.09 |
| ATOM | 1913 | CB | ILE | 1077 | 17.066 | 9.149 | 17.858 | 1.00 | 26.63 |
| ATOM | 1914 | CG2 | ILE | 1077 | 16.141 | 7.903 | 17.957 | 1.00 | 25.07 |
| ATOM | 1915 | CG1 | ILE | 1077 | 16.424 | 10.323 | 18.636 | 1.00 | 19.95 |
| ATOM | 1916 | CD1 | ILE | 1077 | 15.035 | 10.660 | 17.925 | 1.00 | 15.46 |
| ATOM | 1917 | C | ILE | 1077 | 19.034 | 7.372 | 17.714 | 1.00 | 26.63 |
| ATOM | 1918 | O | ILE | 1077 | 19.262 | 6.357 | 18.366 | 1.00 | 26.84 |
| ATOM | 1919 | N | PHE | 1078 | 19.345 | 7.425 | 16.439 | 1.00 | 25.14 |
| ATOM | 1920 | H | PHE | 1078 | 19.220 | 8.263 | 15.973 | 1.00 | 0.00 |
| ATOM | 1921 | CA | PHE | 1078 | 19.766 | 6.233 | 15.751 | 1.00 | 27.88 |
| ATOM | 1922 | CB | PHE | 1078 | 19.279 | 6.411 | 14.275 | 1.00 | 32.62 |
| ATOM | 1923 | CG | PHE | 1078 | 17.791 | 6.541 | 14.205 | 1.00 | 36.90 |
| ATOM | 1924 | CD1 | PHE | 1078 | 17.197 | 7.737 | 13.753 | 1.00 | 39.55 |
| ATOM | 1925 | CD2 | PHE | 1078 | 16.926 | 5.502 | 14.631 | 1.00 | 41.16 |
| ATOM | 1926 | CE1 | PHE | 1078 | 15.792 | 7.920 | 13.721 | 1.00 | 37.48 |
| ATOM | 1927 | CE2 | PHE | 1078 | 15.518 | 5.675 | 14.603 | 1.00 | 40.05 |
| ATOM | 1928 | CZ | PHE | 1078 | 14.971 | 6.884 | 14.148 | 1.00 | 38.54 |
| ATOM | 1929 | C | PHE | 1078 | 21.202 | 5.800 | 15.832 | 1.00 | 27.08 |
| ATOM | 1930 | O | PHE | 1078 | 21.453 | 4.622 | 15.908 | 1.00 | 27.60 |
| ATOM | 1931 | N | ASP | 1079 | 22.169 | 6.661 | 15.671 | 1.00 | 26.06 |
| ATOM | 1932 | H | ASP | 1079 | 21.942 | 7.595 | 15.503 | 1.00 | 0.00 |
| ATOM | 1933 | CA | ASP | 1079 | 23.575 | 6.254 | 15.739 | 1.00 | 27.56 |
| ATOM | 1934 | CB | ASP | 1079 | 24.274 | 6.894 | 14.489 | 1.00 | 32.19 |
| ATOM | 1935 | CG | ASP | 1079 | 23.442 | 6.694 | 13.181 | 1.00 | 37.46 |
| ATOM | 1936 | OD1 | ASP | 1079 | 22.787 | 5.652 | 13.086 | 1.00 | 36.79 |
| ATOM | 1937 | OD2 | ASP | 1079 | 23.457 | 7.525 | 12.243 | 1.00 | 38.85 |
| ATOM | 1938 | C | ASP | 1079 | 24.316 | 6.600 | 17.079 | 1.00 | 28.48 |
| ATOM | 1939 | O | ASP | 1079 | 25.493 | 6.319 | 17.254 | 1.00 | 27.04 |
| ATOM | 1940 | N | ASP | 1080 | 23.606 | 7.153 | 18.072 | 1.00 | 27.11 |
| ATOM | 1941 | H | ARG | 1080 | 22.630 | 7.199 | 17.979 | 1.00 | 0.00 |
| ATOM | 1942 | CA | ARG | 1080 | 24.139 | 7.594 | 19.375 | 1.00 | 27.50 |
| ATOM | 1943 | CB | ARG | 1080 | 24.623 | 6.403 | 20.204 | 1.00 | 28.96 |
| ATOM | 1944 | CG | ARG | 1080 | 23.486 | 5.450 | 20.597 | 1.00 | 32.78 |
| ATOM | 1945 | CD | ARG | 1080 | 24.070 | 4.151 | 21.218 | 1.00 | 43.85 |
| ATOM | 1946 | NE | ARG | 1080 | 25.063 | 3.597 | 20.251 | 1.00 | 54.33 |
| ATOM | 1947 | HE | ARG | 1080 | 25.479 | 4.179 | 19.583 | 1.00 | 0.00 |
| ATOM | 1948 | CZ | ARG | 1080 | 25.420 | 2.318 | 20.202 | 1.00 | 57.44 |
| ATOM | 1949 | NH1 | ARG | 1080 | 26.299 | 1.977 | 19.283 | 1.00 | 60.49 |
| ATOM | 1950 | HH11 | ARG | 1080 | 26.700 | 2.613 | 18.626 | 1.00 | 0.00 |
| ATOM | 1951 | HH12 | ARG | 1080 | 26.551 | 1.007 | 19.278 | 1.00 | 0.00 |
| ATOM | 1952 | NH2 | ARG | 1080 | 24.904 | 1.383 | 21.023 | 1.00 | 59.93 |
| ATOM | 1953 | HH21 | ARG | 1080 | 24.226 | 1.675 | 21.700 | 1.00 | 0.00 |
| ATOM | 1954 | HH22 | ARG | 1080 | 25.168 | 0.421 | 20.992 | 1.00 | 0.00 |
| ATOM | 1955 | C | ARG | 1080 | 25.262 | 8.568 | 19.134 | 1.00 | 24.40 |
| ATOM | 1956 | O | ARG | 1080 | 26.249 | 8.645 | 19.828 | 1.00 | 24.27 |
| ATOM | 1957 | N | VAL | 1081 | 25.118 | 9.378 | 18.104 | 1.00 | 23.35 |
| ATOM | 1958 | H | VAL | 1081 | 24.314 | 9.283 | 17.559 | 1.00 | 0.00 |
| ATOM | 1959 | CA | VAL | 1081 | 26.125 | 10.368 | 17.770 | 1.00 | 23.21 |
| ATOM | 1960 | CB | VAL | 1081 | 26.339 | 10.358 | 16.208 | 1.00 | 23.90 |
| ATOM | 1961 | CG1 | VAL | 1081 | 26.627 | 11.727 | 15.559 | 1.00 | 19.92 |
| ATOM | 1962 | CG2 | VAL | 1081 | 27.608 | 9.452 | 15.999 | 1.00 | 22.07 |
| ATOM | 1963 | C | VAL | 1081 | 25.776 | 11.778 | 18.295 | 1.00 | 23.54 |
| ATOM | 1964 | O | VAL | 1081 | 24.735 | 12.372 | 17.963 | 1.00 | 22.15 |
| ATOM | 1965 | N | TYR | 1082 | 26.646 | 12.235 | 19.208 | 1.00 | 21.74 |
| ATOM | 1966 | H | TYR | 1082 | 27.318 | 11.564 | 19.453 | 1.00 | 0.00 |
| ATOM | 1967 | CA | TYR | 1082 | 26.589 | 13.590 | 19.826 | 1.00 | 22.77 |
| ATOM | 1968 | CB | TYR | 1082 | 26.759 | 13.579 | 21.384 | 1.00 | 22.90 |
| ATOM | 1969 | CG | TYR | 1082 | 25.692 | 12.820 | 22.079 | 1.00 | 25.39 |
| ATOM | 1970 | CD1 | TYR | 1082 | 25.720 | 11.410 | 22.098 | 1.00 | 28.44 |
| ATOM | 1971 | CE1 | TYR | 1082 | 24.659 | 10.676 | 22.694 | 1.00 | 29.14 |
| ATOM | 1972 | CD2 | TYR | 1082 | 24.610 | 13.479 | 22.671 | 1.00 | 23.55 |
| ATOM | 1973 | CE2 | TYR | 1082 | 23.564 | 12.769 | 23.260 | 1.00 | 22.41 |
| ATOM | 1974 | CZ | TYR | 1082 | 23.579 | 11.372 | 23.270 | 1.00 | 26.90 |
| ATOM | 1975 | OH | TYR | 1082 | 22.514 | 10.661 | 23.778 | 1.00 | 30.24 |
| ATOM | 1976 | HH | TYR | 1082 | 21.928 | 11.269 | 24.218 | 1.00 | 0.00 |
| ATOM | 1977 | C | TYR | 1082 | 27.684 | 14.601 | 19.266 | 1.00 | 24.37 |
| ATOM | 1978 | O | TYR | 1082 | 28.915 | 14.394 | 19.337 | 1.00 | 24.38 |
| ATOM | 1979 | N | THR | 1083 | 27.241 | 15.707 | 18.585 | 1.00 | 23.76 |
| ATOM | 1980 | H | THR | 1083 | 26.270 | 15.830 | 18.548 | 1.00 | 0.00 |
| ATOM | 1981 | CA | THR | 1083 | 28.141 | 16.786 | 18.066 | 1.00 | 21.86 |
| ATOM | 1982 | CB | THR | 1083 | 28.288 | 16.751 | 16.513 | 1.00 | 23.57 |
| ATOM | 1983 | OG1 | THR | 1083 | 27.061 | 17.222 | 15.912 | 1.00 | 23.72 |
| ATOM | 1984 | HG1 | THR | 1083 | 27.054 | 17.083 | 14.941 | 1.00 | 0.00 |
| ATOM | 1985 | CG2 | THR | 1083 | 28.725 | 15.326 | 16.046 | 1.00 | 26.03 |
| ATOM | 1986 | C | THR | 1083 | 27.548 | 18.181 | 18.423 | 1.00 | 18.78 |
| ATOM | 1987 | O | THR | 1083 | 26.408 | 18.221 | 18.881 | 1.00 | 15.59 |
| ATOM | 1988 | N | ILE | 1084 | 28.156 | 19.365 | 18.283 | 1.00 | 16.71 |
| ATOM | 1989 | H | ILE | 1084 | 29.069 | 19.389 | 17.928 | 1.00 | 0.00 |
| ATOM | 1990 | CA | ILE | 1084 | 27.361 | 20.581 | 18.654 | 1.00 | 18.90 |
| ATOM | 1991 | CB | ILE | 1084 | 28.060 | 22.038 | 18.644 | 1.00 | 22.05 |
| ATOM | 1992 | CG2 | ILE | 1084 | 28.095 | 22.769 | 20.037 | 1.00 | 15.28 |
| ATOM | 1993 | CG1 | ILE | 1084 | 29.402 | 21.825 | 17.997 | 1.00 | 25.17 |
| ATOM | 1994 | CD1 | ILE | 1084 | 29.305 | 21.556 | 16.484 | 1.00 | 26.38 |
| ATOM | 1995 | C | ILE | 1084 | 26.260 | 20.695 | 17.648 | 1.00 | 17.38 |
| ATOM | 1996 | O | ILE | 1084 | 25.253 | 21.358 | 17.868 | 1.00 | 15.83 |
| ATOM | 1997 | N | GLN | 1085 | 26.401 | 20.000 | 16.520 | 1.00 | 17.70 |
| ATOM | 1998 | H | GLN | 1085 | 27.206 | 19.470 | 16.388 | 1.00 | 0.00 |
| ATOM | 1999 | CA | GLN | 1085 | 25.312 | 20.065 | 15.491 | 1.00 | 18.91 |
| ATOM | 2000 | CB | GLN | 1085 | 25.762 | 19.462 | 14.142 | 1.00 | 18.78 |
| ATOM | 2001 | CG | GLN | 1085 | 26.721 | 20.542 | 13.476 | 1.00 | 20.74 |
| ATOM | 2002 | CD | GLN | 1085 | 26.266 | 22.037 | 13.610 | 1.00 | 22.47 |
| ATOM | 2003 | OE1 | GLN | 1085 | 26.884 | 22.836 | 14.325 | 1.00 | 21.93 |
| ATOM | 2004 | NE2 | GLN | 1085 | 25.232 | 22.502 | 12.900 | 1.00 | 22.20 |
| ATOM | 2005 | HE21 | GLN | 1085 | 24.716 | 21.950 | 12.273 | 1.00 | 0.00 |
| ATOM | 2006 | HE22 | GLN | 1085 | 24.990 | 23.442 | 13.028 | 1.00 | 0.00 |
| ATOM | 2007 | C | GLN | 1085 | 24.090 | 19.381 | 15.974 | 1.00 | 16.52 |
| ATOM | 2008 | O | GLN | 1085 | 22.970 | 19.765 | 15.632 | 1.00 | 18.26 |
| ATOM | 2009 | N | SER | 1086 | 24.292 | 18.243 | 16.646 | 1.00 | 17.01 |
| ATOM | 2010 | H | SER | 1086 | 25.187 | 17.884 | 16.826 | 1.00 | 0.00 |
| ATOM | 2011 | CA | SER | 1086 | 23.091 | 17.621 | 17.228 | 1.00 | 16.18 |
| ATOM | 2012 | CB | SER | 1086 | 23.338 | 16.148 | 17.786 | 1.00 | 14.38 |
| ATOM | 2013 | OG | SER | 1086 | 24.275 | 15.939 | 18.801 | 1.00 | 16.17 |
| ATOM | 2014 | HG | SER | 1086 | 24.247 | 14.994 | 19.025 | 1.00 | 0.00 |
| ATOM | 2015 | C | SER | 1086 | 22.683 | 18.652 | 18.347 | 1.00 | 16.61 |
| ATOM | 2016 | O | SER | 1086 | 21.483 | 18.879 | 18.560 | 1.00 | 15.97 |
| ATOM | 2017 | N | ASP | 1087 | 23.604 | 19.340 | 19.086 | 1.00 | 14.52 |
| ATOM | 2018 | H | ASP | 1087 | 24.547 | 19.060 | 19.040 | 1.00 | 0.00 |
| ATOM | 2019 | CA | ASP | 1087 | 23.103 | 20.384 | 20.018 | 1.00 | 14.62 |
| ATOM | 2020 | CB | ASP | 1087 | 24.229 | 21.219 | 20.762 | 1.00 | 11.48 |
| ATOM | 2021 | CG | ASP | 1087 | 24.799 | 20.469 | 21.959 | 1.00 | 12.91 |
| ATOM | 2022 | OD1 | ASP | 1087 | 25.850 | 20.862 | 22.435 | 1.00 | 19.23 |
| ATOM | 2023 | OD2 | ASP | 1087 | 24.198 | 19.517 | 22.447 | 1.00 | 17.19 |
| ATOM | 2024 | C | ASP | 1087 | 22.256 | 21.431 | 19.230 | 1.00 | 17.48 |
| ATOM | 2025 | O | ASP | 1087 | 21.257 | 21.932 | 19.787 | 1.00 | 19.47 |
| ATOM | 2026 | N | VAL | 1088 | 22.610 | 21.841 | 17.962 | 1.00 | 16.08 |
| ATOM | 2027 | H | VAL | 1088 | 23.415 | 21.485 | 17.544 | 1.00 | 0.00 |
| ATOM | 2028 | CA | VAL | 1088 | 21.761 | 22.829 | 17.220 | 1.00 | 14.51 |
| ATOM | 2029 | CB | VAL | 1088 | 22.379 | 23.224 | 15.866 | 1.00 | 14.37 |
| ATOM | 2030 | CG1 | VAL | 1088 | 21.398 | 24.062 | 15.066 | 1.00 | 8.75 |
| ATOM | 2031 | CG2 | VAL | 1088 | 23.589 | 24.149 | 16.116 | 1.00 | 8.08 |
| ATOM | 2032 | C | VAL | 1088 | 20.383 | 22.294 | 17.011 | 1.00 | 14.71 |
| ATOM | 2033 | O | VAL | 1088 | 19.447 | 23.109 | 17.063 | 1.00 | 14.59 |
| ATOM | 2034 | N | TRP | 1089 | 20.215 | 20.956 | 16.760 | 1.00 | 13.99 |
| ATOM | 2035 | H | TRP | 1089 | 21.009 | 20.398 | 16.618 | 1.00 | 0.00 |
| ATOM | 2036 | CA | TRP | 1089 | 18.837 | 20.393 | 16.657 | 1.00 | 11.59 |
| ATOM | 2037 | CB | TRP | 1089 | 18.952 | 18.881 | 16.353 | 1.00 | 11.21 |
| ATOM | 2038 | CG | TRP | 1089 | 17.623 | 18.190 | 16.309 | 1.00 | 11.56 |
| ATOM | 2039 | CD2 | TRP | 1089 | 16.849 | 17.711 | 15.207 | 1.00 | 10.09 |
| ATOM | 2040 | CE2 | TRP | 1089 | 15.611 | 17.248 | 15.697 | 1.00 | 9.26 |
| ATOM | 2041 | CE3 | TRP | 1089 | 17.088 | 17.640 | 13.829 | 1.00 | 11.56 |
| ATOM | 2042 | CD1 | TRP | 1089 | 16.779 | 17.971 | 17.424 | 1.00 | 12.86 |
| ATOM | 2043 | NE1 | TRP | 1089 | 15.570 | 17.426 | 17.039 | 1.00 | 12.10 |
| ATOM | 2044 | HE1 | TRP | 1089 | 14.892 | 17.112 | 17.684 | 1.00 | 0.00 |
| ATOM | 2045 | CZ2 | TRP | 1089 | 14.663 | 16.761 | 14.833 | 1.00 | 10.88 |
| ATOM | 2046 | CZ3 | TRP | 1089 | 16.099 | 17.126 | 12.969 | 1.00 | 10.50 |
| ATOM | 2047 | CH2 | TRP | 1089 | 14.903 | 16.691 | 13.469 | 1.00 | 10.61 |
| ATOM | 2048 | C | TRP | 1089 | 18.034 | 20.627 | 17.997 | 1.00 | 12.12 |
| ATOM | 2049 | O | TRP | 1089 | 16.916 | 21.119 | 17.980 | 1.00 | 15.29 |
| ATOM | 2050 | N | SER | 1090 | 18.588 | 20.303 | 19.201 | 1.00 | 15.20 |
| ATOM | 2051 | H | SER | 1090 | 19.464 | 19.867 | 19.140 | 1.00 | 0.00 |
| ATOM | 2052 | CA | SER | 1090 | 17.965 | 20.475 | 20.566 | 1.00 | 17.83 |
| ATOM | 2053 | CB | SER | 1090 | 18.875 | 20.128 | 21.777 | 1.00 | 19.05 |
| ATOM | 2054 | OG | SER | 1090 | 19.368 | 18.785 | 21.708 | 1.00 | 16.80 |
| ATOM | 2055 | HG | SER | 1090 | 19.426 | 18.465 | 22.608 | 1.00 | 0.00 |
| ATOM | 2056 | C | SER | 1090 | 17.608 | 21.952 | 20.766 | 1.00 | 17.91 |
| ATOM | 2057 | O | SER | 1090 | 16.563 | 22.347 | 21.295 | 1.00 | 16.63 |
| ATOM | 2058 | N | PHE | 1091 | 18.506 | 22.850 | 20.306 | 1.00 | 18.25 |
| ATOM | 2059 | H | PHE | 1091 | 19.345 | 22.490 | 19.956 | 1.00 | 0.00 |
| ATOM | 2060 | CA | PHE | 1091 | 18.302 | 24.321 | 20.334 | 1.00 | 12.73 |
| ATOM | 2061 | CB | PHE | 1091 | 19.593 | 25.044 | 19.795 | 1.00 | 12.19 |
| ATOM | 2062 | CG | PHE | 1091 | 19.416 | 26.499 | 19.863 | 1.00 | 15.22 |
| ATOM | 2063 | CD1 | PHE | 1091 | 19.709 | 27.188 | 21.069 | 1.00 | 14.01 |
| ATOM | 2064 | CD2 | PHE | 1091 | 18.913 | 27.207 | 18.722 | 1.00 | 17.10 |
| ATOM | 2065 | CE1 | PHE | 1091 | 19.482 | 28.577 | 21.115 | 1.00 | 10.32 |
| ATOM | 2066 | CE2 | PHE | 1091 | 18.697 | 28.581 | 18.800 | 1.00 | 13.58 |
| ATOM | 2067 | CZ | PHE | 1091 | 18.990 | 29.244 | 19.991 | 1.00 | 10.87 |
| ATOM | 2068 | C | PHE | 1091 | 17.050 | 24.649 | 19.474 | 1.00 | 12.77 |
| ATOM | 2069 | O | PHE | 1091 | 16.241 | 25.480 | 19.894 | 1.00 | 13.31 |
| ATOM | 2070 | N | GLY | 1092 | 16.806 | 24.116 | 18.274 | 1.00 | 8.99 |
| ATOM | 2071 | H | GLY | 1092 | 17.505 | 23.556 | 17.865 | 1.00 | 0.00 |
| ATOM | 2072 | CA | GLY | 1092 | 15.523 | 24.419 | 17.552 | 1.00 | 10.24 |
| ATOM | 2073 | C | GLY | 1092 | 14.342 | 23.876 | 18.402 | 1.00 | 9.34 |
| ATOM | 2074 | O | GLY | 1092 | 13.208 | 24.382 | 18.429 | 1.00 | 10.83 |
| ATOM | 2075 | N | VAL | 1093 | 14.515 | 22.772 | 19.128 | 1.00 | 10.21 |
| ATOM | 2076 | H | VAL | 1093 | 15.362 | 22.279 | 19.099 | 1.00 | 0.00 |
| ATOM | 2077 | CA | VAL | 1093 | 13.395 | 22.318 | 19.975 | 1.00 | 11.40 |
| ATOM | 2078 | CB | VAL | 1093 | 13.653 | 20.864 | 20.602 | 1.00 | 14.77 |
| ATOM | 2079 | CG1 | VAL | 1093 | 12.447 | 20.388 | 21.490 | 1.00 | 9.80 |
| ATOM | 2080 | CG2 | VAL | 1093 | 13.768 | 19.806 | 19.462 | 1.00 | 14.64 |
| ATOM | 2081 | C | VAL | 1093 | 13.212 | 23.349 | 21.117 | 1.00 | 11.93 |
| ATOM | 2082 | O | VAL | 1093 | 12.089 | 23.676 | 21.568 | 1.00 | 14.37 |
| ATOM | 2083 | N | LEU | 1094 | 14.317 | 23.906 | 21.618 | 1.00 | 12.53 |
| ATOM | 2084 | H | LEU | 1094 | 15.170 | 23.593 | 21.261 | 1.00 | 0.00 |
| ATOM | 2085 | CA | LEU | 1094 | 14.257 | 24.882 | 22.717 | 1.00 | 13.31 |
| ATOM | 2086 | CB | LEU | 1094 | 15.687 | 25.172 | 23.147 | 1.00 | 12.05 |
| ATOM | 2087 | CG | LEU | 1094 | 15.950 | 25.855 | 24.504 | 1.00 | 16.08 |
| ATOM | 2088 | CD1 | LEU | 1094 | 16.915 | 26.915 | 24.123 | 1.00 | 16.44 |
| ATOM | 2089 | CD2 | LEU | 1094 | 14.913 | 26.830 | 25.092 | 1.00 | 19.40 |
| ATOM | 2090 | C | LEU | 1094 | 13.509 | 26.137 | 22.211 | 1.00 | 16.37 |
| ATOM | 2091 | O | LEU | 1094 | 12.644 | 26.673 | 22.928 | 1.00 | 18.28 |
| ATOM | 2092 | N | LEU | 1095 | 13.809 | 26.597 | 20.978 | 1.00 | 13.21 |
| ATOM | 2093 | H | LEU | 1095 | 14.557 | 26.151 | 20.528 | 1.00 | 0.00 |
| ATOM | 2094 | CA | LEU | 1095 | 13.133 | 27.772 | 20.341 | 1.00 | 15.04 |
| ATOM | 2095 | CB | LEU | 1095 | 13.614 | 27.938 | 18.886 | 1.00 | 15.20 |
| ATOM | 2096 | CG | LEU | 1095 | 15.019 | 28.467 | 18.677 | 1.00 | 16.50 |
| ATOM | 2097 | CD1 | LEU | 1095 | 15.304 | 28.509 | 17.140 | 1.00 | 15.08 |
| ATOM | 2098 | CD2 | LEU | 1095 | 15.150 | 29.832 | 19.344 | 1.00 | 13.08 |
| ATOM | 2099 | C | LEU | 1095 | 11.630 | 27.474 | 20.323 | 1.00 | 15.20 |
| ATOM | 2100 | O | LEU | 1095 | 10.793 | 28.293 | 20.725 | 1.00 | 13.91 |
| ATOM | 2101 | N | TRP | 1096 | 11.270 | 26.240 | 19.850 | 1.00 | 14.00 |
| ATOM | 2102 | H | TRP | 1096 | 11.956 | 25.637 | 19.475 | 1.00 | 0.00 |
| ATOM | 2103 | CA | TRP | 1096 | 9.857 | 25.823 | 19.856 | 1.00 | 13.63 |
| ATOM | 2104 | CB | TRP | 1096 | 9.658 | 24.338 | 19.420 | 1.00 | 11.90 |
| ATOM | 2105 | CG | TRP | 1096 | 8.198 | 24.046 | 19.170 | 1.00 | 14.13 |
| ATOM | 2106 | CD2 | TRP | 1096 | 7.252 | 23.627 | 20.137 | 1.00 | 14.10 |
| ATOM | 2107 | CE2 | TRP | 1096 | 5.991 | 23.463 | 19.443 | 1.00 | 13.99 |
| ATOM | 2108 | CE3 | TRP | 1096 | 7.394 | 23.327 | 21.528 | 1.00 | 15.07 |
| ATOM | 2109 | CD1 | TRP | 1096 | 7.436 | 24.150 | 17.941 | 1.00 | 14.08 |
| ATOM | 2110 | NE1 | TRP | 1096 | 6.142 | 23.771 | 18.097 | 1.00 | 10.60 |
| ATOM | 2111 | HE1 | TRP | 1096 | 5.438 | 23.837 | 17.410 | 1.00 | 0.00 |
| ATOM | 2112 | CZ2 | TRP | 1096 | 4.888 | 23.006 | 20.227 | 1.00 | 16.17 |
| ATOM | 2113 | CZ3 | TRP | 1096 | 6.281 | 22.902 | 22.241 | 1.00 | 14.56 |
| ATOM | 2114 | CH2 | TRP | 1096 | 5.030 | 22.738 | 21.613 | 1.00 | 14.16 |
| ATOM | 2115 | C | TRP | 1096 | 9.335 | 25.952 | 21.338 | 1.00 | 14.07 |
| ATOM | 2116 | O | TRP | 1096 | 8.234 | 26.481 | 21.553 | 1.00 | 17.28 |
| ATOM | 2117 | N | GLU | 1097 | 10.079 | 25.479 | 22.384 | 1.00 | 12.66 |
| ATOM | 2118 | H | GLU | 1097 | 10.915 | 25.020 | 22.173 | 1.00 | 0.00 |
| ATOM | 2119 | CA | GLU | 1097 | 9.624 | 25.639 | 23.791 | 1.00 | 12.86 |
| ATOM | 2120 | CB | GLU | 1097 | 10.622 | 24.968 | 24.781 | 1.00 | 10.84 |
| ATOM | 2121 | CG | GLU | 1097 | 10.592 | 23.453 | 24.631 | 1.00 | 11.95 |
| ATOM | 2122 | CD | GLU | 1097 | 11.576 | 22.932 | 25.655 | 1.00 | 15.90 |
| ATOM | 2123 | OE1 | GLU | 1097 | 11.168 | 22.521 | 26.755 | 1.00 | 16.96 |
| ATOM | 2124 | OE2 | GLU | 1097 | 12.756 | 22.959 | 25.356 | 1.00 | 12.11 |
| ATOM | 2125 | C | GLU | 1097 | 9.419 | 27.111 | 24.241 | 1.00 | 12.63 |
| ATOM | 2126 | O | GLU | 1097 | 8.474 | 27.489 | 24.934 | 1.00 | 11.38 |
| ATOM | 2127 | N | ILE | 1098 | 10.320 | 27.986 | 23.816 | 1.00 | 12.74 |
| ATOM | 2128 | H | ILE | 1098 | 11.033 | 27.658 | 23.230 | 1.00 | 0.00 |
| ATOM | 2129 | CA | ILE | 1098 | 10.211 | 29.391 | 24.167 | 1.00 | 12.52 |
| ATOM | 2130 | CB | ILE | 1098 | 11.533 | 30.065 | 23.775 | 1.00 | 13.95 |
| ATOM | 2131 | CG2 | ILE | 1098 | 11.222 | 31.584 | 23.724 | 1.00 | 13.20 |
| ATOM | 2132 | CG1 | ILE | 1098 | 12.680 | 29.556 | 24.634 | 1.00 | 10.22 |
| ATOM | 2133 | CD1 | ILE | 1098 | 14.056 | 30.062 | 24.152 | 1.00 | 12.21 |
| ATOM | 2134 | C | ILE | 1098 | 9.027 | 30.027 | 23.461 | 1.00 | 14.43 |
| ATOM | 2135 | O | ILE | 1098 | 8.196 | 30.709 | 24.101 | 1.00 | 15.21 |
| ATOM | 2136 | N | PHE | 1099 | 8.919 | 29.830 | 22.119 | 1.00 | 11.42 |
| ATOM | 2137 | H | PHE | 1099 | 9.598 | 29.293 | 21.673 | 1.00 | 0.00 |
| ATOM | 2138 | CA | PHE | 1099 | 7.817 | 30.468 | 21.419 | 1.00 | 11.95 |
| ATOM | 2139 | CB | PHE | 1099 | 8.251 | 30.695 | 19.975 | 1.00 | 16.00 |
| ATOM | 2140 | CG | PHE | 1099 | 9.353 | 31.680 | 19.974 | 1.00 | 13.92 |
| ATOM | 2141 | CD1 | PHE | 1099 | 10.653 | 31.275 | 20.218 | 1.00 | 12.17 |
| ATOM | 2142 | CD2 | PHE | 1099 | 9.055 | 33.017 | 19.721 | 1.00 | 14.65 |
| ATOM | 2143 | CE1 | PHE | 1099 | 11.672 | 32.221 | 20.202 | 1.00 | 14.43 |
| ATOM | 2144 | CE2 | PHE | 1099 | 10.092 | 33.968 | 19.696 | 1.00 | 18.96 |
| ATOM | 2145 | CZ | PHE | 1099 | 11.421 | 33.563 | 19.942 | 1.00 | 13.07 |
| ATOM | 2146 | C | PHE | 1099 | 6.492 | 29.801 | 21.500 | 1.00 | 14.19 |
| ATOM | 2147 | O | PHE | 1099 | 5.558 | 30.095 | 20.765 | 1.00 | 16.64 |
| ATOM | 2148 | N | SER | 1100 | 6.327 | 28.833 | 22.384 | 1.00 | 13.41 |
| ATOM | 2149 | H | SER | 1100 | 7.146 | 28.453 | 22.758 | 1.00 | 0.00 |
| ATOM | 2150 | CA | SER | 1100 | 5.033 | 28.160 | 22.690 | 1.00 | 12.04 |
| ATOM | 2151 | CB | SER | 1100 | 5.112 | 26.630 | 22.535 | 1.00 | 13.80 |
| ATOM | 2152 | OG | SER | 1100 | 6.010 | 26.121 | 23.530 | 1.00 | 15.15 |
| ATOM | 2153 | HG | SER | 1100 | 6.203 | 25.196 | 23.323 | 1.00 | 0.00 |
| ATOM | 2154 | C | SER | 1100 | 4.727 | 28.471 | 24.180 | 1.00 | 12.57 |
| ATOM | 2155 | O | SER | 1100 | 3.679 | 28.173 | 24.725 | 1.00 | 13.35 |
| ATOM | 2156 | N | LEU | 1101 | 5.606 | 29.199 | 24.863 | 1.00 | 13.95 |
| ATOM | 2157 | H | LEU | 1101 | 6.359 | 29.548 | 24.347 | 1.00 | 0.00 |
| ATOM | 2158 | CA | LEU | 1101 | 5.587 | 29.606 | 26.292 | 1.00 | 16.11 |
| ATOM | 2159 | CB | LEU | 1101 | 4.386 | 30.603 | 26.683 | 1.00 | 15.50 |
| ATOM | 2160 | CG | LEU | 1101 | 4.474 | 32.042 | 25.977 | 1.00 | 19.92 |
| ATOM | 2161 | CD1 | LEU | 1101 | 3.347 | 33.042 | 26.469 | 1.00 | 15.10 |
| ATOM | 2162 | CD2 | LEU | 1101 | 5.919 | 32.605 | 26.217 | 1.00 | 18.71 |
| ATOM | 2163 | C | LEU | 1101 | 5.578 | 28.414 | 27.227 | 1.00 | 18.97 |
| ATOM | 2164 | O | LEU | 1101 | 4.656 | 28.121 | 27.993 | 1.00 | 19.74 |
| ATOM | 2165 | N | GLY | 1102 | 6.713 | 27.663 | 27.154 | 1.00 | 18.82 |
| ATOM | 2166 | H | GLY | 1102 | 7.391 | 27.915 | 26.497 | 1.00 | 0.00 |
| ATOM | 2167 | CA | GLY | 1102 | 6.932 | 26.468 | 28.008 | 1.00 | 18.72 |
| ATOM | 2168 | C | GLY | 1102 | 6.060 | 25.273 | 27.663 | 1.00 | 19.72 |
| ATOM | 2169 | O | GLY | 1102 | 5.789 | 24.427 | 28.499 | 1.00 | 20.31 |
| ATOM | 2170 | N | ALA | 1103 | 5.520 | 25.170 | 26.446 | 1.00 | 20.13 |
| ATOM | 2171 | H | ALA | 1103 | 5.634 | 25.909 | 25.814 | 1.00 | 0.00 |
| ATOM | 2172 | CA | ALA | 1103 | 4.753 | 23.968 | 26.112 | 1.00 | 17.88 |
| ATOM | 2173 | CB | ALA | 1103 | 3.914 | 24.102 | 24.817 | 1.00 | 15.91 |
| ATOM | 2174 | C | ALA | 1103 | 5.683 | 22.772 | 25.888 | 1.00 | 17.89 |
| ATOM | 2175 | O | ALA | 1103 | 6.872 | 22.884 | 25.632 | 1.00 | 17.17 |
| ATOM | 2176 | N | SER | 1104 | 5.123 | 21.574 | 26.052 | 1.00 | 18.52 |
| ATOM | 2177 | H | SER | 1104 | 4.188 | 21.630 | 26.299 | 1.00 | 0.00 |
| ATOM | 2178 | CA | SER | 1104 | 5.819 | 20.278 | 25.842 | 1.00 | 18.30 |
| ATOM | 2179 | CB | SER | 1104 | 5.039 | 19.039 | 26.382 | 1.00 | 20.13 |
| ATOM | 2180 | OG | SER | 1104 | 5.919 | 17.975 | 26.780 | 1.00 | 26.13 |
| ATOM | 2181 | HG | SER | 1104 | 5.431 | 17.142 | 26.836 | 1.00 | 0.00 |
| ATOM | 2182 | C | SER | 1104 | 5.946 | 20.001 | 24.365 | 1.00 | 17.20 |
| ATOM | 2183 | O | SER | 1104 | 4.934 | 19.967 | 23.643 | 1.00 | 17.08 |
| ATOM | 2184 | N | PRO | 1105 | 7.130 | 19.713 | 23.863 | 1.00 | 16.63 |
| ATOM | 2185 | CD | PRO | 1105 | 8.411 | 19.593 | 24.570 | 1.00 | 14.02 |
| ATOM | 2186 | CA | PRO | 1105 | 7.273 | 19.451 | 22.425 | 1.00 | 14.80 |
| ATOM | 2187 | CB | PRO | 1105 | 8.748 | 19.151 | 22.205 | 1.00 | 14.42 |
| ATOM | 2188 | CG | PRO | 1105 | 9.319 | 19.938 | 23.358 | 1.00 | 15.62 |
| ATOM | 2189 | C | PRO | 1105 | 6.413 | 18.299 | 21.922 | 1.00 | 18.18 |
| ATOM | 2190 | O | PRO | 1105 | 6.012 | 17.404 | 22.652 | 1.00 | 20.08 |
| ATOM | 2191 | N | TYR | 1106 | 6.234 | 18.214 | 20.605 | 1.00 | 17.66 |
| ATOM | 2192 | H | TYR | 1106 | 6.677 | 18.911 | 20.078 | 1.00 | 0.00 |
| ATOM | 2193 | CA | TYR | 1106 | 5.452 | 17.162 | 19.948 | 1.00 | 17.71 |
| ATOM | 2194 | CB | TYR | 1106 | 6.269 | 15.851 | 19.891 | 1.00 | 15.59 |
| ATOM | 2195 | CG | TYR | 1106 | 7.450 | 16.069 | 19.057 | 1.00 | 16.76 |
| ATOM | 2196 | CD1 | TYR | 1106 | 8.667 | 16.343 | 19.669 | 1.00 | 14.87 |
| ATOM | 2197 | CE1 | TYR | 1106 | 9.808 | 16.602 | 18.904 | 1.00 | 15.24 |
| ATOM | 2198 | CD2 | TYR | 1106 | 7.356 | 16.043 | 17.668 | 1.00 | 17.07 |
| ATOM | 2199 | CE2 | TYR | 1106 | 8.507 | 16.296 | 16.883 | 1.00 | 14.93 |
| ATOM | 2200 | CZ | TYR | 1106 | 9.752 | 16.584 | 17.484 | 1.00 | 15.79 |
| ATOM | 2201 | OH | TYR | 1106 | 10.888 | 16.901 | 16.734 | 1.00 | 13.47 |
| ATOM | 2202 | HH | TYR | 1106 | 11.377 | 17.612 | 17.157 | 1.00 | 0.00 |
| ATOM | 2203 | C | TYR | 1106 | 4.110 | 16.918 | 20.657 | 1.00 | 20.65 |
| ATOM | 2204 | O | TYR | 1106 | 3.807 | 15.811 | 21.116 | 1.00 | 20.19 |
| ATOM | 2205 | N | PRO | 1107 | 3.272 | 17.966 | 20.727 | 1.00 | 20.84 |
| ATOM | 2206 | CD | PRO | 1107 | 3.478 | 19.332 | 20.192 | 1.00 | 20.44 |
| ATOM | 2207 | CA | PRO | 1107 | 1.956 | 17.857 | 21.314 | 1.00 | 23.78 |
| ATOM | 2208 | CB | PRO | 1107 | 1.327 | 19.173 | 21.039 | 1.00 | 22.40 |
| ATOM | 2209 | CG | PRO | 1107 | 2.092 | 19.738 | 19.819 | 1.00 | 22.72 |
| ATOM | 2210 | C | PRO | 1107 | 1.135 | 16.706 | 20.690 | 1.00 | 29.57 |
| ATOM | 2211 | O | PRO | 1107 | 1.040 | 16.592 | 19.437 | 1.00 | 28.41 |
| ATOM | 2212 | N | GLY | 1108 | 0.577 | 15.846 | 21.587 | 1.00 | 31.62 |
| ATOM | 2213 | H | GLY | 1108 | 0.774 | 16.007 | 22.533 | 1.00 | 0.00 |
| ATOM | 2214 | CA | GLY | 1108 | −0.281 | 14.668 | 21.170 | 1.00 | 37.14 |
| ATOM | 2215 | C | GLY | 1108 | 0.394 | 13.287 | 20.910 | 1.00 | 38.69 |
| ATOM | 2216 | O | GLY | 1108 | −0.053 | 12.219 | 21.349 | 1.00 | 41.30 |
| ATOM | 2217 | N | VAL | 1109 | 1.545 | 13.392 | 20.238 | 1.00 | 37.52 |
| ATOM | 2218 | H | VAL | 1109 | 1.850 | 14.319 | 20.167 | 1.00 | 0.00 |
| ATOM | 2219 | CA | VAL | 1109 | 2.446 | 12.337 | 19.787 | 1.00 | 32.09 |
| ATOM | 2220 | CB | VAL | 1109 | 3.571 | 13.054 | 19.036 | 1.00 | 31.43 |
| ATOM | 2221 | CG1 | VAL | 1109 | 4.241 | 11.935 | 18.306 | 1.00 | 35.34 |
| ATOM | 2222 | CG2 | VAL | 1109 | 3.143 | 14.224 | 18.074 | 1.00 | 28.01 |
| ATOM | 2223 | C | VAL | 1109 | 2.981 | 11.439 | 20.894 | 1.00 | 31.16 |
| ATOM | 2224 | O | VAL | 1109 | 3.392 | 11.886 | 21.960 | 1.00 | 32.64 |
| ATOM | 2225 | N | LYS | 1110 | 2.832 | 10.116 | 20.755 | 1.00 | 30.62 |
| ATOM | 2226 | H | LYS | 1110 | 2.316 | 9.806 | 19.986 | 1.00 | 0.00 |
| ATOM | 2227 | CA | LYS | 1110 | 3.398 | 9.190 | 21.774 | 1.00 | 30.39 |
| ATOM | 2228 | CB | LYS | 1110 | 2.734 | 7.716 | 21.786 | 1.00 | 30.14 |
| ATOM | 2229 | C | LYS | 1110 | 4.864 | 9.059 | 21.362 | 1.00 | 30.79 |
| ATOM | 2230 | O | LYS | 1110 | 5.205 | 9.133 | 20.170 | 1.00 | 32.22 |
| ATOM | 2231 | N | ILE | 1111 | 5.812 | 8.927 | 22.278 | 1.00 | 32.17 |
| ATOM | 2232 | H | ILE | 1111 | 5.568 | 8.951 | 23.226 | 1.00 | 0.00 |
| ATOM | 2233 | CA | ILE | 1111 | 7.230 | 8.804 | 21.851 | 1.00 | 29.12 |
| ATOM | 2234 | CB | ILE | 1111 | 8.038 | 9.732 | 22.801 | 1.00 | 23.72 |
| ATOM | 2235 | CG2 | ILE | 1111 | 9.561 | 9.795 | 22.505 | 1.00 | 22.60 |
| ATOM | 2236 | CG1 | ILE | 1111 | 7.384 | 11.118 | 22.642 | 1.00 | 20.16 |
| ATOM | 2237 | CD1 | ILE | 1111 | 7.209 | 11.715 | 21.242 | 1.00 | 21.61 |
| ATOM | 2238 | C | ILE | 1111 | 7.694 | 7.319 | 21.809 | 1.00 | 31.35 |
| ATOM | 2239 | O | ILE | 1111 | 8.185 | 6.663 | 22.746 | 1.00 | 31.59 |
| ATOM | 2240 | N | ASP | 1112 | 7.432 | 6.806 | 20.605 | 1.00 | 31.61 |
| ATOM | 2241 | H | ASP | 1112 | 7.060 | 7.463 | 19.983 | 1.00 | 0.00 |
| ATOM | 2242 | CA | ASP | 1112 | 7.676 | 5.421 | 20.161 | 1.00 | 35.69 |
| ATOM | 2243 | CB | ASP | 1112 | 6.259 | 4.753 | 19.870 | 1.00 | 38.63 |
| ATOM | 2244 | CG | ASP | 1112 | 5.449 | 5.354 | 18.699 | 1.00 | 40.37 |
| ATOM | 2245 | OD1 | ASP | 1112 | 4.306 | 4.954 | 18.439 | 1.00 | 48.79 |
| ATOM | 2246 | OD2 | ASP | 1112 | 5.940 | 6.223 | 18.005 | 1.00 | 42.10 |
| ATOM | 2247 | C | ASP | 1112 | 8.616 | 5.230 | 18.949 | 1.00 | 36.56 |
| ATOM | 2248 | O | ASP | 1112 | 9.204 | 6.210 | 18.471 | 1.00 | 35.62 |
| ATOM | 2249 | N | GLU | 1113 | 8.760 | 3.969 | 18.424 | 1.00 | 36.93 |
| ATOM | 2250 | H | GLU | 1113 | 8.364 | 3.242 | 18.941 | 1.00 | 0.00 |
| ATOM | 2251 | CA | GLU | 1113 | 9.622 | 3.669 | 17.237 | 1.00 | 36.90 |
| ATOM | 2252 | CB | GLU | 1113 | 9.772 | 2.125 | 16.946 | 1.00 | 38.25 |
| ATOM | 2253 | C | GLU | 1113 | 9.018 | 4.344 | 15.997 | 1.00 | 35.95 |
| ATOM | 2254 | O | GLU | 1113 | 9.732 | 4.749 | 15.069 | 1.00 | 36.13 |
| ATOM | 2255 | N | GLU | 1114 | 7.690 | 4.519 | 15.975 | 1.00 | 34.50 |
| ATOM | 2256 | H | GLU | 1114 | 7.155 | 4.088 | 16.671 | 1.00 | 0.00 |
| ATOM | 2257 | CA | GLU | 1114 | 7.058 | 5.253 | 14.834 | 1.00 | 34.65 |
| ATOM | 2258 | CB | GLU | 1114 | 5.547 | 4.930 | 14.888 | 1.00 | 40.80 |
| ATOM | 2259 | CG | GLU | 1114 | 5.492 | 3.392 | 14.549 | 1.00 | 50.03 |
| ATOM | 2260 | CD | GLU | 1114 | 6.325 | 3.063 | 13.267 | 1.00 | 54.57 |
| ATOM | 2261 | OE1 | GLU | 1114 | 7.308 | 2.290 | 13.334 | 1.00 | 54.49 |
| ATOM | 2262 | OE2 | GLU | 1114 | 5.972 | 3.612 | 12.210 | 1.00 | 55.11 |
| ATOM | 2263 | C | GLU | 1114 | 7.356 | 6.769 | 14.848 | 1.00 | 30.99 |
| ATOM | 2264 | O | GLU | 1114 | 7.628 | 7.421 | 13.851 | 1.00 | 27.97 |
| ATOM | 2265 | N | PHE | 1115 | 7.407 | 7.365 | 16.036 | 1.00 | 30.23 |
| ATOM | 2266 | H | PHE | 1115 | 7.429 | 6.808 | 16.828 | 1.00 | 0.00 |
| ATOM | 2267 | CA | PHE | 1115 | 7.756 | 8.786 | 16.209 | 1.00 | 27.81 |
| ATOM | 2268 | CB | PHE | 1115 | 7.628 | 9.112 | 17.692 | 1.00 | 25.50 |
| ATOM | 2269 | CG | PHE | 1115 | 8.537 | 10.210 | 18.107 | 1.00 | 26.18 |
| ATOM | 2270 | CD1 | PHE | 1115 | 9.815 | 9.933 | 18.622 | 1.00 | 24.22 |
| ATOM | 2271 | CD2 | PHE | 1115 | 8.139 | 11.539 | 17.923 | 1.00 | 23.71 |
| ATOM | 2272 | CE1 | PHE | 1115 | 10.700 | 10.973 | 18.942 | 1.00 | 24.88 |
| ATOM | 2273 | CE2 | PHE | 1115 | 9.045 | 12.568 | 18.249 | 1.00 | 24.43 |
| ATOM | 2274 | CZ | PHE | 1115 | 10.318 | 12.303 | 18.752 | 1.00 | 23.12 |
| ATOM | 2275 | C | PHE | 1115 | 9.181 | 8.871 | 15.704 | 1.00 | 27.12 |
| ATOM | 2276 | O | PHE | 1115 | 9.485 | 9.631 | 14.788 | 1.00 | 28.47 |
| ATOM | 2277 | N | CYS | 1116 | 10.084 | 8.001 | 16.142 | 1.00 | 27.10 |
| ATOM | 2278 | H | CYS | 1116 | 9.851 | 7.365 | 16.850 | 1.00 | 0.00 |
| ATOM | 2279 | CA | CYS | 1116 | 11.461 | 8.126 | 15.631 | 1.00 | 30.15 |
| ATOM | 2280 | CB | CYS | 1116 | 12.380 | 7.150 | 16.329 | 1.00 | 29.68 |
| ATOM | 2281 | SG | CYS | 1116 | 12.500 | 7.297 | 18.146 | 1.00 | 37.39 |
| ATOM | 2282 | C | CYS | 1116 | 11.610 | 7.918 | 14.122 | 1.00 | 32.08 |
| ATOM | 2283 | O | CYS | 1116 | 12.364 | 8.631 | 13.418 | 1.00 | 30.84 |
| ATOM | 2284 | N | ARG | 1117 | 10.901 | 6.905 | 13.573 | 1.00 | 34.49 |
| ATOM | 2285 | H | ARG | 1117 | 10.328 | 6.354 | 14.148 | 1.00 | 0.00 |
| ATOM | 2286 | CA | ARG | 1117 | 10.981 | 6.624 | 12.106 | 1.00 | 36.29 |
| ATOM | 2287 | CB | ARG | 1117 | 10.066 | 5.363 | 11.854 | 1.00 | 38.80 |
| ATOM | 2288 | CG | ARG | 1117 | 9.691 | 4.777 | 10.452 | 1.00 | 43.19 |
| ATOM | 2289 | CD | ARG | 1117 | 8.120 | 4.529 | 10.251 | 1.00 | 45.38 |
| ATOM | 2290 | NE | ARG | 1117 | 7.363 | 5.297 | 9.185 | 1.00 | 45.12 |
| ATOM | 2291 | HE | ARG | 1117 | 7.792 | 5.408 | 8.312 | 1.00 | 0.00 |
| ATOM | 2292 | CZ | ARG | 1117 | 6.123 | 5.868 | 9.341 | 1.00 | 49.21 |
| ATOM | 2293 | NH1 | ARG | 1117 | 5.494 | 6.533 | 8.327 | 1.00 | 49.58 |
| ATOM | 2294 | HH11 | ARG | 1117 | 5.922 | 6.621 | 7.427 | 1.00 | 0.00 |
| ATOM | 2295 | HH12 | ARG | 1117 | 4.590 | 6.932 | 8.483 | 1.00 | 0.00 |
| ATOM | 2296 | NH2 | ARG | 1117 | 5.484 | 5.838 | 10.529 | 1.00 | 49.83 |
| ATOM | 2297 | HH21 | ARG | 1117 | 5.927 | 5.412 | 11.316 | 1.00 | 0.00 |
| ATOM | 2298 | HH22 | ARG | 1117 | 4.574 | 6.241 | 10.621 | 1.00 | 0.00 |
| ATOM | 2299 | C | ARG | 1117 | 10.561 | 7.895 | 11.302 | 1.00 | 37.60 |
| ATOM | 2300 | O | ARG | 1117 | 11.305 | 8.411 | 10.435 | 1.00 | 38.84 |
| ATOM | 2301 | N | ARG | 1118 | 9.413 | 8.495 | 11.699 | 1.00 | 35.70 |
| ATOM | 2302 | H | ARG | 1118 | 8.958 | 8.100 | 12.478 | 1.00 | 0.00 |
| ATOM | 2303 | CA | ARG | 1118 | 8.805 | 9.724 | 11.090 | 1.00 | 33.46 |
| ATOM | 2304 | CB | ARG | 1118 | 7.534 | 10.061 | 11.886 | 1.00 | 36.16 |
| ATOM | 2305 | CG | ARG | 1118 | 6.285 | 9.621 | 11.144 | 1.00 | 40.88 |
| ATOM | 2306 | CD | ARG | 1118 | 5.245 | 9.085 | 12.133 | 1.00 | 49.93 |
| ATOM | 2307 | NE | ARG | 1118 | 4.917 | 10.065 | 13.197 | 1.00 | 56.22 |
| ATOM | 2308 | HE | ARG | 1118 | 4.839 | 11.011 | 12.951 | 1.00 | 0.00 |
| ATOM | 2309 | CZ | ARG | 1118 | 4.680 | 9.730 | 14.476 | 1.00 | 57.65 |
| ATOM | 2310 | NH1 | ARG | 1118 | 4.395 | 10.727 | 15.280 | 1.00 | 60.21 |
| ATOM | 2311 | HH11 | ARG | 1118 | 4.361 | 11.661 | 14.925 | 1.00 | 0.00 |
| ATOM | 2312 | HH12 | ARG | 1118 | 4.212 | 10.547 | 16.246 | 1.00 | 0.00 |
| ATOM | 2313 | NH2 | ARG | 1118 | 4.728 | 8.487 | 14.993 | 1.00 | 61.32 |
| ATOM | 2314 | HH21 | ARG | 1118 | 4.956 | 7.702 | 14.415 | 1.00 | 0.00 |
| ATOM | 2315 | HH22 | ARG | 1118 | 4.534 | 8.349 | 15.965 | 1.00 | 0.00 |
| ATOM | 2316 | C | ARG | 1118 | 9.743 | 10.936 | 11.046 | 1.00 | 30.71 |
| ATOM | 2317 | O | ARG | 1118 | 9.928 | 11.695 | 10.080 | 1.00 | 26.20 |
| ATOM | 2318 | N | LEU | 1119 | 10.347 | 11.121 | 12.185 | 1.00 | 29.43 |
| ATOM | 2319 | H | LEU | 1119 | 10.105 | 10.562 | 12.958 | 1.00 | 0.00 |
| ATOM | 2320 | CA | LEU | 1119 | 11.329 | 12.192 | 12.322 | 1.00 | 30.49 |
| ATOM | 2321 | CB | LEU | 1119 | 11.801 | 12.090 | 13.752 | 1.00 | 28.84 |
| ATOM | 2322 | CG | LEU | 1119 | 12.497 | 13.329 | 14.212 | 1.00 | 28.84 |
| ATOM | 2323 | CD1 | LEU | 1119 | 11.326 | 14.294 | 14.567 | 1.00 | 28.60 |
| ATOM | 2324 | CD2 | LEU | 1119 | 13.621 | 12.991 | 15.281 | 1.00 | 28.62 |
| ATOM | 2325 | C | LEU | 1119 | 12.493 | 12.002 | 11.287 | 1.00 | 30.90 |
| ATOM | 2326 | O | LEU | 1119 | 13.013 | 12.910 | 10.617 | 1.00 | 28.45 |
| ATOM | 2327 | N | LYS | 1120 | 13.030 | 10.784 | 11.231 | 1.00 | 33.33 |
| ATOM | 2328 | H | LYS | 1120 | 12.742 | 10.077 | 11.856 | 1.00 | 0.00 |
| ATOM | 2329 | CA | LYS | 1120 | 14.113 | 10.558 | 10.228 | 1.00 | 37.65 |
| ATOM | 2330 | CB | LYS | 1120 | 14.700 | 9.081 | 10.318 | 1.00 | 39.42 |
| ATOM | 2331 | CG | LYS | 1120 | 16.118 | 8.914 | 9.686 | 1.00 | 40.91 |
| ATOM | 2332 | CD | LYS | 1120 | 16.833 | 7.544 | 9.952 | 1.00 | 44.41 |
| ATOM | 2333 | CE | LYS | 1120 | 18.345 | 7.359 | 9.458 | 1.00 | 47.43 |
| ATOM | 2334 | NZ | LYS | 1120 | 19.393 | 7.208 | 10.526 | 1.00 | 48.22 |
| ATOM | 2335 | HZ1 | LYS | 1120 | 19.089 | 6.482 | 11.208 | 1.00 | 0.00 |
| ATOM | 2336 | HZ2 | LYS | 1120 | 19.544 | 8.103 | 11.033 | 1.00 | 0.00 |
| ATOM | 2337 | HZ3 | LYS | 1120 | 20.290 | 6.900 | 10.097 | 1.00 | 0.00 |
| ATOM | 2338 | C | LYS | 1120 | 13.554 | 10.810 | 8.806 | 1.00 | 36.20 |
| ATOM | 2339 | O | LYS | 1120 | 14.306 | 11.126 | 7.878 | 1.00 | 37.44 |
| ATOM | 2340 | N | GLU | 1121 | 12.245 | 10.601 | 8.587 | 1.00 | 36.63 |
| ATOM | 2341 | H | GLU | 1121 | 11.689 | 10.181 | 9.276 | 1.00 | 0.00 |
| ATOM | 2342 | CA | GLU | 1121 | 11.744 | 10.880 | 7.222 | 1.00 | 39.38 |
| ATOM | 2343 | CB | GLU | 1121 | 10.598 | 9.906 | 6.822 | 1.00 | 41.61 |
| ATOM | 2344 | CG | GLU | 1121 | 9.322 | 10.047 | 7.617 | 1.00 | 48.48 |
| ATOM | 2345 | CD | GLU | 1121 | 8.848 | 8.652 | 7.901 | 1.00 | 51.14 |
| ATOM | 2346 | OE1 | GLU | 1121 | 7.645 | 8.411 | 7.718 | 1.00 | 54.04 |
| ATOM | 2347 | OE2 | GLU | 1121 | 9.693 | 7.840 | 8.298 | 1.00 | 52.47 |
| ATOM | 2348 | C | GLU | 1121 | 11.282 | 12.305 | 6.912 | 1.00 | 37.69 |
| ATOM | 2349 | O | GLU | 1121 | 10.715 | 12.577 | 5.829 | 1.00 | 37.53 |
| ATOM | 2350 | N | GLY | 1122 | 11.582 | 13.244 | 7.855 | 1.00 | 36.25 |
| ATOM | 2351 | H | GLY | 1122 | 12.094 | 12.954 | 8.640 | 1.00 | 0.00 |
| ATOM | 2352 | CA | GLY | 1122 | 11.242 | 14.688 | 7.657 | 1.00 | 31.49 |
| ATOM | 2353 | C | GLY | 1122 | 10.130 | 15.294 | 8.464 | 1.00 | 27.70 |
| ATOM | 2354 | O | GLY | 1122 | 9.837 | 16.467 | 8.332 | 1.00 | 25.99 |
| ATOM | 2355 | N | THR | 1123 | 9.415 | 14.487 | 9.202 | 1.00 | 28.69 |
| ATOM | 2356 | H | THR | 1123 | 9.607 | 13.522 | 9.162 | 1.00 | 0.00 |
| ATOM | 2357 | CA | THR | 1123 | 8.347 | 15.021 | 10.064 | 1.00 | 31.94 |
| ATOM | 2358 | CB | THR | 1123 | 7.525 | 13.879 | 10.788 | 1.00 | 33.61 |
| ATOM | 2359 | OG1 | THR | 1123 | 7.031 | 12.947 | 9.809 | 1.00 | 37.66 |
| ATOM | 2360 | HG1 | THR | 1123 | 6.569 | 12.226 | 10.250 | 1.00 | 0.00 |
| ATOM | 2361 | CG2 | THR | 1123 | 6.305 | 14.463 | 11.552 | 1.00 | 33.08 |
| ATOM | 2362 | C | THR | 1123 | 8.961 | 15.952 | 11.160 | 1.00 | 31.41 |
| ATOM | 2363 | O | THR | 1123 | 9.875 | 15.626 | 11.938 | 1.00 | 30.58 |
| ATOM | 2364 | N | ARG | 1124 | 8.345 | 17.140 | 11.271 | 1.00 | 28.62 |
| ATOM | 2365 | H | ARG | 1124 | 7.554 | 17.238 | 10.708 | 1.00 | 0.00 |
| ATOM | 2366 | CA | ARG | 1124 | 8.712 | 18.247 | 12.192 | 1.00 | 24.77 |
| ATOM | 2367 | CB | ARG | 1124 | 9.431 | 19.297 | 11.390 | 1.00 | 20.62 |
| ATOM | 2368 | CG | ARG | 1124 | 10.603 | 18.777 | 10.618 | 1.00 | 20.53 |
| ATOM | 2369 | CD | ARG | 1124 | 11.627 | 18.220 | 11.581 | 1.00 | 18.03 |
| ATOM | 2370 | NE | ARG | 1124 | 12.860 | 17.909 | 10.897 | 1.00 | 18.83 |
| ATOM | 2371 | HE | ARG | 1124 | 13.485 | 18.641 | 10.738 | 1.00 | 0.00 |
| ATOM | 2372 | CZ | ARG | 1124 | 13.150 | 16.714 | 10.441 | 1.00 | 16.63 |
| ATOM | 2373 | NH1 | ARG | 1124 | 12.347 | 15.674 | 10.519 | 1.00 | 18.66 |
| ATOM | 2374 | HH11 | ARG | 1124 | 11.457 | 15.795 | 10.945 | 1.00 | 0.00 |
| ATOM | 2375 | HH12 | ARG | 1124 | 12.646 | 14.803 | 10.151 | 1.00 | 0.00 |
| ATOM | 2376 | NH2 | ARG | 1124 | 14.381 | 16.521 | 10.009 | 1.00 | 17.03 |
| ATOM | 2377 | HH21 | ARG | 1124 | 15.040 | 17.264 | 10.039 | 1.00 | 0.00 |
| ATOM | 2378 | HH22 | ARG | 1124 | 14.672 | 15.628 | 9.637 | 1.00 | 0.00 |
| ATOM | 2379 | C | ARG | 1124 | 7.512 | 18.926 | 12.877 | 1.00 | 22.00 |
| ATOM | 2380 | O | ARG | 1124 | 6.418 | 18.935 | 12.334 | 1.00 | 23.78 |
| ATOM | 2381 | N | MET | 1125 | 7.649 | 19.468 | 14.053 | 1.00 | 17.13 |
| ATOM | 2382 | H | MET | 1125 | 8.461 | 19.285 | 14.564 | 1.00 | 0.00 |
| ATOM | 2383 | CA | MET | 1125 | 6.571 | 20.190 | 14.705 | 1.00 | 14.36 |
| ATOM | 2384 | CB | MET | 1125 | 6.987 | 20.833 | 16.021 | 1.00 | 16.33 |
| ATOM | 2385 | CG | MET | 1125 | 6.945 | 19.829 | 17.160 | 1.00 | 16.47 |
| ATOM | 2386 | SD | MET | 1125 | 7.490 | 20.440 | 18.739 | 1.00 | 16.74 |
| ATOM | 2387 | CE | MET | 1125 | 9.307 | 20.415 | 18.668 | 1.00 | 11.39 |
| ATOM | 2388 | C | MET | 1125 | 6.113 | 21.354 | 13.857 | 1.00 | 15.64 |
| ATOM | 2389 | O | MET | 1125 | 6.773 | 21.897 | 12.993 | 1.00 | 13.43 |
| ATOM | 2390 | N | ARG | 1126 | 4.848 | 21.689 | 14.124 | 1.00 | 16.28 |
| ATOM | 2391 | H | ARG | 1126 | 4.349 | 21.120 | 14.737 | 1.00 | 0.00 |
| ATOM | 2392 | CA | ARG | 1126 | 4.212 | 22.883 | 13.487 | 1.00 | 19.97 |
| ATOM | 2393 | CB | ARG | 1126 | 2.672 | 22.685 | 13.438 | 1.00 | 17.23 |
| ATOM | 2394 | C | ARG | 1126 | 4.600 | 24.125 | 14.354 | 1.00 | 18.69 |
| ATOM | 2395 | O | ARG | 1126 | 5.285 | 24.055 | 15.379 | 1.00 | 20.62 |
| ATOM | 2396 | N | ALA | 1127 | 4.282 | 25.301 | 13.861 | 1.00 | 19.38 |
| ATOM | 2397 | H | ALA | 1127 | 3.815 | 25.302 | 12.994 | 1.00 | 0.00 |
| ATOM | 2398 | CA | ALA | 1127 | 4.602 | 26.573 | 14.514 | 1.00 | 18.43 |
| ATOM | 2399 | CB | ALA | 1127 | 4.093 | 27.797 | 13.727 | 1.00 | 13.49 |
| ATOM | 2400 | C | ALA | 1127 | 3.998 | 26.672 | 15.876 | 1.00 | 18.55 |
| ATOM | 2401 | O | ALA | 1127 | 2.778 | 26.516 | 16.034 | 1.00 | 21.08 |
| ATOM | 2402 | N | PRO | 1128 | 4.848 | 27.020 | 16.856 | 1.00 | 15.18 |
| ATOM | 2403 | CD | PRO | 1128 | 6.292 | 27.255 | 16.827 | 1.00 | 10.83 |
| ATOM | 2404 | CA | PRO | 1128 | 4.263 | 27.356 | 18.126 | 1.00 | 14.56 |
| ATOM | 2405 | CB | PRO | 1128 | 5.565 | 27.512 | 18.974 | 1.00 | 10.81 |
| ATOM | 2406 | CG | PRO | 1128 | 6.563 | 28.092 | 18.086 | 1.00 | 7.63 |
| ATOM | 2407 | C | PRO | 1128 | 3.284 | 28.577 | 18.024 | 1.00 | 14.54 |
| ATOM | 2408 | O | PRO | 1128 | 3.289 | 29.421 | 17.140 | 1.00 | 12.35 |
| ATOM | 2409 | N | ASP | 1129 | 2.415 | 28.756 | 18.986 | 1.00 | 16.63 |
| ATOM | 2410 | H | ASP | 1129 | 2.413 | 28.039 | 19.657 | 1.00 | 0.00 |
| ATOM | 2411 | CA | ASP | 1129 | 1.433 | 29.908 | 19.006 | 1.00 | 19.14 |
| ATOM | 2412 | CB | ASP | 1129 | 0.448 | 29.801 | 20.265 | 1.00 | 16.34 |
| ATOM | 2413 | CG | ASP | 1129 | −0.561 | 28.677 | 20.145 | 1.00 | 18.57 |
| ATOM | 2414 | OD1 | ASP | 1129 | −1.198 | 28.320 | 21.135 | 1.00 | 19.19 |
| ATOM | 2415 | OD2 | ASP | 1129 | −0.733 | 28.152 | 19.046 | 1.00 | 24.04 |
| ATOM | 2416 | C | ASP | 1129 | 1.967 | 31.351 | 19.005 | 1.00 | 18.36 |
| ATOM | 2417 | O | ASP | 1129 | 1.315 | 32.270 | 18.549 | 1.00 | 17.16 |
| ATOM | 2418 | N | TYR | 1130 | 3.145 | 31.583 | 19.552 | 1.00 | 18.26 |
| ATOM | 2419 | H | TYR | 1130 | 3.647 | 30.827 | 19.927 | 1.00 | 0.00 |
| ATOM | 2420 | CA | TYR | 1130 | 3.718 | 32.937 | 19.706 | 1.00 | 16.09 |
| ATOM | 2421 | CB | TYR | 1130 | 4.085 | 33.032 | 21.223 | 1.00 | 13.96 |
| ATOM | 2422 | CG | TYR | 1130 | 3.035 | 32.500 | 22.132 | 1.00 | 12.83 |
| ATOM | 2423 | CD1 | TYR | 1130 | 3.092 | 31.207 | 22.727 | 1.00 | 12.72 |
| ATOM | 2424 | CE1 | TYR | 1130 | 2.050 | 30.782 | 23.596 | 1.00 | 11.93 |
| ATOM | 2425 | CD2 | TYR | 1130 | 1.950 | 33.354 | 22.401 | 1.00 | 11.56 |
| ATOM | 2426 | CE2 | TYR | 1130 | 0.934 | 32.937 | 23.244 | 1.00 | 12.83 |
| ATOM | 2427 | CZ | TYR | 1130 | 0.989 | 31.671 | 23.843 | 1.00 | 11.75 |
| ATOM | 2428 | OH | TYR | 1130 | −0.082 | 31.326 | 24.631 | 1.00 | 15.40 |
| ATOM | 2429 | HH | TYR | 1130 | −0.626 | 32.107 | 24.784 | 1.00 | 0.00 |
| ATOM | 2430 | C | TYR | 1130 | 4.920 | 33.226 | 18.796 | 1.00 | 16.57 |
| ATOM | 2431 | O | TYR | 1130 | 5.683 | 34.184 | 18.914 | 1.00 | 16.17 |
| ATOM | 2432 | N | TER | 1131 | 5.215 | 32.237 | 17.993 | 1.00 | 15.58 |
| ATOM | 2433 | H | THR | 1131 | 4.648 | 31.444 | 18.029 | 1.00 | 0.00 |
| ATOM | 2434 | CA | TER | 1131 | 6.315 | 32.314 | 17.062 | 1.00 | 16.07 |
| ATOM | 2435 | CB | THR | 1131 | 6.464 | 30.985 | 16.248 | 1.00 | 17.09 |
| ATOM | 2436 | OG1 | THR | 1131 | 7.716 | 31.079 | 15.634 | 1.00 | 19.72 |
| ATOM | 2437 | HG1 | THR | 1131 | 7.911 | 30.258 | 15.178 | 1.00 | 0.00 |
| ATOM | 2438 | CG2 | THR | 1131 | 5.528 | 30.771 | 14.988 | 1.00 | 16.73 |
| ATOM | 2439 | C | THR | 1131 | 6.174 | 33.402 | 16.037 | 1.00 | 19.30 |
| ATOM | 2440 | O | THR | 1131 | 5.088 | 33.885 | 15.736 | 1.00 | 19.44 |
| ATOM | 2441 | N | THR | 1132 | 7.288 | 33.830 | 15.480 | 1.00 | 20.53 |
| ATOM | 2442 | H | THR | 1132 | 8.121 | 33.450 | 15.817 | 1.00 | 0.00 |
| ATOM | 2443 | CA | THR | 1132 | 7.264 | 34.779 | 14.339 | 1.00 | 21.31 |
| ATOM | 2444 | CB | THR | 1132 | 8.379 | 35.785 | 14.304 | 1.00 | 21.97 |
| ATOM | 2445 | OG1 | THR | 1132 | 9.696 | 35.100 | 14.335 | 1.00 | 21.19 |
| ATOM | 2446 | HG1 | THR | 1132 | 10.309 | 35.532 | 14.954 | 1.00 | 0.00 |
| ATOM | 2447 | CG2 | THR | 1132 | 8.053 | 36.841 | 15.405 | 1.00 | 21.23 |
| ATOM | 2448 | C | THR | 1132 | 7.534 | 33.918 | 13.132 | 1.00 | 21.31 |
| ATOM | 2449 | O | THR | 1132 | 8.161 | 32.868 | 13.264 | 1.00 | 23.62 |
| ATOM | 2450 | N | PRO | 1133 | 7.152 | 34.274 | 11.910 | 1.00 | 21.52 |
| ATOM | 2451 | CD | PRO | 1133 | 6.347 | 35.465 | 11.451 | 1.00 | 23.15 |
| ATOM | 2452 | CA | PRO | 1133 | 7.537 | 33.434 | 10.759 | 1.00 | 20.55 |
| ATOM | 2453 | CB | PRO | 1133 | 7.086 | 34.228 | 9.499 | 1.00 | 21.59 |
| ATOM | 2454 | CG | PRO | 1133 | 5.882 | 35.016 | 10.061 | 1.00 | 23.03 |
| ATOM | 2455 | C | PRO | 1133 | 8.999 | 33.098 | 10.714 | 1.00 | 18.40 |
| ATOM | 2456 | O | PRO | 1133 | 9.392 | 31.973 | 10.495 | 1.00 | 17.07 |
| ATOM | 2457 | N | GLU | 1134 | 9.871 | 34.072 | 10.931 | 1.00 | 19.10 |
| ATOM | 2458 | H | GLU | 1134 | 9.498 | 34.953 | 11.098 | 1.00 | 0.00 |
| ATOM | 2459 | CA | GLU | 1134 | 11.315 | 33.793 | 10.844 | 1.00 | 21.85 |
| ATOM | 2460 | CB | GLU | 1134 | 12.149 | 35.088 | 10.756 | 1.00 | 24.04 |
| ATOM | 2461 | CG | GLU | 1134 | 11.530 | 36.344 | 11.332 | 1.00 | 32.68 |
| ATOM | 2462 | CD | GLU | 1134 | 10.356 | 37.064 | 10.671 | 1.00 | 34.12 |
| ATOM | 2463 | OE1 | GLU | 1134 | 9.439 | 37.468 | 11.379 | 1.00 | 38.75 |
| ATOM | 2464 | OE2 | GLU | 1134 | 10.385 | 37.332 | 9.475 | 1.00 | 40.37 |
| ATOM | 2465 | C | GLU | 1134 | 11.827 | 32.947 | 11.934 | 1.00 | 19.77 |
| ATOM | 2466 | O | GLU | 1134 | 12.782 | 32.186 | 11.743 | 1.00 | 18.37 |
| ATOM | 2467 | N | MET | 1135 | 11.179 | 33.059 | 13.086 | 1.00 | 20.19 |
| ATOM | 2468 | H | MET | 1135 | 10.433 | 33.680 | 13.197 | 1.00 | 0.00 |
| ATOM | 2469 | CA | MET | 1135 | 11.573 | 32.181 | 14.175 | 1.00 | 18.62 |
| ATOM | 2470 | CB | MET | 1135 | 10.917 | 32.613 | 15.511 | 1.00 | 18.20 |
| ATOM | 2471 | CG | MET | 1135 | 11.672 | 33.780 | 16.264 | 1.00 | 24.38 |
| ATOM | 2472 | SD | MET | 1135 | 13.511 | 33.684 | 16.615 | 1.00 | 27.46 |
| ATOM | 2473 | CE | MET | 1135 | 13.700 | 31.959 | 16.776 | 1.00 | 21.30 |
| ATOM | 2474 | C | MET | 1135 | 11.150 | 30.728 | 13.809 | 1.00 | 18.52 |
| ATOM | 2475 | O | MET | 1135 | 11.906 | 29.768 | 13.996 | 1.00 | 20.46 |
| ATOM | 2476 | N | TYR | 1136 | 9.997 | 30.481 | 13.181 | 1.00 | 16.89 |
| ATOM | 2477 | H | TYR | 1136 | 9.401 | 31.243 | 13.011 | 1.00 | 0.00 |
| ATOM | 2478 | CA | TYR | 1136 | 9.612 | 29.118 | 12.842 | 1.00 | 13.96 |
| ATOM | 2479 | CB | TYR | 1136 | 8.151 | 29.167 | 12.364 | 1.00 | 15.09 |
| ATOM | 2480 | CG | TYR | 1136 | 7.771 | 27.750 | 12.199 | 1.00 | 18.32 |
| ATOM | 2481 | CD1 | TYR | 1136 | 7.999 | 26.737 | 13.164 | 1.00 | 14.02 |
| ATOM | 2482 | CE1 | TYR | 1136 | 7.648 | 25.400 | 12.902 | 1.00 | 15.33 |
| ATOM | 2483 | CD2 | TYR | 1136 | 7.174 | 27.435 | 10.971 | 1.00 | 19.19 |
| ATOM | 2484 | CE2 | TYR | 1136 | 6.811 | 26.089 | 10.699 | 1.00 | 16.36 |
| ATOM | 2485 | CZ | TYR | 1136 | 7.056 | 25.094 | 11.664 | 1.00 | 14.57 |
| ATOM | 2486 | OH | TYR | 1136 | 6.678 | 23.828 | 11.375 | 1.00 | 17.48 |
| ATOM | 2487 | HH | TYR | 1136 | 6.918 | 23.367 | 12.165 | 1.00 | 0.00 |
| ATOM | 2488 | C | TYR | 1136 | 10.547 | 28.522 | 11.807 | 1.00 | 15.81 |
| ATOM | 2489 | O | TYR | 1136 | 10.945 | 27.361 | 11.766 | 1.00 | 15.10 |
| ATOM | 2490 | N | GLN | 1137 | 10.915 | 29.325 | 10.866 | 1.00 | 16.99 |
| ATOM | 2491 | H | GLN | 1137 | 10.549 | 30.233 | 10.854 | 1.00 | 0.00 |
| ATOM | 2492 | CA | GLN | 1137 | 11.829 | 28.859 | 9.840 | 1.00 | 16.88 |
| ATOM | 2493 | CB | GLN | 1137 | 11.937 | 29.943 | 8.790 | 1.00 | 19.46 |
| ATOM | 2494 | CG | GLN | 1137 | 12.920 | 29.407 | 7.670 | 1.00 | 23.56 |
| ATOM | 2495 | CD | GLN | 1137 | 12.417 | 28.138 | 7.007 | 1.00 | 22.02 |
| ATOM | 2496 | OE1 | GLN | 1137 | 11.309 | 28.168 | 6.474 | 1.00 | 24.56 |
| ATOM | 2497 | NE2 | GLN | 1137 | 13.170 | 27.033 | 7.030 | 1.00 | 19.32 |
| ATOM | 2498 | HE21 | GLN | 1137 | 14.039 | 27.019 | 7.461 | 1.00 | 0.00 |
| ATOM | 2499 | HE22 | GLN | 1137 | 12.819 | 26.230 | 6.588 | 1.00 | 0.00 |
| ATOM | 2500 | C | GLN | 1137 | 13.187 | 28.545 | 10.455 | 1.00 | 15.26 |
| ATOM | 2501 | O | GLN | 1137 | 13.966 | 27.728 | 9.996 | 1.00 | 12.87 |
| ATOM | 2502 | N | THR | 1138 | 13.577 | 29.300 | 11.483 | 1.00 | 15.59 |
| ATOM | 2503 | H | THR | 1138 | 13.094 | 30.123 | 11.711 | 1.00 | 0.00 |
| ATOM | 2504 | CA | THR | 1138 | 14.869 | 29.008 | 12.105 | 1.00 | 15.31 |
| ATOM | 2505 | CB | THR | 1138 | 15.221 | 30.148 | 13.089 | 1.00 | 15.17 |
| ATOM | 2506 | OG1 | THR | 1138 | 15.494 | 31.349 | 12.338 | 1.00 | 15.62 |
| ATOM | 2507 | HG1 | THR | 1138 | 15.605 | 32.077 | 12.951 | 1.00 | 0.00 |
| ATOM | 2508 | CG2 | THR | 1138 | 16.450 | 29.834 | 13.907 | 1.00 | 12.25 |
| ATOM | 2509 | C | THR | 1138 | 14.787 | 27.630 | 12.779 | 1.00 | 17.14 |
| ATOM | 2510 | O | THR | 1138 | 15.699 | 26.814 | 12.665 | 1.00 | 21.42 |
| ATOM | 2511 | N | MET | 1139 | 13.703 | 27.308 | 13.461 | 1.00 | 15.38 |
| ATOM | 2512 | H | MET | 1139 | 13.025 | 28.001 | 13.574 | 1.00 | 0.00 |
| ATOM | 2513 | CA | MET | 1139 | 13.512 | 25.984 | 14.077 | 1.00 | 14.79 |
| ATOM | 2514 | CB | MET | 1139 | 12.120 | 25.909 | 14.649 | 1.00 | 14.35 |
| ATOM | 2515 | CG | MET | 1139 | 11.830 | 26.904 | 15.784 | 1.00 | 10.89 |
| ATOM | 2516 | SD | MET | 1139 | 10.132 | 26.836 | 16.364 | 1.00 | 14.97 |
| ATOM | 2517 | CE | MET | 1139 | 10.067 | 28.530 | 17.053 | 1.00 | 7.31 |
| ATOM | 2518 | C | MET | 1139 | 13.662 | 24.930 | 12.969 | 1.00 | 15.20 |
| ATOM | 2519 | O | MET | 1139 | 14.434 | 23.984 | 13.095 | 1.00 | 16.89 |
| ATOM | 2520 | N | LEU | 1140 | 12.957 | 25.043 | 11.837 | 1.00 | 17.38 |
| ATOM | 2521 | H | LEU | 1140 | 12.285 | 25.753 | 11.786 | 1.00 | 0.00 |
| ATOM | 2522 | CA | LEU | 1140 | 13.149 | 24.083 | 10.707 | 1.00 | 15.15 |
| ATOM | 2523 | CB | LEU | 1140 | 12.197 | 24.464 | 9.573 | 1.00 | 16.23 |
| ATOM | 2524 | CG | LEU | 1140 | 10.648 | 24.534 | 9.867 | 1.00 | 18.39 |
| ATOM | 2525 | CD1 | LEU | 1140 | 9.881 | 25.126 | 8.622 | 1.00 | 16.89 |
| ATOM | 2526 | CD2 | LEU | 1140 | 10.112 | 23.128 | 10.164 | 1.00 | 17.26 |
| ATOM | 2527 | C | LEU | 1140 | 14.631 | 24.081 | 10.201 | 1.00 | 16.04 |
| ATOM | 2528 | O | LEU | 1140 | 15.174 | 23.037 | 9.859 | 1.00 | 16.04 |
| ATOM | 2529 | N | ASP | 1141 | 15.374 | 25.195 | 10.142 | 1.00 | 15.59 |
| ATOM | 2530 | H | ASP | 1141 | 14.946 | 26.042 | 10.387 | 1.00 | 0.00 |
| ATOM | 2531 | CA | ASP | 1141 | 16.780 | 25.139 | 9.717 | 1.00 | 15.51 |
| ATOM | 2532 | CB | ASP | 1141 | 17.370 | 26.538 | 9.765 | 1.00 | 16.33 |
| ATOM | 2533 | CG | ASP | 1141 | 16.756 | 27.475 | 8.750 | 1.00 | 18.70 |
| ATOM | 2534 | OD1 | ASP | 1141 | 16.959 | 28.656 | 8.885 | 1.00 | 19.18 |
| ATOM | 2535 | OD2 | ASP | 1141 | 16.086 | 27.067 | 7.819 | 1.00 | 22.21 |
| ATOM | 2536 | C | ASP | 1141 | 17.585 | 24.232 | 10.664 | 1.00 | 19.01 |
| ATOM | 2537 | O | ASP | 1141 | 18.410 | 23.353 | 10.364 | 1.00 | 20.85 |
| ATOM | 2538 | N | CYS | 1142 | 17.373 | 24.495 | 11.949 | 1.00 | 19.78 |
| ATOM | 2539 | H | CYS | 1142 | 16.741 | 25.213 | 12.169 | 1.00 | 0.00 |
| ATOM | 2540 | CA | CYS | 1142 | 18.033 | 23.745 | 13.048 | 1.00 | 18.18 |
| ATOM | 2541 | CB | CYS | 1142 | 17.579 | 24.296 | 14.452 | 1.00 | 20.47 |
| ATOM | 2542 | SG | CYS | 1142 | 18.112 | 25.988 | 14.909 | 1.00 | 15.64 |
| ATOM | 2543 | C | CYS | 1142 | 17.634 | 22.276 | 12.952 | 1.00 | 17.82 |
| ATOM | 2544 | O | CYS | 1142 | 18.373 | 21.373 | 13.353 | 1.00 | 13.88 |
| ATOM | 2545 | N | TRP | 1143 | 16.386 | 22.013 | 12.529 | 1.00 | 17.39 |
| ATOM | 2546 | H | TRP | 1143 | 15.739 | 22.725 | 12.344 | 1.00 | 0.00 |
| ATOM | 2547 | CA | TRP | 1143 | 16.050 | 20.593 | 12.395 | 1.00 | 19.18 |
| ATOM | 2548 | CB | TRP | 1143 | 14.592 | 20.407 | 12.789 | 1.00 | 17.63 |
| ATOM | 2549 | CG | TRP | 1143 | 14.251 | 20.973 | 14.129 | 1.00 | 19.30 |
| ATOM | 2550 | CD2 | TRP | 1143 | 12.961 | 21.497 | 14.459 | 1.00 | 18.44 |
| ATOM | 2551 | CE2 | TRP | 1143 | 12.958 | 21.639 | 15.899 | 1.00 | 17.01 |
| ATOM | 2552 | CE3 | TRP | 1143 | 11.792 | 21.806 | 13.677 | 1.00 | 15.52 |
| ATOM | 2553 | CD1 | TRP | 1143 | 14.959 | 20.870 | 15.350 | 1.00 | 14.80 |
| ATOM | 2554 | NE1 | TRP | 1143 | 14.208 | 21.238 | 16.403 | 1.00 | 16.74 |
| ATOM | 2555 | HE1 | TRP | 1143 | 14.498 | 21.159 | 17.337 | 1.00 | 0.00 |
| ATOM | 2556 | CZ2 | TRP | 1143 | 11.743 | 22.143 | 16.497 | 1.00 | 15.27 |
| ATOM | 2557 | CZ3 | TRP | 1143 | 10.622 | 22.321 | 14.295 | 1.00 | 12.55 |
| ATOM | 2558 | CH2 | TRP | 1143 | 10.607 | 22.457 | 15.698 | 1.00 | 14.07 |
| ATOM | 2559 | C | TRP | 1143 | 16.338 | 19.947 | 10.975 | 1.00 | 22.67 |
| ATOM | 2560 | O | TRP | 1143 | 15.739 | 18.941 | 10.540 | 1.00 | 25.77 |
| ATOM | 2561 | N | HIS | 1144 | 17.281 | 20.528 | 10.210 | 1.00 | 20.00 |
| ATOM | 2562 | H | HIS | 1144 | 17.709 | 21.330 | 10.559 | 1.00 | 0.00 |
| ATOM | 2563 | CA | HIS | 1144 | 17.676 | 19.959 | 8.935 | 1.00 | 19.52 |
| ATOM | 2564 | C | HIS | 1144 | 18.098 | 18.435 | 9.111 | 1.00 | 22.18 |
| ATOM | 2565 | O | HIS | 1144 | 18.842 | 18.134 | 10.041 | 1.00 | 21.16 |
| ATOM | 2566 | CB | HIS | 1144 | 18.844 | 20.815 | 8.455 | 1.00 | 18.01 |
| ATOM | 2567 | CG | HIS | 1144 | 19.080 | 20.424 | 7.112 | 1.00 | 18.66 |
| ATOM | 2568 | ND1 | HIS | 1144 | 18.648 | 21.053 | 6.030 | 1.00 | 20.15 |
| ATOM | 2569 | HD1 | HIS | 1144 | 18.291 | 21.960 | 5.984 | 1.00 | 0.00 |
| ATOM | 2570 | CD2 | HIS | 1144 | 19.591 | 19.235 | 6.711 | 1.00 | 18.51 |
| ATOM | 2571 | NE2 | HIS | 1144 | 19.463 | 19.130 | 5.412 | 1.00 | 21.04 |
| ATOM | 2572 | CE1 | HIS | 1144 | 18.875 | 20.248 | 4.962 | 1.00 | 19.84 |
| ATOM | 2573 | N | GLY | 1145 | 17.632 | 17.435 | 8.302 | 1.00 | 21.94 |
| ATOM | 2574 | H | GLY | 1145 | 16.877 | 17.721 | 7.738 | 1.00 | 0.00 |
| ATOM | 2575 | CA | GLY | 1145 | 18.033 | 16.018 | 8.378 | 1.00 | 21.04 |
| ATOM | 2576 | C | GLY | 1145 | 19.570 | 15.892 | 8.503 | 1.00 | 23.68 |
| ATOM | 2577 | O | GLY | 1145 | 20.084 | 15.045 | 9.227 | 1.00 | 24.29 |
| ATOM | 2578 | N | GLU | 1146 | 20.338 | 16.685 | 7.713 | 1.00 | 26.59 |
| ATOM | 2579 | H | GLU | 1146 | 19.820 | 17.062 | 6.984 | 1.00 | 0.00 |
| ATOM | 2580 | CA | GLU | 1146 | 21.835 | 16.781 | 7.667 | 1.00 | 27.26 |
| ATOM | 2581 | CB | GLU | 1146 | 22.358 | 17.306 | 6.335 | 1.00 | 32.00 |
| ATOM | 2582 | CG | GLU | 1146 | 22.760 | 16.284 | 5.331 | 1.00 | 43.96 |
| ATOM | 2583 | CD | GLU | 1146 | 21.507 | 15.960 | 4.541 | 1.00 | 54.90 |
| ATOM | 2584 | OE1 | GLU | 1146 | 20.605 | 15.286 | 5.097 | 1.00 | 58.81 |
| ATOM | 2585 | OE2 | GLU | 1146 | 21.449 | 16.396 | 3.365 | 1.00 | 59.51 |
| ATOM | 2586 | C | GLU | 1146 | 22.595 | 17.655 | 8.692 | 1.00 | 23.69 |
| ATOM | 2587 | O | GLU | 1146 | 22.658 | 18.871 | 8.553 | 1.00 | 23.40 |
| ATOM | 2588 | N | PRO | 1147 | 23.327 | 17.073 | 9.633 | 1.00 | 21.25 |
| ATOM | 2589 | CD | PRO | 1147 | 23.535 | 15.631 | 9.841 | 1.00 | 18.71 |
| ATOM | 2590 | CA | PRO | 1147 | 23.920 | 17.836 | 10.711 | 1.00 | 20.73 |
| ATOM | 2591 | CB | PRO | 1147 | 24.826 | 16.825 | 11.426 | 1.00 | 17.05 |
| ATOM | 2592 | CG | PRO | 1147 | 24.023 | 15.566 | 11.274 | 1.00 | 16.94 |
| ATOM | 2593 | C | PRO | 1147 | 24.642 | 19.050 | 10.206 | 1.00 | 22.74 |
| ATOM | 2594 | O | PRO | 1147 | 24.542 | 20.206 | 10.659 | 1.00 | 20.77 |
| ATOM | 2595 | N | SER | 1148 | 25.420 | 18.728 | 9.173 | 1.00 | 25.97 |
| ATOM | 2596 | H | SER | 1148 | 25.384 | 17.798 | 8.860 | 1.00 | 0.00 |
| ATOM | 2597 | CA | SER | 1148 | 26.284 | 19.727 | 8.499 | 1.00 | 26.35 |
| ATOM | 2598 | CB | SER | 1148 | 27.237 | 19.080 | 7.486 | 1.00 | 26.07 |
| ATOM | 2599 | OG | SER | 1148 | 26.463 | 18.696 | 6.339 | 1.00 | 36.27 |
| ATOM | 2600 | HG | SER | 1148 | 27.026 | 18.188 | 5.739 | 1.00 | 0.00 |
| ATOM | 2601 | C | SER | 1148 | 25.476 | 20.780 | 7.781 | 1.00 | 25.01 |
| ATOM | 2602 | O | SER | 1148 | 26.002 | 21.847 | 7.508 | 1.00 | 25.74 |
| ATOM | 2603 | N | GLN | 1149 | 24.236 | 20.512 | 7.400 | 1.00 | 24.25 |
| ATOM | 2604 | H | GLN | 1149 | 23.949 | 19.586 | 7.428 | 1.00 | 0.00 |
| ATOM | 2605 | CA | GLN | 1149 | 23.428 | 21.550 | 6.750 | 1.00 | 24.40 |
| ATOM | 2606 | CB | GLN | 1149 | 22.347 | 21.001 | 5.847 | 1.00 | 27.07 |
| ATOM | 2607 | CG | GLN | 1149 | 22.982 | 20.231 | 4.707 | 1.00 | 32.73 |
| ATOM | 2608 | CD | GLN | 1149 | 23.399 | 21.257 | 3.741 | 1.00 | 33.98 |
| ATOM | 2609 | OE1 | GLN | 1149 | 22.680 | 21.643 | 2.808 | 1.00 | 33.42 |
| ATOM | 2610 | NE2 | GLN | 1149 | 24.611 | 21.732 | 3.958 | 1.00 | 36.71 |
| ATOM | 2611 | HE21 | GLN | 1149 | 25.183 | 21.440 | 4.683 | 1.00 | 0.00 |
| ATOM | 2612 | HE22 | GLN | 1149 | 24.883 | 22.423 | 3.317 | 1.00 | 0.00 |
| ATOM | 2613 | C | GLN | 1149 | 22.717 | 22.355 | 7.775 | 1.00 | 24.19 |
| ATOM | 2614 | O | GLN | 1149 | 21.964 | 23.244 | 7.381 | 1.00 | 24.66 |
| ATOM | 2615 | N | ARG | 1150 | 22.742 | 21.956 | 9.054 | 1.00 | 23.60 |
| ATOM | 2616 | H | ARG | 1150 | 23.236 | 21.155 | 9.321 | 1.00 | 0.00 |
| ATOM | 2617 | CA | ARG | 1150 | 22.057 | 22.799 | 10.082 | 1.00 | 21.42 |
| ATOM | 2618 | CB | ARG | 1150 | 21.872 | 22.053 | 11.427 | 1.00 | 21.61 |
| ATOM | 2619 | CG | ARG | 1150 | 20.747 | 21.033 | 11.485 | 1.00 | 21.83 |
| ATOM | 2620 | CD | ARG | 1150 | 20.969 | 20.044 | 12.643 | 1.00 | 18.56 |
| ATOM | 2621 | NE | ARG | 1150 | 20.339 | 18.769 | 12.268 | 1.00 | 17.92 |
| ATOM | 2622 | HE | ARG | 1150 | 19.572 | 18.793 | 11.668 | 1.00 | 0.00 |
| ATOM | 2623 | CZ | ARG | 1150 | 20.659 | 17.606 | 12.845 | 1.00 | 16.10 |
| ATOM | 2624 | NH1 | ARG | 1150 | 21.557 | 17.580 | 13.841 | 1.00 | 16.06 |
| ATOM | 2625 | HH11 | ARG | 1150 | 21.995 | 18.419 | 14.151 | 1.00 | 0.00 |
| ATOM | 2626 | HH12 | ARG | 1150 | 21.775 | 16.716 | 14.277 | 1.00 | 0.00 |
| ATOM | 2627 | NH2 | ARG | 1150 | 20.213 | 16.463 | 12.285 | 1.00 | 13.42 |
| ATOM | 2628 | HH21 | ARG | 1150 | 19.665 | 16.487 | 11.452 | 1.00 | 0.00 |
| ATOM | 2629 | HH22 | ARG | 1150 | 20.438 | 15.590 | 12.707 | 1.00 | 0.00 |
| ATOM | 2630 | C | ARG | 1150 | 22.969 | 24.026 | 10.348 | 1.00 | 19.08 |
| ATOM | 2631 | O | ARG | 1150 | 24.190 | 24.043 | 10.093 | 1.00 | 17.27 |
| ATOM | 2632 | N | PRO | 1151 | 22.372 | 25.146 | 10.810 | 1.00 | 17.32 |
| ATOM | 2633 | CD | PRO | 1151 | 20.945 | 25.466 | 10.797 | 1.00 | 12.13 |
| ATOM | 2634 | CA | PRO | 1151 | 23.196 | 26.297 | 11.214 | 1.00 | 16.48 |
| ATOM | 2635 | CB | PRO | 1151 | 22.212 | 27.450 | 11.579 | 1.00 | 14.64 |
| ATOM | 2636 | CG | PRO | 1151 | 20.880 | 26.650 | 11.774 | 1.00 | 11.90 |
| ATOM | 2637 | C | PRO | 1151 | 24.108 | 26.004 | 12.350 | 1.00 | 15.32 |
| ATOM | 2638 | O | PRO | 1151 | 23.899 | 25.098 | 13.148 | 1.00 | 15.15 |
| ATOM | 2639 | N | THR | 1152 | 25.172 | 26.765 | 12.400 | 1.00 | 16.47 |
| ATOM | 2640 | H | THR | 1152 | 25.335 | 27.411 | 11.696 | 1.00 | 0.00 |
| ATOM | 2641 | CA | THR | 1152 | 26.116 | 26.627 | 13.508 | 1.00 | 14.67 |
| ATOM | 2642 | CB | THR | 1152 | 27.529 | 27.066 | 13.193 | 1.00 | 12.31 |
| ATOM | 2643 | OG1 | THR | 1152 | 27.455 | 28.407 | 12.695 | 1.00 | 12.00 |
| ATOM | 2644 | HG1 | THR | 1152 | 28.289 | 28.535 | 12.202 | 1.00 | 0.00 |
| ATOM | 2645 | CG2 | THR | 1152 | 28.213 | 26.179 | 12.200 | 1.00 | 7.71 |
| ATOM | 2646 | C | THR | 1152 | 25.621 | 27.633 | 14.535 | 1.00 | 14.97 |
| ATOM | 2647 | O | THR | 1152 | 24.848 | 28.501 | 14.168 | 1.00 | 17.85 |
| ATOM | 2648 | N | PHE | 1153 | 26.046 | 27.603 | 15.800 | 1.00 | 15.54 |
| ATOM | 2649 | H | PHE | 1153 | 26.730 | 26.943 | 16.054 | 1.00 | 0.00 |
| ATOM | 2650 | CA | PHE | 1153 | 25.606 | 28.613 | 16.804 | 1.00 | 17.00 |
| ATOM | 2651 | CB | PHE | 1153 | 26.069 | 28.217 | 18.258 | 1.00 | 14.51 |
| ATOM | 2652 | CG | PHE | 1153 | 25.211 | 27.096 | 18.786 | 1.00 | 15.81 |
| ATOM | 2653 | CD1 | PHE | 1153 | 23.840 | 27.349 | 19.015 | 1.00 | 10.75 |
| ATOM | 2654 | CD2 | PHE | 1153 | 25.782 | 25.772 | 18.957 | 1.00 | 11.09 |
| ATOM | 2655 | CE1 | PHE | 1153 | 23.039 | 26.268 | 19.403 | 1.00 | 11.98 |
| ATOM | 2656 | CE2 | PHE | 1153 | 24.936 | 24.731 | 19.351 | 1.00 | 9.16 |
| ATOM | 2657 | CZ | PHE | 1153 | 23.581 | 24.977 | 19.570 | 1.00 | 8.97 |
| ATOM | 2658 | C | PHE | 1153 | 26.165 | 29.966 | 16.423 | 1.00 | 14.16 |
| ATOM | 2659 | O | PHE | 1153 | 25.521 | 30.945 | 16.680 | 1.00 | 15.63 |
| ATOM | 2660 | N | SER | 1154 | 27.320 | 30.114 | 15.800 | 1.00 | 14.92 |
| ATOM | 2661 | H | SER | 1154 | 27.895 | 29.338 | 15.792 | 1.00 | 0.00 |
| ATOM | 2662 | CA | SER | 1154 | 27.736 | 31.486 | 15.375 | 1.00 | 14.27 |
| ATOM | 2663 | CB | SER | 1154 | 29.044 | 31.464 | 14.746 | 1.00 | 14.85 |
| ATOM | 2664 | OG | SER | 1154 | 29.893 | 31.042 | 15.750 | 1.00 | 13.56 |
| ATOM | 2665 | HG | SER | 1154 | 30.801 | 30.970 | 15.428 | 1.00 | 0.00 |
| ATOM | 2666 | C | SER | 1154 | 26.795 | 32.115 | 14.361 | 1.00 | 14.52 |
| ATOM | 2667 | O | SER | 1154 | 26.594 | 33.336 | 14.257 | 1.00 | 15.87 |
| ATOM | 2668 | N | GLU | 1155 | 26.300 | 31.269 | 13.473 | 1.00 | 15.13 |
| ATOM | 2669 | H | GLU | 1155 | 26.596 | 30.332 | 13.454 | 1.00 | 0.00 |
| ATOM | 2670 | CA | GLU | 1155 | 25.276 | 31.693 | 12.478 | 1.00 | 17.25 |
| ATOM | 2671 | CB | GLU | 1155 | 24.930 | 30.591 | 11.512 | 1.00 | 19.41 |
| ATOM | 2672 | CG | GLU | 1155 | 26.145 | 30.385 | 10.623 | 1.00 | 25.47 |
| ATOM | 2673 | CD | GLU | 1155 | 25.622 | 29.339 | 9.734 | 1.00 | 30.50 |
| ATOM | 2674 | OE1 | GLU | 1155 | 26.032 | 28.190 | 9.920 | 1.00 | 31.07 |
| ATOM | 2675 | OE2 | GLU | 1155 | 24.760 | 29.711 | 8.915 | 1.00 | 37.89 |
| ATOM | 2676 | C | GLU | 1155 | 23.972 | 32.053 | 13.159 | 1.00 | 16.18 |
| ATOM | 2677 | O | GLU | 1155 | 23.263 | 32.907 | 12.695 | 1.00 | 17.16 |
| ATOM | 2678 | N | LEU | 1156 | 23.516 | 31.239 | 14.128 | 1.00 | 16.34 |
| ATOM | 2679 | H | LEU | 1156 | 23.987 | 30.393 | 14.261 | 1.00 | 0.00 |
| ATOM | 2680 | CA | LEU | 1156 | 22.304 | 31.541 | 14.911 | 1.00 | 16.18 |
| ATOM | 2681 | CB | LEU | 1156 | 21.961 | 30.337 | 15.845 | 1.00 | 13.12 |
| ATOM | 2682 | CG | LEU | 1156 | 21.474 | 29.032 | 15.136 | 1.00 | 8.09 |
| ATOM | 2683 | CD1 | LEU | 1156 | 21.652 | 27.786 | 16.030 | 1.00 | 8.53 |
| ATOM | 2684 | CD2 | LEU | 1156 | 20.043 | 29.287 | 14.708 | 1.00 | 8.89 |
| ATOM | 2685 | C | LEU | 1156 | 22.480 | 32.851 | 15.740 | 1.00 | 15.95 |
| ATOM | 2686 | O | LEU | 1156 | 21.508 | 33.576 | 15.882 | 1.00 | 16.98 |
| ATOM | 2687 | N | VAL | 1157 | 23.632 | 33.188 | 16.369 | 1.00 | 15.59 |
| ATOM | 2688 | H | VAL | 1157 | 24.329 | 32.506 | 16.404 | 1.00 | 0.00 |
| ATOM | 2689 | CA | VAL | 1157 | 23.822 | 34.492 | 17.100 | 1.00 | 14.43 |
| ATOM | 2690 | CB | VAL | 1157 | 25.221 | 34.480 | 17.735 | 1.00 | 13.22 |
| ATOM | 2691 | CG1 | VAL | 1157 | 25.615 | 35.884 | 18.221 | 1.00 | 12.88 |
| ATOM | 2692 | CG2 | VAL | 1157 | 25.235 | 33.484 | 18.884 | 1.00 | 10.65 |
| ATOM | 2693 | C | VAL | 1157 | 23.673 | 35.653 | 16.112 | 1.00 | 15.13 |
| ATOM | 2694 | O | VAL | 1157 | 22.988 | 36.641 | 16.347 | 1.00 | 16.47 |
| ATOM | 2695 | N | GLU | 1158 | 24.323 | 35.552 | 14.934 | 1.00 | 17.34 |
| ATOM | 2696 | H | GLU | 1158 | 24.933 | 34.797 | 14.822 | 1.00 | 0.00 |
| ATOM | 2697 | CA | GLU | 1158 | 24.190 | 36.600 | 13.867 | 1.00 | 19.28 |
| ATOM | 2698 | CB | GLU | 1158 | 24.998 | 36.219 | 12.575 | 1.00 | 18.27 |
| ATOM | 2699 | CG | GLU | 1158 | 25.111 | 37.309 | 11.454 | 1.00 | 24.47 |
| ATOM | 2700 | CD | GLU | 1158 | 26.067 | 36.946 | 10.254 | 1.00 | 26.69 |
| ATOM | 2701 | OE1 | GLU | 1158 | 26.798 | 35.940 | 10.251 | 1.00 | 30.34 |
| ATOM | 2702 | OE2 | GLU | 1158 | 26.092 | 37.701 | 9.283 | 1.00 | 29.62 |
| ATOM | 2703 | C | GLU | 1158 | 22.716 | 36.764 | 13.480 | 1.00 | 17.63 |
| ATOM | 2704 | O | GLU | 1158 | 22.091 | 37.784 | 13.691 | 1.00 | 19.03 |
| ATOM | 2705 | N | HIS | 1159 | 22.084 | 35.696 | 13.031 | 1.00 | 16.20 |
| ATOM | 2706 | H | HIS | 1159 | 22.596 | 34.865 | 12.881 | 1.00 | 0.00 |
| ATOM | 2707 | CA | HIS | 1159 | 20.689 | 35.696 | 12.636 | 1.00 | 14.60 |
| ATOM | 2708 | C | HIS | 1159 | 19.789 | 36.188 | 13.768 | 1.00 | 15.35 |
| ATOM | 2709 | O | HIS | 1159 | 18.924 | 37.071 | 13.622 | 1.00 | 14.49 |
| ATOM | 2710 | CB | HIS | 1159 | 20.323 | 34.278 | 12.240 | 1.00 | 13.01 |
| ATOM | 2711 | CG | HIS | 1159 | 19.014 | 34.354 | 11.648 | 1.00 | 17.91 |
| ATOM | 2712 | ND1 | HIS | 1159 | 17.979 | 33.488 | 11.949 | 1.00 | 22.96 |
| ATOM | 2713 | HD1 | HIS | 1159 | 18.056 | 32.663 | 12.464 | 1.00 | 0.00 |
| ATOM | 2714 | CD2 | HIS | 1159 | 18.557 | 35.211 | 10.628 | 1.00 | 16.00 |
| ATOM | 2715 | NE2 | HIS | 1159 | 17.256 | 34.848 | 10.348 | 1.00 | 17.03 |
| ATOM | 2716 | CE1 | HIS | 1159 | 16.872 | 33.806 | 11.146 | 1.00 | 17.18 |
| ATOM | 2717 | N | LEU | 1160 | 19.907 | 35.542 | 14.897 | 1.00 | 13.31 |
| ATOM | 2718 | H | LEU | 1160 | 20.518 | 34.792 | 14.963 | 1.00 | 0.00 |
| ATOM | 2719 | CA | LEU | 1160 | 19.085 | 35.991 | 16.006 | 1.00 | 16.11 |
| ATOM | 2720 | CB | LEU | 1160 | 19.311 | 35.075 | 17.251 | 1.00 | 16.34 |
| ATOM | 2721 | CG | LEU | 1160 | 18.622 | 33.689 | 17.024 | 1.00 | 18.24 |
| ATOM | 2722 | CD1 | LEU | 1160 | 19.072 | 32.711 | 18.077 | 1.00 | 18.44 |
| ATOM | 2723 | CD2 | LEU | 1160 | 17.109 | 33.835 | 17.055 | 1.00 | 20.73 |
| ATOM | 2724 | C | LEU | 1160 | 19.346 | 37.457 | 16.339 | 1.00 | 18.55 |
| ATOM | 2725 | O | LEU | 1160 | 18.396 | 38.168 | 16.648 | 1.00 | 20.49 |
| ATOM | 2726 | N | GLY | 1161 | 20.580 | 37.976 | 16.312 | 1.00 | 19.84 |
| ATOM | 2727 | H | GLY | 1161 | 21.312 | 37.400 | 16.023 | 1.00 | 0.00 |
| ATOM | 2728 | CA | GLY | 1161 | 20.863 | 39.385 | 16.614 | 1.00 | 18.37 |
| ATOM | 2729 | C | GLY | 1161 | 20.155 | 40.241 | 15.612 | 1.00 | 17.78 |
| ATOM | 2730 | O | GLY | 1161 | 19.671 | 41.292 | 15.960 | 1.00 | 15.80 |
| ATOM | 2731 | N | ASN | 1162 | 20.057 | 39.838 | 14.351 | 1.00 | 18.13 |
| ATOM | 2732 | H | ASN | 1162 | 20.570 | 39.042 | 14.104 | 1.00 | 0.00 |
| ATOM | 2733 | CA | ASN | 1162 | 19.352 | 40.634 | 13.295 | 1.00 | 19.21 |
| ATOM | 2734 | CB | ASN | 1162 | 19.550 | 40.094 | 11.843 | 1.00 | 15.14 |
| ATOM | 2735 | CG | ASN | 1162 | 20.989 | 40.177 | 11.399 | 1.00 | 19.35 |
| ATOM | 2736 | OD1 | ASN | 1162 | 21.817 | 40.972 | 11.842 | 1.00 | 23.33 |
| ATOM | 2737 | ND2 | ASN | 1162 | 21.422 | 39.362 | 10.451 | 1.00 | 21.29 |
| ATOM | 2738 | HD21 | ASN | 1162 | 20.890 | 38.702 | 9.992 | 1.00 | 0.00 |
| ATOM | 2739 | HD22 | ASN | 1162 | 22.371 | 39.478 | 10.224 | 1.00 | 0.00 |
| ATOM | 2740 | C | ASN | 1162 | 17.866 | 40.625 | 13.536 | 1.00 | 20.19 |
| ATOM | 2741 | O | ASN | 1162 | 17.167 | 41.598 | 13.241 | 1.00 | 22.01 |
| ATOM | 2742 | N | LEU | 1163 | 17.295 | 39.471 | 13.953 | 1.00 | 20.95 |
| ATOM | 2743 | H | LEU | 1163 | 17.857 | 38.669 | 14.014 | 1.00 | 0.00 |
| ATOM | 2744 | CA | LEU | 1163 | 15.831 | 39.404 | 14.271 | 1.00 | 19.69 |
| ATOM | 2745 | CB | LEU | 1163 | 15.390 | 37.947 | 14.590 | 1.00 | 19.49 |
| ATOM | 2746 | CG | LEU | 1163 | 14.869 | 37.122 | 13.466 | 1.00 | 20.62 |
| ATOM | 2747 | CD1 | LEU | 1163 | 15.446 | 37.558 | 12.130 | 1.00 | 24.13 |
| ATOM | 2748 | CD2 | LEU | 1163 | 15.096 | 35.685 | 13.860 | 1.00 | 24.67 |
| ATOM | 2749 | C | LEU | 1163 | 15.567 | 40.291 | 15.516 | 1.00 | 22.07 |
| ATOM | 2750 | O | LEU | 1163 | 14.601 | 41.019 | 15.675 | 1.00 | 21.23 |
| ATOM | 2751 | N | LEU | 1164 | 16.445 | 40.290 | 16.496 | 1.00 | 23.74 |
| ATOM | 2752 | H | LEU | 1164 | 17.184 | 39.654 | 16.447 | 1.00 | 0.00 |
| ATOM | 2753 | CA | LEU | 1164 | 16.220 | 41.147 | 17.689 | 1.00 | 26.17 |
| ATOM | 2754 | CB | LEU | 1164 | 17.342 | 40.917 | 18.584 | 1.00 | 22.57 |
| ATOM | 2755 | CG | LEU | 1164 | 17.031 | 41.192 | 19.990 | 1.00 | 25.46 |
| ATOM | 2756 | CD1 | LEU | 1164 | 15.684 | 40.781 | 20.519 | 1.00 | 20.21 |
| ATOM | 2757 | CD2 | LEU | 1164 | 18.146 | 40.368 | 20.661 | 1.00 | 29.71 |
| ATOM | 2758 | C | LEU | 1164 | 16.149 | 42.609 | 17.305 | 1.00 | 28.67 |
| ATOM | 2759 | O | LEU | 1164 | 15.414 | 43.406 | 17.879 | 1.00 | 29.89 |
| ATOM | 2760 | N | GLN | 1165 | 17.067 | 42.978 | 16.411 | 1.00 | 30.02 |
| ATOM | 2761 | H | GLN | 1165 | 17.734 | 42.318 | 16.158 | 1.00 | 0.00 |
| ATOM | 2762 | CA | GLN | 1165 | 17.182 | 44.325 | 15.869 | 1.00 | 31.77 |
| ATOM | 2763 | CB | GLN | 1165 | 18.418 | 44.424 | 14.914 | 1.00 | 34.55 |
| ATOM | 2764 | CG | GLN | 1165 | 19.493 | 45.400 | 15.354 | 1.00 | 36.09 |
| ATOM | 2765 | CD | GLN | 1165 | 18.783 | 46.641 | 15.924 | 1.00 | 41.02 |
| ATOM | 2766 | OE1 | GLN | 1165 | 18.740 | 46.954 | 17.114 | 1.00 | 42.70 |
| ATOM | 2767 | NE2 | GLN | 1165 | 18.126 | 47.464 | 15.134 | 1.00 | 36.43 |
| ATOM | 2768 | HE21 | GLN | 1165 | 18.051 | 47.350 | 14.178 | 1.00 | 0.00 |
| ATOM | 2769 | HE22 | GLN | 1165 | 17.709 | 48.194 | 15.634 | 1.00 | 0.00 |
| ATOM | 2770 | C | GLN | 1165 | 15.903 | 44.663 | 15.127 | 1.00 | 33.32 |
| ATOM | 2771 | O | GLN | 1165 | 15.556 | 45.836 | 14.951 | 1.00 | 34.83 |
| ATOM | 2772 | N | ALA | 1166 | 15.302 | 43.648 | 14.531 | 1.00 | 34.66 |
| ATOM | 2773 | H | ALA | 1166 | 15.632 | 42.736 | 14.597 | 1.00 | 0.00 |
| ATOM | 2774 | CA | ALA | 1166 | 14.034 | 43.911 | 13.861 | 1.00 | 37.31 |
| ATOM | 2775 | CB | ALA | 1166 | 13.947 | 42.710 | 12.865 | 1.00 | 31.15 |
| ATOM | 2776 | C | ALA | 1166 | 12.772 | 44.215 | 14.805 | 1.00 | 43.04 |
| ATOM | 2777 | O | ALA | 1166 | 12.511 | 43.659 | 15.923 | 1.00 | 37.20 |
| ATOM | 2779 | O01 | AGI | 1 | 22.581 | 24.990 | 39.854 | 1.00 | 16.66 |
| ATOM | 2780 | C02 | AGI | 1 | 21.643 | 25.593 | 38.942 | 1.00 | 16.79 |
| ATOM | 2781 | C03 | AGI | 1 | 25.810 | 23.097 | 40.054 | 1.00 | 12.94 |
| ATOM | 2782 | C04 | AGI | 1 | 26.039 | 22.840 | 38.704 | 1.00 | 15.87 |
| ATOM | 2783 | C05 | AGI | 1 | 25.072 | 23.214 | 37.734 | 1.00 | 15.52 |
| ATOM | 2784 | C06 | AGI | 1 | 23.863 | 23.848 | 38.086 | 1.00 | 15.54 |
| ATOM | 2785 | C07 | AGI | 1 | 23.635 | 24.109 | 39.425 | 1.00 | 15.22 |
| ATOM | 2786 | C08 | AGI | 1 | 24.610 | 23.745 | 40.392 | 1.00 | 15.93 |
| ATOM | 2787 | C09 | AGI | 1 | 25.336 | 23.054 | 36.320 | 1.00 | 13.68 |
| ATOM | 2788 | O10 | AGI | 1 | 24.581 | 23.581 | 35.521 | 1.00 | 16.24 |
| ATOM | 2789 | N11 | AGI | 1 | 26.326 | 22.271 | 35.893 | 1.00 | 11.13 |
| ATOM | 2790 | C12 | AGI | 1 | 26.999 | 22.855 | 32.377 | 1.00 | 18.19 |
| ATOM | 2791 | C13 | AGI | 1 | 27.526 | 21.562 | 32.037 | 1.00 | 22.15 |
| ATOM | 2792 | C14 | AGI | 1 | 27.629 | 20.563 | 33.012 | 1.00 | 17.14 |
| ATOM | 2793 | C15 | AGI | 1 | 27.209 | 20.832 | 34.297 | 1.00 | 15.75 |
| ATOM | 2794 | C16 | AGI | 1 | 26.696 | 22.062 | 34.638 | 1.00 | 13.81 |
| ATOM | 2795 | C17 | AGI | 1 | 26.598 | 23.049 | 33.687 | 1.00 | 16.26 |
| ATOM | 2796 | C19 | AGI | 1 | 20.848 | 26.696 | 39.600 | 1.00 | 17.42 |
| ATOM | 2797 | N20 | AGI | 1 | 21.380 | 27.920 | 39.764 | 1.00 | 17.28 |
| ATOM | 2798 | N21 | AGI | 1 | 20.407 | 28.768 | 40.248 | 1.00 | 17.95 |
| ATOM | 2799 | C22 | AGI | 1 | 19.311 | 28.072 | 40.381 | 1.00 | 17.31 |
| ATOM | 2800 | C23 | AGI | 1 | 19.497 | 26.753 | 39.978 | 1.00 | 17.39 |
| ATOM | 2801 | N24 | AGI | 1 | 18.007 | 28.598 | 40.723 | 1.00 | 19.82 |
| ATOM | 2802 | C25 | AGI | 1 | 16.858 | 27.971 | 40.907 | 1.00 | 22.93 |
| ATOM | 2803 | C26 | AGI | 1 | 16.601 | 26.661 | 40.456 | 1.00 | 24.71 |
| ATOM | 2804 | N27 | AGI | 1 | 15.429 | 26.101 | 40.663 | 1.00 | 23.52 |
| ATOM | 2805 | C28 | AGI | 1 | 14.481 | 26.797 | 41.322 | 1.00 | 24.68 |
| ATOM | 2806 | C29 | AGI | 1 | 14.624 | 28.102 | 41.767 | 1.00 | 18.61 |
| ATOM | 2807 | C30 | AGI | 1 | 15.845 | 28.696 | 41.548 | 1.00 | 21.59 |
| ATOM | 2808 | O31 | AGI | 1 | 13.225 | 26.195 | 41.356 | 1.00 | 29.16 |
| ATOM | 2809 | C32 | AGI | 1 | 13.124 | 24.983 | 40.581 | 1.00 | 37.28 |
| ATOM | 2810 | C34 | AGI | 1 | 26.866 | 24.022 | 31.466 | 1.00 | 17.98 |
| ATOM | 2811 | N35 | AGI | 1 | 28.124 | 21.233 | 30.792 | 1.00 | 24.65 |
| ATOM | 2812 | C36 | AGI | 1 | 29.442 | 21.338 | 30.508 | 1.00 | 24.97 |
| ATOM | 2813 | C37 | AGI | 1 | 27.523 | 20.851 | 29.629 | 1.00 | 25.52 |
| ATOM | 2814 | F38 | AGI | 1 | 28.039 | 24.363 | 30.902 | 1.00 | 18.90 |
| ATOM | 2815 | F39 | AGI | 1 | 25.949 | 23.826 | 30.522 | 1.00 | 17.17 |
| ATOM | 2816 | F40 | AGI | 1 | 26.494 | 25.102 | 32.144 | 1.00 | 21.67 |
| ATOM | 2817 | N41 | AGI | 1 | 28.435 | 20.729 | 28.626 | 1.00 | 24.59 |
| ATOM | 2818 | C71 | AGI | 1 | 29.616 | 21.046 | 29.146 | 1.00 | 24.41 |
| ATOM | 2819 | F72 | AGI | 1 | 24.419 | 24.059 | 41.674 | 1.00 | 14.22 |
| ATOM | 2820 | H47 | AGI | 1 | 26.860 | 21.891 | 36.600 | 1.00 | 0.00 |
| ATOM | 2821 | H51 | AGI | 1 | 22.315 | 28.179 | 39.613 | 1.00 | 0.00 |
| ATOM | 2822 | H53 | AGI | 1 | 17.963 | 29.571 | 40.837 | 1.00 | 0.00 |
| ATOM | 2823 | OH2 | WAT | 1 | 21.400 | 15.094 | 31.045 | 1.00 | 22.93 |
| ATOM | 2826 | OH2 | WAT | 2 | 19.425 | 11.225 | 30.668 | 1.00 | 25.96 |
| ATOM | 2829 | OH2 | WAT | 3 | 21.340 | 12.536 | 34.686 | 1.00 | 17.84 |
| ATOM | 2832 | OH2 | WAT | 4 | 19.657 | 12.032 | 36.567 | 1.00 | 29.56 |
| ATOM | 2835 | OH2 | WAT | 5 | 20.130 | 11.164 | 40.355 | 1.00 | 15.79 |
| ATOM | 2838 | OH2 | WAT | 6 | 38.456 | 24.879 | 52.594 | 1.00 | 36.99 |
| ATOM | 2841 | OH2 | WAT | 7 | 41.194 | 28.067 | 35.609 | 1.00 | 33.41 |
| ATOM | 2844 | OH2 | WAT | 8 | 36.265 | 23.160 | 51.508 | 1.00 | 20.75 |
| ATOM | 2847 | OH2 | WAT | 9 | 20.851 | 13.323 | 29.065 | 1.00 | 38.90 |
| ATOM | 2850 | OH2 | WAT | 10 | 41.762 | 23.986 | 37.979 | 1.00 | 21.57 |
| ATOM | 2853 | OH2 | WAT | 11 | 30.544 | 26.901 | 34.010 | 1.00 | 54.58 |
| ATOM | 2856 | OH2 | WAT | 12 | 28.451 | 15.178 | 47.258 | 1.00 | 9.68 |
| ATOM | 2859 | OH2 | WAT | 13 | 30.460 | 3.539 | 45.592 | 1.00 | 21.26 |
| ATOM | 2862 | OH2 | WAT | 14 | 33.940 | 0.946 | 40.551 | 1.00 | 4.70 |
| ATOM | 2865 | OH2 | WAT | 15 | 35.968 | 6.373 | 36.177 | 1.00 | 24.22 |
| ATOM | 2868 | OH2 | WAT | 16 | 35.226 | 9.477 | 35.278 | 1.00 | 51.52 |
| ATOM | 2871 | OH2 | WAT | 17 | 37.571 | 13.408 | 37.243 | 1.00 | 30.00 |
| ATOM | 2874 | OH2 | WAT | 18 | 12.569 | 21.510 | 28.708 | 1.00 | 13.07 |
| ATOM | 2877 | OH2 | WAT | 19 | 8.671 | 23.622 | 27.576 | 1.00 | 17.97 |
| ATOM | 2880 | OH2 | WAT | 20 | 7.595 | 14.965 | 27.755 | 1.00 | 25.61 |
| ATOM | 2883 | OH2 | WAT | 21 | 10.790 | 28.469 | 36.897 | 1.00 | 18.48 |
| ATOM | 2886 | OH2 | WAT | 22 | −2.527 | 28.562 | 27.567 | 1.00 | 22.35 |
| ATOM | 2889 | OH2 | WAT | 23 | −1.844 | 37.437 | 28.067 | 1.00 | 27.08 |
| ATOM | 2892 | OH2 | WAT | 24 | 14.429 | 38.747 | 34.588 | 1.00 | 32.22 |
| ATOM | 2895 | OH2 | WAT | 25 | 15.299 | 41.405 | 37.396 | 1.00 | 32.27 |
| ATOM | 2898 | OH2 | WAT | 26 | 7.923 | 42.971 | 21.716 | 1.00 | 25.29 |
| ATOM | 2901 | OH2 | WAT | 27 | 3.115 | 25.355 | 10.833 | 1.00 | 28.14 |
| ATOM | 2904 | OH2 | WAT | 28 | 10.227 | 21.558 | 32.746 | 1.00 | 26.54 |
| ATOM | 2907 | OH2 | WAT | 29 | 19.486 | 30.207 | 10.202 | 1.00 | 28.95 |
| ATOM | 2910 | OH2 | WAT | 30 | 14.453 | 32.638 | 9.591 | 1.00 | 17.41 |
| ATOM | 2913 | OH2 | WAT | 31 | 11.205 | 37.188 | 13.909 | 1.00 | 21.75 |
| ATOM | 2916 | OH2 | WAT | 32 | 4.982 | 20.331 | 10.232 | 1.00 | 43.45 |
| ATOM | 2919 | OH2 | WAT | 33 | 17.179 | 24.649 | 6.341 | 1.00 | 28.28 |
| ATOM | 2922 | OH2 | WAT | 34 | 11.623 | 39.889 | 14.687 | 1.00 | 35.89 |
| ATOM | 2925 | OH2 | WAT | 35 | 11.339 | 39.971 | 11.260 | 1.00 | 40.88 |
| ATOM | 2928 | OH2 | WAT | 36 | 29.633 | 37.146 | 28.393 | 1.00 | 27.63 |
| ATOM | 2931 | OH2 | WAT | 37 | 35.868 | 33.440 | 30.026 | 1.00 | 30.10 |
| ATOM | 2934 | OH2 | WAT | 38 | 27.533 | 24.986 | 16.088 | 1.00 | 19.59 |
| ATOM | 2937 | OH2 | WAT | 39 | 29.319 | 28.258 | 16.624 | 1.00 | 16.81 |
| ATOM | 2940 | OH2 | WAT | 40 | 23.781 | 16.794 | 21.247 | 1.00 | 17.50 |
| ATOM | 2943 | OH2 | WAT | 41 | 19.170 | 16.113 | 23.745 | 1.00 | 13.71 |
| ATOM | 2946 | OH2 | WAT | 42 | 20.546 | 7.592 | 21.724 | 1.00 | 45.91 |
| ATOM | 2949 | OH2 | WAT | 43 | 15.869 | 22.633 | 7.299 | 1.00 | 18.39 |
| ATOM | 2952 | OH2 | WAT | 44 | 30.507 | 13.495 | 47.426 | 1.00 | 11.20 |
| ATOM | 2955 | OH2 | WAT | 45 | 36.403 | 28.744 | 58.203 | 1.00 | 39.64 |
| ATOM | 2958 | OH2 | WAT | 46 | 38.544 | 27.398 | 56.745 | 1.00 | 29.70 |
| ATOM | 2961 | OH2 | WAT | 47 | 13.063 | 20.681 | 9.314 | 1.00 | 23.53 |
| ATOM | 2964 | OH2 | WAT | 48 | 14.614 | 20.766 | 5.692 | 1.00 | 64.50 |
| ATOM | 2967 | OH2 | WAT | 49 | 14.317 | 15.435 | 5.814 | 1.00 | 48.17 |
| ATOM | 2970 | OH2 | WAT | 50 | 10.496 | 20.770 | 30.174 | 1.00 | 18.58 |
| ATOM | 2973 | OH2 | WAT | 51 | 1.321 | 23.079 | 27.109 | 1.00 | 20.63 |
| ATOM | 2976 | OH2 | WAT | 52 | 13.746 | 13.952 | 18.647 | 1.00 | 15.79 |
| ATOM | 2979 | OH2 | WAT | 53 | 20.805 | 15.439 | 20.029 | 1.00 | 28.31 |
| ATOM | 2982 | OH2 | WAT | 54 | 28.585 | 18.020 | 13.436 | 1.00 | 45.07 |
| ATOM | 2985 | OH2 | WAT | 55 | 25.707 | 15.518 | 14.814 | 1.00 | 39.11 |
| ATOM | 2988 | OH2 | WAT | 56 | 25.637 | 11.616 | 11.869 | 1.00 | 65.38 |
| ATOM | 2991 | OH2 | WAT | 57 | 3.040 | 17.846 | 17.201 | 1.00 | 33.20 |
| ATOM | 2994 | OH2 | WAT | 58 | 2.210 | 26.661 | 21.052 | 1.00 | 12.51 |
| ATOM | 2997 | OH2 | WAT | 59 | 25.674 | 8.909 | 12.371 | 1.00 | 37.85 |
| ATOM | 3000 | OH2 | WAT | 60 | 42.879 | 28.354 | 26.363 | 1.00 | 40.00 |
| ATOM | 3003 | OH2 | WAT | 61 | 41.495 | 26.856 | 37.868 | 1.00 | 26.84 |
| ATOM | 3006 | OH2 | WAT | 62 | 39.910 | 21.848 | 34.542 | 1.00 | 11.92 |
| ATOM | 3009 | OH2 | WAT | 63 | 41.932 | 26.200 | 27.111 | 1.00 | 36.10 |
| ATOM | 3012 | OH2 | WAT | 64 | 20.358 | 17.857 | 32.701 | 1.00 | 14.85 |
| ATOM | 3015 | OH2 | WAT | 65 | 20.099 | 12.911 | 32.457 | 1.00 | 15.26 |
| ATOM | 3018 | OH2 | WAT | 66 | 15.746 | 18.911 | 38.679 | 1.00 | 26.77 |
| ATOM | 3021 | OH2 | WAT | 67 | 20.002 | 37.361 | 9.196 | 1.00 | 20.39 |
| ATOM | 3024 | OH2 | WAT | 68 | 11.561 | 13.182 | 25.433 | 1.00 | 19.10 |
| ATOM | 3027 | OH2 | WAT | 69 | 37.807 | 29.085 | 34.930 | 1.00 | 26.93 |
| ATOM | 3030 | OH2 | WAT | 70 | 38.034 | 32.720 | 36.667 | 1.00 | 26.50 |
| ATOM | 3033 | OH2 | WAT | 71 | 21.330 | 12.775 | 26.497 | 1.00 | 50.64 |
| ATOM | 3036 | OH2 | WAT | 72 | 40.911 | 31.406 | 34.061 | 1.00 | 52.28 |
| ATOM | 3039 | OH2 | WAT | 73 | 13.428 | 16.134 | 39.067 | 1.00 | 40.88 |
| ATOM | 3042 | OH2 | WAT | 74 | 11.854 | 21.495 | 35.083 | 1.00 | 25.55 |
| ATOM | 3045 | OH2 | WAT | 75 | 20.227 | 12.458 | 51.144 | 1.00 | 29.24 |
| ATOM | 3048 | OH2 | WAT | 76 | 8.387 | 29.945 | 37.663 | 1.00 | 25.72 |
| ATOM | 3051 | OH2 | WAT | 77 | 5.126 | 33.659 | 37.350 | 1.00 | 42.19 |
| ATOM | 3054 | OH2 | WAT | 78 | 0.926 | 33.198 | 37.381 | 1.00 | 82.00 |
| ATOM | 3057 | OH2 | WAT | 79 | 22.788 | 37.140 | 47.076 | 1.00 | 20.62 |
| ATOM | 3060 | OH2 | WAT | 80 | 28.422 | 2.602 | 41.035 | 1.00 | 43.55 |
| ATOM | 3063 | OH2 | WAT | 81 | −1.367 | 21.107 | 21.842 | 1.00 | 43.55 |
| ATOM | 3066 | OH2 | WAT | 82 | 24.342 | 39.630 | 48.038 | 1.00 | 54.36 |
| ATOM | 3069 | OH2 | WAT | 83 | −0.780 | 25.339 | 18.469 | 1.00 | 47.73 |
| ATOM | 3072 | OH2 | WAT | 84 | 1.502 | 23.967 | 20.101 | 1.00 | 34.19 |
| ATOM | 3075 | OH2 | WAT | 85 | 2.141 | 23.794 | 17.505 | 1.00 | 50.27 |
| ATOM | 3078 | OH2 | WAT | 86 | 29.188 | 33.645 | 22.250 | 1.00 | 18.29 |
| ATOM | 3081 | OH2 | WAT | 87 | 32.012 | 33.308 | 22.855 | 1.00 | 36.00 |
| ATOM | 3084 | OH2 | WAT | 88 | 36.492 | 31.032 | 23.519 | 1.00 | 42.81 |
| ATOM | 3087 | OH2 | WAT | 89 | 31.678 | 28.677 | 18.496 | 1.00 | 17.48 |
| ATOM | 3090 | OH2 | WAT | 90 | 30.000 | 25.753 | 16.377 | 1.00 | 33.66 |
| ATOM | 3093 | OH2 | WAT | 91 | 7.414 | 6.354 | 25.176 | 1.00 | 45.95 |
| ATOM | 3096 | OH2 | WAT | 92 | 8.145 | 21.093 | 28.714 | 1.00 | 23.34 |
| ATOM | 3099 | OH2 | WAT | 93 | 4.681 | 26.807 | 35.437 | 1.00 | 34.41 |
| ATOM | 3102 | OH2 | WAT | 94 | −4.670 | 27.121 | 27.969 | 1.00 | 7.66 |
| ATOM | 3105 | OH2 | WAT | 95 | 15.897 | 30.820 | 8.074 | 1.00 | 17.41 |
| ATOM | 3108 | OH2 | WAT | 96 | 20.800 | 27.309 | 7.802 | 1.00 | 29.75 |
| ATOM | 3111 | OH2 | WAT | 97 | 19.327 | 32.337 | 7.534 | 1.00 | 46.08 |
| ATOM | 3114 | OH2 | WAT | 98 | 16.392 | 43.067 | 24.309 | 1.00 | 47.31 |
| ATOM | 3117 | OH2 | WAT | 99 | 14.351 | 42.324 | 26.504 | 1.00 | 46.39 |
| ATOM | 3120 | OH2 | WAT | 100 | 18.815 | 40.297 | 24.345 | 1.00 | 15.27 |
| ATOM | 3123 | OH2 | WAT | 101 | 31.775 | 36.166 | 32.325 | 1.00 | 31.35 |
| ATOM | 3126 | OH2 | WAT | 102 | 35.715 | 39.142 | 32.148 | 1.00 | 39.28 |
| ATOM | 3129 | OH2 | WAT | 103 | 32.889 | 40.771 | 31.188 | 1.00 | 42.39 |
| ATOM | 3132 | OH2 | WAT | 104 | 36.867 | 31.193 | 29.065 | 1.00 | 23.94 |
| ATOM | 3135 | OH2 | WAT | 105 | 17.566 | 17.625 | 25.778 | 1.00 | 18.98 |
| ATOM | 3138 | OH2 | WAT | 106 | 21.398 | 19.009 | 23.367 | 1.00 | 21.53 |
| ATOM | 3141 | OH2 | WAT | 107 | 27.101 | 13.921 | 12.898 | 1.00 | 26.04 |
| ATOM | 3144 | OH2 | WAT | 108 | 6.559 | 22.545 | 8.817 | 1.00 | 37.57 |
| ATOM | 3147 | OH2 | WAT | 109 | 2.404 | 33.695 | 16.329 | 1.00 | 32.60 |
| ATOM | 3150 | OH2 | WAT | 110 | 30.800 | 18.720 | 17.732 | 1.00 | 36.44 |
| ATOM | 3153 | OH2 | WAT | 111 | 32.266 | 16.203 | 18.052 | 1.00 | 28.37 |
| ATOM | 3156 | OH2 | WAT | 112 | 10.955 | 26.078 | 38.607 | 1.00 | 34.45 |
| ATOM | 3159 | OH2 | WAT | 113 | 45.573 | 22.495 | 38.903 | 1.00 | 35.85 |
| ATOM | 3162 | OH2 | WAT | 114 | 23.554 | 33.882 | 10.094 | 1.00 | 20.02 |
| ATOM | 3165 | OH2 | WAT | 115 | −7.644 | 36.627 | 32.108 | 1.00 | 33.76 |
| ATOM | 3168 | OH2 | WAT | 116 | −6.579 | 37.715 | 34.359 | 1.00 | 31.08 |
| ATOM | 3171 | OH2 | WAT | 117 | 1.439 | 22.456 | 22.255 | 1.00 | 26.66 |
| ATOM | 3174 | OH2 | WAT | 118 | 14.067 | 30.888 | 44.225 | 1.00 | 49.65 |
| ATOM | 3177 | OH2 | WAT | 119 | 23.627 | 38.167 | 35.878 | 1.00 | 32.18 |
| ATOM | 3180 | OH2 | WAT | 120 | 8.872 | 12.471 | 26.415 | 1.00 | 25.08 |
| ATOM | 3183 | OH2 | WAT | 121 | 20.205 | 3.862 | 25.679 | 1.00 | 29.23 |
| ATOM | 3186 | OH2 | WAT | 122 | 7.792 | 18.309 | 28.535 | 1.00 | 24.72 |
| ATOM | 3189 | OH2 | WAT | 123 | 7.230 | 22.937 | 36.086 | 1.00 | 54.52 |
| ATOM | 3192 | OH2 | WAT | 124 | 17.059 | 27.997 | 51.101 | 1.00 | 42.63 |
| ATOM | 3195 | OH2 | WAT | 125 | 20.605 | 29.282 | 53.106 | 1.00 | 32.36 |
| ATOM | 3198 | OH2 | WAT | 126 | 24.621 | 23.938 | 56.498 | 1.00 | 43.67 |
| ATOM | 3201 | OH2 | WAT | 127 | 36.692 | 32.140 | 46.931 | 1.00 | 38.37 |
| ATOM | 3204 | OH2 | WAT | 128 | 30.621 | 36.680 | 48.150 | 1.00 | 21.14 |
| ATOM | 3207 | OH2 | WAT | 129 | 36.269 | 41.301 | 46.523 | 1.00 | 29.26 |
| ATOM | 3210 | OH2 | WAT | 130 | 36.205 | 37.173 | 47.745 | 1.00 | 53.21 |
| ATOM | 3213 | OH2 | WAT | 131 | 5.529 | 28.982 | 38.678 | 1.00 | 78.87 |
| ATOM | 3216 | OH2 | WAT | 132 | 2.253 | 20.712 | 24.095 | 1.00 | 19.33 |
| ATOM | 3219 | OH2 | WAT | 133 | 0.531 | 24.389 | 24.135 | 1.00 | 25.62 |
| ATOM | 3222 | OH2 | WAT | 134 | 8.227 | 29.872 | 7.959 | 1.00 | 39.23 |
| ATOM | 3225 | OH2 | WAT | 135 | 15.449 | 17.965 | 6.387 | 1.00 | 37.51 |
| ATOM | 3228 | OH2 | WAT | 136 | 15.038 | 14.100 | 8.724 | 1.00 | 35.56 |
| ATOM | 3231 | OH2 | WAT | 137 | 24.768 | 14.954 | 6.638 | 1.00 | 30.06 |
| ATOM | 3234 | OH2 | WAT | 138 | 27.417 | 14.777 | 8.898 | 1.00 | 36.79 |
| ATOM | 3237 | OH2 | WAT | 139 | 32.940 | 32.679 | 20.535 | 1.00 | 38.18 |
| ATOM | 3240 | OH2 | WAT | 140 | 29.393 | 36.401 | 23.686 | 1.00 | 28.09 |
| ATOM | 3243 | OH2 | WAT | 141 | 41.286 | 22.940 | 32.431 | 1.00 | 46.39 |
| ATOM | 3246 | OH2 | WAT | 142 | 44.351 | 28.056 | 37.461 | 1.00 | 54.07 |
| ATOM | 3249 | OH2 | WAT | 143 | 44.549 | 30.912 | 36.915 | 1.00 | 35.83 |
| ATOM | 3252 | OH2 | WAT | 144 | 45.137 | 31.749 | 39.579 | 1.00 | 42.05 |
| ATOM | 3255 | OH2 | WAT | 145 | 19.761 | 9.348 | 38.127 | 1.00 | 42.22 |
| ATOM | 3258 | OH2 | WAT | 146 | 23.945 | 8.207 | 37.578 | 1.00 | 41.26 |
| ATOM | 3261 | OH2 | WAT | 147 | 23.828 | 13.796 | 34.371 | 1.00 | 29.35 |
| ATOM | 3264 | OH2 | WAT | 148 | 25.179 | 16.087 | 37.292 | 1.00 | 18.88 |
| ATOM | 3267 | OH2 | WAT | 149 | 27.551 | 15.302 | 37.684 | 1.00 | 19.93 |
| ATOM | 3270 | OH2 | WAT | 150 | 27.545 | 14.162 | 35.163 | 1.00 | 45.83 |
| ATOM | 3273 | OH2 | WAT | 151 | 30.144 | 14.715 | 34.658 | 1.00 | 31.23 |
| ATOM | 3276 | OH2 | WAT | 152 | 31.117 | 7.978 | 36.709 | 1.00 | 24.33 |
| ATOM | 3279 | OH2 | WAT | 153 | 32.536 | 10.111 | 35.494 | 1.00 | 38.47 |
| ATOM | 3282 | OH2 | WAT | 154 | 35.384 | 7.731 | 31.998 | 1.00 | 27.78 |
| ATOM | 3285 | OH2 | WAT | 155 | 32.058 | 12.219 | 33.534 | 1.00 | 50.41 |
| ATOM | 3288 | OH2 | WAT | 156 | 23.379 | 3.450 | 43.753 | 1.00 | 42.28 |
| ATOM | 3291 | OH2 | WAT | 157 | 24.214 | 17.003 | 29.578 | 1.00 | 43.28 |
| ATOM | 3294 | OH2 | WAT | 158 | 23.568 | 15.432 | 32.311 | 1.00 | 26.14 |
| ATOM | 3297 | OH2 | WAT | 159 | 19.100 | 15.334 | 33.790 | 1.00 | 14.55 |
| ATOM | 3300 | OH2 | WAT | 160 | 27.245 | 8.720 | 37.332 | 1.00 | 31.35 |
| ATOM | 3303 | OH2 | WAT | 161 | 28.569 | 8.004 | 34.705 | 1.00 | 48.08 |
| ATOM | 3306 | OH2 | WAT | 162 | 36.172 | 31.123 | 35.274 | 1.00 | 24.65 |
| ATOM | 3309 | OH2 | WAT | 163 | 36.846 | 36.393 | 29.950 | 1.00 | 42.07 |
| ATOM | 3312 | OH2 | WAT | 164 | 37.499 | 10.359 | 37.930 | 1.00 | 36.50 |
| ATOM | 3315 | OH2 | WAT | 165 | 35.754 | 29.738 | 45.821 | 1.00 | 24.24 |
| ATOM | 3318 | OH2 | WAT | 166 | 34.583 | 13.981 | 49.592 | 1.00 | 22.44 |
| ATOM | 3321 | OH2 | WAT | 167 | 28.355 | 22.366 | 54.343 | 1.00 | 44.69 |
| ATOM | 3324 | OH2 | WAT | 168 | 25.218 | 7.363 | 49.242 | 1.00 | 31.91 |
| ATOM | 3327 | OH2 | WAT | 169 | 25.394 | 8.715 | 51.592 | 1.00 | 37.30 |
| ATOM | 3330 | OH2 | WAT | 170 | 23.407 | 35.493 | 41.806 | 1.00 | 44.65 |
| ATOM | 3333 | OH2 | WAT | 171 | 13.548 | 24.364 | 5.696 | 1.00 | 35.54 |
| ATOM | 3336 | OH2 | WAT | 172 | 16.095 | 28.941 | 5.737 | 1.00 | 54.96 |
| ATOM | 3339 | OH2 | WAT | 173 | 32.740 | 5.837 | 36.914 | 1.00 | 29.69 |
| ATOM | 3342 | OH2 | WAT | 174 | 0.926 | 28.119 | 15.089 | 1.00 | 45.75 |
| ATOM | 3345 | OH2 | WAT | 175 | 2.496 | 20.839 | 27.362 | 1.00 | 39.33 |
| ATOM | 3348 | OH2 | WAT | 176 | 3.425 | 16.659 | 24.014 | 1.00 | 40.49 |
| ATOM | 3351 | OH2 | WAT | 177 | 13.539 | 18.383 | 41.458 | 1.00 | 48.62 |
| ATOM | 3354 | OH2 | WAT | 178 | 23.784 | 2.485 | 27.988 | 1.00 | 45.78 |
| ATOM | 3357 | OH2 | WAT | 179 | 21.591 | 1.684 | 27.146 | 1.00 | 43.48 |
| ATOM | 3360 | OH2 | WAT | 180 | 25.442 | 14.545 | 29.919 | 1.00 | 52.10 |
| ATOM | 3363 | OH2 | WAT | 181 | −0.323 | 34.236 | 33.456 | 1.00 | 30.00 |
| ATOM | 3366 | OH2 | WAT | 182 | −1.400 | 35.866 | 35.094 | 1.00 | 64.81 |
| ATOM | 3369 | OH2 | WAT | 183 | 18.717 | 35.915 | 39.911 | 1.00 | 39.40 |
| ATOM | 3372 | OH2 | WAT | 184 | 20.192 | 3.855 | 19.004 | 1.00 | 65.10 |
| ATOM | 3375 | OH2 | WAT | 185 | 13.114 | 16.579 | 18.007 | 1.00 | 20.91 |
| ATOM | 3378 | OH2 | WAT | 186 | 10.278 | 19.050 | 14.953 | 1.00 | 14.02 |
| ATOM | 3381 | OH2 | WAT | 187 | 5.995 | 17.542 | 9.142 | 1.00 | 44.05 |
| ATOM | 3384 | OH2 | WAT | 188 | 23.018 | 11.761 | 9.729 | 1.00 | 55.21 |
| ATOM | 3387 | OH2 | WAT | 189 | 40.655 | 11.538 | 37.730 | 1.00 | 35.06 |
| ATOM | 3390 | OH2 | WAT | 190 | 35.928 | 15.295 | 47.603 | 1.00 | 26.51 |
| ATOM | 3393 | OH2 | WAT | 191 | 34.985 | 24.299 | 56.702 | 1.00 | 44.83 |
| ATOM | 3396 | OH2 | WAT | 192 | 37.788 | 24.278 | 55.462 | 1.00 | 30.04 |
| ATOM | 3399 | OH2 | WAT | 193 | 19.634 | 24.968 | 7.883 | 1.00 | 31.74 |
| ATOM | 3402 | OH2 | WAT | 194 | 22.698 | 35.658 | 8.378 | 1.00 | 43.95 |
| ATOM | 3405 | OH2 | WAT | 195 | 23.806 | 39.273 | 8.938 | 1.00 | 31.47 |
| ATOM | 3408 | OH2 | WAT | 196 | 14.668 | 33.928 | 45.167 | 1.00 | 38.80 |
| ATOM | 3411 | OH2 | WAT | 197 | 0.503 | 26.774 | 31.064 | 1.00 | 26.19 |
| ATOM | 3414 | OH2 | WAT | 198 | −0.884 | 24.774 | 29.206 | 1.00 | 36.18 |
| ATOM | 3417 | OH2 | WAT | 199 | 33.265 | 30.616 | 36.622 | 1.00 | 28.85 |
| ATOM | 3420 | OH2 | WAT | 200 | −1.883 | 38.952 | 34.210 | 1.00 | 39.70 |
| ATOM | 3423 | OH2 | WAT | 201 | 28.509 | 27.847 | 8.232 | 1.00 | 39.08 |
| ATOM | 3426 | OH2 | WAT | 202 | 26.251 | 33.746 | 8.789 | 1.00 | 40.91 |
| ATOM | 3429 | OH2 | WAT | 203 | 36.519 | 5.008 | 47.232 | 1.00 | 21.32 |
| ATOM | 3432 | OH2 | WAT | 204 | 39.295 | 25.319 | 23.262 | 1.00 | 49.87 |
| ATOM | 3435 | OH2 | WAT | 205 | 34.909 | 12.654 | 30.626 | 1.00 | 42.39 |
| ATOM | 3438 | OH2 | WAT | 206 | 9.995 | 33.513 | 6.870 | 1.00 | 33.84 |
| ATOM | 3441 | OH2 | WAT | 207 | 27.652 | 36.150 | 34.703 | 1.00 | 25.20 |
| ATOM | 3444 | OH2 | WAT | 208 | 31.454 | 37.403 | 30.154 | 1.00 | 62.01 |
| ATOM | 3447 | OH2 | WAT | 209 | 3.316 | 20.483 | 17.051 | 1.00 | 37.60 |
| ATOM | 3450 | OH2 | WAT | 210 | 22.529 | 9.513 | 34.500 | 1.00 | 30.89 |
| ATOM | 3453 | OH2 | WAT | 211 | 24.061 | 10.138 | 30.925 | 1.00 | 53.71 |
| ATOM | 3456 | OH2 | WAT | 212 | 26.407 | 11.862 | 29.483 | 1.00 | 58.43 |
| ATOM | 3459 | OH2 | WAT | 213 | 24.309 | 11.890 | 27.391 | 1.00 | 42.53 |
| ATOM | 3462 | OH2 | WAT | 214 | 20.718 | 13.927 | 23.791 | 1.00 | 34.21 |
| ATOM | 3465 | OH2 | WAT | 215 | 33.155 | 11.250 | 49.105 | 1.00 | 29.04 |
| ATOM | 3468 | OH2 | WAT | 216 | 34.161 | 9.623 | 47.264 | 1.00 | 49.06 |
| ATOM | 3471 | OH2 | WAT | 217 | 37.051 | 10.064 | 47.525 | 1.00 | 35.04 |
| ATOM | 3474 | OH2 | WAT | 218 | 13.043 | 19.110 | 34.861 | 1.00 | 45.77 |
| ATOM | 3477 | OH2 | WAT | 219 | 11.468 | 17.875 | 32.713 | 1.00 | 33.57 |
| ATOM | 3480 | OH2 | WAT | 220 | 42.342 | 25.715 | 34.287 | 1.00 | 40.43 |
| ATOM | 3483 | OH2 | WAT | 221 | 31.777 | 29.619 | 34.907 | 1.00 | 41.84 |
| ATOM | 3486 | OH2 | WAT | 222 | 28.273 | 17.528 | 34.026 | 1.00 | 26.18 |
| ATOM | 3489 | OH2 | WAT | 223 | 14.914 | 21.654 | 35.275 | 1.00 | 35.10 |
| ATOM | 3492 | OH2 | WAT | 224 | 14.758 | 21.475 | 37.921 | 1.00 | 34.66 |
| ATOM | 3495 | OH2 | WAT | 225 | 31.865 | 16.492 | 32.505 | 1.00 | 34.93 |
| ATOM | 3498 | OH2 | WAT | 226 | 25.802 | 17.629 | 34.984 | 1.00 | 36.93 |

TABLE 5
Atomic Coordinates for
VEGFR2KD: 2-Isopropyl-5-methyl-2H-pyrazole-3-carboxylic acid(2-methyl-5-{1-[3-
((E)-2-pyridin-2-yl-vinyl)-1H-indazol-6-yl]-methanoyl}-phenyl)-amide (Compound 5)
Complex Crystalline Structure

| | | | | | | | | | |
|---|---|---|---|---|---|---|---|---|---|
| ATOM | 1 | CB | ASP | 814 | 32.792 | 17.904 | 27.786 | 1.00 | 34.19 |
| ATOM | 2 | CG | ASP | 814 | 31.890 | 17.697 | 26.555 | 1.00 | 31.34 |
| ATOM | 3 | OD1 | ASP | 814 | 30.842 | 18.313 | 26.561 | 1.00 | 34.05 |
| ATOM | 4 | OD2 | ASP | 814 | 32.173 | 16.953 | 25.642 | 1.00 | 29.39 |
| ATOM | 5 | C | ASP | 814 | 34.045 | 15.711 | 27.928 | 1.00 | 38.24 |
| ATOM | 6 | O | ASP | 814 | 33.267 | 15.218 | 28.735 | 1.00 | 35.76 |
| ATOM | 7 | HT1 | ASP | 814 | 34.857 | 17.538 | 25.812 | 1.00 | 0.00 |
| ATOM | 8 | HT2 | ASP | 814 | 36.133 | 17.723 | 26.954 | 1.00 | 0.00 |
| ATOM | 9 | N | ASP | 814 | 35.120 | 17.834 | 26.778 | 1.00 | 38.48 |
| ATOM | 10 | HT3 | ASP | 814 | 34.957 | 18.874 | 26.817 | 1.00 | 0.00 |
| ATOM | 11 | CA | ASP | 814 | 34.215 | 17.247 | 27.846 | 1.00 | 36.49 |
| ATOM | 12 | N | GLU | 815 | 34.742 | 14.937 | 27.050 | 1.00 | 39.62 |
| ATOM | 13 | H | GLU | 815 | 35.232 | 15.324 | 26.306 | 1.00 | 0.00 |
| ATOM | 14 | CA | GLU | 815 | 34.645 | 13.440 | 27.084 | 1.00 | 41.72 |
| ATOM | 15 | CB | GLU | 815 | 34.943 | 12.726 | 25.771 | 1.00 | 47.91 |
| ATOM | 16 | CG | GLU | 815 | 33.745 | 12.754 | 24.767 | 1.00 | 55.41 |
| ATOM | 17 | CD | GLU | 815 | 34.190 | 12.536 | 23.302 | 1.00 | 60.84 |
| ATOM | 18 | OE1 | GLU | 815 | 33.274 | 12.381 | 22.456 | 1.00 | 64.13 |
| ATOM | 19 | OE2 | GLU | 815 | 35.419 | 12.536 | 23.025 | 1.00 | 64.11 |
| ATOM | 20 | C | GLU | 815 | 35.673 | 12.835 | 27.999 | 1.00 | 38.61 |
| ATOM | 21 | O | GLU | 815 | 35.474 | 11.747 | 28.534 | 1.00 | 38.86 |
| ATOM | 22 | N | HIS | 816 | 36.825 | 13.496 | 28.050 | 1.00 | 32.91 |
| ATOM | 23 | H | HIS | 816 | 37.002 | 14.213 | 27.410 | 1.00 | 0.00 |
| ATOM | 24 | CA | HIS | 816 | 37.899 | 13.008 | 28.916 | 1.00 | 30.05 |
| ATOM | 25 | C | HIS | 816 | 38.171 | 14.015 | 30.026 | 1.00 | 27.77 |
| ATOM | 26 | O | HIS | 816 | 39.242 | 14.568 | 30.196 | 1.00 | 26.11 |
| ATOM | 27 | CB | HIS | 816 | 39.135 | 12.741 | 28.008 | 1.00 | 28.88 |
| ATOM | 28 | CG | HIS | 816 | 38.886 | 11.687 | 26.992 | 1.00 | 31.79 |
| ATOM | 29 | ND1 | HIS | 816 | 39.568 | 10.507 | 26.964 | 1.00 | 33.32 |
| ATOM | 30 | CE1 | HIS | 816 | 39.064 | 9.771 | 25.900 | 1.00 | 31.90 |
| ATOM | 31 | CD2 | HIS | 816 | 37.958 | 11.651 | 25.919 | 1.00 | 31.49 |
| ATOM | 32 | NE2 | HIS | 816 | 38.076 | 10.475 | 25.260 | 1.00 | 32.16 |
| ATOM | 33 | HE2 | HIS | 816 | 37.655 | 10.204 | 24.424 | 1.00 | 0.00 |
| ATOM | 34 | N | CYS | 817 | 37.164 | 14.306 | 30.834 | 1.00 | 25.42 |
| ATOM | 35 | H | CYS | 817 | 36.321 | 13.790 | 30.722 | 1.00 | 0.00 |
| ATOM | 36 | CA | CYS | 817 | 37.308 | 15.307 | 31.947 | 1.00 | 23.71 |
| ATOM | 37 | CB | CYS | 817 | 35.956 | 15.620 | 32.666 | 1.00 | 23.28 |
| ATOM | 38 | SG | CYS | 817 | 34.686 | 16.058 | 31.489 | 1.00 | 26.10 |
| ATOM | 39 | C | CYS | 817 | 38.256 | 15.018 | 33.098 | 1.00 | 21.95 |
| ATOM | 40 | O | CYS | 817 | 38.714 | 15.926 | 33.760 | 1.00 | 21.78 |
| ATOM | 41 | N | GLU | 818 | 38.478 | 13.742 | 33.404 | 1.00 | 22.35 |
| ATOM | 42 | H | GLU | 818 | 38.028 | 13.109 | 32.820 | 1.00 | 0.00 |
| ATOM | 43 | CA | GLU | 818 | 39.361 | 13.303 | 34.525 | 1.00 | 21.52 |
| ATOM | 44 | CB | GLU | 818 | 39.422 | 11.768 | 34.522 | 1.00 | 20.64 |
| ATOM | 45 | CG | GLU | 818 | 38.057 | 11.107 | 34.645 | 1.00 | 19.04 |
| ATOM | 46 | CD | GLU | 818 | 37.326 | 10.926 | 33.322 | 1.00 | 22.56 |
| ATOM | 47 | OE1 | GLU | 818 | 36.148 | 10.595 | 33.364 | 1.00 | 20.76 |
| ATOM | 48 | OE2 | GLU | 818 | 37.918 | 11.081 | 32.256 | 1.00 | 23.23 |
| ATOM | 49 | C | GLU | 818 | 40.772 | 13.872 | 34.549 | 1.00 | 22.45 |
| ATOM | 50 | O | GLU | 818 | 41.373 | 14.013 | 35.615 | 1.00 | 23.59 |
| ATOM | 51 | N | ARG | 819 | 41.297 | 14.209 | 33.364 | 1.00 | 21.54 |
| ATOM | 52 | H | ARG | 819 | 40.775 | 13.958 | 32.575 | 1.00 | 0.00 |
| ATOM | 53 | CA | ARG | 819 | 42.656 | 14.791 | 33.233 | 1.00 | 23.95 |
| ATOM | 54 | CB | ARG | 819 | 43.138 | 14.508 | 31.841 | 1.00 | 30.00 |
| ATOM | 55 | CG | ARG | 819 | 42.016 | 15.104 | 30.917 | 1.00 | 38.15 |
| ATOM | 56 | CD | ARG | 819 | 42.247 | 15.163 | 29.434 | 1.00 | 43.11 |
| ATOM | 57 | NE | ARG | 819 | 43.631 | 15.592 | 29.423 | 1.00 | 50.62 |
| ATOM | 58 | HE | ARG | 819 | 43.947 | 16.295 | 30.029 | 1.00 | 0.00 |
| ATOM | 59 | CZ | ARG | 819 | 44.450 | 15.099 | 28.529 | 1.00 | 54.81 |
| ATOM | 60 | NH1 | ARG | 819 | 44.015 | 14.233 | 27.582 | 1.00 | 57.46 |
| ATOM | 61 | HH11 | ARG | 819 | 43.054 | 13.951 | 27.548 | 1.00 | 0.00 |
| ATOM | 62 | HH12 | ARG | 819 | 44.655 | 13.873 | 26.905 | 1.00 | 0.00 |
| ATOM | 63 | NH2 | ARG | 819 | 45.748 | 15.367 | 28.734 | 1.00 | 57.06 |
| ATOM | 64 | HH21 | ARG | 819 | 46.025 | 15.905 | 29.529 | 1.00 | 0.00 |
| ATOM | 65 | HH22 | ARG | 819 | 46.428 | 15.023 | 28.087 | 1.00 | 0.00 |
| ATOM | 66 | C | ARG | 819 | 42.752 | 16.304 | 33.489 | 1.00 | 22.28 |
| ATOM | 67 | O | ARG | 819 | 43.794 | 16.950 | 33.507 | 1.00 | 22.47 |
| ATOM | 68 | N | LEU | 820 | 41.607 | 16.912 | 33.521 | 1.00 | 20.20 |
| ATOM | 69 | H | LEU | 820 | 40.815 | 16.347 | 33.547 | 1.00 | 0.00 |
| ATOM | 70 | CA | LEU | 820 | 41.466 | 18.354 | 33.736 | 1.00 | 18.59 |
| ATOM | 71 | CB | LEU | 820 | 39.969 | 18.654 | 33.393 | 1.00 | 18.69 |
| ATOM | 72 | CG | LEU | 820 | 39.631 | 19.448 | 32.124 | 1.00 | 17.56 |
| ATOM | 73 | CD1 | LEU | 820 | 40.667 | 19.234 | 31.020 | 1.00 | 17.93 |
| ATOM | 74 | CD2 | LEU | 820 | 38.197 | 19.082 | 31.757 | 1.00 | 16.07 |
| ATOM | 75 | C | LEU | 820 | 41.891 | 18.772 | 35.166 | 1.00 | 17.06 |
| ATOM | 76 | O | LEU | 820 | 41.773 | 17.988 | 36.105 | 1.00 | 20.02 |
| ATOM | 77 | N | PRO | 821 | 42.365 | 20.013 | 35.390 | 1.00 | 14.81 |
| ATOM | 78 | CD | PRO | 821 | 42.666 | 21.090 | 34.392 | 1.00 | 12.71 |
| ATOM | 79 | CA | PRO | 821 | 42.840 | 20.403 | 36.704 | 1.00 | 13.37 |
| ATOM | 80 | CB | PRO | 821 | 43.863 | 21.515 | 36.426 | 1.00 | 15.11 |
| ATOM | 81 | CG | PRO | 821 | 43.235 | 22.250 | 35.230 | 1.00 | 11.18 |
| ATOM | 82 | C | PRO | 821 | 41.770 | 20.881 | 37.582 | 1.00 | 13.43 |
| ATOM | 83 | O | PRO | 821 | 40.749 | 21.441 | 37.174 | 1.00 | 13.93 |
| ATOM | 84 | N | TYR | 822 | 42.078 | 20.742 | 38.817 | 1.00 | 14.23 |
| ATOM | 85 | H | TYR | 822 | 42.819 | 20.139 | 39.033 | 1.00 | 0.00 |
| ATOM | 86 | CA | TYR | 822 | 41.202 | 21.258 | 39.883 | 1.00 | 15.62 |
| ATOM | 87 | CB | TYR | 822 | 40.508 | 20.165 | 40.830 | 1.00 | 12.04 |
| ATOM | 88 | CG | TYR | 822 | 39.793 | 20.919 | 41.910 | 1.00 | 13.03 |
| ATOM | 89 | CD1 | TYR | 822 | 40.199 | 20.836 | 43.234 | 1.00 | 13.84 |
| ATOM | 90 | CE1 | TYR | 822 | 39.526 | 21.665 | 44.142 | 1.00 | 18.12 |
| ATOM | 91 | CD2 | TYR | 822 | 38.739 | 21.796 | 41.560 | 1.00 | 14.24 |
| ATOM | 92 | CE2 | TYR | 822 | 38.073 | 22.618 | 42.473 | 1.00 | 14.86 |
| ATOM | 93 | CZ | TYR | 822 | 38.482 | 22.547 | 43.775 | 1.00 | 15.14 |
| ATOM | 94 | OH | TYR | 822 | 37.870 | 23.378 | 44.689 | 1.00 | 14.50 |
| ATOM | 95 | HH | TYR | 822 | 37.116 | 23.819 | 44.285 | 1.00 | 0.00 |
| ATOM | 96 | C | TYR | 822 | 42.111 | 22.118 | 40.782 | 1.00 | 17.73 |
| ATOM | 97 | O | TYR | 822 | 42.834 | 21.632 | 41.672 | 1.00 | 18.94 |
| ATOM | 98 | N | ASP | 823 | 42.150 | 23.415 | 40.493 | 1.00 | 18.01 |
| ATOM | 99 | H | ASP | 823 | 41.581 | 23.742 | 39.765 | 1.00 | 0.00 |
| ATOM | 100 | CA | ASP | 823 | 42.998 | 24.374 | 41.282 | 1.00 | 18.47 |
| ATOM | 101 | CB | ASP | 823 | 43.336 | 25.574 | 40.386 | 1.00 | 20.70 |
| ATOM | 102 | CG | ASP | 823 | 44.171 | 26.651 | 41.093 | 1.00 | 25.60 |
| ATOM | 103 | OD1 | ASP | 823 | 44.548 | 26.465 | 42.260 | 1.00 | 27.52 |
| ATOM | 104 | OD2 | ASP | 823 | 44.448 | 27.682 | 40.456 | 1.00 | 27.59 |
| ATOM | 105 | C | ASP | 823 | 42.305 | 24.835 | 42.568 | 1.00 | 16.18 |
| ATOM | 106 | O | ASP | 823 | 41.642 | 25.863 | 42.660 | 1.00 | 14.86 |
| ATOM | 107 | N | ALA | 824 | 42.434 | 23.996 | 43.573 | 1.00 | 15.80 |
| ATOM | 108 | H | ALA | 824 | 42.883 | 23.143 | 43.377 | 1.00 | 0.00 |
| ATOM | 109 | CA | ALA | 824 | 41.822 | 24.198 | 44.907 | 1.00 | 16.25 |
| ATOM | 110 | CB | ALA | 824 | 42.274 | 23.060 | 45.897 | 1.00 | 15.30 |
| ATOM | 111 | C | ALA | 824 | 42.191 | 25.553 | 45.496 | 1.00 | 16.54 |
| ATOM | 112 | O | ALA | 824 | 41.328 | 26.202 | 46.092 | 1.00 | 13.68 |
| ATOM | 113 | N | SER | 825 | 43.425 | 26.045 | 45.287 | 1.00 | 16.15 |
| ATOM | 114 | H | SER | 825 | 44.091 | 25.534 | 44.796 | 1.00 | 0.00 |
| ATOM | 115 | CA | SER | 825 | 43.714 | 27.364 | 45.855 | 1.00 | 19.81 |
| ATOM | 116 | CB | SER | 825 | 45.230 | 27.783 | 45.681 | 1.00 | 21.01 |
| ATOM | 117 | OG | SER | 825 | 45.588 | 28.116 | 44.348 | 1.00 | 26.58 |
| ATOM | 118 | HG | SER | 825 | 46.533 | 28.283 | 44.302 | 1.00 | 0.00 |
| ATOM | 119 | C | SER | 825 | 42.764 | 28.421 | 45.216 | 1.00 | 22.04 |
| ATOM | 120 | O | SER | 825 | 42.318 | 29.362 | 45.898 | 1.00 | 23.79 |
| ATOM | 121 | N | LYS | 826 | 42.395 | 28.301 | 43.921 | 1.00 | 17.92 |
| ATOM | 122 | H | LYS | 826 | 42.733 | 27.560 | 43.381 | 1.00 | 0.00 |
| ATOM | 123 | CA | LYS | 826 | 41.454 | 29.272 | 43.387 | 1.00 | 16.61 |
| ATOM | 124 | CB | LYS | 826 | 41.603 | 29.266 | 41.869 | 1.00 | 15.46 |
| ATOM | 125 | CG | LYS | 826 | 40.455 | 30.038 | 41.205 | 1.00 | 16.75 |
| ATOM | 126 | CD | LYS | 826 | 40.829 | 30.482 | 39.799 | 1.00 | 17.61 |
| ATOM | 127 | CE | LYS | 826 | 39.634 | 31.089 | 39.068 | 1.00 | 18.52 |
| ATOM | 128 | NZ | LYS | 826 | 40.022 | 31.142 | 37.650 | 1.00 | 19.13 |
| ATOM | 129 | HZ1 | LYS | 826 | 40.353 | 30.196 | 37.373 | 1.00 | 0.00 |
| ATOM | 130 | HZ2 | LYS | 826 | 40.792 | 31.830 | 37.520 | 1.00 | 0.00 |
| ATOM | 131 | HZ3 | LYS | 826 | 39.213 | 31.415 | 37.056 | 1.00 | 0.00 |
| ATOM | 132 | C | LYS | 826 | 39.957 | 28.981 | 43.781 | 1.00 | 17.40 |
| ATOM | 133 | O | LYS | 826 | 39.169 | 29.813 | 44.249 | 1.00 | 16.92 |
| ATOM | 134 | N | TRP | 827 | 39.538 | 27.725 | 43.557 | 1.00 | 16.49 |
| ATOM | 135 | H | TRP | 827 | 40.213 | 27.094 | 43.240 | 1.00 | 0.00 |
| ATOM | 136 | CA | TRP | 827 | 38.140 | 27.295 | 43.763 | 1.00 | 14.07 |
| ATOM | 137 | CB | TRP | 827 | 37.822 | 26.194 | 42.756 | 1.00 | 10.82 |
| ATOM | 138 | CG | TRP | 827 | 38.026 | 26.782 | 41.394 | 1.00 | 10.09 |
| ATOM | 139 | CD2 | TRP | 827 | 37.221 | 27.684 | 40.681 | 1.00 | 10.60 |
| ATOM | 140 | CE2 | TRP | 827 | 37.849 | 27.872 | 39.408 | 1.00 | 9.95 |
| ATOM | 141 | CE3 | TRP | 827 | 36.015 | 28.350 | 40.987 | 1.00 | 10.24 |
| ATOM | 142 | CD1 | TRP | 827 | 39.099 | 26.497 | 40.524 | 1.00 | 8.66 |
| ATOM | 143 | NE1 | TRP | 827 | 38.991 | 27.140 | 39.352 | 1.00 | 6.01 |
| ATOM | 144 | HE1 | TRP | 827 | 39.652 | 27.144 | 38.614 | 1.00 | 0.00 |
| ATOM | 145 | CZ2 | TRP | 827 | 37.253 | 28.726 | 38.442 | 1.00 | 13.84 |
| ATOM | 146 | CZ3 | TRP | 827 | 35.463 | 29.193 | 40.007 | 1.00 | 12.76 |
| ATOM | 147 | CH2 | TRP | 827 | 36.051 | 29.402 | 38.724 | 1.00 | 12.28 |
| ATOM | 148 | C | TRP | 827 | 37.603 | 26.851 | 45.076 | 1.00 | 14.96 |
| ATOM | 149 | O | TRP | 827 | 36.392 | 26.963 | 45.307 | 1.00 | 13.52 |
| ATOM | 150 | N | GLU | 828 | 38.463 | 26.275 | 45.904 | 1.00 | 13.08 |
| ATOM | 151 | H | GLU | 828 | 39.424 | 26.402 | 45.763 | 1.00 | 0.00 |
| ATOM | 152 | CA | GLU | 828 | 37.963 | 25.743 | 47.185 | 1.00 | 16.34 |
| ATOM | 153 | CB | GLU | 828 | 39.155 | 25.045 | 47.869 | 1.00 | 15.40 |
| ATOM | 154 | CG | GLU | 828 | 38.714 | 24.059 | 48.963 | 1.00 | 14.44 |
| ATOM | 155 | CD | GLU | 828 | 37.991 | 22.826 | 48.442 | 1.00 | 14.62 |
| ATOM | 156 | OE1 | GLU | 828 | 37.261 | 22.321 | 49.290 | 1.00 | 12.81 |
| ATOM | 157 | OE2 | GLU | 828 | 38.167 | 22.377 | 47.292 | 1.00 | 10.65 |
| ATOM | 158 | C | GLU | 828 | 37.288 | 26.779 | 48.108 | 1.00 | 15.33 |
| ATOM | 159 | O | GLU | 828 | 37.797 | 27.842 | 48.394 | 1.00 | 15.54 |
| ATOM | 160 | N | PHE | 829 | 36.086 | 26.440 | 48.559 | 1.00 | 16.21 |
| ATOM | 161 | H | PHE | 829 | 35.727 | 25.601 | 48.214 | 1.00 | 0.00 |
| ATOM | 162 | CA | PHE | 829 | 35.218 | 27.254 | 49.436 | 1.00 | 14.05 |
| ATOM | 163 | CB | PHE | 829 | 33.849 | 27.557 | 48.665 | 1.00 | 10.99 |
| ATOM | 164 | CG | PHE | 829 | 33.147 | 28.656 | 49.333 | 1.00 | 11.55 |
| ATOM | 165 | CD1 | PHE | 829 | 33.743 | 29.922 | 49.464 | 1.00 | 10.82 |
| ATOM | 166 | CD2 | PHE | 829 | 31.869 | 28.452 | 49.868 | 1.00 | 12.79 |
| ATOM | 167 | CE1 | PHE | 829 | 33.088 | 30.975 | 50.115 | 1.00 | 10.30 |
| ATOM | 168 | CE2 | PHE | 829 | 31.224 | 29.508 | 50.515 | 1.00 | 11.60 |
| ATOM | 169 | CZ | PHE | 829 | 31.822 | 30.755 | 50.639 | 1.00 | 9.05 |
| ATOM | 170 | C | PHE | 829 | 34.952 | 26.518 | 50.765 | 1.00 | 15.43 |
| ATOM | 171 | O | PHE | 829 | 34.617 | 25.337 | 50.781 | 1.00 | 14.65 |
| ATOM | 172 | N | PRO | 830 | 35.126 | 27.158 | 51.949 | 1.00 | 18.23 |
| ATOM | 173 | CD | PRO | 830 | 35.554 | 28.573 | 52.200 | 1.00 | 19.04 |
| ATOM | 174 | CA | PRO | 830 | 34.903 | 26.447 | 53.215 | 1.00 | 17.34 |
| ATOM | 175 | CB | PRO | 830 | 35.435 | 27.436 | 54.240 | 1.00 | 17.75 |
| ATOM | 176 | CG | PRO | 830 | 36.428 | 28.272 | 53.421 | 1.00 | 17.34 |
| ATOM | 177 | C | PRO | 830 | 33.448 | 26.083 | 53.357 | 1.00 | 18.48 |
| ATOM | 178 | O | PRO | 830 | 32.553 | 26.917 | 53.203 | 1.00 | 19.35 |
| ATOM | 179 | N | ARG | 831 | 33.193 | 24.824 | 53.648 | 1.00 | 19.36 |
| ATOM | 180 | H | ARG | 831 | 33.923 | 24.174 | 53.685 | 1.00 | 0.00 |
| ATOM | 181 | CA | ARG | 831 | 31.809 | 24.345 | 53.787 | 1.00 | 22.13 |
| ATOM | 182 | CB | ARG | 831 | 31.841 | 22.802 | 54.127 | 1.00 | 23.97 |
| ATOM | 183 | CG | ARG | 831 | 31.660 | 21.884 | 52.839 | 1.00 | 28.85 |
| ATOM | 184 | CD | ARG | 831 | 31.420 | 20.284 | 52.772 | 1.00 | 25.78 |
| ATOM | 185 | NE | ARG | 831 | 32.750 | 19.812 | 53.046 | 1.00 | 28.74 |
| ATOM | 186 | HE | ARG | 831 | 33.064 | 19.866 | 53.974 | 1.00 | 0.00 |
| ATOM | 187 | CZ | ARG | 831 | 33.547 | 19.288 | 52.163 | 1.00 | 26.25 |
| ATOM | 188 | NH1 | ARG | 831 | 34.705 | 19.047 | 52.680 | 1.00 | 31.26 |
| ATOM | 189 | HH11 | ARG | 831 | 34.875 | 19.205 | 53.653 | 1.00 | 0.00 |
| ATOM | 190 | HH12 | ARG | 831 | 35.439 | 18.725 | 52.098 | 1.00 | 0.00 |
| ATOM | 191 | NH2 | ARG | 831 | 33.250 | 18.942 | 50.926 | 1.00 | 26.75 |
| ATOM | 192 | HH21 | ARG | 831 | 32.327 | 19.101 | 50.585 | 1.00 | 0.00 |
| ATOM | 193 | HH22 | ARG | 831 | 33.939 | 18.537 | 50.321 | 1.00 | 0.00 |
| ATOM | 194 | C | ARG | 831 | 31.046 | 25.141 | 54.831 | 1.00 | 21.45 |
| ATOM | 195 | O | ARG | 831 | 29.844 | 25.361 | 54.776 | 1.00 | 20.58 |
| ATOM | 196 | N | ASP | 832 | 31.759 | 25.562 | 55.850 | 1.00 | 22.79 |
| ATOM | 197 | H | ASP | 832 | 32.670 | 25.218 | 55.943 | 1.00 | 0.00 |
| ATOM | 198 | CA | ASP | 832 | 31.144 | 26.363 | 56.926 | 1.00 | 24.66 |
| ATOM | 199 | CB | ASP | 832 | 32.038 | 26.342 | 58.176 | 1.00 | 26.69 |
| ATOM | 200 | CG | ASP | 832 | 33.412 | 26.886 | 57.862 | 1.00 | 30.49 |
| ATOM | 201 | OD1 | ASP | 832 | 34.154 | 26.265 | 57.067 | 1.00 | 37.07 |
| ATOM | 202 | OD2 | ASP | 832 | 33.714 | 27.913 | 58.462 | 1.00 | 27.78 |
| ATOM | 203 | C | ASP | 832 | 30.864 | 27.833 | 56.529 | 1.00 | 24.35 |
| ATOM | 204 | O | ASP | 832 | 30.483 | 28.647 | 57.369 | 1.00 | 24.15 |
| ATOM | 205 | N | ARG | 833 | 31.253 | 28.282 | 55.333 | 1.00 | 20.11 |
| ATOM | 206 | H | ARG | 833 | 31.731 | 27.704 | 54.711 | 1.00 | 0.00 |
| ATOM | 207 | CA | ARG | 833 | 30.903 | 29.642 | 54.935 | 1.00 | 16.17 |
| ATOM | 208 | CB | ARG | 833 | 32.089 | 30.230 | 54.191 | 1.00 | 14.95 |
| ATOM | 209 | CG | ARG | 833 | 33.204 | 30.379 | 55.165 | 1.00 | 16.15 |
| ATOM | 210 | CD | ARG | 833 | 33.538 | 31.781 | 54.874 | 1.00 | 20.84 |
| ATOM | 211 | NE | ARG | 833 | 34.805 | 31.993 | 54.146 | 1.00 | 27.78 |
| ATOM | 212 | HE | ARG | 833 | 34.791 | 32.348 | 53.230 | 1.00 | 0.00 |
| ATOM | 213 | CZ | ARG | 833 | 35.989 | 31.723 | 54.732 | 1.00 | 27.36 |
| ATOM | 214 | NH1 | ARG | 833 | 37.130 | 31.948 | 54.127 | 1.00 | 25.41 |
| ATOM | 215 | HH11 | ARG | 833 | 37.146 | 32.330 | 53.202 | 1.00 | 0.00 |
| ATOM | 216 | HH12 | ARG | 833 | 37.988 | 31.728 | 54.589 | 1.00 | 0.00 |
| ATOM | 217 | NH2 | ARG | 833 | 36.073 | 31.154 | 55.918 | 1.00 | 28.20 |
| ATOM | 218 | HH21 | ARG | 833 | 35.241 | 30.912 | 56.421 | 1.00 | 0.00 |
| ATOM | 219 | HH22 | ARG | 833 | 36.968 | 30.957 | 56.319 | 1.00 | 0.00 |
| ATOM | 220 | C | ARG | 833 | 29.642 | 29.544 | 54.064 | 1.00 | 14.55 |
| ATOM | 221 | O | ARG | 833 | 29.170 | 30.471 | 53.409 | 1.00 | 11.52 |
| ATOM | 222 | N | LEU | 834 | 29.192 | 28.303 | 53.899 | 1.00 | 14.66 |
| ATOM | 223 | H | LEU | 834 | 29.664 | 27.557 | 54.322 | 1.00 | 0.00 |
| ATOM | 224 | CA | LEU | 834 | 27.997 | 27.969 | 53.149 | 1.00 | 15.53 |
| ATOM | 225 | CB | LEU | 834 | 28.444 | 26.857 | 52.205 | 1.00 | 16.57 |
| ATOM | 226 | CG | LEU | 834 | 27.574 | 26.497 | 51.011 | 1.00 | 17.21 |
| ATOM | 227 | CD1 | LEU | 834 | 27.489 | 27.672 | 50.001 | 1.00 | 15.90 |
| ATOM | 228 | CD2 | LEU | 834 | 28.193 | 25.239 | 50.348 | 1.00 | 16.08 |
| ATOM | 229 | C | LEU | 834 | 26.804 | 27.554 | 54.071 | 1.00 | 18.19 |
| ATOM | 230 | O | LEU | 834 | 26.869 | 26.599 | 54.854 | 1.00 | 20.08 |
| ATOM | 231 | N | LYS | 835 | 25.731 | 28.339 | 54.096 | 1.00 | 18.80 |
| ATOM | 232 | H | LYS | 835 | 25.791 | 29.211 | 53.645 | 1.00 | 0.00 |
| ATOM | 233 | CA | LYS | 835 | 24.486 | 28.006 | 54.846 | 1.00 | 18.93 |
| ATOM | 234 | CB | LYS | 835 | 23.832 | 29.237 | 55.533 | 1.00 | 18.73 |
| ATOM | 235 | C | LYS | 835 | 23.421 | 27.418 | 53.876 | 1.00 | 18.08 |
| ATOM | 236 | O | LYS | 835 | 22.738 | 28.109 | 53.123 | 1.00 | 17.66 |
| ATOM | 237 | N | LEU | 836 | 23.323 | 26.097 | 53.798 | 1.00 | 17.29 |
| ATOM | 238 | H | LEU | 836 | 24.005 | 25.583 | 54.268 | 1.00 | 0.00 |
| ATOM | 239 | CA | LEU | 836 | 22.369 | 25.397 | 52.956 | 1.00 | 16.65 |
| ATOM | 240 | CB | LEU | 836 | 22.705 | 23.919 | 53.018 | 1.00 | 17.58 |
| ATOM | 241 | CG | LEU | 836 | 23.722 | 23.441 | 51.995 | 1.00 | 22.03 |
| ATOM | 242 | CD1 | LEU | 836 | 25.106 | 23.997 | 52.271 | 1.00 | 25.61 |
| ATOM | 243 | CD2 | LEU | 836 | 23.777 | 21.900 | 52.091 | 1.00 | 27.26 |
| ATOM | 244 | C | LEU | 836 | 20.908 | 25.597 | 53.274 | 1.00 | 17.48 |
| ATOM | 245 | O | LEU | 836 | 20.485 | 25.490 | 54.415 | 1.00 | 19.01 |
| ATOM | 246 | N | GLY | 837 | 20.091 | 25.803 | 52.272 | 1.00 | 16.84 |
| ATOM | 247 | H | GLY | 837 | 20.463 | 25.798 | 51.374 | 1.00 | 0.00 |
| ATOM | 248 | CA | GLY | 837 | 18.662 | 26.014 | 52.452 | 1.00 | 17.98 |
| ATOM | 249 | C | GLY | 837 | 17.796 | 24.952 | 51.815 | 1.00 | 19.55 |
| ATOM | 250 | O | GLY | 837 | 18.032 | 23.744 | 51.854 | 1.00 | 20.70 |
| ATOM | 251 | N | LYS | 838 | 16.774 | 25.360 | 51.132 | 1.00 | 19.89 |
| ATOM | 252 | H | LYS | 838 | 16.669 | 26.315 | 50.997 | 1.00 | 0.00 |
| ATOM | 253 | CA | LYS | 838 | 15.840 | 24.394 | 50.539 | 1.00 | 23.85 |
| ATOM | 254 | CB | LYS | 838 | 14.507 | 25.191 | 50.446 | 1.00 | 29.81 |
| ATOM | 255 | CG | LYS | 838 | 13.197 | 24.479 | 50.992 | 1.00 | 43.45 |
| ATOM | 256 | CD | LYS | 838 | 12.166 | 23.998 | 49.856 | 1.00 | 50.46 |
| ATOM | 257 | CE | LYS | 838 | 10.942 | 23.041 | 50.220 | 1.00 | 54.44 |
| ATOM | 258 | NZ | LYS | 838 | 9.625 | 23.712 | 50.403 | 1.00 | 55.92 |
| ATOM | 259 | HZ1 | LYS | 838 | 9.462 | 24.414 | 49.653 | 1.00 | 0.00 |
| ATOM | 260 | HZ2 | LYS | 838 | 9.627 | 24.194 | 51.325 | 1.00 | 0.00 |
| ATOM | 261 | HZ3 | LYS | 838 | 8.855 | 23.012 | 50.391 | 1.00 | 0.00 |
| ATOM | 262 | C | LYS | 838 | 16.285 | 23.741 | 49.223 | 1.00 | 22.22 |
| ATOM | 263 | O | LYS | 838 | 17.103 | 24.294 | 48.478 | 1.00 | 20.55 |
| ATOM | 264 | N | PRO | 839 | 15.745 | 22.577 | 48.854 | 1.00 | 21.48 |
| ATOM | 265 | CD | PRO | 839 | 14.807 | 21.703 | 49.566 | 1.00 | 23.38 |
| ATOM | 266 | CA | PRO | 839 | 16.092 | 21.989 | 47.550 | 1.00 | 21.24 |
| ATOM | 267 | CB | PRO | 839 | 15.638 | 20.528 | 47.646 | 1.00 | 22.83 |
| ATOM | 268 | CG | PRO | 839 | 14.351 | 20.664 | 48.492 | 1.00 | 23.68 |
| ATOM | 269 | C | PRO | 839 | 15.451 | 22.726 | 46.369 | 1.00 | 19.62 |
| ATOM | 270 | O | PRO | 839 | 14.327 | 23.233 | 46.442 | 1.00 | 21.05 |
| ATOM | 271 | N | LEU | 840 | 16.248 | 22.929 | 45.320 | 1.00 | 17.59 |
| ATOM | 272 | H | LEU | 840 | 17.194 | 22.744 | 45.456 | 1.00 | 0.00 |
| ATOM | 273 | CA | LEU | 840 | 15.802 | 23.569 | 44.058 | 1.00 | 14.70 |
| ATOM | 274 | CB | LEU | 840 | 16.941 | 24.543 | 43.565 | 1.00 | 10.40 |
| ATOM | 275 | CG | LEU | 840 | 17.290 | 25.730 | 44.469 | 1.00 | 7.83 |
| ATOM | 276 | CD1 | LEU | 840 | 18.426 | 26.530 | 43.831 | 1.00 | 4.45 |
| ATOM | 277 | CD2 | LEU | 840 | 16.044 | 26.592 | 44.692 | 1.00 | 7.37 |
| ATOM | 278 | C | LEU | 840 | 15.495 | 22.415 | 43.048 | 1.00 | 15.32 |
| ATOM | 279 | O | LEU | 840 | 14.775 | 22.535 | 42.060 | 1.00 | 16.37 |
| ATOM | 280 | N | GLY | 841 | 16.128 | 21.249 | 43.247 | 1.00 | 15.72 |
| ATOM | 281 | H | GLY | 841 | 16.841 | 21.219 | 43.920 | 1.00 | 0.00 |
| ATOM | 282 | CA | GLY | 841 | 15.865 | 20.059 | 42.423 | 1.00 | 13.49 |
| ATOM | 283 | C | GLY | 841 | 16.456 | 18.774 | 43.026 | 1.00 | 15.33 |
| ATOM | 284 | O | GLY | 841 | 17.524 | 18.770 | 43.638 | 1.00 | 13.64 |
| ATOM | 285 | N | ARG | 842 | 15.776 | 17.648 | 42.875 | 1.00 | 15.48 |
| ATOM | 286 | H | ARG | 842 | 14.946 | 17.670 | 42.360 | 1.00 | 0.00 |
| ATOM | 287 | CA | ARG | 842 | 16.303 | 16.362 | 43.366 | 1.00 | 16.74 |
| ATOM | 288 | CB | ARG | 842 | 15.671 | 15.839 | 44.631 | 1.00 | 20.45 |
| ATOM | 289 | CG | ARG | 842 | 15.346 | 16.933 | 45.576 | 1.00 | 29.34 |
| ATOM | 290 | CD | ARG | 842 | 16.420 | 17.030 | 46.618 | 1.00 | 38.54 |
| ATOM | 291 | NE | ARG | 842 | 16.468 | 15.793 | 47.438 | 1.00 | 46.15 |
| ATOM | 292 | HE | ARG | 842 | 17.172 | 15.141 | 47.243 | 1.00 | 0.00 |
| ATOM | 293 | CZ | ARG | 842 | 15.607 | 15.513 | 48.429 | 1.00 | 48.47 |
| ATOM | 294 | NH1 | ARG | 842 | 14.601 | 16.357 | 48.743 | 1.00 | 50.37 |
| ATOM | 295 | HH11 | ARG | 842 | 14.488 | 17.211 | 48.234 | 1.00 | 0.00 |
| ATOM | 296 | HH12 | ARG | 842 | 13.976 | 16.122 | 49.487 | 1.00 | 0.00 |
| ATOM | 297 | NH2 | ARG | 842 | 15.822 | 14.393 | 49.145 | 1.00 | 48.84 |
| ATOM | 298 | HH21 | ARG | 842 | 16.609 | 13.815 | 48.929 | 1.00 | 0.00 |
| ATOM | 299 | HH22 | ARG | 842 | 15.204 | 14.138 | 49.887 | 1.00 | 0.00 |
| ATOM | 300 | C | ARG | 842 | 15.984 | 15.260 | 42.353 | 1.00 | 17.43 |
| ATOM | 301 | O | ARG | 842 | 14.931 | 15.200 | 41.689 | 1.00 | 16.93 |
| ATOM | 302 | N | GLY | 843 | 16.935 | 14.384 | 42.180 | 1.00 | 13.93 |
| ATOM | 303 | H | GLY | 843 | 17.743 | 14.461 | 42.727 | 1.00 | 0.00 |
| ATOM | 304 | CA | GLY | 843 | 16.777 | 13.266 | 41.272 | 1.00 | 15.31 |
| ATOM | 305 | C | GLY | 843 | 17.008 | 12.023 | 42.136 | 1.00 | 17.79 |
| ATOM | 306 | O | GLY | 843 | 16.809 | 12.065 | 43.362 | 1.00 | 18.61 |
| ATOM | 307 | N | ALA | 844 | 17.399 | 10.919 | 41.515 | 1.00 | 17.40 |
| ATOM | 308 | H | ALA | 844 | 17.558 | 10.953 | 40.550 | 1.00 | 0.00 |
| ATOM | 309 | CA | ALA | 844 | 17.686 | 9.695 | 42.303 | 1.00 | 18.35 |
| ATOM | 310 | CB | ALA | 844 | 17.782 | 8.457 | 41.400 | 1.00 | 17.59 |
| ATOM | 311 | C | ALA | 844 | 18.992 | 9.731 | 43.120 | 1.00 | 16.76 |
| ATOM | 312 | O | ALA | 844 | 19.019 | 9.292 | 44.278 | 1.00 | 17.78 |
| ATOM | 313 | N | PHE | 845 | 20.040 | 10.356 | 42.565 | 1.00 | 14.21 |
| ATOM | 314 | H | PHE | 845 | 19.964 | 10.697 | 41.654 | 1.00 | 0.00 |
| ATOM | 315 | CA | PHE | 845 | 21.364 | 10.363 | 43.245 | 1.00 | 15.00 |
| ATOM | 316 | CB | PHE | 845 | 22.404 | 9.563 | 42.418 | 1.00 | 16.96 |
| ATOM | 317 | CG | PHE | 845 | 21.888 | 8.231 | 42.039 | 1.00 | 16.54 |
| ATOM | 318 | CD1 | PHE | 845 | 21.923 | 7.848 | 40.679 | 1.00 | 19.50 |
| ATOM | 319 | CD2 | PHE | 845 | 21.315 | 7.390 | 42.997 | 1.00 | 21.69 |
| ATOM | 320 | CE1 | PHE | 845 | 21.370 | 6.621 | 40.296 | 1.00 | 20.97 |
| ATOM | 321 | CE2 | PHE | 845 | 20.751 | 6.152 | 42.619 | 1.00 | 23.31 |
| ATOM | 322 | CZ | PHE | 845 | 20.786 | 5.778 | 41.270 | 1.00 | 21.05 |
| ATOM | 323 | C | PHE | 845 | 21.964 | 11.717 | 43.496 | 1.00 | 13.67 |
| ATOM | 324 | O | PHE | 845 | 22.938 | 11.869 | 44.220 | 1.00 | 11.56 |
| ATOM | 325 | N | GLY | 846 | 21.307 | 12.739 | 42.975 | 1.00 | 14.40 |
| ATOM | 326 | H | GLY | 846 | 20.441 | 12.575 | 42.550 | 1.00 | 0.00 |
| ATOM | 327 | CA | GLY | 846 | 21.793 | 14.107 | 43.108 | 1.00 | 11.91 |
| ATOM | 328 | C | GLY | 846 | 20.703 | 15.111 | 43.373 | 1.00 | 10.53 |
| ATOM | 329 | O | GLY | 846 | 19.523 | 14.833 | 43.267 | 1.00 | 9.77 |
| ATOM | 330 | N | GLN | 847 | 21.133 | 16.307 | 43.727 | 1.00 | 9.97 |
| ATOM | 331 | H | GLN | 847 | 22.097 | 16.450 | 43.835 | 1.00 | 0.00 |
| ATOM | 332 | CA | GLN | 847 | 20.248 | 17.415 | 44.032 | 1.00 | 10.90 |
| ATOM | 333 | CB | GLN | 847 | 19.663 | 17.308 | 45.471 | 1.00 | 12.62 |
| ATOM | 334 | CG | GLN | 847 | 20.799 | 17.254 | 46.512 | 1.00 | 12.61 |
| ATOM | 335 | CD | GLN | 847 | 20.369 | 16.839 | 47.881 | 1.00 | 12.10 |
| ATOM | 336 | OE1 | GLN | 847 | 19.251 | 16.904 | 48.323 | 1.00 | 13.50 |
| ATOM | 337 | NE2 | GLN | 847 | 21.240 | 16.324 | 48.654 | 1.00 | 15.65 |
| ATOM | 338 | HE21 | GLN | 847 | 22.171 | 16.186 | 48.388 | 1.00 | 0.00 |
| ATOM | 339 | HE22 | GLN | 847 | 20.906 | 16.075 | 49.530 | 1.00 | 0.00 |
| ATOM | 340 | C | GLN | 847 | 20.986 | 18.728 | 43.957 | 1.00 | 11.32 |
| ATOM | 341 | O | GLN | 847 | 22.227 | 18.817 | 43.982 | 1.00 | 11.52 |
| ATOM | 342 | N | VAL | 848 | 20.166 | 19.738 | 43.714 | 1.00 | 12.63 |
| ATOM | 343 | H | VAL | 848 | 19.221 | 19.544 | 43.558 | 1.00 | 0.00 |
| ATOM | 344 | CA | VAL | 848 | 20.652 | 21.117 | 43.675 | 1.00 | 13.38 |
| ATOM | 345 | CB | VAL | 848 | 20.365 | 21.834 | 42.332 | 1.00 | 12.14 |
| ATOM | 346 | CG1 | VAL | 848 | 20.856 | 23.327 | 42.388 | 1.00 | 13.81 |
| ATOM | 347 | CG2 | VAL | 848 | 21.228 | 21.177 | 41.258 | 1.00 | 8.08 |
| ATOM | 348 | C | VAL | 848 | 19.915 | 21.852 | 44.805 | 1.00 | 13.65 |
| ATOM | 349 | O | VAL | 848 | 18.674 | 21.843 | 44.866 | 1.00 | 15.41 |
| ATOM | 350 | N | ILE | 849 | 20.653 | 22.397 | 45.751 | 1.00 | 12.17 |
| ATOM | 351 | H | ILE | 849 | 21.628 | 22.282 | 45.765 | 1.00 | 0.00 |
| ATOM | 352 | CA | ILE | 849 | 19.936 | 23.111 | 46.813 | 1.00 | 14.26 |
| ATOM | 353 | CB | ILE | 849 | 20.017 | 22.297 | 48.212 | 1.00 | 15.42 |
| ATOM | 354 | CG2 | ILE | 849 | 20.403 | 20.785 | 48.110 | 1.00 | 12.84 |
| ATOM | 355 | CG1 | ILE | 849 | 21.034 | 22.901 | 49.027 | 1.00 | 15.12 |
| ATOM | 356 | CD1 | ILE | 849 | 20.162 | 23.336 | 50.138 | 1.00 | 13.22 |
| ATOM | 357 | C | ILE | 849 | 20.428 | 24.547 | 46.962 | 1.00 | 14.93 |
| ATOM | 358 | O | ILE | 849 | 21.601 | 24.901 | 46.712 | 1.00 | 14.17 |
| ATOM | 359 | N | GLU | 850 | 19.465 | 25.392 | 47.291 | 1.00 | 13.78 |
| ATOM | 360 | H | GLU | 850 | 18.587 | 25.024 | 47.509 | 1.00 | 0.00 |
| ATOM | 361 | CA | GLU | 850 | 19.703 | 26.835 | 47.484 | 1.00 | 15.28 |
| ATOM | 362 | CB | GLU | 850 | 18.396 | 27.544 | 47.763 | 1.00 | 19.32 |
| ATOM | 363 | CG | GLU | 850 | 18.512 | 29.035 | 47.463 | 1.00 | 22.72 |
| ATOM | 364 | CD | GLU | 850 | 17.171 | 29.753 | 47.572 | 1.00 | 26.00 |
| ATOM | 365 | OE1 | GLU | 850 | 16.096 | 29.244 | 47.203 | 1.00 | 27.20 |
| ATOM | 366 | OE2 | GLU | 850 | 17.229 | 30.888 | 48.024 | 1.00 | 32.08 |
| ATOM | 367 | C | GLU | 850 | 20.574 | 27.069 | 48.663 | 1.00 | 13.48 |
| ATOM | 368 | O | GLU | 850 | 20.408 | 26.325 | 49.626 | 1.00 | 13.55 |
| ATOM | 369 | N | ALA | 851 | 21.441 | 28.070 | 48.635 | 1.00 | 11.86 |
| ATOM | 370 | H | ALA | 851 | 21.528 | 28.593 | 47.816 | 1.00 | 0.00 |
| ATOM | 371 | CA | ALA | 851 | 22.269 | 28.328 | 49.801 | 1.00 | 12.12 |
| ATOM | 372 | CB | ALA | 851 | 23.582 | 27.512 | 49.826 | 1.00 | 8.80 |
| ATOM | 373 | C | ALA | 851 | 22.661 | 29.757 | 49.891 | 1.00 | 13.21 |
| ATOM | 374 | O | ALA | 851 | 22.384 | 30.561 | 49.016 | 1.00 | 14.66 |
| ATOM | 375 | N | ASP | 852 | 23.210 | 30.152 | 51.011 | 1.00 | 15.76 |
| ATOM | 376 | H | ASP | 852 | 23.255 | 29.543 | 51.775 | 1.00 | 0.00 |
| ATOM | 377 | CA | ASP | 852 | 23.636 | 31.532 | 51.154 | 1.00 | 17.73 |
| ATOM | 378 | CB | ASP | 852 | 22.583 | 32.143 | 52.148 | 1.00 | 23.56 |
| ATOM | 379 | CG | ASP | 852 | 21.234 | 32.367 | 51.262 | 1.00 | 37.72 |
| ATOM | 380 | OD1 | ASP | 852 | 20.414 | 31.405 | 51.048 | 1.00 | 41.02 |
| ATOM | 381 | OD2 | ASP | 852 | 21.038 | 33.513 | 50.744 | 1.00 | 37.09 |
| ATOM | 382 | C | ASP | 852 | 25.070 | 31.462 | 51.532 | 1.00 | 15.88 |
| ATOM | 383 | O | ASP | 852 | 25.485 | 30.846 | 52.514 | 1.00 | 17.76 |
| ATOM | 384 | N | ALA | 853 | 25.888 | 31.760 | 50.544 | 1.00 | 14.45 |
| ATOM | 385 | H | ALA | 853 | 25.485 | 32.011 | 49.691 | 1.00 | 0.00 |
| ATOM | 386 | CA | ALA | 853 | 27.352 | 31.734 | 50.686 | 1.00 | 13.68 |
| ATOM | 387 | CB | ALA | 853 | 28.061 | 31.360 | 49.351 | 1.00 | 10.34 |
| ATOM | 388 | C | ALA | 853 | 27.996 | 33.046 | 51.141 | 1.00 | 15.34 |
| ATOM | 389 | O | ALA | 853 | 27.820 | 34.085 | 50.488 | 1.00 | 17.75 |
| ATOM | 390 | N | PHE | 854 | 28.755 | 33.050 | 52.236 | 1.00 | 12.76 |
| ATOM | 391 | H | PHE | 854 | 28.883 | 32.196 | 52.698 | 1.00 | 0.00 |
| ATOM | 392 | CA | PHE | 854 | 29.401 | 34.239 | 52.730 | 1.00 | 11.10 |
| ATOM | 393 | CB | PHE | 854 | 29.631 | 34.095 | 54.243 | 1.00 | 11.64 |
| ATOM | 394 | CG | PHE | 854 | 30.028 | 35.424 | 54.752 | 1.00 | 13.57 |
| ATOM | 395 | CD1 | PHE | 854 | 31.377 | 35.731 | 55.041 | 1.00 | 17.21 |
| ATOM | 396 | CD2 | PHE | 854 | 29.043 | 36.392 | 54.982 | 1.00 | 14.89 |
| ATOM | 397 | CE1 | PHE | 854 | 31.755 | 36.986 | 55.562 | 1.00 | 17.08 |
| ATOM | 398 | CE2 | PHE | 854 | 29.406 | 37.641 | 55.498 | 1.00 | 14.87 |
| ATOM | 399 | CZ | PHE | 854 | 30.741 | 37.935 | 55.788 | 1.00 | 14.88 |
| ATOM | 400 | C | PHE | 854 | 30.700 | 34.576 | 52.030 | 1.00 | 10.52 |
| ATOM | 401 | O | PHE | 854 | 31.728 | 33.911 | 52.162 | 1.00 | 10.77 |
| ATOM | 402 | N | GLY | 855 | 30.696 | 35.638 | 51.259 | 1.00 | 10.04 |
| ATOM | 403 | H | GLY | 855 | 29.856 | 36.127 | 51.161 | 1.00 | 0.00 |
| ATOM | 404 | CA | GLY | 855 | 31.917 | 36.067 | 50.565 | 1.00 | 13.07 |
| ATOM | 405 | C | GLY | 855 | 32.471 | 35.179 | 49.464 | 1.00 | 14.16 |
| ATOM | 406 | O | GLY | 855 | 33.677 | 35.205 | 49.206 | 1.00 | 15.80 |
| ATOM | 407 | N | ILE | 856 | 31.626 | 34.428 | 48.764 | 1.00 | 15.66 |
| ATOM | 408 | H | ILE | 856 | 30.746 | 34.261 | 49.143 | 1.00 | 0.00 |
| ATOM | 409 | CA | ILE | 856 | 32.123 | 33.566 | 47.639 | 1.00 | 15.67 |
| ATOM | 410 | CB | ILE | 856 | 30.961 | 32.543 | 47.180 | 1.00 | 15.47 |
| ATOM | 411 | CG2 | ILE | 856 | 29.772 | 33.170 | 46.364 | 1.00 | 10.66 |
| ATOM | 412 | CG1 | ILE | 856 | 31.749 | 31.392 | 46.430 | 1.00 | 14.48 |
| ATOM | 413 | CD1 | ILE | 856 | 30.901 | 30.100 | 46.336 | 1.00 | 11.81 |
| ATOM | 414 | C | ILE | 856 | 32.643 | 34.375 | 46.443 | 1.00 | 16.95 |
| ATOM | 415 | O | ILE | 856 | 33.679 | 34.024 | 45.916 | 1.00 | 18.94 |
| ATOM | 416 | N | ASP | 857 | 31.988 | 35.435 | 45.956 | 1.00 | 17.76 |
| ATOM | 417 | H | ASP | 857 | 31.130 | 35.684 | 46.356 | 1.00 | 0.00 |
| ATOM | 418 | CA | ASP | 857 | 32.471 | 36.205 | 44.800 | 1.00 | 22.15 |
| ATOM | 419 | CB | ASP | 857 | 31.416 | 36.077 | 43.634 | 1.00 | 22.91 |
| ATOM | 420 | CG | ASP | 857 | 29.972 | 36.567 | 43.842 | 1.00 | 28.46 |
| ATOM | 421 | OD1 | ASP | 857 | 29.653 | 37.229 | 44.843 | 1.00 | 30.58 |
| ATOM | 422 | OD2 | ASP | 857 | 29.137 | 36.271 | 42.967 | 1.00 | 28.52 |
| ATOM | 423 | C | ASP | 857 | 32.823 | 37.701 | 45.065 | 1.00 | 23.50 |
| ATOM | 424 | O | ASP | 857 | 33.346 | 38.433 | 44.233 | 1.00 | 26.74 |
| ATOM | 425 | N | LYS | 858 | 32.536 | 38.189 | 46.266 | 1.00 | 25.16 |
| ATOM | 426 | H | LYS | 858 | 31.989 | 37.632 | 46.863 | 1.00 | 0.00 |
| ATOM | 427 | CA | LYS | 858 | 32.819 | 39.574 | 46.700 | 1.00 | 22.69 |
| ATOM | 428 | CB | LYS | 858 | 31.640 | 40.451 | 46.284 | 1.00 | 26.17 |
| ATOM | 429 | CG | LYS | 858 | 31.851 | 41.982 | 46.208 | 1.00 | 29.21 |
| ATOM | 430 | CD | LYS | 858 | 30.461 | 42.663 | 45.970 | 1.00 | 29.77 |
| ATOM | 431 | CE | LYS | 858 | 30.508 | 44.123 | 46.442 | 1.00 | 30.78 |
| ATOM | 432 | NZ | LYS | 858 | 29.148 | 44.686 | 46.369 | 1.00 | 35.10 |
| ATOM | 433 | HZ1 | LYS | 858 | 28.557 | 44.043 | 46.930 | 1.00 | 0.00 |
| ATOM | 434 | HZ2 | LYS | 858 | 28.796 | 44.727 | 45.393 | 1.00 | 0.00 |
| ATOM | 435 | HZ3 | LYS | 858 | 29.135 | 45.634 | 46.801 | 1.00 | 0.00 |
| ATOM | 436 | C | LYS | 858 | 33.000 | 39.556 | 48.218 | 1.00 | 19.72 |
| ATOM | 437 | O | LYS | 858 | 32.219 | 39.020 | 49.020 | 1.00 | 14.81 |
| ATOM | 438 | N | THR | 859 | 34.147 | 40.067 | 48.592 | 1.00 | 18.54 |
| ATOM | 439 | H | THR | 859 | 34.810 | 40.335 | 47.915 | 1.00 | 0.00 |
| ATOM | 440 | CA | THR | 859 | 34.518 | 40.087 | 50.006 | 1.00 | 20.31 |
| ATOM | 441 | CB | THR | 859 | 35.717 | 41.058 | 50.289 | 1.00 | 23.50 |
| ATOM | 442 | OG1 | THR | 859 | 36.785 | 40.568 | 49.455 | 1.00 | 26.68 |
| ATOM | 443 | HG1 | THR | 859 | 37.587 | 41.069 | 49.635 | 1.00 | 0.00 |
| ATOM | 444 | CG2 | THR | 859 | 36.231 | 41.102 | 51.784 | 1.00 | 21.86 |
| ATOM | 445 | C | THR | 859 | 33.437 | 40.472 | 50.960 | 1.00 | 18.91 |
| ATOM | 446 | O | THR | 859 | 32.765 | 41.471 | 50.793 | 1.00 | 18.82 |
| ATOM | 447 | N | ALA | 860 | 33.310 | 39.675 | 51.999 | 1.00 | 17.61 |
| ATOM | 448 | H | ALA | 860 | 33.869 | 38.873 | 51.981 | 1.00 | 0.00 |
| ATOM | 449 | CA | ALA | 860 | 32.363 | 39.866 | 53.106 | 1.00 | 15.98 |
| ATOM | 450 | CB | ALA | 860 | 32.969 | 40.993 | 53.998 | 1.00 | 11.93 |
| ATOM | 451 | C | ALA | 860 | 30.900 | 40.128 | 52.712 | 1.00 | 15.25 |
| ATOM | 452 | O | ALA | 860 | 30.137 | 40.811 | 53.396 | 1.00 | 15.10 |
| ATOM | 453 | N | THR | 861 | 30.440 | 39.550 | 51.594 | 1.00 | 16.85 |
| ATOM | 454 | H | THR | 861 | 31.061 | 39.161 | 50.946 | 1.00 | 0.00 |
| ATOM | 455 | CA | THR | 861 | 29.016 | 39.733 | 51.220 | 1.00 | 16.36 |
| ATOM | 456 | CB | THR | 861 | 28.884 | 40.855 | 50.011 | 1.00 | 17.08 |
| ATOM | 457 | OG1 | THR | 861 | 28.276 | 40.334 | 48.849 | 1.00 | 22.00 |
| ATOM | 458 | HG1 | THR | 861 | 28.005 | 41.045 | 48.250 | 1.00 | 0.00 |
| ATOM | 459 | CG2 | THR | 861 | 30.189 | 41.440 | 49.617 | 1.00 | 11.87 |
| ATOM | 460 | C | THR | 861 | 28.285 | 38.410 | 50.920 | 1.00 | 14.34 |
| ATOM | 461 | O | THR | 861 | 28.873 | 37.471 | 50.353 | 1.00 | 11.67 |
| ATOM | 462 | N | CYS | 862 | 27.087 | 38.228 | 51.566 | 1.00 | 15.84 |
| ATOM | 463 | H | CYS | 862 | 26.699 | 39.027 | 51.972 | 1.00 | 0.00 |
| ATOM | 464 | CA | CYS | 862 | 26.264 | 36.970 | 51.359 | 1.00 | 20.79 |
| ATOM | 465 | CB | CYS | 862 | 24.842 | 36.640 | 52.001 | 1.00 | 25.92 |
| ATOM | 466 | SG | CYS | 862 | 24.173 | 34.802 | 52.280 | 1.00 | 42.81 |
| ATOM | 467 | C | CYS | 862 | 25.770 | 37.010 | 49.959 | 1.00 | 18.96 |
| ATOM | 468 | O | CYS | 862 | 25.475 | 38.058 | 49.383 | 1.00 | 16.49 |
| ATOM | 469 | N | ARG | 863 | 25.649 | 35.799 | 49.463 | 1.00 | 17.65 |
| ATOM | 470 | H | ARG | 863 | 25.901 | 35.030 | 50.022 | 1.00 | 0.00 |
| ATOM | 471 | CA | ARG | 863 | 25.245 | 35.621 | 48.083 | 1.00 | 16.24 |
| ATOM | 472 | CB | ARG | 863 | 26.579 | 35.597 | 47.344 | 1.00 | 17.23 |
| ATOM | 473 | CG | ARG | 863 | 26.682 | 35.004 | 45.966 | 1.00 | 26.00 |
| ATOM | 474 | CD | ARG | 863 | 25.756 | 35.667 | 44.946 | 1.00 | 33.54 |
| ATOM | 475 | NE | ARG | 863 | 26.366 | 35.568 | 43.586 | 1.00 | 40.58 |
| ATOM | 476 | HE | ARG | 863 | 27.258 | 35.945 | 43.519 | 1.00 | 0.00 |
| ATOM | 477 | CZ | ARG | 863 | 25.858 | 34.958 | 42.454 | 1.00 | 43.27 |
| ATOM | 478 | NH1 | ARG | 863 | 26.693 | 35.090 | 41.390 | 1.00 | 40.59 |
| ATOM | 479 | HH11 | ARG | 863 | 27.561 | 35.574 | 41.501 | 1.00 | 0.00 |
| ATOM | 480 | HH12 | ARG | 863 | 26.426 | 34.706 | 40.507 | 1.00 | 0.00 |
| ATOM | 481 | NH2 | ARG | 863 | 24.661 | 34.218 | 42.381 | 1.00 | 37.02 |
| ATOM | 482 | HH21 | ARG | 863 | 24.114 | 34.069 | 43.207 | 1.00 | 0.00 |
| ATOM | 483 | HH22 | ARG | 863 | 24.356 | 33.833 | 41.508 | 1.00 | 0.00 |
| ATOM | 484 | C | ARG | 863 | 24.456 | 34.357 | 48.021 | 1.00 | 14.76 |
| ATOM | 485 | O | ARG | 863 | 24.909 | 33.332 | 48.547 | 1.00 | 13.12 |
| ATOM | 486 | N | THR | 864 | 23.208 | 34.465 | 47.571 | 1.00 | 13.08 |
| ATOM | 487 | H | THR | 864 | 22.791 | 35.355 | 47.502 | 1.00 | 0.00 |
| ATOM | 488 | CA | THR | 864 | 22.398 | 33.247 | 47.381 | 1.00 | 12.56 |
| ATOM | 489 | CB | THR | 864 | 20.956 | 33.491 | 47.028 | 1.00 | 11.92 |
| ATOM | 490 | OG1 | THR | 864 | 20.417 | 34.401 | 47.975 | 1.00 | 16.23 |
| ATOM | 491 | HG1 | THR | 864 | 19.553 | 34.719 | 47.664 | 1.00 | 0.00 |
| ATOM | 492 | CG2 | THR | 864 | 20.198 | 32.151 | 47.019 | 1.00 | 7.87 |
| ATOM | 493 | C | THR | 864 | 22.968 | 32.543 | 46.179 | 1.00 | 12.61 |
| ATOM | 494 | O | THR | 864 | 23.130 | 33.081 | 45.079 | 1.00 | 11.21 |
| ATOM | 495 | N | VAL | 865 | 23.294 | 31.299 | 46.398 | 1.00 | 13.02 |
| ATOM | 496 | H | VAL | 865 | 23.101 | 30.952 | 47.294 | 1.00 | 0.00 |
| ATOM | 497 | CA | VAL | 865 | 23.883 | 30.421 | 45.360 | 1.00 | 12.23 |
| ATOM | 498 | CB | VAL | 865 | 25.427 | 30.104 | 45.705 | 1.00 | 11.27 |
| ATOM | 499 | CG1 | VAL | 865 | 26.233 | 31.371 | 45.710 | 1.00 | 7.29 |
| ATOM | 500 | CG2 | VAL | 865 | 25.540 | 29.348 | 47.055 | 1.00 | 9.31 |
| ATOM | 501 | C | VAL | 865 | 23.083 | 29.080 | 45.184 | 1.00 | 11.12 |
| ATOM | 502 | O | VAL | 865 | 22.162 | 28.806 | 45.958 | 1.00 | 9.32 |
| ATOM | 503 | N | ALA | 866 | 23.397 | 28.291 | 44.126 | 1.00 | 9.71 |
| ATOM | 504 | H | ALA | 866 | 24.114 | 28.613 | 43.534 | 1.00 | 0.00 |
| ATOM | 505 | CA | ALA | 866 | 22.757 | 26.964 | 43.852 | 1.00 | 9.86 |
| ATOM | 506 | CB | ALA | 866 | 22.191 | 26.838 | 42.427 | 1.00 | 3.58 |
| ATOM | 507 | C | ALA | 866 | 23.881 | 25.890 | 43.961 | 1.00 | 10.40 |
| ATOM | 508 | O | ALA | 866 | 24.958 | 26.007 | 43.345 | 1.00 | 11.34 |
| ATOM | 509 | N | VAL | 867 | 23.803 | 24.914 | 44.879 | 1.00 | 11.39 |
| ATOM | 510 | H | VAL | 867 | 23.043 | 24.868 | 45.492 | 1.00 | 0.00 |
| ATOM | 511 | CA | VAL | 867 | 24.899 | 23.910 | 44.942 | 1.00 | 11.20 |
| ATOM | 512 | CB | VAL | 867 | 25.731 | 23.966 | 46.387 | 1.00 | 13.31 |
| ATOM | 513 | CG1 | VAL | 867 | 25.155 | 25.087 | 47.265 | 1.00 | 9.88 |
| ATOM | 514 | CG2 | VAL | 867 | 25.850 | 22.603 | 47.025 | 1.00 | 9.49 |
| ATOM | 515 | C | VAL | 867 | 24.427 | 22.494 | 44.621 | 1.00 | 11.75 |
| ATOM | 516 | O | VAL | 867 | 23.360 | 22.019 | 45.042 | 1.00 | 12.12 |
| ATOM | 517 | N | LYS | 868 | 25.103 | 21.955 | 43.627 | 1.00 | 11.96 |
| ATOM | 518 | H | LYS | 868 | 25.793 | 22.485 | 43.188 | 1.00 | 0.00 |
| ATOM | 519 | CA | LYS | 868 | 24.829 | 20.586 | 43.249 | 1.00 | 10.22 |
| ATOM | 520 | CB | LYS | 868 | 25.108 | 20.360 | 41.760 | 1.00 | 10.01 |
| ATOM | 521 | CG | LYS | 868 | 24.680 | 18.990 | 41.275 | 1.00 | 8.60 |
| ATOM | 522 | CD | LYS | 868 | 24.767 | 18.718 | 39.772 | 1.00 | 8.46 |
| ATOM | 523 | CE | LYS | 868 | 23.832 | 19.521 | 38.883 | 1.00 | 7.23 |
| ATOM | 524 | NZ | LYS | 868 | 24.249 | 19.196 | 37.523 | 1.00 | 2.40 |
| ATOM | 525 | HZ1 | LYS | 868 | 24.311 | 18.164 | 37.432 | 1.00 | 0.00 |
| ATOM | 526 | HZ2 | LYS | 868 | 25.167 | 19.641 | 37.332 | 1.00 | 0.00 |
| ATOM | 527 | HZ3 | LYS | 868 | 23.548 | 19.574 | 36.859 | 1.00 | 0.00 |
| ATOM | 528 | C | LYS | 868 | 25.737 | 19.706 | 44.086 | 1.00 | 8.09 |
| ATOM | 529 | O | LYS | 868 | 26.900 | 19.973 | 44.365 | 1.00 | 6.58 |
| ATOM | 530 | N | MET | 869 | 25.103 | 18.716 | 44.673 | 1.00 | 8.98 |
| ATOM | 531 | H | MET | 869 | 24.136 | 18.662 | 44.540 | 1.00 | 0.00 |
| ATOM | 532 | CA | MET | 869 | 25.751 | 17.694 | 45.508 | 1.00 | 8.78 |
| ATOM | 533 | CB | MET | 869 | 25.858 | 18.161 | 47.012 | 1.00 | 6.48 |
| ATOM | 534 | CG | MET | 869 | 24.477 | 18.368 | 47.629 | 1.00 | 4.78 |
| ATOM | 535 | SD | MET | 869 | 24.505 | 18.947 | 49.342 | 1.00 | 16.24 |
| ATOM | 536 | CE | MET | 869 | 25.087 | 17.411 | 50.175 | 1.00 | 15.26 |
| ATOM | 537 | C | MET | 869 | 24.898 | 16.405 | 45.402 | 1.00 | 9.62 |
| ATOM | 538 | O | MET | 869 | 23.834 | 16.320 | 44.774 | 1.00 | 10.23 |
| ATOM | 539 | N | LEU | 870 | 25.409 | 15.315 | 45.943 | 1.00 | 11.89 |
| ATOM | 540 | H | LEU | 870 | 26.369 | 15.333 | 46.100 | 1.00 | 0.00 |
| ATOM | 541 | CA | LEU | 870 | 24.695 | 14.043 | 45.907 | 1.00 | 11.08 |
| ATOM | 542 | CB | LEU | 870 | 25.809 | 12.955 | 45.753 | 1.00 | 10.34 |
| ATOM | 543 | CG | LEU | 870 | 26.798 | 13.111 | 44.486 | 1.00 | 7.85 |
| ATOM | 544 | CD1 | LEU | 870 | 27.816 | 11.941 | 44.454 | 1.00 | 3.69 |
| ATOM | 545 | CD2 | LEU | 870 | 26.017 | 13.124 | 43.173 | 1.00 | 9.28 |
| ATOM | 546 | C | LEU | 870 | 23.770 | 13.849 | 47.099 | 1.00 | 10.27 |
| ATOM | 547 | O | LEU | 870 | 23.733 | 14.642 | 48.046 | 1.00 | 7.60 |
| ATOM | 548 | N | LYS | 871 | 23.020 | 12.773 | 47.044 | 1.00 | 10.68 |
| ATOM | 549 | H | LYS | 871 | 23.098 | 12.178 | 46.270 | 1.00 | 0.00 |
| ATOM | 550 | CA | LYS | 871 | 22.032 | 12.393 | 48.067 | 1.00 | 11.52 |
| ATOM | 551 | CB | LYS | 871 | 20.599 | 12.814 | 47.681 | 1.00 | 12.77 |
| ATOM | 552 | CG | LYS | 871 | 20.221 | 12.491 | 46.261 | 1.00 | 14.47 |
| ATOM | 553 | CD | LYS | 871 | 18.814 | 12.937 | 46.117 | 1.00 | 20.92 |
| ATOM | 554 | CE | LYS | 871 | 17.869 | 12.181 | 47.033 | 1.00 | 23.93 |
| ATOM | 555 | NZ | LYS | 871 | 17.944 | 10.728 | 46.784 | 1.00 | 27.94 |
| ATOM | 556 | HZ1 | LYS | 871 | 18.748 | 10.360 | 47.329 | 1.00 | 0.00 |
| ATOM | 557 | HZ2 | LYS | 871 | 18.047 | 10.517 | 45.771 | 1.00 | 0.00 |
| ATOM | 558 | HZ3 | LYS | 871 | 17.053 | 10.307 | 47.124 | 1.00 | 0.00 |
| ATOM | 559 | C | LYS | 871 | 22.014 | 10.910 | 48.242 | 1.00 | 10.87 |
| ATOM | 560 | O | LYS | 871 | 22.693 | 10.189 | 47.551 | 1.00 | 11.38 |
| ATOM | 561 | N | GLU | 872 | 21.275 | 10.433 | 49.214 | 1.00 | 12.08 |
| ATOM | 562 | H | GLU | 872 | 20.845 | 11.060 | 49.812 | 1.00 | 0.00 |
| ATOM | 563 | CA | GLU | 872 | 21.136 | 9.005 | 49.477 | 1.00 | 12.03 |
| ATOM | 564 | CB | GLU | 872 | 19.993 | 8.864 | 50.503 | 1.00 | 12.52 |
| ATOM | 565 | CG | GLU | 872 | 19.596 | 7.412 | 50.841 | 1.00 | 23.29 |
| ATOM | 566 | CD | GLU | 872 | 20.769 | 6.524 | 51.442 | 1.00 | 27.30 |
| ATOM | 567 | OE1 | GLU | 872 | 20.756 | 5.319 | 51.116 | 1.00 | 30.11 |
| ATOM | 568 | OE2 | GLU | 872 | 21.644 | 6.998 | 52.221 | 1.00 | 31.07 |
| ATOM | 569 | C | GLU | 872 | 20.853 | 8.240 | 48.189 | 1.00 | 12.13 |
| ATOM | 570 | O | GLU | 872 | 19.972 | 8.613 | 47.406 | 1.00 | 11.81 |
| ATOM | 571 | N | GLY | 873 | 21.605 | 7.157 | 47.944 | 1.00 | 11.44 |
| ATOM | 572 | H | GLY | 873 | 22.385 | 6.991 | 48.510 | 1.00 | 0.00 |
| ATOM | 573 | CA | GLY | 873 | 21.371 | 6.412 | 46.704 | 1.00 | 11.90 |
| ATOM | 574 | C | GLY | 873 | 22.622 | 6.493 | 45.857 | 1.00 | 11.57 |
| ATOM | 575 | O | GLY | 873 | 22.945 | 5.574 | 45.116 | 1.00 | 12.20 |
| ATOM | 576 | N | ALA | 874 | 23.274 | 7.645 | 45.860 | 1.00 | 12.84 |
| ATOM | 577 | H | ALA | 874 | 22.891 | 8.400 | 46.354 | 1.00 | 0.00 |
| ATOM | 578 | CA | ALA | 874 | 24.529 | 7.805 | 45.124 | 1.00 | 13.93 |
| ATOM | 579 | CB | ALA | 874 | 25.014 | 9.269 | 45.162 | 1.00 | 14.14 |
| ATOM | 580 | C | ALA | 874 | 25.668 | 6.906 | 45.693 | 1.00 | 14.11 |
| ATOM | 581 | O | ALA | 874 | 25.764 | 6.530 | 46.873 | 1.00 | 12.53 |
| ATOM | 582 | N | THR | 875 | 26.632 | 6.589 | 44.812 | 1.00 | 12.98 |
| ATOM | 583 | H | THR | 875 | 26.641 | 7.068 | 43.975 | 1.00 | 0.00 |
| ATOM | 584 | CA | THR | 875 | 27.759 | 5.726 | 45.140 | 1.00 | 11.86 |
| ATOM | 585 | CB | THR | 875 | 27.641 | 4.390 | 44.349 | 1.00 | 14.90 |
| ATOM | 586 | OG1 | THR | 875 | 27.934 | 4.704 | 42.976 | 1.00 | 14.75 |
| ATOM | 587 | HG1 | THR | 875 | 27.518 | 4.039 | 42.414 | 1.00 | 0.00 |
| ATOM | 588 | CG2 | THR | 875 | 26.234 | 3.718 | 44.434 | 1.00 | 13.55 |
| ATOM | 589 | C | THR | 875 | 29.002 | 6.441 | 44.713 | 1.00 | 12.64 |
| ATOM | 590 | O | THR | 875 | 28.887 | 7.513 | 44.103 | 1.00 | 9.74 |
| ATOM | 591 | N | HIS | 876 | 30.194 | 5.856 | 44.906 | 1.00 | 11.03 |
| ATOM | 592 | H | HIS | 876 | 30.223 | 4.988 | 45.370 | 1.00 | 0.00 |
| ATOM | 593 | CA | HIS | 876 | 31.444 | 6.519 | 44.505 | 1.00 | 14.10 |
| ATOM | 594 | C | HIS | 876 | 31.477 | 6.822 | 43.029 | 1.00 | 13.16 |
| ATOM | 595 | O | HIS | 876 | 32.194 | 7.716 | 42.624 | 1.00 | 13.12 |
| ATOM | 596 | CB | HIS | 876 | 32.705 | 5.676 | 44.898 | 1.00 | 21.58 |
| ATOM | 597 | CG | HIS | 876 | 33.979 | 6.352 | 44.372 | 1.00 | 35.85 |
| ATOM | 598 | ND1 | HIS | 876 | 34.370 | 7.682 | 44.526 | 1.00 | 37.81 |
| ATOM | 599 | HD1 | HIS | 876 | 33.912 | 8.323 | 45.105 | 1.00 | 0.00 |
| ATOM | 600 | CD2 | HIS | 876 | 35.062 | 5.744 | 43.665 | 1.00 | 39.00 |
| ATOM | 601 | NE2 | HIS | 876 | 36.077 | 6.624 | 43.386 | 1.00 | 38.34 |
| ATOM | 602 | CE1 | HIS | 876 | 35.628 | 7.831 | 43.934 | 1.00 | 40.44 |
| ATOM | 603 | N | SER | 877 | 30.754 | 6.129 | 42.175 | 1.00 | 13.37 |
| ATOM | 604 | H | SER | 877 | 30.196 | 5.390 | 42.499 | 1.00 | 0.00 |
| ATOM | 605 | CA | SER | 877 | 30.765 | 6.496 | 40.745 | 1.00 | 14.35 |
| ATOM | 606 | CB | SER | 877 | 30.200 | 5.338 | 39.797 | 1.00 | 18.27 |
| ATOM | 607 | OG | SER | 877 | 28.880 | 4.831 | 40.076 | 1.00 | 24.23 |
| ATOM | 608 | HG | SER | 877 | 28.552 | 4.369 | 39.295 | 1.00 | 0.00 |
| ATOM | 609 | C | SER | 877 | 29.984 | 7.808 | 40.535 | 1.00 | 13.40 |
| ATOM | 610 | O | SER | 877 | 30.405 | 8.594 | 39.702 | 1.00 | 12.57 |
| ATOM | 611 | N | GLU | 878 | 28.888 | 8.153 | 41.215 | 1.00 | 11.17 |
| ATOM | 612 | H | GLU | 878 | 28.495 | 7.492 | 41.824 | 1.00 | 0.00 |
| ATOM | 613 | CA | GLU | 878 | 28.285 | 9.486 | 40.990 | 1.00 | 10.51 |
| ATOM | 614 | CB | GLU | 878 | 26.917 | 9.609 | 41.670 | 1.00 | 10.65 |
| ATOM | 615 | CG | GLU | 878 | 25.748 | 8.988 | 40.919 | 1.00 | 10.67 |
| ATOM | 616 | CD | GLU | 878 | 25.796 | 7.474 | 41.022 | 1.00 | 15.84 |
| ATOM | 617 | OE1 | GLU | 878 | 26.239 | 6.881 | 42.006 | 1.00 | 15.83 |
| ATOM | 618 | OE2 | GLU | 878 | 25.371 | 6.835 | 40.085 | 1.00 | 18.23 |
| ATOM | 619 | C | GLU | 878 | 29.227 | 10.569 | 41.582 | 1.00 | 11.07 |
| ATOM | 620 | O | GLU | 878 | 29.356 | 11.691 | 41.125 | 1.00 | 10.59 |
| ATOM | 621 | N | HIS | 879 | 29.932 | 10.228 | 42.625 | 1.00 | 9.65 |
| ATOM | 622 | H | HIS | 879 | 29.715 | 9.370 | 43.049 | 1.00 | 0.00 |
| ATOM | 623 | CA | HIS | 879 | 30.901 | 11.106 | 43.263 | 1.00 | 10.11 |
| ATOM | 624 | C | HIS | 879 | 32.013 | 11.392 | 42.256 | 1.00 | 9.37 |
| ATOM | 625 | O | HIS | 879 | 32.428 | 12.546 | 42.076 | 1.00 | 8.98 |
| ATOM | 626 | CB | HIS | 879 | 31.344 | 10.334 | 44.514 | 1.00 | 11.24 |
| ATOM | 627 | CG | HIS | 879 | 32.196 | 11.185 | 45.308 | 1.00 | 14.65 |
| ATOM | 628 | ND1 | HIS | 879 | 33.533 | 10.974 | 45.448 | 1.00 | 17.57 |
| ATOM | 629 | HD1 | HIS | 879 | 34.092 | 10.374 | 44.912 | 1.00 | 0.00 |
| ATOM | 630 | CD2 | HIS | 879 | 31.827 | 12.319 | 46.032 | 1.00 | 14.39 |
| ATOM | 631 | NE2 | HIS | 879 | 32.966 | 12.779 | 46.619 | 1.00 | 15.92 |
| ATOM | 632 | CE1 | HIS | 879 | 34.012 | 11.964 | 46.258 | 1.00 | 16.03 |
| ATOM | 633 | N | ARG | 880 | 32.554 | 10.351 | 41.605 | 1.00 | 9.51 |
| ATOM | 634 | H | ARG | 880 | 32.308 | 9.442 | 41.850 | 1.00 | 0.00 |
| ATOM | 635 | CA | ARG | 880 | 33.592 | 10.566 | 40.530 | 1.00 | 12.66 |
| ATOM | 636 | CB | ARG | 880 | 34.042 | 9.208 | 39.925 | 1.00 | 14.20 |
| ATOM | 637 | CG | ARG | 880 | 35.066 | 8.405 | 40.717 | 1.00 | 17.16 |
| ATOM | 638 | CD | ARG | 880 | 35.655 | 7.236 | 39.888 | 1.00 | 19.04 |
| ATOM | 639 | NE | ARG | 880 | 34.589 | 6.255 | 39.714 | 1.00 | 26.91 |
| ATOM | 640 | HE | ARG | 880 | 33.843 | 6.440 | 39.107 | 1.00 | 0.00 |
| ATOM | 641 | CZ | ARG | 880 | 34.550 | 5.149 | 40.445 | 1.00 | 26.35 |
| ATOM | 642 | NH1 | ARG | 880 | 33.562 | 4.284 | 40.319 | 1.00 | 27.36 |
| ATOM | 643 | HH11 | ARG | 880 | 32.806 | 4.449 | 39.688 | 1.00 | 0.00 |
| ATOM | 644 | HH12 | ARG | 880 | 33.581 | 3.463 | 40.888 | 1.00 | 0.00 |
| ATOM | 645 | NH2 | ARG | 880 | 35.556 | 4.843 | 41.223 | 1.00 | 29.03 |
| ATOM | 646 | HH21 | ARG | 880 | 36.359 | 5.443 | 41.272 | 1.00 | 0.00 |
| ATOM | 647 | HH22 | ARG | 880 | 35.547 | 4.012 | 41.777 | 1.00 | 0.00 |
| ATOM | 648 | C | ARG | 880 | 32.994 | 11.454 | 39.374 | 1.00 | 11.85 |
| ATOM | 649 | O | ARG | 880 | 33.577 | 12.438 | 38.897 | 1.00 | 14.30 |
| ATOM | 650 | N | ALA | 881 | 31.743 | 11.183 | 38.915 | 1.00 | 11.66 |
| ATOM | 651 | H | ALA | 881 | 31.284 | 10.414 | 39.302 | 1.00 | 0.00 |
| ATOM | 652 | CA | ALA | 881 | 31.039 | 11.964 | 37.856 | 1.00 | 10.87 |
| ATOM | 653 | CB | ALA | 881 | 29.630 | 11.477 | 37.566 | 1.00 | 9.47 |
| ATOM | 654 | C | ALA | 881 | 30.845 | 13.425 | 38.246 | 1.00 | 11.23 |
| ATOM | 655 | O | ALA | 881 | 30.994 | 14.313 | 37.420 | 1.00 | 13.17 |
| ATOM | 656 | N | LEU | 882 | 30.509 | 13.728 | 39.508 | 1.00 | 9.60 |
| ATOM | 657 | H | LEU | 882 | 30.355 | 12.980 | 40.122 | 1.00 | 0.00 |
| ATOM | 658 | CA | LEU | 882 | 30.343 | 15.130 | 39.970 | 1.00 | 11.33 |
| ATOM | 659 | CB | LEU | 882 | 29.724 | 15.128 | 41.388 | 1.00 | 8.25 |
| ATOM | 660 | CG | LEU | 882 | 29.315 | 16.528 | 41.886 | 1.00 | 7.05 |
| ATOM | 661 | CD1 | LEU | 882 | 28.370 | 17.285 | 40.847 | 1.00 | 5.97 |
| ATOM | 662 | CD2 | LEU | 882 | 28.651 | 16.284 | 43.287 | 1.00 | 5.76 |
| ATOM | 663 | C | LEU | 882 | 31.700 | 15.907 | 39.947 | 1.00 | 11.93 |
| ATOM | 664 | O | LEU | 882 | 31.802 | 17.058 | 39.526 | 1.00 | 13.05 |
| ATOM | 665 | N | MET | 883 | 32.795 | 15.275 | 40.390 | 1.00 | 13.15 |
| ATOM | 666 | H | MET | 883 | 32.654 | 14.379 | 40.774 | 1.00 | 0.00 |
| ATOM | 667 | CA | MET | 883 | 34.147 | 15.835 | 40.382 | 1.00 | 12.66 |
| ATOM | 668 | CB | MET | 883 | 35.115 | 14.810 | 40.970 | 1.00 | 12.91 |
| ATOM | 669 | CG | MET | 883 | 36.551 | 15.377 | 41.020 | 1.00 | 17.19 |
| ATOM | 670 | SD | MET | 883 | 36.853 | 17.015 | 41.856 | 1.00 | 23.56 |
| ATOM | 671 | CE | MET | 883 | 36.205 | 16.532 | 43.404 | 1.00 | 19.99 |
| ATOM | 672 | C | MET | 883 | 34.523 | 16.115 | 38.936 | 1.00 | 12.45 |
| ATOM | 673 | O | MET | 883 | 34.994 | 17.195 | 38.587 | 1.00 | 11.36 |
| ATOM | 674 | N | SER | 884 | 34.310 | 15.169 | 38.028 | 1.00 | 11.85 |
| ATOM | 675 | H | SER | 884 | 33.948 | 14.291 | 38.272 | 1.00 | 0.00 |
| ATOM | 676 | CA | SER | 884 | 34.679 | 15.483 | 36.638 | 1.00 | 14.38 |
| ATOM | 677 | CB | SER | 884 | 34.705 | 14.107 | 35.779 | 1.00 | 12.00 |
| ATOM | 678 | OG | SER | 884 | 33.419 | 13.932 | 35.229 | 1.00 | 26.31 |
| ATOM | 679 | HG | SER | 884 | 33.359 | 13.051 | 34.848 | 1.00 | 0.00 |
| ATOM | 680 | C | SER | 884 | 33.758 | 16.618 | 36.084 | 1.00 | 13.88 |
| ATOM | 681 | O | SER | 884 | 34.194 | 17.406 | 35.231 | 1.00 | 14.26 |
| ATOM | 682 | N | GLU | 885 | 32.521 | 16.857 | 36.599 | 1.00 | 11.86 |
| ATOM | 683 | H | GLU | 885 | 32.155 | 16.184 | 37.209 | 1.00 | 0.00 |
| ATOM | 684 | CA | GLU | 885 | 31.637 | 17.977 | 36.155 | 1.00 | 10.69 |
| ATOM | 685 | CB | GLU | 885 | 30.214 | 17.780 | 36.703 | 1.00 | 11.58 |
| ATOM | 686 | CG | GLU | 885 | 29.337 | 19.045 | 36.659 | 1.00 | 11.18 |
| ATOM | 687 | CD | GLU | 885 | 27.891 | 18.756 | 37.023 | 1.00 | 12.41 |
| ATOM | 688 | OE1 | GLU | 885 | 27.548 | 17.627 | 37.362 | 1.00 | 15.34 |
| ATOM | 689 | OE2 | GLU | 885 | 27.090 | 19.676 | 36.925 | 1.00 | 10.58 |
| ATOM | 690 | C | GLU | 885 | 32.197 | 19.303 | 36.636 | 1.00 | 8.77 |
| ATOM | 691 | O | GLU | 885 | 32.131 | 20.355 | 35.997 | 1.00 | 9.14 |
| ATOM | 692 | N | LEU | 886 | 32.736 | 19.298 | 37.833 | 1.00 | 9.63 |
| ATOM | 693 | H | LEU | 886 | 32.592 | 18.513 | 38.394 | 1.00 | 0.00 |
| ATOM | 694 | CA | LEU | 886 | 33.423 | 20.464 | 38.414 | 1.00 | 9.82 |
| ATOM | 695 | CB | LEU | 886 | 33.886 | 20.070 | 39.874 | 1.00 | 8.32 |
| ATOM | 696 | CG | LEU | 886 | 34.915 | 20.999 | 40.584 | 1.00 | 6.06 |
| ATOM | 697 | CD1 | LEU | 886 | 34.313 | 22.417 | 40.568 | 1.00 | 6.33 |
| ATOM | 698 | CD2 | LEU | 886 | 35.319 | 20.464 | 41.990 | 1.00 | 4.44 |
| ATOM | 699 | C | LEU | 886 | 34.633 | 20.810 | 37.508 | 1.00 | 8.94 |
| ATOM | 700 | O | LEU | 886 | 34.904 | 21.957 | 37.149 | 1.00 | 9.81 |
| ATOM | 701 | N | LYS | 887 | 35.433 | 19.803 | 37.095 | 1.00 | 11.55 |
| ATOM | 702 | H | LYS | 887 | 35.268 | 18.909 | 37.447 | 1.00 | 0.00 |
| ATOM | 703 | CA | LYS | 887 | 36.631 | 20.036 | 36.233 | 1.00 | 10.60 |
| ATOM | 704 | CB | LYS | 887 | 37.360 | 18.715 | 36.003 | 1.00 | 8.26 |
| ATOM | 705 | CG | LYS | 887 | 38.208 | 18.552 | 37.279 | 1.00 | 9.43 |
| ATOM | 706 | CD | LYS | 887 | 38.563 | 17.086 | 37.312 | 1.00 | 10.42 |
| ATOM | 707 | CE | LYS | 887 | 39.439 | 16.661 | 38.477 | 1.00 | 13.75 |
| ATOM | 708 | NZ | LYS | 887 | 39.705 | 15.217 | 38.268 | 1.00 | 17.49 |
| ATOM | 709 | HZ1 | LYS | 887 | 40.065 | 15.086 | 37.301 | 1.00 | 0.00 |
| ATOM | 710 | HZ2 | LYS | 887 | 38.823 | 14.686 | 38.407 | 1.00 | 0.00 |
| ATOM | 711 | HZ3 | LYS | 887 | 40.425 | 14.886 | 38.943 | 1.00 | 0.00 |
| ATOM | 712 | C | LYS | 887 | 36.204 | 20.638 | 34.894 | 1.00 | 12.30 |
| ATOM | 713 | O | LYS | 887 | 36.713 | 21.645 | 34.392 | 1.00 | 13.21 |
| ATOM | 714 | N | ILE | 888 | 35.196 | 20.066 | 34.281 | 1.00 | 12.34 |
| ATOM | 715 | H | ILE | 888 | 34.817 | 19.262 | 34.647 | 1.00 | 0.00 |
| ATOM | 716 | CA | ILE | 888 | 34.749 | 20.621 | 32.986 | 1.00 | 13.85 |
| ATOM | 717 | CB | ILE | 888 | 33.654 | 19.687 | 32.213 | 1.00 | 14.25 |
| ATOM | 718 | CG2 | ILE | 888 | 32.217 | 19.810 | 32.662 | 1.00 | 11.68 |
| ATOM | 719 | CG1 | ILE | 888 | 33.688 | 20.187 | 30.707 | 1.00 | 15.34 |
| ATOM | 720 | CD1 | ILE | 888 | 35.039 | 19.917 | 29.937 | 1.00 | 14.57 |
| ATOM | 721 | C | ILE | 888 | 34.207 | 22.037 | 33.168 | 1.00 | 14.30 |
| ATOM | 722 | O | ILE | 888 | 34.554 | 22.909 | 32.370 | 1.00 | 15.86 |
| ATOM | 723 | N | LEU | 889 | 33.396 | 22.348 | 34.189 | 1.00 | 14.18 |
| ATOM | 724 | H | LEU | 889 | 33.078 | 21.643 | 34.786 | 1.00 | 0.00 |
| ATOM | 725 | CA | LEU | 889 | 32.905 | 23.722 | 34.391 | 1.00 | 11.22 |
| ATOM | 726 | CB | LEU | 889 | 32.021 | 23.806 | 35.674 | 1.00 | 14.31 |
| ATOM | 727 | CG | LEU | 889 | 30.644 | 23.171 | 35.448 | 1.00 | 13.24 |
| ATOM | 728 | CD1 | LEU | 889 | 29.906 | 23.057 | 36.772 | 1.00 | 11.60 |
| ATOM | 729 | CD2 | LEU | 889 | 29.884 | 24.034 | 34.467 | 1.00 | 15.26 |
| ATOM | 730 | C | LEU | 889 | 34.056 | 24.693 | 34.517 | 1.00 | 10.26 |
| ATOM | 731 | O | LEU | 889 | 33.872 | 25.847 | 34.156 | 1.00 | 8.28 |
| ATOM | 732 | N | ILE | 890 | 35.185 | 24.281 | 35.139 | 1.00 | 10.99 |
| ATOM | 733 | H | ILE | 890 | 35.195 | 23.378 | 35.535 | 1.00 | 0.00 |
| ATOM | 734 | CA | ILE | 890 | 36.363 | 25.130 | 35.282 | 1.00 | 9.57 |
| ATOM | 735 | CB | ILE | 890 | 37.400 | 24.419 | 36.183 | 1.00 | 9.63 |
| ATOM | 736 | CG2 | ILE | 890 | 38.693 | 25.280 | 36.075 | 1.00 | 8.92 |
| ATOM | 737 | CG1 | ILE | 890 | 36.945 | 24.288 | 37.710 | 1.00 | 7.16 |
| ATOM | 738 | CD1 | ILE | 890 | 37.959 | 23.632 | 38.679 | 1.00 | 3.52 |
| ATOM | 739 | C | ILE | 890 | 36.905 | 25.379 | 33.861 | 1.00 | 11.19 |
| ATOM | 740 | O | ILE | 890 | 37.154 | 26.529 | 33.492 | 1.00 | 10.84 |
| ATOM | 741 | N | HIS | 891 | 37.004 | 24.346 | 33.023 | 1.00 | 10.50 |
| ATOM | 742 | H | HIS | 891 | 36.844 | 23.449 | 33.396 | 1.00 | 0.00 |
| ATOM | 743 | CA | HIS | 891 | 37.504 | 24.516 | 31.653 | 1.00 | 11.48 |
| ATOM | 744 | C | HIS | 891 | 36.582 | 25.401 | 30.850 | 1.00 | 11.68 |
| ATOM | 745 | O | HIS | 891 | 36.992 | 26.304 | 30.148 | 1.00 | 11.27 |
| ATOM | 746 | CB | HIS | 891 | 37.629 | 23.121 | 31.091 | 1.00 | 12.09 |
| ATOM | 747 | CG | HIS | 891 | 37.646 | 23.232 | 29.637 | 1.00 | 16.19 |
| ATOM | 748 | ND1 | HIS | 891 | 38.729 | 23.607 | 28.914 | 1.00 | 15.09 |
| ATOM | 749 | CE1 | HIS | 891 | 38.329 | 23.649 | 27.590 | 1.00 | 13.72 |
| ATOM | 750 | CD2 | HIS | 891 | 36.553 | 23.020 | 28.765 | 1.00 | 15.26 |
| ATOM | 751 | NE2 | HIS | 891 | 37.006 | 23.279 | 27.525 | 1.00 | 15.01 |
| ATOM | 752 | HE2 | HIS | 891 | 36.498 | 23.200 | 26.718 | 1.00 | 0.00 |
| ATOM | 753 | N | ILE | 892 | 35.270 | 25.162 | 30.882 | 1.00 | 11.52 |
| ATOM | 754 | H | ILE | 892 | 34.955 | 24.441 | 31.442 | 1.00 | 0.00 |
| ATOM | 755 | CA | ILE | 892 | 34.327 | 25.990 | 30.156 | 1.00 | 8.99 |
| ATOM | 756 | CB | ILE | 892 | 32.863 | 25.478 | 30.495 | 1.00 | 9.17 |
| ATOM | 757 | CG2 | ILE | 892 | 31.863 | 26.603 | 30.148 | 1.00 | 7.47 |
| ATOM | 758 | CG1 | ILE | 892 | 32.536 | 24.105 | 29.779 | 1.00 | 6.76 |
| ATOM | 759 | CD1 | ILE | 892 | 31.391 | 23.281 | 30.526 | 1.00 | 6.58 |
| ATOM | 760 | C | ILE | 892 | 34.526 | 27.465 | 30.570 | 1.00 | 10.35 |
| ATOM | 761 | O | ILE | 892 | 34.673 | 28.345 | 29.758 | 1.00 | 8.76 |
| ATOM | 762 | N | GLY | 893 | 34.570 | 27.829 | 31.851 | 1.00 | 11.62 |
| ATOM | 763 | H | GLY | 893 | 34.598 | 27.118 | 32.530 | 1.00 | 0.00 |
| ATOM | 764 | CA | GLY | 893 | 34.693 | 29.237 | 32.254 | 1.00 | 11.93 |
| ATOM | 765 | C | GLY | 893 | 33.344 | 30.057 | 32.270 | 1.00 | 14.13 |
| ATOM | 766 | O | GLY | 893 | 32.206 | 29.576 | 32.137 | 1.00 | 15.49 |
| ATOM | 767 | N | HIS | 894 | 33.435 | 31.369 | 32.377 | 1.00 | 11.26 |
| ATOM | 768 | H | HIS | 894 | 34.278 | 31.761 | 32.125 | 1.00 | 0.00 |
| ATOM | 769 | CA | HIS | 894 | 32.236 | 32.184 | 32.469 | 1.00 | 10.93 |
| ATOM | 770 | C | HIS | 894 | 31.743 | 32.926 | 31.246 | 1.00 | 10.39 |
| ATOM | 771 | O | HIS | 894 | 32.500 | 33.459 | 30.429 | 1.00 | 12.57 |
| ATOM | 772 | CB | HIS | 894 | 32.450 | 33.194 | 33.681 | 1.00 | 13.57 |
| ATOM | 773 | CG | HIS | 894 | 33.691 | 34.071 | 33.601 | 1.00 | 14.89 |
| ATOM | 774 | ND1 | HIS | 894 | 33.744 | 35.299 | 33.019 | 1.00 | 16.04 |
| ATOM | 775 | CE1 | HIS | 894 | 35.058 | 35.749 | 33.011 | 1.00 | 14.74 |
| ATOM | 776 | CD2 | HIS | 894 | 35.031 | 33.792 | 33.960 | 1.00 | 15.50 |
| ATOM | 777 | NE2 | HIS | 894 | 35.860 | 34.823 | 33.599 | 1.00 | 15.53 |
| ATOM | 778 | HE2 | HIS | 894 | 36.797 | 34.977 | 33.854 | 1.00 | 0.00 |
| ATOM | 779 | N | HIS | 895 | 30.421 | 33.063 | 31.213 | 1.00 | 9.72 |
| ATOM | 780 | H | HIS | 895 | 29.897 | 32.569 | 31.885 | 1.00 | 0.00 |
| ATOM | 781 | CA | HIS | 895 | 29.737 | 33.790 | 30.154 | 1.00 | 8.97 |
| ATOM | 782 | C | HIS | 895 | 28.436 | 34.180 | 30.750 | 1.00 | 8.86 |
| ATOM | 783 | O | HIS | 895 | 27.906 | 33.479 | 31.593 | 1.00 | 11.39 |
| ATOM | 784 | CB | HIS | 895 | 29.506 | 32.881 | 28.912 | 1.00 | 9.29 |
| ATOM | 785 | CG | HIS | 895 | 28.820 | 33.609 | 27.852 | 1.00 | 12.10 |
| ATOM | 786 | ND1 | HIS | 895 | 27.467 | 33.723 | 27.729 | 1.00 | 13.34 |
| ATOM | 787 | CE1 | HIS | 895 | 27.204 | 34.473 | 26.628 | 1.00 | 12.38 |
| ATOM | 788 | CD2 | HIS | 895 | 29.407 | 34.309 | 26.799 | 1.00 | 10.58 |
| ATOM | 789 | NE2 | HIS | 895 | 28.389 | 34.815 | 26.057 | 1.00 | 12.40 |
| ATOM | 790 | HE2 | HIS | 895 | 28.498 | 35.147 | 25.147 | 1.00 | 0.00 |
| ATOM | 791 | N | LEU | 896 | 27.855 | 35.282 | 30.348 | 1.00 | 10.20 |
| ATOM | 792 | H | LEU | 896 | 28.341 | 35.868 | 29.735 | 1.00 | 0.00 |
| ATOM | 793 | CA | LEU | 896 | 26.545 | 35.720 | 30.885 | 1.00 | 8.66 |
| ATOM | 794 | CB | LEU | 896 | 26.057 | 37.028 | 30.153 | 1.00 | 7.86 |
| ATOM | 795 | CG | LEU | 896 | 24.713 | 37.525 | 30.691 | 1.00 | 12.17 |
| ATOM | 796 | CD1 | LEU | 896 | 25.045 | 38.093 | 32.085 | 1.00 | 6.73 |
| ATOM | 797 | CD2 | LEU | 896 | 23.945 | 38.431 | 29.677 | 1.00 | 10.66 |
| ATOM | 798 | C | LEU | 896 | 25.453 | 34.661 | 30.720 | 1.00 | 9.55 |
| ATOM | 799 | O | LEU | 896 | 24.585 | 34.436 | 31.572 | 1.00 | 8.49 |
| ATOM | 800 | N | ASN | 897 | 25.453 | 34.032 | 29.540 | 1.00 | 10.93 |
| ATOM | 801 | H | ASN | 897 | 26.134 | 34.289 | 28.903 | 1.00 | 0.00 |
| ATOM | 802 | CA | ASN | 897 | 24.389 | 33.062 | 29.228 | 1.00 | 11.08 |
| ATOM | 803 | CB | ASN | 897 | 24.015 | 33.235 | 27.705 | 1.00 | 8.76 |
| ATOM | 804 | CG | ASN | 897 | 23.536 | 34.643 | 27.556 | 1.00 | 10.41 |
| ATOM | 805 | OD1 | ASN | 897 | 24.086 | 35.417 | 26.813 | 1.00 | 7.25 |
| ATOM | 806 | ND2 | ASN | 897 | 22.558 | 35.120 | 28.291 | 1.00 | 13.03 |
| ATOM | 807 | HD21 | ASN | 897 | 22.111 | 34.562 | 28.963 | 1.00 | 0.00 |
| ATOM | 808 | HD22 | ASN | 897 | 22.268 | 36.039 | 28.165 | 1.00 | 0.00 |
| ATOM | 809 | C | ASN | 897 | 24.549 | 31.599 | 29.602 | 1.00 | 11.48 |
| ATOM | 810 | O | ASN | 897 | 23.807 | 30.746 | 29.113 | 1.00 | 8.13 |
| ATOM | 811 | N | VAL | 898 | 25.535 | 31.298 | 30.466 | 1.00 | 13.16 |
| ATOM | 812 | H | VAL | 898 | 26.094 | 32.021 | 30.826 | 1.00 | 0.00 |
| ATOM | 813 | CA | VAL | 898 | 25.653 | 29.913 | 30.992 | 1.00 | 12.94 |
| ATOM | 814 | CB | VAL | 898 | 26.974 | 29.194 | 30.488 | 1.00 | 11.16 |
| ATOM | 815 | CG1 | VAL | 898 | 26.956 | 28.966 | 28.948 | 1.00 | 5.53 |
| ATOM | 816 | CG2 | VAL | 898 | 28.152 | 29.993 | 31.051 | 1.00 | 10.80 |
| ATOM | 817 | C | VAL | 898 | 25.662 | 30.007 | 32.548 | 1.00 | 14.86 |
| ATOM | 818 | O | VAL | 898 | 26.012 | 31.052 | 33.113 | 1.00 | 14.62 |
| ATOM | 819 | N | VAL | 899 | 25.149 | 29.028 | 33.312 | 1.00 | 15.74 |
| ATOM | 820 | H | VAL | 899 | 24.756 | 28.234 | 32.895 | 1.00 | 0.00 |
| ATOM | 821 | CA | VAL | 899 | 25.291 | 29.152 | 34.790 | 1.00 | 15.93 |
| ATOM | 822 | CB | VAL | 899 | 24.390 | 28.010 | 35.347 | 1.00 | 16.17 |
| ATOM | 823 | CG1 | VAL | 899 | 25.241 | 26.782 | 35.611 | 1.00 | 15.81 |
| ATOM | 824 | CG2 | VAL | 899 | 23.592 | 28.517 | 36.516 | 1.00 | 19.65 |
| ATOM | 825 | C | VAL | 899 | 26.845 | 29.067 | 35.089 | 1.00 | 15.49 |
| ATOM | 826 | O | VAL | 899 | 27.587 | 28.238 | 34.551 | 1.00 | 16.01 |
| ATOM | 827 | N | ASN | 900 | 27.415 | 30.001 | 35.844 | 1.00 | 12.82 |
| ATOM | 828 | H | ASN | 900 | 26.826 | 30.654 | 36.232 | 1.00 | 0.00 |
| ATOM | 829 | CA | ASN | 900 | 28.862 | 30.035 | 36.141 | 1.00 | 13.41 |
| ATOM | 830 | CB | ASN | 900 | 29.494 | 31.510 | 36.088 | 1.00 | 7.97 |
| ATOM | 831 | CG | ASN | 900 | 29.265 | 32.076 | 34.703 | 1.00 | 7.89 |
| ATOM | 832 | OD1 | ASN | 900 | 29.586 | 31.476 | 33.691 | 1.00 | 10.97 |
| ATOM | 833 | ND2 | ASN | 900 | 28.672 | 33.229 | 34.520 | 1.00 | 8.33 |
| ATOM | 834 | HD21 | ASN | 900 | 28.350 | 33.781 | 35.254 | 1.00 | 0.00 |
| ATOM | 835 | HD22 | ASN | 900 | 28.545 | 33.527 | 33.592 | 1.00 | 0.00 |
| ATOM | 836 | C | ASN | 900 | 29.226 | 29.464 | 37.482 | 1.00 | 12.23 |
| ATOM | 837 | O | ASN | 900 | 28.476 | 29.635 | 38.455 | 1.00 | 13.37 |
| ATOM | 838 | N | LEU | 901 | 30.333 | 28.726 | 37.441 | 1.00 | 12.65 |
| ATOM | 839 | H | LEU | 901 | 30.784 | 28.652 | 36.567 | 1.00 | 0.00 |
| ATOM | 840 | CA | LEU | 901 | 30.960 | 28.087 | 38.618 | 1.00 | 12.86 |
| ATOM | 841 | CB | LEU | 901 | 32.141 | 27.127 | 38.200 | 1.00 | 11.90 |
| ATOM | 842 | CG | LEU | 901 | 32.929 | 26.493 | 39.383 | 1.00 | 9.99 |
| ATOM | 843 | CD1 | LEU | 901 | 32.027 | 25.578 | 40.165 | 1.00 | 7.79 |
| ATOM | 844 | CD2 | LEU | 901 | 34.102 | 25.715 | 38.870 | 1.00 | 7.33 |
| ATOM | 845 | C | LEU | 901 | 31.533 | 29.202 | 39.508 | 1.00 | 13.48 |
| ATOM | 846 | O | LEU | 901 | 32.288 | 30.052 | 39.014 | 1.00 | 13.90 |
| ATOM | 847 | N | LEU | 902 | 31.094 | 29.257 | 40.776 | 1.00 | 13.00 |
| ATOM | 848 | H | LEU | 902 | 30.349 | 28.665 | 40.977 | 1.00 | 0.00 |
| ATOM | 849 | CA | LEU | 902 | 31.537 | 30.241 | 41.845 | 1.00 | 14.02 |
| ATOM | 850 | CB | LEU | 902 | 30.265 | 30.742 | 42.646 | 1.00 | 9.41 |
| ATOM | 851 | CG | LEU | 902 | 29.216 | 31.414 | 41.758 | 1.00 | 7.80 |
| ATOM | 852 | CD1 | LEU | 902 | 28.094 | 31.795 | 42.652 | 1.00 | 5.80 |
| ATOM | 853 | CD2 | LEU | 902 | 29.754 | 32.656 | 41.053 | 1.00 | 8.43 |
| ATOM | 854 | C | LEU | 902 | 32.585 | 29.644 | 42.840 | 1.00 | 14.35 |
| ATOM | 855 | O | LEU | 902 | 33.521 | 30.267 | 43.346 | 1.00 | 14.73 |
| ATOM | 856 | N | GLY | 903 | 32.485 | 28.361 | 43.179 | 1.00 | 15.98 |
| ATOM | 857 | H | GLY | 903 | 31.776 | 27.830 | 42.767 | 1.00 | 0.00 |
| ATOM | 858 | CA | GLY | 903 | 33.429 | 27.736 | 44.138 | 1.00 | 12.37 |
| ATOM | 859 | C | GLY | 903 | 33.084 | 26.284 | 44.293 | 1.00 | 12.39 |
| ATOM | 860 | O | GLY | 903 | 32.133 | 25.784 | 43.647 | 1.00 | 12.78 |
| ATOM | 861 | N | ALA | 904 | 33.867 | 25.568 | 45.093 | 1.00 | 11.63 |
| ATOM | 862 | H | ALA | 904 | 34.645 | 26.026 | 45.481 | 1.00 | 0.00 |
| ATOM | 863 | CA | ALA | 904 | 33.591 | 24.118 | 45.360 | 1.00 | 13.37 |
| ATOM | 864 | CB | ALA | 904 | 34.239 | 23.168 | 44.353 | 1.00 | 8.48 |
| ATOM | 865 | C | ALA | 904 | 34.086 | 23.624 | 46.721 | 1.00 | 13.03 |
| ATOM | 866 | O | ALA | 904 | 35.059 | 24.158 | 47.264 | 1.00 | 13.67 |
| ATOM | 867 | N | CYS | 905 | 33.379 | 22.647 | 47.329 | 1.00 | 12.13 |
| ATOM | 868 | H | CYS | 905 | 32.564 | 22.326 | 46.881 | 1.00 | 0.00 |
| ATOM | 869 | CA | CYS | 905 | 33.783 | 22.078 | 48.622 | 1.00 | 10.51 |
| ATOM | 870 | CB | CYS | 905 | 32.677 | 22.109 | 49.612 | 1.00 | 10.95 |
| ATOM | 871 | SG | CYS | 905 | 31.922 | 23.744 | 49.804 | 1.00 | 12.03 |
| ATOM | 872 | C | CYS | 905 | 34.103 | 20.657 | 48.321 | 1.00 | 10.18 |
| ATOM | 873 | O | CYS | 905 | 33.273 | 19.837 | 47.999 | 1.00 | 9.79 |
| ATOM | 874 | N | THR | 906 | 35.366 | 20.380 | 48.193 | 1.00 | 10.82 |
| ATOM | 875 | H | THR | 906 | 35.970 | 21.144 | 48.149 | 1.00 | 0.00 |
| ATOM | 876 | CA | THR | 906 | 35.887 | 19.029 | 47.846 | 1.00 | 12.84 |
| ATOM | 877 | CB | THR | 906 | 36.734 | 19.146 | 46.527 | 1.00 | 10.44 |
| ATOM | 878 | OG1 | THR | 906 | 38.007 | 19.666 | 46.918 | 1.00 | 10.05 |
| ATOM | 879 | HG1 | THR | 906 | 38.620 | 19.640 | 46.174 | 1.00 | 0.00 |
| ATOM | 880 | CG2 | THR | 906 | 36.134 | 20.085 | 45.478 | 1.00 | 6.51 |
| ATOM | 881 | C | THR | 906 | 36.757 | 18.374 | 48.958 | 1.00 | 14.70 |
| ATOM | 882 | O | THR | 906 | 37.254 | 17.260 | 48.850 | 1.00 | 14.82 |
| ATOM | 883 | N | LYS | 907 | 37.039 | 19.108 | 50.047 | 1.00 | 16.65 |
| ATOM | 884 | H | LYS | 907 | 36.607 | 19.976 | 50.134 | 1.00 | 0.00 |
| ATOM | 885 | CA | LYS | 907 | 37.813 | 18.549 | 51.165 | 1.00 | 17.00 |
| ATOM | 886 | CB | LYS | 907 | 38.052 | 19.698 | 52.229 | 1.00 | 16.13 |
| ATOM | 887 | C | LYS | 907 | 37.143 | 17.308 | 51.853 | 1.00 | 16.52 |
| ATOM | 888 | O | LYS | 907 | 35.915 | 17.084 | 51.967 | 1.00 | 13.15 |
| ATOM | 889 | N | PRO | 908 | 37.984 | 16.402 | 52.348 | 1.00 | 18.08 |
| ATOM | 890 | CD | PRO | 908 | 39.364 | 16.169 | 51.939 | 1.00 | 17.90 |
| ATOM | 891 | CA | PRO | 908 | 37.489 | 15.221 | 53.035 | 1.00 | 18.60 |
| ATOM | 892 | CB | PRO | 908 | 38.705 | 14.378 | 53.482 | 1.00 | 17.41 |
| ATOM | 893 | CG | PRO | 908 | 39.840 | 15.317 | 53.129 | 1.00 | 19.13 |
| ATOM | 894 | C | PRO | 908 | 36.527 | 15.359 | 54.180 | 1.00 | 20.57 |
| ATOM | 895 | O | PRO | 908 | 35.747 | 14.410 | 54.300 | 1.00 | 23.93 |
| ATOM | 896 | N | GLY | 909 | 36.370 | 16.338 | 55.073 | 1.00 | 19.86 |
| ATOM | 897 | H | GLY | 909 | 36.862 | 17.176 | 55.066 | 1.00 | 0.00 |
| ATOM | 898 | CA | GLY | 909 | 35.214 | 16.024 | 56.068 | 1.00 | 19.86 |
| ATOM | 899 | C | GLY | 909 | 33.699 | 15.891 | 55.587 | 1.00 | 17.24 |
| ATOM | 900 | O | GLY | 909 | 32.819 | 15.503 | 56.357 | 1.00 | 15.14 |
| ATOM | 901 | N | GLY | 910 | 33.338 | 16.134 | 54.303 | 1.00 | 14.35 |
| ATOM | 902 | H | GLY | 910 | 34.038 | 16.191 | 53.621 | 1.00 | 0.00 |
| ATOM | 903 | CA | GLY | 910 | 31.942 | 16.137 | 53.911 | 1.00 | 10.43 |
| ATOM | 904 | C | GLY | 910 | 31.635 | 15.959 | 52.455 | 1.00 | 10.53 |
| ATOM | 905 | O | GLY | 910 | 32.475 | 15.447 | 51.702 | 1.00 | 7.91 |
| ATOM | 906 | N | PRO | 911 | 30.402 | 16.285 | 52.035 | 1.00 | 10.45 |
| ATOM | 907 | CD | PRO | 911 | 29.331 | 16.945 | 52.791 | 1.00 | 10.48 |
| ATOM | 908 | CA | PRO | 911 | 30.006 | 15.994 | 50.633 | 1.00 | 12.76 |
| ATOM | 909 | CB | PRO | 911 | 28.482 | 16.113 | 50.666 | 1.00 | 9.83 |
| ATOM | 910 | CG | PRO | 911 | 28.270 | 17.148 | 51.723 | 1.00 | 12.02 |
| ATOM | 911 | C | PRO | 911 | 30.680 | 16.876 | 49.511 | 1.00 | 14.50 |
| ATOM | 912 | O | PRO | 911 | 31.057 | 18.049 | 49.740 | 1.00 | 12.66 |
| ATOM | 913 | N | LEU | 912 | 30.913 | 16.317 | 48.296 | 1.00 | 12.62 |
| ATOM | 914 | H | LEU | 912 | 30.706 | 15.371 | 48.134 | 1.00 | 0.00 |
| ATOM | 915 | CA | LEU | 912 | 31.483 | 17.159 | 47.229 | 1.00 | 11.37 |
| ATOM | 916 | CB | LEU | 912 | 31.958 | 16.233 | 46.051 | 1.00 | 10.41 |
| ATOM | 917 | CG | LEU | 912 | 32.280 | 16.899 | 44.667 | 1.00 | 11.03 |
| ATOM | 918 | CD1 | LEU | 912 | 33.402 | 17.913 | 44.741 | 1.00 | 5.88 |
| ATOM | 919 | CD2 | LEU | 912 | 32.544 | 15.749 | 43.686 | 1.00 | 8.09 |
| ATOM | 920 | C | LEU | 912 | 30.336 | 18.124 | 46.820 | 1.00 | 12.24 |
| ATOM | 921 | O | LEU | 912 | 29.216 | 17.710 | 46.533 | 1.00 | 13.32 |
| ATOM | 922 | N | MET | 913 | 30.562 | 19.440 | 46.839 | 1.00 | 11.24 |
| ATOM | 923 | H | MET | 913 | 31.449 | 19.743 | 47.131 | 1.00 | 0.00 |
| ATOM | 924 | CA | MET | 913 | 29.520 | 20.432 | 46.485 | 1.00 | 10.20 |
| ATOM | 925 | CB | MET | 913 | 29.240 | 21.277 | 47.707 | 1.00 | 11.60 |
| ATOM | 926 | CG | MET | 913 | 28.683 | 20.393 | 48.823 | 1.00 | 14.55 |
| ATOM | 927 | SD | MET | 913 | 28.631 | 21.364 | 50.315 | 1.00 | 21.35 |
| ATOM | 928 | CE | MET | 913 | 27.082 | 20.768 | 50.897 | 1.00 | 24.13 |
| ATOM | 929 | C | MET | 913 | 30.064 | 21.315 | 45.348 | 1.00 | 12.17 |
| ATOM | 930 | O | MET | 913 | 31.228 | 21.769 | 45.443 | 1.00 | 11.45 |
| ATOM | 931 | N | VAL | 914 | 29.228 | 21.555 | 44.296 | 1.00 | 9.66 |
| ATOM | 932 | H | VAL | 914 | 28.344 | 21.136 | 44.307 | 1.00 | 0.00 |
| ATOM | 933 | CA | VAL | 914 | 29.633 | 22.345 | 43.106 | 1.00 | 9.42 |
| ATOM | 934 | CB | VAL | 914 | 29.564 | 21.442 | 41.859 | 1.00 | 11.05 |
| ATOM | 935 | CG1 | VAL | 914 | 30.171 | 22.256 | 40.678 | 1.00 | 8.79 |
| ATOM | 936 | CG2 | VAL | 914 | 30.374 | 20.105 | 42.076 | 1.00 | 5.33 |
| ATOM | 937 | C | VAL | 914 | 28.673 | 23.479 | 43.084 | 1.00 | 10.61 |
| ATOM | 938 | O | VAL | 914 | 27.479 | 23.261 | 42.974 | 1.00 | 11.08 |
| ATOM | 939 | N | ILE | 915 | 29.244 | 24.656 | 43.417 | 1.00 | 11.87 |
| ATOM | 940 | H | ILE | 915 | 30.222 | 24.664 | 43.492 | 1.00 | 0.00 |
| ATOM | 941 | CA | ILE | 915 | 28.553 | 25.937 | 43.602 | 1.00 | 9.89 |
| ATOM | 942 | CB | ILE | 915 | 29.186 | 26.685 | 44.826 | 1.00 | 8.40 |
| ATOM | 943 | CG2 | ILE | 915 | 28.258 | 27.859 | 45.158 | 1.00 | 7.17 |
| ATOM | 944 | CG1 | ILE | 915 | 29.347 | 25.792 | 46.069 | 1.00 | 7.35 |
| ATOM | 945 | CD1 | ILE | 915 | 30.446 | 26.351 | 47.002 | 1.00 | 8.40 |
| ATOM | 946 | C | ILE | 915 | 28.556 | 26.844 | 42.413 | 1.00 | 11.10 |
| ATOM | 947 | O | ILE | 915 | 29.607 | 27.293 | 41.969 | 1.00 | 11.91 |
| ATOM | 948 | N | VAL | 916 | 27.353 | 27.137 | 41.913 | 1.00 | 11.04 |
| ATOM | 949 | H | VAL | 916 | 26.566 | 26.747 | 42.359 | 1.00 | 0.00 |
| ATOM | 950 | CA | VAL | 916 | 27.125 | 28.027 | 40.771 | 1.00 | 9.88 |
| ATOM | 951 | CB | VAL | 916 | 26.552 | 27.262 | 39.570 | 1.00 | 5.91 |
| ATOM | 952 | CG1 | VAL | 916 | 27.525 | 26.163 | 39.162 | 1.00 | 5.76 |
| ATOM | 953 | CG2 | VAL | 916 | 25.105 | 26.727 | 39.938 | 1.00 | 4.31 |
| ATOM | 954 | C | VAL | 916 | 26.116 | 29.139 | 41.139 | 1.00 | 13.36 |
| ATOM | 955 | O | VAL | 916 | 25.513 | 29.196 | 42.235 | 1.00 | 14.83 |
| ATOM | 956 | N | GLU | 917 | 25.917 | 30.068 | 40.186 | 1.00 | 14.30 |
| ATOM | 957 | H | GLU | 917 | 26.480 | 30.002 | 39.382 | 1.00 | 0.00 |
| ATOM | 958 | CA | GLU | 917 | 24.952 | 31.203 | 40.316 | 1.00 | 12.81 |
| ATOM | 959 | CB | GLU | 917 | 24.806 | 32.066 | 39.043 | 1.00 | 13.58 |
| ATOM | 960 | CG | GLU | 917 | 26.223 | 32.665 | 38.785 | 1.00 | 14.81 |
| ATOM | 961 | CD | GLU | 917 | 26.356 | 33.150 | 37.333 | 1.00 | 17.64 |
| ATOM | 962 | OE1 | GLU | 917 | 26.897 | 34.234 | 37.115 | 1.00 | 16.91 |
| ATOM | 963 | OE2 | GLU | 917 | 25.942 | 32.420 | 36.435 | 1.00 | 16.91 |
| ATOM | 964 | C | GLU | 917 | 23.548 | 30.804 | 40.571 | 1.00 | 11.24 |
| ATOM | 965 | O | GLU | 917 | 23.091 | 29.853 | 39.942 | 1.00 | 11.29 |
| ATOM | 966 | N | PHE | 918 | 22.865 | 31.498 | 41.487 | 1.00 | 10.97 |
| ATOM | 967 | H | PHE | 918 | 23.355 | 32.140 | 42.035 | 1.00 | 0.00 |
| ATOM | 968 | CA | PHE | 918 | 21.439 | 31.279 | 41.728 | 1.00 | 9.11 |
| ATOM | 969 | CB | PHE | 918 | 21.092 | 31.690 | 43.140 | 1.00 | 9.90 |
| ATOM | 970 | CG | PHE | 918 | 19.659 | 31.603 | 43.355 | 1.00 | 7.87 |
| ATOM | 971 | CD1 | PHE | 918 | 19.035 | 30.360 | 43.389 | 1.00 | 9.84 |
| ATOM | 972 | CD2 | PHE | 918 | 18.938 | 32.780 | 43.563 | 1.00 | 7.10 |
| ATOM | 973 | CE1 | PHE | 918 | 17.663 | 30.285 | 43.649 | 1.00 | 10.17 |
| ATOM | 974 | CE2 | PHE | 918 | 17.567 | 32.697 | 43.824 | 1.00 | 9.85 |
| ATOM | 975 | CZ | PHE | 918 | 16.941 | 31.450 | 43.867 | 1.00 | 11.22 |
| ATOM | 976 | C | PHE | 918 | 20.639 | 32.176 | 40.720 | 1.00 | 11.97 |
| ATOM | 977 | O | PHE | 918 | 20.976 | 33.369 | 40.532 | 1.00 | 13.58 |
| ATOM | 978 | N | CYS | 919 | 19.668 | 31.567 | 39.992 | 1.00 | 12.60 |
| ATOM | 979 | H | CYS | 919 | 19.611 | 30.610 | 40.167 | 1.00 | 0.00 |
| ATOM | 980 | CA | CYS | 919 | 18.706 | 32.130 | 38.990 | 1.00 | 9.86 |
| ATOM | 981 | CB | CYS | 919 | 18.721 | 31.278 | 37.733 | 1.00 | 9.10 |
| ATOM | 982 | SG | CYS | 919 | 20.412 | 31.388 | 37.041 | 1.00 | 12.96 |
| ATOM | 983 | C | CYS | 919 | 17.281 | 32.188 | 39.559 | 1.00 | 10.14 |
| ATOM | 984 | O | CYS | 919 | 16.535 | 31.218 | 39.604 | 1.00 | 9.92 |
| ATOM | 985 | N | LYS | 920 | 16.982 | 33.351 | 40.093 | 1.00 | 10.17 |
| ATOM | 986 | H | LYS | 920 | 17.708 | 34.007 | 40.053 | 1.00 | 0.00 |
| ATOM | 987 | CA | LYS | 920 | 15.734 | 33.734 | 40.756 | 1.00 | 14.30 |
| ATOM | 988 | CB | LYS | 920 | 15.747 | 35.277 | 40.677 | 1.00 | 20.03 |
| ATOM | 989 | CG | LYS | 920 | 14.643 | 36.205 | 41.139 | 1.00 | 25.67 |
| ATOM | 990 | CD | LYS | 920 | 15.369 | 37.070 | 42.150 | 1.00 | 37.75 |
| ATOM | 991 | CE | LYS | 920 | 15.811 | 38.498 | 41.665 | 1.00 | 43.27 |
| ATOM | 992 | NZ | LYS | 920 | 16.947 | 39.053 | 42.470 | 1.00 | 50.03 |
| ATOM | 993 | HZ1 | LYS | 920 | 17.581 | 38.281 | 42.769 | 1.00 | 0.00 |
| ATOM | 994 | HZ2 | LYS | 920 | 16.638 | 39.569 | 43.323 | 1.00 | 0.00 |
| ATOM | 995 | HZ3 | LYS | 920 | 17.477 | 39.706 | 41.859 | 1.00 | 0.00 |
| ATOM | 996 | C | LYS | 920 | 14.414 | 33.137 | 40.287 | 1.00 | 12.85 |
| ATOM | 997 | O | LYS | 920 | 13.617 | 32.757 | 41.113 | 1.00 | 12.85 |
| ATOM | 998 | N | PHE | 921 | 14.121 | 33.063 | 38.989 | 1.00 | 13.43 |
| ATOM | 999 | H | PHE | 921 | 14.803 | 33.376 | 38.364 | 1.00 | 0.00 |
| ATOM | 1000 | CA | PHE | 921 | 12.862 | 32.550 | 38.416 | 1.00 | 12.30 |
| ATOM | 1001 | CB | PHE | 921 | 12.613 | 33.385 | 37.181 | 1.00 | 14.41 |
| ATOM | 1002 | CG | PHE | 921 | 12.312 | 34.771 | 37.563 | 1.00 | 17.64 |
| ATOM | 1003 | CD1 | PHE | 921 | 11.024 | 35.111 | 38.043 | 1.00 | 21.95 |
| ATOM | 1004 | CD2 | PHE | 921 | 13.299 | 35.753 | 37.472 | 1.00 | 16.78 |
| ATOM | 1005 | CE1 | PHE | 921 | 10.739 | 36.435 | 38.434 | 1.00 | 23.57 |
| ATOM | 1006 | CE2 | PHE | 921 | 13.041 | 37.079 | 37.853 | 1.00 | 19.36 |
| ATOM | 1007 | CZ | PHE | 921 | 11.774 | 37.407 | 38.331 | 1.00 | 23.09 |
| ATOM | 1008 | C | PHE | 921 | 12.810 | 31.086 | 38.082 | 1.00 | 13.65 |
| ATOM | 1009 | O | PHE | 921 | 11.831 | 30.538 | 37.587 | 1.00 | 15.74 |
| ATOM | 1010 | N | GLY | 922 | 13.925 | 30.401 | 38.271 | 1.00 | 14.61 |
| ATOM | 1011 | H | GLY | 922 | 14.695 | 30.867 | 38.648 | 1.00 | 0.00 |
| ATOM | 1012 | CA | GLY | 922 | 13.955 | 28.969 | 37.982 | 1.00 | 15.59 |
| ATOM | 1013 | C | GLY | 922 | 13.952 | 28.665 | 36.502 | 1.00 | 15.93 |
| ATOM | 1014 | O | GLY | 922 | 14.221 | 29.510 | 35.636 | 1.00 | 15.04 |
| ATOM | 1015 | N | ASN | 923 | 13.638 | 27.419 | 36.198 | 1.00 | 16.36 |
| ATOM | 1016 | H | ASN | 923 | 13.551 | 26.762 | 36.919 | 1.00 | 0.00 |
| ATOM | 1017 | CA | ASN | 923 | 13.664 | 27.012 | 34.776 | 1.00 | 13.91 |
| ATOM | 1018 | CB | ASN | 923 | 13.604 | 25.464 | 34.745 | 1.00 | 14.04 |
| ATOM | 1019 | CG | ASN | 923 | 12.231 | 24.879 | 34.662 | 1.00 | 12.96 |
| ATOM | 1020 | OD1 | ASN | 923 | 11.672 | 24.736 | 33.590 | 1.00 | 15.83 |
| ATOM | 1021 | ND2 | ASN | 923 | 11.619 | 24.535 | 35.760 | 1.00 | 15.75 |
| ATOM | 1022 | HD21 | ASN | 923 | 12.053 | 24.668 | 36.626 | 1.00 | 0.00 |
| ATOM | 1023 | HD22 | ASN | 923 | 10.727 | 24.154 | 35.665 | 1.00 | 0.00 |
| ATOM | 1024 | C | ASN | 923 | 12.617 | 27.683 | 33.913 | 1.00 | 14.20 |
| ATOM | 1025 | O | ASN | 923 | 11.477 | 28.020 | 34.308 | 1.00 | 12.54 |
| ATOM | 1026 | N | LEU | 924 | 13.037 | 27.832 | 32.668 | 1.00 | 14.23 |
| ATOM | 1027 | H | LEU | 924 | 13.920 | 27.470 | 32.452 | 1.00 | 0.00 |
| ATOM | 1028 | CA | LEU | 924 | 12.260 | 28.534 | 31.671 | 1.00 | 12.20 |
| ATOM | 1029 | CB | LEU | 924 | 13.236 | 28.848 | 30.470 | 1.00 | 12.58 |
| ATOM | 1030 | CG | LEU | 924 | 12.715 | 29.881 | 29.381 | 1.00 | 11.49 |
| ATOM | 1031 | CD1 | LEU | 924 | 12.460 | 31.286 | 30.030 | 1.00 | 10.40 |
| ATOM | 1032 | CD2 | LEU | 924 | 13.756 | 29.974 | 28.232 | 1.00 | 10.52 |
| ATOM | 1033 | C | LEU | 924 | 10.995 | 27.862 | 31.234 | 1.00 | 12.75 |
| ATOM | 1034 | O | LEU | 924 | 10.004 | 28.547 | 30.944 | 1.00 | 13.78 |
| ATOM | 1035 | N | SER | 925 | 10.963 | 26.544 | 31.184 | 1.00 | 10.81 |
| ATOM | 1036 | H | SER | 925 | 11.744 | 26.055 | 31.515 | 1.00 | 0.00 |
| ATOM | 1037 | CA | SER | 925 | 9.723 | 25.850 | 30.762 | 1.00 | 10.73 |
| ATOM | 1038 | CB | SER | 925 | 10.053 | 24.359 | 30.701 | 1.00 | 12.60 |
| ATOM | 1039 | OG | SER | 925 | 8.954 | 23.722 | 30.100 | 1.00 | 16.67 |
| ATOM | 1040 | HG | SER | 925 | 8.739 | 22.944 | 30.635 | 1.00 | 0.00 |
| ATOM | 1041 | C | SER | 925 | 8.569 | 26.124 | 31.747 | 1.00 | 11.36 |
| ATOM | 1042 | O | SER | 925 | 7.441 | 26.587 | 31.487 | 1.00 | 10.42 |
| ATOM | 1043 | N | THR | 926 | 8.860 | 25.873 | 33.011 | 1.00 | 11.52 |
| ATOM | 1044 | H | THR | 926 | 9.683 | 25.389 | 33.252 | 1.00 | 0.00 |
| ATOM | 1045 | CA | THR | 926 | 7.849 | 26.121 | 34.037 | 1.00 | 12.03 |
| ATOM | 1046 | CB | THR | 926 | 8.401 | 25.680 | 35.403 | 1.00 | 10.56 |
| ATOM | 1047 | OG1 | THR | 926 | 8.769 | 24.317 | 35.320 | 1.00 | 10.79 |
| ATOM | 1048 | HG1 | THR | 926 | 8.622 | 23.869 | 36.165 | 1.00 | 0.00 |
| ATOM | 1049 | CG2 | THR | 926 | 7.350 | 25.808 | 36.530 | 1.00 | 12.96 |
| ATOM | 1050 | C | THR | 926 | 7.547 | 27.609 | 34.043 | 1.00 | 13.28 |
| ATOM | 1051 | O | THR | 926 | 6.418 | 28.065 | 34.023 | 1.00 | 13.54 |
| ATOM | 1052 | N | TYR | 927 | 8.560 | 28.443 | 34.035 | 1.00 | 13.11 |
| ATOM | 1053 | H | TYR | 927 | 9.478 | 28.105 | 34.019 | 1.00 | 0.00 |
| ATOM | 1054 | CA | TYR | 927 | 8.289 | 29.899 | 34.068 | 1.00 | 15.88 |
| ATOM | 1055 | CB | TYR | 927 | 9.657 | 30.705 | 34.056 | 1.00 | 14.53 |
| ATOM | 1056 | CG | TYR | 927 | 9.361 | 32.131 | 34.032 | 1.00 | 15.48 |
| ATOM | 1057 | CD1 | TYR | 927 | 9.096 | 32.779 | 35.234 | 1.00 | 14.98 |
| ATOM | 1058 | CE1 | TYR | 927 | 8.786 | 34.136 | 35.210 | 1.00 | 14.78 |
| ATOM | 1059 | CD2 | TYR | 927 | 9.326 | 32.802 | 32.809 | 1.00 | 17.53 |
| ATOM | 1060 | CE2 | TYR | 927 | 9.012 | 34.158 | 32.792 | 1.00 | 18.36 |
| ATOM | 1061 | CZ | TYR | 927 | 8.744 | 34.825 | 34.000 | 1.00 | 16.61 |
| ATOM | 1062 | OH | TYR | 927 | 8.475 | 36.164 | 33.984 | 1.00 | 11.07 |
| ATOM | 1063 | HH | TYR | 927 | 8.610 | 36.518 | 34.870 | 1.00 | 0.00 |
| ATOM | 1064 | C | TYR | 927 | 7.393 | 30.338 | 32.874 | 1.00 | 15.11 |
| ATOM | 1065 | O | TYR | 927 | 6.386 | 30.995 | 33.059 | 1.00 | 17.40 |
| ATOM | 1066 | N | LEU | 928 | 7.641 | 29.992 | 31.627 | 1.00 | 13.75 |
| ATOM | 1067 | H | LEU | 928 | 8.396 | 29.397 | 31.446 | 1.00 | 0.00 |
| ATOM | 1068 | CA | LEU | 928 | 6.754 | 30.478 | 30.514 | 1.00 | 13.93 |
| ATOM | 1069 | CB | LEU | 928 | 7.362 | 30.072 | 29.120 | 1.00 | 8.79 |
| ATOM | 1070 | CG | LEU | 928 | 8.724 | 30.770 | 28.844 | 1.00 | 6.05 |
| ATOM | 1071 | CD1 | LEU | 928 | 9.221 | 30.247 | 27.546 | 1.00 | 5.49 |
| ATOM | 1072 | CD2 | LEU | 928 | 8.628 | 32.324 | 28.868 | 1.00 | 6.04 |
| ATOM | 1073 | C | LEU | 928 | 5.336 | 29.986 | 30.617 | 1.00 | 14.00 |
| ATOM | 1074 | O | LEU | 928 | 4.354 | 30.669 | 30.310 | 1.00 | 14.24 |
| ATOM | 1075 | N | ARG | 929 | 5.212 | 28.741 | 31.028 | 1.00 | 16.59 |
| ATOM | 1076 | H | ARG | 929 | 6.018 | 28.197 | 31.170 | 1.00 | 0.00 |
| ATOM | 1077 | CA | ARG | 929 | 3.872 | 28.123 | 31.174 | 1.00 | 19.97 |
| ATOM | 1078 | CB | ARG | 929 | 4.144 | 26.703 | 31.551 | 1.00 | 20.29 |
| ATOM | 1079 | CG | ARG | 929 | 3.067 | 25.809 | 31.049 | 1.00 | 27.60 |
| ATOM | 1080 | CD | ARG | 929 | 3.775 | 24.453 | 31.129 | 1.00 | 39.93 |
| ATOM | 1081 | NE | ARG | 929 | 4.421 | 24.115 | 32.452 | 1.00 | 46.28 |
| ATOM | 1082 | HE | ARG | 929 | 3.921 | 24.240 | 33.285 | 1.00 | 0.00 |
| ATOM | 1083 | CZ | ARG | 929 | 5.706 | 23.674 | 32.523 | 1.00 | 49.13 |
| ATOM | 1084 | NH1 | ARG | 929 | 6.205 | 23.368 | 33.723 | 1.00 | 50.85 |
| ATOM | 1085 | HH11 | ARG | 929 | 5.644 | 23.469 | 34.543 | 1.00 | 0.00 |
| ATOM | 1086 | HH12 | ARG | 929 | 7.146 | 23.037 | 33.794 | 1.00 | 0.00 |
| ATOM | 1087 | NH2 | ARG | 929 | 6.512 | 23.573 | 31.444 | 1.00 | 47.09 |
| ATOM | 1088 | HH21 | ARG | 929 | 6.183 | 23.831 | 30.534 | 1.00 | 0.00 |
| ATOM | 1089 | HH22 | ARG | 929 | 7.447 | 23.232 | 31.547 | 1.00 | 0.00 |
| ATOM | 1090 | C | ARG | 929 | 3.001 | 28.881 | 32.221 | 1.00 | 21.36 |
| ATOM | 1091 | O | ARG | 929 | 1.764 | 28.868 | 32.163 | 1.00 | 22.82 |
| ATOM | 1092 | N | SER | 930 | 3.673 | 29.574 | 33.190 | 1.00 | 20.93 |
| ATOM | 1093 | H | SER | 930 | 4.655 | 29.536 | 33.175 | 1.00 | 0.00 |
| ATOM | 1094 | CA | SER | 930 | 3.034 | 30.391 | 34.249 | 1.00 | 18.74 |
| ATOM | 1095 | CB | SER | 930 | 3.930 | 30.589 | 35.501 | 1.00 | 16.40 |
| ATOM | 1096 | OG | SER | 930 | 4.980 | 31.514 | 35.253 | 1.00 | 18.58 |
| ATOM | 1097 | HG | SER | 930 | 5.213 | 31.981 | 36.073 | 1.00 | 0.00 |
| ATOM | 1098 | C | SER | 930 | 2.693 | 31.793 | 33.759 | 1.00 | 18.70 |
| ATOM | 1099 | O | SER | 930 | 1.941 | 32.497 | 34.430 | 1.00 | 18.40 |
| ATOM | 1100 | N | LYS | 931 | 3.191 | 32.225 | 32.570 | 1.00 | 16.18 |
| ATOM | 1101 | H | LYS | 931 | 3.854 | 31.653 | 32.125 | 1.00 | 0.00 |
| ATOM | 1102 | CA | LYS | 931 | 2.906 | 33.593 | 32.051 | 1.00 | 14.32 |
| ATOM | 1103 | CB | LYS | 931 | 4.225 | 34.240 | 31.640 | 1.00 | 14.86 |
| ATOM | 1104 | CG | LYS | 931 | 5.309 | 34.226 | 32.704 | 1.00 | 16.57 |
| ATOM | 1105 | CD | LYS | 931 | 4.765 | 34.979 | 33.910 | 1.00 | 19.74 |
| ATOM | 1106 | CE | LYS | 931 | 5.172 | 36.436 | 34.091 | 1.00 | 21.43 |
| ATOM | 1107 | NZ | LYS | 931 | 4.723 | 37.301 | 33.043 | 1.00 | 22.89 |
| ATOM | 1108 | HZ1 | LYS | 931 | 3.693 | 37.203 | 32.941 | 1.00 | 0.00 |
| ATOM | 1109 | HZ2 | LYS | 931 | 5.196 | 37.049 | 32.152 | 1.00 | 0.00 |
| ATOM | 1110 | HZ3 | LYS | 931 | 4.957 | 38.284 | 33.294 | 1.00 | 0.00 |
| ATOM | 1111 | C | LYS | 931 | 1.914 | 33.692 | 30.877 | 1.00 | 15.54 |
| ATOM | 1112 | O | LYS | 931 | 1.762 | 34.712 | 30.182 | 1.00 | 14.51 |
| ATOM | 1113 | N | ARG | 932 | 1.163 | 32.609 | 30.618 | 1.00 | 15.99 |
| ATOM | 1114 | H | ARG | 932 | 1.345 | 31.811 | 31.150 | 1.00 | 0.00 |
| ATOM | 1115 | CA | ARG | 932 | 0.144 | 32.619 | 29.511 | 1.00 | 18.11 |
| ATOM | 1116 | CB | ARG | 932 | −0.536 | 31.208 | 29.452 | 1.00 | 16.54 |
| ATOM | 1117 | CG | ARG | 932 | 0.445 | 30.058 | 29.165 | 1.00 | 16.70 |
| ATOM | 1118 | CD | ARG | 932 | 0.320 | 29.694 | 27.720 | 1.00 | 10.94 |
| ATOM | 1119 | NE | ARG | 932 | 1.258 | 28.610 | 27.603 | 1.00 | 11.52 |
| ATOM | 1120 | HE | ARG | 932 | 2.211 | 28.768 | 27.768 | 1.00 | 0.00 |
| ATOM | 1121 | CZ | ARG | 932 | 0.820 | 27.381 | 27.298 | 1.00 | 14.55 |
| ATOM | 1122 | NH1 | ARG | 932 | 1.702 | 26.382 | 27.173 | 1.00 | 11.67 |
| ATOM | 1123 | HH11 | ARG | 932 | 2.674 | 26.560 | 27.315 | 1.00 | 0.00 |
| ATOM | 1124 | HH12 | ARG | 932 | 1.382 | 25.464 | 26.939 | 1.00 | 0.00 |
| ATOM | 1125 | NH2 | ARG | 932 | −0.474 | 27.114 | 27.096 | 1.00 | 15.30 |
| ATOM | 1126 | HH21 | ARG | 932 | −1.142 | 27.851 | 27.166 | 1.00 | 0.00 |
| ATOM | 1127 | HH22 | ARG | 932 | −0.764 | 26.190 | 26.859 | 1.00 | 0.00 |
| ATOM | 1128 | C | ARG | 932 | −0.949 | 33.757 | 29.641 | 1.00 | 19.02 |
| ATOM | 1129 | O | ARG | 932 | −1.422 | 34.337 | 28.642 | 1.00 | 18.93 |
| ATOM | 1130 | N | ASN | 933 | −1.400 | 34.017 | 30.893 | 1.00 | 18.12 |
| ATOM | 1131 | H | ASN | 933 | −1.013 | 33.499 | 31.635 | 1.00 | 0.00 |
| ATOM | 1132 | CA | ASN | 933 | −2.376 | 35.083 | 31.189 | 1.00 | 19.81 |
| ATOM | 1133 | CB | ASN | 933 | −3.194 | 34.689 | 32.468 | 1.00 | 18.43 |
| ATOM | 1134 | CG | ASN | 933 | −4.521 | 35.504 | 32.551 | 1.00 | 22.85 |
| ATOM | 1135 | OD1 | ASN | 933 | −5.270 | 35.756 | 31.602 | 1.00 | 20.40 |
| ATOM | 1136 | ND2 | ASN | 933 | −4.985 | 35.905 | 33.714 | 1.00 | 22.75 |
| ATOM | 1137 | HD21 | ASN | 933 | −4.563 | 35.735 | 34.576 | 1.00 | 0.00 |
| ATOM | 1138 | HD22 | ASN | 933 | −5.822 | 36.399 | 33.665 | 1.00 | 0.00 |
| ATOM | 1139 | C | ASN | 933 | −1.651 | 36.440 | 31.399 | 1.00 | 20.96 |
| ATOM | 1140 | O | ASN | 933 | −2.251 | 37.455 | 31.775 | 1.00 | 22.58 |
| ATOM | 1141 | N | GLU | 934 | −0.321 | 36.466 | 31.158 | 1.00 | 20.41 |
| ATOM | 1142 | H | GLU | 934 | 0.066 | 35.642 | 30.805 | 1.00 | 0.00 |
| ATOM | 1143 | CA | GLU | 934 | 0.568 | 37.645 | 31.315 | 1.00 | 18.00 |
| ATOM | 1144 | CB | GLU | 934 | 1.339 | 37.439 | 32.563 | 1.00 | 17.64 |
| ATOM | 1145 | CG | GLU | 934 | 0.485 | 37.702 | 33.743 | 1.00 | 20.87 |
| ATOM | 1146 | CD | GLU | 934 | 1.188 | 37.238 | 35.001 | 1.00 | 24.94 |
| ATOM | 1147 | OE1 | GLU | 934 | 0.515 | 36.593 | 35.785 | 1.00 | 32.23 |
| ATOM | 1148 | OE2 | GLU | 934 | 2.353 | 37.509 | 35.255 | 1.00 | 26.83 |
| ATOM | 1149 | C | GLU | 934 | 1.515 | 37.781 | 30.117 | 1.00 | 18.81 |
| ATOM | 1150 | O | GLU | 934 | 2.734 | 37.963 | 30.221 | 1.00 | 20.05 |
| ATOM | 1151 | N | PHE | 935 | 0.906 | 37.685 | 28.932 | 1.00 | 17.41 |
| ATOM | 1152 | H | PHE | 935 | −0.075 | 37.609 | 28.914 | 1.00 | 0.00 |
| ATOM | 1153 | CA | PHE | 935 | 1.607 | 37.785 | 27.676 | 1.00 | 17.54 |
| ATOM | 1154 | CB | PHE | 935 | 1.849 | 36.386 | 27.027 | 1.00 | 13.53 |
| ATOM | 1155 | CG | PHE | 935 | 2.544 | 36.499 | 25.685 | 1.00 | 13.81 |
| ATOM | 1156 | CD1 | PHE | 935 | 1.779 | 36.477 | 24.490 | 1.00 | 11.61 |
| ATOM | 1157 | CD2 | PHE | 935 | 3.952 | 36.569 | 25.607 | 1.00 | 9.55 |
| ATOM | 1158 | CE1 | PHE | 935 | 2.416 | 36.523 | 23.244 | 1.00 | 12.67 |
| ATOM | 1159 | CE2 | PHE | 935 | 4.593 | 36.613 | 24.375 | 1.00 | 10.60 |
| ATOM | 1160 | CZ | PHE | 935 | 3.827 | 36.583 | 23.195 | 1.00 | 12.43 |
| ATOM | 1161 | C | PHE | 935 | 0.860 | 38.624 | 26.676 | 1.00 | 18.25 |
| ATOM | 1162 | O | PHE | 935 | −0.356 | 38.616 | 26.646 | 1.00 | 20.37 |
| ATOM | 1163 | N | VAL | 936 | 1.606 | 39.376 | 25.882 | 1.00 | 18.80 |
| ATOM | 1164 | H | VAL | 936 | 2.558 | 39.466 | 26.104 | 1.00 | 0.00 |
| ATOM | 1165 | CA | VAL | 936 | 1.049 | 40.197 | 24.818 | 1.00 | 20.33 |
| ATOM | 1166 | CB | VAL | 936 | 0.758 | 41.689 | 25.111 | 1.00 | 25.79 |
| ATOM | 1167 | CG1 | VAL | 936 | −0.740 | 41.613 | 24.970 | 1.00 | 26.82 |
| ATOM | 1168 | CG2 | VAL | 936 | 1.454 | 42.376 | 26.329 | 1.00 | 24.06 |
| ATOM | 1169 | C | VAL | 936 | 2.107 | 40.311 | 23.767 | 1.00 | 19.26 |
| ATOM | 1170 | O | VAL | 936 | 3.283 | 40.427 | 24.096 | 1.00 | 17.94 |
| ATOM | 1171 | N | PRO | 937 | 1.753 | 40.279 | 22.497 | 1.00 | 20.85 |
| ATOM | 1172 | CD | PRO | 937 | 0.407 | 40.108 | 21.901 | 1.00 | 21.81 |
| ATOM | 1173 | CA | PRO | 937 | 2.815 | 40.170 | 21.494 | 1.00 | 22.91 |
| ATOM | 1174 | CB | PRO | 937 | 2.110 | 39.918 | 20.166 | 1.00 | 21.52 |
| ATOM | 1175 | CG | PRO | 937 | 0.814 | 39.279 | 20.657 | 1.00 | 21.81 |
| ATOM | 1176 | C | PRO | 937 | 3.706 | 41.378 | 21.453 | 1.00 | 26.52 |
| ATOM | 1177 | O | PRO | 937 | 4.931 | 41.301 | 21.445 | 1.00 | 28.22 |
| ATOM | 1178 | N | TYR | 938 | 3.099 | 42.519 | 21.545 | 1.00 | 29.94 |
| ATOM | 1179 | H | TYR | 938 | 2.171 | 42.552 | 21.853 | 1.00 | 0.00 |
| ATOM | 1180 | CA | TYR | 938 | 3.782 | 43.820 | 21.477 | 1.00 | 31.85 |
| ATOM | 1181 | CB | TYR | 938 | 3.517 | 44.301 | 20.016 | 1.00 | 32.90 |
| ATOM | 1182 | C | TYR | 938 | 3.056 | 44.471 | 22.635 | 1.00 | 34.35 |
| ATOM | 1183 | O | TYR | 938 | 1.810 | 44.373 | 22.715 | 1.00 | 36.12 |
| ATOM | 1185 | CB | ASP | 998 | 6.074 | 42.226 | 35.380 | 1.00 | 30.69 |
| ATOM | 1186 | C | ASP | 998 | 4.803 | 41.587 | 33.226 | 1.00 | 30.65 |
| ATOM | 1187 | O | ASP | 998 | 5.098 | 40.405 | 33.411 | 1.00 | 29.91 |
| ATOM | 1188 | HT1 | ASP | 998 | 3.305 | 42.075 | 35.385 | 1.00 | 0.00 |
| ATOM | 1189 | HT2 | ASP | 998 | 2.988 | 43.410 | 34.395 | 1.00 | 0.00 |
| ATOM | 1190 | N | ASP | 998 | 3.693 | 42.975 | 35.025 | 1.00 | 32.81 |
| ATOM | 1191 | HT3 | ASP | 998 | 3.859 | 43.609 | 35.838 | 1.00 | 0.00 |
| ATOM | 1192 | CA | ASP | 998 | 4.988 | 42.684 | 34.319 | 1.00 | 31.83 |
| ATOM | 1193 | N | PHE | 999 | 4.142 | 41.980 | 32.126 | 1.00 | 27.73 |
| ATOM | 1194 | H | PHE | 999 | 4.011 | 42.927 | 31.923 | 1.00 | 0.00 |
| ATOM | 1195 | CA | PHE | 999 | 3.884 | 41.046 | 31.028 | 1.00 | 25.58 |
| ATOM | 1196 | CB | PHE | 999 | 2.873 | 41.657 | 30.038 | 1.00 | 26.61 |
| ATOM | 1197 | CG | PHE | 999 | 1.462 | 41.418 | 30.429 | 1.00 | 29.93 |
| ATOM | 1198 | CD1 | PHE | 999 | 0.539 | 41.066 | 29.406 | 1.00 | 30.62 |
| ATOM | 1199 | CD2 | PHE | 999 | 1.024 | 41.567 | 31.773 | 1.00 | 29.73 |
| ATOM | 1200 | CE1 | PHE | 999 | −0.826 | 40.848 | 29.669 | 1.00 | 29.83 |
| ATOM | 1201 | CE2 | PHE | 999 | −0.328 | 41.359 | 32.057 | 1.00 | 30.39 |
| ATOM | 1202 | CZ | PHE | 999 | −1.226 | 41.003 | 31.009 | 1.00 | 32.11 |
| ATOM | 1203 | C | PHE | 999 | 5.079 | 40.612 | 30.208 | 1.00 | 24.67 |
| ATOM | 1204 | O | PHE | 999 | 6.094 | 41.304 | 30.071 | 1.00 | 25.41 |
| ATOM | 1205 | N | LEU | 1000 | 5.008 | 39.412 | 29.703 | 1.00 | 22.25 |
| ATOM | 1206 | H | LEU | 1000 | 4.316 | 38.800 | 30.001 | 1.00 | 0.00 |
| ATOM | 1207 | CA | LEU | 1000 | 6.051 | 38.971 | 28.787 | 1.00 | 17.43 |
| ATOM | 1208 | CB | LEU | 1000 | 6.128 | 37.452 | 28.658 | 1.00 | 16.97 |
| ATOM | 1209 | CG | LEU | 1000 | 6.921 | 36.612 | 29.585 | 1.00 | 14.78 |
| ATOM | 1210 | CD1 | LEU | 1000 | 6.636 | 35.222 | 29.126 | 1.00 | 15.53 |
| ATOM | 1211 | CD2 | LEU | 1000 | 8.430 | 36.891 | 29.555 | 1.00 | 17.96 |
| ATOM | 1212 | C | LEU | 1000 | 5.540 | 39.493 | 27.443 | 1.00 | 16.93 |
| ATOM | 1213 | O | LEU | 1000 | 4.333 | 39.721 | 27.248 | 1.00 | 16.01 |
| ATOM | 1214 | N | THR | 1001 | 6.415 | 39.639 | 26.462 | 1.00 | 15.39 |
| ATOM | 1215 | H | THR | 1001 | 7.344 | 39.391 | 26.616 | 1.00 | 0.00 |
| ATOM | 1216 | CA | THR | 1001 | 5.998 | 40.112 | 25.118 | 1.00 | 16.05 |
| ATOM | 1217 | CB | THR | 1001 | 6.240 | 41.641 | 24.874 | 1.00 | 17.01 |
| ATOM | 1218 | OG1 | THR | 1001 | 7.651 | 41.825 | 24.971 | 1.00 | 19.97 |
| ATOM | 1219 | HG1 | THR | 1001 | 7.799 | 42.738 | 25.249 | 1.00 | 0.00 |
| ATOM | 1220 | CG2 | THR | 1001 | 5.574 | 42.583 | 25.870 | 1.00 | 14.66 |
| ATOM | 1221 | C | THR | 1001 | 6.855 | 39.394 | 24.114 | 1.00 | 16.20 |
| ATOM | 1222 | O | THR | 1001 | 7.869 | 38.787 | 24.493 | 1.00 | 15.90 |
| ATOM | 1223 | N | LEU | 1002 | 6.561 | 39.474 | 22.811 | 1.00 | 16.72 |
| ATOM | 1224 | H | LEU | 1002 | 5.771 | 39.992 | 22.543 | 1.00 | 0.00 |
| ATOM | 1225 | CA | LEU | 1002 | 7.391 | 38.782 | 21.788 | 1.00 | 17.23 |
| ATOM | 1226 | CB | LEU | 1002 | 6.794 | 39.151 | 20.436 | 1.00 | 16.16 |
| ATOM | 1227 | CG | LEU | 1002 | 7.301 | 38.393 | 19.214 | 1.00 | 16.93 |
| ATOM | 1228 | CD1 | LEU | 1002 | 7.262 | 36.847 | 19.332 | 1.00 | 11.65 |
| ATOM | 1229 | CD2 | LEU | 1002 | 6.403 | 38.921 | 18.071 | 1.00 | 15.62 |
| ATOM | 1230 | C | LEU | 1002 | 8.881 | 39.145 | 21.893 | 1.00 | 18.76 |
| ATOM | 1231 | O | LEU | 1002 | 9.804 | 38.343 | 21.674 | 1.00 | 18.82 |
| ATOM | 1232 | N | GLU | 1003 | 9.135 | 40.399 | 22.279 | 1.00 | 18.56 |
| ATOM | 1233 | H | GLU | 1003 | 8.393 | 41.037 | 22.309 | 1.00 | 0.00 |
| ATOM | 1234 | CA | GLU | 1003 | 10.499 | 40.870 | 22.462 | 1.00 | 17.00 |
| ATOM | 1235 | CB | GLU | 1003 | 10.489 | 42.234 | 22.987 | 1.00 | 20.27 |
| ATOM | 1236 | CG | GLU | 1003 | 11.584 | 42.852 | 22.175 | 1.00 | 24.86 |
| ATOM | 1237 | CD | GLU | 1003 | 12.120 | 44.035 | 22.902 | 1.00 | 29.23 |
| ATOM | 1238 | OE1 | GLU | 1003 | 11.378 | 44.749 | 23.591 | 1.00 | 30.66 |
| ATOM | 1239 | OE2 | GLU | 1003 | 13.316 | 44.229 | 22.756 | 1.00 | 32.63 |
| ATOM | 1240 | C | GLU | 1003 | 11.253 | 40.045 | 23.460 | 1.00 | 15.42 |
| ATOM | 1241 | O | GLU | 1003 | 12.394 | 39.671 | 23.261 | 1.00 | 16.49 |
| ATOM | 1242 | N | HIS | 1004 | 10.687 | 39.836 | 24.628 | 1.00 | 15.02 |
| ATOM | 1243 | H | HIS | 1004 | 9.787 | 40.201 | 24.756 | 1.00 | 0.00 |
| ATOM | 1244 | CA | HIS | 1004 | 11.300 | 39.003 | 25.674 | 1.00 | 14.20 |
| ATOM | 1245 | C | HIS | 1004 | 11.670 | 37.624 | 25.117 | 1.00 | 13.88 |
| ATOM | 1246 | O | HIS | 1004 | 12.738 | 37.102 | 25.404 | 1.00 | 14.33 |
| ATOM | 1247 | CB | HIS | 1004 | 10.345 | 38.666 | 26.809 | 1.00 | 12.47 |
| ATOM | 1248 | CG | HIS | 1004 | 10.031 | 39.874 | 27.518 | 1.00 | 12.45 |
| ATOM | 1249 | ND1 | HIS | 1004 | 10.558 | 40.211 | 28.720 | 1.00 | 16.18 |
| ATOM | 1250 | HD1 | HIS | 1004 | 11.116 | 39.638 | 29.290 | 1.00 | 0.00 |
| ATOM | 1251 | CD2 | HIS | 1004 | 9.171 | 40.844 | 27.109 | 1.00 | 8.88 |
| ATOM | 1252 | NE2 | HIS | 1004 | 9.195 | 41.747 | 28.088 | 1.00 | 15.41 |
| ATOM | 1253 | CE1 | HIS | 1004 | 10.036 | 41.390 | 29.102 | 1.00 | 11.02 |
| ATOM | 1254 | N | LEU | 1005 | 10.735 | 36.999 | 24.385 | 1.00 | 12.64 |
| ATOM | 1255 | H | LEU | 1005 | 9.896 | 37.487 | 24.250 | 1.00 | 0.00 |
| ATOM | 1256 | CA | LEU | 1005 | 10.864 | 35.634 | 23.780 | 1.00 | 11.43 |
| ATOM | 1257 | CB | LEU | 1005 | 9.487 | 35.271 | 23.159 | 1.00 | 10.84 |
| ATOM | 1258 | CG | LEU | 1005 | 8.408 | 34.561 | 24.011 | 1.00 | 10.35 |
| ATOM | 1259 | CD1 | LEU | 1005 | 8.370 | 35.014 | 25.425 | 1.00 | 10.63 |
| ATOM | 1260 | CD2 | LEU | 1005 | 7.151 | 34.657 | 23.221 | 1.00 | 7.70 |
| ATOM | 1261 | C | LEU | 1005 | 11.996 | 35.494 | 22.765 | 1.00 | 10.78 |
| ATOM | 1262 | O | LEU | 1005 | 12.726 | 34.486 | 22.779 | 1.00 | 9.24 |
| ATOM | 1263 | N | ILE | 1006 | 12.143 | 36.483 | 21.866 | 1.00 | 10.52 |
| ATOM | 1264 | H | ILE | 1006 | 11.440 | 37.173 | 21.857 | 1.00 | 0.00 |
| ATOM | 1265 | CA | ILE | 1006 | 13.252 | 36.522 | 20.868 | 1.00 | 10.50 |
| ATOM | 1266 | CB | ILE | 1006 | 13.080 | 37.642 | 19.801 | 1.00 | 10.87 |
| ATOM | 1267 | CG2 | ILE | 1006 | 14.234 | 37.597 | 18.738 | 1.00 | 8.71 |
| ATOM | 1268 | CG1 | ILE | 1006 | 11.685 | 37.441 | 19.160 | 1.00 | 13.09 |
| ATOM | 1269 | CD1 | ILE | 1006 | 11.248 | 38.414 | 18.009 | 1.00 | 15.22 |
| ATOM | 1270 | C | ILE | 1006 | 14.557 | 36.799 | 21.589 | 1.00 | 10.59 |
| ATOM | 1271 | O | ILE | 1006 | 15.629 | 36.334 | 21.241 | 1.00 | 14.26 |
| ATOM | 1272 | N | CYS | 1007 | 14.519 | 37.695 | 22.545 | 1.00 | 10.11 |
| ATOM | 1273 | H | CYS | 1007 | 13.688 | 38.207 | 22.648 | 1.00 | 0.00 |
| ATOM | 1274 | CA | CYS | 1007 | 15.662 | 38.020 | 23.371 | 1.00 | 9.38 |
| ATOM | 1275 | CB | CYS | 1007 | 15.265 | 39.142 | 24.380 | 1.00 | 10.19 |
| ATOM | 1276 | SG | CYS | 1007 | 16.707 | 39.479 | 25.392 | 1.00 | 17.87 |
| ATOM | 1277 | C | CYS | 1007 | 16.120 | 36.753 | 24.125 | 1.00 | 9.24 |
| ATOM | 1278 | O | CYS | 1007 | 17.295 | 36.433 | 24.138 | 1.00 | 9.63 |
| ATOM | 1279 | N | TYR | 1008 | 15.270 | 35.953 | 24.793 | 1.00 | 10.22 |
| ATOM | 1280 | H | TYR | 1008 | 14.336 | 36.233 | 24.872 | 1.00 | 0.00 |
| ATOM | 1281 | CA | TYR | 1008 | 15.728 | 34.722 | 25.491 | 1.00 | 7.90 |
| ATOM | 1282 | CB | TYR | 1008 | 14.512 | 34.055 | 26.129 | 1.00 | 8.86 |
| ATOM | 1283 | CG | TYR | 1008 | 13.848 | 34.829 | 27.183 | 1.00 | 9.99 |
| ATOM | 1284 | CD1 | TYR | 1008 | 12.472 | 34.531 | 27.391 | 1.00 | 13.26 |
| ATOM | 1285 | CE1 | TYR | 1008 | 11.766 | 35.186 | 28.432 | 1.00 | 11.32 |
| ATOM | 1286 | CD2 | TYR | 1008 | 14.519 | 35.780 | 27.988 | 1.00 | 13.91 |
| ATOM | 1287 | CE2 | TYR | 1008 | 13.818 | 36.443 | 29.029 | 1.00 | 11.83 |
| ATOM | 1288 | CZ | TYR | 1008 | 12.459 | 36.115 | 29.217 | 1.00 | 13.11 |
| ATOM | 1289 | OH | TYR | 1008 | 11.789 | 36.611 | 30.280 | 1.00 | 13.52 |
| ATOM | 1290 | HH | TYR | 1008 | 12.340 | 37.242 | 30.757 | 1.00 | 0.00 |
| ATOM | 1291 | C | TYR | 1008 | 16.380 | 33.747 | 24.507 | 1.00 | 7.08 |
| ATOM | 1292 | O | TYR | 1008 | 17.402 | 33.094 | 24.767 | 1.00 | 7.75 |
| ATOM | 1293 | N | SER | 1009 | 15.768 | 33.582 | 23.331 | 1.00 | 7.50 |
| ATOM | 1294 | H | SER | 1009 | 14.858 | 33.938 | 23.245 | 1.00 | 0.00 |
| ATOM | 1295 | CA | SER | 1009 | 16.273 | 32.692 | 22.225 | 1.00 | 7.86 |
| ATOM | 1296 | CB | SER | 1009 | 15.342 | 32.802 | 20.981 | 1.00 | 6.63 |
| ATOM | 1297 | OG | SER | 1009 | 14.004 | 32.474 | 21.292 | 1.00 | 10.44 |
| ATOM | 1298 | HG | SER | 1009 | 13.409 | 32.744 | 20.591 | 1.00 | 0.00 |
| ATOM | 1299 | C | SER | 1009 | 17.678 | 33.074 | 21.799 | 1.00 | 6.60 |
| ATOM | 1300 | O | SER | 1009 | 18.581 | 32.249 | 21.622 | 1.00 | 9.02 |
| ATOM | 1301 | N | PHE | 1010 | 17.850 | 34.357 | 21.485 | 1.00 | 7.20 |
| ATOM | 1302 | H | PHE | 1010 | 17.051 | 34.929 | 21.495 | 1.00 | 0.00 |
| ATOM | 1303 | CA | PHE | 1010 | 19.150 | 34.974 | 21.097 | 1.00 | 7.36 |
| ATOM | 1304 | CB | PHE | 1010 | 18.939 | 36.549 | 20.950 | 1.00 | 7.82 |
| ATOM | 1305 | CG | PHE | 1010 | 20.232 | 37.286 | 20.834 | 1.00 | 11.24 |
| ATOM | 1306 | CD1 | PHE | 1010 | 20.624 | 38.185 | 21.844 | 1.00 | 13.22 |
| ATOM | 1307 | CD2 | PHE | 1010 | 21.107 | 37.091 | 19.741 | 1.00 | 9.87 |
| ATOM | 1308 | CE1 | PHE | 1010 | 21.863 | 38.866 | 21.766 | 1.00 | 13.93 |
| ATOM | 1309 | CE2 | PHE | 1010 | 22.343 | 37.755 | 19.638 | 1.00 | 10.68 |
| ATOM | 1310 | CZ | PHE | 1010 | 22.713 | 38.636 | 20.659 | 1.00 | 13.80 |
| ATOM | 1311 | C | PHE | 1010 | 20.206 | 34.661 | 22.201 | 1.00 | 8.98 |
| ATOM | 1312 | O | PHE | 1010 | 21.372 | 34.281 | 21.919 | 1.00 | 9.84 |
| ATOM | 1313 | N | GLN | 1011 | 19.833 | 34.902 | 23.499 | 1.00 | 9.42 |
| ATOM | 1314 | H | GLN | 1011 | 18.966 | 35.336 | 23.658 | 1.00 | 0.00 |
| ATOM | 1315 | CA | GLN | 1011 | 20.711 | 34.636 | 24.649 | 1.00 | 6.78 |
| ATOM | 1316 | CB | GLN | 1011 | 20.100 | 35.033 | 25.932 | 1.00 | 7.27 |
| ATOM | 1317 | CG | GLN | 1011 | 20.015 | 36.508 | 26.108 | 1.00 | 9.45 |
| ATOM | 1318 | CD | GLN | 1011 | 19.405 | 36.763 | 27.496 | 1.00 | 12.64 |
| ATOM | 1319 | OE1 | GLN | 1011 | 20.079 | 36.615 | 28.511 | 1.00 | 8.63 |
| ATOM | 1320 | NE2 | GLN | 1011 | 18.135 | 37.177 | 27.609 | 1.00 | 11.08 |
| ATOM | 1321 | HE21 | GLN | 1011 | 17.587 | 37.322 | 26.820 | 1.00 | 0.00 |
| ATOM | 1322 | HE22 | GLN | 1011 | 17.788 | 37.332 | 28.506 | 1.00 | 0.00 |
| ATOM | 1323 | C | GLN | 1011 | 21.023 | 33.165 | 24.777 | 1.00 | 8.57 |
| ATOM | 1324 | O | GLN | 1011 | 22.156 | 32.816 | 25.084 | 1.00 | 11.00 |
| ATOM | 1325 | N | VAL | 1012 | 20.084 | 32.207 | 24.590 | 1.00 | 9.46 |
| ATOM | 1326 | H | VAL | 1012 | 19.154 | 32.466 | 24.464 | 1.00 | 0.00 |
| ATOM | 1327 | CA | VAL | 1012 | 20.510 | 30.784 | 24.674 | 1.00 | 7.97 |
| ATOM | 1328 | CB | VAL | 1012 | 19.304 | 29.887 | 24.602 | 1.00 | 7.75 |
| ATOM | 1329 | CG1 | VAL | 1012 | 19.767 | 28.394 | 24.695 | 1.00 | 5.22 |
| ATOM | 1330 | CG2 | VAL | 1012 | 18.334 | 30.272 | 25.738 | 1.00 | 7.35 |
| ATOM | 1331 | C | VAL | 1012 | 21.477 | 30.497 | 23.497 | 1.00 | 7.31 |
| ATOM | 1332 | O | VAL | 1012 | 22.477 | 29.812 | 23.670 | 1.00 | 9.28 |
| ATOM | 1333 | N | ALA | 1013 | 21.220 | 30.943 | 22.263 | 1.00 | 7.40 |
| ATOM | 1334 | H | ALA | 1013 | 20.356 | 31.393 | 22.121 | 1.00 | 0.00 |
| ATOM | 1335 | CA | ALA | 1013 | 22.152 | 30.736 | 21.133 | 1.00 | 7.15 |
| ATOM | 1336 | CB | ALA | 1013 | 21.471 | 31.339 | 19.846 | 1.00 | 8.57 |
| ATOM | 1337 | C | ALA | 1013 | 23.541 | 31.345 | 21.447 | 1.00 | 8.61 |
| ATOM | 1338 | O | ALA | 1013 | 24.573 | 30.763 | 21.111 | 1.00 | 9.63 |
| ATOM | 1339 | N | LYS | 1014 | 23.667 | 32.506 | 22.115 | 1.00 | 9.80 |
| ATOM | 1340 | H | LYS | 1014 | 22.861 | 33.046 | 22.230 | 1.00 | 0.00 |
| ATOM | 1341 | CA | LYS | 1014 | 24.980 | 33.075 | 22.527 | 1.00 | 8.82 |
| ATOM | 1342 | CB | LYS | 1014 | 24.857 | 34.432 | 23.307 | 1.00 | 4.76 |
| ATOM | 1343 | CG | LYS | 1014 | 24.492 | 35.662 | 22.521 | 1.00 | 10.15 |
| ATOM | 1344 | CD | LYS | 1014 | 25.580 | 36.133 | 21.536 | 1.00 | 8.77 |
| ATOM | 1345 | CE | LYS | 1014 | 26.426 | 37.116 | 22.296 | 1.00 | 12.27 |
| ATOM | 1346 | NZ | LYS | 1014 | 27.391 | 37.682 | 21.356 | 1.00 | 12.47 |
| ATOM | 1347 | HZ1 | LYS | 1014 | 26.925 | 37.952 | 20.472 | 1.00 | 0.00 |
| ATOM | 1348 | HZ2 | LYS | 1014 | 28.103 | 36.947 | 21.175 | 1.00 | 0.00 |
| ATOM | 1349 | HZ3 | LYS | 1014 | 27.842 | 38.525 | 21.764 | 1.00 | 0.00 |
| ATOM | 1350 | C | LYS | 1014 | 25.701 | 32.112 | 23.512 | 1.00 | 8.17 |
| ATOM | 1351 | O | LYS | 1014 | 26.922 | 31.884 | 23.511 | 1.00 | 8.92 |
| ATOM | 1352 | N | GLY | 1015 | 24.980 | 31.624 | 24.515 | 1.00 | 8.12 |
| ATOM | 1353 | H | GLY | 1015 | 24.067 | 31.950 | 24.628 | 1.00 | 0.00 |
| ATOM | 1354 | CA | GLY | 1015 | 25.530 | 30.658 | 25.530 | 1.00 | 6.16 |
| ATOM | 1355 | C | GLY | 1015 | 26.000 | 29.370 | 24.868 | 1.00 | 6.55 |
| ATOM | 1356 | O | GLY | 1015 | 27.006 | 28.811 | 25.291 | 1.00 | 6.23 |
| ATOM | 1357 | N | MET | 1016 | 25.234 | 28.828 | 23.878 | 1.00 | 6.05 |
| ATOM | 1358 | H | MET | 1016 | 24.365 | 29.251 | 23.714 | 1.00 | 0.00 |
| ATOM | 1359 | CA | MET | 1016 | 25.599 | 27.609 | 23.137 | 1.00 | 6.47 |
| ATOM | 1360 | CB | MET | 1016 | 24.391 | 27.159 | 22.386 | 1.00 | 7.33 |
| ATOM | 1361 | CG | MET | 1016 | 23.307 | 26.401 | 23.213 | 1.00 | 8.25 |
| ATOM | 1362 | SD | MET | 1016 | 23.948 | 25.065 | 24.223 | 1.00 | 11.01 |
| ATOM | 1363 | CE | MET | 1016 | 24.843 | 23.988 | 23.068 | 1.00 | 5.34 |
| ATOM | 1364 | C | MET | 1016 | 26.830 | 27.858 | 22.193 | 1.00 | 10.04 |
| ATOM | 1365 | O | MET | 1016 | 27.727 | 27.019 | 22.024 | 1.00 | 10.42 |
| ATOM | 1366 | N | GLU | 1017 | 26.892 | 29.034 | 21.545 | 1.00 | 9.25 |
| ATOM | 1367 | H | GLU | 1017 | 26.091 | 29.590 | 21.521 | 1.00 | 0.00 |
| ATOM | 1368 | CA | GLU | 1017 | 28.074 | 29.424 | 20.769 | 1.00 | 9.98 |
| ATOM | 1369 | CB | GLU | 1017 | 27.835 | 30.787 | 20.207 | 1.00 | 6.93 |
| ATOM | 1370 | CG | GLU | 1017 | 29.029 | 31.259 | 19.417 | 1.00 | 7.54 |
| ATOM | 1371 | CD | GLU | 1017 | 28.761 | 32.683 | 18.981 | 1.00 | 13.73 |
| ATOM | 1372 | OE1 | GLU | 1017 | 28.344 | 33.449 | 19.852 | 1.00 | 15.46 |
| ATOM | 1373 | OE2 | GLU | 1017 | 28.975 | 33.021 | 17.803 | 1.00 | 16.01 |
| ATOM | 1374 | C | GLU | 1017 | 29.235 | 29.443 | 21.735 | 1.00 | 10.45 |
| ATOM | 1375 | O | GLU | 1017 | 30.322 | 28.939 | 21.453 | 1.00 | 14.15 |
| ATOM | 1376 | N | PHE | 1018 | 29.103 | 30.033 | 22.926 | 1.00 | 10.58 |
| ATOM | 1377 | H | PHE | 1018 | 28.327 | 30.607 | 23.091 | 1.00 | 0.00 |
| ATOM | 1378 | CA | PHE | 1018 | 30.214 | 29.990 | 23.916 | 1.00 | 10.61 |
| ATOM | 1379 | CB | PHE | 1018 | 29.806 | 30.731 | 25.251 | 1.00 | 11.68 |
| ATOM | 1380 | CG | PHE | 1018 | 30.803 | 30.673 | 26.385 | 1.00 | 13.50 |
| ATOM | 1381 | CD1 | PHE | 1018 | 30.573 | 29.896 | 27.541 | 1.00 | 14.72 |
| ATOM | 1382 | CD2 | PHE | 1018 | 32.003 | 31.403 | 26.306 | 1.00 | 13.75 |
| ATOM | 1383 | CE1 | PHE | 1018 | 31.544 | 29.841 | 28.587 | 1.00 | 15.11 |
| ATOM | 1384 | CE2 | PHE | 1018 | 32.951 | 31.345 | 27.342 | 1.00 | 13.21 |
| ATOM | 1385 | CZ | PHE | 1018 | 32.733 | 30.560 | 28.489 | 1.00 | 11.31 |
| ATOM | 1386 | C | PHE | 1018 | 30.566 | 28.528 | 24.253 | 1.00 | 12.59 |
| ATOM | 1387 | O | PHE | 1018 | 31.731 | 28.111 | 24.241 | 1.00 | 11.76 |
| ATOM | 1388 | N | LEU | 1019 | 29.569 | 27.683 | 24.610 | 1.00 | 12.49 |
| ATOM | 1389 | H | LEU | 1019 | 28.679 | 28.069 | 24.758 | 1.00 | 0.00 |
| ATOM | 1390 | CA | LEU | 1019 | 29.837 | 26.268 | 24.928 | 1.00 | 12.04 |
| ATOM | 1391 | CB | LEU | 1019 | 28.503 | 25.467 | 25.129 | 1.00 | 14.29 |
| ATOM | 1392 | CG | LEU | 1019 | 28.067 | 25.380 | 26.613 | 1.00 | 24.43 |
| ATOM | 1393 | CD1 | LEU | 1019 | 26.724 | 24.586 | 26.856 | 1.00 | 23.67 |
| ATOM | 1394 | CD2 | LEU | 1019 | 29.258 | 24.683 | 27.354 | 1.00 | 23.40 |
| ATOM | 1395 | C | LEU | 1019 | 30.657 | 25.610 | 23.804 | 1.00 | 12.02 |
| ATOM | 1396 | O | LEU | 1019 | 31.676 | 24.946 | 24.011 | 1.00 | 9.68 |
| ATOM | 1397 | N | ALA | 1020 | 30.182 | 25.745 | 22.576 | 1.00 | 12.41 |
| ATOM | 1398 | H | ALA | 1020 | 29.344 | 26.223 | 22.433 | 1.00 | 0.00 |
| ATOM | 1399 | CA | ALA | 1020 | 30.897 | 25.160 | 21.427 | 1.00 | 11.83 |
| ATOM | 1400 | CB | ALA | 1020 | 30.124 | 25.387 | 20.119 | 1.00 | 9.86 |
| ATOM | 1401 | C | ALA | 1020 | 32.265 | 25.820 | 21.316 | 1.00 | 12.14 |
| ATOM | 1402 | O | ALA | 1020 | 33.227 | 25.183 | 20.874 | 1.00 | 14.46 |
| ATOM | 1403 | N | SER | 1021 | 32.454 | 27.082 | 21.638 | 1.00 | 12.21 |
| ATOM | 1404 | H | SER | 1021 | 31.728 | 27.619 | 22.014 | 1.00 | 0.00 |
| ATOM | 1405 | CA | SER | 1021 | 33.844 | 27.644 | 21.537 | 1.00 | 13.00 |
| ATOM | 1406 | CB | SER | 1021 | 33.848 | 29.168 | 21.770 | 1.00 | 14.17 |
| ATOM | 1407 | OG | SER | 1021 | 33.863 | 29.519 | 23.172 | 1.00 | 15.03 |
| ATOM | 1408 | HG | SER | 1021 | 34.069 | 30.465 | 23.266 | 1.00 | 0.00 |
| ATOM | 1409 | C | SER | 1021 | 34.834 | 26.984 | 22.565 | 1.00 | 15.38 |
| ATOM | 1410 | O | SER | 1021 | 36.059 | 27.023 | 22.414 | 1.00 | 16.31 |
| ATOM | 1411 | N | ARG | 1022 | 34.354 | 26.352 | 23.653 | 1.00 | 14.41 |
| ATOM | 1412 | H | ARG | 1022 | 33.400 | 26.474 | 23.820 | 1.00 | 0.00 |
| ATOM | 1413 | CA | ARG | 1022 | 35.174 | 25.634 | 24.717 | 1.00 | 14.47 |
| ATOM | 1414 | CB | ARG | 1022 | 34.526 | 25.616 | 26.135 | 1.00 | 13.55 |
| ATOM | 1415 | CG | ARG | 1022 | 33.830 | 26.915 | 26.417 | 1.00 | 14.19 |
| ATOM | 1416 | CD | ARG | 1022 | 34.773 | 27.895 | 27.059 | 1.00 | 18.15 |
| ATOM | 1417 | NE | ARG | 1022 | 35.940 | 28.140 | 26.312 | 1.00 | 15.98 |
| ATOM | 1418 | HE | ARG | 1022 | 35.796 | 28.475 | 25.403 | 1.00 | 0.00 |
| ATOM | 1419 | CZ | ARG | 1022 | 37.208 | 27.948 | 26.653 | 1.00 | 19.02 |
| ATOM | 1420 | NH1 | ARG | 1022 | 38.107 | 28.259 | 25.712 | 1.00 | 19.71 |
| ATOM | 1421 | HH11 | ARG | 1022 | 37.823 | 28.597 | 24.815 | 1.00 | 0.00 |
| ATOM | 1422 | HH12 | ARG | 1022 | 39.076 | 28.137 | 25.924 | 1.00 | 0.00 |
| ATOM | 1423 | NH2 | ARG | 1022 | 37.663 | 27.442 | 27.782 | 1.00 | 21.63 |
| ATOM | 1424 | HH21 | ARG | 1022 | 36.978 | 27.175 | 28.459 | 1.00 | 0.00 |
| ATOM | 1425 | HH22 | ARG | 1022 | 38.636 | 27.321 | 27.974 | 1.00 | 0.00 |
| ATOM | 1426 | C | ARG | 1022 | 35.324 | 24.159 | 24.392 | 1.00 | 14.47 |
| ATOM | 1427 | O | ARG | 1022 | 35.660 | 23.326 | 25.226 | 1.00 | 12.99 |
| ATOM | 1428 | N | LYS | 1023 | 34.825 | 23.830 | 23.222 | 1.00 | 15.33 |
| ATOM | 1429 | H | LYS | 1023 | 34.432 | 24.554 | 22.703 | 1.00 | 0.00 |
| ATOM | 1430 | CA | LYS | 1023 | 34.779 | 22.508 | 22.600 | 1.00 | 17.89 |
| ATOM | 1431 | CB | LYS | 1023 | 36.250 | 22.065 | 22.406 | 1.00 | 22.81 |
| ATOM | 1432 | CG | LYS | 1023 | 36.911 | 23.031 | 21.423 | 1.00 | 26.00 |
| ATOM | 1433 | CD | LYS | 1023 | 38.377 | 22.666 | 21.434 | 1.00 | 35.48 |
| ATOM | 1434 | CE | LYS | 1023 | 39.202 | 23.659 | 20.544 | 1.00 | 43.51 |
| ATOM | 1435 | NZ | LYS | 1023 | 38.810 | 23.652 | 19.099 | 1.00 | 46.80 |
| ATOM | 1436 | HZ1 | LYS | 1023 | 37.773 | 23.698 | 19.023 | 1.00 | 0.00 |
| ATOM | 1437 | HZ2 | LYS | 1023 | 39.168 | 22.802 | 18.626 | 1.00 | 0.00 |
| ATOM | 1438 | HZ3 | LYS | 1023 | 39.220 | 24.499 | 18.654 | 1.00 | 0.00 |
| ATOM | 1439 | C | LYS | 1023 | 33.934 | 21.502 | 23.397 | 1.00 | 16.83 |
| ATOM | 1440 | O | LYS | 1023 | 34.199 | 20.287 | 23.557 | 1.00 | 12.62 |
| ATOM | 1441 | N | CYS | 1024 | 32.834 | 22.050 | 23.915 | 1.00 | 15.46 |
| ATOM | 1442 | H | CYS | 1024 | 32.601 | 22.986 | 23.708 | 1.00 | 0.00 |
| ATOM | 1443 | CA | CYS | 1024 | 31.918 | 21.199 | 24.675 | 1.00 | 14.09 |
| ATOM | 1444 | CB | CYS | 1024 | 31.698 | 21.753 | 26.113 | 1.00 | 12.39 |
| ATOM | 1445 | SG | CYS | 1024 | 33.236 | 21.778 | 27.054 | 1.00 | 14.57 |
| ATOM | 1446 | C | CYS | 1024 | 30.612 | 21.124 | 23.967 | 1.00 | 13.46 |
| ATOM | 1447 | O | CYS | 1024 | 30.362 | 21.934 | 23.078 | 1.00 | 12.10 |
| ATOM | 1448 | N | ILE | 1025 | 29.808 | 20.074 | 24.231 | 1.00 | 14.70 |
| ATOM | 1449 | H | ILE | 1025 | 30.108 | 19.364 | 24.830 | 1.00 | 0.00 |
| ATOM | 1450 | CA | ILE | 1025 | 28.453 | 20.001 | 23.621 | 1.00 | 14.85 |
| ATOM | 1451 | CB | ILE | 1025 | 28.395 | 18.968 | 22.421 | 1.00 | 16.88 |
| ATOM | 1452 | CG2 | ILE | 1025 | 29.761 | 18.511 | 21.859 | 1.00 | 19.25 |
| ATOM | 1453 | CG1 | ILE | 1025 | 27.679 | 17.811 | 22.893 | 1.00 | 18.10 |
| ATOM | 1454 | CD1 | ILE | 1025 | 27.340 | 17.009 | 21.679 | 1.00 | 21.23 |
| ATOM | 1455 | C | ILE | 1025 | 27.544 | 19.654 | 24.808 | 1.00 | 11.96 |
| ATOM | 1456 | O | ILE | 1025 | 27.972 | 18.997 | 25.730 | 1.00 | 13.55 |
| ATOM | 1457 | N | HIS | 1026 | 26.373 | 20.227 | 24.935 | 1.00 | 10.63 |
| ATOM | 1458 | H | HIS | 1026 | 26.162 | 20.925 | 24.289 | 1.00 | 0.00 |
| ATOM | 1459 | CA | HIS | 1026 | 25.411 | 20.042 | 26.069 | 1.00 | 9.22 |
| ATOM | 1460 | C | HIS | 1026 | 24.810 | 18.663 | 26.202 | 1.00 | 10.49 |
| ATOM | 1461 | O | HIS | 1026 | 24.815 | 18.051 | 27.254 | 1.00 | 11.75 |
| ATOM | 1462 | CB | HIS | 1026 | 24.280 | 21.126 | 25.916 | 1.00 | 6.98 |
| ATOM | 1463 | CG | HIS | 1026 | 23.425 | 21.211 | 27.090 | 1.00 | 6.84 |
| ATOM | 1464 | ND1 | HIS | 1026 | 22.431 | 20.327 | 27.403 | 1.00 | 8.17 |
| ATOM | 1465 | CE1 | HIS | 1026 | 21.925 | 20.707 | 28.630 | 1.00 | 7.86 |
| ATOM | 1466 | CD2 | HIS | 1026 | 23.542 | 22.150 | 28.125 | 1.00 | 6.70 |
| ATOM | 1467 | NE2 | HIS | 1026 | 22.608 | 21.825 | 29.072 | 1.00 | 9.38 |
| ATOM | 1468 | HE2 | HIS | 1026 | 22.435 | 22.294 | 29.911 | 1.00 | 0.00 |
| ATOM | 1469 | N | ARG | 1027 | 24.378 | 18.108 | 25.081 | 1.00 | 10.74 |
| ATOM | 1470 | H | ARG | 1027 | 24.550 | 18.606 | 24.261 | 1.00 | 0.00 |
| ATOM | 1471 | CA | ARG | 1027 | 23.742 | 16.776 | 24.986 | 1.00 | 12.73 |
| ATOM | 1472 | CB | ARG | 1027 | 24.700 | 15.671 | 25.468 | 1.00 | 12.67 |
| ATOM | 1473 | CG | ARG | 1027 | 25.968 | 15.549 | 24.601 | 1.00 | 9.83 |
| ATOM | 1474 | CD | ARG | 1027 | 26.858 | 14.555 | 25.387 | 1.00 | 10.41 |
| ATOM | 1475 | NE | ARG | 1027 | 28.157 | 14.410 | 24.699 | 1.00 | 15.10 |
| ATOM | 1476 | HE | ARG | 1027 | 28.264 | 13.709 | 24.019 | 1.00 | 0.00 |
| ATOM | 1477 | CZ | ARG | 1027 | 29.182 | 15.226 | 24.985 | 1.00 | 13.83 |
| ATOM | 1478 | NH1 | ARG | 1027 | 30.307 | 15.076 | 24.330 | 1.00 | 13.04 |
| ATOM | 1479 | HH11 | ARG | 1027 | 30.391 | 14.366 | 23.636 | 1.00 | 0.00 |
| ATOM | 1480 | HH12 | ARG | 1027 | 31.086 | 15.667 | 24.525 | 1.00 | 0.00 |
| ATOM | 1481 | NH2 | ARG | 1027 | 29.086 | 16.150 | 25.949 | 1.00 | 10.90 |
| ATOM | 1482 | HH21 | ARG | 1027 | 28.239 | 16.259 | 26.465 | 1.00 | 0.00 |
| ATOM | 1483 | HH22 | ARG | 1027 | 29.868 | 16.729 | 26.151 | 1.00 | 0.00 |
| ATOM | 1484 | C | ARG | 1027 | 22.375 | 16.622 | 25.693 | 1.00 | 13.99 |
| ATOM | 1485 | O | ARG | 1027 | 21.731 | 15.565 | 25.574 | 1.00 | 16.20 |
| ATOM | 1486 | N | ASP | 1028 | 21.843 | 17.677 | 26.341 | 1.00 | 13.69 |
| ATOM | 1487 | H | ASP | 1028 | 22.452 | 18.401 | 26.590 | 1.00 | 0.00 |
| ATOM | 1488 | CA | ASP | 1028 | 20.468 | 17.630 | 26.910 | 1.00 | 12.87 |
| ATOM | 1489 | CB | ASP | 1028 | 20.446 | 17.127 | 28.378 | 1.00 | 12.03 |
| ATOM | 1490 | CG | ASP | 1028 | 19.026 | 16.750 | 28.811 | 1.00 | 11.69 |
| ATOM | 1491 | OD1 | ASP | 1028 | 18.871 | 16.469 | 29.986 | 1.00 | 14.54 |
| ATOM | 1492 | OD2 | ASP | 1028 | 18.045 | 16.750 | 28.055 | 1.00 | 13.13 |
| ATOM | 1493 | C | ASP | 1028 | 19.844 | 19.036 | 26.877 | 1.00 | 12.52 |
| ATOM | 1494 | O | ASP | 1028 | 19.275 | 19.582 | 27.864 | 1.00 | 12.26 |
| ATOM | 1495 | N | LEU | 1029 | 20.013 | 19.698 | 25.713 | 1.00 | 10.66 |
| ATOM | 1496 | H | LEU | 1029 | 20.579 | 19.360 | 25.003 | 1.00 | 0.00 |
| ATOM | 1497 | CA | LEU | 1029 | 19.438 | 21.049 | 25.629 | 1.00 | 10.05 |
| ATOM | 1498 | CB | LEU | 1029 | 20.055 | 21.804 | 24.444 | 1.00 | 9.87 |
| ATOM | 1499 | CG | LEU | 1029 | 19.745 | 23.331 | 24.447 | 1.00 | 11.08 |
| ATOM | 1500 | CD1 | LEU | 1029 | 20.401 | 24.117 | 25.641 | 1.00 | 10.83 |
| ATOM | 1501 | CD2 | LEU | 1029 | 20.355 | 23.900 | 23.150 | 1.00 | 11.31 |
| ATOM | 1502 | C | LEU | 1029 | 17.903 | 21.020 | 25.535 | 1.00 | 10.51 |
| ATOM | 1503 | O | LEU | 1029 | 17.300 | 20.316 | 24.724 | 1.00 | 8.73 |
| ATOM | 1504 | N | ALA | 1030 | 17.265 | 21.842 | 26.370 | 1.00 | 10.79 |
| ATOM | 1505 | H | ALA | 1030 | 17.822 | 22.388 | 26.964 | 1.00 | 0.00 |
| ATOM | 1506 | CA | ALA | 1030 | 15.796 | 21.933 | 26.476 | 1.00 | 10.11 |
| ATOM | 1507 | CB | ALA | 1030 | 15.238 | 20.607 | 27.134 | 1.00 | 6.52 |
| ATOM | 1508 | C | ALA | 1030 | 15.443 | 23.129 | 27.359 | 1.00 | 11.69 |
| ATOM | 1509 | O | ALA | 1030 | 16.298 | 23.600 | 28.125 | 1.00 | 12.45 |
| ATOM | 1510 | N | ALA | 1031 | 14.213 | 23.651 | 27.302 | 1.00 | 10.56 |
| ATOM | 1511 | H | ALA | 1031 | 13.647 | 23.371 | 26.568 | 1.00 | 0.00 |
| ATOM | 1512 | CA | ALA | 1031 | 13.843 | 24.803 | 28.171 | 1.00 | 10.42 |
| ATOM | 1513 | CB | ALA | 1031 | 12.428 | 25.313 | 27.770 | 1.00 | 6.37 |
| ATOM | 1514 | C | ALA | 1031 | 13.910 | 24.473 | 29.688 | 1.00 | 9.09 |
| ATOM | 1515 | O | ALA | 1031 | 14.209 | 25.307 | 30.534 | 1.00 | 6.26 |
| ATOM | 1516 | N | ARG | 1032 | 13.675 | 23.215 | 30.034 | 1.00 | 10.67 |
| ATOM | 1517 | H | ARG | 1032 | 13.291 | 22.626 | 29.355 | 1.00 | 0.00 |
| ATOM | 1518 | CA | ARG | 1032 | 13.795 | 22.686 | 31.422 | 1.00 | 9.19 |
| ATOM | 1519 | CB | ARG | 1032 | 13.526 | 21.183 | 31.448 | 1.00 | 11.09 |
| ATOM | 1520 | CG | ARG | 1032 | 14.574 | 20.281 | 30.757 | 1.00 | 6.49 |
| ATOM | 1521 | CD | ARG | 1032 | 14.614 | 18.710 | 31.028 | 1.00 | 13.42 |
| ATOM | 1522 | NE | ARG | 1032 | 13.960 | 18.263 | 29.846 | 1.00 | 16.78 |
| ATOM | 1523 | HE | ARG | 1032 | 13.001 | 18.479 | 29.881 | 1.00 | 0.00 |
| ATOM | 1524 | CZ | ARG | 1032 | 14.282 | 17.665 | 28.723 | 1.00 | 13.73 |
| ATOM | 1525 | NH1 | ARG | 1032 | 13.187 | 17.637 | 28.035 | 1.00 | 15.98 |
| ATOM | 1526 | HH11 | ARG | 1032 | 12.348 | 18.031 | 28.412 | 1.00 | 0.00 |
| ATOM | 1527 | HH12 | ARG | 1032 | 13.198 | 17.228 | 27.128 | 1.00 | 0.00 |
| ATOM | 1528 | NH2 | ARG | 1032 | 15.375 | 17.139 | 28.213 | 1.00 | 14.13 |
| ATOM | 1529 | HH21 | ARG | 1032 | 16.220 | 17.136 | 28.748 | 1.00 | 0.00 |
| ATOM | 1530 | HH22 | ARG | 1032 | 15.350 | 16.738 | 27.301 | 1.00 | 0.00 |
| ATOM | 1531 | C | ARG | 1032 | 15.208 | 22.914 | 31.922 | 1.00 | 11.22 |
| ATOM | 1532 | O | ARG | 1032 | 15.509 | 22.945 | 33.122 | 1.00 | 11.21 |
| ATOM | 1533 | N | ASN | 1033 | 16.168 | 22.894 | 30.988 | 1.00 | 11.36 |
| ATOM | 1534 | H | ASN | 1033 | 15.926 | 22.729 | 30.056 | 1.00 | 0.00 |
| ATOM | 1535 | CA | ASN | 1033 | 17.595 | 23.168 | 31.340 | 1.00 | 11.75 |
| ATOM | 1536 | CB | ASN | 1033 | 18.525 | 22.199 | 30.567 | 1.00 | 10.47 |
| ATOM | 1537 | CG | ASN | 1033 | 18.570 | 20.885 | 31.336 | 1.00 | 9.64 |
| ATOM | 1538 | OD1 | ASN | 1033 | 18.513 | 20.835 | 32.561 | 1.00 | 8.65 |
| ATOM | 1539 | ND2 | ASN | 1033 | 18.687 | 19.759 | 30.673 | 1.00 | 10.81 |
| ATOM | 1540 | HD21 | ASN | 1033 | 18.759 | 19.767 | 29.697 | 1.00 | 0.00 |
| ATOM | 1541 | HD22 | ASN | 1033 | 18.709 | 18.900 | 31.132 | 1.00 | 0.00 |
| ATOM | 1542 | C | ASN | 1033 | 18.040 | 24.618 | 31.113 | 1.00 | 9.05 |
| ATOM | 1543 | O | ASN | 1033 | 19.228 | 24.946 | 30.995 | 1.00 | 10.84 |
| ATOM | 1544 | N | ILE | 1034 | 17.078 | 25.514 | 31.030 | 1.00 | 9.16 |
| ATOM | 1545 | H | ILE | 1034 | 16.149 | 25.236 | 31.141 | 1.00 | 0.00 |
| ATOM | 1546 | CA | ILE | 1034 | 17.403 | 26.944 | 30.862 | 1.00 | 9.34 |
| ATOM | 1547 | CB | ILE | 1034 | 16.752 | 27.614 | 29.606 | 1.00 | 7.21 |
| ATOM | 1548 | CG2 | ILE | 1034 | 17.164 | 29.153 | 29.643 | 1.00 | 9.24 |
| ATOM | 1549 | CG1 | ILE | 1034 | 17.184 | 26.971 | 28.281 | 1.00 | 5.02 |
| ATOM | 1550 | CD1 | ILE | 1034 | 18.689 | 26.900 | 28.009 | 1.00 | 2.00 |
| ATOM | 1551 | C | ILE | 1034 | 16.873 | 27.660 | 32.090 | 1.00 | 9.90 |
| ATOM | 1552 | O | ILE | 1034 | 15.774 | 27.366 | 32.557 | 1.00 | 10.84 |
| ATOM | 1553 | N | LEU | 1035 | 17.714 | 28.438 | 32.735 | 1.00 | 9.12 |
| ATOM | 1554 | H | LEU | 1035 | 18.641 | 28.401 | 32.463 | 1.00 | 0.00 |
| ATOM | 1555 | CA | LEU | 1035 | 17.319 | 29.207 | 33.901 | 1.00 | 10.48 |
| ATOM | 1556 | CB | LEU | 1035 | 18.405 | 29.071 | 34.986 | 1.00 | 10.95 |
| ATOM | 1557 | CG | LEU | 1035 | 18.601 | 27.645 | 35.532 | 1.00 | 6.61 |
| ATOM | 1558 | CD1 | LEU | 1035 | 19.771 | 27.734 | 36.442 | 1.00 | 7.72 |
| ATOM | 1559 | CD2 | LEU | 1035 | 17.393 | 27.063 | 36.292 | 1.00 | 11.29 |
| ATOM | 1560 | C | LEU | 1035 | 17.045 | 30.706 | 33.668 | 1.00 | 12.33 |
| ATOM | 1561 | O | LEU | 1035 | 17.762 | 31.392 | 32.914 | 1.00 | 12.66 |
| ATOM | 1562 | N | LEU | 1036 | 15.984 | 31.244 | 34.273 | 1.00 | 11.39 |
| ATOM | 1563 | H | LEU | 1036 | 15.359 | 30.661 | 34.748 | 1.00 | 0.00 |
| ATOM | 1564 | CA | LEU | 1036 | 15.718 | 32.691 | 34.166 | 1.00 | 13.22 |
| ATOM | 1565 | CB | LEU | 1036 | 14.188 | 32.960 | 33.846 | 1.00 | 9.17 |
| ATOM | 1566 | CG | LEU | 1036 | 13.851 | 34.460 | 33.847 | 1.00 | 7.71 |
| ATOM | 1567 | CD1 | LEU | 1036 | 14.548 | 35.257 | 32.705 | 1.00 | 3.52 |
| ATOM | 1568 | CD2 | LEU | 1036 | 12.365 | 34.565 | 33.676 | 1.00 | 9.99 |
| ATOM | 1569 | C | LEU | 1036 | 16.141 | 33.431 | 35.472 | 1.00 | 14.02 |
| ATOM | 1570 | O | LEU | 1036 | 15.777 | 33.086 | 36.585 | 1.00 | 12.00 |
| ATOM | 1571 | N | SER | 1037 | 17.061 | 34.377 | 35.311 | 1.00 | 17.18 |
| ATOM | 1572 | H | SER | 1037 | 17.344 | 34.481 | 34.384 | 1.00 | 0.00 |
| ATOM | 1573 | CA | SER | 1037 | 17.666 | 35.267 | 36.330 | 1.00 | 18.81 |
| ATOM | 1574 | CB | SER | 1037 | 19.155 | 35.372 | 36.002 | 1.00 | 19.50 |
| ATOM | 1575 | OG | SER | 1037 | 19.864 | 35.418 | 37.222 | 1.00 | 22.75 |
| ATOM | 1576 | HG | SER | 1037 | 20.693 | 35.877 | 37.013 | 1.00 | 0.00 |
| ATOM | 1577 | C | SER | 1037 | 16.996 | 36.637 | 36.305 | 1.00 | 20.71 |
| ATOM | 1578 | O | SER | 1037 | 15.974 | 36.840 | 35.621 | 1.00 | 18.30 |
| ATOM | 1579 | N | GLU | 1038 | 17.433 | 37.645 | 37.051 | 1.00 | 24.83 |
| ATOM | 1580 | H | GLU | 1038 | 18.195 | 37.490 | 37.636 | 1.00 | 0.00 |
| ATOM | 1581 | CA | GLU | 1038 | 16.678 | 38.929 | 36.897 | 1.00 | 29.35 |
| ATOM | 1582 | CB | GLU | 1038 | 16.789 | 40.018 | 37.980 | 1.00 | 36.02 |
| ATOM | 1583 | CG | GLU | 1038 | 17.462 | 39.622 | 39.274 | 1.00 | 45.61 |
| ATOM | 1584 | CD | GLU | 1038 | 18.907 | 39.258 | 39.019 | 1.00 | 55.80 |
| ATOM | 1585 | OE1 | GLU | 1038 | 19.456 | 38.504 | 39.853 | 1.00 | 60.40 |
| ATOM | 1586 | OE2 | GLU | 1038 | 19.497 | 39.799 | 38.062 | 1.00 | 60.58 |
| ATOM | 1587 | C | GLU | 1038 | 17.205 | 39.656 | 35.708 | 1.00 | 28.01 |
| ATOM | 1588 | O | GLU | 1038 | 18.102 | 39.163 | 35.025 | 1.00 | 25.32 |
| ATOM | 1589 | N | LYS | 1039 | 16.645 | 40.831 | 35.413 | 1.00 | 27.99 |
| ATOM | 1590 | H | LYS | 1039 | 15.890 | 41.133 | 35.953 | 1.00 | 0.00 |
| ATOM | 1591 | CA | LYS | 1039 | 17.059 | 41.651 | 34.241 | 1.00 | 27.75 |
| ATOM | 1592 | CB | LYS | 1039 | 18.522 | 42.171 | 34.457 | 1.00 | 29.69 |
| ATOM | 1593 | CG | LYS | 1039 | 18.775 | 42.884 | 35.814 | 1.00 | 36.47 |
| ATOM | 1594 | CD | LYS | 1039 | 17.989 | 44.225 | 36.092 | 1.00 | 43.35 |
| ATOM | 1595 | CE | LYS | 1039 | 16.412 | 44.396 | 35.910 | 1.00 | 48.13 |
| ATOM | 1596 | NZ | LYS | 1039 | 16.046 | 44.712 | 34.492 | 1.00 | 52.56 |
| ATOM | 1597 | HZ1 | LYS | 1039 | 16.473 | 44.019 | 33.838 | 1.00 | 0.00 |
| ATOM | 1598 | HZ2 | LYS | 1039 | 16.413 | 45.657 | 34.250 | 1.00 | 0.00 |
| ATOM | 1599 | HZ3 | LYS | 1039 | 15.017 | 44.707 | 34.360 | 1.00 | 0.00 |
| ATOM | 1600 | C | LYS | 1039 | 16.950 | 40.886 | 32.938 | 1.00 | 24.36 |
| ATOM | 1601 | O | LYS | 1039 | 17.691 | 41.104 | 31.982 | 1.00 | 25.46 |
| ATOM | 1602 | N | ASN | 1040 | 15.962 | 40.005 | 32.887 | 1.00 | 21.96 |
| ATOM | 1603 | H | ASN | 1040 | 15.392 | 39.897 | 33.674 | 1.00 | 0.00 |
| ATOM | 1604 | CA | ASN | 1040 | 15.710 | 39.122 | 31.706 | 1.00 | 21.19 |
| ATOM | 1605 | CB | ASN | 1040 | 15.119 | 39.961 | 30.508 | 1.00 | 20.45 |
| ATOM | 1606 | CG | ASN | 1040 | 13.714 | 40.284 | 30.979 | 1.00 | 22.14 |
| ATOM | 1607 | OD1 | ASN | 1040 | 12.840 | 39.448 | 31.239 | 1.00 | 23.40 |
| ATOM | 1608 | ND2 | ASN | 1040 | 13.437 | 41.532 | 31.224 | 1.00 | 25.07 |
| ATOM | 1609 | HD21 | ASN | 1040 | 14.084 | 42.245 | 31.100 | 1.00 | 0.00 |
| ATOM | 1610 | HD22 | ASN | 1040 | 12.529 | 41.731 | 31.525 | 1.00 | 0.00 |
| ATOM | 1611 | C | ASN | 1040 | 16.894 | 38.273 | 31.213 | 1.00 | 17.74 |
| ATOM | 1612 | O | ASN | 1040 | 16.882 | 37.745 | 30.096 | 1.00 | 19.56 |
| ATOM | 1613 | N | VAL | 1041 | 17.906 | 38.081 | 32.064 | 1.00 | 15.84 |
| ATOM | 1614 | H | VAL | 1041 | 17.869 | 38.522 | 32.939 | 1.00 | 0.00 |
| ATOM | 1615 | CA | VAL | 1041 | 19.059 | 37.228 | 31.727 | 1.00 | 13.19 |
| ATOM | 1616 | CB | VAL | 1041 | 20.286 | 37.563 | 32.576 | 1.00 | 13.28 |
| ATOM | 1617 | CG1 | VAL | 1041 | 21.336 | 36.499 | 32.315 | 1.00 | 9.61 |
| ATOM | 1618 | CG2 | VAL | 1041 | 20.768 | 39.031 | 32.270 | 1.00 | 13.16 |
| ATOM | 1619 | C | VAL | 1041 | 18.721 | 35.774 | 31.940 | 1.00 | 13.16 |
| ATOM | 1620 | O | VAL | 1041 | 18.232 | 35.331 | 32.957 | 1.00 | 12.56 |
| ATOM | 1621 | N | VAL | 1042 | 18.904 | 35.013 | 30.897 | 1.00 | 15.03 |
| ATOM | 1622 | H | VAL | 1042 | 19.271 | 35.463 | 30.101 | 1.00 | 0.00 |
| ATOM | 1623 | CA | VAL | 1042 | 18.649 | 33.569 | 30.817 | 1.00 | 14.96 |
| ATOM | 1624 | CB | VAL | 1042 | 17.698 | 33.509 | 29.532 | 1.00 | 18.56 |
| ATOM | 1625 | CG1 | VAL | 1042 | 18.365 | 32.791 | 28.344 | 1.00 | 15.85 |
| ATOM | 1626 | CG2 | VAL | 1042 | 16.329 | 33.036 | 30.017 | 1.00 | 14.58 |
| ATOM | 1627 | C | VAL | 1042 | 20.015 | 32.850 | 30.774 | 1.00 | 15.57 |
| ATOM | 1628 | O | VAL | 1042 | 20.947 | 33.312 | 30.085 | 1.00 | 14.75 |
| ATOM | 1629 | N | LYS | 1043 | 20.145 | 31.764 | 31.562 | 1.00 | 12.56 |
| ATOM | 1630 | H | LYS | 1043 | 19.370 | 31.524 | 32.101 | 1.00 | 0.00 |
| ATOM | 1631 | CA | LYS | 1043 | 21.348 | 30.947 | 31.657 | 1.00 | 9.54 |
| ATOM | 1632 | CB | LYS | 1043 | 21.942 | 31.062 | 33.084 | 1.00 | 9.57 |
| ATOM | 1633 | CG | LYS | 1043 | 22.641 | 32.436 | 33.176 | 1.00 | 11.90 |
| ATOM | 1634 | CD | LYS | 1043 | 22.870 | 32.850 | 34.631 | 1.00 | 13.24 |
| ATOM | 1635 | CE | LYS | 1043 | 23.518 | 34.254 | 34.779 | 1.00 | 14.88 |
| ATOM | 1636 | NZ | LYS | 1043 | 24.927 | 34.112 | 34.375 | 1.00 | 18.33 |
| ATOM | 1637 | HZ1 | LYS | 1043 | 24.962 | 33.603 | 33.471 | 1.00 | 0.00 |
| ATOM | 1638 | HZ2 | LYS | 1043 | 25.472 | 33.588 | 35.079 | 1.00 | 0.00 |
| ATOM | 1639 | HZ3 | LYS | 1043 | 25.358 | 35.048 | 34.245 | 1.00 | 0.00 |
| ATOM | 1640 | C | LYS | 1043 | 21.165 | 29.466 | 31.344 | 1.00 | 10.95 |
| ATOM | 1641 | O | LYS | 1043 | 20.255 | 28.758 | 31.775 | 1.00 | 11.40 |
| ATOM | 1642 | N | ILE | 1044 | 22.033 | 28.930 | 30.519 | 1.00 | 11.34 |
| ATOM | 1643 | H | ILE | 1044 | 22.662 | 29.532 | 30.064 | 1.00 | 0.00 |
| ATOM | 1644 | CA | ILE | 1044 | 21.997 | 27.509 | 30.213 | 1.00 | 10.08 |
| ATOM | 1645 | CB | ILE | 1044 | 22.864 | 27.220 | 28.925 | 1.00 | 11.69 |
| ATOM | 1646 | CG2 | ILE | 1044 | 22.933 | 25.666 | 28.804 | 1.00 | 10.94 |
| ATOM | 1647 | CG1 | ILE | 1044 | 22.334 | 27.969 | 27.651 | 1.00 | 7.49 |
| ATOM | 1648 | CD1 | ILE | 1044 | 23.351 | 27.794 | 26.585 | 1.00 | 4.06 |
| ATOM | 1649 | C | ILE | 1044 | 22.582 | 26.726 | 31.408 | 1.00 | 11.61 |
| ATOM | 1650 | O | ILE | 1044 | 23.648 | 27.073 | 31.976 | 1.00 | 9.99 |
| ATOM | 1651 | N | CYS | 1045 | 21.892 | 25.664 | 31.829 | 1.00 | 10.61 |
| ATOM | 1652 | H | CYS | 1045 | 21.055 | 25.439 | 31.375 | 1.00 | 0.00 |
| ATOM | 1653 | CA | CYS | 1045 | 22.406 | 24.800 | 32.958 | 1.00 | 9.79 |
| ATOM | 1654 | CB | CYS | 1045 | 21.660 | 25.076 | 34.350 | 1.00 | 8.31 |
| ATOM | 1655 | SG | CYS | 1045 | 19.915 | 24.438 | 34.310 | 1.00 | 10.37 |
| ATOM | 1656 | C | CYS | 1045 | 22.167 | 23.316 | 32.598 | 1.00 | 9.48 |
| ATOM | 1657 | O | CYS | 1045 | 21.717 | 22.927 | 31.511 | 1.00 | 8.76 |
| ATOM | 1658 | N | ASP | 1046 | 22.595 | 22.415 | 33.436 | 1.00 | 7.87 |
| ATOM | 1659 | H | ASP | 1046 | 23.148 | 22.686 | 34.200 | 1.00 | 0.00 |
| ATOM | 1660 | CA | ASP | 1046 | 22.328 | 21.023 | 33.162 | 1.00 | 7.87 |
| ATOM | 1661 | CB | ASP | 1046 | 23.585 | 20.291 | 32.617 | 1.00 | 9.37 |
| ATOM | 1662 | CG | ASP | 1046 | 23.262 | 18.868 | 32.170 | 1.00 | 11.43 |
| ATOM | 1663 | OD1 | ASP | 1046 | 22.226 | 18.314 | 32.468 | 1.00 | 14.63 |
| ATOM | 1664 | OD2 | ASP | 1046 | 24.038 | 18.283 | 31.440 | 1.00 | 17.23 |
| ATOM | 1665 | C | ASP | 1046 | 21.958 | 20.443 | 34.526 | 1.00 | 9.02 |
| ATOM | 1666 | O | ASP | 1046 | 22.910 | 20.306 | 35.294 | 1.00 | 8.55 |
| ATOM | 1667 | N | PHE | 1047 | 20.654 | 20.175 | 34.866 | 1.00 | 7.75 |
| ATOM | 1668 | H | PHE | 1047 | 19.946 | 20.573 | 34.318 | 1.00 | 0.00 |
| ATOM | 1669 | CA | PHE | 1047 | 20.293 | 19.578 | 36.165 | 1.00 | 8.31 |
| ATOM | 1670 | CB | PHE | 1047 | 18.768 | 19.294 | 36.318 | 1.00 | 8.07 |
| ATOM | 1671 | CG | PHE | 1047 | 18.080 | 20.506 | 36.782 | 1.00 | 7.57 |
| ATOM | 1672 | CD1 | PHE | 1047 | 17.852 | 21.569 | 35.913 | 1.00 | 8.44 |
| ATOM | 1673 | CD2 | PHE | 1047 | 17.615 | 20.573 | 38.110 | 1.00 | 10.21 |
| ATOM | 1674 | CE1 | PHE | 1047 | 17.145 | 22.707 | 36.349 | 1.00 | 9.62 |
| ATOM | 1675 | CE2 | PHE | 1047 | 16.910 | 21.689 | 38.545 | 1.00 | 10.56 |
| ATOM | 1676 | CZ | PHE | 1047 | 16.668 | 22.762 | 37.673 | 1.00 | 12.10 |
| ATOM | 1677 | C | PHE | 1047 | 21.009 | 18.273 | 36.378 | 1.00 | 9.17 |
| ATOM | 1678 | O | PHE | 1047 | 21.472 | 17.919 | 37.477 | 1.00 | 9.77 |
| ATOM | 1679 | N | GLY | 1048 | 21.121 | 17.499 | 35.320 | 1.00 | 10.49 |
| ATOM | 1680 | H | GLY | 1048 | 20.700 | 17.793 | 34.493 | 1.00 | 0.00 |
| ATOM | 1681 | CA | GLY | 1048 | 21.839 | 16.221 | 35.365 | 1.00 | 9.67 |
| ATOM | 1682 | C | GLY | 1048 | 21.333 | 15.323 | 36.487 | 1.00 | 12.55 |
| ATOM | 1683 | O | GLY | 1048 | 20.136 | 15.036 | 36.597 | 1.00 | 14.91 |
| ATOM | 1684 | N | LEU | 1049 | 22.224 | 14.942 | 37.385 | 1.00 | 14.19 |
| ATOM | 1685 | H | LEU | 1049 | 23.112 | 15.357 | 37.324 | 1.00 | 0.00 |
| ATOM | 1686 | CA | LEU | 1049 | 21.836 | 14.046 | 38.481 | 1.00 | 15.61 |
| ATOM | 1687 | CB | LEU | 1049 | 23.079 | 13.694 | 39.303 | 1.00 | 19.24 |
| ATOM | 1688 | CG | LEU | 1049 | 23.981 | 12.438 | 39.035 | 1.00 | 22.75 |
| ATOM | 1689 | CD1 | LEU | 1049 | 23.880 | 11.802 | 37.633 | 1.00 | 22.87 |
| ATOM | 1690 | CD2 | LEU | 1049 | 25.384 | 12.928 | 39.431 | 1.00 | 22.76 |
| ATOM | 1691 | C | LEU | 1049 | 20.777 | 14.690 | 39.342 | 1.00 | 14.81 |
| ATOM | 1692 | O | LEU | 1049 | 20.010 | 13.960 | 39.980 | 1.00 | 15.38 |
| ATOM | 1693 | N | ALA | 1050 | 20.693 | 16.041 | 39.378 | 1.00 | 11.88 |
| ATOM | 1694 | H | ALA | 1050 | 21.360 | 16.569 | 38.888 | 1.00 | 0.00 |
| ATOM | 1695 | CA | ALA | 1050 | 19.647 | 16.688 | 40.210 | 1.00 | 11.99 |
| ATOM | 1696 | CB | ALA | 1050 | 19.893 | 18.184 | 40.463 | 1.00 | 7.80 |
| ATOM | 1697 | C | ALA | 1050 | 18.277 | 16.583 | 39.553 | 1.00 | 12.29 |
| ATOM | 1698 | O | ALA | 1050 | 17.314 | 17.249 | 39.934 | 1.00 | 11.89 |
| ATOM | 1699 | N | ARG | 1051 | 18.156 | 15.783 | 38.500 | 1.00 | 14.11 |
| ATOM | 1700 | H | ARG | 1051 | 18.922 | 15.289 | 38.141 | 1.00 | 0.00 |
| ATOM | 1701 | CA | ARG | 1051 | 16.850 | 15.630 | 37.878 | 1.00 | 17.49 |
| ATOM | 1702 | CB | ARG | 1051 | 16.850 | 16.068 | 36.409 | 1.00 | 16.90 |
| ATOM | 1703 | CG | ARG | 1051 | 15.399 | 15.930 | 35.894 | 1.00 | 14.56 |
| ATOM | 1704 | CD | ARG | 1051 | 15.273 | 16.340 | 34.472 | 1.00 | 17.05 |
| ATOM | 1705 | NE | ARG | 1051 | 15.182 | 17.813 | 34.459 | 1.00 | 15.27 |
| ATOM | 1706 | HE | ARG | 1051 | 14.384 | 18.221 | 34.859 | 1.00 | 0.00 |
| ATOM | 1707 | CZ | ARG | 1051 | 16.126 | 18.616 | 33.927 | 1.00 | 14.54 |
| ATOM | 1708 | NH1 | ARG | 1051 | 15.909 | 19.926 | 33.978 | 1.00 | 12.19 |
| ATOM | 1709 | HH11 | ARG | 1051 | 15.067 | 20.272 | 34.398 | 1.00 | 0.00 |
| ATOM | 1710 | HH12 | ARG | 1051 | 16.583 | 20.561 | 33.602 | 1.00 | 0.00 |
| ATOM | 1711 | NH2 | ARG | 1051 | 17.273 | 18.147 | 33.374 | 1.00 | 15.98 |
| ATOM | 1712 | HH21 | ARG | 1051 | 17.455 | 17.165 | 33.356 | 1.00 | 0.00 |
| ATOM | 1713 | HH22 | ARG | 1051 | 17.923 | 18.795 | 32.977 | 1.00 | 0.00 |
| ATOM | 1714 | C | ARG | 1051 | 16.448 | 14.174 | 37.936 | 1.00 | 20.25 |
| ATOM | 1715 | O | ARG | 1051 | 17.279 | 13.276 | 37.756 | 1.00 | 18.31 |
| ATOM | 1716 | N | ASP | 1052 | 15.214 | 13.937 | 38.348 | 1.00 | 23.67 |
| ATOM | 1717 | H | ASP | 1052 | 14.683 | 14.685 | 38.682 | 1.00 | 0.00 |
| ATOM | 1718 | CA | ASP | 1052 | 14.720 | 12.536 | 38.408 | 1.00 | 28.87 |
| ATOM | 1719 | CB | ASP | 1052 | 13.498 | 12.408 | 39.382 | 1.00 | 33.96 |
| ATOM | 1720 | CG | ASP | 1052 | 13.253 | 10.968 | 39.921 | 1.00 | 39.68 |
| ATOM | 1721 | OD1 | ASP | 1052 | 13.767 | 9.963 | 39.374 | 1.00 | 40.34 |
| ATOM | 1722 | OD2 | ASP | 1052 | 12.511 | 10.899 | 40.923 | 1.00 | 43.88 |
| ATOM | 1723 | C | ASP | 1052 | 14.298 | 12.045 | 37.004 | 1.00 | 27.42 |
| ATOM | 1724 | O | ASP | 1052 | 13.113 | 12.014 | 36.647 | 1.00 | 26.06 |
| ATOM | 1725 | N | ILE | 1053 | 15.258 | 11.631 | 36.194 | 1.00 | 26.62 |
| ATOM | 1726 | H | ILE | 1053 | 16.188 | 11.719 | 36.489 | 1.00 | 0.00 |
| ATOM | 1727 | CA | ILE | 1053 | 14.918 | 11.187 | 34.830 | 1.00 | 28.09 |
| ATOM | 1728 | CB | ILE | 1053 | 16.175 | 10.884 | 33.934 | 1.00 | 27.47 |
| ATOM | 1729 | CG2 | ILE | 1053 | 16.889 | 12.245 | 33.660 | 1.00 | 23.05 |
| ATOM | 1730 | CG1 | ILE | 1053 | 17.035 | 9.749 | 34.552 | 1.00 | 23.01 |
| ATOM | 1731 | CD1 | ILE | 1053 | 17.858 | 9.197 | 33.398 | 1.00 | 23.35 |
| ATOM | 1732 | C | ILE | 1053 | 14.055 | 9.959 | 34.788 | 1.00 | 30.28 |
| ATOM | 1733 | O | ILE | 1053 | 13.642 | 9.582 | 33.695 | 1.00 | 30.40 |
| ATOM | 1734 | N | TYR | 1054 | 13.922 | 9.234 | 35.915 | 1.00 | 32.32 |
| ATOM | 1735 | H | TYR | 1054 | 14.483 | 9.440 | 36.691 | 1.00 | 0.00 |
| ATOM | 1736 | CA | TYR | 1054 | 13.018 | 8.056 | 35.880 | 1.00 | 33.97 |
| ATOM | 1737 | CB | TYR | 1054 | 13.479 | 6.971 | 36.888 | 1.00 | 38.47 |
| ATOM | 1738 | CG | TYR | 1054 | 14.775 | 6.491 | 36.316 | 1.00 | 45.06 |
| ATOM | 1739 | CD1 | TYR | 1054 | 15.991 | 6.664 | 37.033 | 1.00 | 47.12 |
| ATOM | 1740 | CE1 | TYR | 1054 | 17.232 | 6.322 | 36.454 | 1.00 | 47.98 |
| ATOM | 1741 | CD2 | TYR | 1054 | 14.813 | 5.950 | 35.010 | 1.00 | 47.93 |
| ATOM | 1742 | CE2 | TYR | 1054 | 16.057 | 5.610 | 34.433 | 1.00 | 49.46 |
| ATOM | 1743 | CZ | TYR | 1054 | 17.268 | 5.802 | 35.143 | 1.00 | 49.10 |
| ATOM | 1744 | OH | TYR | 1054 | 18.493 | 5.603 | 34.492 | 1.00 | 51.35 |
| ATOM | 1745 | HH | TYR | 1054 | 18.341 | 5.072 | 33.705 | 1.00 | 0.00 |
| ATOM | 1746 | C | TYR | 1054 | 11.589 | 8.461 | 36.128 | 1.00 | 32.04 |
| ATOM | 1747 | O | TYR | 1054 | 10.669 | 7.800 | 35.663 | 1.00 | 33.39 |
| ATOM | 1748 | N | LYS | 1055 | 11.355 | 9.520 | 36.888 | 1.00 | 29.69 |
| ATOM | 1749 | H | LYS | 1055 | 12.107 | 10.043 | 37.225 | 1.00 | 0.00 |
| ATOM | 1750 | CA | LYS | 1055 | 9.964 | 9.949 | 37.088 | 1.00 | 28.93 |
| ATOM | 1751 | CB | LYS | 1055 | 9.790 | 10.467 | 38.528 | 1.00 | 27.00 |
| ATOM | 1752 | C | LYS | 1055 | 9.545 | 11.066 | 36.059 | 1.00 | 29.21 |
| ATOM | 1753 | O | LYS | 1055 | 8.366 | 11.364 | 35.786 | 1.00 | 28.05 |
| ATOM | 1754 | N | ASP | 1056 | 10.537 | 11.740 | 35.453 | 1.00 | 27.94 |
| ATOM | 1755 | H | ASP | 1056 | 11.461 | 11.468 | 35.610 | 1.00 | 0.00 |
| ATOM | 1756 | CA | ASP | 1056 | 10.242 | 12.822 | 34.508 | 1.00 | 24.87 |
| ATOM | 1757 | CB | ASP | 1056 | 11.519 | 13.674 | 34.374 | 1.00 | 25.78 |
| ATOM | 1758 | CG | ASP | 1056 | 11.298 | 15.021 | 33.685 | 1.00 | 25.28 |
| ATOM | 1759 | OD1 | ASP | 1056 | 12.067 | 15.910 | 33.964 | 1.00 | 30.36 |
| ATOM | 1760 | OD2 | ASP | 1056 | 10.421 | 15.225 | 32.856 | 1.00 | 26.40 |
| ATOM | 1761 | C | ASP | 1056 | 9.741 | 12.361 | 33.141 | 1.00 | 23.43 |
| ATOM | 1762 | O | ASP | 1056 | 10.502 | 11.747 | 32.401 | 1.00 | 22.01 |
| ATOM | 1763 | N | PRO | 1057 | 8.489 | 12.739 | 32.729 | 1.00 | 21.95 |
| ATOM | 1764 | CD | PRO | 1057 | 7.567 | 13.668 | 33.417 | 1.00 | 18.33 |
| ATOM | 1765 | CA | PRO | 1057 | 7.909 | 12.286 | 31.441 | 1.00 | 20.12 |
| ATOM | 1766 | CB | PRO | 1057 | 6.511 | 12.885 | 31.507 | 1.00 | 18.14 |
| ATOM | 1767 | CG | PRO | 1057 | 6.694 | 14.181 | 32.297 | 1.00 | 17.76 |
| ATOM | 1768 | C | PRO | 1057 | 8.734 | 12.655 | 30.168 | 1.00 | 20.26 |
| ATOM | 1769 | O | PRO | 1057 | 8.606 | 12.051 | 29.098 | 1.00 | 21.56 |
| ATOM | 1770 | N | ASP | 1058 | 9.587 | 13.684 | 30.210 | 1.00 | 18.09 |
| ATOM | 1771 | H | ASP | 1058 | 9.613 | 14.207 | 31.033 | 1.00 | 0.00 |
| ATOM | 1772 | CA | ASP | 1058 | 10.434 | 14.056 | 29.046 | 1.00 | 15.51 |
| ATOM | 1773 | CB | ASP | 1058 | 11.107 | 15.361 | 29.351 | 1.00 | 14.15 |
| ATOM | 1774 | CG | ASP | 1058 | 10.173 | 16.493 | 29.574 | 1.00 | 13.39 |
| ATOM | 1775 | OD1 | ASP | 1058 | 10.650 | 17.488 | 30.050 | 1.00 | 15.70 |
| ATOM | 1776 | OD2 | ASP | 1058 | 9.012 | 16.472 | 29.220 | 1.00 | 17.87 |
| ATOM | 1777 | C | ASP | 1058 | 11.539 | 13.010 | 28.721 | 1.00 | 16.99 |
| ATOM | 1778 | O | ASP | 1058 | 12.246 | 13.115 | 27.715 | 1.00 | 16.49 |
| ATOM | 1779 | N | TYR | 1059 | 11.873 | 12.101 | 29.657 | 1.00 | 16.57 |
| ATOM | 1780 | H | TYR | 1059 | 11.359 | 12.038 | 30.495 | 1.00 | 0.00 |
| ATOM | 1781 | CA | TYR | 1059 | 12.914 | 11.092 | 29.385 | 1.00 | 17.68 |
| ATOM | 1782 | CB | TYR | 1059 | 13.955 | 11.014 | 30.528 | 1.00 | 15.96 |
| ATOM | 1783 | CG | TYR | 1059 | 14.552 | 12.341 | 30.674 | 1.00 | 14.86 |
| ATOM | 1784 | CD1 | TYR | 1059 | 13.904 | 13.360 | 31.381 | 1.00 | 13.40 |
| ATOM | 1785 | CE1 | TYR | 1059 | 14.485 | 14.623 | 31.460 | 1.00 | 13.99 |
| ATOM | 1786 | CD2 | TYR | 1059 | 15.777 | 12.558 | 30.050 | 1.00 | 15.00 |
| ATOM | 1787 | CE2 | TYR | 1059 | 16.352 | 13.824 | 30.134 | 1.00 | 14.01 |
| ATOM | 1788 | CZ | TYR | 1059 | 15.711 | 14.854 | 30.829 | 1.00 | 15.08 |
| ATOM | 1789 | OH | TYR | 1059 | 16.299 | 16.110 | 30.867 | 1.00 | 15.86 |
| ATOM | 1790 | HH | TYR | 1059 | 16.036 | 16.560 | 31.673 | 1.00 | 0.00 |
| ATOM | 1791 | C | TYR | 1059 | 12.277 | 9.734 | 29.205 | 1.00 | 21.10 |
| ATOM | 1792 | O | TYR | 1059 | 11.665 | 9.135 | 30.092 | 1.00 | 24.79 |
| ATOM | 1793 | N | VAL | 1060 | 12.325 | 9.283 | 27.979 | 1.00 | 22.24 |
| ATOM | 1794 | H | VAL | 1060 | 12.669 | 9.898 | 27.312 | 1.00 | 0.00 |
| ATOM | 1795 | CA | VAL | 1060 | 11.742 | 8.029 | 27.564 | 1.00 | 23.11 |
| ATOM | 1796 | CB | VAL | 1060 | 11.071 | 8.388 | 26.254 | 1.00 | 22.46 |
| ATOM | 1797 | CG1 | VAL | 1060 | 10.491 | 7.145 | 25.649 | 1.00 | 25.32 |
| ATOM | 1798 | CG2 | VAL | 1060 | 9.935 | 9.406 | 26.489 | 1.00 | 22.78 |
| ATOM | 1799 | C | VAL | 1060 | 12.768 | 6.937 | 27.484 | 1.00 | 25.40 |
| ATOM | 1800 | O | VAL | 1060 | 13.882 | 7.084 | 26.991 | 1.00 | 24.61 |
| ATOM | 1801 | N | ARG | 1061 | 12.400 | 5.782 | 28.015 | 1.00 | 30.80 |
| ATOM | 1802 | H | ARG | 1061 | 11.497 | 5.733 | 28.380 | 1.00 | 0.00 |
| ATOM | 1803 | CA | ARG | 1061 | 13.282 | 4.564 | 28.023 | 1.00 | 34.53 |
| ATOM | 1804 | CB | ARG | 1061 | 12.652 | 3.354 | 28.805 | 1.00 | 34.35 |
| ATOM | 1805 | C | ARG | 1061 | 13.615 | 4.018 | 26.613 | 1.00 | 36.51 |
| ATOM | 1806 | O | ARG | 1061 | 12.749 | 3.748 | 25.763 | 1.00 | 36.42 |
| ATOM | 1807 | N | LYS | 1062 | 14.911 | 3.879 | 26.403 | 1.00 | 38.48 |
| ATOM | 1808 | H | LYS | 1062 | 15.496 | 4.242 | 27.088 | 1.00 | 0.00 |
| ATOM | 1809 | CA | LYS | 1062 | 15.505 | 3.366 | 25.176 | 1.00 | 41.27 |
| ATOM | 1810 | CB | LYS | 1062 | 15.904 | 4.549 | 24.275 | 1.00 | 41.79 |
| ATOM | 1811 | C | LYS | 1062 | 16.716 | 2.574 | 25.677 | 1.00 | 43.77 |
| ATOM | 1812 | O | LYS | 1062 | 17.708 | 3.063 | 26.242 | 1.00 | 43.79 |
| ATOM | 1813 | N | GLY | 1063 | 16.581 | 1.250 | 25.513 | 1.00 | 46.39 |
| ATOM | 1814 | H | GLY | 1063 | 15.770 | 0.942 | 25.064 | 1.00 | 0.00 |
| ATOM | 1815 | CA | GLY | 1063 | 17.582 | 0.279 | 26.004 | 1.00 | 46.40 |
| ATOM | 1816 | C | GLY | 1063 | 17.581 | 0.397 | 27.545 | 1.00 | 47.48 |
| ATOM | 1817 | O | GLY | 1063 | 16.550 | 0.314 | 28.223 | 1.00 | 47.46 |
| ATOM | 1818 | N | ASP | 1064 | 18.748 | 0.532 | 28.142 | 1.00 | 49.07 |
| ATOM | 1819 | H | ASP | 1064 | 19.553 | 0.570 | 27.589 | 1.00 | 0.00 |
| ATOM | 1820 | CA | ASP | 1064 | 18.826 | 0.725 | 29.617 | 1.00 | 51.06 |
| ATOM | 1821 | CB | ASP | 1064 | 20.028 | −0.059 | 30.257 | 1.00 | 56.05 |
| ATOM | 1822 | CG | ASP | 1064 | 19.702 | −1.545 | 30.444 | 1.00 | 60.81 |
| ATOM | 1823 | OD1 | ASP | 1064 | 18.725 | −1.865 | 31.141 | 1.00 | 63.43 |
| ATOM | 1824 | OD2 | ASP | 1064 | 20.446 | −2.368 | 29.899 | 1.00 | 63.06 |
| ATOM | 1825 | C | ASP | 1064 | 19.002 | 2.231 | 29.969 | 1.00 | 49.11 |
| ATOM | 1826 | O | ASP | 1064 | 19.413 | 2.601 | 31.078 | 1.00 | 49.97 |
| ATOM | 1827 | N | ALA | 1065 | 18.710 | 3.132 | 29.009 | 1.00 | 44.21 |
| ATOM | 1828 | H | ALA | 1065 | 18.384 | 2.830 | 28.133 | 1.00 | 0.00 |
| ATOM | 1829 | CA | ALA | 1065 | 18.844 | 4.583 | 29.220 | 1.00 | 38.56 |
| ATOM | 1830 | CB | ALA | 1065 | 19.775 | 5.181 | 28.158 | 1.00 | 36.16 |
| ATOM | 1831 | C | ALA | 1065 | 17.490 | 5.251 | 29.101 | 1.00 | 35.55 |
| ATOM | 1832 | O | ALA | 1065 | 16.635 | 4.782 | 28.352 | 1.00 | 35.47 |
| ATOM | 1833 | N | ARG | 1066 | 17.206 | 6.245 | 29.941 | 1.00 | 30.54 |
| ATOM | 1834 | H | ARG | 1066 | 17.865 | 6.479 | 30.622 | 1.00 | 0.00 |
| ATOM | 1835 | CA | ARG | 1066 | 15.936 | 6.985 | 29.823 | 1.00 | 27.55 |
| ATOM | 1836 | CB | ARG | 1066 | 15.347 | 7.230 | 31.253 | 1.00 | 25.84 |
| ATOM | 1837 | C | ARG | 1066 | 16.347 | 8.298 | 29.062 | 1.00 | 24.84 |
| ATOM | 1838 | O | ARG | 1066 | 17.048 | 9.170 | 29.585 | 1.00 | 23.59 |
| ATOM | 1839 | N | LEU | 1067 | 16.025 | 8.387 | 27.768 | 1.00 | 20.83 |
| ATOM | 1840 | H | LEU | 1067 | 15.576 | 7.616 | 27.375 | 1.00 | 0.00 |
| ATOM | 1841 | CA | LEU | 1067 | 16.400 | 9.511 | 26.911 | 1.00 | 18.67 |
| ATOM | 1842 | CB | LEU | 1067 | 16.818 | 8.933 | 25.600 | 1.00 | 17.20 |
| ATOM | 1843 | CG | LEU | 1067 | 17.879 | 7.828 | 25.715 | 1.00 | 18.31 |
| ATOM | 1844 | CD1 | LEU | 1067 | 18.157 | 7.336 | 24.301 | 1.00 | 18.81 |
| ATOM | 1845 | CD2 | LEU | 1067 | 19.166 | 8.315 | 26.388 | 1.00 | 19.05 |
| ATOM | 1846 | C | LEU | 1067 | 15.426 | 10.624 | 26.653 | 1.00 | 16.51 |
| ATOM | 1847 | O | LEU | 1067 | 14.263 | 10.307 | 26.483 | 1.00 | 17.65 |
| ATOM | 1848 | N | PRO | 1068 | 15.859 | 11.906 | 26.485 | 1.00 | 14.89 |
| ATOM | 1849 | CD | PRO | 1068 | 17.248 | 12.393 | 26.624 | 1.00 | 12.11 |
| ATOM | 1850 | CA | PRO | 1068 | 14.999 | 13.049 | 26.101 | 1.00 | 13.38 |
| ATOM | 1851 | CB | PRO | 1068 | 15.751 | 14.347 | 26.445 | 1.00 | 9.78 |
| ATOM | 1852 | CG | PRO | 1068 | 17.169 | 13.869 | 26.202 | 1.00 | 11.56 |
| ATOM | 1853 | C | PRO | 1068 | 14.662 | 12.991 | 24.614 | 1.00 | 13.13 |
| ATOM | 1854 | O | PRO | 1068 | 14.937 | 13.928 | 23.856 | 1.00 | 8.35 |
| ATOM | 1855 | N | LEU | 1069 | 13.916 | 11.923 | 24.252 | 1.00 | 14.63 |
| ATOM | 1856 | H | LEU | 1069 | 13.627 | 11.289 | 24.950 | 1.00 | 0.00 |
| ATOM | 1857 | CA | LEU | 1069 | 13.535 | 11.620 | 22.857 | 1.00 | 16.25 |
| ATOM | 1858 | CB | LEU | 1069 | 12.570 | 10.364 | 22.928 | 1.00 | 21.17 |
| ATOM | 1859 | CG | LEU | 1069 | 13.053 | 8.875 | 22.657 | 1.00 | 24.49 |
| ATOM | 1860 | CD1 | LEU | 1069 | 14.331 | 8.955 | 21.788 | 1.00 | 26.99 |
| ATOM | 1861 | CD2 | LEU | 1069 | 13.578 | 8.124 | 23.868 | 1.00 | 26.30 |
| ATOM | 1862 | C | LEU | 1069 | 12.942 | 12.758 | 21.974 | 1.00 | 13.96 |
| ATOM | 1863 | O | LEU | 1069 | 13.309 | 12.963 | 20.814 | 1.00 | 10.39 |
| ATOM | 1864 | N | LYS | 1070 | 12.064 | 13.577 | 22.549 | 1.00 | 11.37 |
| ATOM | 1865 | H | LYS | 1070 | 11.978 | 13.445 | 23.507 | 1.00 | 0.00 |
| ATOM | 1866 | CA | LYS | 1070 | 11.424 | 14.676 | 21.779 | 1.00 | 13.01 |
| ATOM | 1867 | CB | LYS | 1070 | 10.246 | 15.261 | 22.572 | 1.00 | 10.05 |
| ATOM | 1868 | CG | LYS | 1070 | 9.191 | 14.200 | 22.625 | 1.00 | 11.24 |
| ATOM | 1869 | CD | LYS | 1070 | 7.911 | 14.802 | 23.143 | 1.00 | 14.99 |
| ATOM | 1870 | CE | LYS | 1070 | 7.994 | 15.361 | 24.554 | 1.00 | 15.06 |
| ATOM | 1871 | NZ | LYS | 1070 | 6.614 | 15.692 | 24.997 | 1.00 | 18.74 |
| ATOM | 1872 | HZ1 | LYS | 1070 | 5.997 | 14.875 | 24.804 | 1.00 | 0.00 |
| ATOM | 1873 | HZ2 | LYS | 1070 | 6.278 | 16.518 | 24.459 | 1.00 | 0.00 |
| ATOM | 1874 | HZ3 | LYS | 1070 | 6.592 | 15.910 | 26.012 | 1.00 | 0.00 |
| ATOM | 1875 | C | LYS | 1070 | 12.317 | 15.847 | 21.368 | 1.00 | 11.97 |
| ATOM | 1876 | O | LYS | 1070 | 11.911 | 16.723 | 20.599 | 1.00 | 15.03 |
| ATOM | 1877 | N | TRP | 1071 | 13.503 | 15.856 | 21.956 | 1.00 | 11.60 |
| ATOM | 1878 | H | TRP | 1071 | 13.709 | 15.096 | 22.532 | 1.00 | 0.00 |
| ATOM | 1879 | CA | TRP | 1071 | 14.550 | 16.876 | 21.784 | 1.00 | 12.71 |
| ATOM | 1880 | CB | TRP | 1071 | 15.008 | 17.328 | 23.177 | 1.00 | 11.16 |
| ATOM | 1881 | CG | TRP | 1071 | 14.061 | 18.134 | 23.990 | 1.00 | 10.45 |
| ATOM | 1882 | CD2 | TRP | 1071 | 12.917 | 17.767 | 24.809 | 1.00 | 10.08 |
| ATOM | 1883 | CE2 | TRP | 1071 | 12.259 | 18.985 | 25.189 | 1.00 | 10.86 |
| ATOM | 1884 | CE3 | TRP | 1071 | 12.356 | 16.563 | 25.274 | 1.00 | 8.58 |
| ATOM | 1885 | CD1 | TRP | 1071 | 14.042 | 19.528 | 23.913 | 1.00 | 11.85 |
| ATOM | 1886 | NE1 | TRP | 1071 | 12.970 | 20.030 | 24.620 | 1.00 | 13.03 |
| ATOM | 1887 | HE1 | TRP | 1071 | 12.640 | 20.943 | 24.532 | 1.00 | 0.00 |
| ATOM | 1888 | CZ2 | TRP | 1071 | 11.094 | 18.981 | 25.981 | 1.00 | 5.11 |
| ATOM | 1889 | CZ3 | TRP | 1071 | 11.190 | 16.599 | 26.063 | 1.00 | 9.47 |
| ATOM | 1890 | CH2 | TRP | 1071 | 10.556 | 17.801 | 26.411 | 1.00 | 6.68 |
| ATOM | 1891 | C | TRP | 1071 | 15.743 | 16.343 | 21.017 | 1.00 | 15.21 |
| ATOM | 1892 | O | TRP | 1071 | 16.669 | 17.101 | 20.722 | 1.00 | 16.96 |
| ATOM | 1893 | N | MET | 1072 | 15.769 | 15.035 | 20.739 | 1.00 | 12.86 |
| ATOM | 1894 | H | MET | 1072 | 14.944 | 14.507 | 20.715 | 1.00 | 0.00 |
| ATOM | 1895 | CA | MET | 1072 | 16.934 | 14.484 | 20.049 | 1.00 | 13.80 |
| ATOM | 1896 | CB | MET | 1072 | 17.097 | 13.048 | 20.535 | 1.00 | 14.94 |
| ATOM | 1897 | CG | MET | 1072 | 17.573 | 13.061 | 21.970 | 1.00 | 14.27 |
| ATOM | 1898 | SD | MET | 1072 | 17.434 | 11.472 | 22.783 | 1.00 | 17.58 |
| ATOM | 1899 | CE | MET | 1072 | 18.672 | 10.380 | 21.891 | 1.00 | 14.32 |
| ATOM | 1900 | C | MET | 1072 | 16.970 | 14.508 | 18.544 | 1.00 | 13.02 |
| ATOM | 1901 | O | MET | 1072 | 15.992 | 14.191 | 17.881 | 1.00 | 14.04 |
| ATOM | 1902 | N | ALA | 1073 | 18.133 | 14.833 | 17.968 | 1.00 | 12.71 |
| ATOM | 1903 | H | ALA | 1073 | 18.779 | 15.326 | 18.507 | 1.00 | 0.00 |
| ATOM | 1904 | CA | ALA | 1073 | 18.258 | 14.804 | 16.512 | 1.00 | 11.73 |
| ATOM | 1905 | CB | ALA | 1073 | 19.656 | 15.355 | 16.127 | 1.00 | 9.43 |
| ATOM | 1906 | C | ALA | 1073 | 18.078 | 13.349 | 15.977 | 1.00 | 12.85 |
| ATOM | 1907 | O | ALA | 1073 | 18.489 | 12.371 | 16.643 | 1.00 | 10.82 |
| ATOM | 1908 | N | PRO | 1074 | 17.628 | 13.102 | 14.731 | 1.00 | 12.68 |
| ATOM | 1909 | CD | PRO | 1074 | 17.374 | 14.047 | 13.643 | 1.00 | 11.65 |
| ATOM | 1910 | CA | PRO | 1074 | 17.572 | 11.680 | 14.260 | 1.00 | 13.49 |
| ATOM | 1911 | CB | PRO | 1074 | 17.054 | 11.771 | 12.825 | 1.00 | 14.30 |
| ATOM | 1912 | CG | PRO | 1074 | 16.586 | 13.217 | 12.622 | 1.00 | 13.69 |
| ATOM | 1913 | C | PRO | 1074 | 18.921 | 10.926 | 14.371 | 1.00 | 12.98 |
| ATOM | 1914 | O | PRO | 1074 | 18.954 | 9.748 | 14.733 | 1.00 | 14.35 |
| ATOM | 1915 | N | GLU | 1075 | 20.076 | 11.512 | 14.037 | 1.00 | 12.92 |
| ATOM | 1916 | H | GLU | 1075 | 20.026 | 12.303 | 13.470 | 1.00 | 0.00 |
| ATOM | 1917 | CA | GLU | 1075 | 21.348 | 10.761 | 14.215 | 1.00 | 13.66 |
| ATOM | 1918 | CB | GLU | 1075 | 22.591 | 11.534 | 13.701 | 1.00 | 13.29 |
| ATOM | 1919 | CG | GLU | 1075 | 23.015 | 12.810 | 14.455 | 1.00 | 11.21 |
| ATOM | 1920 | CD | GLU | 1075 | 22.234 | 14.060 | 14.099 | 1.00 | 10.28 |
| ATOM | 1921 | OE1 | GLU | 1075 | 21.173 | 13.988 | 13.476 | 1.00 | 13.07 |
| ATOM | 1922 | OE2 | GLU | 1075 | 22.672 | 15.135 | 14.482 | 1.00 | 10.97 |
| ATOM | 1923 | C | GLU | 1075 | 21.585 | 10.430 | 15.679 | 1.00 | 14.82 |
| ATOM | 1924 | O | GLU | 1075 | 22.246 | 9.432 | 16.026 | 1.00 | 16.29 |
| ATOM | 1925 | N | THR | 1076 | 21.046 | 11.232 | 16.633 | 1.00 | 15.92 |
| ATOM | 1926 | H | THR | 1076 | 20.521 | 12.020 | 16.389 | 1.00 | 0.00 |
| ATOM | 1927 | CA | THR | 1076 | 21.253 | 10.849 | 18.072 | 1.00 | 15.21 |
| ATOM | 1928 | CB | THR | 1076 | 21.006 | 12.043 | 19.093 | 1.00 | 13.64 |
| ATOM | 1929 | OG1 | THR | 1076 | 21.788 | 13.186 | 18.635 | 1.00 | 13.03 |
| ATOM | 1930 | HG1 | THR | 1076 | 22.087 | 13.683 | 19.400 | 1.00 | 0.00 |
| ATOM | 1931 | CG2 | THR | 1076 | 21.496 | 11.741 | 20.511 | 1.00 | 9.08 |
| ATOM | 1932 | C | THR | 1076 | 20.336 | 9.682 | 18.425 | 1.00 | 14.26 |
| ATOM | 1933 | O | THR | 1076 | 20.774 | 8.708 | 19.041 | 1.00 | 15.48 |
| ATOM | 1934 | N | ILE | 1077 | 19.077 | 9.714 | 18.029 | 1.00 | 13.74 |
| ATOM | 1935 | H | ILE | 1077 | 18.728 | 10.519 | 17.583 | 1.00 | 0.00 |
| ATOM | 1936 | CA | ILE | 1077 | 18.163 | 8.606 | 18.337 | 1.00 | 13.70 |
| ATOM | 1937 | CB | ILE | 1077 | 16.715 | 8.927 | 17.836 | 1.00 | 14.40 |
| ATOM | 1938 | CG2 | ILE | 1077 | 15.838 | 7.653 | 18.007 | 1.00 | 13.38 |
| ATOM | 1939 | CG1 | ILE | 1077 | 16.060 | 10.101 | 18.606 | 1.00 | 13.41 |
| ATOM | 1940 | CD1 | ILE | 1077 | 14.780 | 10.418 | 17.804 | 1.00 | 10.69 |
| ATOM | 1941 | C | ILE | 1077 | 18.668 | 7.330 | 17.618 | 1.00 | 14.77 |
| ATOM | 1942 | O | ILE | 1077 | 18.819 | 6.261 | 18.217 | 1.00 | 16.01 |
| ATOM | 1943 | N | PHE | 1078 | 18.908 | 7.424 | 16.305 | 1.00 | 14.97 |
| ATOM | 1944 | H | PHE | 1078 | 18.715 | 8.276 | 15.875 | 1.00 | 0.00 |
| ATOM | 1945 | CA | PHE | 1078 | 19.325 | 6.288 | 15.487 | 1.00 | 16.52 |
| ATOM | 1946 | CB | PHE | 1078 | 18.873 | 6.586 | 14.027 | 1.00 | 18.97 |
| ATOM | 1947 | CG | PHE | 1078 | 17.399 | 6.742 | 13.944 | 1.00 | 22.87 |
| ATOM | 1948 | CD1 | PHE | 1078 | 16.813 | 7.998 | 13.796 | 1.00 | 24.14 |
| ATOM | 1949 | CD2 | PHE | 1078 | 16.558 | 5.618 | 14.057 | 1.00 | 25.73 |
| ATOM | 1950 | CE1 | PHE | 1078 | 15.414 | 8.134 | 13.771 | 1.00 | 24.81 |
| ATOM | 1951 | CE2 | PHE | 1078 | 15.148 | 5.758 | 14.027 | 1.00 | 25.55 |
| ATOM | 1952 | CZ | PHE | 1078 | 14.591 | 7.023 | 13.883 | 1.00 | 21.74 |
| ATOM | 1953 | C | PHE | 1078 | 20.800 | 5.905 | 15.542 | 1.00 | 17.56 |
| ATOM | 1954 | O | PHE | 1078 | 21.177 | 4.735 | 15.442 | 1.00 | 16.98 |
| ATOM | 1955 | N | ASP | 1079 | 21.683 | 6.865 | 15.660 | 1.00 | 14.61 |
| ATOM | 1956 | H | ASP | 1079 | 21.384 | 7.790 | 15.708 | 1.00 | 0.00 |
| ATOM | 1957 | CA | ASP | 1079 | 23.071 | 6.433 | 15.664 | 1.00 | 16.88 |
| ATOM | 1958 | CB | ASP | 1079 | 23.738 | 7.032 | 14.462 | 1.00 | 16.01 |
| ATOM | 1959 | CG | ASP | 1079 | 22.964 | 6.674 | 13.173 | 1.00 | 20.95 |
| ATOM | 1960 | OD1 | ASP | 1079 | 22.252 | 5.653 | 13.123 | 1.00 | 21.53 |
| ATOM | 1961 | OD2 | ASP | 1079 | 23.109 | 7.443 | 12.213 | 1.00 | 23.80 |
| ATOM | 1962 | C | ASP | 1079 | 23.872 | 6.735 | 16.915 | 1.00 | 18.01 |
| ATOM | 1963 | O | ASP | 1079 | 25.084 | 6.453 | 16.973 | 1.00 | 19.68 |
| ATOM | 1964 | N | ARG | 1080 | 23.180 | 7.195 | 17.967 | 1.00 | 16.85 |
| ATOM | 1965 | H | ARG | 1080 | 22.206 | 7.316 | 17.874 | 1.00 | 0.00 |
| ATOM | 1966 | CA | ARG | 1080 | 23.877 | 7.532 | 19.234 | 1.00 | 16.62 |
| ATOM | 1967 | CB | ARG | 1080 | 24.433 | 6.241 | 19.930 | 1.00 | 18.49 |
| ATOM | 1968 | CG | ARG | 1080 | 23.265 | 5.327 | 20.241 | 1.00 | 26.09 |
| ATOM | 1969 | CD | ARG | 1080 | 23.665 | 4.127 | 21.129 | 1.00 | 36.59 |
| ATOM | 1970 | NE | ARG | 1080 | 24.602 | 3.227 | 20.408 | 1.00 | 44.61 |
| ATOM | 1971 | HE | ARG | 1080 | 25.049 | 3.580 | 19.614 | 1.00 | 0.00 |
| ATOM | 1972 | CZ | ARG | 1080 | 24.791 | 1.942 | 20.750 | 1.00 | 46.46 |
| ATOM | 1973 | NH1 | ARG | 1080 | 25.637 | 1.188 | 20.066 | 1.00 | 47.71 |
| ATOM | 1974 | HH11 | ARG | 1080 | 26.140 | 1.588 | 19.302 | 1.00 | 0.00 |
| ATOM | 1975 | HH12 | ARG | 1080 | 25.786 | 0.232 | 20.322 | 1.00 | 0.00 |
| ATOM | 1976 | NH2 | ARG | 1080 | 24.123 | 1.397 | 21.761 | 1.00 | 51.34 |
| ATOM | 1977 | HH21 | ARG | 1080 | 23.445 | 1.932 | 22.265 | 1.00 | 0.00 |
| ATOM | 1978 | HH22 | ARG | 1080 | 24.284 | 0.441 | 22.007 | 1.00 | 0.00 |
| ATOM | 1979 | C | ARG | 1080 | 25.007 | 8.525 | 19.013 | 1.00 | 13.24 |
| ATOM | 1980 | O | ARG | 1080 | 25.958 | 8.562 | 19.757 | 1.00 | 12.37 |
| ATOM | 1981 | N | VAL | 1081 | 24.907 | 9.325 | 17.962 | 1.00 | 12.79 |
| ATOM | 1982 | H | VAL | 1081 | 24.083 | 9.269 | 17.417 | 1.00 | 0.00 |
| ATOM | 1983 | CA | VAL | 1081 | 25.874 | 10.372 | 17.635 | 1.00 | 12.63 |
| ATOM | 1984 | CB | VAL | 1081 | 25.968 | 10.510 | 16.073 | 1.00 | 13.26 |
| ATOM | 1985 | CG1 | VAL | 1081 | 26.648 | 11.775 | 15.527 | 1.00 | 10.26 |
| ATOM | 1986 | CG2 | VAL | 1081 | 26.799 | 9.315 | 15.662 | 1.00 | 11.69 |
| ATOM | 1987 | C | VAL | 1081 | 25.420 | 11.683 | 18.280 | 1.00 | 12.66 |
| ATOM | 1988 | O | VAL | 1081 | 24.316 | 12.183 | 18.060 | 1.00 | 11.40 |
| ATOM | 1989 | N | TYR | 1082 | 26.288 | 12.308 | 19.040 | 1.00 | 11.87 |
| ATOM | 1990 | H | TYR | 1082 | 27.188 | 11.920 | 19.082 | 1.00 | 0.00 |
| ATOM | 1991 | CA | TYR | 1082 | 26.063 | 13.601 | 19.756 | 1.00 | 11.71 |
| ATOM | 1992 | CB | TYR | 1082 | 26.309 | 13.541 | 21.287 | 1.00 | 11.27 |
| ATOM | 1993 | CG | TYR | 1082 | 25.315 | 12.691 | 21.954 | 1.00 | 15.85 |
| ATOM | 1994 | CD1 | TYR | 1082 | 25.377 | 11.276 | 21.911 | 1.00 | 16.88 |
| ATOM | 1995 | CE1 | TYR | 1082 | 24.346 | 10.511 | 22.499 | 1.00 | 17.30 |
| ATOM | 1996 | CD2 | TYR | 1082 | 24.240 | 13.343 | 22.581 | 1.00 | 16.48 |
| ATOM | 1997 | CE2 | TYR | 1082 | 23.222 | 12.589 | 23.160 | 1.00 | 14.83 |
| ATOM | 1998 | CZ | TYR | 1082 | 23.278 | 11.197 | 23.111 | 1.00 | 16.91 |
| ATOM | 1999 | OH | TYR | 1082 | 22.185 | 10.525 | 23.600 | 1.00 | 19.99 |
| ATOM | 2000 | HH | TYR | 1082 | 22.283 | 9.580 | 23.453 | 1.00 | 0.00 |
| ATOM | 2001 | C | TYR | 1082 | 27.091 | 14.611 | 19.260 | 1.00 | 13.24 |
| ATOM | 2002 | O | TYR | 1082 | 28.296 | 14.400 | 19.430 | 1.00 | 15.09 |
| ATOM | 2003 | N | THR | 1083 | 26.707 | 15.712 | 18.610 | 1.00 | 12.75 |
| ATOM | 2004 | H | THR | 1083 | 25.759 | 15.908 | 18.542 | 1.00 | 0.00 |
| ATOM | 2005 | CA | THR | 1083 | 27.681 | 16.752 | 18.117 | 1.00 | 10.58 |
| ATOM | 2006 | CB | THR | 1083 | 27.956 | 16.629 | 16.576 | 1.00 | 10.00 |
| ATOM | 2007 | OG1 | THR | 1083 | 26.827 | 17.185 | 15.954 | 1.00 | 12.12 |
| ATOM | 2008 | HG1 | THR | 1083 | 26.897 | 16.948 | 15.006 | 1.00 | 0.00 |
| ATOM | 2009 | CG2 | THR | 1083 | 28.208 | 15.168 | 16.052 | 1.00 | 9.41 |
| ATOM | 2010 | C | THR | 1083 | 27.033 | 18.123 | 18.387 | 1.00 | 9.34 |
| ATOM | 2011 | O | THR | 1083 | 25.851 | 18.142 | 18.723 | 1.00 | 12.45 |
| ATOM | 2012 | N | ILE | 1084 | 27.677 | 19.300 | 18.334 | 1.00 | 11.06 |
| ATOM | 2013 | H | ILE | 1084 | 28.656 | 19.323 | 18.275 | 1.00 | 0.00 |
| ATOM | 2014 | CA | ILE | 1084 | 26.889 | 20.540 | 18.600 | 1.00 | 10.56 |
| ATOM | 2015 | CB | ILE | 1084 | 27.555 | 21.999 | 18.525 | 1.00 | 12.56 |
| ATOM | 2016 | CG2 | ILE | 1084 | 27.662 | 22.699 | 19.946 | 1.00 | 10.69 |
| ATOM | 2017 | CG1 | ILE | 1084 | 28.767 | 21.818 | 17.686 | 1.00 | 13.36 |
| ATOM | 2018 | CD1 | ILE | 1084 | 28.574 | 21.382 | 16.218 | 1.00 | 11.18 |
| ATOM | 2019 | C | ILE | 1084 | 25.849 | 20.644 | 17.557 | 1.00 | 11.16 |
| ATOM | 2020 | O | ILE | 1084 | 24.934 | 21.418 | 17.780 | 1.00 | 11.43 |
| ATOM | 2021 | N | GLN | 1085 | 25.946 | 19.919 | 16.401 | 1.00 | 12.75 |
| ATOM | 2022 | H | GLN | 1085 | 26.743 | 19.382 | 16.224 | 1.00 | 0.00 |
| ATOM | 2023 | CA | GLN | 1085 | 24.864 | 20.027 | 15.398 | 1.00 | 12.31 |
| ATOM | 2024 | CB | GLN | 1085 | 25.332 | 19.526 | 13.973 | 1.00 | 13.55 |
| ATOM | 2025 | CG | GLN | 1085 | 26.324 | 20.568 | 13.359 | 1.00 | 11.15 |
| ATOM | 2026 | CD | GLN | 1085 | 25.864 | 22.045 | 13.386 | 1.00 | 8.99 |
| ATOM | 2027 | OE1 | GLN | 1085 | 26.287 | 22.855 | 14.196 | 1.00 | 10.88 |
| ATOM | 2028 | NE2 | GLN | 1085 | 24.986 | 22.465 | 12.499 | 1.00 | 8.06 |
| ATOM | 2029 | HE21 | GLN | 1085 | 24.614 | 21.886 | 11.812 | 1.00 | 0.00 |
| ATOM | 2030 | HE22 | GLN | 1085 | 24.725 | 23.411 | 12.552 | 1.00 | 0.00 |
| ATOM | 2031 | C | GLN | 1085 | 23.665 | 19.271 | 15.889 | 1.00 | 12.53 |
| ATOM | 2032 | O | GLN | 1085 | 22.527 | 19.613 | 15.561 | 1.00 | 14.94 |
| ATOM | 2033 | N | SER | 1086 | 23.853 | 18.210 | 16.672 | 1.00 | 11.94 |
| ATOM | 2034 | H | SER | 1086 | 24.747 | 17.958 | 16.970 | 1.00 | 0.00 |
| ATOM | 2035 | CA | SER | 1086 | 22.631 | 17.564 | 17.224 | 1.00 | 12.65 |
| ATOM | 2036 | CB | SER | 1086 | 22.920 | 16.111 | 17.756 | 1.00 | 11.86 |
| ATOM | 2037 | OG | SER | 1086 | 23.885 | 16.019 | 18.783 | 1.00 | 13.25 |
| ATOM | 2038 | HG | SER | 1086 | 24.012 | 15.070 | 18.951 | 1.00 | 0.00 |
| ATOM | 2039 | C | SER | 1086 | 22.137 | 18.524 | 18.335 | 1.00 | 12.90 |
| ATOM | 2040 | O | SER | 1086 | 20.930 | 18.652 | 18.570 | 1.00 | 11.69 |
| ATOM | 2041 | N | ASP | 1087 | 23.043 | 19.250 | 19.045 | 1.00 | 12.13 |
| ATOM | 2042 | H | ASP | 1087 | 23.982 | 18.976 | 19.028 | 1.00 | 0.00 |
| ATOM | 2043 | CA | ASP | 1087 | 22.540 | 20.283 | 20.013 | 1.00 | 10.80 |
| ATOM | 2044 | CB | ASP | 1087 | 23.651 | 21.007 | 20.814 | 1.00 | 12.57 |
| ATOM | 2045 | CG | ASP | 1087 | 24.286 | 20.205 | 21.949 | 1.00 | 10.95 |
| ATOM | 2046 | OD1 | ASP | 1087 | 25.324 | 20.621 | 22.403 | 1.00 | 10.20 |
| ATOM | 2047 | OD2 | ASP | 1087 | 23.762 | 19.209 | 22.413 | 1.00 | 13.27 |
| ATOM | 2048 | C | ASP | 1087 | 21.796 | 21.387 | 19.233 | 1.00 | 11.87 |
| ATOM | 2049 | O | ASP | 1087 | 20.933 | 22.063 | 19.790 | 1.00 | 12.59 |
| ATOM | 2050 | N | VAL | 1088 | 22.119 | 21.676 | 17.933 | 1.00 | 9.83 |
| ATOM | 2051 | H | VAL | 1088 | 22.958 | 21.328 | 17.562 | 1.00 | 0.00 |
| ATOM | 2052 | CA | VAL | 1088 | 21.335 | 22.689 | 17.151 | 1.00 | 9.60 |
| ATOM | 2053 | CB | VAL | 1088 | 21.994 | 23.020 | 15.747 | 1.00 | 8.88 |
| ATOM | 2054 | CG1 | VAL | 1088 | 20.956 | 23.738 | 14.845 | 1.00 | 4.87 |
| ATOM | 2055 | CG2 | VAL | 1088 | 23.275 | 23.932 | 15.982 | 1.00 | 5.03 |
| ATOM | 2056 | C | VAL | 1088 | 19.917 | 22.137 | 16.947 | 1.00 | 11.41 |
| ATOM | 2057 | O | VAL | 1088 | 18.924 | 22.867 | 17.147 | 1.00 | 11.23 |
| ATOM | 2058 | N | TRP | 1089 | 19.786 | 20.828 | 16.621 | 1.00 | 10.88 |
| ATOM | 2059 | H | TRP | 1089 | 20.582 | 20.310 | 16.377 | 1.00 | 0.00 |
| ATOM | 2060 | CA | TRP | 1089 | 18.435 | 20.245 | 16.505 | 1.00 | 11.12 |
| ATOM | 2061 | CB | TRP | 1089 | 18.486 | 18.681 | 16.227 | 1.00 | 11.02 |
| ATOM | 2062 | CG | TRP | 1089 | 17.170 | 17.980 | 16.077 | 1.00 | 8.30 |
| ATOM | 2063 | CD2 | TRP | 1089 | 16.466 | 17.518 | 14.926 | 1.00 | 6.33 |
| ATOM | 2064 | CE2 | TRP | 1089 | 15.175 | 17.119 | 15.342 | 1.00 | 7.41 |
| ATOM | 2065 | CE3 | TRP | 1089 | 16.803 | 17.397 | 13.569 | 1.00 | 8.47 |
| ATOM | 2066 | CD1 | TRP | 1089 | 16.238 | 17.805 | 17.123 | 1.00 | 12.22 |
| ATOM | 2067 | NE1 | TRP | 1089 | 15.049 | 17.299 | 16.690 | 1.00 | 8.53 |
| ATOM | 2068 | HE1 | TRP | 1089 | 14.286 | 17.020 | 17.253 | 1.00 | 0.00 |
| ATOM | 2069 | CZ2 | TRP | 1089 | 14.238 | 16.631 | 14.445 | 1.00 | 5.85 |
| ATOM | 2070 | CZ3 | TRP | 1089 | 15.874 | 16.901 | 12.659 | 1.00 | 9.30 |
| ATOM | 2071 | CH2 | TRP | 1089 | 14.604 | 16.530 | 13.110 | 1.00 | 11.51 |
| ATOM | 2072 | C | TRP | 1089 | 17.687 | 20.484 | 17.846 | 1.00 | 12.38 |
| ATOM | 2073 | O | TRP | 1089 | 16.548 | 20.958 | 17.822 | 1.00 | 14.30 |
| ATOM | 2074 | N | SER | 1090 | 18.284 | 20.174 | 19.025 | 1.00 | 10.64 |
| ATOM | 2075 | H | SER | 1090 | 19.139 | 19.685 | 18.992 | 1.00 | 0.00 |
| ATOM | 2076 | CA | SER | 1090 | 17.603 | 20.377 | 20.344 | 1.00 | 9.63 |
| ATOM | 2077 | CB | SER | 1090 | 18.468 | 19.952 | 21.562 | 1.00 | 7.41 |
| ATOM | 2078 | OG | SER | 1090 | 18.882 | 18.578 | 21.442 | 1.00 | 9.63 |
| ATOM | 2079 | HG | SER | 1090 | 18.517 | 18.113 | 22.202 | 1.00 | 0.00 |
| ATOM | 2080 | C | SER | 1090 | 17.254 | 21.837 | 20.547 | 1.00 | 10.38 |
| ATOM | 2081 | O | SER | 1090 | 16.212 | 22.170 | 21.148 | 1.00 | 10.77 |
| ATOM | 2082 | N | PHE | 1091 | 18.135 | 22.759 | 20.081 | 1.00 | 9.58 |
| ATOM | 2083 | H | PHE | 1091 | 18.984 | 22.446 | 19.696 | 1.00 | 0.00 |
| ATOM | 2084 | CA | PHE | 1091 | 17.859 | 24.202 | 20.192 | 1.00 | 8.97 |
| ATOM | 2085 | CB | PHE | 1091 | 19.106 | 24.977 | 19.703 | 1.00 | 7.47 |
| ATOM | 2086 | CG | PHE | 1091 | 18.882 | 26.417 | 19.842 | 1.00 | 9.60 |
| ATOM | 2087 | CD1 | PHE | 1091 | 18.986 | 27.042 | 21.094 | 1.00 | 9.17 |
| ATOM | 2088 | CD2 | PHE | 1091 | 18.551 | 27.161 | 18.675 | 1.00 | 10.45 |
| ATOM | 2089 | GE1 | PHE | 1091 | 18.758 | 28.407 | 21.165 | 1.00 | 7.18 |
| ATOM | 2090 | CE2 | PHE | 1091 | 18.326 | 28.533 | 18.772 | 1.00 | 7.74 |
| ATOM | 2091 | CZ | PHE | 1091 | 18.438 | 29.149 | 20.030 | 1.00 | 7.77 |
| ATOM | 2092 | C | PHE | 1091 | 16.592 | 24.521 | 19.368 | 1.00 | 9.75 |
| ATOM | 2093 | O | PHE | 1091 | 15.770 | 25.352 | 19.780 | 1.00 | 10.24 |
| ATOM | 2094 | N | GLY | 1092 | 16.339 | 23.897 | 18.202 | 1.00 | 9.03 |
| ATOM | 2095 | H | GLY | 1092 | 17.019 | 23.300 | 17.823 | 1.00 | 0.00 |
| ATOM | 2096 | CA | GLY | 1092 | 15.077 | 24.202 | 17.450 | 1.00 | 6.29 |
| ATOM | 2097 | C | GLY | 1092 | 13.879 | 23.763 | 18.284 | 1.00 | 6.25 |
| ATOM | 2098 | O | GLY | 1092 | 12.802 | 24.356 | 18.270 | 1.00 | 9.11 |
| ATOM | 2099 | N | VAL | 1093 | 13.977 | 22.663 | 19.009 | 1.00 | 7.63 |
| ATOM | 2100 | H | VAL | 1093 | 14.794 | 22.125 | 18.950 | 1.00 | 0.00 |
| ATOM | 2101 | CA | VAL | 1093 | 12.845 | 22.204 | 19.873 | 1.00 | 10.13 |
| ATOM | 2102 | CB | VAL | 1093 | 13.067 | 20.719 | 20.459 | 1.00 | 11.01 |
| ATOM | 2103 | CG1 | VAL | 1093 | 11.863 | 20.352 | 21.343 | 1.00 | 8.45 |
| ATOM | 2104 | CG2 | VAL | 1093 | 13.263 | 19.691 | 19.319 | 1.00 | 8.63 |
| ATOM | 2105 | C | VAL | 1093 | 12.681 | 23.204 | 21.060 | 1.00 | 11.32 |
| ATOM | 2106 | O | VAL | 1093 | 11.559 | 23.487 | 21.474 | 1.00 | 14.17 |
| ATOM | 2107 | N | LEU | 1094 | 13.772 | 23.747 | 21.627 | 1.00 | 9.67 |
| ATOM | 2108 | H | LEU | 1094 | 14.635 | 23.394 | 21.322 | 1.00 | 0.00 |
| ATOM | 2109 | CA | LEU | 1094 | 13.742 | 24.765 | 22.712 | 1.00 | 10.28 |
| ATOM | 2110 | CB | LEU | 1094 | 15.255 | 25.024 | 23.209 | 1.00 | 10.87 |
| ATOM | 2111 | CG | LEU | 1094 | 15.700 | 26.051 | 24.310 | 1.00 | 12.46 |
| ATOM | 2112 | CD1 | LEU | 1094 | 15.843 | 27.424 | 23.741 | 1.00 | 11.93 |
| ATOM | 2113 | CD2 | LEU | 1094 | 14.599 | 26.435 | 25.245 | 1.00 | 15.11 |
| ATOM | 2114 | C | LEU | 1094 | 13.044 | 26.000 | 22.135 | 1.00 | 9.48 |
| ATOM | 2115 | O | LEU | 1094 | 12.128 | 26.502 | 22.771 | 1.00 | 10.99 |
| ATOM | 2116 | N | LEU | 1095 | 13.350 | 26.473 | 20.919 | 1.00 | 7.46 |
| ATOM | 2117 | H | LEU | 1095 | 14.104 | 26.066 | 20.447 | 1.00 | 0.00 |
| ATOM | 2118 | CA | LEU | 1095 | 12.633 | 27.625 | 20.311 | 1.00 | 9.94 |
| ATOM | 2119 | CB | LEU | 1095 | 13.046 | 27.856 | 18.830 | 1.00 | 8.07 |
| ATOM | 2120 | CG | LEU | 1095 | 14.478 | 28.288 | 18.696 | 1.00 | 8.54 |
| ATOM | 2121 | CD1 | LEU | 1095 | 14.818 | 28.426 | 17.233 | 1.00 | 6.72 |
| ATOM | 2122 | CD2 | LEU | 1095 | 14.692 | 29.599 | 19.443 | 1.00 | 8.82 |
| ATOM | 2123 | C | LEU | 1095 | 11.116 | 27.333 | 20.293 | 1.00 | 10.78 |
| ATOM | 2124 | O | LEU | 1095 | 10.269 | 28.173 | 20.649 | 1.00 | 10.35 |
| ATOM | 2125 | N | TRP | 1096 | 10.744 | 26.085 | 19.914 | 1.00 | 10.43 |
| ATOM | 2126 | H | TRP | 1096 | 11.417 | 25.461 | 19.560 | 1.00 | 0.00 |
| ATOM | 2127 | CA | TRP | 1096 | 9.322 | 25.709 | 19.884 | 1.00 | 8.61 |
| ATOM | 2128 | CB | TRP | 1096 | 9.118 | 24.244 | 19.447 | 1.00 | 7.55 |
| ATOM | 2129 | CG | TRP | 1096 | 7.661 | 23.946 | 19.136 | 1.00 | 6.18 |
| ATOM | 2130 | CD2 | TRP | 1096 | 6.731 | 23.463 | 20.055 | 1.00 | 6.42 |
| ATOM | 2131 | CE2 | TRP | 1096 | 5.494 | 23.275 | 19.334 | 1.00 | 8.76 |
| ATOM | 2132 | CE3 | TRP | 1096 | 6.849 | 23.170 | 21.430 | 1.00 | 8.41 |
| ATOM | 2133 | CD1 | TRP | 1096 | 6.939 | 24.040 | 17.890 | 1.00 | 6.35 |
| ATOM | 2134 | NE1 | TRP | 1096 | 5.640 | 23.634 | 18.015 | 1.00 | 8.72 |
| ATOM | 2135 | HE1 | TRP | 1096 | 4.973 | 23.601 | 17.286 | 1.00 | 0.00 |
| ATOM | 2136 | CZ2 | TRP | 1096 | 4.392 | 22.783 | 20.055 | 1.00 | 8.56 |
| ATOM | 2137 | CZ3 | TRP | 1096 | 5.751 | 22.688 | 22.130 | 1.00 | 5.90 |
| ATOM | 2138 | CH2 | TRP | 1096 | 4.543 | 22.500 | 21.446 | 1.00 | 8.01 |
| ATOM | 2139 | C | TRP | 1096 | 8.729 | 25.848 | 21.279 | 1.00 | 9.79 |
| ATOM | 2140 | O | TRP | 1096 | 7.596 | 26.252 | 21.459 | 1.00 | 12.59 |
| ATOM | 2141 | N | GLU | 1097 | 9.463 | 25.449 | 22.314 | 1.00 | 9.28 |
| ATOM | 2142 | H | GLU | 1097 | 10.306 | 25.001 | 22.090 | 1.00 | 0.00 |
| ATOM | 2143 | CA | GLU | 1097 | 9.023 | 25.545 | 23.700 | 1.00 | 8.78 |
| ATOM | 2144 | CB | GLU | 1097 | 10.061 | 24.866 | 24.651 | 1.00 | 8.41 |
| ATOM | 2145 | CG | GLU | 1097 | 10.120 | 23.346 | 24.435 | 1.00 | 6.82 |
| ATOM | 2146 | CD | GLU | 1097 | 11.055 | 22.802 | 25.470 | 1.00 | 7.77 |
| ATOM | 2147 | OE1 | GLU | 1097 | 10.602 | 22.418 | 26.524 | 1.00 | 9.18 |
| ATOM | 2148 | OE2 | GLU | 1097 | 12.252 | 22.807 | 25.273 | 1.00 | 10.48 |
| ATOM | 2149 | C | GLU | 1097 | 8.847 | 26.984 | 24.086 | 1.00 | 8.43 |
| ATOM | 2150 | O | GLU | 1097 | 7.894 | 27.359 | 24.751 | 1.00 | 6.16 |
| ATOM | 2151 | N | ILE | 1098 | 9.820 | 27.839 | 23.734 | 1.00 | 11.37 |
| ATOM | 2152 | H | ILE | 1098 | 10.604 | 27.500 | 23.266 | 1.00 | 0.00 |
| ATOM | 2153 | CA | ILE | 1098 | 9.712 | 29.280 | 24.036 | 1.00 | 8.81 |
| ATOM | 2154 | CB | ILE | 1098 | 11.010 | 29.989 | 23.612 | 1.00 | 6.80 |
| ATOM | 2155 | CG2 | ILE | 1098 | 10.773 | 31.508 | 23.698 | 1.00 | 6.09 |
| ATOM | 2156 | CG1 | ILE | 1098 | 12.152 | 29.549 | 24.514 | 1.00 | 8.57 |
| ATOM | 2157 | CD1 | ILE | 1098 | 13.465 | 30.147 | 23.985 | 1.00 | 6.22 |
| ATOM | 2158 | C | ILE | 1098 | 8.478 | 29.936 | 23.346 | 1.00 | 8.62 |
| ATOM | 2159 | O | ILE | 1098 | 7.685 | 30.612 | 24.022 | 1.00 | 6.94 |
| ATOM | 2160 | N | PHE | 1099 | 8.297 | 29.730 | 22.015 | 1.00 | 6.87 |
| ATOM | 2161 | H | PHE | 1099 | 8.967 | 29.207 | 21.530 | 1.00 | 0.00 |
| ATOM | 2162 | CA | PHE | 1099 | 7.182 | 30.328 | 21.301 | 1.00 | 6.82 |
| ATOM | 2163 | CB | PHE | 1099 | 7.574 | 30.540 | 19.811 | 1.00 | 6.09 |
| ATOM | 2164 | CG | PHE | 1099 | 8.639 | 31.568 | 19.785 | 1.00 | 6.80 |
| ATOM | 2165 | CD1 | PHE | 1099 | 10.010 | 31.207 | 19.929 | 1.00 | 6.30 |
| ATOM | 2166 | CD2 | PHE | 1099 | 8.297 | 32.937 | 19.682 | 1.00 | 8.05 |
| ATOM | 2167 | CE1 | PHE | 1099 | 11.028 | 32.170 | 19.986 | 1.00 | 7.24 |
| ATOM | 2168 | CE2 | PHE | 1099 | 9.329 | 33.915 | 19.734 | 1.00 | 8.14 |
| ATOM | 2169 | CZ | PHE | 1099 | 10.677 | 33.534 | 19.891 | 1.00 | 6.46 |
| ATOM | 2170 | C | PHE | 1099 | 5.829 | 29.620 | 21.415 | 1.00 | 8.68 |
| ATOM | 2171 | O | PHE | 1099 | 4.861 | 29.920 | 20.732 | 1.00 | 11.03 |
| ATOM | 2172 | N | SER | 1100 | 5.726 | 28.631 | 22.276 | 1.00 | 10.87 |
| ATOM | 2173 | H | SER | 1100 | 6.580 | 28.265 | 22.599 | 1.00 | 0.00 |
| ATOM | 2174 | CA | SER | 1100 | 4.495 | 27.899 | 22.676 | 1.00 | 9.16 |
| ATOM | 2175 | CB | SER | 1100 | 4.635 | 26.327 | 22.565 | 1.00 | 8.92 |
| ATOM | 2176 | OG | SER | 1100 | 5.586 | 25.870 | 23.533 | 1.00 | 11.04 |
| ATOM | 2177 | HG | SER | 1100 | 5.920 | 24.995 | 23.293 | 1.00 | 0.00 |
| ATOM | 2178 | C | SER | 1100 | 4.261 | 28.232 | 24.173 | 1.00 | 9.51 |
| ATOM | 2179 | O | SER | 1100 | 3.289 | 27.773 | 24.757 | 1.00 | 10.70 |
| ATOM | 2180 | N | LEU | 1101 | 5.077 | 29.127 | 24.789 | 1.00 | 9.56 |
| ATOM | 2181 | H | LEU | 1101 | 5.744 | 29.557 | 24.218 | 1.00 | 0.00 |
| ATOM | 2182 | CA | LEU | 1101 | 5.086 | 29.543 | 26.191 | 1.00 | 8.07 |
| ATOM | 2183 | CB | LEU | 1101 | 3.916 | 30.481 | 26.532 | 1.00 | 8.04 |
| ATOM | 2184 | CG | LEU | 1101 | 3.982 | 31.895 | 25.920 | 1.00 | 8.66 |
| ATOM | 2185 | CD1 | LEU | 1101 | 2.834 | 32.782 | 26.539 | 1.00 | 6.34 |
| ATOM | 2186 | CD2 | LEU | 1101 | 5.327 | 32.586 | 26.269 | 1.00 | 7.60 |
| ATOM | 2187 | C | LEU | 1101 | 5.067 | 28.346 | 27.100 | 1.00 | 9.80 |
| ATOM | 2188 | O | LEU | 1101 | 4.172 | 28.005 | 27.845 | 1.00 | 9.48 |
| ATOM | 2189 | N | GLY | 1102 | 6.129 | 27.603 | 26.998 | 1.00 | 11.45 |
| ATOM | 2190 | H | GLY | 1102 | 6.780 | 27.818 | 26.300 | 1.00 | 0.00 |
| ATOM | 2191 | CA | GLY | 1102 | 6.378 | 26.409 | 27.794 | 1.00 | 11.15 |
| ATOM | 2192 | C | GLY | 1102 | 5.528 | 25.197 | 27.516 | 1.00 | 12.16 |
| ATOM | 2193 | O | GLY | 1102 | 5.348 | 24.410 | 28.437 | 1.00 | 9.75 |
| ATOM | 2194 | N | ALA | 1103 | 4.949 | 25.009 | 26.319 | 1.00 | 12.27 |
| ATOM | 2195 | H | ALA | 1103 | 5.127 | 25.657 | 25.604 | 1.00 | 0.00 |
| ATOM | 2196 | CA | ALA | 1103 | 4.143 | 23.796 | 26.066 | 1.00 | 10.85 |
| ATOM | 2197 | CB | ALA | 1103 | 3.277 | 23.884 | 24.815 | 1.00 | 10.06 |
| ATOM | 2198 | C | ALA | 1103 | 5.024 | 22.580 | 25.839 | 1.00 | 13.18 |
| ATOM | 2199 | O | ALA | 1103 | 6.220 | 22.707 | 25.565 | 1.00 | 13.47 |
| ATOM | 2200 | N | SER | 1104 | 4.467 | 21.373 | 25.983 | 1.00 | 11.96 |
| ATOM | 2201 | H | SER | 1104 | 3.501 | 21.357 | 26.148 | 1.00 | 0.00 |
| ATOM | 2202 | CA | SER | 1104 | 5.204 | 20.090 | 25.772 | 1.00 | 11.74 |
| ATOM | 2203 | CB | SER | 1104 | 4.404 | 18.884 | 26.408 | 1.00 | 12.12 |
| ATOM | 2204 | OG | SER | 1104 | 5.227 | 17.708 | 26.447 | 1.00 | 14.42 |
| ATOM | 2205 | HG | SER | 1104 | 4.649 | 16.936 | 26.552 | 1.00 | 0.00 |
| ATOM | 2206 | C | SER | 1104 | 5.395 | 19.791 | 24.283 | 1.00 | 11.24 |
| ATOM | 2207 | O | SER | 1104 | 4.414 | 19.744 | 23.506 | 1.00 | 11.36 |
| ATOM | 2208 | N | PRO | 1105 | 6.635 | 19.545 | 23.827 | 1.00 | 10.59 |
| ATOM | 2209 | CD | PRO | 1105 | 7.884 | 19.578 | 24.580 | 1.00 | 7.60 |
| ATOM | 2210 | CA | PRO | 1105 | 6.827 | 19.264 | 22.383 | 1.00 | 9.91 |
| ATOM | 2211 | CB | PRO | 1105 | 8.339 | 19.028 | 22.279 | 1.00 | 11.10 |
| ATOM | 2212 | CG | PRO | 1105 | 8.855 | 19.869 | 23.442 | 1.00 | 9.71 |
| ATOM | 2213 | C | PRO | 1105 | 5.968 | 18.113 | 21.810 | 1.00 | 11.75 |
| ATOM | 2214 | O | PRO | 1105 | 5.536 | 17.204 | 22.516 | 1.00 | 12.95 |
| ATOM | 2215 | N | TYR | 1106 | 5.698 | 18.067 | 20.504 | 1.00 | 11.82 |
| ATOM | 2216 | H | TYR | 1106 | 6.057 | 18.807 | 19.984 | 1.00 | 0.00 |
| ATOM | 2217 | CA | TYR | 1106 | 4.864 | 17.022 | 19.803 | 1.00 | 12.44 |
| ATOM | 2218 | CB | TYR | 1106 | 5.602 | 15.643 | 19.666 | 1.00 | 11.08 |
| ATOM | 2219 | CG | TYR | 1106 | 6.821 | 15.859 | 18.861 | 1.00 | 11.02 |
| ATOM | 2220 | CD1 | TYR | 1106 | 8.028 | 16.078 | 19.516 | 1.00 | 9.52 |
| ATOM | 2221 | CE1 | TYR | 1106 | 9.171 | 16.334 | 18.773 | 1.00 | 9.20 |
| ATOM | 2222 | CD2 | TYR | 1106 | 6.748 | 15.885 | 17.459 | 1.00 | 11.79 |
| ATOM | 2223 | CE2 | TYR | 1106 | 7.904 | 16.144 | 16.712 | 1.00 | 8.37 |
| ATOM | 2224 | CZ | TYR | 1106 | 9.111 | 16.374 | 17.371 | 1.00 | 9.25 |
| ATOM | 2225 | OH | TYR | 1106 | 10.258 | 16.708 | 16.623 | 1.00 | 12.44 |
| ATOM | 2226 | HH | TYR | 1106 | 10.298 | 16.112 | 15.857 | 1.00 | 0.00 |
| ATOM | 2227 | C | TYR | 1106 | 3.522 | 16.768 | 20.488 | 1.00 | 14.39 |
| ATOM | 2228 | O | TYR | 1106 | 3.207 | 15.656 | 20.921 | 1.00 | 13.26 |
| ATOM | 2229 | N | PRO | 1107 | 2.665 | 17.818 | 20.616 | 1.00 | 16.19 |
| ATOM | 2230 | CD | PRO | 1107 | 2.787 | 19.216 | 20.101 | 1.00 | 14.47 |
| ATOM | 2231 | CA | PRO | 1107 | 1.373 | 17.604 | 21.290 | 1.00 | 16.73 |
| ATOM | 2232 | CB | PRO | 1107 | 0.665 | 18.932 | 21.132 | 1.00 | 14.38 |
| ATOM | 2233 | CG | PRO | 1107 | 1.312 | 19.624 | 19.930 | 1.00 | 13.95 |
| ATOM | 2234 | C | PRO | 1107 | 0.578 | 16.426 | 20.691 | 1.00 | 19.50 |
| ATOM | 2235 | O | PRO | 1107 | 0.501 | 16.260 | 19.475 | 1.00 | 19.62 |
| ATOM | 2236 | N | GLY | 1108 | −0.046 | 15.573 | 21.483 | 1.00 | 23.51 |
| ATOM | 2237 | H | GLY | 1108 | 0.031 | 15.669 | 22.452 | 1.00 | 0.00 |
| ATOM | 2238 | CA | GLY | 1108 | −0.842 | 14.440 | 20.894 | 1.00 | 29.10 |
| ATOM | 2239 | C | GLY | 1108 | −0.102 | 13.160 | 20.438 | 1.00 | 31.85 |
| ATOM | 2240 | O | GLY | 1108 | −0.678 | 12.081 | 20.383 | 1.00 | 37.36 |
| ATOM | 2241 | N | VAL | 1109 | 1.183 | 13.236 | 20.110 | 1.00 | 32.04 |
| ATOM | 2242 | H | VAL | 1109 | 1.586 | 14.131 | 20.143 | 1.00 | 0.00 |
| ATOM | 2243 | CA | VAL | 1109 | 1.969 | 12.095 | 19.644 | 1.00 | 28.31 |
| ATOM | 2244 | CB | VAL | 1109 | 3.155 | 12.659 | 18.850 | 1.00 | 28.43 |
| ATOM | 2245 | CG1 | VAL | 1109 | 3.876 | 11.519 | 18.123 | 1.00 | 30.74 |
| ATOM | 2246 | CG2 | VAL | 1109 | 2.679 | 13.646 | 17.780 | 1.00 | 25.68 |
| ATOM | 2247 | C | VAL | 1109 | 2.467 | 11.153 | 20.716 | 1.00 | 28.13 |
| ATOM | 2248 | O | VAL | 1109 | 2.965 | 11.598 | 21.741 | 1.00 | 27.78 |
| ATOM | 2249 | N | LYS | 1110 | 2.259 | 9.824 | 20.607 | 1.00 | 28.37 |
| ATOM | 2250 | H | LYS | 1110 | 1.682 | 9.508 | 19.885 | 1.00 | 0.00 |
| ATOM | 2251 | CA | LYS | 1110 | 2.867 | 8.874 | 21.614 | 1.00 | 26.23 |
| ATOM | 2252 | CB | LYS | 1110 | 2.287 | 7.395 | 21.594 | 1.00 | 25.42 |
| ATOM | 2253 | C | LYS | 1110 | 4.329 | 8.771 | 21.145 | 1.00 | 25.25 |
| ATOM | 2254 | O | LYS | 1110 | 4.556 | 8.664 | 19.924 | 1.00 | 26.31 |
| ATOM | 2255 | N | ILE | 1111 | 5.333 | 8.870 | 22.017 | 1.00 | 24.38 |
| ATOM | 2256 | H | ILE | 1111 | 5.095 | 8.999 | 22.962 | 1.00 | 0.00 |
| ATOM | 2257 | CA | ILE | 1111 | 6.765 | 8.789 | 21.606 | 1.00 | 25.28 |
| ATOM | 2258 | CB | ILE | 1111 | 7.512 | 9.665 | 22.621 | 1.00 | 22.13 |
| ATOM | 2259 | CG2 | ILE | 1111 | 9.045 | 9.559 | 22.420 | 1.00 | 21.70 |
| ATOM | 2260 | CG1 | ILE | 1111 | 6.927 | 11.139 | 22.483 | 1.00 | 21.23 |
| ATOM | 2261 | CD1 | ILE | 1111 | 6.818 | 11.920 | 21.179 | 1.00 | 13.98 |
| ATOM | 2262 | C | ILE | 1111 | 7.236 | 7.325 | 21.514 | 1.00 | 27.88 |
| ATOM | 2263 | O | ILE | 1111 | 7.717 | 6.680 | 22.451 | 1.00 | 29.94 |
| ATOM | 2264 | N | ASP | 1112 | 6.945 | 6.785 | 20.326 | 1.00 | 30.41 |
| ATOM | 2265 | H | ASP | 1112 | 6.439 | 7.385 | 19.741 | 1.00 | 0.00 |
| ATOM | 2266 | CA | ASP | 1112 | 7.155 | 5.384 | 19.884 | 1.00 | 31.17 |
| ATOM | 2267 | CB | ASP | 1112 | 5.740 | 4.754 | 19.553 | 1.00 | 34.06 |
| ATOM | 2268 | CG | ASP | 1112 | 4.881 | 5.381 | 18.414 | 1.00 | 38.82 |
| ATOM | 2269 | OD1 | ASP | 1112 | 3.805 | 4.851 | 18.109 | 1.00 | 43.82 |
| ATOM | 2270 | OD2 | ASP | 1112 | 5.213 | 6.410 | 17.836 | 1.00 | 41.73 |
| ATOM | 2271 | C | ASP | 1112 | 8.059 | 5.172 | 18.706 | 1.00 | 31.49 |
| ATOM | 2272 | O | ASP | 1112 | 8.722 | 6.065 | 18.211 | 1.00 | 30.29 |
| ATOM | 2273 | N | GLU | 1113 | 8.070 | 3.949 | 18.179 | 1.00 | 33.84 |
| ATOM | 2274 | H | GLU | 1113 | 7.587 | 3.251 | 18.662 | 1.00 | 0.00 |
| ATOM | 2275 | CA | GLU | 1113 | 8.880 | 3.576 | 16.977 | 1.00 | 35.18 |
| ATOM | 2276 | CB | GLU | 1113 | 8.775 | 2.056 | 16.628 | 1.00 | 36.26 |
| ATOM | 2277 | C | GLU | 1113 | 8.358 | 4.348 | 15.766 | 1.00 | 35.54 |
| ATOM | 2278 | O | GLU | 1113 | 9.078 | 4.776 | 14.856 | 1.00 | 36.48 |
| ATOM | 2279 | N | GLU | 1114 | 7.040 | 4.516 | 15.722 | 1.00 | 35.87 |
| ATOM | 2280 | H | GLU | 1114 | 6.482 | 4.002 | 16.336 | 1.00 | 0.00 |
| ATOM | 2281 | CA | GLU | 1114 | 6.411 | 5.292 | 14.640 | 1.00 | 35.41 |
| ATOM | 2282 | CB | GLU | 1114 | 4.875 | 5.213 | 14.747 | 1.00 | 41.38 |
| ATOM | 2283 | CG | GLU | 1114 | 4.458 | 3.776 | 14.388 | 1.00 | 51.35 |
| ATOM | 2284 | CD | GLU | 1114 | 4.909 | 3.458 | 12.944 | 1.00 | 56.44 |
| ATOM | 2285 | OE1 | GLU | 1114 | 6.030 | 2.921 | 12.757 | 1.00 | 58.09 |
| ATOM | 2286 | OE2 | GLU | 1114 | 4.111 | 3.764 | 12.034 | 1.00 | 59.11 |
| ATOM | 2287 | C | GLU | 1114 | 6.841 | 6.721 | 14.702 | 1.00 | 30.75 |
| ATOM | 2288 | O | GLU | 1114 | 7.162 | 7.328 | 13.696 | 1.00 | 30.84 |
| ATOM | 2289 | N | PHE | 1115 | 6.902 | 7.245 | 15.910 | 1.00 | 26.70 |
| ATOM | 2290 | H | PHE | 1115 | 6.809 | 6.649 | 16.672 | 1.00 | 0.00 |
| ATOM | 2291 | CA | PHE | 1115 | 7.304 | 8.638 | 16.117 | 1.00 | 24.75 |
| ATOM | 2292 | CB | PHE | 1115 | 7.287 | 8.903 | 17.626 | 1.00 | 21.06 |
| ATOM | 2293 | CG | PHE | 1115 | 8.057 | 10.107 | 17.885 | 1.00 | 20.16 |
| ATOM | 2294 | CD1 | PHE | 1115 | 9.337 | 9.975 | 18.448 | 1.00 | 17.72 |
| ATOM | 2295 | CD2 | PHE | 1115 | 7.529 | 11.357 | 17.526 | 1.00 | 18.90 |
| ATOM | 2296 | CE1 | PHE | 1115 | 10.114 | 11.122 | 18.651 | 1.00 | 20.23 |
| ATOM | 2297 | CE2 | PHE | 1115 | 8.310 | 12.504 | 17.728 | 1.00 | 17.75 |
| ATOM | 2298 | CZ | PHE | 1115 | 9.604 | 12.398 | 18.286 | 1.00 | 18.88 |
| ATOM | 2299 | C | PHE | 1115 | 8.687 | 8.809 | 15.488 | 1.00 | 24.04 |
| ATOM | 2300 | O | PHE | 1115 | 8.923 | 9.656 | 14.619 | 1.00 | 20.53 |
| ATOM | 2301 | N | CYS | 1116 | 9.606 | 7.917 | 15.882 | 1.00 | 24.94 |
| ATOM | 2302 | H | CYS | 1116 | 9.387 | 7.294 | 16.606 | 1.00 | 0.00 |
| ATOM | 2303 | CA | CYS | 1116 | 10.978 | 7.952 | 15.320 | 1.00 | 25.77 |
| ATOM | 2304 | CB | CYS | 1116 | 11.873 | 6.944 | 16.088 | 1.00 | 25.14 |
| ATOM | 2305 | SG | CYS | 1116 | 12.087 | 7.227 | 17.838 | 1.00 | 25.53 |
| ATOM | 2306 | C | CYS | 1116 | 11.009 | 7.649 | 13.792 | 1.00 | 24.95 |
| ATOM | 2307 | O | CYS | 1116 | 11.791 | 8.236 | 13.049 | 1.00 | 26.91 |
| ATOM | 2308 | N | ARG | 1117 | 10.185 | 6.781 | 13.254 | 1.00 | 24.68 |
| ATOM | 2309 | H | ARG | 1117 | 9.548 | 6.312 | 13.825 | 1.00 | 0.00 |
| ATOM | 2310 | CA | ARG | 1117 | 10.211 | 6.523 | 11.814 | 1.00 | 27.82 |
| ATOM | 2311 | CB | ARG | 1117 | 9.284 | 5.303 | 11.534 | 1.00 | 32.56 |
| ATOM | 2312 | CG | ARG | 1117 | 8.647 | 5.271 | 10.120 | 1.00 | 39.42 |
| ATOM | 2313 | CD | ARG | 1117 | 7.118 | 4.958 | 10.146 | 1.00 | 42.88 |
| ATOM | 2314 | NE | ARG | 1117 | 6.350 | 5.670 | 9.094 | 1.00 | 47.03 |
| ATOM | 2315 | HE | ARG | 1117 | 6.715 | 5.743 | 8.188 | 1.00 | 0.00 |
| ATOM | 2316 | CZ | ARG | 1117 | 5.116 | 6.172 | 9.331 | 1.00 | 50.95 |
| ATOM | 2317 | NH1 | ARG | 1117 | 4.442 | 6.821 | 8.365 | 1.00 | 52.81 |
| ATOM | 2318 | HH11 | ARG | 1117 | 4.836 | 6.940 | 7.454 | 1.00 | 0.00 |
| ATOM | 2319 | HH12 | ARG | 1117 | 3.534 | 7.180 | 8.581 | 1.00 | 0.00 |
| ATOM | 2320 | NH2 | ARG | 1117 | 4.510 | 6.035 | 10.527 | 1.00 | 53.03 |
| ATOM | 2321 | HH21 | ARG | 1117 | 4.979 | 5.551 | 11.268 | 1.00 | 0.00 |
| ATOM | 2322 | HH22 | ARG | 1117 | 3.598 | 6.414 | 10.688 | 1.00 | 0.00 |
| ATOM | 2323 | C | ARG | 1117 | 9.752 | 7.781 | 11.102 | 1.00 | 27.54 |
| ATOM | 2324 | O | ARG | 1117 | 10.414 | 8.317 | 10.215 | 1.00 | 28.29 |
| ATOM | 2325 | N | ARG | 1118 | 8.625 | 8.347 | 11.512 | 1.00 | 26.52 |
| ATOM | 2326 | H | ARG | 1118 | 8.114 | 7.886 | 12.202 | 1.00 | 0.00 |
| ATOM | 2327 | CA | ARG | 1118 | 8.090 | 9.586 | 10.903 | 1.00 | 25.89 |
| ATOM | 2328 | CB | ARG | 1118 | 6.765 | 9.875 | 11.584 | 1.00 | 28.99 |
| ATOM | 2329 | CG | ARG | 1118 | 5.572 | 9.143 | 10.923 | 1.00 | 35.90 |
| ATOM | 2330 | CD | ARG | 1118 | 4.192 | 9.237 | 11.655 | 1.00 | 43.45 |
| ATOM | 2331 | NE | ARG | 1118 | 4.266 | 10.246 | 12.743 | 1.00 | 51.43 |
| ATOM | 2332 | HE | ARG | 1118 | 4.243 | 11.186 | 12.471 | 1.00 | 0.00 |
| ATOM | 2333 | CZ | ARG | 1118 | 4.328 | 9.999 | 14.092 | 1.00 | 54.13 |
| ATOM | 2334 | NH1 | ARG | 1118 | 4.411 | 11.067 | 14.881 | 1.00 | 56.25 |
| ATOM | 2335 | HH11 | ARG | 1118 | 4.413 | 11.980 | 14.474 | 1.00 | 0.00 |
| ATOM | 2336 | HH12 | ARG | 1118 | 4.457 | 10.953 | 15.873 | 1.00 | 0.00 |
| ATOM | 2337 | NH2 | ARG | 1118 | 4.253 | 8.791 | 14.696 | 1.00 | 54.29 |
| ATOM | 2338 | HH21 | ARG | 1118 | 4.146 | 7.957 | 14.155 | 1.00 | 0.00 |
| ATOM | 2339 | HH22 | ARG | 1118 | 4.307 | 8.729 | 15.695 | 1.00 | 0.00 |
| ATOM | 2340 | C | ARG | 1118 | 9.060 | 10.768 | 11.021 | 1.00 | 24.33 |
| ATOM | 2341 | O | ARG | 1118 | 9.215 | 11.621 | 10.140 | 1.00 | 21.65 |
| ATOM | 2342 | N | LEU | 1119 | 9.769 | 10.844 | 12.135 | 1.00 | 24.38 |
| ATOM | 2343 | H | LEU | 1119 | 9.596 | 10.185 | 12.842 | 1.00 | 0.00 |
| ATOM | 2344 | CA | LEU | 1119 | 10.776 | 11.922 | 12.319 | 1.00 | 26.92 |
| ATOM | 2345 | CB | LEU | 1119 | 11.499 | 11.793 | 13.659 | 1.00 | 26.37 |
| ATOM | 2346 | CG | LEU | 1119 | 12.313 | 13.052 | 13.880 | 1.00 | 28.47 |
| ATOM | 2347 | CD1 | LEU | 1119 | 11.292 | 14.237 | 14.111 | 1.00 | 28.65 |
| ATOM | 2348 | CD2 | LEU | 1119 | 13.418 | 12.730 | 14.903 | 1.00 | 28.41 |
| ATOM | 2349 | C | LEU | 1119 | 11.855 | 11.783 | 11.225 | 1.00 | 28.63 |
| ATOM | 2350 | O | LEU | 1119 | 12.303 | 12.711 | 10.540 | 1.00 | 26.53 |
| ATOM | 2351 | N | LYS | 1120 | 12.321 | 10.537 | 11.070 | 1.00 | 32.80 |
| ATOM | 2352 | H | LYS | 1120 | 11.984 | 9.828 | 11.660 | 1.00 | 0.00 |
| ATOM | 2353 | CA | LYS | 1120 | 13.334 | 10.205 | 10.042 | 1.00 | 35.41 |
| ATOM | 2354 | CB | LYS | 1120 | 13.731 | 8.702 | 10.216 | 1.00 | 37.92 |
| ATOM | 2355 | CG | LYS | 1120 | 15.171 | 8.623 | 9.686 | 1.00 | 42.81 |
| ATOM | 2356 | CD | LYS | 1120 | 15.943 | 7.327 | 9.980 | 1.00 | 46.89 |
| ATOM | 2357 | CE | LYS | 1120 | 17.453 | 7.567 | 9.673 | 1.00 | 49.85 |
| ATOM | 2358 | NZ | LYS | 1120 | 18.322 | 6.519 | 10.248 | 1.00 | 50.53 |
| ATOM | 2359 | HZ1 | LYS | 1120 | 18.005 | 5.592 | 9.897 | 1.00 | 0.00 |
| ATOM | 2360 | HZ2 | LYS | 1120 | 18.232 | 6.537 | 11.285 | 1.00 | 0.00 |
| ATOM | 2361 | HZ3 | LYS | 1120 | 19.313 | 6.677 | 9.980 | 1.00 | 0.00 |
| ATOM | 2362 | C | LYS | 1120 | 12.804 | 10.520 | 8.604 | 1.00 | 34.31 |
| ATOM | 2363 | O | LYS | 1120 | 13.549 | 10.962 | 7.726 | 1.00 | 34.06 |
| ATOM | 2364 | N | GLU | 1121 | 11.519 | 10.335 | 8.307 | 1.00 | 33.69 |
| ATOM | 2365 | H | GLU | 1121 | 10.934 | 9.950 | 8.992 | 1.00 | 0.00 |
| ATOM | 2366 | CA | GLU | 1121 | 11.006 | 10.691 | 6.954 | 1.00 | 34.42 |
| ATOM | 2367 | CB | GLU | 1121 | 9.717 | 9.963 | 6.615 | 1.00 | 39.02 |
| ATOM | 2368 | CG | GLU | 1121 | 9.355 | 8.690 | 7.363 | 1.00 | 44.84 |
| ATOM | 2369 | CD | GLU | 1121 | 7.879 | 8.376 | 7.095 | 1.00 | 50.33 |
| ATOM | 2370 | OE1 | GLU | 1121 | 7.001 | 9.257 | 7.243 | 1.00 | 52.50 |
| ATOM | 2371 | OE2 | GLU | 1121 | 7.637 | 7.216 | 6.746 | 1.00 | 53.15 |
| ATOM | 2372 | C | GLU | 1121 | 10.683 | 12.189 | 6.721 | 1.00 | 31.43 |
| ATOM | 2373 | O | GLU | 1121 | 10.164 | 12.554 | 5.673 | 1.00 | 32.37 |
| ATOM | 2374 | N | GLY | 1122 | 10.901 | 13.115 | 7.670 | 1.00 | 30.06 |
| ATOM | 2375 | H | GLY | 1122 | 11.281 | 12.828 | 8.528 | 1.00 | 0.00 |
| ATOM | 2376 | CA | GLY | 1122 | 10.589 | 14.551 | 7.428 | 1.00 | 25.25 |
| ATOM | 2377 | C | GLY | 1122 | 9.477 | 15.135 | 8.317 | 1.00 | 24.23 |
| ATOM | 2378 | O | GLY | 1122 | 9.157 | 16.314 | 8.213 | 1.00 | 23.78 |
| ATOM | 2379 | N | THR | 1123 | 8.799 | 14.353 | 9.162 | 1.00 | 23.61 |
| ATOM | 2380 | H | THR | 1123 | 8.979 | 13.388 | 9.187 | 1.00 | 0.00 |
| ATOM | 2381 | CA | THR | 1123 | 7.766 | 14.947 | 10.033 | 1.00 | 22.23 |
| ATOM | 2382 | CB | THR | 1123 | 6.974 | 13.900 | 10.800 | 1.00 | 26.33 |
| ATOM | 2383 | OG1 | THR | 1123 | 6.414 | 12.996 | 9.845 | 1.00 | 29.98 |
| ATOM | 2384 | HG1 | THR | 1123 | 5.988 | 12.287 | 10.334 | 1.00 | 0.00 |
| ATOM | 2385 | CG2 | THR | 1123 | 5.802 | 14.510 | 11.618 | 1.00 | 27.93 |
| ATOM | 2386 | C | THR | 1123 | 8.372 | 15.870 | 11.083 | 1.00 | 20.93 |
| ATOM | 2387 | O | THR | 1123 | 9.245 | 15.496 | 11.882 | 1.00 | 20.53 |
| ATOM | 2388 | N | ARG | 1124 | 7.804 | 17.081 | 11.125 | 1.00 | 18.85 |
| ATOM | 2389 | H | ARG | 1124 | 7.091 | 17.224 | 10.463 | 1.00 | 0.00 |
| ATOM | 2390 | CA | ARG | 1124 | 8.208 | 18.177 | 12.015 | 1.00 | 16.91 |
| ATOM | 2391 | CB | ARG | 1124 | 8.866 | 19.297 | 11.205 | 1.00 | 13.51 |
| ATOM | 2392 | CG | ARG | 1124 | 10.111 | 18.857 | 10.438 | 1.00 | 12.60 |
| ATOM | 2393 | CD | ARG | 1124 | 11.153 | 18.234 | 11.408 | 1.00 | 13.51 |
| ATOM | 2394 | NE | ARG | 1124 | 12.359 | 17.884 | 10.659 | 1.00 | 14.82 |
| ATOM | 2395 | HE | ARG | 1124 | 12.937 | 18.607 | 10.337 | 1.00 | 0.00 |
| ATOM | 2396 | CZ | ARG | 1124 | 12.679 | 16.636 | 10.339 | 1.00 | 13.97 |
| ATOM | 2397 | NH1 | ARG | 1124 | 11.903 | 15.614 | 10.685 | 1.00 | 15.09 |
| ATOM | 2398 | HH11 | ARG | 1124 | 11.062 | 15.778 | 11.193 | 1.00 | 0.00 |
| ATOM | 2399 | HH12 | ARG | 1124 | 12.157 | 14.680 | 10.427 | 1.00 | 0.00 |
| ATOM | 2400 | NH2 | ARG | 1124 | 13.830 | 16.422 | 9.696 | 1.00 | 12.22 |
| ATOM | 2401 | HH21 | ARG | 1124 | 14.418 | 17.197 | 9.450 | 1.00 | 0.00 |
| ATOM | 2402 | HH22 | ARG | 1124 | 14.099 | 15.489 | 9.450 | 1.00 | 0.00 |
| ATOM | 2403 | C | ARG | 1124 | 7.018 | 18.769 | 12.732 | 1.00 | 17.72 |
| ATOM | 2404 | O | ARG | 1124 | 5.877 | 18.634 | 12.327 | 1.00 | 18.48 |
| ATOM | 2405 | N | MET | 1125 | 7.203 | 19.426 | 13.869 | 1.00 | 17.44 |
| ATOM | 2406 | H | MET | 1125 | 8.086 | 19.748 | 14.121 | 1.00 | 0.00 |
| ATOM | 2407 | CA | MET | 1125 | 6.082 | 20.058 | 14.545 | 1.00 | 14.08 |
| ATOM | 2408 | CB | MET | 1125 | 6.458 | 20.646 | 15.955 | 1.00 | 11.92 |
| ATOM | 2409 | CG | MET | 1125 | 6.715 | 19.548 | 16.931 | 1.00 | 6.55 |
| ATOM | 2410 | SD | MET | 1125 | 6.981 | 20.313 | 18.505 | 1.00 | 10.93 |
| ATOM | 2411 | CE | MET | 1125 | 8.781 | 20.363 | 18.497 | 1.00 | 6.60 |
| ATOM | 2412 | C | MET | 1125 | 5.590 | 21.244 | 13.756 | 1.00 | 14.73 |
| ATOM | 2413 | O | MET | 1125 | 6.306 | 21.868 | 12.977 | 1.00 | 12.91 |
| ATOM | 2414 | N | ARG | 1126 | 4.311 | 21.498 | 13.970 | 1.00 | 18.11 |
| ATOM | 2415 | H | ARG | 1126 | 3.810 | 20.759 | 14.355 | 1.00 | 0.00 |
| ATOM | 2416 | CA | ARG | 1126 | 3.575 | 22.672 | 13.433 | 1.00 | 21.46 |
| ATOM | 2417 | CB | ARG | 1126 | 2.009 | 22.609 | 13.666 | 1.00 | 27.99 |
| ATOM | 2418 | CG | ARG | 1126 | 1.748 | 22.490 | 15.282 | 1.00 | 39.67 |
| ATOM | 2419 | CD | ARG | 1126 | 1.082 | 23.598 | 16.246 | 1.00 | 44.30 |
| ATOM | 2420 | NE | ARG | 1126 | 1.406 | 23.465 | 17.724 | 1.00 | 45.26 |
| ATOM | 2421 | HE | ARG | 1126 | 2.197 | 22.939 | 17.964 | 1.00 | 0.00 |
| ATOM | 2422 | CZ | ARG | 1126 | 0.693 | 24.111 | 18.739 | 1.00 | 48.13 |
| ATOM | 2423 | NH1 | ARG | 1126 | 1.046 | 23.944 | 20.032 | 1.00 | 40.37 |
| ATOM | 2424 | HH11 | ARG | 1126 | 1.828 | 23.365 | 20.256 | 1.00 | 0.00 |
| ATOM | 2425 | HH12 | ARG | 1126 | 0.525 | 24.403 | 20.750 | 1.00 | 0.00 |
| ATOM | 2426 | NH2 | ARG | 1126 | −0.399 | 24.908 | 18.502 | 1.00 | 47.73 |
| ATOM | 2427 | HH21 | ARG | 1126 | −0.725 | 25.062 | 17.566 | 1.00 | 0.00 |
| ATOM | 2428 | HH22 | ARG | 1126 | −0.880 | 25.345 | 19.263 | 1.00 | 0.00 |
| ATOM | 2429 | C | ARG | 1126 | 4.078 | 23.876 | 14.272 | 1.00 | 18.73 |
| ATOM | 2430 | O | ARG | 1126 | 4.569 | 23.765 | 15.415 | 1.00 | 16.59 |
| ATOM | 2431 | N | ALA | 1127 | 3.756 | 25.044 | 13.748 | 1.00 | 16.28 |
| ATOM | 2432 | H | ALA | 1127 | 3.261 | 25.016 | 12.909 | 1.00 | 0.00 |
| ATOM | 2433 | CA | ALA | 1127 | 4.114 | 26.305 | 14.352 | 1.00 | 15.65 |
| ATOM | 2434 | CB | ALA | 1127 | 3.634 | 27.460 | 13.464 | 1.00 | 14.55 |
| ATOM | 2435 | C | ALA | 1127 | 3.501 | 26.469 | 15.701 | 1.00 | 16.26 |
| ATOM | 2436 | O | ALA | 1127 | 2.282 | 26.263 | 15.831 | 1.00 | 18.84 |
| ATOM | 2437 | N | PRO | 1128 | 4.252 | 26.895 | 16.708 | 1.00 | 14.96 |
| ATOM | 2438 | CD | PRO | 1128 | 5.715 | 27.060 | 16.794 | 1.00 | 12.66 |
| ATOM | 2439 | CA | PRO | 1128 | 3.593 | 27.109 | 18.019 | 1.00 | 14.67 |
| ATOM | 2440 | CB | PRO | 1128 | 4.808 | 27.180 | 18.976 | 1.00 | 15.68 |
| ATOM | 2441 | CG | PRO | 1128 | 5.849 | 27.877 | 18.090 | 1.00 | 11.30 |
| ATOM | 2442 | C | PRO | 1128 | 2.683 | 28.350 | 18.036 | 1.00 | 13.94 |
| ATOM | 2443 | O | PRO | 1128 | 2.848 | 29.271 | 17.226 | 1.00 | 14.43 |
| ATOM | 2444 | N | ASP | 1129 | 1.822 | 28.527 | 19.022 | 1.00 | 12.40 |
| ATOM | 2445 | H | ASP | 1129 | 1.739 | 27.820 | 19.688 | 1.00 | 0.00 |
| ATOM | 2446 | CA | ASP | 1129 | 0.917 | 29.693 | 19.027 | 1.00 | 13.83 |
| ATOM | 2447 | CB | ASP | 1129 | −0.066 | 29.623 | 20.196 | 1.00 | 11.72 |
| ATOM | 2448 | CG | ASP | 1129 | −0.997 | 28.410 | 20.124 | 1.00 | 15.51 |
| ATOM | 2449 | OD1 | ASP | 1129 | −1.572 | 28.083 | 21.172 | 1.00 | 15.59 |
| ATOM | 2450 | OD2 | ASP | 1129 | −1.170 | 27.800 | 19.062 | 1.00 | 13.79 |
| ATOM | 2451 | C | ASP | 1129 | 1.462 | 31.104 | 19.050 | 1.00 | 13.47 |
| ATOM | 2452 | O | ASP | 1129 | 0.829 | 32.019 | 18.529 | 1.00 | 12.94 |
| ATOM | 2453 | N | TYR | 1130 | 2.624 | 31.334 | 19.632 | 1.00 | 11.61 |
| ATOM | 2454 | H | TYR | 1130 | 3.150 | 30.591 | 19.999 | 1.00 | 0.00 |
| ATOM | 2455 | CA | TYR | 1130 | 3.116 | 32.708 | 19.742 | 1.00 | 10.83 |
| ATOM | 2456 | CB | TYR | 1130 | 3.551 | 32.896 | 21.258 | 1.00 | 7.34 |
| ATOM | 2457 | CG | TYR | 1130 | 2.491 | 32.424 | 22.193 | 1.00 | 11.05 |
| ATOM | 2458 | CD1 | TYR | 1130 | 2.536 | 31.128 | 22.766 | 1.00 | 10.80 |
| ATOM | 2459 | CE1 | TYR | 1130 | 1.486 | 30.661 | 23.592 | 1.00 | 10.57 |
| ATOM | 2460 | CD2 | TYR | 1130 | 1.377 | 33.250 | 22.457 | 1.00 | 10.25 |
| ATOM | 2461 | CE2 | TYR | 1130 | 0.333 | 32.812 | 23.270 | 1.00 | 13.26 |
| ATOM | 2462 | CZ | TYR | 1130 | 0.374 | 31.511 | 23.845 | 1.00 | 14.32 |
| ATOM | 2463 | OH | TYR | 1130 | −0.718 | 31.073 | 24.630 | 1.00 | 16.33 |
| ATOM | 2464 | HH | TYR | 1130 | −0.370 | 30.436 | 25.263 | 1.00 | 0.00 |
| ATOM | 2465 | C | TYR | 1130 | 4.249 | 33.022 | 18.776 | 1.00 | 11.87 |
| ATOM | 2466 | O | TYR | 1130 | 4.990 | 34.000 | 18.928 | 1.00 | 11.86 |
| ATOM | 2467 | N | THR | 1131 | 4.476 | 32.146 | 17.811 | 1.00 | 12.72 |
| ATOM | 2468 | H | THR | 1131 | 3.843 | 31.420 | 17.653 | 1.00 | 0.00 |
| ATOM | 2469 | CA | THR | 1131 | 5.646 | 32.365 | 16.935 | 1.00 | 13.59 |
| ATOM | 2470 | CB | THR | 1131 | 6.072 | 31.014 | 16.195 | 1.00 | 13.38 |
| ATOM | 2471 | OG1 | THR | 1131 | 7.360 | 31.276 | 15.706 | 1.00 | 13.01 |
| ATOM | 2472 | HG1 | THR | 1131 | 7.667 | 30.528 | 15.188 | 1.00 | 0.00 |
| ATOM | 2473 | CG2 | THR | 1131 | 5.273 | 30.564 | 14.946 | 1.00 | 8.91 |
| ATOM | 2474 | C | THR | 1131 | 5.548 | 33.452 | 15.870 | 1.00 | 16.94 |
| ATOM | 2475 | O | THR | 1131 | 4.459 | 33.971 | 15.603 | 1.00 | 16.59 |
| ATOM | 2476 | N | THR | 1132 | 6.696 | 33.866 | 15.333 | 1.00 | 16.17 |
| ATOM | 2477 | H | THR | 1132 | 7.527 | 33.431 | 15.621 | 1.00 | 0.00 |
| ATOM | 2478 | CA | THR | 1132 | 6.688 | 34.798 | 14.213 | 1.00 | 16.72 |
| ATOM | 2479 | CB | THR | 1132 | 7.794 | 35.785 | 14.221 | 1.00 | 16.34 |
| ATOM | 2480 | OG1 | THR | 1132 | 9.013 | 35.048 | 14.265 | 1.00 | 18.17 |
| ATOM | 2481 | HG1 | THR | 1132 | 9.723 | 35.661 | 14.512 | 1.00 | 0.00 |
| ATOM | 2482 | CG2 | THR | 1132 | 7.608 | 36.796 | 15.359 | 1.00 | 18.54 |
| ATOM | 2483 | C | THR | 1132 | 6.984 | 33.900 | 13.034 | 1.00 | 18.15 |
| ATOM | 2484 | O | THR | 1132 | 7.596 | 32.826 | 13.167 | 1.00 | 18.89 |
| ATOM | 2485 | N | PRO | 1133 | 6.640 | 34.312 | 11.819 | 1.00 | 19.89 |
| ATOM | 2486 | CD | PRO | 1133 | 5.873 | 35.542 | 11.459 | 1.00 | 20.39 |
| ATOM | 2487 | CA | PRO | 1133 | 6.896 | 33.444 | 10.620 | 1.00 | 20.71 |
| ATOM | 2488 | CB | PRO | 1133 | 6.425 | 34.300 | 9.433 | 1.00 | 21.56 |
| ATOM | 2489 | CG | PRO | 1133 | 5.308 | 35.183 | 10.063 | 1.00 | 21.64 |
| ATOM | 2490 | C | PRO | 1133 | 8.360 | 33.024 | 10.506 | 1.00 | 19.03 |
| ATOM | 2491 | O | PRO | 1133 | 8.719 | 31.869 | 10.229 | 1.00 | 16.45 |
| ATOM | 2492 | N | GLU | 1134 | 9.240 | 34.012 | 10.747 | 1.00 | 17.06 |
| ATOM | 2493 | H | GLU | 1134 | 8.907 | 34.913 | 10.915 | 1.00 | 0.00 |
| ATOM | 2494 | CA | GLU | 1134 | 10.686 | 33.715 | 10.691 | 1.00 | 18.51 |
| ATOM | 2495 | CB | GLU | 1134 | 11.534 | 34.999 | 10.575 | 1.00 | 19.66 |
| ATOM | 2496 | CG | GLU | 1134 | 11.114 | 36.296 | 11.211 | 1.00 | 27.66 |
| ATOM | 2497 | CD | GLU | 1134 | 9.863 | 36.966 | 10.602 | 1.00 | 29.76 |
| ATOM | 2498 | OE1 | GLU | 1134 | 8.883 | 37.146 | 11.310 | 1.00 | 31.35 |
| ATOM | 2499 | OE2 | GLU | 1134 | 9.868 | 37.334 | 9.442 | 1.00 | 32.48 |
| ATOM | 2500 | C | GLU | 1134 | 11.165 | 32.881 | 11.856 | 1.00 | 16.49 |
| ATOM | 2501 | O | GLU | 1134 | 12.113 | 32.107 | 11.658 | 1.00 | 14.20 |
| ATOM | 2502 | N | MET | 1135 | 10.543 | 32.986 | 13.079 | 1.00 | 15.23 |
| ATOM | 2503 | H | MET | 1135 | 9.809 | 33.615 | 13.230 | 1.00 | 0.00 |
| ATOM | 2504 | CA | MET | 1135 | 10.989 | 32.092 | 14.169 | 1.00 | 13.39 |
| ATOM | 2505 | CB | MET | 1135 | 10.378 | 32.352 | 15.593 | 1.00 | 13.65 |
| ATOM | 2506 | CG | MET | 1135 | 11.065 | 33.589 | 16.185 | 1.00 | 21.35 |
| ATOM | 2507 | SD | MET | 1135 | 12.898 | 33.531 | 16.320 | 1.00 | 26.08 |
| ATOM | 2508 | CE | MET | 1135 | 13.123 | 31.946 | 17.074 | 1.00 | 15.74 |
| ATOM | 2509 | C | MET | 1135 | 10.581 | 30.666 | 13.764 | 1.00 | 11.94 |
| ATOM | 2510 | O | MET | 1135 | 11.381 | 29.750 | 13.983 | 1.00 | 13.63 |
| ATOM | 2511 | N | TYR | 1136 | 9.410 | 30.412 | 13.178 | 1.00 | 8.29 |
| ATOM | 2512 | H | TYR | 1136 | 8.783 | 31.159 | 13.029 | 1.00 | 0.00 |
| ATOM | 2513 | CA | TYR | 1136 | 9.100 | 29.061 | 12.772 | 1.00 | 7.94 |
| ATOM | 2514 | CB | TYR | 1136 | 7.668 | 29.068 | 12.317 | 1.00 | 9.36 |
| ATOM | 2515 | CG | TYR | 1136 | 7.315 | 27.659 | 12.113 | 1.00 | 10.67 |
| ATOM | 2516 | CD1 | TYR | 1136 | 7.522 | 26.642 | 13.081 | 1.00 | 10.95 |
| ATOM | 2517 | CE1 | TYR | 1136 | 7.179 | 25.295 | 12.818 | 1.00 | 10.32 |
| ATOM | 2518 | CD2 | TYR | 1136 | 6.756 | 27.343 | 10.873 | 1.00 | 10.22 |
| ATOM | 2519 | CE2 | TYR | 1136 | 6.419 | 26.011 | 10.619 | 1.00 | 13.47 |
| ATOM | 2520 | CZ | TYR | 1136 | 6.629 | 25.012 | 11.574 | 1.00 | 9.44 |
| ATOM | 2521 | OH | TYR | 1136 | 6.212 | 23.774 | 11.217 | 1.00 | 11.19 |
| ATOM | 2522 | HH | TYR | 1136 | 6.005 | 23.729 | 10.279 | 1.00 | 0.00 |
| ATOM | 2523 | C | TYR | 1136 | 10.069 | 28.567 | 11.662 | 1.00 | 11.63 |
| ATOM | 2524 | O | TYR | 1136 | 10.560 | 27.433 | 11.685 | 1.00 | 12.69 |
| ATOM | 2525 | N | GLN | 1137 | 10.344 | 29.337 | 10.599 | 1.00 | 11.62 |
| ATOM | 2526 | H | GLN | 1137 | 9.829 | 30.160 | 10.482 | 1.00 | 0.00 |
| ATOM | 2527 | CA | GLN | 1137 | 11.352 | 28.873 | 9.620 | 1.00 | 11.20 |
| ATOM | 2528 | CB | GLN | 1137 | 11.533 | 29.959 | 8.554 | 1.00 | 11.85 |
| ATOM | 2529 | CG | GLN | 1137 | 12.535 | 29.476 | 7.432 | 1.00 | 16.41 |
| ATOM | 2530 | CD | GLN | 1137 | 12.029 | 28.200 | 6.742 | 1.00 | 17.63 |
| ATOM | 2531 | OE1 | GLN | 1137 | 10.914 | 28.141 | 6.204 | 1.00 | 16.74 |
| ATOM | 2532 | NE2 | GLN | 1137 | 12.818 | 27.129 | 6.768 | 1.00 | 14.60 |
| ATOM | 2533 | HE21 | GLN | 1137 | 13.682 | 27.153 | 7.211 | 1.00 | 0.00 |
| ATOM | 2534 | HE22 | GLN | 1137 | 12.503 | 26.320 | 6.325 | 1.00 | 0.00 |
| ATOM | 2535 | C | GLN | 1137 | 12.742 | 28.514 | 10.263 | 1.00 | 10.70 |
| ATOM | 2536 | O | GLN | 1137 | 13.488 | 27.655 | 9.811 | 1.00 | 11.23 |
| ATOM | 2537 | N | THR | 1138 | 13.160 | 29.223 | 11.279 | 1.00 | 10.73 |
| ATOM | 2538 | H | THR | 1138 | 12.711 | 30.080 | 11.430 | 1.00 | 0.00 |
| ATOM | 2539 | CA | THR | 1138 | 14.423 | 28.970 | 11.991 | 1.00 | 12.36 |
| ATOM | 2540 | CB | THR | 1138 | 14.611 | 30.172 | 13.003 | 1.00 | 13.87 |
| ATOM | 2541 | OG1 | THR | 1138 | 14.798 | 31.399 | 12.255 | 1.00 | 13.22 |
| ATOM | 2542 | HG1 | THR | 1138 | 14.185 | 32.065 | 12.593 | 1.00 | 0.00 |
| ATOM | 2543 | CG2 | THR | 1138 | 15.837 | 29.981 | 13.919 | 1.00 | 12.91 |
| ATOM | 2544 | C | THR | 1138 | 14.441 | 27.576 | 12.685 | 1.00 | 13.74 |
| ATOM | 2545 | O | THR | 1138 | 15.450 | 26.864 | 12.656 | 1.00 | 14.41 |
| ATOM | 2546 | N | MET | 1139 | 13.317 | 27.186 | 13.327 | 1.00 | 12.09 |
| ATOM | 2547 | H | MET | 1139 | 12.610 | 27.867 | 13.380 | 1.00 | 0.00 |
| ATOM | 2548 | CA | MET | 1139 | 13.049 | 25.895 | 13.999 | 1.00 | 9.92 |
| ATOM | 2549 | CB | MET | 1139 | 11.598 | 25.843 | 14.510 | 1.00 | 8.95 |
| ATOM | 2550 | CG | MET | 1139 | 11.288 | 26.816 | 15.683 | 1.00 | 11.71 |
| ATOM | 2551 | SD | MET | 1139 | 9.570 | 26.789 | 16.208 | 1.00 | 12.42 |
| ATOM | 2552 | CE | MET | 1139 | 9.378 | 28.445 | 16.808 | 1.00 | 6.39 |
| ATOM | 2553 | C | MET | 1139 | 13.184 | 24.868 | 12.916 | 1.00 | 10.48 |
| ATOM | 2554 | O | MET | 1139 | 13.939 | 23.934 | 12.995 | 1.00 | 11.60 |
| ATOM | 2555 | N | LEU | 1140 | 12.429 | 25.015 | 11.838 | 1.00 | 11.61 |
| ATOM | 2556 | H | LEU | 1140 | 11.756 | 25.730 | 11.841 | 1.00 | 0.00 |
| ATOM | 2557 | CA | LEU | 1140 | 12.520 | 24.102 | 10.670 | 1.00 | 11.52 |
| ATOM | 2558 | CB | LEU | 1140 | 11.640 | 24.629 | 9.516 | 1.00 | 9.13 |
| ATOM | 2559 | CG | LEU | 1140 | 10.152 | 24.631 | 9.713 | 1.00 | 11.56 |
| ATOM | 2560 | CD1 | LEU | 1140 | 9.505 | 25.044 | 8.350 | 1.00 | 11.55 |
| ATOM | 2561 | CD2 | LEU | 1140 | 9.663 | 23.234 | 10.190 | 1.00 | 8.44 |
| ATOM | 2562 | C | LEU | 1140 | 13.978 | 24.023 | 10.170 | 1.00 | 12.08 |
| ATOM | 2563 | O | LEU | 1140 | 14.449 | 22.947 | 9.814 | 1.00 | 14.18 |
| ATOM | 2564 | N | ASP | 1141 | 14.719 | 25.140 | 10.096 | 1.00 | 12.35 |
| ATOM | 2565 | H | ASP | 1141 | 14.271 | 25.995 | 10.248 | 1.00 | 0.00 |
| ATOM | 2566 | CA | ASP | 1141 | 16.136 | 25.136 | 9.671 | 1.00 | 12.11 |
| ATOM | 2567 | CB | ASP | 1141 | 16.783 | 26.574 | 9.759 | 1.00 | 16.14 |
| ATOM | 2568 | CG | ASP | 1141 | 16.311 | 27.576 | 8.684 | 1.00 | 18.95 |
| ATOM | 2569 | OD1 | ASP | 1141 | 16.555 | 28.751 | 8.850 | 1.00 | 19.98 |
| ATOM | 2570 | OD2 | ASP | 1141 | 15.710 | 27.209 | 7.681 | 1.00 | 21.61 |
| ATOM | 2571 | C | ASP | 1141 | 16.929 | 24.218 | 10.567 | 1.00 | 12.00 |
| ATOM | 2572 | O | ASP | 1141 | 17.741 | 23.389 | 10.136 | 1.00 | 10.77 |
| ATOM | 2573 | N | CYS | 1142 | 16.723 | 24.405 | 11.880 | 1.00 | 9.89 |
| ATOM | 2574 | H | CYS | 1142 | 16.113 | 25.128 | 12.146 | 1.00 | 0.00 |
| ATOM | 2575 | CA | CYS | 1142 | 17.402 | 23.596 | 12.931 | 1.00 | 9.14 |
| ATOM | 2576 | CB | CYS | 1142 | 17.077 | 24.052 | 14.410 | 1.00 | 11.06 |
| ATOM | 2577 | SG | CYS | 1142 | 17.594 | 25.770 | 14.791 | 1.00 | 12.04 |
| ATOM | 2578 | C | CYS | 1142 | 17.063 | 22.123 | 12.884 | 1.00 | 10.28 |
| ATOM | 2579 | O | CYS | 1142 | 17.819 | 21.269 | 13.377 | 1.00 | 7.29 |
| ATOM | 2580 | N | TRP | 1143 | 15.858 | 21.826 | 12.340 | 1.00 | 10.05 |
| ATOM | 2581 | H | TRP | 1143 | 15.253 | 22.558 | 12.095 | 1.00 | 0.00 |
| ATOM | 2582 | CA | TRP | 1143 | 15.415 | 20.431 | 12.233 | 1.00 | 9.86 |
| ATOM | 2583 | CB | TRP | 1143 | 13.918 | 20.422 | 12.540 | 1.00 | 8.26 |
| ATOM | 2584 | CG | TRP | 1143 | 13.549 | 20.873 | 13.909 | 1.00 | 9.79 |
| ATOM | 2585 | CD2 | TRP | 1143 | 12.289 | 21.409 | 14.268 | 1.00 | 10.88 |
| ATOM | 2586 | CE2 | TRP | 1143 | 12.307 | 21.612 | 15.682 | 1.00 | 9.03 |
| ATOM | 2587 | CE3 | TRP | 1143 | 11.125 | 21.734 | 13.497 | 1.00 | 12.36 |
| ATOM | 2588 | CD1 | TRP | 1143 | 14.255 | 20.780 | 15.127 | 1.00 | 10.04 |
| ATOM | 2589 | NE1 | TRP | 1143 | 13.503 | 21.225 | 16.193 | 1.00 | 9.01 |
| ATOM | 2590 | HE1 | TRP | 1143 | 13.751 | 21.151 | 17.139 | 1.00 | 0.00 |
| ATOM | 2591 | CZ2 | TRP | 1143 | 11.158 | 22.152 | 16.268 | 1.00 | 9.18 |
| ATOM | 2592 | CZ3 | TRP | 1143 | 9.959 | 22.272 | 14.105 | 1.00 | 10.47 |
| ATOM | 2593 | CH2 | TRP | 1143 | 10.008 | 22.482 | 15.491 | 1.00 | 7.42 |
| ATOM | 2594 | C | TRP | 1143 | 15.735 | 19.788 | 10.869 | 1.00 | 13.41 |
| ATOM | 2595 | O | TRP | 1143 | 15.097 | 18.916 | 10.282 | 1.00 | 13.88 |
| ATOM | 2596 | N | HIS | 1144 | 16.755 | 20.319 | 10.244 | 1.00 | 14.53 |
| ATOM | 2597 | H | HIS | 1144 | 17.160 | 21.120 | 10.622 | 1.00 | 0.00 |
| ATOM | 2598 | CA | HIS | 1144 | 17.233 | 19.814 | 8.986 | 1.00 | 15.86 |
| ATOM | 2599 | C | HIS | 1144 | 17.697 | 18.375 | 9.218 | 1.00 | 15.88 |
| ATOM | 2600 | O | HIS | 1144 | 18.496 | 18.108 | 10.118 | 1.00 | 16.45 |
| ATOM | 2601 | CB | HIS | 1144 | 18.392 | 20.691 | 8.566 | 1.00 | 20.10 |
| ATOM | 2602 | CG | HIS | 1144 | 18.646 | 20.358 | 7.199 | 1.00 | 22.59 |
| ATOM | 2603 | ND1 | HIS | 1144 | 18.033 | 20.945 | 6.157 | 1.00 | 26.27 |
| ATOM | 2604 | HD1 | HIS | 1144 | 17.385 | 21.685 | 6.214 | 1.00 | 0.00 |
| ATOM | 2605 | CD2 | HIS | 1144 | 19.409 | 19.301 | 6.750 | 1.00 | 23.42 |
| ATOM | 2606 | NE2 | HIS | 1144 | 19.259 | 19.230 | 5.419 | 1.00 | 25.41 |
| ATOM | 2607 | CE1 | HIS | 1144 | 18.416 | 20.241 | 5.031 | 1.00 | 24.96 |
| ATOM | 2608 | N | GLY | 1145 | 17.246 | 17.392 | 8.413 | 1.00 | 16.61 |
| ATOM | 2609 | H | GLY | 1145 | 16.441 | 17.623 | 7.917 | 1.00 | 0.00 |
| ATOM | 2610 | CA | GLY | 1145 | 17.676 | 15.964 | 8.534 | 1.00 | 12.99 |
| ATOM | 2611 | C | GLY | 1145 | 19.215 | 15.875 | 8.544 | 1.00 | 15.09 |
| ATOM | 2612 | O | GLY | 1145 | 19.832 | 15.192 | 9.344 | 1.00 | 16.85 |
| ATOM | 2613 | N | GLU | 1146 | 19.901 | 16.561 | 7.641 | 1.00 | 15.41 |
| ATOM | 2614 | H | GLU | 1146 | 19.360 | 16.986 | 6.954 | 1.00 | 0.00 |
| ATOM | 2615 | CA | GLU | 1146 | 21.365 | 16.634 | 7.539 | 1.00 | 16.58 |
| ATOM | 2616 | CB | GLU | 1146 | 21.604 | 16.961 | 6.052 | 1.00 | 22.39 |
| ATOM | 2617 | CG | GLU | 1146 | 23.035 | 16.995 | 5.598 | 1.00 | 31.26 |
| ATOM | 2618 | CD | GLU | 1146 | 23.726 | 15.689 | 5.954 | 1.00 | 38.03 |
| ATOM | 2619 | OE1 | GLU | 1146 | 23.307 | 14.626 | 5.453 | 1.00 | 40.59 |
| ATOM | 2620 | OE2 | GLU | 1146 | 24.692 | 15.781 | 6.735 | 1.00 | 40.52 |
| ATOM | 2621 | C | GLU | 1146 | 22.016 | 17.629 | 8.530 | 1.00 | 13.41 |
| ATOM | 2622 | O | GLU | 1146 | 21.990 | 18.857 | 8.381 | 1.00 | 12.91 |
| ATOM | 2623 | N | PRO | 1147 | 22.687 | 17.102 | 9.549 | 1.00 | 12.44 |
| ATOM | 2624 | CD | PRO | 1147 | 22.895 | 15.673 | 9.777 | 1.00 | 11.68 |
| ATOM | 2625 | CA | PRO | 1147 | 23.378 | 17.844 | 10.592 | 1.00 | 14.05 |
| ATOM | 2626 | CB | PRO | 1147 | 24.330 | 16.886 | 11.334 | 1.00 | 12.02 |
| ATOM | 2627 | CG | PRO | 1147 | 23.625 | 15.521 | 11.125 | 1.00 | 11.74 |
| ATOM | 2628 | C | PRO | 1147 | 24.169 | 18.983 | 10.049 | 1.00 | 17.32 |
| ATOM | 2629 | O | PRO | 1147 | 24.142 | 20.089 | 10.584 | 1.00 | 17.23 |
| ATOM | 2630 | N | SER | 1148 | 24.921 | 18.753 | 8.955 | 1.00 | 18.62 |
| ATOM | 2631 | H | SER | 1148 | 24.933 | 17.867 | 8.540 | 1.00 | 0.00 |
| ATOM | 2632 | CA | SER | 1148 | 25.756 | 19.835 | 8.389 | 1.00 | 17.44 |
| ATOM | 2633 | CB | SER | 1148 | 26.761 | 19.390 | 7.332 | 1.00 | 18.22 |
| ATOM | 2634 | OG | SER | 1148 | 26.042 | 18.748 | 6.278 | 1.00 | 20.41 |
| ATOM | 2635 | HG | SER | 1148 | 26.465 | 18.973 | 5.430 | 1.00 | 0.00 |
| ATOM | 2636 | C | SER | 1148 | 24.958 | 20.863 | 7.732 | 1.00 | 16.15 |
| ATOM | 2637 | O | SER | 1148 | 25.505 | 21.917 | 7.491 | 1.00 | 18.02 |
| ATOM | 2638 | N | CLN | 1149 | 23.732 | 20.586 | 7.357 | 1.00 | 15.16 |
| ATOM | 2639 | H | GLN | 1149 | 23.418 | 19.667 | 7.405 | 1.00 | 0.00 |
| ATOM | 2640 | CA | GLN | 1149 | 22.891 | 21.615 | 6.729 | 1.00 | 15.44 |
| ATOM | 2641 | CB | GLN | 1149 | 22.006 | 20.925 | 5.725 | 1.00 | 19.01 |
| ATOM | 2642 | CG | GLN | 1149 | 22.759 | 20.628 | 4.391 | 1.00 | 23.66 |
| ATOM | 2643 | CD | GLN | 1149 | 22.885 | 21.905 | 3.528 | 1.00 | 29.98 |
| ATOM | 2644 | OE1 | GLN | 1149 | 21.961 | 22.400 | 2.876 | 1.00 | 32.54 |
| ATOM | 2645 | NE2 | GLN | 1149 | 23.993 | 22.615 | 3.547 | 1.00 | 34.01 |
| ATOM | 2646 | HE21 | GLN | 1149 | 24.788 | 22.431 | 4.066 | 1.00 | 0.00 |
| ATOM | 2647 | HE22 | GLN | 1149 | 23.947 | 23.404 | 2.963 | 1.00 | 0.00 |
| ATOM | 2648 | C | GLN | 1149 | 22.100 | 22.442 | 7.692 | 1.00 | 14.66 |
| ATOM | 2649 | O | GLN | 1149 | 21.415 | 23.404 | 7.337 | 1.00 | 12.66 |
| ATOM | 2650 | N | ARG | 1150 | 22.050 | 21.997 | 8.948 | 1.00 | 15.39 |
| ATOM | 2651 | H | ARG | 1150 | 22.424 | 21.122 | 9.186 | 1.00 | 0.00 |
| ATOM | 2652 | CA | ARG | 1150 | 21.412 | 22.849 | 10.014 | 1.00 | 13.76 |
| ATOM | 2653 | CB | ARG | 1150 | 21.304 | 22.119 | 11.369 | 1.00 | 10.68 |
| ATOM | 2654 | CG | ARG | 1150 | 20.487 | 20.870 | 11.275 | 1.00 | 6.04 |
| ATOM | 2655 | CD | ARG | 1150 | 20.662 | 20.046 | 12.523 | 1.00 | 10.03 |
| ATOM | 2656 | NE | ARG | 1150 | 20.063 | 18.756 | 12.177 | 1.00 | 10.05 |
| ATOM | 2657 | HE | ARG | 1150 | 19.274 | 18.802 | 11.626 | 1.00 | 0.00 |
| ATOM | 2658 | CZ | ARG | 1150 | 20.400 | 17.611 | 12.748 | 1.00 | 8.82 |
| ATOM | 2659 | NH1 | ARG | 1150 | 21.302 | 17.584 | 13.744 | 1.00 | 6.92 |
| ATOM | 2660 | HH11 | ARG | 1150 | 21.752 | 18.415 | 14.063 | 1.00 | 0.00 |
| ATOM | 2661 | HH12 | ARG | 1150 | 21.537 | 16.706 | 14.152 | 1.00 | 0.00 |
| ATOM | 2662 | NH2 | ARG | 1150 | 19.901 | 16.487 | 12.197 | 1.00 | 6.09 |
| ATOM | 2663 | HH21 | ARG | 1150 | 19.315 | 16.546 | 11.389 | 1.00 | 0.00 |
| ATOM | 2664 | HH22 | ARG | 1150 | 20.098 | 15.588 | 12.589 | 1.00 | 0.00 |
| ATOM | 2665 | C | ARG | 1150 | 22.322 | 24.089 | 10.254 | 1.00 | 14.35 |
| ATOM | 2666 | O | ARG | 1150 | 23.561 | 24.043 | 10.068 | 1.00 | 13.96 |
| ATOM | 2667 | N | PRO | 1151 | 21.790 | 25.263 | 10.656 | 1.00 | 13.41 |
| ATOM | 2668 | CD | PRO | 1151 | 20.359 | 25.620 | 10.792 | 1.00 | 11.75 |
| ATOM | 2669 | CA | PRO | 1151 | 22.716 | 26.415 | 10.974 | 1.00 | 12.90 |
| ATOM | 2670 | CB | PRO | 1151 | 21.739 | 27.599 | 11.290 | 1.00 | 10.89 |
| ATOM | 2671 | CG | PRO | 1151 | 20.400 | 26.930 | 11.646 | 1.00 | 11.67 |
| ATOM | 2672 | C | PRO | 1151 | 23.751 | 26.187 | 12.108 | 1.00 | 12.85 |
| ATOM | 2673 | O | PRO | 1151 | 23.560 | 25.278 | 12.926 | 1.00 | 13.40 |
| ATOM | 2674 | N | THR | 1152 | 24.878 | 26.902 | 12.163 | 1.00 | 11.15 |
| ATOM | 2675 | H | THR | 1152 | 25.145 | 27.467 | 11.423 | 1.00 | 0.00 |
| ATOM | 2676 | CA | THR | 1152 | 25.760 | 26.698 | 13.337 | 1.00 | 10.98 |
| ATOM | 2677 | CB | THR | 1152 | 27.158 | 27.134 | 13.118 | 1.00 | 9.87 |
| ATOM | 2678 | OG1 | THR | 1152 | 27.151 | 28.487 | 12.760 | 1.00 | 10.78 |
| ATOM | 2679 | HG1 | THR | 1152 | 28.078 | 28.726 | 12.644 | 1.00 | 0.00 |
| ATOM | 2680 | CG2 | THR | 1152 | 27.781 | 26.346 | 11.993 | 1.00 | 10.21 |
| ATOM | 2681 | C | THR | 1152 | 25.279 | 27.584 | 14.476 | 1.00 | 9.16 |
| ATOM | 2682 | O | THR | 1152 | 24.421 | 28.422 | 14.208 | 1.00 | 11.34 |
| ATOM | 2683 | N | PHE | 1153 | 25.660 | 27.427 | 15.735 | 1.00 | 7.56 |
| ATOM | 2684 | H | PHE | 1153 | 26.125 | 26.604 | 15.993 | 1.00 | 0.00 |
| ATOM | 2685 | CA | PHE | 1153 | 25.211 | 28.389 | 16.789 | 1.00 | 9.10 |
| ATOM | 2686 | CB | PHE | 1153 | 25.729 | 27.970 | 18.239 | 1.00 | 9.77 |
| ATOM | 2687 | CG | PHE | 1153 | 24.739 | 26.964 | 18.782 | 1.00 | 10.94 |
| ATOM | 2688 | CD1 | PHE | 1153 | 23.381 | 27.326 | 19.022 | 1.00 | 9.31 |
| ATOM | 2689 | CD2 | PHE | 1153 | 25.132 | 25.594 | 18.950 | 1.00 | 9.88 |
| ATOM | 2690 | CE1 | PHE | 1153 | 22.446 | 26.358 | 19.396 | 1.00 | 9.59 |
| ATOM | 2691 | CE2 | PHE | 1153 | 24.188 | 24.624 | 19.324 | 1.00 | 8.24 |
| ATOM | 2692 | CZ | PHE | 1153 | 22.851 | 25.010 | 19.541 | 1.00 | 10.74 |
| ATOM | 2693 | C | PHE | 1153 | 25.680 | 29.827 | 16.460 | 1.00 | 11.29 |
| ATOM | 2694 | O | PHE | 1153 | 25.000 | 30.838 | 16.730 | 1.00 | 11.98 |
| ATOM | 2695 | N | SER | 1154 | 26.829 | 29.997 | 15.790 | 1.00 | 12.07 |
| ATOM | 2696 | H | SER | 1154 | 27.451 | 29.243 | 15.798 | 1.00 | 0.00 |
| ATOM | 2697 | CA | SER | 1154 | 27.257 | 31.382 | 15.383 | 1.00 | 12.79 |
| ATOM | 2698 | CB | SER | 1154 | 28.657 | 31.431 | 14.773 | 1.00 | 12.05 |
| ATOM | 2699 | OG | SER | 1154 | 29.552 | 30.999 | 15.767 | 1.00 | 12.43 |
| ATOM | 2700 | HG | SER | 1154 | 30.424 | 31.394 | 15.623 | 1.00 | 0.00 |
| ATOM | 2701 | C | SER | 1154 | 26.338 | 32.022 | 14.359 | 1.00 | 12.24 |
| ATOM | 2702 | O | SER | 1154 | 26.162 | 33.254 | 14.338 | 1.00 | 11.73 |
| ATOM | 2703 | N | GLU | 1155 | 25.844 | 31.234 | 13.394 | 1.00 | 11.38 |
| ATOM | 2704 | H | GLU | 1155 | 26.062 | 30.278 | 13.405 | 1.00 | 0.00 |
| ATOM | 2705 | CA | GLU | 1155 | 24.869 | 31.760 | 12.387 | 1.00 | 13.34 |
| ATOM | 2706 | CB | GLU | 1155 | 24.658 | 30.709 | 11.297 | 1.00 | 14.17 |
| ATOM | 2707 | CG | GLU | 1155 | 25.965 | 30.441 | 10.526 | 1.00 | 18.38 |
| ATOM | 2708 | CD | GLU | 1155 | 25.928 | 29.256 | 9.566 | 1.00 | 20.81 |
| ATOM | 2709 | OE1 | GLU | 1155 | 26.798 | 29.162 | 8.720 | 1.00 | 21.79 |
| ATOM | 2710 | OE2 | GLU | 1155 | 25.049 | 28.416 | 9.629 | 1.00 | 25.52 |
| ATOM | 2711 | C | GLU | 1155 | 23.521 | 32.081 | 13.079 | 1.00 | 15.20 |
| ATOM | 2712 | O | GLU | 1155 | 22.900 | 33.107 | 12.812 | 1.00 | 16.46 |
| ATOM | 2713 | N | LEU | 1156 | 23.019 | 31.176 | 13.973 | 1.00 | 15.39 |
| ATOM | 2714 | H | LEU | 1156 | 23.473 | 30.306 | 14.053 | 1.00 | 0.00 |
| ATOM | 2715 | CA | LEU | 1156 | 21.782 | 31.403 | 14.762 | 1.00 | 12.64 |
| ATOM | 2716 | CB | LEU | 1156 | 21.558 | 30.195 | 15.717 | 1.00 | 9.65 |
| ATOM | 2717 | CG | LEU | 1156 | 20.973 | 28.969 | 15.009 | 1.00 | 10.18 |
| ATOM | 2718 | CD1 | LEU | 1156 | 21.157 | 27.779 | 15.950 | 1.00 | 4.93 |
| ATOM | 2719 | CD2 | LEU | 1156 | 19.548 | 29.268 | 14.497 | 1.00 | 6.99 |
| ATOM | 2720 | C | LEU | 1156 | 21.938 | 32.717 | 15.586 | 1.00 | 13.69 |
| ATOM | 2721 | O | LEU | 1156 | 21.037 | 33.535 | 15.626 | 1.00 | 13.03 |
| ATOM | 2722 | N | VAL | 1157 | 23.099 | 33.040 | 16.218 | 1.00 | 14.37 |
| ATOM | 2723 | H | VAL | 1157 | 23.776 | 32.335 | 16.232 | 1.00 | 0.00 |
| ATOM | 2724 | CA | VAL | 1157 | 23.335 | 34.285 | 17.012 | 1.00 | 11.68 |
| ATOM | 2725 | CB | VAL | 1157 | 24.794 | 34.172 | 17.589 | 1.00 | 12.78 |
| ATOM | 2726 | CG1 | VAL | 1157 | 25.224 | 35.606 | 18.077 | 1.00 | 10.34 |
| ATOM | 2727 | CG2 | VAL | 1157 | 24.903 | 33.185 | 18.782 | 1.00 | 8.22 |
| ATOM | 2728 | C | VAL | 1157 | 23.136 | 35.565 | 16.141 | 1.00 | 14.56 |
| ATOM | 2729 | O | VAL | 1157 | 22.444 | 36.545 | 16.475 | 1.00 | 13.22 |
| ATOM | 2730 | N | GLU | 1158 | 23.698 | 35.532 | 14.916 | 1.00 | 13.69 |
| ATOM | 2731 | H | GLU | 1158 | 24.179 | 34.719 | 14.664 | 1.00 | 0.00 |
| ATOM | 2732 | CA | GLU | 1158 | 23.581 | 36.667 | 13.955 | 1.00 | 13.62 |
| ATOM | 2733 | CB | GLU | 1158 | 24.457 | 36.424 | 12.670 | 1.00 | 15.22 |
| ATOM | 2734 | CG | GLU | 1158 | 24.428 | 37.532 | 11.587 | 1.00 | 23.30 |
| ATOM | 2735 | CD | GLU | 1158 | 25.089 | 37.106 | 10.241 | 1.00 | 30.69 |
| ATOM | 2736 | OE1 | GLU | 1158 | 24.947 | 35.951 | 9.800 | 1.00 | 34.95 |
| ATOM | 2737 | OE2 | GLU | 1158 | 25.729 | 37.937 | 9.583 | 1.00 | 35.29 |
| ATOM | 2738 | C | GLU | 1158 | 22.160 | 36.803 | 13.553 | 1.00 | 11.52 |
| ATOM | 2739 | O | GLU | 1158 | 21.530 | 37.856 | 13.628 | 1.00 | 13.64 |
| ATOM | 2740 | N | HIS | 1159 | 21.573 | 35.701 | 13.155 | 1.00 | 12.20 |
| ATOM | 2741 | H | HIS | 1159 | 22.104 | 34.881 | 13.087 | 1.00 | 0.00 |
| ATOM | 2742 | CA | HIS | 1159 | 20.166 | 35.691 | 12.713 | 1.00 | 12.06 |
| ATOM | 2743 | C | HIS | 1159 | 19.244 | 36.144 | 13.798 | 1.00 | 10.77 |
| ATOM | 2744 | O | HIS | 1159 | 18.444 | 37.065 | 13.676 | 1.00 | 10.85 |
| ATOM | 2745 | CB | HIS | 1159 | 19.805 | 34.270 | 12.267 | 1.00 | 12.95 |
| ATOM | 2746 | CG | HIS | 1159 | 18.505 | 34.291 | 11.632 | 1.00 | 14.30 |
| ATOM | 2747 | ND1 | HIS | 1159 | 17.510 | 33.405 | 11.936 | 1.00 | 20.58 |
| ATOM | 2748 | HD1 | HIS | 1159 | 17.506 | 32.743 | 12.659 | 1.00 | 0.00 |
| ATOM | 2749 | CD2 | HIS | 1159 | 18.058 | 35.126 | 10.619 | 1.00 | 14.65 |
| ATOM | 2750 | NE2 | HIS | 1159 | 16.793 | 34.736 | 10.311 | 1.00 | 15.60 |
| ATOM | 2751 | CE1 | HIS | 1159 | 16.454 | 33.687 | 11.117 | 1.00 | 17.65 |
| ATOM | 2752 | N | LEU | 1160 | 19.356 | 35.526 | 14.930 | 1.00 | 12.27 |
| ATOM | 2753 | H | LEU | 1160 | 20.031 | 34.840 | 15.004 | 1.00 | 0.00 |
| ATOM | 2754 | CA | LEU | 1160 | 18.494 | 35.911 | 16.067 | 1.00 | 14.93 |
| ATOM | 2755 | CB | LEU | 1160 | 18.678 | 34.920 | 17.286 | 1.00 | 14.85 |
| ATOM | 2756 | CG | LEU | 1160 | 18.055 | 33.530 | 16.898 | 1.00 | 16.48 |
| ATOM | 2757 | CD1 | LEU | 1160 | 18.412 | 32.439 | 17.941 | 1.00 | 13.54 |
| ATOM | 2758 | CD2 | LEU | 1160 | 16.558 | 33.800 | 16.588 | 1.00 | 14.44 |
| ATOM | 2759 | C | LEU | 1160 | 18.744 | 37.325 | 16.524 | 1.00 | 14.89 |
| ATOM | 2760 | O | LEU | 1160 | 17.838 | 38.000 | 17.022 | 1.00 | 14.15 |
| ATOM | 2761 | N | GLY | 1161 | 19.965 | 37.825 | 16.385 | 1.00 | 15.89 |
| ATOM | 2762 | H | GLY | 1161 | 20.691 | 37.246 | 16.082 | 1.00 | 0.00 |
| ATOM | 2763 | CA | GLY | 1161 | 20.264 | 39.219 | 16.796 | 1.00 | 14.85 |
| ATOM | 2764 | C | GLY | 1161 | 19.556 | 40.169 | 15.835 | 1.00 | 14.93 |
| ATOM | 2765 | O | GLY | 1161 | 19.051 | 41.199 | 16.269 | 1.00 | 15.79 |
| ATOM | 2766 | N | ASN | 1162 | 19.517 | 39.818 | 14.536 | 1.00 | 13.58 |
| ATOM | 2767 | H | ASN | 1162 | 20.069 | 39.052 | 14.264 | 1.00 | 0.00 |
| ATOM | 2768 | CA | ASN | 1162 | 18.840 | 40.624 | 13.506 | 1.00 | 14.44 |
| ATOM | 2769 | CB | ASN | 1162 | 19.144 | 40.032 | 12.102 | 1.00 | 14.82 |
| ATOM | 2770 | CG | ASN | 1162 | 20.599 | 40.279 | 11.659 | 1.00 | 19.35 |
| ATOM | 2771 | OD1 | ASN | 1162 | 21.326 | 41.139 | 12.131 | 1.00 | 23.15 |
| ATOM | 2772 | ND2 | ASN | 1162 | 21.195 | 39.543 | 10.754 | 1.00 | 21.91 |
| ATOM | 2773 | HD21 | ASN | 1162 | 20.769 | 38.798 | 10.295 | 1.00 | 0.00 |
| ATOM | 2774 | HD22 | ASN | 1162 | 22.115 | 39.795 | 10.559 | 1.00 | 0.00 |
| ATOM | 2775 | C | ASN | 1162 | 17.322 | 40.619 | 13.751 | 1.00 | 14.81 |
| ATOM | 2776 | O | ASN | 1162 | 16.607 | 41.602 | 13.592 | 1.00 | 14.85 |
| ATOM | 2777 | N | LEU | 1163 | 16.788 | 39.429 | 14.035 | 1.00 | 13.70 |
| ATOM | 2778 | H | LEU | 1163 | 17.372 | 38.649 | 14.011 | 1.00 | 0.00 |
| ATOM | 2779 | CA | LEU | 1163 | 15.367 | 39.268 | 14.324 | 1.00 | 13.89 |
| ATOM | 2780 | CB | LEU | 1163 | 15.071 | 37.755 | 14.500 | 1.00 | 14.24 |
| ATOM | 2781 | CG | LEU | 1163 | 14.297 | 36.982 | 13.364 | 1.00 | 17.75 |
| ATOM | 2782 | CD1 | LEU | 1163 | 14.805 | 37.374 | 11.959 | 1.00 | 16.20 |
| ATOM | 2783 | CD2 | LEU | 1163 | 14.416 | 35.468 | 13.584 | 1.00 | 12.76 |
| ATOM | 2784 | C | LEU | 1163 | 15.076 | 40.082 | 15.582 | 1.00 | 17.98 |
| ATOM | 2785 | O | LEU | 1163 | 14.177 | 40.894 | 15.603 | 1.00 | 19.46 |
| ATOM | 2786 | N | LEU | 1164 | 15.836 | 40.030 | 16.669 | 1.00 | 19.64 |
| ATOM | 2787 | H | LEU | 1164 | 16.605 | 39.428 | 16.668 | 1.00 | 0.00 |
| ATOM | 2788 | CA | LEU | 1164 | 15.528 | 40.831 | 17.870 | 1.00 | 19.91 |
| ATOM | 2789 | CB | LEU | 1164 | 16.597 | 40.506 | 18.942 | 1.00 | 17.11 |
| ATOM | 2790 | CG | LEU | 1164 | 16.465 | 41.267 | 20.276 | 1.00 | 17.98 |
| ATOM | 2791 | CD1 | LEU | 1164 | 15.046 | 41.152 | 20.974 | 1.00 | 13.53 |
| ATOM | 2792 | CD2 | LEU | 1164 | 17.622 | 40.705 | 21.108 | 1.00 | 16.76 |
| ATOM | 2793 | C | LEU | 1164 | 15.474 | 42.332 | 17.555 | 1.00 | 21.45 |
| ATOM | 2794 | O | LEU | 1164 | 14.639 | 43.081 | 18.099 | 1.00 | 20.88 |
| ATOM | 2795 | N | GLN | 1165 | 16.497 | 42.748 | 16.797 | 1.00 | 21.33 |
| ATOM | 2796 | H | GLN | 1165 | 17.204 | 42.118 | 16.572 | 1.00 | 0.00 |
| ATOM | 2797 | CA | GLN | 1165 | 16.631 | 44.141 | 16.342 | 1.00 | 23.29 |
| ATOM | 2798 | CB | GLN | 1165 | 17.898 | 44.297 | 15.431 | 1.00 | 22.92 |
| ATOM | 2799 | CG | GLN | 1165 | 18.122 | 45.705 | 14.864 | 1.00 | 24.62 |
| ATOM | 2800 | CD | GLN | 1165 | 17.740 | 46.823 | 15.855 | 1.00 | 25.73 |
| ATOM | 2801 | OE1 | GLN | 1165 | 18.074 | 46.914 | 17.053 | 1.00 | 26.75 |
| ATOM | 2802 | NE2 | GLN | 1165 | 17.001 | 47.779 | 15.341 | 1.00 | 23.23 |
| ATOM | 2803 | HE21 | GLN | 1165 | 16.718 | 47.764 | 14.414 | 1.00 | 0.00 |
| ATOM | 2804 | HE22 | GLN | 1165 | 16.742 | 48.485 | 15.961 | 1.00 | 0.00 |
| ATOM | 2805 | C | GLN | 1165 | 15.368 | 44.522 | 15.563 | 1.00 | 23.93 |
| ATOM | 2806 | O | GLN | 1165 | 14.884 | 45.657 | 15.674 | 1.00 | 25.04 |
| ATOM | 2807 | N | ALA | 1166 | 14.905 | 43.635 | 14.689 | 1.00 | 23.83 |
| ATOM | 2808 | H | ALA | 1166 | 15.212 | 42.712 | 14.682 | 1.00 | 0.00 |
| ATOM | 2809 | CA | ALA | 1166 | 13.659 | 44.020 | 14.020 | 1.00 | 25.01 |
| ATOM | 2810 | CB | ALA | 1166 | 13.524 | 43.007 | 12.811 | 1.00 | 17.81 |
| ATOM | 2811 | C | ALA | 1166 | 12.351 | 44.186 | 14.861 | 1.00 | 26.97 |
| ATOM | 2812 | O | ALA | 1166 | 12.135 | 43.827 | 16.056 | 1.00 | 28.99 |
| ATOM | 2814 | C01 | AGI | 1 | 14.657 | 29.228 | 41.517 | 1.00 | 12.23 |
| ATOM | 2815 | C02 | AGI | 1 | 14.888 | 27.853 | 41.286 | 1.00 | 10.52 |
| ATOM | 2816 | N03 | AGI | 1 | 13.872 | 26.949 | 41.331 | 1.00 | 14.11 |
| ATOM | 2817 | C04 | AGI | 1 | 12.601 | 27.333 | 41.596 | 1.00 | 13.51 |
| ATOM | 2818 | C05 | AGI | 1 | 12.294 | 28.701 | 41.836 | 1.00 | 15.19 |
| ATOM | 2819 | C06 | AGI | 1 | 13.344 | 29.663 | 41.802 | 1.00 | 14.87 |
| ATOM | 2820 | C07 | AGI | 1 | 20.645 | 24.445 | 38.653 | 1.00 | 12.96 |
| ATOM | 2821 | C08 | AGI | 1 | 19.245 | 24.269 | 38.867 | 1.00 | 14.35 |
| ATOM | 2822 | C09 | AGI | 1 | 18.433 | 25.295 | 39.396 | 1.00 | 13.86 |
| ATOM | 2823 | C10 | AGI | 1 | 19.059 | 26.520 | 39.713 | 1.00 | 12.39 |
| ATOM | 2824 | C11 | AGI | 1 | 20.463 | 26.702 | 39.508 | 1.00 | 12.85 |
| ATOM | 2825 | C12 | AGI | 1 | 21.247 | 25.678 | 38.988 | 1.00 | 13.36 |
| ATOM | 2826 | C13 | AGI | 1 | 18.625 | 27.749 | 40.235 | 1.00 | 11.17 |
| ATOM | 2827 | N14 | AGI | 1 | 19.686 | 28.609 | 40.341 | 1.00 | 11.91 |
| ATOM | 2828 | N15 | AGI | 1 | 20.808 | 27.957 | 39.885 | 1.00 | 11.20 |
| ATOM | 2829 | C16 | AGI | 1 | 17.241 | 28.211 | 40.676 | 1.00 | 10.07 |
| ATOM | 2830 | C17 | AGI | 1 | 16.195 | 27.381 | 40.784 | 1.00 | 10.09 |
| ATOM | 2831 | C18 | AGI | 1 | 22.894 | 23.344 | 38.259 | 1.00 | 7.74 |
| ATOM | 2832 | C19 | AGI | 1 | 23.458 | 23.542 | 39.517 | 1.00 | 7.49 |
| ATOM | 2833 | C20 | AGI | 1 | 24.812 | 23.276 | 39.756 | 1.00 | 9.47 |
| ATOM | 2834 | C21 | AGI | 1 | 25.637 | 22.823 | 38.688 | 1.00 | 10.66 |
| ATOM | 2835 | C22 | AGI | 1 | 25.060 | 22.678 | 37.389 | 1.00 | 9.86 |
| ATOM | 2836 | C23 | AGI | 1 | 23.697 | 22.931 | 37.224 | 1.00 | 9.43 |
| ATOM | 2837 | C24 | AGI | 1 | 21.454 | 23.439 | 38.008 | 1.00 | 10.64 |
| ATOM | 2838 | O25 | AGI | 1 | 20.907 | 22.657 | 37.228 | 1.00 | 11.90 |
| ATOM | 2839 | N26 | AGI | 1 | 25.810 | 22.349 | 36.304 | 1.00 | 12.00 |
| ATOM | 2840 | C27 | AGI | 1 | 25.706 | 22.897 | 35.080 | 1.00 | 11.76 |
| ATOM | 2841 | O28 | AGI | 1 | 24.860 | 23.766 | 34.843 | 1.00 | 14.03 |
| ATOM | 2842 | C29 | AGI | 1 | 26.533 | 22.403 | 34.084 | 1.00 | 11.69 |
| ATOM | 2843 | N30 | AGI | 1 | 26.886 | 23.012 | 32.888 | 1.00 | 13.97 |
| ATOM | 2844 | N31 | AGI | 1 | 27.721 | 22.093 | 32.207 | 1.00 | 15.02 |
| ATOM | 2845 | C33 | AGI | 1 | 27.845 | 20.979 | 32.956 | 1.00 | 14.21 |
| ATOM | 2846 | C34 | AGI | 1 | 27.122 | 21.150 | 34.128 | 1.00 | 12.95 |
| ATOM | 2847 | C50 | AGI | 1 | 26.442 | 24.302 | 32.306 | 1.00 | 12.41 |
| ATOM | 2848 | C61 | AGI | 1 | 28.507 | 19.753 | 32.483 | 1.00 | 14.39 |
| ATOM | 2849 | C62 | AGI | 1 | 27.491 | 25.341 | 31.972 | 1.00 | 12.87 |
| ATOM | 2850 | C63 | AGI | 1 | 27.040 | 22.419 | 39.025 | 1.00 | 4.72 |
| ATOM | 2851 | C64 | AGI | 1 | 25.624 | 24.082 | 31.015 | 1.00 | 9.99 |
| ATOM | 2852 | H46 | AGI | 1 | 21.707 | 28.342 | 39.868 | 1.00 | 0.00 |
| ATOM | 2855 | H52 | AGI | 1 | 26.503 | 21.681 | 36.437 | 1.00 | 0.00 |
| ATOM | 2856 | OH2 | WAT | 1 | 21.326 | 15.278 | 31.552 | 1.00 | 40.62 |
| ATOM | 2859 | OH2 | WAT | 2 | 21.122 | 12.135 | 34.733 | 1.00 | 20.69 |
| ATOM | 2862 | OH2 | WAT | 3 | 19.111 | 11.893 | 36.318 | 1.00 | 28.54 |
| ATOM | 2865 | OH2 | WAT | 4 | 36.002 | 23.269 | 51.395 | 1.00 | 10.20 |
| ATOM | 2868 | OH2 | WAT | 5 | 27.957 | 15.413 | 47.018 | 1.00 | 6.01 |
| ATOM | 2871 | OH2 | WAT | 6 | 30.208 | 3.049 | 45.574 | 1.00 | 25.23 |
| ATOM | 2874 | OH2 | WAT | 7 | 35.908 | 6.812 | 36.342 | 1.00 | 24.75 |
| ATOM | 2877 | OH2 | WAT | 8 | 12.089 | 21.268 | 28.643 | 1.00 | 11.91 |
| ATOM | 2880 | OH2 | WAT | 9 | 8.284 | 23.360 | 27.605 | 1.00 | 13.03 |
| ATOM | 2883 | OH2 | WAT | 10 | 7.168 | 15.310 | 27.797 | 1.00 | 18.13 |
| ATOM | 2886 | OH2 | WAT | 11 | −2.983 | 28.550 | 27.886 | 1.00 | 15.16 |
| ATOM | 2889 | OH2 | WAT | 12 | −2.275 | 37.088 | 28.131 | 1.00 | 22.52 |
| ATOM | 2892 | OH2 | WAT | 13 | 14.309 | 38.611 | 34.575 | 1.00 | 19.70 |
| ATOM | 2895 | OH2 | WAT | 14 | 7.291 | 42.804 | 21.456 | 1.00 | 19.86 |
| ATOM | 2898 | OH2 | WAT | 15 | 9.260 | 21.856 | 32.707 | 1.00 | 38.34 |
| ATOM | 2901 | OH2 | WAT | 16 | 14.114 | 32.622 | 9.695 | 1.00 | 21.03 |
| ATOM | 2904 | OH2 | WAT | 17 | 10.414 | 37.234 | 13.886 | 1.00 | 22.43 |
| ATOM | 2907 | OH2 | WAT | 18 | 29.146 | 37.239 | 28.517 | 1.00 | 17.72 |
| ATOM | 2910 | OH2 | WAT | 19 | 35.368 | 33.301 | 30.265 | 1.00 | 16.57 |
| ATOM | 2913 | OH2 | WAT | 20 | 23.729 | 16.727 | 21.263 | 1.00 | 16.27 |
| ATOM | 2916 | OH2 | WAT | 21 | 18.412 | 16.139 | 23.628 | 1.00 | 8.38 |
| ATOM | 2919 | OH2 | WAT | 22 | 20.274 | 7.299 | 21.586 | 1.00 | 39.84 |
| ATOM | 2922 | OH2 | WAT | 23 | 15.089 | 22.125 | 7.341 | 1.00 | 29.02 |
| ATOM | 2925 | OH2 | WAT | 24 | 29.968 | 13.621 | 47.542 | 1.00 | 7.11 |
| ATOM | 2928 | OH2 | WAT | 25 | 36.009 | 29.181 | 57.592 | 1.00 | 27.24 |
| ATOM | 2931 | OH2 | WAT | 26 | 9.884 | 20.414 | 30.164 | 1.00 | 15.48 |
| ATOM | 2934 | OH2 | WAT | 27 | 1.007 | 22.249 | 27.229 | 1.00 | 17.18 |
| ATOM | 2937 | OH2 | WAT | 28 | 13.415 | 13.833 | 18.213 | 1.00 | 13.87 |
| ATOM | 2940 | OH2 | WAT | 29 | 19.990 | 15.760 | 19.898 | 1.00 | 23.23 |
| ATOM | 2943 | OH2 | WAT | 30 | 1.586 | 26.470 | 20.830 | 1.00 | 9.68 |
| ATOM | 2946 | OH2 | WAT | 31 | 32.958 | 30.321 | 36.397 | 1.00 | 14.63 |
| ATOM | 2949 | OH2 | WAT | 32 | 36.163 | 31.069 | 24.083 | 1.00 | 59.32 |
| ATOM | 2952 | OH2 | WAT | 33 | 14.712 | 18.180 | 39.370 | 1.00 | 30.96 |
| ATOM | 2955 | OH2 | WAT | 34 | 19.488 | 37.646 | 9.614 | 1.00 | 33.22 |
| ATOM | 2958 | OH2 | WAT | 35 | 11.039 | 12.958 | 25.121 | 1.00 | 17.87 |
| ATOM | 2961 | OH2 | WAT | 36 | 37.210 | 29.020 | 34.742 | 1.00 | 19.32 |
| ATOM | 2964 | OH2 | WAT | 37 | 13.045 | 15.428 | 38.169 | 1.00 | 37.63 |
| ATOM | 2967 | OH2 | WAT | 38 | 10.982 | 21.290 | 34.600 | 1.00 | 24.69 |
| ATOM | 2970 | OH2 | WAT | 39 | 19.683 | 12.262 | 51.020 | 1.00 | 18.71 |
| ATOM | 2973 | OH2 | WAT | 40 | 21.744 | 37.281 | 46.695 | 1.00 | 22.93 |
| ATOM | 2976 | OH2 | WAT | 41 | 28.788 | 33.762 | 22.409 | 1.00 | 13.24 |
| ATOM | 2979 | OH2 | WAT | 42 | 27.295 | 25.113 | 15.877 | 1.00 | 19.50 |
| ATOM | 2982 | OH2 | WAT | 43 | 4.156 | 8.840 | 24.882 | 1.00 | 44.83 |
| ATOM | 2985 | OH2 | WAT | 44 | 7.599 | 20.677 | 28.597 | 1.00 | 22.41 |
| ATOM | 2988 | OH2 | WAT | 45 | 4.099 | 26.412 | 35.222 | 1.00 | 26.02 |
| ATOM | 2991 | OH2 | WAT | 46 | −5.126 | 26.885 | 28.174 | 1.00 | 8.28 |
| ATOM | 2994 | OH2 | WAT | 47 | 14.163 | 41.346 | 37.612 | 1.00 | 36.83 |
| ATOM | 2997 | OH2 | WAT | 48 | 15.403 | 30.973 | 7.890 | 1.00 | 30.54 |
| ATOM | 3000 | OH2 | WAT | 49 | 19.092 | 31.462 | 8.139 | 1.00 | 58.96 |
| ATOM | 3003 | OH2 | WAT | 50 | 17.155 | 17.661 | 25.588 | 1.00 | 11.94 |
| ATOM | 3006 | OH2 | WAT | 51 | 21.191 | 18.666 | 23.398 | 1.00 | 10.12 |
| ATOM | 3009 | OH2 | WAT | 52 | 26.582 | 14.171 | 12.746 | 1.00 | 16.95 |
| ATOM | 3012 | OH2 | WAT | 53 | 1.621 | 33.696 | 15.903 | 1.00 | 40.51 |
| ATOM | 3015 | OH2 | WAT | 54 | 23.133 | 33.673 | 10.092 | 1.00 | 11.44 |
| ATOM | 3018 | OH2 | WAT | 55 | 0.665 | 22.521 | 22.322 | 1.00 | 17.92 |
| ATOM | 3021 | OH2 | WAT | 56 | 8.571 | 12.272 | 26.250 | 1.00 | 14.87 |
| ATOM | 3024 | OH2 | WAT | 57 | 8.739 | 41.024 | 32.785 | 1.00 | 41.13 |
| ATOM | 3027 | OH2 | WAT | 58 | 13.568 | 21.758 | 35.139 | 1.00 | 32.58 |
| ATOM | 3030 | OH2 | WAT | 59 | 6.710 | 22.465 | 36.178 | 1.00 | 27.76 |
| ATOM | 3033 | OH2 | WAT | 60 | 16.651 | 28.180 | 50.999 | 1.00 | 16.71 |
| ATOM | 3036 | OH2 | WAT | 61 | 20.228 | 29.243 | 53.230 | 1.00 | 22.89 |
| ATOM | 3039 | OH2 | WAT | 62 | 24.339 | 24.401 | 56.759 | 1.00 | 42.24 |
| ATOM | 3042 | OH2 | WAT | 63 | 29.898 | 36.602 | 47.827 | 1.00 | 15.75 |
| ATOM | 3045 | OH2 | WAT | 64 | 25.709 | 37.413 | 54.297 | 1.00 | 2.79 |
| ATOM | 3048 | OH2 | WAT | 65 | 23.280 | 41.339 | 53.434 | 1.00 | 46.05 |
| ATOM | 3051 | OH2 | WAT | 66 | 24.076 | 40.573 | 50.819 | 1.00 | 29.20 |
| ATOM | 3054 | OH2 | WAT | 67 | 22.373 | 34.699 | 38.292 | 1.00 | 21.93 |
| ATOM | 3057 | OH2 | WAT | 68 | 10.070 | 38.965 | 33.761 | 1.00 | 42.65 |
| ATOM | 3060 | OH2 | WAT | 69 | 1.837 | 20.637 | 23.877 | 1.00 | 17.56 |
| ATOM | 3063 | OH2 | WAT | 70 | −0.302 | 24.519 | 24.058 | 1.00 | 30.58 |
| ATOM | 3066 | OH2 | WAT | 71 | 16.620 | 24.213 | 6.070 | 1.00 | 53.95 |
| ATOM | 3069 | OH2 | WAT | 72 | 29.377 | 23.172 | 13.079 | 1.00 | 29.35 |
| ATOM | 3072 | OH2 | WAT | 73 | 32.068 | 32.850 | 22.056 | 1.00 | 33.38 |
| ATOM | 3075 | OH2 | WAT | 74 | 29.189 | 36.119 | 23.475 | 1.00 | 31.79 |
| ATOM | 3078 | OH2 | WAT | 75 | 30.528 | 14.083 | 34.616 | 1.00 | 33.71 |
| ATOM | 3081 | OH2 | WAT | 76 | 18.803 | 15.430 | 33.887 | 1.00 | 22.21 |
| ATOM | 3084 | OH2 | WAT | 77 | 30.636 | 3.117 | 42.678 | 1.00 | 24.91 |
| ATOM | 3087 | OH2 | WAT | 78 | 35.618 | 31.102 | 35.443 | 1.00 | 12.62 |
| ATOM | 3090 | OH2 | WAT | 79 | 35.412 | 30.339 | 46.030 | 1.00 | 22.58 |
| ATOM | 3093 | OH2 | WAT | 80 | 33.533 | 13.470 | 49.934 | 1.00 | 38.40 |
| ATOM | 3096 | OH2 | WAT | 81 | 35.138 | 15.589 | 47.560 | 1.00 | 49.63 |
| ATOM | 3099 | OH2 | WAT | 82 | 17.684 | 35.293 | 47.161 | 1.00 | 50.48 |
| ATOM | 3102 | OH2 | WAT | 83 | 21.563 | 35.750 | 44.071 | 1.00 | 41.03 |
| ATOM | 3105 | OH2 | WAT | 84 | 22.272 | 35.570 | 41.561 | 1.00 | 33.49 |
| ATOM | 3108 | OH2 | WAT | 85 | 20.337 | 14.435 | 23.547 | 1.00 | 18.84 |
| ATOM | 3111 | OH2 | WAT | 86 | 18.635 | 35.567 | 40.040 | 1.00 | 20.39 |
| ATOM | 3114 | OH2 | WAT | 87 | 12.484 | 16.399 | 17.879 | 1.00 | 13.75 |
| ATOM | 3117 | OH2 | WAT | 88 | 9.684 | 19.007 | 14.941 | 1.00 | 11.44 |
| ATOM | 3120 | OH2 | WAT | 89 | 25.317 | 15.393 | 14.846 | 1.00 | 19.23 |
| ATOM | 3123 | OH2 | WAT | 90 | 20.853 | 5.113 | 10.550 | 1.00 | 42.74 |
| ATOM | 3126 | OH2 | WAT | 91 | 19.040 | 24.627 | 7.686 | 1.00 | 40.90 |
| ATOM | 3129 | OH2 | WAT | 92 | 2.982 | 19.599 | 16.594 | 1.00 | 22.53 |
| ATOM | 3132 | OH2 | WAT | 93 | 14.681 | 24.573 | 40.178 | 1.00 | 16.19 |
| ATOM | 3135 | OH2 | WAT | 94 | 12.896 | 25.375 | 38.166 | 1.00 | 16.64 |
| ATOM | 3138 | OH2 | WAT | 95 | 10.421 | 26.185 | 38.637 | 1.00 | 19.63 |
| ATOM | 3141 | OH2 | WAT | 96 | 10.508 | 28.355 | 36.780 | 1.00 | 7.73 |
| ATOM | 3144 | OH2 | WAT | 97 | 24.817 | 16.201 | 37.345 | 1.00 | 13.66 |
| ATOM | 3147 | OH2 | WAT | 98 | 19.583 | 17.862 | 32.567 | 1.00 | 30.31 |
| ATOM | 3150 | OH2 | WAT | 99 | 36.696 | 32.185 | 32.118 | 1.00 | 43.55 |
| ATOM | 3153 | OH2 | WAT | 100 | 30.378 | 29.467 | 11.185 | 1.00 | 15.80 |
| ATOM | 3156 | OH2 | WAT | 101 | 17.522 | 20.998 | 51.523 | 1.00 | 20.67 |
| ATOM | 3159 | OH2 | WAT | 102 | −2.246 | 31.648 | 34.359 | 1.00 | 25.96 |
| ATOM | 3162 | OH2 | WAT | 103 | 41.235 | 27.544 | 37.500 | 1.00 | 35.27 |
| ATOM | 3165 | OH2 | WAT | 104 | 42.883 | 15.602 | 37.183 | 1.00 | 22.18 |
| ATOM | 3168 | OH2 | WAT | 105 | 20.640 | 30.814 | 10.293 | 1.00 | 28.02 |
| ATOM | 3171 | OH2 | WAT | 106 | 18.246 | 30.180 | 10.791 | 1.00 | 33.19 |
| ATOM | 3174 | OH2 | WAT | 107 | 25.751 | 19.552 | 29.534 | 1.00 | 13.87 |
| ATOM | 3177 | OH2 | WAT | 108 | 31.295 | 36.890 | 31.258 | 1.00 | 33.53 |
| ATOM | 3180 | OH2 | WAT | 109 | 27.712 | 21.370 | 29.184 | 1.00 | 18.70 |
| ATOM | 3183 | OH2 | WAT | 110 | 27.435 | 14.737 | 36.228 | 1.00 | 52.00 |
| ATOM | 3186 | OH2 | WAT | 111 | 21.338 | 39.310 | 35.683 | 1.00 | 49.00 |
| ATOM | 3189 | OH2 | WAT | 112 | 23.556 | 37.754 | 35.494 | 1.00 | 72.91 |
| ATOM | 3192 | OH2 | WAT | 113 | 26.958 | 35.986 | 34.773 | 1.00 | 29.61 |
| ATOM | 3195 | OH2 | WAT | 114 | 29.712 | 36.848 | 33.733 | 1.00 | 46.80 |
| ATOM | 3198 | OH2 | WAT | 115 | 32.223 | 28.024 | 35.019 | 1.00 | 10.03 |
| ATOM | 3201 | OH2 | WAT | 116 | 28.836 | 28.180 | 16.413 | 1.00 | 28.80 |
| ATOM | 3204 | OH2 | WAT | 117 | 26.242 | 24.136 | 9.219 | 1.00 | 26.46 |
| ATOM | 3207 | OH2 | WAT | 118 | 37.076 | 24.349 | 55.142 | 1.00 | 53.88 |
| ATOM | 3210 | OH2 | WAT | 119 | 16.584 | 18.239 | 50.274 | 1.00 | 50.32 |
| ATOM | 3213 | OH2 | WAT | 120 | 20.687 | 19.041 | 52.502 | 1.00 | 60.59 |
| ATOM | 3216 | OH2 | WAT | 121 | 19.388 | 11.344 | 30.526 | 1.00 | 31.54 |
| ATOM | 3219 | OH2 | WAT | 122 | 13.781 | 27.181 | 48.254 | 1.00 | 48.78 |
| ATOM | 3222 | OH2 | WAT | 123 | 30.389 | 27.761 | 33.210 | 1.00 | 21.69 |
| ATOM | 3225 | OH2 | WAT | 124 | 40.253 | 28.434 | 33.917 | 1.00 | 75.10 |
| ATOM | 3228 | OH2 | WAT | 125 | 39.462 | 21.892 | 34.709 | 1.00 | 19.15 |
| ATOM | 3231 | OH2 | WAT | 126 | 27.354 | 16.631 | 28.101 | 1.00 | 22.84 |
| ATOM | 3234 | OH2 | WAT | 127 | 37.264 | 13.998 | 45.657 | 1.00 | 40.56 |
| ATOM | 3237 | OH2 | WAT | 128 | 35.959 | 12.392 | 43.394 | 1.00 | 41.27 |
| ATOM | 3240 | OH2 | WAT | 129 | 36.794 | 12.046 | 40.541 | 1.00 | 72.34 |
| ATOM | 3243 | OH2 | WAT | 130 | 37.245 | 13.504 | 37.819 | 1.00 | 24.43 |
| ATOM | 3246 | OH2 | WAT | 131 | 36.721 | 10.256 | 37.335 | 1.00 | 42.59 |
| ATOM | 3249 | OH2 | WAT | 132 | 40.288 | 11.871 | 37.990 | 1.00 | 36.66 |
| ATOM | 3252 | OH2 | WAT | 133 | 24.153 | 7.378 | 49.364 | 1.00 | 25.67 |
| ATOM | 3255 | OH2 | WAT | 134 | 19.986 | 11.143 | 39.784 | 1.00 | 10.74 |
| ATOM | 3258 | OH2 | WAT | 135 | 19.218 | 9.612 | 37.696 | 1.00 | 31.06 |
| ATOM | 3261 | OH2 | WAT | 136 | 16.245 | 9.526 | 38.034 | 1.00 | 44.29 |
| ATOM | 3264 | OH2 | WAT | 137 | 11.126 | 18.199 | 32.681 | 1.00 | 30.63 |
| ATOM | 3267 | OH2 | WAT | 138 | 7.259 | 18.098 | 27.972 | 1.00 | 21.94 |
| ATOM | 3270 | OH2 | WAT | 139 | −0.413 | 33.704 | 33.451 | 1.00 | 24.97 |
| ATOM | 3273 | OH2 | WAT | 140 | 12.755 | 20.682 | 9.178 | 1.00 | 11.64 |
| ATOM | 3276 | OH2 | WAT | 141 | 26.028 | 20.695 | 4.318 | 1.00 | 27.37 |
| ATOM | 3279 | OH2 | WAT | 142 | 34.962 | 22.629 | 53.842 | 1.00 | 61.06 |
| ATOM | 3282 | OH2 | WAT | 143 | 27.025 | 23.617 | 54.543 | 1.00 | 53.62 |
| ATOM | 3285 | OH2 | WAT | 144 | 7.555 | 12.566 | 14.146 | 1.00 | 26.52 |
| ATOM | 3288 | OH2 | WAT | 145 | 5.587 | 17.643 | 9.027 | 1.00 | 29.81 |
| ATOM | 3291 | OH2 | WAT | 146 | 29.852 | 36.461 | 40.397 | 1.00 | 35.57 |
| ATOM | 3294 | OH2 | WAT | 147 | 11.764 | 40.251 | 37.125 | 1.00 | 49.71 |
| ATOM | 3297 | OH2 | WAT | 148 | 5.458 | 33.528 | 37.234 | 1.00 | 42.83 |
| ATOM | 3300 | OH2 | WAT | 149 | 7.202 | 37.344 | 36.336 | 1.00 | 31.47 |
| ATOM | 3303 | OH2 | WAT | 150 | 4.077 | 13.900 | 22.925 | 1.00 | 34.83 |
| ATOM | 3306 | OH2 | WAT | 151 | 36.124 | 30.826 | 29.114 | 1.00 | 20.92 |
| ATOM | 3309 | OH2 | WAT | 152 | 32.404 | 35.448 | 28.112 | 1.00 | 37.67 |
| ATOM | 3312 | OH2 | WAT | 153 | 28.928 | 36.235 | 19.814 | 1.00 | 25.08 |
| ATOM | 3315 | OH2 | WAT | 154 | 31.185 | 28.578 | 18.639 | 1.00 | 31.69 |
| ATOM | 3318 | OH2 | WAT | 155 | 31.464 | 15.828 | 18.034 | 1.00 | 32.81 |
| ATOM | 3321 | OH2 | WAT | 156 | 28.801 | 10.653 | 19.297 | 1.00 | 11.68 |
| ATOM | 3324 | OH2 | WAT | 157 | 27.123 | 7.555 | 21.919 | 1.00 | 27.17 |
| ATOM | 3327 | OH2 | WAT | 158 | 26.267 | 4.503 | 15.012 | 1.00 | 16.48 |
| ATOM | 3330 | OH2 | WAT | 159 | 14.887 | 13.382 | 9.322 | 1.00 | 47.34 |
| ATOM | 3333 | OH2 | WAT | 160 | 15.567 | 28.507 | 5.142 | 1.00 | 35.81 |
| ATOM | 3336 | OH2 | WAT | 161 | 7.392 | 30.513 | 8.486 | 1.00 | 18.19 |
| ATOM | 3339 | OH2 | WAT | 162 | 1.107 | 30.087 | 15.076 | 1.00 | 25.99 |
| ATOM | 3342 | OH2 | WAT | 163 | −4.134 | 26.350 | 20.855 | 1.00 | 29.50 |
| ATOM | 3345 | OH2 | WAT | 164 | 4.528 | 15.330 | 28.802 | 1.00 | 41.12 |
| ATOM | 3348 | OH2 | WAT | 165 | 7.565 | 17.971 | 32.120 | 1.00 | 44.61 |
| ATOM | 3351 | OH2 | WAT | 166 | 31.483 | 16.529 | 33.377 | 1.00 | 27.78 |
| ATOM | 3354 | OH2 | WAT | 167 | 31.005 | 13.417 | 29.720 | 1.00 | 42.53 |
| ATOM | 3357 | OH2 | WAT | 168 | 34.705 | 12.405 | 31.083 | 1.00 | 30.74 |
| ATOM | 3360 | OH2 | WAT | 169 | 31.438 | 7.615 | 37.109 | 1.00 | 44.32 |
| ATOM | 3363 | OH2 | WAT | 170 | 9.475 | 31.639 | 38.599 | 1.00 | 30.55 |
| ATOM | 3366 | OH2 | WAT | 171 | 9.215 | 30.905 | 41.122 | 1.00 | 43.94 |
| ATOM | 3369 | OH2 | WAT | 172 | 7.419 | 29.700 | 37.818 | 1.00 | 27.59 |

TABLE 6
VEGFR2 Ligand Binding Site
Residues Within 5 Å of Ligand Atoms

| Amino Acid Residue | Compound 3 | Compound 4 | Compound 5 |
|---|---|---|---|
| L840 | + | + | + |
| V848 | + | + | + |
| E850 | + | | |
| A866 | + | + | + |
| V867 | | + | + |
| K868 | + | + | + |
| E885 | + | + | + |
| I888 | + | + | + |
| L889 | + | + | + |
| I892 | + | + | + |
| V898 | | + | + |
| V899 | + | + | + |
| V914 | + | + | + |
| V916 | + | + | + |
| E917 | + | + | + |
| F918 | + | + | + |
| C919 | + | + | + |
| K920 | + | + | + |
| F921 | | + | + |
| G922 | + | + | + |
| N923 | + | + | + |
| L1019 | + | + | + |
| C1024 | | + | |
| I1025 | | + | |
| H1026 | + | | + |
| L1035 | + | + | + |
| I1044 | + | + | + |
| C1045 | + | + | + |
| D1046 | + | + | + |
| F1047 | + | + | + |

### Example 5

### Use of VEGFR2KD:Compound 1 Co-Crystal Structure in the Design of New VEGFR Inhibitors

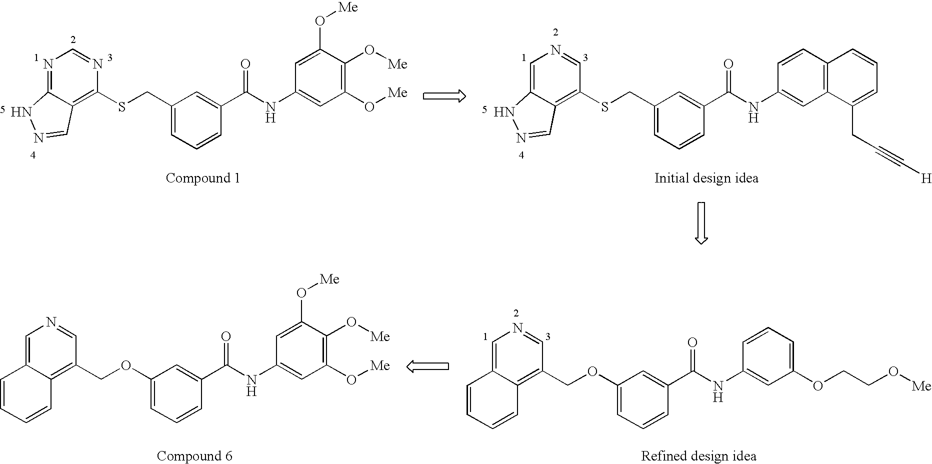

Using the program QUIKVU (developed by Agouron/Pfizer Pharamaceuticals, San Diego, Calif.), the co-crystal structure of Compound 1 bound to VEGFR2KD (See Table 1 for atomic coordinates) was displayed graphically and the key interactions of the ligand and the protein were analyzed. (QUIKVU was developed by Agouron/Pfizer Pharamaceuticals, San Diego, Calif. and is not commercially available. Other display programs that could be used include Quanta (Accelrys Inc., San Diego, Calif.), INSIGHT II (Accelrys Inc., San Diego, Calif.), or XTALVIEW (Scripps Reasearch Institute, La Jolla, Calif.)). A key observation was that N1 and N3 of Compound 6 were not making effective hydrogen bonds to the protein, as judged by both distance and geometrical considerations. On the other hand, C2 was within van der Waals radius (3.12 Å) of the backbone NH of Cys-919. Therefore, an initial design idea was to replace C2 with a nitrogen to function as a H-bond acceptor, and to replace N1 and N3 with carbon, to eliminate unfavorable desolvation penalties associated with binding to these H-bond acceptors. Computationally, this initial design idea was evaluated in the context of additional modifications to the “right-hand” anilide moiety, in that the trimethoxyphenyl group was replaced with a propargyl-naphthyl moiety. A further refinement was to replace the fused pyrazolyl moiety with a fused benzo ring, since N4 and N5 were not making H-bonds directly to the protein, with additional changes in the “right-hand” aryl group as well as the “linker” (—SCH₂— to —CH₂O—) in order to improve synthetic accessibility. These two designs (as well as the model for Compound 1) were sequentially submitted to the following computational model for estimating relative energetics of ligand binding:
(1) Using the Macromodel program suite (Schrodinger, Portland, Oreg.), a “substructure” of the protein was created from the crystallographic coordinates set forth in Table 1 which consisted of protein atoms (no solvent) within ca. 10 A of the inhibit.
(2) Energetic constraints were placed on the protein atoms not directly in contact with the inhibitor, in order that the active site would remain properly oriented during force field minimization, but still retain some flexibility to accommodate movement of the ligand.
(3) the ligand file, properly oriented in the active site in analogy to the crystallographically determined coordinates set forth in Table 1 of Compound 1, were merged with the protein file, and the resulting complex was submitted to Macromodel force field minimization using the AMBER* force field and the GBSA continuum solvation model using the BATCHMIN program. The resulting minimized energy was recorded and the coordinates of the minimized complex stored to file.
(4) the ligand was “extracted” from the minimized complex, and subjected to force field minimization by itself (AMBER*/GBSA/BATCHMIN). Once again, coordinates were saved and the energy recorded.
(5) The relative binding affinities were then predicted based on the formula:
   ΔE={E(ligand-complex)−E(ligand)}−{E(Compound 1-complex)−E(Compound 1)}
   For the initial design idea, ΔE was calculated to be −6.0 kcal/mol, relative to Compound 1, while the refined design idea gave a calculated value of −3.1 kcal/mol. In general, the calculated ΔE values generally overestimate actual differences by several-fold; nonetheless, the favorable energetics predicted from this modeling led to the prioritization of the refined design idea for subsequent synthesis. On the basis of synthetic considerations and the desire to examine single structural modifications sequentially, the first synthetic target completed, Compound 6, retained the “right-hand” trimethoxyphenyl group of Compound 1. Testing of Compound 6 in the enzyme ELISA assay, using phosphorylated VEGFR2KD, revealed it to be a potent inhibitor of VEGFR kinase activity with a Kᵢ of 138 nM.

### Example 6

### Activity Measurements: Enzyme Assays

Assays were used to determines the ability of a test compound to inhibit tyrosine kinase activity of VEGFR2KD. Autophosphorylation of the purified VEGFR2KD (SEQ ID NO: 3) was performed by incubation of the enzyme at a concentration of 4 μM in the presence of 3 mM ATP and 40 mM MgCl₂ in 100 mM HEPES, pH 7.5, containing 5% glycerol and 5 mM DTT, at 4° C. for 2 h. After autophosphorylation, the VEGFR2KD construct has been shown to possess catalytic activity essentially equivalent to the wild-type autophosphorylated kinase domain construct. See Parast et al., Biochemistry, 37, 16788-16801 (1998).

Coupled Spectrophotometric Assay

The production of ADP from ATP that accompanies phosphoryl transfer was coupled to oxidation of NADH using phosphoenolpyruvate (PEP) and a system having pyruvate kinase (PK) and lactic dehydrogenase (LDH). The oxidation of NADH was monitored by following the decrease of absorbance at 340 nm (e₃₄₀=6.22 cm⁻¹ mM⁻¹) using a Beckman DU 650 spectrophotometer. Assay conditions for phosphorylated VEGF-R2 KD were the following: 1 mM PEP; 250 μM NADH; 50 units of LDH/mL; 20 units of PK/mL; 5 mM DTT; 5.1 mM poly(E₄Y₁); 1 mM ATP; and 25 mM MgCl₂ in 200 mM HEPES, pH 7.5. Assay conditions for unphosphorylated VEGF-R2 KD (indicated as FLVK in the tables) were the following: 1 mM PEP; 250 μM NADH; 50 units of LDH/mL; 20 units of PK/mL; 5 mM DTT; 20 mM poly(E₄Y₁); 3 mM ATP; and 60 mM MgCl₂ and 2 mM MnCl₂ in 200 mM HEPES, pH 7.5. Assays were initiated with 5 to 40 nM of enzyme. Kᵢ values were determined by measuring enzyme activity in the presence of varying concentrations of test compounds. The data were analyzed using Enzyme Kinetic and Kaleidagraph software.

ELISA Assay

Formation of phosphogastrin was monitored using biotinylated gastrin peptide (1-17) as substrate. Biotinylated phosphogastrin was immobilized using streptavidin coated 96-well microtiter plates followed by detection using anti-phosphotyrosine-antibody conjugated to horseradish peroxidase. The activity of horseradish peroxidase was monitored using 2,2′-azino-di-[3-ethylbenzathiazoline sulfonate(6)]diammonium salt (ABTS). Typical assay solutions contained: 2 μM biotinylated gastrin peptide; 5 mM DTT; 20 μM ATP; 26 mM MgCl₂; and 2 mM MnCl₂ in 200 mM HEPES, pH 7.5. The assay was initiated with 0.8 nM of phosphorylated VEGF-R2 KD. Horseradish peroxidase activity was assayed using ABTS, 10 mM. The horseradish peroxidase reaction was quenched by addition of acid (H₂SO₄), followed by absorbance reading at 405 nm. Kᵢ values were determined by measuring enzyme activity in the presence of varying concentrations of test compounds. The data were analyzed using Enzyme Kinetic and Kaleidagraph software.

### Example 7

### Calculation of Sequence Alignments for VEGFR2 and VEGFR1 and VEGFR3

The protein sequences of human full-length VEGFR1 (SEQ ID NO: 4; Swiss-Prot Accession Number P17948), VEGFR2 (SEQ ID NO: 2; Swiss-Prot Accession Number P35968), and VEGFR3 (SEQ ID NO: 5; Swiss-Prot Accession Number P35916) were obtained from the Swiss-Prot Protein Knowledgebase (European Bioinformatics Institute, Cambridge, UK) using the internet site www.ebi.ac.uk/swissprot. The sequence regions encompassing the kinase domains, with the kinase insert domains included, were aligned using the Multiple Sequence Alignment tool in InsightII (98.0) (Accelrys, San Diego, Calif.). (See Table 7)

For the VEGFR2 amino acid residues 806-1171 of SEQ ID NO: 1, encompassing the kinase domain with the kinase insert domain included, the % identity to VEGFR1 is 73.84% and the percent identity to VEGFR3 is 67.75%. For the VEGFR2 catalytic kinase domain (amino acid residues 806-932 and 1001-1171 of SEQ ID NO: 1), the percent identity to VEGFR1 and VEGFR3 is 80.26% and 78.26% respectively. For residues that define the VEGFR2 ligand binding site (as defined above), the sequence identity to VEGFR1 and VEGFR3 is 90.00% and 96.66% respectively.

TABLE 7
Sequence Alignment of Human VEGFR Kinase Domains

VEGFR1: mdpdevpldeqcerlpydaskwefarerlklgkslgrgafgkvvqasafg
VEGFR2: mdpdelpldehcerlpydaskwefprdrlklgkplgrgafgqvieadafg (855)
VEGFR3: mdpgevpleeqceylsydasqwefprerlhlgrvlgygafgkvveasafg
VEGFR1: ikksptcrtvavkmlkegataseykalmtelkilthighhlnvvnllgac
VEGFR2: idktatcrtvavkmlkegathsehralmselkilihighhlnvvnllgac (905)
VEGFR3: ihkgsscdtvavkmlkegatasehralmselkilihignhlnvvnllgac
VEGFR1: tkqggplmviveyckygnlsnylkskrdlfflnkdaalhmepkkekmepg
VEGFR2: tkpggplmvivefckfgnlstylrskrnef-vpyktkgarfrqgkdyvga (954)
VEGFR3: tkpqgplmvivefckygnlsnflrakrdaf---spcaekspeqrgrfram
VEGFR1: leqgkkprldsvtssesfassgfqedkslsdveeeedsdgfykepitmed
VEGFR2: ipvdlkrrldsitssqssassgfveekslsdveeeeapedlykdfltleh (1004)
VEGFR3: velarldrrrpgssdrvlfarfskteggarraspdqeaedlwlspltmed
VEGFR1: lisysfqvargmeflssrkcihrdlaarnillsennvvkicdfglardiy
VEGFR2: licysfqvakgmeflasrkcihrdlaarnillseknvvkicdfglardiy (1054)
VEGFR3: lvcysfqvargmeflasrkcihrdlaarnillsesdvvkicdfglardiy
VEGFR1: knpdyvrkgdtrlplkwmapesifdkiystksdvwsygvllweifslggs
VEGFR2: kdpdyvrkgdarlplkwmapetifdrvytiqsdvwsfgvllweifslgas (1104)
VEGFR3: kdpdyvrkgsarlplkwmapesifdkvyttqsdvwsfgvllweifslgas
VEGFR1: pypgvqmdedfcsrlregmrmrapeystpeiyqimldcwhrdpkerprfa
VEGFR2: pypgvkideefcrrlkegtrmrapdyttpemyqtmldcwhgepsqrptfs (1154)
VEGFR3: pypgvqineefcqrlrdgtrmrapelatpairrimlncwsgdpkarpafs
VEGFR1: elveklgdllqanvqqd
VEGFR2: elvehlgnllqanaqqd (1171)
VEGFR3: elveilgdllqgrglqe

The practice of the present invention generally employs conventional techniques of molecular biology, microbiology, recombinant DNA, immunology, protein chemistry and crystallography, which are well-within the purview of the skilled artisan. Such techniques are explained fully in the literature. See, e.g., Sambrook et al., “Molecular Cloning: A Laboratory Manual,” 3^{rd} ed. (2001), Cold Spring Harbor Press, Cold Spring Harbor, N.Y.; Glover, ed., “DNA Cloning: A Practical Approach,” Volumes I and II, 2^{nd} (1995), IRL Press, Oxford, England; Ausbel et al., eds., “Current Protocols in Molecular Biology” (1994), Green Publishers Inc. and Wiley and Sons, New York, N.Y.; Innis et al., eds., “PCR Protocols: A Guide to Methods and Applications” (1990), Academic Press, San Diego, Calif.; Freshney “Culture of Animal Cells: A Manual of Basic Technique,” 4^{th} ed. (2000), Wiley & Sons, New York, N.Y.; and Perbal, “A Practical Guide to Molecular Cloning,” 2^{nd} ed. (1988), Wiley & Sons, New York, N.Y.

All articles, books, patents, patent applications and patent publications cited herein are incorporated by reference in their entirety.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A vascular endothelial growth factor receptor (VEGFR) comprising a ligand binding pocket that is defined by the atoms found in the structural coordinates set forth in Tables 1, 2, 3, 4 or 5, or in a related set of structural coordinates having a root mean square deviation of not more than about 0.90 Å away from the binding pocket Cα atoms of the ligand binding pocket defined by the atoms found in the structural coordinates set forth in Tables 1, 2, 3, 4 or 5.

2. The VEGFR according to claim 1, wherein the VEGFR is VEGFR2 kinase domain of SEQ ID NO: 3, or a conservatively substituted variant thereof.

3. An isolated peptide consisting of a ligand binding pocket that is defined by the atoms found in the structural coordinates set forth in Tables 1, 2, 3, 4 or 5, or in a related set of structural coordinates having a root mean square deviation of not more than about 0.90 Å away from the binding pocket Cα atoms of the ligand binding pocket defined by the atoms found in the structural coordinates set forth in Tables 1, 2, 3, 4 or 5.

4. A vascular endothelial growth factor receptor (VEGFR) comprising a ligand binding pocket that is of approximate dimensions 12 Å×9 Å×25 Å.

5. A vascular endothelial growth factor receptor (VEGFR) comprising a ligand binding pocket as depicted in FIG. 1, FIG. 2A, or FIG. 2B.

6. An isolated peptide consisting of a ligand binding pocket as depicted in FIG. 1, FIG. 2A, or FIG. 2B.

7. A vascular endothelial growth factor receptor (VEGFR) comprising a ligand binding pocket that is defined by the structural coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2 or a conservatively substituted variant thereof.

8. A vascular endothelial growth factor receptor (VEGFR) comprising an activation loop defined by amino acid residues 1046 to 1075 of SEQ ID NO: 2, or a conservatively substituted variant thereof, as depicted in FIG. 1.

9. An isolated peptide consisting of a ligand binding pocket that is defined by the structural coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2 or a conservatively substituted variant thereof.

10. An isolated peptide consisting of an activation loop defined by amino acid residues 1046 to 1075 of SEQ ID NO: 2, or a conservatively substituted variant thereof, as depicted in FIG.

11. A crystalline structure of a vascular endothelial growth factor receptor (VEGFR) peptide:ligand complex, wherein the crystalline structure is defined by the structural coordinates set forth in Tables 1, 2, 3, 4 or 5, or a related set of structural coordinates having a root mean square deviation of not more than about 1.25 Å away from the core Cα atoms of the structural coordinates set forth in Tables 1, 2, 3, 4 or 5.

12. The crystalline structure according to claim 11, wherein the VEGFR is VEGFR2 kinase domain of SEQ ID NO: 3, or a conservatively substituted variant thereof.

13. The crystalline structure according to claim 11, wherein the ligand is selected from the group consisting of: 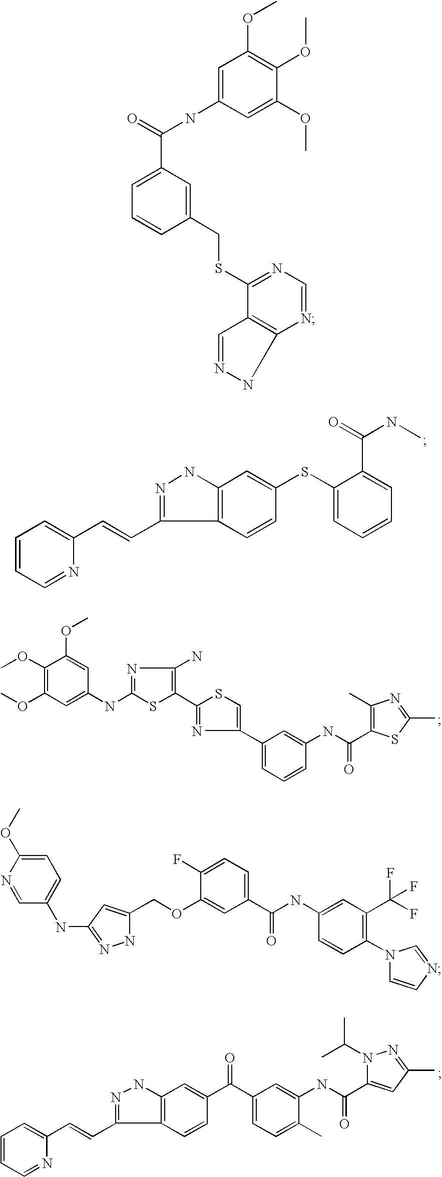
and mixtures thereof.

14. A crystalline structure of an isolated peptide:ligand complex, wherein the crystalline structure is defined by the structural coordinates set forth in Tables 1, 2, 3, 4 or 5, or a related set of structural coordinates having a root mean square deviation of not more than about 1.25 Å away from the core Cα atoms of the structural coordinates set forth in Tables 1, 2, 3, 4 or 5.

15. A crystalline structure of a vascular endothelial growth factor receptor (VEGFR) comprising a ligand binding pocket that is defined by the atoms found in the structural coordinates set forth in Tables 1, 2, 3, 4 or 5, or in a related set of structural coordinates having a root mean square deviation of not more than about 0.90 Å away from the binding pocket Cα atoms of the ligand binding pocket defined by the atoms found in the structural coordinates set forth in Tables 1, 2, 3, 4 or 5.

16. The crystalline structure according to claim 15, wherein the VEGFR is VEGFR2 kinase domain of SEQ ID NO: 3, or a conservatively substituted variant thereof.

17. A crystalline structure of an isolated peptide:ligand complex, wherein the peptide consists of a ligand binding pocket that is defined by the atoms found in the structural coordinates set forth in Tables 1, 2, 3, 4 or 5, or in a related set of structural coordinates having a root mean square deviation of not more than about 0.90 Å away from the binding pocket Cα atoms of the ligand binding pocket defined by the atoms found in the structural coordinates set forth in Tables 1, 2, 3, 4 or 5.

18. A crystalline structure of a vascular endothelial growth factor receptor (VEGFR):ligand complex, wherein the VEGFR comprises a ligand binding pocket that is of approximate dimensions 12 Å×9 Å×25 Å.

19. A crystalline structure of a vascular endothelial growth factor receptor (VEGFR):ligand complex, wherein the VEGFR comprises a ligand binding pocket as depicted in FIG. 1, FIG. 2A, or FIG. 2B.

20. A crystalline structure of an isolated peptide:ligand complex, wherein the peptide consists of a ligand binding pocket as depicted in FIG. 1, FIG. 2A, or FIG. 2B.

21. A crystalline structure of a vascular endothelial growth factor receptor (VEGFR):ligand complex, wherein the VEGFR comprises a ligand binding pocket that is defined by the structural coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2 or a conservatively substituted variant thereof.

22. A crystalline structure of a vascular endothelial growth factor receptor (VEGFR):ligand complex, wherein the VEGFR comprises an activation loop defined by amino acid residues 1046 to 1075 of SEQ ID NO: 2, or a conservatively substituted variant thereof, as depicted in FIG. 1.

23. A crystalline structure of an isolated peptide:ligand complex, wherein the peptide consists of a ligand binding pocket that is defined by the structural coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2 or a conservatively substituted variant thereof.

24. A crystalline structure of an isolated peptide:ligand complex, wherein the peptide consists of an activation loop defined by amino acid residues 1046 to 1075 of SEQ ID NO: 2, or a conservatively substituted variant thereof, as depicted in FIG. 1.

25. Three-dimensional structural coordinates of a peptide:ligand complex, comprising:
a vascular endothelial growth factor receptor (VEGFR), or a peptide that is structurally related thereto; and
a ligand,
wherein the complex has the atomic coordinates set forth in Tables 1, 2, 3, 4 or 5, or a related set of structural coordinates having a root mean square deviation of not more than about 1.25 Å away from the core Cα atoms of the structural coordinates as set forth in Tables 1, 2, 3, 4 or 5.

26. A method of utilizing molecular replacement to obtain structural information about a crystalline molecule or a crystalline molecular complex of unknown structure comprising:
generating an X-ray diffraction pattern from said crystallized molecule or molecular complex; and
applying at least a portion of the structural coordinates set forth in Tables 1, 2, 3, 4 or 5, or a related set of structural coordinates having a root mean square deviation of not more than about 1.25 Å away from the core Cα atoms of the structural coordinates as set forth in Tables 1, 2, 3, 4 or 5, to the X-ray diffraction pattern to generate a three-dimensional electron density map of at least a portion of the molecule or molecular complex whose structure is unknown.

27. A machine-readable medium having stored thereon data comprising the atomic coordinates as set forth in Tables 1, 2, 3, 4 or 5, or a related set of structural coordinates having a root mean square deviation of not more than about 1.25 Å away from the core Cα atoms of the structural coordinates as set forth in Tables 1, 2, 3, 4 or 5.

28. A method for generating a three-dimensional computer representation of a molecule comprising a vascular endothelial growth factor receptor kinase domain (VEGFRKD), or a peptide that is structurally related thereto, comprising applying the atomic coordinates set forth in Tables 1, 2, 3, 4 or 5, or a related set of atomic coordinates having a root mean square deviation of not more than about 1.25 Å away from the core Cα atoms of the atomic coordinates as set forth in Tables 1, 2, 3, 4 or 5, to a computer algorithm to generate a three-dimensional representation of the molecule.

29. A method for generating a three-dimensional computer representation of a vascular endothelial growth factor receptor (VEGFRKD) ligand binding pocket or VEGFRKD-like ligand binding pocket comprising applying the atomic coordinates set forth in Tables 1, 2, 3, 4 or 5, or a related set of atomic coordinates having a root mean square deviation of not more than about 0.90 Å away from the core Cα atoms of the atomic coordinates as set forth in Tables 1, 2, 3, 4 or 5, to a computer algorithm to generate a three-dimensional representation of the binding pocket.

30. The method according to claim 29, wherein the ligand binding pocket is defined by the structural coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2, or a conservatively substituted variant thereof.

31. A method of using a three-dimensional vascular endothelial growth factor receptor (VEGFR) kinase domain:ligand crystalline structure to identify a potential VEGFR modulator, comprising:
(a) selecting a potential modulator by performing rational drug design using a three-dimensional structure defined by at least a portion of the atomic coordinates set forth in Tables 1, 2, 3, 4 or 5, or a related set of atomic coordinates having a root mean square deviation of not more than about 1.25 Å away from the core Cα atoms of the atomic coordinates set forth in Tables 1, 2, 3, 4 or 5;
(b) contacting the potential modulator with a VEGFR polypeptide; and
(c) detecting whether the potential modulator binds with the polypeptide.

32. The method according to claim 31, wherein, wherein the selection is performed in conjunction with computer modeling.

32. The method according to claim 31, wherein the three-dimensional structure comprises a ligand binding pocket defined by the atomic coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2, or a conservatively substituted variant thereof.

33. A method for evaluating the potential of a chemical entity to associate with a vascular endothelial growth factor receptor kinase domain (VEGFRKD) ligand binding pocket or VEGFRKD-like ligand binding pocket comprising:
(a) employing computational means to perform a fitting operation between the chemical entity and a ligand binding pocket defined by at least a portion of the atomic coordinates set forth in Tables 1, 2, 3, 4 or 5, or a related set of atomic coordinates having a root mean square deviation of not more than about 0.90 Å away from the core Cα atoms of the atomic coordinates as set forth in Tables 1, 2, 3, 4 or 5; and
(b) analyzing the results of the fitting operation to quantify the association between the chemical entity and the binding pocket.

34. The method according to claim 33, wherein the ligand binding pocket is defined by the atomic coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2, or a conservatively substituted variant thereof.

35. A method for evaluating the ability of a chemical entity to associate with a molecule or molecular complex comprising a vascular endothelial growth factor receptor kinase domain (VEGFRKD) ligand binding pocket or VEGFRKD-like ligand binding pocket, comprising
(a) constructing a computer model of a ligand binding pocket defined by at least a portion of the atomic coordinates set forth in Tables 1, 2, 3, 4 or 5, or a related set of atomic coordinates having a root mean square deviation of not more than about 0.90 Å away from the core Cα atoms of the atomic coordinates as set forth in Tables 1, 2, 3, 4 or 5;
(b) selecting a compound to be evaluated by a method selected from the group consisting of: (i) assembling molecular fragments into a compound, (ii) selecting a compound from a small molecule database, (iii) de novo ligand design of a compound, and (iv) modifying a known modulator, or a portion thereof, of a VEGFR;
(c) employing computational means to perform a fitting program operation between a computer model of the compound to be evaluated and the computer model of the ligand binding pocket to provide an energy-minimized configuration of the compound in the binding pocket; and
(d) evaluating the results of the fitting operation to quantify the association between the compound and the binding pocket model.

36. The method according to claim 35, wherein the ligand binding pocket is defined by the atomic coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2, or a conservatively substituted variant thereof.

37. A method for identifying a modulator of a molecule comprising a vascular endothelial growth factor receptor kinase domain (VEGFRKD) ligand binding pocket or VEGRKD-like ligand binding pocket, comprising:
(a) generating a three-dimensional structure of the VEGFRKD or VEGFRKD-like ligand binding pocket by applying at least a portion of the atomic coordinates set forth in Tables 1, 2, 3, 4 or 5, or a related set of atomic coordinates having a root mean square deviation of not more than about 0.90 Å away from the core Cα atoms of the atomic coordinates as set forth in Tables 1, 2, 3, 4 or 5, to a computer algorithm to generate a three-dimensional structural representation of the binding pocket;
(b) employing the three-dimensional structure to design or select the modulator;
(c) synthesizing or obtaining the modulator; and
(d) contacting the modulator with the molecule to determine the ability of the modulator to interact with the molecule.

38. The method according to claim 37, wherein the ligand binding pocket is defined by the atomic coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2, or a conservatively substituted variant thereof.

39. A method for identifying a modulator of a molecule comprising a vascular endothelial growth factor receptor kinase domain (VEGFRKD) ligand binding pocket or VEGFRKD-like ligand binding pocket, comprising:
(a) constructing a computer model of the binding pocket;
(b) selecting a compound to be evaluated as a modulator by a method selected from the group consisting of: (i) assembling molecular fragments into a compound, (ii) selecting a compound from a small molecule database, (iii) de novo ligand design of a compound, and (iv) modifying a known inhibitor, or a portion thereof, of a VEGFR polypeptide;
(c) employing computational means to perform a fitting program operation between computer models of the compound to be evaluated and the binding pocket in order to provide an energy-minimized configuration of the compound in the binding pocket;
(d) evaluating the results of the fitting operation to quantity the association between the compound and the binding pocket model;
(e) synthesizing the compound; and
(f) contacting the compound with the molecule to determine the ability of the compound to modulate the kinase activity of the molecule,
wherein the ligand binding pocket is defined by at least a portion of the atomic coordinates set forth in Tables 1, 2, 3, 4 or 5, or a related set of atomic coordinates having a root mean square deviation of not more than about 0.90 Å away from the core Cα atoms of the atomic coordinates set forth in Tables 1, 2, 3, 4 or 5.

40. The method according to claim 41, wherein the ligand binding pocket is defined by the atomic coordinates of the following amino acid residues: L840, V848, E850, A866, V867, K868, E885, I888, L889, I892, V898, V899, V914, V916, E917, F918, K920, F921, N923, L1019, C1024, I1025, H1026, L1035, I1044, C1045, D1046, and F1047 of SEQ ID NO: 2, or a conservatively substituted variant thereof.


SEQUENCE LISTING (11 sequences)

SEQ ID NO: 1 - DNA, 5830 bp, Homo sapiens
actgagtcccgggaccccgggagagcggtcagtgtgtggtcgctgcgtttcctctgcctgcgccgggcatcacttgcgcgccgcagaaagtccgtctggcagcctggatatcctctcctaccggcacccgcagacgcccctgcagccgccggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctcttttttcattcatttaaaaagcattatcatgcccctgctgcgggtctcaccatgggtttagaacaaagagcttcaagcaatggccccatcctcaaagaagtagcagtacctggggagctgacacttctgtaaaactagaagataaaccaggcaacgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttagctgtctccttgccaagccccaggaagaaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttga

SEQ ID NO: 2 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 3 - protein, 316 aa, Homo sapiens
MDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKVAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQD

SEQ ID NO: 4 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLLIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 5 - protein, 1298 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 6 - DNA, 31 bp, Artificial Sequence
  Primers
cagcatatggatccagatgaactcccattgg

SEQ ID NO: 7 - DNA, 34 bp, Artificial Sequence
  Primers
gcggtcgacttaaacaggaggagagctcagtgtg

SEQ ID NO: 8 - DNA, 33 bp, Artificial Sequence
  Primers
gcacatatggaacgactgccttatgatgccagc

SEQ ID NO: 9 - DNA, 38 bp, Artificial Sequence
  Primers
cctgtcgacttatccagaatcctcttccatgctcaaag

SEQ ID NO: 10 - DNA, 30 bp, Artificial Sequence
  Primers
ctcagcaggattgataagactacattgttc

SEQ ID NO: 11 - DNA, 36 bp, Artificial Sequence
  Primers
gaatttgtcccctacaaggaagctcctgaagatctg